(19) United States
(12) Patent Application Publication
(10) Pub. No.: US 2023/0382924 A1
(43) Pub. Date: Nov. 30, 2023

(54) MODULATORS OF CYSTIC FIBROSIS TRANSMEMBRANE CONDUCTANCE REGULATOR

(71) Applicant: VERTEX PHARMACEUTICALS INCORPORATED, Boston, MA (US)

(72) Inventors: Jason MCCARTNEY, Cardiff by the Sea, CA (US); Alexander Russell ABELA, Escondido, CA (US); Sunny ABRAHAM, San Diego, CA (US); Corey Don ANDERSON, Brighton, MA (US); Vijayalaksmi ARUMUGAM, San Marcos, CA (US); Jaclyn CHAU, San Diego, CA (US); Jeremy CLEMENS, San Diego, CA (US); Thomas CLEVELAND, San Marcos, CA (US); Timothy A. DWIGHT, Quincy, MA (US); Bryan A. FRIEMAN, La Jolla, CA (US); Peter GROOTENHUIS, Del Mar, CA (US); Sara Sabina HADIDA RUAH, La Jolla, CA (US); Yoshihiro ISHIHARA, San Diego, CA (US); Paul KRENITSKY, San Francisco, CA (US); Mark Thomas MILLER, Rancho Santa Fe, CA (US); Fabrice PIERRE, La Jolla, CA (US); Alina SILINA, Needham, MA (US); Johnny UY, San Diego, CA (US); Jinglan Zhou, San Diego, CA (US)

(21) Appl. No.: 18/030,527

(22) PCT Filed: Oct. 6, 2021
(86) PCT No.: PCT/US2021/053856

§ 371 Date: Apr. 6, 2023

Related U.S. Application Data
(60) Provisional application No. 63/088,874, filed on Oct. 7, 2020.

(51) Int. Cl. C07D 515/08; C07F 7/08; C07F 9/6561; C07D 515/18; C07D 515/20; C07D 519/00; A61K 31/47; A61K 31/529; A61K 31/695; A61K 31/675; A61K 31/5377; A61K 31/541

(52) U.S. Cl. CPC C07D 515/08; C07F 7/0812; C07F 9/6561; C07D 515/18; C07D 515/20; C07D 519/00; A61K 31/47; A61K 31/529; A61K 31/695; A61K 31/675; A61K 31/5377; A61K 31/541; C07B 2200/09

(57) ABSTRACT

This disclosure provides modulators of Cystic Fibrosis Transmembrane Conductance Regulator (CFTR)having the core structure: pharmaceutical compositions containing at least one such modulator, methods of treating CFTR mediated diseases, including cystic fibrosis, using such modulators and pharmaceutical compositions, combination therapies, and processes and intermediates for making such modulators.

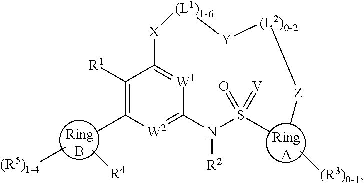

This application claims the benefit of priority of U.S. Provisional Application No. 63/088,874, filed Oct. 7, 2020, the content of which is incorporated by reference herein in its entirety.

The disclosure relates to modulators of Cystic Fibrosis Transmembrane Conductance Regulator (CFTR), pharmaceutical compositions containing the modulators, methods of treating CFTR mediated diseases, including cystic fibrosis, using such modulators and pharmaceutical compositions, combination therapies and combination pharmaceutical compositions employing such modulators, and processes and intermediates for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺-K⁺-ATPase pump and Cl-channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na^{+/}2Cl⁻/K⁺ co-transporter, Na⁺-K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of the cystic fibrosis and other CFTR mediated diseases, and particularly the more severe forms of these diseases, are still needed.

One aspect of the disclosure provides novel compounds, including compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing.

Formula I encompasses compounds falling within the following structure:

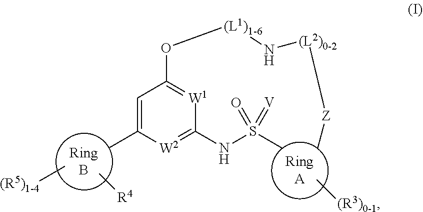

and includes tautomers of those compounds, deuterated derivatives of any of the compounds and tautomers, and pharmaceutically acceptable salts of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl;
Ring B is selected from:
   C₆-C₁₀ aryl,
   C₃-C₁₀ cycloalkyl,
   3- to 10-membered heterocyclyl, and
   5- to 10-membered heteroaryl;
V is selected from O and NH;
W¹ is selected from N and CH;
W² is selected from N and CH; provided that at least one of W¹ and W² is N;
Z is selected from O, NR^{ZN}, and C(R^{ZC})₂, provided that when L² is absent, Z is C(R^{ZC})₂;
each L¹ is independently selected from C(R^{L1})₂ and

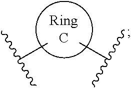

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(R^{N})₂,
   —SO—Me,
   —CH═C(R^{Lc})₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy, and
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         (O)₀₋₁(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl (optionally substituted with 1-3 groups independently selected from oxo and C₁-C₆ alkoxy),
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3-SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-me member heterocyclyl optionally substituted with 1-3 groups independently selected from C₁-C₆ alkyl, and
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl, and
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F};
each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F}.
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      NHCOMe,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl, and
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂, and
      NHMe, and
      C₁-C₆ alkyl optionally substituted with 1-3 groups independently selected from hydroxyl, and
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
   hydroxyl,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
         oxo,
         halogen,
         hydroxyl,
         N(R^{N})₂,
         —SO₂—(C₁-C₆ alkyl),
         C₁-C₆ alkoxy optionally substituted with 1-3 groups independently selected from halogen, C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
   5- to 12-membered heteroaryl optionally substituted with 1-3 groups independently selected from C₁-C₆ alkyl and C₁-C₆ fluoroalkyl.

Formula I also includes compounds of Formula Ia:

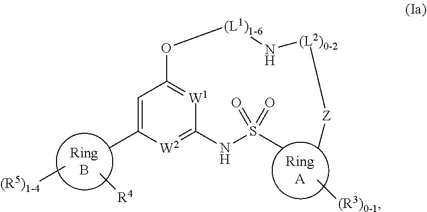

tautomers of those compounds, deuterated derivatives of any of the compounds and tautomers, and pharmaceutically acceptable salts of any of the foregoing, wherein Ring A, Ring B, W¹, W², Z, L¹, L², R³, R⁴, and R⁵ are as defined for Formula I.

Formula I also includes compounds of Formula IIa:

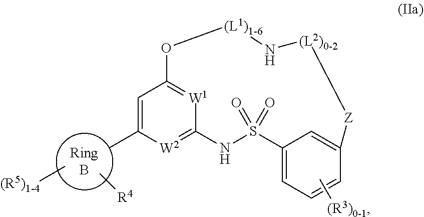

tautomers of those compounds, deuterated derivatives of any of the compounds and tautomers, and pharmaceutically acceptable salts of any of the foregoing, wherein Ring B, W¹, W², Z, L¹, L², R³, R⁴, and R⁵ are as defined for Formula I.

Formula I also includes compounds of Formula IIb:

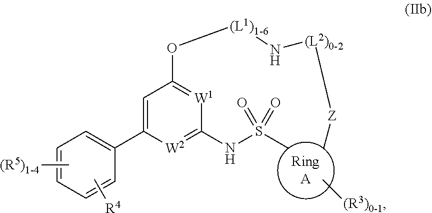

tautomers of those compounds, deuterated derivatives of any of the compounds and tautomers, and pharmaceutically acceptable salts of any of the foregoing, wherein Ring A, W¹, W², Z, L¹, L², R³, R⁴, and R⁵ are as defined for Formula I.

Formula I also includes compounds of Formula III:

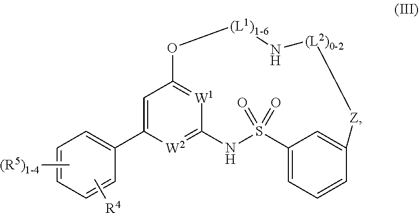

tautomers of those compounds, deuterated derivatives of any of the compounds and tautomers, and pharmaceutically acceptable salts of any of the foregoing, wherein W¹, W², Z, L¹, L², R⁴, and R⁵ are as defined for Formula I.

Formula I also encompasses compounds of Formula IV:

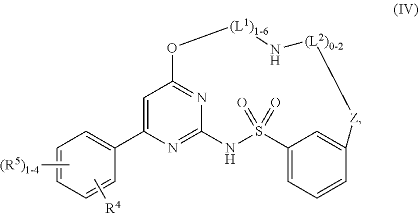

tautomers of those compounds, deuterated derivatives of any of the compounds and tautomers, and pharmaceutically acceptable salts of any of the foregoing, wherein W¹, W², Z, L¹, L², R⁴, and R⁵ are as defined for Formula I.

Formula I also includes compounds of Formula V:

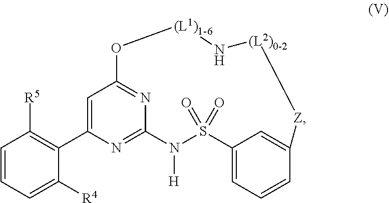

tautomers of those compounds, deuterated derivatives of any of the compounds and tautomers, and pharmaceutically acceptable salts of any of the foregoing, wherein W¹, W², Z, L¹, L², R³, R⁴, and R⁵ are as defined for Formula I.

Formula I also encompasses compounds of Formula VI:

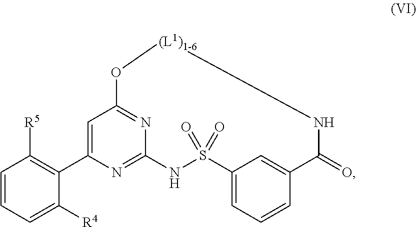

tautomers of those compounds, deuterated derivatives of any of the compounds and tautomers, and pharmaceutically acceptable salts of any of the foregoing, wherein L¹, R⁴, and R⁵ are as defined for Formula I.

Another aspect of the disclosure provides pharmaceutical compositions comprising at least one compound chosen from the novel compounds disclosed herein, tautomers thereof, deuterated derivatives of those compounds and tautomers, and pharmaceutically acceptable salts of the foregoing, and at least one pharmaceutically acceptable carrier, which compositions may further include at least one additional active pharmaceutical ingredient. In some embodiments, the at least one additional active pharmaceutical ingredient is at least one other CFTR modulator. In some embodiments, the at least one other CFTR modulator is selected from CFTR potentiators. In some embodiments, the at least one other CFTR modulator is selected from CFTR correctors. In some embodiments, the at least one other CFTR modulators includes both a potentiator and a corrector. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

Thus, another aspect of the disclosure provides methods of treating the CFTR-mediated disease cystic fibrosis comprising administering at least one of compound chosen from the novel compounds disclosed herein, tautomers thereof, deuterated derivatives of those compounds and tautomers, and pharmaceutically acceptable salts of the foregoing, and at least one pharmaceutically acceptable carrier, optionally as part of a pharmaceutical composition comprising at least one additional active pharmaceutical ingredient, to a subject in need thereof. In some embodiments, the at least one additional active pharmaceutical ingredient is at least one other CFTR modulator. In some embodiments, the at least one other CFTR modulator is selected from CFTR potentiators. In some embodiments, the at least one other CFTR modulator is selected from CFTR correctors. In some embodiments, the at least one other CFTR modulators includes both a potentiator and a corrector. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

In certain embodiments, the pharmaceutical compositions of the disclosure comprise at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing. In some embodiments, compositions comprising at least one compound chosen from compounds of Formulae I, Ia, IIa, IIb, III, IV, V, and VI, tautomers thereof, deuterated derivatives of those compounds and tautomers, and pharmaceutically acceptable salts of any of the foregoing may optionally further comprise (a) at least one compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor) 3-(6-(1-(2,2-difluorobenzo[d][1,3]dioxol-5-yl)cyclopropane carboxamido)-3-methylpyridin-2-yl)benzoic acid (lumacaftor) and deuterated derivatives and pharmaceutically acceptable salts of tezacaftor and lumacaftor; and/or(b) at least one compound chosen from N-[2,4-bis(1,1-dimethylethyl)-5-hydroxyphenyl]-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

Another aspect of the disclosure provides methods of treating the CFTR-mediated disease, cystic fibrosis, that comprise administering to a patient in need thereof at least one compound chosen from the novel compounds disclosed herein, deuterated derivatives thereof and pharmaceutically acceptable salts of any of the foregoing, and optionally further administering one or more additional CFTR modulating agents. A further aspect of the disclosure provides the pharmaceutical compositions of the disclosure comprising at least one compound chosen from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing and, optionally, one or more CFTR modulating agents, for use in therapy or for use in the manufacture of a medicament. In some embodiments the optional one or more additional CFTR modulating agents are selected from CFTR potentiators. In some embodiments, the one or more additional CFTR modulating agents are selected from CFTR correctors. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

A further aspect of the disclosure provides intermediates and methods for making the compounds and pharmaceutical compositions disclosed herein.

### Definitions

“Tezacaftor” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

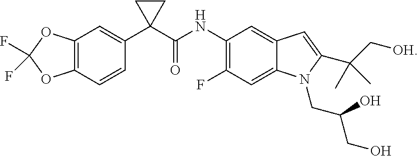 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative or a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each incorporated hereing by reference.

“Ivacaftor” as used throughout this disclosure refers to N-(2,4-di-tert-butyl-5-hydroxyphenyl)-1,4-dihydro-4-oxoquinoline-3-carboxamide, which is depicted by the structure:

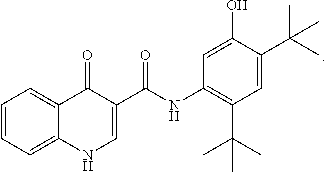 [SMILES: CC(C)(C)c1cc(C(C)(C)C)c(NC(=O)c2c[nH]c3ccccc3c2=O)cc1O]

Ivacaftor may also be in the form of a deuterated derivative, pharmaceutically acceptable salt, of pharmaceutically acceptable salt of a deuterated derivative. Ivacaftor and methods of making and using ivacaftor are disclosed in WO 2006/002421, WO 2007/079139, WO 2010/108162, and WO 2010/019239, each incorporated herein by reference.

In some embodiments, a specific deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

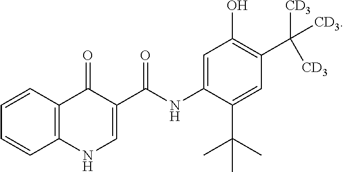 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of a further deuterated derivative, a pharmaceutically acceptable salt, or a pharmaceutically acceptable salt of a deuterated derivative. Deutivacaftor and methods of making and using deutivacaftor are disclosed in WO 2012/158885, WO 2014/078842, and U.S. Pat. No. 8,865,902, incorporated herein by reference.

“Lumacaftor” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

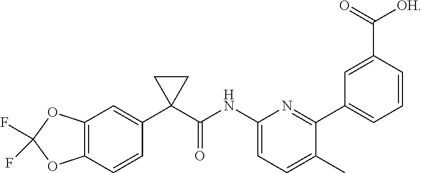 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using Lumacaftor are disclosed in WO 2007/056341, WO 2009/073757, and WO 2009/076142, each of which is incorporated herein by reference.

As used herein, the term “alkyl” refers to a saturated or partially saturated, branched or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms) in which the bond between one or more adjacent carbon atoms is a double (alkenyl) or triple (alkynl) bond. Alkyl groups may be substituted or unsubstituted.

As used herein, the term “haloalkyl group” refers to an alkyl group substituted with one or more halogen atoms, e.g., fluoroalkyl, which refers to an alkyl group substituted with one or more fluorine atoms.

The term “alkoxy,” as used herein, refers to an alkyl or cycloalkyl covalently bonded to an oxygen atom. Alkoxy groups may be substituted or unsubstituted.

As used herein, the term “haloalkoxyl group” refers to an alkoxy group substituted with one or more halogen atoms.

As used herein, “cycloalkyl” refers to a cyclic, bicyclic, tricyclic, or polycyclic non-aromatic hydrocarbon groups having 3 to 12 carbons (such as, for example 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, dispiro[2.0.2.1]heptane, and spiro[2,3]hexane. Cycloalkyl groups may be substituted or unsubstituted.

The term “aryl,” as used herein, is a functional group or substituent derived from an aromatic ring and encompasses monocyclic aromatic rings and bicyclic, tricyclic, and fused ring systems wherein at least one ring in the system is aromatic. Non-limiting examples of aryl groups include phenyl, naphthyl, and 1,2,3,4-tetrahydronaphthalenyl.

The term “heteroaryl ring,” as used herein, refers to an aromatic ring comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) wherein at least one ring in the system is aromatic. Non-limiting examples of heteroaryl rings include pyridine, quinoline, indole, and indoline.

As used herein, the term “heterocyclyl ring” refers to a non-aromatic hydrocarbon containing 3 to 12 atoms in a ring (such as, for example 3-10 atoms) comprising at least one ring atom that is a heteroatom, such as O, N, or S and may include one or more unsaturated bonds. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Non-limiting examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group.

As used herein, “CFTR” means cystic fibrosis transmembrane conductance regulator.

The terms “CFTR modulator” and “CFTR modulating agent” are used interchangeably herein to refer to a compound that increases the activity of CFTR. The increase in activity resulting from a CFTR modulator includes but is not limited to compounds that correct, potentiate, stabilize and/or amplify CFTR.

The terms “corrector” and “CFTR corrector” are used interchangeably to refer to a compound that facilitates the processing and trafficking of CFTR to increase the amount of CFTR at the cell surface. The novel compounds disclosed herein are CFTR correctors. Other correctors may be used in combination therapies with the novel compounds disclosed herein to treat CFTR mediated diseases, such as cystic fibrosis. Such other correctors include, e.g., tezacaftor, lumacaftor, and their deuterated derivatives and pharmaceutically acceptable salts.

The term “potentiator” and “CFTR potentiator” are used interchangeably to refer to a compound that increases the channel activity of CFTR protein located at the cell surface, resulting in enhanced ion transport. Ivacaftor and deutivacaftor disclosed herein are CFTR potentiators. Potentiators may be used in combination with the novel compounds of the disclosure to treat CFTR mediated diseases such as cystic fibrosis. Such potentiators include, e.g., ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and their deuterated derivatives and pharmaceutically acceptable salts.

It will be appreciated that when a description of a combination of compound selected from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing, and other specified CFTR modulating agents is provided herein, typically, but not necessarily, the combination or treatment regime will include at least one potentiator, such as, e.g., a potentiator selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof. It will also be appreciated that typically, but not necessarily, a single potentiator may used in a combination pharmaceutical composition or therapy. In some embodiments, a combination of at least one compound selected from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing, and other specified CFTR modulating agents, will also include another CFTR corrector, such as, e.g., a corrector compound selected from tezacaftor, lumacaftor, and deuterated derivatives and pharmaceutically acceptable salts thereof.

The term “at least one compound selected from,” as used herein, refers to the selection of one or more of the compounds from a specified group.

A reference to “Compounds 1-496 in this disclosure is intended to represent a reference to each of Compounds 1 through 496 individually.

As used herein, the term “active pharmaceutical ingredient” or “therapeutic agent” (“API”) refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

It should be understood that references herein to methods of treatment (e.g., methods of treating a CFTR mediated disease or a method of treating cystic fibrosis) using one or more compounds of the disclosure optionally in combination with one or more additional CFTR modulating agents (e.g., a compound chosen from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing, optionally in combination with one or more additional CFTR modulating agents) should also be interpreted as references to:
one or more compounds (e.g., compound chosen from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing, optionally in combination with one or more additional CFTR modulating agents) for use in methods of treating, e.g., cystic fibrosis optionally in combination with one or more additional CFTR modulating agents; and/or
the use of one or more compounds (e.g., a compound chosen from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing, optionally in combination with one or more additional CFTR modulating agents) in the manufacture of a medicament for treating, e.g., cystic fibrosis.

It should be also understood that references herein to methods of treatment (e.g., methods of treating a CFTR mediated disease or a method of treating cystic fibrosis) using a pharmaceutical composition of the disclosure (e.g., a pharmaceutical composition comprising at least one compound chosen from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing and optionally further comprising one or more additional CFTR modulating agents) should also be interpreted as references to:
a pharmaceutical composition (e.g., a pharmaceutical composition comprising at least one compound chosen from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing and optionally further comprising one or more additional CFTR modulating agents) for use in methods of treating, e.g., cystic fibrosis; and/or
the use of a pharmaceutical composition (e.g., a pharmaceutical composition comprising at least one compound chosen from compounds of Formula I, compounds of any one of Formulae Ia, IIa, IIb, III, IV, V, and VI, Compounds 1-496, tautomers thereof, deuterated derivatives of those compounds and tautomers, and pharmaceutically acceptable salts of any of the foregoing and optionally further comprising one or more additional CFTR modulating agents) in the manufacture of a medicament for treating, e.g., cystic fibrosis.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately” may refer to an acceptable error for a particular value as determined by one of skill in the art, which depends in part on how the values is measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product >1 g/l).

As used herein, the term “room temperature” or “ambient temperature” means 15° C. to 30° C.

It will be appreciated that certain compounds of this disclosure may exist as separate stereoisomers or enantiomers and/or mixtures of those stereoisomers or enantiomers.

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof:

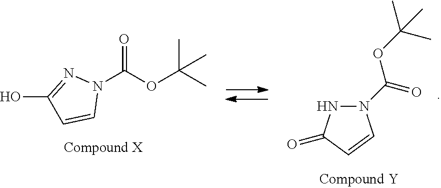

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “and deuterated derivatives and pharmaceutically acceptable salts thereof” is used interchangeably with “and deuterated derivatives and pharmaceutically acceptable salts thereof of any of the forgoing” in reference to one or more compounds or formulae of the disclosure. These phrases are intended to encompass pharmaceutically acceptable salts of any one of the referenced compounds, deuterated derivatives of any one of the referenced compounds, and pharmaceutically acceptable salts of those deuterated derivatives.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

### Methods of Treatment

Any of the novel compounds disclosed herein, such as for example, compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the disclosure provides methods of treating, lessening the severity of, or symptomatically treating cystic fibrosis in a patient comprising administering to the patient an effective amount of any of the novel compounds disclosed herein, such as for example, compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, alone or in combination with another active ingredient, such as one or more CFTR modulating agents. In some embodiments, the one (or more) CFTR modulating agent is a corrector. In some embodiments, the one (or more) CFTR modulating agent is a potentiator. In some embodiments, the CFTR modulating agents include both a corrector and a potentiator. In some embodiments, the one or more CFTR modulating agents are selected from potentiators: ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing; and correctors: lumacaftor, tezacaftor, and deuterated derivatives and pharmaceutically acceptable salts thereof.

In some embodiments, 5 mg to 500 mg of a compound disclosed herein, a tautomer thereof, a deuterated derivative of the compound and tautomer, or a pharmaceutically acceptable salt of any of the foregoing are administered daily.

In some embodiments, the patient to be treated has an F508del/minimal function (MF) genotype, F508del/F508del genotype (homozygous for the F508del mutation), F508del/gating genotype, or F508del/residual function (RF) genotype. In some embodiments the patient is heterozygous and has one F508del mutation. In some embodiments the patient is homozygous for the N1303K mutation.

In some embodiments, the patient to be treated has at least one F508del mutation in the CFTR gene. In some embodiments, the patient has a CFTR gene mutation that is responsive to a compound, tautomer, deutrated derivative, or pharmaceutically acceptable salt of the disclosurebased on in vitro data. In some embodiments, the patient is heterozygous and has an F508del mutation on one allele and a mutation on the other allele selected from Table 2:

TABLE 2
CFTR Mutations

| MF Category | Mutation | | | | |
|---|---|---|---|---|---|
| Nonsense | Q2X | L218X | Q525X | R792X | E1104X |
| mutations | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical | 185 + 1G→T | 711 + 5G→A | 1717 − 8G→A | 2622 + 1G→A | 3121 − 1G→A |
| splice | 296 + 1G→A | 712 − 1G→T | 1717 − 1G→A | 2790 − 1G→C | 3500 − 2A→G |
| mutations | 296 + 1G→T | 1248 + 1G→A | 1811 + 1G→C | 3040G→C | 3600 + 2insT |
| | 405 + 1G→A | 1249 − 1G→A | 1811 + 1.6 kbA→G | (G970R) | 3850 − 1G→A |
| | 405 + 3A→C | 1341 + 1G→A | 1811 + 1643G→T | 3120G→A | 4005 + 1G→A |
| | 406 − 1G→A | 1525 − 2A→G | 1812 − 1G→A | 3120 + 1G→A | 4374 + 1G→T |
| | 621 + 1G→T | 1525 − 1G→A | 1898 + 1G→A | 3121 − 2A→G | |
| | 711 + 1G→T | | 1898 + 1G→C | | |
| Small (≤3 | 182delT | 1078delT | 1677delTA | 2711delT | 3737delA |
| nucleotide) | 306insA | 1119delA | 1782delA | 2732insA | 3791delC |
| insertion/deletion | 306delTAGA | 1138insG | 1824delA | 2869insG | 3821delT |
| (ins/del) | 365-366insT | 1154insTC | 1833delT | 2896insAG | 3876delA |
| frameshift | 394delTT | 1161delC | 2043delG | 2942insT | 3878delG |
| mutations | 442delA | 1213delT | 2143delT | 2957delT | 3905insT |
| | 444delA | 1259insA | 2183AA→G ^{a} | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |
| Non-small (>3 | CFTRdele1 | | CFTRdele16-17b | 1461ins4 | |
| nucleotide) | CFTRdele2 | | CFTRdele17a, 17b | 1924del7 | |
| insertion/deletion | CFTRdele2, 3 | | CFTRdele17a-18 | 2055del9→A | |
| (ins/del) | CFTRdele2-4 | | CFTRdele19 | 2105-2117del13insAGAAA | |
| frameshift | CFTRdele3-10, 14b-16 | | CFTRdele19-21 | 2372del8 | |
| mutations | CFTRdele4-7 | | CFTRdele21 | 2721del11 | |
| | CFTRdele4-11 | | CFTRdele22-24 | 2991del32 | |
| | CFTR50 kbdel | | CFTRdele22, 23 | 3667ins4 | |
| | CFTRdup6b-10 | | 124del23bp | 4010del4 | |
| | CFTRdele11 | | 602del14 | 4209TGTT→AA | |
| | CFTRdele13, 14a | | 852del22 | | |
| | CFTRdele14b-17b | | 991del5 | | |
| Missense | A46D | V520F | Y569D | N1303K | |
| mutations that | G85E | A559T | L1065P | | |
| Are not responsive | R347P | R560T | R1066C | | |
| in vitro to TEZ, | L467P | R560S | L1077P | | |
| IVA, or TEZ/IVA | I507del | A561E | M1101K | | |
| and | | | | | |
| % PI >50% and | | | | | |
| SwCl⁻ >86 | | | | | |
| mmol/L | | | | | |

^{a} Also known as 2183delAA→G.
CFTR: cystic fibrosis transmembrane conductance regulator;
IVA: ivacaftor.
SwCl: sweat chloride.
TEZ: tezacaftor.
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 May 2018.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (2H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as for example, compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein includes Pulmozyme®.

In some embodiments, the additional agent is a bronchodilator. Exemplary bronchodiltors include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreatic enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

In some embodiments, at least one additional active pharmaceutical ingredient is selected from CFTR modulating agents. In some embodiments, the additional active pharmaceutical ingredient is selected from CFTR potentiators. In some embodiments, the potentiator is selected from ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the additional active pharmaceutical ingredient is chosen from CFTR correctors. In some embodiments, the correctors are selected from lumacaftor, tezacaftor, deuterated derivatives of lumacaftor and tezacaftor, and pharmaceutically acceptable salts of any of the foregoing. In some embodiments, the additional active pharmaceutical ingredient includes both a CFTR potentiator and a CFTR corrector.

In some embodiments, the at least one additional active pharmaceutical ingredient is chosen from (a) tezacaftor, lumacaftor, and deuterated derivatives and pharmaceutically acceptable salts thereof; and/or (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and pharmaceutically acceptable salts of any of the foregoing. Thus, in some embodiments, the combination therapies provided herein comprise (a) a compound selected from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; or (c) at least one compound selected from ivacaftor, deutivacaftor, and deuterated derivatives and pharmaceutically acceptable salts thereof. In some embodiments, the combination therapies provided herein comprise (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; and (c) at least one compound selected from ivacaftor, deutivacaftor, and deuterated derivatives and pharmaceutically acceptable salts thereof. In some embodiments, the combination therapies provided herein comprise (a) at least one compound chosen from compounds of Formula I, compounds of Formulae Ia, IIa, IIb, III, IV, V, and VI, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof, and (c) at least one compound selected from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof. some embodiments, at least one compound chosen from compounds of compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from ivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from deutivacaftor and deuterated derivatives and pharmaceutically acceptable salts thereof. In some embodiments, at least one compound chosen from compounds of compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosed from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosed from lumacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

Each of the compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, independently can be administered once daily, twice daily, or three times daily. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered once daily. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from lumcafter and deuterated derivatives and pharmaceutically acceptable salts thereof are administered once daily. In some embodiments, at least one compound chosen from compounds of compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from ivacaftor, deutivacaftor, and deuterated derivatives and pharmaceutically acceptable salts thereof are administered once daily. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from ivacaftor, deutivacaftor, and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered once daily. In some embodiments, at least one compound chosen from compounds of compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor deutivacaftor, and deuterated derivatives and pharmaceutically acceptable salts thereof are administered once daily. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, at least one compound chosen from ivacaftor, deutivacaftor, and deuterated derivatives and pharmaceutically acceptable salts thereof, and at least one compound chosen from lumacaftor and pharmaceutically acceptable salts thereof, are administered once daily. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, at least one compound chosen from ivacaftor, deutivacaftor, and deuterated derivatives and pharmaceutically acceptable salts thereof, and at least one compound chosen from lumacaftor and deuterated derivatives and pharmaceutically acceptable salts thereof, are administered twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, at least one compound chosen from tezacaftor, lumacaftor, and deuterated derivatives and pharmaceutically acceptable salts thereof, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, are administered once or twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from ivacaftor and deuterated derivatives and pharmaceutically acceptable salts thereof, are administered twice daily. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof, are administered once daily and at least one compound chosen from ivacaftor and pharmaceutically acceptable salts thereof, are administered twice daily.

Compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof can be administered in a single pharmaceutical composition or separate pharmaceutical compositions. Such pharmaceutical compositions can be administered once daily or multiple times daily, such as twice daily. As used herein, the phrase that a given amount of API (e.g., tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or a deuterated derivative or pharmaceutically acceptable salt thereof) “is administered once or twice daily or per day” means that said given amount is administered per dosing once or twice daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; and at least one compound chosen from ivacaftor and deuterated derivatives and pharmaceutically acceptable salts thereof is administered in a third pharmaceutical composition.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from deutivacaftor and deuterated derivatives and pharmaceutically acceptable salts thereof is administered in a third pharmaceutical composition.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof is administered in a third pharmaceutical composition.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in a first pharmaceutical composition; at least one compound chosen from ivacftor, deutivacaftor, and deuterated derivatives and pharmaceutically acceptable salts thereof is administered in a second pharmaceutical composition; at least one compound chosen from lumacaftor and deuterated derivatives and pharmaceutically acceptable salts thereof is administered in a third pharmaceutical composition. In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in a first pharmaceutical composition; at least one compound chosen from lumacaftor, and deuterated derivatives and pharmaceutically acceptable salts thereof is administered in a second pharmaceutical composition; at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof is administered in a third pharmaceutical composition.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, is administered in a first pharmaceutical composition; and at least one compound chosen from tezacaftor and deuterated derivatives and pharmaceutically acceptable salts thereof and at least one compound chosen from ivacaftor, deutivacaftor, and deuterated derivatives and pharmaceutically acceptable salts thereof are administered in a second pharmaceutical composition. In some embodiments, the second pharmaceutical composition comprises a half of a daily dose of ivacaftor, and the other half dose of ivacaftor hereof is administered in a third pharmaceutical composition.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing; at least one compound chosen from tezacaftor and deuterated derivatives and pharmaceutically acceptable salts thereof and at least one compound chosen from ivacaftor, deutivacaftor, and deuterated derivatives and pharmaceutically acceptable salts thereof are administered in a first pharmaceutical composition. In some embodiments, the first pharmaceutical composition is administered to the patient twice daily. In some embodiments the first pharmaceutical composition is administered once daily. In some embodiments the first pharmaceutical composition is administered once daily and a second composition comprising only ivacaftor is administered once daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing; at least one compound chosen from lumacaftor and deuterated derivatives and pharmaceutically acceptable salts thereof and at least one compound chosen from ivacaftor, deutivacaftor, and deuterated derivatives and pharmaceutically acceptable salts thereof are administered in a first pharmaceutical composition. In some embodiments, the first pharmaceutical composition is administered to the patient twice daily. In some embodiments the first pharmaceutical composition is administered once daily. In some embodiments the first pharmaceutical composition is administered once daily and a second composition comprising only ivacaftor is administered once daily.

In some embodiments, at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing; at least one compound chosen from tezacaftor, lumacaftor, and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered in a first pharmaceutical composition. In some embodiments, the first pharmaceutical composition is administered to the patient twice daily. In some embodiments the first pharmaceutical composition is administered once daily.

Any suitable pharmaceutical compositions can be used for compounds of Formula I, compounds of Formulae Ia, IIa, IIb, III, IV, V, and VI, Compounds 1-496, tezacaftor, ivacaftor, deutivacaftor, lumacaftor and tautomers thereof, deuterated derivatives of those compounds and tautomers, and pharmaceutically acceptable salts of any of the foregoing. Some exemplary pharmaceutical compositions for tezacaftor and its deuterated derivatives and pharmaceutically acceptable salts can be found in WO 2011/119984 and WO 2014/014841, wach of which is incorporated herein by reference. Some exemplary pharmaceutical compositions for ivacaftor and its deuterated derivatives and pharmaceutically acceptable salts can be found in WO 2007/134279, WO 2010/019239, WO 2011/019413, WO 2012/027731, and WO 2013/130669, and some exemplary pharmaceutical compositions for deutivacaftor and its deuterated derivatives and pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its deuterated derivatives and pharmaceutically acceptable salts can be found in WO 2010/037066, WO 2011/127421, and WO 2014/071122, incorporated herein by reference.

### Pharmaceutical Compositions

Another aspect of the disclosure provides a pharmaceutical composition comprising at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least one pharmaceutically acceptable carrier.

In some embodiments, the disclosure provides pharmaceutical compositions comprising at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the disclosure provides a pharmaceutical composition comprising (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, (b) at least one compound chosen from ivacaftor, deutivacaftor, and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, (b) at least one compound chosen from lumacaftor and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, (b) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from ivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from deutivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, (b) at least one compound chosen from ivacaftor, deutivacaftor, and deuterated derivatives and pharmaceutically acceptable salts thereof, (c) at least one compound chosen from lumacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-496, tautomers thereof, deuterated derivatives of those compounds and tautomers, and pharmaceutically acceptable salts of any of the foregoing, (b) at least one compound chosen from tezacaftor, lumacaftor, and deuterated derivatives and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Exemplary Embodiments

A non-limiting list of embodiments is provided below:
1. A compound of Formula I:

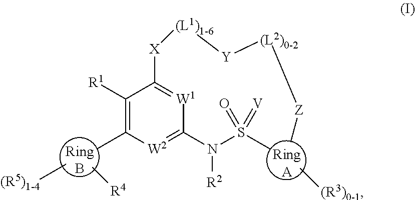

a tautomer thereof, a deuterated derivative of the compound or tautomer, or a pharmaceutically acceptable salt of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl; Ring B is selected from:
C₆-C₁₀ aryl,
   C₃-C₁₀ cycloalkyl,
   3- to 10-membered heterocyclyl, and
   5- to 10-membered heteroaryl;
V is selected from O and NH;
W¹ is selected from N and CH;
W² is selected from N and CH; provided that at least one of W¹ and W² is N;
Z is selected from O, NR^{ZN}, and C(R^{ZC})₂, provided that when L² is absent, Z is C(R^{ZC})₂;
each L¹ is independently selected from C(R^{L1})₂ and

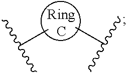

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(R^{N})₂,
   —SO—Me,
   —CH═C(R^{L}c)₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy, and
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl (optionally substituted with 1-3 groups independently selected from oxo and C₁-C₆ alkoxy),
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3-SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-member heterocyclyl optionally substituted with 1-3 groups independently selected from C₁-C₆ alkyl, and
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl, and
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F};
each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl), and
   C₆-C₁₀ aryl optionally substituted with 1-3 groups independently selected from C₁-C₆ alkyl, and
   R^{F};
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F}.
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      NHCOMe,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl, and
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂, and
      NHMe, and
      C₁-C₆ alkyl optionally substituted with 1-3 groups independently selected from hydroxyl,
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
   hydroxyl,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
         oxo,
         halogen,
         hydroxyl,
         N(R^{N})₂,
         SO₂—(C₁-C₆ alkyl),
         C₁-C₆ alkoxy optionally substituted with 1-3 groups independently selected from halogen, C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         —O—(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
   5- to 12-membered heteroaryl optionally substituted with 1-3 groups independently selected from C₁-C₆ alkyl and C₁-C₆ fluoroalkyl.
2. The compound, tautomer, deuterated derivative, or pharmaceutically acceptable salt according to embodiment 1, wherein Ring A is selected from selected from C₆-C₁₀ aryl, 3- to 10-membered heterocyclyl, and 5- to 10-membered heteroaryl.
3. The compound, tautomer, deuterated derivative, or pharmaceutically acceptable salt according to embodiment 1 or 2, wherein Ring A is selected from phenyl, pyrazolyl, pyridyl, piperidinyl, and isoxazolyl.
4. The compound, tautomer, deuterated derivative, or pharmaceutically acceptable salt according to any one of embodiments 1 to 3, wherein Ring A is phenyl.
5. The compound, tautomer, deuterated derivative, or pharmaceutically acceptable salt according to any one of embodiments 1 to 4, wherein Ring B is selected from C₆-C₁₀ aryl, C₃-C₁₀ cycloalkyl, and 5- to 10-membered heteroaryl.
6. The compound, tautomer, deuterated derivative, or pharmaceutically acceptable salt according to any one of embodiments 1 to 5, wherein Ring B is selected from phenyl, napthyl, pyridinyl, pyrimidinyl, pyrazoyl, cyclopropyl, cyclobutyl, cyclohexyl, cyclohexenyl, pyrrolidinyl, 3,4-dihydro-2H-pyranyl, 3,6-dihydro-2H-pyranyl, cyclopentenyl, norbornenyl, and tetrahydropyranyl.
7. The compound, tautomer, deuterated derivative, or pharmaceutically acceptable salt according to any one of embodiments 1 to 6, wherein Ring B is phenyl.
8. The compound, tautomer, deuterated derivative, or pharmaceutically acceptable salt according to any one of embodiments 1 to 7, wherein V is O.
9. The compound, tautomer, deuterated derivative, or pharmaceutically acceptable salt according to any one of embodiments 1 to 8, wherein W¹ is N and W² is N.
10. The compound, tautomer, deuterated derivative, or pharmaceutically acceptable salt according to any one of embodiments 1 to 8, wherein W¹ is CH and W² is N.
11. The compound, tautomer, deuterated derivative, or pharmaceutically acceptable salt according to any one of embodiments 1 to 10, wherein Z is selected from NR^{ZN} and C(R^{ZC})₂, provided that when L² is absent, Z is C(R^{ZC})₂
12. The compound, tautomer, deuterated derivative, or pharmaceutically acceptable salt according to any one of embodiments 1 to 11, wherein Ring C is a phenyl optionally substituted with 1-3 groups independently selected from:
   halogen,
   C₁-C₆ alkyl, and
   N(R^{N})₂.
13. The compound, tautomer, deuterated derivative, or pharmaceutically acceptable salt according to any one of embodiments 1 to 12, wherein each R³ is independently selected from:
   C₁-C₆ alkyl,
   C₃-C₁₀ cycloalkyl,
   C₆-C₁₀ aryl optionally substituted with 1-3 groups independently selected from C₁-C₆ alkyl, and
   3- to 10-membered heterocyclyl.
14. The compound, tautomer, deuterated derivative, or pharmaceutically acceptable salt according to any one of embodiments 1 to 13, wherein each R³ is independently selected from methyl, cyclopropyl, cyclohezyl, cyclohexenyl, tetrahyropyranyl, and 4-(tert-butyl)phenyl.
15. The compound, tautomer, deuterated derivative, or pharmaceutically acceptable salt according to any one of embodiments 1 to 12, wherein R³ is absent.
16. The compound, tautomer, deuterated derivative, or pharmaceutically acceptable salt according to any one of embodiments 1 to 15, wherein R⁴ is selected from hydrogen and methyl.
17. The compound, tautomer, deuterated derivative, or pharmaceutically acceptable salt according to any one of embodiments 1 to 16, wherein R⁴ is methyl.
18. The compound, tautomer, deuterated derivative, or pharmaceutically acceptable salt according to any one of embodiments 1 to 17, wherein each R⁵ is independently selected from:
   hydrogen,
   halogen,
   C₁-C₆ alkyl optionally substituted with 1-3 groups independently selected from:
      hydroxyl,
      C₁-C₆ alkoxy optionally substituted with 1-3 groups independently selected from C₆-C₁₀ aryl,
      C₃-C₁₀ cycloalkyl, and
      —(O)₀₋₁—(C₆-C₁₀ aryl) optionally substituted with 1-3 groups independently selected from C₁-C₆ alkyl and C₁-C₆ alkoxy,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl, and
   C₆-C₁₀ aryl.
19. The compound, tautomer, deuterated derivative, or pharmaceutically acceptable salt according to any one of embodiments 1 to 18, wherein R^{ZN} is hydrogen.
20. The compound, tautomer, deuterated derivative, or pharmaceutically acceptable salt according to any one of embodiments 1 to 19, wherein R^{ZC} is hydrogen, or two R^{ZC} are taken together to form an oxo group.
21. The compound, tautomer, deuterated derivative, or pharmaceutically acceptable salt according to any one of embodiments 1 to 20, wherein each R^{L1} is independently selected from:
   hydrogen,
   C₁-C₉ alkyl optionally substituted with 1-3 groups independently selected from:
      halogen,
      hydroxyl,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl,
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from hydroxyl, cyano, and SiMe₃,
      C₁-C₆ alkoxy optionally substituted with 1-3 groups independently selected from C₃-C₁₀ cycloalkyl (optionally substituted with 1-3 groups independently selected from C₁-C₆ fluoroalkyl), and C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl, and
      3- to 10-membered heterocyclyl optionally substituted with 1-3 groups independently selected from C₁-C₆ alkyl,
   3- to 10-membered heterocyclyl optionally substituted with 1-3 groups independently selected from C₁-C₆ alkyl (optionally substituted with 1-3 groups independently selected from oxo and C₁-C₆ alkoxy), and
   5- to 10-membered heteroaryl optionally substituted with 1-3 groups independently selected from C₁-C₆ alkyl.
22. The compound, tautomer, deuterated derivative, or pharmaceutically acceptable salt according to any one of embodiments 1 to 21, wherein each R^{L2} is independently selected from hydrogen and R^{F}, or two R^{L2} on the same carbon atom are taken together to form an oxo group.
23. The compound, tautomer, deuterated derivative, or pharmaceutically acceptable salt according to any one of embodiments 1 to 22, wherein each R^{N} is independently selected from:
   hydrogen,
   C₁-C₈ alkyl optionally substituted with 1-3 groups independently selected from oxo, C₁-C₆ alkoxy, and C₆-C₁₀ aryl, and
   C₃-C₁₀ cycloalkyl,
24. The compound, tautomer, deuterated derivative, or pharmaceutically acceptable salt according to any one of embodiments 1 to 23, wherein when R^{F} is present, two R^{F} taken together with the atoms to which they are bonded form a group selected from:
   C₃-C₁₀ cycloalkyl,
   C₆-C₁₀ aryl,
   3- to 11-membered heterocyclyl optionally substituted with 1-3 groups independently selected from:
      N(R^{N})₂,
      C₁-C₉ alkyl optionally substituted with 1-4 groups independently selected from:
         oxo,
         N(R^{N})₂,
         C₁-C₆ alkoxy optionally substituted with 1-3 groups independently selected from C₆-C₁₀ aryl,
         C₆-C₁₀ aryl optionally substituted with 1-3 groups independently selected from hydroxyl, halogen, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, N(R^{N})₂, C₁-C₆ alkyl, C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₆-C₁₀ aryl, and
      3- to 10-membered heterocyclyl.
25. A compound of Formula Ia:

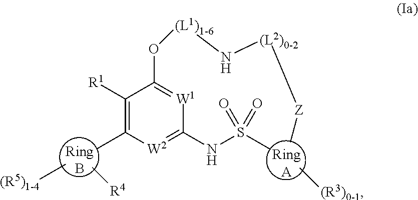

a tautomer thereof, a deuterated derivative of the compound or tautomer, or a pharmaceutically acceptable salt of any of the foregoing, wherein Ring A, Ring B, W¹, W², Z, L¹, L², R³, R⁴, and R⁵ are defined as according to embodiment 1.
26. The compound, tautomer, deuterated derivative, or pharmaceutically acceptable salt according to embodiment 25, wherein Ring A is selected from selected from C₆-C₁₀ aryl, 3- to 10-membered heterocyclyl, and 5- to 10-membered heteroaryl.
27. The compound, tautomer, deuterated derivative, or pharmaceutically acceptable salt according to embodiment 25 or 26, wherein Ring A is selected from phenyl, pyrazolyl, pyridyl, piperidinyl, and isoxazolyl.
28. The compound, tautomer, deuterated derivative, or pharmaceutically acceptable salt according to any one of embodiments 25 to 27, wherein Ring A is phenyl. 29. The compound, tautomer, deuterated derivative, or pharmaceutically acceptable salt according to any one of embodiments 25 to 28, wherein Ring B is selected from C₆-C₁₀ aryl, C₃-C₁₀ cycloalkyl, and 5- to 10-membered heteroaryl.
30. The compound, tautomer, deuterated derivative, or pharmaceutically acceptable salt according to any one of embodiments 25 to 29, wherein Ring B is selected from phenyl, napthyl, pyridinyl, pyrimidinyl, pyrazoyl, cyclopropyl, cyclobutyl, cyclohexyl, cyclohexenyl, pyrrolidinyl, 3,4-dihydro-2H-pyranyl, 3,6-dihydro-2H-pyranyl, cyclopentenyl, norbornenyl, and tetrahydropyranyl.
31. The compound, tautomer, deuterated derivative, or pharmaceutically acceptable salt according to any one of embodiments 25 to 30, wherein Ring B is phenyl.
32. The compound, tautomer, deuterated derivative, or pharmaceutically acceptable salt according to any one of embodiments 25 to 31, wherein W¹ is N and W² is N.
33. The compound, tautomer, deuterated derivative, or pharmaceutically acceptable salt according to any one of embodiments 25 to 32, wherein W¹ is CH and W² is N.
34. The compound, tautomer, deuterated derivative, or pharmaceutically acceptable salt according to any one of embodiments 25 to 33, wherein Z is selected from NR^{ZN} and C(R^{ZC})₂, provided that when L² is absent, Z is C(R^{ZC})₂
35. The compound, tautomer, deuterated derivative, or pharmaceutically acceptable salt according to any one of embodiments 25 to 34, wherein Ring C is a phenyl optionally substituted with 1-3 groups independently selected from:
   halogen,
   C₁-C₆ alkyl, and
   N(R^{N})₂.
36. The compound, tautomer, deuterated derivative, or pharmaceutically acceptable salt according to any one of embodiments 25 to 35, wherein each R³ is independently selected from:
   C₁-C₆ alkyl,
   C₃-C₁₀ cycloalkyl,
   C₆-C₁₀ aryl optionally substituted with 1-3 groups independently selected from C₁-C₆ alkyl, and
   3- to 10-membered heterocyclyl.
37. The compound, tautomer, deuterated derivative, or pharmaceutically acceptable salt according to any one of embodiments 25 to 36, wherein each R³ is independently selected from methyl, cyclopropyl, cyclohezyl, cyclohexenyl, tetrahyropyranyl, and 4-(tert-butyl)phenyl.
38. The compound, tautomer, deuterated derivative, or pharmaceutically acceptable salt according to any one of embodiments 25 to 35, wherein R³ is absent.
39. The compound, tautomer, deuterated derivative, or pharmaceutically acceptable salt according to any one of embodiments 25 to 38, wherein R⁴ is selected from hydrogen and methyl.
40. The compound, tautomer, deuterated derivative, or pharmaceutically acceptable salt according to any one of embodiments 25 to 39, wherein R⁴ is methyl.
41. The compound, tautomer, deuterated derivative, or pharmaceutically acceptable salt according to any one of embodiments 25 to 40, wherein each R⁵ is independently selected from:
   hydrogen,
   halogen,
   C₁-C₆ alkyl optionally substituted with 1-3 groups independently selected from:
      hydroxyl,
      C₁-C₆ alkoxy optionally substituted with 1-3 groups independently selected from C₆-C₁₀ aryl,
      C₃-C₁₀ cycloalkyl, and
      —(O)₀₋₁—(C₆-C₁₀ aryl) optionally substituted with 1-3 groups independently selected from C₁-C₆ alkyl and C₁-C₆ alkoxy,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl, and
   C₆-C₁₀ aryl.
42. The compound, tautomer, deuterated derivative, or pharmaceutically acceptable salt according to any one of embodiments 25 to 41, wherein R^{ZN} is hydrogen.
43. The compound, tautomer, deuterated derivative, or pharmaceutically acceptable salt according to any one of embodiments 25 to 42, wherein R^{ZC} is hydrogen, or two R^{ZC} are taken together to form an oxo group.
44. The compound, tautomer, deuterated derivative, or pharmaceutically acceptable salt according to any one of embodiments 25 to 43, wherein each R^{L1} is independently selected from:
   hydrogen,
   C₁-C₉ alkyl optionally substituted with 1-3 groups independently selected from:
      halogen,
      hydroxyl,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl,
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from hydroxyl, cyano, and SiMe₃,
      C₁-C₆ alkoxy optionally substituted with 1-3 groups independently selected from C₃-C₁₀ cycloalkyl (optionally substituted with 1-3 groups independently selected from C₁-C₆ fluoroalkyl), and C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl, and
      3- to 10-membered heterocyclyl optionally substituted with 1-3 groups independently selected from C₁-C₆ alkyl,
   3- to 10-membered heterocyclyl optionally substituted with 1-3 groups independently selected from C₁-C₆ alkyl (optionally substituted with 1-3 groups independently selected from oxo and C₁-C₆ alkoxy), and
   5- to 10-membered heteroaryl optionally substituted with 1-3 groups independently selected from C₁-C₆ alkyl.
45. The compound, tautomer, deuterated derivative, or pharmaceutically acceptable salt according to any one of embodiments 25 to 44, wherein each R^{L2} is independently selected from hydrogen and R^{F}, or two R^{L2} on the same carbon atom are taken together to form an oxo group.
46. The compound, tautomer, deuterated derivative, or pharmaceutically acceptable salt according to any one of embodiments 25 to 45, wherein each R^{N} is independently selected from:
   hydrogen,
   C₁-C₈ alkyl optionally substituted with 1-3 groups independently selected from oxo, C₁-C₆ alkoxy, and C₆-C₁₀ aryl, and
   C₃-C₁₀ cycloalkyl,
47. The compound, tautomer, deuterated derivative, or pharmaceutically acceptable salt according to any one of embodiments 25 to 46, wherein when R^{F} is present, two R^{F} taken together with the atoms to which they are bonded form a group selected from:
   C₃-C₁₀ cycloalkyl,
   C₆-C₁₀ aryl,
   3- to 11-membered heterocyclyl optionally substituted with 1-3 groups independently selected from:
      N(R^{N})₂,
      C₁-C₉ alkyl optionally substituted with 1-4 groups independently selected from:
         oxo,
         N(R^{N})₂,
         C₁-C₆ alkoxy optionally substituted with 1-3 groups independently selected from C₆-C₁₀ aryl,
         C₆-C₁₀ aryl optionally substituted with 1-3 groups independently selected from hydroxyl, halogen, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, N(R^{N})₂, C₁-C₆ alkyl, C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₆-C₁₀ aryl, and
      3- to 10-membered heterocyclyl.
48. A compound of Formula IIa:

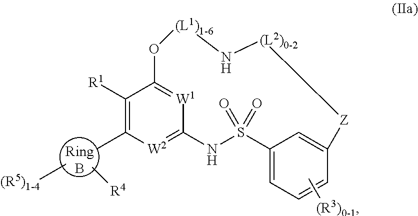

a tautomer thereof, a deuterated derivative of the compound or tautomer, or a pharmaceutically acceptable salt of any of the foregoing, wherein Ring B, W¹, W², Z, L¹, L², R³, R⁴, and R⁵ are defined as according to embodiment 1.
49. The compound, tautomer, deuterated derivative, or pharmaceutically acceptable salt according to embodiment 48, wherein Ring B is selected from C₆-C₁₀ aryl, C₃-C₁₀ cycloalkyl, and 5- to 10-membered heteroaryl.
50. The compound, tautomer, deuterated derivative, or pharmaceutically acceptable salt according to embodiment 48 or 49, wherein Ring B is selected from phenyl, napthyl, pyridinyl, pyrimidinyl, pyrazoyl, cyclopropyl, cyclobutyl, cyclohexyl, cyclohexenyl, pyrrolidinyl, 3,4-dihydro-2H-pyranyl, 3,6-dihydro-2H-pyranyl, cyclopentenyl, norbornenyl, and tetrahydropyranyl.
51. The compound, tautomer, deuterated derivative, or pharmaceutically acceptable salt according to any one of embodiments 48 to 50, wherein Ring B is phenyl.
52. The compound, tautomer, deuterated derivative, or pharmaceutically acceptable salt according to any one of embodiments 48 to 51, wherein W¹ is N and W² is N.
53. The compound, tautomer, deuterated derivative, or pharmaceutically acceptable salt according to any one of embodiments 48 to 52, wherein W¹ is CH and W² is N.
54. The compound, tautomer, deuterated derivative, or pharmaceutically acceptable salt according to any one of embodiments 48 to 53, wherein Z is selected from NR^{ZN} and C(R^{ZC})₂, provided that when L² is absent, Z is C(R^{ZC})₂
55. The compound, tautomer, deuterated derivative, or pharmaceutically acceptable salt according to any one of embodiments 48 to 54, wherein Ring C is a phenyl optionally substituted with 1-3 groups independently selected from:
   halogen,
   C₁-C₆ alkyl, and
   N(R^{N})₂.
56. The compound, tautomer, deuterated derivative, or pharmaceutically acceptable salt according to any one of embodiments 48 to 55, wherein each R³ is independently selected from:
   C₁-C₆ alkyl,
   C₃-C₁₀ cycloalkyl,
   C₆-C₁₀ aryl optionally substituted with 1-3 groups independently selected from C₁-C₆ alkyl, and
   3- to 10-membered heterocyclyl.
57. The compound, tautomer, deuterated derivative, or pharmaceutically acceptable salt according to any one of embodiments 48 to 56, wherein each R³ is independently selected from methyl, cyclopropyl, cyclohezyl, cyclohexenyl, tetrahyropyranyl, and 4-(tert-butyl)phenyl.
58. The compound, tautomer, deuterated derivative, or pharmaceutically acceptable salt according to any one of embodiments 48 to 55, wherein R³ is absent.
59. The compound, tautomer, deuterated derivative, or pharmaceutically acceptable salt according to any one of embodiments 48 to 58, wherein R⁴ is selected from hydrogen and methyl.
60. The compound, tautomer, deuterated derivative, or pharmaceutically acceptable salt according to any one of embodiments 48 to 59, wherein R⁴ is methyl.
61. The compound, tautomer, deuterated derivative, or pharmaceutically acceptable salt according to any one of embodiments 48 to 60, wherein each R⁵ is independently selected from:
   hydrogen,
   halogen,
   C₁-C₆ alkyl optionally substituted with 1-3 groups independently selected from:
      hydroxyl,
      C₁-C₆ alkoxy optionally substituted with 1-3 groups independently selected from C₆-C₁₀ aryl,
      C₃-C₁₀ cycloalkyl, and
      —(O)₀₋₁—(C₆-C₁₀ aryl) optionally substituted with 1-3 groups independently selected from C₁-C₆ alkyl and C₁-C₆ alkoxy,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl, and
   C₆-C₁₀ aryl.
62. The compound, tautomer, deuterated derivative, or pharmaceutically acceptable salt according to any one of embodiments 48 to 61, wherein R^{ZN} is hydrogen.
63. The compound, tautomer, deuterated derivative, or pharmaceutically acceptable salt according to any one of embodiments 48 to 62, wherein R^{ZC} is hydrogen, or two R^{ZC} are taken together to form an oxo group.
64. The compound, tautomer, deuterated derivative, or pharmaceutically acceptable salt according to any one of embodiments 48 to 63, wherein each R^{L1} is independently selected from:
   hydrogen,
   C₁-C₉ alkyl optionally substituted with 1-3 groups independently selected from:
      halogen,
      hydroxyl,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl,
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from hydroxyl, cyano, and SiMe₃,
      C₁-C₆ alkoxy optionally substituted with 1-3 groups independently selected from C₃-C₁₀ cycloalkyl (optionally substituted with 1-3 groups independently selected from C₁-C₆ fluoroalkyl), and C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl, and
      3- to 10-membered heterocyclyl optionally substituted with 1-3 groups independently selected from C₁-C₆ alkyl,
   3- to 10-membered heterocyclyl optionally substituted with 1-3 groups independently selected from C₁-C₆ alkyl (optionally substituted with 1-3 groups independently selected from oxo and C₁-C₆ alkoxy), and
   5- to 10-membered heteroaryl optionally substituted with 1-3 groups independently selected from C₁-C₆ alkyl.
65. The compound, tautomer, deuterated derivative, or pharmaceutically acceptable salt according to any one of embodiments 48 to 64, wherein each R^{L2} is independently selected from hydrogen and R^{F}, or two R^{L2} on the same carbon atom are taken together to form an oxo group.
66. The compound, tautomer, deuterated derivative, or pharmaceutically acceptable salt according to any one of embodiments 48 to 65, wherein each R^{N} is independently selected from:
   hydrogen,
   C₁-C₈ alkyl optionally substituted with 1-3 groups independently selected from oxo, C₁-C₆ alkoxy, and C₆-C₁₀ aryl, and
   C₃-C₁₀ cycloalkyl,
67. The compound, tautomer, deuterated derivative, or pharmaceutically acceptable salt according to any one of embodiments 48 to 66, wherein when R^{F} is present, two R^{F} taken together with the atoms to which they are bonded form a group selected from:
   C₃-C₁₀ cycloalkyl,
   C₆-C₁₀ aryl, and
   3- to 11-membered heterocyclyl optionally substituted with 1-3 groups independently selected from:
      N(R^{N})₂,
      C₁-C₉ alkyl optionally substituted with 1-4 groups independently selected from:
         oxo,
         N(R^{N})₂,
         C₁-C₆ alkoxy optionally substituted with 1-3 groups independently selected from C₆-C₁₀ aryl,
         C₆-C₁₀ aryl optionally substituted with 1-3 groups independently selected from hydroxyl, halogen, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, N(R^{N})₂, C₁-C₆ alkyl, C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₆-C₁₀ aryl, and
      3- to 10-membered heterocyclyl.
68. A compound of Formula IIb:

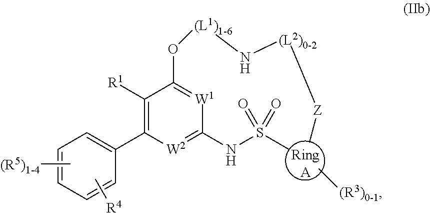

a tautomer thereof, a deuterated derivative of the compound or tautomer, or a pharmaceutically acceptable salt of any of the foregoing, wherein Ring A, W¹, W², Z, L¹, L², R³, R⁴, and R⁵ are defined as according to embodiment 1.
69. The compound, tautomer, deuterated derivative, or pharmaceutically acceptable salt according to embodiment 68, wherein Ring A is selected from selected from C₆-C₁₀ aryl, 3- to 10-membered heterocyclyl, and 5- to 10-membered heteroaryl.
70. The compound, tautomer, deuterated derivative, or pharmaceutically acceptable salt according to embodiment 68 or 69, wherein Ring A is selected from phenyl, pyrazolyl, pyridyl, piperidinyl, and isoxazolyl.
71. The compound, tautomer, deuterated derivative, or pharmaceutically acceptable salt according to any one of embodiments 68 to 70, wherein Ring A is phenyl.
72. The compound, tautomer, deuterated derivative, or pharmaceutically acceptable salt according to any one of embodiments 68 to 71, wherein W¹ is N and W² is N.
73. The compound, tautomer, deuterated derivative, or pharmaceutically acceptable salt according to any one of embodiments 68 to 72, wherein W¹ is CH and W² is N.
74. The compound, tautomer, deuterated derivative, or pharmaceutically acceptable salt according to any one of embodiments 68 to 73, wherein Z is selected from NR^{ZN} and C(R^{ZC})₂, provided that when L² is absent, Z is C(R^{ZC})₂
75. The compound, tautomer, deuterated derivative, or pharmaceutically acceptable salt according to any one of embodiments 68 to 74, wherein Ring C is a phenyl optionally substituted with 1-3 groups independently selected from:
   halogen,
   C₁-C₆ alkyl, and
   N(R^{N})₂.
76. The compound, tautomer, deuterated derivative, or pharmaceutically acceptable salt according to any one of embodiments 68 to 75, wherein each R³ is independently selected from:
   C₁-C₆ alkyl,
   C₃-C₁₀ cycloalkyl,
   C₆-C₁₀ aryl optionally substituted with 1-3 groups independently selected from C₁-C₆ alkyl, and
   3- to 10-membered heterocyclyl.
77. The compound, tautomer, deuterated derivative, or pharmaceutically acceptable salt according to any one of embodiments 68 to 76, wherein each R³ is independently selected from methyl, cyclopropyl, cyclohezyl, cyclohexenyl, tetrahyropyranyl, and 4-(tert-butyl)phenyl.
78. The compound, tautomer, deuterated derivative, or pharmaceutically acceptable salt according to any one of embodiments 68 to 75, wherein R³ is absent.
79. The compound, tautomer, deuterated derivative, or pharmaceutically acceptable salt according to any one of embodiments 68 to 78, wherein R⁴ is selected from hydrogen and methyl.
80. The compound, tautomer, deuterated derivative, or pharmaceutically acceptable salt according to any one of embodiments 68 to 79, wherein R⁴ is methyl.
81. The compound, tautomer, deuterated derivative, or pharmaceutically acceptable salt according to any one of embodiments 68 to 80, wherein each R⁵ is independently selected from:
   hydrogen,
   halogen,
   C₁-C₆ alkyl optionally substituted with 1-3 groups independently selected from:
      hydroxyl,
      C₁-C₆ alkoxy optionally substituted with 1-3 groups independently selected from C₆-C₁₀ aryl,
      C₃-C₁₀ cycloalkyl, and
      —(O)₀₋₁—(C₆-C₁₀ aryl) optionally substituted with 1-3 groups independently selected from C₁-C₆ alkyl and C₁-C₆ alkoxy,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl, and
   C₆-C₁₀ aryl.
82. The compound, tautomer, deuterated derivative, or pharmaceutically acceptable salt according to any one of embodiments 68 to 81, wherein R^{ZN} is hydrogen.
83. The compound, tautomer, deuterated derivative, or pharmaceutically acceptable salt according to any one of embodiments 68 to 82, wherein R^{ZC} is hydrogen, or two R^{ZC} are taken together to form an oxo group.
84. The compound, tautomer, deuterated derivative, or pharmaceutically acceptable salt according to any one of embodiments 68 to 83, wherein each R^{L1} is independently selected from:
   hydrogen,
   C₁-C₉ alkyl optionally substituted with 1-3 groups independently selected from:
      halogen,
      hydroxyl,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl,
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from hydroxyl, cyano, and SiMe₃,
      C₁-C₆ alkoxy optionally substituted with 1-3 groups independently selected from C₃-C₁₀ cycloalkyl (optionally substituted with 1-3 groups independently selected from C₁-C₆ fluoroalkyl), and C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl, and
      3- to 10-membered heterocyclyl optionally substituted with 1-3 groups independently selected from C₁-C₆ alkyl,
   3- to 10-membered heterocyclyl optionally substituted with 1-3 groups independently selected from C₁-C₆ alkyl (optionally substituted with 1-3 groups independently selected from oxo and C₁-C₆ alkoxy), and
   5- to 10-membered heteroaryl optionally substituted with 1-3 groups independently selected from C₁-C₆ alkyl.
85. The compound, tautomer, deuterated derivative, or pharmaceutically acceptable salt according to any one of embodiments 68 to 84, wherein each R^{L2} is independently selected from hydrogen and R^{F}, or two R^{L2} on the same carbon atom are taken together to form an oxo group.
86. The compound, tautomer, deuterated derivative, or pharmaceutically acceptable salt according to any one of embodiments 68 to 85, wherein each R^{N} is independently selected from:
   hydrogen,
   C₁-C₈ alkyl optionally substituted with 1-3 groups independently selected from oxo, C₁-C₆ alkoxy, and C₆-C₁₀ aryl, and
   C₃-C₁₀ cycloalkyl,
87. The compound, tautomer, deuterated derivative, or pharmaceutically acceptable salt according to any one of embodiments 68 to 86, wherein when R^{F} is present, two R^{F} taken together with the atoms to which they are bonded form a group selected from:
   C₃-C₁₀ cycloalkyl,
   C₆-C₁₀ aryl,
   3- to 11-membered heterocyclyl optionally substituted with 1-3 groups independently selected from:
      N(R^{N})₂,
      C₁-C₉ alkyl optionally substituted with 1-4 groups independently selected from:
         oxo,
         N(R^{N})₂,
         C₁-C₆ alkoxy optionally substituted with 1-3 groups independently selected from C₆-C₁₀ aryl,
         C₆-C₁₀ aryl optionally substituted with 1-3 groups independently selected from hydroxyl, halogen, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, N(R^{N})₂, C₁-C₆ alkyl, C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₆-C₁₀ aryl, and
      3- to 10-membered heterocyclyl.
88. A compound of Formula III can be depicted as:

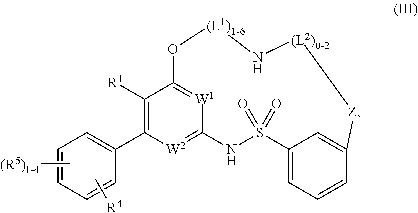

a tautomer thereof, a deuterated derivative of the compound or tautomer, or a pharmaceutically acceptable salt of any of the foregoing, wherein W¹, W², Z, L¹, L², R³, R⁴, and R⁵ are defined as according to embodiment 1.
89. The compound, tautomer, deuterated derivative, or pharmaceutically acceptable salt according to embodiment 88, wherein W¹ is N and W² is N.
90. The compound, tautomer, deuterated derivative, or pharmaceutically acceptable salt according to embodiment 88 or 89, wherein W¹ is CH and W² is N.
91. The compound, tautomer, deuterated derivative, or pharmaceutically acceptable salt according to any one of embodiments 88 to 90, wherein Z is selected from NR^{ZN} and C(R^{ZC})₂, provided that when L² is absent, Z is C(R^{ZC})₂
92. The compound, tautomer, deuterated derivative, or pharmaceutically acceptable salt according to any one of embodiments 88 to 91, wherein Ring C is a phenyl optionally substituted with 1-3 groups independently selected from:
   halogen,
   C₁-C₆ alkyl, and
   N(R^{N})₂.
93. The compound, tautomer, deuterated derivative, or pharmaceutically acceptable salt according to any one of embodiments 88 to 92, wherein R⁴ is selected from hydrogen and methyl.
94. The compound, tautomer, deuterated derivative, or pharmaceutically acceptable salt according to any one of embodiments 88 to 93, wherein R⁴ is methyl.
95. The compound, tautomer, deuterated derivative, or pharmaceutically acceptable salt according to any one of embodiments 88 to 94, wherein each R⁵ is independently selected from:
   hydrogen,
   halogen,
   C₁-C₆ alkyl optionally substituted with 1-3 groups independently selected from:
      hydroxyl,
      C₁-C₆ alkoxy optionally substituted with 1-3 groups independently selected from C₆-C₁₀ aryl,
      C₃-C₁₀ cycloalkyl, and
      —(O)₀₋₁—(C₆-C₁₀ aryl) optionally substituted with 1-3 groups independently selected from C₁-C₆ alkyl and C₁-C₆ alkoxy,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl, and
   C₆-C₁₀ aryl.
96. The compound, tautomer, deuterated derivative, or pharmaceutically acceptable salt according to any one of embodiments 88 to 95, wherein R^{ZN} is hydrogen.
97. The compound, tautomer, deuterated derivative, or pharmaceutically acceptable salt according to any one of embodiments 88 to 96, wherein R^{ZC} is hydrogen, or two R^{ZC} are taken together to form an oxo group.
98. The compound, tautomer, deuterated derivative, or pharmaceutically acceptable salt according to any one of embodiments 88 to 97, wherein each R^{L1} is independently selected from:
   hydrogen,
   C₁-C₉ alkyl optionally substituted with 1-3 groups independently selected from:
      halogen,
      hydroxyl,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl,
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from hydroxyl, cyano, and SiMe₃,
      C₁-C₆ alkoxy optionally substituted with 1-3 groups independently selected from C₃-C₁₀ cycloalkyl (optionally substituted with 1-3 groups independently selected from C₁-C₆ fluoroalkyl), and C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl, and
      3- to 10-membered heterocyclyl optionally substituted with 1-3 groups independently selected from C₁-C₆ alkyl,
   3- to 10-membered heterocyclyl optionally substituted with 1-3 groups independently selected from C₁-C₆ alkyl (optionally substituted with 1-3 groups independently selected from oxo and C₁-C₆ alkoxy), and
   5- to 10-membered heteroaryl optionally substituted with 1-3 groups independently selected from C₁-C₆ alkyl.
99. The compound, tautomer, deuterated derivative, or pharmaceutically acceptable salt according to any one of embodiments 88 to 98, wherein each R^{L2} is independently selected from hydrogen and R^{F}, or two R^{L2} on the same carbon atom are taken together to form an oxo group.
100. The compound, tautomer, deuterated derivative, or pharmaceutically acceptable salt according to any one of embodiments 88 to 99, wherein each R^{N} is independently selected from:
   hydrogen,
   C₁-C₈ alkyl optionally substituted with 1-3 groups independently selected from oxo, C₁-C₆ alkoxy, and C₆-C₁₀ aryl, and
   C₃-C₁₀ cycloalkyl,
101. The compound, tautomer, deuterated derivative, or pharmaceutically acceptable salt according to any one of embodiments 88 to 100, wherein when R^{F} is present, two R^{F} taken together with the atoms to which they are bonded form a group selected from:
   C₃-C₁₀ cycloalkyl,
   C₆-C₁₀ aryl,
   3- to 11-membered heterocyclyl optionally substituted with 1-3 groups independently selected from:
      N(R^{N})₂,
      C₁-C₉ alkyl optionally substituted with 1-4 groups independently selected from:
         oxo,
         N(R^{N})₂,
         C₁-C₆ alkoxy optionally substituted with 1-3 groups independently selected from C₆-C₁₀ aryl,
         C₆-C₁₀ aryl optionally substituted with 1-3 groups independently selected from hydroxyl, halogen, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, N(R^{N})₂, C₁-C₆ alkyl, C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₆-C₁₀ aryl, and
      3- to 10-membered heterocyclyl.
102. A compound of Formula IV:

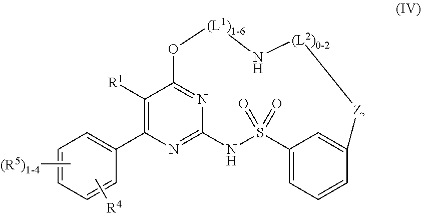

a tautomer thereof, a deuterated derivative of the compound or tautomer, or a pharmaceutically acceptable salt of any of the foregoing, wherein Z, L¹, L², R³, R⁴, and R⁵ are defined as according to embodiment 1.
103. The compound, tautomer, deuterated derivative, or pharmaceutically acceptable salt according to embodiment 102, wherein Z is selected from NR^{ZN} and C(R^{ZC})₂, provided that when L² is absent, Z is C(R^{ZC})₂
104. The compound, tautomer, deuterated derivative, or pharmaceutically acceptable salt according to embodiment 102 or 103, wherein Ring C is a phenyl optionally substituted with 1-3 groups independently selected from:
   halogen,
   C₁-C₆ alkyl, and
   N(R^{N})₂.
105. The compound, tautomer, deuterated derivative, or pharmaceutically acceptable salt according to any one of embodiments 102 to 104, wherein R⁴ is selected from hydrogen and methyl.
106. The compound, tautomer, deuterated derivative, or pharmaceutically acceptable salt according to any one of embodiments 102 to 105, wherein R⁴ is methyl.
107. The compound, tautomer, deuterated derivative, or pharmaceutically acceptable salt according to any one of embodiments 102 to 106, wherein each R⁵ is independently selected from:
   hydrogen,
   halogen,
   C₁-C₆ alkyl optionally substituted with 1-3 groups independently selected from:
      hydroxyl,
      C₁-C₆ alkoxy optionally substituted with 1-3 groups independently selected from C₆-C₁₀ aryl,
      C₃-C₁₀ cycloalkyl, and
      —(O)₀₋₁—(C₆-C₁₀ aryl) optionally substituted with 1-3 groups independently selected from C₁-C₆ alkyl and C₁-C₆ alkoxy,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl, and
   C₆-C₁₀ aryl.
108. The compound, tautomer, deuterated derivative, or pharmaceutically acceptable salt according to any one of embodiments 102 to 107, wherein R^{ZN} is hydrogen.
109. The compound, tautomer, deuterated derivative, or pharmaceutically acceptable salt according to any one of embodiments 102 to 108, wherein R^{ZC} is hydrogen, or two R^{ZC} are taken together to form an oxo group.
110. The compound, tautomer, deuterated derivative, or pharmaceutically acceptable salt according to any one of embodiments 102 to 109, wherein each R^{L1} is independently selected from:
   hydrogen,
   C₁-C₉ alkyl optionally substituted with 1-3 groups independently selected from:
      halogen,
      hydroxyl,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl,
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from hydroxyl, cyano, and SiMe₃,
      C₁-C₆ alkoxy optionally substituted with 1-3 groups independently selected from C₃-C₁₀ cycloalkyl (optionally substituted with 1-3 groups independently selected from C₁-C₆ fluoroalkyl), and C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl, and
      3- to 10-membered heterocyclyl optionally substituted with 1-3 groups independently selected from C₁-C₆ alkyl,
   3- to 10-membered heterocyclyl optionally substituted with 1-3 groups independently selected from C₁-C₆ alkyl (optionally substituted with 1-3 groups independently selected from oxo and C₁-C₆ alkoxy), and
   5- to 10-membered heteroaryl optionally substituted with 1-3 groups independently selected from C₁-C₆ alkyl.
111. The compound, tautomer, deuterated derivative, or pharmaceutically acceptable salt according to any one of embodiments 102 to 110, wherein each R^{L2} is independently selected from hydrogen and R^{F}, or two R^{L2} on the same carbon atom are taken together to form an oxo group.
112. The compound, tautomer, deuterated derivative, or pharmaceutically acceptable salt according to any one of embodiments 102 to 111, wherein each R^{N} is independently selected from:
   hydrogen,
   C₁-C₈ alkyl optionally substituted with 1-3 groups independently selected from oxo, C₁-C₆ alkoxy, and C₆-C₁₀ aryl, and
   C₃-C₁₀ cycloalkyl,
113. The compound, tautomer, deuterated derivative, or pharmaceutically acceptable salt according to any one of embodiments 102 to 112, wherein when R^{F} is present, two R^{F} taken together with the atoms to which they are bonded form a group selected from:
   C₃-C₁₀ cycloalkyl,
   C₆-C₁₀ aryl, and
   3- to 11-membered heterocyclyl optionally substituted with 1-3 groups independently selected from:
      N(R^{N})₂,
      C₁-C₉ alkyl optionally substituted with 1-4 groups independently selected from:
         oxo,
         N(R^{N})₂,
         C₁-C₆ alkoxy optionally substituted with 1-3 groups independently selected from C₆-C₁₀ aryl,
         C₆-C₁₀ aryl optionally substituted with 1-3 groups independently selected from hydroxyl, halogen, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, N(R^{N})₂, C₁-C₆ alkyl, C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₆-C₁₀ aryl, and
      3- to 10-membered heterocyclyl.
114. A compound of Formula V:

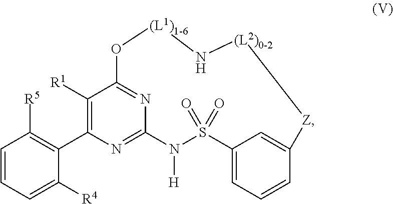

a tautomer thereof, a deuterated derivative of the compound or tautomer, or a pharmaceutically acceptable salt of any of the foregoing, wherein Z, L¹, L², R³, R⁴, and R⁵ are defined as according to embodiment 1.
115. The compound, tautomer, deuterated derivative, or pharmaceutically acceptable salt according to embodiment 114, wherein Z is selected from NR^{ZN} and C(R^{ZC})₂, provided that when L² is absent, Z is C(R^{ZC})₂
116. The compound, tautomer, deuterated derivative, or pharmaceutically acceptable salt according to embodiment 114 or 115, wherein Ring C is a phenyl optionally substituted with 1-3 groups independently selected from:
   halogen,
   C₁-C₆ alkyl, and
   N(R^{N})₂.
117. The compound, tautomer, deuterated derivative, or pharmaceutically acceptable salt according to any one of embodiments 114 to 116, wherein R⁴ is selected from hydrogen and methyl.
118. The compound, tautomer, deuterated derivative, or pharmaceutically acceptable salt according to any one of embodiments 114 to 117, wherein R⁴ is methyl.
119. The compound, tautomer, deuterated derivative, or pharmaceutically acceptable salt according to any one of embodiments 114 to 118, wherein each R⁵ is independently selected from:
   hydrogen,
   halogen,
   C₁-C₆ alkyl optionally substituted with 1-3 groups independently selected from:
      hydroxyl,
      C₁-C₆ alkoxy optionally substituted with 1-3 groups independently selected from C₆-C₁₀ aryl,
      C₃-C₁₀ cycloalkyl, and
      —(O)₀₋₁—(C₆-C₁₀ aryl) optionally substituted with 1-3 groups independently selected from C₁-C₆ alkyl and C₁-C₆ alkoxy,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl, and
   C₆-C₁₀ aryl.
120. The compound, tautomer, deuterated derivative, or pharmaceutically acceptable salt according to any one of embodiments 114 to 119, wherein R^{ZN} is hydrogen.
121. The compound, tautomer, deuterated derivative, or pharmaceutically acceptable salt according to any one of embodiments 114 to 120, wherein R^{ZC} is hydrogen, or two R^{ZC} are taken together to form an oxo group.
122. The compound, tautomer, deuterated derivative, or pharmaceutically acceptable salt according to any one of embodiments 114 to 121, wherein each R^{L1} is independently selected from:
   hydrogen,
   C₁-C₉ alkyl optionally substituted with 1-3 groups independently selected from:
      halogen,
      hydroxyl,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl,
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from hydroxyl, cyano, and SiMe₃,
      C₁-C₆ alkoxy optionally substituted with 1-3 groups independently selected from C₃-C₁₀ cycloalkyl (optionally substituted with 1-3 groups independently selected from C₁-C₆ fluoroalkyl), and C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl, and
      3- to 10-membered heterocyclyl optionally substituted with 1-3 groups independently selected from C₁-C₆ alkyl,
   3- to 10-membered heterocyclyl optionally substituted with 1-3 groups independently selected from C₁-C₆ alkyl (optionally substituted with 1-3 groups independently selected from oxo and C₁-C₆ alkoxy), and
   5- to 10-membered heteroaryl optionally substituted with 1-3 groups independently selected from C₁-C₆ alkyl.
123. The compound, tautomer, deuterated derivative, or pharmaceutically acceptable salt according to any one of embodiments 114 to 122, wherein each R¹² is independently selected from hydrogen and R^{F}, or two R^{L2} on the same carbon atom are taken together to form an oxo group.
124. The compound, tautomer, deuterated derivative, or pharmaceutically acceptable salt according to any one of embodiments 114 to 123, wherein each R^{N} is independently selected from:
   hydrogen,
   C₁-C₈ alkyl optionally substituted with 1-3 groups independently selected from oxo, C₁-C₆ alkoxy, and C₆-C₁₀ aryl, and
   C₃-C₁₀ cycloalkyl,
125. The compound, tautomer, deuterated derivative, or pharmaceutically acceptable salt according to any one of embodiments 114 to 124, wherein when R^{F} is present, two R^{F} taken together with the atoms to which they are bonded form a group selected from:
   C₃-C₁₀ cycloalkyl,
   C₆-C₁₀ aryl, and
   3- to 11-membered heterocyclyl optionally substituted with 1-3 groups independently selected from:
      N(R^{N})₂,
      C₁-C₉ alkyl optionally substituted with 1-4 groups independently selected from:
         oxo,
         N(R^{N})₂,
         C₁-C₆ alkoxy optionally substituted with 1-3 groups independently selected from C₆-C₁₀ aryl,
         C₆-C₁₀ aryl optionally substituted with 1-3 groups independently selected from hydroxyl, halogen, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, N(R^{N})₂, C₁-C₆ alkyl, C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₆-C₁₀ aryl, and
      3- to 10-membered heterocyclyl.
126. A compound of Formula VI:

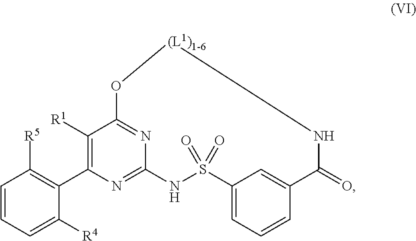

a tautomer thereof, a deuterated derivative of the compound or tautomer, or a pharmaceutically acceptable salt of any of the foregoing, wherein L¹, R³, R⁴, and R⁵ are defined as according to embodiment 1.
127. The compound, tautomer, deuterated derivative, or pharmaceutically acceptable salt according to embodiment 126, wherein Ring C is a phenyl optionally substituted with 1-3 groups independently selected from:
   halogen,
   C₁-C₆ alkyl, and
   N(R^{N})₂.
128. The compound, tautomer, deuterated derivative, or pharmaceutically acceptable salt according to embodiment 126 or 127, wherein R⁴ is selected from hydrogen and methyl.
129. The compound, tautomer, deuterated derivative, or pharmaceutically acceptable salt according to any one of embodiments 126 to 128, wherein R⁴ is methyl.
130. The compound, tautomer, deuterated derivative, or pharmaceutically acceptable salt according to any one of embodiments 126 to 129, wherein each R⁵ is independently selected from:
   hydrogen,
   halogen,
   C₁-C₆ alkyl optionally substituted with 1-3 groups independently selected from:
      hydroxyl,
      C₁-C₆ alkoxy optionally substituted with 1-3 groups independently selected from C₆-C₁₀ aryl,
      C₃-C₁₀ cycloalkyl, and
      —(O)₀₋₁—(C₆-C₁₀ aryl) optionally substituted with 1-3 groups independently selected from C₁-C₆ alkyl and C₁-C₆ alkoxy,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl, and
   C₆-C₁₀ aryl.
131. The compound, tautomer, deuterated derivative, or pharmaceutically acceptable salt according to any one of embodiments 126 to 130, wherein each R^{L1} is independently selected from:
   hydrogen,
   C₁-C₉ alkyl optionally substituted with 1-3 groups independently selected from:
      halogen,
      hydroxyl,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl,
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from hydroxyl, cyano, and SiMe₃,
      C₁-C₆ alkoxy optionally substituted with 1-3 groups independently selected from C₃-C₁₀ cycloalkyl (optionally substituted with 1-3 groups independently selected from C₁-C₆ fluoroalkyl), and C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl, and
      3- to 10-membered heterocyclyl optionally substituted with 1-3 groups independently selected from C₁-C₆ alkyl,
   3- to 10-membered heterocyclyl optionally substituted with 1-3 groups independently selected from C₁-C₆ alkyl (optionally substituted with 1-3 groups independently selected from oxo and C₁-C₆ alkoxy), and
   5- to 10-membered heteroaryl optionally substituted with 1-3 groups independently selected from C₁-C₆ alkyl.
132. The compound, tautomer, deuterated derivative, or pharmaceutically acceptable salt according to any one of embodiments 126 to 131, wherein each R^{N} is independently selected from:
   hydrogen,
   C₁-C₈ alkyl optionally substituted with 1-3 groups independently selected from oxo, C₁-C₆ alkoxy, and C₆-C₁₀ aryl, and
   C₃-C₁₀ cycloalkyl,
133. The compound, tautomer, deuterated derivative, or pharmaceutically acceptable salt according to any one of embodiments 126 to 132, wherein when R^{F} is present, two R^{F} taken together with the atoms to which they are bonded form a group selected from:
   C₃-C₁₀ cycloalkyl,
   C₆-C₁₀ aryl, and
   3- to 11-membered heterocyclyl optionally substituted with 1-3 groups independently selected from:
      N(R^{N})₂,
      C₁-C₉ alkyl optionally substituted with 1-4 groups independently selected from:
         oxo,
         N(R^{N})₂,
         C₁-C₆ alkoxy optionally substituted with 1-3 groups independently selected from C₆-C₁₀ aryl,
         C₆-C₁₀ aryl optionally substituted with 1-3 groups independently selected from hydroxyl, halogen, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, N(R^{N})₂, C₁-C₆ alkyl, C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N}, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₆-C₁₀ aryl, and
      3- to 10-membered heterocyclyl.
134. The compound, tautomer, deuterated derivative, or pharmaceutically acceptable salt according to any one of embodiments 1 to 133, selected from compounds of Formulae I, Ia, IIa, IIb, III, IV, V, and VI, and deuterated derivatives thereof and pharmaceutically acceptable salts of any of the foregoing.
135. The compound, tautomer, deuterated derivative, or pharmaceutically acceptable salt according to any one of embodiments 1 to 134, selected from Compounds 1-496 (Tables 3-5 and 7-10), deuterated derivatives thereof and pharmaceutically acceptable salts of any of the foregoing.
136. A pharmaceutical composition comprising the compound, tautomer, deuterated derivative, or pharmaceutically acceptable salt according to any one of embodiments 1 to
135, and a pharmaceutically acceptable carrier.
137. The pharmaceutical composition of embodiment 136, further comprising one or more additional therapeutic agents.
138. The pharmaceutical composition of embodiment 137, wherein the one or more additional therapeutic agent(s) is selected from mucolytic agents, bronchodilators, antibiotics, anti-infective agents, and anti-inflammatory agents.
139. The pharmaceutical composition of embodiment 137, wherein the one or more additional therapeutic agent(s) is an antibiotic selected from tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.
140. The pharmaceutical composition of embodiment 137, wherein the one or more additional therapeutic agent(s) is a CFTR modulator.
141. The pharmaceutical composition of embodiment 140, wherein the CFTR modulator is a potentiator.
142. The pharmaceutical composition of embodiment 140, wherein the CFTR modulator is a corrector.
143. The pharmaceutical composition of embodiment 137, wherein the one or more additional therapeutic agent(s) includes both a CFTR potentiator and a CFTR corrector.
144. The pharmaceutical composition of embodiment 141 or embodiment 143, wherein the CFTR potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.
145. The pharmaceutical composition of embodiment 142 or embodiment 143, wherein the CFTR corrector is selected from tezacaftor and lumacaftor.
146. The pharmaceutical composition of embodiment 144, wherein the composition comprises ivacaftor and tezacaftor.
147. The pharmaceutical composition of embodiment 144, wherein the composition comprises deutivacaftor and tezacaftor.
148. The pharmaceutical composition of embodiment 144, wherein the composition comprises (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and tezacaftor.
149. The pharmaceutical composition of embodiment 144, wherein the composition comprises ivacaftor and lumacaftor.
150. The pharmaceutical composition of embodiment 144, wherein the composition comprises deutivacaftor and lumacaftor.
151. The pharmaceutical composition of embodiment 144, wherein the composition comprises (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and lumacaftor.
152. A method of treating cystic fibrosis comprising administering to a patient in need thereof the compound, tautomer, deuterated derivative, or pharmaceutically acceptable salt according to any one of embodiments 1 to 135, or a pharmaceutical composition according to any one of embodiments 136 to 151.
153. The method of embodiment 152, further comprising administering to the patient one or more additional therapeutic agents prior to, concurrent with, or subsequent to the compound, tautomer, deuterated derivative, or pharmaceutically acceptable salt according to any one of embodiments 1 to 135, or the pharmaceutical composition according to any one of embodiments 136.
154. The method of embodiment 153, wherein the one or more additional therapeutic agents is (are) selected from one or more CFTR modulators.
155. The method of embodiment 154, wherein the one or more CFTR modulator(s) is a potentiator.
156. The method of embodiment 154, wherein the one or more CFTR modulator(s) is a corrector.
157. The method of embodiment 154, wherein the one or more CFTR modulators includes both a CFTR potentiator and a CFTR corrector.
158. The method of embodiment 155 or embodiment 157, wherein the CFTR potentiator is selected from ivacaftor, deutivacaftor, and deuterated derivatives and pharmaceutically acceptable salts of any of the foregoing.
159. The method of embodiment 155 or embodiment 157, wherein the CFTR potentiator is selected from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts of any of the foregoing.
160. The method of embodiment 156 or embodiment 157, wherein the CFTR corrector is selected from tezacaftor, lumacaftor, and deuterated derivatives and pharmaceutically acceptable salts thereof.
161. The method of embodiment 153, comprising administration of ivacaftor and tezacaftor.
162. The method of embodiment 153, comprising administration of deutivacaftor and tezacaftor.
163. The method of embodiment 153, comprising administration of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and tezacaftor.
164. The method of embodiment 153, comprising administration of ivacaftor and lumacaftor.
165. The method of embodiment 153, comprising administration of deutivacaftor and lumacaftor.
166. The method of embodiment 153, comprising administration of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and lumacaftor.
167. The compound, tautomer, deuterated derivative, or pharmaceutically acceptable salt according to any one of embodiments 1 to 135, or the pharmaceutical composition according to any one of embodiments 136 to 151 for use in the treatment of cystic fibrosis.
168. The compound, tautomer, deuterated derivative, or pharmaceutically acceptable salt according to any one of embodiments 1 to 135, or the pharmaceutical composition according to any one of embodiments 136 to 151 for use in the manufacture of a medicament for the treatment of cystic fibrosis.
169. A compound selected from Compounds 1-496, tautomers thereof, deuterated derivatives of those compounds and tautomers, and pharmaceutically acceptable salts of any of the foregoing.
170. A deuterated derivative of a compound selected from Compounds 1-496.
171. A pharmaceutically acceptable salt of a compound selected from Compounds 1-496.
172. A compound selected from Compounds 1-496.
173. A pharmaceutical composition comprising a compound selected from Compounds 1-496, tautomers thereof, deuterated derivatives of those compounds and tautomers, and pharmaceutically acceptable salts of any of the foregoing and a pharmaceutically acceptable carrier.
174. A pharmaceutical composition comprising a deuterated derivative of a compound selected from Compounds 1-496 and a pharmaceutically acceptable carrier.
175. A pharmaceutical composition comprising a pharmaceutically acceptable salt of a compound selected from Compounds 1-496 and a pharmaceutically acceptable carrier.
176. A pharmaceutical composition comprising a compound selected from Compounds 1-496 and a pharmaceutically acceptable carrier.
177. A pharmaceutical composition comprising (a) a compound selected from Compounds 1-496, tautomers thereof, deuterated derivatives of those compounds and tautomers, and pharmaceutically acceptable salts of any of the foregoing; (b) a CFTR potentiator; and (c) a pharmaceutically acceptable carrier.
178. A pharmaceutical composition composition comprising (a) a deuterated derivative of a compound selected from Compounds 1-496; (b) a CFTR potentiator; and (c) a pharmaceutically acceptable carrier.
179. A pharmaceutical comprising (a) a pharmaceutically acceptable salt of a compound selected from Compounds 1-496; (b) a CFTR potentiator; and (c) a pharmaceutically acceptable carrier.
180. A pharmaceutical composition comprising (a) a compound selected from Compounds 1-496; (b) a CFTR potentiator; and (c) a pharmaceutically acceptable carrier.
181. A pharmaceutical composition comprising (a) a compound selected from Compounds 1-496, tautomers thereof, deuterated derivatives of those compounds and tautomers, and pharmaceutically acceptable salts of any of the foregoing; (b) an additional CFTR corrector; and (c) a pharmaceutically acceptable carrier.
182. A pharmaceutical composition comprising (a) a deuterated derivative of a compound selected from Compounds 1-496; (b) an additional CFTR corrector; and (c) a pharmaceutically acceptable carrier.
183. A pharmaceutical composition comprising (a) a pharmaceutically acceptable salt of a compound selected from Compounds 1-496; (b) an additional CFTR corrector; and (c) a pharmaceutically acceptable carrier.
184. A pharmaceutical composition comprising (a) a compound selected from Compounds 1-496; (b) an additional CFTR corrector; and (c) a pharmaceutically acceptable carrier.
185. A pharmaceutical composition comprising (a) a compound selected from Compounds 1-496, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier.
186. A pharmaceutical composition comprising (a) a deuterated derivative of a compound selected from Compounds 1-496; (b) an additional CFTR corrector; (c) a CFTR potentiator; and (d) a pharmaceutically acceptable carrier.
187. A pharmaceutical composition comprising (a) a pharmaceutically acceptable salt of a compound selected from Compounds 1-496; (b) an additional CFTR corrector; (c) a CFTR potentiator; and (d) a pharmaceutically acceptable carrier.
188. A pharmaceutical composition comprising (a) a compound selected from Compounds 1-496; (b) an additional CFTR corrector; (c) a CFTR potentiator; and (d) a pharmaceutically acceptable carrier.
189. A compound selected from Compounds 1-496, tautomers thereof, deuterated derivatives of those compounds and tautomers, and pharmaceutically acceptable salts of any of the foregoing for use in a method of treating cystic fibrosis.
190. A deuterated derivative of a compound selected from Compounds 1-496 for use in a method of treating cystic fibrosis.
191. A pharmaceutically acceptable salt of a compound selected from Compounds 1-496 for use in a method of treating cystic fibrosis.
192. A compound selected from Compounds 1-496 for use in a method of treating cystic fibrosis.
193. A pharmaceutical composition comprising a compound selected from Compounds 1-496, tautomers thereof, deuterated derivatives of those compounds and tautomers, and pharmaceutically acceptable salts of any of the foregoing and a pharmaceutically acceptable carrier for use in a method of treating cystic fibrosis.
194. A pharmaceutical composition comprising a deuterated derivative of a compound selected from Compounds 1-496 and a pharmaceutically acceptable carrier for use in a method of treating cystic fibrosis.
195. A pharmaceutical composition comprising a pharmaceutically acceptable salt of a compound selected from Compounds 1-496 and a pharmaceutically acceptable carrier for use in a method of treating cystic fibrosis.
196. A pharmaceutical composition comprising a compound selected from Compounds 1-496 and a pharmaceutically acceptable carrier for use in a method of treating cystic fibrosis.
197. A pharmaceutical composition comprising (a) a compound selected from Compounds 1-496, tautomers thereof, deuterated derivatives of those compounds and tautomers, and pharmaceutically acceptable salts of any of the foregoing; (b) a CFTR potentiator; and (c) a pharmaceutically acceptable carrier for use in a method of treating cystic fibrosis.
198. A pharmaceutical comprising (a) a deuterated derivative of a compound selected from Compounds 1-496; (b) a CFTR potentiator; and (c) a pharmaceutically acceptable carrier for use in a method of treating cystic fibrosis.
199. A pharmaceutical composition comprising (a) a pharmaceutically acceptable salt of a compound selected from Compounds 1-496; (b) a CFTR potentiator; and (c) a pharmaceutically acceptable carrier for use in a method of treating cystic fibrosis.
200. A pharmaceutical composition comprising (a) a compound selected from Compounds 1-496; (b) a CFTR potentiator; and (c) a pharmaceutically acceptable carrier.
201. A pharmaceutical composition comprising (a) a compound selected from Compounds 1-496, tautomers thereof, deuterated derivatives of those compounds and tautomers, and pharmaceutically acceptable salts of any of the foregoing; (b) an additional CFTR corrector; and (c) a pharmaceutically acceptable carrier for use in a method of treating cystic fibrosis.
202. A pharmaceutical composition comprising (a) a deuterated derivative of a compound selected from Compounds 1-496; (b) an additional CFTR corrector; and (c) a pharmaceutically acceptable carrier for use in a method of treating cystic fibrosis.
203. A pharmaceutical composition comprising (a) a pharmaceutically acceptable salt of a compound selected from Compounds 1-496; (b) an additional CFTR corrector; and (c) a pharmaceutically acceptable carrier for use in a method of treating cystic fibrosis.
204. A pharmaceutical composition comprising (a) a compound selected from Compounds 1-496; (b) an additional CFTR corrector; and (c) a pharmaceutically acceptable carrier for use in a method of treating cystic fibrosis.
205. A pharmaceutical composition comprising (a) a compound selected from Compounds 1-496, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier for use in a method of treating cystic fibrosis.
206. A pharmaceutical composition comprising (a) a deuterated derivative of a compound selected from Compounds 1-496; (b) an additional CFTR corrector; (c) a CFTR potentiator; and (d) a pharmaceutically acceptable carrier for use in a method of treating cystic fibrosis.
207. A pharmaceutical composition comprising (a) a pharmaceutically acceptable salt of a compound selected from Compounds 1-496; (b) an additional CFTR corrector; (c) a CFTR potentiator; and (d) a pharmaceutically acceptable carrier for use in a method of treating cystic fibrosis.
208. A pharmaceutical composition comprising (a) a compound selected from Compounds 1-496; (b) an additional CFTR corrector; (c) a CFTR potentiator; and (d) a pharmaceutically acceptable carrier for use in a method of treating cystic fibrosis.

### EXAMPLES

### I. Abbreviation List

ACN: Acetonitrile
Boc anhydride ((Boc)₂O): Di-tert-butyl decarbonate
CDCl₃: Chloroform-d CDI: Carbonyl diimidazole
CDMT: 2-Chloro-4,6-dimethoxy-1,3,5-triazine
CH₂Cl₂: Dichloromethane
CH₃CN: Acetonitrile
COMU: (1-Cyano-2-ethoxy-2-oxoethylidenaminooxy)dimethylamino-morpholino-carbenium hexafluorophosphate
Cmpd: Compound
DABCO: 1,4-Diazabicyclo[2.2.2]octane
DBU: 1,8-Diazabicyclo(5.4.0)undec-7-ene
DCE: 1,2-Dichloroethane
DCM: Dichloromethane
DI: Deionized
DIAD: Diisopropyl azodicarboxylate
DIEA: (DIPEA, DiPEA): N,N-diisopropylethylamine
DMA: N,N-Dimethylacetamide
DMAP: 4-Dimethylaminopyridine
DMF: N,N-Dimethylformamide
DMSO: Dimethyl sulfoxide
DMP: Dess-Martin periodinane
EA: Ethyl acetate
EDC: 1-Ethyl-3-(3-dimethylaminopropyl)carbodiimide
ELSD: Evaporative light scattering detector
ESI-MS: Electrospray ionization mass spectrometry
diethylether: Diethyl ether
EtOAc: Ethyl acetate
EtOH: Ethanol
GC: Gas chromatography
Grubbs 1^{st} Generation catalyst: Dichloro(benzylidene)bis(tricyclohexylphosphine)ruthenium(II)
Grubbs 2^{nd} Generation catalyst: [1,3-Bis(2,4,6-trimethylphenyl)imidazolidin-2-ylidene]-dichloro-[(2-isopropoxyphenyl)methylene]ruthenium
HATU: 1-[Bis(dimethylamino)methylene]-1H-1,2,3-triazolo[4,5-b]pyridinium 3-oxid hexafluorophosphate
HPLC: High-performance liquid chromatography
Hoveyda-Grubbs 2^{nd} Generation catalyst: (1,3-LCis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)
IPA: Isopropanol
KHSO₄: Potassium bisulfate
LC: Liquid chromatography
LCMS: Liquid chromatography mass spectrometry
LCMS Met.: LCMS method
LCMS Rt: LCMS retention time
LDA: Lithium diisopropylamide
LiOH: Lithium hydroxide
MeCN: Acetonitrile
MeOH: Methanol
MeTHF or 2-MeTHF: 2-Methyltetrahydrofuran
MgSO₄: Magnesium sulfate
MTBE: Methyl tert-butyl ether
NaHCO₃: Sodium bicarbonate
NaOH: Sodium hydroxide
NMP: N-Methyl-2-pyrrolidone
NMM: N-Methylmorpholine
Pd/C: Palladium on carbon
Pd₂(dba)₃: Tris(dibenzylideneacetone)dipalladium(O)
Pd(dppf)Cl₂: [1,1′-Bis(diphenylphosphino)ferrocene]dichloropalladium(II)
Pd(OAc)₂: Palladium(II) acetate
PTFE: Polytetrafluoroethylene
rt, RT: Room temperature
RuPhos: 2-Dicyclohexylphosphino-2′,6′-diisopropoxybiphenyl
SFC: Supercritical fluid chromatography
TBAI: Tetrabutylammonium iodide
TEA: Triethylamine
TFA: Trifluoroacetic acid
THF: Tetrahydrofuran
TLC: Thin layer chromatography
TMS: Trimethylsilyl
TMSCl: Trimethylsilyl chloride
T3P: Propanephosphonic acid anhydride
UPLC: Ultra Performance Liquid Chromatography
XANTPHOS: 4,5-Bis(diphenylphosphino)-9,9-dimethylxanthene
XPhos: 2-Dicyclohexylphosphino-2′,4′,6′-triisopropylbiphenyl

### II. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. 19F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before fourier transform. 19F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H₂ carrier gas), at an injection temperature of 220° C. and an oven temperature of 120° C., 15 minutes.

### III. General UPLC/HPLC/GC Analytical Methods

LC method A: Analytical reverse phase UPLC using an Acquity UPLC BEH Cis column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method C: Kinetex C₁₈ 4.6×50 mm 2.6 μm. Temp: 45° C., Flow: 2.0 mL/min, Run Time: 3 min. Mobile phase: Initial 95% water (0.1% formic acid) and 5% acetonitrile (0.1% formic acid) linear gradient to 95% acetonitrile (0.1% formic acid) for 2.0 min then hold at 95% acetonitrile (0.1% formic acid) for 1.0 min.

LC method D: Acquity UPLC BEH C₁₈ column (30×2.1 mm, 1.7 m particle) made by Waters (pn: 186002349), and a dual gradient run from 1-99% mobile phase B over 1.0 minute. Mobile phase A=H₂O (0.05% CF₃CO₂H). Mobile phase B=CH₃CN (0.035% CF₃CO₂H). Flow rate=1.5 mL/min, injection volume=1.5 μL, and column temperature=60° C.

LC method I: Acquity UPLC BEH C₁₈ column (50×2.1 mm, 1.7 m particle) made by Waters (pn:186002350), and a dual gradient run from 1-99% mobile phase B over 5.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method J: Reverse phase UPLC using an Acquity UPLC BEH C₁₈ column (50×2.1 mm, 1.7 m particle) made by Waters (pn: 186002350), and a dual gradient run from 1-99% mobile phase B over 2.9 minutes. Mobile phase A=H₂O (0.05% NH₄HCO₂). Mobile phase B=CH₃CN. Flow rate=1.2 mL/min, injection volume=1.5 μL, and column temperature=60° C.

LC method K: Kinetex Polar C₁₈ 3.0×50 mm 2.6 μm, 3 min, 5-95% ACN in H₂O (0.1% Formic Acid) 1.2 ml/min.

LC method Q: Reversed phase UPLC using an Acquity UPLC BEH C₁₈ column (50×2.1 mm, 1.7 m particle) made by Waters (pn: 186002350), and a dual gradient run from 30-99% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method S: Merckmillipore Chromolith SpeedROD C₁₈ column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

LC method T: Merckmillipore Chromolith SpeedROD C₁₈ column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

LC method U: Kinetex Polar C₁₈ 3.0×50 mm 2.6 μm, 6 min, 5-95% ACN in H₂O (0.1% Formic Acid) 1.2 mL/min.

LC method V: Acquity UPLC BEH C₁₈ column (50×2.1 mm, 1.7 m particle) made by Waters (pn: 186002350), and a dual gradient run from 1-30% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method W: water Cortex 2.7μ C₁₈(3.0 mm×50 mm), Temp: 55° C.; Flow: 1.2 mL/min; mobile phase: 100% water with 0.1% trifluoroacetic(TFA) acid then 100% acetonitrile with 0.1% TFA acid, grad:5% to 100% B over 4 min, with stay at 100% B for 0.5 min, equilibration to 5% B over 1.5 min.

LC method X: UPLC Luna Cis(2) 50×3 mm 3 μm. run: 2.5 min. Mobile phase: Initial 95% H₂O 0.1% FA/5% MeCN 0.1% FA, linear grad to 95% MeCN 0.1% FA over 1.3 min, hold 1.2 min 95% CH₃CN 0.1% FA, T: 45C, Flow: 1.5 mL/min

LC method Y: UPLC SunFire C₁₈ 75×4.6 mm 3.5 m, run: 6 min. Mobile phase conditions: Initial 95% H₂O+0.1% FA/5% CH₃CN+0.1% FA, linear gradient to 95% CH₃CN for 4 min, hold for 2 min at 95% CH₃CN. T:45° C., Flow:1.5 mL/min

### IV. Synthesis of Common Intermediates

### Example 1: Preparation of 3-[[4-Chloro-6-(2,6-dimethylphenyl)pyrimidin-2-yl]sulfamoyl]benzoic Acid

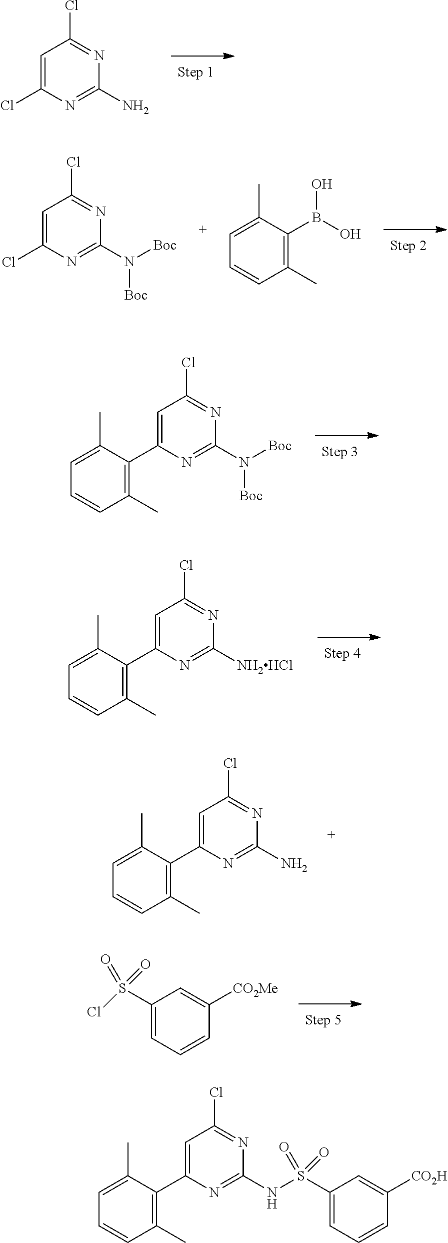

### Step 1: tert-Butyl N-tert-butoxycarbonyl-N-(4,6-dichloropyrimidin-2-yl)carbamate

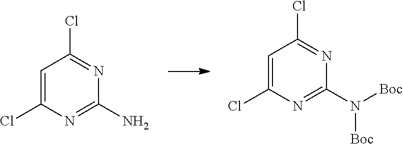 [SMILES: Nc1nc(Cl)cc(Cl)n1 ; CC(C)(C)OC(=O)N(C(=O)OC(C)(C)C)c1nc(Cl)cc(Cl)n1]

To a solution of 4,6-dichloropyrimidin-2-amine (300 g, 1.829 mol) in DCM (2.1 L) was added (BOC)₂O (838 g, 3.840 mol) followed by DMAP (5.6 g, 45.84 mmol). The mixture was stirred at ambient temperature for 6 h. Additional DMAP (5.6 g, 45.84 mmol) was added and the reaction was continued to stir at ambient temperature for 24 h. The mixture was diluted with water (2.1 L) and the organic phase separated. The organic phase was washed with water (2.1 L), 2.1 L of brine, dried over magnesium sulfate, filtered over Celite and concentrated in vacuo affording a light orange oil which had a silt in the slurry. The mixture was diluted with ˜500 mL of heptane and filtered using an M filter. The precipitate (SM) was washed with 250 mL of heptane. The filtrate was concentrated in vacuo affording a thick orange oil which was seeded with solid from a previous experiment and crystallized on standing, affording a light orange hard solid. tert-butyl N-tert-butoxycarbonyl-N-(4,6-dichloropyrimidin-2-yl)carbamate (645 g, 97%). ¹H NMR (400 MHz, DMSO-d₆) δ 8.07 (s, 1H), 1.44 (s, 18H). ESI-MS m/z calc. 363.07526, found 364.1 (M+1)⁺; Retention time: 2.12 minutes (LC method A).

### Step 2: tert-Butyl N-tert-butoxycarbonyl-N-[4-chloro-6-(2,6-dimethylphenyl)pyrimidin-2-yl]Carbamate

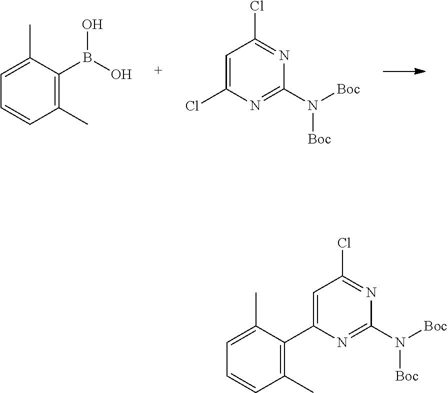 [SMILES: Cc1cccc(C)c1B(O)O ; CC(C)(C)OC(=O)N(C(=O)OC(C)(C)C)c1nc(Cl)cc(Cl)n1 ; Cc1cccc(C)c1-c1cc(Cl)nc(N(C(=O)OC(C)(C)C)C(=O)OC(C)(C)C)n1]

All solvents were degassed prior to use. To a slurry of tert-butyl N-tert-butoxycarbonyl-N-(4,6-dichloropyrimidin-2-yl)carbamate (88 g, 241.6 mmol), (2,6-dimethylphenyl)boronic acid (approximately 36.24 g, 241.6 mmol) and Cs₂CO₃ (approximately 196.8 g, 604.0 mmol) in DME (704 mL) and water (176 mL) were added. Pd(dppf)Cl₂ (approximately 8.839 g, 12.08 mmol) was added and the mixture was vigorously stirred under nitrogen at 80° C. (reflux) for 1 h (no SM remained). The reaction was cooled to ambient temperature and diluted with water (704 mL). The aqueous phase was separated and extracted with EtOAc (704 mL). The organic phase was washed with 700 mL of brine, dried over magnesium sulfate, filtered and concentrated in vacuo. The crude product was chromatographed on a 1500 g silica gel column eluting with 0-30% EtOAc/hexanes. The product fractions (eluted at 15% EtOAc) were combined and concentrated in vacuo affording the product as a clear oil which crystallized on standing. tert-butyl N-tert-butoxycarbonyl-N-[4-chloro-6-(2,6-dimethylphenyl)pyrimidin-2-yl]carbamate (81.3 g, 78%). ¹H NMR (400 MHz, DMSO-d₆) δ 7.88 (s, 1H), 7.30 (dd, J=8.2, 7.0 Hz, 1H), 7.21-7.16 (m, 2H), 2.03 (s, 6H), 1.38 (s, 18H). ESI-MS m/z calc. 433.17682, found 434.1 (M+1)+; Retention time: 2.32 minutes (LC method A).

### Step 3: 4-Chloro-6-(2,6-dimethylphenyl)pyrimidin-2-amine (hydrochloride salt)

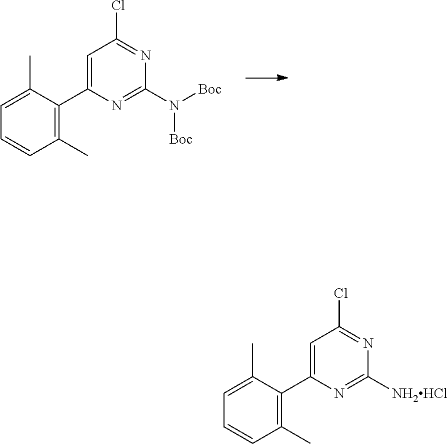

tert-Butyl N-tert-butoxycarbonyl-N-[4-chloro-6-(2,6-dimethylphenyl) pyrimidin-2-yl]carbamate (514.8 g, 915.9 mmol) was dissolved in dichloromethane (4 L). Hydrogen chloride in p-dioxane (1 L, 4 mol) was added and the mixture was stirred overnight at room temperature. The resulting precipitate was collected by vacuum filtration and dried in vacuo to obtain 4-chloro-6-(2,6-dimethylphenyl)pyrimidin-2-amine hydrochloride as a white solid (213.5 g, 82%). ¹H NMR (250 MHz, DMSO-d₆) δ 7.45-6.91 (m, 3H), 6.73 (s, 1H), 2.08 (s, 6H). ESI-MS m/z calc. 233.072, found 234.1 (M+1)⁺; Retention time: 2.1 minutes (LC Method C).

### Step 4: 4-Chloro-6-(2,6-dimethylphenyl)pyrimidin-2-amine

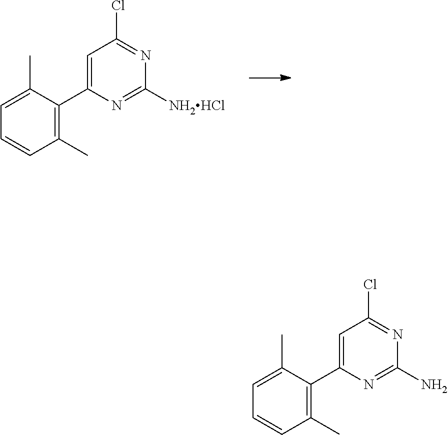

4-Chloro-6-(2,6-dimethylphenyl)pyrimidin-2-amine (hydrochloride salt) (166 g, 614.5 mmol) and 4-chloro-6-(2,6-dimethylphenyl)pyrimidin-2-amine (hydrochloride salt) (30 g, 111.0 mmol) were suspended in DCM (2.5 L), treated with NaOH (725 mL of 1 M, 725.0 mmol) and stirred at room temperature for 1 hour. The mixture was transferred into a separatory funnel and left standing over night. The DCM phase was separated and the aqueous phase with insoluble material was extracted twice more with DCM (2×500 mL). The combined brown DCM phases were stirred over magnesium sulfate and charcoal for 1 hour, filtered and the yellow solution concentrated to a volume of ˜500 mL. The solution was diluted with heptane (750 mL) and DCM was removed under reduced pressure at 60° C. to give a cream suspension. It was stirred at room temperature for 1 hour, filtered, washed with cold heptane and dried to give 4-chloro-6-(2,6-dimethylphenyl)pyrimidin-2-amine (157 g, 91%) as a cream solid. H NMR (400 MHz, DMSO-d₆) δ 7.28-7.14 (m, 3H), 7.10 (d, J 7.5 Hz, 2H), 6.63 (s, 1H), 2.06 (s, 6H). ESI-MS m/z calc. 233.07198, found 234.0 (M+1)⁺; Retention time: 1.45 minutes (LC method A).

### Step 5: 3-[[4-Chloro-6-(2,6-dimethylphenyl)pyrimidin-2-yl]sulfamoyl]benzoic Acid

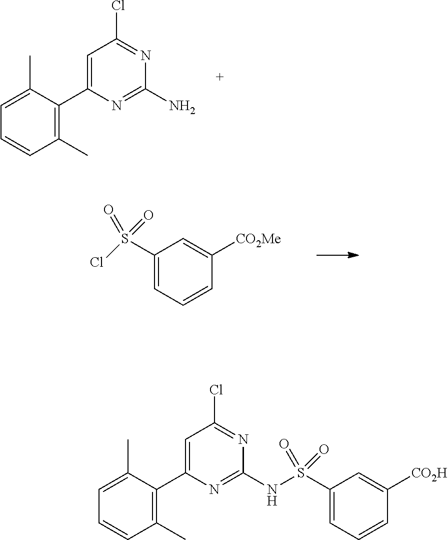 [SMILES: Cc1cccc(C)c1-c1cc(Cl)nc(N)n1 ; COC(=O)c1cccc(S(=O)(=O)Cl)c1 ; Cc1cccc(C)c1-c1cc(Cl)nc(NS(=O)(=O)c2cccc(C(=O)O)c2)n1]

4-Chloro-6-(2,6-dimethylphenyl)pyrimidin-2-amine (235 g, 985.5 mmol) was dissolved in MeTHF (2.3 L) and cooled in an ice bath under stirring and nitrogen. To the cold solution methyl 3-chlorosulfonylbenzoate (347 g, 1.479 mol) was added in one portion (seems slightly endothermic) and to the cold pale-yellow solution a solution of 2-methyl-butan-2-ol (Lithium salt) (875 mL of 3.1 M, 2.712 mol) (in heptane) was added dropwise over 1.25 hour (exothermic, internal temperature from 0 to 10° C.). The ice bath was removed and the greenish solution was stirred for 4 hours at room temperature. To the greenish solution cold HCl (2 L of 1.5 M, 3.000 mol) was added, the phases separated and the organic phase was washed once with water (1 L) and once with brine (500 mL). The aqueous phases were back extracted once with MeTHF (350 mL) and the organic phases were combined. This yellow MeTHF solution of methyl 3-[[4-chloro-6-(2,6-dimethylphenyl)pyrimidin-2-yl]sulfamoyl]benzoate (ESI-MS m/z calc. 431.07065, found 432.0 (M+1)⁺; Retention time: 1.81 minutes) was treated with NaOH (2.3 L of 2 M, 4.600 mol) and stirred at room temperature for 1 hour. The phases were separated and the NaOH phase was washed twice with MeTHF (2×500 mL) and the combined organic phases were extracted once with 2M NaOH (1×250 mL). The combined NaOH phases were combined, stirred in an ice bath and slowly acidified by addition of HCl (416 mL of 36% w/w, 4.929 mol) while keeping the internal temperature between 10 and 20° C. At the end of the addition (pH ˜5-6) the final pH was adjusted to 2-3 by addition of solid citric acid. The formed yellow tacky suspension was stirred at room temperature overnight to give a cream crisp suspension. The solid was collected by filtration, washed with plenty of water and sucked dry for 3 hours. The solid was dried under reduced pressure with a nitrogen leak at 45-50° C. for 120 hours 3-[[4-chloro-6-(2,6-dimethylphenyl)pyrimidin-2-yl]sulfamoyl]benzoic acid (395 g, 96%) was isolated as an off-white solid. ¹H NMR (400 MHz, DMSO-d₆) δ 13.44 (s, 1H), 12.46 (s, 1H), 8.48-8.39 (m, 1H), 8.25-8.15 (m, 1H), 8.15-8.08 (m, 1H), 7.68 (t, J 7.8 Hz, 1H), 7.31 (s, 1H), 7.28-7.18 (m, 1H), 7.10 (d, J 7.6 Hz, 2H), 1.84 (s, 6H). ESI-MS m/z calc. 417.055, found 418.0 (M+1)⁺; Retention time: 1.56 minutes. (LC method A).

### Example 2: Preparation of-[[4-[(2R)-2-(tert-Butoxycarbonylamino)-4-methyl-pentoxy]-6-(2,6-dimethylphenyl)pyrimidin-2-yl]sulfamoyl]benzoic Acid

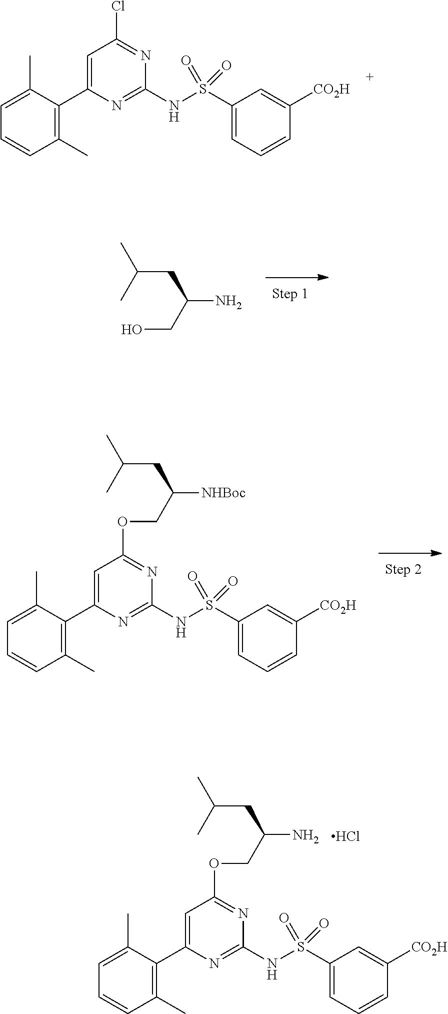

### Step 1: 3-[[4-[(2R)-2-(tert-Butoxycarbonylamino)-4-methyl-pentoxy]-6-(2,6-dimethylphenyl)pyrimidin-2-yl]sulfamoyl]benzoic Acid

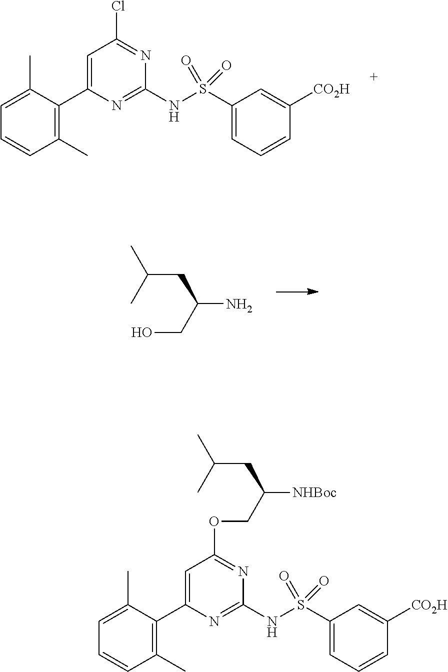

To a stirring solution of (2R)-2-amino-4-methyl-pentan-1-ol (12.419 g, 105.97 mmol) in anhydrous THE (200 mL) at room temperature under nitrogen was added sodium tert-butoxide (15.276 g, 158.95 mmol). The reaction mixture was stirred for 10 minutes and 3-[[4-chloro-6-(2,6-dimethylphenyl)pyrimidin-2-yl]sulfamoyl]benzoic acid (22.14 g, 52.983 mmol) was added. The reaction mixture was placed on a water bath preheated to 60° C. and stirred for 20 minutes. After cooling to room temperature, di-tert-butyl dicarbonate (69.381 g, 317.90 mmol) was added and the reaction mixture was stirred for 3 hours. The reaction was quenched with saturated aqueous ammonium chloride (150 mL). Volatiles were removed under vacuum and the aqueous layer was acidified to pH ˜3 with 10% aqueous citric acid. The product was extracted with ethyl acetate (3×200 mL). The combined organic layers were washed with brine (80 mL), dried over anhydrous sodium sulfate and concentrated to a residual volume of ˜250 mL. The product was precipitated out into excess hexanes (750 mL) and collected by vacuum filtration. The obtained white solid was re-purified by silica gel chromatography using 0-40% acetone (0.15% acetic acid buffer) gradient in hexanes (0.15% acetic acid buffer) to afford 3-[[4-[(2R)-2-(tert-butoxycarbonylamino)-4-methyl-pentoxy]-6-(2,6-dimethylphenyl)pyrimidin-2-yl]sulfamoyl]benzoic acid (20.73 g, 61%) as a white solid. ESI-MS m/z calc. 598.2461, found 599.4 (M+1)⁺; Retention time: 5.85 minutes (LC Method S).

### Step 2: 3-[[4-[(2R)-2-Amino-4-methyl-pentoxy]-6-(2,6-dimethylphenyl)pyrimidin-2-yl]sulfamoyl]benzoic Acid (Hydrochloride Salt)

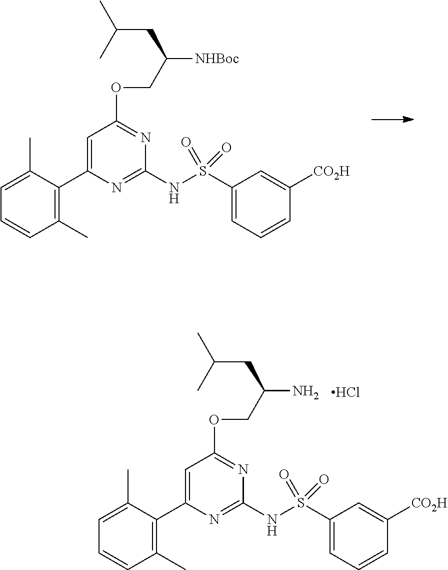 [SMILES: Cc1cccc(C)c1-c1cc(OC[C@@H](CC(C)C)NC(=O)OC(C)(C)C)nc(NS(=O)(=O)c2cccc(C(=O)O)c2)n1 ; Cc1cccc(C)c1-c1cc(OC[C@H](N)CC(C)C)nc(NS(=O)(=O)c2cccc(C(=O)O)c2)n1 ; Cl]

To a stirring solution of 3-[[4-[(2R)-2-(tert-butoxycarbonylamino)-4-methyl-pentoxy]-6-(2,6-dimethylphenyl)pyrimidin-2-yl]sulfamoyl]benzoic acid (20.73 g, 34.624 mmol) in DCM (200 mL) at room temperature was added HCl (87 mL of 4 M solution in 1,4-dioxane, 346.24 mmol). The reaction mixture was stirred for 2 hours. Volatiles were removed under vacuum and the obtained solid was triturated with diethyl ether (150 mL). After removal of the volatiles, the product was dried under vacuum to afford 3-[[4-[(2R)-2-amino-4-methyl-pentoxy]-6-(2,6-dimethylphenyl)pyrimidin-2-yl]sulfamoyl]benzoic acid (hydrochloride salt) (19.68 g, 100%) as a white solid. H NMR (250 MHz, DMSO-d₆) δ 8.56-8.27 (m, 4H), 8.14 (t, J 6.8 Hz, 2H), 7.70 (t, J 7.8 Hz, 1H), 7.34-7.18 (m, 1H), 7.17-7.02 (m, 2H), 6.31 (s, 1H), 4.42-4.23 (m, 1H), 4.23-4.06 (m, 1H), 3.5-3.4 (m, 1H, overlapped with water), 2.01 (s, 6H), 1.82-1.31 (m, 3H), 1.02-0.78 (m, 6H). ESI-MS m/z calc. 498.1937, found 499.3 (M+1)⁺; Retention time: 1.63 minutes (LC Method T).

### Example 3: Preparation of N-[4-chloro-6-(2,6-dimethylphenyl)pyrimidin-2-yl]-3-nitro-benzenesulfonamide

### Step 1: N-[4-Chloro-6-(2,6-dimethylphenyl)pyrimidin-2-yl]-3-nitro-benzenesulfonamide

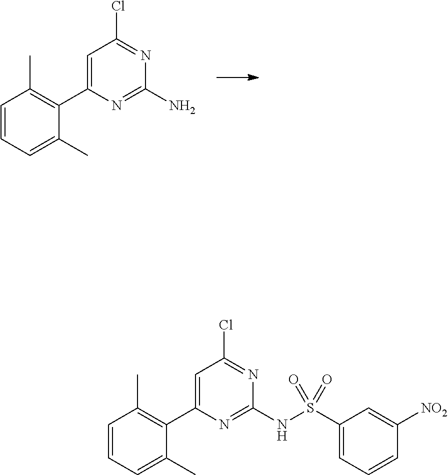 [SMILES: Cc1cccc(C)c1-c1cc(Cl)nc(N)n1 ; Cc1cccc(C)c1-c1cc(Cl)nc(NS(=O)(=O)c2cccc([N+](=O)[O-])c2)n1]

To a suspension of sodium hydride (60% in mineral oil) (4.87 g, 0.122 mol) in anhydrous tetrahydrofuran (30 mL) was added a solution of 4-chloro-6-(2,6-dimethylphenyl)pyrimidin-2-amine (8.13 g, 0.0348 mol) in anhydrous tetrahydrofuran (40 mL) dropwise at 0° C. The reaction mixture was stirred at room temperature for 30 minutes. A solution of 3-nitrobenzenesulfonyl chloride (11.57 g, 52.2 mmol) in anhydrous tetrahydrofuran (40 mL) was added to the reaction mixture dropwise at 0° C. The reaction was stirred at the same temperature for 1 hour. The reaction was quenched with a saturated aqueous solution of sodium bicarbonate (100 mL). The reaction solution was extracted with dichloromethane (3×100 mL). The combined organic layers were washed with water (100 mL), dried over anhydrous sodium sulfate, and then concentrated under vacuum. The residue was purified by silica gel column chromatography using 0 to 10% chloroform-ethyl acetate. The crude product was triturated with a solvent mixture of diethyl ether and hexane (1:5) to furnish N-[4-chloro-6-(2,6-dimethylphenyl)pyrimidin-2-yl]-3-nitro-benzenesulfonamide (5.98 g, 41%) as a white solid. ESI-MS m/z calc. 418.1, found 419.0 (M+1). Retention time: 5.73 minutes. ¹H NMR (250 MHz, CDCl₃) δ (ppm): 9.01 (s, 1H); 8.43 (t, J 10.5 Hz, 2H); 7.682 (t, J 7.8 Hz, 1H); 7.23 (m, 1H); 7.12 (d, J 7.5 Hz, 2H); 6.95 (s, 1H); 1.99 (s, 6H).

### Example 4: Preparation of N-[4-(2,6-dimethylphenyl)-6-methylsulfonyl-pyrimidin-2-yl]-3-nitro-benzenesulfonamide

### Step 1: N-[4-(2,6-Dimethylphenyl)-6-methylsulfonyl-pyrimidin-2-yl]-3-nitro-benzenesulfonamide

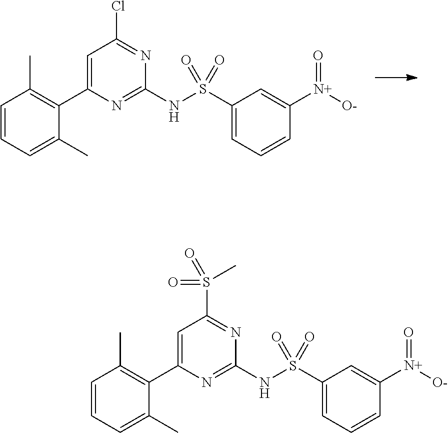 [SMILES: Cc1cccc(C)c1-c1cc(Cl)nc(NS(=O)(=O)c2cccc([N+](=O)[O-])c2)n1 ; Cc1cccc(C)c1-c1cc(S(C)(=O)=O)nc(NS(=O)(=O)c2cccc([N+](=O)[O-])c2)n1]

Stage 1: To a 250 mL round-bottomed flask were added N-[4-chloro-6-(2,6-dimethylphenyl)pyrimidin-2-yl]-3-nitro-benzenesulfonamide (14.14 g, 33.76 mmol), sodium thiomethoxide (5.86 g, 83.61 mmol) and NMP (130 mL). This solution was stirred at 100° C. for 3 h. The reaction mixture was then cooled to room temperature, quenched with 1 N HCl (300 mL), and extracted with ethyl acetate (3×300 mL). The combined organic extracts were washed with water (300 mL), 3% aqueous hydrogen peroxide solution (300 mL), water (300 mL) and saturated aqueous sodium chloride solution (300 mL), then dried over sodium sulfate, filtered, and evaporated in vacuo. This gave an orange foam (16.71 g, 115% crude product yield) that was carried onto the next reaction.

Stage 2: To a 250 mL round-bottomed flask containing the product from Stage 1, DCM (120 mL) was added, followed by m-CPBA (77% pure, 27.22 g, 121.5 mmol). This solution was stirred at room temperature for 90 min. The reaction mixture was quenched by transferring to a 1 L-Erlenmeyer flask containing DCM (400 mL) and solid Na₂S₂O₃ (41.15 g, 260.3 mmol). This mixture was stirred at room temperature for 1 h. The reaction mixture was diluted with DCM (300 mL), then washed with water (3×400 mL) and saturated aqueous sodium chloride solution (300 mL). The organic layer was then dried over sodium sulfate, filtered, and evaporated in vacuo. This solid was then partially dissolved in DCM (100 mL) and filtered in vacuo on a Buchner funnel to remove the m-chlorobenzoic acid waste (this was repeated three times). The remaining solution was then purified by silica gel chromatography (330 g of silica, 0 to 60% gradient of ethyl acetate/hexanes) to give N-[4-(2,6-dimethylphenyl)-6-methylsulfonyl-pyrimidin-2-yl]-3-nitro-benzenesulfonamide (5.881 g, 36%). ESI-MS m z calc. 462.06677, found 463.1 (M+1)⁺; Retention time: 1.6 minutes; LC method A.

### Example 5: Preparation of 3-[[4-[(2R)-2-amino-4,4-dimethyl-pentoxy]-6-(2,6-dimethylphenyl)pyrimidin-2-yl]sulfamoyl]benzoic Acid

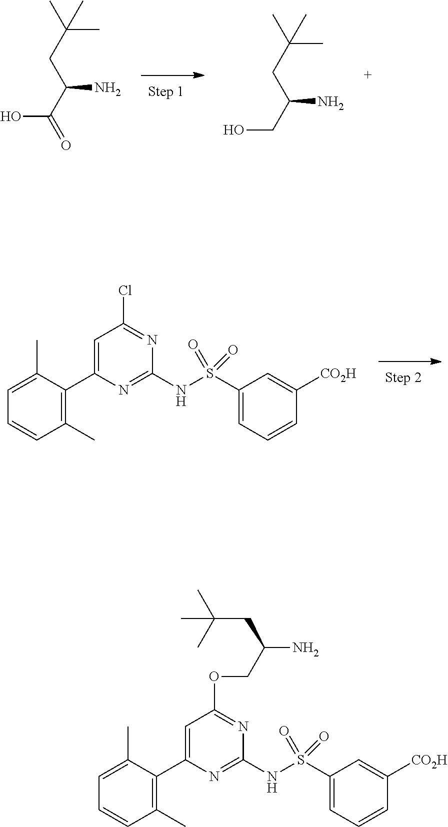

### Step 1: (2R)-2-Amino-4,4-dimethyl-pentan-1-ol

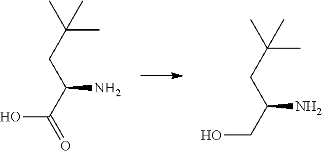

To a solution of (2R)-2-amino-4,4-dimethyl-pentanoic acid (15 g, 103.3 mmol) in THE (150 mL) at 0° C. was added borane-THF (260 mL of 1 M, 260.0 mmol) dropwise keeping the reaction temperature <10° C. The addition took approximately 30 min. The mixture was allowed to warm to ambient temperature and stirred for 22 h. The reaction was quenched with the slow addition of methanol (80 mL, 1.975 mol) and the solvent was removed in vacuo. The residue was co-evaporated 3× with methanol (200 mL, 4.937 mol) The crude residue was diluted with HCl (200 mL of 1 M, 200.0 mmol) and washed with 200 mL of MTBE. The aqueous phase was evaporated to remove residual organic solvent. The water was further removed in vacuo affording an off-white solid. The solid was further dried using an acetonitrile azeotrope. The solid was slurried in 200 mL of ACN and the precipitate collected using a M frit. The solid was air dried for 1 h, then in vacuo at 45° C. for 20 h to give (2R)-2-amino-4,4-dimethyl-pentan-1-ol (hydrochloride salt) (14.73 g, 85%). ¹H NMR (400 MHz, DMSO-d₆) δ 7.80 (s, 3H), 5.36 (t, J 5.1 Hz, 1H), 3.59 (dt, J 11.7, 4.1 Hz, 1H), 3.42-3.34 (m, 1H), 3.10 (dq, J 7.7, 3.8 Hz, 1H), 1.46 (dd, J 14.5, 7.1 Hz, 1H), 1.33 (dd, J 14.5, 3.5 Hz, 1H), 0.91 (s, 9H). ESI-MS m z calc. 131.13101, found 132.1 (M+1)⁺; Retention time: 0.51 minutes (LC method A).

### Step 2: 3-[[4-[(2R)-2-Amino-4,4-dimethyl-pentoxy]-6-(2,6-dimethylphenyl)pyrimidin-2-yl]sulfamoyl]benzoic Acid

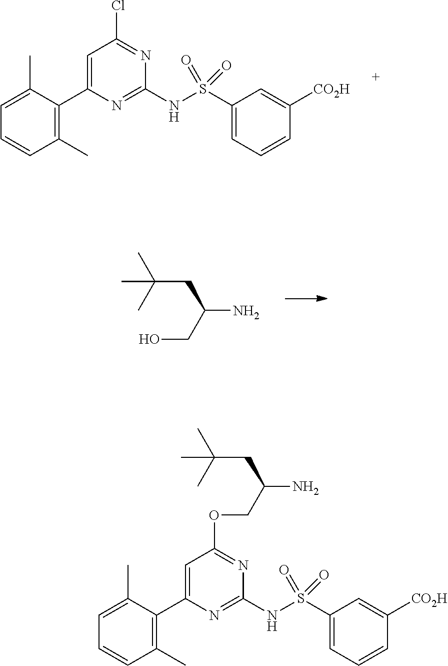

3-[[4-Chloro-6-(2,6-dimethylphenyl)pyrimidin-2-yl]sulfamoyl]benzoic acid (20 g, 47.862 mmol) was suspended in a mixture of 2-methyltetrahydrofuran (80 mL) and DMF (20 mL) and the solution was cooled to −5° C. Sodium tert-butoxide (23 g, 239.33 mmol) was then dissolved in 2-methyltetrahydrofuran (100 mL), cooled to 5° C. and added over 10 minutes, followed by (2R)-2-amino-4,4-dimethyl-pentan-1-ol (hydrochloride salt) (8.02 g, 47.830 mmol) the reaction was then warmed to 10° C. and stirred for 4 hours. It was then cooled to 0° C. and quenched by adding an aqueous solution of hydrochloric acid (2 M, 200 mL) over 10 minutes. The phases were separated, and the aqueous phase extracted with 2-methyltetrahydrofuran (200 mL). The organic phases were combined and washed with an aqueous solution of sodium chloride (15% w/w, 2×200 mL), dried over sodium sulfate (60 g), filtered and evaporated to dryness. The solid was then triturated using ethyl acetate (200 mL) for 16 hours, filtered, washed with ethyl acetate and dried in a vacuum oven at 50° C. for 20 hours to give 3-[[4-[(2R)-2-amino-4,4-dimethyl-pentoxy]-6-(2,6-dimethylphenyl)pyrimidin-2-yl]sulfamoyl]benzoic Acid (hydrochloride salt) (22.29 g, 80%). ¹H NMR (400 MHz, DMSO-d₆) δ 13.26 (br. s., 2H), 8.45 (t, J 1.6 Hz, 1H), 8.28-8.06 (m, 5H), 7.69 (t, J 7.8 Hz, 1H), 7.31-7.21 (m, 1H), 7.13 (d, J 7.6 Hz, 2H), 6.29 (br. s., 1H), 4.30 (dd, J 11.7, 2.7 Hz, 1H), 4.10 (dd, J 11.5, 7.1 Hz, 1H), 3.56 (br. s., 1H), 2.13-1.90 (s, 6H), 1.62-1.47 (m, 2H), 0.94 (s, 9H). ESI-MS m/z calc. 512.20935, found 513.0 (M+1)⁺; Retention time: 2.334 minutes; LC method U.

### Example 6: Preparation of 3-[[4-[(2R)-2-amino-5,5,5-trifluoro-4,4-dimethyl-pentoxy]-6-(2,6-dimethylphenyl)pyrimidin-2-yl]sulfamoyl]benzoic Acid

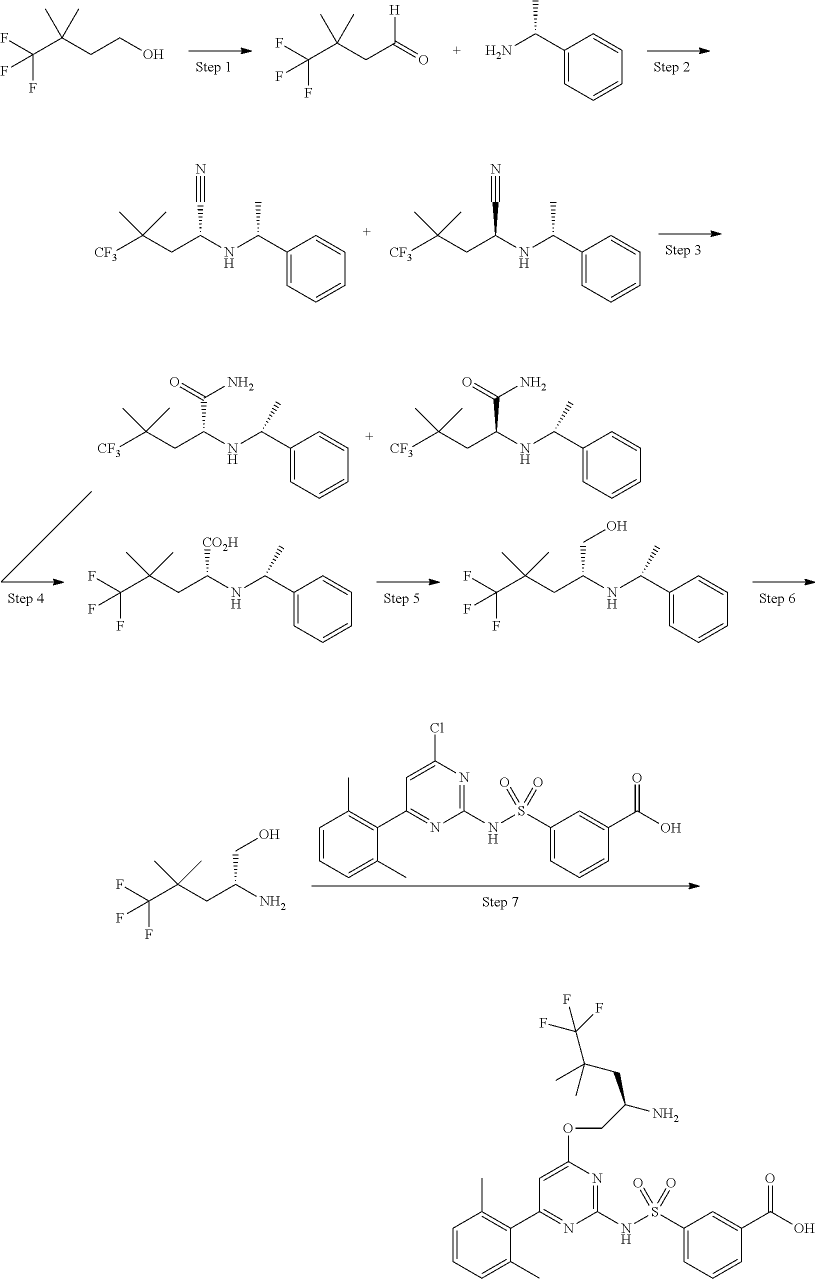

### Step 1: 4,4,4-Trifluoro-3,3-dimethyl-butanal

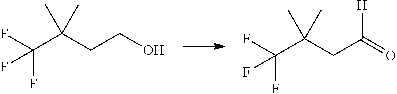

A 1 L three-neck flask was charged with 4,4,4-trifluoro-3,3-dimethyl-butan-1-ol (8.987 g, 57.555 mmol), DCM (63 mL), water (63 mL), NaBr (544 mg, 5.2870 mmol), sodium bicarbonate (12.32 g, 146.66 mmol) and TEMPO (92 mg, 0.5888 mmol). The mixture was cooled with ice-water bath. A aqueous solution of NaOCl (47 mL of 1.31 M, 61.570 mmol) was added dropwise over 2 h at 2.5-4.4° C. After the addition, the mixture was stirred for 10 min. The two layers was separated. The aqueous phase was extracted with DCM (2×15 mL). The combined organic layers were dried with sodium sulfate and filtered to give 113.7 g (about 80 mL) of crude product in DCM, which was used directly the next step. ¹H NMR (300 MHz, CDCl₃) δ 9.82-9.78 (m, 1H), 2.54 (d, J=2.6 Hz, 2H), 1.28 (s, 6H). ¹⁹F NMR (282 MHz, CDCl₃) δ-79.11 (s, 3F).

### Step 2: (2R)-5,5,5-Trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile

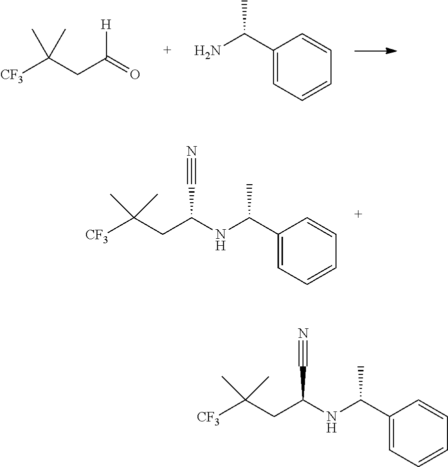

To a DCM (80 mL) solution of 4,4,4-trifluoro-3,3-dimethyl-butanal (113.7 g, 57.540 mmol) (purity about 7.8%) was added MeOH (110 mL). The mixture was cooled with ice-water bath. (1R)-1-phenylethanamine (8.46 g, 69.814 mmol) was added, followed by acetic acid (4.41 g, 73.436 mmol). The mixture was stirred at 0° C. for 10 min, then NaCN (3.56 g, 72.642 mmol) was added. The mixture was allowed to warm to rt slowly and stirred overnight. The reaction mixture was cooled to 0° C. and a solution of potassium carbonate (4 g) in water (20 mL) was added dropwise, followed by brine (40 mL). The mixture was extracted with DCM (2×100 mL). The organic layers were dried with sodium sulfate, filtered and concentrated. The residue was purified by flash chromatography (120 g silica gel, heptanes/EtOAc 0-30%) to afford a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 91%) as a colorless oil. ESI-MS m/z calc. 284.15002, found 285.2 (M+1)⁺; Retention time: 3.38 minutes; LC method U.

### Step 3: (2R)-5,5,5-Trifluoro-4,4-dimethyl-2-[[(1R)-1-phenylethyl]amino]pentanamide and (2S)-5,5,5-trifluoro-4,4-dimethyl-2-[[(1R)-1-phenylethyl]amino]pentanamide

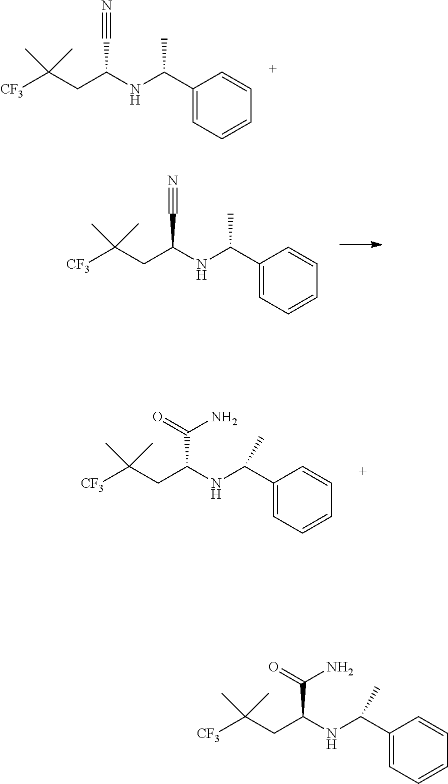 [SMILES: C[C@@H](N[C@@H](C#N)CC(C)(C)C(F)(F)F)c1ccccc1 ; C[C@@H](N[C@H](C#N)CC(C)(C)C(F)(F)F)c1ccccc1 ; C[C@@H](N[C@H](CC(C)(C)C(F)(F)F)C(N)=O)c1ccccc1 ; C[C@@H](N[C@@H](CC(C)(C)C(F)(F)F)C(N)=O)c1ccccc1]

To a solution of a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 52.300 mmol) in DCM (105 mL) was added sulfuric acid (56.3 g, 551.06 mmol). The mixture was stirred at rt overnight, poured on crude ice (200 g) and neutralized to pH 9 with 28% NH₃ in water (100 mL). The mixture was extracted with DCM (500 mL). The organic layer was dried with sodium sulfate, filtered and concentrated. The residue was purified by flash chromatography (330 g silica gel, heptanes/EtOAc 20-50%) to afford (2R)-5,5,5-trifluoro-4,4-dimethyl-2-[[(1R)-1-phenylethyl]amino]pentanamide (10.77 g, 68%) as a white solid. ¹H NMR (300 MHz, CDCl₃) δ 7.39-7.22 (m, 5H), 6.35 (br. s., 1H), 5.55 (br. s., 1H), 3.65 (q, J=6.5 Hz, 1H), 2.93 (dd, J=7.6, 3.8 Hz, 1H), 1.87 (dd, J=15.0, 3.8 Hz, 1H), 1.65-1.56 (m, 2H), 1.35 (d, J=6.5 Hz, 3H), 1.04 (s, 3H), 1.00 (s, 3H). ¹⁹F NMR (282 MHz, CDCl₃) δ-78.77 (s, 3F). 99.4% de by 19F NMR.

### Step 4: (2R)-5,5,5-Trifluoro-4,4-dimethyl-2-[[(1R)-1-phenylethyl]amino]pentanoic Acid

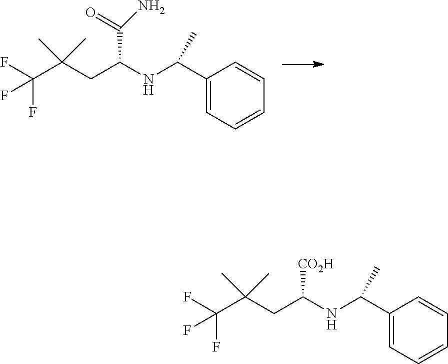 [SMILES: C[C@@H](N[C@H](CC(C)(C)C(F)(F)F)C(N)=O)c1ccccc1 ; C[C@@H](N[C@H](CC(C)(C)C(F)(F)F)C(=O)O)c1ccccc1]

To a solution of (2R)-5,5,5-trifluoro-4,4-dimethyl-2-[[(1R)-1-phenylethyl]amino]pentanamide (11.35 g, 37.541 mmol) in HOAc (50 mL) was added conc. HCl (65 mL of 11.8 M, 767.00 mmol), followed by water (50 mL). A white precipitate appeared. The mixture was heated at 100° C. for 66 h. More conc. HCl (40 mL of 11.8 M, 472.00 mmol) and HOAc (10 mL) were added. The mixture was stirred at 100° C. overnight. More HCl in water (20 mL of 6 M, 120.00 mmol) was added. After 7 h at 100° C., more HCl in water (20 mL of 6 M, 120.00 mmol) was added. The mixture was stirred at 100° C. overnight. It became a clear solution. More HCl in water (20 mL of 6 M, 120.00 mmol) was added. The mixture was stirred at 100° C. for 7 h, more HCl in water (20 mL of 6 M, 120.00 mmol) was added. The mixture was stirred at 100° C. overnight. The mixture was concentrated and co-evaporated with water (50 mL). The residue (17 g) was mixed with water (25 mL) at 50° C. for 20 min, cooled with ice-water bath for 20 min and filtered. The crude product was mixed with 1,4-dioxane (60 mL). The mixture was concentrated and dried on vacuum overnight to give (2R)-5,5,5-trifluoro-4,4-dimethyl-2-[[(1R)-1-phenylethyl]amino]pentanoic acid (hydrochloride salt) (13.04 g, 97%) as an off-white solid. ¹H NMR (300 MHz, DMSO-d₆) δ 10.09 (br. s., 1H), 7.54-7.31 (m, 5H), 7.29-7.05 (m, 1H), 4.07 (q, J=5.9 Hz, 1H), 3.16-2.98 (m, 1H), 2.08-1.83 (m, 2H), 1.49 (d, J=6.5 Hz, 3H), 0.99 (s, 3H), 0.92 (s, 3H). ¹⁹F NMR (282 MHz, DMSO-d₆) δ-78.28 (s, 3F). ESI-MS m/z calc. 303.14462, found 304.2 (M+1)⁺; Retention time: 1.98 minutes; LC method U.

### Step 5: (2R)-5,5,5-Trifluoro-4,4-dimethyl-2-[[(1R)-1-phenylethyl]amino]pentan-1-ol

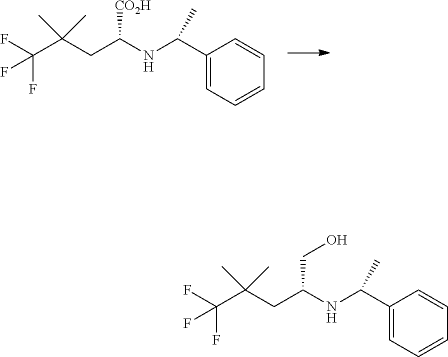 [SMILES: C[C@@H](N[C@H](CC(C)(C)C(F)(F)F)C(=O)O)c1ccccc1 ; C[C@@H](N[C@@H](CO)CC(C)(C)C(F)(F)F)c1ccccc1]

To a suspension of (2R)-5,5,5-trifluoro-4,4-dimethyl-2-[[(1R)-1-phenylethyl]amino]pentanoic acid (hydrochloride salt) (13.04 g, 36.267 mmol) in THE (200 mL) at 35° C. was added LAH in THF (100 mL of 1 M, 100.00 mmol) dropwise. The mixture was stirred at 40° C. for 2 h, cooled to 10° C. with ice-water bath and diluted with THE (200 mL). A mixture of water (3.8 g) and THE (50 mL) was added dropwise, followed by 25% aqueous NaOH (3.8 g) and water (10 g). The resulting mixture was stirred at rt for 30 min and at 50° C. for 1 h, filtered and washed with warm THF. The filtrate was concentrated to give 12.02 g of product (free amine) as a colorless oil. ¹H NMR (300 MHz, CDCl₃) δ 7.37-7.24 (m, 5H), 3.82 (q, J=6.5 Hz, 1H), 3.72-3.67 (m, 1H), 3.21 (dd, J=10.6, 4.7 Hz, 1H), 2.67 (quin, J=4.6 Hz, 1H), 1.66 (dd, J=14.7, 5.9 Hz, 1H), 1.54-1.45 (m, 1H), 1.36 (d, J=6.5 Hz, 3H), 1.03 (s, 3H), 0.97 (s, 3H). ¹⁹F NMR (282 MHz, CDCl₃) δ-78.83 (s, 3F). The above crude product (12.02 g) was dissolved in diethyl ether (20 mL) and diluted with heptanes (80 mL) and cooled in an ice-water bath. HCl in 1,4-dioxane (10.5 mL of 4 M, 42.000 mmol) was added dropwise. The mixture was stirred at rt for 30 min and filtered to give (2R)-5,5,5-trifluoro-4,4-dimethyl-2-[[(1R)-1-phenylethyl]amino]pentan-1-ol (hydrochloride salt) (11.56 g, 98%) as a white solid. ¹H NMR (300 MHz, DMSO-d₆) δ 9.57 (br. s., 1H), 9.25 (t, J=9.8 Hz, 1H), 7.80-7.59 (m, 2H), 7.53-7.32 (m, 3H), 5.63 (br. s., 1H), 4.58 (t, J=6.3 Hz, 1H), 3.81-3.65 (m, 1H), 3.64-3.51 (m, 1H), 2.91-2.74 (m, 1H), 1.98-1.85 (m, 1H), 1.85-1.74 (m, 1H), 1.63 (d, J=6.8 Hz, 3H), 0.91 (s, 3H), 0.88 (s, 3H). ¹⁹F NMR (282 MHz, DMSO-d₆) δ-77.71 (s, 3F).ESI-MS m/z calc. 289.16534, found 290.2 (M+1)⁺; Retention time: 2.08 minutes; LC method U.

### Step 6: (2R)-2-Amino-5,5,5-trifluoro-4,4-dimethyl-pentan-1-ol

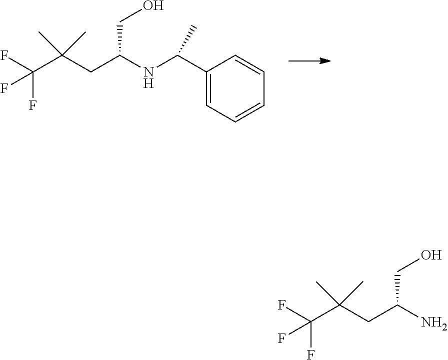 [SMILES: C[C@@H](N[C@@H](CO)CC(C)(C)C(F)(F)F)c1ccccc1 ; CC(C)(C[C@@H](N)CO)C(F)(F)F]

To a solution of (2R)-5,5,5-trifluoro-4,4-dimethyl-2-[[(1R)-1-phenylethyl]amino]pentan-1-ol (hydrochloride salt) (11.56 g, 35.482 mmol) in EtOH (200 mL) was added 10% palladium on carbon, 50% wet (5 g, 2.3492 mmol). The mixture was hydrogenated in a Parr shaker hydrogenation apparatus at 40 psi of hydrogen at rt for 9 h. More 10% palladium on carbon, 50% wet (1 g, 0.4698 mmol) was added. The mixture was shaken at 40 psi for 7 h. The mixture was filtered through Celite and washed with EtOH. The filtrate was concentrated. The residue (7.9 g) was triturated with a mixture of 2-methyltetrahydrofuran (28 mL) and heptanes (200 mL) and stirred overnight. The mixture was filtered, and the white solid was dried on vacuum to give (2R)-2-amino-5,5,5-trifluoro-4,4-dimethyl-pentan-1-ol (hydrochloride salt) (7.66 g, 93%) as a white solid. ¹H NMR (300 MHz, DMSO-d₆) δ 8.08 (br. s., 3H), 5.46 (t, J=5.0 Hz, 1H), 3.67-3.52 (m, 1H), 3.43 (dt, J=11.7, 5.8 Hz, 1H), 3.29-3.16 (m, 1H), 1.88-1.73 (m, 1H), 1.72-1.58 (m, 1H), 1.15 (s, 3H), 1.10 (s, 3H). ¹⁹F NMR (282 MHz, DMSO-d₆) δ-78.07 (s, 3F). ESI-MS m/z calc. 185.10275, found 186.2 (M+1)⁺; Retention time: 0.64 minutes; LC method U.

### Step 7: 3-[[4-[(2R)-2-Amino-5,5,5-trifluoro-4,4-dimethyl-pentoxy]-6-(2,6-dimethylphenyl)pyrimidin-2-yl]sulfamoyl]benzoic Acid

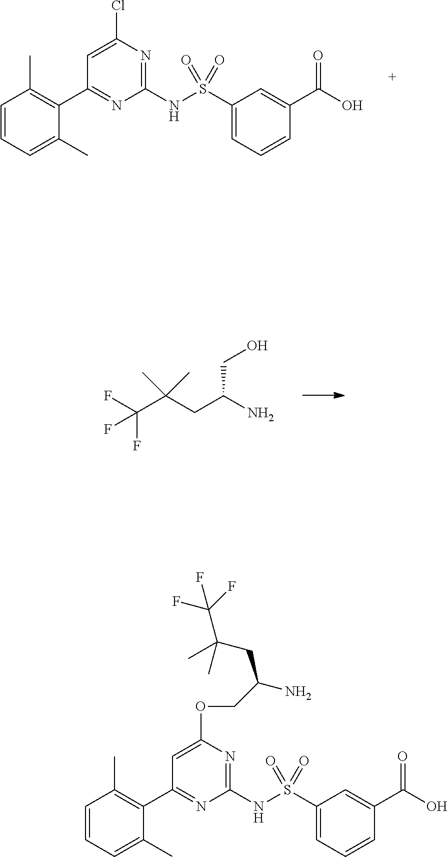 [SMILES: Cc1cccc(C)c1-c1cc(Cl)nc(NS(=O)(=O)c2cccc(C(=O)O)c2)n1 ; CC(C)(C[C@@H](N)CO)C(F)(F)F ; Cc1cccc(C)c1-c1cc(OC[C@H](N)CC(C)(C)C(F)(F)F)nc(NS(=O)(=O)c2cccc(C(=O)O)c2)n1]

3-[[4-Chloro-6-(2,6-dimethylphenyl)pyrimidin-2-yl]sulfamoyl]benzoic acid (6.12 g, 14.65 mmol) and (2R)-2-amino-5,5,5-trifluoro-4,4-dimethyl-pentan-1-ol (hydrochloride salt) (3.27 g, 14.75 mmol) were combined in THE (30 mL) and the resulting suspension was cooled in a water-ice bath. Sodium tert-butoxide (5.63 g, 58.58 mmol) was added inducing rapid partial dissolution of the solid. After 5 minutes, the cooling bath was removed, and the reaction was stirred at room temperature for 1 hour (90% conversion). More (2R)-2-amino-5,5,5-trifluoro-4,4-dimethyl-pentan-1-ol (hydrochloride salt) (363 mg, 1.638 mmol) was added and the mixture was stirred for one hour (no change). More sodium tert-butoxide (744 mg, 7.742 mmol) was added and the mixture was stirred for 40 min (96% conversion). Ethyl acetate (100 mL), HCl (90 mL of 1 M, 90.00 mmol) and brine (50 mL) were added and the resulting two phases were separated. The organic phase was washed with brine (50 mL), dried over sodium sulfate and concentrated. The residue was triturated in EtOAc/MeOH/Hexanes and the solvents were evaporated to give 3-[[4-[(2R)-2-amino-5,5,5-trifluoro-4,4-dimethyl-pentoxy]-6-(2,6-dimethylphenyl)pyrimidin-2-yl]sulfamoyl]benzoic acid (hydrochloride salt) (8.88 g, 93%) as a cream solid. ¹H NMR (400 MHz, DMSO-d₆) δ 13.15 (very broad s, 1H), 8.61-8.30 (m, 4H), 8.14 (dd, J 7.9, 1.9 Hz, 2H), 7.69 (t, J 7.8 Hz, 1H), 7.31-7.20 (m, 1H), 7.12 (d, J 7.6 Hz, 2H), 6.33 (s, 1H), 4.43 (dd, J=11.9, 3.3 Hz, 1H), 4.29-4.15 (m, 1H), 3.74 (s, 1H), 2.06-1.94 (broad m, 6H), 1.94-1.85 (m, 2H), 1.22 (s, 3H), 1.16 (s, 3H). ESI-MS m/z calc. 566.1811, found 567.62 (M+1)⁺; Retention time: 1.13 minutes (LC method A).

### Example 7: Preparation of 3-[[4-[(2R)-2-amino-3-[1-(trifluoromethyl)cyclopropyl]propoxy]-6-(2,6-dimethylphenyl)pyrimidin-2-yl]sulfamoyl]benzoic Acid

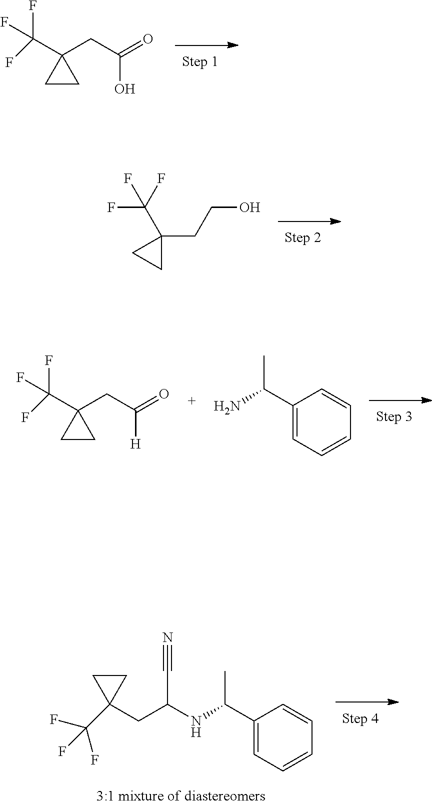

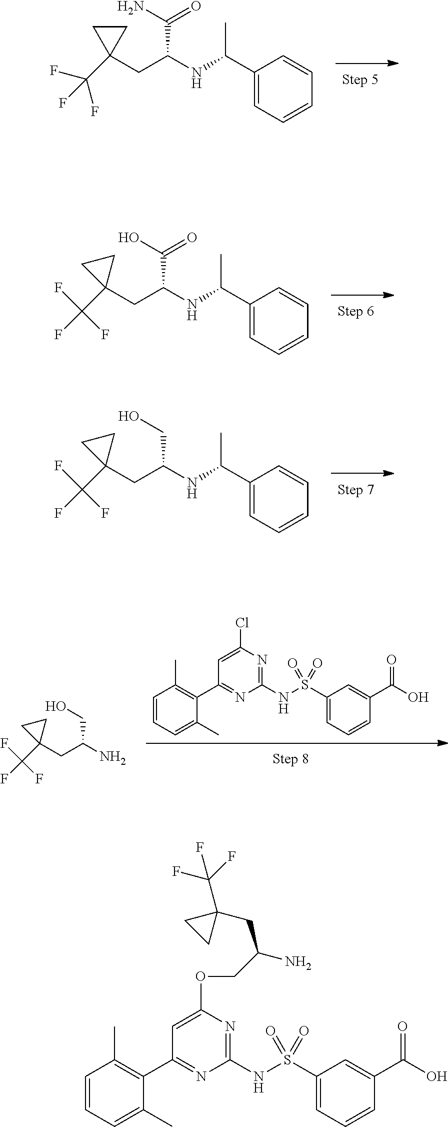 [SMILES: C[C@@H](N[C@H](CC1(C(F)(F)F)CC1)C(N)=O)c1ccccc1 ; C[C@@H](N[C@H](CC1(C(F)(F)F)CC1)C(=O)O)c1ccccc1 ; C[C@@H](N[C@@H](CO)CC1(C(F)(F)F)CC1)c1ccccc1 ; N[C@@H](CO)CC1(C(F)(F)F)CC1 ; Cc1cccc(C)c1-c1cc(Cl)nc(NS(=O)(=O)c2cccc(C(=O)O)c2)n1 ; Cc1cccc(C)c1-c1cc(OC[C@H](N)CC2(C(F)(F)F)CC2)nc(NS(=O)(=O)c2cccc(C(=O)O)c2)n1]

### Step 1: 2-[1-(Trifluoromethyl)cyclopropyl]ethanol

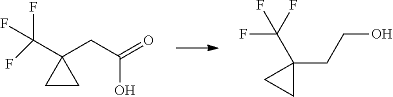 [SMILES: O=C(O)CC1(C(F)(F)F)CC1 ; OCCC1(C(F)(F)F)CC1]

LAH (49.868 g, 1.3139 mol) was added to THE (1700 mL) under nitrogen and the mixture was stirred for 30 minutes before being cooled to 0° C. 2-[1-(trifluoromethyl)cyclopropyl]acetic acid (190.91 g, 1.0107 mol) in THE (500 mL) was added dropwise while controlling the temperature <5° C. The mixture was allowed to warm up to room temperature and stirred for 24 hours. The resulting suspension was cooled to 0° C., water (50 mL) was added very slowly, followed by 15% w/w sodium hydroxide (50 mL) and water (150 mL). The mixture was stirred at 0° C. for 30 minutes, and filtered through Celite pad, the filter cake was washed with THE (2×500 mL). The combined filtrates were evaporated in vacuo to give 2-[1-(trifluoromethyl)cyclopropyl]ethanol (160.27 g, 98%) as amber oil containing ˜5% w/w of THE (by NMR). ¹H NMR (250 MHz, DMSO-d₆) δ 4.57 (t, J 5.2 Hz, 1H), 3.55-3.39 (m, 2H), 1.74 (t, J 7.3 Hz, 2H), 1.00-0.58 (m, 4H).

### Step 2: 2-[1-(Trifluoromethyl)cyclopropyl]acetaldehyde

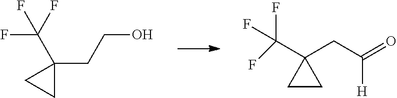 [SMILES: OCCC1(C(F)(F)F)CC1 ; O=CCC1(C(F)(F)F)CC1]

To a solution of 2-[1-(trifluoromethyl)cyclopropyl]ethanol (80 g, 467.1 mmol) in methylene chloride (1.1 L) was stirred at room temperature and treated with Dess-Martin periodinane (250 g, 589.4 mmol) portionwise (exothermic! cooled in ice bath and kept T<15° C.). To the mixture was added water (12 mL, 666.1 mmol) slowly added over 0.5 h (exothermic during addition up to 33° C., kept between 20 and 33° C. by cooling with cold water) giving a thick suspension. After the addition, the pale-yellow fine suspension was stirred at room temperature for 18 h. The yellow suspension was diluted with diethylether (500 mL) (yellow suspension) and stirred for 30 min. The slurry was filtered over Celite and the precipitate washed with 100 mL of Diethylether. diethylether. The organic phase was carefully treated with a saturated aqueous solution of sodium carbonate (500 ml, strong gas evolution, pH ˜10 at the end). The three-phase mixture was stirred at room temperature for 1 h and the solid was removed by filtration (large glass fritt). The phases (yellow cloudy Diethylether phase, colorless water phase) were separated and the organic phase was washed once more with a saturated aqueous solution of sodium carbonate (250 mL), once with 1M sodium thiosulfate (250 mL) and once with brine (250 mL). The aqueous phases were back extracted once with diethyl ether (150 mL) and the combined organic phases were dried, filtered and evaporated to give 2-[1-(trifluoromethyl)cyclopropyl]acetaldehyde (40 g, 56%) as a yellow liquid.

### Step 3: 2-[[(1R)-1-Phenylethyl]amino]-3-[1-(trifluoromethyl)cyclopropyl]propanenitrile

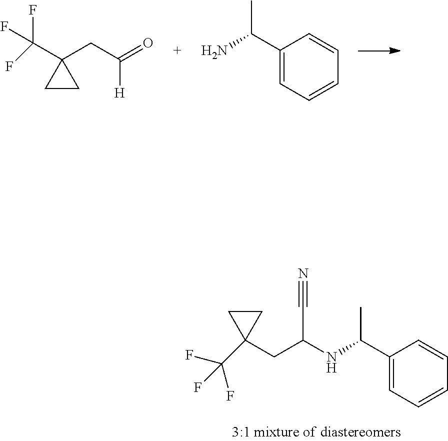

2-[1-(Trifluoromethyl)cyclopropyl]acetaldehyde (102 g, 670.5 mmol) in MeOH (700 mL) was treated with (1R)-1-phenylethanamine (86 mL, 667.1 mmol) and cooled in an ice bath. The solution was treated with acetic acid (38 mL, 668.2 mmol), stirred for 20 min in the ice bath, then solid NaCN (CAUTION, 33 g, 673.4 mmol) was added in one portion and the suspension was stirred in the melting ice bath for 14 hours. The solution was concentrated under reduced pressure (CAUTION, HCN!, the exhaust from the pump was running through a bleach trap) and the residue was extracted with MTBE (1000 mL) and saturated sodium carbonate/water 1:1 (1000 mL) and washed with brine (350 mL). The aqueous phases were back extracted once with MTBE (250 mL) and the combined organic phases were dried, filtered and evaporated to give 2-[[(1R)-1-phenylethyl]amino]-3-[1-(trifluoromethyl)cyclopropyl]propanenitrile (180.8 g, 96%) as 3:1 mixture of diastereomers. ESI-MS m/z calc. 282.13437, found 283.0 (M+1)⁺; Retention time: 1.69 minutes (major isomer) and 1.62 minutes (minor isomer), LC method A.

### Step 4: (2R)-2-[[(1R)-1-Phenylethyl]amino]-3-[1-(trifluoromethyl)cyclopropyl]propenamide

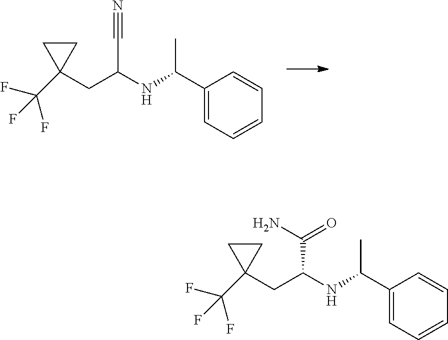 [SMILES: C[C@@H](NC(C#N)CC1(C(F)(F)F)CC1)c1ccccc1 ; C[C@@H](N[C@H](CC1(C(F)(F)F)CC1)C(N)=O)c1ccccc1]

In a 2 L flask equipped with mechanical stirring and a temperature probe, sulfuric acid (285 mL of 18 M, 5.130 mol) was added it was cooled in an ice bath. At an internal temperature of 5° C., a solution of 2-[[(1R)-1-phenylethyl]amino]-3-[1-(trifluoromethyl)cyclopropyl]propanenitrile (180.8 g, 640.4 mmol, 3:1 mixture of diastereomers) in DCM (900 mL) was added dropwise over 20 minutes. The ice bath was removed, and the deep orange emulsion was stirred at room temperature for 18 h and at 30-40° C. for 2 h. The deep orange emulsion was carefully added to a mixture of ice and water (2.2 L) under mechanical stirring to give a yellow three phase mixture which was basified by slow addition of ammonium hydroxide (1.33 L of 30% w/w, 10.25 mol) under ice cooling (very exothermic, internal temperature kept between 10 and 25° C. by adding ice). The yellow emulsion was stirred for 10 minutes at room temperature (pH ˜10), diluted with DCM (500 mL) and the phases were separated. The aqueous phase was washed twice more with DCM (400 and 200 mL) and the combined organic phases were washed once with water/brine 1:1 (500 mL). The DCM phase was dried, filtered and evaporated to give crude 2-[[(1R)-1-phenylethyl]amino]-3-[1-(trifluoromethyl)cyclopropyl]propanamide (189.5 g, 99%) as a yellow-orange oil. ESI-MS m z calc. 300.14496, found 301.0 (M+1)⁺; Retention time: 1.40 minutes (major isomer) and 1.50 minutes (minor isomer) (3:1 mixture of diastereomers). The product was dissolved in ethanol (1.5 L) and it was treated quickly with HCl (240 mL of 4 M, 960.0 mmol) (4M in dioxane) and the resulting thick suspension was stirred at room temperature overnight under mechanic stirring. The solid was collected by filtration, washed with cold ethanol and dried under vacuum with a nitrogen bleed at 40-45° C. to give (2R)-2-[[(1R)-1-phenylethyl]amino]-3-[1-(trifluoromethyl)cyclopropyl]propanamide (hydrochloride salt) (147 g, 68%). ¹H NMR (499 MHz, DMSO-d₆) δ 9.74 (d, J=67.9 Hz, 2H), 8.16-7.94 (m, 1H), 7.86 (s, 1H), 7.64-7.51 (m, 2H), 7.51-7.34 (m, 3H), 4.22 (s, 1H), 3.46-3.37 (m, 1H), 2.45 (d, J=15.9 Hz, 1H), 1.85 (dd, J 15.1, 10.4 Hz, 1H), 1.58 (d, J 6.7 Hz, 3H), 0.89 (pd, J 9.6, 9.2, 4.3 Hz, 2H), 0.84-0.66 (m, 2H). ESI-MS m/z calc. 300.14496, found 301.0 (M+1)⁺; Retention time: 1.40 minutes (major isomer) and 1.40 minutes (minor isomer), 97:3 mixture of diastereomers (LC method V).

### Step 5: (2R)-2-[[(1R)-1-Phenylethyl]amino]-3-[1-(trifluoromethyl)cyclopropyl]propanoic Acid

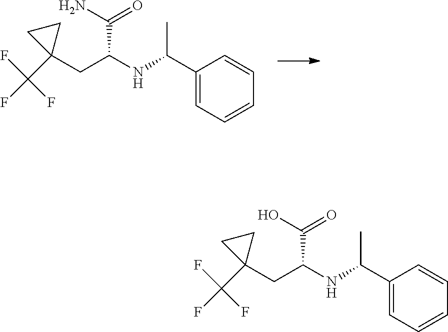 [SMILES: C[C@@H](N[C@H](CC1(C(F)(F)F)CC1)C(N)=O)c1ccccc1 ; C[C@@H](N[C@H](CC1(C(F)(F)F)CC1)C(=O)O)c1ccccc1]

In a 5 L flask equipped with mechanical stirring, (2R)-2-[[(1R)-1-phenylethyl]amino]-3-[1-(trifluoromethyl)cyclopropyl]propanamide (hydrochloride salt) (147 g, 436.5 mmol) was added to acetic acid (735 mL) under stirring and the thick colorless suspension was treated with HCl (1.3 L of 12 M, 15.60 mol). The colorless suspension was carefully heated to 60-65° C. (strong foaming, acetic acid (145 mL) was added) and the suspension was stirred at 60-65° C. for 16 h. The suspension was then slowly heated to 100° C. (over 4 h, strong foaming) and the resulting solution was stirred at 100° C. for another 20 h. The pale-yellow solution was concentrated under reduced pressure at 65° C. to a semisolid mass and it was treated with water (1.5 L). The thick suspension was heated to 70-80° C. and left to cool to room temperature under stirring for 2 h. The solid was collected by filtration, washed with water and sucked dry overnight. The wet solid was further dried under reduced pressure at 50-60° C. for 4 h to give (2R)-2-[[(1R)-1-phenylethyl]amino]-3-[1-(trifluoromethyl)cyclopropyl]propanoic acid (hydrochloride salt) (135 g, 92%) as an off-white solid. ESI-MS m/z calc. 301.12897, found 302.0 (M+1)⁺; Retention time: 1.82 minutes; (LC method V).

### Step 6: (2R)-2-[[(1R)-1-phenylethyl]amino]-3-[1-(trifluoromethyl)cyclopropyl]propan-1-ol

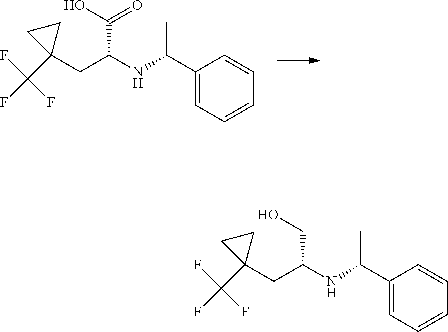 [SMILES: C[C@@H](N[C@H](CC1(C(F)(F)F)CC1)C(=O)O)c1ccccc1 ; C[C@@H](N[C@@H](CO)CC1(C(F)(F)F)CC1)c1ccccc1]

In a 5 L flask equipped with mechanical stirring and under dry nitrogen atmosphere, (2R)-2-[[(1R)-1-phenylethyl]amino]-3-[1-(trifluoromethyl)cyclopropyl]propanoic acid (hydrochloride salt) (135 g, 399.7 mmol) was suspended in THE (2 L) (thick suspension). It was heated to 35-40° C. and LAH (47.3 g, 1.214 mol) (pellets) was slowly added over 1 hour, while keeping the internal temperature between 30 and 40° C. by external cooling. The mixture was stirred for 1 hour at 30-40° C. (almost no hydrogen evolution anymore, grey suspension, most starting material in solution) and it was heated at 50-55° C. for 1 h. The grey suspension was left stirring in the cooling heating mantel overnight. The grey suspension was cooled in an ice bath and quenched by careful addition of water (44 mL, 2.442 mol), NaOH (41 mL of 6 M, 246.0 mmol) and water (44 mL, 2.442 mol) (high exotherm with first water addition, kept between 5° C. and 30° C. by cooling). The grey suspension was heated to 50-55° C. for 1 h, by which time a colorless suspension was obtained. The warm suspension was filtered over a pad of Celite covered over magnesium sulfate. The solids were washed with hot THF and evaporated to give crude (2R)-2-[[(1R)-1-phenylethyl]amino]-3-[1-(trifluoromethyl)cyclopropyl]propan-1-ol (121 g, 105%) as an oil. The crude was dissolved in diethyl ether (1 L, clear solution) and slowly treated with HCl (101 mL of 4 M, 404.0 mmol) (4M in dioxane) under cooling. The resulting thick suspension was stirred at room temperature for 1 h, the solid collected by filtration, washed with diethyl ether and dried under reduced pressure at 40-45° C. with a nitrogen bleed to give (2R)-2-[[(1R)-1-phenylethyl]amino]-3-[1-(trifluoromethyl)cyclopropyl]propan-1-ol (hydrochloride salt) (126.6 g, 98%) as an off-white solid. ¹H NMR (500 MHz, DMSO-d₆) δ 9.34 (s, 2H), 7.66 (d, J=7.4 Hz, 2H), 7.43 (dt, J=25.1, 7.4 Hz, 3H), 5.59 (s, 1H), 4.58 (q, J=6.6 Hz, 1H), 3.83 (d, J 12.6 Hz, 1H), 3.62-3.54 (m, 1H), 2.89 (s, 1H), 2.33-2.24 (m, 1H), 1.67-1.51 (m, 4H), 0.97-0.81 (m, 3H), 0.71 (s, 1H). ESI-MS m/z calc. 287.1497, found 288.0 (M+1)⁺; Retention time: 0.99 minutes (LC method A).

### Step 7: (2R)-2-Amino-3-[1-(trifluoromethyl)cyclopropyl]propan-1-ol

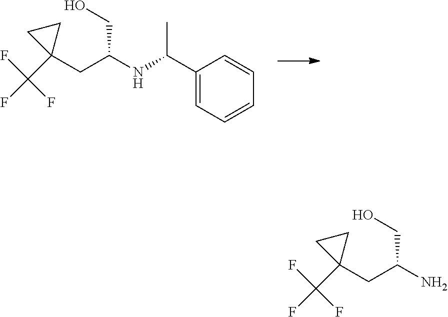 [SMILES: C[C@@H](N[C@@H](CO)CC1(C(F)(F)F)CC1)c1ccccc1 ; N[C@@H](CO)CC1(C(F)(F)F)CC1]

In a 1 L hydrogenation reactor, (2R)-2-[[(1R)-1-phenylethyl]amino]-3-[1-(trifluoromethyl)cyclopropyl]propan-1-ol (hydrochloride salt) (63.3 g, 195.5 mmol) was dissolved in EtOH (630 mL) (under warming), and it was treated with Pd/C (6.3 g of 10% w/w, 5.920 mmol) (12.5 g of 50% water wet) and the reaction was stirred under 2 bar of hydrogen at 40° C. for 24 h. The reaction mixture was filtered over Celite. The pad was washed with ethanol and the colorless filtrate was evaporated to a solid mass, which was triturated with diethyl ether. The suspension was stirred at room temperature for 1 h. The solid was filtered, washed with plenty of diethyl ether and dried to give (2R)-2-amino-3-[1-(trifluoromethyl)cyclopropyl]propan-1-ol (hydrochloride salt) (41.8 g, 97%) as an off-white solid. ¹H NMR (500 MHz, DMSO-d₆) δ 8.18 (s, 3H), 5.45 (t, J 4.9 Hz, 1H), 3.71 (dt, J 11.6, 3.9 Hz, 1H), 3.55 (dt, J 11.2, 5.4 Hz, 1H), 3.24 (h, J 4.7 Hz, 1H), 2.08 (dd, J 15.1, 5.4 Hz, 1H), 1.69 (dd, J 15.1, 9.4 Hz, 1H), 0.97 (h, J 6.5, 5.9 Hz, 2H), 0.86 (s, 2H). ESI-MS m/z calc. 183.0871, found 184.0 (M+1)⁺; Retention time: 0.65 minutes; LC method A.

### Step 8: 3-[[4-[(2R)-2-Amino-3-[1-(trifluoromethyl)cyclopropyl]propoxy]-6-(2,6-dimethylphenyl)pyrimidin-2-yl]sulfamoyl]benzoic Acid

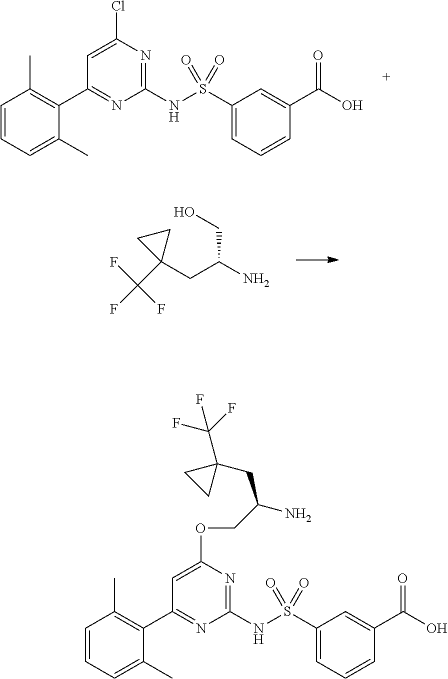 [SMILES: Cc1cccc(C)c1-c1cc(Cl)nc(NS(=O)(=O)c2cccc(C(=O)O)c2)n1 ; N[C@@H](CO)CC1(C(F)(F)F)CC1 ; Cc1cccc(C)c1-c1cc(OC[C@H](N)CC2(C(F)(F)F)CC2)nc(NS(=O)(=O)c2cccc(C(=O)O)c2)n1]

3-[[4-Chloro-6-(2,6-dimethylphenyl)pyrimidin-2-yl]sulfamoyl]benzoic acid (19.09 g, 45.68 mmol) and (2R)-2-amino-3-[1-(trifluoromethyl)cyclopropyl]propan-1-ol (hydrochloride salt) (10.18 g, 46.35 mmol) were dissolved in THE (100 mL) and cooled in an ice water bath. Sodium tert-butoxide (18.14 g, 188.8 mmol) was added and the reaction was allowed to warm to room temperature. The reaction was stirred for 1 h, then partitioned between ethyl acetate (500 mL) and aqueous HCl (275 mL of 1 M, 275.0 mmol). The organics were separated, washed with brine, dried over sodium sulfate and evaporated to give 3-[[4-[(2R)-2-amino-3-[1-(trifluoromethyl)cyclopropyl]propoxy]-6-(2,6-dimethylphenyl)pyrimidin-2-yl]sulfamoyl]benzoic acid (hydrochloride salt) (26.74 g, 94%). ESI-MS m/z calc. 564.1654, found 565.1 (M+1)⁺; Retention time: 0.48 minutes; LC method D.

### Example 8: Preparation of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one

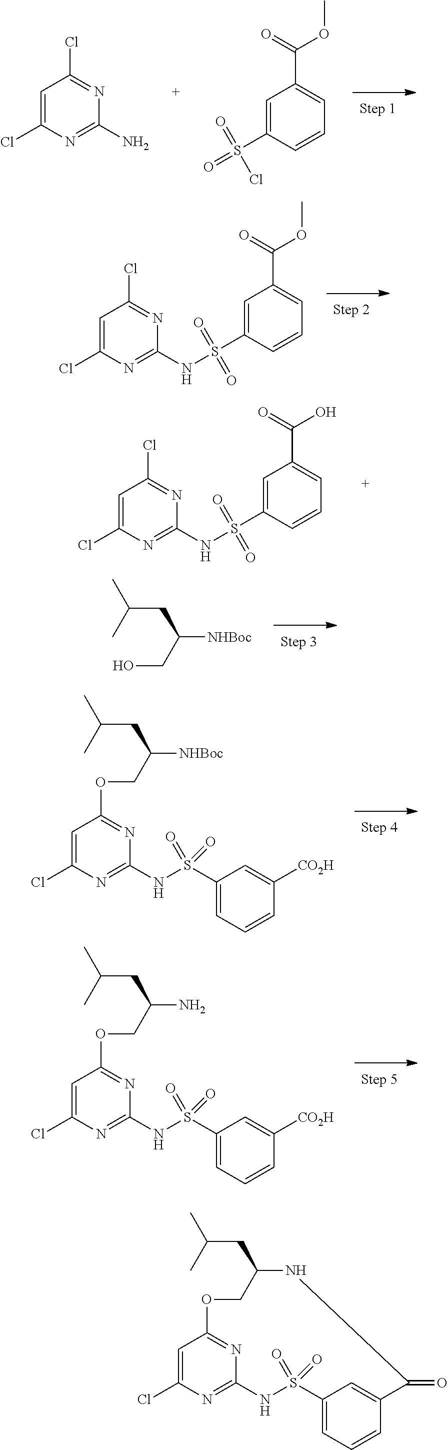

### Step 1: Methyl 3-[(4,6-dichloropyrimidin-2-yl)sulfamoyl]benzoate

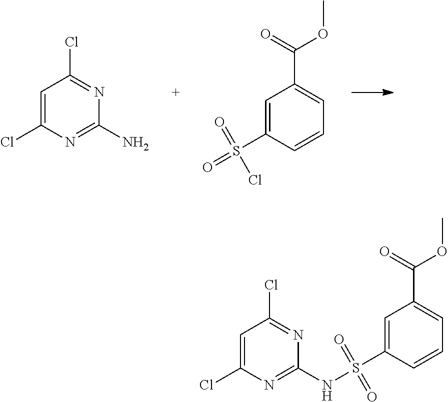 [SMILES: Nc1nc(Cl)cc(Cl)n1 ; COC(=O)c1cccc(S(=O)(=O)Cl)c1 ; COC(=O)c1cccc(S(=O)(=O)Nc2nc(Cl)cc(Cl)n2)c1]

NaH (18.22 g, 455.54 mmol) was suspended in NMP (300 mL). A solution of 4,6-dichloropyrimidin-2-amine (52.9 g, 316.12 mmol) in NMP (200 mL) was added at 0° C. The bath was removed, and the reaction mixture was allowed to stir at room temperature for 60 minutes. After cooling again to 0° C., a solution of methyl 3-chlorosulfonylbenzoate (90.9 g, 379.63 mmol) in NMP (200 mL) was slowly added. The reaction mixture was allowed to stir at 0° C. for 2 hour and it was quenched with the slow and careful addition of aqueous 3 M HCl (400 mL, strong gas evolution). After the gas evolution stopped, the mixture was added to aqueous 3M HCl (750 mL). The resulting white solid was extracted with EtOAc (2×750 mL). The organic layers were combined, washed with brine (2×750 mL), dried over sodium sulfate, filtered and concentrated under reduced pressure. The resulting oil was purified by chromatography on a 1 kg silica gel column eluting with a 0-40% EtOAc/hexane gradient over 1 day to give crude methyl 3-[(4,6-dichloropyrimidin-2-yl)sulfamoyl]benzoate. It was triturated with 50% hexanes/EtOAc (500 mL) to give a first batch of material (white solid, 66.33 g). The filtrate was concentrated and triturated with 50% hexanes/EtOAc (50 mL) to give a second batch (white solid, 8.1 g). The filtrate was concentrated to give more material as a brown solid (7.67 g). The total amount of methyl 3-[(4,6-dichloropyrimidin-2-yl)sulfamoyl]benzoate was 82.1 g (61% yield). ESI-MS m/z calc. 360.9691, found 362.0 (M+1)⁺; Retention time: 5.12 minutes; LC method S.

### Step 2: 3-[(4,6-Dichloropyrimidin-2-yl)sulfamoyl]benzoic Acid

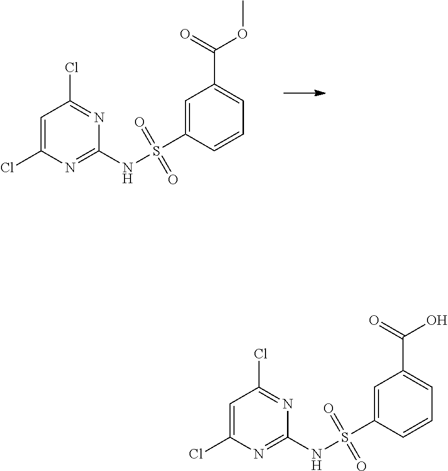 [SMILES: COC(=O)c1cccc(S(=O)(=O)Nc2nc(Cl)cc(Cl)n2)c1 ; O=C(O)c1cccc(S(=O)(=O)Nc2nc(Cl)cc(Cl)n2)c1]

Methyl 3-[(4,6-dichloropyrimidin-2-yl)sulfamoyl]benzoate (66.33 g, 173.98 mmol) was dissolved in THE (330 mL) and iPrOH (66 mL). The solution was cooled to 0° C. Aqueous cold NaOH (732 mL of 1 M, 732.00 mmol) was added in one portion. The reaction mixture was allowed to stir at 0° C. for 1.5 hours. The mixture was extracted with DCM (2×500 mL) and the aqueous layer was acidified with 3M HCl (500 mL). The mixture was filtered to provide a white solid which was dried under vacuum over the weekend to afford 3-[(4,6-dichloropyrimidin-2-yl)sulfamoyl]benzoic acid (58.67 g, 96%).ESI-MS m/z calc. 346.95343, found 348.0 (M+2)⁺; Retention time: 2.42 minutes; LC method T.

### Step 3: 3-[[4-[(2R)-2-(tert-Butoxycarbonylamino)-4-methyl-pentoxy]-6-chloro-pyrimidin-2-yl]sulfamoyl]benzoic Acid

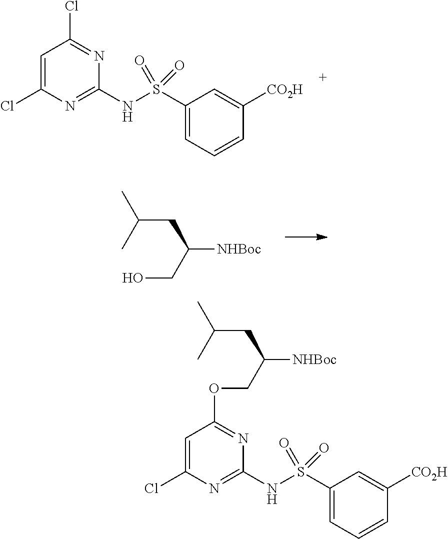

3-[(4,6-Dichloropyrimidin-2-yl)sulfamoyl]benzoic acid (56.11 g, 159.55 mmol) and tert-butyl N-[(1R)-1-(hydroxymethyl)-3-methyl-butyl]carbamate (42 g, 187.48 mmol) in THE (800 mL) was stirred at 30° C. . Sodium tert-butoxide (48 g, 484.48 mmol) was added. The reaction mixture was stirred at 30° C. for 3 hours. The reaction mixture was diluted with water (500 mL) and washed with DCM (2×1 L). The aqueous layer (combined with another reaction run on 3.2 g scale) was acidified with 5% HCl and extracted with EtOAc (1 L). The organic layer was washed with brine (3×500 L), dried with sodium sulfate and evaporated to provide 3-[[4-[(2R)-2-(tert-butoxycarbonylamino)-4-methyl-pentoxy]-6-chloro-pyrimidin-2-yl]sulfamoyl]benzoic acid (95 g, 56% combined yield) as an off-white solid. ESI-MS m/z calc. 528.14453, found 529.0 (M+1)⁺; Retention time: 6.24 minutes; LC method S.

### Step 4: 3-[[4-[(2R)-2-Amino-4-methyl-pentoxy]-6-chloro-pyrimidin-2-yl]sulfamoyl]benzoic Acid

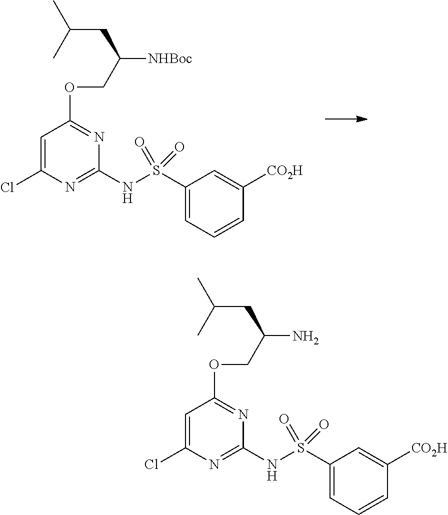

3-[[4-[(2R)-2-(tert-Butoxycarbonylamino)-4-methyl-pentoxy]-6-chloro-pyrimidin-2-yl]sulfamoyl]benzoic acid (95 g, 89.791 mmol) was dissolved in a solution of HCl in dioxane (378 mL of 4 M, 1.5120 mol). After stirring at room temperature for 0.5 h, the volatiles were removed under reduced pressure to provide 3-[[4-[(2R)-2-amino-4-methyl-pentoxy]-6-chloro-pyrimidin-2-yl]sulfamoyl]benzoic acid (80 g, 104%, 50% pure) as an off-white foam which was used without further purification. ESI-MS m/z calc. 428.09213, found 429.0 (M+1)⁺; Retention time: 4.13 minutes; LC method S.

### Step 5: (11R)-6-Chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one

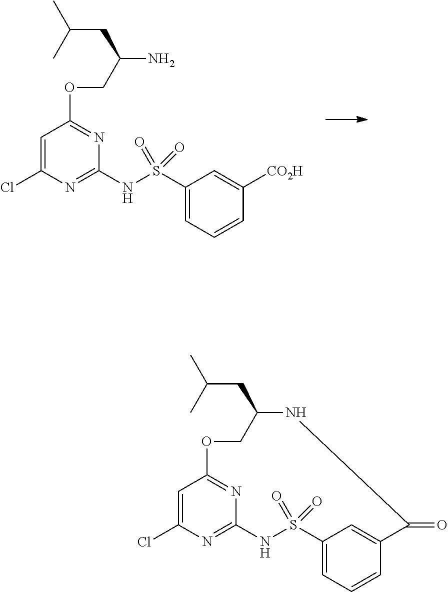 [SMILES: CC(C)C[C@@H](N)COc1cc(Cl)nc(NS(=O)(=O)c2cccc(C(=O)O)c2)n1 ; CC(C)C[C@@H]1COc2cc(Cl)nc(n2)NS(=O)(=O)c2cccc(c2)C(=O)N1]

The reaction was carried out in several batches, based on 8×10 g and 1×5 g of starting material. For one 10 g batch reaction, 3-[[4-[(2R)-2-Amino-4-methyl-pentoxy]-6-chloro-pyrimidin-2-yl]sulfamoyl]benzoic acid (10 g, 11.658 mmol) was dissolved in DMF (400 mL) and the solution was cooled down to −5° C. (ice-salt bath). Triethylamine (1.1797 g, 1.6249 mL, 11.542 mmol) was added, followed by HATU (4.4327 g, 11.308 mmol). After stirring for 10 minutes, the solution was diluted with EtOAc (600 mL) and washed with aqueous HCl (1 M, 2×400 mL) and brine (3×400 mL). The organic layer was dried over sodium sulfate, filtered and concentrated under reduced pressure to give a crude material which was combined with all other batches. The overall crude solid was suspended in EtOAc (200 mL) at 65° C. and the suspension was cooled down to rt. A solid was collected by vacuum filtration to provide material which was stirred in water (500 mL) overnight. The EtOAc (200 mL) filtrate was concentrated to obtain more solid which was suspended in EtOAc (50 mL) at 65° C. and cooled down to rt. The solid was collected by filtration, and it was stirred in water (100 mL) and filtered to get another amount of solid. A total amount of 12.75 g of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one was isolated as a white solid (overall yield 30%). ¹H NMR (500 MHz, DMSO-d₆) δ 12.32 (s, 1H), 8.47 (s, 1H), 7.98 (dt, J 6.8, 2.0 Hz, 1H), 7.91 (d, J 9.9 Hz, 1H), 7.80-7.69 (m, 2H), 6.76 (s, 1H), 5.07 (dd, J 11.1, 3.9 Hz, 1H), 3.81 (t, J 11.2 Hz, 1H), 3.12 (q, J 10.5 Hz, 1H), 1.60-1.41 (m, 2H), 1.23-1.10 (m, 1H), 0.77 (d, J 6.6 Hz, 3H), 0.26 (d, J 6.5 Hz, 3H). ESI-MS m/z calc. 410.08154, found 411.0 (M+1)⁺; Retention time: 2.08 minutes; LC method T.

### Example 9: Preparation of 11R)-6-chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one

### Step 1: (11R)-6-Chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one

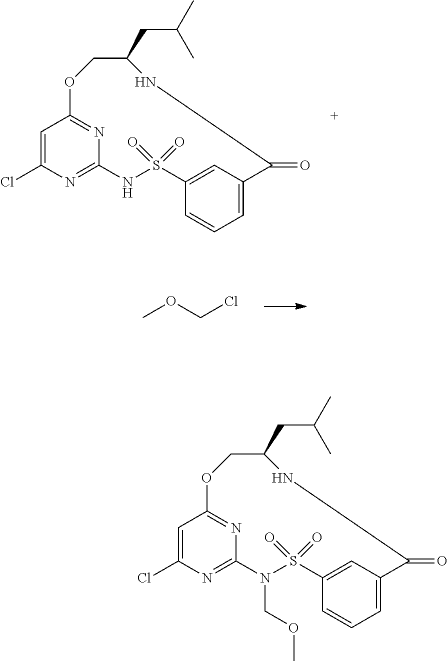

(11R)-6-Chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (1 g, 2.434 mmol) was dissolved in acetonitrile (15.0 mL): DCE (15.0 mL). The mixture was treated with powdered Potassium carbonate (510 mg, 3.690 mmol) and chloro(methoxy)methane (215 μL, 2.831 mmol). The mixture was stirred at 20° C. for 16 h. The reaction mixture was filtered and concentrated. The crude product was purified by silica gel chromatography using a 40 g column eluting with 100% hexanes to 90% ethyl acetate in hexanes to afford a white solid (11R)-6-chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one (860 mg, 78%) ESI-MS m/z calc. 454.10776, found 455.2 (M+1)⁺; Retention time: 1.69 minutes (LC method A). ¹H NMR (500 MHz, DMSO-d₆) δ 8.58 (s, 1H), 8.10 (d, J 7.8 Hz, 1H), 7.95 (d, J 9.7 Hz, 1H), 7.88-7.71 (m, 2H), 6.95 (s, 1H), 5.61 (d, J 11.0 Hz, 1H), 5.53 (d, J 11.0 Hz, 1H), 5.06 (dd, J=11.3, 4.0 Hz, 1H), 3.87 (t, J=11.3 Hz, 1H), 3.22 (d, J=13.6 Hz, 1H), 3.10 (s, 3H), 1.60-1.42 (m, 2H), 1.20 (dd, J 13.4, 10.6 Hz, 1H), 0.78 (d, J 6.6 Hz, 3H), 0.28 (d, J 6.4 Hz, 3H).

### Example 10: Preparation of of 3-[1-(trifluoromethyl)cyclopropyl]propan-1-ol

### Step 1: 2-[1-(Trifluoromethyl)cyclopropyl]ethyl methanesulfonate

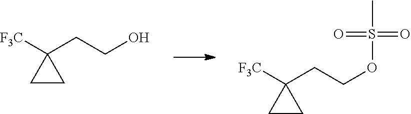

A 1000 mL, 3-neck round bottom flask was fitted with a mechanical stirrer, a cooling bath, a J-Kem temperature probe, an addition funnel and a nitrogen inlet/outlet. The vessel was charged under a nitrogen atmosphere with 2-[1-(trifluoromethyl)cyclopropyl]ethanol (125 g, 811.0 mmol) and 2-methyltetrahydrofuran (625 mL) which provided a clear colorless solution. Stirring was commenced and the pot temperature was recorded at 19° C. The vessel was then charged with triethylamine (124.3 mL, 891.8 mmol) added neat in one portion. The cooling bath was then charged with crushed ice/water and the pot temperature was lowered to 0° C. The addition funnel was charged with a solution of methanesulfonyl chloride (62.77 mL, 811.0 mmol) in 2-methyltetrahydrofuran (125 mL, 2 mL/g) which was subsequently added dropwise over 90 min which resulted in a white suspension and an exotherm to 1° C. The mixture was allowed to slowly warm to room temperature and continue to stir at room temperature for 1 h at which point the mixture was poured into ice cold water (250 mL) and then transferred to a separatory funnel. The organic was removed and washed with 20 wt % potassium bicarbonate solution (250 mL), dried over sodium sulfate (200 g) and then filtered through a glass frit Buchner funnel. The clear filtrate was concentrated under reduced pressure to provide 2-[1-(trifluoromethyl)cyclopropyl]ethyl methanesulfonate (185 g, 98%) as a clear pale yellow oil. ¹H NMR (400 MHz, Chloroform-d) δ 4.36 (ddt, J=7.1, 6.4, 0.7 Hz, 2H), 3.02 (s, 3H), 2.03 (t, J=7.1 Hz, 2H), 1.11-0.98 (m, 2H), 0.81-0.66 (m, 2H).

### Step 2: 3-[1-(Trifluoromethyl)cyclopropyl]propanenitrile

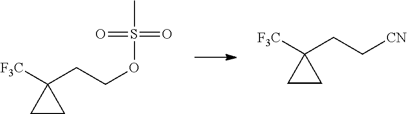

A 1000 mL, 3-neck round bottom flask was fitted with a mechanical stirrer, a heating mantle, a J-Kem temperature probe/controller, a water cooled reflux condenser and a nitrogen inlet/outlet. The vessel was charged under a nitrogen atmosphere with 2-[1-(trifluoromethyl)cyclopropyl]ethyl methanesulfonate (50 g, 215.3 mmol) and dimethyl sulfoxide (250 mL) which provided a clear pale yellow solution. Stirring was commenced and the pot temperature was recorded at 19° C. The vessel was charged with sodium cyanide (13.19 g, 269.1 mmol), added as a solid in one portion. The mixture was heated to a pot temperature of 70° C. and the condition was maintained for 24 h. Upon heating all of the sodium cyanide dissolved and the reaction mixture turned to a light amber suspension. After cooling to room temperature, the reaction mixture was poured into water (500 mL) and then transferred to a separatory funnel and partitioned with methyl tert-butyl ether (500 mL). The organic was removed and the residual aqueous was extracted with methyl tert-butyl ether (3×250 mL). The combined organic layers were washed with water (2×250 mL), dried over sodium sulfate (200 g) and then filtered through a glass frit Buchner funnel. The clear filtrate was concentrated under reduced pressure to provide 3-[1-(trifluoromethyl)cyclopropyl]propanenitrile (30 g, 85%) as a clear amber oil. ¹H NMR (400 MHz, Chloroform-d) δ 2.55 (t, J=7.6 Hz, 2H), 1.93 (t, J=7.7 Hz, 2H), 1.11-1.04 (m, 2H), 0.78-0.70 (m, 2H).

### Step 3: 3-[1-(Trifluoromethyl)cyclopropyl]propanoic Acid

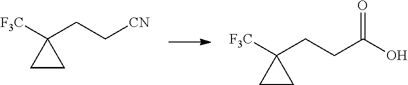 [SMILES: N#CCCC1(C(F)(F)F)CC1 ; O=C(O)CCC1(C(F)(F)F)CC1]

A 1000 mL, 3-neck round bottom flask was fitted with a mechanical stirrer, a heating mantle, a J-Kem temperature probe/controller, a water cooled reflux condenser and a nitrogen inlet/outlet. The vessel was subsequently charged under a nitrogen atmosphere with 3-[1-(trifluoromethyl)cyclopropyl]propanenitrile (25 g, 153.2 mmol) and ethyl alcohol (375 mL) which provided a clear amber solution. Stirring was commenced and the pot temperature was recorded at 19° C. The vessel was then charged with sodium hydroxide (102.1 mL of 6 M, 612.6 mmol), added in one portion. The resulting clear amber solution was heated to a pot temperature of 70° C. and the condition was maintained for 24 h. After cooling to room temperature, the reaction mixture was concentrated to remove the ethyl alcohol. The residual aqueous was diluted with water (150 mL) and then transferred to a separatory funnel and partitioned with methyl tert-butyl ether (50 mL). The aqueous was removed and the pH was adjusted to pH ˜ 1 with 6 M hydrochloric acid solution. The resulting aqueous solution was transferred to a separatory funnel and partitioned with methyl tert-butyl ether (250 mL). The organic was removed and the residual aqueous was extracted with methyl tert-butyl ether (2×150 mL). The combined organic was dried over sodium sulfate (150 g) and then filtered through a glass frit Buchner funnel. The clear filtrate was concentrated under reduced pressure to provide 3-[1-(trifluoromethyl)cyclopropyl]propanoic acid (26 g, 93%) as a clear amber oil. ¹H NMR (400 MHz, Chloroform-d) δ 2.63-2.50 (m, 2H), 1.96-1.84 (m, 2H), 1.03-0.95 (m, 2H), 0.66-0.58 (m, J=1.7 Hz, 2H).

### Step 4: 3-[1-(Trifluoromethyl)cyclopropyl]propan-1-ol

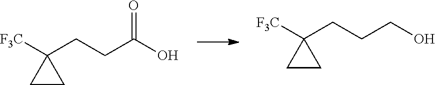 [SMILES: O=C(O)CCC1(C(F)(F)F)CC1 ; OCCCC1(C(F)(F)F)CC1]

A 1000 mL, 3-neck round bottom flask was fitted with a mechanical stirrer, a cooling bath, an addition funnel, a J-Kem temperature probe and a nitrogen inlet/outlet. The vessel was charged under a nitrogen atmosphere with lithium aluminum hydride pellets (6.775 g, 178.5 mmol). The vessel was then charged under a nitrogen atmosphere with tetrahydrofuran (250 mL). Stirring was commenced and the pot temperature was recorded at 20° C. The mixture was allowed to stir at room temperature for 0.5 h to allow the pellets to dissolve. The pot temperature of the resulting grey suspension was recorded at 24° C. The cooling bath was then charged with crushed ice/water and the pot temperature was lowered to 0° C. The addition funnel was charged with a solution of 3-[1-(trifluoromethyl)cyclopropyl]propanoic acid (25 g, 137.3 mmol) in tetrahydrofuran (75 mL, 3 mL/g) and the clear pale yellow solution was added dropwise over 1 h. After the addition was completed, the pot temperature of the resulting greyish-brown suspension was recorded at 5° C. The mixture was allowed to slowly warm to room temperature and continue to stir at room temperature for 24 h. The suspension was cooled to 0° C. with a crushed ice/water cooling bath and then quenched by the very slow dropwise addition of water (6.775 mL), followed by 15 wt % sodium hydroxide solution (6.775 mL) and then finally with water (20.32 mL). The pot temperature of the resulting white suspension was recorded at 5° C. The suspension was continued to stir at ˜5° C. for 30 min and then filtered through a glass frit Buchner funnel with a 20 mm layer of celite. The filter cake was displacement washed with tetrahydrofuran (2×150 mL) and then dried under vacuum for 15 min. The filtrate was dried over sodium sulfate (250 g) and then filtered through a glass frit Buchner funnel. The filtrate was concentrated under reduced pressure to provide a clear pale amber oil as the desired product, 3-[1-(trifluoromethyl)cyclopropyl]propan-1-ol (21.2 g, 92%). ¹H NMR (400 MHz, Chloroform-d) δ 3.65 (t, J=6.0 Hz, 2H), 1.78-1.59 (m, 4H), 0.99-0.91 (m, 2H), 0.59 (dp, J=4.7, 1.7 Hz, 2H).

### Example 11: Preparation of 6-[[4-Chloro-6-(2,6-dimethylphenyl)pyrimidin-2-yl]sulfamoyl]pyridine-2-carboxylic Acid

### Step 1: Methyl 6-benzylsulfanylpyridine-2-carboxylate

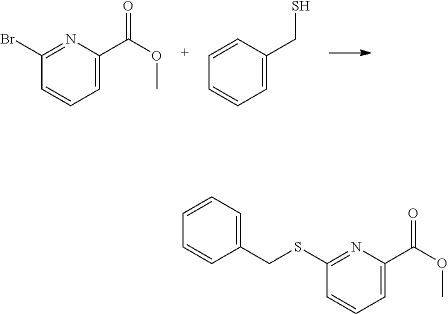 [SMILES: COC(=O)c1cccc(Br)n1 ; SCc1ccccc1 ; COC(=O)c1cccc(SCc2ccccc2)n1]

To a solution of phenylmethanethiol (28.408 g, 26.800 mL, 228.72 mmol) in THE (600 mL) was added NaH (11.200 g, 60% w/w, 280.03 mmol) in a few portions at 0° C. The slurry was warmed to room temperature and stirred for 30 min, then methyl 6-bromopyridine-2-carboxylate (50 g, 231.45 mmol) was added as a single portion. After 3 h, the reaction was diluted with ether (800 mL) and quenched with water (400 mL) and saturated sodium bicarbonate (50 mL). The layers were separated, and the organic layer was washed with brine, dried over sodium sulfate, and concentrated under reduced pressure to yield methyl 6-benzylsulfanylpyridine-2-carboxylate (56.35 g, 89%) as a yellow oil. ¹H NMR (500 MHz, DMSO-d₆) δ 7.84-7.77 (m, 1H), 7.77-7.73 (m, 1H), 7.52 (m, 1H), 7.48 (d, J 7.8 Hz, 2H), 7.28(t, J 7.2, 7.2 Hz, 2H), 7.24-7.18 (m, 1H), 4.44 (s, 2H), 3.90 (d, J 1.2 Hz, 3H). ESI-MS m z calc. 259.0667, found 260.1 (M+1)⁺; Retention time: 3.2 minutes; LC method T.

### Step 2: Methyl 6-chlorosulfonylpyridine-2-carboxylate

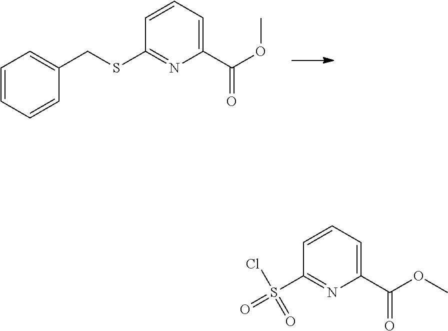 [SMILES: COC(=O)c1cccc(SCc2ccccc2)n1 ; COC(=O)c1cccc(S(=O)(=O)Cl)n1]

A solution of methyl 6-benzylsulfanylpyridine-2-carboxylate (121.62 g, 431.47 mmol) in DCM (950 mL) and DI water (300 mL) was cooled in a −1-0° C. ice bath and, with vigorous stirring, sulfuryl chloride (228.14 g, 140 mL, 1.6396 mol) was added dropwise while the temperature was maintained below 5° C. After the addition, the organic phase was separated, washed with DI water (2×500 mL), dried over anhydrous sodium sulfate, filtered and concentrated under vacuum. The residue was dissolved in DCM (500 mL). Hexanes (1000 mL) was added and the DCM was slowly evaporated off. The white precipitate was filtered by vacuum and the solids were washed with Hexanes (2×500 mL). The filtered solids were collected. The residue solids in the filtrate were filtered and dissolved in DCM (500 mL). The DCM solution was transferred to a 1 L round-bottom flask and concentrated under vacuum. The residue was dissolved in DCM (200 mL). Hexanes (600 mL) was added and the DCM was slowly evaporated off. The white precipitation was filtered by vacuum and the solids were washed with hexanes (2×500 mL) After drying, methyl 6-chlorosulfonylpyridine-2-carboxylate (56.898 g, 55%) was isolated. ¹H NMR (500 MHz, Chloroform-d) δ 8.48 (dd, J 7.8, 1.1 Hz, 1H), 8.31 (dd, J 7.9, 1.1 Hz, 1H), 8.25 (t, J 7.8 Hz, 1H), 4.08 (s, 3H). ESI-MS m/z calc. 234.97061, found 236.1 (M+1)⁺; Retention time: 1.74 minutes; LC method T.

### Step 3: Methyl 6-[[4-chloro-6-(2,6-dimethylphenyl)pyrimidin-2-yl]sulfamoyl]pyridine-2-carboxylate

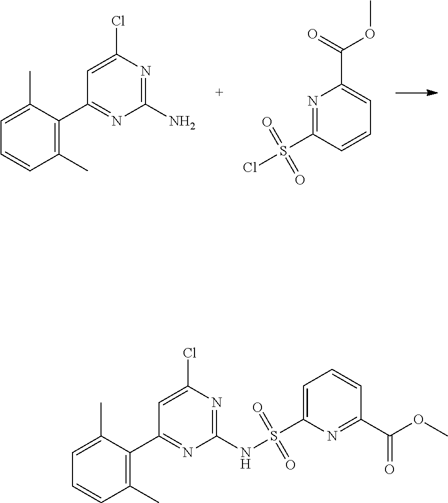 [SMILES: Cc1cccc(C)c1-c1cc(Cl)nc(N)n1 ; COC(=O)c1cccc(S(=O)(=O)Cl)n1 ; COC(=O)c1cccc(S(=O)(=O)Nc2nc(Cl)cc(-c3c(C)cccc3C)n2)n1]

A solution of 4-chloro-6-(2,6-dimethylphenyl)pyrimidin-2-amine (16.63 g, 71.161 mmol) and methyl 6-chlorosulfonylpyridine-2-carboxylate (16.8 g, 71.294 mmol) dissolved in anhydrous THE (680 mL) was cooled to −78° C. Then Lithium bis(trimethylsilyl)amide (143 mL of 1 M, 143.00 mmol) in solution in THE was added dropwise. The mixture was allowed to warm up to 0° C. slowly and then 1M aqueous HCl (146 mL) was added, followed by DI water (680 mL). The THF was evaporated and the aqueous phase was extracted with chloroform (3×250 mL). The combined organic layers were washed with saturated aqueous NaCl (300 mL), dried over anhydrous sodium sulfate, filtered, and concentrated under vacuum. The crude was recrystallized in 10% Acetone in Hexanes (500 mL). The white precipitate was filtered and rinsed with acetone (2×100 mL) to give methyl 6-[[4-chloro-6-(2,6-dimethylphenyl)pyrimidin-2-yl]sulfamoyl]pyridine-2-carboxylate (15.79 g, 50%). ESI-MS m/z calc. 432.06592, found 433.3 (M+1)⁺; Retention time: 5.5 minutes; LC method S.

### Step 4: 6-[[4-Chloro-6-(2,6-dimethylphenyl)pyrimidin-2-yl]sulfamoyl]pyridine-2-carboxylic Acid

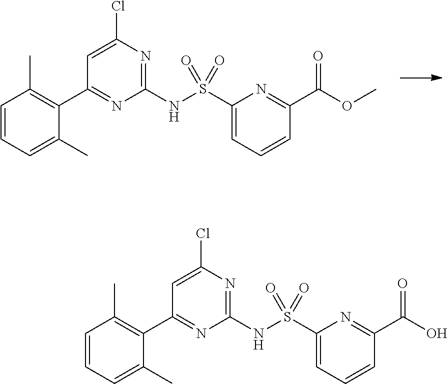 [SMILES: COC(=O)c1cccc(S(=O)(=O)Nc2nc(Cl)cc(-c3c(C)cccc3C)n2)n1 ; Cc1cccc(C)c1-c1cc(Cl)nc(NS(=O)(=O)c2cccc(C(=O)O)n2)n1]

To a solution of methyl 6-[[4-chloro-6-(2,6-dimethylphenyl)pyrimidin-2-yl]sulfamoyl]pyridine-2-carboxylate (15.79 g, 36.477 mmol) in THE (180 mL) was added aqueous sodium hydroxide (182 mL of 1 M, 182.00 mmol). The reaction was stirred at RT for 1 h. The THF was evaporated, and the aqueous layer was washed with diethyl ether (2×200 mL). The aqueous layer was acidified to pH 2 with 1 M Aqueous HCl (250 mL). The precipitate was filtered and the a white solid were rinsed with DI water (2×250 mL). The solids were dried under vacuum to give 6-[[4-chloro-6-(2,6-dimethylphenyl)pyrimidin-2-yl]sulfamoyl]pyridine-2-carboxylic acid (14.3444 g, 93%). ¹H NMR (250 MHz, DMSO-d₆) δ 8.14-7.99 (m, 3H), 7.21-7.11 (m, 1H), 7.03 (d, J 7.7 Hz, 2H), 6.92 (s, 1H), 1.78 (s, 6H). ESI-MS m/z calc. 418.05026, found 419.1 (M+1)⁺; Retention time: 2.61 minutes; LC method T.

### Example 12: Preparation of 3-[[4-[(2R)-2-amino-4-methyl-pentoxy]-6-(2,6-dimethylphenyl)-2-pyridyl]sulfamoyl]benzoic Acid

### Step 1: 4-Chloro-6-(2,6-dimethylphenyl)pyridin-2-amine

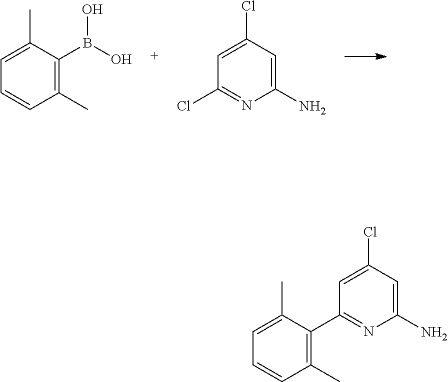 [SMILES: Cc1cccc(C)c1B(O)O ; Nc1cc(Cl)cc(Cl)n1 ; Cc1cccc(C)c1-c1cc(Cl)cc(N)n1]

To a stirring solution of (2,6-dimethylphenyl)boronic acid (11.515 g, 76.775 mmol) and 4,6-dichloropyridin-2-amine (12.513 g, 76.765 mmol) in Toluene (425 mL) and EtOH (213 mL) was added an aqueous solution of Sodium carbonate (115 mL of 2 M, 230.00 mmol) and the reaction mixture was degassed with nitrogen gas for 45 min. Pd(dppf)Cl₂ (6.271 g, 7.6791 mmol) was then added with degassing continuing for an additional 15 min. Then the reaction vial was sealed, and the mixture heated to 100° C. and stirred at that temperature for 24 h. After this time, volatiles were removed under reduced pressure and the residue was extracted with ethyl acetate (3×200 mL). The combined organic layers were washed with brine, dried over anhydrous sodium sulfate and concentrated under reduced pressure. The crude product was purified by silica gel column chromatography (0-25% EtOAc in Hexanes) and triturated with Hexanes to afford 4-chloro-6-(2,6-dimethylphenyl)pyridin-2-amine (6.469 g, 34%) as an off-white solid. ESI-MS m/z calc. 232.07672, found 233.1 (M+1)⁺; Retention time: 2.31 minutes; (LC method T).

### Step 2: Methyl 3-[[4-chloro-6-(2,6-dimethylphenyl)-2-pyridyl]sulfamoyl]benzoate

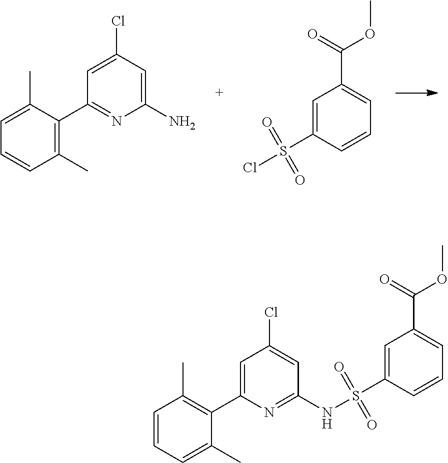 [SMILES: Cc1cccc(C)c1-c1cc(Cl)cc(N)n1 ; COC(=O)c1cccc(S(=O)(=O)Cl)c1 ; COC(=O)c1cccc(S(=O)(=O)Nc2cc(Cl)cc(-c3c(C)cccc3C)n2)c1]

To a solution of 4-chloro-6-(2,6-dimethylphenyl)pyridin-2-amine (4.9 g, 20.635 mmol) and methyl 3-chlorosulfonylbenzoate (4.9 g, 20.046 mmol) in THE (200 mL) was added dropwise Lithium bis(trimethylsilyl)amide (45 mL of 1 M, 45.000 mmol) at −78° C. under nitrogen. The reaction mixture was stirred for 30 minutes at −78° C.; then warmed up to 0° C. and stirred for 2 hours at 0° C. The reaction was quenched with cold 1.0 M Hydrochloric acid (50 mL) and diluted with water (200 mL). The mixture was extracted with ethyl acetate (2×400 mL). The organic layers were combined, washed with brine (500 mL), dried over sodium sulfate, filtered and concentrated. The residue was purified by chromatography using 0-20% ethyl acetate in hexanes to afford methyl 3-[[4-chloro-6-(2,6-dimethylphenyl)-2-pyridyl]sulfamoyl]benzoate (6.2 g, 68%) as a white solid. ESI-MS m/z calc. 430.0754, found 431.5 (M+1)⁺; Retention time: 3.65 minutes; (LC method T).

### Step 3: 3-[[4-chloro-6-(2,6-dimethylphenyl)-2-pyridyl]sulfamoyl]benzoic Acid

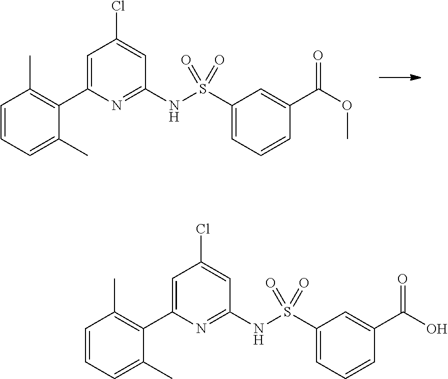 [SMILES: COC(=O)c1cccc(S(=O)(=O)Nc2cc(Cl)cc(-c3c(C)cccc3C)n2)c1 ; Cc1cccc(C)c1-c1cc(Cl)cc(NS(=O)(=O)c2cccc(C(=O)O)c2)n1]

To a stirring solution of 3-[4-chloro-6-(2,6-dimethyl-phenyl)-pyridin-2-ylsulfamoyl]-benzoic acid methyl ester (5.3 g, 12.3 mmol) in a mixture of tetrahydrofuran (80 mL) and water (80 mL) at room temperature was added lithium hydroxide monohydrate (1.55 g, 36.9 mmol) and the reaction mixture was stirred at 45 C for 2 hours. Tetrahydrofuran was removed under vacuum and the residue was diluted with water (100 mL). The aqueous layer was washed with diethyl ether (2×50 mL), hexanes (50 mL) and acidified with 1.0 M hydrochloric acid to pH=2-3. The precipitated product was collected by filtration and dried in a vacuum oven at 75 C to constant weight to afford 3-[4-chloro-6-(2,6-dimethyl-phenyl)-pyridin-2-ylsulfamoyl]-benzoic acid (4.8 g, 93%) as a white solid. H NMR (250 MHz, DMSO-d₆) δ (ppm): 8.32 (d, J=1.9 Hz, 1H), 8.14 (d, J=7.7 Hz, 1H), 8.03 (d, J=8.0 Hz, 1H), 7.63 (t, J=7.8 Hz, 1H), 7.28-6.96 (m, 5H), 1.77 (s, 6H). ESI-MS m/z calc. 416.8, found 417.0 (M1). Retention time: 5.11 minutes.

### Step 4: 3-[[4-[(2R)-2-Amino-4-methyl-pentoxy]-6-(2,6-dimethylphenyl)-2-pyridyl]sulfamoyl]benzoic Acid

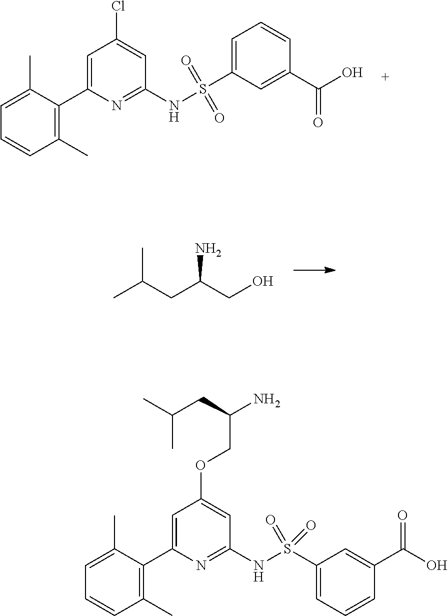

A 20 mL vial was charged with 3-[[4-chloro-6-(2,6-dimethylphenyl)-2-pyridyl]sulfamoyl]benzoic acid (300 mg, 0.7196 mmol), (2R)-2-amino-4-methyl-pentan-1-ol (110 mg, 0.9387 mmol) and anhydrous tetrahydrofuran (12 mL), in that order. Then the vial was purged with nitrogen for 30 seconds, and solid potassium tert-butoxide (350 mg, 3.119 mmol) was added capped under nitrogen. After stirred at 105° C. for 14 h (overnight), the reaction was allowed to cool to ambient temperature. Then glacial acetic acid (200 μL, 3.517 mmol) was added and the volatiles were removed under reduced pressure. To the residue, DMSO (5 mL) was added and microfiltered. Purification by reverse phase chromatography (C₁₈ column, 1-99% acetonitrile in water over 15 min) gave 3-[[4-[(2R)-2-amino-4-methyl-pentoxy]-6-(2,6-dimethylphenyl)-2-pyridyl]sulfamoyl]benzoic acid (hydrochloride salt)(278 mg, 72%) as yellowish solid. ESI-MS m/z calc. 497.19846, found 498.2 (M+1)⁺; Retention time: 0.43 minutes (LC method D).

### V. Synthesis of Compounds

### Example 13: Preparation of Compound 1

### Step 1: 3-Amino-N-[4-(3-aminophenoxy)-6-(2,6-dimethylphenyl)pyrimidin-2-yl]benzenesulfonamide

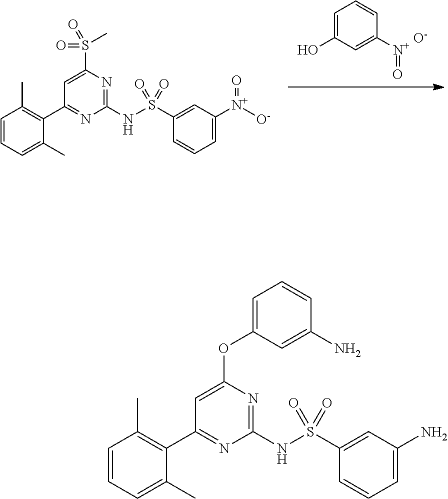

Stage 1:To a 20 mL vial equipped with a magnetic stir bar, N-[4-(2,6-dimethylphenyl)-6-methylsulfonyl-pyrimidin-2-yl]-3-nitro-benzenesulfonamide (198.3 mg, 0.4288 mmol), N-methylpyrrolidinone (5.0 mL) and 3-nitrophenol (237.7 mg, 1.709 mmol) were added, followed by potassium carbonate (240.2 mg, 1.738 mmol). This solution was stirred at 100° C. for 15 h. The reaction mixture was then cooled to room temperature, quenched with 1 N HCl (5 mL), and extracted with ethyl acetate (3×5 mL). The combined organic extracts were washed with water (2×5 mL) and saturated aqueous sodium chloride solution (5 mL), then dried over sodium sulfate, filtered, and evaporated in vacuo to give a brown oil. Purification by silica gel chromatography (12 g of silica, 0 to 40% gradient of ethyl acetate/hexanes) gave 253.3 mg of an off-white foam.

Stage 2: In a 10 mL vial equipped with a magnetic stir bar, the product from Stage 1 was dissolved in ethyl acetate (2.5 mL) and ethanol (2.5 mL), and this cloudy solution was purged with a balloon of hydrogen gas for 5 min. The cap was briefly removed, and 10% Pd(OH)₂/C (25.3 mg, 0.01802 mmol) was added. This reaction mixture was stirred under hydrogen gas (2 L, 79.37 mmol) at 70° C. for 24 h. It was then filtered through Celite and rinsed with methanol (10 mL). This solution was evaporated in vacuo to give an off-white foam, 3-amino-N-[4-(3-aminophenoxy)-6-(2,6-dimethylphenyl)pyrimidin-2-yl]benzenesulfonamide (dihydrochloride salt) (224.0 mg, 48%) ESI-MS m/z calc. 461.15216, found 462.3 (M+1)⁺; Retention time: 1.2 minutes; LC method A.

### Step 2: 5-(2,6-Dimethylphenyl)-9,9-dioxo-2-oxa-9λ⁶-thia-6,8,15,17,24-pentazatetracyclo[16.3.1.13,7.110,14]tetracosa-1(21),3,5,7(24),10(23),11,13,18(22),19-nonaen-16-one (Compound 1)

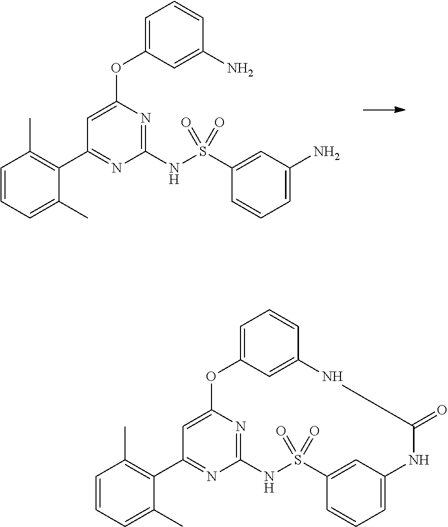 [SMILES: Cc1cccc(C)c1-c1cc(Oc2cccc(N)c2)nc(NS(=O)(=O)c2cccc(N)c2)n1 ; Cc1cccc(C)c1-c1cc2nc(n1)NS(=O)(=O)c1cccc(c1)NC(=O)Nc1cccc(c1)O2]

In a 3 mL vial equipped with a magnetic stir bar, 3-amino-N-[4-(3-aminophenoxy)-6-(2,6-dimethylphenyl)pyrimidin-2-yl]benzenesulfonamide (Dihydrochloride salt) (20.0 mg, 0.02619 mmol) was dissolved in 1,3-dimethyl-tetrahydro-pyrimidin-2-one (1.0 mL), to which carbonyl diimidazole (7.9 mg, 0.04872 mmol) was added. This solution was stirred at 90° C. for 2 h. After cooling to room temperature, this solution was filtered and purified by reverse phase HPLC (1-50% acetonitrile in water using HCl as a modifier) to give the desired 5-(2,6-dimethylphenyl)-9,9-dioxo-2-oxa-9λ⁶-thia-6,8,15,17,24-pentazatetracyclo[16.3.1.13,7.110,14]tetracosa-1(21),3,5,7(24),10(23),11,13,18(22),19-nonaen-16-one (1.7 mg, 13%) ESI-MS m/z calc. 487.13144, found 488.2 (M+1)⁺; Retention time: 1.36 minutes; LC method A.

### Example 14: Preparation of 21-(2-Methylphenoxy)-17λ⁶-thia-11,18,20,23-tetrazatetracyclo[17.3.1.112,16.02,7]tetracosa-1(22),2(7),3,5,12,14,16(24),19(23),20-nonaene 17,17-dioxide

### Step 1: 3-[2-(4,4,5,5-Tetramethyl-[1,3,2]dioxaborolan-2-yl)-phenyl]-propan-1-ol

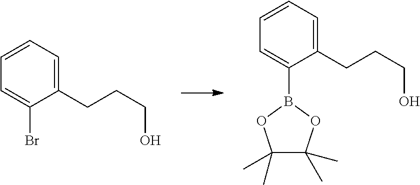 [SMILES: OCCCc1ccccc1Br ; CC1(C)OB(c2ccccc2CCCO)OC1(C)C]

3-(2-Bromo-phenyl)-propan-1-ol (4.6 g, 21.4 mmol), bis(pinacolato)diboron (16.3 g, 64.2 mmol) and potassium acetate (6.3 g, 64.2 mmol) were suspended in dimethylsulfoxide (100 mL). Dry argon bubbled through solution for several minutes and then [1,1′-bis(diphenylphosphino)ferrocene]dichloropalladium(II) (0.78 g, 1.1 mmol) was added. The reaction mixture stirred at 80° C. under argon for 16 hours. The mixture was cooled to room temperature, diluted with water (50 mL) and extracted with 1:1 mixture of hexane and diethyl ether (3×200 mL). Combined organic phases dried over sodium sulfate, filtered and concentrated under vacuum. The residue was purified by silica gel column chromatography using 0-40% hexanes-ethyl acetate to afford 3-[2-(4,4,5,5-tetramethyl-[1,3,2]dioxaborolan-2-yl)-phenyl]-propan-1-ol (3.2 g, 57%) as a colorless oil. ESI-MS m/z calc. 262.174, found 263.1 (M+1)⁺; Retention time: 4.86 minutes.

### Step 2: 3-[2-(4,4,5,5-Tetramethyl-[1,3,2]dioxaborolan-2-yl)-phenyl]-propionaldehyde

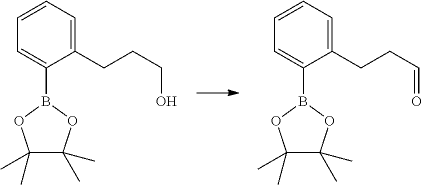 [SMILES: CC1(C)OB(c2ccccc2CCCO)OC1(C)C ; CC1(C)OB(c2ccccc2CCC=O)OC1(C)C]

Solution of 1,1,1-triacetoxy-1,1-dihydro-1,2-benziodoxol-3(1H)-one (6.2 g, 14.6 mmol) in dichloromethane (50 mL) was added to a solution of 3-[2-(4,4,5,5-tetramethyl-[1,3,2]dioxaborolan-2-yl)-phenyl]-propan-1-ol (3.2 g, 12.2 mmol) in dichloromethane (50 mL) at 0° C. The reaction mixture was allowed to warm to room temperature and stirred for 14 hours. Mixture of 0.1 M aqueous sodium metabisulfite (20 mL) and saturated sodium bicarbonate (10 mL) was added to reaction mixture and the mixture stirred for 20 minutes. Organic phase was separated and filtered through Celite. The filtrates were concentrated and the residue was purified by silica gel column chromatography using 0-35% hexanes-ethyl acetate to afford 3-[2-(4,4,5,5-tetramethyl-[1,3,2]dioxaborolan-2-yl)-phenyl]-propionaldehyde (1.6 g, 50%) as a colorless liquid. ESI-MS m/z calc. 260.2, found 261.4 (M+1)⁺. Retention time: 5.55 minutes.

### Step 3: 3-Amino-N-(4,6-dichloro-pyrimidin-2-yl)-benzenesulfonamide

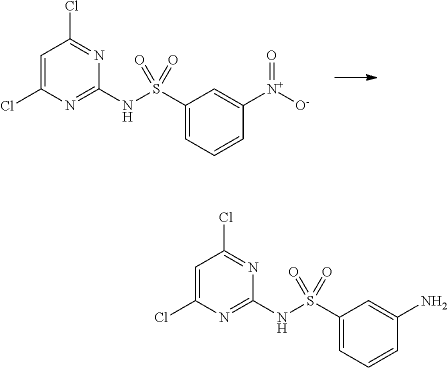 [SMILES: O=[N+]([O-])c1cccc(S(=O)(=O)Nc2nc(Cl)cc(Cl)n2)c1 ; Nc1cccc(S(=O)(=O)Nc2nc(Cl)cc(Cl)n2)c1]

To a solution of N-(4,6-dichloro-pyrimidin-2-yl)-3-nitro-benzenesulfonamie (3.0 g, 8.6 mmol) in tetrahydrofuran:ethanol:water mixture (20:20:3 mL) was added iron (2.4 g, 42.9 mmol) and 6N hydrochloric acid (7.0 mL, 42 mmol). The reaction was heated to 60° C. and stirred at this temperature for 30 minutes. After cooling to room temperature the reaction mixture was filtered through Celite, filtrates were concentrated under vacuum. The residue was purified by silica gel chromatography using 30-90% hexanes-ethyl acetate to afford 3-amino-N-(4,6-dichloro-pyrimidin-2-yl)-benzenesulfonamide (1.8 g, 66%) as a tan solid. ¹H NMR (250 MHz, DMSO-d₆) δ 7.52 (s, 1H), 7.22-7.05 (m, 3H), 6.78 (m, 1H). ESI-MS m/z calc. 318.0, found 318.8 (M+1)⁺. Retention time: 3.07 minutes.

### Step 4: N-(4,6-Dichloro-pyrimidin-2-yl)-3-{3-[2-(4,4,5,5-tetramethyl-[1,3,2]dioxaborolan-2-yl)-phenyl]-propylamino}-benzenesulfonamide

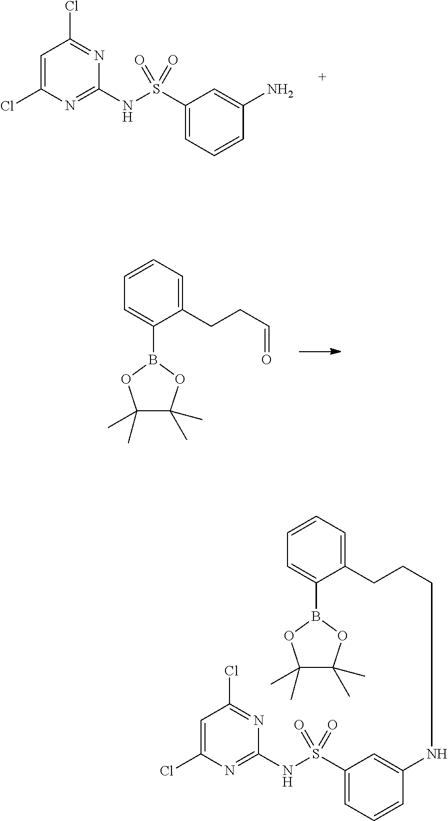 [SMILES: Nc1cccc(S(=O)(=O)Nc2nc(Cl)cc(Cl)n2)c1 ; CC1(C)OB(c2ccccc2CCC=O)OC1(C)C ; CC1(C)OB(c2ccccc2CCCNc2cccc(S(=O)(=O)Nc3nc(Cl)cc(Cl)n3)c2)OC1(C)C]

3-Amino-N-(4,6-dichloro-pyrimidin-2-yl)-benzenesulfonamide (1.9 g, 6.0 mmol), (3-[2-(4,4,5,5-tetramethyl-[1,3,2]dioxaborolan-2-yl)-phenyl]-propionaldehyde (1.6 g, 6.2 mmol) and activated molecular seives 4A (750 mg) were suspended in dry dichloromethane (50 mL). Sodium triacetoxyborohydride (1.9 g, 9.0 mmol) was added at 0° C. under argon atmosphere. The reaction mixture was allowed to warm to room temperature and stirred for 1 hour. The mixture was quenched with methanol and filtered through Celite. The filtrates were concentrated under vacuum and the residue was purified by silica gel column chromatography using 0-25% hexanes-ethyl acetate to afford N-(4,6-dichloro-pyrimidin-2-yl)-3-{3-[2-(4,4,5,5-tetramethyl-[1,3,2]dioxaborolan-2-yl)-phenyl]-propylamino}-benzenesulfonamide (1.3 g, 38%) as a white solid. ¹H NMR (250 MHz, DMSO-d₆) δ 12.35 (br.s, 1H), 7.63 (d, J 8.3 Hz, 1H), 7.51 (s, 1H), 7.40-7.00 (m, 7H), 6.75 (d, J 8.8 Hz, 1H), 6.23 (br.s, 1H), 3.05 (m, 2H), 2.91 (m, 2H), 1.78 (m, 2H), 1.25 (s, 12H). ESI-MS m/z calc. 562.1, found 563.1 (M1). Retention time: 6.87 minutes.

### Step 5: 21-chloro-17λ⁶-thia-11,18,20,23-tetraazatetracyclo[17.3.1.112,16.02,7]tetracosa-1(23),2(7),3,5,12(24),13,15,19,21-nonaene-17,17-dione

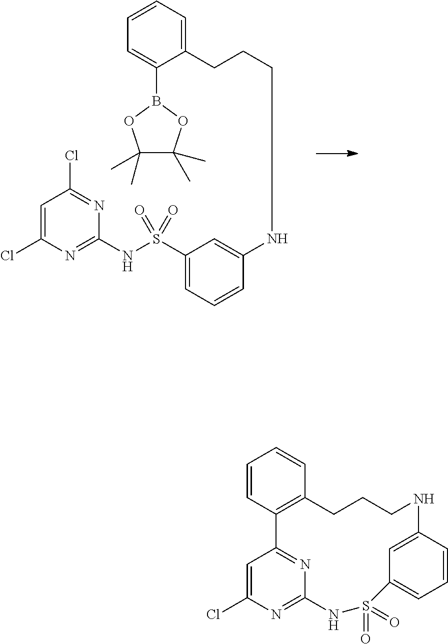 [SMILES: CC1(C)OB(c2ccccc2CCCNc2cccc(S(=O)(=O)Nc3nc(Cl)cc(Cl)n3)c2)OC1(C)C ; O=S1(=O)Nc2nc(Cl)cc(n2)-c2ccccc2CCCNc2cccc1c2]

N-(4,6-Dichloro-pyrimidin-2-yl)-3-{3-[2-(4,4,5,5-tetramethyl-[1,3,2]dioxaborolan-2-yl)-phenyl]-propylamino}-benzenesulfonamide (105 mg, 0.19 mmol) and potassium carbonate (87 mg, 0.63 mmol) were suspended in a mixture of 1,4-dioxane (15.5 mL) and water (1.5 mL). Dry argon bubbled through solution for several minutes and then tetrakis(triphenylphosphine)palladium(0) (44 mg, 0.038 mmol) was added. The reaction mixture stirred in microwave at 90° C. for 5 hours. The mixture was cooled to room temperature, diluted in ethyl acetate (50 mL) and washed with water (10 mL), brine (10 mL). The organic phase dried over sodium sulfate, filtered and concentrated under vacuum. The residue was purified by silica gel column chromatography using 0-45% hexanes-ethyl acetate to afford 21-chloro-17λ⁶-thia-11,18,20,23-tetraazatetracyclo[17.3.1.112,16.02,7]tetracosa-1(23),2(7),3,5,12(24),13,15,19,21-nonaene-17,17-dione (14 mg, 19%) as a white solid. ¹H NMR (250 MHz, DMSO-d₆) δ 11.77 (s, 1H), 7.51 (s, 1H), 7.40-7.35 (m, 4H), 7.20 (t, J=7.5 Hz, 1H), 7.10-6.90 (m, 2H), 6.77 (d, J 8.0 Hz, 1H), 6.24 (t, J 7.5 Hz, 1H), 2.90 (m, 4H), 1.26 (m, 2H). ESI-MS m/z calc. 400.1, found 401.2 (M1). Retention time: 4.89 minutes.

### Step 6: 21-(2-Methylphenoxy)-17λ⁶-thia-11,18,20,23-tetrazatetracyclo[17.3.1.112,16.02,7]tetracosa-1(22),2(7),3,5,12,14,16(24),19(23),20-nonaene 17,17-dioxide

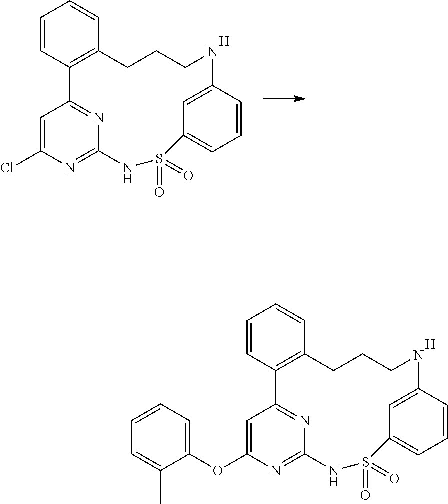

21-chloro-17λ⁶-thia-11,18,20,23-tetraazatetracyclo[17.3.1.112,16.02,7]tetracosa-1(23),2(7),3,5,12(24),13,15,19,21-nonaene-17,17-dione (25 mg, 0.06 mmol), cesium carbonate (150 mg, 0.46 mmol) and o-cresol (45 mg, 0.42 mmol) were suspended in dry acetonirtile (4 mL). The mixture was stirred in microwave at 90° C. for 12 hours. The reaction mixture was filtered, the filtrate concentrated under vacuum. The residue was purified by reverse phase HPLC using 5-100% water-acetonitrile (0.1% triflouroacetic acid) to afford 21-(2-methylphenoxy)-17λ⁶-thia-11,18,20,23-tetrazatetracyclo[17.3.1.112,16.02,7]tetracosa-1(22),2(7),3,5,12,14,16(24),19(23),20-nonaene 17,17-dioxide (20 mg, 71%) as a white solid. ¹H NMR (250 MHz, DMSO-d₆) δ 11.33 (s, 1H), 7.45-7.15 (m, 8H), 7.10-6.95 (m, 2H), 6.87 (d, J 7.3 Hz, 1H), 6.76 (d, J 8.0 Hz, 1H), 6.60 (s, 1H), 2.91 (m, 4H), 1.94 (s, 3H), 1.29 (m, 2H). ESI-MS m/z calc. 472.2, found 473.2 (M1). Retention time: 2.71 minutes.

### Example 15: Preparation of Compound 3

### Step 1: N-[2-(4-tert-butylphenyl)-2-hydroxy-ethyl]-3-[[4-chloro-6-(2,6-dimethylphenyl)pyrimidin-2-yl]sulfamoyl]benzamide

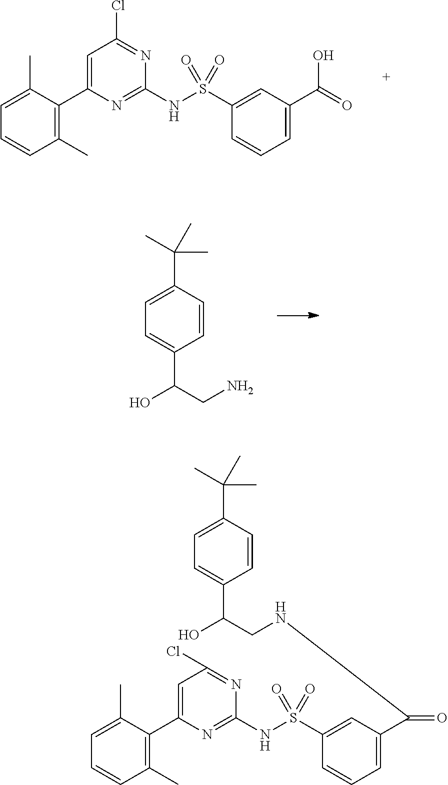 [SMILES: Cc1cccc(C)c1-c1cc(Cl)nc(NS(=O)(=O)c2cccc(C(=O)O)c2)n1 ; CC(C)(C)c1ccc(C(O)CN)cc1 ; Cc1cccc(C)c1-c1cc(Cl)nc(NS(=O)(=O)c2cccc(C(=O)NCC(O)c3ccc(C(C)(C)C)cc3)c2)n1]

In a 50-mL round-bottomed flask, 3-[[4-chloro-6-(2,6-dimethylphenyl)pyrimidin-2-yl]sulfamoyl]benzoic acid (1.050 g, 2.513 mmol), 2-amino-1-(4-tert-butylphenyl)ethanol (0.5009 g, 2.592 mmol), potassium carbonate (0.908 g, 6.570 mmol) and DCM (10 mL) were mixed together. DIC (0.80 mL, 5.109 mmol) was then added, and this mixture was stirred at room temperature for 1 h. It was then quenched with 1 N HCl solution (15 mL), diluted with water (30 mL), and extracted with dichloromethane (3×40 mL). The combined organic extracts were washed with water (100 mL) and saturated aqueous sodium chloride solution (100 mL), then dried over sodium sulfate, filtered, and evaporated in vacuo. The resulting yellow oil was purified by silica gel chromatography (80 g of silica) using a gradient eluent of 1 to 70% ethyl acetate in hexanes to give two batches of product (both white foams): 236.9 mg impure product, consisting of 24% activated ester intermediate (˜57 mg)+76% product (˜180 mg); and 775.2 mg product. The total amount of product should be 955 mg but keeping into account a 10:4 ratio of product to residual ethyl acetate observed by ¹H NMR (i.e., the product is only 94% w/w of the entire product sample), the mass of the product was reduced accordingly: N-[2-(4-tert-butylphenyl)-2-hydroxy-ethyl]-3-[[4-chloro-6-(2,6-dimethylphenyl)pyrimidin-2-yl]sulfamoyl]benzamide (0.898 g, 60%). ¹H NMR (400 MHz, dimethylsulfoxide-d₆) δ 12.41 (s, 1H, D₂O exchangeable), 8.77 (t, J 5.7 Hz, 1H, D₂O exchangeable), 8.37 (t, J 1.8 Hz, 1H), 8.10 (dt, J 7.8, 1.3 Hz, 1H), 8.03 (ddd, J 7.9, 2.0, 1.1 Hz, 1H), 7.62 (t, J 7.9 Hz, 1H), 7.38-7.34 (m, 2H), 7.29 (s, 1H), 7.29-7.26 (m, 2H), 7.23 (dd, J 8.1, 7.1 Hz, 1H), 7.09 (d, J 7.4 Hz, 2H), 5.69-5.20 (bs, 1H, D₂O exchangeable), 4.74 (dd, J 8.0, 4.8 Hz, 1H), 3.44 (ddd, J 13.2, 5.9, 4.7 Hz, 1H), 3.35-3.24 (m, 1H, hidden under water peak), 1.83 (s, 6H), 1.27 (s, 9H). ESI-MS m/z calc. 592.1911, found 593.4 (M+1)⁺; Retention time: 1.98 minutes; LC method A.

### Step 2: 10-(4-tert-butylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 3)

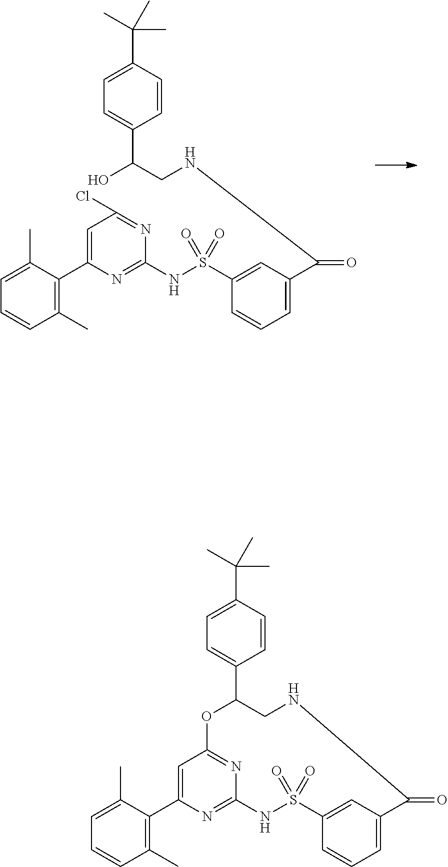 [SMILES: Cc1cccc(C)c1-c1cc(Cl)nc(NS(=O)(=O)c2cccc(C(=O)NCC(O)c3ccc(C(C)(C)C)cc3)c2)n1 ; Cc1cccc(C)c1-c1cc2nc(n1)NS(=O)(=O)c1cccc(c1)C(=O)NCC(c1ccc(C(C)(C)C)cc1)O2]

In a 100-mL round-bottomed flask, N-[2-(4-tert-butylphenyl)-2-hydroxy-ethyl]-3-[[4-chloro-6-(2,6-dimethylphenyl)pyrimidin-2-yl]sulfamoyl]benzamide (201.1 mg, 0.3390 mmol) was dissolved in NMP (40 mL), treated with NaH (82.4 mg of 60% w/w, 2.060 mmol), and stirred at 90° C. for 2.5 h. The reaction mixture was then cooled to room temperature, quenched with 1 N HCl (5 mL), diluted with water (50 mL), and extracted with ethyl acetate (3×40 mL). The combined organic extracts were washed with water (150 mL) and saturated aqueous sodium chloride solution (150 mL), then dried over sodium sulfate, filtered, and evaporated in vacuo. The resulting oil (still contains NMP) was diluted with MeOH (1 mL), filtered, and purified by reverse phase preparative chromatography using a C₁₈ column and a gradient eluent of 1 to 70% acetonitrile in water containing 5 mM hydrochloric acid to give 10-(4-tert-butylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (21.7 mg, 12%); ¹H NMR (400 MHz, chloroform-d) δ 8.85 (s, 1H), 7.85 (dt, J 7.7, 1.4 Hz, 1H), 7.82 (d, J 8.5 Hz, 1H), 7.59 (t, J 7.8 Hz, 1H), 7.52 (s, 4H), 7.25 (t, J 7.6 Hz, 1H), 7.07 (d, J 7.6 Hz, 2H), 6.26 (dd, J 10.8, 4.2 Hz, 1H), 6.23 (s, 1H), 5.46 (dd, J 10.9, 4.7 Hz, 1H), 3.98 (ddd, J 13.8, 11.0, 4.2 Hz, 1H), 3.32 (ddd, J 13.8, 10.8, 4.7 Hz, 1H), 2.05 (s, 6H), 1.37 (s, 9H). ESI-MS m/z calc. 556.2144, found 557.4 (M+1)⁺; Retention time: 1.82 minutes; LC method A.

### Example 16: Preparation of Compound 4 and Compound 5

### Step 1: (10S)-10-(4-tert-butylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 4), and (10R)-10-(4-tert-butylphenyl)-6-(2,6-dimethylphenyl)-9-oxa-2λ⁶-thia-3,5,12,19-tetraazatricyclo[12.3.1.14,8]nonadeca-1(18),4,6,8(19),14,16-hexaene-2,2,13-trione (Compound 5)

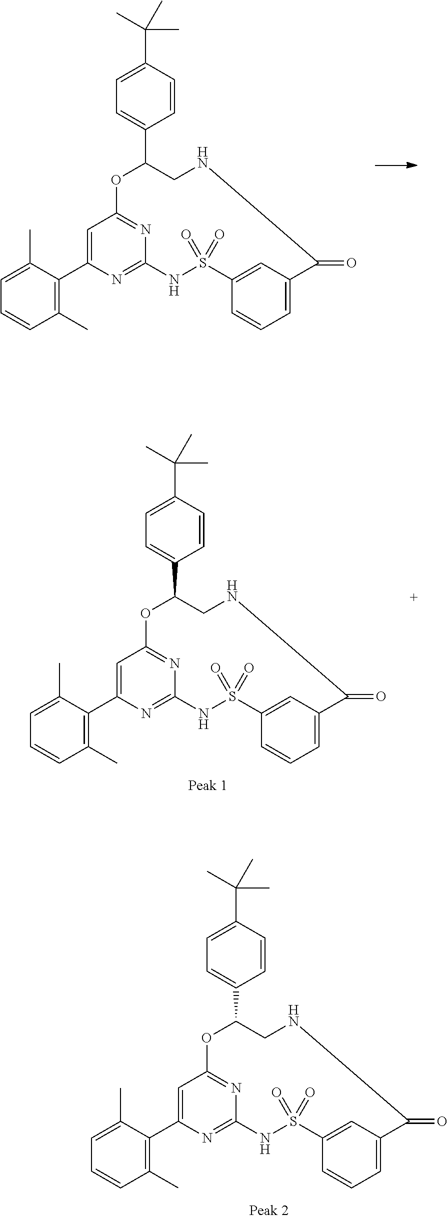 [SMILES: Cc1cccc(C)c1-c1cc2nc(n1)NS(=O)(=O)c1cccc(c1)C(=O)NCC(c1ccc(C(C)(C)C)cc1)O2 ; Cc1cccc(C)c1-c1cc2nc(n1)NS(=O)(=O)c1cccc(c1)C(=O)NC[C@H](c1ccc(C(C)(C)C)cc1)O2 ; Cc1cccc(C)c1-c1cc2nc(n1)NS(=O)(=O)c1cccc(c1)C(=O)NC[C@@H](c1ccc(C(C)(C)C)cc1)O2]

In a 20-mL vial, 10-(4-tert-butylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (20.9 mg, 0.03754 mmol) was dissolved in 17:3 MeOH:DMSO (ca. 1 mL) to achieve a concentration of ca. 24 mg/mL. Separation of the enantiomers was achieved with an SFC purification method using a ChiralPak AS-H column (250×10 mm, 5 m particle size), with a mobile phase of 30% MeOH+70% CO₂, a flow rate of 10 mL/min, an injection volume of 70 μL, and a pressure of 100 bar. The collected batches were labeled “Peak 1” (6.4 mg, 31%) and “Peak 2” (6.3 mg, 30%). These compounds were diluted with 1:1 DMSO:MeOH (1 mL), filtered, and purified by reverse phase preparative chromatography using a C₁₈ column and a gradient eluent of 1 to 99% acetonitrile in water containing 5 mM hydrochloric acid to give two products. “Peak 1” was (10S)-10-(4-tert-butylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.5 mg, 26%). ESI-MS m/z calc. 556.2144, found 557.4 (M+1)⁺; Retention time: 1.82 minutes; LC method A. Retention time: 2.07 minutes (chiral AS-3 5-min column). “Peak 2” was (10R)-10-(4-tert-butylphenyl)-6-(2,6-dimethylphenyl)-9-oxa-2λ⁶-thia-3,5,12,19-tetraazatricyclo[12.3.1.14,8]nonadeca-1(18),4,6,8(19),14,16-hexaene-2,2,13-trione (5.1 mg, 24%). ¹H NMR (400 MHz, chloroform-d) δ 8.86 (t, J 1.6 Hz, 1H), 7.88 (d, J 7.6 Hz, 1H), 7.84 (d, J 7.9 Hz, 1H), 7.63 (t, J 7.8 Hz, 1H), 7.52 (s, 4H), 7.25 (t, J 7.6 Hz, 1H), 7.07 (d, J 7.6 Hz, 2H), 6.26 (dd, J 10.8, 4.2 Hz, 1H), 6.25 (s, 1H), 5.42 (dd, J 10.8, 4.6 Hz, 1H), 3.99 (ddd, J 14.4, 11.4, 4.1 Hz, 2H), 3.33 (ddd, J 14.6, 10.6, 4.6 Hz, 1H), 2.05 (s, 6H), 1.37 (s, 9H). ESI-MS m/z calc. 556.2144, found 557.4 (M+1)⁺; Retention time: 1.83 minutes; LC method A. Retention time: 2.42 minutes (chiral AS-3 5-min column).

### Example 17: Preparation of Compound 6

### Step 1: tert-Butyl N-[2-(5-tert-butyl-2-pyridyl)-2-oxo-ethyl]carbamate

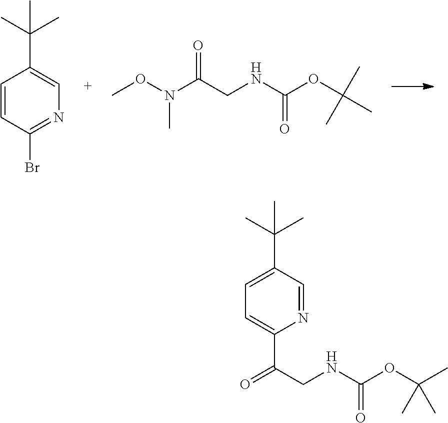 [SMILES: CC(C)(C)c1ccc(Br)nc1 ; CON(C)C(=O)CNC(=O)OC(C)(C)C ; CC(C)(C)OC(=O)NCC(=O)c1ccc(C(C)(C)C)cn1]

In a 250-mL flask, 2-bromo-5-tert-butyl-pyridine (2.12 g, 9.902 mmol) was dissolved in anhydrous THE (40 mL) and cooled to −78° C. and purged with nitrogen. A solution of n-BuLi (9.2 mL of 2.5 M, 23.00 mmol) in hexanes was added in one portion, and this mixture was stirred at −78° C. for 10 min. A solution of tert-butyl N-[2-[methoxy(methyl)amino]-2-oxo-ethyl]carbamate (2.0 g, 9.164 mmol) in anhydrous THE (10 mL) was then added in one portion. This solution was stirred at −78° C. for 5 min, and warmed to room temperature over 1 h. The reaction mixture was then quenched with 1 N citric acid (25 mL), neutralized with saturated aqueous sodium bicarbonate solution (50 mL), and extracted with ethyl acetate (2×50 mL). The combined organic extracts were washed with water (50 mL) and saturated aqueous sodium chloride solution (50 mL), then dried over sodium sulfate, filtered, and evaporated in vacuo. The resulting orange oil was purified by silica gel chromatography (120 g of silica) using a gradient eluent of 100% hexanes to 20% ethyl acetate in hexanes to give the product as a light yellow oil, tert-butyl N-[2-(5-tert-butyl-2-pyridyl)-2-oxo-ethyl]carbamate (1.5 g, 56%). ESI-MS m/z calc. 292.17868, found 293.3 (M+1)⁺; Retention time: 1.66 minutes; LC method A.

### Step 2: tert-Butyl N-[2-(5-tert-butyl-2-pyridyl)-2-hydroxy-ethyl]carbamate

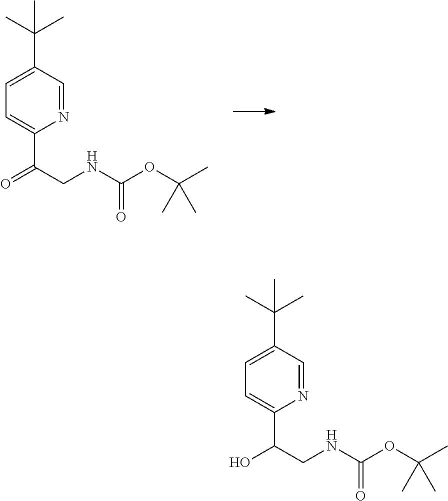 [SMILES: CC(C)(C)OC(=O)NCC(=O)c1ccc(C(C)(C)C)cn1 ; CC(C)(C)OC(=O)NCC(O)c1ccc(C(C)(C)C)cn1]

In a 250-mL flask, tert-butyl N-[2-(5-tert-butyl-2-pyridyl)-2-oxo-ethyl]carbamate (1.5 g, 5.130 mmol) was dissolved in MeOH (36 mL), to which sodium borohydride (300 mg, 7.930 mmol) was added. This mixture was stirred at room temperature for 5 min. Then, it was quenched with 1 N citric acid solution (10 mL). The mixture was neutralized with saturated aqueous sodium bicarbonate solution (10 mL), then extracted with ethyl acetate (2×50 mL). The combined organic extracts were washed with water (30 mL) and saturated aqueous sodium chloride solution (50 mL), then dried over sodium sulfate, filtered, and evaporated in vacuo. This product was re-dissolved in acetonitrile/DCM (50 mL) and evaporated to dryness under high vacuum to give a yellow viscous gum, tert-butyl N-[2-(5-tert-butyl-2-pyridyl)-2-hydroxy-ethyl]carbamate (1.09 g, 72%). ESI-MS m/z calc. 294.19434, found 295.3 (M+1)⁺; Retention time: 0.9 minutes; LC method A.

### Step 3: 2-Amino-1-(5-tert-butyl-2-pyridyl)ethanol

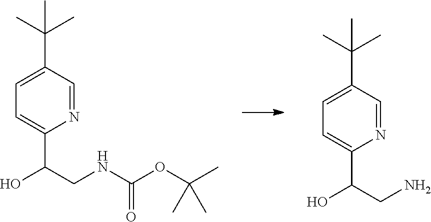 [SMILES: CC(C)(C)OC(=O)NCC(O)c1ccc(C(C)(C)C)cn1 ; CC(C)(C)c1ccc(C(O)CN)nc1]

tert-Butyl N-[2-(5-tert-butyl-2-pyridyl)-2-hydroxy-ethyl]carbamate (1.09 g, 3.703 mmol) was dissolved in DCM (30 mL) and to the mixture was added HCl (4M in dioxane) (10 mL of 4 M, 40.00 mmol) and stirred at room temperature for 90 min. The reaction mixture was concentrated under reduced pressure to a white solid, which was then slurried in diethyl ether (2×75 mLs). The solid was collected by vacuum filtration which afforded a hygroscopic gum. This material was then dissolved in dichloromethane, and evaporated in vacuo to provide as an off-white solid 2-amino-1-(5-tert-butyl-2-pyridyl)ethanol (hydrochloride salt) (850 mg, 99%) ESI-MS m/z calc. 194.1419, found 195.2 (M+1)⁺; Retention time: 0.45 minutes; LC method A.

### Step 4: 3-[[4-[2-(tert-Butoxycarbonylamino)-1-(5-tert-butyl-2-pyridyl)ethoxy]-6-(2,6-dimethylphenyl)pyrimidin-2-yl]sulfamoyl]benzoic Acid

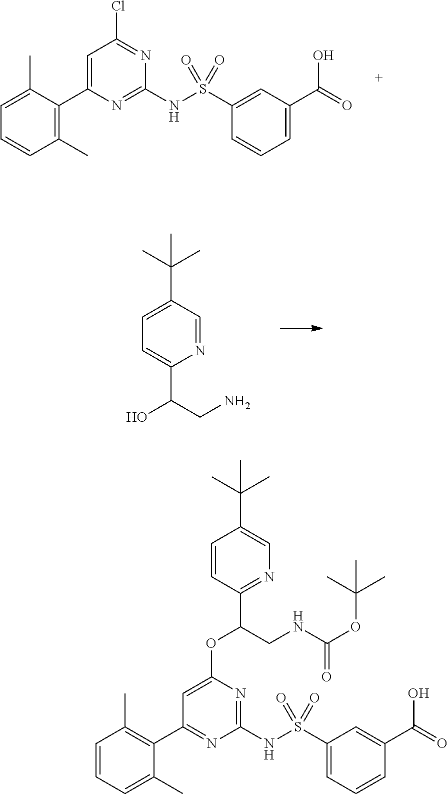 [SMILES: Cc1cccc(C)c1-c1cc(Cl)nc(NS(=O)(=O)c2cccc(C(=O)O)c2)n1 ; CC(C)(C)c1ccc(C(O)CN)nc1 ; Cc1cccc(C)c1-c1cc(OC(CNC(=O)OC(C)(C)C)c2ccc(C(C)(C)C)cn2)nc(NS(=O)(=O)c2cccc(C(=O)O)c2)n1]

In a 500 mL flask, 3-[[4-chloro-6-(2,6-dimethylphenyl)pyrimidin-2-yl]sulfamoyl]benzoic acid (1.539 g, 3.683 mmol), 2-amino-1-(5-tert-butyl-2-pyridyl)ethanol (hydrochloride salt) (850 mg, 3.684 mmol) and THE (35 mL) were mixed and cooled in an ice bath at 0° C., to which KOtBu (3.9 g, 34.76 mmol) was added. This mixture was stirred 30 min at 0° C. Then, di-tert-butyl dicarbonate (2.2 g, 10.08 mmol) was added allowed to stir for 2 hours. The mixture was then diluted with ethyl acetate and quenched with sat ammonium chloride solution and then extracted with additional ethyl acetate (3×75 mL). The combined organic extract was washed with water (50 mL) and saturated brine solution (50 mL), then dried over sodium sulfate, filtered, and evaporated in vacuo. This crude product was purified on silica gel chromatography (120 gram column) using a gradient from 100% dichloromethane to 20% methanol in dichloromethane followed by a second silica gel chromatography (80 gram column) using a gradient from 100% dichloromethane to 12% methanol in dichloromethane to afford a white solid, 3-[[4-[2-(tert-butoxycarbonylamino)-1-(5-tert-butyl-2-pyridyl)ethoxy]-6-(2,6-dimethylphenyl)pyrimidin-2-yl]sulfamoyl]benzoic acid (1.2 g, 48%) ESI-MS m/z calc. 675.27264, found 676.5 (M+1)⁺; Retention time: 1.59 minutes; LC method A.

### Step 5: 3-[[4-[2-Amino-1-(5-tert-butyl-2-pyridyl)ethoxy]-6-(2,6-dimethylphenyl)pyrimidin-2-yl]sulfamoyl]benzoic Acid

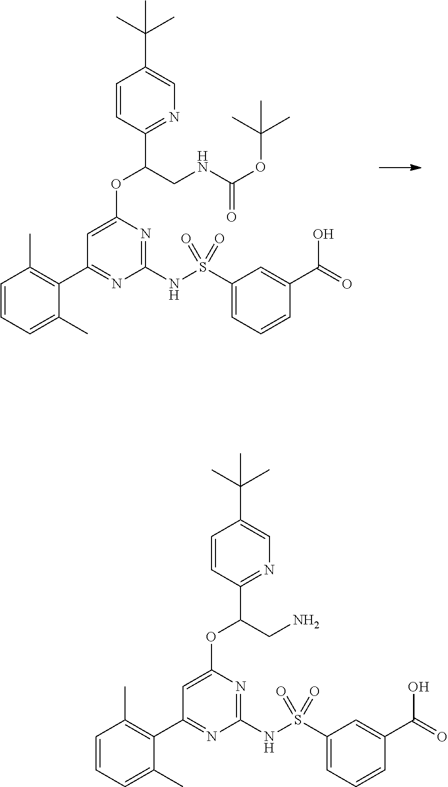 [SMILES: Cc1cccc(C)c1-c1cc(OC(CNC(=O)OC(C)(C)C)c2ccc(C(C)(C)C)cn2)nc(NS(=O)(=O)c2cccc(C(=O)O)c2)n1 ; Cc1cccc(C)c1-c1cc(OC(CN)c2ccc(C(C)(C)C)cn2)nc(NS(=O)(=O)c2cccc(C(=O)O)c2)n1]

3-[[4-[2-(tert-Butoxycarbonylamino)-1-(5-tert-butyl-2-pyridyl)ethoxy]-6-(2,6-dimethylphenyl)pyrimidin-2-yl]sulfamoyl]benzoic acid (1.2 g, 1.776 mmol) was dissolved in DCM (30 mL) and to the mixture was added HCl in dioxane (5.0 mL of 4 M, 20.00 mmol) and stirred at room temperature for 90 min. The reaction mixture was concentrated under reduced pressure to a yellow solid, which was then slurried in diethyl ether (2×20 mLs). The solid was collected by vacuum filtration to provide as a light yellow solid 3-[[4-[2-amino-1-(5-tert-butyl-2-pyridyl)ethoxy]-6-(2,6-dimethylphenyl)pyrimidin-2-yl]sulfamoyl]benzoic acid (hydrochloride salt) (0.85 g, 78%). ESI-MS m/z calc. 575.2202, found 576.3 (M+1)⁺; Retention time: 1.22 minutes; LC method A.

### Step 6: 10-(5-tert-Butyl-2-pyridyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 6)

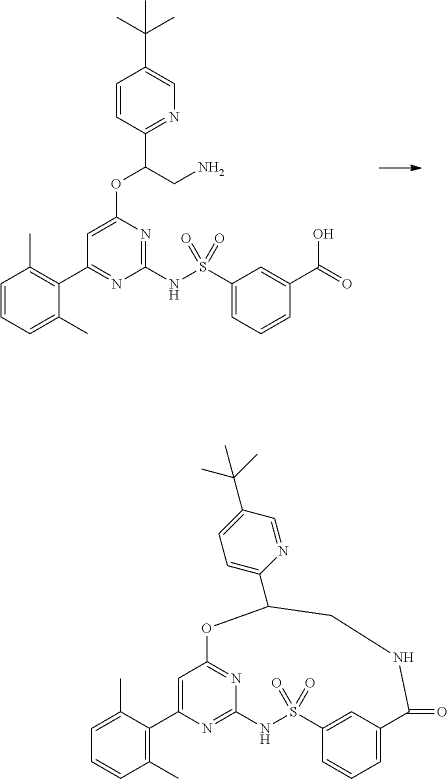 [SMILES: Cc1cccc(C)c1-c1cc(OC(CN)c2ccc(C(C)(C)C)cn2)nc(NS(=O)(=O)c2cccc(C(=O)O)c2)n1 ; Cc1cccc(C)c1-c1cc2nc(n1)NS(=O)(=O)c1cccc(c1)C(=O)NCC(c1ccc(C(C)(C)C)cn1)O2]

In a 100-mL flask, 3-[[4-[2-amino-1-(5-tert-butyl-2-pyridyl)ethoxy]-6-(2,6-dimethylphenyl)pyrimidin-2-yl]sulfamoyl]benzoic acid (hydrochloride salt) (850 mg, 1.389 mmol) was dissolved in DMF (34.0 mL), to which DIPEA (2.1 mL, 12.06 mmol) and HATU (805 mg, 2.117 mmol) were added. After stirring at room temperature for 15 min, the mixture was diluted with ethyl acetate and quenched with sat ammonium chloride solution and then extracted with additional ethyl acetate (3×75 mL). The combined organic extract was washed with water (50 mL) and saturated brine solution (50 mL), then dried over sodium sulfate, filtered, and evaporated in vacuo. This crude product was purified on silica gel chromatography (40 gram column) using a gradient from 100% dichloromethane to 20% methanol in dichloromethane followed by a second purification using reverse-phase preparative chromatography utilizing reverse-phase HPLC to afford a white solid, 10-(5-tert-butyl-2-pyridyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (272.4 mg, 33%). ¹H NMR (400 MHz, DMSO-d₆) δ 8.82 (d, J 2.5 Hz, 1H), 8.70 (s, 1H), 8.15 (d, J 8.2 Hz, 1H), 8.04-7.92 (m, 1H), 7.82 (d, J 8.3 Hz, 1H), 7.79-7.64 (m, 3H), 7.25 (t, J 7.6 Hz, 1H), 7.12 (d, J 7.7 Hz, 2H), 6.39 (dd, J 16.9, 9.3 Hz, 2H), 3.83 (s, 1H), 3.57 (t, J 7.5 Hz, 2H), 2.05 (s, 6H), 1.39 (s, 9H). ESI-MS m/z calc. 557.20966, found 558.4 (M+1)⁺; Retention time: 1.48 minutes; LC method A.

### Example 18: Preparation of Compound 7 and Compound 8

### Step 1: 10-(5-tert-Butyl-2-pyridyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, SFC peak 1 (Compound 7), and 10-(5-tert-butyl-2-pyridyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, SFC peak 2 (Compound 8)

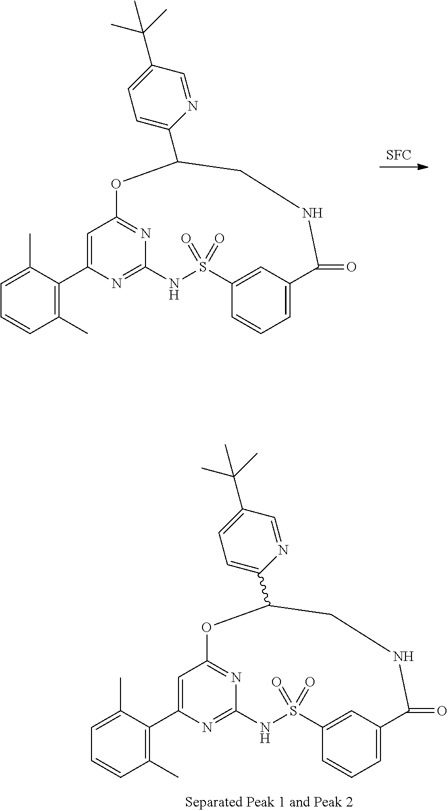 [SMILES: Cc1cccc(C)c1-c1cc2nc(n1)NS(=O)(=O)c1cccc(c1)C(=O)NCC(c1ccc(C(C)(C)C)cn1)O2 ; "SFC" ; Cc1cccc(C)c1-c1cc2nc(n1)NS(=O)(=O)c1cccc(c1)C(=O)NCC(c1ccc(C(C)(C)C)cn1)O2]

Racemic 10-(5-tert-butyl-2-pyridyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (260 mg, 0.4332 mmol) was separated into the individual enantiomers using chiral SFC (IA column): SFC Peak 1: 10-(5-tert-butyl-2-pyridyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (33.8 mg, 28%); ESI-MS m/z calc. 557.20966, found 558.4 (M+1)⁺; Retention time: 1.48 minutes; LC method A; and SFC Peak 2: 10-(5-tert-butyl-2-pyridyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (40.7 mg, 34%); ESI-MS m/z calc. 557.20966, found 558.4 (M+1)⁺; Retention time: 1.48 minutes; LC method A.

### Example 19: Preparation of Compound 9, Compound 10, and Compound 11

### Step 1: 2-Amino-1-[4-[1-(trifluoromethyl)cyclopropyl]phenyl]ethanol

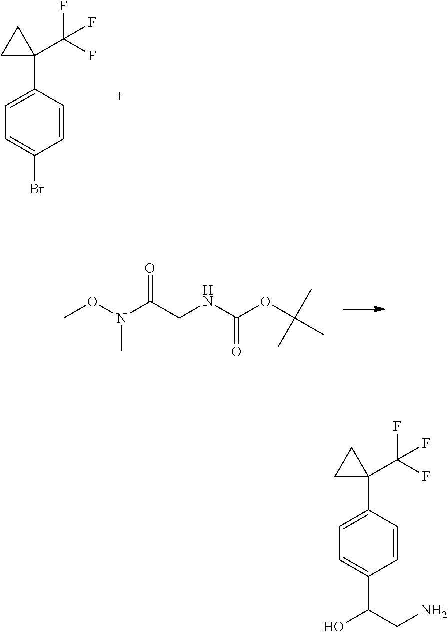 [SMILES: FC(F)(F)C1(c2ccc(Br)cc2)CC1 ; CON(C)C(=O)CNC(=O)OC(C)(C)C ; NCC(O)c1ccc(C2(C(F)(F)F)CC2)cc1]

Stage 1: To a solution of 1-bromo-4-[1-(trifluoromethyl)cyclopropyl]benzene (1.06 g, 3.999 mmol) in THE (20 mL) was treated with chloro(isopropyl)magnesium; chlorolithium (3.1 mL of 1.3 M, 4.030 mmol) at 0° C. The mixture was stirred at this temperature for 1 h and a solution of tert-butyl N-[2-[methoxy(methyl)amino]-2-oxo-ethyl]carbamate (350 mg, 1.604 mmol) in THE (10 mL) was added dropwise. The mixture was stirred at RT for 1 h and quenched with ammonium chloride, extracted with EtOAc and organic phase was dried over sodium sulfate and evaporated. The residue was purified by Silica Gel chromatography using 12 g column (eluent hexanes-EtOAc 100-0% to 80-20%) to give tert-butyl N-[2-oxo-2-[4-[1-(trifluoromethyl)cyclopropyl]phenyl]ethyl]carbamate (230 mg, 42%) ESI-MS m/z calc. 343.13953, found 244.1 (M+1)⁺; Retention time: 0.71 minutes; LC method D.

Stage 2: The obtained ketone was dissolved in MeOH (5 mL) and treated with sodium borohydride (32 mg, 0.8458 mmol) at 0° C. and stirred at RT for 1 h. The mixture was evaporated, quenched with aqueous sodium bicarbonate and extracted with EtOAc. Organic phase was dried over sodium sulfate, evaporated and purified by Silica Gel chromatography using 12 g column (eluent hexanes-EtOAc 100-0% to 80-20%) to give tert-butyl N-[2-hydroxy-2-[4-[1-(trifluoromethyl)cyclopropyl]phenyl]ethyl]carbamate (230 mg, 42%) ESI-MS m/z calc. 345.15518, found 346.27 (M+1)⁺; Retention time: 0.66 minutes; LC method D.

Stage 3: The obtained intermediate was treated with a dioxane solution of HCl (8 mL of 4 M, 32.00 mmol) and stirred at RT for 1 h. The mixture was evaporated in vacuo to give 2-amino-1-[4-[1-(trifluoromethyl)cyclopropyl]phenyl]ethanol (hydrochloride salt) (180 mg, 40%), which was used in the next step without purification. ESI-MS m/z calc. 245.10275, found 246.16 (M+1)⁺; Retention time: 0.39 minutes; LC method D.

### Step 2: 3-[[4-[2-Amino-1-[4-[1-(trifluoromethyl)cyclopropyl]phenyl]ethoxy]-6-(2,6-dimethylphenyl)pyrimidin-2-yl]sulfamoyl]benzoic Acid

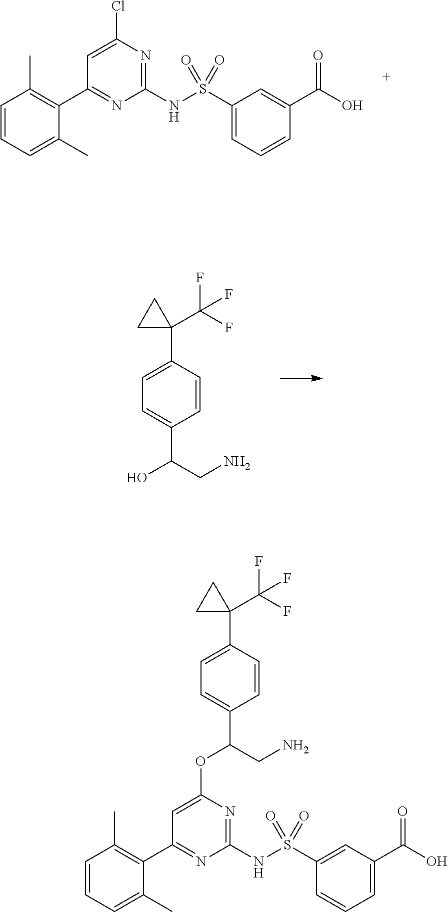 [SMILES: Cc1cccc(C)c1-c1cc(Cl)nc(NS(=O)(=O)c2cccc(C(=O)O)c2)n1 ; NCC(O)c1ccc(C2(C(F)(F)F)CC2)cc1 ; Cc1cccc(C)c1-c1cc(OC(CN)c2ccc(C3(C(F)(F)F)CC3)cc2)nc(NS(=O)(=O)c2cccc(C(=O)O)c2)n1]

A solution of 2-amino-1-[4-[1-(trifluoromethyl)cyclopropyl]phenyl]ethanol (hydrochloride salt) (180 mg, 0.6390 mmol) and 3-[[4-chloro-6-(2,6-dimethylphenyl)pyrimidin-2-yl]sulfamoyl]benzoic acid (294 mg, 0.7036 mmol) in THF (5 mL) was cooled in an ice bath. Next, sodium t-butoxide (307 mg, 3.194 mmol) was added and the mixture was stirred at RT for 3 h. NaH (51 mg of 60% w/w, 1.275 mmol) was added and the mixture was stirred at 50° C. for 30 min. The mixture was evaporated, dissolved in MeOH and purified by preparative reverse-phase HPLC (C₁₈) to afford 3-[[4-[2-amino-1-[4-[1-(trifluoromethyl)cyclopropyl]phenyl]ethoxy]-6-(2,6-dimethylphenyl)pyrimidin-2-yl]sulfamoyl]benzoic acid (hydrochloride salt) (133.7 mg, 32%) ESI-MS m/z calc. 626.1811, found 627.3 (M+1)⁺; Retention time: 0.55 minutes; LC method D.

### Step 3: 6-(2,6-Dimethylphenyl)-10-{4-[1-(trifluoromethyl)cyclopropyl]phenyl}-9-oxa-2)⁶-thia-3,5,12,19-tetraazatricyclo[12.3.1.14,8]nonadeca-1(18),4(19),5,7,14,16-hexaene-2,2,13-trione, racemic mixture (Compound 9), 6-(2,6-dimethylphenyl)-2,2-dioxo-10-[4-[1-(trifluoromethyl)cyclopropyl]phenyl]-9-oxa-2λ⁶-thia-3,5,12,19-tetrazatricyclo[12.3.1.14,8]nonadeca-1(18),4(19),5,7,14,16-hexaen-13-one, SFC peak 1 (Compound 10), and 6-(2,6-dimethylphenyl)-2,2-dioxo-10-[4-[1-(trifluoromethyl)cyclopropyl]phenyl]-9-oxa-2λ⁶-thia-3,5,12,19-tetrazatricyclo[12.3.1.14,8]nonadeca-1(18),4(19),5,7,14,16-hexaen-13-one, SFC peak 2 (Compound 11)

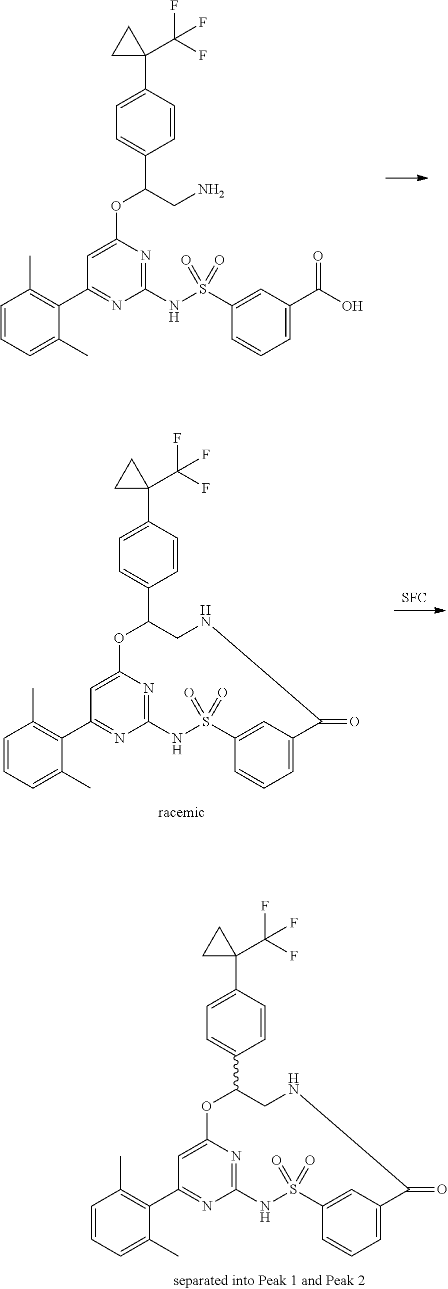 [SMILES: Cc1cccc(C)c1-c1cc(OC(CN)c2ccc(C3(C(F)(F)F)CC3)cc2)nc(NS(=O)(=O)c2cccc(C(=O)O)c2)n1 ; Cc1cccc(C)c1-c1cc2nc(n1)NS(=O)(=O)c1cccc(c1)C(=O)NCC(c1ccc(C3(C(F)(F)F)CC3)cc1)O2 ; "SFC" ; Cc1cccc(C)c1-c1cc2nc(n1)NS(=O)(=O)c1cccc(c1)C(=O)NCC(c1ccc(C3(C(F)(F)F)CC3)cc1)O2]

A solution of 3-[[4-[2-amino-1-[4-[1-(trifluoromethyl)cyclopropyl]phenyl]ethoxy]-6-(2,6-dimethylphenyl)pyrimidin-2-yl]sulfamoyl]benzoic acid (hydrochloride salt) (216 mg, 0.3257 mmol) and HATU (186 mg, 0.4892 mmol) in DMF (4 mL) was cooled in an ice bath. DIPEA (170 μL, 0.9760 mmol) was added and the mixture was stirred at RT for 1 h, filtered and purified by preparative reverse phase HPLC (C₁₈) to afford racemic 6-(2,6-dimethylphenyl)-10-{4-[1-(trifluoromethyl)cyclopropyl]phenyl}-9-oxa-2λ⁶-thia-3,5,12,19-tetraazatricyclo[12.3.1.14,8]nonadeca-1(18),4(19),5,7,14,16-hexaene-2,2,13-trione. ESI-MS m z calc. 608.17053, found 609.29 (M+1)⁺; Retention time: 1.83 minutes; LC method A.

Some of the product was kept aside (2.7 mg), and the remainder was purified by SFC to give two stereoisomers. SFC purification method: ChiralPak AS-H column (250×21.2 mm, 5 m particle size), with a mobile phase of 15-45% MeOH (+20 mM NH₃)⁺% CO₂, a variable flow rate, an injection volume of 250 μL, and a pressure of 100 bar. The collected batches were labeled “Peak 1” and “Peak 2”. Peak 1: 6-(2,6-dimethylphenyl)-2,2-dioxo-10-[4-[1-(trifluoromethyl)cyclopropyl]phenyl]-9-oxa-2λ⁶-thia-3,5,12,19-tetrazatricyclo[12.3.1.14,8]nonadeca-1(18),4(19),5,7,14,16-hexaen-13-one (24.1 mg, 12%); ¹H NMR (400 MHz, DMSO-d₆) δ 8.66 (s, 1H), 7.96 (s, 1H), 7.79-7.62 (m, 5H), 7.58 (d, J 8.2 Hz, 2H), 7.25 (t, J 7.1 Hz, 1H), 7.11 (d, J 7.7 Hz, 2H), 6.39-6.18 (m, 2H), 3.59-3.37 (m, 2H), 2.04 (s, 6H), 1.39 (d, J 2.7 Hz, 2H), 1.23 (d, J 4.0 Hz, 2H). ESI-MS m/z calc. 608.17053, found 609.1 (M+1)⁺; Retention time: 1.79 minutes; LC method A. Peak 2: 6-(2,6-dimethylphenyl)-2,2-dioxo-10-[4-[1-(trifluoromethyl)cyclopropyl]phenyl]-9-oxa-2λ⁶-thia-3,5,12,19-tetrazatricyclo[12.3.1.14,8]nonadeca-1(18),4(19),5,7,14,16-hexaen-13-one (24.0 mg, 12%); ¹H NMR (400 MHz, DMSO-d₆) δ 8.67 (s, 1H), 8.02-7.90 (m, 1H), 7.80-7.62 (m, 5H), 7.59 (d, J=8.2 Hz, 2H), 7.31-7.19 (m, 1H), 7.12 (d, J 7.2 Hz, 2H), 6.27 (dd, J 11.3, 4.7 Hz, 2H), 3.62-3.45 (m, 2H), 2.05 (s, 6H), 1.40 (d, J 2.5 Hz, 2H), 1.24 (s, 2H). ESI-MS m z calc. 608.17053, found 609.1 (M+1)⁺; Retention time: 1.79 minutes; LC method A.

### Example 20: Preparation of Compound 11

### Step 1: 2-Chloro-1-[4-[1-(trifluoromethyl)cyclopropyl]phenyl]ethanone

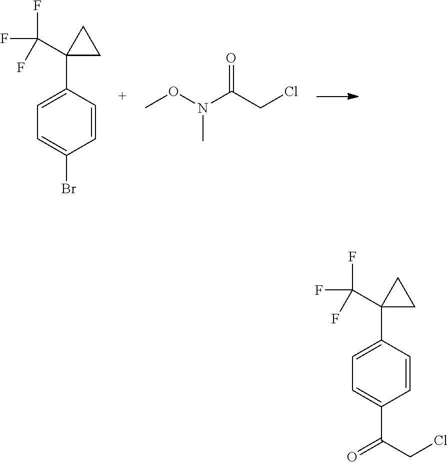

Grignard reagent formation: A 1 L three-necked round-bottomed flask was fitted with a magnetic stir bar, a reflux condenser atop the side neck, an addition funnel atop the middle neck, and a septum on the side neck. The septum on the side neck was removed temporarily to introduce chunks of Mg (25.31 g, 1.041 mol). This entire system was placed under high vacuum, and the Mg chunks were stirred vigorously without solvent to induce chipping of the metal surface. The system was flushed with nitrogen gas. A water bath was placed under the reaction flask, and anhydrous diethylether (200 mL) was added in one portion. Then, 1,2-dibromoethane (3 mL, 34.81 mmol) was introduced as an activating agent. The resulting mixture was stirred vigorously at room temperature for 10 min. Then, a solution of 1-bromo-4-[1-(trifluoromethyl)cyclopropyl]benzene (70 g, 264.1 mmol) in diethylether (65 mL) was added in five separate portions over a 90-min period. During this time, the mixture became exothermic enough to induce gentle reflux of the ether solvent. After the aryl bromide was completely added to the flask, the mixture was allowed to stir at room temperature for 15 min. When using this Grignard reagent, only the solution was taken up by a cannula, and the excess Mg chunks were left behind in the flask.

Reaction of Weinreb amide substrate: In a 500-mL round-bottomed flask, 2-chloro-N-methoxy-N-methyl-acetamide (36.2 g, 263.1 mmol) was mixed with THE (300 mL), and cooled to 0° C. Then the Grignard reagent prepared above was cannulated slowly into this mixture, and this mixture was stirred at 0° C. for 30 minutes and then warmed to room temperature while stirring over 1 h. It was then quenched by pouring onto HCl (400 mL of 2 M, 800.0 mmol), and the resulting mixture was extracted with ethyl acetate (300 mL). The combined organic extracts was washed with saturated aqueous sodium chloride solution (200 mL), then dried over sodium sulfate, filtered, and evaporated in vacuo. The resulting yellow solid was purified by silica plug using a gradient eluent of 0 to 5% ethyl acetate in hexanes to give a colorless oil, 2-chloro-1-[4-[1-(trifluoromethyl)cyclopropyl]phenyl]ethanone (31 g, 45%) ESI-MS m/z calc. 262.03723, found 263.1 (M+1)⁺; Retention time: 1.75 minutes; LC method A.

### Step 2: 2-Amino-1-{4-[1-(trifluoromethyl)cyclopropyl]phenyl}ethan-1-ol

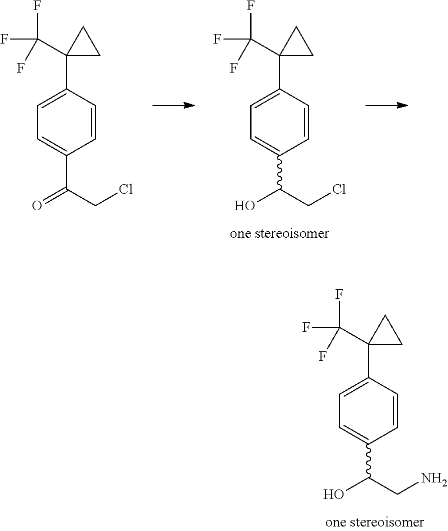 [SMILES: O=C(CCl)c1ccc(C2(C(F)(F)F)CC2)cc1 ; OC(CCl)c1ccc(C2(C(F)(F)F)CC2)cc1 ; NCC(O)c1ccc(C2(C(F)(F)F)CC2)cc1]

Stage 1: A 1 L-three neck round bottom flask was charged with THE (120 mL) and (3aR)-1-methyl-3,3-diphenyl-3a,4,5,6-tetrahydropyrrolo[1,2-c][1,3,2]oxazaborole (2.0191 g, 7.285 mmol) and the system was cooled to 5° C. under nitrogen flow. BH₃-tetrahydrofuran (176 mL of 1 M, 176.00 mmol) was slowly added to the reaction mixture via addition funnel. After the addition was complete, the reaction was warmed to 20° C. and stirred for 20 min, then cooled to 10° C. A THE (120 mL) solution of 2-chloro-1-[4-[1-(trifluoromethyl)cyclopropyl]phenyl]ethanone (30.9 g, 117.6 mmol) was slowly added to the reaction mixture over 90 min (exotherm ˜8° C.) while maintaining the reaction below 25° C. Upon completion of the ketone addition, the reaction was warmed to ambient temperature and stirred for 1 h. The reaction was cooled to 0° C. and quenched slowly with MeOH (50 mL, 1.234 mol) over 30 min and then slowly warmed to room temperature and stirred for 15 min. The reaction mixture was concentrated in vacuo and then taken up in 400 mL of ethyl acetate and poured into aqueous HCl (150 mL of 1 M, 150.0 mmol). The organic layer was separated and washed with water (100 mL) and then brine (60 mL). The organic layer was dried over anhydrous sodium sulfate, concentrated in vacuo and then dissolved in THE (100 mL) and concentrated in vacuo. The resulting light-yellow oil was not further purified. Crude weight: 33.45 g.

Stage 2: The Stage 1 product was dissolved in THE (120 mL) and cooled to 5° C. under nitrogen and then treated with NaHMDS (350 mL of 1 M, 350.0 mmol) dropwise over 1 h. Upon completion of the addition, the reaction mixture was warmed to ambient temperature and stirred for 16 h. It was then cooled to 0° C., charged with water (230 mL, 12.75 mol), and then warmed to room temperature while stirring over 3 h. The mixture was diluted with ethyl acetate (300 mL) and the organic layer was separated. The product was extracted with ethyl acetate (200 mL) and the combined organic layers were dried over anhydrous sodium sulfate, filtered, and concentrated in vacuo. The resulting residue was taken up in dioxane (40 mL) and cooled to 10-15° C. and slowly treated with HCl in dioxane (50 mL of 4 M, 200.0 mmol) to make the HCl salt. The mixture was concentrated in vacuo, upon which the product precipitated. Trituration with diethyl ether (70 mL), then filtration and drying under vacuum gave (2-amino-1-{4-[1-(trifluoromethyl)cyclopropyl]phenyl}ethan-1-ol (hydrochloride salt) (28.34 g, 86%) ESI-MS m/z calc. 245.10275, found 246.2 (M+1)⁺; Retention time: 0.97 minutes; LC method D.

### Step 3: 6-(2,6-Dimethylphenyl)-2,2-dioxo-10-[4-[1-(trifluoromethyl)cyclopropyl]phenyl]-9-oxa-2λ⁶-thia-3,5,12,19-tetrazatricyclo[12.3.1.14,8]nonadeca-1(18),4(19),5,7,14,16-hexaen-13-one (Compound 11)

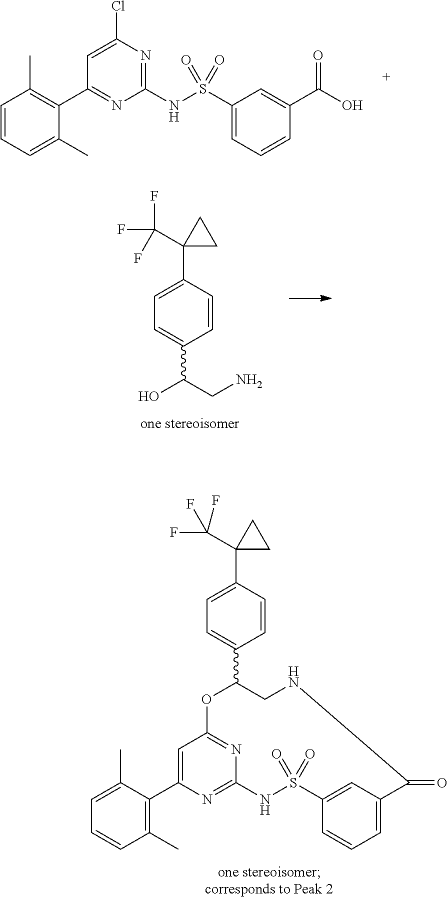 [SMILES: Cc1cccc(C)c1-c1cc(Cl)nc(NS(=O)(=O)c2cccc(C(=O)O)c2)n1 ; NCC(O)c1ccc(C2(C(F)(F)F)CC2)cc1 ; Cc1cccc(C)c1-c1cc2nc(n1)NS(=O)(=O)c1cccc(c1)C(=O)NCC(c1ccc(C3(C(F)(F)F)CC3)cc1)O2]

In a 2 L three-neck round bottom flask was charged 3-[[4-chloro-6-(2,6-dimethylphenyl)pyrimidin-2-yl]sulfamoyl]benzoic acid (33.63 g, 80.48 mmol), 2-amino-1-{4-[1-(trifluoromethyl)cyclopropyl]phenyl}ethan-1-ol (hydrochloride salt) (28.24 g, 100.2 mmol), and THE (400 mL). The suspension was stirred under a nitrogen flow and cooled to 5° C. Solid sodium t-butoxide (50.47 g, 525.2 mmol) was added in three portions and the reaction was then warmed to room while stirring over 2 h (the internal temperature was maintained below 25° C.). In a separate 2-L round-bottomed flask, a solution of HATU (61.70 g, 162.3 mmol) in DMF (800 mL) was prepared. Then, the substrate mixture in the 2-L flask was added dropwise into this HATU-filled 2-L flask over 15 min. The resulting reaction mixture was stirred at room temperature for 30 min. The reaction was cooled to 0° C. and quenched carefully with HCl (700 mL of 1 M, 700.0 mmol) and diluted with ethyl acetate (900 mL). The phases were separated, the organic layer was kept aside, and the aqueous phase was extracted with ethyl acetate (900 mL). The organic layers were combined and washed with brine (150 mL×7), then dried over anhydrous sodium sulfate, filtered, and concentrated in vacuo. The crude residue was taken up in DCM (100 mL), upon which a white precipitate crashed out. This solid was filtered (not product) and discarded; the DCM filtrate was purified by a silica plug (70% EtOAc/hexanes). Additional purification by silica gel column chromatography, followed by trituration with ethanol, gave 6-(2,6-dimethylphenyl)-2,2-dioxo-10-[4-[1-(trifluoromethyl)cyclopropyl]phenyl]-9-oxa-2λ⁶-thia-3,5,12,19-tetrazatricyclo[12.3.1.14,8]nonadeca-1(18),4(19),5,7,14,16-hexaen-13-one (13.005 g, 26%). ¹H NMR (500 MHz, DMSO-d₆) δ 13.26-11.96 (broad d, 2H), 8.66 (s, 1H), 7.96 (s, 1H), 7.74 (dd, J 8.6, 5.7 Hz, 1H), 7.69 (s, 1H), 7.65 (d, J 8.3 Hz, 2H), 7.59 (d, J 8.2 Hz, 2H), 7.25 (t, J 7.1 Hz, 1H), 7.11 (d, J 7.7 Hz, 2H), 6.31 (s, 1H), 6.26 (dd, J 10.7, 3.7 Hz, 1H), 3.53 (ddd, J 13.8, 9.3, 4.1 Hz, 1H), 3.40-3.31 (m, 1H), 2.04 (s, 6H), 1.43-1.36 (m, 2H), 1.15 (q, J 10.2 Hz, 2H). ESI-MS m/z calc. 608.17053, found 609.0 (M+1)⁺; Retention time: 1.84 minutes; LC method A.

### Example 21: Preparation of Compound 12

### Step 1: 1-(4-Cyclobutylphenyl)-2-nitro-ethanol, and 2-amino-1-(4-cyclobutylphenyl)ethanol

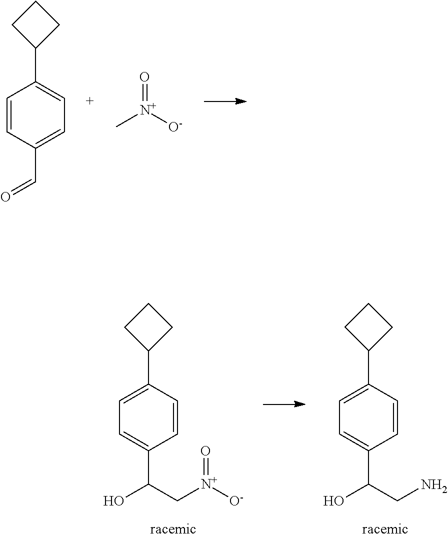

Stage 1: A solution of 4-cyclobutylbenzaldehyde (1.0 g, 6.242 mmol) and nitromethane (700 μL, 12.92 mmol) in THF (16 mL) was treated with THE solution of sodium methoxide (1.45 mL of 25% w/v, 6.710 mmol) at 0° C. and stirred at RT for 3 h. The mixture was quenched with ammonium chloride and extracted with DCM. The organic extract was dried over sodium sulfate, filtered and evaporated in vacuo to afford crude 1-(4-cyclobutylphenyl)-2-nitro-ethanol (1.3 g, 94%). ¹H NMR (400 MHz, Chloroform-d) δ 7.34-7.28 (m, 2H), 7.25 (d, J 8.2 Hz, 2H), 5.43 (dd, J 9.7, 3.0 Hz, 1H), 4.60 (dd, J 13.3, 9.7 Hz, 1H), 4.49 (dd, J 13.3, 3.0 Hz, 1H), 3.55 (p, J 8.5 Hz, 1H), 2.78 (s, 1H), 2.35 (qt, J 7.8, 2.4 Hz, 2H), 2.18-2.00 (m, 3H), 1.89-1.82 (m, 1H). ESI-MS m/z calc. 221.1052, Retention time: 1.44 minutes; LC method A.

Stage 2: A solution of the nitroalkane (from Stage 1) in MeOH (35 mL) was purged with nitrogen for 10 min. Pd/C (150 mg of 10% w/w, 0.1410 mmol) was added, and then the reaction mixture was placed under a balloon of hydrogen with steady purging as the mixture was stirred at RT for 16 h. Then, the mixture was purged with nitrogen for 5 minutes, filtered and evaporated in vacuo to afford a white solid, 2-amino-1-(4-cyclobutylphenyl)ethanol (1.0 g, 84%). ¹H NMR (400 MHz, Chloroform-d) δ 7.33-7.25 (m, 2H), 7.20 (d, J 8.0 Hz, 2H), 4.72-4.54 (m, 1H), 3.53 (t, J 8.8 Hz, 1H), 2.99 (dd, J 12.7, 3.8 Hz, 1H), 2.82 (dd, J 12.7, 7.7 Hz, 1H), 2.33 (dddt, J 10.3, 7.8, 4.6, 2.4 Hz, 5H), 2.19-2.08 (m, 2H), 2.07-1.95 (m, 1H), 1.89-1.80 (m, 1H). ESI-MS m/z calc. 191.13101, found 192.2 (M+1)⁺; Retention time: 0.82 minutes; LC method A.

### Step 2: 10-(4-Cyclobutylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 12)

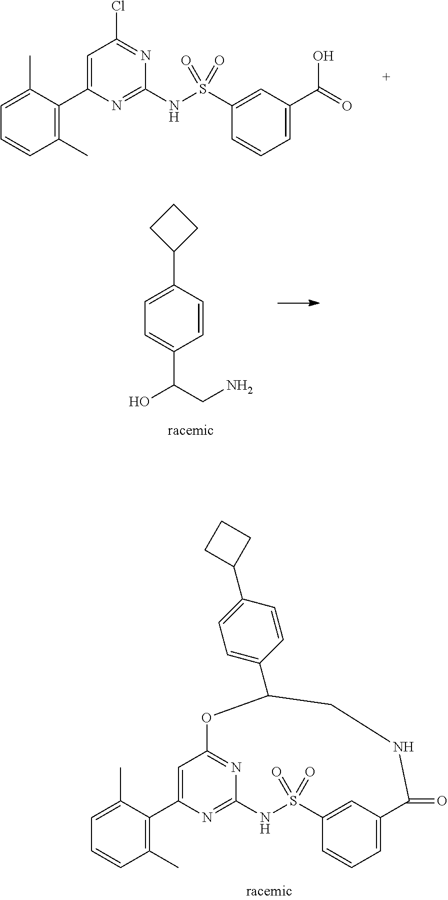 [SMILES: Cc1cccc(C)c1-c1cc(Cl)nc(NS(=O)(=O)c2cccc(C(=O)O)c2)n1 ; NCC(O)c1ccc(C2CCC2)cc1 ; Cc1cccc(C)c1-c1cc2nc(n1)NS(=O)(=O)c1cccc(c1)C(=O)NCC(c1ccc(C3CCC3)cc1)O2]

Stage 1: In a 250 mL flask, 3-[[4-chloro-6-(2,6-dimethylphenyl)pyrimidin-2-yl]sulfamoyl]benzoic acid (2.23 g, 5.225 mmol), 2-amino-1-(4-cyclobutylphenyl)ethanol (1.0 g, 5.228 mmol) and THE (48 mL) were mixed and cooled in an ice bath at 0° C., to which KOtBu (2.48 g, 22.10 mmol) was added. This mixture was stirred for 2 h at 0° C. This intermediate material was not isolated but carried on to the next stage in one pot.

Stage 2: The material in the same pot at 0° C. was diluted with DMF (25 mL), to which HATU (3.0 g, 7.890 mmol) and then DIPEA (3.1 mL, 17.80 mmol) were added. After stirring at room temperature for 30 min, the cyclization did not occur, so it was quenched by addition of 10% aqueous citric acid (60 mL). The mixture was extracted with ethyl acetate (2×60 mL). The combined organic phase was washed with water (2×50 mL), then brine (50 mL), dried (over sodium sulfate), filtered and concentrated. This crude product was purified by silica gel chromatography (120 gram column) using a gradient from 100% hexanes to 100% ethyl acetate to give 3-[[4-[2-amino-1-(4-cyclobutylphenyl)ethoxy]-6-(2,6-dimethylphenyl)pyrimidin-2-yl]sulfamoyl]benzoic acid (2.7 g, 90%) ESI-MS m/z calc. 572.20935, found 573.2 (M+1)⁺; Retention time: 1.33 minutes; LC method A.

Stage 3: The product from Stage 2 was cooled to 0° C. and diluted with DMF (25 mL), to which HATU (3.0 g, 7.890 mmol) and DIPEA (3.1 mL, 17.80 mmol) were added. After stirring at room temperature for 30 min, it was quenched with 10% aqueous citric acid (60 mL). The product precipitated out and the white solid was collected by vacuum. The collected solid material was re-dissolved with ethyl acetate (120 mL). The organic solution was washed with water (2×50 mL), then brine (50 mL), dried (over sodium sulfate), filtered and concentrated. This crude product was purified by silica gel chromatography (80 gram column) using a gradient from 100% dichloromethane to 20% methanol in dichloromethane followed by reverse-phase preparative chromatography utilizing a C₁₈ column to afford a pure white solid, 10-(4-cyclobutylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (19.78 mg, 1%). ¹H NMR (400 MHz, DMSO-d₆) δ 13.04 (s, 1H), 8.65 (s, 1H), 7.96 (s, 1H), 7.69 (dd, J 9.3, 5.2 Hz, 3H), 7.54 (d, J 8.1 Hz, 2H), 7.36 (d, J 7.9 Hz, 2H), 7.25 (t, J 7.7 Hz, 1H), 7.12 (d, J 7.6 Hz, 2H), 6.31 (s, 1H), 6.23 (d, J=10.8 Hz, 1H), 3.63-3.47 (m, 2H), 3.37 (d, J=5.9 Hz, 1H), 2.33 (qt, J=8.0, 2.5 Hz, 2H), 2.19-2.11 (m, 2H), 2.11-2.01 (m, 6H), 2.01-1.94 (m, 1H), 1.89-1.79 (m, 1H). ESI-MS m/z calc. 554.1988, found 555.3 (M+1)⁺; Retention time: 1.81 minutes; LC method A.

### Example 22: Preparation of Compound 13 and Compound 14

### Step 1: 10-(4-Cyclobutylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, Peak 1 (Compound 13), and 10-(4-cyclobutylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, Peak 2 (Compound 14)

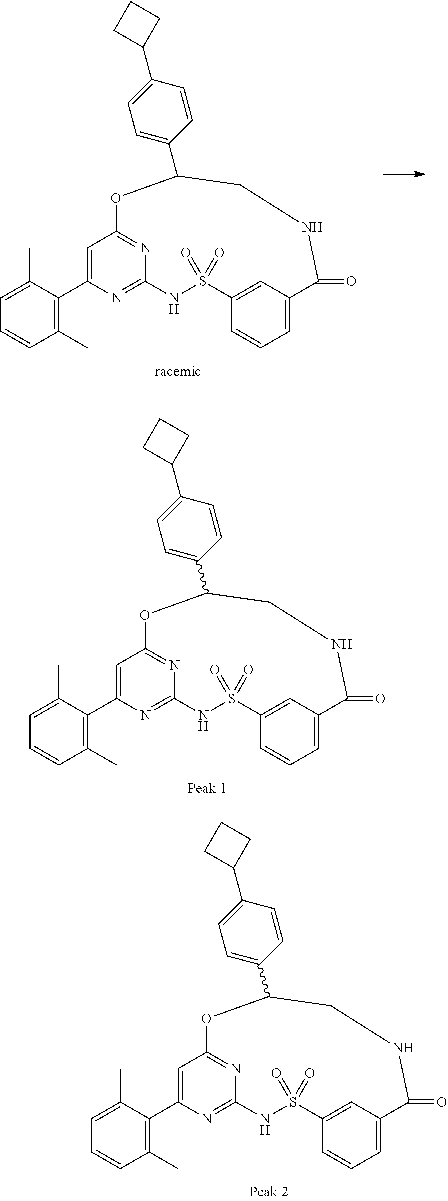 [SMILES: Cc1cccc(C)c1-c1cc2nc(n1)NS(=O)(=O)c1cccc(c1)C(=O)NCC(c1ccc(C3CCC3)cc1)O2 ; Cc1cccc(C)c1-c1cc2nc(n1)NS(=O)(=O)c1cccc(c1)C(=O)NCC(c1ccc(C3CCC3)cc1)O2 ; Cc1cccc(C)c1-c1cc2nc(n1)NS(=O)(=O)c1cccc(c1)C(=O)NCC(c1ccc(C3CCC3)cc1)O2]

Racemic 10-(4-cyclobutylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (19 mg, 0.03408 mmol) was separated into the individual enantiomers using chiral SFC (AS-3 column). SFC Peak 1 was further purified by reverse-phase preparative chromatography utilizing a Cis column to afford a white solid, 10-(4-cyclobutylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (9.3 mg, 98%) ESI-MS m/z calc. 554.1988, found 555.3 (M+1)⁺; Retention time: 1.81 minutes; LC method A. ¹H NMR (400 MHz, DMSO-d₆) δ 13.01 (s, 1H), 8.65 (s, 1H), 7.95 (s, 1H), 7.68 (d, J 6.2 Hz, 3H), 7.58-7.45 (m, 2H), 7.36 (d, J 8.1 Hz, 2H), 7.24 (t, J 7.6 Hz, 1H), 7.11 (d, J 7.6 Hz, 2H), 6.29 (s, 1H), 6.22 (dd, J 10.8, 4.1 Hz, 1H), 3.63-3.47 (m, 2H), 3.37 (d, J 5.3 Hz, 1H), 2.33 (qt, J 8.0, 2.4 Hz, 2H), 2.15 (ddt, J 14.9, 9.1, 2.8 Hz, 2H), 2.10-2.01 (m, 6H), 1.98 (td, J 9.9, 9.0, 7.2 Hz, 1H), 1.89-1.79 (m, 1H). SFC Peak 2 was further purified by reverse-phase preparative chromatography utilizing a C₁₈ column to afford a white solid, 10-(4-cyclobutylphenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (8.7 mg, 92%) ESI-MS m/z calc. 554.1988, found 555.3 (M+1)⁺; Retention time: 1.81 minutes; LC method A. ¹H NMR (400 MHz, DMSO-d₆) δ 12.90 (s, 1H), 8.65 (s, 1H), 7.94 (d, J 6.4 Hz, 1H), 7.67 (d, J 6.2 Hz, 3H), 7.53 (d, J 7.9 Hz, 2H), 7.36 (d, J 7.9 Hz, 2H), 7.23 (d, J 7.8 Hz, 1H), 7.10 (d, J 7.6 Hz, 2H), 6.24 (s, 1H), 6.23-6.14 (m, 1H), 3.60-3.48 (m, 2H), 3.36 (d, J 13.7 Hz, 1H), 2.33 (qt, J 7.9, 2.5 Hz, 2H), 2.15 (ddt, J 11.8, 8.9, 2.9 Hz, 2H), 2.04 (s, 6H), 2.01-1.96 (m, 1H), 1.89-1.80 (m, 1H).

### Example 23: Preparation of Compound 15

### Step 1: tert-Butyl N-[2-oxo-2-(4-trimethylsilylphenyl)ethyl]carbamate

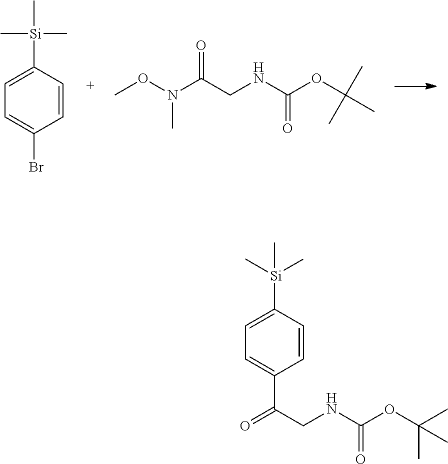

To a solution of (4-bromophenyl)-trimethyl-silane (4.2 mL, 21.50 mmol) in THE (100 mL) was treated with chloro(isopropyl)magnesium; chlorolithium (17 mL of 1.3 M, 22.10 mmol) at RT. After addition, the mixture was stirred at room temperature for 10 min then heated to 40° C. for 2 h. It was cooled to room temperature, then a solution of tert-butyl N-[2-[methoxy(methyl)amino]-2-oxo-ethyl]carbamate (2.25 g, 10.31 mmol) in THE (50 mL) was added dropwise at RT. The mixture was stirred at RT for 16 h and quenched with ammonium chloride, extracted with EtOAc and organic phase was dried over sodium sulfate and evaporated. The residue was purified by silica gel chromatography using (120 g silica, 100% hexanes to 75% hexanes in ethyl acetate) to give a colorless oil, tert-butyl N-[2-oxo-2-(4-trimethylsilylphenyl)ethyl]carbamate (404 mg, 13%). ¹H NMR (500 MHz, DMSO-d₆) δ 7.93 (d, J 7.8 Hz, 2H), 7.67 (d, J 7.7 Hz, 2H), 7.06 (t, J 5.9 Hz, 1H), 4.44 (d, J 5.9 Hz, 2H), 1.39 (s, 9H), 0.27 (s, 9H). ESI-MS m/z calc. 307.16037, found 308.2 (M+1)⁺; Retention time: 1.95 minutes; LC method A.

### Step 2: tert-Butyl N-[2-hydroxy-2-(4-trimethylsilylphenyl)ethyl]carbamate

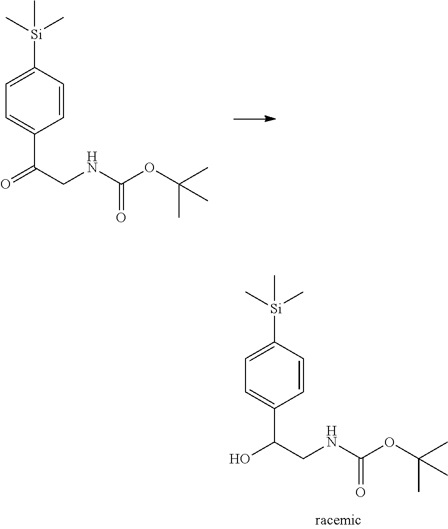 [SMILES: CC(C)(C)OC(=O)NCC(=O)c1ccc([Si](C)(C)C)cc1 ; CC(C)(C)OC(=O)NCC(O)c1ccc([Si](C)(C)C)cc1]

tert-Butyl N-[2-oxo-2-(4-trimethylsilylphenyl)ethyl]carbamate (400 mg, 1.301 mmol) was dissolved in MeOH (5 mL) and treated with sodium borohydride (30 mg, 0.7930 mmol) at 0° C. and stirred at RT for 1 h. The mixture was evaporated, quenched with aqueous sodium bicarbonate and extracted with EtOAc. The organic phase was dried over sodium sulfate, evaporated and purified by silica gel chromatography (24 g silica, 100% hexanes to 75% hexanes in ethyl acetate) to give a white solid, tert-butyl N-[2-hydroxy-2-(4-trimethylsilylphenyl)ethyl]carbamate (390 mg, 97%). ¹H NMR (500 MHz, DMSO-d₆) δ 7.46 (d, J 7.6 Hz, 2H), 7.29 (d, J 7.6 Hz, 2H), 6.72 (t, J 5.9 Hz, 1H), 5.33 (d, J 4.5 Hz, 1H), 4.56 (dt, J 9.2, 5.0 Hz, 1H), 3.15-3.06 (m, 1H), 3.00 (ddd, J 13.3, 7.8, 5.3 Hz, 1H), 1.39-1.30 (m, 9H), 0.23-0.21 (m, 9H). ESI-MS m/z calc. 309.17603, found 310.2 (M+1)⁺; Retention time: 1.81 minutes; LC method A.

### Step 3: 2-Amino-1-(4-trimethylsilylphenyl)ethanol

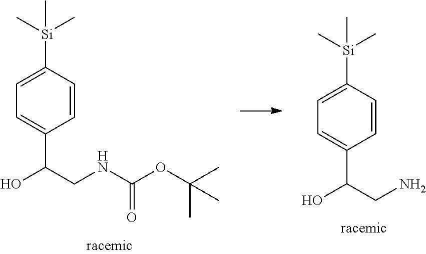 [SMILES: CC(C)(C)OC(=O)NCC(O)c1ccc([Si](C)(C)C)cc1 ; C[Si](C)(C)c1ccc(C(O)CN)cc1]

In a 100 mL flask, tert-butyl N-[2-hydroxy-2-(4-trimethylsilylphenyl)ethyl]carbamate (385 mg, 1.244 mmol) was dissolved in DCM (5.0 mL) and treated with dioxane solution of HCl (6 mL of 4 M, 24.00 mmol) and stirred at RT for 16 h. The mixture was evaporated in vacuo to give a white solid, 2-amino-1-(4-trimethylsilylphenyl)ethanol (hydrochloride salt) (300 mg, 98%). ESI-MS m/z calc. 209.1236, found 210.2 (M+1)⁺; Retention time: 0.99 minutes; LC method A.

### Step 4: 3-[[4-[2-(tert-Butoxycarbonylamino)-1-(4-trimethylsilylphenyl)ethoxy]-6-(2,6-dimethylphenyl)pyrimidin-2-yl]sulfamoyl]benzoic Acid

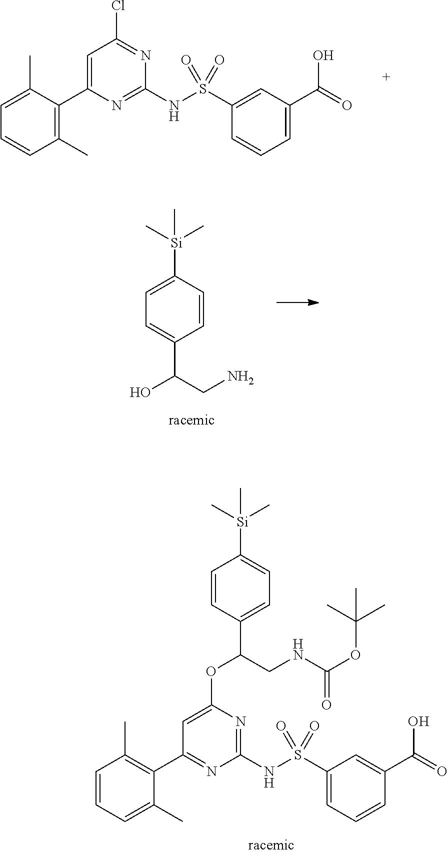 [SMILES: Cc1cccc(C)c1-c1cc(Cl)nc(NS(=O)(=O)c2cccc(C(=O)O)c2)n1 ; C[Si](C)(C)c1ccc(C(O)CN)cc1 ; Cc1cccc(C)c1-c1cc(OC(CNC(=O)OC(C)(C)C)c2ccc([Si](C)(C)C)cc2)nc(NS(=O)(=O)c2cccc(C(=O)O)c2)n1]

In a 100 mL flask, 3-[[4-chloro-6-(2,6-dimethylphenyl)pyrimidin-2-yl]sulfamoyl]benzoic acid (353 mg, 0.8448 mmol), 2-amino-1-(4-trimethylsilylphenyl)ethanol (hydrochloride salt) (208 mg, 0.8461 mmol) and THE (8.115 mL) were mixed and cooled in an ice bath at 0° C., to which KOtBu (610 mg, 5.436 mmol) was added. This mixture was stirred 2 h at RT. Then, the mixture was re-cooled to 0° C. and Boc anhydride (300 mg, 1.375 mmol) was added, allowing to stir for 16 h while warming to RT. The mixture was then diluted with ethyl acetate and quenched with saturated ammonium chloride solution and then extracted with ethyl acetate (3×150 mL). The combined organic extract was washed with water (150 mL) and saturated brine solution (150 mL), then dried over sodium sulfate, filtered, and evaporated in vacuo. This crude product was purified on silica gel chromatography (80 gram silica using a gradient from 100% dichloromethane to 10% methanol in dichloromethane) to afford a light yellow solid, 3-[[4-[2-(tert-butoxycarbonylamino)-1-(4-trimethylsilylphenyl)ethoxy]-6-(2,6-dimethylphenyl)pyrimidin-2-yl]sulfamoyl]benzoic acid (548 mg, 94%) ESI-MS m/z calc. 690.25433, found 691.2 (M+1)⁺; Retention time: 2.11 minutes; LC method A.

### Step 5: 3-[[4-[2-Amino-1-(4-trimethylsilylphenyl)ethoxy]-6-(2,6-dimethylphenyl)pyrimidin-2-yl]sulfamoyl]benzoic Acid

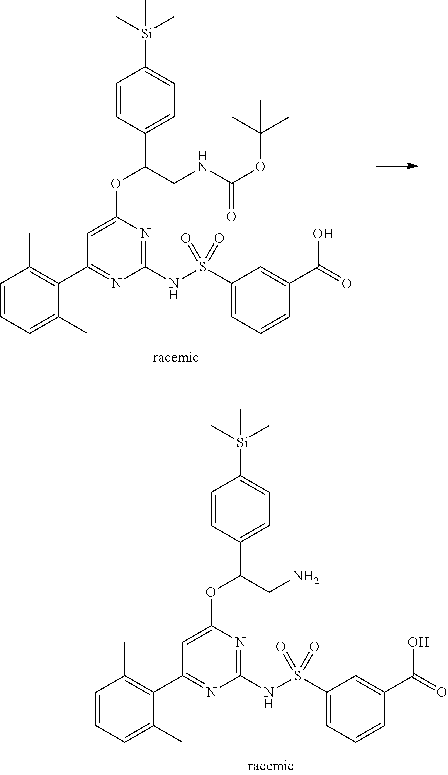 [SMILES: Cc1cccc(C)c1-c1cc(OC(CNC(=O)OC(C)(C)C)c2ccc([Si](C)(C)C)cc2)nc(NS(=O)(=O)c2cccc(C(=O)O)c2)n1 ; Cc1cccc(C)c1-c1cc(OC(CN)c2ccc([Si](C)(C)C)cc2)nc(NS(=O)(=O)c2cccc(C(=O)O)c2)n1]

3-[[4-[2-(tert-Butoxycarbonylamino)-1-(4-trimethylsilylphenyl)ethoxy]-6-(2,6-dimethylphenyl)pyrimidin-2-yl]sulfamoyl]benzoic acid (548 mg, 0.7932 mmol) was dissolved in DCM (13.0 mL) and HCl in dioxane (2.5 mL of 4 M, 10.00 mmol) was added. This mixture was stirred at room temperature for 1 h. The reaction mixture was concentrated under reduced pressure to a yellow solid, which was then slurried in diethyl ether (2×20 mL). The solid was collected by vacuum filtration to provide as a light yellow solid 3-[[4-[2-amino-1-(4-trimethylsilylphenyl)ethoxy]-6-(2,6-dimethylphenyl)pyrimidin-2-yl]sulfamoyl]benzoic Acid (hydrochloride salt) (475 mg, 95%) ESI-MS m/z calc. 590.2019, found 591.2 (M+1)⁺; Retention time: 1.41 minutes; LC method A.

### Step 6: 6-(2,6-Dimethylphenyl)-2,2-dioxo-10-(4-trimethylsilylphenyl)-9-oxa-2λ⁶-thia-3,5,12,19-tetrazatricyclo[12.3.1.14,8]nonadeca-1(18),4(19),5,7,14,16-hexaen-13-one (Compound 15)

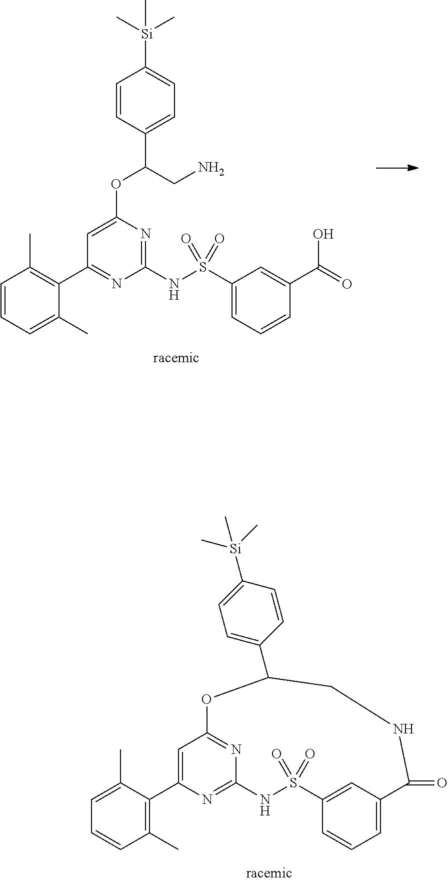 [SMILES: Cc1cccc(C)c1-c1cc(OC(CN)c2ccc([Si](C)(C)C)cc2)nc(NS(=O)(=O)c2cccc(C(=O)O)c2)n1 ; Cc1cccc(C)c1-c1cc2nc(n1)NS(=O)(=O)c1cccc(c1)C(=O)NCC(c1ccc([Si](C)(C)C)cc1)O2]

In a 100-mL flask, 3-[[4-[2-amino-1-(4-trimethylsilylphenyl)ethoxy]-6-(2,6-dimethylphenyl)pyrimidin-2-yl]sulfamoyl]benzoic acid (hydrochloride salt) (415 mg, 0.6616 mmol) was dissolved in DMF (12 mL), to which HATU (400 mg, 1.052 mmol) and then DIPEA (1 mL, 5.741 mmol) were added. After stirring at room temperature for 30 min, the mixture was quenched with water. The mixture was extracted with ethyl acetate (3×75 mL). The combined organic extract was washed with water (50 mL) and saturated brine solution (50 mL), then dried over sodium sulfate, filtered, and evaporated in vacuo. This crude product was purified by silica gel chromatography using a 40 g column eluting with 100% dichloromethane to 15% methanol in dichloromethane to afford a light yellow solid, which was further purified by reverse-phase preparative chromatography utilizing a C₁₈ column to give a white solid, 6-(2,6-dimethylphenyl)-2,2-dioxo-10-(4-trimethylsilylphenyl)-9-oxa-2λ⁶-thia-3,5,12,19-tetrazatricyclo[12.3.1.14,8]nonadeca-1(18),4(19),5,7,14,16-hexaen-13-one (86.44 mg, 23%). ¹H NMR (500 MHz, DMSO-d₆) δ 12.96 (s, 1H), 8.68 (s, 1H), 7.97 (d, J 6.0 Hz, 1H), 7.73-7.68 (m, 2H), 7.65 (d, J 7.8 Hz, 2H), 7.60 (d, J 7.7 Hz, 2H), 7.24 (t, J 7.7 Hz, 1H), 7.11 (d, J 7.7 Hz, 2H), 6.31 (s, 1H), 6.24 (dd, J=10.9, 4.2 Hz, 1H), 4.13 (s, 2H), 3.60-3.49 (m, 1H), 2.05 (d, J 12.3 Hz, 6H), 0.28 (s, 9H). ESI-MS m/z calc. 572.19135, found 573.2 (M+1)⁺; Retention time: 1.9 minutes; LC method A.

### Example 24: Preparation of Compound 16 and Compound 17

### Step 1: 6-(2,6-Dimethylphenyl)-2,2-dioxo-10-(4-trimethylsilylphenyl)-9-oxa-2λ⁶-thia-3,5,12,19-tetrazatricyclo[12.3.1.14,8]nonadeca-1(18),4(19),5,7,14,16-hexaen-13-one, SFC peak 1 (Compound 16), and 6-(2,6-dimethylphenyl)-2,2-dioxo-10-(4-trimethylsilylphenyl)-9-oxa-2λ⁶-thia-3,5,12,19-tetrazatricyclo[12.3.1.14,8]nonadeca-1(18),4(19),5,7,14,16-hexaen-13-one, SFC peak 2 (Compound 17)

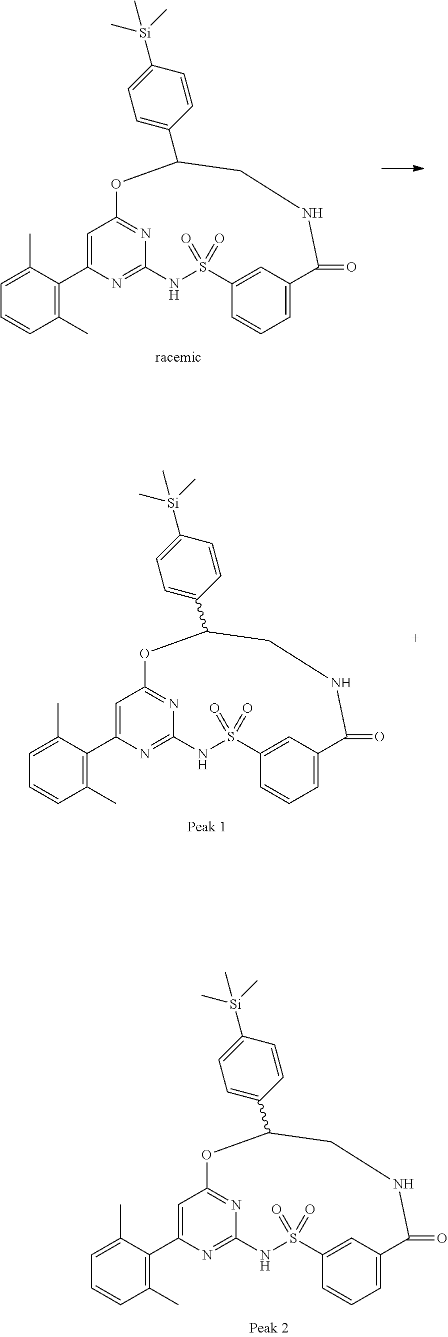 [SMILES: Cc1cccc(C)c1-c1cc2nc(n1)NS(=O)(=O)c1cccc(c1)C(=O)NCC(c1ccc([Si](C)(C)C)cc1)O2 ; Cc1cccc(C)c1-c1cc2nc(n1)NS(=O)(=O)c1cccc(c1)C(=O)NCC(c1ccc([Si](C)(C)C)cc1)O2 ; Cc1cccc(C)c1-c1cc2nc(n1)NS(=O)(=O)c1cccc(c1)C(=O)NCC(c1ccc([Si](C)(C)C)cc1)O2]

Racemic 6-(2,6-dimethylphenyl)-2,2-dioxo-10-(4-trimethylsilylphenyl)-9-oxa-2λ⁶-thia-3,5,12,19-tetrazatricyclo[12.3.1.14,8]nonadeca-1(18),4(19),5,7,14,16-hexaen-13-one (80 mg, 0.1397 mmol) was separated into the individual enantiomers using chiral SFC (AS-3 column). SFC Peak 1 was further purified by reverse-phase preparative chromatography utilizing a C₁₈ column to give a white solid: 6-(2,6-dimethylphenyl)-2,2-dioxo-10-(4-trimethylsilylphenyl)-9-oxa-2λ⁶-thia-3,5,12,19-tetrazatricyclo[12.3.1.14,8]nonadeca-1(18),4(19),5,7,14,16-hexaen-13-one (26.32 mg, 65%) ESI-MS m/z calc. 572.19135, found 573.4 (M+1)⁺; Retention time: 1.9 minutes; LC method A. SFC Peak 2 was further purified by reverse-phase preparative chromatography utilizing a C₁₈ column to give a white solid: 6-(2,6-dimethylphenyl)-2,2-dioxo-10-(4-trimethylsilylphenyl)-9-oxa-2λ⁶-thia-3,5,12,19-tetrazatricyclo[12.3.1.14,8]nonadeca-1(18),4(19),5,7,14,16-hexaen-13-one (24.51 mg, 61%)¹H NMR (500 MHz, DMSO-d₆) δ 13.00 (s, 1H), 8.67 (s, 1H), 7.97 (d, J 6.5 Hz, 1H), 7.79-7.56 (m, 7H), 7.26 (t, J 7.6 Hz, 1H), 7.12 (d, J 7.6 Hz, 2H), 6.32 (s, 1H), 6.24 (dd, J 11.4, 4.1 Hz, 1H), 3.55 (dd, J 14.6, 9.4 Hz, 1H), 3.34 (d, J 5.1 Hz, 1H), 2.05 (s, 6H), 0.29 (s, 9H). ESI-MS m/z calc. 572.19135, found 573.4 (M+1)⁺; Retention time: 1.9 minutes; LC method A.

### Example 25: Preparation of Compound 18, Compound 19, and Compound 20

### Step 1: 3-[[4-[2-Amino-1-(4-bromophenyl)ethoxy]-6-(2,6-dimethylphenyl)pyrimidin-2-yl]sulfamoyl]benzoic Acid

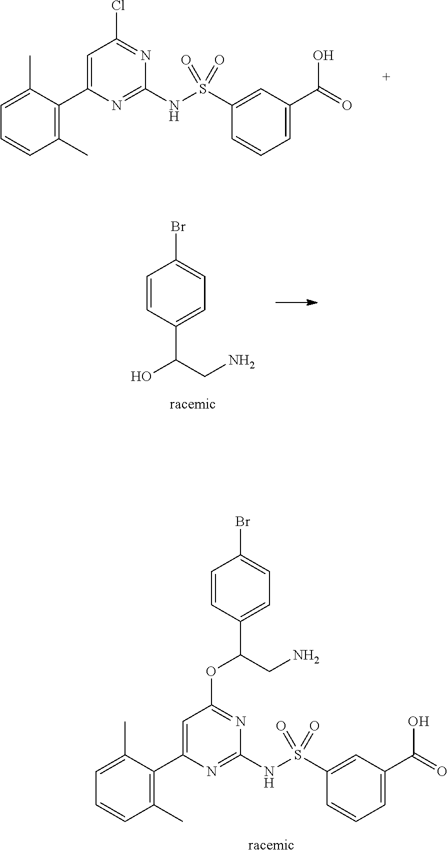 [SMILES: Cc1cccc(C)c1-c1cc(Cl)nc(NS(=O)(=O)c2cccc(C(=O)O)c2)n1 ; NCC(O)c1ccc(Br)cc1 ; Cc1cccc(C)c1-c1cc(OC(CN)c2ccc(Br)cc2)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (120 mg, 0.2872 mmol), sodium tert-butoxide (140 mg, 1.457 mmol) and 2-amino-1-(4-bromophenyl)ethanol (75 mg, 0.3471 mmol) in THE (3 mL) was stirred at RT for 1 h. The mixture was immediately quenched with a small amount of methanol and purified by preparative reverse phase HPLC (C₁₈) to afford 3-[[4-[2-amino-1-(4-bromophenyl)ethoxy]-6-(2,6-dimethylphenyl)pyrimidin-2-yl]sulfamoyl]benzoic acid (hydrochloride salt) (103.5 mg, 57%) ESI-MS m/z calc. 596.0729, found 599.1 (M+1)⁺; Retention time: 1.28 minutes; LC method A.

### Step 2: 10-(4-Bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, racemic mixture (Compound 18), and 10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, peak 1 (Compound 19), and 10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, peak 2 (Compound 20)

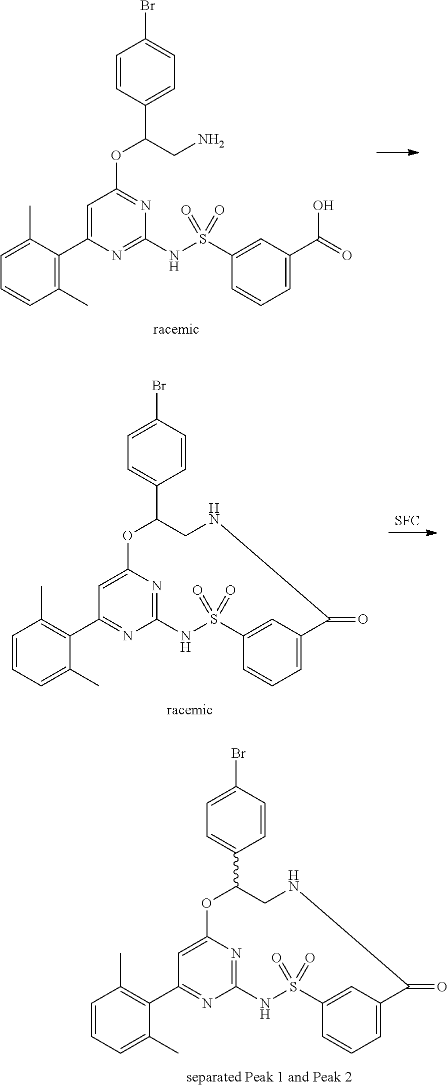 [SMILES: Cc1cccc(C)c1-c1cc(OC(CN)c2ccc(Br)cc2)nc(NS(=O)(=O)c2cccc(C(=O)O)c2)n1 ; Cc1cccc(C)c1-c1cc2nc(n1)NS(=O)(=O)c1cccc(c1)C(=O)NCC(c1ccc(Br)cc1)O2 ; "SFC" ; Cc1cccc(C)c1-c1cc2nc(n1)NS(=O)(=O)c1cccc(c1)C(=O)NCC(c1ccc(Br)cc1)O2]

A solution of 3-[[4-[2-amino-1-(4-bromophenyl)ethoxy]-6-(2,6-dimethylphenyl)pyrimidin-2-yl]sulfamoyl]benzoic acid (hydrochloride salt) (35 mg, 0.05521 mmol) and 1-diphenylphosphoryloxy-2,3,4,5,6-pentafluoro-benzene (32 mg, 0.08328 mmol) in DMF (1.5 mL) was treated with DIPEA (22 mg, 0.1702 mmol) and stirred at RT for 1 h. The mixture was filtered and purified by preparative reverse phase HPLC (C₁₈) to afford racemic 10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (16.5 mg, 52%) ESI-MS m/z calc. 578.0623, found 581.13 (M+1)⁺; Retention time: 1.68 minutes; LC method A.

This racemic mixture was purified by an SFC purification method using a ChiralPak AS-H column (250×21.2 mm, 5 m particle size), with a mobile phase of 44% of 9:1 MeCN:MeOH (no modifier)⁺56% CO₂, a flow rate of 70 mL/min, an injection volume of 400 μL, and a pressure of 100 bar. The collected batches were labeled “Peak 1” and “Peak 2”. Peak 1 (shorter retention time, first to elute): 10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.3 mg, 33%) ESI-MS m/z calc. 578.0623, found 581.17 (M+1)⁺; Retention time: 1.67 minutes; LC method A. Peak 2 (longer retention time, second to elute):10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.0 mg, 31%) ESI-MS m/z calc. 578.0623, found 581.17 (M+1)⁺; Retention time: 1.67 minutes; LC method A.

### Example 26: Preparation of Compound 18

### Step 1: 3-[[4-[1-(4-Bromophenyl)-2-(tert-butoxycarbonylamino)ethoxy]-6-(2,6-dimethylphenyl)pyrimidin-2-yl]sulfamoyl]benzoic Acid

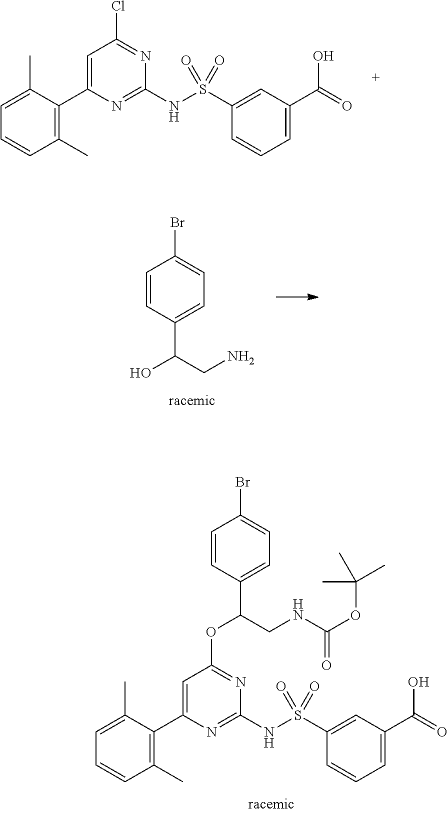 [SMILES: Cc1cccc(C)c1-c1cc(Cl)nc(NS(=O)(=O)c2cccc(C(=O)O)c2)n1 ; NCC(O)c1ccc(Br)cc1 ; Cc1cccc(C)c1-c1cc(OC(CNC(=O)OC(C)(C)C)c2ccc(Br)cc2)nc(NS(=O)(=O)c2cccc(C(=O)O)c2)n1]

To a stirring solution of 3-[[4-chloro-6-(2,6-dimethylphenyl)pyrimidin-2-yl]sulfamoyl]benzoic acid (10 g, 23.931 mmol) and 2-amino-1-(4-bromophenyl)ethanol (7.7562 g, 35.896 mmol) in anhydrous THE (250 mL) at 0° C. under nitrogen, sodium tert-butoxide (9.1994 g, 95.724 mmol) was added portion-wise. After the addition was complete, the reaction was stirred at 0° C. for 2 h, then warmed to room temperature and stirred for 1 h. Di-tert-butyl dicarbonate (8.8789 g, 40.683 mmol) was added and the stirring was continued overnight. The reaction was quenched with saturated aqueous ammonium chloride (50 mL). The volatiles were removed under vacuum and the product was extracted with ethyl acetate (3×120 mL). The combined organic layers were washed with brine (30 mL), dried over anhydrous sodium sulfate and concentrated. The crude was purified by silica gel chromatography using 0-50% ethyl acetate-hexanes, followed by 0-15% DCM-methanol to afford 3-[[4-[1-(4-bromophenyl)-2-(tert-butoxycarbonylamino)ethoxy]-6-(2,6-dimethylphenyl)pyrimidin-2-yl]sulfamoyl]benzoic acid (7.91 g, 45%) as a pale-yellow solid. ESI-MS m/z calc. 696.1253, found 697.5 (M+1)⁺; Retention time: 5.98 minutes; LC method S.

### Step 2: 3-[[4-[2-Amino-1-(4-bromophenyl)ethoxy]-6-(2,6-dimethylphenyl)pyrimidin-2-yl]sulfamoyl]benzoic Acid

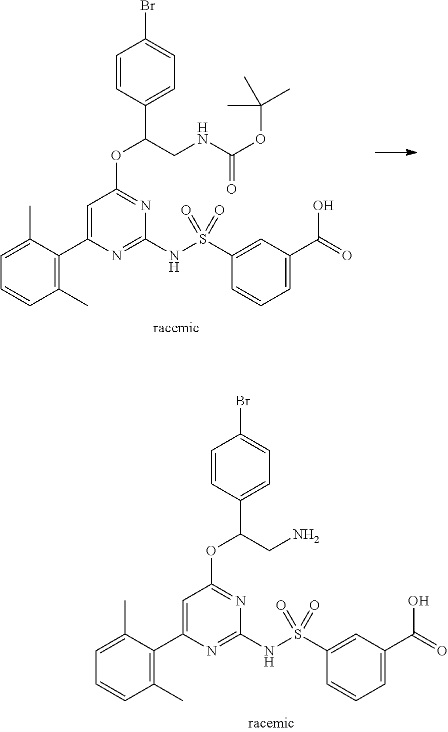 [SMILES: Cc1cccc(C)c1-c1cc(OC(CNC(=O)OC(C)(C)C)c2ccc(Br)cc2)nc(NS(=O)(=O)c2cccc(C(=O)O)c2)n1 ; Cc1cccc(C)c1-c1cc(OC(CN)c2ccc(Br)cc2)nc(NS(=O)(=O)c2cccc(C(=O)O)c2)n1]

To a stirring solution of 3-[[4-[1-(4-bromophenyl)-2-(tert-butoxycarbonylamino)ethoxy]-6-(2,6-dimethylphenyl)pyrimidin-2-yl]sulfamoyl]benzoic Acid (7.91 g, 10.772 mmol) in DCM (80 mL) at room temperature was added HCl (12 mL of 4 M solution in 1,4-dioxane, 48.000 mmol). The reaction mixture was stirred for 2 h and the product precipitated out. The volatiles were removed under vacuum to afford 3-[[4-[2-amino-1-(4-bromophenyl)ethoxy]-6-(2,6-dimethylphenyl)pyrimidin-2-yl]sulfamoyl]benzoic Acid (hydrochloride salt) (6.35 g, 87%) as a pale-yellow solid. ESI-MS m/z calc. 596.0729, found 597.5 (M+1)⁺; Retention time: 3.95 minutes; LC method S.

### Step 3: 10-(4-Bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 18)

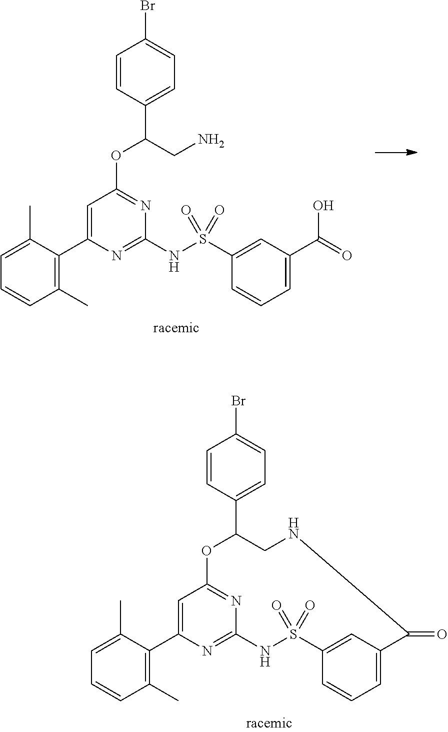 [SMILES: Cc1cccc(C)c1-c1cc(OC(CN)c2ccc(Br)cc2)nc(NS(=O)(=O)c2cccc(C(=O)O)c2)n1 ; Cc1cccc(C)c1-c1cc2nc(n1)NS(=O)(=O)c1cccc(c1)C(=O)NCC(c1ccc(Br)cc1)O2]

To a stirring solution of 3-[[4-[2-amino-1-(4-bromophenyl)ethoxy]-6-(2,6-dimethylphenyl)pyrimidin-2-yl]sulfamoyl]benzoic acid (hydrochloride salt) (5.35 g, 7.9329 mmol) in anhydrous DMF (300 mL) at room temperature under nitrogen was added DIPEA (10.253 g, 13.818 mL, 79.329 mmol), followed by HATU (3.6196 g, 9.5195 mmol). The reaction mixture was stirred for 2 h. After cooling to 0° C., the reaction mixture was quenched with 10% aqueous citric acid (500 mL). The precipitated white solid (1.012 g) was collected by filtration, washed with water (100 mL) and dried under vacuum. The remaining aqueous layer was extracted with ethyl acetate (3×150 mL). The combined organic layers were washed with brine (2×200 mL), dried over anhydrous sodium sulfate and concentrated. The resulting material was purified by reverse phase HPLC using water-acetonitrile (0.1% TFA buffer) gradient method (C₁₈ Varian column, 30-70% acetonitrile, 60 mL/min) to afford more white solid (165 mg). Both batches of solid were pure product, and were thus combined: 10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.177 g, 25%). ¹H NMR (250 MHz, DMSO-d₆) δ 8.67 (s, 1H), 8.05-7.88 (m, 2H), 7.79 (s, 7H), 7.24 (d, J 7.7 Hz, 2H), 7.13 (s, 2H), 6.36 (s, 1H), 6.30-6.07 (m, 2H), 2.05 (s, 6H). ESI-MS m/z calc. 578.0623, found 579.4 (M+1)⁺; Retention time: 2.35 minutes; LC method T.

### Example 27: Preparation of Compound 21

### Step 1: 6-(2,6-Dimethylphenyl)-10-(4-dimethylphosphorylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 21)

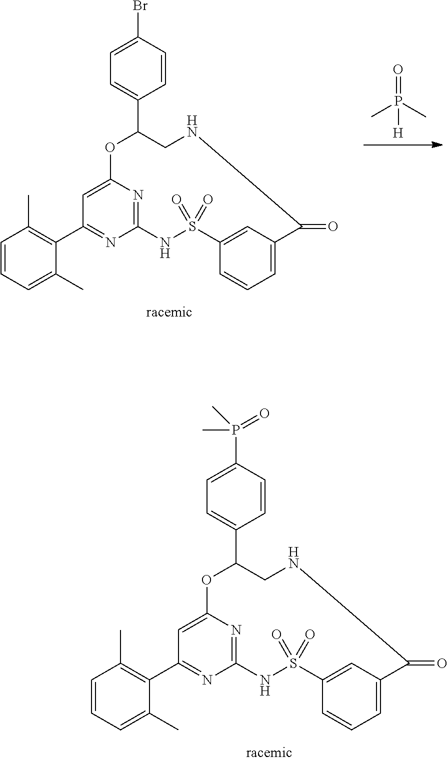

A heterogeneous solution of racemic 10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (9 mg, 0.01538 mmol), methylphosphonoylmethane (2.4 mg, 0.03075 mmol), potassium carbonate (6.4 mg, 0.04631 mmol), and CuI (0.18 mg, 9.451E-4 mmol) in DMF (50 μL) and toluene (50 μL) was stirring under microwave irradiation at 120° C. for 40 min. The solution was filtered. The sample was purified by reverse phase HPLC (C₁₈) to afford 6-(2,6-dimethylphenyl)-10-(4-dimethylphosphorylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (3.6 mg, 41%) ESI-MS m/z calc. 576.1596, found 577.5 (M+1)⁺; Retention time: 0.98 minutes; LC method A.

### Example 28: Preparation of Compound 22

### Step 1: 6-(2,6-Dimethylphenyl)-2,2-dioxo-10-[4-(1,1,2,2,2-pentafluoroethyl)phenyl]-9-oxa-2λ⁶-thia-3,5,12,19-tetrazatricyclo[12.3.1.14,8]nonadeca-1(18),4(19),5,7,14,16-hexaen-13-one (Compound 22)

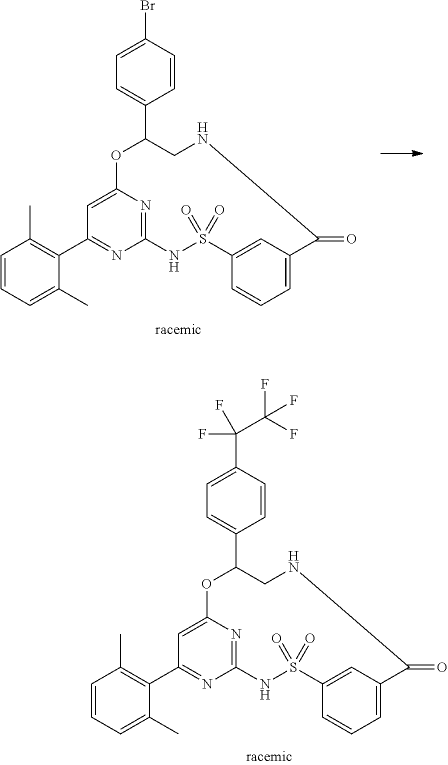 [SMILES: Cc1cccc(C)c1-c1cc2nc(n1)NS(=O)(=O)c1cccc(c1)C(=O)NCC(c1ccc(Br)cc1)O2 ; Cc1cccc(C)c1-c1cc2nc(n1)NS(=O)(=O)c1cccc(c1)C(=O)NCC(c1ccc(C(F)(F)C(F)(F)F)cc1)O2]

Activation of copper powder (−150 mesh, 9.0 gram) prior to use: 9.0 gram of bronze copper powder was stirred in 100 ml of a solution of 2% iodine in acetone for 15 minutes until the iodine solution was decolorized. The product was collected in Buchner funnel and suspended in a solution of concentrated HCl in acetone (1:1, v/v, 40 mL). After stirring for 5 minutes, it was filtered, and the solid was washed with acetone (3×20 mL) and dried for 30 min. A mixture of 10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 0.05177 mmol), activated Cu (35 mg, 0.5508 mmol) and 1,1,1,2,2-pentafluoro-2-iodo-ethane (203 mg, 0.8255 mmol) in DMSO (0.5 mL) in a sealed microwave vial was heated at 80° C. for 24 h. The reaction mixture was diluted with MeOH, filtered and purified by HPLC (1-99% ACN in water (HCl modifier)) to give 6-(2,6-dimethylphenyl)-2,2-dioxo-10-[4-(1,1,2,2,2-pentafluoroethyl)phenyl]-9-oxa-2λ⁶-thia-3,5,12,19-tetrazatricyclo[12.3.1.14,8]nonadeca-1(18),4(19),5,7,14,16-hexaen-13-one (3.8 mg, 12%) as an off-white solid. ESI-MS m/z calc. 618.13605, found 619.2 (M+1)⁺; Retention time: 1.82 minutes; LC method A.

### Example 29: Preparation of Compound 23 and Compound 24

### Step 1: 10-[4-[(E)-3,3-Dimethylbut-1-enyl]phenyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 23)

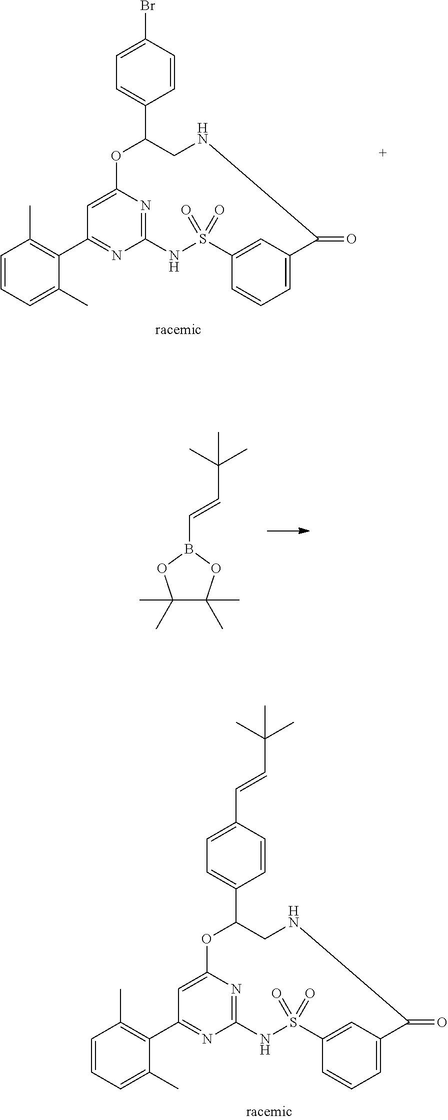 [SMILES: Cc1cccc(C)c1-c1cc2nc(n1)NS(=O)(=O)c1cccc(c1)C(=O)NCC(c1ccc(Br)cc1)O2 ; CC(C)(C)/C=C/B1OC(C)(C)C(C)(C)O1 ; Cc1cccc(C)c1-c1cc2nc(n1)NS(=O)(=O)c1cccc(c1)C(=O)NCC(c1ccc(/C=C/C(C)(C)C)cc1)O2]

To mixture of 10-(4-bromophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (40 mg, 0.06903 mmol), 2-[(E)-3,3-dimethylbut-1-enyl]-4,4,5,5-tetramethyl-1,3,2-dioxaborolane (40 mg, 0.1904 mmol), Pd(dppf)Cl₂ (6 mg, 0.008200 mmol) and potassium carbonate (138 μL of 2 M, 0.2760 mmol) in DME (400 μL) was stirred at 90° C. for 2 h. The reaction mixture was poured into water, pH brought to ˜5 with 1 N HCl and extracted with EtOAc (2×). The organic layers were combined, washed with water then brine, dried over sodium sulfate and evaporated to dryness. Purification by column chromatography (4 g silica; 1-80% EtOAc in hexanes) gave 10-[4-[(E)-3,3-dimethylbut-1-enyl]phenyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (32 mg, 80%) as a foam. ESI-MS m/z calc. 582.2301, found 583.3 (M+1)⁺; Retention time: 2.02 minutes; LC method A.

### Step 2: 10-[4-(3,3-Dimethylbutyl)phenyl]-6-(2,6-dimethylphenyl)-2,2-dioxo-9-oxa-2)⁶-thia-3,5,12,19-tetrazatricyclo[12.3.1.14,8]nonadeca-1(18),4(19),5,7,14,16-hexaen-13-one (Compound 24)

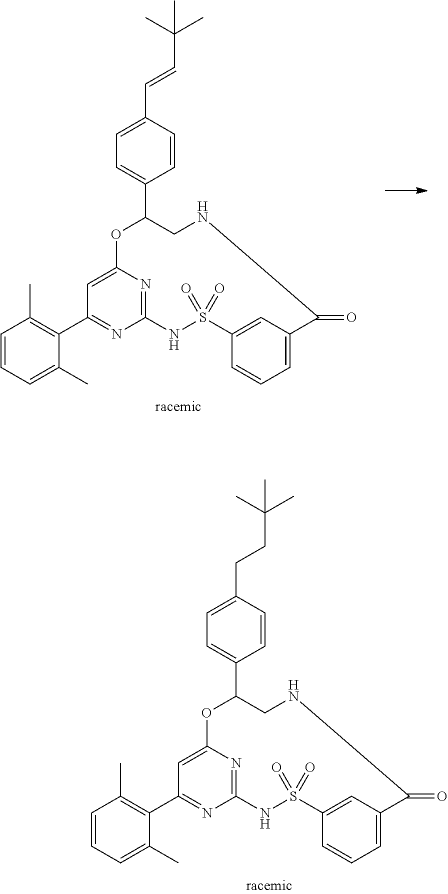 [SMILES: Cc1cccc(C)c1-c1cc2nc(n1)NS(=O)(=O)c1cccc(c1)C(=O)NCC(c1ccc(/C=C/C(C)(C)C)cc1)O2 ; Cc1cccc(C)c1-c1cc2nc(n1)NS(=O)(=O)c1cccc(c1)C(=O)NCC(c1ccc(CCC(C)(C)C)cc1)O2]

A suspension of 10% w/w Pd/C (5 mg, 0.04698 mmol) and 10-[4-[(E)-3,3-dimethylbut-1-enyl]phenyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (23 mg, 0.03947 mmol) in MeOH (1.5 mL) was stirred under a balloon of hydrogen for 45 min. The reaction mixture was filtered then evaporated, then the residue was taken up in 1:1 MeOH:DMSO, filtered and purified by reverse-phase HPLC (C₁₈) to give 10-[4-(3,3-dimethylbutyl)phenyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (8.7 mg, 38%). ESI-MS m/z calc. 584.2457, found 585.2 (M+1)⁺; Retention time: 2.08 minutes; LC method A.

### Example 30: Preparation of Compound 25

### Step 1: tert-Butyl N-[(2R)-2-hydroxy-2-phenyl-ethyl]carbamate

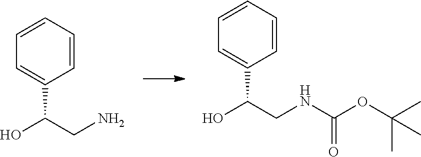 [SMILES: NC[C@H](O)c1ccccc1 ; CC(C)(C)OC(=O)NC[C@H](O)c1ccccc1]

(1R)-2-Amino-1-phenyl-ethanol (100 mg, 0.7290 mmol), Boc anhydride (approximately 175.0 mg, 184.2 μL, 0.8019 mmol), and cesium carbonate (approximately 261.3 mg, 0.8019 mmol) were combined in THF, and stirred at room temperature for 3 h. The reaction mixture was then diluted with ethyl acetate and 0.5 M HCl, and the layers were separated. The aqueous was extracted 2×ethyl acetate, washed with brine, dried over sodium sulfate and concentrated to give tert-butyl N-[(2R)-2-hydroxy-2-phenyl-ethyl]carbamate (181 mg, 105%). ESI-MS m/z calc. 237.13649, found 238.2 (M+1)⁺; Retention time: 0.49 minutes; LC method D. The product was used in the next step without further purification.

### Step 2: 3-[[4-[(1R)-2-(tert-Butoxycarbonylamino)-1-phenyl-ethoxy]-6-(2,6-dimethylphenyl)pyrimidin-2-yl]sulfamoyl]benzoic Acid

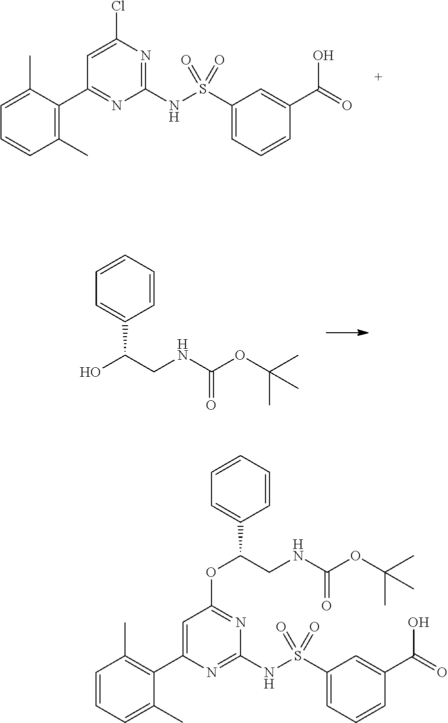 [SMILES: Cc1cccc(C)c1-c1cc(Cl)nc(NS(=O)(=O)c2cccc(C(=O)O)c2)n1 ; CC(C)(C)OC(=O)NC[C@H](O)c1ccccc1 ; Cc1cccc(C)c1-c1cc(O[C@@H](CNC(=O)OC(C)(C)C)c2ccccc2)nc(NS(=O)(=O)c2cccc(C(=O)O)c2)n1]

tert-Butyl N-[(2R)-2-hydroxy-2-phenyl-ethyl]carbamate (approximately 221.5 mg, 0.9336 mmol), 3-[[4-chloro-6-(2,6-dimethylphenyl)pyrimidin-2-yl]sulfamoyl]benzoic acid (65 mg, 0.1556 mmol), and sodium tert-butoxide (approximately 149.5 mg, 1.556 mmol) were combined in THF, and stirred at room temperature for 16 h. The reaction mixture was then poured into ethyl acetate and 1 N HCl, and the layers were separated. The aqueous was extracted 3 additional times with ethyl acetate, and the combined organics were washed with brine, dried over sodium sulfate and concentrated. The resulting crude material was dissolved in 1:1 DMSO/methanol, filtered and purified by preparative reverse-phase HPLC (C₁₈) to give 3-[[4-[(1R)-2-(tert-butoxycarbonylamino)-1-phenyl-ethoxy]-6-(2,6-dimethylphenyl)pyrimidin-2-yl]sulfamoyl]benzoic acid (16 mg, 17%). ESI-MS m/z calc. 618.21484, found 619.3 (M+1)⁺; Retention time: 0.7 minutes; LC method D.

### Step 3: (10R)-6-(2,6-Dimethylphenyl)-2,2-dioxo-10-phenyl-9-oxa-2λ⁶-thia-3,5,12,19-tetrazatricyclo[12.3.1.14,8]nonadeca-1(18),4(19),5,7,14,16-hexaen-13-one (Compound 25)

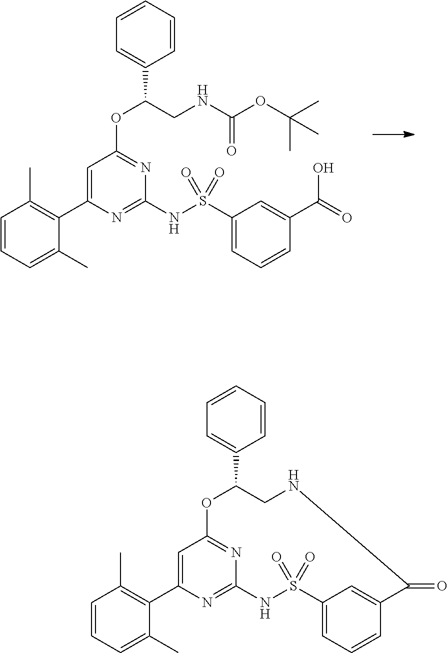 [SMILES: Cc1cccc(C)c1-c1cc(O[C@@H](CNC(=O)OC(C)(C)C)c2ccccc2)nc(NS(=O)(=O)c2cccc(C(=O)O)c2)n1 ; Cc1cccc(C)c1-c1cc2nc(n1)NS(=O)(=O)c1cccc(c1)C(=O)NC[C@@H](c1ccccc1)O2]

Stage 1: 3-[[4-[(1R)-2-(tert-Butoxycarbonylamino)-1-phenyl-ethoxy]-6-(2,6-dimethylphenyl)pyrimidin-2-yl]sulfamoyl]benzoic acid (16 mg, 0.02586 mmol) was dissolved in DCM (0.25 mL) and HCl in dioxane (approximately 161.6 μL of 4 M, 0.6465 mmol) was added. After stirring for 1 h at room temperature, the reaction mixture was evaporated, and concentrated to give the Boc-deprotected amine, which was used in the next stage without further purification.

Stage 2: The amine product from Stage 1 was combined with HATU (approximately 12.78 mg, 0.03362 mmol) in DMF (1 mL), and DIPEA (approximately 16.71 mg, 22.52 μL, 0.1293 mmol) was added. The reaction mixture was stirred at room temperature for 1 h. After this time, the reaction mixture was filtered, and purified by preparative reverse-phase HPLC (C₁₈) to give (10R)-6-(2,6-dimethylphenyl)-2,2-dioxo-10-phenyl-9-oxa-2λ⁶-thia-3,5,12,19-tetrazatricyclo[12.3.1.14,8]nonadeca-1(18),4(19),5,7,14,16-hexaen-13-one (2.7 mg, 23%); ESI-MS m/z calc. 500.15182, found 501.3 (M+1)⁺; Retention time: 1.42 minutes; LC method A.

### Example 31: Preparation of Compound 26

### Step 1: tert-Butyl N-[(2S)-2-hydroxy-2-phenyl-ethyl]carbamate

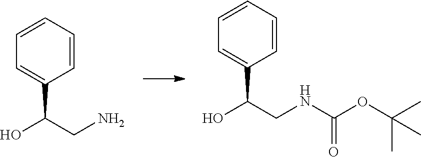 [SMILES: NC[C@@H](O)c1ccccc1 ; CC(C)(C)OC(=O)NC[C@@H](O)c1ccccc1]

(1S)-2-amino-1-phenyl-ethanol (100 mg, 0.7290 mmol), Boc anhydride (approximately 175.0 mg, 184.2 μL, 0.8019 mmol), and cesium carbonate (approximately 261.3 mg, 0.8019 mmol) were combined in THF, and stirred at room temperature for 3 h. The reaction mixture was then diluted with ethyl acetate and 0.5 M HCl, and the layers were separated. The aqueous was extracted 2×ethyl acetate, washed with brine, dried over sodium sulfate and concentrated to give tert-butyl N-[(2S)-2-hydroxy-2-phenyl-ethyl]carbamate (186 mg, 108%). ESI-MS m/z calc. 237.13649, found 238.2 (M+1)⁺; Retention time: 0.5 minutes; LC method A. The product was used in the next step without further purification.

### Step 2: 3-[[4-[(1S)-2-(tert-Butoxycarbonylamino)-1-phenyl-ethoxy]-6-(2,6-dimethylphenyl)pyrimidin-2-yl]sulfamoyl]benzoic Acid

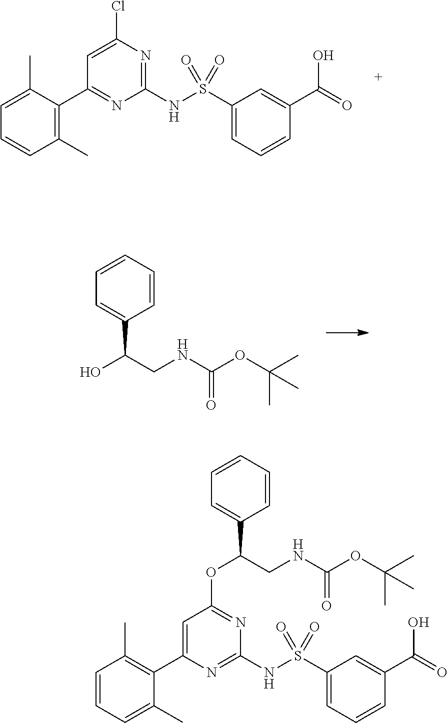 [SMILES: Cc1cccc(C)c1-c1cc(Cl)nc(NS(=O)(=O)c2cccc(C(=O)O)c2)n1 ; CC(C)(C)OC(=O)NC[C@@H](O)c1ccccc1 ; Cc1cccc(C)c1-c1cc(O[C@H](CNC(=O)OC(C)(C)C)c2ccccc2)nc(NS(=O)(=O)c2cccc(C(=O)O)c2)n1]

tert-Butyl N-[(2S)-2-hydroxy-2-phenyl-ethyl]carbamate (approximately 221.5 mg, 0.9336 mmol), 3-[[4-chloro-6-(2,6-dimethylphenyl)pyrimidin-2-yl]sulfamoyl]benzoic acid (65 mg, 0.1556 mmol), and sodium tert-butoxide (approximately 149.5 mg, 1.556 mmol) were combined in THF, and stirred at room temperature for 16 h. The reaction mixture was then poured into ethyl acetate and 1 N HCl, and the layers were separated. The aqueous was extracted 3 additional times with ethyl acetate, and the combined organics were washed with brine, dried over sodium sulfate and concentrated. The resulting crude material was dissolved in 1:1 DMSO/methanol, filtered and purified by preparative reverse-phase HPLC (C₁₈) to give 3-[[4-[(1S)-2-(tert-butoxycarbonylamino)-1-phenyl-ethoxy]-6-(2,6-dimethylphenyl)pyrimidin-2-yl]sulfamoyl]benzoic acid (25 mg, 26%). ESI-MS m/z calc. 618.21484, found 619.3 (M+1)⁺; Retention time: 0.7 minutes; LC method D.

### Step 3: (10S)-6-(2,6-Dimethylphenyl)-2,2-dioxo-10-phenyl-9-oxa-2λ⁶-thia-3,5,12,19-tetrazatricyclo[12.3.1.14,8]nonadeca-1(18),4(19),5,7,14,16-hexaen-13-one (Compound 26)

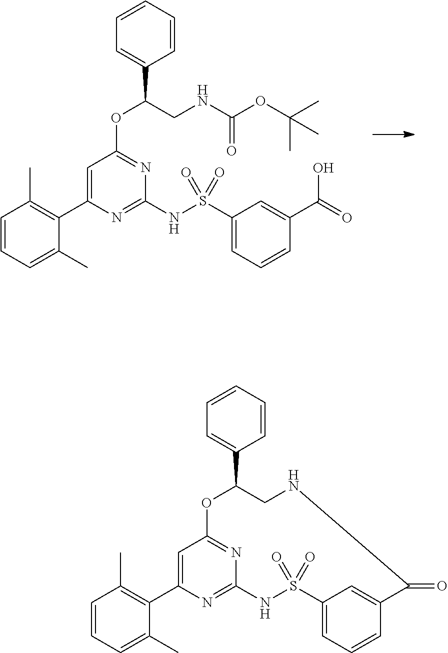 [SMILES: Cc1cccc(C)c1-c1cc(O[C@H](CNC(=O)OC(C)(C)C)c2ccccc2)nc(NS(=O)(=O)c2cccc(C(=O)O)c2)n1 ; Cc1cccc(C)c1-c1cc2nc(n1)NS(=O)(=O)c1cccc(c1)C(=O)NC[C@H](c1ccccc1)O2]

Stage 1: 3-[[4-[(1S)-2-(tert-Butoxycarbonylamino)-1-phenyl-ethoxy]-6-(2,6-dimethylphenyl)pyrimidin-2-yl]sulfamoyl]benzoic acid (16 mg, 0.02586 mmol) was dissolved in DCM (0.25 mL) and HCl in dioxane (approximately 161.6 μL of 4 M, 0.6465 mmol) was added. After stirring for 1 h at room temperature, the reaction mixture was evaporated, and concentrated to give the Boc-deprotected amine, which was used in the next stage without further purification.

Stage 2: The amine product from Stage 1 was combined with HATU (approximately 12.78 mg, 0.03362 mmol) in DMF (1 mL), and DIPEA (approximately 16.71 mg, 22.52 μL, 0.1293 mmol) was added. The reaction mixture was stirred at room temperature for 1 h. After this time, the reaction mixture was filtered, and purified by preparative reverse-phase HPLC (C₁₈) to give (10S)-6-(2,6-dimethylphenyl)-2,2-dioxo-10-phenyl-9-oxa-2λ⁶-thia-3,5,12,19-tetrazatricyclo[12.3.1.14,8]nonadeca-1(18),4(19),5,7,14,16-hexaen-13-one (2.5 mg, 38%); ESI-MS m/z calc. 500.15182, found 501.3 (M+1)⁺; Retention time: 1.42 minutes; LC method A.

### Example 32: Preparation of Compound 27

### Step 1: tert-Butyl (S)-(2-hydroxypropyl)carbamate

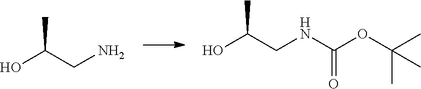

(2S)-1-Aminopropan-2-ol (100 mg, 1.331 mmol), Boc anhydride (approximately 319.5 mg, 336.3 μL, 1.464 mmol), and cesium carbonate (approximately 477.0 mg, 1.464 mmol) were combined in THF, and stirred at room temperature for 3 h. The reaction mixture was then diluted with ethyl acetate and 0.5 M HCl, and the layers were separated. The aqueous was extracted 2× ethyl acetate, washed with brine, dried over sodium sulfate and concentrated to give tert-butyl (S)-(2-hydroxypropyl)carbamate (248 mg, 106%); ESI-MS m/z calc. 175.12085, found 176.2 (M+1)⁺; Retention time: 0.34 minutes; LC method A. The product was used in the next step without further purification.

### Step 2: 3-[[4-[(1S)-2-(tert-Butoxycarbonylamino)-1-methyl-ethoxy]-6-(2,6-dimethylphenyl)pyrimidin-2-yl]sulfamoyl]benzoic Acid

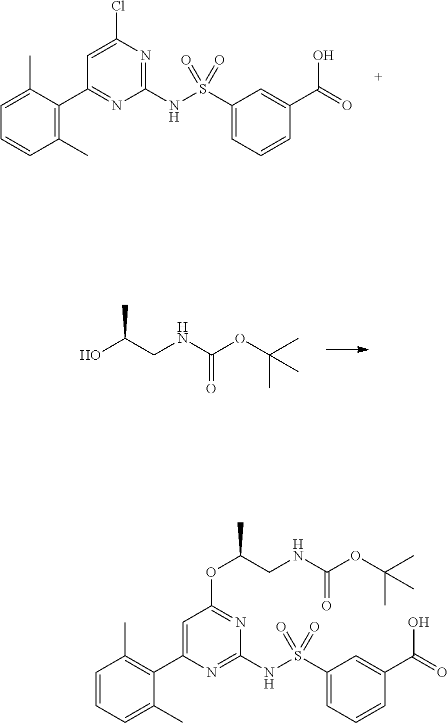 [SMILES: Cc1cccc(C)c1-c1cc(Cl)nc(NS(=O)(=O)c2cccc(C(=O)O)c2)n1 ; C[C@H](O)CNC(=O)OC(C)(C)C ; Cc1cccc(C)c1-c1cc(O[C@@H](C)CNC(=O)OC(C)(C)C)nc(NS(=O)(=O)c2cccc(C(=O)O)c2)n1]

tert-Butyl (S)-(2-hydroxypropyl)carbamate (approximately 163.6 mg, 0.9336 mmol), 3-[[4-chloro-6-(2,6-dimethylphenyl)pyrimidin-2-yl]sulfamoyl]benzoic acid (65 mg, 0.1556 mmol), and sodium tert-butoxide (approximately 149.5 mg, 1.556 mmol) were combined in THF, and stirred at room temperature for 16 h. The reaction mixture was then poured into ethyl acetate and 1 N HCl, and the layers were separated. The aqueous was extracted 3 additional times with ethyl acetate, and the combined organics were washed with brine, dried over sodium sulfate and concentrated. The resulting crude material was dissolved in 1:1 DMSO/methanol, filtered and purified by preparative reverse-phase HPLC (C₁₈) to 3-[[4-[(1S)-2-(tert-butoxycarbonylamino)-1-methyl-ethoxy]-6-(2,6-dimethylphenyl)pyrimidin-2-yl]sulfamoyl]benzoic acid (8 mg, 9%). ESI-MS m/z calc. 556.19916, found 557.3 (M+1)⁺; Retention time: 0.61 minutes; LC method D.

### Step 3: (10S)-6-(2,6-Dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 27)

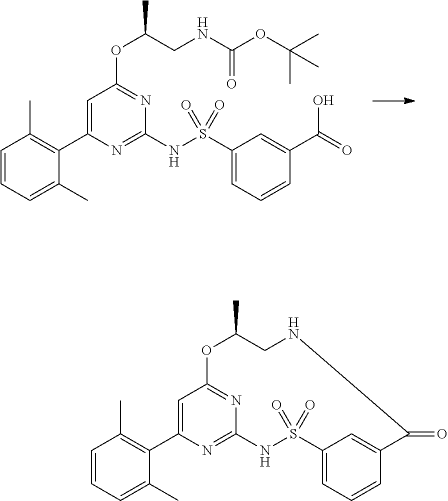 [SMILES: Cc1cccc(C)c1-c1cc(O[C@@H](C)CNC(=O)OC(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)NC[C@H](C)O2]

Stage 1: 3-[[4-[(1S)-2-(tert-butoxycarbonylamino)-1-methyl-ethoxy]-6-(2,6-dimethylphenyl)pyrimidin-2-yl]sulfamoyl]benzoic acid (8 mg, 0.01437 mmol) was dissolved in DCM (0.25 mL) and HCl in dioxane (approximately 89.80 μL of 4 M, 0.3592 mmol) was added. After stirring for 1 h at room temperature, the reaction mixture was evaporated, and concentrated to give the Boc-deprotected amine, which was used in the next stage without further purification.

Stage 2: The amine product from Stage 1 was combined with HATU (approximately 7.103 mg, 0.01868 mmol) in DMF (1 mL), and DIPEA (approximately 9.286 mg, 12.51 μL, 0.07185 mmol) was added. The reaction mixture was stirred at room temperature for 1 h. After this time, the reaction mixture was filtered, and purified by preparative reverse-phase HPLC (C₁₈) to give (10S)-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (3.2 mg, 55%); ESI-MS m/z calc. 438.13617, found 439.3 (M+1)⁺; Retention time: 1.15 minutes; LC method A.

### Example 33: Preparation of Compound 28

### Step 1: 3-[[4-[(1R)-2-(tert-Butoxycarbonylamino)-1-methyl-ethoxy]-6-(2,6-dimethylphenyl)pyrimidin-2-yl]sulfamoyl]benzoic Acid

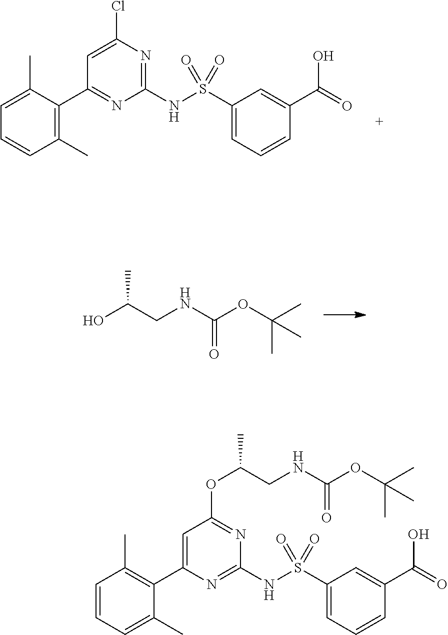 [SMILES: Cc1cccc(C)c1-c1cc(Cl)nc(NS(=O)(=O)c2cccc(C(=O)O)c2)n1 ; C[C@@H](O)CNC(=O)OC(C)(C)C ; Cc1cccc(C)c1-c1cc(O[C@H](C)CNC(=O)OC(C)(C)C)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (20.03 g, 44.579 mmol) and tert-butyl N-[(2R)-2-hydroxypropyl]carbamate (10.86 g, 59.498 mmol) in anhydrous THE (380 mL) stirring at 0° C. was added sodium tert-butoxide (26.3262 g, 268.46 mmol) portion-wise. The reaction was allowed to stir at 0° C. for 1 h and then warmed to RT for 20 h. The reaction was quenched, and the pH was adjusted to 3 with aqueous 10% citric acid (600 mL) and stirred for 5 min. The mixture was extracted with EtOAc (3×250 mL). The combined organic layers were washed with saturated aqueous NaCl (100 mL), dried over anhydrous sodium sulfate, filtered and concentrated under vacuum. The crude product was subjected to flash chromatography (330 g silica gel, eluting 0 to 50% acetone/hexanes buffered with 0.1% acetic acid). Appropriate fractions were collected and concentrated under vacuum. Mixed fractions containing the desired product were combined, concentrated under vacuum, and subjected to flash chromatography by (80 g silica gel, eluting with 30% acetone/hexanes buffered with 0.1% acetic acid). Appropriate fractions were collected and concentrated under vacuum. Total product: 3-[[4-[(1R)-2-(tert-butoxycarbonylamino)-1-methyl-ethoxy]-6-(2,6-dimethylphenyl)pyrimidin-2-yl]sulfamoyl]benzoic acid (3.81 g, 15%)¹H NMR (250 MHz, DMSO-d₆) δ 8.42 (s, 1H), 8.17-7.99 (m, 2H), 7.68 (t, J 7.8 Hz, 1H), 7.32-7.19 (m, 1H), 7.13 (d, J 7.5 Hz, 2H), 6.99-6.90 (m, 1H), 6.15 (s, 1H), 4.96-4.79 (m, 1H), 4.72-4.55 (m, 1H), 3.61-3.49 (m, 1H), 3.11-2.96 (m, 2H), 2.07 (s, 6H), 1.40-1.27 (m, 9H), 1.21-1.06 (m, 3H). ESI-MS m/z calc. 556.19916, found 557.2 (M+1)⁺; Retention time: 5.1 minutes; LC method S.

### Step 2: (10R)-6-(2,6-Dimethylphenyl)-10-methyl-2,2-dioxo-9-oxa-2⁶-thia-3,5,12,19-tetrazatricyclo[12.3.1.14,8]nonadeca-1(18),4(19),5,7,14,16-hexaen-13-one ((Compound 28)

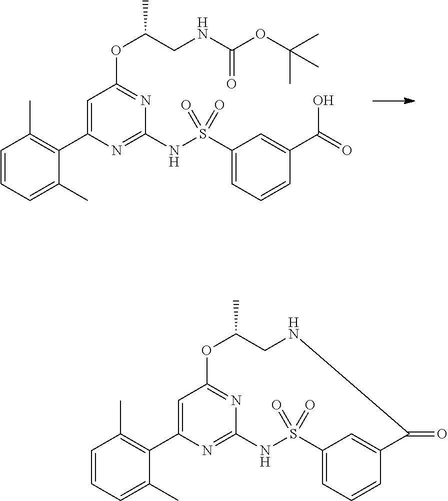 [SMILES: Cc1cccc(C)c1-c1cc(O[C@H](C)CNC(=O)OC(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)NC[C@@H](C)O2]

Stage 1: 3-[[4-[(1R)-2-(tert-Butoxycarbonylamino)-1-methyl-ethoxy]-6-(2,6-dimethylphenyl)pyrimidin-2-yl]sulfamoyl]benzoic acid (26 mg, 0.04671 mmol) was dissolved in DCM (0.25 mL) and HCl in dioxane (approximately 292.0 μL of 4 M, 1.168 mmol) was added. After stirring for 1 h at room temperature, the reaction mixture was evaporated, and concentrated to give the Boc-deprotected amine, which was used in the next stage without further purification.

Stage 2: The amine product from Stage 1 was combined with HATU (approximately 23.09 mg, 0.06072 mmol) in DMF (1 mL), and DIPEA (approximately 30.18 mg, 40.67 μL, 0.2335 mmol) was added. The reaction mixture was stirred at room temperature for 1 h. After this time, the reaction mixture was filtered, and purified by preparative reverse-phase HPLC (C₁₈) to give (10R)-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (9.8 mg, 46%); ESI-MS m/z calc. 438.13617, found 439.2 (M+1)⁺; Retention time: 1.15 minutes; LC method A.

### Example 34: Preparation of Compound 29

### Step 1: tert-Butyl N-[(2R)-2,3-dihydroxypropyl]carbamate

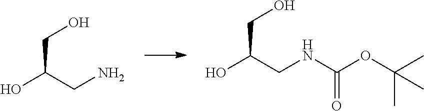

(2R)-3-Aminopropane-1,2-diol (200 mg, 2.195 mmol), Boc anhydride (approximately 526.8 mg, 554.5 μL, 2.414 mmol), and cesium carbonate (approximately 786.5 mg, 2.414 mmol) were combined in THF, and stirred at room temperature for 3 h. The reaction mixture was then diluted with ethyl acetate and 0.5 M HCl, and the layers were separated. The aqueous was extracted 2×ethyl acetate, washed with brine, dried over sodium sulfate and concentrated to give tert-butyl N-[(2R)-2,3-dihydroxypropyl]carbamate (461 mg, 110%). ESI-MS m/z calc. 191.11575, found 192.2 (M+1)⁺; Retention time: 0.28 minutes; LC method D. The product was used in the next step without further purification.

### Step 2: tert-Butyl N-[(2R)-3-[tert-butyl(diphenyl)silyl]oxy-2-hydroxy-propyl]carbamate

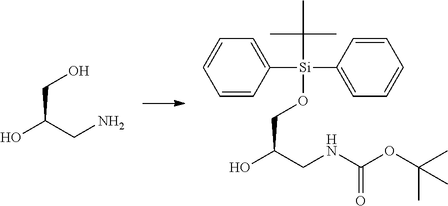 [SMILES: NC[C@@H](O)CO ; CC(C)(C)OC(=O)NC[C@@H](O)CO[Si](c1ccccc1)(c1ccccc1)C(C)(C)C]

tert-Butyl N-[(2R)-2,3-dihydroxypropyl]carbamate (260 mg, 1.360 mmol) and imidazole (approximately 185.2 mg, 2.720 mmol) were combined in anhydrous dichloromethane (0.5 mL). The reaction mixture was then cooled to 0° C., tert-butyl-chloro-diphenyl-silane (approximately 355.1 mg, 336.0 μL, 1.292 mmol) was added, and the reaction mixture was allowed to stir at room temperature for 16 h. The reaction mixture was then diluted with ethyl acetate, washed with 1 N HCl, brine, dried over sodium sulfate and concentrated. The resulting tert-butyl N-[(2R)-3-[tert-butyl(diphenyl)silyl]oxy-2-hydroxy-propyl]carbamate (417 mg, 71%) was used in the next step without purification. ESI-MS m/z calc. 429.23352, found 430.4 (M+1)⁺; Retention time: 0.84 minutes; LC method D.

### Step 3: 3-[[4-[(1R)-1-[(tert-Butoxycarbonylamino)methyl]-2-[tert-butyl(diphenyl)silyl]oxy-ethoxy]-6-(2,6-dimethylphenyl)pyrimidin-2-yl]sulfamoyl]benzoic Acid

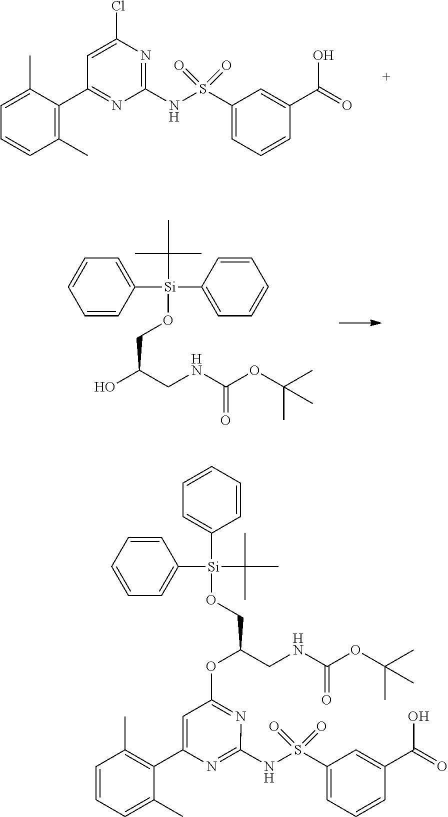 [SMILES: Cc1cccc(C)c1-c1cc(Cl)nc(NS(=O)(=O)c2cccc(C(=O)O)c2)n1 ; CC(C)(C)OC(=O)NC[C@@H](O)CO[Si](c1ccccc1)(c1ccccc1)C(C)(C)C ; Cc1cccc(C)c1-c1cc(O[C@H](CNC(=O)OC(C)(C)C)CO[Si](c2ccccc2)(c2ccccc2)C(C)(C)C)nc(NS(=O)(=O)c2cccc(C(=O)O)c2)n1]

tert-Butyl N-[(2R)-3-[tert-butyl(diphenyl)silyl]oxy-2-hydroxy-propyl]carbamate (approximately 401.1 mg, 0.9336 mmol), 3-[[4-chloro-6-(2,6-dimethylphenyl)pyrimidin-2-yl]sulfamoyl]benzoic acid (65 mg, 0.1556 mmol), and sodium tert-butoxide (approximately 149.5 mg, 1.556 mmol) were combined in THF, and stirred at room temperature for 16 hours. The reaction mixture was then poured into ethyl acetate and 1 N HCl, and the layers were separated. The aqueous was extracted 3 additional times with ethyl acetate, and the combined organics were washed with brine, dried over sodium sulfate and concentrated. The resulting crude material was dissolved in 1:1 DMSO/methanol, filtered and purified by preparative reverse-phase HPLC (C₁₈) to give 3-[[4-[(1R)-1-[(tert-butoxycarbonylamino)methyl]-2-[tert-butyl(diphenyl)silyl]oxy-ethoxy]-6-(2,6-dimethylphenyl)pyrimidin-2-yl]sulfamoyl]benzoic Acid (46 mg, 36%). ESI-MS m/z calc. 810.3119, found 811.5 (M+1)⁺; Retention time: 0.89 minutes; LC method D.

### Step 4: (10R)-10-[[tert-Butyl(diphenyl)silyl]oxymethyl]-6-(2,6-dimethylphenyl)-2,2-dioxo-9-oxa-2)⁶-thia-3,5,12,19-tetrazatricyclo[12.3.1.14,8]nonadeca-1(18),4(19),5,7,14,16-hexaen-13-one

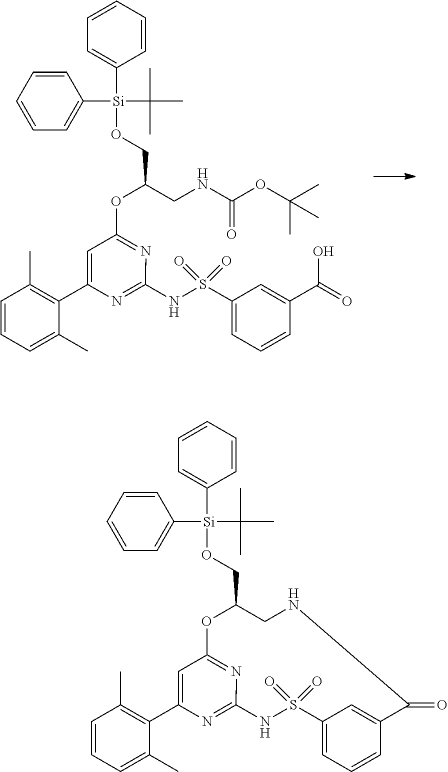 [SMILES: Cc1cccc(C)c1-c1cc(O[C@H](CNC(=O)OC(C)(C)C)CO[Si](c2ccccc2)(c2ccccc2)C(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)NC[C@H](CO[Si](c1ccccc1)(c1ccccc1)C(C)(C)C)O2]

Stage 1: 3-[[4-[(1R)-1-[(tert-butoxycarbonylamino)methyl]-2-[tert-butyl(diphenyl)silyl]oxy-ethoxy]-6-(2,6-dimethylphenyl)pyrimidin-2-yl]sulfamoyl]benzoic acid (50 mg, 0.06165 mmol) was dissolved in DCM (0.25 mL) and HCl in dioxane (approximately 385.2 μL of 4 M, 1.541 mmol) was added. After stirring for 1 hour at room temperature, the reaction mixture was evaporated, and concentrated to give the Boc-deprotected amine, which was used in the next stage without further purification.

Stage 2: The amine product from Stage 1 was combined with HATU (approximately 30.47 mg, 0.08014 mmol) in DMF (2 mL), and DIPEA (approximately 39.83 mg, 53.68 μL, 0.3082 mmol) was added. The reaction mixture was stirred at room temperature for 1 h. After this time, the reaction mixture was filtered, and purified by preparative reverse-phase HPLC (C₁₈) to give (10R)-10-[[tert-butyl(diphenyl)silyl]oxymethyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (42 mg, 98%); ESI-MS m/z calc. 692.24884, found 693.4 (M+1)⁺; Retention time: 0.83 minutes; LC method D.

### Step 5: (10R)-6-(2,6-Dimethylphenyl)-10-(hydroxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 29)

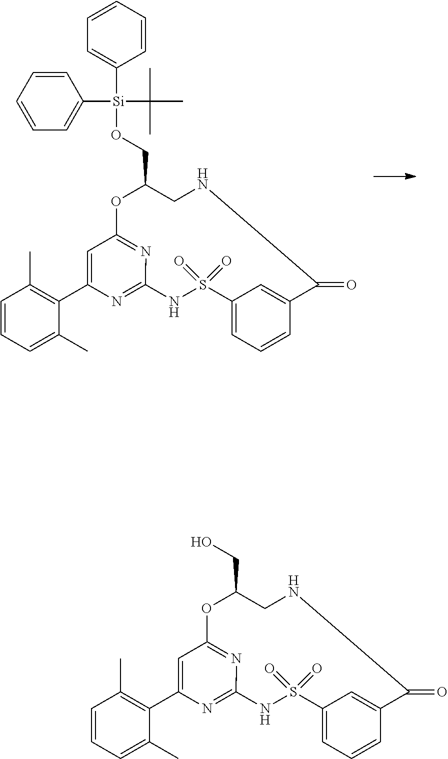 [SMILES: Cc1cccc(C)c1-c1cc2nc(n1)NS(=O)(=O)c1cccc(c1)C(=O)NC[C@H](CO[Si](c1ccccc1)(c1ccccc1)C(C)(C)C)O2 ; Cc1cccc(C)c1-c1cc2nc(n1)NS(=O)(=O)c1cccc(c1)C(=O)NC[C@H](CO)O2]

(10R)-10-[[tert-Butyl(diphenyl)silyl]oxymethyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (42 mg, 0.06061 mmol) was combined with TBAF (approximately 60.61 μL of 1 M, 0.06061 mmol) in THE (0.5 mL) and stirred at room temperature for 2 h. At this point, a second portion of TBAF (approximately 303.0 μL of 1 M, 0.3030 mmol) was added and the reaction was stirred for additional 4 h at room temperature. The reaction mixture was then partitioned between aqueous ammonium chloride and ethyl acetate. The layers were separated and the aqueous was extracted an additional 3× with ethyl acetate. The combined organics were washed with brine, dried over sodium sulfate and concentrated. The resulting crude was purified by reverse-phase HPLC (C₁₈) to give (10R)-6-(2,6-dimethylphenyl)-10-(hydroxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (10.9 mg, 39%); ESI-MS m/z calc. 454.1311, found 455.3 (M+1)⁺; Retention time: 0.93 minutes; LC method A.

### Example 35: Preparation of Compound 30

### Step 1: tert-Butyl N-[3-(3,3-dimethylbutylamino)-2-hydroxy-3-oxo-propyl]carbamate

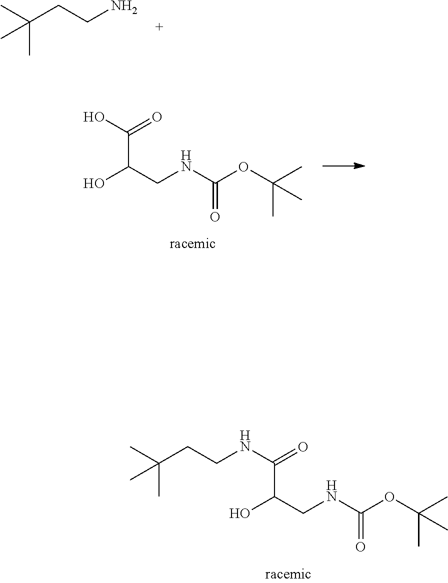

In a 250-mL flask, racemic 3-(tert-butoxycarbonylamino)-2-hydroxy-propanoic acid (500 mg, 2.437 mmol) was dissolved in DMF (15.0 mL), to which DIPEA (2.2 mL, 12.63 mmol) and HATU (1.12 g, 2.946 mmol) were added. After 5 min, 3,3-dimethylbutan-1-amine (330 μL, 2.452 mmol) was added. After stirring at room temperature for 20 h, the mixture was diluted with aqueous 10% citric acid solution and extracted with ethyl acetate (3×25 mL). The combined organic extract was washed then with aqueous sodium bicarbonate (25 mL) and saturated brine solution (50 mL), extracted organic then dried over sodium sulfate, filtered, and evaporated in vacuo. This crude product was purified on silica gel chromatography (80 gram column) using a gradient from 100% dichloromethane to 10% methanol in dichloromethane followed by a second silica gel chromatography (24 gram column) using a gradient from 100% hexanes to 60% ethyl acetate in hexanes. A third column was required utilizing silica gel chromatography (40 gram column) using a shallow gradient from 100% dichloromethane to 5% methanol in dichloromethane to afford a white solid, tert-butyl N-[3-(3,3-dimethylbutylamino)-2-hydroxy-3-oxo-propyl]carbamate (119 mg, 17%); ¹H NMR (400 MHz, Chloroform-d) δ 6.93 (s, 1H), 5.46-4.97 (m, 2H), 4.16 (td, J 5.0, 2.5 Hz, 1H), 3.71-3.39 (m, 2H), 3.40-3.13 (m, 2H), 1.61 (s, 2H), 1.45 (d, J 2.7 Hz, 9H), 0.94 (s, 9H). ESI-MS m/z calc. 288.2049, found 289.3 (M+1)⁺; Retention time: 1.34 minutes; LC method A.

### Step 2: 3-[[4-[1-(Aminomethyl)-2-(3,3-dimethylbutylamino)-2-oxo-ethoxy]-6-(2,6-dimethylphenyl)pyrimidin-2-yl]sulfamoyl]benzoic Acid

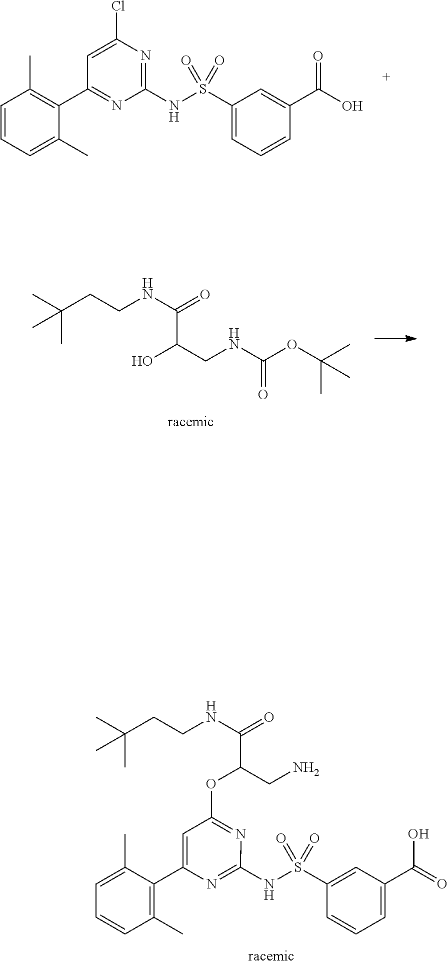 [SMILES: Cc1cccc(C)c1-c1cc(Cl)nc(NS(=O)(=O)c2cccc(C(=O)O)c2)n1 ; CC(C)(C)CCNC(=O)C(O)CNC(=O)OC(C)(C)C ; Cc1cccc(C)c1-c1cc(OC(CN)C(=O)NCCC(C)(C)C)nc(NS(=O)(=O)c2cccc(C(=O)O)c2)n1]

In a 50-mL flask, 3-[[4-chloro-6-(2,6-dimethylphenyl)pyrimidin-2-yl]sulfamoyl]benzoic acid (160 mg, 0.3829 mmol), tert-butyl N-[3-(3,3-dimethylbutylamino)-2-hydroxy-3-oxo-propyl]carbamate (111 mg, 0.3849 mmol) and THE (4.0 mL) were mixed, to which KOtBu (260 mg, 2.317 mmol) was added. This mixture was stirred at room temperature for 1 h. The mixture was filtered and purified by reverse-phase preparative HPLC (C₁₈) column. The purified intermediate was then subjected to HCl in dioxane (1.5 mL of 4 M, 6.0 mmol) and then directly concentrated to afford a white solid, 3-[[4-[1-(aminomethyl)-2-(3,3-dimethylbutylamino)-2-oxo-ethoxy]-6-(2,6-dimethylphenyl)pyrimidin-2-yl]sulfamoyl]benzoic acid (hydrochloride salt) (55 mg, 24%); ESI-MS m/z calc. 569.23083, found 570.3 (M+1)⁺; Retention time: 1.18 minutes; LC method A.

### Step 3: N-(3,3-Dimethylbutyl)-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaene-10-carboxamide (Compound 30)

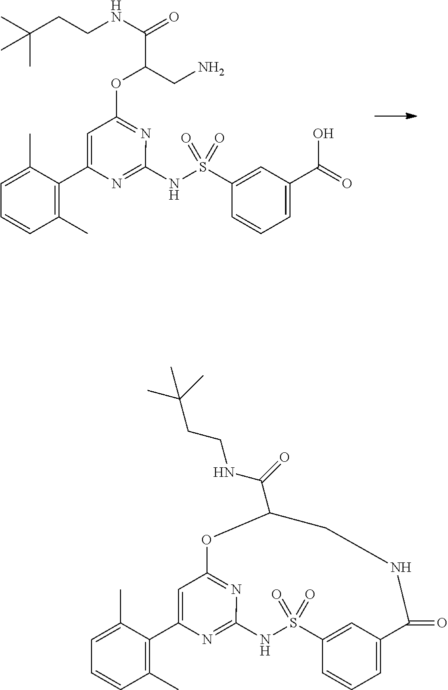 [SMILES: Cc1cccc(C)c1-c1cc(OC(CN)C(=O)NCCC(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)NCC(C(=O)NCCC(C)(C)C)O2]

In a 50-mL flask, 3-[[4-[1-(aminomethyl)-2-(3,3-dimethylbutylamino)-2-oxo-ethoxy]-6-(2,6-dimethylphenyl)pyrimidin-2-yl]sulfamoyl]benzoic acid (hydrochloride salt) (55 mg, 0.09074 mmol) was dissolved in DMF (3.0 mL), to which DIPEA (80 μL, 0.4593 mmol) and HATU (42 mg, 0.1105 mmol) were added. After stirring at room temperature for 15 min, the mixture was filtered and was purified by reverse-phase preparative HPLC (C₁₈) to afford an off-white solid, N-(3,3-dimethylbutyl)-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaene-10-carboxamide (14.2 mg, 28%)¹H NMR (400 MHz, DMSO-d₆) δ 13.04 (s, 1H), 8.80 (t, J=5.3 Hz, 1H), 8.66 (s, 1H), 7.99-7.87 (m, 1H), 7.84-7.73 (m, 1H), 7.66 (s, 2H), 7.26 (t, J 7.6 Hz, 1H), 7.12 (d, J 7.6 Hz, 2H), 6.32 (s, 1H), 5.62 (dd, J 9.3, 4.7 Hz, 1H), 3.21 (dtt, J 13.3, 8.1, 4.0 Hz, 4H), 2.04 (s, 6H), 1.52-1.39 (m, 2H), 0.94 (s, 9H). ESI-MS m/z calc. 551.2202, found 552.4 (M+1)⁺; Retention time: 1.53 minutes; LC method A.

### Example 36: Preparation of Compound 31

### Step 1: 2-Amino-1-(2-chlorophenyl)ethanol, and 3-[[4-[2-amino-1-(2-chlorophenyl)ethoxy]-6-(2,6-dimethylphenyl)pyrimidin-2-yl]sulfamoyl]benzoic Acid

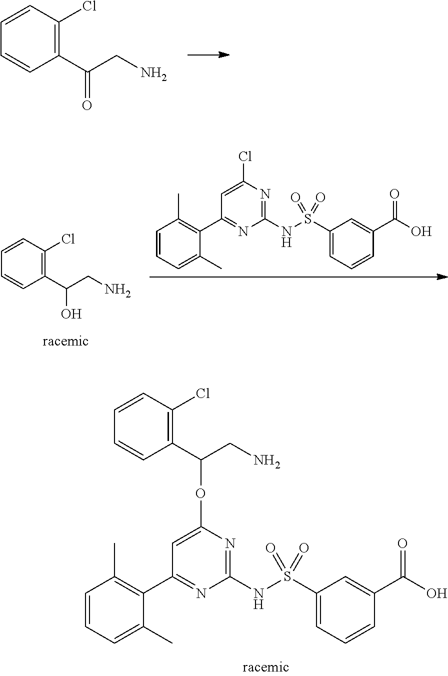 [SMILES: NCC(=O)c1ccccc1Cl ; NCC(O)c1ccccc1Cl ; Cc1cccc(C)c1-c1cc(Cl)nc(NS(=O)(=O)c2cccc(C(=O)O)c2)n1 ; Cc1cccc(C)c1-c1cc(OC(CN)c2ccccc2Cl)nc(NS(=O)(=O)c2cccc(C(=O)O)c2)n1]

Stage 1: A solution of 2-amino-1-(2-chlorophenyl)ethanone (hydrochloride salt) (100 mg, 0.4853 mmol) in MeOH (2 mL) was cooled in an ice bath and treated with sodium borohydride (19 mg, 0.5022 mmol). The mixture was stirred at RT for 1 h and evaporated in vacuo to afford crude 2-amino-1-(2-chlorophenyl)ethanol (83 mg, 100%) ESI-MS m/z calc. 171.04509, found 154.02 (M+1-18)*; Retention time: 0.25 minutes; LC method D.

Stage 2: The crude product from Stage 1 was dissolved in THF (5 mL). 3-[[4-Chloro-6-(2,6-dimethylphenyl)pyrimidin-2-yl]sulfamoyl]benzoic acid (203 mg, 0.4858 mmol) was added and the mixture was cooled in an ice bath. Sodium tert-butoxide (233 mg, 2.424 mmol) was added and the mixture was stirred at RT for 2 h. The mixture was evaporated, dissolved in MeOH, filtered and purified by preparative reverse-phase HPLC (C₁₈) to afford 3-[[4-[2-amino-1-(2-chlorophenyl)ethoxy]-6-(2,6-dimethylphenyl)pyrimidin-2-yl]sulfamoyl]benzoic Acid (hydrochloride salt) (75.6 mg, 26%); ESI-MS m/z calc. 552.1234, found 553.26 (M+1)⁺; Retention time: 1.18 minutes; LC method A.

### Step 2: 10-(2-Chlorophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 31)

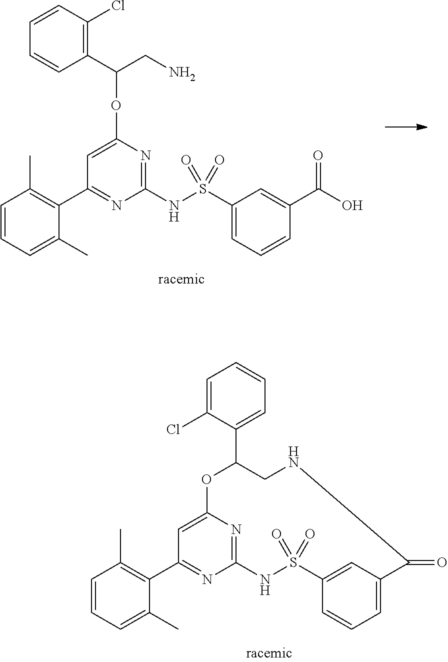 [SMILES: Cc1cccc(C)c1-c1cc(OC(CN)c2ccccc2Cl)nc(NS(=O)(=O)c2cccc(C(=O)O)c2)n1 ; Cc1cccc(C)c1-c1cc2nc(n1)NS(=O)(=O)c1cccc(c1)C(=O)NCC(c1ccccc1Cl)O2]

A solution of 3-[[4-[2-amino-1-(2-chlorophenyl)ethoxy]-6-(2,6-dimethylphenyl)pyrimidin-2-yl]sulfamoyl]benzoic acid (hydrochloride salt) (75 mg, 0.1272 mmol) and HATU (73 mg, 0.1920 mmol) in DMF (3 mL) was cooled in an ice bath. DIPEA (70 μL, 0.4019 mmol) was added and the mixture was stirred at RT for 1 h, filtered and purified by preparative reverse-phase HPLC (C₁₈) to afford 10-(2-chlorophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (33.8 mg, 49%)¹H NMR (400 MHz, DMSO-d₆) δ 8.71 (s, 1H), 8.03-7.88 (m, 2H), 7.81-7.64 (m, 3H), 7.61 (d, J 9.3 Hz, 1H), 7.55-7.46 (m, 2H), 7.25 (t, J 7.3 Hz, 1H), 7.11 (d, J=7.6 Hz, 2H), 7.01 (dd, J=10.5, 4.3 Hz, 1H), 6.34 (s, 1H), 3.52-3.37 (m, 2H), 2.04 (s, 6H). ESI-MS m/z calc. 534.11285, found 535.26 (M+1)⁺; Retention time: 1.52 minutes; LC method A.

### Example 37: Preparation of Compound 32

### Step 2: 2-Amino-1-(3-chlorophenyl)ethanol, and 3-[[4-[2-amino-1-(3-chlorophenyl)ethoxy]-6-(2,6-dimethylphenyl)pyrimidin-2-yl]sulfamoyl]benzoic Acid

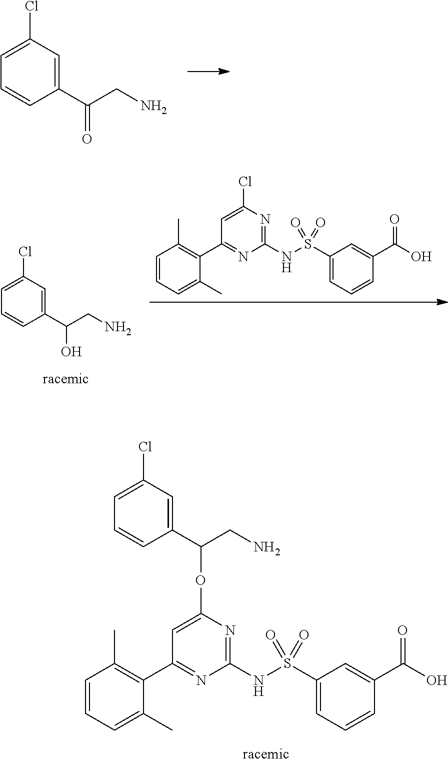 [SMILES: NCC(=O)c1cccc(Cl)c1 ; NCC(O)c1cccc(Cl)c1 ; Cc1cccc(C)c1-c1cc(Cl)nc(NS(=O)(=O)c2cccc(C(=O)O)c2)n1 ; Cc1cccc(C)c1-c1cc(OC(CN)c2cccc(Cl)c2)nc(NS(=O)(=O)c2cccc(C(=O)O)c2)n1]

Stage 1: A solution of 2-amino-1-(3-chlorophenyl)ethanone (hydrochloride salt) (80 mg, 0.3882 mmol) in MeOH (3 mL) was cooled in an ice bath and treated with sodium borohydride (15 mg, 0.3965 mmol). The mixture was stirred at RT overnight and evaporated in vacuo to afford crude 2-amino-1-(3-chlorophenyl)ethanol (66 mg, 99%) ESI-MS m/z calc. 171.04509, found 154.05 (M+1)⁺; Retention time: 1.26 minutes; LC method D.

Stage 2: The crude product from Stage 1 was dissolved in THF (4 mL). 3-[[4-Chloro-6-(2,6-dimethylphenyl)pyrimidin-2-yl]sulfamoyl]benzoic acid (160 mg, 0.3829 mmol) was added and the mixture was cooled in an ice bath. Sodium tert-butoxide (190 mg, 1.977 mmol) was added and the mixture was stirred at RT for 2 h. The mixture was evaporated, dissolved in MeOH, filtered and purified by preparative reverse phase HPLC (C₁₈) to afford 3-[[4-[2-amino-1-(3-chlorophenyl)ethoxy]-6-(2,6-dimethylphenyl)pyrimidin-2-yl]sulfamoyl]benzoic Acid (hydrochloride salt) (98.4 mg, 43%) ESI-MS m/z calc. 552.1234, found 553.26 (M+1)⁺; Retention time: 1.23 minutes; LC method D.

### Step 3: 10-(3-Chlorophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 32)

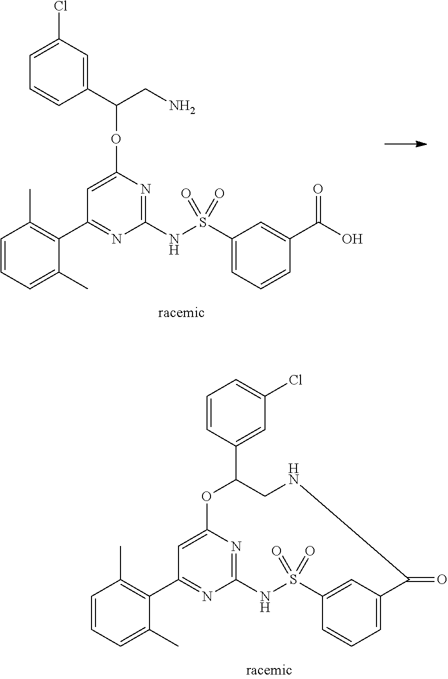 [SMILES: Cc1cccc(C)c1-c1cc(OC(CN)c2cccc(Cl)c2)nc(NS(=O)(=O)c2cccc(C(=O)O)c2)n1 ; Cc1cccc(C)c1-c1cc2nc(n1)NS(=O)(=O)c1cccc(c1)C(=O)NCC(c1cccc(Cl)c1)O2]

A solution of 3-[[4-[2-amino-1-(3-chlorophenyl)ethoxy]-6-(2,6-dimethylphenyl)pyrimidin-2-yl]sulfamoyl]benzoic acid (hydrochloride salt) (98 mg, 0.1662 mmol) and HATU (95 mg, 0.2498 mmol) in DMF (4 mL) was cooled in an ice bath. DIPEA (88 μL, 0.5052 mmol) was added and the mixture was stirred at RT for 1 h, filtered and purified by preparative reverse phase HPLC (C₁₈) to afford 10-(3-chlorophenyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (24 mg, 27%); ¹H NMR (400 MHz, DMSO-d₆) δ 8.70 (s, 1H), 7.96 (s, 1H), 7.84-7.57 (m, 5H), 7.52 (d, J 6.4 Hz, 2H), 7.25 (t, J 7.1 Hz, 1H), 7.11 (d, J 7.5 Hz, 2H), 6.45-6.17 (m, 2H), 3.60-3.45 (m, 1H), 2.05 (s, 6H). Note: One aliphatic proton overlapped with water. ESI-MS m/z calc. 534.11285, found 535.26 (M+1)⁺; Retention time: 1.63 minutes; LC method A.

### Example 38: Preparation of Compound 33

### Step 1: 4-(2-Amino-1-hydroxy-ethyl)phenol

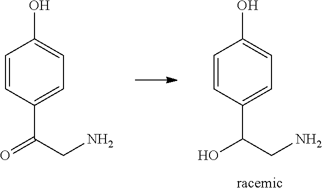 [SMILES: NCC(=O)c1ccc(O)cc1 ; NCC(O)c1ccc(O)cc1]

To a solution of 2-amino-1-(4-hydroxyphenyl)ethanone (hydrochloride salt) (1.05 g, 5.596 mmol) in MeOH (20 mL) at 0° C. was added sodium borohydride (217 mg, 5.736 mmol). The reaction was stirred for 2 h, allowing to warm to rt. The solvents were evaporated in vacuo to give crude 4-(2-amino-1-hydroxy-ethyl)phenol (hydrochloride salt) (1.0 g, 94%) ESI-MS m z calc. 153.07898, found 154.0 (M+1)⁺; Retention time: 0.1 minutes; LC method D.

### Step 2: 2-Amino-1-[4-[[1-(trifluoromethyl)cyclopropyl]methoxy]phenyl]ethanol

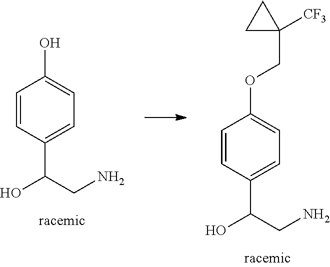 [SMILES: NCC(O)c1ccc(O)cc1 ; NCC(O)c1ccc(OCC2(C(F)(F)F)CC2)cc1]

To a solution of 1-(bromomethyl)-1-(trifluoromethyl)cyclopropane (426 mg, 2.099 mmol) and 4-(2-amino-1-hydroxy-ethyl)phenol (hydrochloride salt) (379 mg, 1.999 mmol) in DMF (1 mL) was added NaH (170 mg of 60% w/w, 4.250 mmol) at 0° C. The reaction mixture was stirred for 18 h at rt, filtered and purified by preparative reverse-phase HPLC (C₁₈) to give 2-amino-1-[4-[[1-(trifluoromethyl)cyclopropyl]methoxy]phenyl]ethanol (hydrochloride salt) (60 mg, 10%)¹H NMR (400 MHz, acetone-d₆) δ 7.29-7.23 (m, 2H), 6.94-6.86 (m, 2H), 4.77 (t, J 6.8 Hz, 1H), 4.13 (s, 2H), 3.48 (dd, J 11.8, 6.4 Hz, 1H), 2.80 (dd, J 11.8, 7.2 Hz, 1H), 1.16-1.09 (m, 2H), 1.05-0.97 (m, 2H). ESI-MS m/z calc. 275.1133, found 276.2 (M+1)⁺; Retention time: 0.98 minutes; LC method A.

### Step 3: 3-[[4-[2-Amino-1-[4-[[1-(trifluoromethyl)cyclopropyl]methoxy]phenyl]ethoxy]-6-(2,6-dimethylphenyl)pyrimidin-2-yl]sulfamoyl]benzoic acid

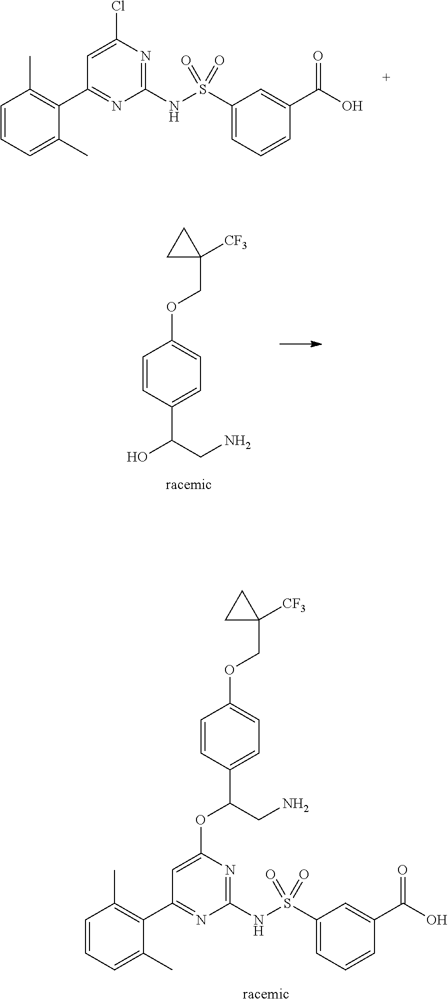 [SMILES: Cc1cccc(C)c1-c1cc(Cl)nc(NS(=O)(=O)c2cccc(C(=O)O)c2)n1 ; NCC(O)c1ccc(OCC2(C(F)(F)F)CC2)cc1 ; Cc1cccc(C)c1-c1cc(OC(CN)c2ccc(OCC3(C(F)(F)F)CC3)cc2)nc(NS(=O)(=O)c2cccc(C(=O)O)c2)n1]

To a solution of 2-amino-1-[4-[[1-(trifluoromethyl)cyclopropyl]methoxy]phenyl]ethanol (hydrochloride salt) (60 mg, 0.1925 mmol) and 3-[[4-chloro-6-(2,6-dimethylphenyl)pyrimidin-2-yl]sulfamoyl]benzoic acid (67 mg, 0.1603 mmol) in DMF (0.8 mL) was added NaH (39 mg of 60% w/w, 0.9751 mmol) portion-wise at 0° C. over 1 h. The reaction mixture was allowed to warm to rt and stirred for 18 h, after which it was filtered and purified by preparative reverse phase HPLC (C₁₈) to give 3-[[4-[2-amino-1-[4-[[1-(trifluoromethyl)cyclopropyl]methoxy]phenyl]ethoxy]-6-(2,6-dimethylphenyl)pyrimidin-2-yl]sulfamoyl]benzoic acid (hydrochloride salt) (27 mg, 24%) ESI-MS m/z calc. 656.19165, found 657.29 (M+1)⁺; Retention time: 1.44 minutes; LC method A.

### Step 4: 6-(2,6-Dimethylphenyl)-2,2-dioxo-10-[4-[[1-(trifluoromethyl)cyclopropyl]methoxy]phenyl]-9-oxa-2λ⁶-thia-3,5,12,19-tetrazatricyclo[12.3.1.14,8]nonadeca-1(18),4(19),5,7,14,16-hexaen-13-one (Compound 33)

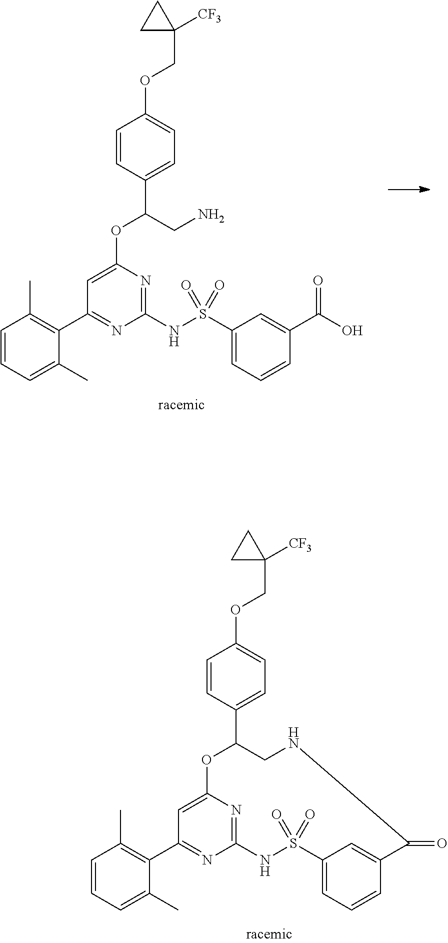 [SMILES: Cc1cccc(C)c1-c1cc(OC(CN)c2ccc(OCC3(C(F)(F)F)CC3)cc2)nc(NS(=O)(=O)c2cccc(C(=O)O)c2)n1 ; Cc1cccc(C)c1-c1cc2nc(n1)NS(=O)(=O)c1cccc(c1)C(=O)NCC(c1ccc(OCC3(C(F)(F)F)CC3)cc1)O2]

To a solution of 3-[[4-[2-amino-1-[4-[[1-(trifluoromethyl)cyclopropyl]methoxy]phenyl]ethoxy]-6-(2,6-dimethylphenyl)pyrimidin-2-yl]sulfamoyl]benzoic acid (hydrochloride salt) (27 mg, 0.03895 mmol) in DMF (0.8 mL) was added HATU (20 mg, 0.05260 mmol), and the reaction mixture was stirred for 1 min. DIPEA (30 μL, 0.1722 mmol) was added, and the reaction was stirred for 3 days at rt, after which it was filtered and purified by preparative reverse-phase HPLC (C₁₈) to yield 6-(2,6-dimethylphenyl)-2,2-dioxo-10-[4-[[1-(trifluoromethyl)cyclopropyl]methoxy]phenyl]-9-oxa-2λ⁶-thia-3,5,12,19-tetrazatricyclo[12.3.1.14,8]nonadeca-1(18),4(19),5,7,14,16-hexaen-13-one (2.5 mg, 10%); ESI-MS m/z calc. 638.1811, found 639.1 (M+1)⁺; Retention time: 1.8 minutes; LC method A.

### Example 39: Preparation of Compound 34

### Step 1: 3-[[4-(2-Amino-1-tetrahydropyran-4-yl-ethoxy)-6-(2,6-dimethylphenyl)pyrimidin-2-yl]sulfamoyl]benzoic Acid

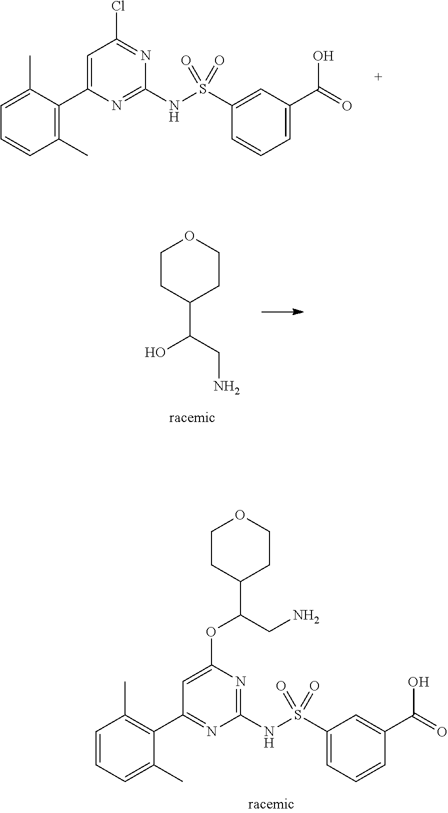 [SMILES: Cc1cccc(C)c1-c1cc(Cl)nc(NS(=O)(=O)c2cccc(C(=O)O)c2)n1 ; NCC(O)C1CCOCC1 ; Cc1cccc(C)c1-c1cc(OC(CN)C2CCOCC2)nc(NS(=O)(=O)c2cccc(C(=O)O)c2)n1]

In a 20 mL flask, 3-[[4-chloro-6-(2,6-dimethylphenyl)pyrimidin-2-yl]sulfamoyl]benzoic acid (265 mg, 0.6342 mmol), 2-amino-1-tetrahydropyran-4-yl-ethanol (hydrochloride salt) (115 mg, 0.6330 mmol) and THE (5 mL) were mixed and cooled in an ice-bath at 0° C., to which KOtBu (427 mg, 3.805 mmol) was added. This mixture was stirred 30 min at 0° C. The mixture was immediately quenched with a small amount of methanol dropwise, then the mixture was filtered and purified by reverse-phase preparative HPLC (C₁₈) to give a white solid, 3-[[4-(2-amino-1-tetrahydropyran-4-yl-ethoxy)-6-(2,6-dimethylphenyl)pyrimidin-2-yl]sulfamoyl]benzoic acid (hydrochloride salt) (159.4 mg, 45%) ESI-MS m/z calc. 526.1886, found 527.2 (M+1)⁺; Retention time: 0.89 minutes; LC method A.

### Step 2: 6-(2,6-Dimethylphenyl)-2,2-dioxo-10-tetrahydropyran-4-yl-9-oxa-2λ⁶-thia-3,5,12,19-tetrazatricyclo[12.3.1.14,8]nonadeca-1(18),4(19),5,7,14,16-hexaen-13-one (Compound 34)

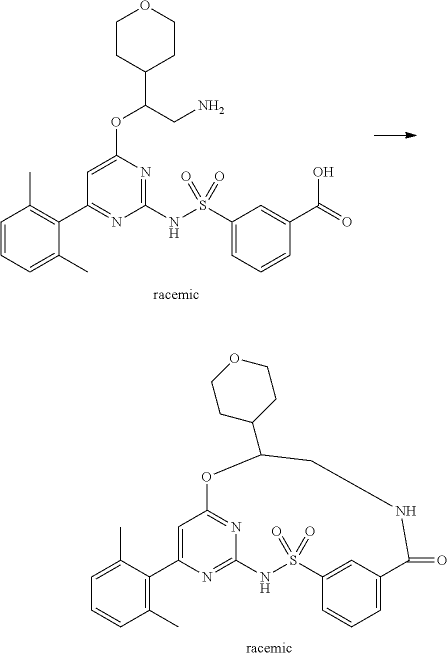 [SMILES: Cc1cccc(C)c1-c1cc(OC(CN)C2CCOCC2)nc(NS(=O)(=O)c2cccc(C(=O)O)c2)n1 ; Cc1cccc(C)c1-c1cc2nc(n1)NS(=O)(=O)c1cccc(c1)C(=O)NCC(C1CCOCC1)O2]

In a 20-mL vial, 3-[[4-(2-amino-1-tetrahydropyran-4-yl-ethoxy)-6-(2,6-dimethylphenyl)pyrimidin-2-yl]sulfamoyl]benzoic acid (hydrochloride salt) (149 mg, 0.2646 mmol) was dissolved in DMF (4.0 mL), to which DIPEA (400 μL, 2.296 mmol) and HATU (158 mg, 0.4155 mmol) were added. After stirring at room temperature for 15 min, the mixture was quenched with water and filtered. This mixture was purified by reverse-phase preparative HPLC (C₁₈) to afford an off-white solid, 6-(2,6-dimethylphenyl)-2,2-dioxo-10-tetrahydropyran-4-yl-9-oxa-2λ⁶-thia-3,5,12,19-tetrazatricyclo[12.3.1.14,8]nonadeca-1(18),4(19),5,7,14,16-hexaen-13-one (34.5 mg, 25%); ¹H NMR (400 MHz, DMSO-d₆) δ 12.94 (s, 1H), 8.50 (s, 1H), 8.33 (dd, J 9.5, 5.3 Hz, 1H), 7.92 (d, J 7.1 Hz, 1H), 7.75-7.59 (m, 2H), 7.26 (t, J 7.6 Hz, 1H), 7.12 (d, J=7.7 Hz, 2H), 6.33 (s, 1H), 5.31-5.12 (m, 1H), 4.00-3.88 (m, 2H), 3.40-3.34 (m, 2H), 3.20-3.04 (m, 2H), 2.31-2.21 (m, 1H), 2.13-1.97 (m, 6H), 1.77 (d, J 12.3 Hz, 1H), 1.65-1.46 (m, 3H). ESI-MS m/z calc. 508.17804, found 509.3 (M+1)⁺; Retention time: 1.18 minutes; LC method A.

### Example 40: Preparation of Compound 35 and Compound 36

### Step 1: (2S,5R)-2-Isopropyl-3,6-dimethoxy-5-oct-7-enyl-2,5-dihydropyrazine

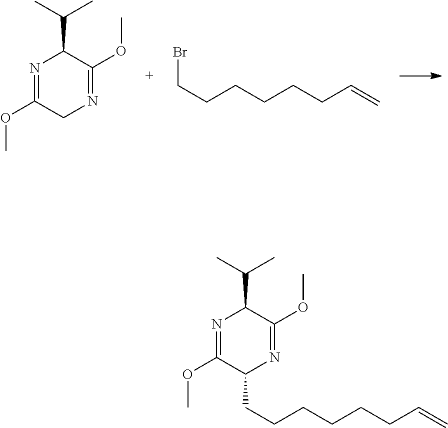

In a 100-mL round-bottomed flask, a solution of (2S)-2-isopropyl-3,6-dimethoxy-2,5-dihydropyrazine (1.3274 g, 7.205 mmol) in THE (20 mL) was cooled to −78° C. n-BuLi (3.4 mL of 2.5 M, 8.500 mmol) was added, and the resulting solution was stirred at −78° C. for 45 min. After this time, 8-brom ° C. t-1-ene (1.4 mL, 8.344 mmol) was added over 15 min, and the resulting solution was stirred at −78° C. for 5 h. Then, the cooling bath was removed, and the mixture was stirred for 16 h. It was then quenched with saturated aqueous ammonium chloride solution (40 mL) and extracted with ethyl acetate (3×40 mL). The combined organic extracts were washed with water (100 mL) and saturated aqueous sodium chloride solution (100 mL), then dried over sodium sulfate, filtered, and evaporated in vacuo. The resulting orange oil was purified by silica gel chromatography (80 g of silica) using a gradient eluent of 0 to 15% ethyl acetate in hexanes to give a slightly yellow transparent liquid, (2S,5R)-2-isopropyl-3,6-dimethoxy-5-oct-7-enyl-2,5-dihydropyrazine (1.549 g, 73%)¹H NMR (500 MHz, DMSO-d₆) [˜20:1 dr; peak listing of the major diastereomer is shown] δ 5.79 (ddt, J 16.9, 10.1, 6.7 Hz, 1H), 4.99 (d, J 17.3 Hz, 1H), 4.94 (d, J 10.2 Hz, 1H), 4.01 (dt, J 7.1, 3.9 Hz, 1H), 3.92 (t, J=3.5 Hz, 1H), 3.62 (s, 3H), 3.60 (s, 3H), 2.26-2.14 (m, 1H), 2.00 (q, J=7.1 Hz, 2H), 1.76-1.65 (m, 1H), 1.65-1.55 (m, 1H), 1.39-1.29 (m, 2H), 1.29-1.09 (m, 6H), 1.00 (d, J=6.8 Hz, 3H), 0.62 (d, J 6.8 Hz, 3H). ESI-MS m/z calc. 294.23074, found 295.2 (M+1)⁺; Retention time: 2.11 minutes (major) and 2.05 min (minor); LC method A.

### Step 2: Methyl (2R)-2-aminodec-9-enoate

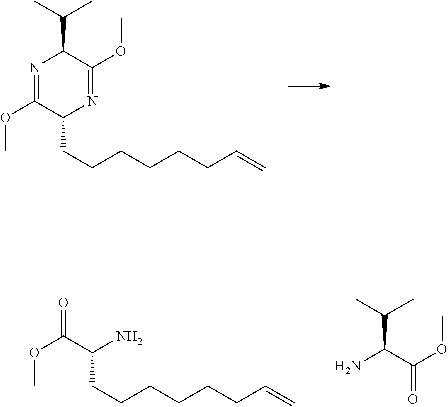

In a 250-mL round-bottomed flask, (2S,5R)-2-isopropyl-3,6-dimethoxy-5-oct-7-enyl-2,5-dihydropyrazine (1.545 g, 5.247 mmol) was dissolved in MeCN (30 mL), to which aqueous HCl (30 mL of 1.0 M, 30.00 mmol) was added. The resulting mixture was stirred at room temperature for 4 h, after which it was quenched with saturated aqueous sodium bicarbonate solution (60 mL). The mixture was extracted with dichloromethane (3×30 mL). The combined organic extracts were then dried over sodium sulfate, filtered, and evaporated in vacuo. The resulting yellow oil was purified by silica gel chromatography (40 g of silica) using a gradient eluent of 1 to 10% methanol in dichloromethane to give a yellow liquid, methyl (2R)-2-aminodec-9-enoate (0.8380 g, 80%). The stoichiometric side product (valine methyl ester) was not detected in the ¹H NMR spectrum. ¹H NMR (500 MHz, DMSO-d₆) δ 5.80 (ddt, J 17.0, 10.2, 6.7 Hz, 1H), 5.00 (d, J 17.1 Hz, 1H), 4.94 (d, J 10.2 Hz, 1H), 3.61 (s, 3H), 3.28 (dd, J=7.5, 5.7 Hz, 1H), 2.01 (q, J 7.1 Hz, 2H), 1.84-1.58 (bs, 2H), 1.58-1.49 (m, 1H), 1.47-1.37 (m, 1H), 1.37-1.30 (m, 2H), 1.30-1.19 (m, 6H) ESI-MS m/z calc. 199.15723, found 200.1 (M+1)⁺; Retention time: 1.0 minutes; LC method A.

### Step 3: (2R)-2-Aminodec-9-en-1-ol

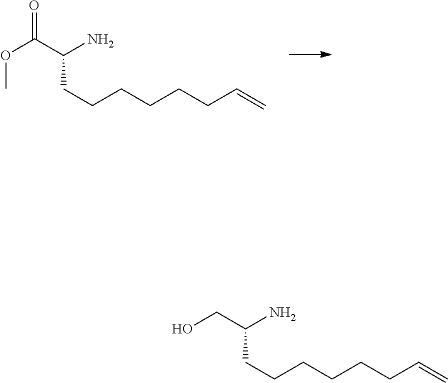

In a 100-mL round-bottomed flask, methyl (2R)-2-aminodec-9-enoate (837.5 mg, 4.202 mmol) was dissolved in MeOH (25 mL) and cooled to 0° C. Sodium borohydride (403.5 mg, 10.67 mmol) was added in one portion, upon which the ice-water bath was removed. The reaction mixture was allowed to stir at room temperature in an open flask for 5 h. Then, the reaction mixture was cooled to 0° C., and a second portion of sodium borohydride (403.5 mg, 10.67 mmol) was added. The ice-water bath was removed, and the reaction mixture was allowed to stir at room temperature in an open flask for 16 h. Then, the reaction mixture was cooled to 0° C., and a third portion of sodium borohydride (403.5 mg, 10.67 mmol) was added. The ice-water bath was removed, and the reaction mixture was allowed to stir at room temperature in an open flask for 6 h. After this time, the mixture was evaporated to dryness in vacuo. The resulting residue was dissolved in water (50 mL), which was extracted with ethyl acetate (6×50 mL). The combined organic extracts was dried over sodium sulfate, filtered, and evaporated in vacuo to give a slightly viscous, colorless oil: (2R)-2-aminodec-9-en-1-ol (714.0 mg, 99%); ESI-MS m z calc. 171.16231, found 172.0 (M+1)⁺; Retention time: 0.86 minutes; LC method A.

### Step 4: 4-Chloro-6-(2-vinylphenyl)pyrimidin-2-amine

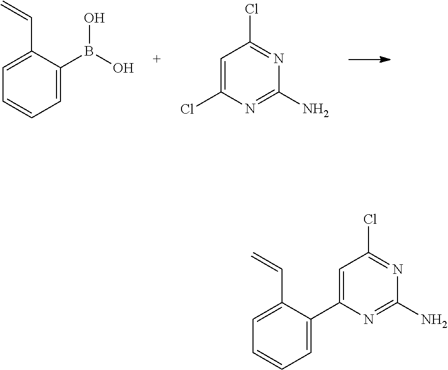 [SMILES: C=Cc1ccccc1B(O)O ; Nc1nc(Cl)cc(Cl)n1 ; C=Cc1ccccc1-c1cc(Cl)nc(N)n1]

In a 500-mL round-bottomed flask, (2-vinylphenyl)boronic acid (9.9024 g, 64.92 mmol), 4,6-dichloropyrimidin-2-amine (15.001 g, 88.73 mmol), Pd(PPh3)4 (4 g, 3.462 mmol), a solution of potassium carbonate (24 g, 173.7 mmol) in water (80 mL), as well as CH₃CN (160 mL) were mixed together. The resulting mixture was sparged with nitrogen gas for 15 min. This mixture was stirred at 90° C. (reflux) for 16.5 h. After this time, the reaction mixture was cooled to room temperature, poured into water (300 mL) and extracted with EtOAc (3×300 mL). The combined organic extracts were washed with water (400 mL) and saturated aqueous sodium chloride solution (400 mL), then dried over sodium sulfate, filtered, and evaporated in vacuo. The resulting brown foam was purified by silica gel chromatography (330 g of silica) using a gradient eluent of 1 to 30% ethyl acetate in hexanes to give a white solid, 4-chloro-6-(2-vinylphenyl)pyrimidin-2-amine (4.937 g, 33%)¹H NMR (500 MHz, DMSO-d₆) δ 7.72 (d, J 7.8 Hz, 1H), 7.47 (t, J 7.8 Hz, 2H), 7.44 (d, J 7.6 Hz, 1H), 7.39 (t, J 7.4 Hz, 1H), 7.26 (s, 2H), 6.90 (dd, J 17.5, 11.0 Hz, 1H), 6.69 (s, 1H), 5.79 (d, J 17.5 Hz, 1H), 5.31 (d, J 11.1 Hz, 1H) ESI-MS m/z calc. 231.05632, found 232.0 (M+1)⁺; Retention time: 1.44 minutes; LC method A.

### Step 5: Methyl 3-[[4-chloro-6-(2-vinylphenyl)pyrimidin-2-yl]sulfamoyl]benzoate

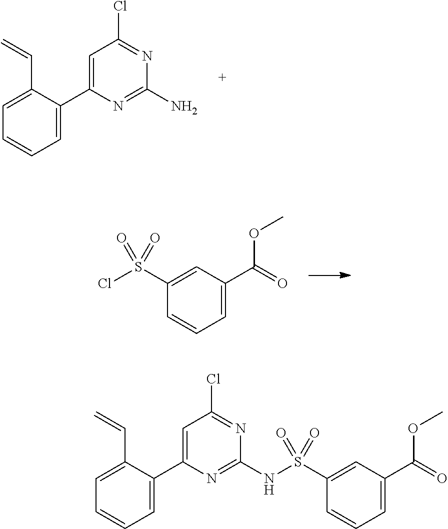 [SMILES: C=Cc1ccccc1-c1cc(Cl)nc(N)n1 ; COC(=O)c1cccc(S(=O)(=O)Cl)c1 ; C=Cc1ccccc1-c1cc(Cl)nc(NS(=O)(=O)c2cccc(C(=O)OC)c2)n1]

In a 250-mL round-bottomed flask, 4-chloro-6-(2-vinylphenyl)pyrimidin-2-amine (4.937 g, 21.31 mmol) was dissolved in THE (50 mL) and cooled to 0° C. Solid methyl 3-chlorosulfonylbenzoate (7.54 g, 32.13 mmol) was added in one portion, followed by slow addition of a heptanes solution of lithium tert-amoxide (20 mL of 40% w/w, 62.07 mmol) over 5 min. The resulting orange transparent solution was warmed to room temperature while stirring over 2.5 h. After this time, 1 N HCl solution (60 mL) was added, and the resulting mixture was extracted with ethyl acetate (3×100 mL). The combined organic extracts was washed with water (200 mL) and saturated aqueous sodium chloride solution (200 mL), then dried over sodium sulfate, filtered, and evaporated in vacuo. The resulting orange oil was purified by silica gel chromatography (330 g of silica) using a gradient eluent of 1 to 70% ethyl acetate in hexanes to give a white foam, methyl 3-[[4-chloro-6-(2-vinylphenyl)pyrimidin-2-yl]sulfamoyl]benzoate (7.805 g, 80%)¹H NMR (500 MHz, DMSO-d₆) δ 12.49 (s, 1H), 8.55 (s, 1H), 8.20 (t, J 8.1 Hz, 2H), 7.74 (t, J 7.8 Hz, 1H), 7.72 (d, J 7.8 Hz, 1H), 7.52 (t, J 7.6 Hz, 1H), 7.38 (t, J 7.5 Hz, 1H), 7.30 (d, J=7.4 Hz, 1H), 7.29 (s, 1H), 6.85 (dd, J=17.4, 11.0 Hz, 1H), 5.78 (d, J=17.3 Hz, 1H), 5.25 (d, J=11.0 Hz, 1H), 3.84 (s, 3H) ESI-MS m/z calc. 429.055, found 430.1 (M+1)⁺; Retention time: 1.81 minutes; LC method A.

### Step 6: 3-[[4-Chloro-6-(2-vinylphenyl)pyrimidin-2-yl]sulfamoyl]benzoic Acid

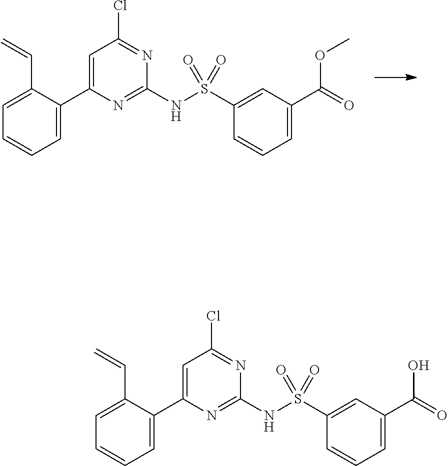 [SMILES: C=Cc1ccccc1-c1cc(Cl)nc(NS(=O)(=O)c2cccc(C(=O)OC)c2)n1 ; C=Cc1ccccc1-c1cc(Cl)nc(NS(=O)(=O)c2cccc(C(=O)O)c2)n1]

In a 250-mL round-bottomed flask, methyl 3-[[4-chloro-6-(2-vinylphenyl)pyrimidin-2-yl]sulfamoyl]benzoate (7.805 g, 17.07 mmol) was dissolved in THE (40 mL), to which aqueous NaOH (40 mL of 1.0 M, 40.00 mmol) was added. The mixture was stirred at room temperature for 1 h, after which a second portion of aqueous NaOH (40 mL of 1.0 M, 40.00 mmol) was added. The resulting mixture was stirred at room temperature for 1 h, after which 1 N HCl solution (120 mL) was added, and the resulting mixture was extracted with ethyl acetate (3×100 mL). The combined organic extracts was washed with water (100 mL) and saturated aqueous sodium chloride solution (100 mL), then dried over sodium sulfate, filtered, and evaporated in vacuo to give a white powder: 3-[[4-chloro-6-(2-vinylphenyl)pyrimidin-2-yl]sulfamoyl]benzoic acid (6.744 g, 95%); ¹H NMR (500 MHz, DMSO-d₆) δ 13.94-13.01 (bs, 1H), 12.90-12.15 (bs, 1H), 8.53 (d, J=1.9 Hz, 1H), 8.20 (dd, J=7.9, 1.8 Hz, 2H), 7.72 (t, J 7.8 Hz, 1H), 7.71 (d, J=7.8 Hz, 1H), 7.51 (t, J=7.6 Hz, 1H), 7.38 (t, J=7.5 Hz, 1H), 7.30 (d, J 7.7 Hz, 1H), 7.28 (s, 1H), 6.84 (dd, J 17.3, 11.0 Hz, 1H), 5.77 (d, J 17.3 Hz, 1H), 5.26 (d, J=11.1 Hz, 1H); ESI-MS m/z calc. 415.03937, found 416.1 (M+1)⁺; Retention time: 1.55 minutes; LC method A.

### Step 7: (11R)-11-Oct-7-enyl-2,2-dioxo-6-(2-vinylphenyl)-9-oxa-2λ⁶-thia-3,5,12,19-tetrazatricyclo[12.3.1.14,8]nonadeca-1(18),4,6,8(19),14,16-hexaen-13-one (Compound 35)

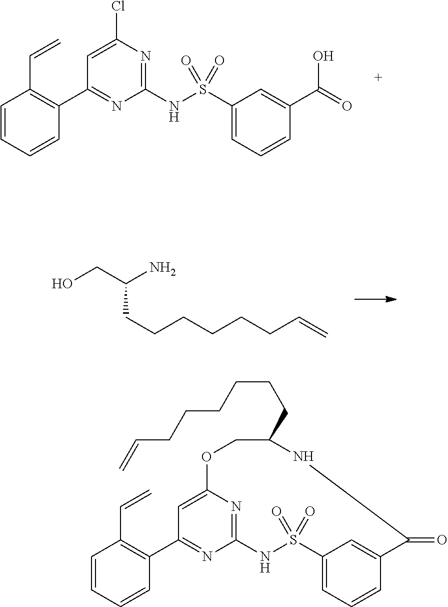 [SMILES: C=Cc1ccccc1-c1cc(Cl)nc(NS(=O)(=O)c2cccc(C(=O)O)c2)n1 ; C=CCCCCCC[C@@H](N)CO ; C=CCCCCCC[C@@H]1COc2cc(-c3ccccc3C=C)nc(n2)NS(=O)(=O)c2cccc(c2)C(=O)N1]

In a 50-mL round-bottomed flask, (2R)-2-aminodec-9-en-1-ol (0.712 g, 4.157 mmol) was dissolved in THE (10 mL), to which 3-[[4-chloro-6-(2-vinylphenyl)pyrimidin-2-yl]sulfamoyl]benzoic acid (1.6947 g, 4.075 mmol) and NaOtBu (1.9425 g, 20.21 mmol) were added. The resulting mixture was stirred at room temperature for 1 h. In a separate 500-mL round-bottomed flask, a solution of HATU (3.76 g, 9.889 mmol) in DMF (200 mL) was prepared and cooled to 0° C. Then, the reaction mixture in the 50-mL flask was added slowly (over 30 min) into this 500-mL flask. The resulting solution was stirred at 0° C. for 10 min, after which it was quenched by pouring onto 1 N HCl solution (200 mL). The mixture was extracted with ethyl acetate (3×200 mL). The combined organic extracts were washed with water (300 mL) and saturated aqueous sodium chloride solution (300 mL), then dried over sodium sulfate, filtered, and evaporated in vacuo. The resulting yellow oil (contains some DMF) was purified by silica gel chromatography (80 g of silica) using a gradient eluent of 1 to 90% ethyl acetate in hexanes to give a white solid, (11R)-11-oct-7-enyl-2,2-dioxo-6-(2-vinylphenyl)-9-oxa-2λ⁶-thia-3,5,12,19-tetrazatricyclo[12.3.1.14,8]nonadeca-1(18),4,6,8(19),14,16-hexaen-13-one (758.0 mg, 35%)¹H NMR (500 MHz, DMSO-d₆) δ 8.52 (s, 1H), 7.95 (s, 1H), 7.89 (d, J 9.9 Hz, 1H), 7.76-7.65 (m, 3H), 7.54-7.46 (m, 1H), 7.42-7.35 (m, 2H), 6.77 (dd, J 17.4, 11.3 Hz, 1H), 6.42 (s, 1H), 5.79 (d, J 17.4 Hz, 1H), 5.68 (ddt, J 16.9, 10.2, 6.6 Hz, 1H), 5.30 (d, J 11.0 Hz, 1H), 5.15 (dd, J 10.6, 3.1 Hz, 1H), 4.95-4.83 (m, 2H), 3.87 (t, J 11.2 Hz, 1H), 3.23 (q, J 10.8 Hz, 1H), 1.87-1.77 (m, 2H), 1.59-1.40 (m, 2H), 1.32-1.24 (m, 1H), 1.22-1.11 (m, 2H), 1.10-1.02 (m, 2H), 1.01-0.89 (m, 3H) ESI-MS m/z calc. 532.2144, found 533.3 (M+1)⁺; Retention time: 1.86 minutes; LC method A.

### Step 8: (11R)-3-(methoxymethyl)-11-oct-7-enyl-2,2-dioxo-6-(2-vinylphenyl)-9-oxa-2λ⁶-thia-3,5,12,19-tetrazatricyclo[12.3.1.14,8]nonadeca-1(17),4(19),5,7,14(18),15-hexaen-13-one

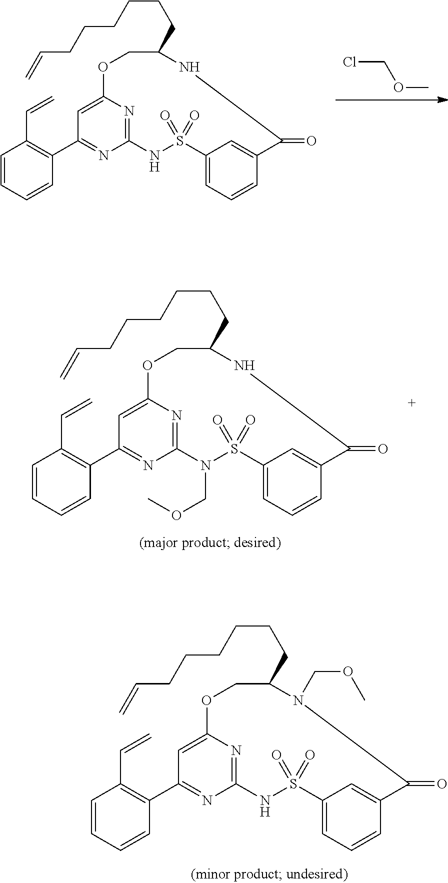

In a 100-mL round-bottomed flask, (11R)-11-oct-7-enyl-2,2-dioxo-6-(2-vinylphenyl)-9-oxa-2λ⁶-thia-3,5,12,19-tetrazatricyclo[12.3.1.14,8]nonadeca-1(18),4,6,8(19),14,16-hexaen-13-one (680.0 mg, 1.277 mmol) was dissolved in DCM (20 mL) and cooled to 0° C. Then, DIPEA (230 μL, 1.320 mmol) was added, followed by chloro(methoxy)methane (100 μL, 1.317 mmol). The resulting mixture was stirred at 0° C. for 5 min, after which it was evaporated in vacuo. Purification by silica gel chromatography (40 g of silica) using a gradient eluent of 1 to 100% ethyl acetate in hexanes gave two products with the same molecular weight: Desired product, major product, less polar material, white foam, (11R)-3-(methoxymethyl)-11-oct-7-enyl-2,2-dioxo-6-(2-vinylphenyl)-9-oxa-2λ⁶-thia-3,5,12,19-tetrazatricyclo[12.3.1.14,8]nonadeca-1(17),4(19),5,7,14(18),15-hexaen-13-one (475.0 mg, 64%); ESI-MS m/z calc. 576.24066, found 577.5 (M+1)⁺; Retention time: 2.25 minutes; LC method A. ¹H NMR (500 MHz, DMSO-d₆) δ 8.64 (s, 1H), 8.12 (d, J=7.7 Hz, 1H), 7.95 (d, J=9.7 Hz, 1H), 7.82 (d, J=7.5 Hz, 1H), 7.78 (t, J 7.6 Hz, 1H), 7.71 (d, J 7.8 Hz, 1H), 7.52 (d, J 7.5 Hz, 1H), 7.49 (d, J 7.8 Hz, 1H), 7.41 (t, J 7.5 Hz, 1H), 6.87 (dd, J 17.4, 11.0 Hz, 1H), 6.79 (s, 1H), 5.77 (d, J 17.6 Hz, 1H), 5.74 (d, J 10.9 Hz, 1H), 5.66 (ddt, J 16.9, 10.1, 6.5 Hz, 1H), 5.55 (d, J 10.7 Hz, 1H), 5.28 (d, J 11.0 Hz, 1H), 5.10 (dd, J 11.3, 3.9 Hz, 1H), 4.87 (d, J 16.1 Hz, 1H), 4.85 (d, J 8.9 Hz, 1H), 3.90 (t, J 11.3 Hz, 1H), 3.28 (q, J 10.9, 10.4 Hz, 1H), 3.11 (s, 3H), 1.81 (q, J 6.9 Hz, 2H), 1.59-1.42 (m, 2H), 1.34-1.21 (m, 1H), 1.20-1.06 (m, 2H), 1.06-0.96 (m, 3H), 0.96-0.79 (m, 2H), and a side product, minor product, more polar material, white solid, (11R)-12-(methoxymethyl)-11-oct-7-enyl-2,2-dioxo-6-(2-vinylphenyl)-9-oxa-2λ⁶-thia-3,5,12,19-tetrazatricyclo[12.3.1.14,8]nonadeca-1(18),4,6,8(19),14,16-hexaen-13-one (154.5 mg, 21%) ESI-MS m/z calc. 576.24066, found 577.5 (M+1)⁺; Retention time: 1.89 minutes; LC method A.

### Step 9: (10E,18R)-27-(Methoxymethyl)-30-oxa-26λ⁶-thia-19,27,29,32-tetraazapentacyclo[16.11.2.13,28.121,25.04,9]tritriaconta-1,3(32),4(9),5,7,10,21(33),22,24,28-decaene-20,26,26-trione and (10E,18R)-30-oxa-26)⁶-thia-19,27,29,32-tetraazapentacyclo[16.11.2.13,28.121,25.04,9]tritriaconta-1,3(32),4(9),5,7,10,21(33),22,24,28-decaene-20,26,26-trione (Compound 36)

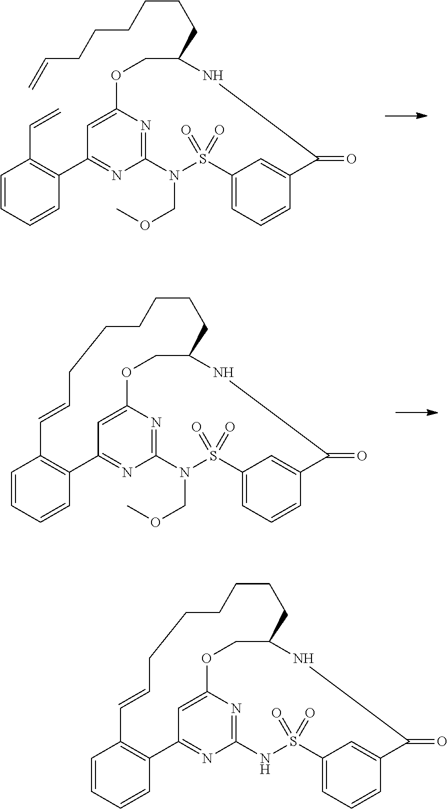 [SMILES: C=CCCCCCC[C@@H]1COc2cc(-c3ccccc3C=C)nc(n2)N(COC)S(=O)(=O)c2cccc(c2)C(=O)N1 ; COCN1c2nc3cc(n2)-c2ccccc2/C=C/CCCCCC[C@H](CO3)NC(=O)c2cccc(c2)S1(=O)=O ; O=C1N[C@@H]2CCCCCC/C=C/c3ccccc3-c3cc(nc(n3)NS(=O)(=O)c3cccc1c3)OC2]

Stage 1: In a 100-mL round-bottomed flask, (11R)-3-(methoxymethyl)-11-oct-7-enyl-2,2-dioxo-6-(2-vinylphenyl)-9-oxa-2λ⁶-thia-3,5,12,19-tetrazatricyclo[12.3.1.14,8]nonadeca-1(17),4(19),5,7,14(18),15-hexaen-13-one (472 mg, 0.8184 mmol) was dissolved in anhydrous toluene (50 mL), to which Grubbs II Catalyst (31.4 mg, 0.03699 mmol) was added. This mixture was sparged with a balloon of nitrogen gas for 10 min. It was then stirred at 100° C. for 1.5 h, after which it was cooled to room temperature, and evaporated in vacuo. The resulting brown oil was purified by silica gel chromatography (24 g of silica) using a gradient eluent of 1 to 80% ethyl acetate in hexanes to give (10E,18R)-27-(methoxymethyl)-30-oxa-26λ⁶-thia-19,27,29,32-tetraazapentacyclo[16.11.2.13,28.121,25.04,9]tritriaconta-1,3(32),4(9),5,7,10,21(33),22,24,28-decaene-20,26,26-trione (360.6 mg, 80%) ESI-MS m/z calc. 548.20935, found 549.4 (M+1)⁺; Retention time: 2.02 minutes; LC method A.

Stage 2: In a 20-mL vial, the product from Stage 1 was mixed with a dioxane solution of HCl (8.0 mL of 4.0 M, 32.00 mmol) and vigorously stirred at room temperature for 10 min. This mixture was evaporated in vacuo to give a white solid, (10E,18R)-30-oxa-26λ⁶-thia-19,27,29,32-tetraazapentacyclo[16.11.2.13,28.121,25.04,9]tritriaconta-1,3(32),4(9),5,7,10,21(33),22,24,28-decaene-20,26,26-trione (388.6 mg, 94%) (yield over 2 steps; this is an impure compound considering the apparent increase in yield). A portion (21 mg) of this compound was dissolved in 1:1 MeOH:DMSO (800 μL), filtered, and purified by reverse-phase preparative chromatography using a C₁₈ column and a gradient eluent of 1 to 70% acetonitrile in water containing 5 mM hydrochloric acid to give 8.3 mg of pure product. ¹H NMR (500 MHz, DMSO-d₆) δ 13.51-11.79 (broad d, 1H), 8.50 (s, 1H), 7.95 (s, 1H), 7.85 (d, J 9.8 Hz, 1H), 7.69 (s, 2H), 7.44 (t, J 7.2 Hz, 1H), 7.37 (d, J 7.2 Hz, 2H), 7.33 (t, J 7.2 Hz, 1H), 6.33 (s, 1H), 6.17 (d, J 16.5 Hz, 1H), 5.54 (dt, J 16.2, 6.1 Hz, 1H), 5.17 (dd, J 10.7, 3.9 Hz, 1H), 3.89 (t, J 11.1 Hz, 1H), 3.37-3.19 (m, 1H, overlapping with water peak), 2.17-2.05 (m, 1H, overlapping with MeCN impurity), 1.97-1.84 (m, 1H), 1.69-1.53 (m, 1H), 1.50-1.38 (m, 1H), 1.37-1.21 (m, 2H), 1.18-1.07 (m, 1H), 1.04-0.92 (m, 1H), 0.85-0.66 (m, 2H), 0.66-0.55 (m, 1H), 0.43-0.23 (m, 1H). ESI-MS m/z calc. 504.18314, found 505.2 (M+1)⁺; Retention time: 1.49 minutes; LC method A.

### Example 41: Preparation of Compound 37

### Step 1: (18R)-30-Oxa-26λ⁶-thia-19,27,29,32-tetraazapentacyclo[16.11.2.13,28.121,25.04,9]tritriaconta-1,3(32),4(9),5,7,21(33),22,24,28-nonaene-20,26,26-trione (Compound 37)

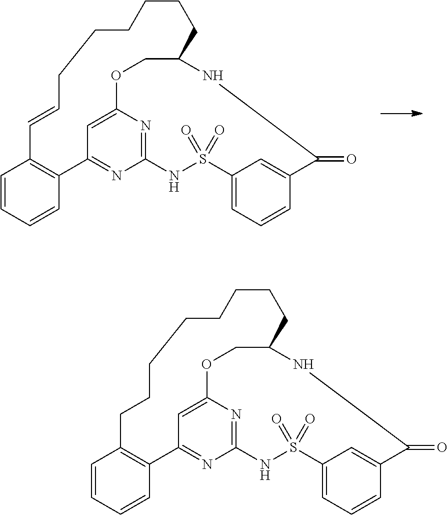 [SMILES: O=C1N[C@@H]2CCCCCC/C=C/c3ccccc3-c3cc(nc(n3)NS(=O)(=O)c3cccc1c3)OC2 ; O=C1N[C@@H]2CCCCCCCCc3ccccc3-c3cc(nc(n3)NS(=O)(=O)c3cccc1c3)OC2]

In a 3-mL microwave vial, (10E,18R)-30-oxa-26λ⁶-thia-19,27,29,32-tetraazapentacyclo[16.11.2.13,28.121,25.04,9]tritriaconta-1,3(32),4(9),5,7,10,21(33),22,24,28-decaene-20,26,26-trione (29.3 mg, 0.05807 mmol) was dissolved in EtOH (500 μL), and sparged with a balloon of nitrogen gas for 5 min. Pd(OH)₂/C (5 mg of 10% w/w, 0.003560 mmol) was added, and the resulting mixture was stirred under a balloon of hydrogen at 70° C. for 10 min. The reaction mixture was then cooled to room temperature, filtered, and purified by reverse-phase preparative chromatography using a C₁₈ column and a gradient eluent of 1 to 70% acetonitrile in water containing 5 mM hydrochloric acid to give (18R)-30-oxa-26λ⁶-thia-19,27,29,32-tetraazapentacyclo[16.11.2.13,28.121,25.04,9]tritriaconta-1,3(32),4(9),5,7,21(33),22,24,28-nonaene-20,26,26-trione (9.2 mg, 31%)¹H NMR (500 MHz, DMSO-d₆) δ 13.56-11.64 (broad d, 1H), 8.49 (s, 1H), 7.97 (s, 1H), 7.85 (d, J 9.8 Hz, 1H), 7.70 (s, 2H), 7.41 (s, 1H), 7.35-7.26 (m, 3H), 6.50 (s, 1H), 5.13 (dd, J 10.8, 3.5 Hz, 1H), 3.92 (t, J=11.1 Hz, 1H), 3.46-3.35 (m, 1H), 2.78-2.18 (broad m, 1H), 2.44-2.33 (m, 1H), 1.64-1.53 (m, 1H), 1.48-1.37 (m, 1H), 1.29-1.12 (m, 4H), 1.07-0.95 (m, 1H), 0.95-0.76 (m, 3H), 0.74-0.61 (m, 1H), 0.61-0.41 (m, 2H), 0.18-0.02 (m, 1H) ESI-MS m/z calc. 506.19876, found 507.2 (M+1)⁺; Retention time: 1.54 minutes; LC method A.

### Example 42: Preparation of Compound 38

### Step 1: tert-Butyl N-[(1R)-1-methyl-2-oxo-ethyl]carbamate

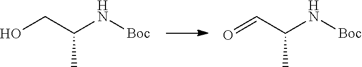

To a solution of tert-butyl N-[(1R)-2-hydroxy-1-methyl-ethyl]carbamate (200 g, 1.141 mol) in DCM (3 L) was added Dess-Martin periodinane (625 g, 1.474 mol) (fine suspension, most into solution, started exotherm, controlled with ice-bath). To the mixture was added water (28 mL, 1.554 mol) slowly added over 0.5 h (exothermic during addition up to 33° C., kept between 20 and 33° C. by cooling with cold water) giving a colorless thick suspension. The suspension was stirred at room temperature for 16 h. The solid was removed by filtration over Celite and washed 3× with 100 mL of DCM. The solvent was removed in vacuo affording an off-white slurry, which was diluted with MTBE (750 mL). The slurry was cooled with an ice-bath and filtered over Celite. The filtrate was washed 3× with sat sodium bicarbonate, brine, dried over magnesium sulfate, filtered and concentrated in vacuo. The semi-solid was re-dissolved in MTBE (300 mL) and diluted with heptane (750 mL). The solution was concentrated in vacuo until a cloud point occurred. The slurry was stirred at ambient temperature for 0.5 h. The precipitate was collected, washed with cold heptane and dried in vacuo at ambient temperature (this solid was product and was therefore kept aside). The filtrate was further concentrated in vacuo until a cloud point occurred. The solution was allowed to stand for 48 h affording a thick off-white slurry. The slurry was filtered, and the filter cake was washed with ˜50 mL of cold heptane. The filter cake was combined with the solid kept aside earlier and air-dried for 4 h. Product contained approximately 9% residual heptane by ¹H NMR. tert-Butyl N-[(1R)-1-methyl-2-oxo-ethyl]carbamate (95.6 g, 48%), ¹H NMR (500 MHz, DMSO-d₆) δ 9.43 (s, 1H), 7.35 (d, J 6.8 Hz, 1H), 3.86 (t, J 7.2 Hz, 1H), 1.40 (s, 9H), 1.13 (d, J 7.3 Hz, 3H).

### Step 2: tert-Butyl N-[(1R,2R)-2-(4-tert-butylphenyl)-2-hydroxy-1-methyl-ethyl]carbamate

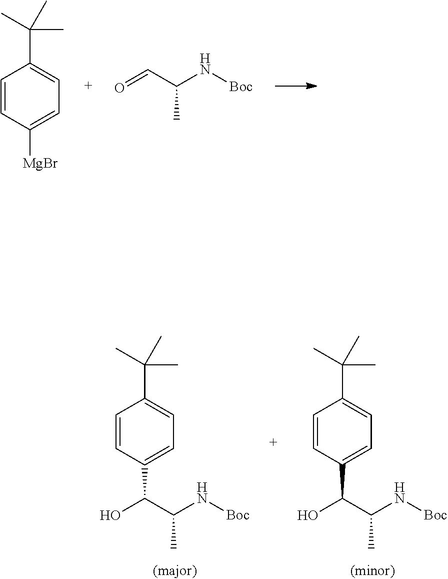

A solution of tert-butyl N-[(1R)-1-methyl-2-oxo-ethyl]carbamate (101.73 g, 587.3 mmol) in MeTHF (500 mL) was added slowly over 1 h to bromo-(4-tert-butylphenyl)magnesium (1300 mL of 1 M, 1.300 mol) (1 M in MeTHF) in a −35° C. cold bath at a rate which maintained an internal temperature between −2° C. and −15° C. After the addition was complete, it was stirred for 5 min, then the mixture was removed from the cold bath and transferred to a room temperature water bath, then stirred for 2.5 h. The mixture was cooled to 0° C., then saturated ammonium chloride (1700 mL) was added (large exotherm) at a rate which maintained an internal temperature of 5° C. Water (500 mL) was added, the organic layer was separated and washed with brine (500 mL), dried over magnesium sulfate, then concentrated under vacuum to give a light yellow oil, tert-butyl N-[(1R,2R)-2-(4-tert-butylphenyl)-2-hydroxy-1-methyl-ethyl]carbamate (266 g, >100% yield), which was used in the next step without further purification. ESI-MS m/z calc. 307.21475, found 308.1 (M+1)⁺; Retention time: 1.86 minutes; LC method A.

### Step 3: (1R,2R)-2-Amino-1-(4-tert-butylphenyl)propan-1-ol (Hydrochloride Salt)

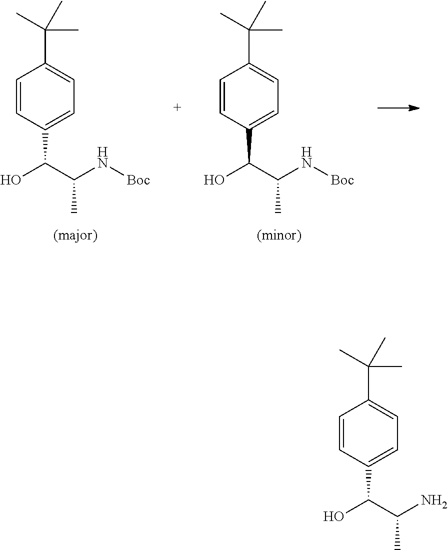 [SMILES: C[C@@H](NC(=O)OC(C)(C)C)[C@H](O)c1ccc(C(C)(C)C)cc1 ; C[C@@H](NC(=O)OC(C)(C)C)[C@@H](O)c1ccc(C(C)(C)C)cc1 ; C[C@@H](N)[C@H](O)c1ccc(C(C)(C)C)cc1]

A solution of tert-butyl N-[(1R,2R)-2-(4-tert-butylphenyl)-2-hydroxy-1-methyl-ethyl]carbamate (180.6 g, 587.5 mmol) in MeOH (250 mL) was added dropwise over 50 min to HCl in dioxane (478 mL of 4 M, 1.912 mol), maintaining a temperature between 18° C. and 23° C., then stirred at room temperature for 2 h. The mixture was concentrated under vacuum to give 267.5 g of residue. This was recrystallized from dioxane, the product was collected by filtration, then rinsed with MeTHF until all the color was removed, giving 75.4 g of product. This was further recrystallized from MeOH/dioxane, which gave (1R,2R)-2-amino-1-(4-tert-butylphenyl)propan-1-ol (hydrochloride salt) (62.65 g, 44%); ¹H NMR (500 MHz, DMSO-d₆) δ 8.10 (s, 3H), 7.39 (d, J 8.2 Hz, 2H), 7.28 (d, J 8.1 Hz, 2H), 6.12 (d, J 3.8 Hz, 1H), 4.50-4.34 (m, 1H), 3.28-3.12 (m, 1H), 1.27 (s, 9H), 0.96 (d, J 6.6 Hz, 3H). ESI-MS m/z calc. 207.16231, found 208.2 (M+1)⁺; Retention time: 1.01 minutes; LC method A.

### Step 4: (10R,11R)-10-(4-tert-Butylphenyl)-6-(2,6-dimethylphenyl)-11-methyl-9-oxa-2λ⁶-thia-3,5,12,19-tetraazatricyclo[12.3.1.14,8]nonadeca-1(18),4,6,8(19),14,16-hexaene-2,2,13-trione (Compound 38)

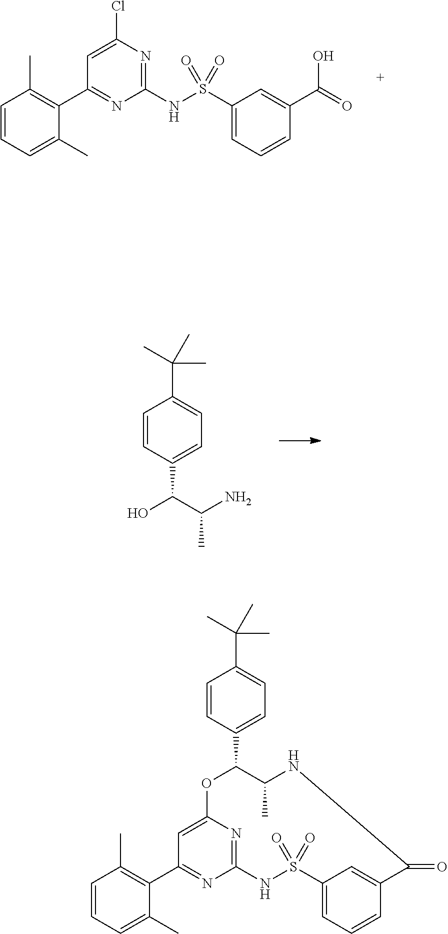 [SMILES: Cc1cccc(C)c1-c1cc(Cl)nc(NS(=O)(=O)c2cccc(C(=O)O)c2)n1 ; C[C@@H](N)[C@H](O)c1ccc(C(C)(C)C)cc1 ; Cc1cccc(C)c1-c1cc2nc(n1)NS(=O)(=O)c1cccc(c1)C(=O)N[C@H](C)[C@@H](c1ccc(C(C)(C)C)cc1)O2]

NaOtBu (59.2 g, 616.0 mmol) was added to MeTHF (750 mL) at −10° C., followed by addition of (1R,2R)-2-amino-1-(4-tert-butylphenyl)propan-1-ol (hydrochloride salt) (25 g, 102.6 mmol). After stirring for 15 min, 3-[[4-chloro-6-(2,6-dimethylphenyl)pyrimidin-2-yl]sulfamoyl]benzoic acid (42.9 g, 102.7 mmol) was added at −14° C. The temperature was then maintained at 20° C. and stirred for 5 h. The mixture was added by cannula over 35 min to a stirring solution of HATU (78.1 g, 205.4 mmol) in DMF (1,200 mL), which was immersed in a 15° C. water bath, then stirred overnight at room temperature. The solvent was evaporated under vacuum, citric acid (4,104 mL of 0.2 M, 820.8 mmol) was added, and the mixture was stirred for 2 h. The crude product was collected by filtration and rinsed with 3×1000 mL water. The product was dissolved in DCM, dried over magnesium sulfate, concentrated, then the purity was improved by normal phase silica chromatography using a gradient of 0% to 100% EtOAc/hexanes. The obtained material was recrystallized from DCM/isopropanol, then purified by reverse-phase C₁₈ chromatography using acetonitrile/water to give 24 g impure product. The 24 g was recrystallized from acetonitrile to give (10R,11R)-10-(4-tert-butylphenyl)-6-(2,6-dimethylphenyl)-11-methyl-9-oxa-2λ⁶-thia-3,5,12,19-tetraazatricyclo[12.3.1.14,8]nonadeca-1(18),4,6,8(19),14,16-hexaene-2,2,13-trione (22.57 g, 39%). ¹HNMR (500 MHz, DMSO-d₆) δ 13.02 (s, 1H), 8.69 (s, 1H), 7.93 (s, 1H), 7.68 (s, 2H), 7.55 (d, J 9.6 Hz, 1H), 7.53-7.45 (m, 4H), 7.32-7.21 (m, 1H), 7.19-7.06 (m, 2H), 6.51-6.31 (m, 2H), 3.71 (s, 1H), 2.06 (s, 6H), 1.32 (s, 9H), 0.97 (d, J=6.6 Hz, 3H). ESI-MS m/z calc. 570.2301, found 571.4 (M+1)⁺; Retention time: 1.95 minutes; LC method A.

### Example 43: Preparation of Compound 39 and Compound 40

### Step 1: tert-Butyl N-[(1R)-1-formyl-3-methyl-butyl]carbamate

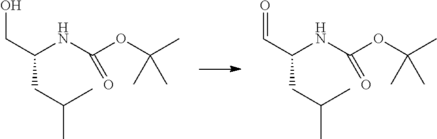

In a 500-mL round-bottomed flask, tert-butyl N-[(1R)-1-(hydroxymethyl)-3-methyl-butyl]carbamate (7.405 g, 33.05 mmol) was dissolved in DCM (150 mL), to which Dess-Martin periodinane (19.988 g, 47.13 mmol) and water (1.0 mL, 55.51 mmol) were added. The resulting slurry was stirred vigorously at room temperature under nitrogen for 33 h. Since the reaction was incomplete, more Dess-Martin periodinane (9.582 g, 22.59 mmol) and water (0.5 mL, 27.75 mmol) were added. The resulting slurry was stirred vigorously at room temperature under nitrogen for 42 h. Fine white solid was removed by vacuum filtration and the solid was rinsed with diethylether. The solid was discarded. The filtrate was evaporated in vacuo to give a white slurry, which was again filtered in vacuo and rinsed with diethylether. The solid was discarded, and the resulting filtrate was again evaporated in vacuo to give a cloudy yellow liquid, which was filtered in vacuo and rinsed with diethylether. The remaining oil was diluted with diethylether (150 mL) and subjected to aqueous workup. Then, the organic layer was washed with saturated aqueous sodium bicarbonate solution (3×150 mL). The precipitated white solid that floated above the aqueous layer was removed along with the aqueous layer at every wash. Finally, the organic layer was washed with saturated aqueous sodium chloride solution (150 mL), then dried over magnesium sulfate, filtered, and evaporated in vacuo. The residue was a cloudy, slightly yellow oil that was filtered one last time under vacuum and rinsed with hexanes (50 mL); the formed white solid was discarded. The remaining solution was evaporated in vacuo to give a slightly yellow liquid, tert-butyl N-[(1R)-1-formyl-3-methyl-butyl]carbamate (5.0454 g, 71%) ESI-MS m/z calc. 215.15215, found 160.1 (M+H-tBu+H)+; Retention time: 1.3 minutes; LC method A.

### Step 2: tert-Butyl N-[(1R)-1-[(4-tert-butylphenyl)-hydroxy-methyl]-3-methyl-butyl]carbamate

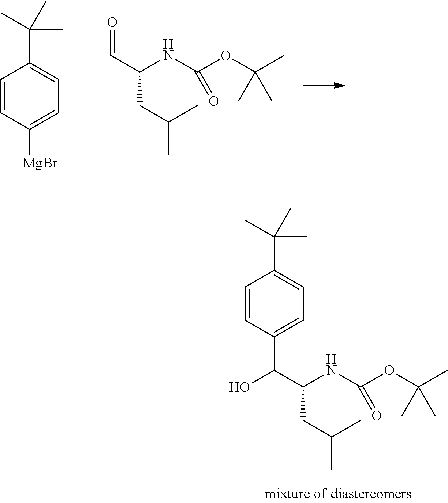

A THE solution of bromo-(4-tert-butylphenyl)magnesium (29 mL of 0.5 M, 14.50 mmol) was added to THE (12.5 mL) and the reaction mixture cooled to −8° C. (ice/brine bath), then a solution of tert-butyl N-[(1R)-1-formyl-3-methyl-butyl]carbamate (1.25 g, 5.806 mmol) in THE (5 mL) was added dropwise. The cooling bath was removed, and the reaction mixture stirred for 4 h. The reaction mixture was cooled to 0° C. and slowly quenched with saturated aqueous ammonium chloride, then poured into water and extracted with EtOAc (3×). The organics were combined, washed with water and brine, dried over sodium sulfate and evaporated to dryness. Purification by column chromatography (80 g silica; 0-40% EtOAc in hexanes) gave a white foam, tert-butyl N-[(1R)-1-[(4-tert-butylphenyl)-hydroxy-methyl]-3-methyl-butyl]carbamate (1 g, 49%); ESI-MS m/z calc. 349.2617, found 351.3 (M+1)⁺; Retention time: 0.83 minutes; LC method J with a 1 min run.

### Step 3: (2R)-2-Amino-1-(4-tert-butylphenyl)-4-methyl-pentan-1-ol

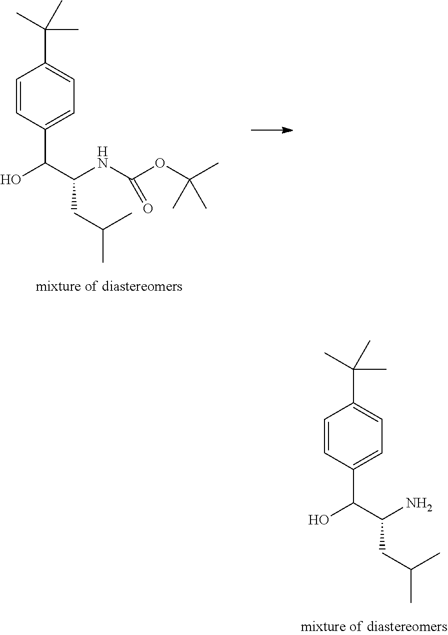 [SMILES: CC(C)C[C@@H](NC(=O)OC(C)(C)C)C(O)c1ccc(C(C)(C)C)cc1 ; CC(C)C[C@@H](N)C(O)c1ccc(C(C)(C)C)cc1]

To a solution of tert-butyl N-[(1R)-1-[(4-tert-butylphenyl)-hydroxy-methyl]-3-methyl-butyl]carbamate (1 g, 2.861 mmol) in DCM (10 mL) was added HCl in dioxane (10 mL of 4 M, 40.00 mmol) and the reaction mixture was stirred at room temperature for 2 h. The reaction mixture was evaporated to dryness and the solid was triturated with 2:1 hexanes:ether to give a white solid, (2R)-2-amino-1-(4-tert-butylphenyl)-4-methyl-pentan-1-ol (hydrochloride salt) (310 mg, 38%); ESI-MS m/z calc. 249.20926, found 250.2 (M+1)⁺; Retention time: 0.5 minutes; LC method D.

### Step 4: (11R)-10-(4-tert-Butylphenyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one, peak 1 (Compound 39), and (11R)-10-(4-tert-butylphenyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one, peak 2 (Compound 40)

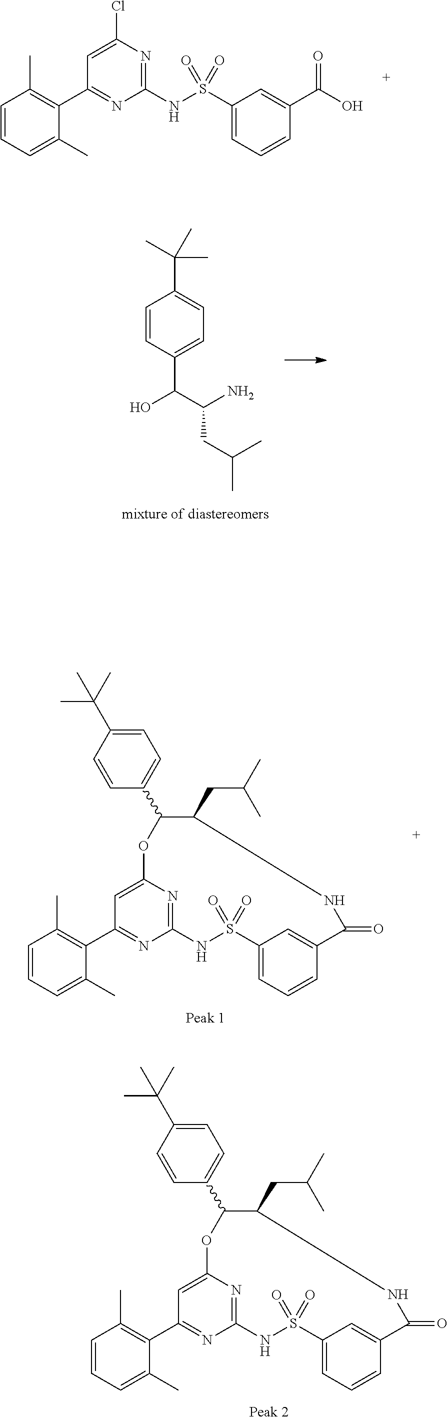 [SMILES: Cc1cccc(C)c1-c1cc(Cl)nc(NS(=O)(=O)c2cccc(C(=O)O)c2)n1 ; CC(C)C[C@@H](N)C(O)c1ccc(C(C)(C)C)cc1 ; Cc1cccc(C)c1-c1cc2nc(n1)NS(=O)(=O)c1cccc(c1)C(=O)N[C@H](CC(C)C)C(c1ccc(C(C)(C)C)cc1)O2 ; Cc1cccc(C)c1-c1cc2nc(n1)NS(=O)(=O)c1cccc(c1)C(=O)N[C@H](CC(C)C)C(c1ccc(C(C)(C)C)cc1)O2]

To a 0° C. solution of (2R)-2-amino-1-(4-tert-butylphenyl)-4-methyl-pentan-1-ol (hydrochloride salt) (110 mg, 0.3848 mmol) and 3-[[4-chloro-6-(2,6-dimethylphenyl)pyrimidin-2-yl]sulfamoyl]benzoic acid (145 mg, 0.3470 mmol) in THE (3 mL) was added NaOtBu (175 mg, 1.821 mmol) and the reaction mixture was stirred at 0° C. for 10 min. The cooling bath was removed and the reaction mixture was stirred at room temperature for 4 h, then at 40° C. for 30 min. The reaction mixture was then added onto a solution of HATU (275 mg, 0.7232 mmol) in DMF (2 mL) dropwise, and the resulting mixture was stirred at room temperature for 2 h. The reaction mixture was poured into water and extracted with EtOAc (3×), washed with water, brine, dried over sodium sulfate and evaporated to dryness. Purification by reverse-phase preparative HPLC (C₁₈) gave two products: Peak 1, earlier retention time, first to elute: (11R)-10-(4-tert-butylphenyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (9 mg, 21%) (ESI-MS m/z calc. 612.27704, found 613.4 (M+1)⁺; Retention time: 2.09 minutes; LC method A. Peak 2, later retention time, second to elute, (11R)-10-(4-tert-butylphenyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (25 mg, 15%) (ESI-MS m/z calc. 612.27704, found 613.3 (M+1)⁺; Retention time: 2.15 minutes; LC method A.

### Example 44: Preparation of Compound 41 and Compound 42

### Step 1: tert-Butyl N-[(1R,2R)-2-hydroxy-1-methyl-2-[4-(trifluoromethyl)phenyl]ethyl]carbamate, and tert-Butyl N-[(1R,2S)-2-hydroxy-1-methyl-2-[4-(trifluoromethyl)phenyl]ethyl]carbamate

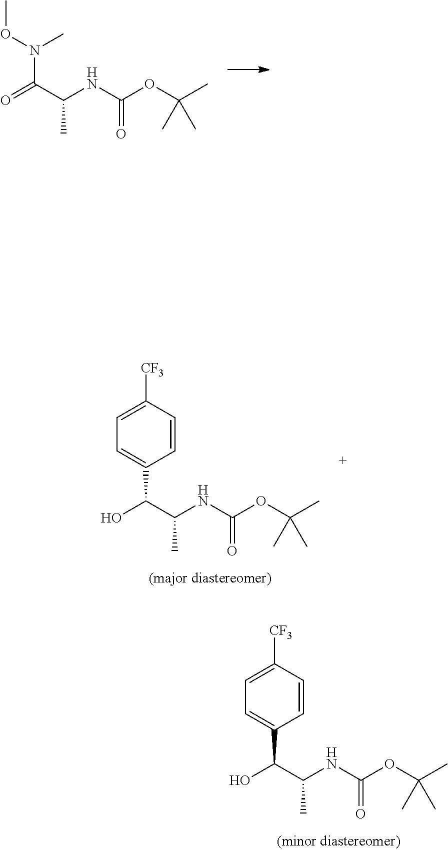 [SMILES: CON(C)C(=O)[C@@H](C)NC(=O)OC(C)(C)C ; C[C@@H](NC(=O)OC(C)(C)C)[C@H](O)c1ccc(C(F)(F)F)cc1 ; C[C@@H](NC(=O)OC(C)(C)C)[C@@H](O)c1ccc(C(F)(F)F)cc1]

Stage 1: In a 250-mL round-bottomed flask, tert-butyl N-[(1R)-2-[methoxy(methyl)amino]-1-methyl-2-oxo-ethyl]carbamate (4.01 g, 17.26 mmol) was dissolved in THE (90 mL), and this solution was cooled to 0° C. A THE solution of LAH (10.0 mL of 2.0 M, 20.00 mmol) was added dropwise, and this mixture was stirred at 0° C. for 30 min. Then, an aqueous solution of 10% citric acid (150 mL) was added dropwise, and this quenched mixture was warmed to room temperature over 1 h. The mixture was extracted with ethyl acetate (3×150 mL). The combined organic extracts was washed with water (100 mL) and saturated aqueous sodium chloride solution (100 mL), then dried over sodium sulfate, filtered, and evaporated in vacuo to give an off-white solid, tert-butyl N-[(1R)-1-methyl-2-oxo-ethyl]carbamate (3.028 g, quantitative). This crude product was taken onto the next step without purification.

Stage 2: A 100-mL round-bottomed flask containing Mg (1.8 g, 74.06 mmol) and a magnetic stir bar was dried with a heat gun under high vacuum. After the flask was allowed to cool to room temperature, it was filled with nitrogen gas. Then, diethylether (10 mL) was added, followed by a drop of 1,2-dibromoethane. This mixture was stirred vigorously for 10 min. Then, a solution of 1-bromo-4-(trifluoromethyl)benzene (10.22 g, 45.42 mmol) in diethylether (20 mL) was added portion-wise (˜2 mL volume at a time). The reaction mixture was cooled with an ice bath as necessary to avoid overheating and solvent evaporation. After the aryl bromide was completely added to the flask, the mixture was allowed to stir at room temperature for 5 min.

In a 250-mL round-bottomed flask, the product from Stage 1 (459.1 mg, 2.020 mmol) was dissolved in THE (75 mL), and this solution was cooled to 0° C. The Grignard reagent prepared above was added dropwise via syringe, and this mixture was warmed to room temperature while stirring over 15 min. It was then quenched with 1 N HCl (100 mL) and extracted with ethyl acetate (3×150 mL). The combined organic extracts were washed with water (200 mL) and saturated aqueous sodium chloride solution (200 mL), then dried over sodium sulfate, filtered, and evaporated in vacuo. The resulting black oil was purified by silica gel chromatography (120 g of silica) using a gradient eluent of 1 to 40% ethyl acetate in hexanes to give a light brown solid. This was a mixture of tert-butyl N-[(1R,2R)-2-hydroxy-1-methyl-2-[4-(trifluoromethyl)phenyl]ethyl]carbamate (major) and tert-butyl N-[(1R,2S)-2-hydroxy-1-methyl-2-[4-(trifluoromethyl)phenyl]ethyl]carbamate (minor) (combined for 2.5486 g, 33%). Major diastereomer: ¹H NMR (400 MHz, dimethylsulfoxide-d₆) δ 7.72-7.60 (m, 2H), 7.55-7.42 (m, 2H), 6.38 (d, J 8.5 Hz, 1H), 5.51 (d, J 5.0 Hz, 1H), 4.66 (t, J 4.3 Hz, 1H), 3.73 (dqd, J 8.5, 6.8, 4.3 Hz, 1H), 1.29 (s, 9H), 0.95 (d, J 6.8 Hz, 3H). Minor diastereomer: ¹H NMR (400 MHz, dimethylsulfoxide-d₆) δ 7.72-7.60 (m, 2H), 7.55-7.42 (m, 2H), 6.71 (d, J 8.9 Hz, 1H), 5.57 (d, J 4.8 Hz, 1H), 4.49 (t, J 5.6 Hz, 1H), 3.60-3.49 (m, 1H), 1.24 (s, 9H), 1.00 (d, J=6.8 Hz, 3H). ESI-MS m/z calc. 319.13953, found 320.3 (M+1)⁺; Retention time: 1.6 minutes; LC method A.

### Step 2: (1R,2R)-2-Amino-1-[4-(trifluoromethyl)phenyl]propan-1-ol, and (1R,2S)-2-Amino-1-[4-(trifluoromethyl)phenyl]propan-1-ol

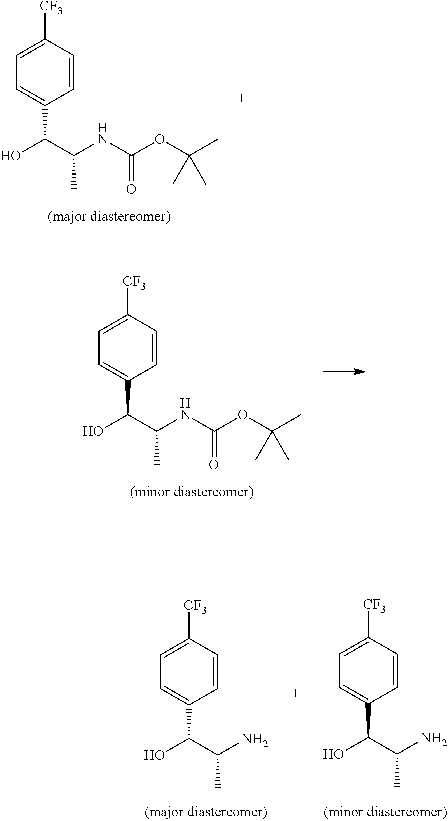 [SMILES: C[C@@H](NC(=O)OC(C)(C)C)[C@H](O)c1ccc(C(F)(F)F)cc1 ; C[C@@H](NC(=O)OC(C)(C)C)[C@@H](O)c1ccc(C(F)(F)F)cc1 ; C[C@@H](N)[C@H](O)c1ccc(C(F)(F)F)cc1 ; C[C@@H](N)[C@@H](O)c1ccc(C(F)(F)F)cc1]

In a 100-mL round-bottomed flask, tert-butyl N-[(1R,2R)-2-hydroxy-1-methyl-2-[4-(trifluoromethyl)phenyl]ethyl]carbamate (2.5486 g, 5.747 mmol) was dissolved in dioxane (3.0 mL), to which a dioxane solution of HCl (9.0 mL of 4.0 M, 36.00 mmol) was added. This mixture was stirred at room temperature for 15 h, after which it was evaporated to dryness in vacuo; THF (20 mL) was added and re-evaporated in vacuo to give 2.10 g of an orange foam. This was a mixture of (1R,2R)-2-amino-1-[4-(trifluoromethyl)phenyl]propan-1-ol (hydrochloride salt) (major) and (1R,2S)-2-amino-1-[4-(trifluoromethyl)phenyl]propan-1-ol (minor) (combined for 2.109 g, 101%). ESI-MS m/z calc. 219.0871, found 220.1 (M+1)⁺; Retention time: 0.72 minutes; LC method A.

### Step 3: (10R,11R)-6-(2,6-Dimethylphenyl)-11-methyl-2,2-dioxo-10-[4-(trifluoromethyl)phenyl]-9-oxa-2λ⁶-thia-3,5,12,19-tetrazatricyclo[12.3.1.14,8]nonadeca-1(18),4(19),5,7,14,16-hexaen-13-one, major diastereomer (Compound 41), and (10S,11R)-6-(2,6-dimethylphenyl)-11-methyl-2,2-dioxo-10-[4-(trifluoromethyl)phenyl]-9-oxa-2λ⁶-thia-3,5,12,19-tetrazatricyclo[12.3.1.14,8]nonadeca-1(18),4(19),5,7,14,16-hexaen-13-one, minor diastereomer (Compound 42)

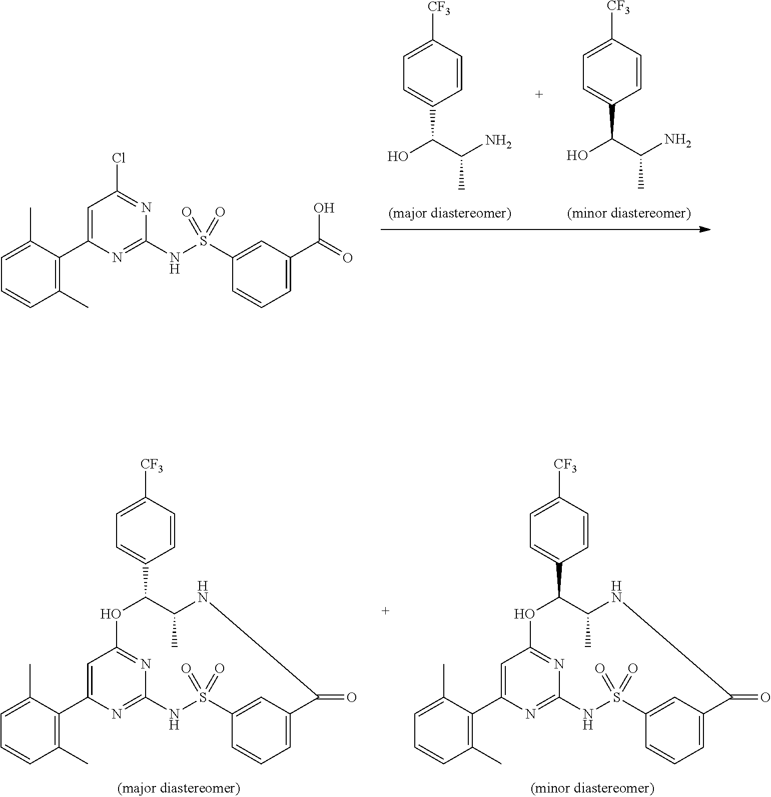 [SMILES: Cc1cccc(C)c1-c1cc(Cl)nc(NS(=O)(=O)c2cccc(C(=O)O)c2)n1 ; C[C@@H](N)[C@H](O)c1ccc(C(F)(F)F)cc1 ; C[C@@H](N)[C@@H](O)c1ccc(C(F)(F)F)cc1 ; Cc1cccc(C)c1-c1cc2nc(n1)NS(=O)(=O)c1cccc(c1)C(=O)N[C@H](C)[C@@H](c1ccc(C(F)(F)F)cc1)O2 ; Cc1cccc(C)c1-c1cc2nc(n1)NS(=O)(=O)c1cccc(c1)C(=O)N[C@H](C)[C@H](c1ccc(C(F)(F)F)cc1)O2]

In a 20-mL vial, (1R,2R)-2-amino-1-[4-(trifluoromethyl)phenyl]propan-1-ol (hydrochloride salt) (422.0 mg, 1.155 mmol) was dissolved in THE (4.0 mL), to which NaOtBu (1.005 g, 10.46 mmol) was added. This mixture was stirred at room temperature for 10 min, after which it was cooled to 0° C. Then, 3-[[4-chloro-6-(2,6-dimethylphenyl)pyrimidin-2-yl]sulfamoyl]benzoic acid (501.2 mg, 1.174 mmol) was added, and this mixture was stirred at 0° C. for 1 h, then at room temperature for 1 h.

In a 250-mL round-bottomed flask, a solution of HATU (1.009 g, 2.654 mmol) in DMF (8.0 mL) was prepared. The above-prepared reaction mixture was added dropwise onto this HATU solution, and the resulting mixture was stirred at room temperature for 15 min. Then, a second portion of HATU (0.6658 g, 1.751 mmol) was added, and stirred for 5 min. This mixture was then quenched with 1 N HCl solution (40 mL), and diluted with ethyl acetate (150 mL). The layers were separated, and the organic layer was washed with 1 N HCl solution (50 mL), water (50 mL) and saturated aqueous sodium chloride solution (50 mL), then dried over sodium sulfate, filtered, and evaporated in vacuo. The resulting slurry was purified by silica gel chromatography (24 g of silica column) using a gradient eluent of 0 to 40% ethyl acetate in hexanes, then re-purified by reverse-phase preparative chromatography using a C₁₈ column and a gradient eluent of 1 to 70% acetonitrile in water containing 5 mM hydrochloric acid to give 2 batches of product: Major product, (10R,11R)-6-(2,6-dimethylphenyl)-11-methyl-2,2-dioxo-10-[4-(trifluoromethyl)phenyl]-9-oxa-2λ⁶-thia-3,5,12,19-tetrazatricyclo[12.3.1.14,8]nonadeca-1(18),4(19),5,7,14,16-hexaen-13-one (68.2 mg, 10%); ¹H NMR (400 MHz, dimethylsulfoxide-d₆) δ 13.68-11.37 (bs, 1H), 8.76 (s, 1H), 7.95 (s, 1H), 7.89-7.80 (m, 4H), 7.70 (s, 1H), 7.63 (d, J 9.6 Hz, 1H), 7.27 (t, J 7.6 Hz, 1H), 7.13 (d, J 7.6 Hz, 2H), 6.56 (d, J 4.3 Hz, 1H), 6.47 (s, 1H), 3.85-3.72 (m, 1H), 2.07 (s, 6H), 0.92 (d, J 6.7 Hz, 3H). ESI-MS m/z calc. 582.15485, found 583.3 (M+1)⁺; Retention time: 1.73 minutes; LC method A; and minor product, (10S,11R)-6-(2,6-dimethylphenyl)-11-methyl-2,2-dioxo-10-[4-(trifluoromethyl)phenyl]-9-oxa-2λ⁶-thia-3,5,12,19-tetrazatricyclo[12.3.1.14,8]nonadeca-1(18),4(19),5,7,14,16-hexaen-13-one (25.1 mg, 4%); ¹H NMR (400 MHz, dimethylsulfoxide-d₆) δ 13.71-11.22 (bs, 1H), 8.57-8.31 (bs, 1H), 8.27-8.01 (bs, 1H), 7.97-7.76 (m, 5H), 7.74-7.51 (m, 2H), 7.46-6.68 (bs, 1H), 7.25 (t, J 7.6 Hz, 1H), 7.11 (d, J 7.6 Hz, 2H), 6.63-6.18 (bs, 1H), 3.92-3.60 (m, 1H), 1.99 (s, 6H), 0.81-0.21 (m, 3H). ESI-MS m/z calc. 582.15485, found 583.3 (M+1)⁺; Retention time: 1.69 minutes; LC method A.

### Example 45: Preparation of Compound 43

### Step 1: tert-Butyl N-[(1S)-2-(4-tert-butylphenyl)-1-methyl-2-oxo-ethyl]carbamate

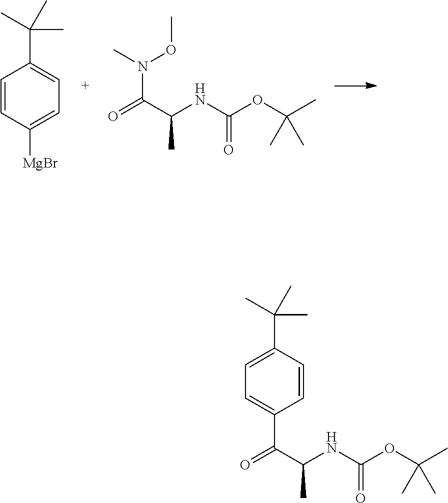

In a 250-mL round-bottomed flask, tert-butyl N-[(1S)-2-[methoxy(methyl)amino]-1-methyl-2-oxo-ethyl]carbamate (1.1377 g, 4.653 mmol) was mixed with THE (50 mL), and cooled to 0° C. Then, an diethylether solution of bromo-(4-tert-butylphenyl)magnesium (25.0 mL of 0.5 M, 12.50 mmol) was added in one portion. This mixture was allowed to warm to room temperature over 20 h. It was then quenched with 1 N HCl (100 mL), and extracted with ethyl acetate (3×100 mL). The combined organic extracts was washed with water (200 mL) and saturated aqueous sodium chloride solution (200 mL), then dried over sodium sulfate, filtered, and evaporated in vacuo. The resulting yellow oil was purified by silica gel chromatography (120 g of silica) using a gradient eluent of 0 to 30% ethyl acetate in hexanes to give a viscous yellow oil, tert-butyl N-[(1S)-2-(4-tert-butylphenyl)-1-methyl-2-oxo-ethyl]carbamate (1.367 g, 96%); ESI-MS m/z calc. 305.1991, found 306.3 (M+1)⁺; Retention time: 1.94 minutes; LC method A.

### Step 2: tert-Butyl N-[(1S,2R)-2-(4-tert-butylphenyl)-2-hydroxy-1-methyl-ethyl]carbamate, major diastereomer, and tert-Butyl N-[(1S,2S)-2-(4-tert-butylphenyl)-2-hydroxy-1-methyl-ethyl]carbamate, minor diastereomer

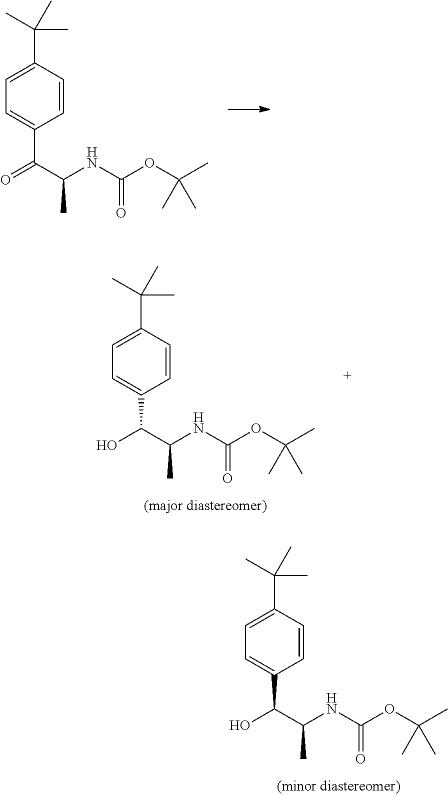 [SMILES: C[C@H](NC(=O)OC(C)(C)C)C(=O)c1ccc(C(C)(C)C)cc1 ; C[C@H](NC(=O)OC(C)(C)C)[C@H](O)c1ccc(C(C)(C)C)cc1 ; C[C@H](NC(=O)OC(C)(C)C)[C@@H](O)c1ccc(C(C)(C)C)cc1]

tert-Butyl N-[(1S)-2-(4-tert-butylphenyl)-1-methyl-2-oxo-ethyl]carbamate (1.367 g, 4.476 mmol) in MeOH (100 mL) was cooled to 0° C. and treated with sodium borohydride (350 mg, 9.251 mmol). This solution was stirred at 0° C. for 15 min, after which a second portion of sodium borohydride (350 mg, 9.251 mmol) was added. The resulting mixture was stirred at 0° C. for 2 h, after which a third portion of sodium borohydride (950 mg, 25.11 mmol) was added. The reaction mixture was stirred at 0° C. for 30 min, after which it was quenched with 1 N HCl solution (200 mL). The mixture was extracted with ethyl acetate (2×250 mL). The combined organic extracts was washed with water (300 mL) and saturated aqueous sodium chloride solution (300 mL), then dried over sodium sulfate, filtered, and evaporated in vacuo. The resulting brown oil was purified by silica gel chromatography (80 g of silica) using a gradient eluent of 0 to 7% methanol in dichloromethane, then re-purified by an SFC purification method using a ChiralPak IG column (250×21.2 mm, 5 m particle size) at 40° C., with a mobile phase of 14% MeOH+86% CO₂, a flow rate of 70 mL/min, an injection volume of 500 μL, and a pressure of 100 bar. This gave two products: Major diastereomer, tert-butyl N-[(1S,2R)-2-(4-tert-butylphenyl)-2-hydroxy-1-methyl-ethyl]carbamate (465.8 mg, 34%); ¹H NMR (400 MHz, dimethylsulfoxide-d₆) δ 7.30 (d, J 8.4 Hz, 2H), 7.22 (d, J 8.3 Hz, 2H), 6.57 (d, J 8.8 Hz, 1H), 5.20 (d, J 4.5 Hz, 1H), 4.43 (t, J 5.2 Hz, 1H), 3.67-3.50 (m, 1H), 1.28 (s, 9H), 1.25 (s, 9H), 0.95 (d, J 6.7 Hz, 3H). ESI-MS m/z calc. 307.21475, found 308.3 (M+1)⁺; Retention time: 7.45 minutes; LC method A with a 1-99% over 13.5 min gradient of phase B; and minor diastereomer, tert-butyl N-[(1S,2S)-2-(4-tert-butylphenyl)-2-hydroxy-1-methyl-ethyl]carbamate (138.7 mg, 10%); ¹H NMR (400 MHz, dimethylsulfoxide-d₆) δ 7.36-7.28 (m, 2H), 7.24-7.14 (m, 2H), 6.31 (d, J 8.2 Hz, 1H), 5.18 (d, J 4.8 Hz, 1H), 4.47 (t, J 5.1 Hz, 1H), 3.71-3.58 (m, 1H), 1.33 (s, 9H), 1.26 (s, 9H), 0.88 (d, J 6.8 Hz, 3H). ESI-MS m/z calc. 307.21475, found 308.3 (M+1)⁺; Retention time: 7.45 minutes; LC method A with a 1-99% over 13.5 min gradient of phase B.

### Step 3: (1R,2S)-2-Amino-1-(4-tert-butylphenyl)propan-1-ol

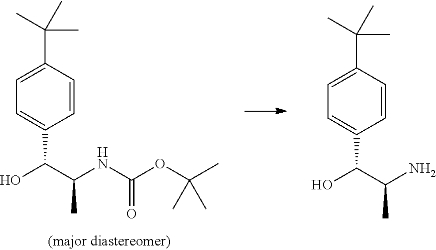 [SMILES: C[C@H](NC(=O)OC(C)(C)C)[C@H](O)c1ccc(C(C)(C)C)cc1 ; C[C@H](N)[C@H](O)c1ccc(C(C)(C)C)cc1]

In a 20-mL vial, tert-butyl N-[(1S,2R)-2-(4-tert-butylphenyl)-2-hydroxy-1-methyl-ethyl]carbamate (295.9 mg, 0.9625 mmol) was dissolved in dioxane (3.0 mL), to which a dioxane solution of HCl (3.0 mL of 4.0 M, 12.00 mmol) was added. This mixture was stirred at room temperature for 6 h, after which it was evaporated in vacuo to give ˜300 mg of an off-white solid (>100% yield). Purification by reverse-phase preparative chromatography using a Cis column and a gradient eluent of 1 to 50% acetonitrile in water containing 5 mM hydrochloric acid gave (1R,2S)-2-amino-1-(4-tert-butylphenyl)propan-1-ol (hydrochloride salt) (181.9 mg, 78%)¹H NMR (400 MHz, dimethylsulfoxide-d₆) δ 8.19 (s, 3H), 7.42-7.35 (m, 2H), 7.32-7.25 (m, 2H), 5.97 (d, J 4.1 Hz, 1H), 4.94 (t, J 3.5 Hz, 1H), 3.34 (qd, J 6.6, 2.7 Hz, 1H), 1.27 (s, 9H), 0.95 (d, J 6.7 Hz, 3H). ESI-MS m/z calc. 207.16231, found 208.1 (M+1)⁺; Retention time: 0.91 minutes; LC method A.

### Step 4: (10R,11S)-10-(4-tert-Butylphenyl)-6-(2,6-dimethylphenyl)-11-methyl-2,2-dioxo-9-oxa-2)⁶-thia-3,5,12,19-tetrazatricyclo[12.3.1.14,8]nonadeca-1(18),4(19),5,7,14,16-hexaen-13-one (Compound 43)

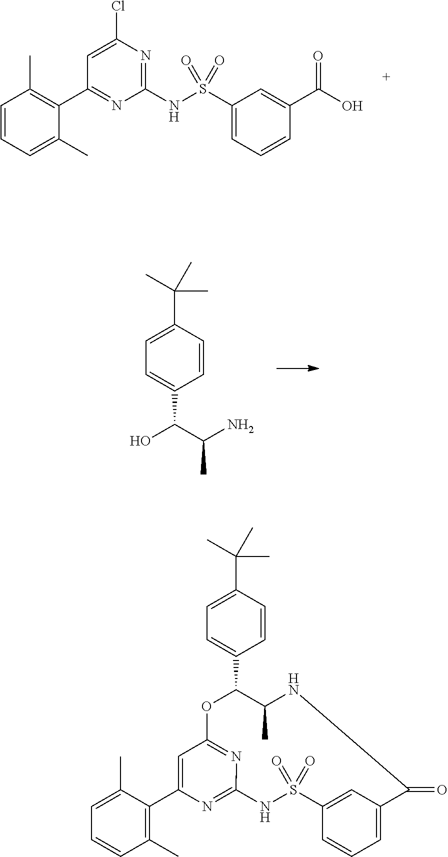 [SMILES: Cc1cccc(C)c1-c1cc(Cl)nc(NS(=O)(=O)c2cccc(C(=O)O)c2)n1 ; C[C@H](N)[C@H](O)c1ccc(C(C)(C)C)cc1 ; Cc1cccc(C)c1-c1cc2nc(n1)NS(=O)(=O)c1cccc(c1)C(=O)N[C@@H](C)[C@@H](c1ccc(C(C)(C)C)cc1)O2]

In a 3-mL vial, (1R,2S)-2-amino-1-(4-tert-butylphenyl)propan-1-ol (hydrochloride salt) (55.0 mg, 0.2256 mmol), NaOtBu (100.2 mg, 1.043 mmol) and THE (1.0 mL) were added. This mixture was stirred at room temperature for 10 min, after which it was cooled to 0° C. Then, 3-[[4-chloro-6-(2,6-dimethylphenyl)pyrimidin-2-yl]sulfamoyl]benzoic acid (67.0 mg, 0.1603 mmol) was added, and the resulting mixture was stirred at 0° C. for 1 h, then at room temperature for 1 h. In a 20-mL vial, a solution of HATU (137.2 mg, 0.3608 mmol) in DMF (2.0 mL) was prepared. The reaction mixture prepared above was added dropwise into this HATU solution over 1 min. The resulting mixture was stirred at room temperature for 5 min, after which it was quenched with 1 N HCl solution (5 mL) and extracted with ethyl acetate (3×4 mL). The combined organic extracts were washed with water (10 mL) and saturated aqueous sodium chloride solution (10 mL), then dried over sodium sulfate, filtered, and evaporated in vacuo. Purification by reverse-phase preparative chromatography using a C₁₈ column and a gradient eluent of 1 to 99% acetonitrile in water containing 5 mM hydrochloric acid gave (10R,11S)-10-(4-tert-butylphenyl)-6-(2,6-dimethylphenyl)-11-methyl-2,2-dioxo-9-oxa-2λ⁶-thia-3,5,12,19-tetrazatricyclo[12.3.1.14,8]nonadeca-1(18),4(19),5,7,14,16-hexaen-13-one (12.0 mg, 13%); ¹H NMR (400 MHz, dimethylsulfoxide-d₆) δ 13.40-10.99 (s, 1H), 8.40 (s, 1H), 8.16 (s, 1H), 7.88 (s, 1H), 7.74-7.53 (m, 2H), 7.55-7.40 (m, 4H), 7.31-7.18 (m, 1H), 7.11 (d, J 7.6 Hz, 2H), 7.15-6.81 (bs, 1H), 6.28 (s, 1H), 3.97-3.67 (m, 1H), 2.19-1.81 (bs, 6H), 1.31 (s, 9H), 0.85-0.24 (bs, 3H) ESI-MS m/z calc. 570.2301, found 571.3 (M+1)⁺; Retention time: 1.84 minutes; LC method A.

### Example 46: Preparation of Compound 44 and Compound 45

### Step 1: tert-Butyl N-[(1R)-1-(methoxymethyl)-2-[methoxy(methyl)amino]-2-oxo-ethyl]carbamate

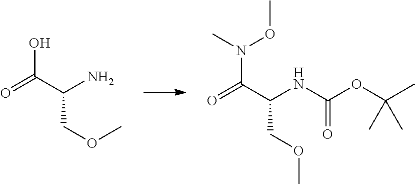

To a solution of (2R)-2-amino-3-methoxy-propanoic acid (2.01 g, 16.87 mmol) in THE (15 mL) and water (15 mL) at 0° C. was added sodium bicarbonate (4.29 g, 51.07 mmol) followed by Boc anhydride (5.7 g, 26.12 mmol) and the stirred mixture was slowly allowed to warm to room temperature over 16 h. The reaction mixture was quenched with saturated aqueous KHSO₄, bringing the pH to ˜5, then extracted with EtOAc (4×). The organic layers were combined, washed with water, brine, dried over sodium sulfate and evaporated to dryness. The residue was taken up in DMF (20 mL) and to this solution was added 1-hydroxybenzotriazole (2.5 g, 18.50 mmol), DIPEA (8.8 mL, 50.52 mmol) and EDCI-HCl (3.56 g, 18.57 mmol), stirred for 2 min, then N-methoxymethanamine (hydrochloride salt) (2.5 g, 25.63 mmol) and more DIPEA (4.4 mL, 25.26 mmol) were added. The reaction mixture was then stirred at room temperature for 16 h. The reaction mixture was poured into 0.1 N HCl and extracted with EtOAc (3×). The organics were combined, washed with 0.1 N HCl (2×), saturated aqueous sodium bicarbonate (2×), water, and brine, then dried over sodium sulfate and evaporated to dryness. Purification by column chromatography (0-70% EtOAc in hexanes) gave a clear oil, tert-butyl N-[(1R)-1-(methoxymethyl)-2-[methoxy(methyl)amino]-2-oxo-ethyl]carbamate (3.47 g, 78%); ESI-MS m/z calc. 262.15286, found 263.1 (M+1)⁺; Retention time: 0.42 minutes; LC method A.

### Step 2: tert-Butyl N-[(1R)-1-formyl-2-methoxy-ethyl]carbamate, and tert-Butyl N-[(1R,2R)-2-(4-tert-butylphenyl)-2-hydroxy-1-(methoxymethyl)ethyl]carbamate

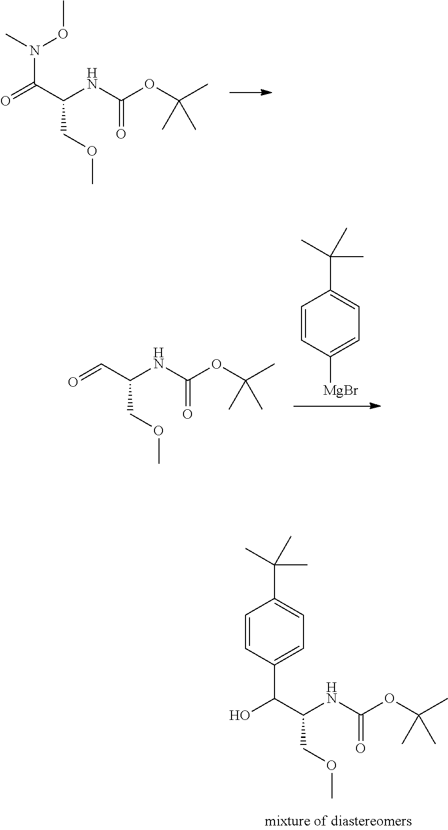

To a 0° C. solution of tert-butyl N-[(1R)-1-(methoxymethyl)-2-[methoxy(methyl)amino]-2-oxo-ethyl]carbamate (330 mg, 1.258 mmol) in THE (3 mL) was added LAH (2 M in THF) (700 μL of 2 M, 1.400 mmol) dropwise and the reaction mixture was stirred at 0° C. for 1 h. The reaction mixture was quenched with ice, the pH was brought to ˜5 with 0.1 N HCl and then extracted with EtOAc (3×). The organics were combined, washed with water, brine, dried over sodium sulfate, filtered through a short plug of silica gel, and evaporated to dryness. The residue was taken up in THE (3 mL), cooled to 0° C. and bromo-(4-tert-butylphenyl)magnesium (5.6 mL of 0.5 M, 2.800 mmol) was added dropwise. The cooling bath was removed and the reaction mixture was stirred for 2 h. The reaction mixture was poured into water and the pH brought to ˜5 with 0.1 N HCl and then extracted with EtOAc (3×). The organics were combined, washed with water, brine, dried over sodium sulfate, and evaporated to dryness. Purification by column chromatography gave tert-butyl N-[(1R,2R)-2-(4-tert-butylphenyl)-2-hydroxy-1-(methoxymethyl)ethyl]carbamate (100 mg, 24%) as a ˜1:2 mix of diastereomers. ESI-MS m/z calc. 337.2253, found 338.4 (M+1)⁺; Retention time: 0.69 minutes; LC method Q using a 1 min gradient.

### Step 3: 3-[[4-[(1R,2R)-2-Amino-1-(4-tert-butylphenyl)-3-methoxy-propoxy]-6-(2,6-dimethylphenyl)pyrimidin-2-yl]sulfamoyl]benzoic Acid

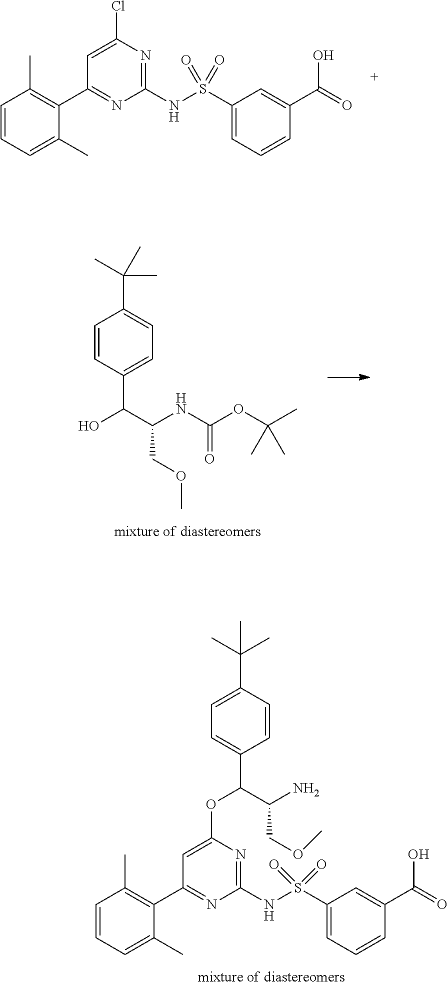 [SMILES: Cc1cccc(C)c1-c1cc(Cl)nc(NS(=O)(=O)c2cccc(C(=O)O)c2)n1 ; COC[C@@H](NC(=O)OC(C)(C)C)C(O)c1ccc(C(C)(C)C)cc1 ; COC[C@@H](N)C(Oc1cc(-c2c(C)cccc2C)nc(NS(=O)(=O)c2cccc(C(=O)O)c2)n1)c1ccc(C(C)(C)C)cc1]

To a solution of tert-butyl N-[(1R,2R)-2-(4-tert-butylphenyl)-2-hydroxy-1-(methoxymethyl)ethyl]carbamate (100 mg, 0.2963 mmol) in DCM (3 mL) was added HCl in dioxane (1.5 mL of 4 M, 6.000 mmol) and the reaction mixture was stirred at room temperature for 1 h, then evaporated to dryness. The residue was taken up in THF (2 mL), 3-[[4-chloro-6-(2,6-dimethylphenyl)pyrimidin-2-yl]sulfamoyl]benzoic acid (121 mg, 0.2835 mmol) was added, the solution cooled to 0° C. and sodium t-butoxide (159 mg, 1.654 mmol) was added. The cooling bath was removed the reaction mixture was stirred at room temperature for 2 h. Then, NaH (12 mg of 60% w/w, 0.3000 mmol) was added and the reaction mixture was stirred at 50° C. for 1 h. The reaction was cooled and evaporated to dryness. The residue was taken up in 1:1 MeOH: DMSO with a few drops of water and purified by HPLC (1-99% ACN in water (HCl modifier)) to give 3-[[4-[(1R,2R)-2-amino-1-(4-tert-butylphenyl)-3-methoxy-propoxy]-6-(2,6-dimethylphenyl)pyrimidin-2-yl]sulfamoyl]benzoic acid (hydrochloride salt) (50 mg, 26%) as a white solid. ESI-MS m/z calc. 618.2512, found 619.5 (M+1)⁺; Retention time: 0.57 minutes; LC method D.

### Step 4: (11R)-10-(4-tert-Butylphenyl)-6-(2,6-dimethylphenyl)-11-(methoxymethyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 44), and (11R)-10-(4-tert-butylphenyl)-6-(2,6-dimethylphenyl)-11-(methoxymethyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 45)

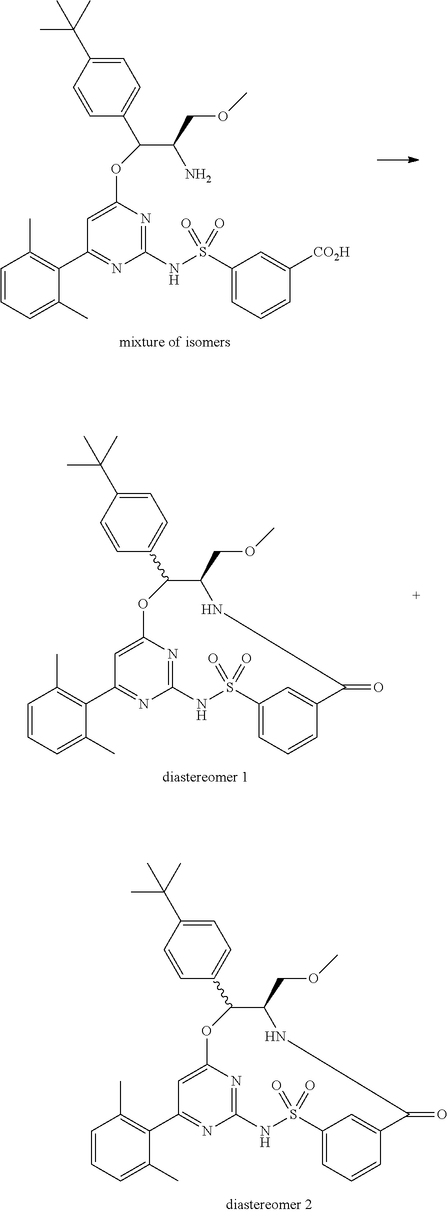 [SMILES: COC[C@@H](N)C(Oc1cc(-c2c(C)cccc2C)nc(NS(=O)(=O)c2cccc(C(=O)O)c2)n1)c1ccc(C(C)(C)C)cc1 ; COC[C@H]1NC(=O)c2cccc(c2)S(=O)(=O)Nc2nc(cc(-c3c(C)cccc3C)n2)OC1c1ccc(C(C)(C)C)cc1 ; COC[C@H]1NC(=O)c2cccc(c2)S(=O)(=O)Nc2nc(cc(-c3c(C)cccc3C)n2)OC1c1ccc(C(C)(C)C)cc1]

To a solution of 3-[[4-[(1R,2R)-2-amino-1-(4-tert-butylphenyl)-3-methoxy-propoxy]-6-(2,6-dimethylphenyl)pyrimidin-2-yl]sulfamoyl]benzoic acid (50 mg, 0.08081 mmol) in DMF (0.5 mL) was added HATU (42 mg, 0.1105 mmol) followed by DIPEA (71 μL, 0.4076 mmol), and the reaction mixture stirred at room temperature for 30 min. The reaction mixture was diluted with 1: 1 DMSO: MeOH with a few drops of water, filtered and purified by reverse-phase preparative HPLC (C₁₈) to give two products: White solid, first eluting Peak 1, diastereomer 1, (11R)-10-(4-tert-butylphenyl)-6-(2,6-dimethylphenyl)-11-(methoxymethyl)-9-oxa-2λ⁶-thia-3,5,12,19-tetraazatricyclo[12.3.1.14,8]nonadeca-1(17),4(19),5,7,14(18),15-hexaene-2,2,13-trione (1.7 mg, 8%) (ESI-MS m/z calc. 600.24066, found 601.4 (M+1)⁺; Retention time: 1.83 minutes); LC method A; white solid, second eluting eak 2, diastereomer 2, (11R)-10-(4-tert-butylphenyl)-6-(2,6-dimethylphenyl)-11-(methoxymethyl)-9-oxa-2λ⁶-thia-3,5,12,19-tetraazatricyclo[12.3.1.14,8]nonadeca-1(17),4(19),5,7,14(18),15-hexaene-2,2,13-trione (3.2 mg, 10%) (ESI-MS m/z calc. 600.24066, found 601.5 (M+1)⁺; Retention time: 1.88 minutes; LC method A.

### Example 47: Preparation of Compound 46

### Step 1: tert-Butyl N-[(1R)-1-(hydroxymethyl)-4-methyl-pentyl]carbamate

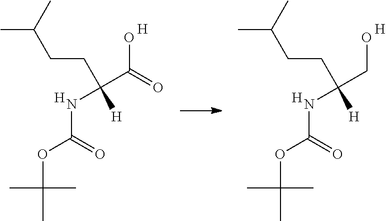

To (2R)-2-(tert-butoxycarbonylamino)-5-methyl-hexanoic acid (100 mg, 0.4076 mmol) in a vial cooled in an ice bath was added borane-THF (1.3 mL of 1 M, 1.300 mmol) in THF. The reaction mixture was then removed from the ice bath and stirred at room temperature for 2 hours. The reaction mixture was quenched by slow addition to 1 M aqueous citric acid, then extracted 3× with ethyl acetate. The combined organics were washed with brine, dried over sodium sulfate and concentrated. The resulting tert-butyl N-[(1R)-1-(hydroxymethyl)-4-methyl-pentyl]carbamate (51 mg, 54%) ESI-MS m/z calc. 231.18344, found 232.2 (M+1)⁺; Retention time: 0.59 minutes (LC method D) was used in the next step without further concentration.

### Step 2: (11R)-6-(2,6-Dimethylphenyl)-11-(3-methylbutyl)-9-oxa-2⁶-thia-3,5,12,19-tetraazatricyclo[12.3.1.14,8]nonadeca-1(17),4,6,8(19),14(18),15-hexaene-2,2,13-trione (Compound 46)

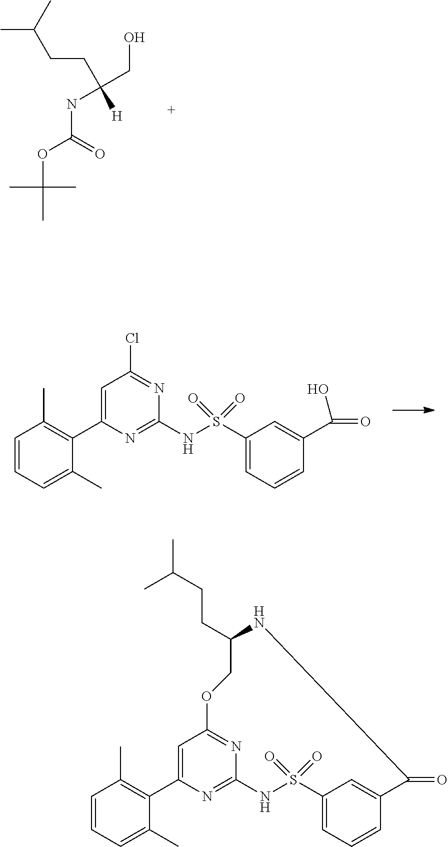 [SMILES: CC(C)CC[C@H](CO)NC(=O)OC(C)(C)C ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CCC(C)C)CO2]

Stage 1: tert-Butyl N-[(1R)-1-(hydroxymethyl)-4-methyl-pentyl]carbamate (51 mg, 0.2205 mmol) (with boc-deprotected impurity present) and 3-[[4-chloro-6-(2,6-dimethylphenyl)pyrimidin-2-yl]sulfamoyl]benzoic acid (75 mg, 0.1795 mmol) were combined with sodium tert-butoxide (110 mg, 1.145 mmol) in THE (0.5 mL) and stirred for 16 hours at room temperature. The reaction mixture was acidified with 0.3 mL acetic acid, then diluted with methanol, filtered, and purified by reverse phase HPLC (1-70% ACN in water with HCl modifier) to give after drying, 3-[[4-[(2R)-2-(tert-butoxycarbonylamino)-5-methyl-hexoxy]-6-(2,6-dimethylphenyl)pyrimidin-2-yl]sulfamoyl]benzoic acid (6 mg, 5%).The product was dissolved in dichloromethane (0.5 mL) and HCl (0.5 mL of 4 M, 2.000 mmol) (in dioxane) and stirred at room temperature for 1 hour. The reaction mixture was then concentrated to a solid residue, 3-[[4-[(2R)-2-amino-5-methyl-hexoxy]-6-(2,6-dimethylphenyl)pyrimidin-2-yl]sulfamoyl]benzoic acid (hydrochloride salt) (5 mg, 5%) which was used in the next step without further purification. ESI-MS m/z calc. 512.20935, found 513.4 (M+1)⁺; Retention time: 0.46 minutes (LC method D).

Stage 2: The product was combined with HATU (6 mg, 0.01578 mmol) in DMF (1 mL), and DIPEA (15 μL, 0.08612 mmol) was added. After stirring at room temperature for 1 hour, the reaction mixture was filtered and purified by reverse phase HPLC (1-70% ACN in water, HCl modifier, 15 min run) to give after drying, (11R)-6-(2,6-dimethylphenyl)-11-(3-methylbutyl)-9-oxa-2λ⁶-thia-3,5,12,19-tetraazatricyclo[12.3.1.14,8]nonadeca-1(17),4,6,8(19),14(18),15-hexaene-2,2,13-trione (2.5 mg, 3%) ESI-MS m/z calc. 494.19876, found 495.4 (M+1)⁺; Retention time: 1.62 minutes (LC method A).

### Example 48: Preparation of Compound 47

### Step 1: Methyl 2-(tert-butoxycarbonylamino)-5,5-dimethyl-hex-2-enoate

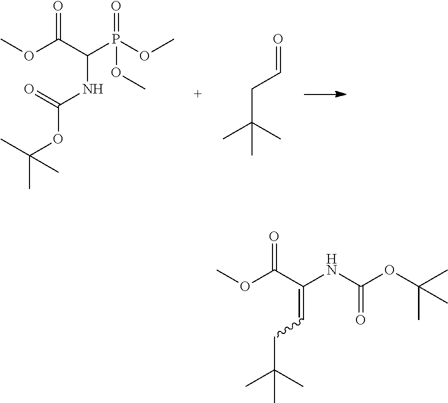

To a stirred solution of methyl 2-(tert-butoxycarbonylamino)-2-dimethoxyphosphoryl-acetate (2.86 g, 9.6218 mmol) and DBU (1.4252 g, 1.4 mL, 9.3617 mmol) in DCM (20 mL) was added 3,3-dimethylbutyraldehyde (997.50 mg, 1.25 mL, 8.7358 mmol). The reaction mixture was stirred at room temperature for 16 h. Aqueous HCl (1 N) (25 mL) was added and the phases were separated. The aqueous layer was washed with DCM (2×20 mL). The combined organic layers were dried over magnesium sulfate, filtered and concentrated under reduced pressure. The crude residue was purified by chromatography on a 40 g silica gel cartridge using a gradient of 0-30% EtOAc in heptanes to afford methyl 2-(tert-butoxycarbonylamino)-5,5-dimethyl-hex-2-enoate (2.305 g, 97%) as a clear oil that crystallized to a white solid. ESI-MS m/z calc. 271.1784, found 216.4 (M−55)⁺; Retention time: 1.91 minutes LC method X. ¹H NMR (400 MHz, CDCl₃) δ 6.67 (t, J 7.6 Hz, 1H), 5.86 (br. s., 1H), 3.79 (s, 3H), 2.12 (d, J 7.6 Hz, 2H), 1.47 (s, 9H), 0.96 (s, 9H).

### Step 2: Methyl (2R)-2-(tert-butoxycarbonylamino)-5,5-dimethyl-hexanoate

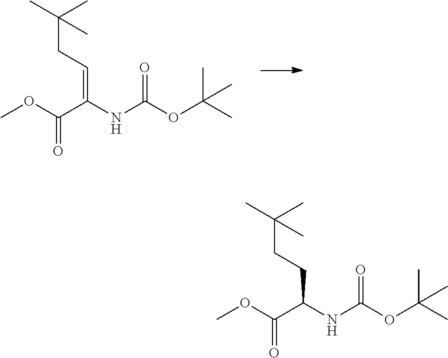 [SMILES: COC(=O)/C(=C\CC(C)(C)C)NC(=O)OC(C)(C)C ; COC(=O)[C@@H](CCC(C)(C)C)NC(=O)OC(C)(C)C]

To a solution of methyl (E)-2-(tert-butoxycarbonylamino)-4,5,5-trimethyl-hex-2-enoate (2 g, 7.0082 mmol) in ethanol (27 mL) and 1,4-dioxane (9 mL) was bubbled nitrogen for 5 min. Then, 1,2-bis[(2R,5R)-2,5-diethylphospholano]benzene(1,5-cyclooctadiene)rhodium(I) trifluoromethanesulfonate (51 mg, 0.0706 mmol) was added and the mixture was put in an ultrasound bath for 5 min. under nitrogen. The reaction mixture was hydrogenated under 50 psi (3.5 bar) of hydrogen pressure and at room temperature for 16 h. Silica gel was added to the reaction mixture and it was evaporated to dryness. The product was purified by chromatography on a 40 g silica gel cartridge using a gradient of 0-30% EtOAc in heptanes to afford methyl (2R)-2-(tert-butoxycarbonylamino)-5,5-dimethyl-hexanoate (1.91 g, 100%). ESI-MS m/z calc. 273.194, found 218.4 (M−55)⁺; Retention time: 1.96 minutes, LC method X. ¹H NMR (400 MHz, CDCl₃) δ 5.08-4.88 (m, 1H), 4.36-4.21 (m, 1H), 3.75 (s, 3H), 1.85-1.74 (m, 1H), 1.67-1.59 (m, 1H), 1.45 (s, 9H), 1.26-1.16 (m, 2H), 0.87 (s, 9H).

### Step 3: tert-Butyl N-[(1R)-1-(hydroxymethyl)-4,4-dimethyl-pentyl]carbamate

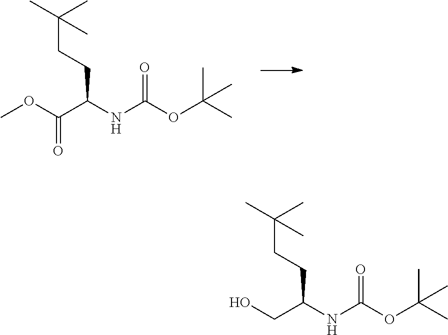 [SMILES: COC(=O)[C@@H](CCC(C)(C)C)NC(=O)OC(C)(C)C ; CC(C)(C)CC[C@H](CO)NC(=O)OC(C)(C)C]

To a solution of methyl (2R)-2-(tert-butoxycarbonylamino)-5,5-dimethyl-hexanoate (1.9 g, 6.9503 mmol) in THE (20 mL) was added LiBH4 (2 M solution in THF) (8.8 mL of 2 M, 17.600 mmol). The reaction mixture was stirred at room temperature for 2.5 h. The reaction mixture was then poured slowly over a saturated aqueous solution of ammonium chloride (50 mL) at 0° C. (strong evolution of gas, but no exotherm). The product was extracted with EtOAc (3×50 mL). The combined organic layers were washed with brine (50 mL), dried over magnesium sulfate, filtered and concentrated under reduced pressure to afford crude product tert-butyl N-[(1R)-1-(hydroxymethyl)-4,4-dimethyl-pentyl]carbamate (1.725 g, 101%) as a clear oil. ¹H NMR. ESI-MS m/z calc. 245.1991, found 190.2 (M−55)⁺; Retention time: 1.81 minutes. ¹H NMR (400 MHz, CDCl₃) δ 4.65-4.51 (m, 1H), 3.74-3.65 (m, 1H), 3.62-3.51 (m, 2H), 2.35 (br. s., 1H), 1.46 (s, 9H), 1.42-1.17 (m, 4H), 0.89 (s, 9H). LC method X.

### Step 4: (2R)-2-Amino-5,5-dimethyl-hexan-1-ol

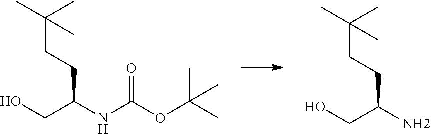

To a solution of tert-butyl N-[(1R)-1-(hydroxymethyl)-4,4-dimethyl-pentyl]carbamate (1.72 g, 7.0102 mmol) in 1,4-dioxane (9 mL) was added hydrogen chloride (4 N in 1,4-dioxane) (9 mL of 4 M, 36.000 mmol). The reaction mixture was stirred at room temperature for 16 h. The mixture was evaporated to give (2R)-2-amino-5,5-dimethyl-hexan-1-ol (hydrochloride salt) (1.19 g, 93%) as a white solid. ¹H NMR (400 MHz, DMSO-d₆) δ 7.81 (br. s., 3H), 5.26 (t, J 4.9 Hz, 1H), 3.58 (dt, J 11.5, 4.0 Hz, 1H), 3.47-3.37 (m, 1H), 2.98 (br. s., 1H), 1.53-1.41 (m, 2H), 1.26-1.14 (m, 2H), 0.87 (s, 9H). ESI-MS m/z calc. 145.14667, found 146.4 (M+1)⁺; Retention time: 1.05 minutes; LC method X.

### Step 5: tert-Butyl N-[1-(hydroxymethyl)-4,4-dimethyl-pentyl]carbamate

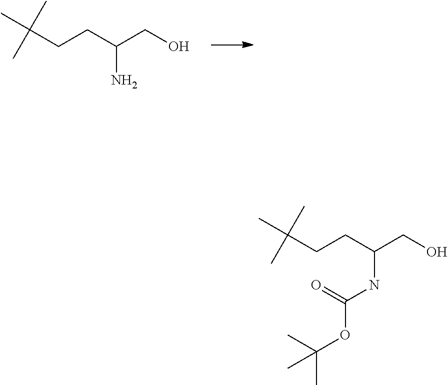

2-Amino-5,5-dimethyl-hexan-1-ol (hydrochloride salt) (254 mg, 1.398 mmol), Boc anhydride (320 mg, 1.466 mmol), and cesium carbonate (1.2 g, 3.683 mmol) were combined in THE (5 mL) and stirred for 3 h. The reaction was partitioned between ethyl acetate and a 0.3M HCl solution. The organics were separated, washed with brine, dried over sodium sulfate and evaporated. The resulting oil was further dried to give a waxy solid, used in the next step without further purification (some Boc anhydride contamination) tert-butyl N-[1-(hydroxymethyl)-4,4-dimethyl-pentyl]carbamate (320 mg, 93%). ESI-MS m/z calc. 245.1991, found 246.3 (M+1)⁺; Retention time: 0.63 minutes ¹H NMR (400 MHz, DMSO) δ 6.42 (d, J 8.0 Hz, 1H), 4.52 (t, J 5.3 Hz, 1H), 3.34-3.31 (m, 1H), 3.24-3.18 (m, 1H), 1.37 (s, 9H), 1.26-1.14 (m, 2H), 1.14-1.05 (m, 1H), 0.84 (s, 9+1H). LC method D.

### Step 6: (11R)-11-(3,3-Dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, peak 1 (Compound 47), and (11S)-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, peak 2 (Compound 48)

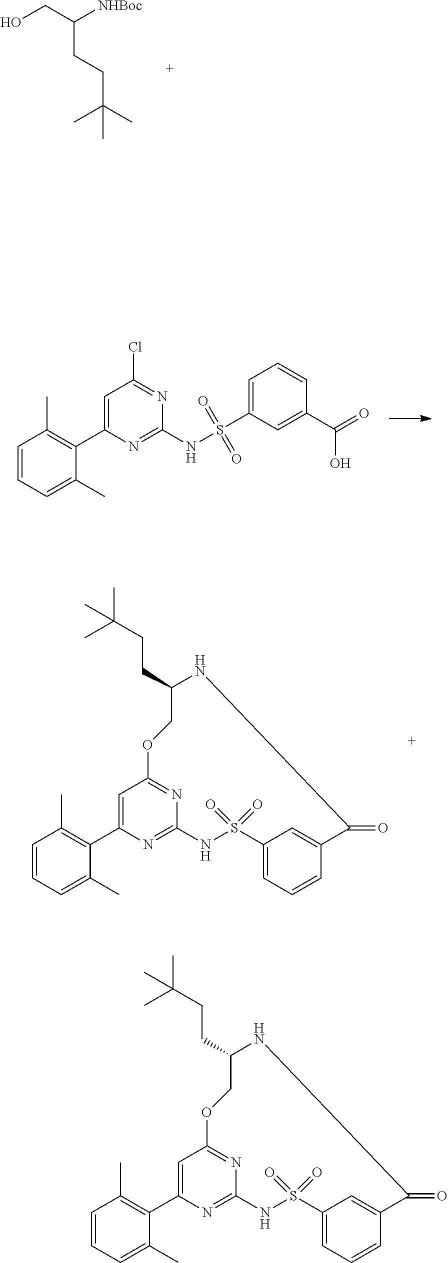 [SMILES: CC(C)(C)CCC(CO)NC(=O)OC(C)(C)C ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CCC(C)(C)C)CO2 ; Cc1cccc(C)c1-c1cc2nc(n1)NS(=O)(=O)c1cccc(c1)C(=O)N[C@@H](CCC(C)(C)C)CO2]

Stage 1: tert-Butyl N-[1-(hydroxymethyl)-4,4-dimethyl-pentyl]carbamate (230 mg, 0.9374 mmol) and 3-[[4-chloro-6-(2,6-dimethylphenyl)pyrimidin-2-yl]sulfamoyl]benzoic Acid (200 mg, 0.4786 mmol) were combined with sodium tert-butoxide (230 mg, 2.393 mmol) in THE (1.25 mL) and stirred for 2 hours at room temperature. The reaction mixture was acidified with 0.5 mL acetic acid, then added to 1 M citric acid, and extracted 3× with ethyl acetate. The combined organics were washed with water, then brine, and dried over sodium sulfate. The resulting crude material was purified by flash chromatography on silica gel (0-10% methanol in DCM) to give as a solid residue, 3-[[4-[2-(tert-butoxycarbonylamino)-5,5-dimethyl-hexoxy]-6-(2,6-dimethylphenyl)pyrimidin-2-yl]sulfamoyl]benzoic acid (100 mg, 33%) ESI-MS m/z calc. 626.2774, found 627.5 (M+1)⁺; Retention time: 0.77 minutes (LC method D).

Stage 2: The product was dissolved in dichloromethane (3 mL) and HCl (1.5 mL of 4 M, 6.000 mmol) (in dioxane) and stirred at room temperature for 1 hour. The reaction mixture was then concentrated to a solid residue, hexanes were added, and the reaction mixture was again concentrated to give 3-[[4-(2-amino-5,5-dimethyl-hexoxy)-6-(2,6-dimethylphenyl)pyrimidin-2-yl]sulfamoyl]benzoic acid (hydrochloride salt) (90 mg, 33%) ESI-MS m/z calc. 526.225, found 527.4 (M+1)⁺; Retention time: 0.5 minutes (LC method D), which was used in the next step without further purification.

Stage 3: The product was combined with HATU (75 mg, 0.1972 mmol) in DMF (15 mL), and DIPEA (135 μL, 0.7751 mmol) was added. After stirring at room temperature for 1 hour, the reaction mixture was filtered and purified by reverse phase HPLC (1-70% ACN in water, HCl modifier, 15 min run) to give after drying, 11-(3,3-dimethylbutyl)-6-(2,6-dimethylphenyl)-2,2-dioxo-9-oxa-2σ⁶-thia-3,5,12,19-tetrazatricyclo[12.3.1.14,8]nonadeca-1(18),4(19),5,7,14,16-hexaen-13-one (80 mg, 33%). This material was then subjected to chiral SFC (Phenomenex LUX-2 (250×21.2 mm, 5 m column, mobile phase: 42% MeCN/MeOH (90:10:20 mM NH₃) and 54% CO₂, flow: 70 mL/min, concentration 8 mg/mL in MeCN/MeOH/DMSO (81:9:10), injection volue: 750 μL, 100 bar, 210 nm) to give two enantiomers: Peak 1: (11R)-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (7 mg, 3%) ESI-MS m/z calc. 508.21442, found 509.4 (M+1)⁺; Retention time: 1.75 minutes (LC method A); and peak 2: (11S)-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (7 mg, 3%) ESI-MS m/z calc. 508.21442, found 509.4 (M+1)⁺; Retention time: 1.75 minutes (LC method A).

### Example 49: Preparation of Compound 49

### Step 1: Spiro[3.3]heptan-2-ylmethanol

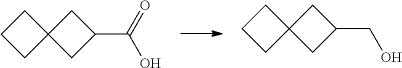 [SMILES: O=C(O)C1CC2(CCC2)C1 ; OCC1CC2(CCC2)C1]

To a solution of spiro[3.3]heptane-2-carboxylic acid (9.5 g, 67.770 mmol) in tetrahydrofuran (190 mL) cooled in an ice bath, was added dropwise Lithium aluminum hydride (in THF) (82 mL of 1 M, 82.000 mmol) over 15 minutes, maintaining an internal temperature <5° C. After the addition was complete, the reaction was stirred at 0-5° C. for 1 hour and at room temperature for 2 hours. The resulting mixture was cooled in an ice bath and water (10 mL) was added dropwise. Aqueous sodium hydroxide (15% w/w, 10 mL) was then added followed by additional water (25 mL). The reaction mixture was stirred for 15 minutes at room temperature and then it was filtered and rinsed with THF. The filtrate was concentrated in vacuo and the residue was diluted in EtOAc (100 mL) and washed with brine (20 mL). The organic phase was concentrated in vacuo to afford spiro[3.3]heptan-2-ylmethanol (8.4 g, 93%) as a light yellow oil. H NMR (400 MHz, CDCl₃) δ 3.55 (d, J 6.8 Hz, 2H), 2.39-2.28 (m, 1H), 2.11-2.03 (m, 2H), 2.03-1.96 (m, 2H), 1.93-1.86 (m, 2H), 1.83-1.76 (m, 2H), 1.74-1.66 (m, 2H), 1.48-1.37 (m, 1H).

### Step 2: Spiro[3.3]heptane-2-carbaldehyde

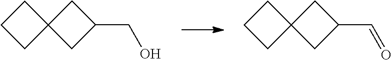 [SMILES: OCC1CC2(CCC2)C1 ; O=CC1CC2(CCC2)C1]

To a solution of spiro[3.3]heptan-2-ylmethanol (7.9 g, 59.471 mmol) in dichloromethane (160 mL) was added sodium bicarbonate (29 g, 345.21 mmol) and Dess-Martin periodinane (31 g, 73.089 mmol). The reaction mixture was stirred at room temperature 3 h. A 5% aqueous solution of sodium bicarbonate (200 mL) was added (strong evolution of gas) followed by a 10% w/w aqueous solution of Na₂S₂O₃ (200 mL). The mixture was vigorously stirred at room temperature for 3 h (until organic phase was clear). The phases were separated and the aqueous layer was extracted with DCM (2×250 mL). The combined organic layers were washed with a 10% w/w aqueous solution of Na₂S₂O₃ (200 mL), dried over magnesium sulfate, filtered and concentrated under reduced pressure to afford crude spiro[3.3]heptane-2-carbaldehyde (8.1 g, 99%) as a clear oil. ¹H NMR (400 MHz, CDCl₃) δ 9.70 (d, J 2.2 Hz, 1H), 3.07-2.97 (m, 1H), 2.27-2.13 (m, 4H), 2.07-2.01 (m, 2H), 1.94-1.88 (m, 2H), 1.85-1.77 (m, 2H).

### Step 3: methyl (Z)-2-(tert-butoxycarbonylamino)-3-spiro[3.3]heptan-2-yl-prop-2-enoate and methyl (E)-2-(tert-butoxycarbonylamino)-3-spiro[3.3]heptan-2-yl-prop-2-enoate

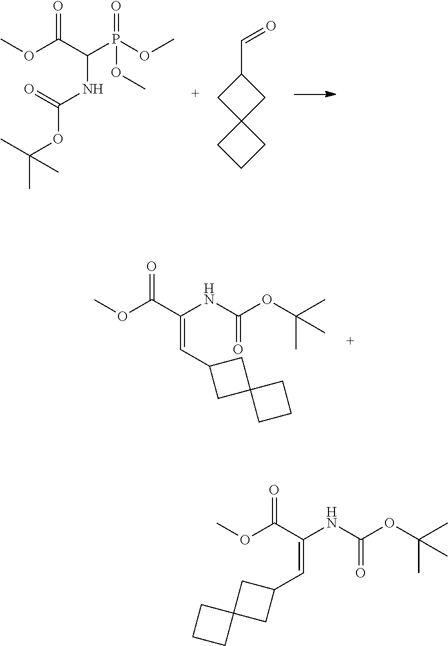

To a stirred solution of methyl 2-(tert-butoxycarbonylamino)-2-dimethoxyphosphoryl-acetate (1.3 g, 4.3735 mmol) and DBU (712.60 mg, 0.7 mL, 4.6809 mmol) in dichloromethane (10 mL) was added spiro[3.3]heptane-2-carbaldehyde (500 mg, 4.0264 mmol). The reaction mixture was stirred at room temperature for 16 h. Aqueous HCl (1 N) (10 mL) was added and the phases were separated. The aqueous layer was washed with DCM (2×20 mL). The combined organic layers were dried over magnesium sulfate, filtered and concentrated under reduced pressure. The crude residue was purified by chromatography on a silica gel cartridge (25 g+40 g) using a gradient of 0 to 30% EtOAc in heptanes to afford methyl (Z)-2-(tert-butoxycarbonylamino)-3-spiro[3.3]heptan-2-yl-prop-2-enoate (1.07 g, 90%) as a clear oil. ¹H NMR (400 MHz, CDCl₃) δ 6.58 (d, J 8.3 Hz, 1H), 5.85 (br. s., 1H), 3.80-3.73 (m, 3H), 3.15-3.04 (m, 1H), 2.33-2.23 (m, 2H), 2.06-2.01 (m, 2H), 1.94-1.75 (m, 6H), 1.51-1.42 (m, 9H). ESI-MS m/z calc. 295.1784, found 240.2 (M−55)⁺; Retention time: 1.98 minutes and methyl (E)-2-(tert-butoxycarbonylamino)-3-spiro[3.3]heptan-2-yl-prop-2-enoate (82 mg, 6%) as a yellow oil. ¹H NMR (400 MHz, CDCl₃) δ 6.80-6.78 (m, 1H), 6.53 (br. s., 1H), 3.81 (s, 3H), 3.69-3.59 (m, 1H), 2.34-2.25 (m, 2H), 2.05 (t, J 7.1 Hz, 2H), 1.92-1.76 (m, 6H), 1.47 (m, 9H). ESI-MS m/z calc. 295.1784, found 240.2 (M−55)+; Retention time: 2.05 minutes. LC method X.

### Step 4: Methyl (2R)-2-(tert-butoxycarbonylamino)-3spiro[3.3]heptan-2-yl-propanoate

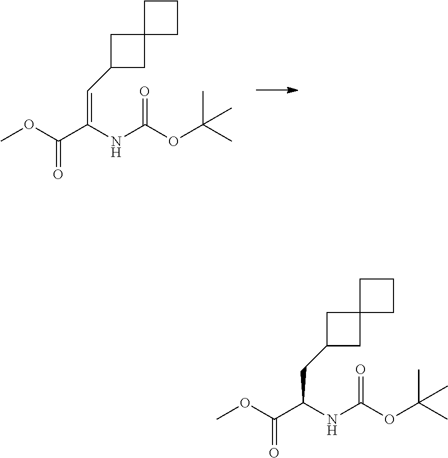 [SMILES: COC(=O)/C(=C/C1CC2(CCC2)C1)NC(=O)OC(C)(C)C ; COC(=O)[C@@H](CC1CC2(CCC2)C1)NC(=O)OC(C)(C)C]

Methyl (Z)-2-(tert-butoxycarbonylamino)-3-spiro[3.3]heptan-2-yl-prop-2-enoate (12.9 g, 42.363 mmol) was dissolved in ethanol (185 mL) and dioxane (60 mL). Nitrogen was passed through for about 10 min using a cannula. The solution was placed into an ultrasound bath (about 5 min), and 1,2-bis[(2R,5R)-2,5-diethylphospholano]benzene(1,5-cyclooctadiene)rhodium(I) trifluoromethanesulfonate (500 mg, 0.6781 mmol) was added. The mixture was hydrogenated under 3.5 bar hydrogen pressure and at room temperature for 24 hours. The reaction mixture was filtered through silica gel and the eluate was concentrated. The crude was directly purified by silica-gel column chromatography on a 100 g and 120 g column, eluting from 0 to 30% of ethyl acetate in heptanes to afford methyl (2R)-2-(tert-butoxycarbonylamino)-3-spiro[3.3]heptan-2-yl-propanoate (12.5 g, 99%) as a clear oil. ¹H NMR (400 MHz, CDCl₃) δ 4.93 (d, J 7.6 Hz, 1H), 4.26-4.15 (m, 1H), 3.72 (s, 3H), 2.23-2.05 (m, 3H), 1.98 (t, J 6.8 Hz, 2H), 1.88-1.65 (m, 6H), 1.64-1.54 (m, 2H), 1.44 (s, 9H). ESI-MS m/z calc. 297.194, found 198.2 (M−99)+; Retention time: 2.03 minutes, LC method X.

### Step 5: tert-Butyl N-[(1R)-1-(hydroxymethyl)-2-spiro[3.3]heptan-2-yl-ethyl]carbamate

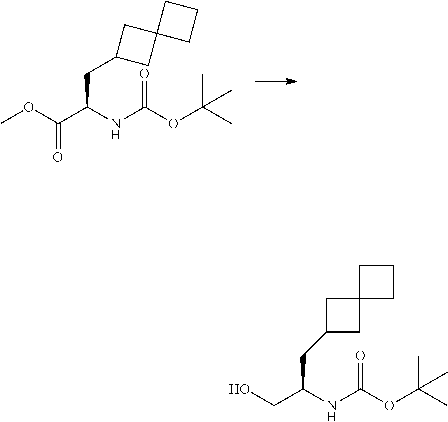 [SMILES: COC(=O)[C@@H](CC1CC2(CCC2)C1)NC(=O)OC(C)(C)C ; CC(C)(C)OC(=O)N[C@@H](CO)CC1CC2(CCC2)C1]

To a solution of methyl (2R)-2-(tert-butoxycarbonylamino)-3-spiro[3.3]heptan-2-yl-propanoate (12.5 g, 42.032 mmol) in tetrahydrofuran (125 mL) was added LiBH4 (in THF) (55 mL of 2 M, 110.00 mmol) (no exotherm observed). The reaction mixture was stirred at room temperature for 3 hours. The reaction mixture was then poured slowly over a saturated aqueous solution of ammonium chloride (150 mL) at 0° C. (strong evolution of gas, but no exotherm). The product was extracted with EtOAc (3×150 mL). The combined organic layers were washed with brine (200 mL), dried over anhydrous sodium sulfate, filtered and concentrated under reduced pressure. The crude was purified through flash column chromatography (100 g+120 g) using a gradient of 0 to 50% EtOAc in heptanes to afford tert-butyl N-[(1R)-1-(hydroxymethyl)-2-spiro[3.3]heptan-2-yl-ethyl]carbamate (11 g, 97%) as a clear oil. ¹H NMR (400 MHz, CDCl₃) δ 4.58 (br. s., 1H), 3.66-3.43 (m, 3H), 2.22-2.06 (m, 3H), 2.06-2.03 (m, 1H), 1.99 (t, J 6.8 Hz, 2H), 1.88-1.72 (m, 4H), 1.65-1.48 (m, 4H), 1.45 (s, 9H). ESI-MS m/z calc. 269.1991, found 214.2 (M−55)+; Retention time: 1.87 minutes, LC method X.

### Step 6: (2R)-2-Amino-3-spiro[3.3]heptan-2-yl-propan-1-ol

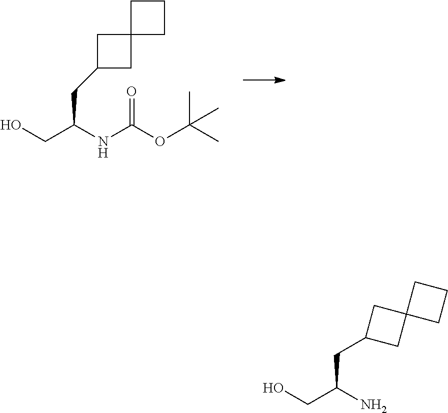 [SMILES: CC(C)(C)OC(=O)N[C@@H](CO)CC1CC2(CCC2)C1 ; N[C@@H](CO)CC1CC2(CCC2)C1]

To a solution of tert-butyl N-[(1R)-1-(hydroxymethyl)-2-spiro[3.3]heptan-2-yl-ethyl]carbamate (11 g, 40.835 mmol) in 1,4-dioxane (110 mL) was added hydrogen chloride (4 N in 1,4-dioxane) (110 mL of 4 M, 440.00 mmol). The reaction mixture was stirred at room temperature for 16 hours. The mixture was evaporated to give (2R)-2-amino-3-spiro[3.3]heptan-2-yl-propan-1-ol (hydrochloride salt) (7.8 g, 88%)(2R)-2-amino-3-spiro[3.3]heptan-2-yl-propan-1-ol (hydrochloride salt) (7.8 g, 88%) as a white solid. ¹H NMR (400 MHz, DMSO-d₆) δ 8.04 (br. s., 3H), 5.26 (br. s., 1H), 3.58-3.48 (m, 1H), 3.42-3.34 (m, 1H), 2.87 (br. s., 1H), 2.25-2.14 (m, 1H), 2.14-2.03 (m, 2H), 1.95 (t, J 7.2 Hz, 2H), 1.87-1.79 (m, 2H), 1.78-1.69 (m, 2H), 1.63-1.49 (m, 4H). ESI-MS m/z calc. 169.1467, found 170.2 (M+1)⁺; Retention time: 1.91 minutes, LC method Y.

### Step 7: 3-[[4-[(2R)-2-Amino-3-spiro[3.3]heptan-2-yl-propoxy]-6-(2,6-dimethylphenyl)pyrimidin-2-yl]sulfamoyl]benzoic Acid

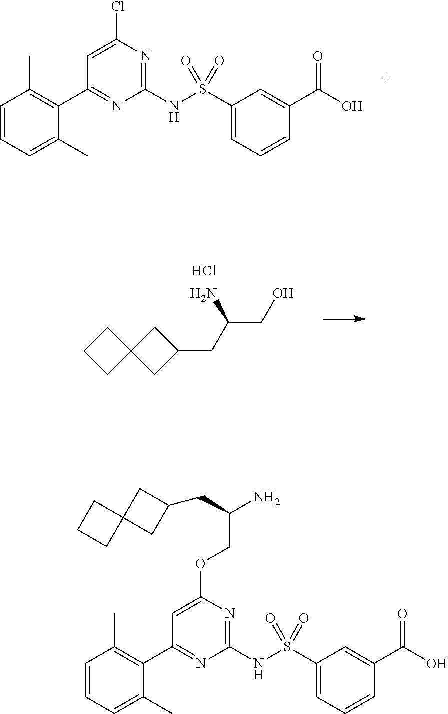 [SMILES: Cc1cccc(C)c1-c1cc(Cl)nc(NS(=O)(=O)c2cccc(C(=O)O)c2)n1 ; Cl ; N[C@@H](CO)CC1CC2(CCC2)C1 ; Cc1cccc(C)c1-c1cc(OC[C@H](N)CC2CC3(CCC3)C2)nc(NS(=O)(=O)c2cccc(C(=O)O)c2)n1]

3-[[4-Chloro-6-(2,6-dimethylphenyl)pyrimidin-2-yl]sulfamoyl]benzoic acid (1 g, 2.393 mmol) and (2R)-2-amino-3-spiro[3.3]heptan-2-yl-propan-1-ol (hydrochloride salt) (590 mg, 2.868 mmol) were combined in THE (5 mL) and stirred at room temperature for 5 minutes in a screwcap vial. Sodium tert-butoxide (1.35 g, 14.05 mmol) was then added in one portion. The reaction became warm and it was stirred for an additional 45 minutes without external heating. The reaction mixture was then partitioned between 40 mL 1M HCl and 40 mL ethyl acetate. The layers were separated, and the aqueous was extracted an additional 3× with ethyl acetate. The combined organics were washed with brine, dried over sodium sulfate, and concentrated to give as a white solid, 3-[[4-[(2R)-2-amino-3-spiro[3.3]heptan-2-yl-propoxy]-6-(2,6-dimethylphenyl)pyrimidin-2-yl]sulfamoyl]benzoic acid (hydrochloride salt) (1.455 g, 98%)/ESI-MS m/z calc. 550.225, found 551.5 (M+1)⁺; Retention time: 0.52 minutes, LC method D.

### Step 8: (11R)-6-(2,6-Dimethylphenyl)-2,2-dioxo-11-(spiro[3.3]heptan-2-ylmethyl)-9-oxa-2λ⁶-thia-3,5,12,19-tetrazatricyclo[12.3.1.14,8]nonadeca-1(18),4(19),5,7,14,16-hexaen-13-one (Compound 49)

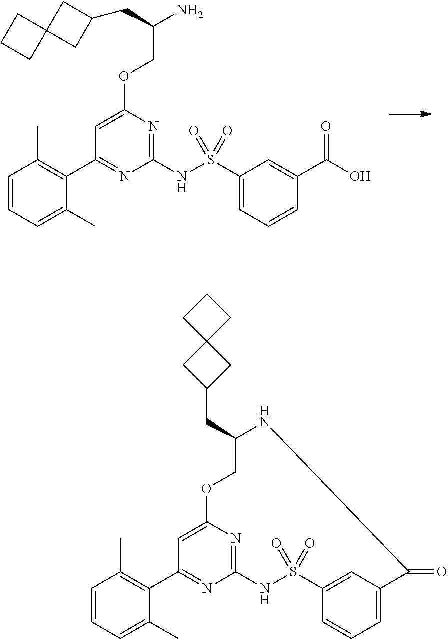 [SMILES: Cc1cccc(C)c1-c1cc(OC[C@H](N)CC2CC3(CCC3)C2)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CC1CC3(CCC3)C1)CO2]

3-[[4-[(2R)-2-Amino-3-spiro[3.3]heptan-2-yl-propoxy]-6-(2,6-dimethylphenyl)pyrimidin-2-yl]sulfamoyl]benzoic acid (hydrochloride salt) (20 mg, 0.03236 mmol) was combined with N-methylmorpholine (approximately 19.64 mg, 21.35 μL, 0.1942 mmol) in DMF (1 mL) and cooled to 0° C. CDMT (approximately 7.386 mg, 0.04207 mmol) was added and the reaction was warmed to room temperature after 30 minutes and allowed to stir for an additional hour. And the reaction mixture was then filtered and purified by reverse phase HPLC (1-99% ACN in water, HCl modifier, 15 min run) to give the corresponding (11R)-6-(2,6-dimethylphenyl)-2,2-dioxo-11-(spiro[3.3]heptan-2-ylmethyl)-9-oxa-2λ⁶-thia-3,5,12,19-tetrazatricyclo[12.3.1.14,8]nonadeca-1(18),4(19),5,7,14,16-hexaen-13-one (8.5 mg, 49%). ESI-MS m/z calc. 532.2144, found 533.4 (M+1)⁺; Retention time: 1.73 minutes; LC method A.

### Example 50: Preparation of Compound 50

### Step 1: Methyl 2-(tert-butoxycarbonylamino)-2-spiro[3.3]heptan-2-ylidene-acetate

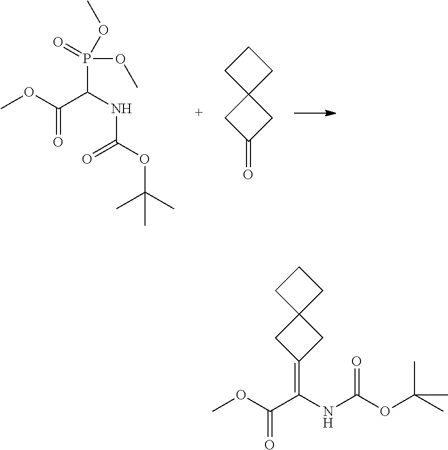 [SMILES: COC(=O)C(NC(=O)OC(C)(C)C)P(=O)(OC)OC ; O=C1CC2(CCC2)C1 ; COC(=O)C(NC(=O)OC(C)(C)C)=C1CC2(CCC2)C1]

To a solution of methyl 2-(tert-butoxycarbonylamino)-2-dimethoxyphosphoryl-acetate (17.55 g, 59.043 mmol) in ethyl acetate (175 mL) was added 1,1,3,3-tetramethylguanidine (9.1800 g, 10 mL, 79.703 mmol). The reaction mixture was stirred at room temperature for 35 minutes, followed by the addition of a solution of spiro[3.3]heptan-2-one (8.47 g, 76.892 mmol) in ethyl acetate (63 mL). The reaction mixture was stirred at room temperature for 6 days then quenched with the addition of a 1N aqueous solution of HCl (250 mL). The phases were separated, and the aqueous layer was extracted with ethyl acetate (3×200 mL). The combined organic layers were washed with a saturated solution of potassium bicarbonate (200 mL) and brine (200 mL) then dried over anhydrous sodium sulfate, filtered and concentrated under reduced pressure. The crude residue was purified by silica gel on a 330-g column, eluting from 0% to 10% ethyl acetate in heptanes, to afford methyl 2-(tert-butoxycarbonylamino)-2-spiro[3.3]heptan-2-ylidene-acetate (9.38 g, 52%) as a pale-yellow oil. ESI-MS m/z calc. 281.1627, found 226.2 (M−56)⁺; Retention time: 1.92 minutes. LC method X.

### Step 2: Methyl 2-(tert-butoxycarbonylamino)-2-spiro[3.3]heptan-2-yl-acetate

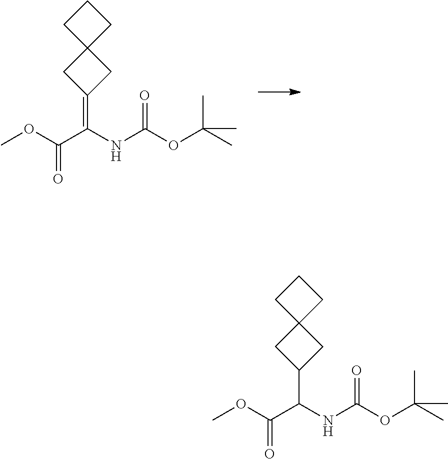 [SMILES: COC(=O)C(NC(=O)OC(C)(C)C)=C1CC2(CCC2)C1 ; COC(=O)C(NC(=O)OC(C)(C)C)C1CC2(CCC2)C1]

To a solution of methyl 2-(tert-butoxycarbonylamino)-2-spiro[3.3]heptan-2-ylidene-acetate (9.38 g, 30.973 mmol) in MeOH (185 mL) was added palladium on carbon (10%, 50% wet) (3.3 g, 5% w/w, 1.5505 mmol). The reaction was bubbled with nitrogen for 5 minutes then bubbled with hydrogen for 5 min. The reaction mixture was stirred at room temperature under 1 atm. of hydrogen for 2 hours. The reaction mixture was filtered on a Celite pad and the pad was rinsed with MeOH (10 mL). The crude residue was purified by silica gel on a 330-g column, eluting from 0% to 10% ethyl acetate in heptanes, to afford methyl 2-(tert-butoxycarbonylamino)-2-spiro[3.3]heptan-2-yl-acetate (7.04 g, 76%) as a colorless oil. ¹H NMR (400 MHz, CDCl₃) δ 4.91 (d, J 7.9 Hz, 1H), 4.19 (t, J 8.2 Hz, 1H), 3.71 (s, 3H), 2.47-2.33 (m, 1H), 2.07-1.94 (m, 4H), 1.91-1.84 (m, 3H), 1.82-1.73 (m, 3H), 1.44 (s, 9H). ESI-MS m/z calc. 283.1784, found 228.2 (M−56)⁺; Retention time: 1.97 minutes, LC method X.

### Step 3: tert-Butyl N-(2-hydroxy-1-spiro[3.3]heptan-2-yl-ethyl)carbamate

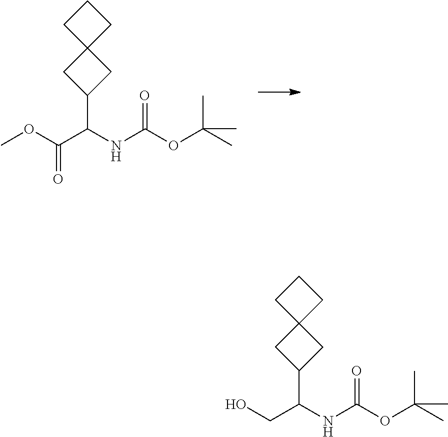 [SMILES: COC(=O)C(NC(=O)OC(C)(C)C)C1CC2(CCC2)C1 ; CC(C)(C)OC(=O)NC(CO)C1CC2(CCC2)C1]

To a solution of methyl 2-(tert-butoxycarbonylamino)-2-spiro[3.3]heptan-2-yl-acetate (7.04 g, 24.844 mmol) in THE (50 mL) was added a LiBH4 solution in THE (30 mL of 2 M, 60.000 mmol). The reaction mixture was stirred at room temperature for 4 hours then poured slowly over a saturated aqueous solution of ammonium chloride (100 mL) at 0° C. The product was extracted with EtOAc (3×50 mL). The combined organic layers were washed with brine (100 mL), dried with sodium sulfate, filtered and concentrated under reduced pressure to afford crude tert-butyl N-(2-hydroxy-1-spiro[3.3]heptan-2-yl-ethyl)carbamate (6.58 g, 99%) as a white solid. ¹H NMR (400 MHz, CDCl₃) δ 4.52 (br. s., 1H), 3.69-3.58 (m, 1H), 3.57-3.48 (m, 1H), 3.47-3.39 (m, 1H), 2.57 (br. s., 1H), 2.26-2.12 (m, 1H), 2.11-1.94 (m, 4H), 1.93-1.86 (m, 2H), 1.85-1.69 (m, 4H), 1.45 (s, 9H). ESI-MS m/z calc. 255.1834, found 200.2 (M−56)⁺; Retention time: 1.81 minutes, LC method X.

### Step 4: 2-Amino-2-spiro[3.3]heptan-2-yl-ethanol

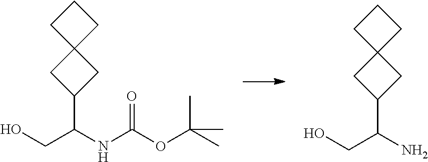 [SMILES: CC(C)(C)OC(=O)NC(CO)C1CC2(CCC2)C1 ; NC(CO)C1CC2(CCC2)C1]

To a solution of tert-butyl N-(2-hydroxy-1-spiro[3.3]heptan-2-yl-ethyl)carbamate (6.58 g, 25.768 mmol) in 1,4-dioxane (20 mL) was added a HCl solution in 1,4-dioxane (45 mL of 4 M, 180.00 mmol). The reaction mixture was stirred at room temperature for 20 hours then a HCl solution in 1,4-dioxane (20 mL of 4 M, 80.000 mmol) was again added. The reaction was stirred 4 more hours and the reaction was concentrated. The resulting solid was diluted in acetonitrile (1 mL) and water (5 mL) and was lyophilized to afford 2-amino-2-spiro[3.3]heptan-2-yl-ethanol (hydrochloride salt) (4.55 g, 88%) as a white solid. ¹H NMR (400 MHz, DMSO-d₆) δ 7.83 (br. s., 3H), 5.20 (t, J 5.0 Hz, 1H), 3.56-3.45 (m, 1H), 3.37-3.26 (m, 1H), 2.99-2.87 (m, 1H), 2.36-2.19 (m, 1H), 2.09-1.91 (m, 4H), 1.90-1.66 (m, 6H). ESI-MS m/z calc. 155.131, found 156.2 (M+1)⁺; Retention time: 1.64 minutes, LC method Y.

### Step 5: 3-[[4-(2-Amino-2-spiro[3.3]heptan-2-yl-ethoxy)-6-(2,6-dimethylphenyl)pyrimidin-2-yl]sulfamoyl]benzoic Acid

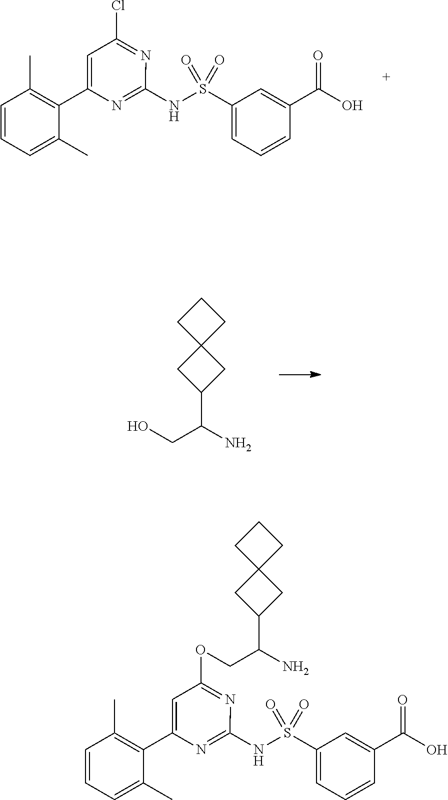 [SMILES: Cc1cccc(C)c1-c1cc(Cl)nc(NS(=O)(=O)c2cccc(C(=O)O)c2)n1 ; NC(CO)C1CC2(CCC2)C1 ; Cc1cccc(C)c1-c1cc(OCC(N)C2CC3(CCC3)C2)nc(NS(=O)(=O)c2cccc(C(=O)O)c2)n1]

To a solution of 2-amino-2-spiro[3.3]heptan-2-yl-ethanol (hydrochloride salt) (0.998 g, 5.2061 mmol) and 3-[[4-chloro-6-(2,6-dimethylphenyl)pyrimidin-2-yl]sulfamoyl]benzoic acid (hydrochloride salt) (2.375 g, 5.2275 mmol) in THE (12 mL) was added sodium tert-butoxide (2.507 g, 26.086 mmol). The reaction was stirred at room temperature for 30 minutes then THE (10 mL) and 2-methyl THE (5 mL) were added. The reaction was stirred for another 30 minutes then an aqueous solution of HCl 1N (10 mL) was added. The reaction was diluted with 2-methyl THE (20 mL) and the phase were separated. The aqueous layers were extracted with 2-methyl THE (3×20 mL) and the combined organic layers were dried over anhydrous sodium sulfate, filtered and concentrated under reduced pressure. The solid was diluted in EtOAc (100 mL) and stirred for 30 minutes then filtered on Buchner funnel. The solid was again diluted in EtOAc (100 mL) and stirred for 1 hour then filtered and dried under reduced pressure to afford 3-[[4-(2-amino-2-spiro[3.3]heptan-2-yl-ethoxy)-6-(2,6-dimethylphenyl)pyrimidin-2-yl]sulfamoyl]benzoic acid (hydrochloride salt) (2.24 g, 72%) as a white solid. ¹H NMR (400 MHz, DMSO-d₆) δ 8.44 (s, 1H), 8.27-8.06 (m, 5H), 7.70 (t, J=7.8 Hz, 1H), 7.31-7.21 (m, 1H), 7.13 (d, J 7.6 Hz, 2H), 6.30 (s, 1H), 4.28 (dd, J 11.6, 2.6 Hz, 1H), 4.15-3.97 (m, 1H), 3.50-3.34 (m, 1H), 2.43-2.25 (m, 1H), 2.14-1.70 (m, 16H). ESI-MS m/z calc. 536.2093, found 537.2 (M+1)⁺; Retention time: 2.49 minutes, LC method Y.

### Step 6: 6-(2,6-Dimethylphenyl)-2,2-dioxo-11-spiro[3.3]heptan-2-yl-9-oxa-2λ⁶-thia-3,5,12,19-tetrazatricyclo[12.3.1.14,8]nonadeca-1(18),4(19),5,7,14,16-hexaen-13-one (Compound 50)

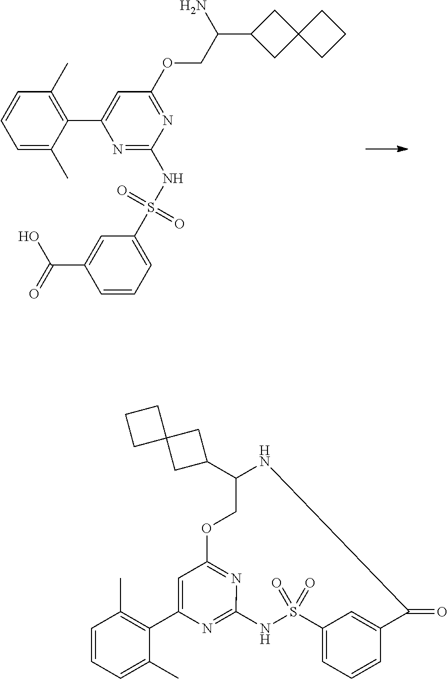 [SMILES: Cc1cccc(C)c1-c1cc(OCC(N)C2CC3(CCC3)C2)nc(NS(=O)(=O)c2cccc(C(=O)O)c2)n1 ; Cc1cccc(C)c1-c1cc2nc(n1)NS(=O)(=O)c1cccc(c1)C(=O)NC(C1CC3(CCC3)C1)CO2]

3-[[4-(2-amino-2-spiro[3.3]heptan-2-yl-ethoxy)-6-(2,6-dimethylphenyl)pyrimidin-2-yl]sulfamoyl]benzoic acid (50 mg, 0.09317 mmol) was combined with N-methylmorpholine (approximately 56.54 mg, 61.46 μL, 0.5590 mmol) in DMF (2 mL) and cooled to 0° C. CDMT (approximately 21.26 mg, 0.1211 mmol) was added and the reaction was warmed to room temperature after 30 minutes and allowed to stir for an additional hour. And the reaction mixture was then filtered and purified by reverse phase HPLC (1-99% ACN in water, HCl modifier, 15 min run) to give the corresponding 6-(2,6-dimethylphenyl)-2,2-dioxo-11-spiro[3.3]heptan-2-yl-9-oxa-2λ⁶-thia-3,5,12,19-tetrazatricyclo[12.3.1.14,8]nonadeca-1(18),4(19),5,7,14,16-hexaen-13-one (12.4 mg, 25%) upon drying of pure fractions.ESI-MS m/z calc. 518.1988, found 519.4 (M+1)⁺; Retention time: 1.64 minutes; LC method A.

### Example 51: Preparation of Compound 51

### Step 1: (2,3,6-Trimethylphenyl) trifluoromethanesulfonate

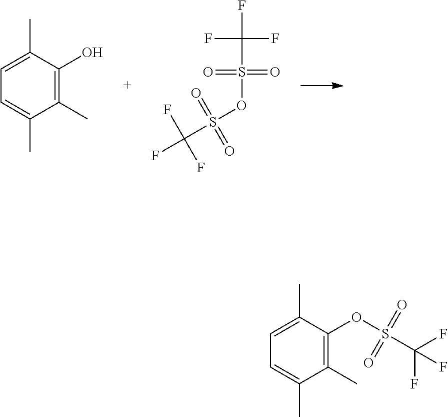

A solution of 2,3,6-trimethylphenol (5 g, 36.713 mmol) in dichloromethane (60 mL) was cooled down to 0° C. and triethylamine (4.4649 g, 6.15 mL, 44.124 mmol) was added. Then trifluoromethanesulfonic anhydride (12.443 g, 7.42 mL, 44.102 mmol) was added dropwise over 15 minutes. Upon addition, the ice bath was removed, and the mixture stirred at room temperature for 16 h. The mixture was diluted with dichloromethane (100 mL), washed with 1M hydrochloric solution (60 mL) and 5% aqueous sodium carbonate (2×50 mL) and brine (50 mL). The organic phase was dried over sodium sulfate, filtered and concentrated to dryness to give (2,3,6-trimethylphenyl) trifluoromethanesulfonate (8.9 g, 90%) as a brown oil. ¹H NMR (400 MHz, CDCl₃) δ 7.06 (d, J 7.8 Hz, 1H), 7.01 (d, J 7.8 Hz, 1H), 2.35 (s, 3H), 2.28 (s, 3H), 2.27 (s, 3H).

### Step 2: 5,5-Dimethyl-2-(2,3,6-trimethylphenyl)-1,3,2-dioxaborinane

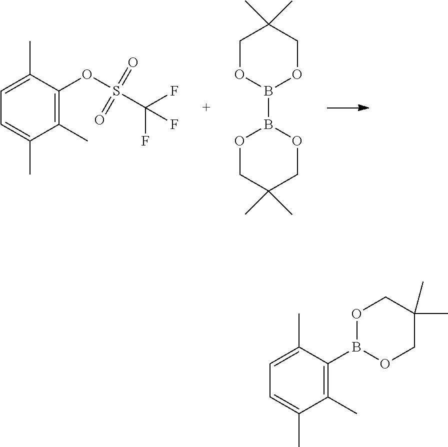 [SMILES: Cc1ccc(C)c(OS(=O)(=O)C(F)(F)F)c1C ; CC1(C)COB(B2OCC(C)(C)CO2)OC1 ; Cc1ccc(C)c(B2OCC(C)(C)CO2)c1C]

A solution of (2,3,6-trimethylphenyl) trifluoromethanesulfonate (8.2 g, 30.538 mmol), bis(neopentyl glycolato)diboron (20.75 g, 91.861 mmol) and potassium acetate (15 g, 152.84 mmol) in 1,4-dioxane (205 mL) was purged by bubbling nitrogen for 15 minutes. [1,1′-Bis(diphenylphosphino)ferrocene]dichloropalladium(II) (2.27 g, 3.1023 mmol) was added and the mixture was stirred for 18 hours at 100-105° C. The mixture was filtered, adsorbed on silica and the product was purified by two successive flash chromatography purifications (on silica 120 g) eluting with 0% to 10% ethyl acetate in heptane to afford 5,5-dimethyl-2-(2,3,6-trimethylphenyl)-1,3,2-dioxaborinane (5.42 g, 72%) as a yellow oil. ¹H NMR (400 MHz, CDCl₃) δ 7.00 (d, J 7.8 Hz, 1H), 6.89 (d, J 7.6 Hz, 1H), 3.82 (s, 4H), 2.37 (s, 3H), 2.31 (s, 3H), 2.22 (s, 3H), 1.14 (s, 6H). ESI-MS m/z calc. 232.1635, found 233.2 (M+1)⁺; Retention time: 4.81 minutes, LC method Y.

### Step 3: tert-Butyl N-[4-chloro-6-(2,3,6-trimethylphenyl)pyrimidin-2-yl]carbamate

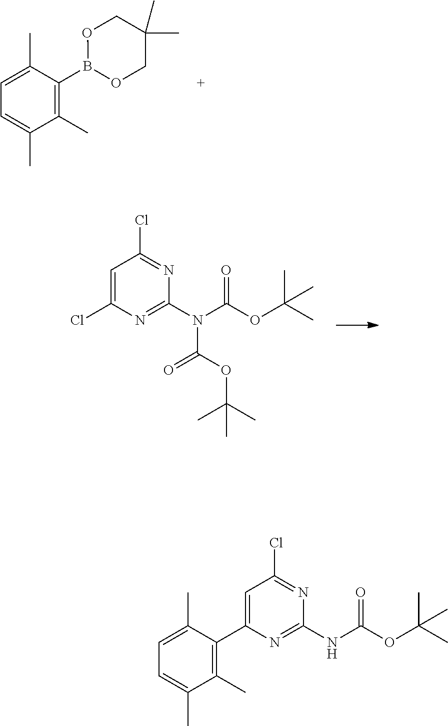 [SMILES: Cc1ccc(C)c(B2OCC(C)(C)CO2)c1C ; CC(C)(C)OC(=O)N(C(=O)OC(C)(C)C)c1nc(Cl)cc(Cl)n1 ; Cc1ccc(C)c(-c2cc(Cl)nc(NC(=O)OC(C)(C)C)n2)c1C]

5,5-Dimethyl-2-(2,3,6-trimethylphenyl)-1,3,2-dioxaborinane (1.00 g, 4.308 mmol) was combined with tert-butyl N-tert-butoxycarbonyl-N-(4,6-dichloropyrimidin-2-yl)carbamate (1.88 g, 5.162 mmol) and dissolved in 1,4-dioxane (17 mL). Water (3 mL) was added followed by barium hydroxide octahydrate (4 g, 12.68 mmol). Pd(dppf)Cl₂ (176 mg, 0.2155 mmol) was added last under nitrogen gas. The reaction mixture was allowed to stir at 80° C. for 1 hour. The reaction mixture was diluted with EtOAc (100 mL) and washed with aqueous HCl (0.5 M, 1×100 mL). The aqueous layer was extracted with EtOAc (1×100 mL). All organic layers were combined and washed with brine (1×75 mL), dried over sodium sulfate, filtered and concentrated under reduced pressure. The crude product was purified by flash chromatography on a 80 gram silica gel column eluting with a 0-30% EtOAc/hexane gradient over 40 minutes; to give tert-butyl N-[4-chloro-6-(2,3,6-trimethylphenyl)pyrimidin-2-yl]carbamate (1.57 g, 105%) as a clear colorless oil. ESI-MS m/z calc. 347.14005, found 292.3 (M−55)⁺; Retention time: 2.04 minutes, LC method A.

### Step 4: 4-Chloro-6-(2,3,6-trimethylphenyl)pyrimidin-2-amine

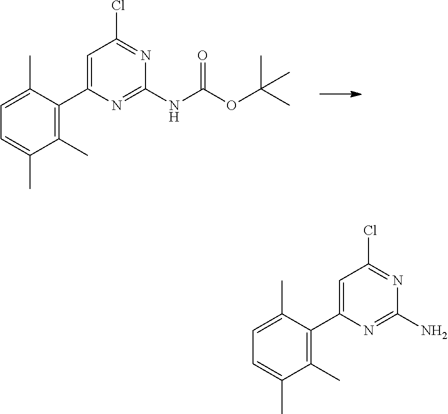 [SMILES: Cc1ccc(C)c(-c2cc(Cl)nc(NC(=O)OC(C)(C)C)n2)c1C ; Cc1ccc(C)c(-c2cc(Cl)nc(N)n2)c1C]

tert-Butyl N-[4-chloro-6-(2,3,6-trimethylphenyl)pyrimidin-2-yl]carbamate (1.57 g, 4.514 mmol) was dissolved in dichloromethane (8 mL). A solution of HCl (5 mL of 4 M, 20.00 mmol) in dioxane was added. The reaction mixture was allowed to stir at room temperature overnight. The obtained slurry was diluted with dichloromethane (75 mL) and washed with aqueous NaOH (1 M, 1×75 mL). The aqueous layer was extracted with dichloromethane (1×75 mL). The organic layers were combined and washed with water (1×100 mL). The organic layer was then dried over sodium sulfate, filtered and concentrated under reduced pressure to give 4-chloro-6-(2,3,6-trimethylphenyl)pyrimidin-2-amine (1.06 g, 95%) as a white waxy semi-solid. ESI-MS m/z calc. 247.08763, found 248.1 (M+1)⁺; Retention time: 1.54 minutes, LC method A.

### Step 5: Methyl 3-[[4-chloro-6-(2,3,6-trimethylphenyl)pyrimidin-2-yl]sulfamoyl]benzoate

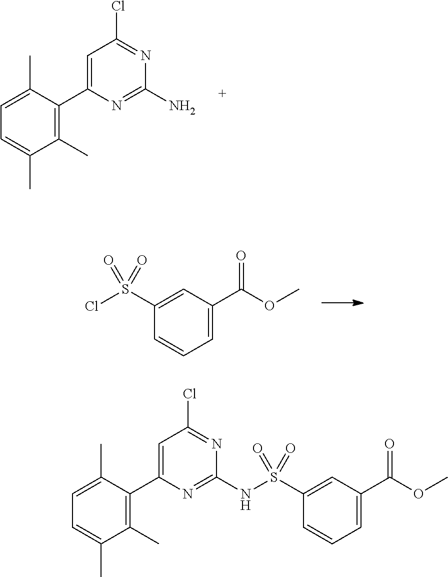

4-Chloro-6-(2,3,6-trimethylphenyl)pyrimidin-2-amine (1.06 g, 4.279 mmol) was dissolved in tetrahydrofuran (21 mL) and cooled to 0° C. before the addition of sodium hydride (428 mg, 10.70 mmol) (60 wt % dispersion in mineral oil). After stirring for 5 minutes, methyl 3-chlorosulfonylbenzoate (1.51 g, 6.435 mmol) was slowly added dropwise. The reaction mixture was allowed to stir at room temperature for 2 hours. Aqueous HCl (1 M, 75 mL) was added, and the resulting mixture was extracted with EtOAc (2×75 mL). The combined organic layers were washed with brine (1×100 mL), dried over sodium sulfate, filtered and concentrated under reduced pressure. The crude product was chromatographed on a 40 gram silica gel column eluting with a 0-35% EtOAc/hexane gradient over 40 minutes to give methyl 3-[[4-chloro-6-(2,3,6-trimethylphenyl)pyrimidin-2-yl]sulfamoyl]benzoate (631 mg, 33%) as a white solid. ESI-MS m/z calc. 445.0863, found 446.1 (M+1)⁺; Retention time: 1.86 minutes, LC method A.

### Step 6: 3-[[4-Chloro-6-(2,3,6-trimethylphenyl)pyrimidin-2-yl]sulfamoyl]benzoic Acid

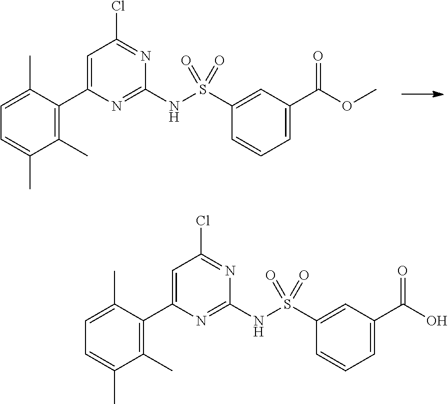 [SMILES: COC(=O)c1cccc(S(=O)(=O)Nc2nc(Cl)cc(-c3c(C)ccc(C)c3C)n2)c1 ; Cc1ccc(C)c(-c2cc(Cl)nc(NS(=O)(=O)c3cccc(C(=O)O)c3)n2)c1C]

Methyl 3-[[4-chloro-6-(2,3,6-trimethylphenyl)pyrimidin-2-yl]sulfamoyl]benzoate (330 mg, 0.7400 mmol) was dissolved in tetrahydrofuran (2.8 mL) and cooled to 0° C. An aqueous solution of sodium hydroxide (1.0 mL of 3 M, 3.000 mmol) was added, and the reaction mixture was allowed to stir at 0° C. for 2 hours. The reaction mixture was diluted with aqueous HCl (1 M, 75 mL) and extracted with EtOAc (2×75 mL). The combined organic layers were washed with brine (1×100 mL), dried over sodium sulfate, filtered and concentrated under reduced pressure. 3-[[4-chloro-6-(2,3,6-trimethylphenyl)pyrimidin-2-yl]sulfamoyl]benzoic Acid (317 mg, 99%) was obtained as a foaming solid. ¹H NMR (400 MHz, DMSO-d₆) δ 13.39 (s, 1H), 12.42 (s, 1H), 8.42 (t, J 1.9 Hz, 1H), 8.18 (dt, J 7.8, 1.5 Hz, 1H), 8.11 (dt, J 8.0, 1.4 Hz, 1H), 7.68 (t, J 7.8 Hz, 1H), 7.24 (s, 1H), 7.13 (d, J 7.7 Hz, 1H), 6.98 (d, J 7.7 Hz, 1H), 2.20 (s, 3H), 1.75 (s, 3H), 1.67 (s, 3H). ESI-MS m/z calc. 431.07065, found 432.1 (M+1)⁺; Retention time: 1.62 minutes, LC method A.

### Step 7: 3-[[4-[(2R)-2-Amino-4,4-dimethyl-pentoxy]-6-(2,3,6-trimethylphenyl)pyrimidin-2-yl]sulfamoyl]benzoic Acid

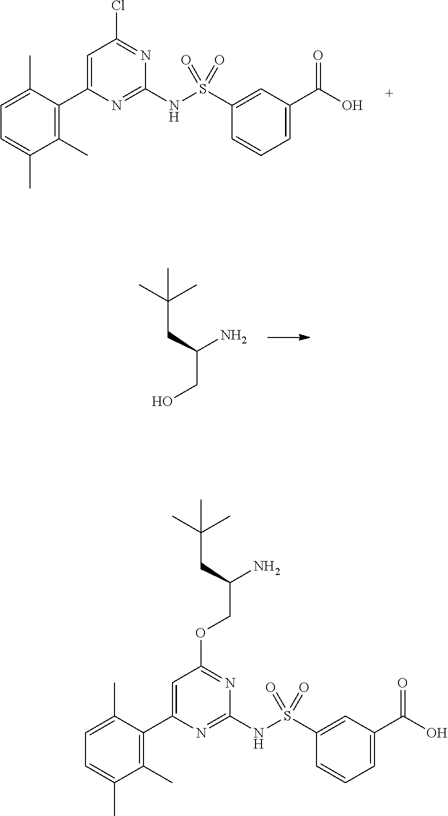

3-[[4-Chloro-6-(2,3,6-trimethylphenyl)pyrimidin-2-yl]sulfamoyl]benzoic acid (317 mg, 0.7340 mmol) and (2R)-2-amino-4,4-dimethyl-pentan-1-ol (hydrochloride salt) (135 mg, 0.8051 mmol) were combined and dissolved/suspended in tetrahydrofuran (5.0 mL). Solid sodium tert-butoxide (353 mg, 3.673 mmol) was added in gradual portions over 2 minutes. The reaction mixture was allowed to stir at room temperature for 2 hours. The reaction mixture was diluted with EtOAc (75 mL). It was then washed with aqueous HCl (0.5 M, 1×75 mL) and brine (1×75 mL). The organic layer was dried over sodium sulfate, filtered and concentrated under reduced pressure. The product was isolated by chromatography on a 12-gram silica gel column eluting with a 0-100% methanol/dichloromethane gradient over 16 minutes. Fractions containing the desired product were combined with HCl (190 μL of 4 M, 0.7600 mmol) and concentrated under reduced pressure to give 3-[[4-[(2R)-2-amino-4,4-dimethyl-pentoxy]-6-(2,3,6-trimethylphenyl)pyrimidin-2-yl]sulfamoyl]benzoic acid (hydrochloride salt) (223 mg, 54%) as a white solid. ESI-MS m/z calc. 526.225, found 527.3 (M+1)⁺; Retention time: 1.14 minutes, LC method A.

### Step 8: (11R)-11-(2,2-Dimethylpropyl)-6-(2,3,6-trimethylphenyl)-9-oxa-2λ⁶-thia-3,5,12,19-tetraazatricyclo[12.3.1.14,8]nonadeca-1(18),4,6,8(19),14,16-hexaene-2,2,13-trione (Compound 51)

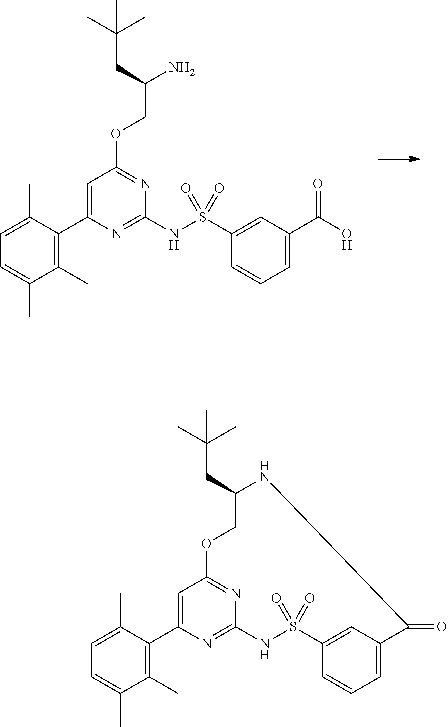 [SMILES: Cc1ccc(C)c(-c2cc(OC[C@H](N)CC(C)(C)C)nc(NS(=O)(=O)c3cccc(C(=O)O)c3)n2)c1C ; Cc1ccc(C)c(-c2cc3nc(n2)NS(=O)(=O)c2cccc(c2)C(=O)N[C@H](CC(C)(C)C)CO3)c1C]

3-[[4-[(2R)-2-Amino-4,4-dimethyl-pentoxy]-6-(2,3,6-trimethylphenyl)pyrimidin-2-yl]sulfamoyl]benzoic acid (hydrochloride salt) (40 mg, 0.07103 mmol) was dissolved in DMF (3 mL). N-methylmorpholine (50 μL, 0.4548 mmol) was added, and CDMT (16 mg, 0.09113 mmol) was added at 0° C. The reaction mixture was allowed to stir overnight at room temperature. The product was purified by reverse-phase HPLC eluting with a 10-99% acetonitrile/water gradient over 30 minutes with 0.5 mM acid modifier in the aqueous phase, to give (11R)-11-(2,2-dimethylpropyl)-6-(2,3,6-trimethylphenyl)-9-oxa-2λ⁶-thia-3,5,12,19-tetraazatricyclo[12.3.1.14,8]nonadeca-1(18),4,6,8(19),14,16-hexaene-2,2,13-trione (11.1 mg, 31%) as a white solid. ESI-MS m/z calc. 508.21442, found 509.3 (M+1)⁺; Retention time: 1.61 minutes, LC method A.

### Example 52: Preparation of Compound 52

### Step 1: tert-Butyl N-tert-butoxycarbonyl-N-[4-chloro-6-(2-methyl-1-naphthyl)pyrimidin-2-yl]carbamate

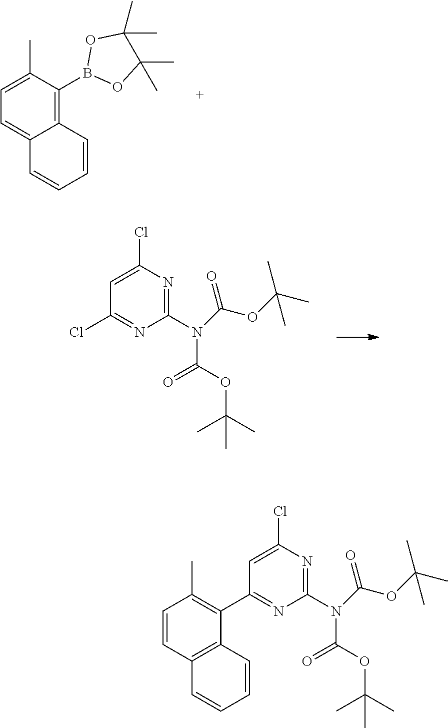 [SMILES: Cc1ccc2ccccc2c1B1OC(C)(C)C(C)(C)O1 ; CC(C)(C)OC(=O)N(C(=O)OC(C)(C)C)c1nc(Cl)cc(Cl)n1 ; Cc1ccc2ccccc2c1-c1cc(Cl)nc(N(C(=O)OC(C)(C)C)C(=O)OC(C)(C)C)n1]

A solution of, 4,4,5,5-tetramethyl-2-(2-methyl-1-naphthyl)-1,3,2-dioxaborolane (2.03 g, 7.4188 mmol), tert-butyl N-tert-butoxycarbonyl-N-(4,6-dichloropyrimidin-2-yl)carbamate (4.651 g, 11.493 mmol) and Cesium carbonate (6.064 g, 18.612 mmol) in a mixture of DME (30 mL) and water (10 mL) was degassed with nitrogen for 5 minutes before adding Pd(dppf)Cl₂ (525.5 mg, 0.7182 mmol) and degassing for another 5 min under nitrogen. The mixture was then stirred at 80° C. for 1 hour. The mixture was then partitioned with DI water (50 mL) and EtOAc (150 mL). The aqueous layer was extracted with EtOAc (2×100 mL). The organic layers were combined, washed with brine, dried over anhydrous sodium sulfate, filtered, and concentrated under reduced pressure. The crude was purified via silica gel column chromatography (load silica gel) (40 g column, eluting 0 to 15% EtOAc in Hexanes) yielding tert-butyl N-tert-butoxycarbonyl-N-[4-chloro-6-(2-methyl-1-naphthyl)pyrimidin-2-yl]carbamate (3.177 g, 73%) as a yellow solid. ESI-MS m/z calc. 469.1768, found 470.2 (M+1)⁺; Retention time: 4.06 minutes, LC method T.

### Step 2: 4-Chloro-6-(2-methyl-1-naphthyl)pyrimidin-2-amine

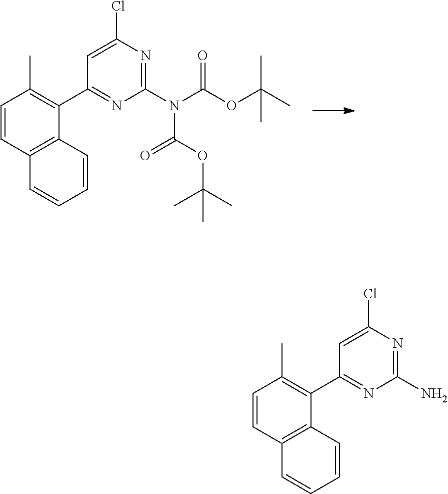 [SMILES: Cc1ccc2ccccc2c1-c1cc(Cl)nc(N(C(=O)OC(C)(C)C)C(=O)OC(C)(C)C)n1 ; Cc1ccc2ccccc2c1-c1cc(Cl)nc(N)n1]

To a solution of tert-butyl N-tert-butoxycarbonyl-N-[4-chloro-6-(2-methyl-1-naphthyl)pyrimidin-2-yl]carbamate (3.342 g, 5.6890 mmol) in DCM (20 mL) at 0° C. was added HCl in dioxane (20 mL of 4 M, 80.000 mmol). The reaction was then raised to RT and stirred for 3 hours. The reaction was then quenched with aqueous sodium bicarbonate (150 mL) and DCM (100 mL). The aqueous layer was extracted with DCM (2×100 mL). The organic layers were combined, washed with brine, dried over anhydrous sodium sulfate, filtered, and concentrated under reduced pressure to yield 4-chloro-6-(2-methyl-1-naphthyl)pyrimidin-2-amine (2.15 g, 130%) as a yellow solid. ESI-MS m/z calc. 269.072, found 270.0 (M+1)⁺; Retention time: 2.97 minutes, LC method T.

### Step 3: Methyl 3-[[4-chloro-6-(2-methyl-1-naphthyl)pyrimidin-2-yl]sulfamoyl]benzoate

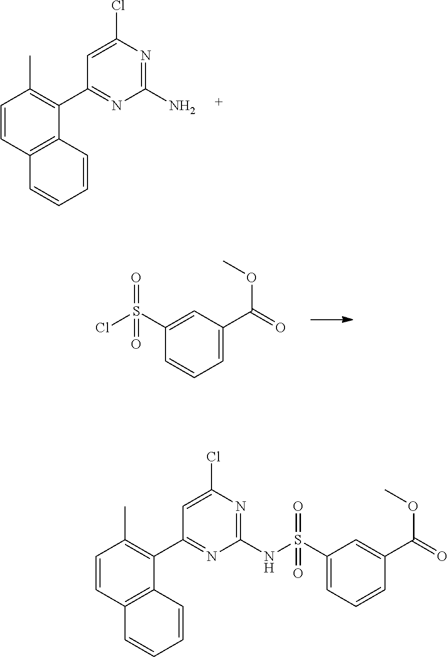 [SMILES: Cc1ccc2ccccc2c1-c1cc(Cl)nc(N)n1 ; COC(=O)c1cccc(S(=O)(=O)Cl)c1 ; COC(=O)c1cccc(S(=O)(=O)Nc2nc(Cl)cc(-c3c(C)ccc4ccccc34)n2)c1]

A solution of crude 4-chloro-6-(2-methyl-1-naphthyl)pyrimidin-2-amine (2.15 g, 7.4130 mmol) in anhydrous THE (28 mL) was cooled to 0° C. Then a solution of methyl 3-chlorosulfonylbenzoate (2.278 g, 9.7078 mmol) in anhydrous THE (35 mL) was added. Lithium tert-amoxide in heptane (1.3724 g, 4.7 mL of 40% w/w, 5.8350 mmol) was then added dropwise. The reaction was brought up to room temperature and stirred for 2 hours. The reaction was then quenched with 1M HCl (50 mL) and EtOAc (100 mL). The aqueous layer was extracted with EtOAc (2×100 mL). The organic layers were combined, washed with brine, dried over anhydrous sodium sulfate, filtered, and concentrated under reduced pressure. The crude was purified via silica gel column chromatography (load silica gel) (40 g column, eluting 0 to 35% EtOAc in Hexanes) to yield methyl 3-[[4-chloro-6-(2-methyl-1-naphthyl)pyrimidin-2-yl]sulfamoyl]benzoate (2.579 g, 64%) as a white solid. ESI-MS m/z calc. 467.0707, found 468.1 (M+1)⁺; Retention time: 3.39 minutes, LC method T.

### Step 4: 3-[[4-Chloro-6-(2-methyl-1-naphthyl)pyrimidin-2-yl]sulfamoyl]benzoic Acid

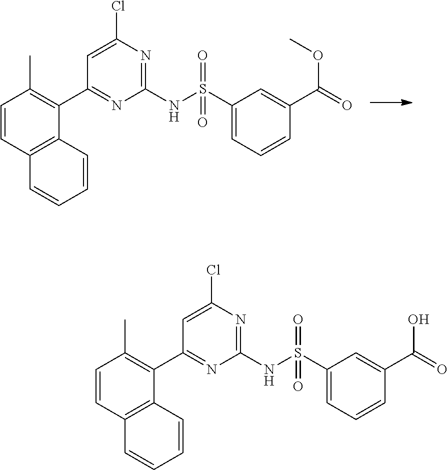 [SMILES: COC(=O)c1cccc(S(=O)(=O)Nc2nc(Cl)cc(-c3c(C)ccc4ccccc34)n2)c1 ; Cc1ccc2ccccc2c1-c1cc(Cl)nc(NS(=O)(=O)c2cccc(C(=O)O)c2)n1]

To a solution of methyl 3-[[4-chloro-6-(2-methyl-1-naphthyl)pyrimidin-2-yl]sulfamoyl]benzoate (2.554 g, 5.4581 mmol) in THE (51 mL) was added aqueous solution of NaOH (11 mL of 2 M, 22.000 mmol). The solution was stirred for 1 hour. The solution was then quenched with 1M HCl (10 mL) and EtOAc (20 mL). The aqueous layer was then extracted with EtOAc (2×20 mL). The organic layers were combined, washed with brine, dried over anhydrous sodium sulfate, filtered, and concentrated under reduced pressure to yield 3-[[4-chloro-6-(2-methyl-1-naphthyl)pyrimidin-2-yl]sulfamoyl]benzoic acid (2.439 g, 87%) as a white solid. ESI-MS m/z calc. 453.055, found 454.0 (M+1)⁺; Retention time: 3.03 minutes, LC method T.

### Step 5: 3-[[4-[(2R)-2-Amino-4,4-dimethyl-pentoxy]-6-(2-methyl-1-naphthyl)pyrimidin-2-yl]sulfamoyl]benzoic Acid

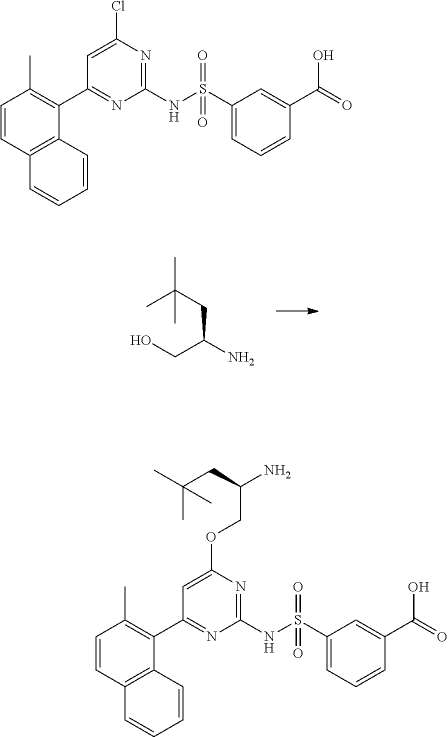

To a solution of 3-[[4-chloro-6-(2-methyl-1-naphthyl)pyrimidin-2-yl]sulfamoyl]benzoic acid (2.412 g, 5.3140 mmol) and (2R)-2-amino-4,4-dimethyl-pentan-1-ol (hydrochloride salt) (1.023 g, 6.1010 mmol) in anhydrous THE (85 mL) was added sodium tert-butoxide (2.054 g, 21.373 mmol). The solution was stirred at RT for 2 hours. The solution was then concentrated under reduced pressure. The residue was then purified via reverse phase HPLC (gradient 25-75% acetonitrile in water buffered by 5 mM HCl) to yield 3-[[4-[(2R)-2-amino-4,4-dimethyl-pentoxy]-6-(2-methyl-1-naphthyl)pyrimidin-2-yl]sulfamoyl]benzoic Acid (hydrochloride salt) (1.345 g, 41%) as a white powder. ESI-MS m/z calc. 548.2093, found 549.4 (M+1)⁺; Retention time: 1.88 minutes, LC method W. ¹H NMR (500 MHz, DMSO-d₆) δ 13.43 (s, 1H), 8.48 (d, J 1.8 Hz, 1H), 8.27-8.10 (m, 5H), 7.97 (m, 2H), 7.70 (m, 1H), 7.54-7.42 (m, 3H), 6.43 (s, 1H), 4.37 (d, J 11.8 Hz, 1H), 4.16 (m, 1H), 3.60 (s, 1H), 2.21 (s, 3H), 1.65-1.59 (m, 1H), 1.52 (m, 1H), 0.96 (s, 9H).

### Step 6: (11R)-11-(2,2-Dimethylpropyl)-6-(2-methyl-1-naphthyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 52)

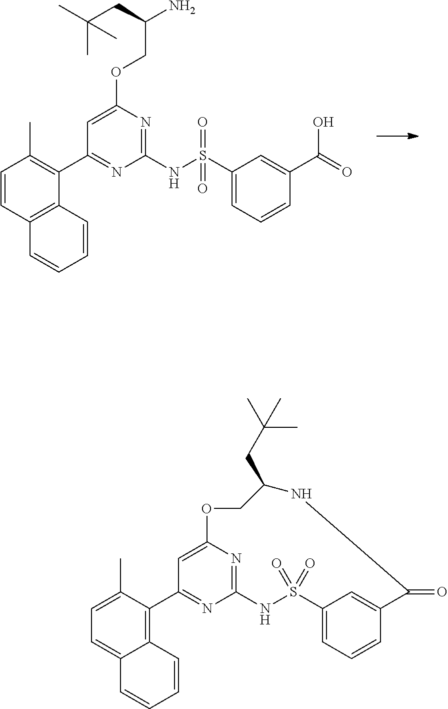 [SMILES: Cc1ccc2ccccc2c1-c1cc(OC[C@H](N)CC(C)(C)C)nc(NS(=O)(=O)c2cccc(C(=O)O)c2)n1 ; Cc1ccc2ccccc2c1-c1cc2nc(n1)NS(=O)(=O)c1cccc(c1)C(=O)N[C@H](CC(C)(C)C)CO2]

3-[[4-[(2R)-2-amino-4,4-dimethyl-pentoxy]-6-(2-methyl-1-naphthyl)pyrimidin-2-yl]sulfamoyl]benzoic acid (hydrochloride salt) (26.2 mg, 0.04478 mmol), HATU (19.1 mg, 0.05023 mmol), and triethylamine (100 μL, 0.7175 mmol) were combined in DMF (1 mL) and stirred at room temperature for 2 h. The reaction mixture was filtered and purified by reverse-phase HPLC utilizing a gradient of 1-99% acetonitrile in 5 mM aqueous HCl to yield (11R)-11-(2,2-dimethylpropyl)-6-(2-methyl-1-naphthyl)-2,2-dioxo-9-oxa-2λ⁶-thia-3,5,12,19-tetrazatricyclo[12.3.1.14,8]nonadeca-1(18),4(19),5,7,14,16-hexaen-13-one (6 mg, 25%) as a white solid. ESI-MS m/z calc. 530.1988, found 531.4 (M+1)⁺; Retention time: 1.63 minutes, LC method A.

### Example 53: Preparation of Compound 53

### Step 1: (2-Bromo-3-methyl-phenyl)methanol

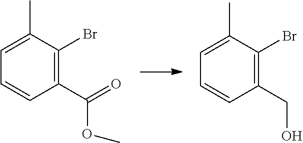

To a solution of methyl 2-bromo-3-methyl-benzoate (60.00 g, 256.69 mmol) in anhydrous THE (600 mL) stirring at internal temperature of 0° C. was added Lithium Borohydride (29.51 g, 1.2869 mol) portionwise. The reaction mixture was then heated to and stirred at internal temperature of 50° C. for 4 h. The reaction was cooled to 0° C. and was slowly quenched with DI water (300 mL). The mixture was extracted with EtOAc (3×250 mL). The combined EtOAc layers were washed with saturated aqueous NaCl (300 mL), dried over anhydrous sodium sulfate, filtered and concentrated under vacuum. The crude product (49.19 g) was obtained as a light orange solid. (2-bromo-3-methyl-phenyl)methanol (49.19 g, 95%)¹H NMR (500 MHz, DMSO-d₆) δ 7.36 (d, J 7.5 Hz, 1H), 7.28 (t, J 7.5, 7.5 Hz, 1H), 7.24 (d, J 7.9 Hz, 1H), 5.40 (t, J 5.6, 5.6 Hz, 1H), 4.50 (d, J 5.8 Hz, 2H), 2.35 (s, 3H). ESI-MS m/z calc. 199.98367, Retention time: 2.32 minutes; LC method T.

### Step 2: 2-Bromo-3-methyl-benzaldehyde

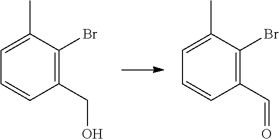

To a solution of (2-bromo-3-methyl-phenyl)methanol (49.19 g, 244.65 mmol) and TEMPO (3.92 g, 24.587 mmol) in DCM (260 mL) stirring at RT was added PhI(OAc)2 (99.61 g, 293.79 mmol). The reaction mixture was stirred for 2 h. Aqueous saturated solution of sodium thiosulfate (250 mL) was added and stirred for 0.5 h. The bilayers were separated, and the aqueous layer was extracted with DCM (2×250 mL). The combined DCM layers were washed with saturated aqueous sodium bicarbonate (250 mL), dried over anhydrous sodium sulfate, filtered and concentrated under vacuum. Crude Mass=112.57 g (Orange Oil). The crude was subjected to flash chromatography (Loaded in DCM) (330 g silica gel, eluting 0 to 30% EtOAc/Hexanes in 75 min., Flow Rate=80 mL/min., Detection=UV254). Appropriate fractions were collected and concentrated under vacuum. Final Product (47.87 g) was obtained as an Off-White Solid. 2-bromo-3-methyl-benzaldehyde (47.87 g, 98%)¹H NMR (500 MHz, Chloroform-d) δ 10.46 (s, 1H), 7.74 (dm, 1H), 7.48 (dm, J 7.4, 1.7, 0.8, 0.8 Hz, 1H), 7.32 (t, J 7.5, 7.5 Hz, 1H), 2.48 (s, 3H). ESI-MS m/z calc. 197.96803, Retention time: 2.76 minutes; LC method T.

### Step 3: 2-Bromo-1-[(E)-2-cyclopropylvinyl]-3-methyl-benzene

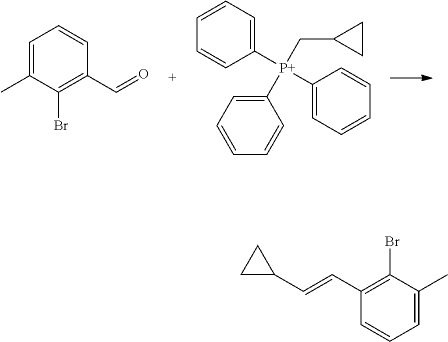

In a 20 mL vial potassium tert-butoxide (260 mg, 2.317 mmol) was dissolved in THE (2.0 mL) and added to a suspension of cyclopropylmethyl(triphenyl)phosphonium bromide (900 mg, 2.265 mmol) in THE (2.0 mL) at 0° C. and then the mixture was warmed up to rt for 1 h. The reaction mixture was cooled back to 0° C. and a solution of 2-bromo-3-methyl-benzaldehyde (200 mg, 1.005 mmol) in THE (2.0 mL) was added. The resulting reaction mixture was warmed to rt over a period of 12 h. The reaction mixture was quenched with cold water and extracted with dichloromethane. The combined organic layers were washed with brine, dried over sodium sulfate, filtered and concentrated. The mixture was then purified on silica gel chromatography (24 gram column) using a gradient from 100% hexanes to 30% ethyl acetate in hexanes to afford 2-bromo-1-[(E)-2-cyclopropylvinyl]-3-methyl-benzene (206 mg, 86%) ESI-MS m/z calc. 236.02007, found 237.2 (M+1)⁺; Retention time: 2.17 minutes. ¹H NMR (500 MHz, DMSO-d₆) δ 7.41-7.33 (m, 1H), 7.31-7.19 (m, 1H), 7.19-7.12 (m, 1H), 6.82-6.29 (m, 1H), 5.80-5.11 (m, 1H), 2.37 (d, J 13.3 Hz, 3H), 1.64 (dtd, J 19.5, 10.6, 9.4, 4.0 Hz, 1H), 0.89-0.75 (m, 2H), 0.51 (ddt, J=33.5, 6.2, 4.2 Hz, 2H), LC method A.

### Step 4: (11R)-6-Chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one

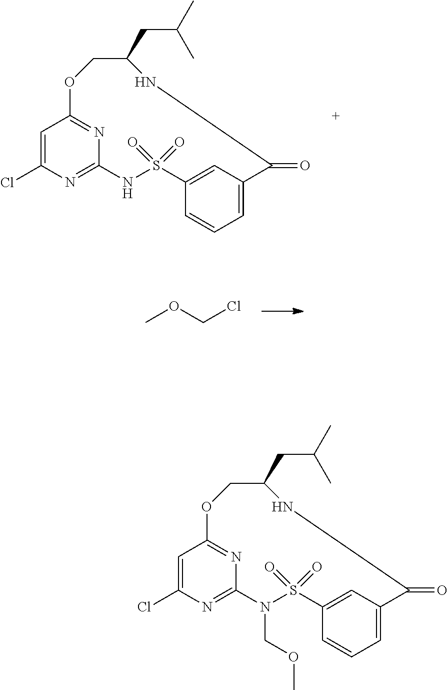

(11R)-6-Chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (1 g, 2.434 mmol) was dissolved in acetonitrile (15.0 mL). DCE (15.0 mL). The mixture was treated with powdered Potassium carbonate (510 mg, 3.690 mmol) and chloro(methoxy)methane (215 μL, 2.831 mmol). The mixture was stirred at 20° C. for 16 h. The reaction mixture was filtered and concentrated. The crude product was purified by silica gel chromatography using a 40 g column eluting with 100% hexanes to 90% ethyl acetate in hexanes to afford a white solid (11R)-6-chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one (860 mg, 78%) ESI-MS m/z calc. 454.10776, found 455.2 (M+1)⁺; Retention time: 1.69 minutes (LC method A). ¹H NMR (500 MHz, DMSO-d₆) δ 8.58 (s, 1H), 8.10 (d, J 7.8 Hz, 1H), 7.95 (d, J 9.7 Hz, 1H), 7.88-7.71 (m, 2H), 6.95 (s, 1H), 5.61 (d, J 11.0 Hz, 1H), 5.53 (d, J 11.0 Hz, 1H), 5.06 (dd, J=11.3, 4.0 Hz, 1H), 3.87 (t, J=11.3 Hz, 1H), 3.22 (d, J=13.6 Hz, 1H), 3.10 (s, 3H), 1.60-1.42 (m, 2H), 1.20 (dd, J 13.4, 10.6 Hz, 1H), 0.78 (d, J 6.6 Hz, 3H), 0.28 (d, J 6.4 Hz, 3H).

### Step 5: 2-[2-[(E)-2-Cyclopropylvinyl]-6-methyl-phenyl]-4,4,5,5-tetramethyl-1,3,2-dioxaborolane

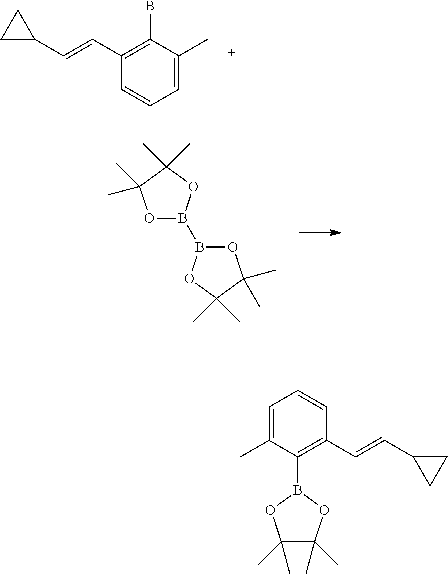

In a 250 mL round bottom flask was dissolved 2-bromo-1-[(E)-2-cyclopropylvinyl]-3-methyl-benzene (206 mg, 0.8687 mmol) in dioxane (2.5 mL) and to it was added KOAc (185 mg, 1.885 mmol) and the mixture was degassed with nitrogen for several minutes. Then bis(pinacol)diboron (345 mg, 1.359 mmol) was added, followed by cyclopenta-1,4-dien-1-yl(diphenyl)phosphane; dichloropalladium; iron (70 mg, 0.09593 mmol) and the reaction was purged again by N₂, sealed and heated to 100° C. for 16 hours. After the reaction was cooled to room temperature, saturated ammonium chloride was added and extracted with ethyl acetate. The combined organic extracts washed with brine, dried over sodium sulfate, filtered and concentrated. The resulting brown oil was purified utilizing silica gel column chromatography (24 gram column) using a gradient of 100% hexanes to 30% ethyl acetate in hexanes to obtain the desired compound as a white solid 2-[2-[(E)-2-cyclopropylvinyl]-6-methyl-phenyl]-4,4,5,5-tetramethyl-1,3,2-dioxaborolane (102 mg, 41%) ESI-MS m/z calc. 284.19476, found 285.2 (M+1)⁺; Retention time: 2.19 minutes, LC method A.

### Step 6: (11R)-6-[2-[(E)-2-Cyclopropylvinyl]-6-methyl-phenyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 53)

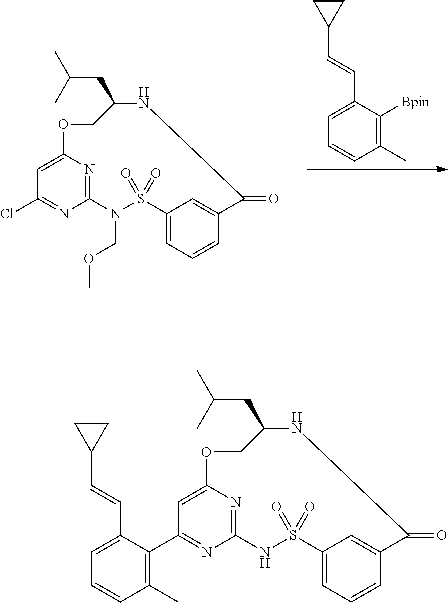

A heterogeneous mixture of (11R)-6-chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one (130 mg, 0.2858 mmol), 2-[2-[(E)-2-cyclopropylvinyl]-6-methyl-phenyl]-4,4,5,5-tetramethyl-1,3,2-dioxaborolane (98 mg, 0.3448 mmol), Pd(dppf)Cl₂ (48 mg, 0.05878 mmol) and potassium carbonate (120 mg, 0.8683 mmol) in DMA (3.5 mL) was heated for 2 h at 105° C. in a sealed microwave tube. The mixture was cooled to ambient temperature, filtered and then TFA (500 μL, 6.490 mmol) was added and the mixture was stirred for 14 h. The crude material was filtered and concentrated under a stream of nitrogen to give a residue. This mixture was purified by reverse-phase preparative chromatography utilizing a C₁₈ column (1-99% gradient of acetonitrile in water+5 mM HCl, 30 minute method) to afford as a tan solid (11R)-6-[2-[(E)-2-cyclopropylvinyl]-6-methyl-phenyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (21.6 mg, 14%) ESI-MS m/z calc. 532.2144, found 533.2 (M+1)⁺; Retention time: 1.7 minutes and the side product (11R)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (8.61 mg, 8%) ESI-MS m/z calc. 376.1205, found 377.2 (M+1)⁺; Retention time: 0.87 minutes. ¹H NMR (500 MHz, DMSO-d₆) δ 12.84 (s, 1H), 8.41 (s, 1H), 8.09 (d, J 6.8 Hz, 1H), 7.89-7.84 (m, 2H), 7.62 (d, J=7.3 Hz, 2H), 6.34 (d, J 6.8 Hz, 1H), 5.13 (dd, J 11.0, 3.8 Hz, 1H), 3.83 (t, J 11.0 Hz, 1H), 3.16 (d, J 12.7 Hz, 1H), 1.55-1.47 (m, 2H), 1.17 (t, J 12.0 Hz, 1H), 0.79 (d, J 6.6 Hz, 3H), 0.30 (d, J 6.4 Hz, 3H). LC method A.

### Example 54: Preparation of Compound 54

### Step 1: 2-[4-(4-tert-Butylphenyl)-4-hydroxy-butyl]isoindoline-1,3-dione

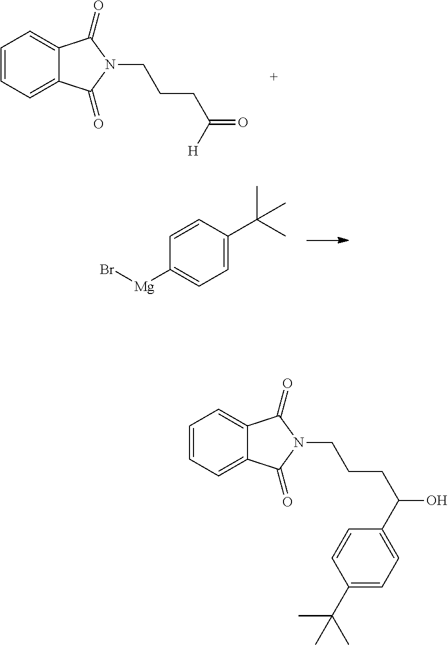

To a solution of 4-(1,3-dioxoisoindolin-2-yl)butanal (933 mg, 4.295 mmol) in THE (17 mL) at −78° C. was added bromo-(4-tert-butylphenyl)magnesium (8.6 mL of 0.5 M, 4.300 mmol). The reaction was warmed to 23° C. and further stirred for 30 min. A saturated aqueous solution of sodium bicarbonate was added and then partitioned with diethyl ether. The organic layer was separated, and the aqueous layer was extracted once more with diethyl ether. The combined organics were washed with brine, dried over magnesium sulfate, filtered and concentrated in vacuo. The crude residue was separated by flash column chromatography on silica gel (10 to 90% ethyl acetate in hexanes) which afforded 2-[4-(4-tert-butylphenyl)-4-hydroxy-butyl]isoindoline-1,3-dione (723 mg, 48%) ¹H NMR (400 MHz, Chloroform-d) δ 7.90-7.79 (m, 2H), 7.75-7.66 (m, 2H), 7.40-7.32 (m, 2H), 7.32-7.23 (m, 2H), 4.70 (dd, J 7.4, 5.0 Hz, 1H), 3.73 (td, J 7.0, 1.8 Hz, 2H), 2.07 (s, 1H), 1.94-1.63 (m, 4H), 1.30 (s, 9H). ESI-MS m/z calc. 351.18344, Retention time: 0.73 minutes; LC method D

### Step 2: 4-Amino-1-(4-tert-butylphenyl)butan-1-ol

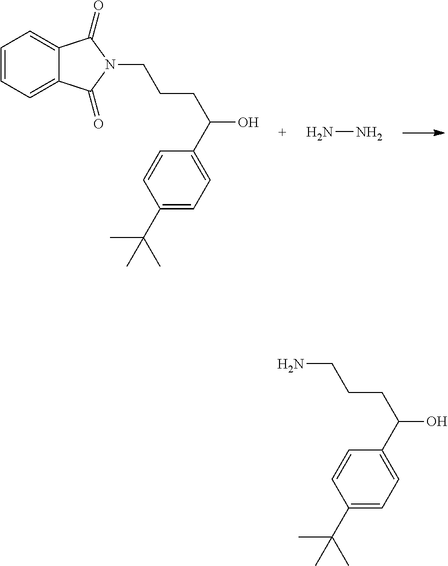

To a solution of 2-[4-(4-tert-butylphenyl)-4-hydroxy-butyl]isoindoline-1,3-dione (723 mg, 2.057 mmol) in methanol was added hydrazine hydrate (808 mg of 65% w/v, 10.28 mmol). The reaction mixture was stirred for 3 h and then the solvent was removed in vacuo. The crude residue was partitioned between potassium hydroxide (11.5 mL of 10% w/v, 20.50 mmol), water and dichloromethane. The organic layer was separated, and the aqueous layer was further extracted with dichloromethane (3×). The combined organics were dried over sodium sulfate, filtered, and concentrated. The intermediate 4-amino-1-(4-tert-butylphenyl)butan-1-ol (440 mg, 85%) ESI-MS m/z calc. 221.17796, found 223.27 (M+1)⁺; Retention time: 0.43 minutes, LC method D.

### Step 3: 10-(4-tert-butylphenyl)-6-(2,6-dimethylphenyl)-2,2-dioxo-9-oxa-2λ⁶-thia-3,5,14,21-tetrazatricyclo[14.3.1.14,8]henicosa-1(20),4(21),5,7,16,18-hexaen-15-one (Compound 54)

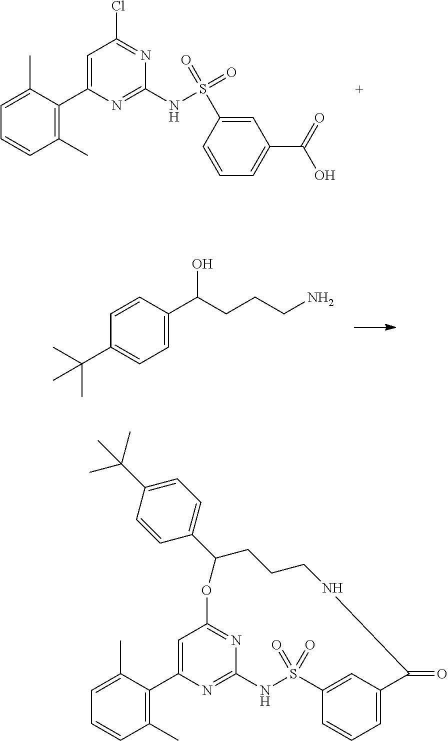

To a solution of 3-[[4-chloro-6-(2,6-dimethylphenyl)pyrimidin-2-yl]sulfamoyl]benzoic acid (146 mg, 0.3494 mmol) and 4-amino-1-(4-tert-butylphenyl)butan-1-ol (hydrochloride salt) (60 mg, 0.2327 mmol) in DMF (1.2 mL) at 0° C. was added potassium tert-butoxide (131 mg, 1.167 mmol). The reaction was allowed to warm to 23° C. and stirred for 1 h. The reaction mixture was cooled to 0° C. and acidified with hydrochloric acid (150 μL of 12 M, 1.800 mmol). The sample was purified by reverse phase HPLC (Waters Sunfire C₁₈ column (100×50 mm, 10 m particle size), gradient: 1-99% acetonitrile in water (5 mM HCl) over 15.0 minutes) which afforded the intermediate 3-[[4-[4-amino-1-(4-tert-butylphenyl)butoxy]-6-(2,6-dimethylphenyl)pyrimidin-2-yl]sulfamoyl]benzoic acid (hydrochloride salt) (70 mg, 47%) ESI-MS m/z calc. 602.2563, found 603.49 (M+1)⁺; Retention time: 0.6 minutes (LC method D).

The intermediate was dissolved in DMF (400 μL) and HATU (125 mg, 0.3287 mmol) was added. The resulting mixture was heated to 50° C. for 10 min and triethylamine (130 μL, 0.9327 mmol) was added. The solution was further stirred for 30 min. The sample was purified by reverse phase HPLC (Phenomenex Luna C₁₈ column (75×30 mm, 5 m particle size), gradient: 1-99% acetonitrile in water (5 mM HCl) over 15.0 minutes) which afforded 10-(4-tert-butylphenyl)-6-(2,6-dimethylphenyl)-2,2-dioxo-9-oxa-2λ⁶-thia-3,5,14,21-tetrazatricyclo[14.3.1.14,8]henicosa-1(20),4(21),5,7,16,18-hexaen-15-one (2.5 mg) ESI-MS m z calc. 584.2457, found 585.48 (M+1)⁺; Retention time: 1.99 minutes; LC method A.

### Example 55: Preparation of Compound 55

### Step 1: 3-[[4-[3-Amino-1-(4-tert-butylphenyl)propoxy]-6-(2,6-dimethylphenyl)pyrimidin-2-yl]sulfamoyl]benzoic Acid

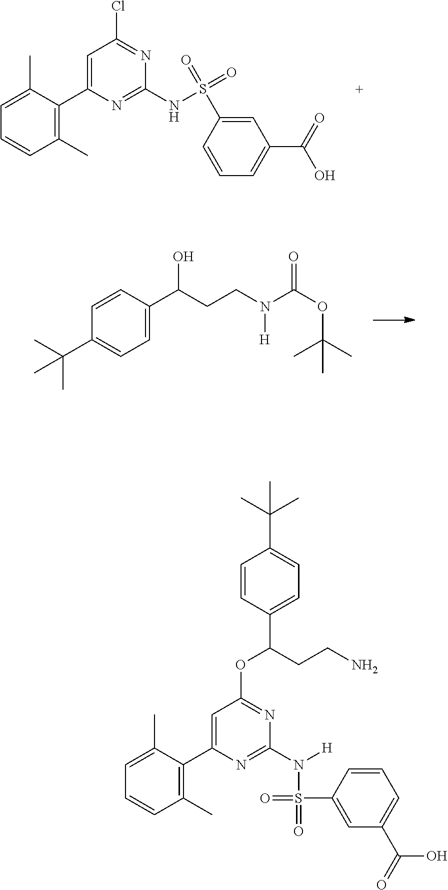 [SMILES: Cc1cccc(C)c1-c1cc(Cl)nc(NS(=O)(=O)c2cccc(C(=O)O)c2)n1 ; CC(C)(C)OC(=O)NCCC(O)c1ccc(C(C)(C)C)cc1 ; Cc1cccc(C)c1-c1cc(OC(CCN)c2ccc(C(C)(C)C)cc2)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (154.7 mg, 0.3702 mmol) and tert-butyl N-[3-(4-tert-butylphenyl)-3-hydroxy-propyl]carbamate (trifluoroacetate salt) (130 mg, 0.3085 mmol) in DMF (1.5 mL) was added potassium tert-butoxide (173 mg, 1.542 mmol) after 10 min of stirring 23° C. the reaction mixture was heated to 70° C. for 2 h. The sample was purified by reverse phase HPLC (Waters Sunfire C₁₈ column (100×50 mm, 10 m particle size), gradient: 1-99% acetonitrile in water (5 mM HCl) over 15.0 minutes) which afforded 3-[[4-[3-amino-1-(4-tert-butylphenyl)propoxy]-6-(2,6-dimethylphenyl)pyrimidin-2-yl]sulfamoyl]benzoic acid (hydrochloride salt) (12 mg, 6%) ESI-MS m/z calc. 588.24066, found 589.23 (M+1)⁺; Retention time: 0.65 minutes; LC method D.

### Step 2: 10-(4-tert-Butylphenyl)-6-(2,6-dimethylphenyl)-2,2-dioxo-9-oxa-2λ⁶-thia-3,5,13,20-tetrazatricyclo[13.3.1.14,8]icosa-1(19),4(20),5,7,15,17-hexaen-14-one (Compound 55)

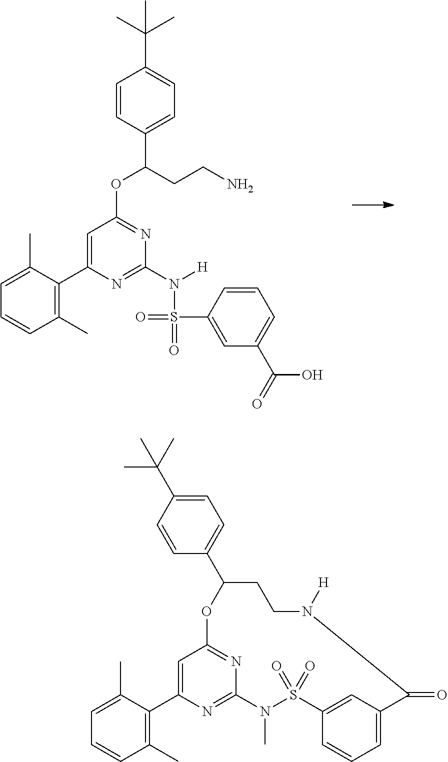 [SMILES: Cc1cccc(C)c1-c1cc(OC(CCN)c2ccc(C(C)(C)C)cc2)nc(NS(=O)(=O)c2cccc(C(=O)O)c2)n1 ; Cc1cccc(C)c1-c1cc2nc(n1)N(C)S(=O)(=O)c1cccc(c1)C(=O)NCCC(c1ccc(C(C)(C)C)cc1)O2]

To a solution of 3-[[4-[3-amino-1-(4-tert-butylphenyl)propoxy]-6-(2,6-dimethylphenyl)pyrimidin-2-yl]sulfamoyl]benzoic acid (hydrochloride salt) (12 mg, 0.02038 mmol) in DMF (100 μL) was added HATU (11.6 mg, 0.03051 mmol). The reaction mixture was heated to 50° C. for 5 min and triethylamine (10 μL, 0.07175 mmol) was added. The reaction was further stirred at this temperature for 10 min. The sample was purified by reverse phase HPLC (Phenomenex Luna C₁₈ column (75×30 mm, 5 m particle size), gradient: 1-99% acetonitrile in water (5 mM HCl) over 15.0 minutes) which afforded 10-(4-tert-butylphenyl)-6-(2,6-dimethylphenyl)-2,2-dioxo-9-oxa-2λ⁶-thia-3,5,13,20-tetrazatricyclo[13.3.1.14,8]icosa-1(19),4(20),5,7,15,17-hexaen-14-one (2.1 mg, 18%) ESI-MS m/z calc. 570.2301, found 571.43 (M+1)⁺; Retention time: 1.93 minutes; LC method A.

### Example 56: Preparation of Compound 56 and Compound 57

### Step 1: 3-[[4-[(3R,4R)-4-Amino-1-tert-butoxycarbonyl-pyrrolidin-3-yl]oxy-6-(2,6-dimethylphenyl)pyrimidin-2-yl]sulfamoyl]benzoic Acid

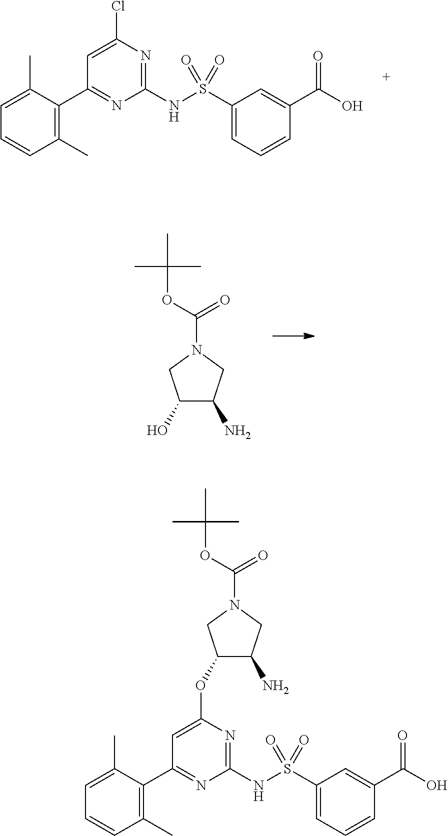 [SMILES: Cc1cccc(C)c1-c1cc(Cl)nc(NS(=O)(=O)c2cccc(C(=O)O)c2)n1 ; CC(C)(C)OC(=O)N1C[C@@H](N)[C@H](O)C1 ; Cc1cccc(C)c1-c1cc(O[C@@H]2CN(C(=O)OC(C)(C)C)C[C@H]2N)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (10.6 g, 25.37 mmol), tert-butyl (3R,4R)-3-amino-4-hydroxy-pyrrolidine-1-carboxylate (5.2 g, 25.71 mmol), and sodium t-butoxide (7.3 g, 75.96 mmol) in THE (0.13 L) was stirred for 18 hours. The reaction was acidified with 1 M citric acid, diluted with water, and extracted with ethyl acetate. The combined extracts were washed with brine, dried over sodium sulfate, and evaporated under vacuum to give a tan oil. The oil was stirred with diethyl ether to give a colorless solid. The solid was filtered, washed with diethyl ether, and dried under vacuum to give 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (15.2 g, 103%) ESI-MS m/z calc. 583.2101, found 584.3 (M+1)⁺; Retention time: 0.49 minutes as a colorless solid. LC method D.

### Step 2: tert-Butyl (3R,7R)-19-(2,6-dimethylphenyl)-9,15,15-trioxo-2-oxa-15λ⁶-thia-5,8,16,18,21-pentaazatetracyclo[15.3.1.110,14.03,7]docosa-1(20),10(22),11,13,17(21),18-hexaene-5-carboxylate (Compound 57)

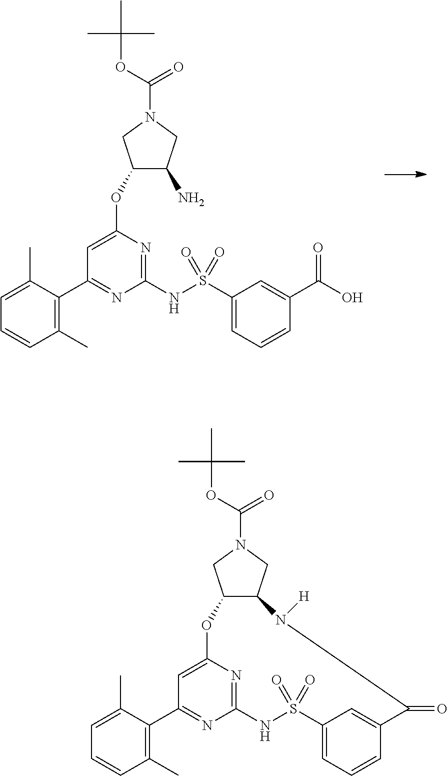 [SMILES: Cc1cccc(C)c1-c1cc(O[C@@H]2CN(C(=O)OC(C)(C)C)C[C@H]2N)nc(NS(=O)(=O)c2cccc(C(=O)O)c2)n1 ; Cc1cccc(C)c1-c1cc2nc(n1)NS(=O)(=O)c1cccc(c1)C(=O)N[C@@H]1CN(C(=O)OC(C)(C)C)C[C@H]1O2]

A solution of 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (1.0 g, 1.713 mmol), HATU (0.98 g, 2.577 mmol), and DIEA (0.9 mL, 5.167 mmol) in DMF (0.1 L) was stirred for three days. The reaction was diluted with water and extracted with ethyl acetate. The combined extracts were washed with brine and water, dried over sodium sulfate, and evaporated under vacuum. The residue was purified by silica gel column chromatography with 0-8% methanol in dichloromethane to give tert-butyl (3R,7R)-19-(2,6-dimethylphenyl)-9,15,15-trioxo-2-oxa-15λ⁶-thia-5,8,16,18,21-pentaazatetracyclo[15.3.1.110,14.03,7]docosa-1(20),10(22),11,13,17(21),18-hexaene-5-carboxylate (0.18 g, 18%) as a light tan solid. A small portion of the product was re-purified by reverse-phase HPLC-MS (1%-99% acetonitrile/water). ESI-MS m/z calc. 565.1995, found 566.3 (M+1)⁺; Retention time: 1.5 minutes, LC method A.

### Step 3: (3R,7R)-19-(2,6-Dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione

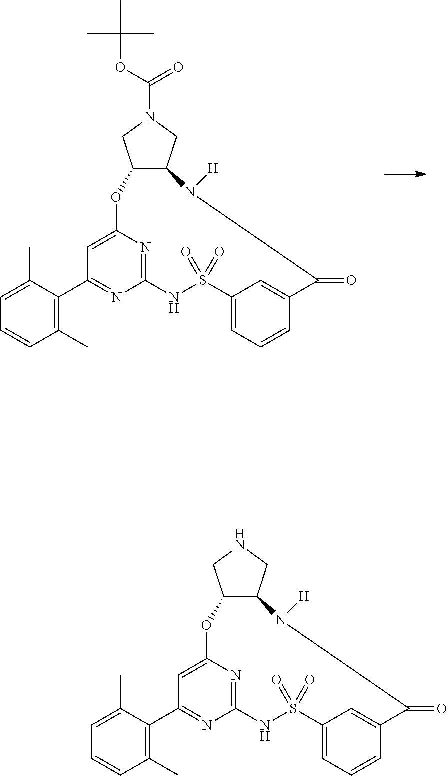 [SMILES: Cc1cccc(C)c1-c1cc2nc(n1)NS(=O)(=O)c1cccc(c1)C(=O)N[C@@H]1CN(C(=O)OC(C)(C)C)C[C@H]1O2 ; Cc1cccc(C)c1-c1cc2nc(n1)NS(=O)(=O)c1cccc(c1)C(=O)N[C@@H]1CNC[C@H]1O2]

A mixture of tert-butyl (3R,7R)-19-(2,6-dimethylphenyl)-9,15,15-trioxo-2-oxa-15λ⁶-thia-5,8,16,18,21-pentaazatetracyclo[15.3.1.110,14.03,7]docosa-1(20),10(22),11,13,17(21),18-hexaene-5-carboxylate (0.16 g, 0.2829 mmol) in HCl (3 mL of 4 M, 12.00 mmol) (in dioxane) was stirred for three hours. The solvent was removed under vacuum, and the solids were triturated with diethyl ether to give (3R,7R)-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (0.14 g, 99%) as a light tan solid. ESI-MS m/z calc. 465.14706, found 466.2 (M+1)⁺; Retention time: 0.28 minutes, LC method D.

### Step 4: (3R,7R)-19-(2,6-Dimethylphenyl)-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 56)

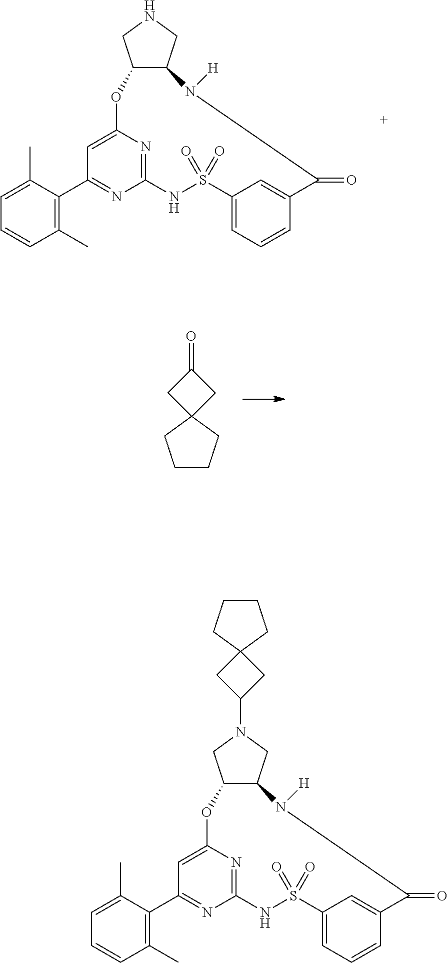 [SMILES: Cc1cccc(C)c1-c1cc2nc(n1)NS(=O)(=O)c1cccc(c1)C(=O)N[C@@H]1CNC[C@H]1O2 ; O=C1CC2(CCCC2)C1 ; Cc1cccc(C)c1-c1cc2nc(n1)NS(=O)(=O)c1cccc(c1)C(=O)N[C@@H]1CN(C3CC4(CCCC4)C3)C[C@H]1O2]

A solution of (3R,7R)-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (30.12 mg, 0.06 mmol), spiro[3.4]octan-2-one (approximately 22.35 mg, 0.1800 mmol), and sodium triacetoxyborohydride (approximately 50.87 mg, 0.2400 mmol) in dichloromethane (300.0 μL) was stirred for two hours. The reaction was stirred with methanol, the volatiles were removed under vacuum, and the residue was purified by reverse-phase HPLC-MS (1%-99% acetonitrile/water (5 mM HCl)) to give (3R,7R)-19-(2,6-dimethylphenyl)-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (23.1 mg, 62%). ESI-MS m/z calc. 573.24097, found 574.3 (M+1)⁺; Retention time: 1.21 minutes; LC method A.

### Example 57: Preparation of Compound 58 and Compound 59

### Step 1: 3-[[4-[[(3R,4R)-3-Amino-1-tert-butoxycarbonyl-4-piperidyl]oxy]-6-(2,6-dimethylphenyl)pyrimidin-2-yl]sulfamoyl]benzoic Acid

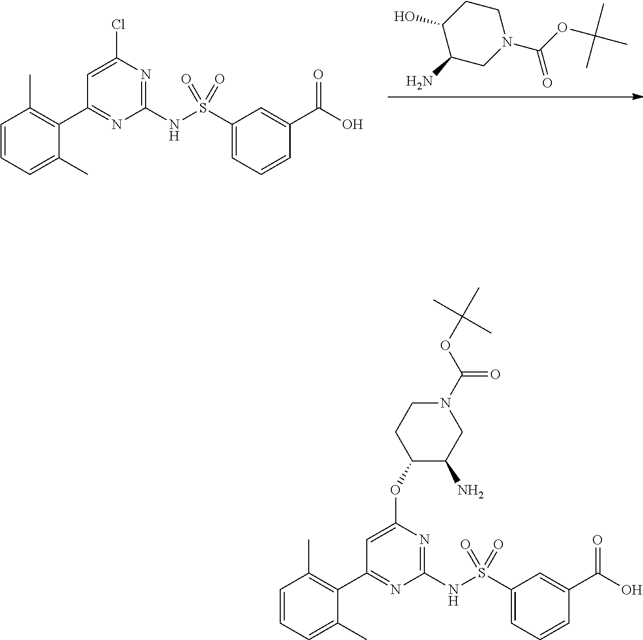 [SMILES: Cc1cccc(C)c1-c1cc(Cl)nc(NS(=O)(=O)c2cccc(C(=O)O)c2)n1 ; CC(C)(C)OC(=O)N1CC[C@@H](O)[C@H](N)C1 ; Cc1cccc(C)c1-c1cc(O[C@@H]2CCN(C(=O)OC(C)(C)C)C[C@H]2N)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (0.63 g, 1.508 mmol), tert-butyl (3R,4R)-3-amino-4-hydroxy-piperidine-1-carboxylate (0.33 g, 1.526 mmol), and sodium t-butoxide (0.44 g, 4.578 mmol) in THE (8 mL) was stirred for two days. The reaction was acidified with 1 M citric acid, diluted with water, and extracted with ethyl acetate. The combined extracts were dried over sodium sulfate and evaporated under vacuum. The residue was purified by silica gel column chromatography with 1-18% methanol in dichloromethane to give a mixture of product and silica gel. The solids were washed with warm ethyl acetate and dichloromethane, and the combined washes were evaporated under vacuum to give 3-[[4-[[(3R,4R)-3-amino-1-tert-butoxycarbonyl-4-piperidyl]oxy]-6-(2,6-dimethylphenyl)pyrimidin-2-yl]sulfamoyl]benzoic acid (0.87 g, 97%) as a light tan solid. ESI-MS m/z calc. 597.2257, found 598.3 (M+1)⁺; Retention time: 0.48 minutes, LC method D.

### Step 2: tert-Butyl (3R,8R)-20-(2,6-dimethylphenyl)-10,16,16-trioxo-2-oxa-16λ⁶-thia-6,9,17,19,22-pentaazatetracyclo[16.3.1.111,15.03,8]tricosa-1(21),11(23),12,14,18(22),19-hexaene-6-carboxylate (Compound 59)

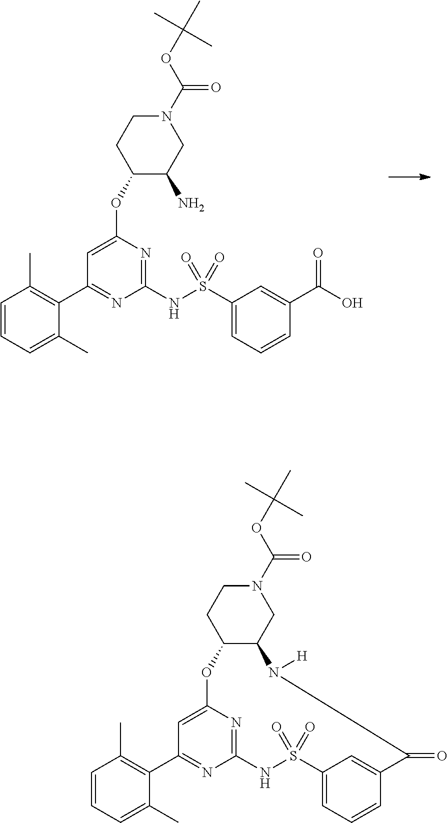 [SMILES: Cc1cccc(C)c1-c1cc(O[C@@H]2CCN(C(=O)OC(C)(C)C)C[C@H]2N)nc(NS(=O)(=O)c2cccc(C(=O)O)c2)n1 ; Cc1cccc(C)c1-c1cc2nc(n1)NS(=O)(=O)c1cccc(c1)C(=O)N[C@@H]1CN(C(=O)OC(C)(C)C)CC[C@H]1O2]

A solution of 3-[[4-[[(3R,4R)-3-amino-1-tert-butoxycarbonyl-4-piperidyl]oxy]-6-(2,6-dimethylphenyl)pyrimidin-2-yl]sulfamoyl]benzoic acid (0.87 g, 1.456 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (0.95 g, 2.223 mmol) (COMU), and DIEA (0.76 mL, 4.363 mmol) in DMF (75 mL) was stirred for 19 hours. The reaction was acidified with 1 M citric acid, diluted with water and extracted with ethyl acetate. The combined extracts were washed with brine, dried over sodium sulfate, and evaporated under vacuum. The residue was purified by silica gel column chromatography with 0-8% methanol in dichloromethane to give tert-butyl (3R,8R)-20-(2,6-dimethylphenyl)-10,16,16-trioxo-2-oxa-16λ⁶-thia-6,9,17,19,22-pentaazatetracyclo[16.3.1.111,15.03,8]tricosa-1(21),11(23),12,14,18(22),19-hexaene-6-carboxylate (0.29 g, 34%) as an off-white solid. ESI-MS m/z calc. 579.21515, found 580.3 (M+1)⁺; Retention time: 1.58 minutes, LC method A.

### Step 3: (3R,8R)-20-(2,6-Dimethylphenyl)-2-oxa-16λ⁶-thia-6,9,17,19,22-pentaazatetracyclo[16.3.1.111,15.03,8]tricosa-1(21),11(23),12,14,18(22),19-hexaene-10,16,16-trione

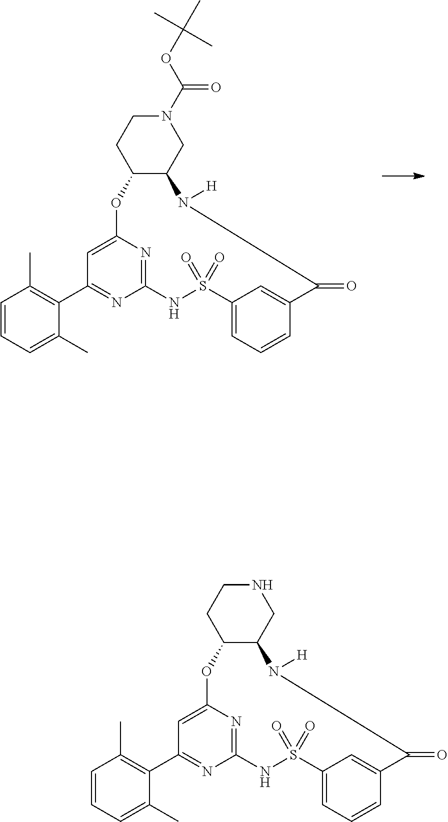 [SMILES: Cc1cccc(C)c1-c1cc2nc(n1)NS(=O)(=O)c1cccc(c1)C(=O)N[C@@H]1CN(C(=O)OC(C)(C)C)CC[C@H]1O2 ; Cc1cccc(C)c1-c1cc2nc(n1)NS(=O)(=O)c1cccc(c1)C(=O)N[C@@H]1CNCC[C@H]1O2]

A solution of tert-butyl (3R,8R)-20-(2,6-dimethylphenyl)-10,16,16-trioxo-2-oxa-16λ⁶-thia-6,9,17,19,22-pentaazatetracyclo[16.3.1.111,15.03,8]tricosa-1(21),11(23),12,14,18(22),19-hexaene-6-carboxylate (0.27 g, 0.4658 mmol) in HCl (5 mL of 4 M, 20.00 mmol) (in dioxane) was stirred for three hours. The solvent was removed under vacuum, and the residue was triturated with diethyl ether and dried under vacuum to give (3R,8R)-20-(2,6-dimethylphenyl)-2-oxa-16λ⁶-thia-6,9,17,19,22-pentaazatetracyclo[16.3.1.111,15.03,8]tricosa-1(21),11(23),12,14,18(22),19-hexaene-10,16,16-trione (hydrochloride salt) (0.24 g, 100%) as a colorless solid. ESI-MS m/z calc. 479.16272, found 480.2 (M+1)⁺; Retention time: 0.29 minutes (LC method D).

### Step 4: (3R,8R)-20-(2,6-Dimethylphenyl)-6-{2-[1-(trifluoromethyl)cyclopropyl]ethyl}-2-oxa-16λ⁶-thia-6,9,17,19,22-pentaazatetracyclo[16.3.1.111,15.03,8]tricosa-1(21),11,13,15(23),18(22),19-hexaene-10,16,16-trione (Compound 58)

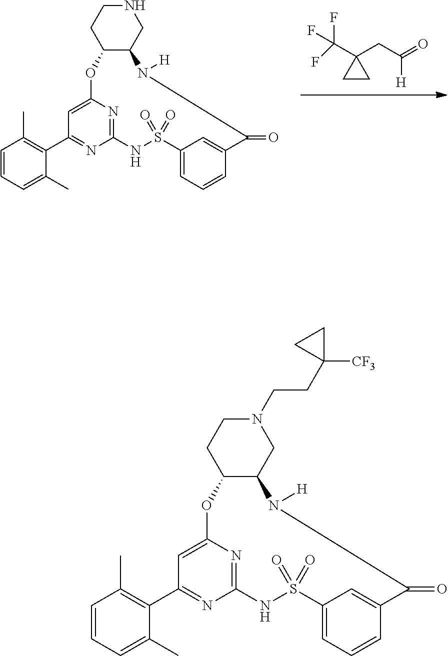 [SMILES: Cc1cccc(C)c1-c1cc2nc(n1)NS(=O)(=O)c1cccc(c1)C(=O)N[C@@H]1CNCC[C@H]1O2 ; O=CCC1(C(F)(F)F)CC1 ; Cc1cccc(C)c1-c1cc2nc(n1)NS(=O)(=O)c1cccc(c1)C(=O)N[C@@H]1CN(CCC3(C(F)(F)F)CC3)CC[C@H]1O2]

A solution of (3R,8R)-20-(2,6-dimethylphenyl)-2-oxa-16λ⁶-thia-6,9,17,19,22-pentaazatetracyclo[16.3.1.111,15.03,8]tricosa-1(21),11(23),12,14,18(22),19-hexaene-10,16,16-trione (hydrochloride salt) (25.80 mg, 0.05 mmol), 2-[1-(trifluoromethyl)cyclopropyl]acetaldehyde (approximately 22.82 mg, 0.1500 mmol), and sodium triacetoxyborohydride (approximately 42.39 mg, 0.2000 mmol) in dichloromethane (0.3 mL) was stirred for 17 hours. The reaction was stirred with methanol, and the solvents were evaporated. The residue was purified by reverse-phase HPLC-MS (1%-99% acetonitrile/water (5 mM HCl)) to give (3R,8R)-20-(2,6-dimethylphenyl)-6-{2-[1-(trifluoromethyl)cyclopropyl]ethyl}-2-oxa-16λ⁶-thia-6,9,17,19,22-pentaazatetracyclo[16.3.1.111,15.03,8]tricosa-1(21),11,13,15(23),18(22),19-hexaene-10,16,16-trione (hydrochloride salt) (21.5 mg, 69%). ESI-MS m/z calc. 615.2127, found 616.3 (M+1)⁺; Retention time: 1.16 minutes; LC method A.

### Example 58: Preparation of Compound 60

### Step 1: (3R,8R)-20-(2,6-Dimethylphenyl)-6-{spiro[3.5]nonan-2-yl}-2-oxa-16λ⁶-thia-6,9,17,19,22-pentaazatetracyclo[16.3.1.111,15.03,8]tricosa-1(21),11,13,15(23),18(22),19-hexaene-10,16,16-trione (Compound 60)

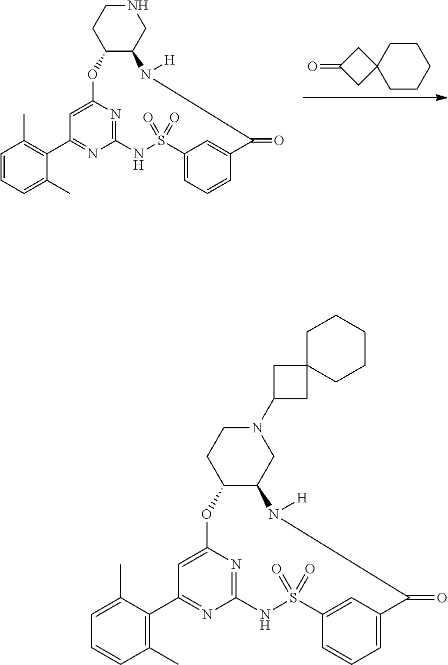 [SMILES: Cc1cccc(C)c1-c1cc2nc(n1)NS(=O)(=O)c1cccc(c1)C(=O)N[C@@H]1CNCC[C@H]1O2 ; O=C1CC2(CCCCC2)C1 ; Cc1cccc(C)c1-c1cc2nc(n1)NS(=O)(=O)c1cccc(c1)C(=O)N[C@@H]1CN(C3CC4(CCCCC4)C3)CC[C@H]1O2]

A solution of (3R,8R)-20-(2,6-dimethylphenyl)-2-oxa-16λ⁶-thia-6,9,17,19,22-pentaazatetracyclo[16.3.1.111,15.03,8]tricosa-1(21),11(23),12,14,18(22),19-hexaene-10,16,16-trione (hydrochloride salt) (25.80 mg, 0.05 mmol), spiro[3.5]nonan-2-one (approximately 20.73 mg, 0.1500 mmol), and sodium triacetoxyborohydride (approximately 42.39 mg, 0.2000 mmol) in dichloromethane (0.3 mL) was stirred for 17 hours. The reaction was stirred with methanol, and the solvents were evaporated. The residue was purified by reverse-phase HPLC-MS (1%-99% acetonitrile/water (5 mM HCl)) to give (3R,8R)-20-(2,6-dimethylphenyl)-6-{spiro[3.5]nonan-2-yl}-2-oxa-16λ⁶-thia-6,9,17,19,22-pentaazatetracyclo[16.3.1.111,15.03,8]tricosa-1(21),11,13,15(23),18(22),19-hexaene-10,16,16-trione (hydrochloride salt) (22.2 mg, 73%). ESI-MS m/z calc. 601.2723, found 602.3 (M+1)⁺; Retention time: 1.26 minutes; LC method A.

### Example 59: Preparation of Compound 61

### Step 1: (3R,8R)-6-Benzyl-20-(2,6-dimethylphenyl)-2-oxa-16λ⁶-thia-6,9,17,19,22-pentaazatetracyclo[16.3.1.111,15.03,8]tricosa-1(21),11,13,15(23),18(22),19-hexaene-10,16,16-trione (Compound 61)

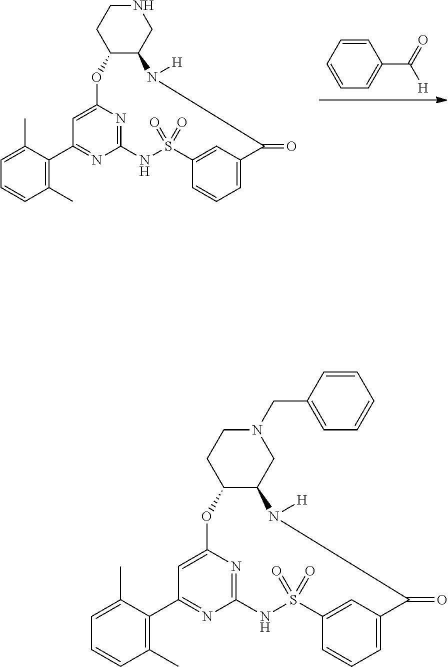 [SMILES: Cc1cccc(C)c1-c1cc2nc(n1)NS(=O)(=O)c1cccc(c1)C(=O)N[C@@H]1CNCC[C@H]1O2 ; O=Cc1ccccc1 ; Cc1cccc(C)c1-c1cc2nc(n1)NS(=O)(=O)c1cccc(c1)C(=O)N[C@@H]1CN(Cc3ccccc3)CC[C@H]1O2]

A solution of (3R,8R)-20-(2,6-dimethylphenyl)-2-oxa-16λ⁶-thia-6,9,17,19,22-pentaazatetracyclo[16.3.1.111,15.03,8]tricosa-1(21),11(23),12,14,18(22),19-hexaene-10,16,16-trione (hydrochloride salt) (25.80 mg, 0.05 mmol), benzaldehyde (approximately 15.92 mg, 15.25 μL, 0.1500 mmol), and sodium triacetoxyborohydride (approximately 42.39 mg, 0.2000 mmol) in dichloromethane (0.3 mL) was stirred for 17 hours. The reaction was stirred with methanol, and the solvents were evaporated. The residue was purified by reverse-phase HPLC-MS (1%-99% acetonitrile/water (5 mM HCl)) to give (3R,8R)-6-benzyl-20-(2,6-dimethylphenyl)-2-oxa-16λ⁶-thia-6,9,17,19,22-pentaazatetracyclo[16.3.1.111,15.03,8]tricosa-1(21),11,13,15(23),18(22),19-hexaene-10,16,16-trione (hydrochloride salt) (20.5 mg, 68%). ESI-MS m/z calc. 569.20966, found 570.2 (M+1)⁺; Retention time: 1.05 minutes; LC method A.

### Example 60: Preparation of Compound 62

### Step 1: (3R,7R)-19-(2,6-Dimethylphenyl)-5-[5-(trifluoromethyl)pyridin-2-yl]-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 62)

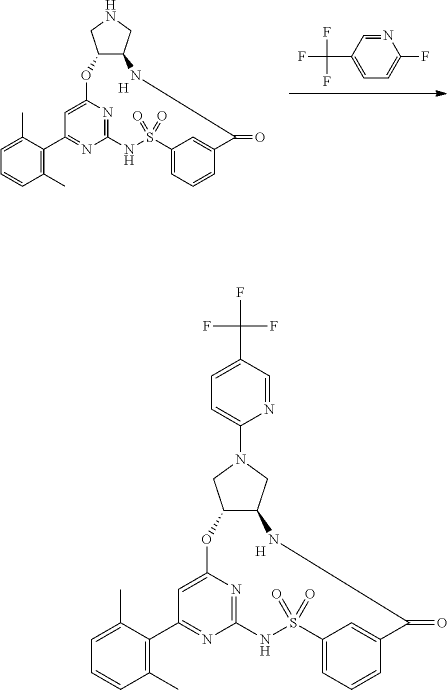

A solution of (3R,7R)-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (16 mg, 0.03187 mmol), 2-fluoro-5-(trifluoromethyl)pyridine (8 mg, 0.04846 mmol), and DIEA (18 μL, 0.1033 mmol) in dioxane (0.2 mL) was stirred for four hours. The reaction was stirred at 60° C. for two hours then at room temperature for three days and then at 60° C. for five hours. The solvent was evaporated, and the residue was purified by reverse-phase HPLC-MS (1%-99% acetonitrile/water (5 mM HCl)) to give (3R,7R)-19-(2,6-dimethylphenyl)-5-[5-(trifluoromethyl)pyridin-2-yl]-2-oxa-15λ⁶-thia-5,8,16,18,21-pentaazatetracyclo[15.3.1.110,14.03,7]docosa-1(20),10(22),11,13,17(21),18-hexaene-9,15,15-trione (6.8 mg, 35%) ESI-MS m/z calc. 610.161, found 611.3 (M+1)⁺; Retention time: 1.53 minutes, LC method A.

### Example 61: Preparation of Compound 63

### Step 1: (3R,7R)-5-[(4-tert-Butylphenyl)methyl]-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 63)

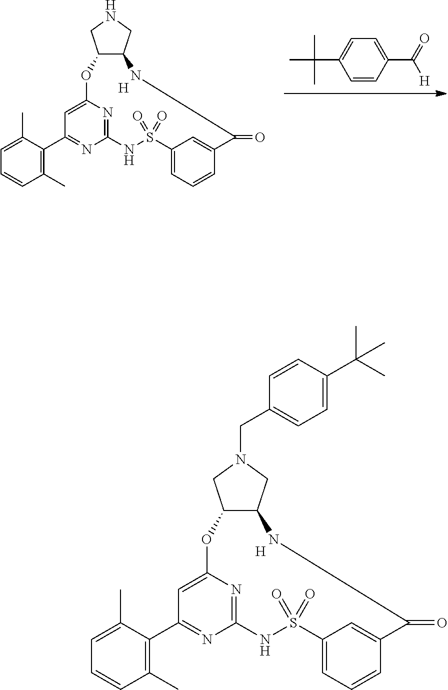

A solution of (3R,7R)-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (25.10 mg, 0.05 mmol), 4-tert-butylbenzaldehyde (approximately 24.33 mg, 25.11 μL, 0.1500 mmol), and sodium triacetoxyborohydride (approximately 42.39 mg, 0.2000 mmol) in dichloromethane (500.0 μL) was stirred for one hour. The reaction was stirred with methanol, the volatiles were removed under vacuum, and the residue was purified by reverse-phase HPLC-MS (20%-80% acetonitrile/water (5 mM HCl)) to give (3R,7R)-5-[(4-tert-butylphenyl)methyl]-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (16.4 mg, 50%) as a colorless solid. ESI-MS m/z calc. 611.25665, found 612.3 (M+1)⁺; Retention time: 1.35 minutes; LC method A.

### Example 62: Preparation of Compound 64

### Step 1: (3R,7R)-5-(3,3-Dimethylbutyl)-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 64)

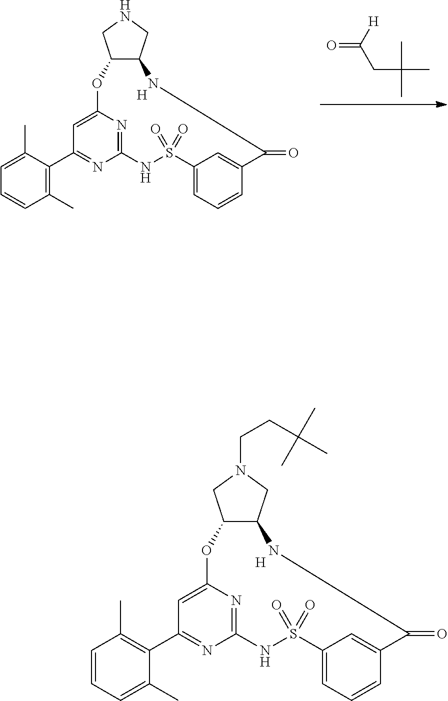

A solution of (3R,7R)-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (30.12 mg, 0.06 mmol), 3,3-dimethylbutanal (approximately 18.03 mg, 22.59 μL, 0.1800 mmol), and sodium triacetoxyborohydride (approximately 50.87 mg, 0.2400 mmol) in dichloromethane (300.0 μL) was stirred for two hours. The reaction was stirred with methanol, the volatiles were removed under vacuum, and the residue was purified by reverse-phase HPLC-MS (1%-99% acetonitrile/water (5 mM HCl)) to give (3R,7R)-5-(3,3-dimethylbutyl)-19-(2,6-dimethylphenyl)-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (21.1 mg, 59%). ESI-MS m/z calc. 549.24097, found 550.3 (M+1)⁺; Retention time: 1.1 minutes; LC method A.

### Example 63: Preparation of Compound 65

### Step 1: Benzyl 2-[(4R)-2-oxooxazolidin-4-yl]acetate

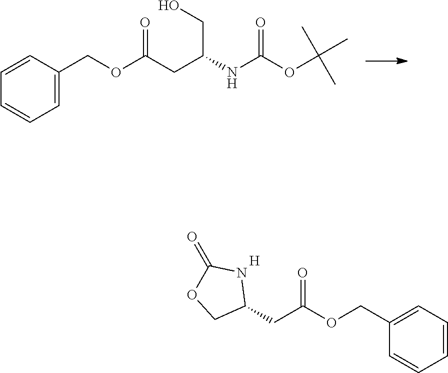

To a solution of benzyl (3R)-3-(tert-butoxycarbonylamino)-4-hydroxy-butanoate (27.8 g, 89.864 mmol)benzyl (3R)-3-(tert-butoxycarbonylamino)-4-hydroxy-butanoate (27.8 g, 89.864 mmol) in 1,2-dichloroethane (250 mL) was added pyridine (65.526 g, 67 mL, 828.40 mmol) and the mixture was cooled to 0-5° C. p-toluenesulfonic anhydride (32.263 g, 98.850 mmol) was added and the mixture was warmed to room temperature and stirred for 2 hours and then heated to 90° C. for 2 hours. The mixture was cooled, diluted with dichloromethane (500 mL) and washed with 1N HCl (3×200 mL). The combined aqueous layers were backextracted with dichloromethane (2×150 mL). The combined organic layers were dried with sodium sulfate, filtered and concentrated to dryness. The crude material was purified by flash chromatography (330 g) using a gradient of 20% to 100% ethyl acetate in heptane to afford enantiopure benzyl 2-[(4R)-2-oxooxazolidin-4-yl]acetate (18.11 g, 86%) as a white solid. ¹H NMR (400 MHz, CDCl₃) δ 7.44-7.31 (m, 5H), 5.58 (br. s., 1H), 5.16 (s, 2H), 4.56 (t, J 8.6 Hz, 1H), 4.25 (qd, J 7.0, 5.9 Hz, 1H), 4.06 (dd, J 8.9, 5.7 Hz, 1H), 2.76-2.63 (m, 2H). ESI-MS m/z calc. 235.0845, found 236.2 (M+1)⁺, 471.2 (2M+H)⁺; Retention time: 1.49 minutes; LC method X.

### Step 2: (4R)-4-(2-Hydroxy-2-methyl-propyl)oxazolidin-2-one

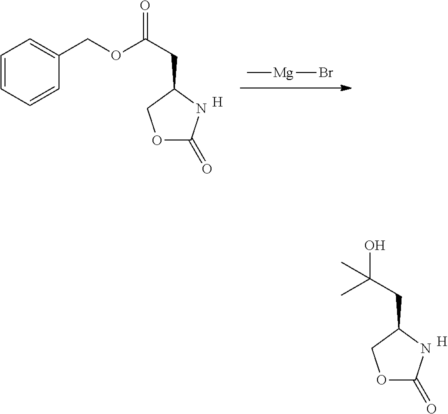

Bromo(methyl)magnesium in diethyl ether (105 mL of 3 M, 315.00 mmol) was added to a mixture of toluene (150 mL) and THE (150 mL) at −20° C. A warm THE (80 mL) solution of benzyl 2-[(4R)-2-oxooxazolidin-4-yl]acetate (18.1 g, 76.944 mmol) was then added dropwise maintaining the temperature below −10° C. The mixture was warm up to room temperature and stirred for 18 hours. The mixture was added via canula to a solution of acetic acid (85 mL) in water (440 mL) at 0° C. The resultant mixture was stirred for 1 hour at room temperature. The layers were separated. The aqueous layer was saturated with brine (200 mL) and further extracted with 2-methyltetrahydrofuran (3×250 mL) and with ethanol/chloroform (½, 3×330 mL). The combined organic extracts were dried over anhydrous sodium sulafte, filtered and concentrated. The residue was co-evaporated with heptanes (4×100 mL). The crude material was purified in two equal batches by flash chromatography (330 g) eluting with 6% isopropanol in dichloromethane) to give (4R)-4-(2-hydroxy-2-methyl-propyl)oxazolidin-2-one (8.88 g, 69%) as an off-white solid. ¹H NMR (400 MHz, DMSO-d₆) δ 7.36 (s, 1H), 4.45-4.38 (m, 1H), 4.36 (s, 1H), 4.00-3.91 (m, 2H), 1.68-1.54 (m, 2H), 1.10 (s, 6H). ESI-MS m/z calc. 159.0895, found 160.2 (M+1)⁺; Retention time: 0.77 minutes, LC method X.

### Step 3: (2R)-2-Amino-4-methyl-pentane-1,4-diol

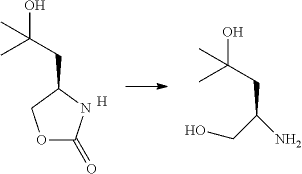

A mixture of (4R)-4-(2-hydroxy-2-methyl-propyl)oxazolidin-2-one (904 mg, 4.2592 mmol) and barium hydroxide octahydrate (4.03 g, 12.775 mmol) in ethanol (20 mL) and water (20 mL) was stirred at 90-95° C. for 4 hours. After cooling down to room temperature, dry ice (˜7 g) was added and the mixture was stirred vigorously for 2 days. The suspension was filtered over a Celite pad and rinsed with ethanol (20 mL). The filtrate was diluted with toluene and concentrated under reduced pressure to provide (2R)-2-amino-4-methyl-pentane-1,4-diol (780 mg) which was used without further purification for the next step. ¹H NMR (400 MHz, DMSO-d₆) δ 5.12 (br. s., 2H), 3.30-3.16 (m, 2H), 2.94 (dd, J 9.0, 3.4 Hz, 1H), 1.83 (s, 2H), 1.49-1.40 (m, 1H), 1.33-1.21 (m, 1H), 1.11 (d, J 11.0 Hz, 6H). ESI-MS m/z calc. 133.1103, found 134.4 (M+1)⁺; Retention time: 0.21 minutes, LC method X.

### Step 4: 3-[[4-[(2R)-2-Amino-4-hydroxy-4-methyl-pentoxy]-6-(2,6-dimethylphenyl)pyrimidin-2-yl]sulfamoyl]benzoic Acid

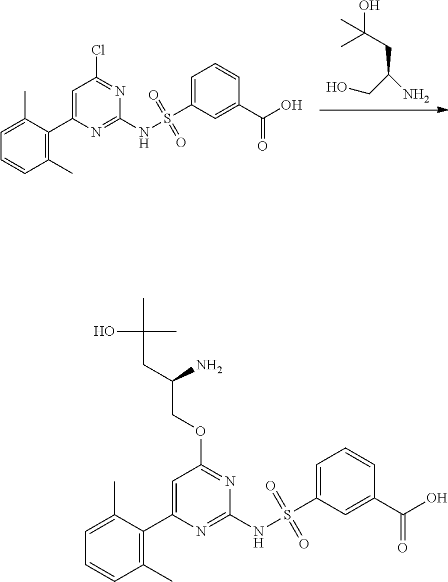

To a solution of (2R)-2-amino-4-methyl-pentane-1,4-diol (567 mg, 4.2571 mmol) and 3-[[4-chloro-6-(2,6-dimethylphenyl)pyrimidin-2-yl]sulfamoyl]benzoic acid (1.5 g, 3.5897 mmol) in tetrahydrofuran (6 mL) was slowly added sodium tert-butoxide in tetrahydrofuran (7.2 mL of 2 M, 14.400 mmol) and the mixture was stirred at room temperature for one hour. The reaction was partitioned between ethyl acetate (30 mL) and 1 N hydrochloric acid (30 mL). The aqueous phase was extracted with ethyl acetate (2×20 mL) and 2-methyltetrahydrofuran (4×30 mL). The combined organic layers were dried over sodium sulfate, filtered and concentrated to dryness. The residue was triturated with ethyl acetate (20 mL), the precipitate was filtered and washed with ethyl acetate (2×10 mL). The product was further dried under vacuum to afford 3-[[4-[(2R)-2-amino-4-hydroxy-4-methyl-pentoxy]-6-(2,6-dimethylphenyl)pyrimidin-2-yl]sulfamoyl]benzoic acid (hydrochloride salt) (1.62 g, 80%) as a pale-yellow solid. ¹H NMR (400 MHz, DMSO-d₆) δ 13.07 (br. s., 2H), 8.43 (s, 1H), 8.14 (d, J 7.8 Hz, 2H), 8.10-8.01 (m, 3H), 7.70 (t, J 7.7 Hz, 1H), 7.32-7.22 (m, 1H), 7.13 (d, J 7.6 Hz, 2H), 6.29 (br. s., 1H), 5.13 (br. s., 1H), 4.36 (dd, J 11.5, 2.9 Hz, 1H), 4.18 (dd, J 11.4, 7.7 Hz, 1H), 3.83-3.70 (m, 1H), 2.02 (s, 6H), 1.71 (d, J 6.4 Hz, 2H), 1.24 (m, 6H). ESI-MS m/z calc. 514.1886, found 515.2 (M+1)⁺; Retention time: 1.3 minutes, LC method X.

### Step 5: (11R)-6-(2,6-Dimethylphenyl)-11-(2-hydroxy-2-methyl-propyl)-2,2-dioxo-9-oxa-2)⁶-thia-3,5,12,19-tetrazatricyclo[12.3.1.14,8]nonadeca-1(18),4(19),5,7,14,16-hexaen-13-one-(Compound-65)

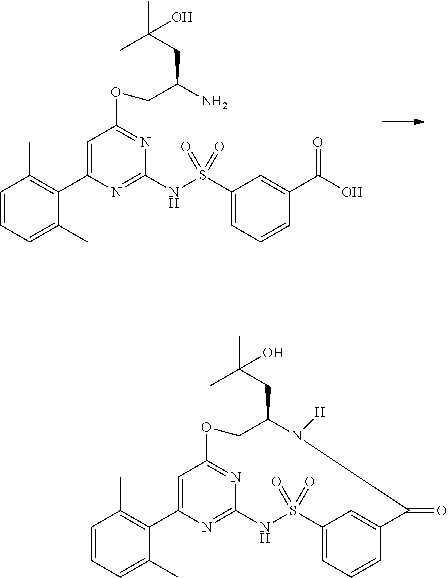 [SMILES: Cc1cccc(C)c1-c1cc(OC[C@H](N)CC(C)(C)O)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CC(C)(C)O)CO2]

To a solution of 3-[[4-[(2R)-2-amino-4-hydroxy-4-methyl-pentoxy]-6-(2,6-dimethylphenyl)pyrimidin-2-yl]sulfamoyl]benzoic acid (hydrochloride salt) (50 mg, 0.0889 mmol) and triethylamine (58.080 mg, 0.08 mL, 0.5740 mmol) in ethyl acetate (3.5 mL) and DMF (1 mL) was added propylphosphonic anhydride solution in ethyl acetate (93 mg, 0.0870 mL, 0.1461 mmol) at 0° C. The ice bath was removed, and the mixture was stirred at room temperature for 2 hours. Propylphosphonic anhydride solution in ethyl acetate (93 mg, 0.0870 mL, 0.1461 mmol) was added and the mixture was left to stir for an additional 2 hours. Ethyl acetate was removed under vacuo and the resulting mixture was purified by reverse phase chromatography (C₁₈ 24 g) using a gradient of 5% to 100% methanol in water to afford (11R)-6-(2,6-dimethylphenyl)-11-(2-hydroxy-2-methyl-propyl)-2,2-dioxo-9-oxa-2,6-thia-3,5,12,19-tetrazatricyclo[12.3.1.14,8]nonadeca-1(18),4(19),5,7,14,16-hexaen-13-one (34.2 mg, 74%) as white powder after lyophilization. ¹H NMR (400 MHz, DMSO-d₆) δ 13.02 (br. s., 1H), 8.51 (s, 1H), 7.92 (d, J 5.9 Hz, 1H), 7.76 (d, J 9.8 Hz, 1H), 7.71-7.57 (m, 2H), 7.25 (t, J 7.8 Hz, 1H), 7.12 (d, J 7.1 Hz, 2H), 6.35 (br. s., 1H), 5.09 (d, J 7.1 Hz, 1H), 4.05 (s, 1H), 3.84 (t, J 11.1 Hz, 1H), 3.53-3.39 (m, 1H), 2.25-1.86 (br. s., 6H), 1.72-1.56 (m, 2H), 0.89-0.70 (m, 6H). ESI-MS m/z calc. 496.178, found 497.1 (M+1)⁺; Retention time: 3.05 minutes, LC method Y.

### Example 64: Preparation of Compound 66

### Step 1: tert-Butyl (4S)-2-(hydroxymethyl)-4-phenyl-oxazolidine-3-carboxylate

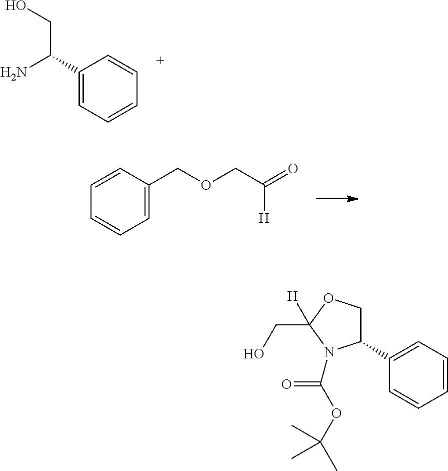 [SMILES: N[C@H](CO)c1ccccc1 ; O=CCOCc1ccccc1 ; CC(C)(C)OC(=O)N1C(CO)OC[C@@H]1c1ccccc1]

In a 100-mL round-bottomed flask, (2S)-2-amino-2-phenyl-ethanol (1.7925 g, 12.81 mmol) was dissolved in dry DCM (40 mL), to which 2-benzyloxyacetaldehyde (1.80 mL, 12.81 mmol) and anhydrous sodium sulfate (3.31 g, 23.30 mmol) were added. This mixture was stirred vigorously at room temperature for 25 h. After this time, TEA (5.0 mL, 35.87 mmol) and Boc anhydride (3.31 g, 15.17 mmol) were added, followed by DMAP (10.5 mg, 0.08595 mmol). This mixture was stirred at room temperature for 2 h, after which a second portion of Boc anhydride (3.31 g, 15.17 mmol) was added and stirred for an additional 13 h. Thereafter, it was filtered over a fritted funnel and evaporated in vacuo to give a yellow liquid. This crude product was purified by silica gel chromatography (120 g of silica, 0 to 30% gradient of ethyl acetate/hexanes) to give tert-butyl (4S)-2-(benzyloxymethyl)-4-phenyl-oxazolidine-3-carboxylate (2.2503 g, 30%) ESI-MS m/z calc. 369.194, found 370.3 (M+1)⁺; Retention time: 2.05 minutes, LC method A.

In a 100-mL round-bottomed flask, the impure product was dissolved in EtOH (40 mL). This solution was sparged with a balloon of hydrogen gas for 5 min. The cap was briefly removed, and Pd(OH)₂/C (1.256 g of 10% w/w, 0.8944 mmol) was added. This reaction mixture was stirred under hydrogen (2 L, 79.37 mmol) at room temperature for 103 h, after which it was filtered through Celite and rinsed with methanol (80 mL). This solution was evaporated in vacuo to give a viscous oil, tert-butyl (4S)-2-(hydroxymethyl)-4-phenyl-oxazolidine-3-carboxylate (1.1509 g, 26%) ESI-MS m/z calc. 279.14706, found 280.2 (M+1)⁺; Retention time: 1.39 minutes, LC method A.

### Step 2: 3-[[4-[[(4S)-3-tert-Butoxycarbonyl-4-phenyl-oxazolidin-2-yl]methoxy]-6-(2,6-dimethylphenyl)pyrimidin-2-yl]sulfamoyl]benzoic Acid

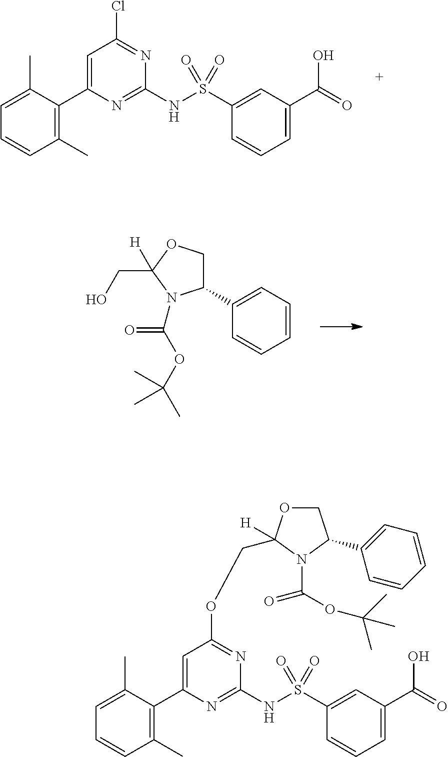 [SMILES: Cc1cccc(C)c1-c1cc(Cl)nc(NS(=O)(=O)c2cccc(C(=O)O)c2)n1 ; CC(C)(C)OC(=O)N1C(CO)OC[C@@H]1c1ccccc1 ; Cc1cccc(C)c1-c1cc(OCC2OC[C@H](c3ccccc3)N2C(=O)OC(C)(C)C)nc(NS(=O)(=O)c2cccc(C(=O)O)c2)n1]

In a 100-mL round-bottomed flask, 3-[[4-chloro-6-(2,6-dimethylphenyl)pyrimidin-2-yl]sulfamoyl]benzoic acid (2.3023 g, 5.510 mmol) and tert-butyl (4S)-2-(hydroxymethyl)-4-phenyl-oxazolidine-3-carboxylate (1.1509 g, 3.708 mmol) were dissolved in NMP (20 mL), and this solution was cooled to 0° C. NaH (0.9031 g of 60% w/w, 22.58 mmol) was added in one portion (CAUTION: evolution of gas and heat), and this mixture was stirred at 0° C. for 5 min then at 50° C. for 15 min. It was then quenched by pouring onto 1 N HCl solution (25 mL), then was extracted with ethyl acetate (3×50 mL). The combined organic extracts were washed with water (100 mL) and saturated aqueous sodium chloride solution (100 mL), then dried over sodium sulfate, filtered, and evaporated in vacuo to give 3 g of a yellow oil. This crude product was purified by silica gel chromatography (120 g of silica, 0 to 80% gradient of ethyl acetate/hexanes) to give a white foam, 3-[[4-[[(4S)-3-tert-butoxycarbonyl-4-phenyl-oxazolidin-2-yl]methoxy]-6-(2,6-dimethylphenyl)pyrimidin-2-yl]sulfamoyl]benzoic acid (4.9812 g, 77%) ESI-MS m/z calc. 660.2254, found 661.4 (M+1)⁺; Retention time: 1.88 minutes, LC method A.

### Step 3: 3-[[4-(2,6-Dimethylphenyl)-6-(2-oxoethoxy)pyrimidin-2-yl]sulfamoyl]benzoic Acid

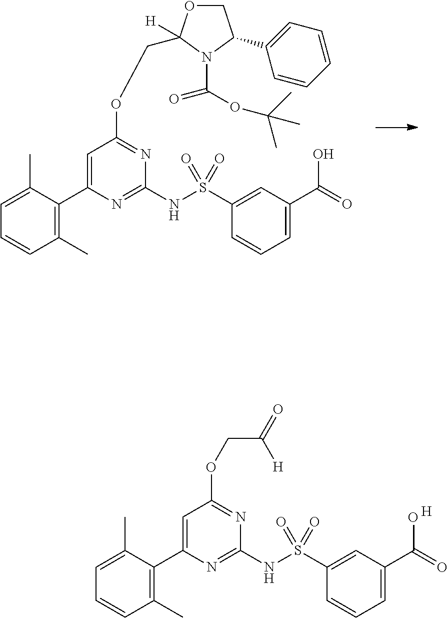

In a 50-mL round-bottomed flask, 3-[[4-[[(4S)-3-tert-butoxycarbonyl-4-phenyl-oxazolidin-2-yl]methoxy]-6-(2,6-dimethylphenyl)pyrimidin-2-yl]sulfamoyl]benzoic acid (4.9812 g, 2.865 mmol) was dissolved in dioxane (12.0 mL), to which a dioxane solution of HCl (4.0 mL of 4.0 M, 16.00 mmol) was added. This solution was stirred at 70° C. for 30 min, after which it was cooled to room temperature and evaporated to dryness in vacuo. This crude product was purified by silica gel chromatography (120 g of silica, 0 to 100% gradient of ethyl acetate/hexanes) to give a white foam, 3-[[4-(2,6-dimethylphenyl)-6-(2-oxoethoxy)pyrimidin-2-yl]sulfamoyl]benzoic acid (0.9135 g, 72%) ESI-MS m/z calc. 441.09946, found 442.3 (M+1)⁺; Retention time: 1.08 minutes. Note: An (M+water+H)⁺ mass of 460.3 is more prominent, LC method A.

### Step 4: N-tert-butyl-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaene-11-carboxamide (Compound 66)

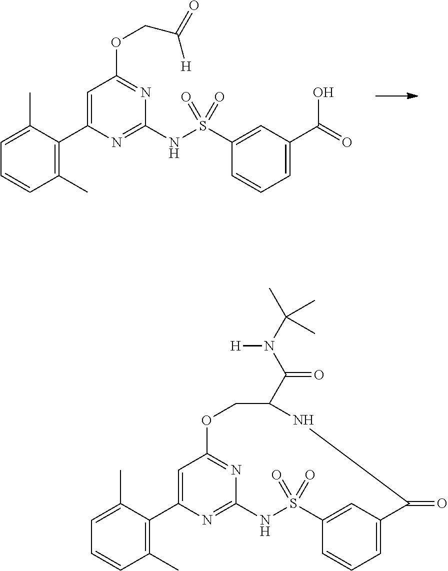 [SMILES: Cc1cccc(C)c1-c1cc(OCC=O)nc(NS(=O)(=O)c2cccc(C(=O)O)c2)n1 ; Cc1cccc(C)c1-c1cc2nc(n1)NS(=O)(=O)c1cccc(c1)C(=O)NC(C(=O)NC(C)(C)C)CO2]

In a 3-mL vial, 3-[[4-(2,6-dimethylphenyl)-6-(2-oxoethoxy)pyrimidin-2-yl]sulfamoyl]benzoic acid (106 mg, 0.2401 mmol) was dissolved in MeOH (1.0 mL), to which 2,4-dimethoxybenzyl amine (41 mg, 0.2452 mmol) and t-butyl isocyanide (20 mg, 0.2406 mmol) were added. This mixture was stirred at room temperature for 1 h, after which it was diluted with MeOH (1 mL), filtered, and purified by reverse phase preparative chromatography using a C₁₈ column and a gradient eluent of 1 to 99% acetonitrile in water containing 5 mM hydrochloric acid to give N-tert-butyl-12-[(2,4-dimethoxyphenyl)methyl]-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaene-11-carboxamide (11.8 mg, 7%) ESI-MS m/z calc. 673.257, found 674.5 (M+1)⁺; Retention time: 2.0 minutes, LC method A.

In a 3-mL vial, the product from above was dissolved in TFA (500 μL, 6.490 mmol), and heated to 50° C. for 30 min. The reaction mixture was then cooled to room temperature, diluted with MeOH (500 μL), filtered, and purified by reverse phase preparative chromatography using a C₁₈ column and a gradient eluent of 1 to 70% acetonitrile in water containing 5 mM hydrochloric acid to give N-tert-butyl-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaene-11-carboxamide (3.7 mg, 3%)¹H NMR (400 MHz, dimethylsulfoxide-d₆) δ 13.37-12.27 (bs, 1H, D₂O exchangeable), 8.61 (s, 1H), 8.06-7.93 (m, 1H), 7.87-7.65 (m, 2H), 7.63 (s, 1H, D₂O exchangeable), 7.41-7.29 (m, 1H, D₂O exchangeable), 7.27 (t, J 7.6 Hz, 1H), 7.14 (d, J 7.6 Hz, 2H), 6.33 (s, 1H), 5.25-5.06 (m, 1H), 4.25-4.09 (m, 2H), 2.12 (s, 6H), 1.16 (s, 9H). ESI-MS m/z calc. 523.18896, found 524.4 (M+1)⁺; Retention time: 1.85 minutes, LC method A.

### Example 65: Preparation of Compound 67

### Step 1: Benzyl (4R)-4-(tert-butoxycarbonylamino)-5-hydroxy-pentanoate

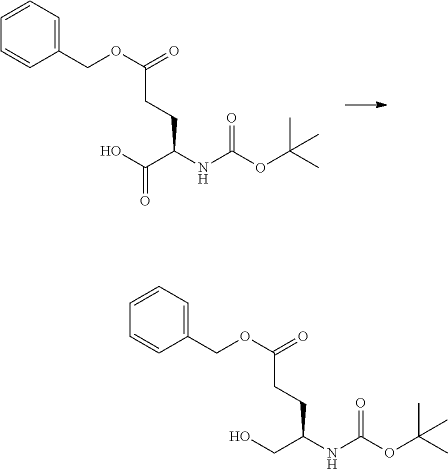 [SMILES: CC(C)(C)OC(=O)N[C@H](CCC(=O)OCc1ccccc1)C(=O)O ; CC(C)(C)OC(=O)N[C@@H](CO)CCC(=O)OCc1ccccc1]

(2R)-5-Benzyloxy-2-(tert-butoxycarbonylamino)-5-oxo-pentanoic acid (10 g, 29.641 mmol) was dissolved in dimethoxyethane (30 mL) and the solution was cooled to −15° C. N-methylmorpholine (3.0360 g, 3.3 mL, 30.016 mmol) was added followed by a slow addition of isobutyl chloroformate (4.1067 g, 3.9 mL, 30.069 mmol) such that the reaction temperature was kept below −10° C. The mixture was stirred for 30 minutes. The solids were quickly filtered and washed with dimethoxyethane (30 mL). The filtrate was cooled to −40° C. and a solution of sodium borohydride (1.45 g, 38.327 mmol) in water (15 mL) was added slowly such that the reaction temperature was maintained between −30° C. and −15° C. The mixture was stirred for 15 minutes. Water (180 mL) was then added dropwise at −15° C. and the temperature was slowly raised to 5° C. while controlling the gas evolution. The suspension was filtered and washed with water (300 mL). The solid was dissolved in dichloromethane (100 mL) and transferred in a separatory funnel. Phases were separated, the organic phase was dried over sodium sulfate, filtered and evaporated to dryness to give benzyl (4R)-4-(tert-butoxycarbonylamino)-5-hydroxy-pentanoate (7.98 g, 83%) as a white solid. ¹H NMR (400 MHz, CDCl₃) δ 7.42-7.30 (m, 5H), 5.13 (s, 2H), 4.81 (br. s., 1H), 3.65 (br. s., 2H), 3.60-3.51 (m, 1H), 2.57-2.36 (m, 3H), 1.98-1.87 (m, 1H), 1.86-1.73 (m, 1H), 1.44 (s, 9H). ESI-MS m/z calc. 323.1733, found 224.4 (M−99)+; Retention time: 1.696 minutes, LC method X.

### Step 2: Benzyl 3-[(4R)-2-oxooxazolidin-4-yl]propanoate

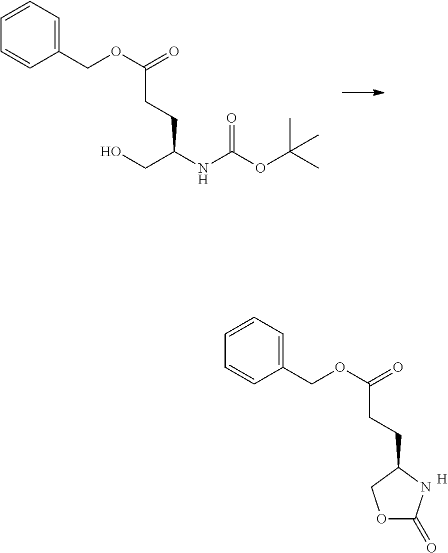

To a solution of benzyl (4R)-4-(tert-butoxycarbonylamino)-5-hydroxy-pentanoate (7.98 g, 24.652 mmol) in dichloroethane (80 mL) was added pyridine (48.900 g, 50 mL, 618.21 mmol). p-toluenesulfonic anhydride (8.65 g, 25.972 mmol) was then added and the mixture was stirred at room temperature for 1 hour and then heated to 90° C. for 2 hours. The mixture was cooled, diluted with dichloromethane (150 mL) and washed with 1N HCl (3×100 mL). The combined organic layers were washed with brine, dried with sodium sulfate and the solvents were removed in vacuo. The residue was purified by silica-gel column chromatography on a 80 g column, eluting from 20% to 80% of EtOAc in heptane to yield benzyl 3-[(4R)-2-oxooxazolidin-4-yl]propanoate (4.85 g, 77%) as a pale brown oil that slowly crystalized over time. ¹H NMR (400 MHz, CDCl₃) δ 7.43-7.30 (m, 5H), 6.15 (br. s., 1H), 5.13 (s, 2H), 4.48 (t, J 8.4 Hz, 1H), 4.02 (dd, J 8.6, 6.1 Hz, 1H), 3.97-3.88 (m, 1H), 2.45 (t, J 7.3 Hz, 2H), 2.00-1.85 (m, 2H). ESI-MS m/z calc. 249.1001, found 250.2 (M+1)⁺; Retention time: 1.511 minutes, LC method X.

### Step 3: (4R)-4-(3-Hydroxy-3-methyl-butyl)oxazolidin-2-one

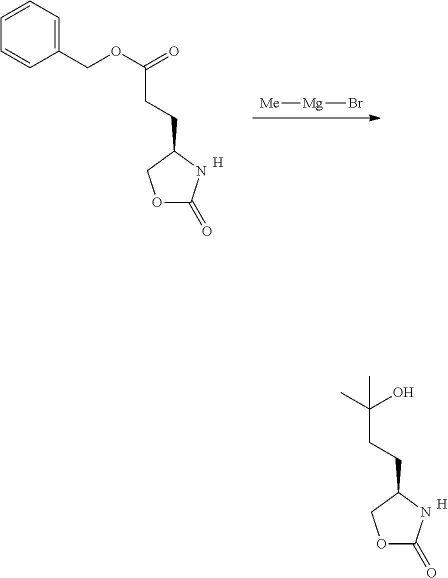

Methylmagnesium bromide (26 mL of 3 M, 78.000 mmol) in diethyl ether was added to a mixture of toluene (42 mL) and tetrahydrofuran (42 mL) at −20° C. (methanol+water+dried ice). A warm tetrahydrofuran (22 mL) solution of benzyl 3-[(4R)-2-oxooxazolidin-4-yl]propanoate (4.85 g, 19.457 mmol) was then added dropwise maintaining the temperature below −10° C. The mixture was warmed up to room temperature and stirred for 2 hours. The reaction mixture was cooled to 0° C., quenched with a 10% aqueous acetic acid solution (50 mL) and the resultant mixture was stirred for 1 hour at room temperature. The layers were separated. The aqueous layer was extracted with methyl-THF (3×100 mL) and then with dichloromethane (2×100 mL). The organic phases were combined, dried on anhydrous sodium sulfate, filtered and concentrated under reduced pressure. The residue was purified by silica-gel column chromatography on a 50 g and 120 g column, eluting from 0 to 15% of isopropanol in dichloromethane to afford (4R)-4-(3-hydroxy-3-methyl-butyl)oxazolidin-2-one (1.73 g, 51%) as a white solid. ¹H NMR (400 MHz, CDCl₃) δ 6.05 (br. s., 1H), 4.50 (t, J 8.4 Hz, 1H), 4.03 (dd, J 8.4, 6.2 Hz, 1H), 3.95-3.81 (m, 1H), 1.76-1.64 (m, 2H), 1.59-1.44 (m, 3H), 1.25 (s, 6H). ESI-MS m/z calc. 173.1052, found 174.2 (M+1)⁺; Retention time: 0.95 minutes, LC method X.

### Step 4: (2R)-2-Amino-5-methyl-hexane-1,5-diol

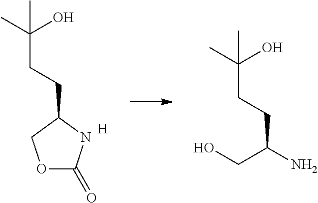

A mixture of (4R)-4-(3-hydroxy-3-methyl-butyl)oxazolidin-2-one (307 mg, 1.7724 mmol), barium hydroxide octahydrate (1.69 g, 5.3572 mmol), ethanol (12 mL) and water (12 mL) was heated at 95° C. to reflux for 2 hours. Reaction mixture was cooled to room temperature before dry ice was slowly added (˜1,8 g) and mixture was stirred vigorously for 2 days. The suspension was filtered over a Celite pad and rinsed with ethanol (˜15 mL). The filtrate was diluted with toluene, co-evaporated three times and concentrated under reduced pressure. Barium salts were observed on the walls of the flask. A minimum of ethanol was added, and the solution was filtered a second time over a Celite pad. The filtrate was concentrated under pressure to provide (2R)-2-amino-5-methyl-hexane-1,5-diol (338.4 mg, 130%) as a yellow oil. The crude was used for the next step without purification. ¹H NMR (400 MHz, DMSO-d₆) δ 3.40-3.28 (m, 1H), 3.25-3.11 (m, 1H), 2.64 (br. s, 1H), 1.81 (s, 2H), 1.51-1.37 (m, 2H), 1.37-1.29 (m, 1H), 1.29-1.18 (m, 1H), 1.06 (d, J 1.0 Hz, 6H). ESI-MS m/z calc. 147.1259, found 148.4 (M+1)⁺; Retention time: 0.22 minutes, LC method X.

### Step 5: 3-[[4-[(2R)-2-Amino-5-hydroxy-5-methyl-hexoxy]-6-(2,6-dimethylphenyl)pyrimidin-2-yl]sulfamoyl]benzoic Acid

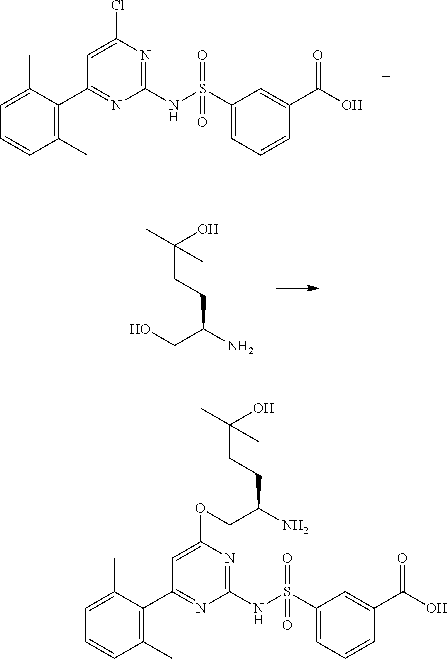

To a solution of 3-[[4-chloro-6-(2,6-dimethylphenyl)pyrimidin-2-yl]sulfamoyl]benzoic acid (371 mg, 0.8878 mmol) and (2R)-2-amino-5-methyl-hexane-1,5-diol (261 mg, 1.7729 mmol) in THE cooled down to 0° C. was slowly added Sodium tert-butoxide (375 mg, 3.9020 mmol). After 2 hours sodium tert-butoxide (76 mg, 0.7908 mmol) was slowly added to the reaction and stirred at room temperature. After 2 hours following the addition, sodium tert-butoxide in THE (200 μL of 2 M, 0.4000 mmol) was slowly added and the reaction was stirred at room temperature overnight. The reaction was partitioned between ethyl acetate (6 mL) and hydrochloric acid 1N (6 mL). The aqueous phase was extracted with ethyl acetate (2×6 mL) and 2-methyltetrahydrofuran (3×6 mL). The organic phases were combined, dried over sodium sulfate, filtered and concentrated to dryness. The solid was triturated with ethyl acetate (10 mL) and the precipitate was filtered then washed with ethyl acetate (2×10 mL) to afford 3-[[4-[(2R)-2-amino-5-hydroxy-5-methyl-hexoxy]-6-(2,6-dimethylphenyl)pyrimidin-2-yl]sulfamoyl]benzoic acid (653.4 mg, 139%, higher mass recovery might be due to salt contamination) as a pale-yellow solid. The crude was used for the next step without purification. ¹H NMR (400 MHz, DMSO-d₆) δ 13.24 (br. s, 1H), 8.43 (s, 1H), 8.19-8.06 (m, 3H), 7.70 (t, J 7.6 Hz, 1H), 7.32-7.19 (m, 1H), 7.18-7.05 (m, 2H), 6.30 (s, 1H), 4.46-4.32 (m, 1H), 4.30-4.18 (m, 1H), 3.53 (s, 1H), 1.99 (s, 6H), 1.78-1.61 (m, 2H), 1.57-1.37 (m, 2H), 1.11 (d, J 7.8 Hz,6H). ESI-MS m/z calc. 528.2043, found 529.2 (M+1)⁺; Retention time: 1.3 minutes, LC method X.

### Step 6: (11R)-6-(2,6-Dimethylphenyl)-11-(3-hydroxy-3-methylbutyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 67)

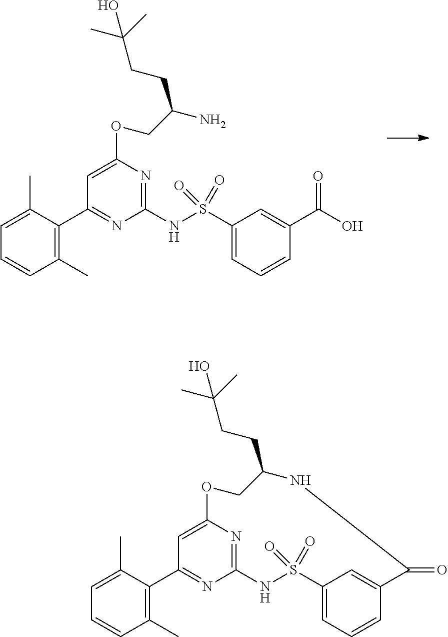 [SMILES: Cc1cccc(C)c1-c1cc(OC[C@H](N)CCC(C)(C)O)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CCC(C)(C)O)CO2]

To a solution of 3-[[4-[(2R)-2-amino-5-hydroxy-5-methyl-hexoxy]-6-(2,6-dimethylphenyl)pyrimidin-2-yl]sulfamoyl]benzoic acid (653.4 mg, 1.2360 mmol) in DMF (14 mL) and EtOAc (46 mL) was added TEA (726.00 mg, 1 mL, 7.1746 mmol). The solution was cooled down to 0° C. and a propylphosphonic anhydride solution (1.2 mL of 50% w/v, 1.8857 mmol) was slowly added. The reaction was stirred overnight at room temperature and then ethyl acetate was removed under vacuo. The product in N,N-Dimethylformamide was diluted in water (20 mL) and extracted with ethyl acetate (3×20 mL). Ethyl acetate was evaporated under vacuo and the resulting mixture was purified by reverse phase chromatography (C₁₈ 50 g) using a gradient of 5% to 100% methanol in water. After purification two different lots were concentrated under vacuo and filtered to provide a first lot of (11R)-6-(2,6-dimethylphenyl)-11-(3-hydroxy-3-methyl-butyl)-2,2-dioxo-9-oxa-2λ⁶-thia-3,5,12,19-tetrazatricyclo[12.3.1.14,8]nonadeca-1(18),4(19),5,7,14,16-hexaen-13-one (125.6 mg, 20%) and a second one of (11R)-6-(2,6-dimethylphenyl)-11-(3-hydroxy-3-methyl-butyl)-2,2-dioxo-9-oxa-2λ⁶-thia-3,5,12,19-tetrazatricyclo[12.3.1.14,8]nonadeca-1(18),4(19),5,7,14,16-hexaen-13-one (158.3 mg, 25%) as a white solid. ¹H NMR (400 MHz, DMSO-d₆) δ 12.27 (br. s., 1H), 8.53 (s, 1H), 7.97-7.89 (m, 1H), 7.68 (d, J 4.6 Hz, 2H), 7.54 (d, J 10.0 Hz, 1H), 7.27-7.21 (m, 1H), 7.11 (d, J 7.6 Hz, 2H), 6.32 (s, 1H), 5.17 (dd, J 10.9, 3.8 Hz, 1H), 3.93 (t, J 11.1 Hz, 1H), 3.72 (br. s., 1H), 3.37-3.23 (m, 1H), 2.05 (s, 6H), 1.73-1.60 (m, 1H), 1.56-1.36 (m, 2H), 1.04-0.97 (m, 1H), 0.94 (d, J 5.6 Hz, 6H) ESI-MS m/z calc. 510.1937, found 511.3 (M+1)⁺; Retention time: 3.11 minutes, LC method Y.

### Example 66: Preparation of Compound 68

### Step 1: (11R)-6-(6,6-Dimethylcyclohexen-1-yl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one

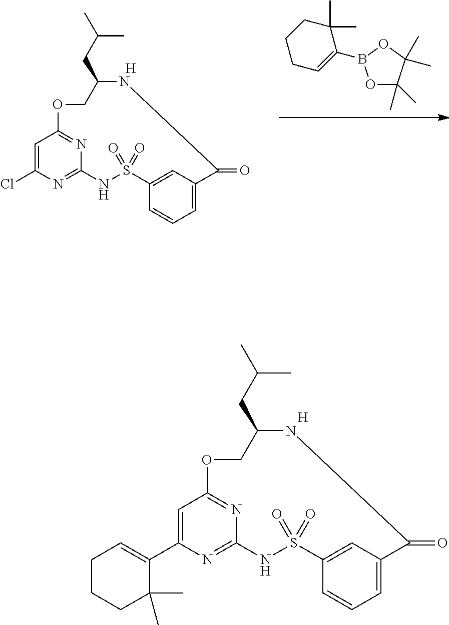

(11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (81 mg, 0.1971 mmol) and 2-(6,6-dimethylcyclohexen-1-yl)-4,4,5,5-tetramethyl-1,3,2-dioxaborolane (116 mg, 0.4912 mmol) were combined in DMSO (1.5 mL). [1,1′-bis(Diphenylphosphino)ferrocene]dichloropalladium(II) (14.5 mg, 0.01982 mmol) and aqueous potassium carbonate (250 μL of 2 M, 0.5000 mmol) were added to the mixture and nitrogen was bubbled through the suspension for 1 minute. The reaction was capped and heated to 120° C. for 4 hours. The reaction mixture was filtered and purified by reverse-phase preparative chromatography utilizing a C₁₈ column a 10-70% over 15 min gradient of acetonitrile in water containing 5 mM HCl to give as a light yellow solid (11R)-6-(6,6-dimethylcyclohexen-1-yl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (15.69 mg, 16%). ESI-MS m/z calc. 484.21442, found 485.2 (M+1)⁺; Retention time: 1.62 minutes, LC method A.

### Step 2: (11R)-6-(2,2-Dimethylcyclohexyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (Compound 68)

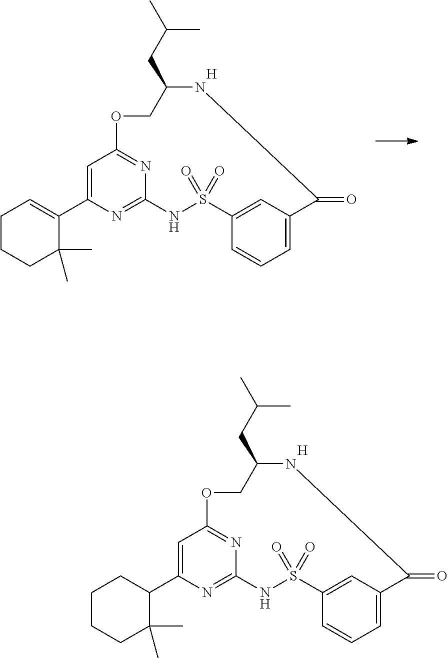

A solution of (11R)-6-(6,6-dimethylcyclohexen-1-yl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (15.69 mg, 0.03238 mmol) in MeOH (600 μL) was purged with nitrogen for 10 minutes, then Pd/C (1.5 mg of 10% w/w, 0.001410 mmol) and a drop of acetic acid (2 μL, 0.03517 mmol) were added followed by a balloon of hydrogen and the mixture was stirred at RT for 16 hours. Additional Pd/C (1.5 mg of 10% w/w, 0.001410 mmol) was added and a recharged balloon of hydrogen and the reaction was stirred at RT for another 60 hours. More palladium hydroxide (2.5 mg, 0.01780 mmol) and a recharged balloon of hydrogen were added and the mixture was stirred at RT for another 24 hours. The mixture was purged with nitrogen for 5 minutes, filtered and evaporated under reduced pressure on high vacuum to afford a white solid which was purified by reverse-phase preparative chromatography utilizing a C₁₈ column and a 10-70% over 30 min gradient of acetonitrile in water containing 5 mM HCl to give as a white solid (11R)-6-(2,2-dimethylcyclohexyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (6.17 mg, 39%). ESI-MS m/z calc. 486.23007, found 487.2 (M+1)⁺; Retention time: 1.6 minutes. ¹H NMR (400 MHz, DMSO-d₆) δ 12.78 (s, 1H), 8.39 (s, 1H), 7.85 (d, J 9.5 Hz, 2H), 7.59 (s, 2H), 6.11 (s, 1H), 5.11 (d, J 11.3 Hz, 1H), 3.79 (t, J 11.0 Hz, 1H), 3.12 (d, J 11.0 Hz, 1H), 2.39-2.31 (m, 1H), 1.74 (dd, J=29.4, 14.1 Hz, 2H), 1.54-1.34 (m, 6H), 1.23-1.08 (m, 3H), 0.86-0.72 (m, 9H), 0.27 (dd, J=27.1, 6.3 Hz, 3H). LC method A.

### Example 67: Preparation of Compound 69

### Step 1: Methyl 2-[[4-chloro-6-(2,6-dimethylphenyl)pyrimidin-2-yl]sulfamoyl]pyridine-4-carboxylate

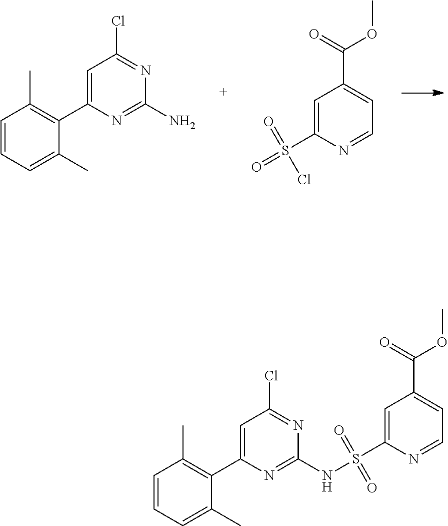 [SMILES: Cc1cccc(C)c1-c1cc(Cl)nc(N)n1 ; COC(=O)c1ccnc(S(=O)(=O)Cl)c1 ; COC(=O)c1ccnc(S(=O)(=O)Nc2nc(Cl)cc(-c3c(C)cccc3C)n2)c1]

Methyl 2-chlorosulfonylpyridine-4-carboxylate (5 g, 21.218 mmol) and 4-chloro-6-(2,6-dimethylphenyl)pyrimidin-2-amine (5 g, 21.395 mmol) were dissolved in anhydrous THE (150 mL) under nitrogen and the solution was cooled to −78 C. A 1M THE solution of LiHMDS (43 mL of 1 M, 43.000 mmol) was added dropwise and the mixture was allowed to warm up gradually to 0° C. The reaction mixture was quenched with saturated aqueous sodium bicarbonate (100 mL) and extracted with chloroform (3×50 mL). The organic fractions were combined, dried over sodium sulfate end evaporated. The residue was purified by silica gel column chromatography using 0-100% hexanes-ethyl acetate to give methyl 2-[[4-chloro-6-(2,6-dimethylphenyl)pyrimidin-2-yl]sulfamoyl]pyridine-4-carboxylate (8.3 g, 80.6%) as a white soldi. ESI-MS m/z calc. 432.06592, found 432.8 (M+1)⁺; Retention time: 5.5 minutes; LC method S.

### Step 2: 2-[[4-Chloro-6-(2,6-dimethylphenyl)pyrimidin-2-yl]sulfamoyl]pyridine-4-Carboxylic Acid

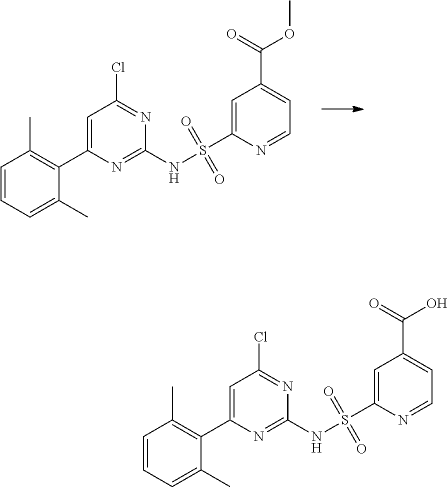 [SMILES: COC(=O)c1ccnc(S(=O)(=O)Nc2nc(Cl)cc(-c3c(C)cccc3C)n2)c1 ; Cc1cccc(C)c1-c1cc(Cl)nc(NS(=O)(=O)c2cc(C(=O)O)ccn2)n1]

A 1M aqueous NaOH solution (95 mL, 95.000 mmol) was added to a solution of methyl 2-[[4-chloro-6-(2,6-dimethylphenyl)pyrimidin-2-yl]sulfamoyl]pyridine-4-carboxylate (8.1 g, 18.712 mmol) in THE (95 mL) and the mixture was stirred at room temperature for 1 hour. 1M aqueous HCl solution was added to pH ˜8 and the mixture was extracted with 2-MeTHF (2×100 mL). The aqueous phase was separated and acidified with 1M aqueous HCl solution to pH-2. The formed precipitate was collected by filtration to give 2-[[4-chloro-6-(2,6-dimethylphenyl)pyrimidin-2-yl]sulfamoyl]pyridine-4-carboxylic acid (5.17 g, 71%) as a white solid. ¹H NMR (250 MHz, DMSO(d₆)) δ 8.87 (d, J 5.0 Hz, 1H), 8.32 (d, J 1.1 Hz, 1H), 8.04 (dt, J 4.9, 1.5 Hz, 1H), 7.32-7.16 (m, 2H), 7.04 (d, J 7.5 Hz, 2H), 1.76 (s, 6H). ESI-MS m/z calc. 418.05026, found 419.3 (M+1)⁺; Retention time: 4.62 minutes; LC method S.

### Step 3: tert-Butyl N-[(1R)-1-(cyclohexylmethyl)-2-hydroxy-ethyl]carbamate

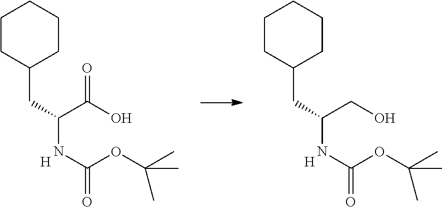

A THE (40 mL) solution of (2R)-2-(tert-butoxycarbonylamino)-3-cyclohexyl-propanoic acid (5.03 g, 18.54 mmol) was cooled to 0° C. and was treated with borane-THF (50 mL of 1 M, 50.00 mmol) over 20 min. The reaction mixture was then warmed to room temperature and then stirred for 2 h. The reaction mixture was cooled to −10° C. and carefully quenched with MeOH (15 mL, 370.3 mmol) (Caution: gas evolution) and slowly warmed to room temperature over 30 minutes and then concentrated in vacuo to give tert-butyl N-[(1R)-1-(cyclohexylmethyl)-2-hydroxy-ethyl]carbamate (4.93 g, 103%) ESI-MS m/z calc. 257.1991, found 158.1 (M+1)⁺; Retention time: 0.66 minutes. (M-Boc) LC method D.

### Step 4: 2-[[4-[(2R)-2-Amino-3-cyclohexyl-propoxy]-6-(2,6-dimethylphenyl)pyrimidin-2-yl]sulfamoyl]pyridine-4-carboxylic Acid

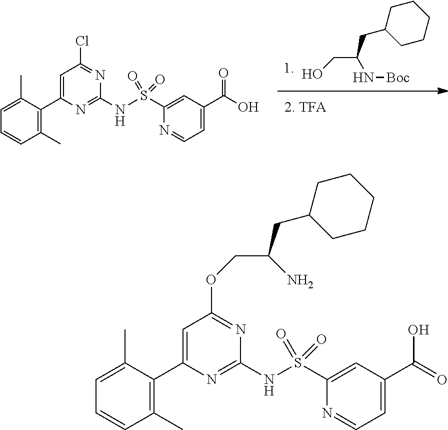 [SMILES: Cc1cccc(C)c1-c1cc(Cl)nc(NS(=O)(=O)c2cc(C(=O)O)ccn2)n1 ; CC(C)(C)OC(=O)N[C@@H](CO)CC1CCCCC1 ; Cc1cccc(C)c1-c1cc(OC[C@H](N)CC2CCCCC2)nc(NS(=O)(=O)c2cc(C(=O)O)ccn2)n1]

To a solution of tert-butyl N-[(1R)-1-(cyclohexylmethyl)-2-hydroxy-ethyl]carbamate (86 mg, 0.3342 mmol) and 2-[[4-chloro-6-(2,6-dimethylphenyl)pyrimidin-2-yl]sulfamoyl]pyridine-4-carboxylic acid (100 mg, 0.2387 mmol) in THE (1.1 mL) was added Potassium tert-butoxide (107 mg, 0.9536 mmol) at 23° C. The reaction was stirred for 16 h and then quenched with Trifluoroacetic acid (110 μL, 1.428 mmol). The volatiles were removed under a slow stream of air The crude residue was dissolved in DCM (1.1 mL) and trifluoroacetic acid (1,100 μL, 14.28 mmol). The reaction was stirred for 15 min and then the volatiles were removed under a steady stream of air. The sample was purified by reverse phase HPLC (Waters Sunfire C₁₈ column (100×50 mm, 10 m particle size), gradient: 1-99% acetonitrile in water (5 mM HCl) over 15.0 minutes) to give 2-[[4-[(2R)-2-amino-3-cyclohexyl-propoxy]-6-(2,6-dimethylphenyl)pyrimidin-2-yl]sulfamoyl]pyridine-4-carboxylic acid (10.3 mg, 6%) as a white solid. ESI-MS m/z calc. 539.2202, found 540.32 (M+1)⁺; Retention time: 0.47 minutes, LC method D.

### Step 5: (11R)-11-(Cyclohexylmethyl)-6-(2,6-dimethylphenyl)-2,2-dioxo-9-oxa-2λ⁶-thia-3,5,12,17,19-pentazatricyclo[12.3.1.14,8]nonadeca-1(17),4(19),5,7,14(18),15-hexaen-13-one (Compound 69)

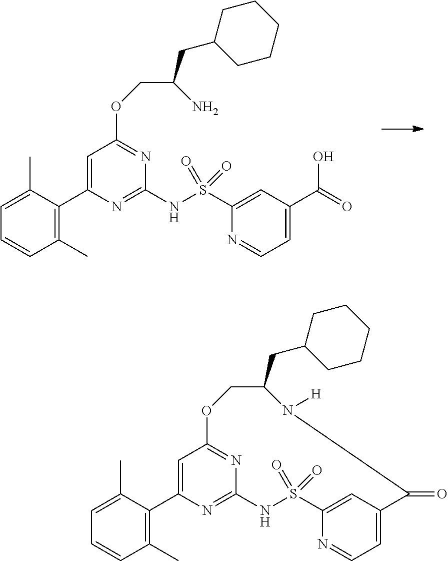 [SMILES: Cc1cccc(C)c1-c1cc(OC[C@H](N)CC2CCCCC2)nc(NS(=O)(=O)c2cc(C(=O)O)ccn2)n1 ; Cc1cccc(C)c1-c1cc2nc(n1)NS(=O)(=O)c1cc(ccn1)C(=O)N[C@H](CC1CCCCC1)CO2]

To a solution of 2-[[4-[(2R)-2-amino-3-cyclohexyl-propoxy]-6-(2,6-dimethylphenyl)pyrimidin-2-yl]sulfamoyl]pyridine-4-carboxylic acid (10.3 mg, 0.01527 mmol) in DMF (0.5 mL) was added [dimethylamino(triazolo[4,5-b]pyridin-3-yloxy)methylene]-dimethyl-ammonium; hexafluorophosphate (5.8 mg, 0.01525 mmol). After 5 min, triethylamine (8 mg, 0.07906 mmol) was added and the reaction was further stirred for 15 min. The sample was purified by reverse phase HPLC (Phenomenex Luna C₁₈ column (75×30 mm, 5 m particle size), gradient: 1-99% acetonitrile in water (5 mM HCl) over 15.0 minutes) which afforded (11R)-11-(cyclohexylmethyl)-6-(2,6-dimethylphenyl)-2,2-dioxo-9-oxa-2λ⁶-thia-3,5,12,17,19-pentazatricyclo[12.3.1.14,8]nonadeca-1(17),4(19),5,7,14(18),15-hexaen-13-one (1.3 mg, 16%) as a white solid. ESI-MS m/z calc. 521.20966, found 522.35 (M+1)⁺; Retention time: 1.67 minutes, LC method A.

### Example 68: Preparation of Compound 70

### Step 1: Methyl 6-[[4-chloro-6-(2,6-dimethylphenyl)pyrimidin-2-yl]sulfamoyl]pyrazine-2-carboxylate

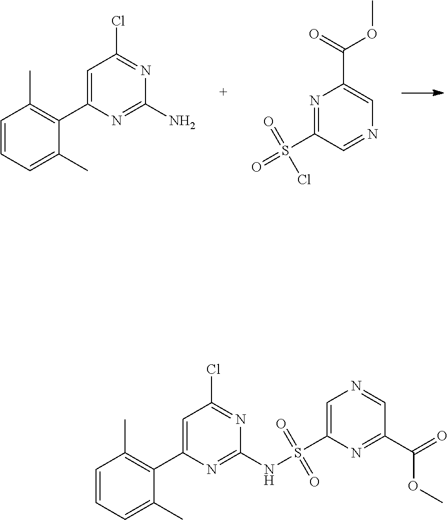 [SMILES: Cc1cccc(C)c1-c1cc(Cl)nc(N)n1 ; COC(=O)c1cncc(S(=O)(=O)Cl)n1 ; COC(=O)c1cncc(S(=O)(=O)Nc2nc(Cl)cc(-c3c(C)cccc3C)n2)n1]

4-Chloro-6-(2,6-dimethylphenyl)pyrimidin-2-amine (4.15 g, 17.758 mmol) was dissolved in MeTHF (25 mL) and was cooled in an iced bath. Methyl 6-chlorosulfonylpyrazine-2-carboxylate (13.64 g, 57.642 mmol) in MeTHF (25 mL) was added at 0° C. To the cold solution, lithium tert-butoxide (17 mL of 3.1 M, 52.700 mmol) (in heptane) was added dropwise. The ice bath was removed, and the mixture was stirred for 3 hours at room temperature. 1N aqueous hydrochloric acid solution (50 mL) was added and the phases was separated. The aqueous phase was extracted with MeTHF (50 mL) and the organic phase were combined, washed with brine (50 mL), dried over sodium sulfate, filtered and concentrated. The residue was purified by silica-gel column chromatography on a 330 g column, eluting from 0% to 30% of ethyl acetate in heptanes to afford methyl 6-[[4-chloro-6-(2,6-dimethylphenyl)pyrimidin-2-yl]sulfamoyl]pyrazine-2-carboxylate (4.85 g, 18%) as an off-white solid. ¹H NMR (300 MHz, CDCl₃) δ 9.58 (s, 1H), 9.44 (s, 1H), 7.23-7.17 (m, 1H), 7.06 (d, J 7.9 Hz, 2H), 6.91 (s, 1H), 4.03 (s, 3H), 1.95 (s, 6H). ESI-MS m/z calc. 433.06116, found 434.1 (M+1)⁺; Retention time: 1.98 minutes; LC method K.

### Step 2: 6-[[4-Chloro-6-(2,6-dimethylphenyl)pyrimidin-2-yl]sulfamoyl]pyrazine-2-carboxylic Acid

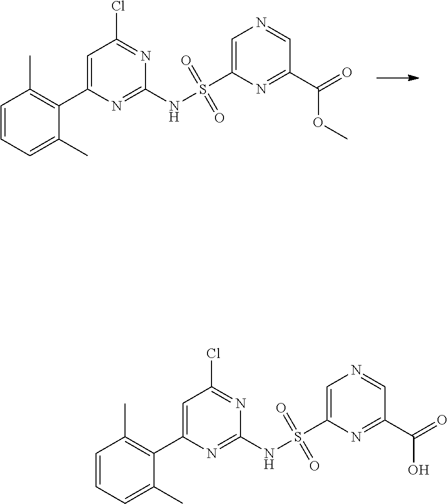 [SMILES: COC(=O)c1cncc(S(=O)(=O)Nc2nc(Cl)cc(-c3c(C)cccc3C)n2)n1 ; Cc1cccc(C)c1-c1cc(Cl)nc(NS(=O)(=O)c2cncc(C(=O)O)n2)n1]

A mixture of methyl 6-[[4-chloro-6-(2,6-dimethylphenyl)pyrimidin-2-yl]sulfamoyl]pyrazine-2-carboxylate (4.85 g, 10.136 mmol) in THE (125 mL) and water (125 mL) was treated with lithium hydroxide mono hydrate (1.3 g, 30.979 mmol) and stirred vigorously at room temperature for 3 hours. 1N Aqueous sodium hydroxide solution (125 mL) was added and extracted with diethyl ether (125 mL) and 2-MeTHF (125 mL). The aqueous phase was acidified to pH<3 with 3N aqueous hydrochloric acid solution and extracted with ethyl acetate (3×125 mL). The combined organic layers were washed with brine (125 mL), dried over sodium sulfate, filtered and concentrated under reduced pressure to afford 6-[[4-chloro-6-(2,6-dimethylphenyl)pyrimidin-2-yl]sulfamoyl]pyrazine-2-carboxylic acid (4.4 g, 87%) as a yellow solid. ¹H NMR (300 MHz, DMSO-d₆) δ 13.55-12.73 (m, 2H), 9.34 (s, 1H), 9.32 (s, 1H), 7.30 (s, 1H), 7.26-7.16 (m, 1H), 7.07 (d, J 7.6 Hz, 2H), 1.82 (s, 6H). ESI-MS m/z calc. 419.0455, found 420.1 (M+1)⁺; Retention time: 2.59 minutes; LC method U.

### Step 3: 6-[[4-[(2R)-2-Amino-4-methyl-pentoxy]-6-(2,6-dimethylphenyl)pyrimidin-2-yl]sulfamoyl]pyrazine-2-carboxylic Acid

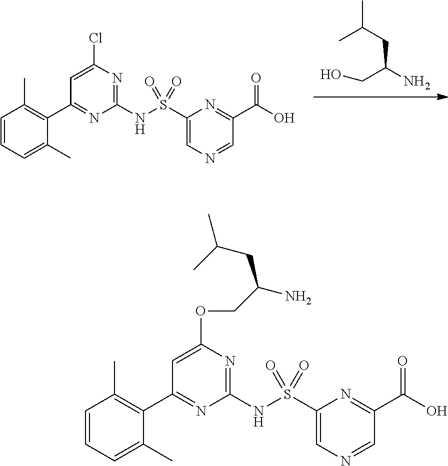

In a 20 mL vial, 6-[[4-chloro-6-(2,6-dimethylphenyl)pyrimidin-2-yl]sulfamoyl]pyrazine-2-carboxylic acid (218 mg, 0.5192 mmol) and (2R)-2-amino-4-methyl-pentan-1-ol (65 mg, 0.5547 mmol) were dissolved under nitrogen in anhydrous THE (1.2 mL). Sodium tert-butoxide (200 mg, 2.081 mmol) was added (slight exotherm). The reaction turned quickly into a thick gel, therefore more THF (1 mL) was added. After stirring for 1.5 hours at room temperature (over 90% conversion), the mixture was partitioned between ethyl acetate (30 mL) and aqueous 1M HCl (30 mL). After separation, the aqueous phase was further extracted with EtOAc. The combined extracts were dried over sodium sulfate and the solvents evaporated to give 140 mg of crude material. Significant amount of product was still detected in the aqueous phase. Brine (30 mL) was added and further EtOAc extraction was carried out. After drying over sodium sulfate, the organic phase was combined with the rest of the material and evaporated to give a total of 220 mg of crude material. It was dissolved in DMSO and purified by reverse phase preparative HPLC (C₁₈) using a gradient of acetonitrile in water (1 to 99% over 15 min) and HCl as a modifier. Evaporation of the solvents gave 6-[[4-[(2R)-2-amino-4-methyl-pentoxy]-6-(2,6-dimethylphenyl)pyrimidin-2-yl]sulfamoyl]pyrazine-2-carboxylic Acid (hydrochloride salt) (90 mg, 32%) as an off-white solid. ESI-MS m/z calc. 500.18417, found 501.3 (M+1)⁺; Retention time: 0.96 minutes (LC method A).

### Step 4: (11R)-6-(2,6-Dimethylphenyl)-11-isobutyl-2,2-dioxo-9-oxa-2⁶-thia-3,5,12,16,18,19-hexazatricyclo[12.3.1.14,8]nonadeca-1(18),4(19),5,7,14,16-hexaen-13-one (Compound 70)

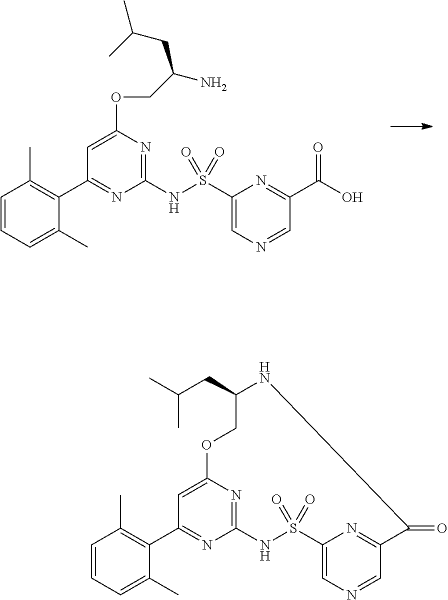 [SMILES: Cc1cccc(C)c1-c1cc(OC[C@H](N)CC(C)C)nc(NS(=O)(=O)c2cncc(C(=O)O)n2)n1 ; Cc1cccc(C)c1-c1cc2nc(n1)NS(=O)(=O)c1cncc(n1)C(=O)N[C@H](CC(C)C)CO2]

A 100 mL flask was charged under nitrogen with 6-[[4-[(2R)-2-amino-4-methyl-pentoxy]-6-(2,6-dimethylphenyl)pyrimidin-2-yl]sulfamoyl]pyrazine-2-carboxylic Acid (hydrochloride salt) (90 mg, 0.1676 mmol), HATU (133 mg, 0.3498 mmol), anhydrous DMF (9 mL) and DIEA (163 μL, 0.9358 mmol). The mixture was stirred at room temperature for 25 hours. It was concentrated and diluted with DMSO (2 mL). The solution was microfiltered through a Whatman 0.45 uM PTFE syringe filter disc and purified by reverse phase preparative HPLC (C₁₈) using a gradient of acetonitrile in water (1 to 99% over 15 min) and HCl as a modifier to give (11R)-6-(2,6-dimethylphenyl)-11-isobutyl-2,2-dioxo-9-oxa-2λ⁶-thia-3,5,12,16,18,19-hexazatricyclo[12.3.1.14,8]nonadeca-1(18),4(19),5,7,14,16-hexaen-13-one (24 mg, 29%) as an off-white solid. ESI-MS m/z calc. 482.1736, found 483.29 (M+1)⁺; Retention time: 1.42 minutes (LC method A). ¹H NMR (500 MHz, DMSO-d₆)δ 13.24 (broad s, 1H), 9.24 (s, 1H), 9.05 (s, 1H), 8.36 (d, J 9.6 Hz, 1H), 7.29 (t, J 7.6 Hz, 1H), 7.15 (d, J 7.7 Hz, 2H), 6.40 (s, 1H), 5.69 (dd, J 9.8, 4.2 Hz, 1H), 3.75 (t, J 10.4 Hz, 1H), 3.34-3.23 (m, 1H overlapped with water), 2.16 (br s, 3H), 2.03 (br s, 3H), 1.70-1.57 (m, 1H), 1.51 (ddd, J 14.3, 10.6, 3.9 Hz, 1H), 1.28 (ddd, J 13.3, 9.9, 2.8 Hz, 1H), 0.81 (d, J 6.7 Hz, 3H), 0.45 (d, J 6.5 Hz, 3H).

### Example 69: Preparation of Compound 71 and Compound 72

### Step 1: 2-[[4-Chloro-6-(2,6-dimethylphenyl)pyrimidin-2-yl]sulfamoyl]cyclobutanecarboxylic Acid

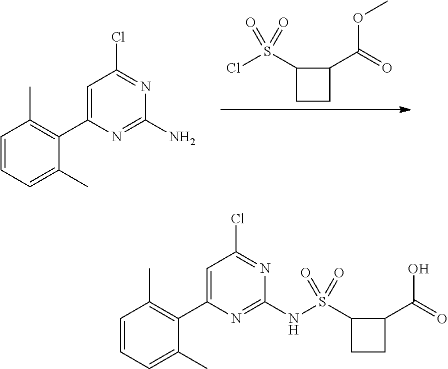 [SMILES: Cc1cccc(C)c1-c1cc(Cl)nc(N)n1 ; COC(=O)C1CCC1S(=O)(=O)Cl ; Cc1cccc(C)c1-c1cc(Cl)nc(NS(=O)(=O)C2CCC2C(=O)O)n1]

In a 50-mL round-bottomed flask, HMDS (5.0 mL, 23.70 mmol) was dissolved in THE (10 mL) and cooled to −78° C. A hexanes solution of n-BuLi (9.5 mL of 2.5 M, 23.75 mmol) was added in one portion, and the resulting mixture was warmed to room temperature over 15 min. In a separate 500-mL round-bottomed flask, 4-chloro-6-(2,6-dimethylphenyl)pyrimidin-2-amine (2.8024 g, 11.52 mmol) was dissolved in THE (120 mL), to which methyl 2-chlorosulfonylcyclobutanecarboxylate (2.4988 g, 11.75 mmol) was added. The resulting mixture was cooled to −78° C., upon which the above-prepared LiHMDS solution was added dropwise via a syringe. The solution was stirred at −78° C. for 15 min, and warmed to room temperature over 45 min. It was then quenched with 0.5 N HCl solution (120 mL), and stirred for 10 min. The resulting mixture was extracted with ethyl acetate (3×100 mL). The combined organic extracts were washed with water (200 mL) and saturated aqueous sodium chloride solution (200 mL), then dried over sodium sulfate, filtered, and evaporated in vacuo to give 4.3 g of a yellow foam. Purification by silica gel chromatography (120 g of silica) using a gradient eluent of 1 to 40% ethyl acetate in hexanes gave an off-white foam, methyl 2-[[4-chloro-6-(2,6-dimethylphenyl)pyrimidin-2-yl]sulfamoyl]cyclobutanecarboxylate (0.900 g, 19%) ESI-MS m/z calc. 409.0863, found 410.1 (M+1)⁺; Retention time: 1.68 minutes (LC method A).

In a 100-mL round-bottomed flask, the product from above (0.900 g, 2.1956 mmol) was dissolved in THE (15 mL), to which aqueous NaOH (15 mL of 1.0 M, 15.00 mmol) was added. The resulting mixture was stirred at room temperature for 1 h, after which it was quenched with 1 N HCl solution (30 mL). This mixture was diluted with EtOAc (200 mL); the phases were vigorously mixed and then allowed to settle into two layers. The aqueous layer was discarded; the organic layer was washed with water (100 mL) and saturated aqueous sodium chloride solution (100 mL), then dried over sodium sulfate, filtered, and evaporated in vacuo. The obtained solid was re-dissolved in THE (30 mL) and re-evaporated to dryness. A light yellow foam resulted: 2-[[4-chloro-6-(2,6-dimethylphenyl)pyrimidin-2-yl]sulfamoyl]cyclobutanecarboxylic acid (0.858 g, 19%)¹H NMR (499 MHz, dimethylsulfoxide-d₆) δ 12.44 (s, 1H), 11.71 (s, 1H), 7.33 (s, 1H), 7.26 (t, J 7.7 Hz, 1H), 7.14 (d, J 7.5 Hz, 2H), 4.58 (q, J 8.6 Hz, 1H), 3.40 (q, J 8.9 Hz, 1H), 2.38-2.27 (m, 1H), 2.24-2.14 (m, 2H), 2.07 (s, 6H), 2.06-2.02 (m, 1H) ESI-MS m/z calc. 395.07065, found 396.1 (M+1)⁺; Retention time: 1.43 minutes (LC method A).

### Step 2: (10R)-15-(2,6-Dimethylphenyl)-10-isobutyl-3,3-dioxo-12-oxa-3λ⁶-thia-2,9,16,17-tetrazatricyclo[11.3.1.04,7]heptadeca-1(17),13,15-trien-8-one, diastereomer 1 (Compound 72), and (10R)-15-(2,6-dimethylphenyl)-10-isobutyl-3,3-dioxo-12-oxa-3λ⁶-thia-2,9,16,17-tetrazatricyclo[11.3.1.04,7]heptadeca-1(17),13,15-trien-8-one, diastereomer 2 (Compound 71)

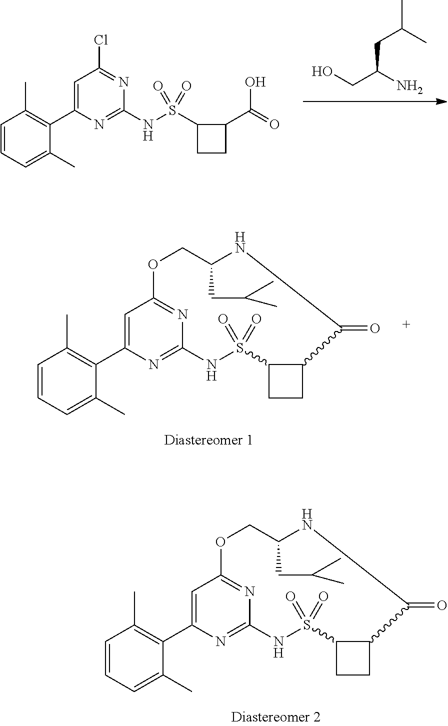

In a 20-mL vial, 2-[[4-chloro-6-(2,6-dimethylphenyl)pyrimidin-2-yl]sulfamoyl]cyclobutanecarboxylic acid (140.5 mg, 0.3549 mmol) was dissolved in THE (4.0 mL), to which (2R)-2-amino-4-methyl-pentan-1-ol (64.2 mg, 0.5478 mmol) and NaOtBu (209.8 mg, 2.183 mmol) were added. This mixture was stirred at room temperature for 1 h. After this time, 1 N HCl solution (4.0 mL) was added, followed by EtOAc (3.0 mL). The phases were vigorously mixed and then allowed to settle into two layers. The organic layer was filtered and purified by reverse phase preparative chromatography using a C₁₈ column and a gradient eluent of 1 to 70% acetonitrile in water containing 5 mM hydrochloric acid to give 2-[[4-[(2R)-2-amino-4-methyl-pentoxy]-6-(2,6-dimethylphenyl)pyrimidin-2-yl]sulfamoyl]cyclobutanecarboxylic acid (hydrochloride salt) (88.1 mg, 48%) ESI-MS m/z calc. 476.20935, found 477.2 (M+1)⁺; Retention time: 1.06 minutes (LC method A).

In a 20-mL vial, the product from above (88.1 mg, 0.1717 mmol) was dissolved in DMF (4.0 mL), to which DIPEA (0.5 mL, 2.871 mmol) and HATU (90.5 mg, 0.2380 mmol) were added. The resulting mixture was stirred at room temperature for 5 min. This mixture was then filtered and purified by reverse phase preparative chromatography using a C₁₈ column and a gradient eluent of 1 to 70% acetonitrile in water containing 5 mM hydrochloric acid to give two diastereomers: diastereomer 1, (10R)-15-(2,6-dimethylphenyl)-10-isobutyl-3,3-dioxo-12-oxa-3λ⁶-thia-2,9,16,17-tetrazatricyclo[11.3.1.04,7]heptadeca-1(17),13,15-trien-8-one (13.7 mg, 8%) ESI-MS m/z calc. 458.19876, found 459.2 (M+1)⁺; Retention time: 1.51 minutes; and diastereomer 2, (10R)-15-(2,6-dimethylphenyl)-10-isobutyl-3,3-dioxo-12-oxa-3λ⁶-thia-2,9,16,17-tetrazatricyclo[11.3.1.04,7]heptadeca-1(17),13,15-trien-8-one (6.8 mg, 4%) ESI-MS m z calc. 458.19876, found 459.2 (M+1)⁺; Retention time: 1.59 minutes, LC method A.

### Example 70: Preparation of Compound 73

### Step 1: (2R)-2-Amino-3-cyclohexyl-propan-1-ol

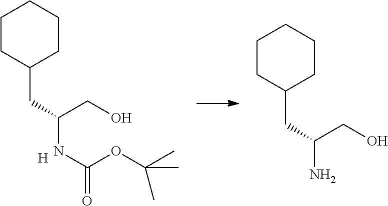

A dioxane (2 mL) solution of tert-butyl N-[(1R)-1-(cyclohexylmethyl)-2-hydroxy-ethyl]carbamate (0.669 g, 2.599 mmol) is treated with HCl (4000 μL of 4 M, 16.00 mmol) and stirred for 2 h at room temperature. The mixture was concentrated in vacuo to give (2R)-2-amino-3-cyclohexyl-propan-1-ol (hydrochloride salt) (512.3 mg, 102%) ESI-MS m/z calc. 157.14667, found 158.1 (M+1)⁺; Retention time: 0.66 minutes; LC method D.

### Step 2: Ethyl 1-chlorosulfonylpiperidine-3-carboxylate

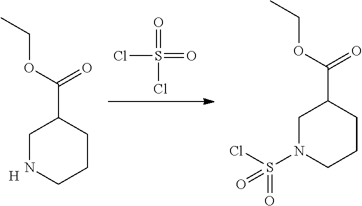

To a solution of sulfuryl chloride (1.7 mL, 21.00 mmol) in DCM (19 mL) was added dropwise a solution of ethyl piperidine-3-carboxylate (3.0 g, 19.08 mmol) and N,N-dimethylpyridin-4-amine (2.6 g, 21.28 mmol) in DCM (19 mL) over 30 min at −78° C. The solution was allowed to stir at 23° C. for 3 h. The reaction mixture was washed with a solution of aqueous hydrochloric acid (1M) and then brine. The organic layer was dried over magnesium sulfate, filtered, and concentrated in vacuo. The crude residue was separated by flash column chromatography on silica gel (gradient: 5 to 50% ethyl acetate in hexanes) which afforded ethyl 1-chlorosulfonylpiperidine-3-carboxylate (3.27 g, 67%) as a clear oil. ¹H NMR (400 MHz, Chloroform-d) δ 4.19 (qd, J=7.1, 1.3 Hz, 2H), 3.89 (d, J 12.1 Hz, 1H), 3.69 (d, J 11.7 Hz, 1H), 3.12 (s, 1H), 2.94 (s, 1H), 2.74 (tt, J 10.1, 4.0 Hz, 1H), 2.08 (dd, J 13.7, 4.2 Hz, 1H), 1.95 (dtd, J=13.3, 4.5, 3.3 Hz, 1H), 1.78 (dtt, J=13.6, 10.6, 3.9 Hz, 1H), 1.72-1.61 (m, 1H), 1.28 (t, J 7.1 Hz, 3H).

### Step 3: (2R)-3-Cyclohexyl-2-(dibenzylamino)propan-1-ol

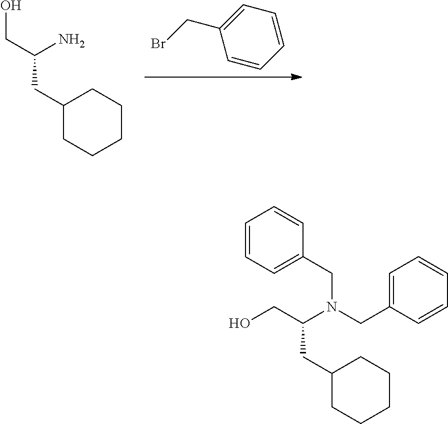 [SMILES: N[C@@H](CO)CC1CCCCC1 ; BrCc1ccccc1 ; OC[C@@H](CC1CCCCC1)N(Cc1ccccc1)Cc1ccccc1]

In a 250-mL round-bottomed flask, (2R)-2-amino-3-cyclohexyl-propan-1-ol (2.97 g, 18.89 mmol) and potassium carbonate (6.5 g, 47.03 mmol) were mixed with water (10 mL) and EtOH (35 mL). A solution of benzyl bromide (6.5 g, 38.00 mmol) in EtOH (5 mL) was added, and then the reaction mixture was stirred vigorously at room temperature for 36 h, after which it was filtered and evaporated to dryness in vacuo. This crude oil was purified by silica gel chromatography using a gradient eluent of 0 to 5% methanol in dichloromethane, and evaporated under high vacuum while stirring to give a yellow transparent viscous liquid, (2R)-3-cyclohexyl-2-(dibenzylamino)propan-1-ol (4.0202 g, 63%) ¹H NMR (400 MHz, dimethylsulfoxide-d₆) δ 7.37-7.26 (m, 8H), 7.25-7.16 (m, 2H), 4.42-4.31 (m, 1H, D₂O exchangeable), 3.72-3.56 (m, 5H), 3.43-3.35 (m, 1H), 2.67-2.55 (m, 1H), 1.68-1.28 (m, 6H), 1.26-0.98 (m, 5H), 0.89-0.76 (m, 1H), 0.63-0.48 (m, 1H) ESI-MS m/z calc. 337.24057, found 338.4 (M+1)⁺; Retention time: 1.38 minutes, LC method A.

### Step 4: Ethyl 1-sulfamoylpiperidine-3-carboxylate

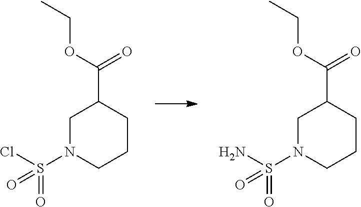

To a solution of ethyl 1-chlorosulfonylpiperidine-3-carboxylate (1 g, 3.911 mmol) in Ethanol (3.911 mL) was added ammonium hydroxide (approximately 5.482 mL of 30% w/v, 46.93 mmol). The reaction was stirred for 3 h and then concentrated in vacuo. The crude residue was used directly in the next reaction.

### Step 5: (2R)—N,N-Dibenzyl-1-[2-chloro-6-(2,6-dimethylphenyl)pyrimidin-4-yl]oxy-3-cyclohexyl-propan-2-amine

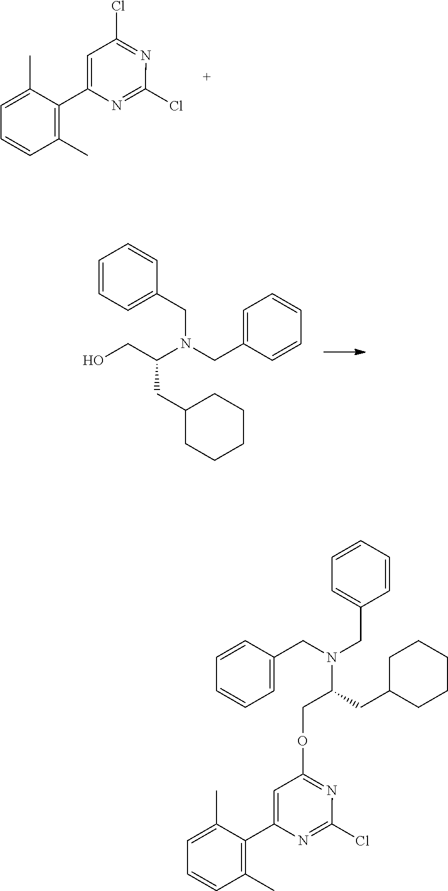 [SMILES: Cc1cccc(C)c1-c1cc(Cl)nc(Cl)n1 ; OC[C@@H](CC1CCCCC1)N(Cc1ccccc1)Cc1ccccc1 ; Cc1cccc(C)c1-c1cc(OC[C@@H](CC2CCCCC2)N(Cc2ccccc2)Cc2ccccc2)nc(Cl)n1]

To a solution of 2,4-dichloro-6-(2,6-dimethylphenyl)pyrimidine (100 mg, 0.3951 mmol) and (2R)-3-cyclohexyl-2-(dibenzylamino)propan-1-ol (160 mg, 0.4741 mmol) in NMP (800 μL) was added potassium tert-butoxide (53 mg, 0.4723 mmol) at 0° C. The reaction was allowed to warm to 23° C. over 24 h. The sample was purified by reverse phase HPLC (Phenomenex Luna C₁₈ column (75×30 mm, 5 m particle size), gradient: 1-99% acetonitrile in water (5 mM HCl) over 15.0 minutes) which afforded (2R)—N,N-dibenzyl-1-[2-chloro-6-(2,6-dimethylphenyl)pyrimidin-4-yl]oxy-3-cyclohexyl-propan-2-amine (40 mg, 13%) as a clear oil. ESI-MS m/z calc. 553.286, found 554.4 (M+1)⁺; Retention time: 0.87 minutes, LC method D.

### Step 6: Ethyl 1-[[4-[(2R)-3-cyclohexyl-2-(dibenzylamino)propoxy]-6-(2,6-dimethylphenyl)pyrimidin-2-yl]sulfamoyl]piperidine-3-carboxylate

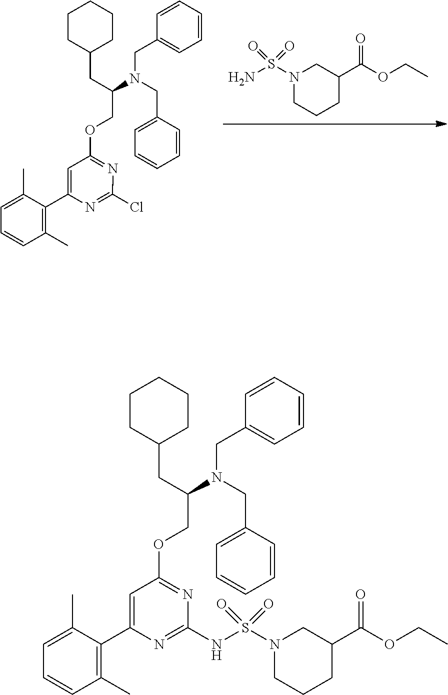 [SMILES: Cc1cccc(C)c1-c1cc(OC[C@@H](CC2CCCCC2)N(Cc2ccccc2)Cc2ccccc2)nc(Cl)n1 ; CCOC(=O)C1CCCN(S(N)(=O)=O)C1 ; CCOC(=O)C1CCCN(S(=O)(=O)Nc2nc(OC[C@@H](CC3CCCCC3)N(Cc3ccccc3)Cc3ccccc3)cc(-c3c(C)cccc3C)n2)C1]

To a 0.5 mL microwave tube was added cesium carbonate (35 mg, 0.1074 mmol). A solution of (2R)—N,N-dibenzyl-1-[2-chloro-6-(2,6-dimethylphenyl)pyrimidin-4-yl]oxy-3-cyclohexyl-propan-2-amine (20 mg, 0.03609 mmol) Xantphos (9 mg, 0.01555 mmol) Pd(OAc)2 (1.8 mg, 0.008017 mmol) in dioxane (0.4 mL) was added to the vial followed by ethyl 1-sulfamoylpiperidine-3-carboxylate (25 mg, 0.1058 mmol). The reaction mixture was purged with nitrogen for 1 min, sealed and reacted at 125° C. for 25 min. The resulting mixture was filtered and purified by preparative reverse phase HPLC (C₁₈): 1-99% ACN in water/HCl modifier (15 min) to afford ethyl 1-[[4-[(2R)-3-cyclohexyl-2-(dibenzylamino)propoxy]-6-(2,6-dimethylphenyl)pyrimidin-2-yl]sulfamoyl]piperidine-3-carboxylate (hydrochloride salt) (6.8 mg, 23%). ESI-MS m/z calc. 753.3924, found 754.58 (M+1)⁺; Retention time: 1.08 minutes, LC method A, 50-99% gradient.

### Step 7: 1-[[4-[(2R)-3-Cyclohexyl-2-(dibenzylamino)propoxy]-6-(2,6-dimethylphenyl)pyrimidin-2-yl]sulfamoyl]piperidine-3-carboxylic Acid

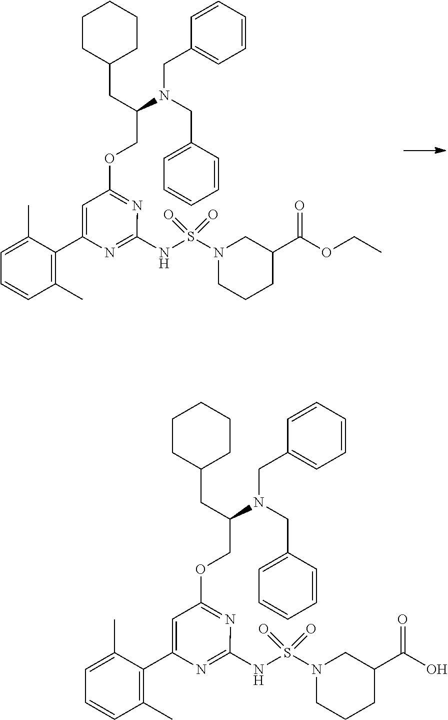 [SMILES: CCOC(=O)C1CCCN(S(=O)(=O)Nc2nc(OC[C@@H](CC3CCCCC3)N(Cc3ccccc3)Cc3ccccc3)cc(-c3c(C)cccc3C)n2)C1 ; Cc1cccc(C)c1-c1cc(OC[C@@H](CC2CCCCC2)N(Cc2ccccc2)Cc2ccccc2)nc(NS(=O)(=O)N2CCCC(C(=O)O)C2)n1]

In a 4 mL vial, ethyl 1-[[4-[(2R)-3-cyclohexyl-2-(dibenzylamino)propoxy]-6-(2,6-dimethylphenyl)pyrimidin-2-yl]sulfamoyl]piperidine-3-carboxylate (6.8 mg, 0.009019 mmol) was dissolved in MeOH (0.4 mL) and NaOH (100 μL of 6 M, 0.6000 mmol) was added followed by THE (0.1 mL). The reaction was stirred for 2 h, after which the solution was diluted with 20 mL water, acidified to pH 7 with 6 M HCl and extracted 3× with 15 mL EtOAc. The organic layer was collected, dried over magnesium sulfate and evaporated to afford 1-[[4-[(2R)-3-cyclohexyl-2-(dibenzylamino)propoxy]-6-(2,6-dimethylphenyl)pyrimidin-2-yl]sulfamoyl]piperidine-3-carboxylic acid (5.3 mg, 81%) ESI-MS m/z calc. 725.3611, found 726.52 (M+1)⁺; Retention time: 0.68 minutes, LC method A, 50-99% gradient.

### Step 8: 1-[[4-[(2R)-2-Amino-3-cyclohexyl-propoxy]-6-(2,6-dimethylphenyl)pyrimidin-2-yl]sulfamoyl]piperidine-3-carboxylic Acid

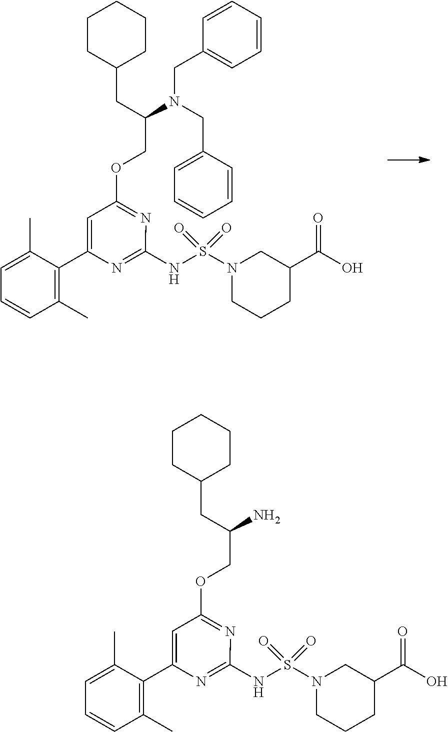 [SMILES: Cc1cccc(C)c1-c1cc(OC[C@@H](CC2CCCCC2)N(Cc2ccccc2)Cc2ccccc2)nc(NS(=O)(=O)N2CCCC(C(=O)O)C2)n1 ; Cc1cccc(C)c1-c1cc(OC[C@H](N)CC2CCCCC2)nc(NS(=O)(=O)N2CCCC(C(=O)O)C2)n1]

In a 4 mL vial, 1-[[4-[(2R)-3-cyclohexyl-2-(dibenzylamino)propoxy]-6-(2,6-dimethylphenyl)pyrimidin-2-yl]sulfamoyl]piperidine-3-carboxylic acid (8.3 mg, 0.01143 mmol) was dissolved in MeOH (0.6 mL) and Pd/C (15 mg of 10% w/w, 0.01410 mmol) was added. The mixture was stirred for 1 h under hydrogen (1 L, 0.000E-1 mmol) (balloon), filtered and evaporated to yield 1-[[4-[(2R)-2-amino-3-cyclohexyl-propoxy]-6-(2,6-dimethylphenyl)pyrimidin-2-yl]sulfamoyl]piperidine-3-carboxylic acid (4.2 mg, 67%) ESI-MS m z calc. 545.2672, found 546.41 (M+1)⁺; Retention time: 1.41 minutes, LC method A.

### Step 9: (11R)-11-(Cyclohexylmethyl)-6-(2,6-dimethylphenyl)-2,2-dioxo-9-oxa-2λ⁶-thia-1,3,5,12,19-pentazatricyclo[12.3.1.14,8]nonadeca-4(19),5,7-trien-13-one (Compound 73)

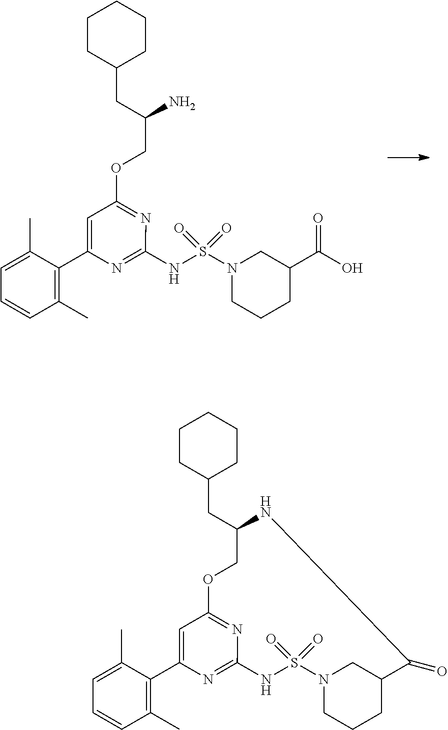 [SMILES: Cc1cccc(C)c1-c1cc(OC[C@H](N)CC2CCCCC2)nc(NS(=O)(=O)N2CCCC(C(=O)O)C2)n1 ; Cc1cccc(C)c1-c1cc2nc(n1)NS(=O)(=O)N1CCCC(C1)C(=O)N[C@H](CC1CCCCC1)CO2]

In a 4 mL vial, 1-[[4-[(2R)-2-amino-3-cyclohexyl-propoxy]-6-(2,6-dimethylphenyl)pyrimidin-2-yl]sulfamoyl]piperidine-3-carboxylic acid (4.2 mg, 0.007697 mmol) was dissolved in DMF (0.4 mL), and DIPEA (4.2 μL, 0.02411 mmol) and HATU (4 mg, 0.01052 mmol) were added. The mixture was stirred for 3 h, filtered and purified by preparative reverse phase HPLC (C₁₈): 1-99% ACN in water/HCl modifier (15 min) to yield (11R)-11-(cyclohexylmethyl)-6-(2,6-dimethylphenyl)-2,2-dioxo-9-oxa-2λ⁶-thia-1,3,5,12,19-pentazatricyclo[12.3.1.14,8]nonadeca-4(19),5,7-trien-13-one (0.8 mg, 19%) ESI-MS m/z calc. 527.25665, found 528.4 (M+1)⁺; Retention time: 1.79 minutes, LC method A.

### Example 71: Preparation of (11R)-6-(2,6-Dimethylphenyl)-11-isobutyl-2,2-dioxo-9-oxa-2λ⁶-thia-3,5,7,12-tetrazatricyclo[12.3.1.14,8]nonadeca-1(17),4,6,8(19),14(18),15-hexaen-13-one

### Step 1: Methyl 3-[(2,6-dichloropyrimidin-4-yl)sulfamoyl]benzoate

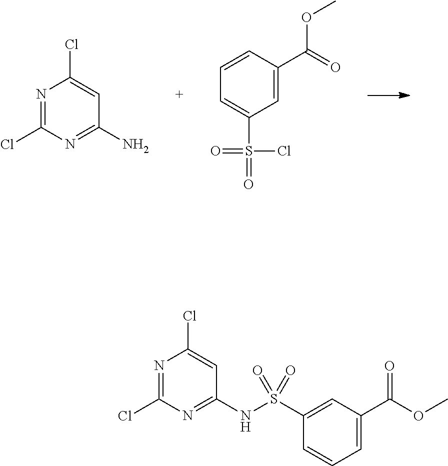 [SMILES: Nc1cc(Cl)nc(Cl)n1 ; COC(=O)c1cccc(S(=O)(=O)Cl)c1 ; COC(=O)c1cccc(S(=O)(=O)Nc2cc(Cl)nc(Cl)n2)c1]

A suspension of NaH (4.11 g, 102.8 mmol) in NMP (75 mL) was cooled to 0° C. and treated with 2,6-dichloropyrimidin-4-amine (5 g, 30.49 mmol) portionwise (6 portions) and it was stirred for 20 minutes. The reaction mixture was then treated with methyl 3-chlorosulfonylbenzoate (7.00 g, 29.83 mmol). The reaction mixture was warmed to room temperature and stirred for 2.5 h and then cooled to 0° C. and quenched with HCl (60.00 mL of 2 M, 120.0 mmol). A brown sticky oil was filtered off. The brown oil contained the product as the major component. It was dissolved in EtOAc (400 mL) and washed with water (100 mL), brine (100 mL), dried over anhydrous sodium sulfate, filtered, and concentrated in vacuo to give methyl 3-[(2,6-dichloropyrimidin-4-yl)sulfamoyl]benzoate (10.97 g, 99%) ESI-MS m/z calc. 360.9691, found 362.2 (M+1)⁺; Retention time: 0.58 minutes, LC method D.

### Step 2: 3-[(2-chloro-6-methylsulfanyl-pyrimidin-4-yl)sulfamoyl]benzoic Acid

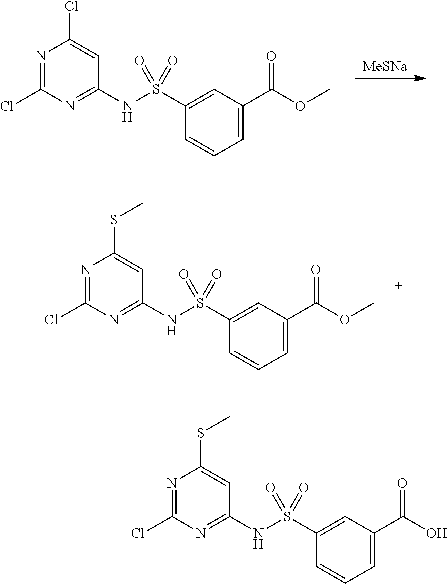 [SMILES: "MeSNa" ; COC(=O)c1cccc(S(=O)(=O)Nc2cc(Cl)nc(Cl)n2)c1 ; COC(=O)c1cccc(S(=O)(=O)Nc2cc(SC)nc(Cl)n2)c1 ; CSc1cc(NS(=O)(=O)c2cccc(C(=O)O)c2)nc(Cl)n1]

A mixture of sodium methyl sulfide (155.3 mg, 2.216 mmol) and methyl 3-[(2,6-dichloropyrimidin-4-yl)sulfamoyl]benzoate (780 mg, 2.154 mmol) in NMP (5 mL) was heated to 90° C. for 45 min. Another amount of methyl sulfide (51.2 mg, 0.7305 mmol) was added and one hour later, more methyl sulfide was added (41.5 mg, 0.5921 mmol). The reaction was stirred for an additional hour and it was cooled to room temperature. The reaction was quenched with HCl (13 mL of 1 M, 13.00 mmol) and ethyl acetate (20 mL) was added. The organic layer was separated and the aqueous layer was extracted with ethyl acetate (2×20 mL). The organic layers were combined, washed with brine (20 mL), dried over anhydrous sodium sulfate, filtered, and concentrated in vacuo. The crude residue was purified by silica chromatography (40 g) with a gradient of ethyl acetate in hexanes (0-100% over 40 minutes) to give methyl 3-[(2-chloro-6-methylsulfanyl-pyrimidin-4-yl)sulfamoyl]benzoate (38 mg, 5%) ESI-MS m/z calc. 372.9958, found 374.1 (M+1)⁺; Retention time: 0.61 minutes, and the hydrolyzed acid product 3-[(2-chloro-6-methylsulfanyl-pyrimidin-4-yl)sulfamoyl]benzoic acid (720 mg, 93%) ESI-MS m z calc. 358.98013, found 360.1 (M+1)⁺; Retention time: 0.51 minutes, LC method D.

### Step 3: 3-[[2-(2,6-Dimethylphenyl)-6-methylsulfanyl-pyrimidin-4-yl]sulfamoyl]benzoic Acid

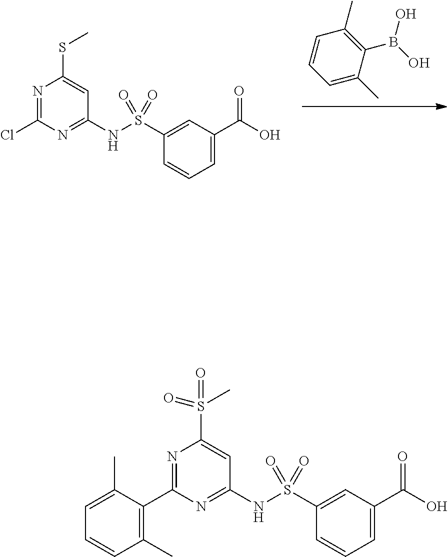 [SMILES: CSc1cc(NS(=O)(=O)c2cccc(C(=O)O)c2)nc(Cl)n1 ; Cc1cccc(C)c1B(O)O ; Cc1cccc(C)c1-c1nc(NS(=O)(=O)c2cccc(C(=O)O)c2)cc(S(C)(=O)=O)n1]

A mixture of DME (6 mL), water (1 mL), 3-[(2-chloro-6-methylsulfanyl-pyrimidin-4-yl)sulfamoyl]benzoic acid (1.0568 g, 2.937 mmol), (2,6-dimethylphenyl)boronic acid (438.8 mg, 2.926 mmol), cesium carbonate (2.8233 g, 8.665 mmol), and Pd(dppf)Cl₂ (255.2 mg, 0.3125 mmol) was microwaved at 120° C. for 20 minutes. After being cooled to room temperature, the solids were filtered. The filtrate was diluted with ethyl acetate (20 mL) and washed with 1M HCl (15 mL), water (15 mL), and brine (5 mL). The organic layer was dried over anhydrous sodium sulfate, filtered, and concentrated in vacuo to give 3-[[2-(2,6-dimethylphenyl)-6-methylsulfanyl-pyrimidin-4-yl]sulfamoyl]benzoic acid (1.26 g, 55%) ESI-MS m/z calc. 429.0817, found 430.3 (M+1)⁺; Retention time: 0.61 minutes (LC method A).

The crude product was taken up in DCM (20 mL) and cooled to 0° C. and then treated with m-CPBA (729.3 mg, 3.254 mmol). The reaction was warmed to room temperature and stirred for 3 hours and then cooled to 0° C. and quenched with sodium thiosulfate (12 mL, 75.90 mmol). The organic layer was separated and washed with a saturated solution of aqueous sodium bicarbonate (10 mL), water (10 mL) and then brine 5 mL). The organic layer was dried over anhydrous sodium sulfate, filtered, and concentrated in vacuo. The crude residue was triturated in minimal diethyl ether and the solid is collected by vacuum suction filtration. The solid wa dried under high vacuum to give 3-[[2-(2,6-dimethylphenyl)-6-methylsulfonyl-pyrimidin-4-yl]sulfamoyl]benzoic acid (720 mg, 48%) ESI-MS m/z calc. 461.07153, found 462.3 (M+1)⁺; Retention time: 0.54 minutes, LC method D.

### Step 4: (11R)-6-(2,6-Dimethylphenyl)-11-isobutyl-2,2-dioxo-9-oxa-2λ⁶-thia-3,5,7,12-tetrazatricyclo[12.3.1.14,8]nonadeca-1(17),4,6,8(19),14(18),15-hexaen-13-one

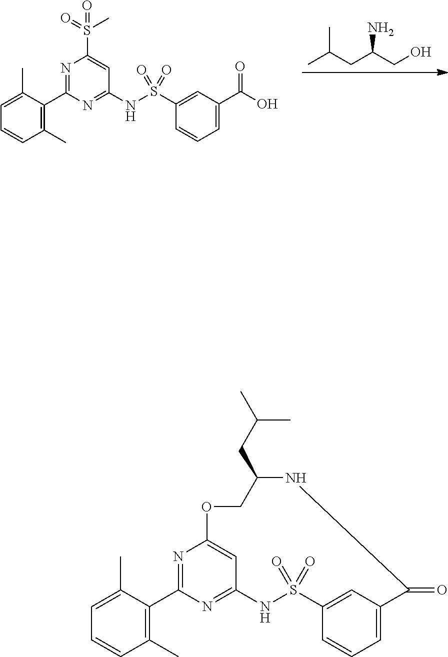

A solution of 3-[[2-(2,6-dimethylphenyl)-6-methylsulfonyl-pyrimidin-4-yl]sulfamoyl]benzoic acid (28.6 mg, 0.03408 mmol) and (2R)-2-amino-4-methyl-pentan-1-ol (5.2 mg, 0.04437 mmol) in THE (0.5 mL) was treated with sodium tert-butoxide (17.2 mg, 0.1790 mmol) and the reaction was stirred at room temperature for 1 h. The solutions were filtered and the filtrate was in 0.8 mL MeOH, and it was purified by reverse phase HPLC using a 15 min gradient of 1-99% MeCN in water (HCl modifier) to give 3-[[6-[(2R)-2-amino-4-methyl-pentoxy]-2-(2,6-dimethylphenyl)pyrimidin-4-yl]sulfamoyl]benzoic acid (6.1 mg, 36%) ESI-MS m z calc. 498.1937, found 499.5 (M+1)⁺; Retention time: 0.49 minutes, LC method A.

A solution of HATU (10 mg, 0.02630 mmol), 3-[[6-[(2R)-2-amino-4-methyl-pentoxy]-2-(2,6-dimethylphenyl)pyrimidin-4-yl]sulfamoyl]benzoic acid (6.1 mg), and TEA (10 μL, 0.07175 mmol) in DMF (1 mL) was stirred for 5 minutes at room temperature. The solutions were filtered and the filtrate diluted with 0.9 mL MeOH, and purified by reverse phase HPLC using a 15 min gradient of 1-99% MeCN in water (HCl modifier) to give (11R)-6-(2,6-dimethylphenyl)-11-isobutyl-2,2-dioxo-9-oxa-2λ⁶-thia-3,5,7,12-tetrazatricyclo[12.3.1.14,8]nonadeca-1(17),4,6,8(19),14(18),15-hexaen-13-one (1.9 mg, 32%) ESI-MS m/z calc. 480.18314, found 481.4 (M+1)⁺; Retention time: 1.45 minutes, LC method A.

### Example 72: Preparation of Compound 74

### Step 1: Methyl 3-chlorosulfonyl-5-methyl-benzoate

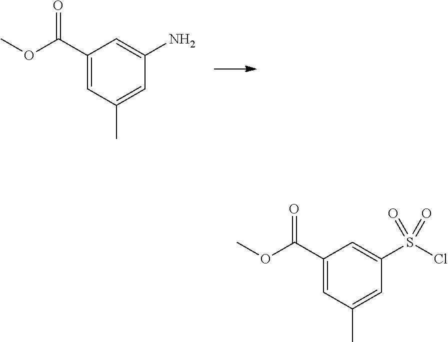

To a mixture of hydrochloric acid (12.2 mL of 37% w/v, 123.8 mmol) and acetic acid (4.1 mL, 72.10 mmol) was added methyl 3-amino-5-methyl-benzoate (3.0 g, 18.16 mmol). The flask was cooled to −10° C. (acetone and ice bath) and a solution of sodium nitrite (2.1 g, 30.44 mmol) in water (3.0 mL) was added slowly via syringe, so as to maintain the temperature (internal) below 8° C. The reaction was allowed to stir at ˜5° C. for 1.5 h. In a separate flask, sulfur dioxide (in a balloon) was bubbled through acetic acid (20.00 mL, 351.7 mmol) at 0° C. for 10 min. chlorocopper (540 mg, 5.455 mmol) was added and the bubbling continued for an additional 30 min. The solution from the first flask was added via syringe to the second flask, so as to maintain the temperature below 10° C. The bubbling of gas through the solution continued during this process. The reaction was allowed to reach room temperature and stirring continued for 1 h. Ice was added, and the stirring continued until the ice was fully melted. The solution was extracted with ether. The ether layer was washed once with a saturated solution of sodium chloride, dried over magnesium sulfate, filtered and concentrated in vacuo. The crude residue was separate by flash column chromatography on silica gel (15% ethyl acetate in hexanes) which afforded methyl 3-chlorosulfonyl-5-methyl-benzoate (2.71 g, 60%) as a yellow oil. ¹H NMR (400 MHz, Chloroform-d) δ 8.58 (d, J 2.3 Hz, 1H), 8.04 (dd, J 8.2, 2.3 Hz, 1H), 7.52 (d, J 8.2 Hz, 1H), 3.96 (s, 3H), 2.75 (s, 3H). ESI-MS m/z calc. 247.99101, found 248.98 (M+1)⁺; Retention time: 0.64 minutes, LC method D.

### Step 2: Methyl 3-[[4-chloro-6-(2,6-dimethylphenyl)pyrimidin-2-yl]sulfamoyl]-5-methyl-benzoate

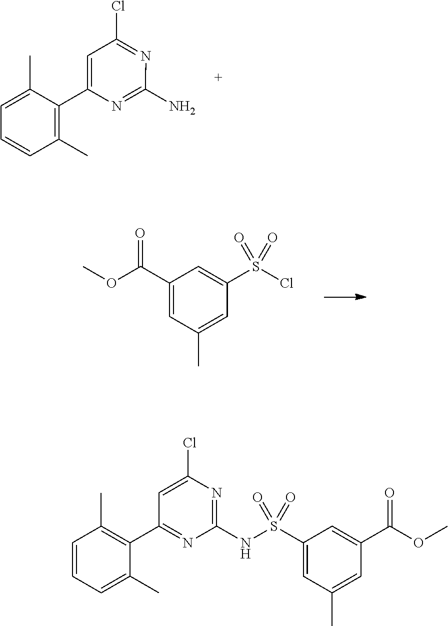 [SMILES: Cc1cccc(C)c1-c1cc(Cl)nc(N)n1 ; COC(=O)c1cc(C)cc(S(=O)(=O)Cl)c1 ; COC(=O)c1cc(C)cc(S(=O)(=O)Nc2nc(Cl)cc(-c3c(C)cccc3C)n2)c1]

To a solution of 4-chloro-6-(2,6-dimethylphenyl)pyrimidin-2-amine (600 mg, 2.567 mmol) in DMF (12.8 mL) was added sodium hydride (410 mg of 60% w/w, 10.25 mmol) at 0° C. The reaction was stirred for 15 min before adding methyl 3-chlorosulfonyl-5-methyl-benzoate (640 mg, 2.574 mmol). The reaction was further stirred for 15 min and then quenched with the addition of acetic acid (2.9 mL, 51.00 mmol). The solution was partitioned between water and ethyl acetate. The water layer was removed, and the organic layer was further washed with brine (3×), dried over magnesium sulfate, filtered and concentrated in vacuo. The crude residue was separated by flash column chromatography on silica gel (10 to 70% ethyl acetate in hexanes) which afforded methyl 3-[[4-chloro-6-(2,6-dimethylphenyl)pyrimidin-2-yl]sulfamoyl]-5-methyl-benzoate (0.980 g, 86%) as a white solid. ESI-MS m/z calc. 445.0863, found 446.14 (M+1)⁺; Retention time: 0.75 minutes, LC method D.

### Step 3: tert-Butyl N-[(1R)-1-(cyclohexylmethyl)-2-hydroxy-ethyl]carbamate

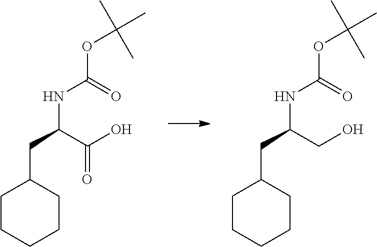 [SMILES: CC(C)(C)OC(=O)N[C@H](CC1CCCCC1)C(=O)O ; CC(C)(C)OC(=O)N[C@@H](CO)CC1CCCCC1]

A THE (400 mL) solution of (2R)-2-(tert-butoxycarbonylamino)-3-cyclohexyl-propanoic acid (50 g, 184.3 mmol) was cooled to −10° C. using a dry ice/acetone bath and Borane-Tetrahydrofuran Complex (510 mL of 1 M, 510.0 mmol) was slowly added over 20 minutes. The reaction was slowly warmed to room temperature and stirred for 2 hours and then cooled to 0° C. MeOH (360 mL, 8.887 mol) was slowly added to the reaction mixture to quench the borane reagent (delayed exotherm). The mixture was warmed to room temperature (CAUTION: gas evolution), stirred for 1 h, evaporated and co-evaporated three times with methanol to drive off all the trimethylborate and dried to give 43 g crude product. The crude was dissolved in MTBE (400 mL) and washed twice with 1M citric acid (˜2×250 mL) and twice with saturated sodium carbonate (2×250 mL) and the aqueous phases were back extracted once with MTBE (250 mL). The combined organic phases were dried, filtered and evaporated to give 38.7 g of material, which was filtered over silica gel (500 g) eluting with hexane ethyl acetate 1:1 and product fractions were evaporated and co-evaporated several times with MTBE to give tert-butyl N-[(1R)-1-(cyclohexylmethyl)-2-hydroxy-ethyl]carbamate (33 g, 63%) as a thick oil. ESI-MS m/z calc. 257.1991, found 258.0 (M+1)⁺; Retention time: 1.66 minutes, LC method A.

### Step 3: 3-[[4-Dhloro-6-(2,6-dimethylphenyl)pyrimidin-2-yl]sulfamoyl]-5-methyl-benzoic Acid

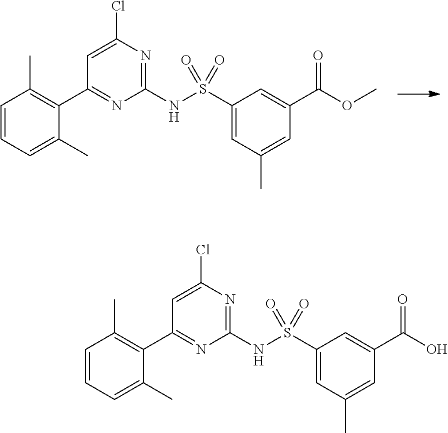 [SMILES: COC(=O)c1cc(C)cc(S(=O)(=O)Nc2nc(Cl)cc(-c3c(C)cccc3C)n2)c1 ; Cc1cc(C(=O)O)cc(S(=O)(=O)Nc2nc(Cl)cc(-c3c(C)cccc3C)n2)c1]

A biphasic solution of methyl 3-[[4-chloro-6-(2,6-dimethylphenyl)pyrimidin-2-yl]sulfamoyl]-5-methyl-benzoate (0.98 g, 2.198 mmol) and sodium hydroxide (1.75 mL of 5 M, 8.750 mmol) in THE (11 mL) and water (7.3 mL) was rapidly stirred at 60° C. in a sealed vial. The THF solvent was removed under a steady stream of air. The crude solution was acidified with hydrochloric acid (2.9 mL of 6 M, 17.40 mmol) and partitioned with ethyl acetate. The organic layer was separated, and the aqueous layer was further extracted with ethyl acetate (1×). The combined organic was dried over magnesium sulfate, filtered and concentrated in vacuo. The semi-crude solid was used without further purification. 3-[[4-chloro-6-(2,6-dimethylphenyl)pyrimidin-2-yl]sulfamoyl]-5-methyl-benzoic acid (950 mg, 94%) ESI-MS m z calc. 431.07065, found 432.17 (M+1)⁺; Retention time: 0.65 minutes, LC method D.

### Step 4: 3-[[4-[(2R)-2-Amino-3-cyclohexyl-propoxy]-6-(2,6-dimethylphenyl)pyrimidin-2-yl]sulfamoyl]-5-methyl-benzoic Acid

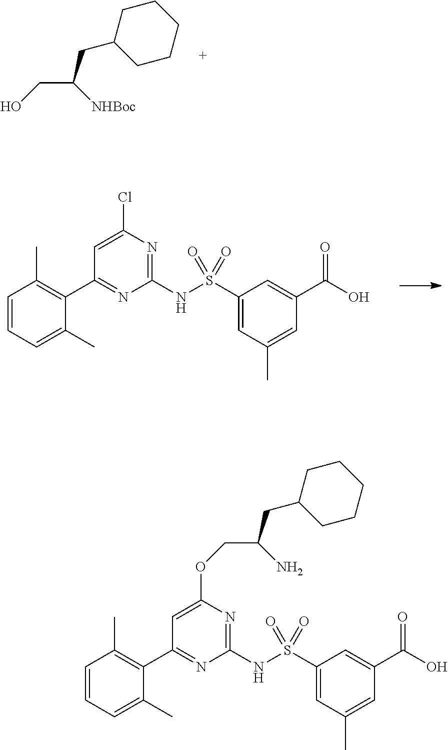 [SMILES: CC(C)(C)OC(=O)N[C@@H](CO)CC1CCCCC1 ; Cc1cc(C(=O)O)cc(S(=O)(=O)Nc2nc(Cl)cc(-c3c(C)cccc3C)n2)c1 ; Cc1cc(C(=O)O)cc(S(=O)(=O)Nc2nc(OC[C@H](N)CC3CCCCC3)cc(-c3c(C)cccc3C)n2)c1]

To a solution of tert-butyl N-[(1R)-1-(cyclohexylmethyl)-2-hydroxy-ethyl]carbamate (313 mg, 1.216 mmol) and 3-[[4-chloro-6-(2,6-dimethylphenyl)pyrimidin-2-yl]sulfamoyl]-5-methyl-benzoic acid (400.00 mg, 0.8706 mmol) in THE (4.4 mL) was added potassium tert-butoxide (489 mg, 4.358 mmol). The reaction was stirred for 3 h before acidifying with Trifluoroacetic acid (470 μL, 6.101 mmol). The solvent was removed under a steady stream of air. The residue was dissolved in DCM (4 mL) and trifluoroacetic acid (4.0 mL, 51.92 mmol) was added. The reaction was stirred for 20 min. The solvent was removed under a steady stream of air. The sample was purified by reverse phase HPLC (Waters Sunfire C₁₈ column (100×50 mm, 10 m particle size), gradient: 1-99% acetonitrile in water (5 mM HCl) over 15.0 minutes) which afforded 3-[[4-[(2R)-2-amino-3-cyclohexyl-propoxy]-6-(2,6-dimethylphenyl)pyrimidin-2-yl]sulfamoyl]-5-methyl-benzoic acid (80 mg, 17%) as a white solid. ESI-MS m/z calc. 552.24066, found 553.37 (M+1)⁺; Retention time: 0.52 minutes; LC method D.

### Step 5: (11R)-11-(Cyclohexylmethyl)-6-(2,6-dimethylphenyl)-16-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 74)

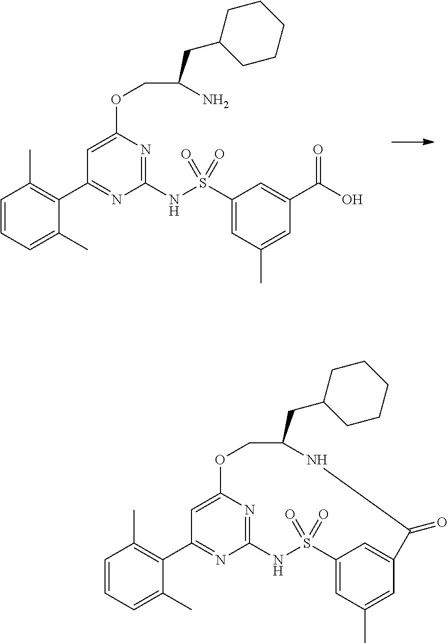 [SMILES: Cc1cc(C(=O)O)cc(S(=O)(=O)Nc2nc(OC[C@H](N)CC3CCCCC3)cc(-c3c(C)cccc3C)n2)c1 ; Cc1cc2cc(c1)S(=O)(=O)Nc1nc(cc(-c3c(C)cccc3C)n1)OC[C@@H](CC1CCCCC1)NC2=O]

To a solution of 3-[[4-[(2R)-2-amino-3-cyclohexyl-propoxy]-6-(2,6-dimethylphenyl)pyrimidin-2-yl]sulfamoyl]-5-methyl-benzoic acid (32.3 mg, 0.05844 mmol) in DMF (1.5 mL) was added HATU (27 mg, 0.07101 mmol). The reaction was stirred for 10 min before adding triethylamine (41 μL, 0.2942 mmol) and then further stirred for 15 min. The crude sample was purified by reverse phase HPLC (Phenomenex Luna C₁₈ column (75×30 mm, 5 m particle size), gradient: 1-99% acetonitrile in water (5 mM HCl) over 15.0 minutes) which afforded (11R)-11-(cyclohexylmethyl)-6-(2,6-dimethylphenyl)-16-methyl-2,2-dioxo-9-oxa-2λ⁶-thia-3,5,12,19-tetrazatricyclo[12.3.1.14,8]nonadeca-1(18),4(19),5,7,14,16-hexaen-13-one (8.7 mg, 28%) as a white solid. ESI-MS m/z calc. 534.2301, found 535.33 (M+1)⁺; Retention time: 1.8 minutes; LC method A.

### Example 73: Preparation of Compound 75

### Step 1: Methyl 3-[[4-chloro-6-(2,6-dimethylphenyl)pyrimidin-2-yl]sulfamoyl]-2-methyl-benzoate

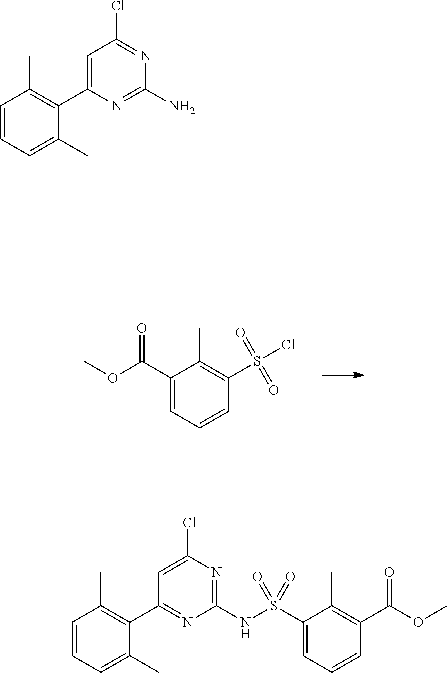 [SMILES: Cc1cccc(C)c1-c1cc(Cl)nc(N)n1 ; COC(=O)c1cccc(S(=O)(=O)Cl)c1C ; COC(=O)c1cccc(S(=O)(=O)Nc2nc(Cl)cc(-c3c(C)cccc3C)n2)c1C]

To a suspension of sodium hydride (236 mg of 60% w/w, 5.901 mmol) in DMF (5 mL) was added a solution of 4-chloro-6-(2,6-dimethylphenyl)pyrimidin-2-amine (345.3 mg, 1.478 mmol) in DMF (1.3 mL) at 0° C. The solution was stirred for 15 min before rapidly adding a solution of methyl 3-chlorosulfonyl-2-methyl-benzoate (368 mg, 1.480 mmol) in DMF (1.3 mL). The reaction was stirred for 15 min and then acidified with acetic acid (670 μL, 11.78 mmol). The solution was partitioned in between water and ethyl acetate. The aqueous layer was removed and the organic layer was further washed with brine (2×), dried over magnesium sulfate, filtered, and concentrated in vacuo. The crude residue was separate by flash column chromatography on silica gel (10 to 70% ethyl acetate in hexanes) which afforded methyl 3-[[4-chloro-6-(2,6-dimethylphenyl)pyrimidin-2-yl]sulfamoyl]-2-methyl-benzoate (440 mg, 60%) as a white solid. ESI-MS m/z calc. 445.0863, found 446.22 (M+1)⁺; Retention time: 0.74 minutes, LC method D.

### Step 2: 3-[[4-Chloro-6-(2,6-dimethylphenyl)pyrimidin-2-yl]sulfamoyl]-2-methyl-benzoic Acid

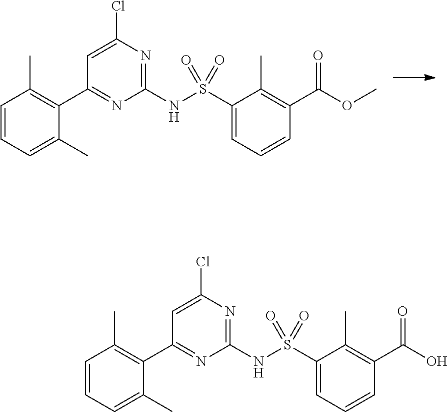 [SMILES: COC(=O)c1cccc(S(=O)(=O)Nc2nc(Cl)cc(-c3c(C)cccc3C)n2)c1C ; Cc1cccc(C)c1-c1cc(Cl)nc(NS(=O)(=O)c2cccc(C(=O)O)c2C)n1]

A solution of methyl 3-[[4-chloro-6-(2,6-dimethylphenyl)pyrimidin-2-yl]sulfamoyl]-2-methyl-benzoate (440 mg, 0.8881 mmol) and sodium hydroxide (710 μL of 5 M, 3.550 mmol) in THE (4.4 mL) and water (3 mL) was stirred rapidly for 16 h. The reaction mixture was then heated to 60° C. for 4 h. The solution was acidified with hydrochloric acid (525 μL of 37% w/v, 5.328 mmol) and the organic layer was removed under a steady stream of air from a pipette. Ethyl acetate was added, and the organic layer was then separated. The aqueous layer was further extracted with ethyl acetate (2×). The combined organics were dried over magnesium sulfate, filtered, and concentrated in vacuo. The sample was purified by reverse phase HPLC (Waters Sunfire C₁₈ column (100×50 mm, 10 m particle size), gradient: 1-99% acetonitrile in water (5 mM HCl) over 15.0 minutes) which afforded 3-[[4-chloro-6-(2,6-dimethylphenyl)pyrimidin-2-yl]sulfamoyl]-2-methyl-benzoic acid (345 mg, 85%) ESI-MS m z calc. 431.07065, found 432.17 (M+1)⁺; Retention time: 0.65 minutes as a white solid, LC method D.

### Step 3: 3-[[4-[(2R)-2-Amino-3-cyclohexyl-propoxy]-6-(2,6-dimethylphenyl)pyrimidin-2-yl]sulfamoyl]-2-methyl-benzoic Acid

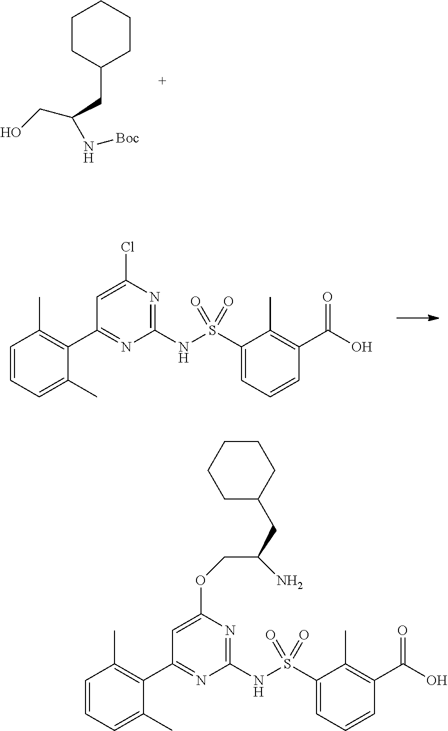

To a solution of tert-butyl N-[(1R)-1-(cyclohexylmethyl)-2-hydroxy-ethyl]carbamate (334 mg, 1.298 mmol) and 3-[[4-chloro-6-(2,6-dimethylphenyl)pyrimidin-2-yl]sulfamoyl]-2-methyl-benzoic acid (400.00 mg, 0.9262 mmol) in THE (4.6 mL) was added potassium tert-butoxide (520 mg, 4.634 mmol). The reaction was stirred for 16 h before acidifying with Trifluoroacetic acid (740 mg, 6.490 mmol). The solvent was removed under a steady stream of air from a pipette. The residue was dissolved in DMSO (0.5 mL) and acetonitrile (0.5 mL) and subsequently filtered through a syringe filter (0.45 m PTFE). The sample was purified by reverse phase HPLC (Waters Sunfire C₁₈ column (100×50 mm, 10 m particle size), gradient: 1-99% acetonitrile in water (5 mM HCl) over 30.0 minutes) which afforded 3-[[4-[(2R)-2-amino-3-cyclohexyl-propoxy]-6-(2,6-dimethylphenyl)pyrimidin-2-yl]sulfamoyl]-2-methyl-benzoic acid (16 mg, 3%) as a white solid. ESI-MS m/z calc. 552.24066, found 553.37 (M+1)⁺; Retention time: 0.53 minutes, LC method D.

### Step 4: (11R)-11-(Cyclohexylmethyl)-6-(2,6-dimethylphenyl)-18-methyl-9-oxa-2λ⁶-thia-3,5,12,19-tetraazatricyclo[12.3.1.14,8]nonadeca-1(17),4(19),5,7,14(18),15-hexaene-2,2,13-trione (Compound 75)

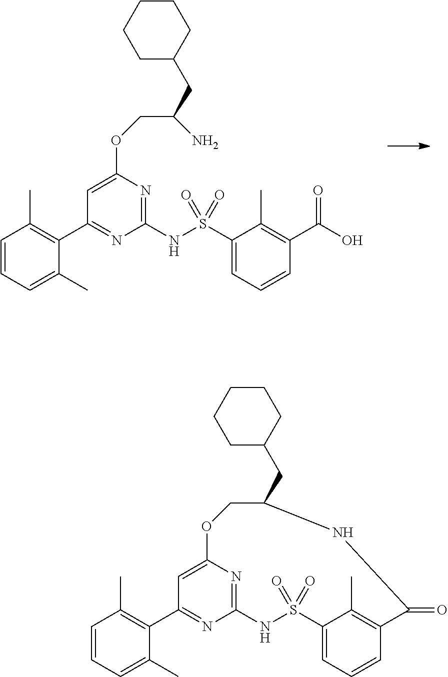 [SMILES: Cc1cccc(C)c1-c1cc(OC[C@H](N)CC2CCCCC2)nc(NS(=O)(=O)c2cccc(C(=O)O)c2C)n1 ; Cc1cccc(C)c1-c1cc2nc(n1)NS(=O)(=O)c1cccc(c1C)C(=O)N[C@H](CC1CCCCC1)CO2]

To a solution of 3-[[4-[(2R)-2-amino-3-cyclohexyl-propoxy]-6-(2,6-dimethylphenyl)pyrimidin-2-yl]sulfamoyl]-2-methyl-benzoic acid (72 mg, 0.1303 mmol) in DMF (3.25 mL) was added HATU (59 mg, 0.1552 mmol). The reaction was stirred for 10 min before adding triethylamine (90 μL, 0.6457 mmol). The reaction was stirred further for 15 min. The crude sample was purified by reverse phase HPLC (Phenomenex Luna C₁₈ column (75×30 mm, 5 m particle size), gradient: 1-99% acetonitrile in water (5 mM HCl) over 15.0 minutes) which afforded (11R)-11-(cyclohexylmethyl)-6-(2,6-dimethylphenyl)-18-methyl-9-oxa-2λ⁶-thia-3,5,12,19-tetraazatricyclo[12.3.1.14,8]nonadeca-1(17),4(19),5,7,14(18),15-hexaene-2,2,13-trione (5.5 mg, 8%) as a white solid. ESI-MS m/z calc. 534.2301, found 535.33 (M+1)⁺; Retention time: 1.78 minutes, LC method A.

### Example 74: Preparation of Compound 76

### Step 1: Ethyl 3-nitro-1H-pyrazole-5-carboxylate

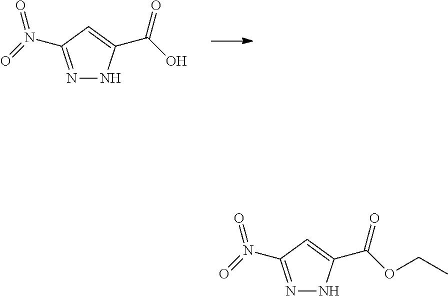

To a solution 3-nitro-1H-pyrazole-5-carboxylic acid (25 g, 159.15 mmol) in EtOH (250 mL) at rt was added acetyl chloride (37.536 g, 34 mL, 478.18 mmol) slowly. The mixture was stirred at reflux for 4 h. The mixture was concentrated and co-evaporated with EtOH (100 mL) and 1,4-dioxane (50 mL) to give ethyl 3-nitro-1H-pyrazole-5-carboxylate (30 g, 100%) as off-white solid. ESI-MS m/z calc. 185.0437, found 186.1 (M+1)⁺; Retention time: 1.58 minutes. ¹H NMR (300 MHz, CDCl₃) δ 7.41 (s, 1H), 4.47 (q, J=7.0 Hz, 2H), 1.43 (t, J=7.0 Hz, 3H), 1.25 (s, 1H), LC method K.

### Step 2: Ethyl 2-methyl-5-nitro-pyrazole-3-carboxylate

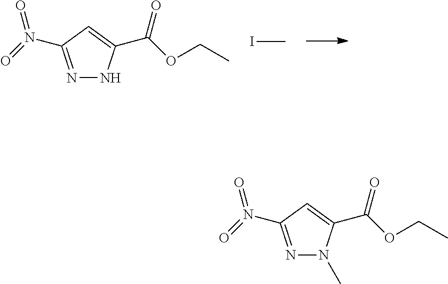

To a solution of ethyl 3-nitro-1H-pyrazole-5-carboxylate (29.6 g, 154.61 mmol) in DMF (200 mL) at 0° C. was added potassium carbonate (44.2 g, 319.81 mmol) and iodomethane (34.200 g, 15 mL, 240.95 mmol) dropwise over 15 min. The mixture was stirred at rt overnight. The mixture was cooled with ice-water bath and cold water (600 mL) was added. The precipitate was collected by filtration and washed with cold water. The resulting precipitate was dissolved in EtOAc (200 mL), dried over sodium sulfate, filtered and concentrated to dryness to give Flash ethyl 2-methyl-5-nitro-pyrazole-3-carboxylate (24.55 g, 78%) as a pale orange solid. ¹H NMR (400 MHz, CDCl₃) δ 7.41 (s, 1H), 4.42 (q, J 7.3 Hz, 2H), 4.29 (s, 3H), 1.42 (t, J 7.2 Hz, 3H). ESI-MS m/z calc. 199.0593, found 200.2 (M+1)⁺; Retention time: 1.66 minutes (LC method X).

### Step 3: Ethyl 5-amino-2-methyl-pyrazole-3-carboxylate

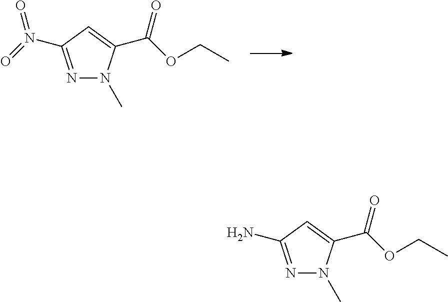

A mixture of ethyl 2-methyl-5-nitro-pyrazole-3-carboxylate (24.74 g, 124.22 mmol), 10% Palladium on carbon 50% wet (8 g, 3.7587 mmol) and MeOH (250 mL) was hydrogenated under hydrogen (balloon) for 24 h. The mixture was filtered through diatomaceous earth and washed with EtOAc. The filtrate was concentrated to give ethyl 5-amino-2-methyl-pyrazole-3-carboxylate (20.88 g, 99%) as white solid. ESI-MS m/z calc. 169.0851, found 170.1 (M+1)⁺; Retention time: 1.33 minutes. ¹H NMR (300 MHz, CDCl₃) δ 6.13 (s, 1H), 4.30 (q, J=7.1 Hz, 2H), 3.99 (s, 3H), 3.62 (br. s., 2H), 1.35 (t, J=7.0 Hz, 3H). LC method K.

### Step 4: Ethyl 5-chlorosulfonyl-2-methyl-pyrazole-3-carboxylate

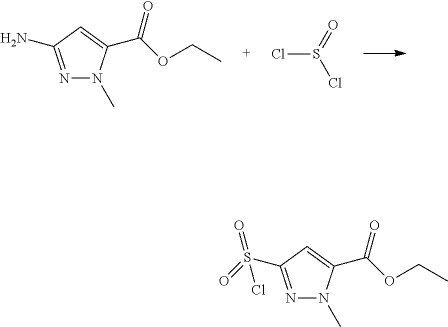

A 500-mL three-neck flask was charged with water (200 mL) and cooled with ice-water bath. Thionyl chloride (66.055 g, 40.5 mL, 555.22 mmol) was added dropwise over 20 minutes. The mixture was stirred at room temperature for 2 hours. Copper(I) chloride (800 mg, 8.0809 mmol) was added and the mixture was cooled to −5° C. Another 250-mL flask was charged with hydrochloric acid solution (37 wt %) (120 mL of 12 M, 1.4400 mol) and ethyl 5-amino-2-methyl-pyrazole-3-carboxylate (20.23 g, 107.38 mmol) was added. The mixture was cooled to −5° C. and a solution of sodium nitrite (9.26 g, 134.21 mmol) in water (50 mL) was added dropwise over 30 minutes, keeping the inner temperature between −6° C. and −3° C. The mixture was stirred at −5° C. for 30 minutes, cooled to −10° C., and slowly canulated (˜25 minutes) to the first solution. The resulting mixture was stirred at 0-5° C. (ice-water bath) for 90 minutes. More copper(I) chloride (270 mg, 2.7273 mmol) was added and the resulting mixture was stirred at 0-5° C. (ice-water bath) for 1 hour. The mixture was extracted with ethyl acetate (2×200 mL), the organic layer was dried with sodium sulfate, filtered and concentrated to dryness. The crude material was purified in two equal batches by Flash chromatography on silica gel (120 g silica gel+100 g) eluted with 0% to 20% ethyl acetate in heptane to afford ethyl 5-chlorosulfonyl-2-methyl-pyrazole-3-carboxylate (12.1 g, 43%) as a colorless oil. ¹H NMR (400 MHz, CDCl₃) δ 7.40 (s, 1H), 4.42 (q, J 7.1 Hz, 2H), 4.33 (s, 3H), 1.42 (t, J 7.1 Hz, 3H). ESI-MS m/z calc. 251.9972, found 253.0 (M+1)⁺; Retention time: 4.03 minutes (LC method Y).

### Step 5: Ethyl 5-[[4-chloro-6-(2,6-dimethylphenyl)pyrimidin-2-yl]sulfamoyl]-2-methyl-pyrazole-3-carboxylate

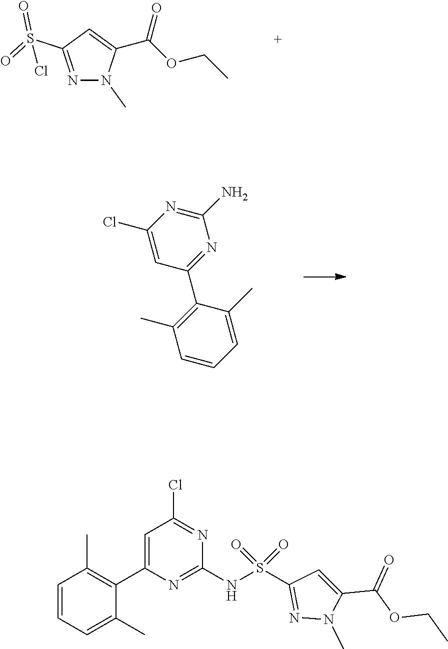 [SMILES: CCOC(=O)c1cc(S(=O)(=O)Cl)nn1C ; Cc1cccc(C)c1-c1cc(Cl)nc(N)n1 ; CCOC(=O)c1cc(S(=O)(=O)Nc2nc(Cl)cc(-c3c(C)cccc3C)n2)nn1C]

To a solution of 4-chloro-6-(2,6-dimethylphenyl)pyrimidin-2-amine (4.8 g, 20.539 mmol) in THE (140 mL) at 0° C. was added a solution of ethyl 5-chlorosulfonyl-2-methyl-pyrazole-3-carboxylate (6.13 g, 23.217 mmol), followed by sodium tert-amoxide in toluene (13.9 mL of 40% w/v, 50.486 mmol) dropwise. The mixture was stirred at rt for 1.5 h. The mixture was slowly poured into a 1 N aqueous HCl (50 mL) at 0° C. The mixture was diluted with water 100 mL and extracted with EtOAc (3×100 mL). The combined organic layers were dried over sodium sulfate filtered and concentrated to dryness. The crude material was purified by flash chromatography on silica gel (330 g) eluted with 5% to 30% ethyl acetate in heptane and the 100% ethyl acetate to give ethyl 5-[[4-chloro-6-(2,6-dimethylphenyl)pyrimidin-2-yl]sulfamoyl]-2-methyl-pyrazole-3-carboxylate (6.77 g, 72%) as white solid. ¹H NMR (400 MHz, CDCl₃) δ 7.95 (br. s., 1H), 7.49 (s, 1H), 7.23 (t, J 8.1 Hz, 1H), 7.09 (d, J 7.6 Hz, 2H), 6.94 (s, 1H), 4.36 (q, J 7.3 Hz, 2H), 4.24 (s, 3H), 2.03 (s, 6H), 1.37 (t, J 7.2 Hz, 3H). ESI-MS m/z calc. 449.0925, found 450.2 (M+1)⁺; Retention time: 4.42 minutes (LC method (LC method A).

### Step 6: 5-[[4-Chloro-6-(2,6-dimethylphenyl)pyrimidin-2-yl]sulfamoyl]-2-methyl-pyrazole-3-carboxylic Acid

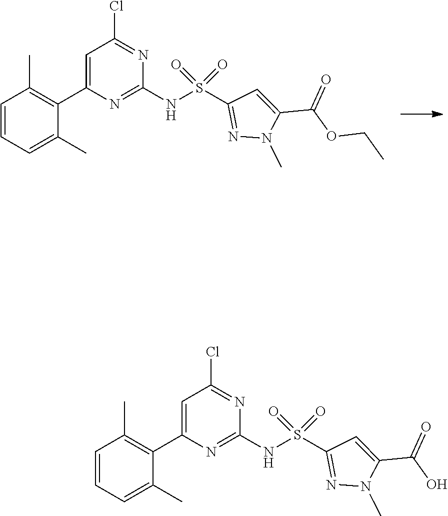 [SMILES: CCOC(=O)c1cc(S(=O)(=O)Nc2nc(Cl)cc(-c3c(C)cccc3C)n2)nn1C ; Cc1cccc(C)c1-c1cc(Cl)nc(NS(=O)(=O)c2cc(C(=O)O)n(C)n2)n1]

To a solution of ethyl 5-[[4-chloro-6-(2,6-dimethylphenyl)pyrimidin-2-yl]sulfamoyl]-2-methyl-pyrazole-3-carboxylate (7.62 g, 16.598 mmol) in THE (220 mL) at 0° C. was added a solution of NaOH (2.7 g, 67.505 mmol) in water (50 mL) and the mixture was stirred for 20 minutes. The mixture was concentrated to remove THF, diluted with water (100 mL) and washed with ethyl acetate (2×100 mL); the combined organic layers were discarded. The aqueous layer was cooled to 0° C., acidified to pH 3-4 with 1N aqueous HCl and extracted with ethyl acetate (3×150 mL). The combined organic layers were dried over sodium sulfate, filtered and concentrated to dryness to give 5-[[4-chloro-6-(2,6-dimethylphenyl)pyrimidin-2-yl]sulfamoyl]-2-methyl-pyrazole-3-carboxylic acid (7.04 g, 99%) as a white solid. ¹H NMR (400 MHz, DMSO-d₆) δ 13.83 (br. s., 1H), 12.48 (br. s., 1H), 7.33 (s, 1H), 7.24 (t, J=8.1 Hz, 1H), 7.13-7.08 (m, 3H), 4.09 (s, 3H), 1.90 (s, 6H). ESI-MS m/z calc. 421.0612, found 422.1 (M+1)⁺; Retention time: 4.04 minutes (LC method Y).

### Step 7: 5-[[4-[(2R)-2-Amino-4-methyl-pentoxy]-6-(2,6-dimethylphenyl)pyrimidin-2-yl]sulfamoyl]-2-methyl-pyrazole-3-carboxylic Acid

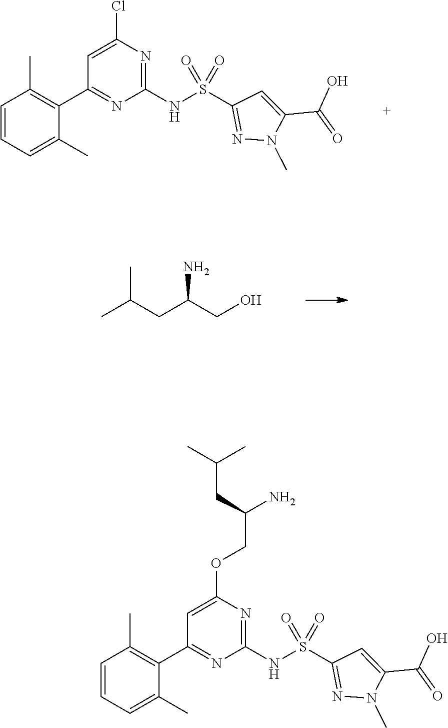

5-[[4-chloro-6-(2,6-dimethylphenyl)pyrimidin-2-yl]sulfamoyl]-2-methyl-pyrazole-3-carboxylic acid (250 mg, 0.5926 mmol) and (2R)-2-amino-4-methyl-pentan-1-ol (100 μL) were combined in THE (1.3 mL) and stirred until the reaction mixture became homogeneous. Sodium tert-butoxide (250 mg, 2.601 mmol) was added and the reaction mixture became warm to the touch and was stirred for 10 minutes without external heating. The reaction mixture was then partitioned between 1M HCl and ethyl acetate. The layers were separated and the aqueous was extracted an additional 3× with ethyl acetate. A substantial amount of product appeared to remain in the aqueous layer, so it was diluted with brine and extracted an additional 5× with ethyl acetate. The combined organics were dried over sodium sulfate and concentrated to give as an off-white solid, which was used in the next step without additional purification. 5-[[4-[(2R)-2-amino-4-methyl-pentoxy]-6-(2,6-dimethylphenyl)pyrimidin-2-yl]sulfamoyl]-2-methyl-pyrazole-3-carboxylic acid (hydrochloride salt) (317 mg, 99%) ESI-MS m/z calc. 502.19983, found 503.3 (M+1)⁺; Retention time: 0.43 minutes (LC method D).

### Step 8: (10R)-15-(2,6-Dimethylphenyl)-10-isobutyl-6-methyl-3,3-dioxo-12-oxa-3λ⁶-thia-2,5,6,9,16,17-hexazatricyclo[11.3.1.14,7]octadeca-1(17),4,7(18),13,15-pentaen-8-one (Compound 76)

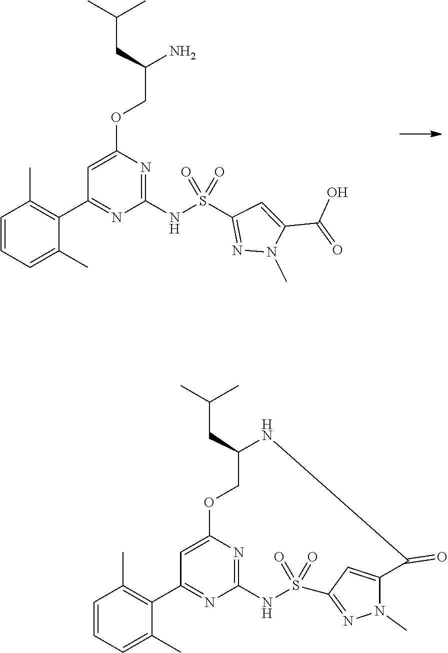 [SMILES: Cc1cccc(C)c1-c1cc(OC[C@H](N)CC(C)C)nc(NS(=O)(=O)c2cc(C(=O)O)n(C)n2)n1 ; Cc1cccc(C)c1-c1cc2nc(n1)NS(=O)(=O)c1cc(n(C)n1)C(=O)N[C@H](CC(C)C)CO2]

A solution of 5-[[4-[(2R)-2-amino-4-methyl-pentoxy]-6-(2,6-dimethylphenyl)pyrimidin-2-yl]sulfamoyl]-2-methyl-pyrazole-3-carboxylic acid (hydrochloride salt) (25 mg, 0.04638 mmol) in DMF (5 mL) was added several drops at a time over the course of an hour to a stirring solution of HATU (35 mg, 0.09205 mmol) and DIPEA (50 μL, 0.2871 mmol) in DMF (10 mL). The reaction mixture was allowed to stir at room temperature for one hour after addition was completed. The reaction mixture was then partitioned between 1M HCl and ethyl acetate and the aqueous layer was extracted an additional 3× with ethyl acetate. The combined organics were washed with brine, dried over sodium sulfate, and concentrated. The resulting crude material was purified by prep HPLC (1-70% ACN in water, HCl modifier, 15 minute run) to give (10R)-15-(2,6-dimethylphenyl)-10-isobutyl-6-methyl-3,3-dioxo-12-oxa-3λ⁶-thia-2,5,6,9,16,17-hexazatricyclo[11.3.1.14,7]octadeca-1(17),4,7(18),13,15-pentaen-8-one (7.6 mg, 33%), ESI-MS m/z calc. 484.18927, found 485.3 (M+1)⁺; Retention time: 1.37 minutes (LC method A).

### Example 75: Preparation of Compound 77

### Step 1: Methyl 5-[(4-methoxyphenyl)methylsulfanyl]-1-methyl-pyrazole-3-carboxylate

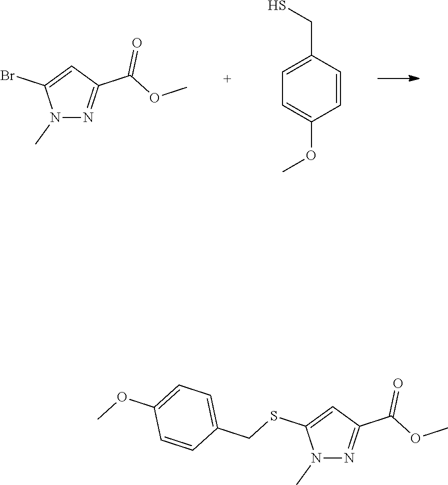

To a sealed tube was added methyl 5-bromo-1-methyl-pyrazole-3-carboxylate (4.71 g, 21.503 mmol), (4-methoxyphenyl)methanethiol (3.32 g, 21.526 mmol) and diisopropylethylamine (5.5650 g, 7.5 mL, 43.058 mmol) in dioxane (100 mL). The mixture was sparged with nitrogen gas for 15 minutes, then added Xantphos (1.24 g, 2.1430 mmol) and Pd2dba3 (980 mg, 1.0702 mmol). The tube was capped and heated in an oil bath set at 100° C. for 5 hours. Once cooled to room temperature, the reaction mixture was transferred to a 1.0-L separatory funnel with water (350 mL) and the aqueous layer was extracted with ethyl acetate (1×300 mL, 1×200 mL). The combined organic layers were washed with brine, dried over sodium sulfate, filtered and concentrated under reduced pressure. The residue was purified by silica gel chromatography on a 220-g column, eluting from 0% to 40% ethyl acetate in heptanes, to afford methyl 5-[(4-methoxyphenyl)methylsulfanyl]-1-methyl-pyrazole-3-carboxylate (5.2 g, 83%) as a pale-yellow solid. ESI-MS m/z calc. 292.0882, found 293.1 (M+1)⁺; Retention time: 1.94 minutes, LC method K.

### Step 2: Methyl 5-chlorosulfonyl-1-methyl-pyrazole-3-carboxylate

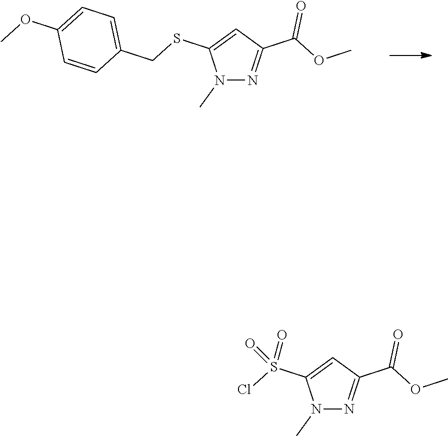

A solution of methyl 5-[(4-methoxyphenyl)methylsulfanyl]-1-methyl-pyrazole-3-carboxylate (4.74 g, 16.213 mmol) in acetic acid (50 mL) and water (25 mL) was treated with N-chlorosuccinimide (6.6 g, 49.426 mmol) at room temperature for 1.5 hours. The reaction was then quenched by adding to a 2.0-L separatory funnel containing cold water (1.5 L) and the aqueous layer was extracted with MTBE (3×250 mL). The combined organic layers were washed with cold water (300 mL), brine, dried over sodium sulfate, filtered and concentrated under reduced pressure. The residue was purified by silica gel chromatography on a 220-g column eluting from 0% to 40% ethyl acetate in heptanes to afford methyl 5-chlorosulfonyl-1-methyl-pyrazole-3-carboxylate (3.62 g, 90%) as a colorless oil. ¹H NMR. ¹H NMR (300 MHz, CDCl₃) δ 7.50 (s, 1H), 4.30 (s, 3H), 3.96 (s, 3H). ESI-MS m/z calc. 237.9815, found 239.0 (M+1)⁺; Retention time: 1.81 minutes, LC method K.

### Step 3: Methyl 5-[[4-chloro-6-(2,6-dimethylphenyl)pyrimidin-2-yl]sulfamoyl]-1-methyl-pyrazole-3-carboxylate

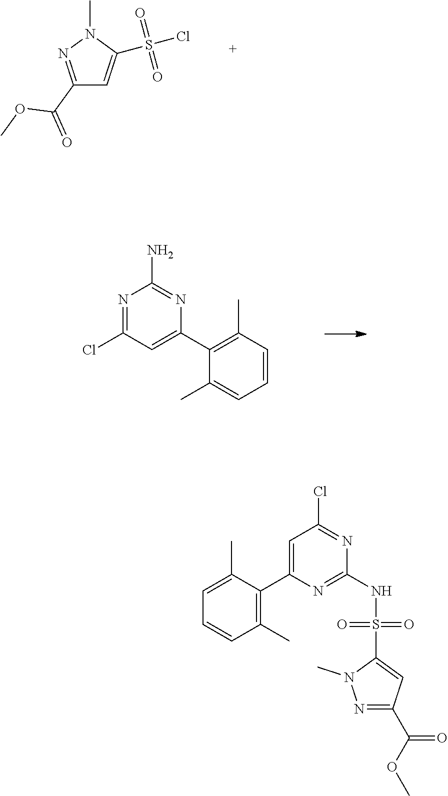 [SMILES: COC(=O)c1cc(S(=O)(=O)Cl)n(C)n1 ; Cc1cccc(C)c1-c1cc(Cl)nc(N)n1 ; COC(=O)c1cc(S(=O)(=O)Nc2nc(Cl)cc(-c3c(C)cccc3C)n2)n(C)n1]

4-chloro-6-(2,6-dimethylphenyl)pyrimidin-2-amine (7.65 g, 32.735 mmol) was dissolved in THE (140 mL) and cooled in an ice bath under stirring and nitrogen. To the cold solution, methyl 5-chlorosulfonyl-1-methyl-pyrazole-3-carboxylate (6.24 g, 26.147 mmol) in solution in THE (45 mL) was added. At 0° C., 2-methyl-butan-2-ol(Sodium Ion (1)) (18.5 mL of 40% w/v, 66.584 mmol) was added dropwise (the color was colorless before and yellow after the addition) and the reaction was stirred at room temperature for two hour. The reaction was quenched with HCl 1 N (50 mL). The reaction was diluted with water (150 mL) and EtOAc (250 mL). The organic phase was isolated, and the aqueous phase was extracted with EtOAc (200 mL). The organic phases were combined and washed with water (100 mL and brine (100 mL). The organic phase was dried over sodium sulfate, filtered, and concentrated. The crude was purified by chromatography on silica gel, 120 g, eluted with EtOAc-heptane 5% to 35% to give methyl 5-[[4-chloro-6-(2,6-dimethylphenyl)pyrimidin-2-yl]sulfamoyl]-1-methyl-pyrazole-3-carboxylate (9.15 g, 80%) as a beige solid. ESI-MS m/z calc. 435.0768, found 436.1 (M+1)⁺; Retention time: 1.98 minutes, LC method K.

### Step 4: 5-[[4-Chloro-6-(2,6-dimethylphenyl)pyrimidin-2-yl]sulfamoyl]-1-methyl-pyrazole-3-carboxylic Acid

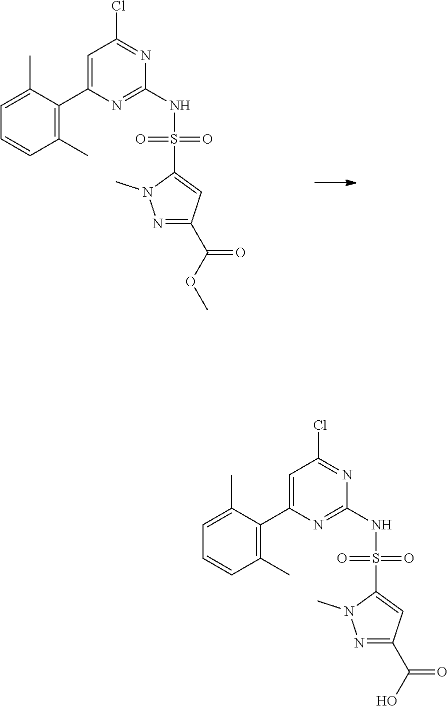 [SMILES: COC(=O)c1cc(S(=O)(=O)Nc2nc(Cl)cc(-c3c(C)cccc3C)n2)n(C)n1 ; Cc1cccc(C)c1-c1cc(Cl)nc(NS(=O)(=O)c2cc(C(=O)O)nn2C)n1]

A mixture of methyl 5-[[4-chloro-6-(2,6-dimethylphenyl)pyrimidin-2-yl]sulfamoyl]-1-methyl-pyrazole-3-carboxylate (832 mg, 1.9088 mmol) in THE (25 mL) and water (25 mL) was treated with lithium hydroxide hydrate (240 mg, 5.7192 mmol) and stirred vigorously at room temperature for 2.5 hours. Most of the THF was removed under reduced pressure, and the remaining aqueous layer was transferred to a 250-mL separatory funnel with water (100 mL) and the aqueous layer was washed with DCM (50 mL). The aqueous layer was acidified to a pH of about 4 using solid citric acid and extracted with ethyl acetate (3×50 mL). The combined organic layers were washed with water (50 mL), brine (50 mL), dried over sodium sulfate, filtered and concentrated under reduced pressure to afford 5-[[4-chloro-6-(2,6-dimethylphenyl)pyrimidin-2-yl]sulfamoyl]-1-methyl-pyrazole-3-carboxylic acid (719 mg, 86%) as a white solid. ¹H NMR (300 MHz, DMSO-d₆) δ 13.14 (br. s., 2H), 7.37 (s, 1H), 7.31-7.22 (m, 1H), 7.18-7.08 (m, 3H), 3.99 (s, 3H), 1.93 (s, 6H). ESI-MS m/z calc. 421.0612, found 422.1 (M+1)⁺; Retention time: 2.62 minutes, LC method U.

### Step 5: 5-[[4-[(2R)-2-Amino-4,4-dimethyl-pentoxy]-6-(2,6-dimethylphenyl)pyrimidin-2-yl]sulfamoyl]-1-methyl-pyrazole-3-carboxylic Acid

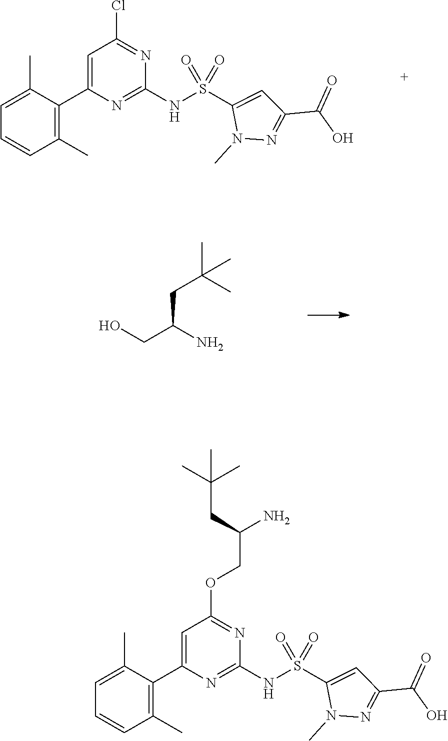

5-[[4-Chloro-6-(2,6-dimethylphenyl)pyrimidin-2-yl]sulfamoyl]-1-methyl-pyrazole-3-carboxylic acid (1.50 g, 3.556 mmol) and (2R)-2-amino-4,4-dimethyl-pentan-1-ol (hydrochloride salt) (656 mg, 3.912 mmol) were combined and dissolved/suspended in THE (12 mL). Solid sodium tert-butoxide (1.71 g, 17.79 mmol) was added in gradual portions over 2 minutes. The reaction mixture was allowed to stir at room temperature for 2 hours. The reaction was quenched with the addition of aqueous HCl (75 mL, 1 M). It was then extracted with EtOAc (3×75 mL). The organic layers were combined, washed with brine (1×100 mL), dried over sodium sulfate, filtered and concentrated under reduced pressure. The crude product was chromatographed on a 24-gram silica gel column eluting with a 0-20% MeOH/DCM gradient over 40 minutes; product eluted at 10% MeOH. The obtained white solid was dissolved into MeOH/DCM, and HCl (800 μL of 4 M, 3.200 mmol) in dioxane was added. After brief stirring, volatiles were removed under reduced pressure to provide 5-[[4-[(2R)-2-amino-4,4-dimethyl-pentoxy]-6-(2,6-dimethylphenyl)pyrimidin-2-yl]sulfamoyl]-1-methyl-pyrazole-3-carboxylic acid (hydrochloride salt) (1.112 g, 57%) was obtained as a pinkish-white solid. ¹H NMR (400 MHz, DMSO-d₆) δ 8.15 (s, 2H), 7.32 (t, J 7.6 Hz, 1H), 7.19 (s, 1H), 7.17 (s, 1H), 7.12 (s, 1H), 6.33 (s, 1H), 4.31 (dd, J 11.9, 3.1 Hz, 1H), 4.13 (d, J 4.1 Hz, 1H), 4.03 (s, 3H), 3.57 (s, 1H), 2.13 (s, 6H), 1.63-1.47 (m, 2H), 0.95 (s, 9H). ESI-MS m/z calc. 516.2155, found 517.2 (M+1)⁺; Retention time: 1.16 minutes (LC method A).

### Step 6: (10R)-15-(2,6-Dimethylphenyl)-10-(2,2-dimethylpropyl)-5-methyl-3,3-dioxo-12-oxa-3λ⁶-thia-2,5,6,9,16,17-hexazatricyclo[11.3.1.14,7]octadeca-1(17),4(18),6,13,15-pentaen-8-one (Compound 77)

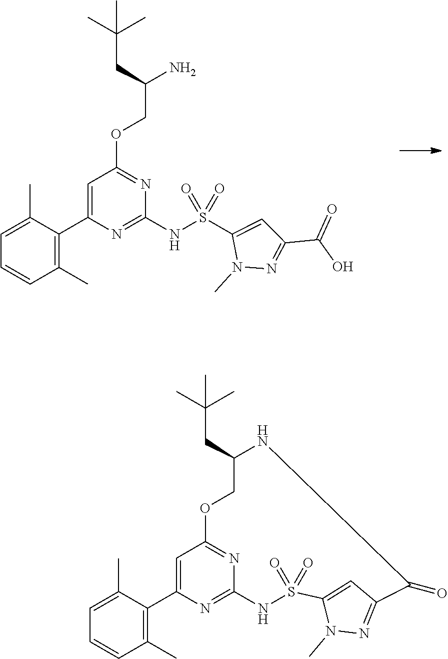 [SMILES: Cc1cccc(C)c1-c1cc(OC[C@H](N)CC(C)(C)C)nc(NS(=O)(=O)c2cc(C(=O)O)nn2C)n1 ; Cc1cccc(C)c1-c1cc2nc(n1)NS(=O)(=O)c1cc(nn1C)C(=O)N[C@H](CC(C)(C)C)CO2]

5-[[4-[(2R)-2-Amino-4,4-dimethyl-pentoxy]-6-(2,6-dimethylphenyl)pyrimidin-2-yl]sulfamoyl]-1-methyl-pyrazole-3-carboxylic acid (Hydrochloride salt) (50 mg, 0.09040 mmol) was dissolved in DMF (1 mL). HATU (41 mg, 0.1078 mmol) was added followed by DIEA (100 μL, 0.5741 mmol). The reaction mixture was allowed to stir at room temperature overnight. The reaction mixture was concentrated and re-dissolved in DMF (1.5 mL). It was filtered, and the product was isolated by mass-triggered reverse-phase HPLC: Samples were purified using a reverse phase HPLC-MS method using a Luna C₁₈ (2) column (75×30 mm, 5 μm particle size) sold by Phenomenex (pn: 00C-4252-U0-AX), and a dual gradient run from 1-99% mobile phase B over 15.0 minutes. Mobile phase A=water (5 mM HCl). Mobile phase B=acetonitrile. Flow rate=50 mL/min, injection volume=950 μL, and column temperature=25° C. (10R)-15-(2,6-dimethylphenyl)-10-(2,2-dimethylpropyl)-5-methyl-3,3-dioxo-12-oxa-3λ⁶-thia-2,5,6,9,16,17-hexazatricyclo[11.3.1.14,7]octadeca-1(17),4(18),6,13,15-pentaen-8-one (2.8 mg, 6%) was obtained. ¹H NMR (400 MHz, DMSO-d₆) δ 13.28 (s, 1H), 7.83 (d, J 8.2 Hz, 1H), 7.29 (t, J 7.6 Hz, 1H), 7.19 (s, 1H), 7.15 (d, J 7.6 Hz, 2H), 6.38 (s, 1H), 4.75 (dd, J 10.5, 4.5 Hz, 1H), 3.93 (s, 3H), 3.54 (t, J 11.0 Hz, 1H), 2.75 (d, J 7.9 Hz, 1H), 2.05 (d, J 47.2 Hz, 6H), 1.49 (dd, J 14.6, 8.9 Hz, 1H), 1.34 (d, J 14.3 Hz, 1H), 0.73 (s, 9H).

The product was purified a second time to give (10R)-15-(2,6-dimethylphenyl)-10-(2,2-dimethylpropyl)-5-methyl-3,3-dioxo-12-oxa-3λ⁶-thia-2,5,6,9,16,17-hexazatricyclo[11.3.1.14,7]octadeca-1(17),4(18),6,13,15-pentaen-8-one (2.8 mg). ESI-MS m/z calc. 498.20493, found 499.14 (M+1)⁺, Retention time: 1.59 minutes (LC method A).

### Example 76: Preparation of Compound 78

### Step 1: Ethyl 5-nitro-1-(2-trimethylsilylethoxymethyl)pyrazole-3-carboxylate and ethyl 5-nitro-2-(2-trimethylsilylethoxymethyl)pyrazole-3-carboxylate

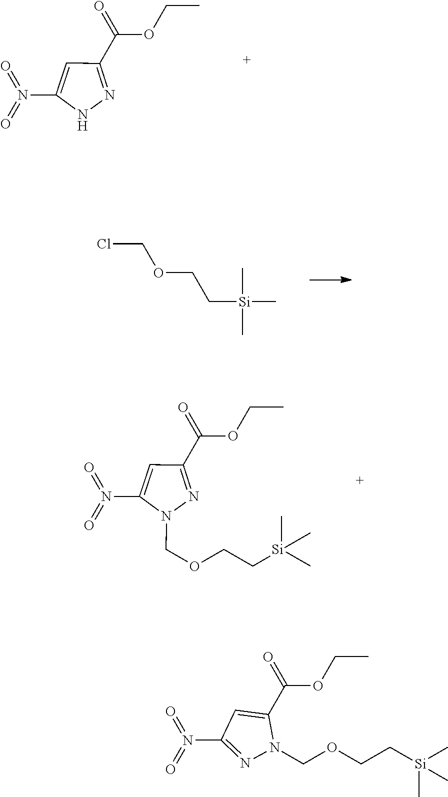

In a 500-mL round-bottomed flask, ethyl 5-nitro-1H-pyrazole-3-carboxylate (13.8990 g, 72.82 mmol) was dissolved in THF (200 mL) and cooled to 0° C. NaH (2.5227 g of 95% w/w, 99.87 mmol) was added portionwise under nitrogen gas (CAUTION: GAS EVOLUTION). Then, SEM-Cl (15.0 mL, 84.75 mmol) was added in one portion. The resulting mixture was stirred at 0° C. for 30 min, and then warmed to room temperature over 24 h. After this time, a second portion of NaH (2.5227 g of 95% w/w, 99.87 mmol) and SEM-Cl (15.0 mL, 84.75 mmol) were added, and stirred for 22 h. This mixture was then quenched by pouring into 1 N HCl solution (300 mL), and extracted with ethyl acetate (3×250 mL). The combined organic extracts was washed with water (400 mL) and saturated aqueous sodium chloride solution (400 mL), then dried over sodium sulfate, filtered, and evaporated in vacuo to give a brown liquid. Purification by silica gel chromatography (330 g of silica) using a gradient eluent of 1 to 30% ethyl acetate in hexanes gave two batches of product: batch 1, 5.367 g of a slightly yellow liquid, impure product but only one regioisomer; and batch 2, 10.915 g of a slightly yellow liquid, pure product as a mixture of regioisomers. Combined, they give: ethyl 5-nitro-1-(2-trimethylsilylethoxymethyl)pyrazole-3-carboxylate; ethyl 5-nitro-2-(2-trimethylsilylethoxymethyl)pyrazole-3-carboxylate (16.282 g, 71%). For the first regioisomer: ¹H NMR (499 MHz, Chloroform-d) δ 7.45 (s, 1H), 5.91 (s, 2H), 4.42 (q, J 7.1 Hz, 2H), 3.65 (t, J 7.9 Hz, 2H), 1.41 (t, J 7.1 Hz, 3H), 0.91 (t, J 7.9 Hz, 2H),−0.04 (s, 9H); and for the second of the two regioisomers:¹H NMR (499 MHz, Chloroform-d) δ 7.56 (s, 1H), 5.95 (s, 2H), 4.44 (q, J 6.8 Hz, 2H), 3.62 (t, J 8.1 Hz, 2H), 1.41 (t, J 7.1 Hz, 3H), 0.89 (t, J 7.9 Hz, 2H),−0.04 (s, 9H).

### Step 2: Ethyl 5-amino-1-(2-trimethylsilylethoxymethyl)pyrazole-3-carboxylate; ethyl 5-amino-2-(2-trimethylsilylethoxymethyl)pyrazole-3-carboxylate

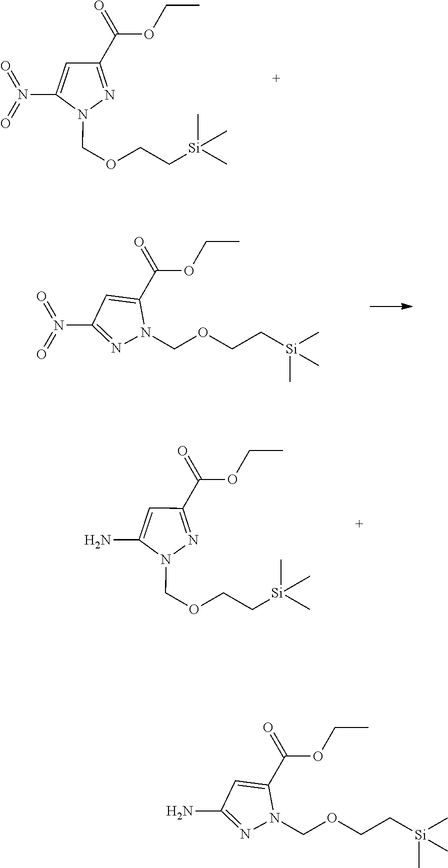 [SMILES: CCOC(=O)c1cc([N+](=O)[O-])n(COCC[Si](C)(C)C)n1 ; CCOC(=O)c1cc([N+](=O)[O-])nn1COCC[Si](C)(C)C ; CCOC(=O)c1cc(N)n(COCC[Si](C)(C)C)n1 ; CCOC(=O)c1cc(N)nn1COCC[Si](C)(C)C]

The mixture of products from step 1, ethyl 5-nitro-1-(2-trimethylsilylethoxymethyl)pyrazole-3-carboxylate; ethyl 5-nitro-2-(2-trimethylsilylethoxymethyl)pyrazole-3-carboxylate (16.282 g, 51.62 mmol), was used as two separate batches: “Batch 1” (5.367 g) and “Batch 2” (10.915 g). A total of Pd(OH)₂/C (3.75 g of 10% w/w, 2.670 mmol) and EtOH (75 mL), as well as balloons of hydrogen (2 L, 79.37 mmol) were used in this experiment.

“Batch 1”:In a 100-mL round-bottomed flask, “Batch 1” (5.367 g) was dissolved in EtOH (25 mL), and this solution was purged with a balloon of nitrogen gas. The cap was briefly removed, and Pd(OH)₂/C (1.25 g of 10% w/w, 0.890 mmol) was added. This reaction mixture was stirred under hydrogen gas (2 L, 79.37 mmol) at room temperature for 16.5 h, then at 50° C. for 23 h, then at 70° C. for 4 days. It was then filtered through Celite and rinsed with ethanol (75 mL). The organic solution was evaporated in vacuo. The resulting brown oil was purified by silica gel chromatography (120 g of silica) using a gradient eluent of 1 to 10% methanol in dichloromethane to give an orange oil (3.279 g).

“Batch 2”:In a 250-mL round-bottomed flask, “Batch 2” (10.915 g) was dissolved in EtOH (50 mL), and this solution was purged with a balloon of nitrogen gas. The cap was briefly removed, and Pd(OH)₂/C (2.50 g of 10% w/w, 1.780 mmol) was added. This reaction mixture was stirred under hydrogen gas (2 L, 79.37 mmol) at room temperature for 16.5 h, then at 50° C. for 23 h. It was then filtered through Celite and rinsed with ethanol (150 mL). The organic solution was evaporated in vacuo. The resulting brown oil was purified by silica gel chromatography (330 g of silica) using a gradient eluent of 1 to 10% methanol in dichloromethane to give a yellow oil that solidifies under high vacuum (7.01 g).

Combined, they give: ethyl 5-amino-1-(2-trimethylsilylethoxymethyl)pyrazole-3-carboxylate; ethyl 5-amino-2-(2-trimethylsilylethoxymethyl)pyrazole-3-carboxylate (10.289 g, 70%) ESI-MS m/z calc. 285.15088, found 286.2 (M+1)⁺; Retention time: 1.49 minutes and 1.57 minutes (LC method A). For the first of the two regioisomers (36% of sample):¹H NMR (499 MHz, dimethylsulfoxide-d₆) δ 6.08 (s, 1H), 5.48 (s, 2H), 5.00 (s, 2H), 4.25 (q, J 7.1 Hz, 2H), 3.49 (t, J 7.9 Hz, 2H), 1.27 (t, J 7.1 Hz, 3H), 0.78 (t, J 7.3 Hz, 2H),−0.07 (s, 9H); and for the second of the two regioisomers (64% of sample):¹H NMR (499 MHz, dimethylsulfoxide-d₆) δ 5.68 (s, 1H), 5.55 (s, 2H), 5.29 (s, 2H), 4.20 (q, J 7.1 Hz, 2H), 3.54 (t, J 7.9 Hz, 2H), 1.25 (t, J 7.1 Hz, 3H), 0.83 (t, J 7.9 Hz, 2H),−0.04 (s, 9H).

### Step 3: Ethyl 5-chlorosulfonyl-1-(2-trimethylsilylethoxymethyl)pyrazole-3-carboxylate and ethyl 5-chlorosulfonyl-2-(2-trimethylsilylethoxymethyl)pyrazole-3-carboxylate

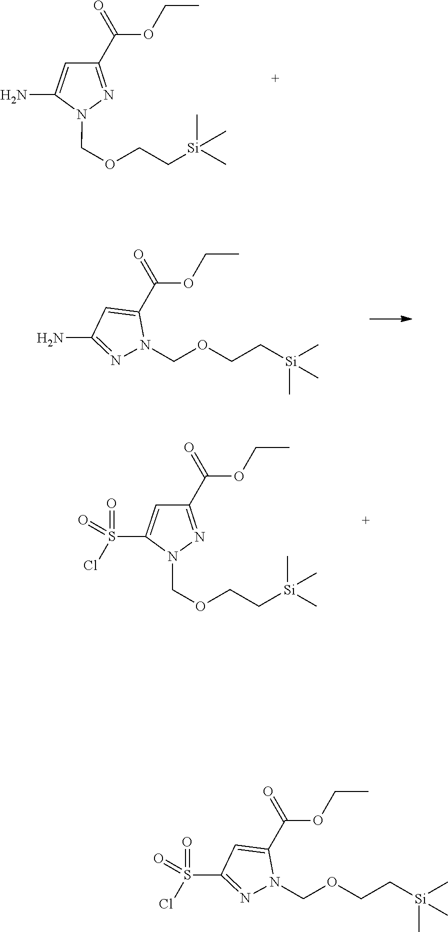 [SMILES: CCOC(=O)c1cc(N)n(COCC[Si](C)(C)C)n1 ; CCOC(=O)c1cc(N)nn1COCC[Si](C)(C)C ; CCOC(=O)c1cc(S(=O)(=O)Cl)n(COCC[Si](C)(C)C)n1 ; CCOC(=O)c1cc(S(=O)(=O)Cl)nn1COCC[Si](C)(C)C]

In a 100-mL round-bottomed flask, a solution of ethyl 5-amino-1-(2-trimethylsilylethoxymethyl)pyrazole-3-carboxylate; ethyl 5-amino-2-(2-trimethylsilylethoxymethyl)pyrazole-3-carboxylate (3.2790 g, 11.49 mmol) in acetic acid (27 mL): water (14 mL): HCl (14 mL of 12.1 M, 169.4 mmol) was prepared and cooled to −10° C. (dry ice in 1:1 brine:water). A solution of NaNO₂ (0.8652 mg, 0.01254 mmol) in water (9 mL) was added, and this mixture was stirred open to air at −10° C. for 30 min. In a separate 200-mL round-bottomed flask, a suspension of CuSO₄ (0.4261 g, 2.670 mmol) in acetic acid (40 mL) was prepared and saturated with SO₂ (6 g, 93.66 mmol) using a balloon full of SO₂ gas. This suspension was cooled to −10° C. The substrate mixture prepared above was added onto this CuSO₄ suspension, and the resulting mixture was stirred at −10° C. for 1 h. After this time, it was allowed to warm to room temperature and quenched with water (500 mL). The pH of the mixture was adjusted to 7 with 1 N NaOH solution, and the mixture was extracted with ethyl acetate (3×400 mL). The combined organic extracts was washed with water (400 mL) and saturated aqueous sodium chloride solution (400 mL), then dried over sodium sulfate, filtered, and evaporated in vacuo to give a brown oil, ethyl 5-chlorosulfonyl-1-(2-trimethylsilylethoxymethyl)pyrazole-3-carboxylate; ethyl 5-chlorosulfonyl-2-(2-trimethylsilylethoxymethyl)pyrazole-3-carboxylate (2.882 g, 68%) ¹H NMR (500 MHz, chloroform-d) δ 7.43 (s, 1H), 5.95 (s, 2H), 4.42 (q, J 7.1 Hz, 2H), 3.69-3.61 (m, 2H), 1.40 (t, J=7.1 Hz, 3H), 0.93-0.86 (m, 2H),−0.04 (s, 9H).

### Step 4: Ethyl 5-[[4-chloro-6-(2,6-dimethylphenyl)pyrimidin-2-yl]sulfamoyl]-1-(2-trimethylsilylethoxymethyl)pyrazole-3-carboxylate and ethyl 5-[[4-chloro-6-(2,6-dimethylphenyl)pyrimidin-2-yl]sulfamoyl]-2-(2-trimethylsilylethoxymethyl)pyrazole-3-carboxylate

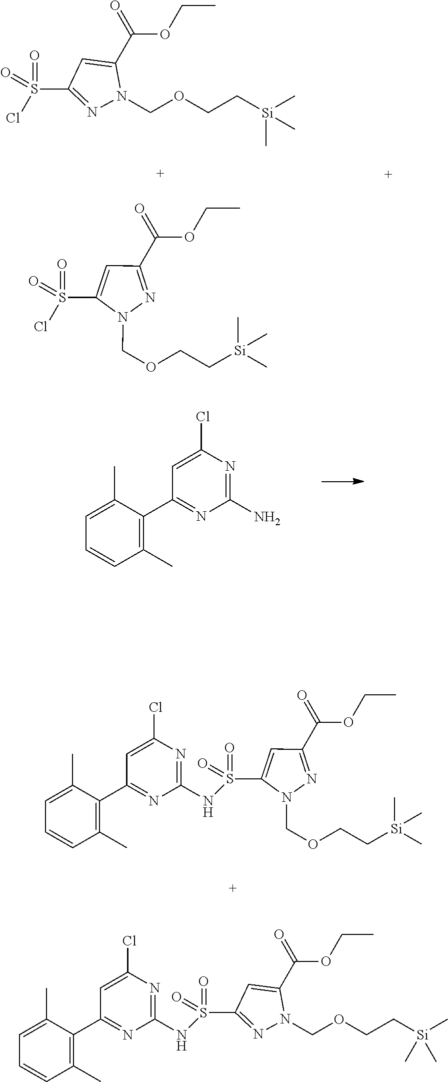 [SMILES: CCOC(=O)c1cc(S(=O)(=O)Cl)nn1COCC[Si](C)(C)C ; CCOC(=O)c1cc(S(=O)(=O)Cl)n(COCC[Si](C)(C)C)n1 ; Cc1cccc(C)c1-c1cc(Cl)nc(N)n1 ; CCOC(=O)c1cc(S(=O)(=O)Nc2nc(Cl)cc(-c3c(C)cccc3C)n2)n(COCC[Si](C)(C)C)n1 ; CCOC(=O)c1cc(S(=O)(=O)Nc2nc(Cl)cc(-c3c(C)cccc3C)n2)nn1COCC[Si](C)(C)C]

Three attempts (with differing amounts of substrate) were made in this experiment.

Try 1: In a 3-mL vial, 4-chloro-6-(2,6-dimethylphenyl)pyrimidin-2-amine (50.5 mg, 0.2161 mmol) was dissolved in DMF (500 μL), to which ethyl 5-chlorosulfonyl-1-(2-trimethylsilylethoxymethyl)pyrazole-3-carboxylate; ethyl 5-chlorosulfonyl-2-(2-trimethylsilylethoxymethyl)pyrazole-3-carboxylate (159.5 mg, 0.4324 mmol) and NaOtBu (45.2 mg, 0.4703 mmol) were added. The resulting mixture was stirred at 80° C. for 1 h, then at 120° C. for 1 h. It was cooled to room temperature, then 1 N HCl solution (500 μL) was added, followed by EtOAc (800 μL). The phases were vigorously mixed and then allowed to settle into two layers. The organic layer was filtered and purified by reverse phase preparative chromatography using a C₁₈ column and a gradient eluent of 1 to 99% acetonitrile in water containing 5 mM hydrochloric acid to give 4.0 mg of product.

Try 2: In a 20-mL vial, 4-chloro-6-(2,6-dimethylphenyl)pyrimidin-2-amine (252.4 mg, 1.080 mmol) was dissolved in DMF (3.0 mL), to which ethyl 5-chlorosulfonyl-1-(2-trimethylsilylethoxymethyl)pyrazole-3-carboxylate; ethyl 5-chlorosulfonyl-2-(2-trimethylsilylethoxymethyl)pyrazole-3-carboxylate (599.6 mg, 1.625 mmol) and NaOtBu (248.5 mg, 2.586 mmol) were added. The resulting mixture was stirred at 100° C. for 2 h. It was cooled to room temperature, then 1 N HCl solution (3.0 mL) was added, followed by EtOAc (3.0 mL). The phases were vigorously mixed and then allowed to settle into two layers. The organic layer was filtered and purified by reverse phase preparative chromatography using a C₁₈ column and a gradient eluent of 1 to 99% acetonitrile in water containing 5 mM hydrochloric acid to give ˜45 mg of pure product and ˜8 mg of impure product (ca. 80% purity). Pure product analysis: ESI-MS m/z calc. 565.1582, found 566.2 (M+1)⁺; Retention time: 2.28 minutes (LC method A); ¹H NMR (500 MHz, dimethylsulfoxide-d₆) δ 12.67 (s, 1H), 7.35 (s, 1H), 7.26 (t, J 7.6 Hz, 1H), 7.21 (s, 1H), 7.13 (d, J=7.6 Hz, 2H), 5.77 (s, 2H), 4.30 (q, J=7.1 Hz, 2H), 3.47 (t, J=7.7 Hz, 2H), 1.92 (s, 6H), 1.27 (t, J=7.1 Hz, 3H), 0.73 (t, J=7.7 Hz, 2H),−0.17 (s, 9H).

Try 3: In a 100-mL round-bottomed flask, 4-chloro-6-(2,6-dimethylphenyl)pyrimidin-2-amine (1.0033 g, 4.293 mmol) was dissolved in DMF (10 mL), to which ethyl 5-chlorosulfonyl-1-(2-trimethylsilylethoxymethyl)pyrazole-3-carboxylate; ethyl 5-chlorosulfonyl-2-(2-trimethylsilylethoxymethyl)pyrazole-3-carboxylate (2.081 g, 5.641 mmol) and NaOtBu (0.9206 g, 9.579 mmol) were added. The resulting mixture was stirred at 100° C. for 5 h. It was cooled to room temperature, then 1 N HCl solution (20 mL) was added. The mixture was extracted with ethyl acetate (3×30 mL). The combined organic extracts were washed with water (80 mL) and saturated aqueous sodium chloride solution (80 mL), then dried over sodium sulfate, filtered, and evaporated in vacuo. The resulting brown gum was purified by silica gel chromatography (120 g of silica) using a gradient eluent of 1 to 60% ethyl acetate in hexanes to give somewhat pure (˜75% pure) product, ethyl 5-[[4-chloro-6-(2,6-dimethylphenyl)pyrimidin-2-yl]sulfamoyl]-1-(2-trimethylsilylethoxymethyl)pyrazole-3-carboxylate; ethyl 5-[[4-chloro-6-(2,6-dimethylphenyl)pyrimidin-2-yl]sulfamoyl]-2-(2-trimethylsilylethoxymethyl)pyrazole-3-carboxylate (198.9 mg, 8%). ESI-MS m/z calc. 565.1582, found 566.2 (M+1)⁺; Retention time: 2.28 minutes; LC method A.

### Step 5: 5-[[4-[(2R)-2-Amino-4-methyl-pentoxy]-6-(2,6-dimethylphenyl)pyrimidin-2-yl]sulfamoyl]-1-(2-trimethylsilylethoxymethyl)pyrazole-3-carboxylic acid and 5-[[4-[(2R)-2-amino-4-methyl-pentoxy]-6-(2,6-dimethylphenyl)pyrimidin-2-yl]sulfamoyl]-2-(2-trimethylsilylethoxymethyl)pyrazole-3-carboxylic Acid

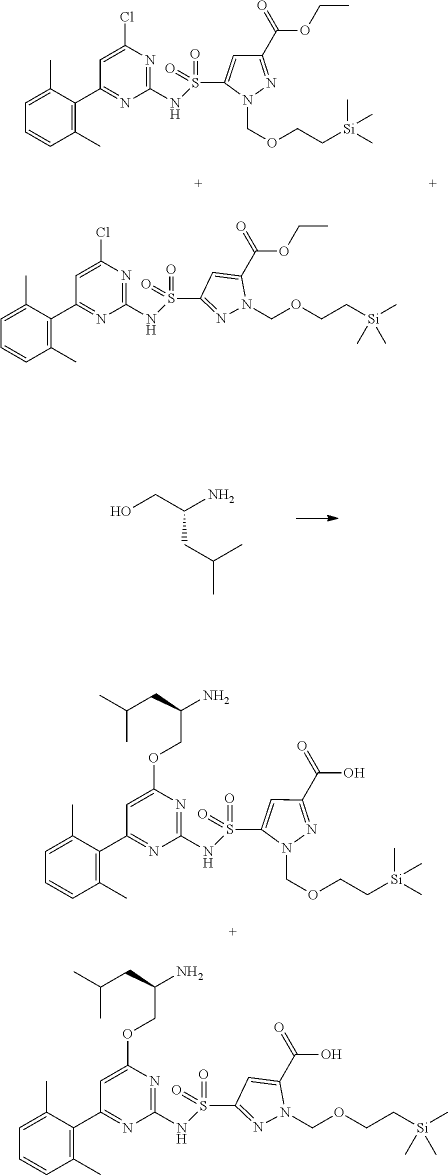

In a 3-mL vial, ethyl 5-[[4-chloro-6-(2,6-dimethylphenyl)pyrimidin-2-yl]sulfamoyl]-1-(2-trimethylsilylethoxymethyl)pyrazole-3-carboxylate; ethyl 5-[[4-chloro-6-(2,6-dimethylphenyl)pyrimidin-2-yl]sulfamoyl]-2-(2-trimethylsilylethoxymethyl)pyrazole-3-carboxylate (43.9 mg, 0.07754 mmol) was dissolved in THE (1.0 mL), to which (2R)-2-amino-4-methyl-pentan-1-ol (35.9 mg, 0.3063 mmol) and NaOtBu (45 mg, 0.4682 mmol) were added. The resulting mixture was stirred at room temperature for 1 h. After this time, 1 N HCl solution (1 mL) was added, followed by EtOAc (1 mL). The phases were vigorously mixed and then allowed to settle into two layers. The organic layer was filtered and purified by reverse phase preparative chromatography using a C₁₈ column and a gradient eluent of 1 to 99% acetonitrile in water containing 5 mM hydrochloric acid. The collected fractions were extracted with EtOAc/water to give ester intermediate (impure; 1.0 mg), as well as the desired product as a white solid: 5-[[4-[(2R)-2-amino-4-methyl-pentoxy]-6-(2,6-dimethylphenyl)pyrimidin-2-yl]sulfamoyl]-1-(2-trimethylsilylethoxymethyl)pyrazole-3-carboxylic acid; 5-[[4-[(2R)-2-amino-4-methyl-pentoxy]-6-(2,6-dimethylphenyl)pyrimidin-2-yl]sulfamoyl]-2-(2-trimethylsilylethoxymethyl)pyrazole-3-carboxylic acid (9.9 mg, 21%) ESI-MS m/z calc. 618.26556, found 619.3 (M+1)⁺; Retention time: 1.44 minutes (LC method A).

### Step 6: (10R)-15-(2,6-dimethylphenyl)-10-isobutyl-3,3-dioxo-12-oxa-3λ⁶-thia-2,5,6,9,16,17-hexazatricyclo[11.3.1.14,7]octadeca-1(17),4(18),6,13,15-pentaen-8-one (Compound 78)

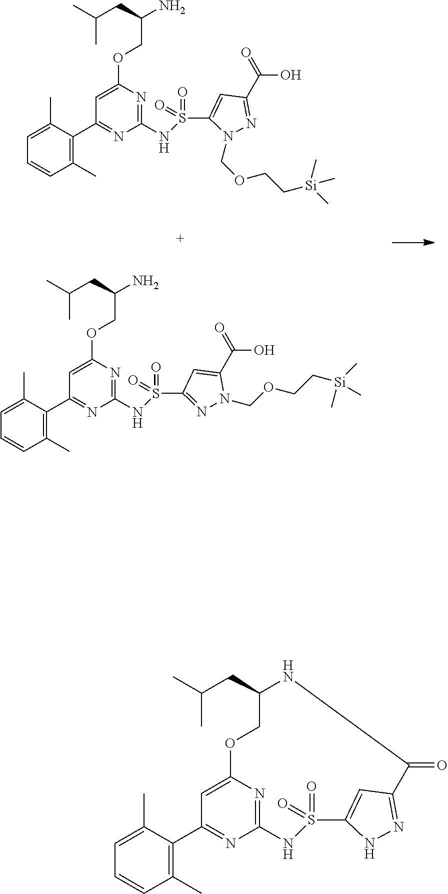 [SMILES: Cc1cccc(C)c1-c1cc(OC[C@H](N)CC(C)C)nc(NS(=O)(=O)c2cc(C(=O)O)nn2COCC[Si](C)(C)C)n1 ; Cc1cccc(C)c1-c1cc(OC[C@H](N)CC(C)C)nc(NS(=O)(=O)c2cc(C(=O)O)n(COCC[Si](C)(C)C)n2)n1 ; Cc1cccc(C)c1-c1cc2nc(n1)NS(=O)(=O)c1cc(n[nH]1)C(=O)N[C@H](CC(C)C)CO2]

In a 50-mL round-bottomed flask, 5-[[4-[(2R)-2-amino-4-methyl-pentoxy]-6-(2,6-dimethylphenyl)pyrimidin-2-yl]sulfamoyl]-1-(2-trimethylsilylethoxymethyl)pyrazole-3-carboxylic acid; 5-[[4-[(2R)-2-amino-4-methyl-pentoxy]-6-(2,6-dimethylphenyl)pyrimidin-2-yl]sulfamoyl]-2-(2-trimethylsilylethoxymethyl)pyrazole-3-carboxylic acid (20.3 mg, 0.03280 mmol) was dissolved in DMF (20 mL), to which DIPEA (100 μL, 0.5741 mmol) and HATU (25 mg, 0.06575 mmol) were added. The resulting solution was stirred at room temperature for 30 min, after which it was quenched with 1 N HCl solution (20 mL). The mixture was extracted with ethyl acetate (3×20 mL). The combined organic extracts was washed with water (50 mL) and saturated aqueous sodium chloride solution (50 mL), then dried over sodium sulfate, filtered, and evaporated in vacuo to give a yellow oil (˜30 mg, might still contain DMF). Both monomeric and dimeric products can be observed (in ca. 1:1 ratio). In a 3-mL vial, the crude product was dissolved in DCM (300 μL) and treated with TFA (300 μL, 3.894 mmol). The resulting solution was stirred at room temperature for 1.5 h, after which it was diluted with MeOH (500 μL), filtered, and purified by reverse phase preparative chromatography using a Cis column and a gradient eluent of 1 to 70% acetonitrile in water containing 5 mM hydrochloric acid to give (10R)-15-(2,6-dimethylphenyl)-10-isobutyl-3,3-dioxo-12-oxa-3λ⁶-thia-2,5,6,9,16,17-hexazatricyclo[11.3.1.14,7]octadeca-1(17),4(18),6,13,15-pentaen-8-one (4.4 mg, 29%)¹H NMR (500 MHz, dimethylsulfoxide-d₆) δ 8.16-7.92 (bs, 1H), 7.37-7.21 (bs, 1H), 7.28 (t, J 7.7 Hz, 1H), 7.15 (d, J 7.4 Hz, 2H), 6.29 (s, 1H), 4.86 (dd, J 10.7, 4.5 Hz, 1H), 3.58 (t, J 11.0 Hz, 1H), 2.69-2.58 (m, 1H), 2.17-1.96 (bs, 6H), 1.68-1.57 (m, 1H), 1.52-1.41 (m, 1H), 1.23-1.12 (m, 1H), 0.82 (d, J 6.7 Hz, 3H), 0.55 (d, J 5.8 Hz, 3H). Note: The broad peaks around 13-14 δ might be due to the sulfonamide NH but this cannot be confirmed from this spectrum. ESI-MS m/z calc. 470.1736, found 471.2 (M+1)⁺; Retention time: 1.3 minutes, LC method A.

### Example 77: Preparation of Compound 79 and Compound 80

### Step 1: tert-Butyl N-[1-(3,3-dimethylbutyl)-5-hydroxy-3-piperidyl]carbamate

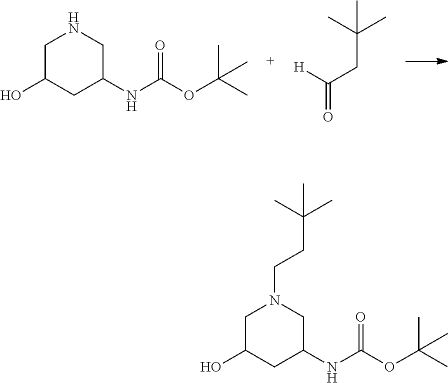

In a 20-mL vial, tert-butyl V-(5-hydroxy-3-piperidyl)carbamate (0.8538 g, 3.948 mmol), MeOH (9.0 mL) and 3,3-dimethylbutanal (1.50 mL, 11.95 mmol) were mixed together and kept at room temperature for 20 min. Then, sodium borohydride (0.1701 g, 4.496 mmol) was added in 5 portions (CAUTION: hydrogen gas evolution and exotherm). This reaction mixture was kept at room temperature for 10 min, upon which it was evaporated in vacuo to give a white foam. This crude product was purified by silica gel chromatography (40 g of silica, 0 to 100% gradient of ethyl acetate/hexanes followed by elution with 10% methanol/ethyl acetate) to give a light beige foam, tert-butyl N-[1-(3,3-dimethylbutyl)-5-hydroxy-3-piperidyl]carbamate (0.9447 g, 80%) ESI-MS m/z calc. 300.2413, found 301.3 (M+1)⁺; Retention time: 1.05 minutes, LC method A.

### Step 2: 5-Amino-1-(3,3-dimethylbutyl)piperidin-3-ol

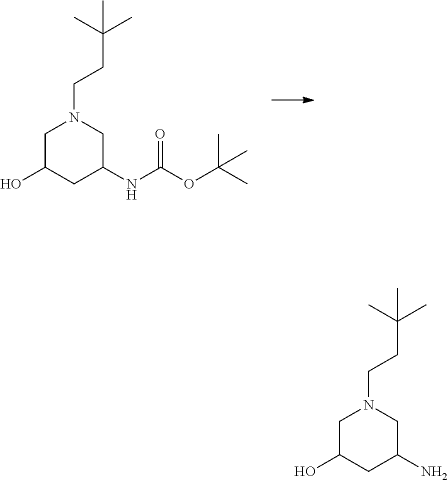 [SMILES: CC(C)(C)CCN1CC(O)CC(NC(=O)OC(C)(C)C)C1 ; CC(C)(C)CCN1CC(N)CC(O)C1]

In a 20-mL vial, tert-butyl N-[1-(3,3-dimethylbutyl)-5-hydroxy-3-piperidyl]carbamate (535.9 mg, 1.784 mmol) was dissolved in DCM (4.0 mL), to which TFA (4.0 mL, 51.92 mmol) was added. This solution was kept at 50° C. for 30 min, after which it was cooled to room to give 5-amino-1-(3,3-dimethylbutyl)piperidin-3-ol (hydrochloride salt) (206.1 mg, 49%) ESI-MS m z calc. 200.18886, found 201.2 (M+1)⁺; Retention time: 0.54 minutes, LC method A.

### Step 3: 5-(3,3-Dimethylbutyl)-19-(2,6-dimethylphenyl)-2-oxa-15λ⁶-thia-5,8,16,18,21-pentaazatetracyclo[15.3.1.13,7.110,14]tricosa-1(20),10,12,14(22),17(21),18-hexaene-9,15,15-trione, diastereomer 1 (Compound 80), and 5-(3,3-dimethylbutyl)-19-(2,6-dimethylphenyl)-2-oxa-15λ⁶-thia-5,8,16,18,21-pentaazatetracyclo[15.3.1.13,7.110,14]tricosa-1(20),10,12,14(22),17(21),18-hexaene-9,15,15-trione, diastereomer 2 (Compound 79)

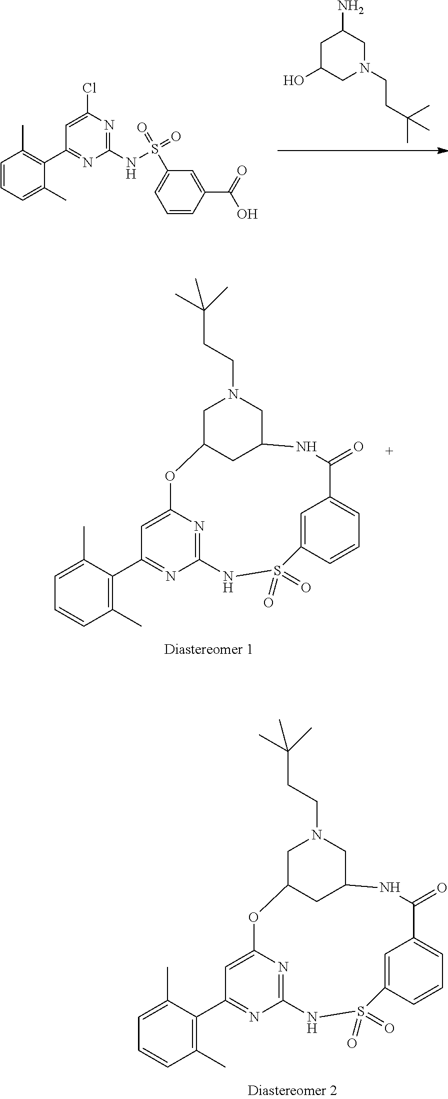 [SMILES: Cc1cccc(C)c1-c1cc(Cl)nc(NS(=O)(=O)c2cccc(C(=O)O)c2)n1 ; CC(C)(C)CCN1CC(N)CC(O)C1 ; Cc1cccc(C)c1-c1cc2nc(n1)NS(=O)(=O)c1cccc(c1)C(=O)NC1CC(CN(CCC(C)(C)C)C1)O2 ; Cc1cccc(C)c1-c1cc2nc(n1)NS(=O)(=O)c1cccc(c1)C(=O)NC1CC(CN(CCC(C)(C)C)C1)O2]

3-[[4-Chloro-6-(2,6-dimethylphenyl)pyrimidin-2-yl]sulfamoyl]benzoic acid (60 mg, 0.1436 mmol), 5-amino-1-(3,3-dimethylbutyl)piperidin-3-ol (hydrochloride salt) (36 mg, 0.1520 mmol), and sodium bicarbonate (60 mg, 0.7142 mmol) were combined in anhydrous dichloromethane (700 μL). DIEA (30 μL, 0.1916 mmol) was added and the reaction was stirred at room temperature two hours. The reaction mixture was concentrated to about 0.2 mL, diluted with DMSO and methanol, filtered and purified by reverse phase HPLC (1-50% ACN, HCl modifier). Two separate peaks were collected (presumably two different relative diastereomers); the first to elute gave 22 mg of a white solid 3-[[4-chloro-6-(2,6-dimethylphenyl)pyrimidin-2-yl]sulfamoyl]-N-[1-(3,3-dimethylbutyl)-5-hydroxy-3-piperidyl]benzamide (hydrochloride salt) (22 mg, 24%) ESI-MS m/z calc. 599.2333, found 600.3 (M+1)⁺; Retention time: 1.51 minutes (LC method A), and the second gave 35 mg of a white solid 3-[[4-chloro-6-(2,6-dimethylphenyl)pyrimidin-2-yl]sulfamoyl]-N-[1-(3,3-dimethylbutyl)-5-hydroxy-3-piperidyl]benzamide (hydrochloride salt) (35 mg, 38%) ESI-MS m/z calc. 599.2333, found 600.3 (M+1)⁺; Retention time: 1.53 minutes (LC method A).

The first to elute amide product from step 1 was combined with NaH (25 mg, 0.6251 mmol) in anhydrous NMP (6 mL) in a nitrogen-purged vial and heated to 70° C. for 2 hours. The reaction mixture was cooled to room temperature, quenched with a saturated ammonium chloride solution, diluted with water, and extracted 3× with ethyl acetate. The combined organics were washed with brine, dried over sodium sulfate and concentrated. The resulting crude material was purified by reverse phase HPLC (1-50% ACN, HCl modifier) to give 5-(3,3-dimethylbutyl)-19-(2,6-dimethylphenyl)-2-oxa-15λ⁶-thia-5,8,16,18,21-pentaazatetracyclo[15.3.1.13,7.110,14]tricosa-1(20),10,12,14(22),17(21),18-hexaene-9,15,15-trione (hydrochloride salt) (2 mg, 2%) ESI-MS m/z calc. 563.25665, found 564.3 (M+1)⁺; Retention time: 1.17 minutes

The second to elute amide product from step 1 was combined with NaH (25 mg, 0.6251 mmol) in anhydrous NMP (6 mL) in a nitrogen-purged vial and heated to 70° C. for 2 hours. The reaction mixture was cooled to room temperature, quenched with a saturated ammonium chloride solution, diluted with water, and extracted 3× with ethyl acetate. The combined organics were washed with brine, dried over sodium sulfate and concentrated. The resulting crude material was purified by reverse phase HPLC (1-50% ACN, HCl modifier) to give 5-(3,3-dimethylbutyl)-19-(2,6-dimethylphenyl)-2-oxa-15λ⁶-thia-5,8,16,18,21-pentaazatetracyclo[15.3.1.13,7.110,14]tricosa-1(20),10,12,14(22),17(21),18-hexaene-9,15,15-trione (hydrochloride salt) (3 mg, 3%) ESI-MS m/z calc. 563.25665, found 564.3 (M+1)⁺; Retention time: 1.2 minutes (LC method A).

### Example 78: Preparation of Compound 81 and Compound 82

### Step 1: 01-tert-Butyl 04-methyl 4-cyanopiperidine-1,4-dicarboxylate

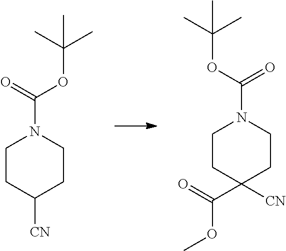 [SMILES: CC(C)(C)OC(=O)N1CCC(C#N)CC1 ; COC(=O)C1(C#N)CCN(C(=O)OC(C)(C)C)CC1]

To a solution of tert-butyl 4-cyanopiperidine-1-carboxylate (60.4 g, 0.287 mol) in anhydrous tetrahydrofuran (290 mL) was added 1 M solution of lithium bis(trimethylsilyl)amide in tetrahydrofuran (316 mL, 0.316 mol) dropwise to keep internal temperature below −70° C. After the addition was completed, the reaction solution was stirred for additional 30 minutes at this temperature. Methyl chloroformate (29.9 g, 0.316 mol) was added dropwise to keep internal temperature below −60° C. and the resulting solution was stirred at this temperature for 2 hours before it warmed up to ambient temperature. Saturated aqueous ammonium chloride solution (500 mL) was added and the organic layer was separated. The aqueous layer was extracted with diethyl ether (5×300 mL) and the combined organic layer was washed with 1M aqueous hydrogen chloride solution (300 mL), brine, dried over magnesium sulfate and concentrated to afford crude 1-tert-butyl 4-methyl 4-cyanopiperidine-1,4-dicarboxylate (81.8 g, 106%) as a pale-yellow oil, which was used directly in next step without purification. ¹H NMR (250 MHz, CDCl₃) δ (ppm): 4.14 (m, 2H), 3.85 (s, 3H), 3.11 (m, 2H), 2.00 (m, 4H), 1.46 (m, 9H).

### Step 2: tert-Butyl 4-cyano-4-(hydroxymethyl)piperidine-1-carboxylate

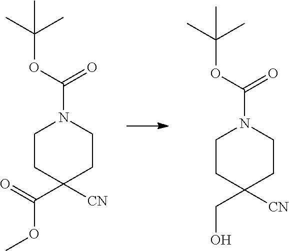 [SMILES: COC(=O)C1(C#N)CCN(C(=O)OC(C)(C)C)CC1 ; CC(C)(C)OC(=O)N1CCC(C#N)(CO)CC1]

Crude 1-tert-butyl 4-methyl 4-cyanopiperidine-1,4-dicarboxylate (81.8 g, 0.287 mol) were dissolved in methanol (430 mL) and the solution was cooled to 0° C. Sodium borohydride (13.0 g, 0.344 mol) was added in portions to maintain internal temperature below 5 C. The reaction solution was stirred at 0 C for 2.5 hours. A mixture of saturated aqueous ammonium chloride solution (150 mL) and 2M aqueous hydrogen chloride solution (150 mL) was added until pH value reached 7. Most of methanol was removed under reduced pressure and the residue was extracted with diethyl ether (5×250 mL). The combined organic layer was dried over magnesium sulfate and concentrated. The residue was purified by silica gel column chromatography using 0-50% hexanes-ethyl acetate. Fractions were combined and concentrated to about 200 mL volume. A formed precipitate was separated by filtration and discarded. The filtrate was further concentrated to afford crude material which was recrystallized from methyl isopropyl ketone to afford tert-butyl 4-cyano-4-(hydroxymethyl)piperidine-1-carboxylate (20.9 g, 30%) as a white solid. ¹H NMR (250 MHz, CDCl₃) δ (ppm): 4.12 (m, 2H), 3.64 (d, J 6.0 Hz, 2H), 3.01 (m, 2H), 2.85 (d, J 6.0 Hz, 1H), 1.94 (m, 2H), 1.50 (s, 9H), 1.41 (m, 2H). ESI-MS m/z calc. 240.15, found 241.5 (M1). Retention time: 3.24 minutes.

### Step 3: tert-Butyl 4-(aminomethyl)-4-(hydroxymethyl)piperidine-1-carboxylate

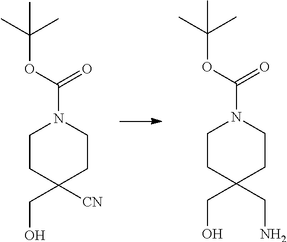 [SMILES: CC(C)(C)OC(=O)N1CCC(C#N)(CO)CC1 ; CC(C)(C)OC(=O)N1CCC(CN)(CO)CC1]

Freshly prepared Raney-Ni catalyst (2 g) was added to a solution of tert-butyl 4-cyano-4-(hydroxymethyl)piperidine-1-carboxylate (20.9 g, 87.1 mmol) in a 7 N ammonia solution in methanol (500 mL) and the reaction solution was hydrogenated under 65 PSI for 26 hours. The reaction mixture was filtered through Celite pad and the filtrate was concentrated. The residue was dried under high vacuum to afford tert-butyl 4-(aminomethyl)-4-(hydroxymethyl)piperidine-1-carboxylate (21.0 g, 100%) as a white solid. ¹H NMR (500 MHz, CDCl₃) δ (ppm): 3.69 (s, 2H), 3.54 (m, 2H), 3.22 (m, 2H), 2.82 (s, 2H), 1.53 (m, 2H), 1.45 (s, 9H), 1.31 (m, 2H). ESI-MS m/z calc. 244.18, found 245.2 (M1). Retention time: 1.25 minutes.

### Step 4: 4-({3-[4-Chloro-6-(2,6-dimethyl-phenyl)-pyrimidin-2-ylsulfamoyl]-benzoylamino}-methyl)-4-hydroxymethyl-piperidine-1-carboxylic acid tert-butyl ester

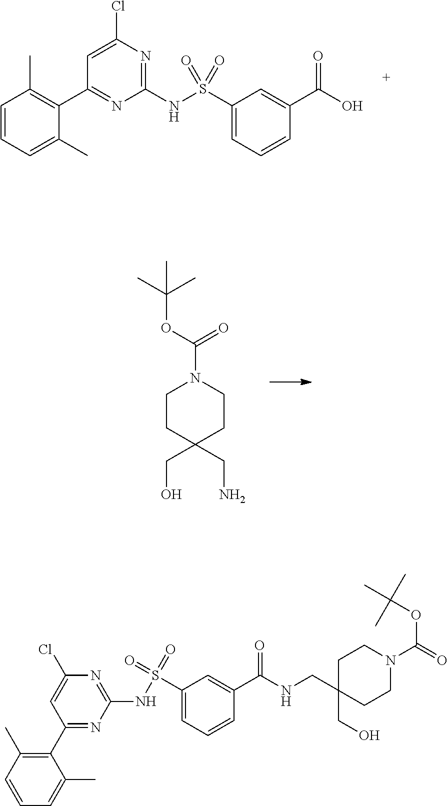 [SMILES: Cc1cccc(C)c1-c1cc(Cl)nc(NS(=O)(=O)c2cccc(C(=O)O)c2)n1 ; CC(C)(C)OC(=O)N1CCC(CN)(CO)CC1 ; Cc1cccc(C)c1-c1cc(Cl)nc(NS(=O)(=O)c2cccc(C(=O)NCC3(CO)CCN(C(=O)OC(C)(C)C)CC3)c2)n1]

To a solution of 3-[4-chloro-6-(2,6-dimethyl-phenyl)-pyrimidin-2-ylsulfamoyl]-benzoic acid (4.47 g, 10.7 mmol) and tert-butyl 4-(aminomethyl)-4-(hydroxymethyl)piperidine-1-carboxylate (2.61 g, 10.7 mmol) in anhydrous dichloromethane (55 mL) was added N,N′-diisopropylcarbodiimide (1.35 g, 10.7 mmol) and the resulting solution was stirred at ambient temperature for 21 hours. The reaction mixture was diluted with dichloromethane (400 mL) and 0.2M aqueous hydrogen chloride solution (60 mL). The organic layer was separated, washed with brine (3×50 mL), dried over sodium sulfate and concentrated. The residue was purified by silica gel column chromatography using 0-40% hexanes-acetone to furnish 4-({3-[4-Chloro-6-(2,6-dimethyl-phenyl)-pyrimidin-2-ylsulfamoyl]-benzoylamino}-methyl)-4-hydroxymethyl-piperidine-1-carboxylic acid tert-butyl ester (4.90 g, 71%) as a white solid. ESI-MS m/z calc. 643.22 found 644.1 (M1). Retention time: 5.70 minutes.

### Step 5: tert-Butyl 6-(2,6-dimethylphenyl)-2,2,14-trioxo-spiro[9-oxa-2λ⁶-thia-3,5,13,20-tetrazatricyclo[13.3.1.14,8]icosa-1(19),4(20),5,7,15,17-hexaene-11,4′-piperidine]-1′-carboxylate (Compound 81)

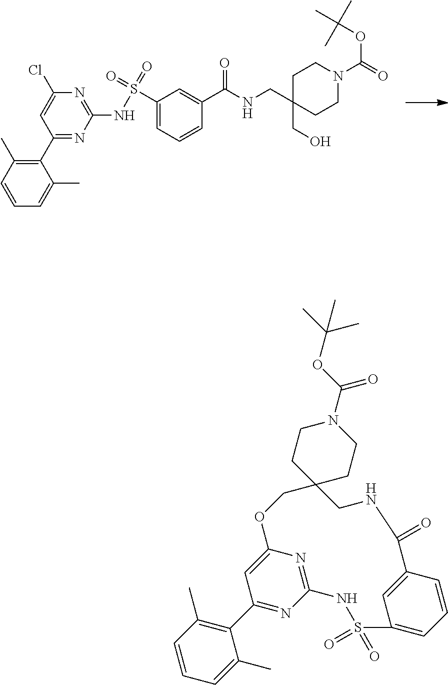 [SMILES: Cc1cccc(C)c1-c1cc(Cl)nc(NS(=O)(=O)c2cccc(C(=O)NCC3(CO)CCN(C(=O)OC(C)(C)C)CC3)c2)n1 ; Cc1cccc(C)c1-c1cc2nc(n1)NS(=O)(=O)c1cccc(c1)C(=O)NCC1(CCN(C(=O)OC(C)(C)C)CC1)CO2]

To a solution of 4-({3-[4-chloro-6-(2,6-dimethyl-phenyl)-pyrimidin-2-ylsulfamoyl]-benzoylamino}-methyl)-4-hydroxymethyl-piperidine-1-carboxylic acid tert-butyl ester (5.31 g, 8.26 mmol) in anhydrous tetrahydrofuran (1.65 L) was added slowly 60% sodium hydride dispersion in mineral oil (1.65 g, 41.28 mmol). The resulting solution was stirred at ambient temperature for 3 hours. Saturated aqueous sodium bicarbonate solution was added slowly until no further gas evaluation was observed. Most of organic solvents were removed under reduced pressure. 0.05 M aqueous hydrogen chloride solution was added until pH value was between 2 and 3 and the solution was extracted with ethyl acetate (3×300 mL). The combined organic layer was washed with brine (50 mL), dried over sodium sulfate and concentrated. The residue was purified by silica gel column chromatography using 0-50% hexanes-acetone to afforded tert-butyl 6′-(2,6-dimethylphenyl)-5′-oxospiro[piperidine-4,8′-10-oxa-3-thia-2,6-diaza-1(2,4)-pyrimidina-4(1,3)-benzenacyclodecaphane]-1-carboxylate 3′,3′-dioxide (3.32 g, 66%) as a white solid. ¹H NMR (500 MHz, CDCl₃) δ (ppm): 9.43 (s, 1H), 8.28 (s, 1H), 7.92 (m, 2H), 7.64 (m, 1H), 7.23 (t, J 7.5 Hz, 1H), 7.10 (d, J 7.5 Hz, 2H), 3.47 (m, 4H), 3.23 (m, 4H), 2.02 (s, 6H), 1.46 (m, 4H), 1.36 (s, 9H). ESI-MS m/z calc. 607.25, found 608.3 (M1). Retention time: 2.43 minutes.

### Step 6: 6-(2,6-Dimethylphenyl)-2,2-dioxo-spiro[9-oxa-2λ⁶-thia-3,5,13,20-tetrazatricyclo[13.3.1.14,8]icosa-1(19),4(20),5,7,15,17-hexaene-11,4′-piperidine]-14-one (Compound 82)

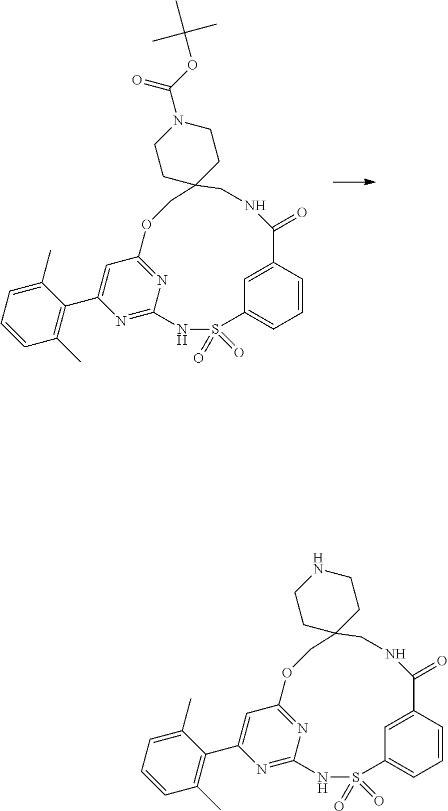 [SMILES: Cc1cccc(C)c1-c1cc2nc(n1)NS(=O)(=O)c1cccc(c1)C(=O)NCC1(CCN(C(=O)OC(C)(C)C)CC1)CO2 ; Cc1cccc(C)c1-c1cc2nc(n1)NS(=O)(=O)c1cccc(c1)C(=O)NCC1(CCNCC1)CO2]

tert-Butyl 6-(2,6-dimethylphenyl)-2,2,14-trioxo-spiro[9-oxa-2λ⁶-thia-3,5,13,20-tetrazatricyclo[13.3.1.14,8]icosa-1(19),4(20),5,7,15,17-hexaene-11,4′-piperidine]-1′-carboxylate (1.5 g, 99%) was analyzed before reacting. ¹H NMR (400 MHz, Chloroform-d) δ 9.40 (s, 1H), 8.17 (d, J 7.8 Hz, 1H), 7.96 (d, J 7.9 Hz, 1H), 7.60 (t, J 7.8 Hz, 1H), 7.46 (s, 1H), 7.22 (d, J 15.2 Hz, 1H), 7.08 (d, J 7.6 Hz, 2H), 6.35 (s, 1H), 4.38 (s, 2H), 3.64 (s, 4H), 3.39 (s, 2H), 2.03 (s, 6H), 1.66-1.57 (m, 4H), 1.46 (s, 9H), 1.29-1.25 (m, 1H), 0.90-0.84 (m, 1H). ESI-MS m/z calc. 607.24646, found 608.0 (M+1)⁺; Retention time: 1.68 minutes (LC method A).

TFA (5 mL, 64.90 mmol) was added to tert-butyl-(2,6-dimethylphenyl)-2,2,14-trioxo-spiro[9-oxa-2λ⁶-thia-3,5,13,20-tetrazatricyclo[13.3.1.14,8]icosa-1(19),4(20),5,7,15,17-hexaene-11,4′-piperidine]-1′-carboxylate (1.5 g, 2.468 mmol) in DCM (5 mL). The mixture was stirred at room temperature. Solvents were removed and the crude material was resuspended in DCM/toluene and the mixture was concentrated to dryness under reduced pressure (this step was repeated 3 times). A small amount was dissolved in DMSO and purified on reverse phase HPLC (Waters, HCl, 10-60% ACN-water) to give 6-(2,6-dimethylphenyl)-2,2-dioxo-spiro[9-oxa-2λ⁶-thia-3,5,13,20-tetrazatricyclo[13.3.1.14,8]icosa-1(19),4(20),5,7,15,17-hexaene-11,4′-piperidine]-14-one (1.7 g, 135%) ¹H NMR (400 MHz, DMSO-d₆) δ 9.43 (s, 1H), 8.82 (s, 2H), 8.46 (s, 1H), 7.95 (s, 2H), 7.65 (s, 1H), 7.25 (s, 1H), 7.12 (d, J 7.5 Hz, 2H), 6.53 (s, 1H), 4.64 (s, 1H), 3.46 (s, 2H), 3.12 (s, 4H), 2.04 (s, 6H), 1.78 (d, J 3.0 Hz, 2H), 1.58 (s, 2H). ESI-MS m/z calc. 507.19403, found 508.0 (M+1)⁺; Retention time: 0.88 minutes (LC method A).

### Example 79: Preparation of Compound 83

### Step 2: 6-(2,6-Dimethylphenyl)-2,2-dioxo-1′-[(3-phenoxy-2-thienyl)methyl]spiro[9-oxa-2λ⁶-thia-3,5,13,20-tetrazatricyclo[13.3.1.14,8]icosa-1(19),4(20),5,7,15,17-hexaene-11,4′-piperidine]-14-one (Compound 83)

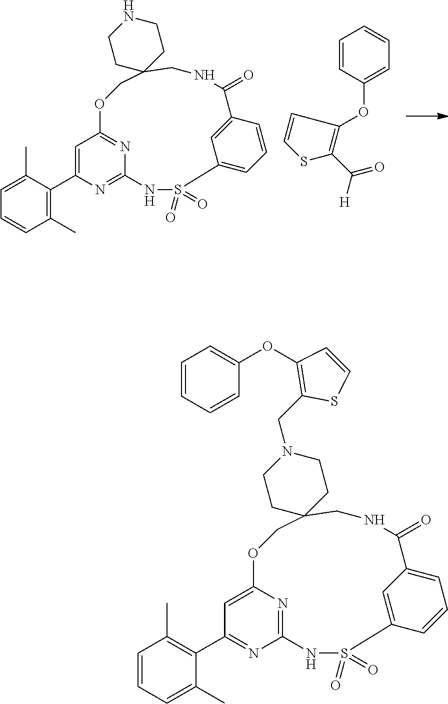

In a 3-mL vial, 6-(2,6-dimethylphenyl)-2,2-dioxo-spiro[9-oxa-2λ⁶-thia-3,5,13,20-tetrazatricyclo[13.3.1.14,8]icosa-1(19),4(20),5,7,15,17-hexaene-11,4′-piperidine]-14-one (trifluoroacetate salt) (20 mg, 0.03201 mmol) was dissolved in acetic acid (0.5 mL), to which 3-phenoxythiophene-2-carbaldehyde (approximately 19.61 mg, 0.09603 mmol) and sodium triacetoxyborohydride (approximately 20.35 mg, 0.09603 mmol) were added. This reaction mixture was stirred at room temperature for 1 h, then at 50° C. for 20 min. It was then cooled to room temperature, filtered and purified by reverse phase HPLC (1-70% acetonitrile in water using HCl as a modifier) to give 6-(2,6-dimethylphenyl)-2,2-dioxo-1′-[(3-phenoxy-2-thienyl)methyl]spiro[9-oxa-2λ⁶-thia-3,5,13,20-tetrazatricyclo[13.3.1.14,8]icosa-1(19),4(20),5,7,15,17-hexaene-11,4′-piperidine]-14-one (2.8 mg, 12%). ESI-MS m/z calc. 695.22363, found 696.0 (M+1)⁺; Retention time: 1.32 minutes; LC method A.

### Example 80: Preparation of Compound 84

### Step 1: 6-(2,6-Dimethylphenyl)-2,2-dioxo-1′-[6-(trifluoromethoxy)-1H-indole-2-carbonyl]spiro[9-oxa-2λ⁶-thia-3,5,13,20-tetrazatricyclo[13.3.1.14,8]icosa-1(19),4(20),5,7,15,17-hexaene-11,4′-piperidine]-14-one (Compound 84)

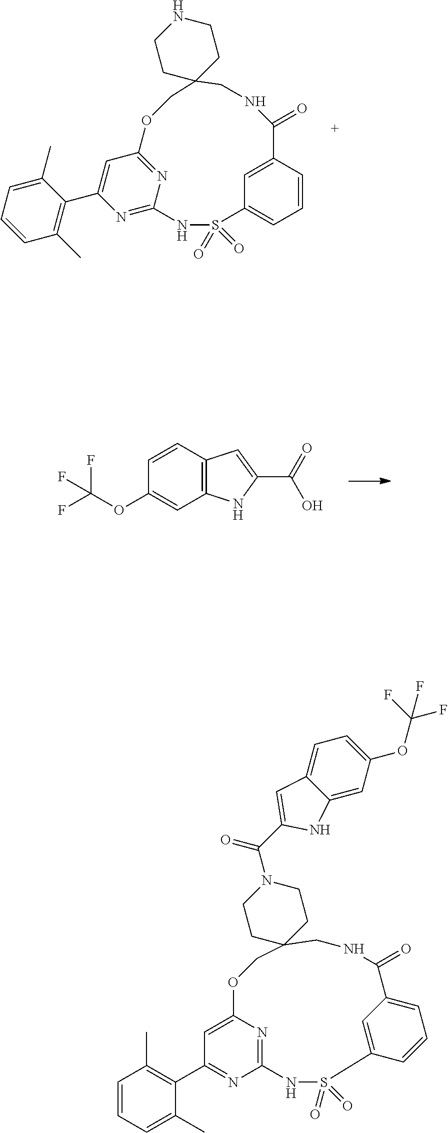 [SMILES: Cc1cccc(C)c1-c1cc2nc(n1)NS(=O)(=O)c1cccc(c1)C(=O)NCC1(CCNCC1)CO2 ; O=C(O)c1cc2ccc(OC(F)(F)F)cc2[nH]1 ; Cc1cccc(C)c1-c1cc2nc(n1)NS(=O)(=O)c1cccc(c1)C(=O)NCC1(CCN(C(=O)c3cc4ccc(OC(F)(F)F)cc4[nH]3)CC1)CO2]

A mixture of 6-(2,6-dimethylphenyl)-2,2-dioxo-spiro[9-oxa-2λ⁶-thia-3,5,13,20-tetrazatricyclo[13.3.1.14,8]icosa-1(19),4(20),5,7,15,17-hexaene-11,4′-piperidine]-14-one (20 mg, 0.03920 mmol), HATU (approximately 22.36 mg, 0.05880 mmol) in DMF (0.5 mL), diisopropyl ethyl amine (approximately 20.27 mg, 27.32 μL, 0.1568 mmol), and 6-(trifluoromethoxy)-1H-indole-2-carboxylic acid (approximately 14.42 mg, 0.05880 mmol) was agitated at 60° C. for 2 hours. The reaction mixture was filtered, and purified by reverse phase preparative chromatography using a C₁₈ column and a gradient eluent of acetonitrile in water containing 5 mM hydrochloric acid to give 6-(2,6-dimethylphenyl)-2,2-dioxo-1′-[6-(trifluoromethoxy)-1H-indole-2-carbonyl]spiro[9-oxa-2λ⁶-thia-3,5,13,20-tetrazatricyclo[13.3.1.14,8]icosa-1(19),4(20),5,7,15,17-hexaene-11,4′-piperidine]-14-one (12.9 mg, 44%) ESI-MS m/z calc. 734.21344, found 735.07 (M+1)⁺; Retention time. 1.84 minutes (LC method A).

### Example 81: Preparation of Compound 85

### Step 1: 1′-(Cyclohexylmethyl)-6-(2,6-dimethylphenyl)-2,2-dioxo-spiro[9-oxa-2λ⁶-thia-3,5,13,20-tetrazatricyclo[13.3.1.14,8]icosa-1(19),4(20),5,7,15,17-hexaene-11,4′-piperidine]-14-one (Compound 85)

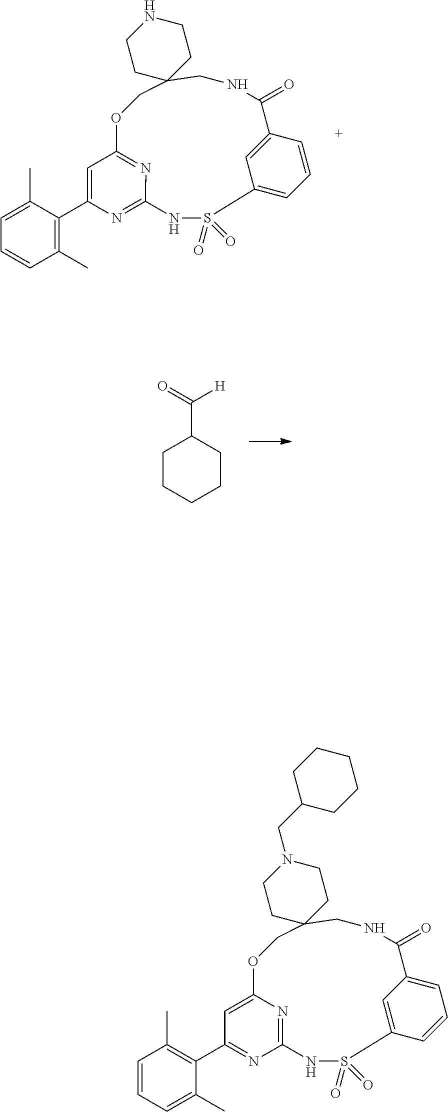 [SMILES: Cc1cccc(C)c1-c1cc2nc(n1)NS(=O)(=O)c1cccc(c1)C(=O)NCC1(CCNCC1)CO2 ; O=CC1CCCCC1 ; Cc1cccc(C)c1-c1cc2nc(n1)NS(=O)(=O)c1cccc(c1)C(=O)NCC1(CCN(CC3CCCCC3)CC1)CO2]

In a 3-mL vial, 6-(2,6-dimethylphenyl)-2,2-dioxo-spiro[9-oxa-2λ⁶-thia-3,5,13,20-tetrazatricyclo[13.3.1.14,8]icosa-1(19),4(20),5,7,15,17-hexaene-11,4′-piperidine]-14-one (trifluoroacetate salt) (20 mg, 0.03201 mmol) was dissolved in acetic acid (0.5 mL), to which cyclohexanecarbaldehyde (approximately 10.77 mg, 0.09603 mmol) and sodium triacetoxyborohydride (approximately 20.35 mg, 0.09603 mmol) were added. This reaction mixture was stirred at room temperature for 1 h, then at 50° C. for 20 min. It was then cooled to room temperature, filtered and purified by reverse phase HPLC (1-70% acetonitrile in water using HCl as a modifier) to give 1′-(cyclohexylmethyl)-6-(2,6-dimethylphenyl)-2,2-dioxo-spiro[9-oxa-2λ⁶-thia-3,5,13,20-tetrazatricyclo[13.3.1.14,8]icosa-1(19),4(20),5,7,15,17-hexaene-11,4′-piperidine]-14-one (3.2 mg, 16%). ESI-MS m/z calc. 603.2879, found 604.0 (M+1)⁺; Retention time: 1.15 minutes; (LC method A). ¹H NMR (400 MHz, DMSO-d₆) δ 9.58-9.22 (m, 2H), 8.67-8.26 (m, 1H), 7.95 (s, 2H), 7.66 (d, J=6.7 Hz, 1H), 7.26 (t, J=7.6 Hz, 1H), 7.13 (d, J=7.6 Hz, 2H), 6.75-6.37 (m, 1H), 4.62 (s, 2H), 3.58 (s, 1H), 3.42 (s, 2H), 3.29-3.13 (m, 3H), 3.13-3.01 (m, 1H), 2.99-2.88 (m, 2H), 2.14-1.93 (m, 7H), 1.88 (s, 1H), 1.82-1.72 (m, 3H), 1.72-1.50 (m, 4H), 1.34-1.07 (m, 3H), 1.02-0.87 (m, 2H).

### Example 82: Preparation of Compound 86

### Step 1: 1′-(3,3-Dimethylbutyl)-6-(2,6-dimethylphenyl)-2,2-dioxo-spiro[9-oxa-2λ⁶-thia-3,5,12,19-tetrazatricyclo[12.3.1.14,8]nonadeca-1(18),4(19),5,7,14,16-hexaene-11,4′-piperidine]-13-one (Compound 86)

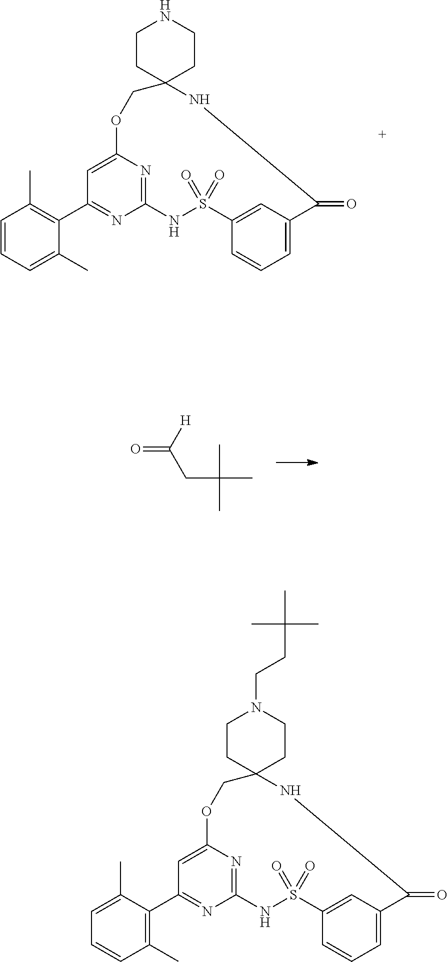

6-(2,6-Dimethylphenyl)-2,2-dioxo-spiro[9-oxa-2λ⁶-thia-3,5,12,19-tetrazatricyclo[12.3.1.14,8]nonadeca-1(18),4(19),5,7,14,16-hexaene-11,4′-piperidine]-13-one (hydrochloride salt) (25 mg, 0.04717 mmol) (with substantial impurities) was combined with the 3,3-dimethylbutanal (19 mg, 0.1897 mmol) in dichloromethane (0.3 mL). Sodium triacetoxyborohydride (60 mg, 0.2831 mmol) was added and the reaction was stirred for one hour at room temperature (for list 2 a second addition of carbonyl and sodium triacetoxyborohydride was necessary at this time, and the reaction was then stirred for an additional hour at room temperature.) The reaction was then partially concentrated, dissolved in 1.1 DMSO/methanol, filtered, and purified by reverse phase HPLC (1-50% ACN in water, HCl modifier, 15 min run, or 1-40% ACN for list 2) to give as white powder 1′-(3,3-dimethylbutyl)-6-(2,6-dimethylphenyl)-2,2-dioxo-spiro[9-oxa-2λ⁶-thia-3,5,12,19-tetrazatricyclo[12.3.1.14,8]nonadeca-1(18),4(19),5,7,14,16-hexaene-11,4′-piperidine]-13-one (hydrochloride salt) (5.3 mg, 18%). ESI-MS m/z calc. 577.2723, found 578.4 (M+1)⁺; Retention time: 1.09 minutes; LC method A.

### Example 83: Preparation of Compound 87 and Compound 88

### Step 1: 3-[[4-[(4-Amino-1-tert-butoxycarbonyl-3-piperidyl)oxy]-6-(2,6-dimethylphenyl)pyrimidin-2-yl]sulfamoyl]benzoic Acid

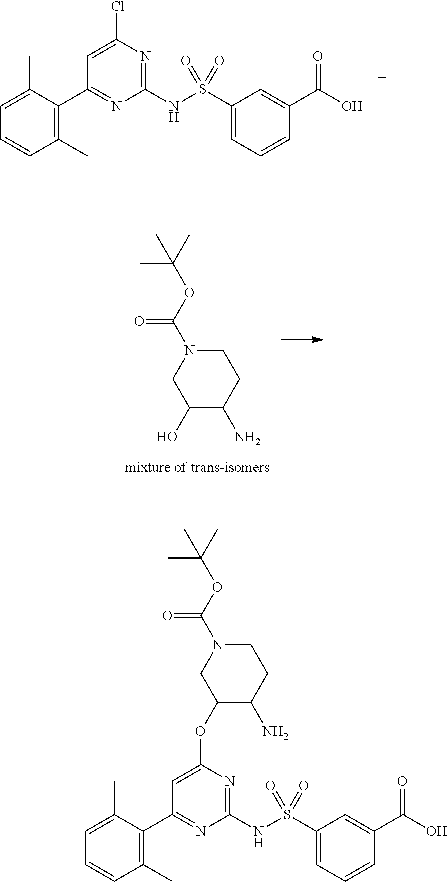 [SMILES: Cc1cccc(C)c1-c1cc(Cl)nc(NS(=O)(=O)c2cccc(C(=O)O)c2)n1 ; CC(C)(C)OC(=O)N1CCC(N)C(O)C1 ; Cc1cccc(C)c1-c1cc(OC2CN(C(=O)OC(C)(C)C)CCC2N)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (3.7 g, 8.855 mmol), tert-butyl (3R,4R)-4-amino-3-hydroxy-piperidine-1-carboxylate (2.1 g, 9.710 mmol) (mixture of trans isomers), and sodium t-butoxide (2.6 g, 27.05 mmol) in THE (45 mL) was stirred for 18 hours. The reaction was acidified with 1 M citric acid, diluted with water, and extracted with ethyl acetate. The combined extracts were washed with water, dried over sodium sulfate, and evaporated under vacuum to give give crude 3-[[4-[(4-amino-1-tert-butoxycarbonyl-3-piperidyl)oxy]-6-(2,6-dimethylphenyl)pyrimidin-2-yl]sulfamoyl]benzoic acid, as a mixture of trans-isomers (4.8 g, 91%) ESI-MS m/z calc. 597.2257, found 598.2 (M+1)⁺; Retention time: 0.48 minutes, LC method D.

### Step 2: tert-Butyl 20-(2,6-dimethylphenyl)-10,16,16-trioxo-2-oxa-16λ⁶-thia-5,9,17,19,22-pentaazatetracyclo[16.3.1.111,15.03,8]tricosa-1(21),11(23),12,14,18(22),19-hexaene-5-carboxylate, diastereomer 1 (Compound 88), and tert-butyl 20-(2,6-dimethylphenyl)-10,16,16-trioxo-2-oxa-16λ⁶-thia-5,9,17,19,22-pentaazatetracyclo[16.3.1.111,15.03,8]tricosa-1(21),11(23),12,14,18(22),19-hexaene-5-carboxylate, diastereomer 2 (Compound 87)

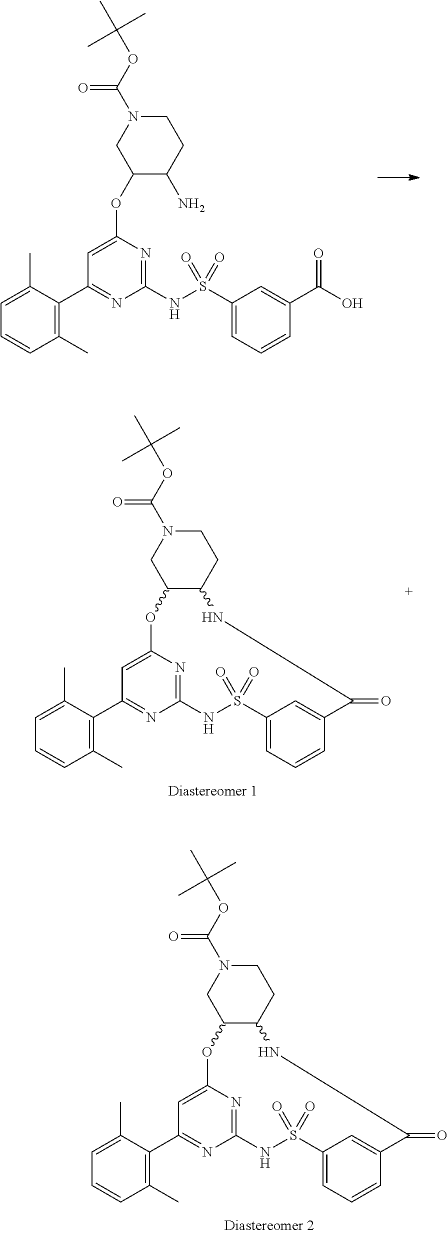 [SMILES: Cc1cccc(C)c1-c1cc(OC2CN(C(=O)OC(C)(C)C)CCC2N)nc(NS(=O)(=O)c2cccc(C(=O)O)c2)n1 ; Cc1cccc(C)c1-c1cc2nc(n1)NS(=O)(=O)c1cccc(c1)C(=O)NC1CCN(C(=O)OC(C)(C)C)CC1O2 ; Cc1cccc(C)c1-c1cc2nc(n1)NS(=O)(=O)c1cccc(c1)C(=O)NC1CCN(C(=O)OC(C)(C)C)CC1O2]

A solution of crude 3-[[4-[(4-amino-1-tert-butoxycarbonyl-3-piperidyl)oxy]-6-(2,6-dimethylphenyl)pyrimidin-2-yl]sulfamoyl]benzoic acid (4.8 g, 8.031 mmol) (mixture of trans-isomers), HATU (4.6 g, 12.10 mmol), and DIEA (4.2 mL, 24.11 mmol) in DMF (0.4 L) was stirred for 19 hours. The reaction was acidified with 1 M citric acid, diluted with water, and extracted with ethyl acetate. The combined extracts were washed with brine and water, dried over sodium sulfate, and evaporated under vacuum. The residue was purified by silica gel column chromatography with 0-5% methanol in dichloromethane to give 1.4 g of a clean mixture of trans-isomers. The mixture was purified by chiral SFC(Phenomenex LUX-1 column eluting with 28% MeOH (no modifier), 78% CO₂), to give diastereomer 1 tert-butyl 20-(2,6-dimethylphenyl)-10,16,16-trioxo-2-oxa-16λ⁶-thia-5,9,17,19,22-pentaazatetracyclo[16.3.1.111,15.03,8]tricosa-1(21),11(23),12,14,18(22),19-hexaene-5-carboxylate (0.56 g, 24%) ESI-MS m/z calc. 579.21515, found 580.2 (M+1)⁺; Retention time: 1.49 minutes (LC method A); ¹H NMR (400 MHz, DMSO-d₆) δ 12.96 (s, 1H), 8.51 (s, 1H), 8.35 (dd, J 28.4, 10.4 Hz, 1H), 7.91 (d, J 7.5 Hz, 1H), 7.76-7.59 (m, 2H), 7.26 (t, J 7.6 Hz, 1H), 7.12 (d, J 7.7 Hz, 2H), 6.16-5.89 (m, 1H), 5.42-5.25 (m, 1H), 4.27-4.12 (m, 1H), 3.83 (dd, J 30.6, 13.5 Hz, 1H), 3.70-3.49 (m, 2H), 3.49-3.35 (m, 1H), 2.06 (s, 6H), 1.88-1.73 (m, 1H), 1.38 (s, 9H), 1.37-1.32 (m, 1H) (first peak off the column); and diastereomer 2 tert-butyl 20-(2,6-dimethylphenyl)-10,16,16-trioxo-2-oxa-16λ⁶-thia-5,9,17,19,22-pentaazatetracyclo[16.3.1.111,15.03,8]tricosa-1(21),11(23),12,14,18(22),19-hexaene-5-carboxylate (0.57 g, 24%) ESI-MS m/z calc. 579.21515, found 580.2 (M+1)⁺; Retention time: 1.49 minutes (LC method A), ¹H NMR (400 MHz, DMSO-d₆) δ 12.94 (s, 1H), 8.51 (s, 1H), 8.35 (dd, J 28.4, 10.4 Hz, 1H), 7.91 (d, J 7.5 Hz, 1H), 7.78-7.60 (m, 2H), 7.26 (t, J 7.6 Hz, 1H), 7.12 (d, J 7.6 Hz, 2H), 6.13-5.88 (m, 1H), 5.41-5.26 (m, 1H), 4.26-4.12 (m, 1H), 3.83 (dd, J 29.8, 13.5 Hz, 1H), 3.69-3.50 (m, 2H), 3.50-3.35 (m, 1H), 2.06 (s, 6H), 1.89-1.74 (m, 1H), 1.38 (s, 9H), 1.37-1.33 (m, 1H) (second peak off the column). Both were obtained as pure trans-isomers and colorless solids.

### Example 84: Preparation of Compound 89 and Compound 90

### Step 1: 20-(2,6-Dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione

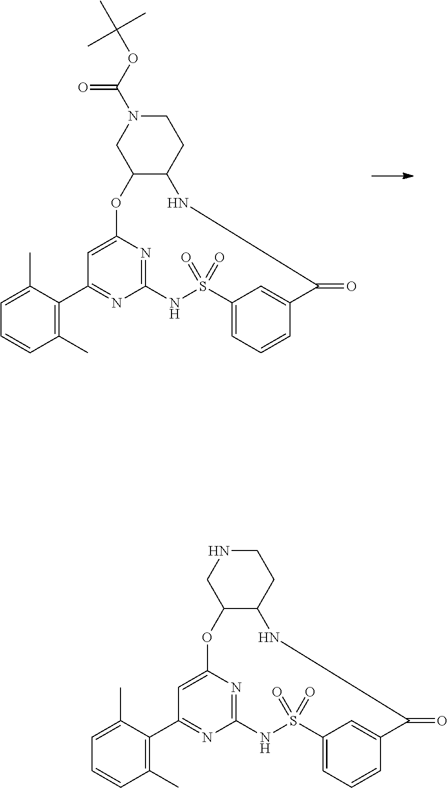 [SMILES: Cc1cccc(C)c1-c1cc2nc(n1)NS(=O)(=O)c1cccc(c1)C(=O)NC1CCN(C(=O)OC(C)(C)C)CC1O2 ; Cc1cccc(C)c1-c1cc2nc(n1)NS(=O)(=O)c1cccc(c1)C(=O)NC1CCNCC1O2]

A solution of tert-butyl 20-(2,6-dimethylphenyl)-10,16,16-trioxo-2-oxa-16λ⁶-thia-5,9,17,19,22-pentaazatetracyclo[16.3.1.111,15.03,8]tricosa-1(21),11(23),12,14,18(22),19-hexaene-5-carboxylate (mixture of trans isomers) (0.13 g, 0.2243 mmol) in HCl (2 mL of 4 M, 8.00 mmol) (in dioxane) was stirred for 16 hours. The solids were triturated with diethylether and dried under vacuum to give 20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione (hydrochloride salt) (0.11 g, 95%) as a colorless solid. ESI-MS m/z calc. 479.16272, found 480.2 (M+1)⁺; Retention time: 0.32 minutes (LC method D).

### Step 2: 5-(3,3-Dimethylbutyl)-20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione, diastereomer 1 (Compound 89), 5-(3,3-dimethylbutyl)-20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione, diastereomer 2 (Compound 90)

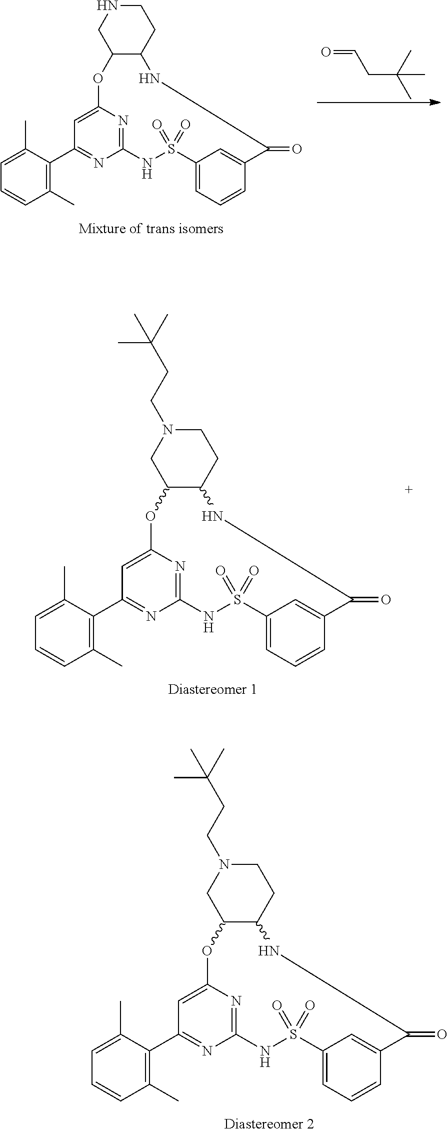

A solution of 20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione (hydrochloride salt) (55 mg, 0.1066 mmol) (mixture of trans-isomers), 3,3-dimethylbutanal (41 μL, 0.3267 mmol), and sodium triacetoxyborohydride (0.11 g, 0.5190 mmol) in dichloromethane (0.6 mL) was stirred for an hour. The reaction was quenched with 1 M HCl and purified by reverse-phase HPLC-MS (1%-99% acetonitrile/water (5 mM HCl)) to give 11 mg of a mixture of trans-isomers. The mixture was re-purified with the method below to give 5-(3,3-dimethylbutyl)-20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione, diastereomer 1 (2.5 mg, 8%) ESI-MS m/z calc. 563.25665, found 564.2 (M+1)⁺; Retention time: 1.03 minutes (LC method A); and 5-(3,3-dimethylbutyl)-20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione, diastereomer 2 (2.5 mg, 8%) ESI-MS m z calc. 563.25665, found 564.2 (M+1)⁺; Retention time: 1.05 minutes (LC method A), as pure trans diastereomers.

Samples were purified using a normal phase SFC-MS method using a (R,R)-Whelk-O column (150×2.1 mm, 3.5 m particle size) sold by Regis Technologies (pn: 780230), and a gradient run from 5-80% mobile phase B over 10 minutes. Mobile phase A=CO₂. Mobile phase B=MeOH (20 mM NH₃). Flow rate=0.7 mL/min [20 mM NH₃]. Injection volume=2.0 μL, and column temperature=55° C.

### Example 85: Preparation of Compound 91

### Step 1: 20-(2,6-Dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione, diastereomer 2

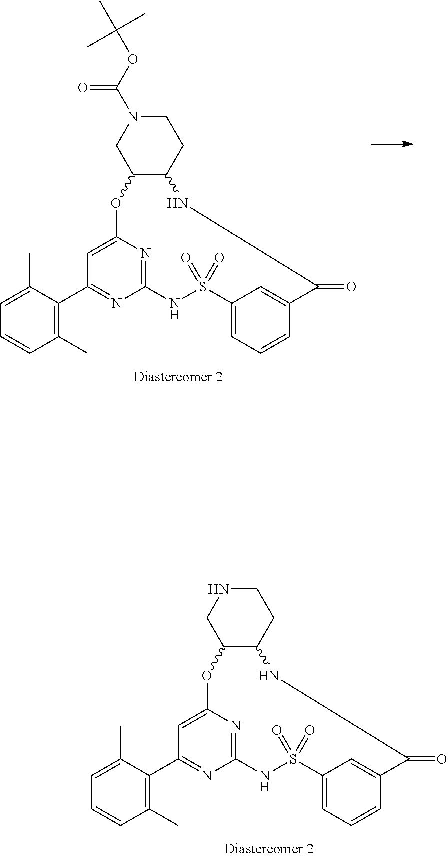 [SMILES: Cc1cccc(C)c1-c1cc2nc(n1)NS(=O)(=O)c1cccc(c1)C(=O)NC1CCN(C(=O)OC(C)(C)C)CC1O2 ; Cc1cccc(C)c1-c1cc2nc(n1)NS(=O)(=O)c1cccc(c1)C(=O)NC1CCNCC1O2]

A solution of tert-butyl 20-(2,6-dimethylphenyl)-10,16,16-trioxo-2-oxa-16λ⁶-thia-5,9,17,19,22-pentaazatetracyclo[16.3.1.111,15.03,8]tricosa-1(21),11(23),12,14,18(22),19-hexaene-5-carboxylate (trans isomer, diastereomer 2) (0.53 g, 0.905 mmol) in HCl (8 mL of 4 M, 332.00 mmol) (in dioxane) was stirred for 3 hours, and the solvents was removed under vacuum. The solids were triturated with diethylether and dried under vacuum to give 20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione (hydrochloride salt) (0.50 g, 107%) as a colorless solid (diastereomer 2). ESI-MS m/z calc. 479.16272, found 480.2 (M+1)⁺; Retention time: 0.31 minutes (LC method D).

### Step 2: 5-[(4-tert-butylphenyl)methyl]-20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione (Compound 91)

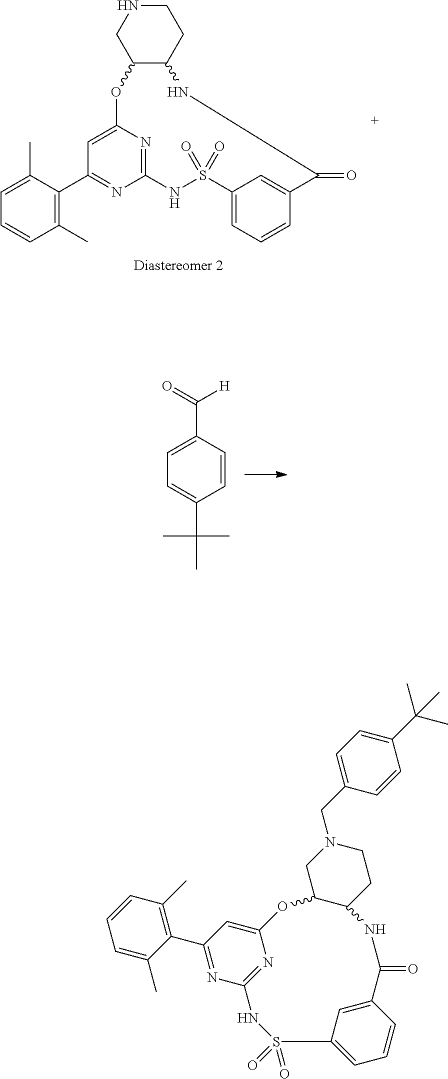 [SMILES: Cc1cccc(C)c1-c1cc2nc(n1)NS(=O)(=O)c1cccc(c1)C(=O)NC1CCNCC1O2 ; CC(C)(C)c1ccc(C=O)cc1 ; Cc1cccc(C)c1-c1cc2nc(n1)NS(=O)(=O)c1cccc(c1)C(=O)NC1CCN(Cc3ccc(C(C)(C)C)cc3)CC1O2]

A solution of 20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione (hydrochloride salt) diastereomer 2, (25.80 mg, 0.05 mmol), 4-tert-butylbenzaldehyde (approximately 24.33 mg, 25.11 μL, 0.1500 mmol), and sodium triacetoxyborohydride (approximately 42.39 mg, 0.2000 mmol) in dichloromethane (500.0 μL) was stirred for one hour. The reaction was stirred with methanol, the volatiles were removed under vacuum, and the residue was purified by reverse-phase HPLC-MS (20%-80% acetonitrile/water (5 mM HCl)) to give 5-[(4-tert-butylphenyl)methyl]-20-(2,6-dimethylphenyl)-2-oxa-16λ⁶-thia-5,9,17,19,22-pentaazatetracyclo[16.3.1.111,15.03,8]tricosa-1(21),11(23),12,14,18(22),19-hexaene-10,16,16-trione (hydrochloride salt) (22.8 mg, 68%) as a colorless solid. ESI-MS m/z calc. 625.2723, found 626.4 (M+1)⁺; Retention time: 1.29 minutes; LC method A.

### Example 86: Preparation of Compound 92

### Step 1: 3-[1-(Trifluoromethyl)cyclopropyl]propanal

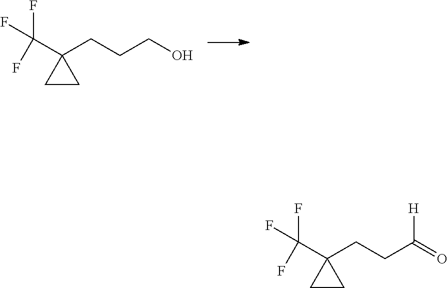 [SMILES: OCCCC1(C(F)(F)F)CC1 ; O=CCCC1(C(F)(F)F)CC1]

Dess-Martin periodinane (880 mg, 2.075 mmol) was added to a stirred solution of 3-[1-(trifluoromethyl)cyclopropyl]propan-1-ol (350 mg, 1.665 mmol) in anhydrous methylene chloride (10 mL) at 0° C. (ice-water bath) under nitrogen. After 15 min, the bath was removed, and the reaction was allowed to warm to ambient temperature and stirring continued for another 3 h. The reaction was diluted with ether (60 mL) and saturated aqueous sodium bicarbonate (20 mL) was added slowly (to mitigate CO₂ gas evolution). Then sodium thiosulfate (10 mL) was added and stirred at ambient temperature for 30 min. The layers were separated, and the aqueous layer was extracted with ether (2×20 mL). The combined organics were washed with brine (20 mL), dried over anhydrous sodium sulfate, filtered and concentrated under reduced pressure (pressure set at 300 mbar) to afford 3-[1-(trifluoromethyl)cyclopropyl]propanal (250 mg, 90%) as a yellow oil. ¹H NMR (400 MHz, Benzene-d₆) δ 9.15 (s, 1H), 1.99-1.90 (m, 2H), 1.52-1.44 (m, 2H), 0.68-0.59 (m, 2H), 0.00 (dd, J=2.5, 1.6 Hz, 2H).

### Step 2: 2-(Benzylamino)-4-[1-(trifluoromethyl)cyclopropyl]butanenitrile

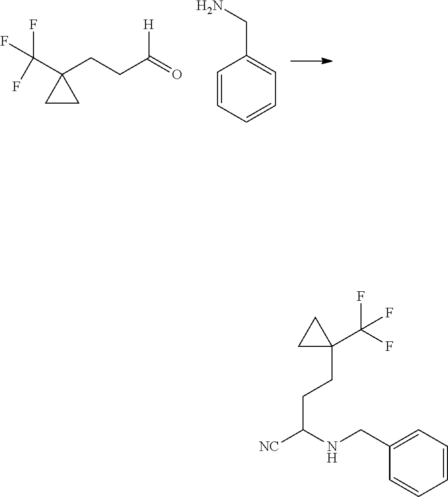

To a stirring solution of 3-[1-(trifluoromethyl)cyclopropyl]propanal (854 mg, 5.140 mmol) in acetonitrile (50.09 mL) under nitrogen atmosphere was added benzylamine (561.5 μL, 5.141 mmol) and trimethylsilylformonitrile (822.4 μL, 6.168 mmol). bromo(dimethyl)sulfonium bromide (114.1 mg, 0.5141 mmol) was then added and the mixture was stirred 2 h. Removed 90% of the acetonitrile by rotary evaporation then added water (50.09 mL). Extracted the resulting mixture with EtOAc (3×), combined org. phases, dried (sodium sulfate), filtered and conc. to light tan oil which became a light tan solid on the high vacuum pump, 2-(benzylamino)-4-[1-(trifluoromethyl)cyclopropyl]butanenitrile (1.33 g, 92%) ESI-MS m/z calc. 282.13437, found 283.0 (M+1)⁺; Retention time: 0.56 minutes, LC method D.

### Step 3: 2-(Benzylamino)-4-[1-(trifluoromethyl)cyclopropyl]butanoic acid

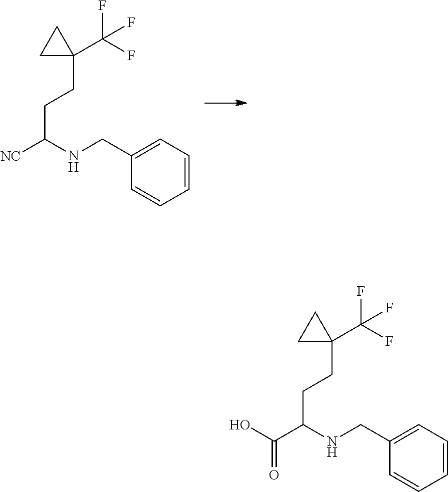 [SMILES: N#CC(CCC1(C(F)(F)F)CC1)NCc1ccccc1 ; O=C(O)C(CCC1(C(F)(F)F)CC1)NCc1ccccc1]

To a stirring solution of 2-(benzylamino)-4-[1-(trifluoromethyl)cyclopropyl]butanenitrile (1.33 g, 4.711 mmol) in acetic acid (897.3 μL, 15.78 mmol) in a vial was added HCl (8.96 mL of 37% w/v, 90.92 mmol) and the vial was capped. The mixture was stirred and heated in an aluminum block at 95° C. for 2 d. The mixture was transferred to a round bottom flask using MeOH and was concentrated by rotary evaporation, including treatment with diethyl ether and removing the solvents three times to give 2-(benzylamino)-4-[1-(trifluoromethyl)cyclopropyl]butanoic acid as a light tan solid that was dried thoroughly on the high vacuum pump then taken directly to the next step. (1.432 g, 100%) ESI-MS m/z calc. 301.12897, found 302.1 (M+1)⁺; Retention time: 0.37 minutes, LC method D.

### Step 4: 2-(Benzylamino)-4-[1-(trifluoromethyl)cyclopropyl]butan-1-ol

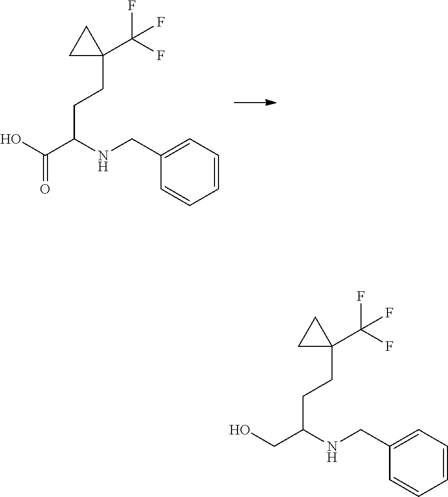 [SMILES: O=C(O)C(CCC1(C(F)(F)F)CC1)NCc1ccccc1 ; OCC(CCC1(C(F)(F)F)CC1)NCc1ccccc1]

To a stirring solution of 2-(benzylamino)-4-[1-(trifluoromethyl)cyclopropyl]butanoic acid (1.432 g, 4.705 mmol) in THE (28.36 mL) under nitrogen atmosphere at 0° C. was slowly added LAH (733.3 mg, 18.82 mmol) and the resulting mixture was stirred at 0° C. for 2 min then allowed to warm to rt and was stirred 75 min. Cooled to 0° C. and quenched by the addition of water (1.410 mL, 78.27 mmol), then KOH (1.411 mL of 15% w/v, 3.772 mmol) then water (2.819 mL, 156.5 mmol). Warmed to rt, added Celite and stirred 5 min then filtered over Celite eluting with ether. The ethereal filtrate was then dried (magnesium sulfate), filtered and concentrated the filtrate by rotary evaporation to give 2-(benzylamino)-4-[1-(trifluoromethyl)cyclopropyl]butan-1-ol (1.5146 g, 100%) as an orange oil which was used directly in the next step. ESI-MS m/z calc. 287.1497, found 288.0 (M+1)⁺; Retention time: 0.39 minutes, LC method D.

### Step 5: 3-[[4-[2-(Benzylamino)-4-[1-(trifluoromethyl)cyclopropyl]butoxy]-6-(2,6-dimethylphenyl)pyrimidin-2-yl]sulfamoyl]benzoic Acid

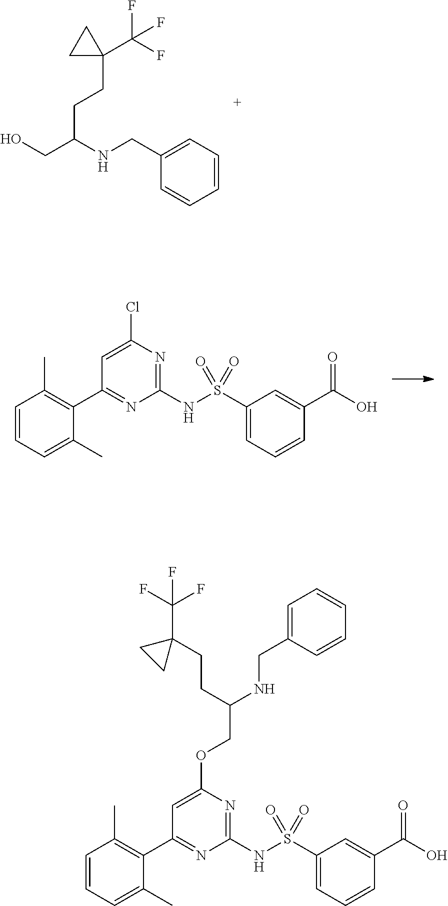 [SMILES: OCC(CCC1(C(F)(F)F)CC1)NCc1ccccc1 ; Cc1cccc(C)c1-c1cc(Cl)nc(NS(=O)(=O)c2cccc(C(=O)O)c2)n1 ; Cc1cccc(C)c1-c1cc(OCC(CCC2(C(F)(F)F)CC2)NCc2ccccc2)nc(NS(=O)(=O)c2cccc(C(=O)O)c2)n1]

To a stirring solution of 3-[[4-chloro-6-(2,6-dimethylphenyl)pyrimidin-2-yl]sulfamoyl]benzoic acid (647.3 mg, 1.549 mmol) and 2-(benzylamino)-4-[1-(trifluoromethyl)cyclopropyl]butan-1-ol (500 mg, 1.549 mmol) in THE (9.79 mL) at 0° C. was added KOtBu (770.8 μL, 6.196 mmol) and the mixture was stirred at 50° C. for 20 min then removed the THF by rotary evaporation, dissolved the residue in DMSO, filtered and chromatographed on a 275 g Reverse Phase Column eluting with 20-100% ACN/Water giving 3-[[4-[2-(benzylamino)-4-[1-(trifluoromethyl)cyclopropyl]butoxy]-6-(2,6-dimethylphenyl)pyrimidin-2-yl]sulfamoyl]benzoic acid (670 mg, 65%) ESI-MS m/z calc. 668.228, found 0.54 (M+1)⁺; Retention time: 669.1 minutes. ESI-MS m/z calc. 668.228, found 0.54 (M+1)⁺; Retention time: 669.1 minutes; LC method D.

### Step 6: 3-[[4-[2-amino-4-[1-(trifluoromethyl)cyclopropyl]butoxy]-6-(2,6-dimethylphenyl)pyrimidin-2-yl]sulfamoyl]benzoic Acid

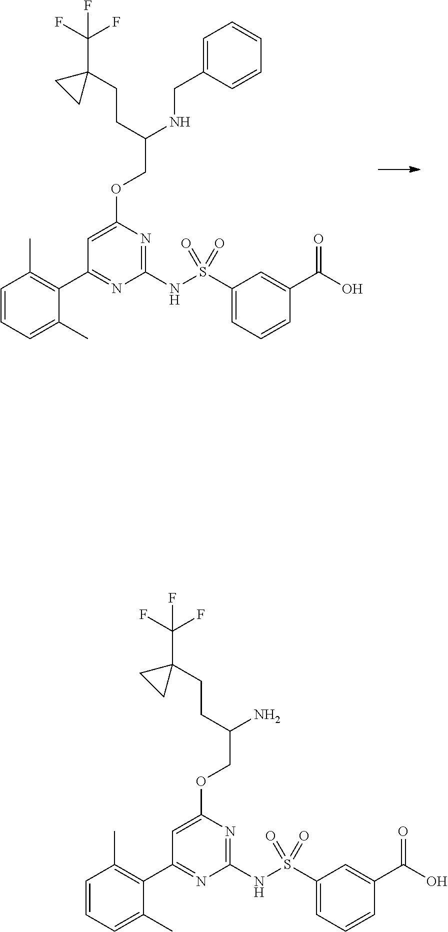 [SMILES: Cc1cccc(C)c1-c1cc(OCC(CCC2(C(F)(F)F)CC2)NCc2ccccc2)nc(NS(=O)(=O)c2cccc(C(=O)O)c2)n1 ; Cc1cccc(C)c1-c1cc(OCC(N)CCC2(C(F)(F)F)CC2)nc(NS(=O)(=O)c2cccc(C(=O)O)c2)n1]

A mixture of 3-[[4-[2-(benzylamino)-4-[1-(trifluoromethyl)cyclopropyl]butoxy]-6-(2,6-dimethylphenyl)pyrimidin-2-yl]sulfamoyl]benzoic acid (hydrochloride salt) (359 mg, 0.5368 mmol) and palladium(2+); dihydroxide (37.69 mg of 20% w/w, 0.05368 mmol) in Ethanol (8.0 mL) and HCl (1.1 mL of 1 M, 1.100 mmol) was purged with hydrogen gas (1 mg, 0.4961 mmol) and vigorously stirred under a hydrogen atmosphere for 6 hours. The reaction was filtered and concentrated under vacuum to give 3-[[4-[2-amino-4-[1-(trifluoromethyl)cyclopropyl]butoxy]-6-(2,6-dimethylphenyl)pyrimidin-2-yl]sulfamoyl]benzoic acid (hydrochloride salt) (303 mg, 92%) ESI-MS m/z calc. 578.1811, found 579.1 (M+1)⁺; Retention time: 1.14 minutes as a solid, LC method A.

### Step 7: 6-(2,6-dimethylphenyl)-2,2-dioxo-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one (Compound 92)

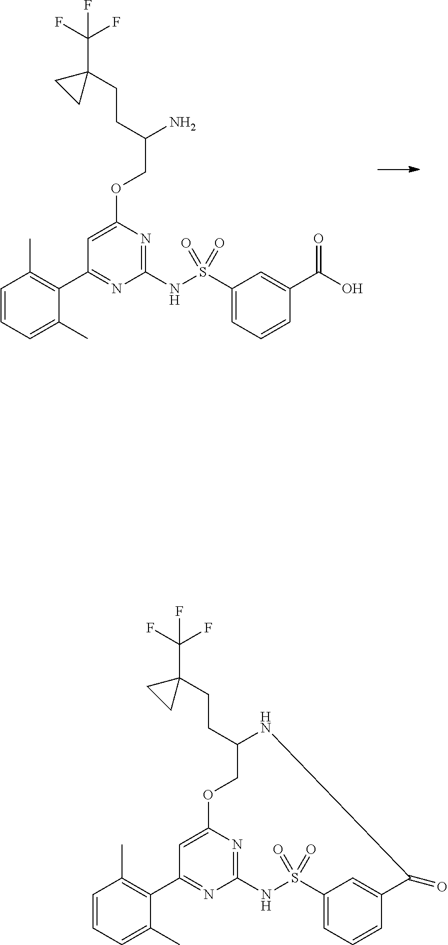 [SMILES: Cc1cccc(C)c1-c1cc(OCC(N)CCC2(C(F)(F)F)CC2)nc(NS(=O)(=O)c2cccc(C(=O)O)c2)n1 ; Cc1cccc(C)c1-c1cc2nc(n1)NS(=O)(=O)c1cccc(c1)C(=O)NC(CCC1(C(F)(F)F)CC1)CO2]

To a flask was added [dimethylamino(triazolo[4,5-b]pyridin-3-yloxy)methylene]-dimethyl-ammonium (Phosphorus Hexafluoride Ion) (93 mg, 0.2446 mmol), a solution of 3-[[4-[2-amino-4-[1-(trifluoromethyl)cyclopropyl]butoxy]-6-(2,6-dimethylphenyl)pyrimidin-2-yl]sulfamoyl]benzoic acid (hydrochloride salt) (120 mg, 0.1951 mmol) in DMF (1.5 mL), and Triethylamine (140 μL, 1.004 mmol). After 90 minutes, the reaction was filtered and purified via HPLC 25%-75% ACN:water with a 0.1% HCl modifier to give 6-(2,6-dimethylphenyl)-2,2-dioxo-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one (57.9 mg, 53%) as a white solid. ESI-MS m/z calc. 560.17053, found 561.1 (M+1)⁺; Retention time: 1.61 minutes, LC method A.

### Example 87: Preparation of Compound 93 and Compound 94

### Step 1: 3-[[4-[2-(tert-Butoxycarbonylamino)-4,4,4-trifluoro-butoxy]-6-(2,6-dimethylphenyl)pyrimidin-2-yl]sulfamoyl]benzoic Acid

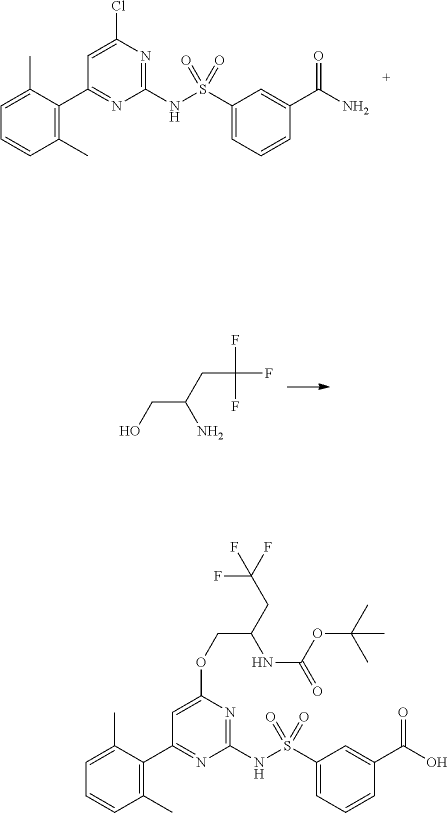 [SMILES: Cc1cccc(C)c1-c1cc(Cl)nc(NS(=O)(=O)c2cccc(C(N)=O)c2)n1 ; NC(CO)CC(F)(F)F ; Cc1cccc(C)c1-c1cc(OCC(CC(F)(F)F)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (0.63 g, 1.508 mmol), 2-amino-4,4,4-trifluoro-butan-1-ol (hydrochloride salt) (0.54 g, 3.007 mmol), and sodium t-butoxide (0.73 g, 7.596 mmol) in THF (8 mL) was stirred for five minutes, turning bright yellow. The reaction was placed in a preheated 60° C. bath and stirred for 25 minutes. UPLCMS showed complete conversion to amino intermediate. After cooling to room temperature, di-tert-butyl dicarbonate (0.67 g, 3.070 mmol) was added, and the reaction was stirred for 17 hours. The reaction was quenched with 1 M hydrochloric acid, diluted with water, and extracted with ethyl acetate. The combined extracts were washed with water, dried over sodium sulfate, and evaporated under vacuum. The residue was purified by silica gel column chromatography with 0-10% methanol in dichloromethane to give a mixture containing product. The mixture was re-purified by silica gel column chromatography with 0-9% methanol in dichloromethane to give 3-[[4-[2-(tert-butoxycarbonylamino)-4,4,4-trifluoro-butoxy]-6-(2,6-dimethylphenyl)pyrimidin-2-yl]sulfamoyl]benzoic acid (0.54 g, 57%) ESI-MS m/z calc. 624.1866, found 625.3 (M+1)⁺; Retention time: 0.67 minutes as a colorless solid, LC method D.

### Step 2: 3-[[4-(2-Amino-4,4,4-trifluoro-butoxy)-6-(2,6-dimethylphenyl)pyrimidin-2-yl]sulfamoyl]benzoic Acid

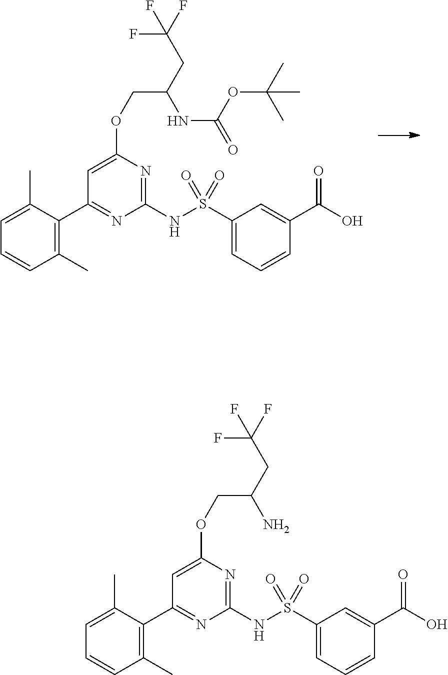 [SMILES: Cc1cccc(C)c1-c1cc(OCC(CC(F)(F)F)NC(=O)OC(C)(C)C)nc(NS(=O)(=O)c2cccc(C(=O)O)c2)n1 ; Cc1cccc(C)c1-c1cc(OCC(N)CC(F)(F)F)nc(NS(=O)(=O)c2cccc(C(=O)O)c2)n1]

A solution of 3-[[4-[2-(tert-butoxycarbonylamino)-4,4,4-trifluoro-butoxy]-6-(2,6-dimethylphenyl)pyrimidin-2-yl]sulfamoyl]benzoic acid (83 mg, 0.1329 mmol) and HCl (4 mL of 4 M, 16.00 mmol) (in dioxane) was stirred for one hour. The solvent was removed under vacuum, and the solids were triturated with diethyl ether to give 3-[[4-(2-amino-4,4,4-trifluoro-butoxy)-6-(2,6-dimethylphenyl)pyrimidin-2-yl]sulfamoyl]benzoic acid (hydrochloride salt) (81 mg, 109%) ESI-MS m/z calc. 524.13416, found 525.2 (M+1)⁺; Retention time: 0.39 minutes as a colorless solid, LC method D.

### Step 3: 6-(2,6-dimethylphenyl)-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione, enantiomer 1 (Compound 93), and 6-(2,6-dimethylphenyl)-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione, enantiomer 2 (Compound 94)

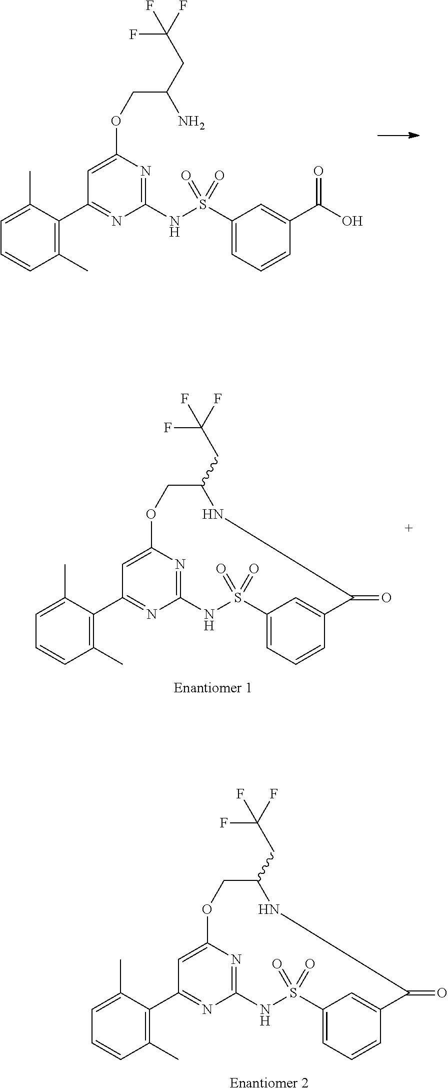 [SMILES: Cc1cccc(C)c1-c1cc(OCC(N)CC(F)(F)F)nc(NS(=O)(=O)c2cccc(C(=O)O)c2)n1 ; Cc1cccc(C)c1-c1cc2nc(n1)NS(=O)(=O)c1cccc(c1)C(=O)NC(CC(F)(F)F)CO2 ; Cc1cccc(C)c1-c1cc2nc(n1)NS(=O)(=O)c1cccc(c1)C(=O)NC(CC(F)(F)F)CO2]

A solution of 3-[[4-(2-amino-4,4,4-trifluoro-butoxy)-6-(2,6-dimethylphenyl)pyrimidin-2-yl]sulfamoyl]benzoic acid (hydrochloride salt) (45 mg, 0.08022 mmol), HATU (46 mg, 0.1210 mmol), and triethylamine (45 μL, 0.3229 mmol) in DMF (4 mL) was stirred for 17 hours. The reaction was diluted with water and extracted with ethyl acetate. The combined extracts were washed with brine and water, dried over sodium sulfate, and evaporated under vacuum. The residue was purified by reverse-phase HPLC-MS (1%-99% acetonitrile/water (5 mM HCl)) to give 20 mg of a mixture of enantiomers. The mixture was subjected to normal phase SFC-MS method using a (R,R)-Whelk-O column (150×2.1 mm, 3.5 m particle size) sold by Regis Technologies (pn: 780230), and a gradient run from 5-80% mobile phase B over 17.5 minutes. Mobile phase A=CO₂. Mobile phase B=MeOH (20 mM NH3). Flow rate=40 mL/min [20 mM NH3], and column temperature=55° C. to give enanantiomer 1, 6-(2,6-dimethylphenyl)-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione (7.8 mg, 38%) ESI-MS m/z calc. 506.12357, found 507.2 (M+1)⁺; Retention time: 1.29 minutes (LC method A), and enantiomer 2, 6-(2,6-dimethylphenyl)-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione (6.3 mg, 31%) ESI-MS m/z calc. 506.12357, found 507.2 (M+1)⁺; Retention time: 1.29 minutes (LC method A), both obtained as colorless solids.

### Example 88: Preparation of Compound 95

### Step 1: 3-[[4-[(2R)-2-(tert-Butoxycarbonylamino)propoxy]-6-(2,6-dimethylphenyl)pyrimidin-2-yl]sulfamoyl]benzoic Acid

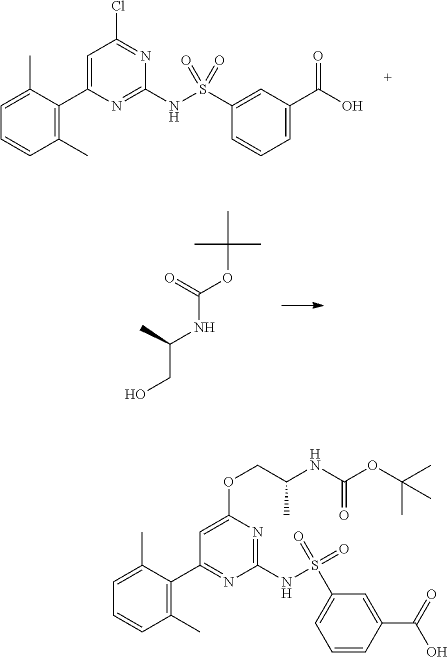 [SMILES: Cc1cccc(C)c1-c1cc(Cl)nc(NS(=O)(=O)c2cccc(C(=O)O)c2)n1 ; C[C@H](CO)NC(=O)OC(C)(C)C ; Cc1cccc(C)c1-c1cc(OC[C@@H](C)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (75 mg, 0.1795 mmol) in THE (0.7 mL) was added to tert-butyl N-[(1R)-2-hydroxy-1-methyl-ethyl]carbamate (approximately 47.17 mg, 0.2692 mmol). Solid sodium tert-butoxide (approximately 86.25 mg, 0.8975 mmol) was added after. The reaction mixture was allowed to stir overnight at room temperature. acetic acid (approximately 64.68 mg, 61.25 μL, 1.077 mmol) was added. The reaction mixture was diluted with DCM and washed with HCl (1 M, 1×7 mL) and brine (2×75 mL). The organic layer was dried over sodium sulfate, filtered and concentrated under reduced pressure. The crude product was chromatographed on a 12 gram silica gel column eluting with a EtOAc/hexane graidient. 3-[[4-[(2R)-2-(tert-Butoxycarbonylamino)propoxy]-6-(2,6-dimethylphenyl)pyrimidin-2-yl]sulfamoyl]benzoic Acid (1.65 g, 2.964 mmol) (65 mg, 65%) was obtained. ESI-MS m/z calc. 556.19916, found 557.3 (M+1)⁺; Retention time: 1.63 minutes; LC method A.

### Step 2: 3-[[4-[(2R)-2-Aminopropoxy]-6-(2,6-dimethylphenyl)pyrimidin-2-yl]sulfamoyl]benzoic Acid

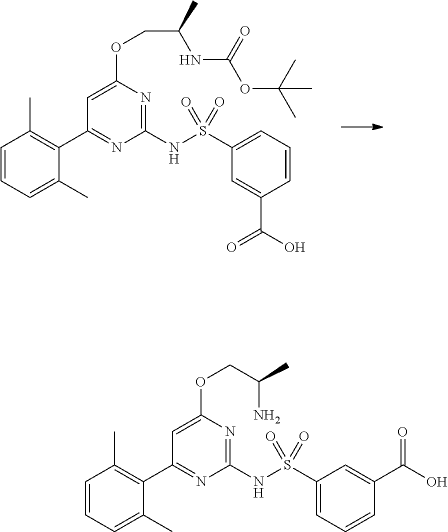 [SMILES: Cc1cccc(C)c1-c1cc(OC[C@@H](C)NC(=O)OC(C)(C)C)nc(NS(=O)(=O)c2cccc(C(=O)O)c2)n1 ; Cc1cccc(C)c1-c1cc(OC[C@@H](C)N)nc(NS(=O)(=O)c2cccc(C(=O)O)c2)n1]

A solution of 3-[[4-[(2R)-2-(tert-butoxycarbonylamino)propoxy]-6-(2,6-dimethylphenyl)pyrimidin-2-yl]sulfamoyl]benzoic acid (1.65 g, 2.964 mmol) in HCl (8 mL of 4 M, 32.00 mmol) (in dioxane) was stirred for two hours, and the solvent was removed under vacuum. The solids were triturated with diethyl ether and dried under vacuum to give 3-[[4-[(2R)-2-aminopropoxy]-6-(2,6-dimethylphenyl)pyrimidin-2-yl]sulfamoyl]benzoic Acid (hydrochloride salt) (1.55 g, 106%) as a colorless solid. ESI-MS m/z calc. 456.14673, found 457.2 (M+1)⁺; Retention time: 0.37 minutes, LC method D.

### Step 3: (11R)-6-(2,6-Dimethylphenyl)-11-methyl-9-oxa-2λ⁶-thia-3,5,12,19-tetraazatricyclo[12.3.1.14,8]nonadeca-1(17),4,6,8(19),14(18),15-hexaene-2,2,13-trione (Compound 95)

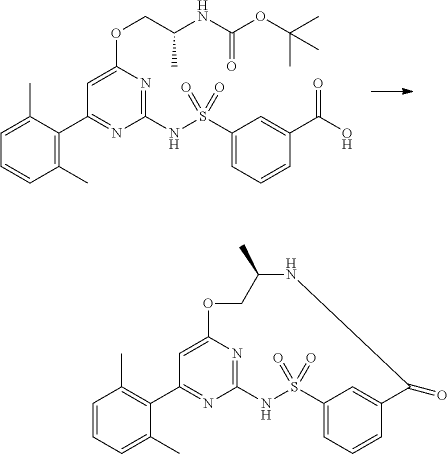 [SMILES: Cc1cccc(C)c1-c1cc(OC[C@@H](C)NC(=O)OC(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)N[C@H](C)CO2]

3-[[4-[(2R)-2-(tert-Butoxycarbonylamino)propoxy]-6-(2,6-dimethylphenyl)pyrimidin-2-yl]sulfamoyl]benzoic acid (1.65 g, 2.964 mmol) was dissolved in HATU in dioxane. After stirring at room temperature for 1 hour, volatiles were removed under reduced pressure. The remaining material was dissolved in DMF (0.7 mL). triethylamine was added. After stirring at room temperature for 15 minutes, the product was isolated by UV-triggered reverse-phase HPLC using a Luna C₁₈(2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. to give (11R)-6-(2,6-dimethylphenyl)-11-methyl-9-oxa-2λ⁶-thia-3,5,12,19-tetraazatricyclo[12.3.1.14,8]nonadeca-1(17),4,6,8(19),14(18),15-hexaene-2,2,13-trione (19.2 mg). ESI-MS m/z calc. 438.13617, found 439.2 (M+1)⁺; Retention time: 1.22 minutes; LC method A.

### Example 89: Preparation of Compound 96

### Step 1: (11R)-6-(2,6-Dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 96)

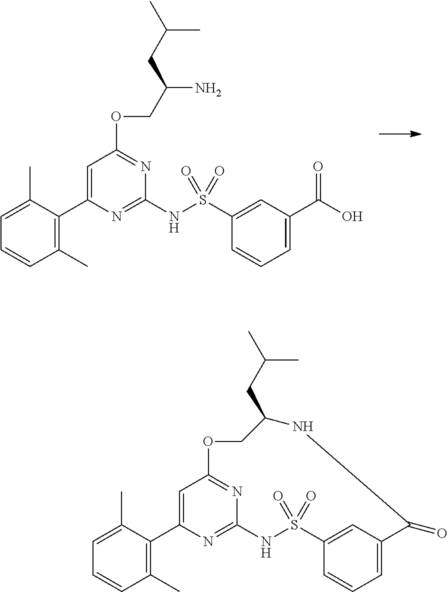 [SMILES: Cc1cccc(C)c1-c1cc(OC[C@H](N)CC(C)C)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CC(C)C)CO2]

3-[[4-[(2R)-2-amino-4-methyl-pentoxy]-6-(2,6-dimethylphenyl)pyrimidin-2-yl]sulfamoyl]benzoic acid (hydrochloride salt) (17 mg, 0.03177 mmol) and triethylamine (20 μL, 0.1435 mmol) were dissolved in DMF (1 mL) and HATU (14 mg, 0.03682 mmol) was added. The reaction mixture was stirred for 3 h at room temperature. The reaction mixture was filtered and purified by LC/MS utilizing a gradient of 1-99% acetonitrile in 5 mM aqueous HCl to yield (11R)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (9 mg, 57%). ESI-MS m/z calc. 480.18314, found 481.4 (M+1)⁺; Retention time: 1.67 minutes; LC method A.

### Example 90: Preparation of Compound 97

### Step 1: (11R)-6-(2,6-Dimethylphenyl)-11-(2,2-dimethylpropyl)-2,2-dioxo-9-oxa-2)⁶-thia-3,5,12,19-tetrazatricyclo[12.3.1.14,8]nonadeca-1(18),4(19),5,7,14,16-hexaen-13-one (Compound 97)

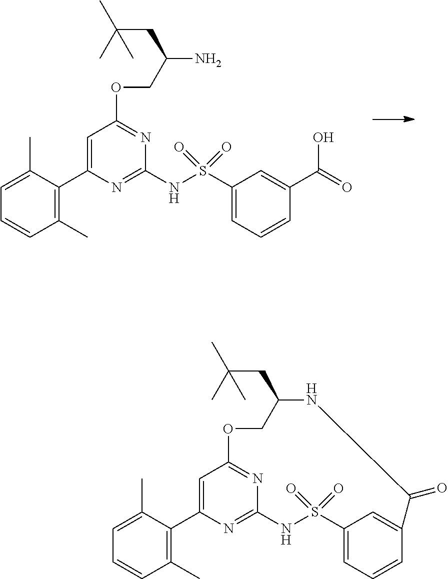 [SMILES: Cc1cccc(C)c1-c1cc(OC[C@H](N)CC(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CC(C)(C)C)CO2]

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (4 g, 7.285 mmol) in EtOAc (200 mL) and DMF (50 mL) were added TEA (5 mL, 35.87 mmol) and an EtOAc solution of T3P (10 mL of 50% w/w, 16.80 mmol). The reaction mixture was stirred at room temperature for 3 h. The resulting mixture was diluted with water (300 mL) and the product was extracted with EtOAc (3×300 mL). The combined organic layers were washed with 10% brine (300 mL), dried with NaSO₄, filtered and evaporated to dryness. (11R)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (2.63 g, 73%) was used without further purification. ESI-MS m/z calc. 494.19876, found 495.108 (M+1)⁺; Retention time: 0.58 minutes; (LC method A). ¹H NMR (400 MHz, DMSO-d₆) δ 8.53 (s, 1H), 7.95-7.89 (m, 3H), 7.68 (d, J 10.7 Hz, 2H), 7.25 (t, J 7.6 Hz, 1H), 7.12 (d, J 7.7 Hz, 2H), 6.42 (s, 1H), 5.11 (dd, J 10.8, 4.0 Hz, 1H), 4.03 (q, J 7.1 Hz, 1H), 3.84 (t, J 11.1 Hz, 1H), 2.89 (s, 1H), 2.73 (s, 1H), 1.52 (dd, J 14.6, 8.6 Hz, 1H), 1.43 (d, J 14.4 Hz, 1H), 1.17 (t, J 7.1 Hz, 2H), 0.55 (s, 9H).

### Example 91: Preparation of Compound 98

### Step 1: (11R)-6-(2-Fluoro-6-methylphenyl)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 98)

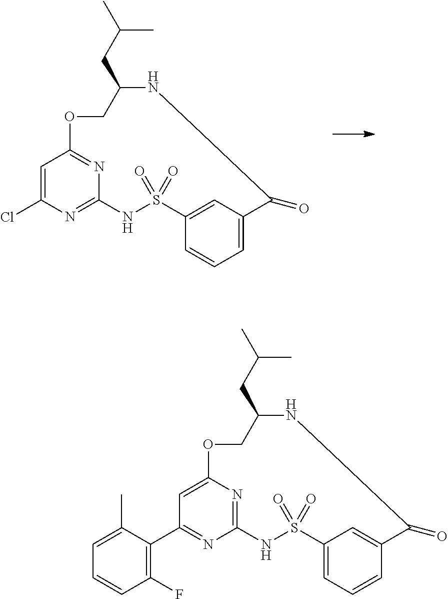 [SMILES: CC(C)C[C@@H]1COc2cc(Cl)nc(n2)NS(=O)(=O)c2cccc(c2)C(=O)N1 ; Cc1cccc(F)c1-c1cc2nc(n1)NS(=O)(=O)c1cccc(c1)C(=O)N[C@H](CC(C)C)CO2]

(2-Fluoro-6-methyl-phenyl)boronic acid (approximately 26.23 mg, 0.1704 mmol) and (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (50 mg, 0.1217 mmol) were combined and dissolved in DMSO (0.5 mL). Aqueous potassium carbonate (approximately 182.5 μL of 2 M, 0.3651 mmol) was added followed by Pd(dppf)Cl₂ (approximately 4.969 mg, 0.006085 mmol). The reaction mixture was capped under nitrogen gas and allowed to stir at 120° C. for 45 minutes. The reaction mixture was filtered and purified by UV-triggered reverse-phase HPLC: Samples were purified using a reverse phase HPLC method using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-6-(2-fluoro-6-methylphenyl)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (35.4 mg, 60%) was obtained. ESI-MS m/z calc. 484.15805, found 485.3 (M+1)⁺; Retention time: 1.49 minutes; LC method A.

### Example 92: Preparation of Compound 99

### Step 1: (11R)-6-Chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one

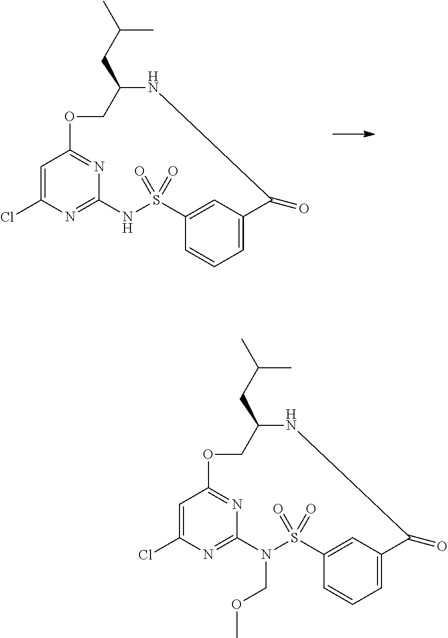 [SMILES: CC(C)C[C@@H]1COc2cc(Cl)nc(n2)NS(=O)(=O)c2cccc(c2)C(=O)N1 ; COCN1c2nc(Cl)cc(n2)OC[C@@H](CC(C)C)NC(=O)c2cccc(c2)S1(=O)=O]

To a solution of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (3 g, 7.301 mmol) in DCM (10.00 mL) was added potassium carbonate (1.6 g, 11.58 mmol) and chloro(methoxy)methane (750 μL, 9.874 mmol) and the mixture was stirred at rt for 18 h. A 1:1 mixture of saturated ammonium chloride and brine solution was added and it was extracted with ethyl acetate. The organics were separated, dried over sodium sulfate. The resultant residue was purified by silica gel column chromatography using a shallow gradient 100% hexanes to 100% EtOAc to afford (11R)-6-chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one (2.9 g, 87%) as white solid. ESI-MS m/z calc. 454.10776, found 455.3 (M+1)⁺; Retention time: 0.68 minutes; LC method D.

### Step 2: (11R)-6-(2,6-Diisopropylphenyl)-11-isobutyl-2,2-dioxo-9-oxa-2⁶-thia-3,5,12,19-tetrazatricyclo[12.3.1.14,8]nonadeca-1(18),4,6,8(19),14,16-hexaen-13-one (Compound 99)

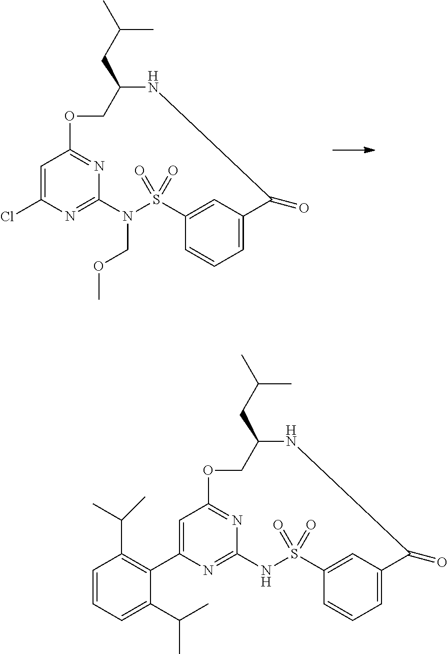 [SMILES: COCN1c2nc(Cl)cc(n2)OC[C@@H](CC(C)C)NC(=O)c2cccc(c2)S1(=O)=O ; CC(C)C[C@@H]1COc2cc(-c3c(C(C)C)cccc3C(C)C)nc(n2)NS(=O)(=O)c2cccc(c2)C(=O)N1]

To a solution of (11R)-6-chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one (25 mg, 0.05495 mmol) in DMA (2 mL) in a microwave vial was added (2,6-diisopropylphenyl)boronic acid (21 mg, 0.1019 mmol) and the solution was purged with a continuous stream of nitrogen for 5 min. Pd(dppf)Cl₂ (12 mg, 0.01469 mmol) and potassium carbonate (200 μL of 2 M, 0.4000 mmol) was added and the orange color solution was purged with a continuous stream of nitrogen for 5 min. The vial was capped and heated at 100° C. for 1 h. The reaction mixture was cooled to rt and diluted with ethyl acetate and filtered through Celite along with a short plug of silica gel. The filtrate was concentrated and dissolved in 1 mL of 1:4 TFA-DCM premixed solution TFA (100 μL, 1.298 mmol) and DCM (400 μL) and stirred at rt overnight. Solvent was removed and the residue was dissolved in 2 mL of DMSO. It was filtered and purified by reverse phase HPLC-MS using a dual gradient run from 30-99% mobile phase B over 15.0 minutes. Mobile phase A=H₂O (5 mM HCl). Mobile phase B=CH₃CN to afford (11R)-6-(2,6-diisopropylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (18 mg, 58%) as white solid. ESI-MS m/z calc. 536.2457, found 537.11 (M+1)⁺; Retention time: 1.4 minutes; LC method Q. ¹H NMR (500 MHz, DMSO-d₆) δ 13.19 (s, 1H), 8.51 (s, 1H), 7.92 (d, J=14.1 Hz, 2H), 7.69 (s, 2H), 7.43 (s, 1H), 7.31-7.17 (m, 2H), 6.46 (s, 1H), 5.15 (d, J=10.8 Hz, 1H), 3.90 (t, J 11.1 Hz, 1H), 3.38 (s, 2H), 2.56 (q, J 6.8 Hz, 1H), 2.19 (d, J 7.2 Hz, 1H), 1.56-1.40 (m, 2H), 1.19 (s, 3H), 1.10 (d, J 6.8 Hz, 3H), 1.03 (s, 3H), 0.98 (d, J 6.7 Hz, 3H), 0.74 (d, J 6.4 Hz, 3H), 0.27-0.20 (m, 3H).

### Example 93: Preparation of Compound 100

### Step 1: (11R)-6-(2,6-Dimethylcyclohex-1-en-1-yl)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 100)

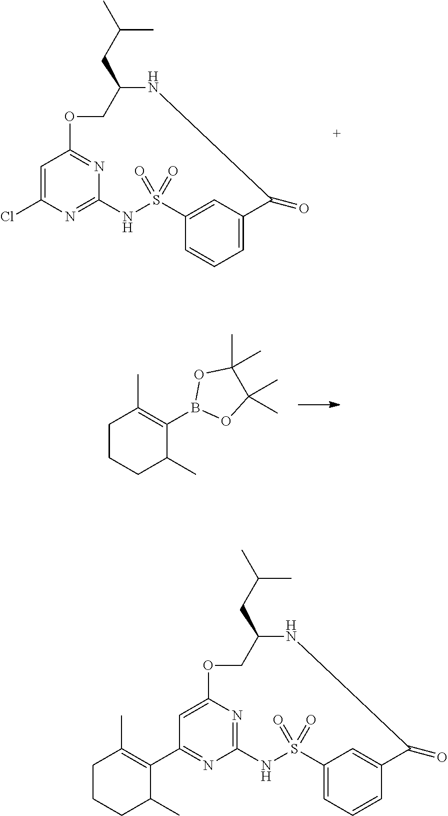 [SMILES: CC(C)C[C@@H]1COc2cc(Cl)nc(n2)NS(=O)(=O)c2cccc(c2)C(=O)N1 ; CC1=C(B2OC(C)(C)C(C)(C)O2)C(C)CCC1 ; CC1=C(c2cc3nc(n2)NS(=O)(=O)c2cccc(c2)C(=O)N[C@H](CC(C)C)CO3)C(C)CCC1]

(11R)-6-Chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (30 mg, 0.07301 mmol) and 2-(2,6-dimethylcyclohexen-1-yl)-4,4,5,5-tetramethyl-1,3,2-dioxaborolane (approximately 18.97 mg, 0.08031 mmol) were combined and dissolved in DMSO (0.5 mL). Aqueous potassium carbonate (approximately 109.5 μL of 2 M, 0.2190 mmol) was added followed by [1,1′-bis(diphenylphosphino)ferrocene]dichloropalladium(II) (approximately 2.671 mg, 0.003650 mmol). The reaction mixture was capped under nitrogen gas and stirred at 120° C. for 40 minutes. After filtration, the product was purified by reverse phase HPLC using a Luna Cis (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-6-(2,6-dimethylcyclohex-1-en-1-yl)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (18 mg, 51%) was obtained (2.3 mg, 51%). ESI-MS m/z calc. 484.21442, found 485.4 (M+1)⁺; Retention time: 1.64 minutes; LC method A.

### Example 94: Preparation of Compound 101

### Step 1: (11R)-11-Isobutyl-2,2-dioxo-6-(2-phenylphenyl)-9-oxa-2λ⁶-thia-3,5,12,19-tetrazatricyclo[12.3.1.14,8]nonadeca-1(18),4,6,8(19),14,16-hexaen-13-one (Compound 101)

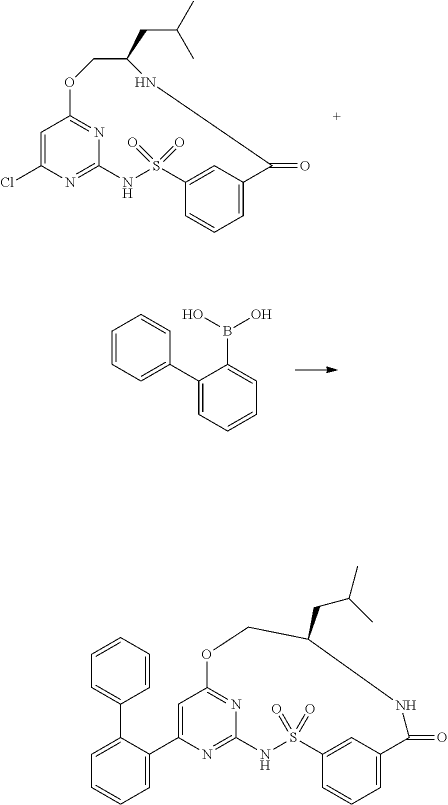 [SMILES: CC(C)C[C@@H]1COc2cc(Cl)nc(n2)NS(=O)(=O)c2cccc(c2)C(=O)N1 ; OB(O)c1ccccc1-c1ccccc1 ; CC(C)C[C@@H]1COc2cc(-c3ccccc3-c3ccccc3)nc(n2)NS(=O)(=O)c2cccc(c2)C(=O)N1]

A heterogeneous mixture of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (50 mg, 0.1217 mmol), 1,3-bis(2,6-diisopropylphenyl)-4,5-dihydroimidazole; 3-chloropyridine; dichloropalladium (42 mg, 0.06154 mmol), (2-phenylphenyl)boronic acid (75 mg, 0.3787 mmol), and potassium tert-butoxide (55 mg, 0.4901 mmol) in tert-butanol (750 μL) was microwaved for 3 h at 100° C. in a sealed microwave tube. The mixture was cooled to ambient temperature and then the mixture was filtered and concentrated under a stream of nitrogen to give a residue. This mixture was purified by reverse-phase preparative chromatography utilizing a C₁₈ column and a 20-80% gradient of acetonitrile in water containing 5 mM HCl to afford as a tan solid (11R)-11-isobutyl-2,2-dioxo-6-(2-phenylphenyl)-9-oxa-2λ⁶-thia-3,5,12,19-tetrazatricyclo[12.3.1.14,8]nonadeca-1(18),4,6,8(19),14,16-hexaen-13-one (37.50 mg, 57%). ESI-MS m/z calc. 528.1831, found 529.3 (M+1)⁺; Retention time: 1.64 minutes; LC method A. ¹H NMR (500 MHz, DMSO-d₆) δ 12.59 (s, 1H), 8.45 (s, 1H), 7.98-7.88 (m, 1H), 7.84 (d, J 9.9 Hz, 1H), 7.68 (d, J 4.8 Hz, 2H), 7.62-7.54 (m, 2H), 7.49 (t, J 7.5 Hz, 1H), 7.43 (d, J 7.6 Hz, 1H), 7.31-7.24 (m, 3H), 7.13 (dd, J 6.5, 3.0 Hz, 2H), 5.85 (s, 1H), 5.02 (dd, J 11.0, 3.8 Hz, 1H), 3.72 (t, J=11.1 Hz, 1H), 3.16 (tt, J 11.6, 5.6 Hz, 1H), 1.48 (ddt, J 13.5, 6.8, 3.8 Hz, 1H), 1.40 (ddd, J 14.3, 11.0, 3.4 Hz, 1H), 0.93 (t, J 11.9 Hz, 1H), 0.76 (d, J 6.7 Hz, 3H), 0.25 (d, J 6.5 Hz, 3H).

### Example 95: Preparation of Compound 102

### Step 1: (11R)-11-(2-Methylpropyl)-6-[1-(2-methylpropyl)-1H-pyrazol-5-yl]-9-oxa-2λ⁶-thia-3,5,12,19-tetraazatricyclo[12.3.1.14,8]nonadeca-1(17),4(19),5,7,14(18),15-hexaene-2,2,13-trione (Compound 102)

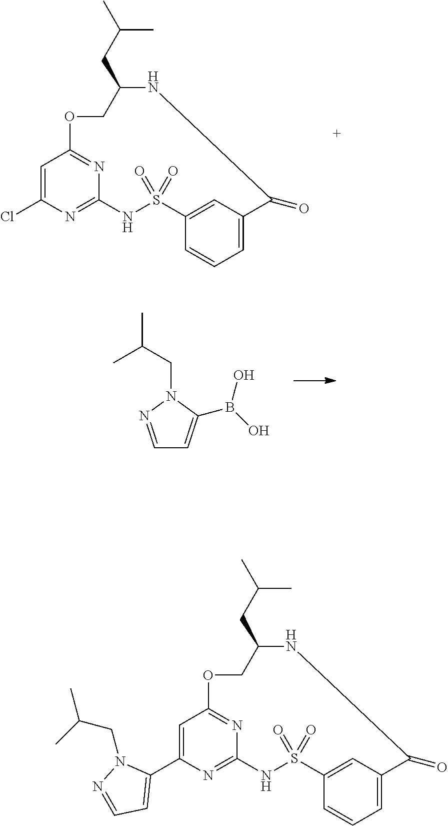 [SMILES: CC(C)C[C@@H]1COc2cc(Cl)nc(n2)NS(=O)(=O)c2cccc(c2)C(=O)N1 ; CC(C)Cn1nccc1B(O)O ; CC(C)C[C@@H]1COc2cc(-c3ccnn3CC(C)C)nc(n2)NS(=O)(=O)c2cccc(c2)C(=O)N1]

A solution of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (25 mg, 0.06085 mmol) in dioxane (0.5 mL) was added to (2-isobutylpyrazol-3-yl)boronic acid (approximately 12.27 mg, 0.07302 mmol). Aqueous potassium carbonate (approximately 60.85 μL of 2 M, 0.1217 mmol) was added. tetrakis(triphenylphosphane)palladium(0) (approximately 3.515 mg, 0.003042 mmol) was added under nitrogen. The reaction vial was capped under nitrogen and stirred at 120° C. for 45 minutes. After cooling to room temperature, the reaction mixture was filtered and purified by reverse phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-11-(2-methylpropyl)-6-[1-(2-methylpropyl)-1H-pyrazol-5-yl]-9-oxa-2λ⁶-thia-3,5,12,19-tetraazatricyclo[12.3.1.14,8]nonadeca-1(17),4(19),5,7,14(18),15-hexaene-2,2,13-trione (15.8 mg, 52%) was obtained. ESI-MS m/z calc. 498.20493, found 499.1 (M+1)⁺; Retention time: 1.56 minutes; LC method A.

### Example 96: Preparation of Compound 103

### Step 1: (11R)-11-(2-Methylpropyl)-6-[2-(trifluoromethoxy)phenyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 103)

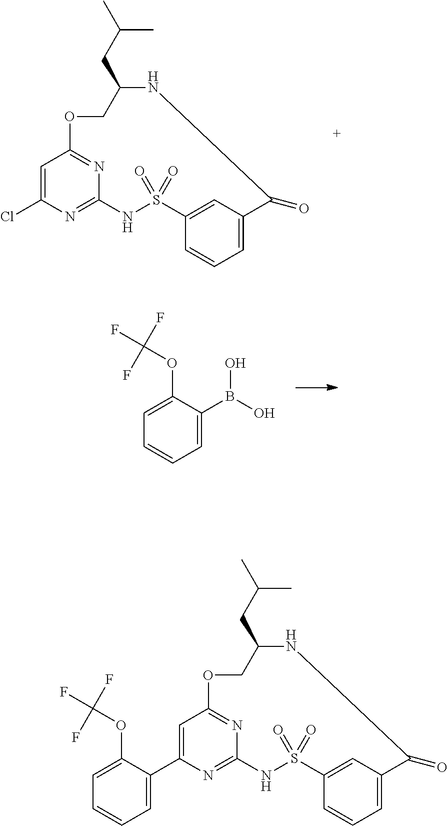 [SMILES: CC(C)C[C@@H]1COc2cc(Cl)nc(n2)NS(=O)(=O)c2cccc(c2)C(=O)N1 ; OB(O)c1ccccc1OC(F)(F)F ; CC(C)C[C@@H]1COc2cc(-c3ccccc3OC(F)(F)F)nc(n2)NS(=O)(=O)c2cccc(c2)C(=O)N1]

A solution of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (25 mg, 0.06085 mmol) in dioxane (0.5 mL) was added to [2-(trifluoromethoxy)phenyl]boronic acid (approximately 15.04 mg, 0.07302 mmol). Aqueous potassium carbonate (approximately 60.85 μL of 2 M, 0.1217 mmol) was added. tetrakis(triphenylphosphane)palladium(0) (approximately 3.515 mg, 0.003042 mmol) was added under nitrogen. The reaction vial was capped under nitrogen and stirred at 120° C. for 45 minutes. After cooling to room temperature, the reaction mixture was filtered and purified by reverse phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-11-(2-methylpropyl)-6-[2-(trifluoromethoxy)phenyl]-9-oxa-2λ⁶-thia-3,5,12,19-tetraazatricyclo[12.3.1.14,8]nonadeca-1(17),4(19),5,7,14(18),15-hexaene-2,2,13-trione (3.9 mg, 12%) was obtained. ESI-MS m/z calc. 536.13416, found 537.2 (M+1)⁺; Retention time: 1.7 minutes; LC method A.

### Example 97: Preparation of Compound 104

### Step 1: 1-Bromo-2-(3-methyl-butyl)-benzene

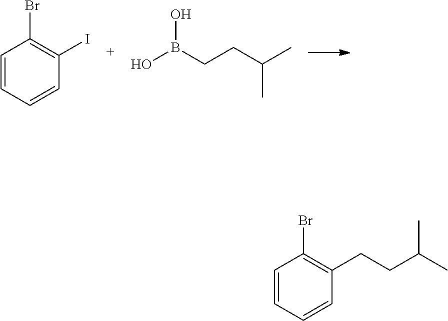

A mixture of 1-bromo-2-iodobenzene (11.0 g, 38.87 mmol), 3-methylbutylboronic acid (4.96 g, 42.76 mmol), potassium phosphate (16.50 g, 77.74 mmol) and 1,1′-bis(diphenylphosphino)ferrocene-palladium(II)dichloride dichloromethane complex (1.59 g, 1.94 mmol) in anhydrous tetrahydrofuran (155 mL) was bubbled with argon for 10 minutes, then sealed and stirred at 90° C. for 19 hours. Water (200 mL) and diethyl ether (300 mL) were added. The solution was filtered, and the organic layer was separated. The aqueous layer was extracted with ether (2×300 mL) and the combined organic layer was washed with brine (2×100 mL), dried over magnesium sulfate and concentrated. The residue was purified by silica gel column chromatography using hexane to afford the crude prude product, which was further purified by reverse phase column chromatography using 0-100% acetonitrile-water (0.1% trifluoroacetic acid). The pure fractions were combined and concentrated under the reduced pressure to remove most of acetonitrile, then extracted with diethyl ether (3×100 mL). The combined organics was dried over magnesium sulfate and concentrated to afford 1-bromo-2-(3-methyl-butyl)-benzene (4.12 g, 47%) as a colorless oil. ¹H NMR (250 MHz, CDCl₃) δ (ppm): 7.53 (d, J 8.0 Hz, 1H), 7.23 (m, 2H), 7.05 (m, 1H), 2.74 (m, 2H), 1.64 (m, 1H), 1.51 (m, 2H), 0.98 (d, J=6.5 Hz, 6H).

### Step 2: 2-(2-Isopentylphenyl)-4,4,5,5-tetramethyl-1,3,2-dioxaborolane

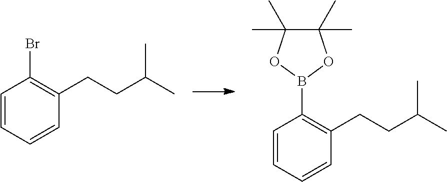 [SMILES: CC(C)CCc1ccccc1Br ; CC(C)CCc1ccccc1B1OC(C)(C)C(C)(C)O1]

Argon gas was bubbled through a mixture of 1-bromo-2-(3-methyl-butyl)-benzene (4.11 g, 18.10 mmol), 4,4,5,5-tetramethyl-2-(4,4,5,5-tetramethyl-1,3,2-dioxaborolan-2-yl)-1,3,2-dioxaborolane (5.05 g, 19.91 mmol), potassium acetate (5.33 g, 54.30 mmol) and 1,1′-bis(diphenylphosphino)ferrocene-palladium(II)dichloride dichloromethane complex (739 mg, 0.91 mmol) in anhydrous dioxane (90 mL) for 10 minutes. The reaction vessel was sealed, and the reaction mixture was stirred at 80° C. for 20 hours. Diethyl ether (200 mL) and water (100 mL) were added. The organic layer was separated, and the aqueous layer was extracted with diethyl ether (2×200 mL). The combined organic layer was washed with brine (2×50 mL), dried over magnesium sulfate and concentrated. The residue obtained was purified by silica gel column chromatography using 0-10% hexanes-dichloromethane to afford 2-(2-isopentylphenyl)-4,4,5,5-tetramethyl-1,3,2-dioxaborolane (3.22 g, 65%) as a pale yellow liquid. ¹H NMR (250 MHz, DMSO) δ (ppm): 7.61 (m, 1H), 7.37 (m, 1H), 7.15 (m. 2H), 2.80 (m, 2H), 1.58 (m, 1H), 1.36 (m, 2H), 1.34 (s, 12H), 0.92 (d, J 6.5 Hz, 6H).

### Step 3: (11R)-6-[2-(3-Methylbutyl)phenyl]-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 104)

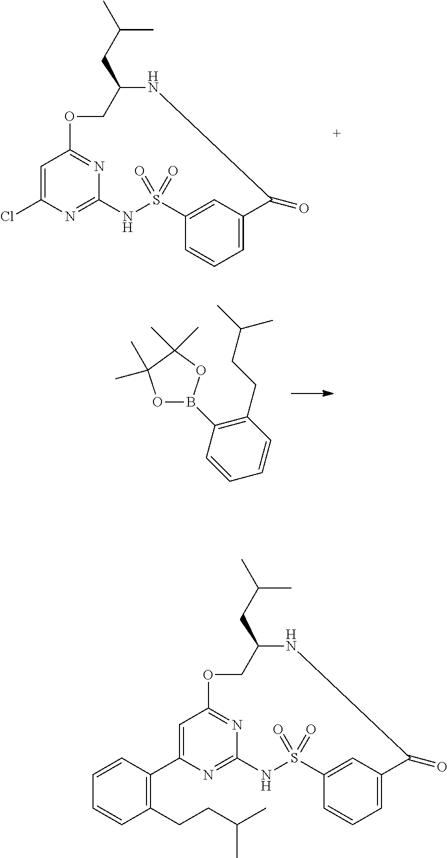

A solution of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (25 mg, 0.06085 mmol) in dioxane (0.5 mL) was added to 2-(2-isopentylphenyl)-4,4,5,5-tetramethyl-1,3,2-dioxaborolane (approximately 20.02 mg, 0.07302 mmol). Aqueous potassium carbonate (approximately 60.85 μL of 2 M, 0.1217 mmol) was added. tetrakis(triphenylphosphane)palladium(0) (approximately 3.515 mg, 0.003042 mmol) was added under nitrogen. The reaction vial was capped under nitrogen and stirred at 120° C. for 45 minutes. After cooling to room temperature, the reaction mixture was filtered and purified by reverse phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-6-[2-(3-methylbutyl)phenyl]-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (5 mg, 15%) was obtained. ESI-MS m/z calc. 522.2301, found 523.3 (M+1)⁺; Retention time: 1.87 minutes; LC method A.

### Example 98: Preparation of Compound 105

### Step 1: (11R)-6-(2-cyclopropylphenyl)-11-(2-methylpropyl)-9-oxa-2⁶-thia-3,5,12,19-tetraazatricyclo[12.3.1.14,8]nonadeca-1(17),4(19),5,7,14(18),15-hexaene-2,2,13-trione (Compound 105)

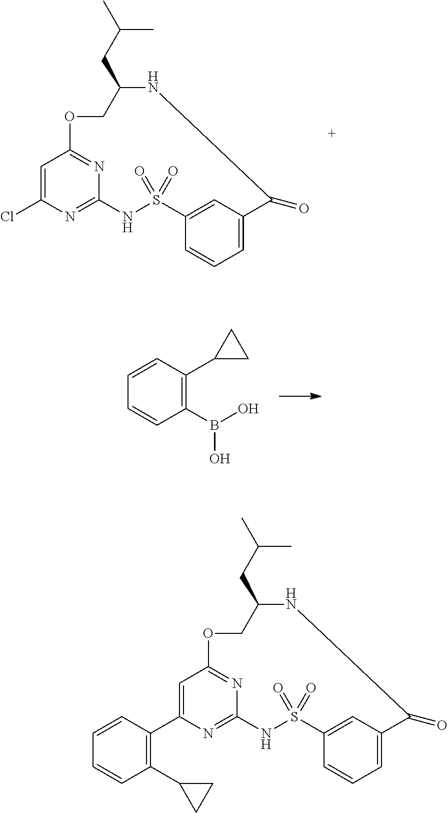 [SMILES: CC(C)C[C@@H]1COc2cc(Cl)nc(n2)NS(=O)(=O)c2cccc(c2)C(=O)N1 ; OB(O)c1ccccc1C1CC1 ; CC(C)C[C@@H]1COc2cc(-c3ccccc3C3CC3)nc(n2)NS(=O)(=O)c2cccc(c2)C(=O)N1]

A solution of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (25 mg, 0.06085 mmol) in dioxane (0.5 mL) was added to (2-cyclopropylphenyl)boronic acid (approximately 11.83 mg, 0.07302 mmol). Aqueous potassium carbonate (approximately 60.85 μL of 2 M, 0.1217 mmol) was added. tetrakis(triphenylphosphane)palladium(0) (approximately 3.515 mg, 0.003042 mmol) was added under nitrogen. The reaction vial was capped under nitrogen and stirred at 120° C. for 45 minutes. After cooling to room temperature, the reaction mixture was filtered and purified by reverse phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-6-(2-cyclopropylphenyl)-11-(2-methylpropyl)-9-oxa-2λ⁶-thia-3,5,12,19-tetraazatricyclo[12.3.1.14,8]nonadeca-1(17),4(19),5,7,14(18),15-hexaene-2,2,13-trione (10.5 mg, 35%) was obtained. ESI-MS m z calc. 492.18314, found 493.1 (M+1)⁺; Retention time: 1.55 minutes; LC method A.

### Example 99: Preparation of Compound 106

### Step 1: (2-Bromo-3-methyl-phenyl)methanol

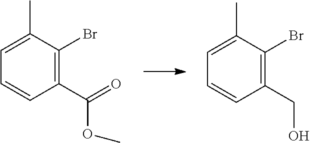

To a solution of methyl 2-bromo-3-methyl-benzoate (10.0281 g, 42.902 mmol) in anhydrous THE (100 mL) stirring at 0° C. was added Lithium Borohydride (4.9305 g, 215.02 mmol). The reaction mixture was then heated to and stirred at 50° C. for 4 h. The reaction was diluted with DI water (30 mL) and extracted with EtOAc (3×50 mL). The combined EtOAc layers were washed with saturated aqueous NaCl (100 mL), dried over anhydrous sodium sulfate, filtered and concentrated under vacuum. The crude product (8.637 g) was obtained as a light orange solid. (2-bromo-3-methyl-phenyl)methanol (8.637 g, 100%). H NMR (500 MHz, DMSO-d₆) δ 7.37 (d, J 7.5 Hz, 1H), 7.28 (t, J 7.5, 7.5 Hz, 1H), 7.23 (d, J 7.3 Hz, 1H), 5.39 (t, J 5.6, 5.6 Hz, 1H), 4.51 (d, J 5.7 Hz, 2H), 2.35 (s, 3H).

### Step 2: 1-(Benzyloxymethyl)-2-bromo-3-methyl-benzene

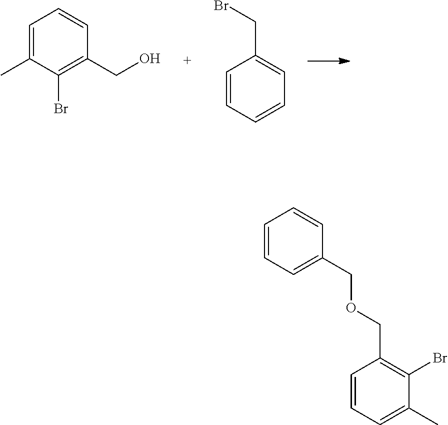

To (2-bromo-3-methyl-phenyl)methanol (1.87 g, 9.301 mmol) in DMSO (38 mL) cooled to 0° C. in an ice bath was added NaH (1.227 g of 60% w/w, 30.68 mmol) and the reaction was stirred for 15 minutes. Then bromomethylbenzene (1.75 mL, 14.71 mmol) was added and the mixture was allowed warm to rt and stir for 16 h. The mixture was partitioned between EtOAc and water. The organic layer was washed with brine, dried (sodium sulfate), filtered and concentrated to a solid which was purified by silica gel chromatography (80 gram column) using a shallow gradient from 100% hexanes to 40% EtOAc (compound elutes at 18% ethyl acetate) giving 1-(benzyloxymethyl)-2-bromo-3-methyl-benzene (2.69 g, 99%) ESI-MS m z calc. 290.03064, found 291.2 (M+1)⁺; Retention time: 2.06 minutes.

### Step 3: 2-[2-(Benzyloxymethyl)-6-methyl-phenyl]-4,4,5,5-tetramethyl-1,3,2-dioxaborolane

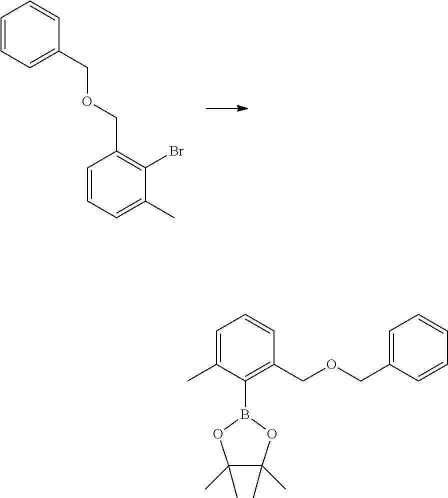 [SMILES: Cc1cccc(COCc2ccccc2)c1Br ; Cc1cccc(COCc2ccccc2)c1B1OC(C)(C)C(C)(C)O1]

In a 350 mL sealed vessel was dissolved 1-(benzyloxymethyl)-2-bromo-3-methyl-benzene (5.4 g, 18.55 mmol) in dioxane (55 mL) and to it was added KOAc (3.85 g, 39.23 mmol) and the mixture was degassed with nitrogen for several minutes. Then bis(pinacol)diboron (7.25 g, 28.55 mmol) was added, followed by Pd(dppf)Cl₂ (1.41 g, 1.932 mmol) and the reaction was purged again by N₂, sealed and heated to 100° C. for 16 hours. After the reaction was cooled to room temperature, saturated ammonium chloride was added, and the reaction was extracted with ethyl acetate. The combined organic extracts were washed with brine, dried over sodium sulfate, filtered and concentrated. The resulting brown oil was purified by silica gel column chromatography (220 gram column) using a gradient of 100% hexanes to 30% ethyl acetate in hexanes (compound elutes at 10% ethyl acetate) to obtain the desired compound as a white solid 2-[2-(benzyloxymethyl)-6-methyl-phenyl]-4,4,5,5-tetramethyl-1,3,2-dioxaborolane (4.63 g, 74%). ESI-MS m/z calc. 338.20532, found 339.4 (M+1)⁺; Retention time: 2.23 minutes; LC method A. ¹H NMR (499 MHz, Chloroform-d) δ 7.37-7.32 (m, 4H), 7.32-7.27 (m, 1H), 7.25-7.20 (m, 1H), 7.10 (dd, J 23.3, 7.5 Hz, 2H), 4.62 (s, 2H), 4.46 (s, 2H), 2.45 (s, 3H), 1.34 (s, 12H).

### Step 4: tert-Butyl N-[4-[2-(benzyloxymethyl)-6-methyl-phenyl]-6-chloro-pyrimidin-2-yl]-N-tert-butoxycarbonyl-carbamate

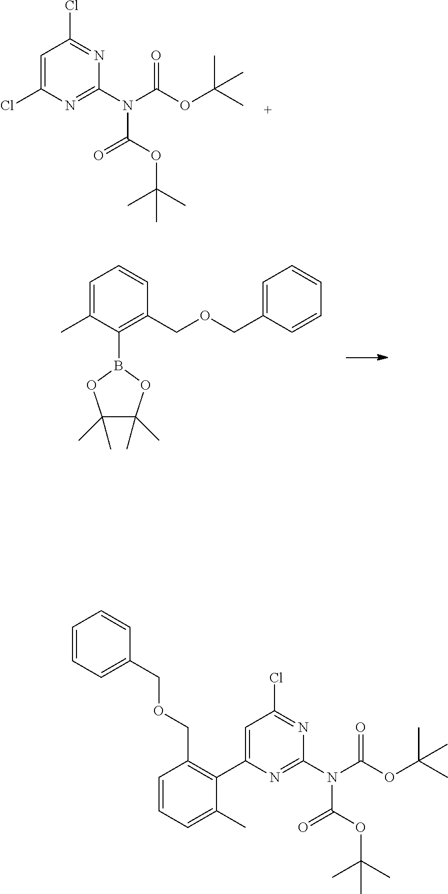 [SMILES: CC(C)(C)OC(=O)N(C(=O)OC(C)(C)C)c1nc(Cl)cc(Cl)n1 ; Cc1cccc(COCc2ccccc2)c1B1OC(C)(C)C(C)(C)O1 ; Cc1cccc(COCc2ccccc2)c1-c1cc(Cl)nc(N(C(=O)OC(C)(C)C)C(=O)OC(C)(C)C)n1]

tert-Butyl N-tert-butoxycarbonyl-N-(4,6-dichloropyrimidin-2-yl)carbamate (1.5 g, 4.118 mmol) and 2-[2-(benzyloxymethyl)-6-methyl-phenyl]-4,4,5,5-tetramethyl-1,3,2-dioxaborolane (1.4 g, 4.139 mmol) were combined in dimethoxyethane (36 mL) and water (6 mL). Added to the mixture were [1,1′-bis(diphenylphosphino)ferrocene]dichloropalladium(II) (315 mg, 0.4305 mmol) and potassium carbonate (1.5 g, 10.85 mmol) and nitrogen was bubbled through the suspension for 1 minute. The reaction was capped and heated to 80° C. for 2 hours. The mixture was cooled to ambient temperature and saturated ammonium chloride was added and the it was extracted with ethyl acetate. The combined organic extracts were washed with brine, dried over sodium sulfate, filtered and concentrated. The residue was purified by silica gel column chromatography (80 gram column) using a gradient of 100% hexanes to 50% ethyl acetate in hexanes (compound elutes at 30% EtOAc) to give a pale-yellow oil tert-butyl N-[4-[2-(benzyloxymethyl)-6-methyl-phenyl]-6-chloro-pyrimidin-2-yl]-N-tert-butoxycarbonyl-carbamate (1.73 g, 78%). ESI-MS m/z calc. 539.2187, found 540.2 (M+1)⁺; Retention time: 1.87 minutes; LC method Q. ¹H NMR (499 MHz, Chloroform-d) δ 7.39-7.35 (m, 2H), 7.35-7.30 (m, 3H), 7.29-7.23 (m, 4H), 4.40 (s, 2H), 4.30 (s, 2H), 2.13 (s, 3H), 1.44 (s, 18H).

### Step 5: 4-[2-(Benzyloxymethyl)-6-methyl-phenyl]-6-chloro-pyrimidin-2-amine

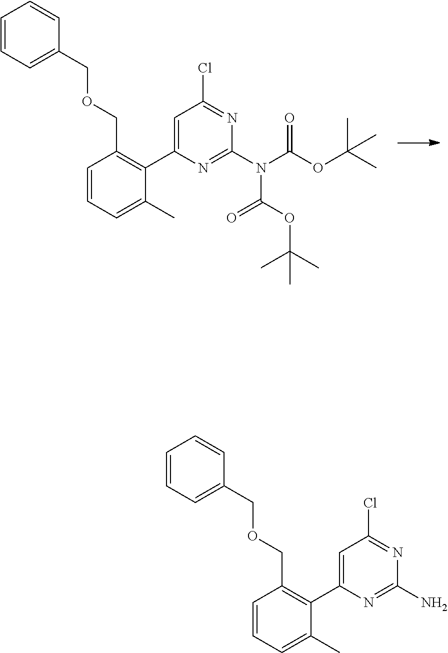 [SMILES: Cc1cccc(COCc2ccccc2)c1-c1cc(Cl)nc(N(C(=O)OC(C)(C)C)C(=O)OC(C)(C)C)n1 ; Cc1cccc(COCc2ccccc2)c1-c1cc(Cl)nc(N)n1]

tert-Butyl N-[4-[2-(benzyloxymethyl)-6-methyl-phenyl]-6-chloro-pyrimidin-2-yl]-N-tert-butoxycarbonyl-carbamate (1.7 g, 3.148 mmol) was dissolved in DCM (35 mL) and to the mixture was added HCl (4M in dioxane) (21 mL of 4 M, 84.00 mmol) and the reaction was stirred at room temperature. After 6 h, the mixture was evaporated to dryness, then diluted with ether (50 mL×2) and then hexanes: dichloromethane (1:1 mixture, 50 mL) and concentrated. The material was then placed on the high vacuum pump for 16 h to afford a pale-yellow gum as product 4-[2-(benzyloxymethyl)-6-methyl-phenyl]-6-chloro-pyrimidin-2-amine (1.07 g, 100%). ESI-MS m/z calc. 339.11383, found 340.2 (M+1)⁺; Retention time: 1.67 minutes; LC method A. H NMR (499 MHz, DMSO-d₆) δ 7.35-7.30 (m, 4H), 7.29-7.24 (m, 2H), 7.24-7.15 (m, 4H), 6.65 (s, 1H), 4.37 (s, 2H), 4.33 (s, 2H), 2.10 (s, 3H).

### Step 6: Methyl 3-[[4-[2-(benzyloxymethyl)-6-methyl-phenyl]-6-chloro-pyrimidin-2-yl]sulfamoyl]benzoate

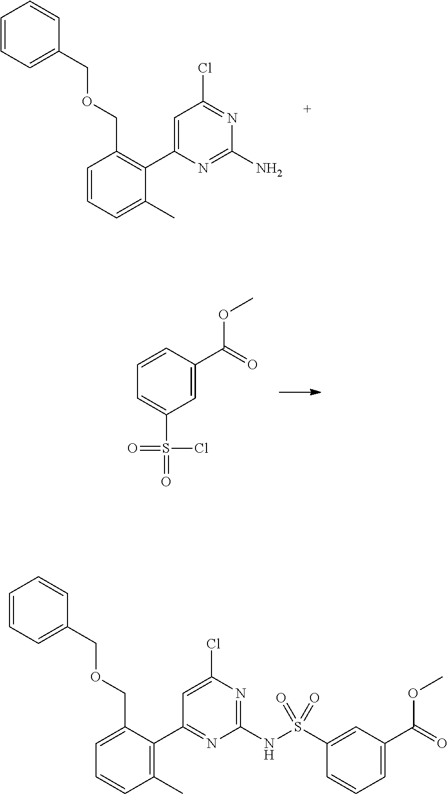 [SMILES: Cc1cccc(COCc2ccccc2)c1-c1cc(Cl)nc(N)n1 ; COC(=O)c1cccc(S(=O)(=O)Cl)c1 ; COC(=O)c1cccc(S(=O)(=O)Nc2nc(Cl)cc(-c3c(C)cccc3COCc3ccccc3)n2)c1]

4-[2-(benzyloxymethyl)-6-methyl-phenyl]-6-chloro-pyrimidin-2-amine (2.74 g, 8.063 mmol) was dissolved in THF (50 mL) and cooled in an ice bath to 0° C. methyl 3-chlorosulfonylbenzoate (2.85 g, 12.15 mmol) was added in one portion. lithium tert-amoxide (7.25 mL of 40% w/w, 22.50 mmol) was added dropwise and the reaction was allowed to slowly warm to room temperature. The reaction was stirred for 6 h. The mixture was pumped on high vacuum overnight, then re-subjected to dilution in THE (25 mL). After cooling in an ice bath to methyl 3-chlorosulfonylbenzoate (1.0 g) was added followed by the addition of lithium tert-amoxide (3 mL of 40% w/w) added dropwise and the reaction was allowed to warm at room temperature for 4 hours. The mixture was acidified the addition of 1 HCl. The reaction mixture was extracted with ethyl acetate. The organics were washed with brine, dried over sodium sulfate and evaporated. The crude material was purified utilizing silica gel column chromatography (120 gram column) using a gradient of 100% hexanes to 80% ethyl acetate in hexanes (compound elutes at 50% EtOAc) to give a pale-yellow oil which solidified upon high vacuum to produce methyl 3-[[4-[2-(benzyloxymethyl)-6-methyl-phenyl]-6-chloro-pyrimidin-2-yl]sulfamoyl]benzoate (2.06 g, 47%). ESI-MS m/z calc. 537.11255, found 538.2 (M+1)⁺; Retention time: 1.97 minutes; LC method A.

### Step 7: 3-[[4-[2-(Benzyloxymethyl)-6-methyl-phenyl]-6-chloro-pyrimidin-2-yl]sulfamoyl]benzoic Acid

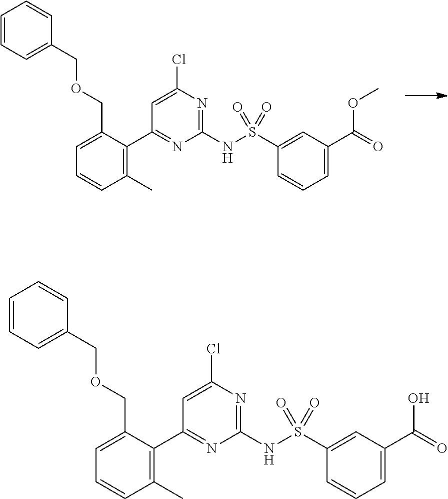 [SMILES: COC(=O)c1cccc(S(=O)(=O)Nc2nc(Cl)cc(-c3c(C)cccc3COCc3ccccc3)n2)c1 ; Cc1cccc(COCc2ccccc2)c1-c1cc(Cl)nc(NS(=O)(=O)c2cccc(C(=O)O)c2)n1]

Methyl 3-[[4-[2-(benzyloxymethyl)-6-methyl-phenyl]-6-chloro-pyrimidin-2-yl]sulfamoyl]benzoate (2.06 g, 3.829 mmol) and NaOH (30 mL of 1 M, 30.00 mmol) were combined in THE (25 mL) and stirred at room temperature for 2 h. The reaction was made acidic by the addition of 1M HCl and extracted with ethyl acetate. The organics were washed with brine, dried over sodium sulfate and evaporated. The material was placed on high vac overnight to give 3-[[4-[2-(benzyloxymethyl)-6-methyl-phenyl]-6-chloro-pyrimidin-2-yl]sulfamoyl]benzoic acid (1.95 g, 97%). ESI-MS m/z calc. 523.09686, found 524.1 (M+1)⁺; Retention time: 1.72 minutes; LC method A.

### Step 8: 3-[[4-[(2R)-2-Amino-4-methyl-pentoxy]-6-[2-(benzyloxymethyl)-6-methyl-phenyl]pyrimidin-2-yl]sulfamoyl]benzoic Acid

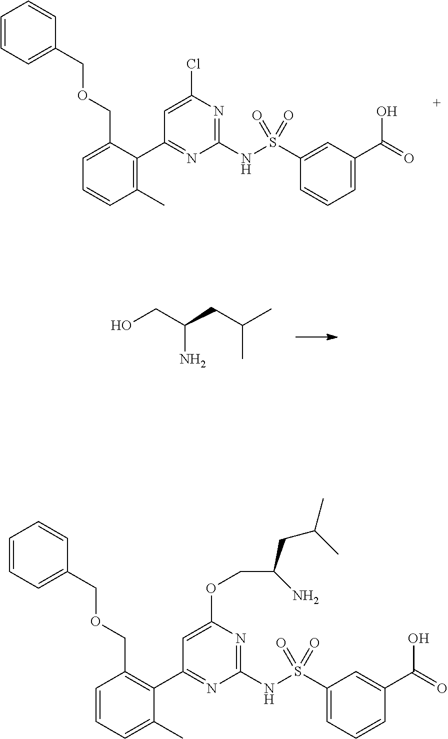

In a 500 mL flask, 3-[[4-[2-(benzyloxymethyl)-6-methyl-phenyl]-6-chloro-pyrimidin-2-yl]sulfamoyl]benzoic acid (2.0 g, 3.817 mmol), (2R)-2-amino-4-methyl-pentan-1-ol (460 mg, 3.925 mmol) and THE (40 mL) were mixed and cooled in an ice bath at 0° C., to which KOtBu (2.15 g, 19.16 mmol) was added. This mixture was stirred 2 h. The reaction was acidified by the addition of HCl (4M in dioxane) (7 mL of 4 M, 28.00 mmol), stirred for 15 minutes and then concentrated in vacuo. The material was rdissolved in DCM/ether and triturated, filtered and dried under high vacuum to afford off-white solid 3-[[4-[(2R)-2-amino-4-methyl-pentoxy]-6-[2-(benzyloxymethyl)-6-methyl-phenyl]pyrimidin-2-yl]sulfamoyl]benzoic acid (hydrochloride salt) (2.4 g, 98%). ESI-MS m/z calc. 604.23553, found 605.2 (M+1)⁺; Retention time: 1.29 minutes; LC method A.

### Step 9: (11R)-6-[2-(Benzyloxymethyl)-6-methyl-phenyl]-11-isobutyl-2,2-dioxo-9-oxa-2)⁶-thia-3,5,12,19-tetrazatricyclo[12.3.1.14,8]nonadeca-1(18),4(19),5,7,14,16-hexaen-13-one (Compound 106)

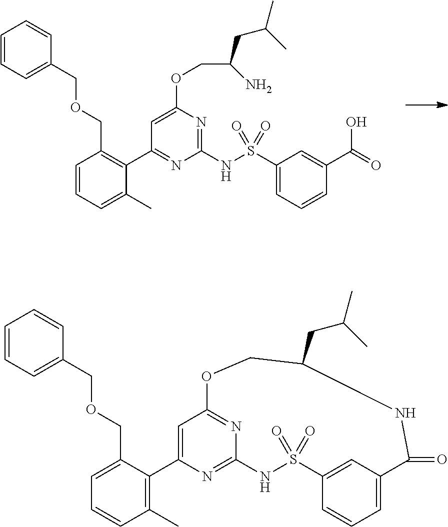 [SMILES: Cc1cccc(COCc2ccccc2)c1-c1cc(OC[C@H](N)CC(C)C)nc(NS(=O)(=O)c2cccc(C(=O)O)c2)n1 ; Cc1cccc(COCc2ccccc2)c1-c1cc2nc(n1)NS(=O)(=O)c1cccc(c1)C(=O)N[C@H](CC(C)C)CO2]

In a 100-mL flask, 3-[[4-[(2R)-2-amino-4-methyl-pentoxy]-6-[2-(benzyloxymethyl)-6-methyl-phenyl]pyrimidin-2-yl]sulfamoyl]benzoic acid (hydrochloride salt) (375 mg, 0.5849 mmol) was dissolved in DMF (12 mL), to which HATU (400 mg, 1.052 mmol) and then DIPEA (900 μL, 5.167 mmol) were added. After stirring at room temperature for 30 min, The mixture was diluted with water and ethyl acetate, and the aqueous layer was extracted (3×75 mL). The combined organic extract was washed with water (50 mL) and saturated brine solution (50 mL), then dried over sodium sulfate, filtered, and evaporated in vacuo. This crude product was purified by silica gel chromatography using a 40 g column eluting with 100% dichloromethane to 15% methanol in dichloromethane to afford a light orange solid then was further purified by reverse-phase preparative chromatography utilizing a C₁₈ column and a 1-99% gradient over 15 min of acetonitrile in water (+5 mM HCl) to give as a white solid (11R)-6-[2-(benzyloxymethyl)-6-methyl-phenyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (102.34 mg, 30%). ESI-MS m/z calc. 586.225, found 587.2 (M+1)⁺; Retention time: 1.68 minutes; LC method A. H NMR (499 MHz, DMSO-d₆) δ 13.15 (s, 1H), 8.52 (s, 1H), 8.03-7.80 (m, 2H), 7.68 (s, 2H), 7.39-7.02 (m, 7H), 6.48 (d, J 43.4 Hz, 1H), 5.16 (d, J 10.4 Hz, 1H), 4.32 (d, J 11.5 Hz, 2H), 4.17 (d, J 50.8 Hz, 2H), 3.86 (t, J 11.0 Hz, 1H), 2.08 (s, 5H), 1.54-1.37 (m, 2H), 1.18 (t, J 12.1 Hz, 1H), 0.72 (s, 3H), 0.21 (s, 3H).

### Example 100: Preparation of Compound 107

### Step 1: (2-Bromo-3-methyl-phenyl)-cyclopropyl-methanol

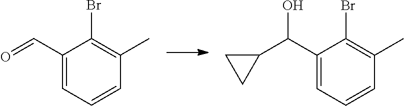

To a solution of 2-bromo-3-methyl-benzaldehyde (1 g, 5.024 mmol) in dry THE (10 mL) at 0° C. under nitrogen atmosphere was added bromo(cyclopropyl)magnesium (5.6 mL of 1 M, 5.600 mmol) and the reaction mixture was stirred for 2 h slowly warming up to RT. The reaction was quenched with saturated ammonium chloride solution and extracted with ethyl acetate. The combined organics were washed with a brine, dried over sodium sulfate and evaporated. The resultant brown residue was purified by silica gel column chromatography using a shallow gradient 100% hexanes to 100% EtOAc to afford (2-bromo-3-methyl-phenyl)-cyclopropyl-methanol (857 mg, 71%) as a colorless oil. ESI-MS m/z calc. 240.01498, found 242.13 (M+1)⁺; Retention time: 0.62 minutes; LC method D.

### Step 2: 2-Bromo-1-(cyclopropylmethyl)-3-methyl-benzene

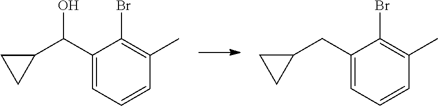

To a solution of (2-bromo-3-methyl-phenyl)-cyclopropyl-methanol (857 mg, 3.554 mmol) in DCM (5 mL) was added (BF3.diethylether) diethyloxonio(trifluoro)boranuide (700 μL, 5.672 mmol) and triethylsilane (700 μL, 4.383 mmol) at 0° C. and the reaction mixture was stirred for 5 h by allowing the temperature to warm up to rt. The reaction mixture was quenched with saturated ammonium chloride solution and extracted with DCM, combined organic layers washed with brine, dried over anhydrous magnesium sulphate, filtered and concentrated. The resultant brown residue was purified by silica gel column chromatography using a gradient 100% hexanes to 20% EtOAc to afford 2-bromo-1-(cyclopropylmethyl)-3-methyl-benzene (480 mg, 60%). as an oil. ESI-MS m/z calc. 224.02007, found 227.08 (M+1)⁺; Retention time: 0.86 minutes; LC method D. ¹H NMR (500 MHz, DMSO-d₆) δ 7.23-7.10 (m, 3H), 2.60 (d, J 7.2 Hz, 2H), 2.35 (s, 3H), 1.03 (ddt, J 10.1, 7.5, 3.7 Hz, 1H), 0.49-0.41 (m, 2H), 0.20 (dt, J 5.9, 4.2 Hz, 2H).

### Step 3: 2-[2-(Cyclopropylmethyl)-6-methyl-phenyl]-4,4,5,5-tetramethyl-1,3,2-dioxaborolane

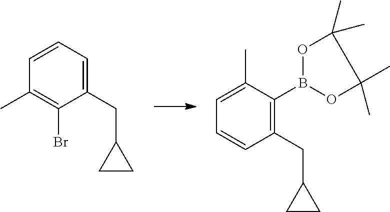 [SMILES: Cc1cccc(CC2CC2)c1Br ; Cc1cccc(CC2CC2)c1B1OC(C)(C)C(C)(C)O1]

To a solution of 2-bromo-1-(cyclopropylmethyl)-3-methyl-benzene (480 mg, 2.132 mmol) in dioxane (5 mL) was added 4,4,5,5-tetramethyl-2-(4,4,5,5-tetramethyl-1,3,2-dioxaborolan-2-yl)-1,3,2-dioxaborolane (658 mg, 2.591 mmol) and KOAc (456 mg, 4.646 mmol) and the mixture was degassed with nitrogen for 5 minutes. Then Pd(dppf)Cl₂ (367 mg, 0.4494 mmol) was added and the reaction was heated at 80° C. for under nitrogen balloon for 16 hours. After the reaction was cooled to room temperature, it was diluted with ethylate and filtered through a pad of Celite and the filtrate was concentrated. The resultant brown residue was purified by silica gel column chromatography using a shallow gradient 100% hexanes to 20% EtOAc to afford 2-[2-(cyclopropylmethyl)-6-methyl-phenyl]-4,4,5,5-tetramethyl-1,3,2-dioxaborolane (285 mg, 49%) as viscous oil. ESI-MS m/z calc. 272.19476, found 273.28 (M+1)⁺; Retention time: 1.34 minutes; LC method Q.

### Step 4: (11R)-6-[2-(Cyclopropylmethyl)-6-methyl-phenyl]-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

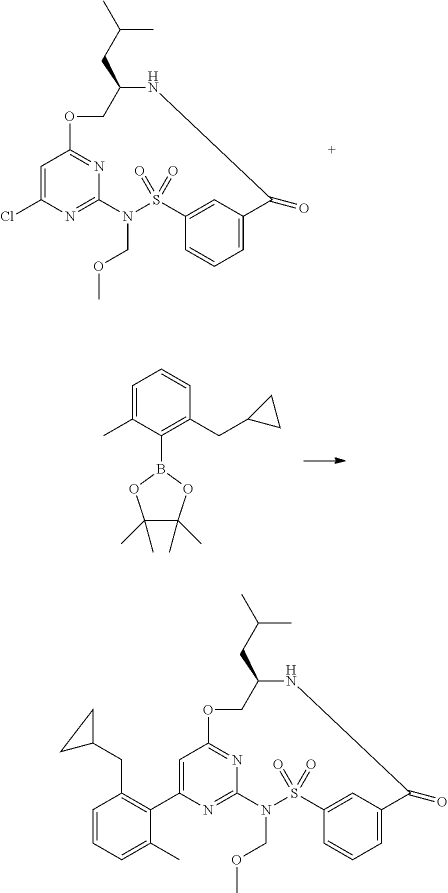 [SMILES: COCN1c2nc(Cl)cc(n2)OC[C@@H](CC(C)C)NC(=O)c2cccc(c2)S1(=O)=O ; Cc1cccc(CC2CC2)c1B1OC(C)(C)C(C)(C)O1 ; COCN1c2nc(cc(-c3c(C)cccc3CC3CC3)n2)OC[C@@H](CC(C)C)NC(=O)c2cccc(c2)S1(=O)=O]

To a solution of (11R)-6-chloro-11-isobutyl-3-(methoxymethyl)-2,2-dioxo-9-oxa-2λ⁶-thia-3,5,12,19-tetrazatricyclo[12.3.1.14,8]nonadeca-1(18),4,6,8(19),14,16-hexaen-13-one (50 mg, 0.1099 mmol) in DMA (2 mL) in a microwave vial was added 2-[2-(cyclopropylmethyl)-6-methyl-phenyl]-4,4,5,5-tetramethyl-1,3,2-dioxaborolane (38 mg, 0.1396 mmol) and the solution was purged with a continuous stream of nitrogen for 5 min. Pd(dppf)Cl₂ (18 mg, 0.02204 mmol) and potassium carbonate (280 μL of 2 M, 0.5600 mmol) was added and the orange color solution was purged with a continuous stream of nitrogen for 5 min. The vial was capped and heated at 100° C. for 1 h. The reaction mixture was cooled to rt and diluted with ethylacetate and filtered through Celite. The filtrate was concentrated and dissolved in 2 mL of DMSO and purified by a reverse phase HPLC-MS method using a dual gradient run from 50-99% mobile phase B over 15.0 minutes. Mobile phase A=H₂O (5 mM HCl). Mobile phase B=CH₃CN to afford (11R)-6-[2-(cyclopropylmethyl)-6-methyl-phenyl]-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (12 mg, 19%) as off white solid. ESI-MS m/z calc. 564.24066, found 565.5 (M+1)⁺; Retention time: 0.41 minutes; LC method Q using a 1 min gradient.

### Step 5: (11R)-6-[2-(Cyclopropylmethyl)-6-methyl-phenyl]-11-isobutyl-2,2-dioxo-9-oxa-2)⁶-thia-3,5,12,19-tetrazatricyclo[12.3.1.14,8]nonadeca-1(18),4(19),5,7,14,16-hexaen-13-one (Compound 107)

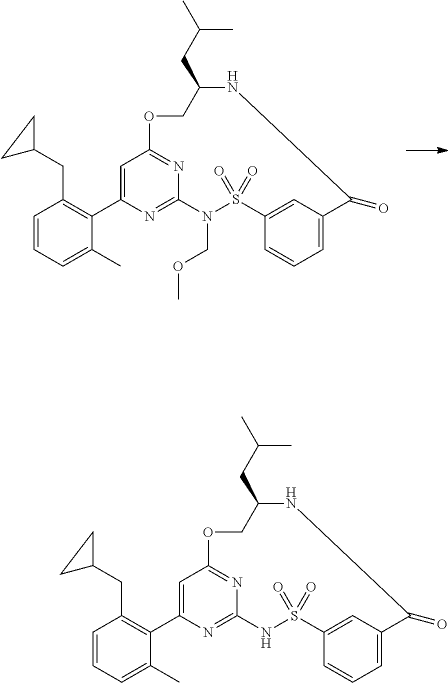 [SMILES: COCN1c2nc(cc(-c3c(C)cccc3CC3CC3)n2)OC[C@@H](CC(C)C)NC(=O)c2cccc(c2)S1(=O)=O ; Cc1cccc(CC2CC2)c1-c1cc2nc(n1)NS(=O)(=O)c1cccc(c1)C(=O)N[C@H](CC(C)C)CO2]

A solution of (11R)-6-[2-(cyclopropylmethyl)-6-methyl-phenyl]-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (10 mg, 0.01771 mmol) in (1: 4 TFA-DCM premixed solution) DCM (600 μL) and TFA (150 μL, 1.947 mmol) was stirred at rt for 16 h. The solvent was removed and the residue was purified by reverse phase HPLC-MS using a dual gradient of 30-99% mobile phase B over 15.0 minutes. Mobile phase A=H₂O (5 mM HCl). Mobile phase B=CH₃CN to afford (11R)-6-[2-(cyclopropylmethyl)-6-methyl-phenyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (5.4 mg, 56%) as white solid. ESI-MS m/z calc. 520.2144, found 521.15 (M+1)⁺; Retention time: 1.2 minutes; LC method Q. ¹H NMR (500 MHz, DMSO-d₆) δ 13.10 (s, 1H), 8.50 (s, 1H), 7.91 (d, J 13.6 Hz, 2H), 7.68 (s, 1H), 7.30 (d, J 27.3 Hz, 2H), 7.15 (d, J 18.4 Hz, 1H), 6.66-6.17 (m, −1H), 5.16 (d, J=10.6 Hz, 1H), 3.89 (t, J=11.0 Hz, 1H), 3.35-3.29 (m, 2H), 2.33 (s, 1H), 2.13 (s, 3H), 1.92 (s, 1H), 1.50 (d, J 10.9 Hz, 2H), 1.19 (d, J 11.9 Hz, 1H), 0.77 (d, J 6.4 Hz, 3H), 0.44 (s, 1H), 0.26 (s, 5H), −0.09 (d, J 30.7 Hz, 2H).

### Example 101: Preparation of Compound 108

### Step 1: (11R)-11-Isobutyl-6-(3-methyl-2-pyridyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 108)

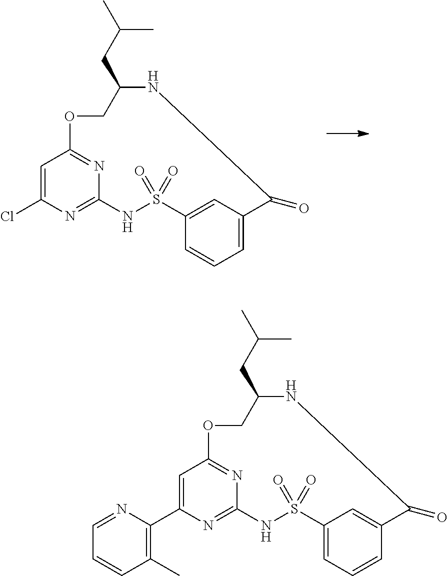 [SMILES: CC(C)C[C@@H]1COc2cc(Cl)nc(n2)NS(=O)(=O)c2cccc(c2)C(=O)N1 ; Cc1cccnc1-c1cc2nc(n1)NS(=O)(=O)c1cccc(c1)C(=O)N[C@H](CC(C)C)CO2]

(11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (25 mg, 0.06085 mmol), tributyl-(3-methyl-2-pyridyl)stannane (70 mg, 0.1832 mmol), potassium carbonate (33 mg, 0.2388 mmol), CuI (2.5 mg, 0.01313 mmol), and Pd(dppf)Cl₂ (5 mg, 0.006123 mmol) were combined in a nitrogen purged screwcap vial in DMF (0.5 mL) and stirred for 3 hours at 80° C. The reaction mixture was then diluted with 0.5 M HCl and extracted 3× with ethyl acetate. UPLC showed substantial product remained in the aqueous layer, so an equal volume of brine was added and the aqueous was extracted an additional 4× ethyl acetate. The combined organics were washed with brine, dried over sodium sulfate and concentrated. The resulting crude was dissolved in 1:1 DMSO/methanol, filtered, and purified by reverse phase HPLC (1-70% ACN in water, HCl modifier) to give (11R)-11-isobutyl-6-(3-methyl-2-pyridyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (12 mg, 38%). ESI-MS m/z calc. 467.16272, found 468.2 (M+1)⁺; Retention time: 1.15 minutes; LC method A.

### Example 102: Preparation of (11R)-6-(2-methylphenoxy)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione

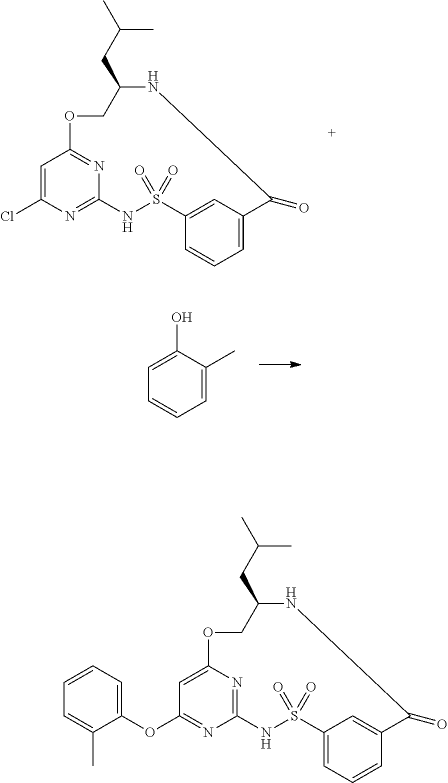

A solution of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (30 mg, 0.07301 mmol) in DMF (0.4 mL) was added to o-cresol (approximately 15.79 mg, 28.57 μL, 0.1460 mmol). Sodium hydride (approximately 7.007 mg, 0.2920 mmol) was added. After stirring at room temperature for 15 minutes, the reaction mixture was allowed to stir at 80° C. for 1 hour, then 110° C. for 1 hour and 120° C. for 5 hours. After filtration, the product was isolated by UV-triggered reverse-phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-6-(2-methylphenoxy)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (4.1 mg, 12%) was obtained. ESI-MS m/z calc. 482.16238, found 483.3 (M+1)⁺; Retention time: 1.75 minutes; LC method A.

### Example 103: Preparation of Compound 109

### Step 1: (11R)-6-[3-methyl-5-(trifluoromethyl)-1H-pyrazol-1-yl]-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 109)

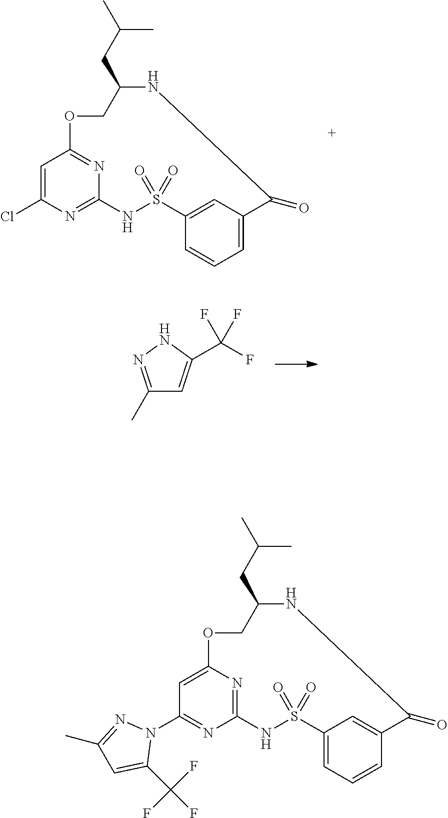

A solution of (11R)-6-chloro-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (30 mg, 0.07301 mmol) in DMF (0.4 mL) was added to 3-methyl-5-(trifluoromethyl)-1H-pyrazole (approximately 21.91 mg, 0.1460 mmol). Sodium hydride (approximately 7.007 mg, 0.2920 mmol) was added. After stirring at room temperature for 15 minutes, the reaction mixture was allowed to stir at 80° C. for 1 hour, then 110° C. for 1 hour and 120° C. for 5 hours. After filtration, the product was isolated by UV-triggered reverse-phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-6-[3-Methyl-5-(trifluoromethyl)-1H-pyrazol-1-yl]-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (4.8 mg, 13%) was obtained. ESI-MS m/z calc. 524.1454, found 525.3 (M+1)⁺; Retention time: 1.89 minutes; LC method A.

### Example 104: Preparation of Compound 110 and Compound 111

### Step 1: 3-(1-Adamantyl)-2-amino-propan-1-ol

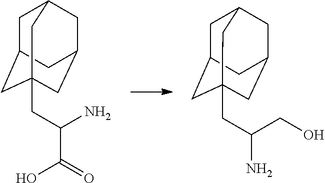 [SMILES: NC(CC12CC3CC(CC(C3)C1)C2)C(=O)O ; NC(CO)CC12CC3CC(CC(C3)C1)C2]

3-(1-Adamantyl)-2-amino-propanoic acid (hydrochloride salt) (240 mg, 0.9239 mmol) was dissolved in anhydrous THE (4.619 mL) in a nitrogen-purged flask and cooled to 0° C. in an ice bath. LAH (3 mL of 1 M, 3.000 mmol) (1M in THF) was added dropwise. The reaction was allowed to warm to room temperature as the ice melted and stirred for 16 hours at room temperature. The reaction mixture was then cooled again to 0° C. and quenched by the dropwise addition of 0.15 mL water. After the bubbling ceased, 0.15 mL 15% NaOH was added followed by 0.5 mL water. The reaction mixture was allowed to warm to room temperature and was stirred for 30 minutes. Magnesium sulfate was added, and the reaction mixture was filtered through Celite, eluting with diethyl ether. The Celite filter cake was washed with an excess of methanol, then concentrated to give a large quantity of a white solid. This material was stirred in THF/3M NaOH(aq) for 8 hours, then extracted 2×diethyl ether, 2×ethyl acetate. The combined organics were washed with brine, dried over sodium sulfate, and concentrated, to give additional amino alcohol, which was combined with the material recovered from the initial filtrate. The filtrate was concentrated, and the 3-(1-adamantyl)-2-amino-propan-1-ol (102 mg, 53%) was used in the next step without purification. ESI-MS m/z calc. 209.17796, found 210.1 (M+1)⁺; Retention time: 0.4 minutes; LC method D.

### Step 2: 3-[[4-[3-(1-adamantyl)-2-amino-propoxy]-6-(2,6-dimethylphenyl)pyrimidin-2-yl]sulfamoyl]benzoic Acid

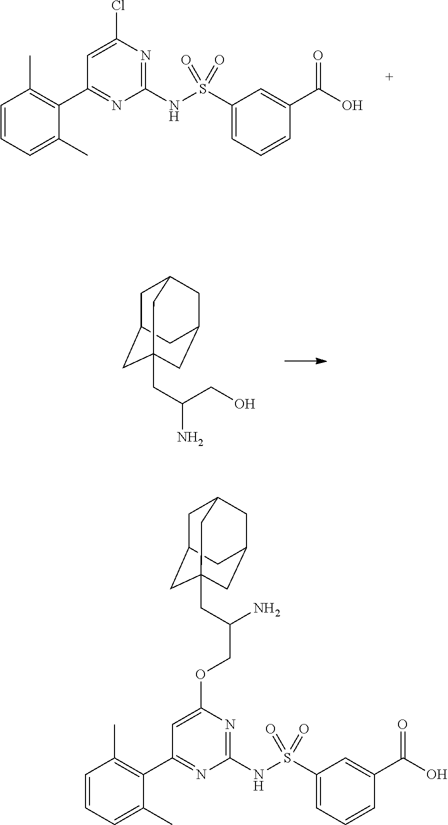 [SMILES: Cc1cccc(C)c1-c1cc(Cl)nc(NS(=O)(=O)c2cccc(C(=O)O)c2)n1 ; NC(CO)CC12CC3CC(CC(C3)C1)C2 ; Cc1cccc(C)c1-c1cc(OCC(N)CC23CC4CC(CC(C4)C2)C3)nc(NS(=O)(=O)c2cccc(C(=O)O)c2)n1]

3-[[4-Chloro-6-(2,6-dimethylphenyl)pyrimidin-2-yl]sulfamoyl]benzoic Acid (approximately 144.0 mg, 0.3445 mmol) and 3-(1-adamantyl)-2-amino-propan-1-ol (101 mg, 0.4825 mmol) were combined in anhydrous THE (765.6 μL). Sodium tert-butoxide (approximately 132.4 mg, 1.378 mmol) was added, and the reaction mixture was stirred at 60° C. for 20 minutes. After cooling to room temperature Boc anhydride (approximately 150.4 mg, 158.3 μL, 0.6890 mmol) was added. The reaction was stirred for 2 hours at room temperature with only partial boc protection observed by UPLC. An additional portion of Boc anhydride (approximately 150.4 mg, 158.3 μL, 0.6890 mmol) was added. After a further two hours the reaction mixture was diluted with ethyl acetate and washed with 0.5 M HCl. The layers were separated and the aqueous was extracted an additional 3× with ethyl acetate. The combined organics were washed with brine, dried over sodium sulfate, and concentrated. The crude material was purified by chromatography on silica gel, eluting with 0-10% methanol in DCM. Fractions containing product were combined and concentrated. The product was dissolved in dichloromethane, and HCl (approximately 861.2 μL of 4 M, 3.445 mmol) in dioxane was added. The reaction was stirred at room temperature for 45 minutes then concentrated. Dichloromethane and hexanes were added and the reaction mixture was concentrated again to give a white solid 3-[[4-[3-(1-adamantyl)-2-amino-propoxy]-6-(2,6-dimethylphenyl)pyrimidin-2-yl]sulfamoyl]benzoic acid (hydrochloride salt) (55 mg, 25%) which was used in the next step without further purification. ESI-MS m/z calc. 590.2563, found 591.3 (M+1)⁺; Retention time: 0.52 minutes; LC method D.

### Step 3: 11-(1-adamantylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, enantiomer 1 (Compound 110), and 11-(1-adamantylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, enantiomer 2 (Compound 111)

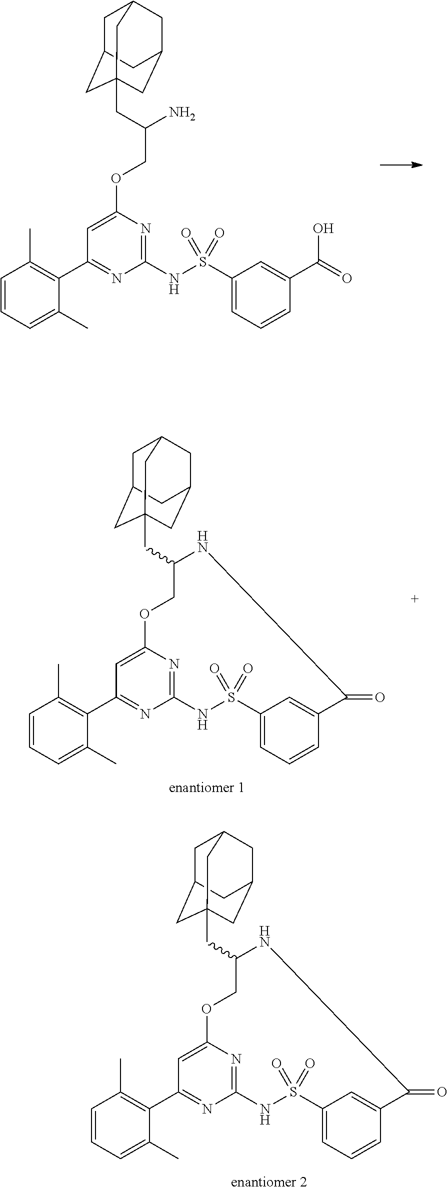 [SMILES: Cc1cccc(C)c1-c1cc(OCC(N)CC23CC4CC(CC(C4)C2)C3)nc(NS(=O)(=O)c2cccc(C(=O)O)c2)n1 ; Cc1cccc(C)c1-c1cc2nc(n1)NS(=O)(=O)c1cccc(c1)C(=O)NC(CC13CC4CC(CC(C4)C1)C3)CO2 ; Cc1cccc(C)c1-c1cc2nc(n1)NS(=O)(=O)c1cccc(c1)C(=O)NC(CC13CC4CC(CC(C4)C1)C3)CO2]

3-[[4-[3-(1-Adamantyl)-2-amino-propoxy]-6-(2,6-dimethylphenyl)pyrimidin-2-yl]sulfamoyl]benzoic acid (hydrochloride salt) (65 mg, 0.1036 mmol) was dissolved in DMF (1 mL) and added several dros at a time over the course of one hour to a stirring solution of HATU (80 mg, 0.2104 mmol) and DIPEA (110 μL, 0.6315 mmol) in DMF (10 mL). When the addition was complete, the reaction mixture was poured into a separatory funnel containing 50 mL 1M HCl and 50 mL ethyl acetate. The layers were separated and the aqueous was extracted with an additional 2×25 mL ethyl acetate. The combined organics were washed with water followed by brine, dried over sodium sulfate, filtered, and concentrated. The resulting crude material was purified by chromatography on silica gel (0-10% methanol in DCM) to give a slightly yellow solid (very poor solubility in most solvents), 11-(1-adamantylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (41 mg, 69%). This racemic material was subjected to an analytical screen of chiral SFC methods (see reaction attachment) and the chiral LUX-4 column was selected as the best available separation. This material was dissolved in 2.5 mL DMSO, and chiral separation was conducted using the walkup SFC instrument with a 50-80% methanol gradient and 0.15 to 0.3 mL injections. This gave as a white solid enantiomer 1, peak 1, 11-(1-adamantylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (11.4 mg, 19%) ESI-MS m/z calc. 572.2457, found 573.3 (M+1)⁺; Retention time: 1.82 minutes (LC method A); and enantiomer 2, peak 2 11-(1-adamantylmethyl)-6-(2,6-dimethylphenyl)-2,2-dioxo-9-oxa-2⁶-thia-3,5,12,19-tetrazatricyclo[12.3.1.14,8]nonadeca-1(18),4(19),5,7,14,16-hexaen-13-one (8.7 mg, 15%) ESI-MS m/z calc. 572.2457, found 573.3 (M+1)⁺; Retention time: 1.81 minutes (LC method A).

### Example 105: Preparation of Compound 112

### Step 1: 3-({4-[(2R)-2-{[(tert-Butoxy)carbonyl]amino}-3-methylbutoxy]-6-(2,6-dimethylphenyl)pyrimidin-2-yl}sulfamoyl)benzoic acid

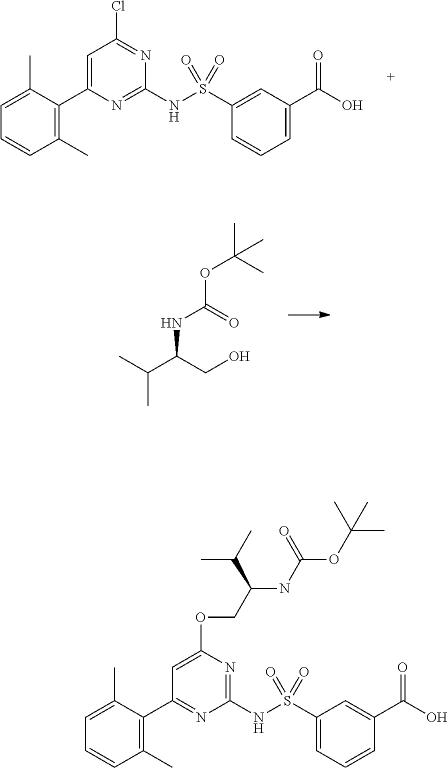 [SMILES: Cc1cccc(C)c1-c1cc(Cl)nc(NS(=O)(=O)c2cccc(C(=O)O)c2)n1 ; CC(C)[C@H](CO)NC(=O)OC(C)(C)C ; Cc1cccc(C)c1-c1cc(OC[C@H](NC(=O)OC(C)(C)C)C(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (70 mg, 0.1675 mmol) in THE (653.3 μL) was added to tert-butyl N-[(1R)-1-(hydroxymethyl)-2-methyl-propyl]carbamate (approximately 51.08 mg, 0.2513 mmol). Solid sodium tert-butoxide (approximately 80.49 mg, 0.8375 mmol) was added last. The reaction mixture was allowed to stir overnight at room temperature. The reaction mixture was neutralized with the addition of aqueous HCl. The remaining suspension was diluted with DMSO (200 μL), filtered and purified by reverse phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. 3-({4-[(2R)-2-{[(tert-butoxy)carbonyl]amino}-3-methylbutoxy]-6-(2,6-dimethylphenyl)pyrimidin-2-yl}sulfamoyl)benzoic acid was obtained (97 mg). ESI-MS m/z calc. 584.23047, found 585.2 (M+1)⁺; Retention time: 1.73 minutes; LC method A.

### Step 2 (11R)-6-(2,6-Dimethylphenyl)-11-(propan-2-yl)-9-oxa-2λ⁶-thia-3,5,12,19-tetraazatricyclo[12.3.1.14,8]nonadeca-1(17),4,6,8(19),14(18),15-hexaene-2,2,13-trione (Compound 112)

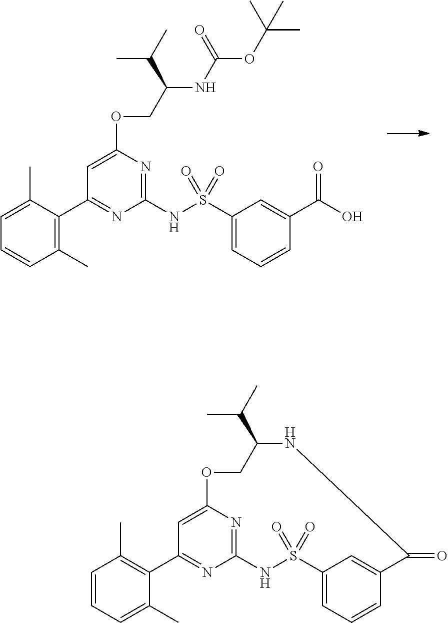 [SMILES: Cc1cccc(C)c1-c1cc(OC[C@H](NC(=O)OC(C)(C)C)C(C)C)nc(NS(=O)(=O)c2cccc(C(=O)O)c2)n1 ; Cc1cccc(C)c1-c1cc2nc(n1)NS(=O)(=O)c1cccc(c1)C(=O)N[C@H](C(C)C)CO2]

3-({4-[(2R)-2-{[(tert-Butoxy)carbonyl]amino}-3-methylbutoxy]-6-(2,6-dimethylphenyl)pyrimidin-2-yl}sulfamoyl)benzoic acid (97.93 mg, 0.1675 mmol) was dissolved in a 4 N solution of HCl (approximately 963.0 μL of 4 M, 3.852 mmol) in dioxane (1 mL). After stirring at room temperature for 15 minutes, volatiles were removed under reduced pressure. The remaining residue was dissolved in DMF (1.5 mL). HATU (approximately 70.08 mg, 0.1843 mmol) was added followed by triethylamine (50 μL, 0.3587 mmol). The final reaction mixture was allowed to stir at room temperature for 15 minutes. After filtration, the solution was purified by reverse phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. After concentration, (11R)-6-(2,6-dimethylphenyl)-11-(propan-2-yl)-9-oxa-2λ⁶-thia-3,5,12,19-tetraazatricyclo[12.3.1.14,8]nonadeca-1(17),4,6,8(19),14(18),15-hexaene-2,2,13-trione (10.6 mg, 14%) was obtained. ESI-MS m/z calc. 466.16748, found 467.3 (M+1)⁺; Retention time: 1.44 minutes; LC method A.

### Example 106: Preparation of Compound 113

### Step 1: tert-Butyl N-[(1R)-1-(cyclobutylmethyl)-2-hydroxy-ethyl]carbamate

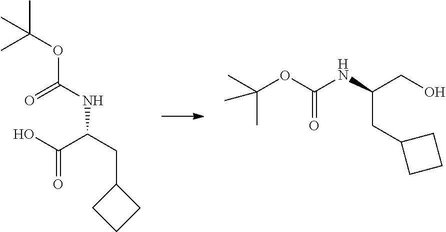 [SMILES: CC(C)(C)OC(=O)N[C@H](CC1CCC1)C(=O)O ; CC(C)(C)OC(=O)N[C@@H](CO)CC1CCC1]

To (2R)-2-(tert-butoxycarbonylamino)-3-cyclobutyl-propanoic acid (150 mg, 0.6165 mmol), cooled in an ice bath was added a solution of borane-THF (approximately 1.850 mL of 1 M, 1.850 mmol) (1M in THF). After 5 minutes the ice bath was removed, and the reaction mixture was stirred at room temperature for 2 hours. After this time the reaction mixture was poured into 1M aqueous citric acid, and the resulting solution was extracted 2× with ethyl acetate. The combined organics were washed with water, then brine, dried over sodium sulfate and concentrated, to give tert-butyl N-[(1R)-1-(cyclobutylmethyl)-2-hydroxy-ethyl]carbamate (140 mg, 99%), which were used in the next step without further purification. ESI-MS m/z calc. 229.1678, found 230.2 (M+1)⁺; Retention time: 0.56 minutes; LC method D.

### Step 2: 3-[[4-[(2R)-2-(tert-Butoxycarbonylamino)-3-cyclobutyl-propoxy]-6-(2,6-dimethylphenyl)pyrimidin-2-yl]sulfamoyl]benzoic Acid

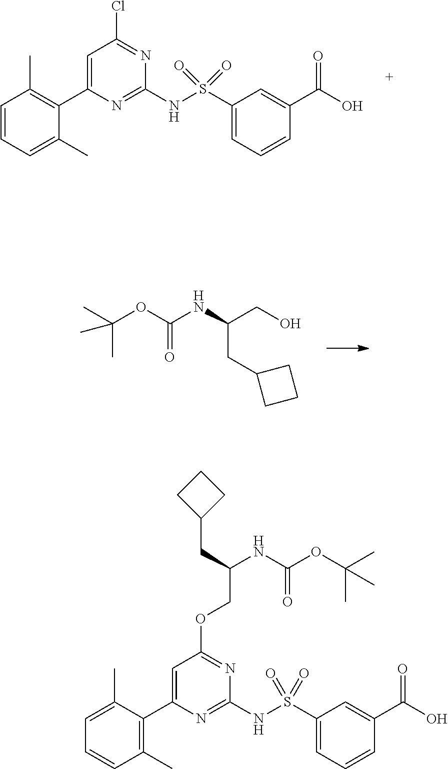 [SMILES: Cc1cccc(C)c1-c1cc(Cl)nc(NS(=O)(=O)c2cccc(C(=O)O)c2)n1 ; CC(C)(C)OC(=O)N[C@@H](CO)CC1CCC1 ; Cc1cccc(C)c1-c1cc(OC[C@@H](CC2CCC2)NC(=O)OC(C)(C)C)nc(NS(=O)(=O)c2cccc(C(=O)O)c2)n1]

3-[[4-Chloro-6-(2,6-dimethylphenyl)pyrimidin-2-yl]sulfamoyl]benzoic acid (75 mg, 0.1795 mmol), the tert-butyl N-[(1R)-1-(cyclobutylmethyl)-2-hydroxy-ethyl]carbamate (139 mg, 0.6062 mmol), and sodium tert-butoxide (approximately 138.0 mg, 1.436 mmol) were combined in THF (0.5 mL), and stirred at room temperature for 16 hours. The reaction mixture was then acidified with 0.25 mL acetic acid, diluted slightly with methanol, filtered, and purified by reverse phase HPLC (1-99% ACN in water, with HCl, 15 min run), then dried to give the corresponding 3-[[4-[(2R)-2-(tert-butoxycarbonylamino)-3-cyclobutyl-propoxy]-6-(2,6-dimethylphenyl)pyrimidin-2-yl]sulfamoyl]benzoic acid (41 mg, 37%) as a white powder. ESI-MS m/z calc. 610.2461, found 611.4 (M+1)⁺; Retention time: 0.73 minutes; LC method D.

### Step 3: (11R)-11-(cyclobutylmethyl)-6-(2,6-dimethylphenyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 113)

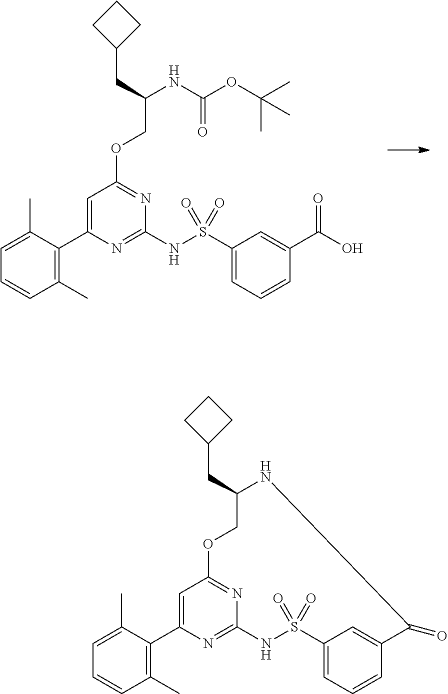 [SMILES: Cc1cccc(C)c1-c1cc(OC[C@@H](CC2CCC2)NC(=O)OC(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CC1CCC1)CO2]

3-[[4-[(2R)-2-(tert-Butoxycarbonylamino)-3-cyclobutyl-propoxy]-6-(2,6-dimethylphenyl)pyrimidin-2-yl]sulfamoyl]benzoic acid (40 mg, 0.06550 mmol) was dissolved in DCM (0.5 mL) and HCl (0.5 mL of 4 M, 2.000 mmol) in dioxane was added. After stirring for 30 minutes at room temperature, the reaction mixture was evaporated to give the Boc-deprotected amine, which was used in the next step without further purification. The amine product was combined with HATU (17 mg, 0.04471 mmol) in DMF (1 mL), and DIPEA (30 μL, 0.1722 mmol) was added. The reaction mixture was stirred at room temperature for 1 hour. After this time the reaction mixture was filtered, and purified by reverse phase HPLC (1-70% ACN) to give (11R)-11-(cyclobutylmethyl)-6-(2,6-dimethylphenyl)-9-oxa-2λ⁶-thia-3,5,12,19-tetraazatricyclo[12.3.1.14,8]nonadeca-1(17),4,6,8(19),14(18),15-hexaene-2,2,13-trione (6.9 mg, 21%). ESI-MS m/z calc. 492.18314, found 493.4 (M+1)⁺; Retention time: 1.59 minutes; LC method A.

### Example 107: Preparation of Compound 114

### Step 1: tert-Butyl N-[(1R)-1-(cyclopropylmethyl)-2-hydroxy-ethyl]carbamate

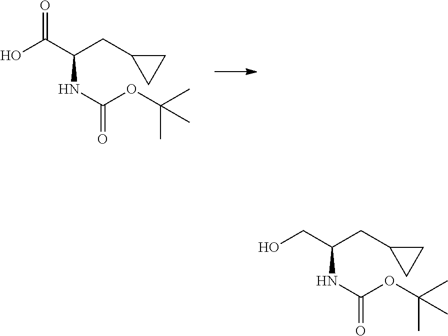 [SMILES: CC(C)(C)OC(=O)N[C@H](CC1CC1)C(=O)O ; CC(C)(C)OC(=O)N[C@@H](CO)CC1CC1]

A solution of (2R)-2-(tert-butoxycarbonylamino)-3-cyclopropyl-propanoic acid (0.22 g, 0.9596 mmol) and Borane-Tetrahydrofuran Complex (2.9 mL of 1 M, 2.900 mmol) in THE (5 mL) was stirred for three hours. The reaction was quenched with 1 M citric acid and extracted with ethyl acetate. The combined extracts were washed with water, dried over sodium sulfate, and evaporated under vacuum to give tert-butyl N-[(1R)-1-(cyclopropylmethyl)-2-hydroxy-ethyl]carbamate (89 mg, 43%). ESI-MS m/z calc. 215.15215, found 216.2 (M+1)⁺; Retention time: 0.47 minutes; LC method D.

### Step 2: 3-[[4-[(2R)-2-(tert-Butoxycarbonylamino)-3-cyclopropyl-propoxy]-6-(2,6-dimethylphenyl)pyrimidin-2-yl]sulfamoyl]benzoic Acid

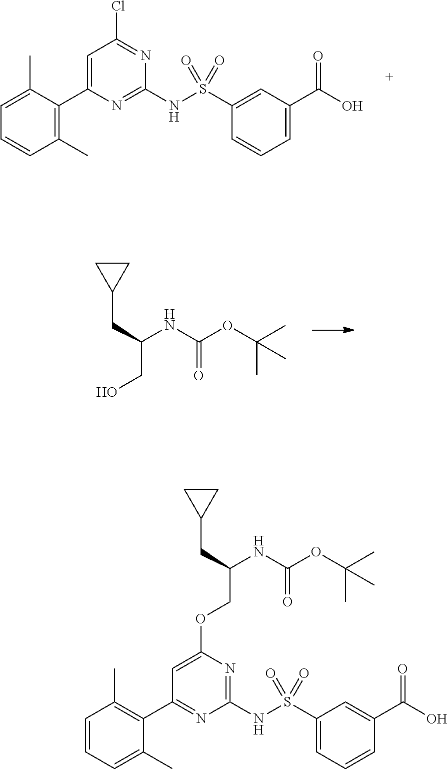 [SMILES: Cc1cccc(C)c1-c1cc(Cl)nc(NS(=O)(=O)c2cccc(C(=O)O)c2)n1 ; CC(C)(C)OC(=O)N[C@@H](CO)CC1CC1 ; Cc1cccc(C)c1-c1cc(OC[C@@H](CC2CC2)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (approximately 172.7 mg, 0.4134 mmol), tert-Butyl N-[(1R)-1-(cyclopropylmethyl)-2-hydroxy-ethyl]carbamate (89 mg, 0.4134 mmol), and sodium t-butoxide (approximately 159.0 mg, 1.654 mmol) in THE (2.067 mL) was stirred for 22 hours. The reaction was quenched with 1 M citric acid, diluted with water, and extracted with ethyl acetate. The combined extracts were washed with brine, dried over sodium sulfate, and evaporated. The residue was purified by silica gel column chromatography with 0-10% methanol in dichloromethane to give partially clean product. The impure product was re-purified using a reverse phase HPLC-MS method using a Luna C₁₈(2) column (75×30 mm, 5 m particle size) sold by Phenomenex (pn: 00C-4252-U0-AX), and a dual gradient run from 1-99% mobile phase B over 15.0 minutes. Mobile phase A=H₂O (5 mM HCl). Mobile phase B=CH3CN. Flow rate=50 mL/min, and column temperature=25° C. to give 3-[[4-[(2R)-2-(tert-butoxycarbonylamino)-3-cyclopropyl-propoxy]-6-(2,6-dimethylphenyl)pyrimidin-2-yl]sulfamoyl]benzoic acid (45 mg, 18%) obtained as a colorless solid. ESI-MS m/z calc. 596.23047, found 597.3 (M+1)⁺; Retention time: 0.68 minutes; LC method D.

### Step 3: 3-[[4-[(2R)-2-Amino-3-cyclopropyl-propoxy]-6-(2,6-dimethylphenyl)pyrimidin-2-yl]sulfamoyl]benzoic Acid

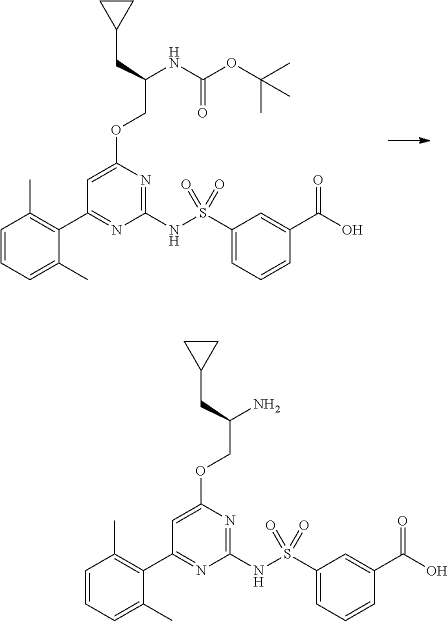 [SMILES: Cc1cccc(C)c1-c1cc(OC[C@@H](CC2CC2)NC(=O)OC(C)(C)C)nc(NS(=O)(=O)c2cccc(C(=O)O)c2)n1 ; Cc1cccc(C)c1-c1cc(OC[C@H](N)CC2CC2)nc(NS(=O)(=O)c2cccc(C(=O)O)c2)n1]

A solution of 3-[[4-[(2R)-2-(tert-butoxycarbonylamino)-3-cyclopropyl-propoxy]-6-(2,6-dimethylphenyl)pyrimidin-2-yl]sulfamoyl]benzoic acid (45 mg, 0.07542 mmol) in HCl (3 mL of 4 M, 12.00 mmol) (in dioxane) was stirred for four hours. The solvent was removed under vacuum, and the resulting solids were triturated with diethyl ether and dried under vacuum to give 3-[[4-[(2R)-2-amino-3-cyclopropyl-propoxy]-6-(2,6-dimethylphenyl)pyrimidin-2-yl]sulfamoyl]benzoic acid (hydrochloride salt) (53 mg, 132%). ESI-MS m/z calc. 496.17804, found 497.3 (M+1)⁺; Retention time: 0.41 minutes; LC method D.

### Step 4: (11R)-11-(Cyclopropylmethyl)-6-(2,6-dimethylphenyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 114)

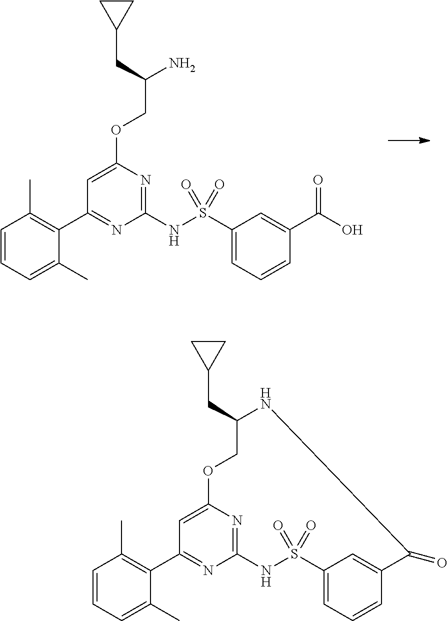 [SMILES: Cc1cccc(C)c1-c1cc(OC[C@H](N)CC2CC2)nc(NS(=O)(=O)c2cccc(C(=O)O)c2)n1 ; Cc1cccc(C)c1-c1cc2nc(n1)NS(=O)(=O)c1cccc(c1)C(=O)N[C@H](CC1CC1)CO2]

A solution of 3-[[4-[(2R)-2-amino-3-cyclopropyl-propoxy]-6-(2,6-dimethylphenyl)pyrimidin-2-yl]sulfamoyl]benzoic acid (hydrochloride salt) (40 mg, 0.07504 mmol), HATU (35 mg, 0.09205 mmol), and triethylamine (53 μL, 0.3803 mmol) in DMF (4 mL) was stirred for 17 hours. The reaction was concentrated under vacuum, filtered, and purified using a reverse phase HPLC-MS method using a Luna C₁₈ (2) column (75×30 mm, 5 m particle size) sold by Phenomenex (pn: 00C-4252-U0-AX), and a dual gradient run from 1-99% mobile phase B over 15.0 minutes. Mobile phase A=H₂O (5 mM HCl). Mobile phase B=CH3CN. Flow rate=50 mL/min, and column temperature=25° C. to give (11R)-11-(cyclopropylmethyl)-6-(2,6-dimethylphenyl)-9-oxa-2λ⁶-thia-3,5,12,19-tetraazatricyclo[12.3.1.14,8]nonadeca-1(17),4(19),5,7,14(18),15-hexaene-2,2,13-trione as a tan solid (22 mg, 61%) ESI-MS m/z calc. 478.16748, found 479.3 (M+1)⁺; Retention time: 1.38 minutes (LC method A). ¹H NMR (400 MHz, DMSO-d₆) δ 8.49 (s, 1H), 7.92 (s, 1H), 7.86 (d, J 10.0 Hz, 1H), 7.67 (s, 2H), 7.26 (t, J 7.6 Hz, 1H), 7.12 (d, J 7.6 Hz, 2H), 6.35 (s, 1H), 5.13 (dd, J 11.0, 3.7 Hz, 1H), 3.90 (t, J 11.2 Hz, 1H), 3.44-3.33 (m, 1H), 2.03 (s, 6H), 1.51 (td, J 14.6, 12.2, 7.7 Hz, 1H), 1.22 (dd, J 13.9, 7.2 Hz, 1H), 0.64-0.48 (m, 1H), 0.42-0.29 (m, 1H), 0.21-0.11 (m, 1H), 0.11-0.02 (m, 1H), −0.49 (s, 1H)

### VI. Characterization of New Compounds

The compounds in the following tables were prepared in a manner analogous to that described above using commercially available reagents and intermediates described herein.

TABLE 3
LCMS characterization data

| | | LCMS | | | |
|---|---|---|---|---|---|
| Compound | | Rt | Calc. | | LCMS |
| Number | Structure | (min) | Mass | M + 1 | Method |
| 115 | 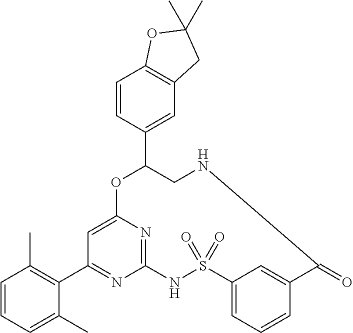 [SMILES: Cc1cccc(C)c1-c1cc2nc(n1)NS(=O)(=O)c1cccc(c1)C(=O)NCC(c1ccc3c(c1)CC(C)(C)O3)O2] | 1.68 | 570.194 | 571.3 | A |
| 116 | 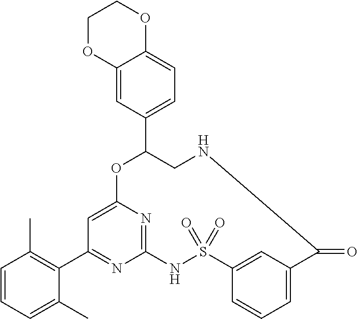 [SMILES: Cc1cccc(C)c1-c1cc2nc(n1)NS(=O)(=O)c1cccc(c1)C(=O)NCC(c1ccc3c(c1)OCCO3)O2] | 1.46 | 558.157 | 559.27 | A |
| 117 | 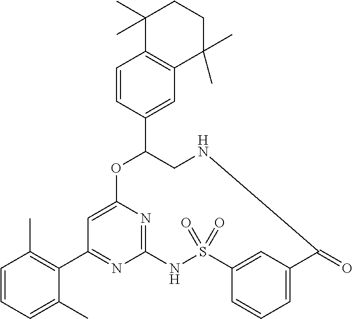 [SMILES: Cc1cccc(C)c1-c1cc2nc(n1)NS(=O)(=O)c1cccc(c1)C(=O)NCC(c1ccc3c(c1)C(C)(C)CCC3(C)C)O2] | 2.12 | 610.261 | 611.35 | A |
| 118 | 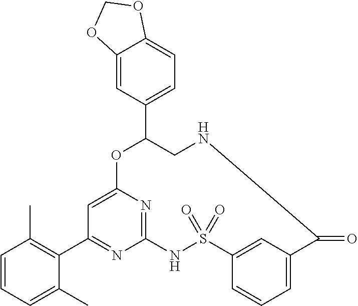 [SMILES: Cc1cccc(C)c1-c1cc2nc(n1)NS(=O)(=O)c1cccc(c1)C(=O)NCC(c1ccc3c(c1)OCO3)O2] | 1.42 | 544.142 | 545.4 | A |
| 119 | 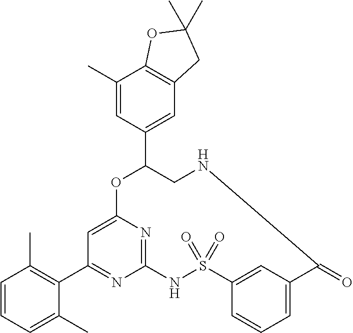 [SMILES: Cc1cc(C2CNC(=O)c3cccc(c3)S(=O)(=O)Nc3nc(cc(-c4c(C)cccc4C)n3)O2)cc2c1OC(C)(C)C2] | 1.43 | 584.209 | 585.35 | A |
| 33 | 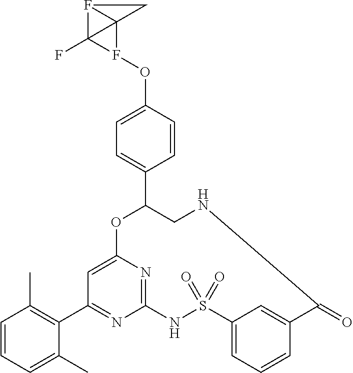 | 1.8 | 638.181 | 639.1 | A |
| 120 | 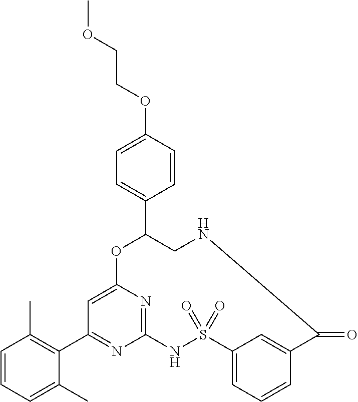 [SMILES: COCCOc1ccc(C2CNC(=O)c3cccc(c3)S(=O)(=O)Nc3nc(cc(-c4c(C)cccc4C)n3)O2)cc1] | 1.23 | 574.189 | 575.34 | A |
| 121 | 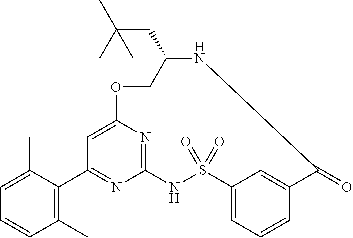 [SMILES: Cc1cccc(C)c1-c1cc2nc(n1)NS(=O)(=O)c1cccc(c1)C(=O)N[C@@H](CC(C)(C)C)CO2] | 2.27 | 494.199 | 495.1 | T |
| 122 | 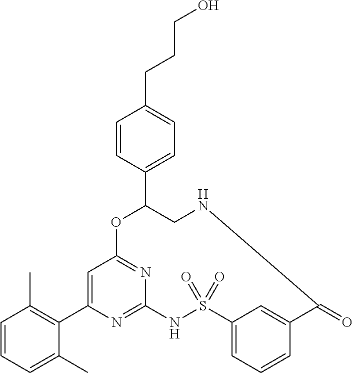 [SMILES: Cc1cccc(C)c1-c1cc2nc(n1)NS(=O)(=O)c1cccc(c1)C(=O)NCC(c1ccc(CCCO)cc1)O2] | 1.87 | 558.194 | 559.3 | T |
| 123 | 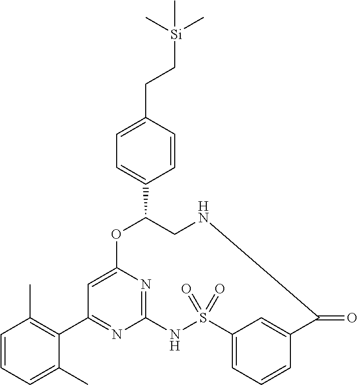 [SMILES: Cc1cccc(C)c1-c1cc2nc(n1)NS(=O)(=O)c1cccc(c1)C(=O)NC[C@@H](c1ccc(CC[Si](C)(C)C)cc1)O2] | 2.16 | 600.223 | 601.4 | A |
| 124 | 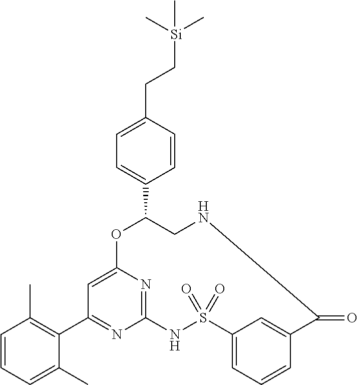 [SMILES: Cc1cccc(C)c1-c1cc2nc(n1)NS(=O)(=O)c1cccc(c1)C(=O)NC[C@@H](c1ccc(CC[Si](C)(C)C)cc1)O2] | 2.16 | 600.223 | 601.4 | A |
| 125 | 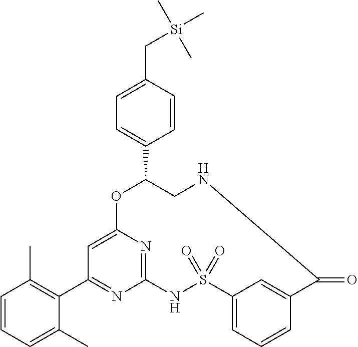 [SMILES: Cc1cccc(C)c1-c1cc2nc(n1)NS(=O)(=O)c1cccc(c1)C(=O)NC[C@@H](c1ccc(C[Si](C)(C)C)cc1)O2] | 2.03 | 586.207 | 587.5 | A |
| 126 | 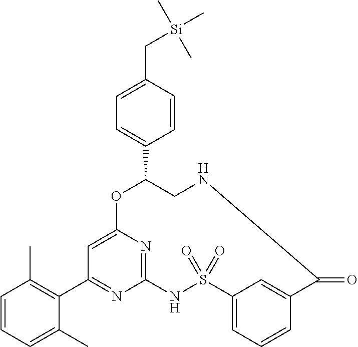 [SMILES: Cc1cccc(C)c1-c1cc2nc(n1)NS(=O)(=O)c1cccc(c1)C(=O)NC[C@@H](c1ccc(C[Si](C)(C)C)cc1)O2] | 2.03 | 586.207 | 587.5 | A |
| 127 | 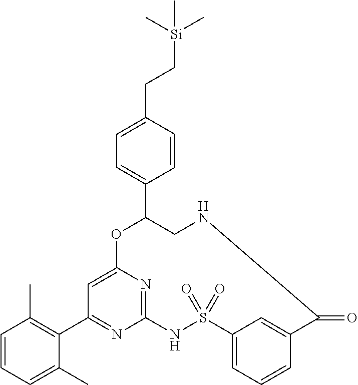 [SMILES: Cc1cccc(C)c1-c1cc2nc(n1)NS(=O)(=O)c1cccc(c1)C(=O)NCC(c1ccc(CC[Si](C)(C)C)cc1)O2] | 2.17 | 600.223 | 601.5 | A |
| 128 | 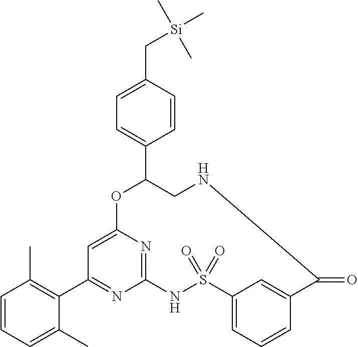 [SMILES: Cc1cccc(C)c1-c1cc2nc(n1)NS(=O)(=O)c1cccc(c1)C(=O)NCC(c1ccc(C[Si](C)(C)C)cc1)O2] | 2.03 | 586.207 | 587.4 | A |
| 129 | 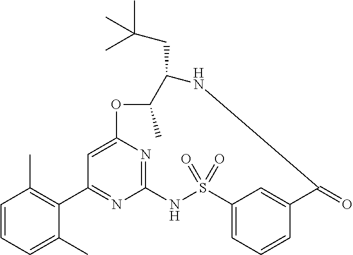 [SMILES: Cc1cccc(C)c1-c1cc2nc(n1)NS(=O)(=O)c1cccc(c1)C(=O)N[C@@H](CC(C)(C)C)[C@H](C)O2] | 1.64 | 508.214 | 509.4 | A |
| 130 | 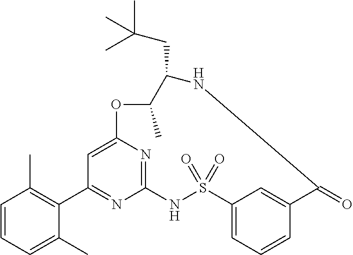 [SMILES: Cc1cccc(C)c1-c1cc2nc(n1)NS(=O)(=O)c1cccc(c1)C(=O)N[C@@H](CC(C)(C)C)[C@H](C)O2] | 1.62 | 508.214 | 509.34 | A |
| 131 | 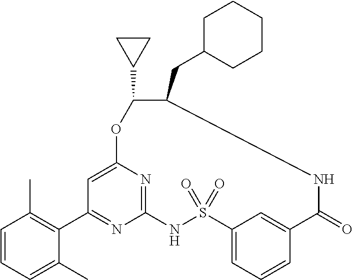 [SMILES: Cc1cccc(C)c1-c1cc2nc(n1)NS(=O)(=O)c1cccc(c1)C(=O)N[C@H](CC1CCCCC1)[C@@H](C1CC1)O2] | 1.88 | 560.246 | 561.4 | A |
| 132 | 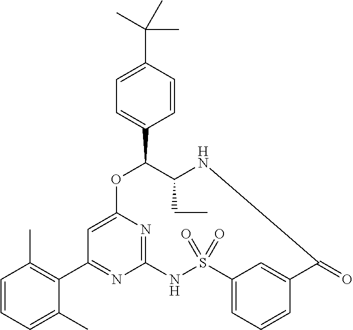 [SMILES: CC[C@H]1NC(=O)c2cccc(c2)S(=O)(=O)Nc2nc(cc(-c3c(C)cccc3C)n2)O[C@H]1c1ccc(C(C)(C)C)cc1] | 1.99 | 584.246 | 585.4 | A |
| 133 | 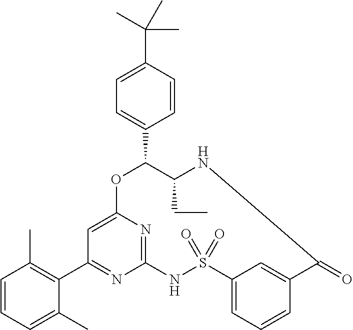 [SMILES: CC[C@H]1NC(=O)c2cccc(c2)S(=O)(=O)Nc2nc(cc(-c3c(C)cccc3C)n2)O[C@@H]1c1ccc(C(C)(C)C)cc1] | 2.05 | 584.246 | 585.4 | A |
| 134 | 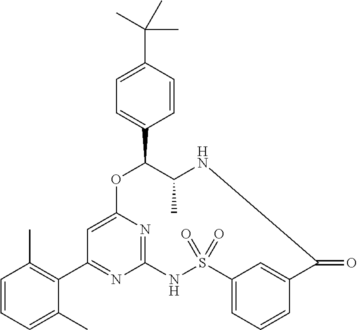 [SMILES: Cc1cccc(C)c1-c1cc2nc(n1)NS(=O)(=O)c1cccc(c1)C(=O)N[C@H](C)[C@H](c1ccc(C(C)(C)C)cc1)O2] | 1.92 | 570.23 | 571.4 | A |
| 135 | 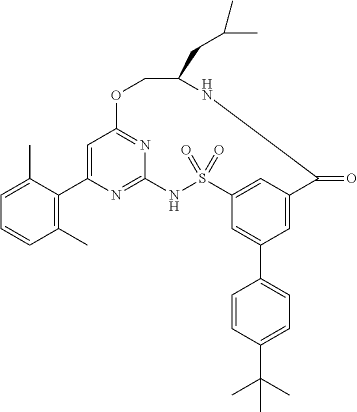 [SMILES: Cc1cccc(C)c1-c1cc2nc(n1)NS(=O)(=O)c1cc(cc(-c3ccc(C(C)(C)C)cc3)c1)C(=O)N[C@H](CC(C)C)CO2] | 2.16 | 612.277 | 613.4 | A |
| 136 | 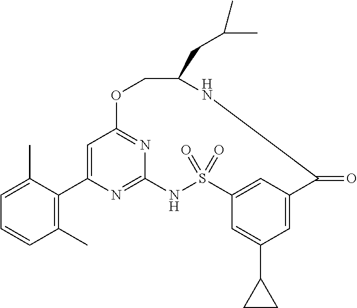 [SMILES: Cc1cccc(C)c1-c1cc2nc(n1)NS(=O)(=O)c1cc(cc(C3CC3)c1)C(=O)N[C@H](CC(C)C)CO2] | 1.7 | 520.214 | 521.4 | A |
| 137 | 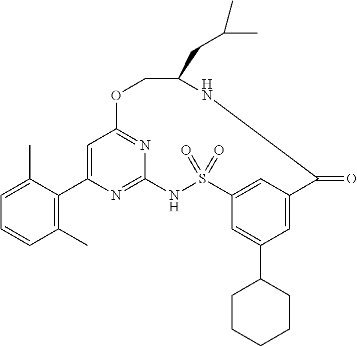 [SMILES: Cc1cccc(C)c1-c1cc2nc(n1)NS(=O)(=O)c1cc(cc(C3CCCCC3)c1)C(=O)N[C@H](CC(C)C)CO2] | 1.98 | 562.261 | 563.4 | A |
| 138 | 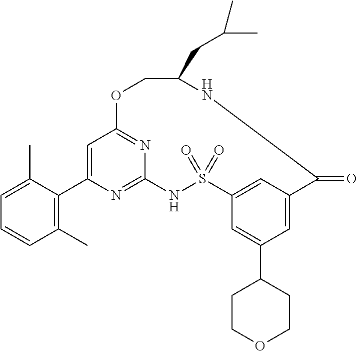 [SMILES: Cc1cccc(C)c1-c1cc2nc(n1)NS(=O)(=O)c1cc(cc(C3CCOCC3)c1)C(=O)N[C@H](CC(C)C)CO2] | 1.59 | 564.241 | 565.4 | A |
| 139 | 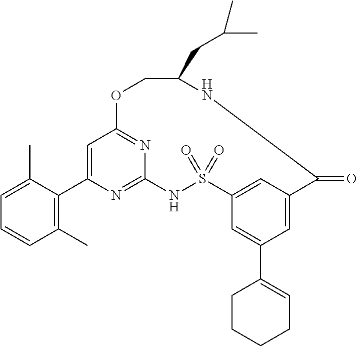 [SMILES: Cc1cccc(C)c1-c1cc2nc(n1)NS(=O)(=O)c1cc(cc(C3=CCCCC3)c1)C(=O)N[C@H](CC(C)C)CO2] | 1.95 | 560.246 | 561.4 | A |
| 140 | 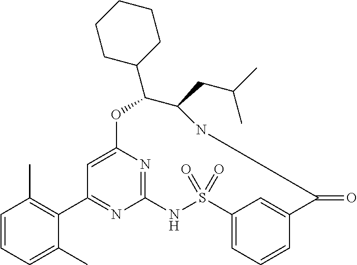 [SMILES: Cc1cccc(C)c1-c1cc2nc(n1)NS(=O)(=O)c1cccc(c1)C(=O)N[C@H](CC(C)C)[C@@H](C1CCCCC1)O2] | 1.95 | 562.261 | 563.1 | A |
| 141 | 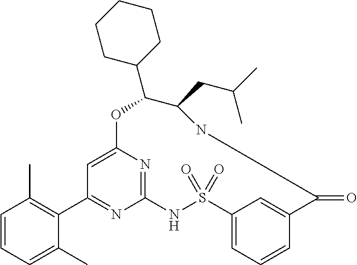 [SMILES: Cc1cccc(C)c1-c1cc2nc(n1)NS(=O)(=O)c1cccc(c1)C(=O)N[C@H](CC(C)C)[C@@H](C1CCCCC1)O2] | 1.99 | 562.261 | 563.1 | A |
| 142 | 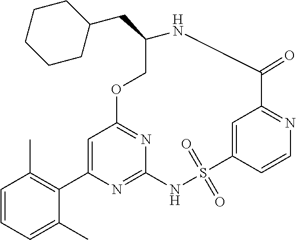 [SMILES: Cc1cccc(C)c1-c1cc2nc(n1)NS(=O)(=O)c1ccnc(c1)C(=O)N[C@H](CC1CCCCC1)CO2] | 1.58 | 521.21 | 522.3 | A |
| 143 | 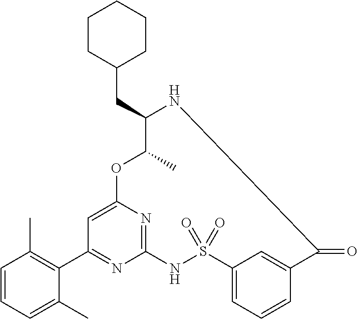 [SMILES: Cc1cccc(C)c1-c1cc2nc(n1)NS(=O)(=O)c1cccc(c1)C(=O)N[C@H](CC1CCCCC1)[C@H](C)O2] | 1.69 | 534.23 | 535.3 | A |
| 144 | 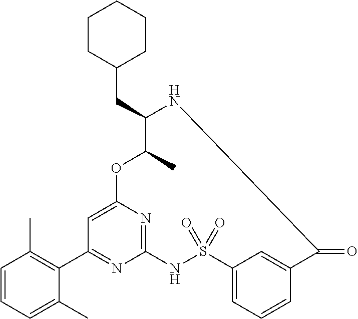 [SMILES: Cc1cccc(C)c1-c1cc2nc(n1)NS(=O)(=O)c1cccc(c1)C(=O)N[C@H](CC1CCCCC1)[C@@H](C)O2] | 1.75 | 534.23 | 535.3 | A |
| 145 | 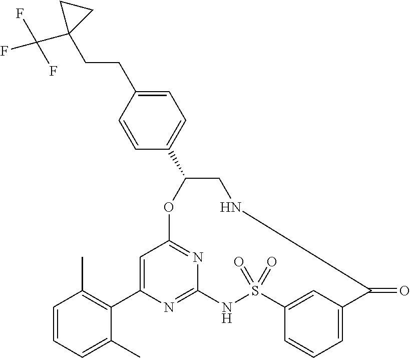 [SMILES: Cc1cccc(C)c1-c1cc2nc(n1)NS(=O)(=O)c1cccc(c1)C(=O)NC[C@@H](c1ccc(CCC3(C(F)(F)F)CC3)cc1)O2] | 1.73 | 636.202 | 637.46 | A |
| 146 | 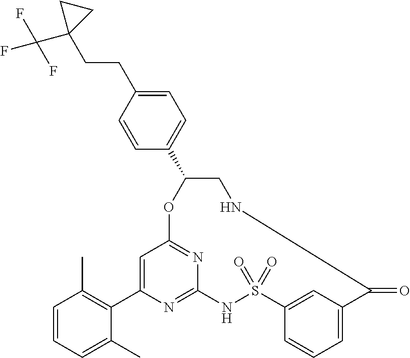 [SMILES: Cc1cccc(C)c1-c1cc2nc(n1)NS(=O)(=O)c1cccc(c1)C(=O)NC[C@@H](c1ccc(CCC3(C(F)(F)F)CC3)cc1)O2] | 1.73 | 636.202 | 637.46 | A |
| 147 | 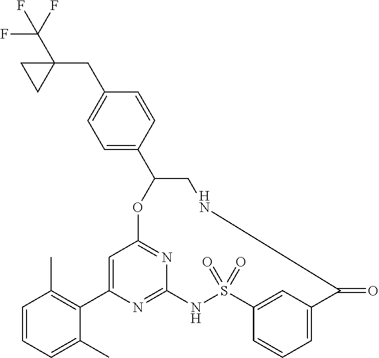 [SMILES: Cc1cccc(C)c1-c1cc2nc(n1)NS(=O)(=O)c1cccc(c1)C(=O)NCC(c1ccc(CC3(C(F)(F)F)CC3)cc1)O2] | 1.87 | 622.186 | 623.38 | A |
| 148 | 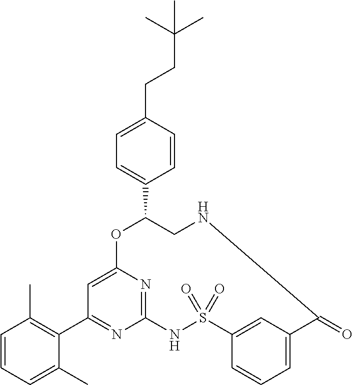 [SMILES: Cc1cccc(C)c1-c1cc2nc(n1)NS(=O)(=O)c1cccc(c1)C(=O)NC[C@@H](c1ccc(CCC(C)(C)C)cc1)O2] | 2.12 | 584.246 | 585.41 | A |
| 149 | 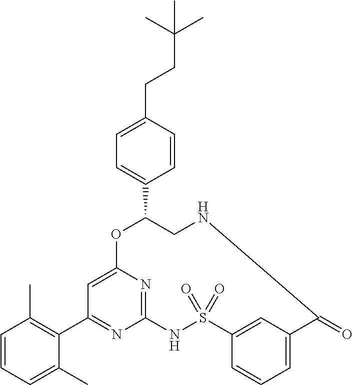 [SMILES: Cc1cccc(C)c1-c1cc2nc(n1)NS(=O)(=O)c1cccc(c1)C(=O)NC[C@@H](c1ccc(CCC(C)(C)C)cc1)O2] | 2.12 | 584.246 | 585.41 | A |
| 150 | 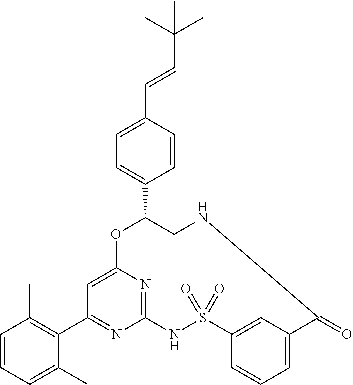 [SMILES: Cc1cccc(C)c1-c1cc2nc(n1)NS(=O)(=O)c1cccc(c1)C(=O)NC[C@@H](c1ccc(/C=C/C(C)(C)C)cc1)O2] | 0.79 | 582.23 | 583.43 | A |
| 151 | 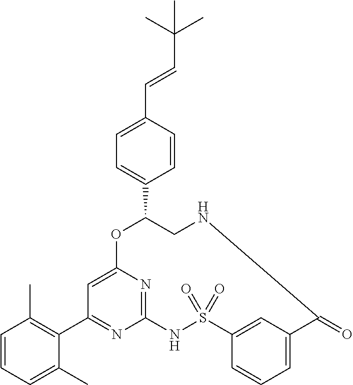 [SMILES: Cc1cccc(C)c1-c1cc2nc(n1)NS(=O)(=O)c1cccc(c1)C(=O)NC[C@@H](c1ccc(/C=C/C(C)(C)C)cc1)O2] | 0.79 | 582.23 | 583.43 | A |
| 54 | 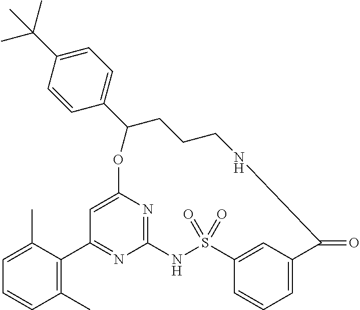 [SMILES: Cc1cccc(C)c1-c1cc2nc(n1)NS(=O)(=O)c1cccc(c1)C(=O)NCCCC(c1ccc(C(C)(C)C)cc1)O2] | 1.99 | 584.246 | 585.48 | A |
| 55 | 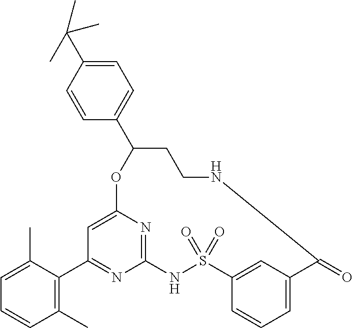 [SMILES: Cc1cccc(C)c1-c1cc2nc(n1)NS(=O)(=O)c1cccc(c1)C(=O)NCCC(c1ccc(C(C)(C)C)cc1)O2] | 1.93 | 570.23 | 571.43 | A |
| 152 | 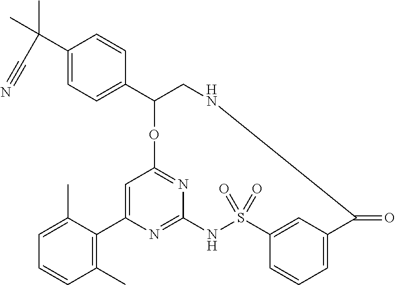 [SMILES: Cc1cccc(C)c1-c1cc2nc(n1)NS(=O)(=O)c1cccc(c1)C(=O)NCC(c1ccc(C(C)(C)C#N)cc1)O2] | 1.5 | 567.194 | 568.33 | A |
| 153 | 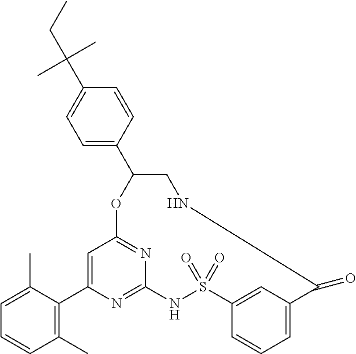 [SMILES: CCC(C)(C)c1ccc(C2CNC(=O)c3cccc(c3)S(=O)(=O)Nc3nc(cc(-c4c(C)cccc4C)n3)O2)cc1] | 2.61 | 570.23 | 571 | I |
| 154 | 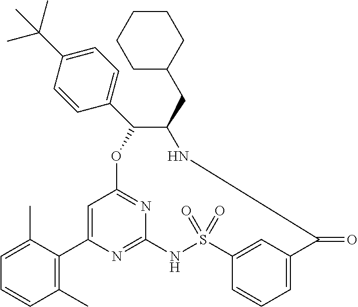 | 2.31 | 652.308 | 653.41 | A |
| 21 | 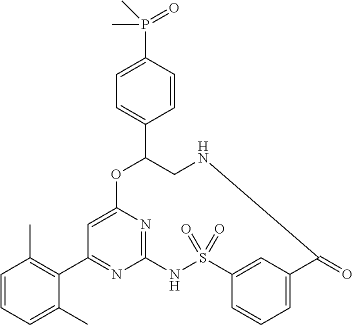 [SMILES: Cc1cccc(C)c1-c1cc2nc(n1)NS(=O)(=O)c1cccc(c1)C(=O)NCC(c1ccc(P(C)(C)=O)cc1)O2] | 0.98 | 576.16 | 577.5 | A |
| 155 | 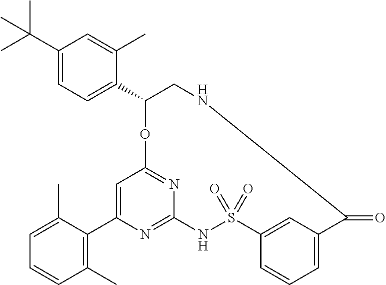 [SMILES: Cc1cc(C(C)(C)C)ccc1[C@@H]1CNC(=O)c2cccc(c2)S(=O)(=O)Nc2nc(cc(-c3c(C)cccc3C)n2)O1] | 1.94 | 570.23 | 571.38 | A |
| 156 | 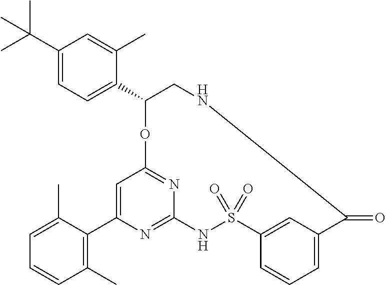 [SMILES: Cc1cc(C(C)(C)C)ccc1[C@@H]1CNC(=O)c2cccc(c2)S(=O)(=O)Nc2nc(cc(-c3c(C)cccc3C)n2)O1] | 1.93 | 570.23 | 571.38 | A |
| 157 | 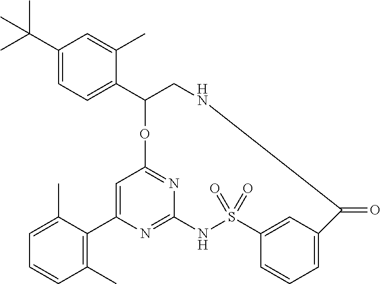 [SMILES: Cc1cc(C(C)(C)C)ccc1C1CNC(=O)c2cccc(c2)S(=O)(=O)Nc2nc(cc(-c3c(C)cccc3C)n2)O1] | 1.9 | 570.23 | 571.1 | A |
| 158 | 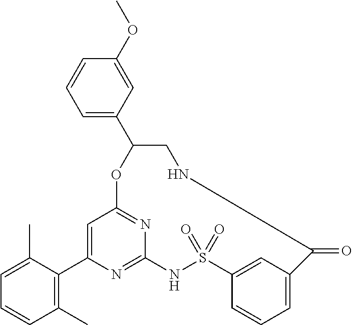 [SMILES: COc1cccc(C2CNC(=O)c3cccc(c3)S(=O)(=O)Nc3nc(cc(-c4c(C)cccc4C)n3)O2)c1] | 0.58 | 530.162 | 531.29 | A |
| 159 | 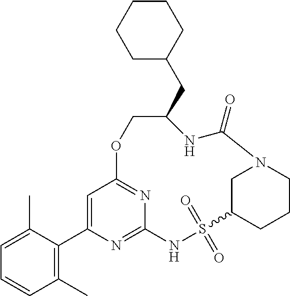 [SMILES: Cc1cccc(C)c1-c1cc2nc(n1)NS(=O)(=O)C1CCCN(C1)C(=O)N[C@H](CC1CCCCC1)CO2] | 1.49 | 527.257 | 528.36 | A |
| 160 | 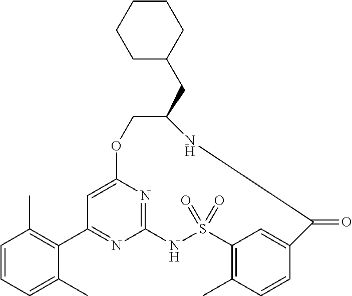 [SMILES: Cc1ccc2cc1S(=O)(=O)Nc1nc(cc(-c3c(C)cccc3C)n1)OC[C@@H](CC1CCCCC1)NC2=O] | 1.8 | 534.23 | 535.37 | A |
| 161 | 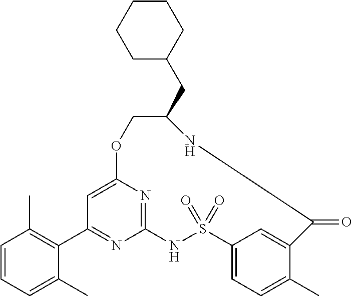 [SMILES: Cc1ccc2cc1C(=O)N[C@H](CC1CCCCC1)COc1cc(-c3c(C)cccc3C)nc(n1)NS2(=O)=O] | 1.7 | 534.23 | 535.37 | A |
| 162 | 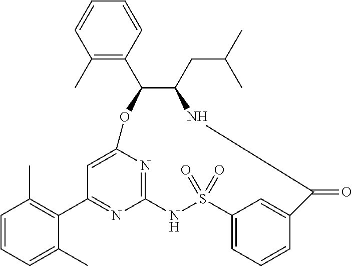 [SMILES: Cc1ccccc1[C@@H]1Oc2cc(-c3c(C)cccc3C)nc(n2)NS(=O)(=O)c2cccc(c2)C(=O)N[C@@H]1CC(C)C] | 1.97 | 570.23 | 571.3 | A |
| 163 | 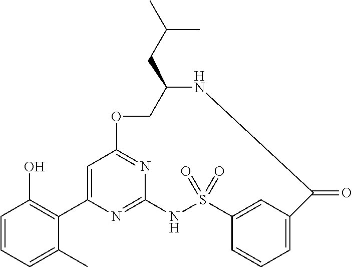 [SMILES: Cc1cccc(O)c1-c1cc2nc(n1)NS(=O)(=O)c1cccc(c1)C(=O)N[C@H](CC(C)C)CO2] | 1.81 | 482.162 | 483.4 | A |
| 164 | 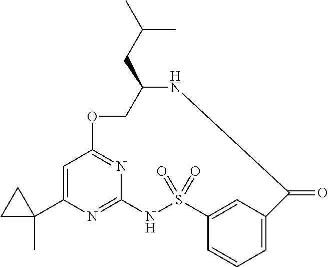 [SMILES: CC(C)C[C@@H]1COc2cc(C3(C)CC3)nc(n2)NS(=O)(=O)c2cccc(c2)C(=O)N1] | 0.99 | 430.167 | 431.06 | Q |
| 165 | 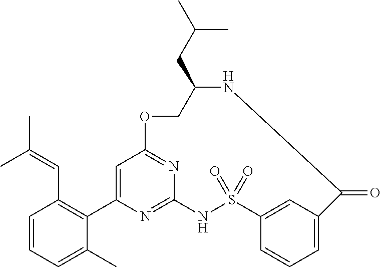 [SMILES: CC(C)=Cc1cccc(C)c1-c1cc2nc(n1)NS(=O)(=O)c1cccc(c1)C(=O)N[C@H](CC(C)C)CO2] | 1.35 | 520.214 | 521.4 | Q |
| 166 | 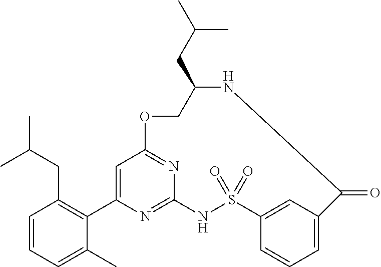 [SMILES: Cc1cccc(CC(C)C)c1-c1cc2nc(n1)NS(=O)(=O)c1cccc(c1)C(=O)N[C@H](CC(C)C)CO2] | 1.06 | 522.23 | 523.48 | Q |
| 167 | 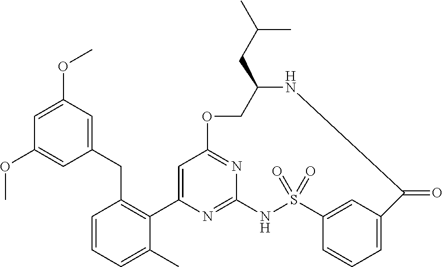 [SMILES: COc1cc(Cc2cccc(C)c2-c2cc3nc(n2)NS(=O)(=O)c2cccc(c2)C(=O)N[C@H](CC(C)C)CO3)cc(OC)c1] | 1.35 | 616.236 | 617.17 | Q |
| 168 | 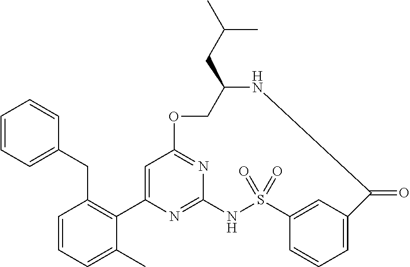 [SMILES: Cc1cccc(Cc2ccccc2)c1-c1cc2nc(n1)NS(=O)(=O)c1cccc(c1)C(=O)N[C@H](CC(C)C)CO2] | 1.32 | 556.214 | 557.08 | Q |
| 170 | 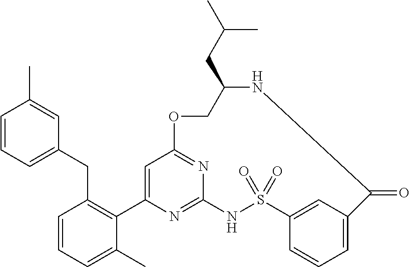 [SMILES: Cc1cccc(Cc2cccc(C)c2-c2cc3nc(n2)NS(=O)(=O)c2cccc(c2)C(=O)N[C@H](CC(C)C)CO3)c1] | 1.295 | 570.23 | 571.105 | Q |
| 171 | 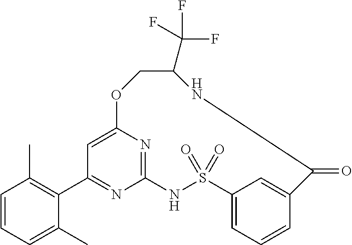 [SMILES: Cc1cccc(C)c1-c1cc2nc(n1)NS(=O)(=O)c1cccc(c1)C(=O)NC(C(F)(F)F)CO2] | 1.36 | 492.108 | 492.97 | A |
| 172 | 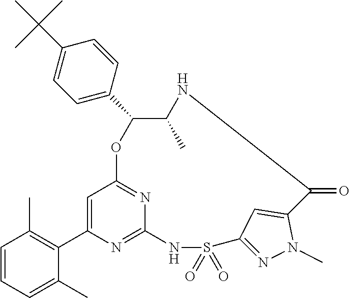 [SMILES: Cc1cccc(C)c1-c1cc2nc(n1)NS(=O)(=O)c1cc(n(C)n1)C(=O)N[C@H](C)[C@@H](c1ccc(C(C)(C)C)cc1)O2] | 1.84 | 574.236 | 575.4 | A |
| 173 | 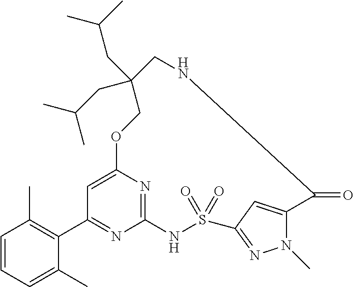 [SMILES: Cc1cccc(C)c1-c1cc2nc(n1)NS(=O)(=O)c1cc(n(C)n1)C(=O)NCC(CC(C)C)(CC(C)C)CO2] | 1.89 | 554.268 | 555.6 | A |
| 76 | 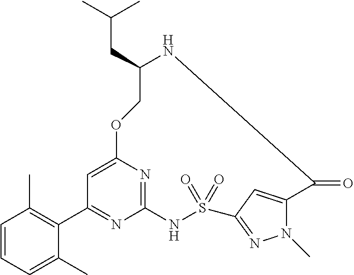 [SMILES: Cc1cccc(C)c1-c1cc2nc(n1)NS(=O)(=O)c1cc(n(C)n1)C(=O)N[C@H](CC(C)C)CO2] | 1.37 | 484.189 | 485.3 | A |
| 174 | 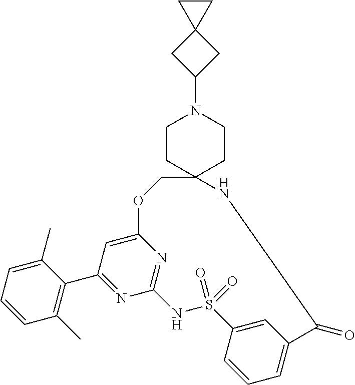 [SMILES: Cc1cccc(C)c1-c1cc2nc(n1)NS(=O)(=O)c1cccc(c1)C(=O)NC1(CCN(C3CC4(CC4)C3)CC1)CO2] | 0.98 | 573.241 | 574.4 | A |
| 175 | 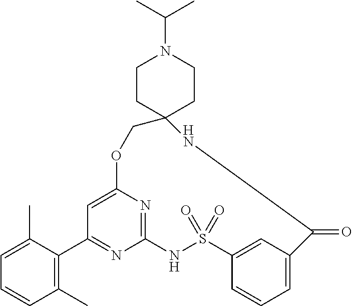 [SMILES: Cc1cccc(C)c1-c1cc2nc(n1)NS(=O)(=O)c1cccc(c1)C(=O)NC1(CCN(C(C)C)CC1)CO2] | 0.81 | 535.225 | 536.3 | A |
| 176 | 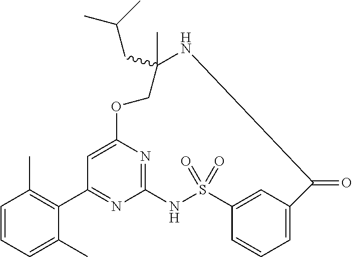 [SMILES: Cc1cccc(C)c1-c1cc2nc(n1)NS(=O)(=O)c1cccc(c1)C(=O)NC(C)(CC(C)C)CO2] | 1.61 | 494.199 | 495.3 | A |
| 177 | 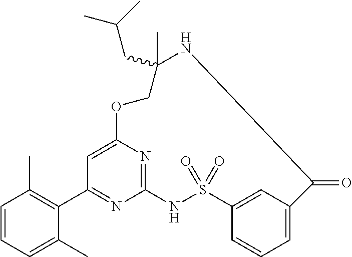 [SMILES: Cc1cccc(C)c1-c1cc2nc(n1)NS(=O)(=O)c1cccc(c1)C(=O)NC(C)(CC(C)C)CO2] | 1.61 | 494.199 | 495.4 | A |
| 178 | 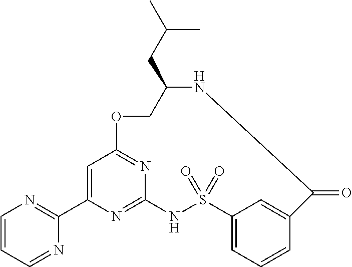 [SMILES: CC(C)C[C@@H]1COc2cc(-c3ncccn3)nc(n2)NS(=O)(=O)c2cccc(c2)C(=O)N1] | 1.09 | 454.142 | 455.2 | A |
| 179 | 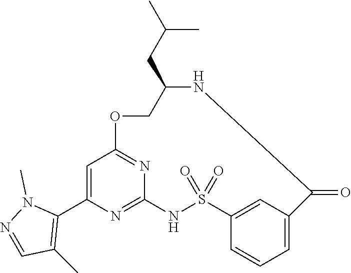 [SMILES: Cc1cnn(C)c1-c1cc2nc(n1)NS(=O)(=O)c1cccc(c1)C(=O)N[C@H](CC(C)C)CO2] | 1.29 | 470.174 | 471.2 | A |
| 180 | 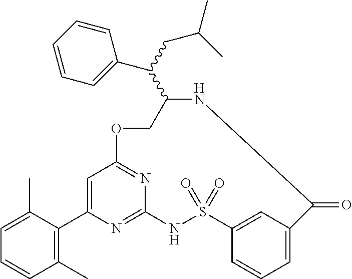 | 1.84 | 570.23 | 571.3 | A |
| 181 | 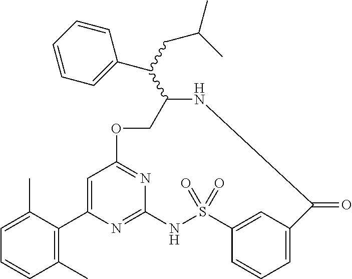 | 1.84 | 570.23 | 571.3 | A |
| 182 | 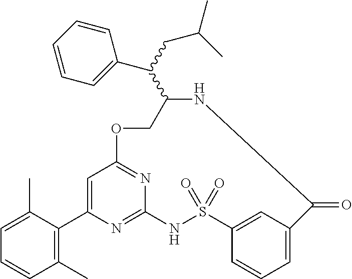 | 1.79 | 570.23 | 571.3 | A |
| 183 | 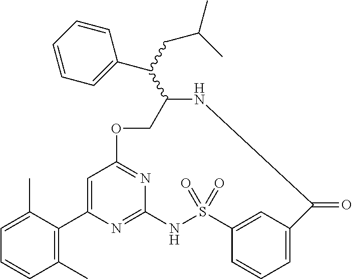 | 1.79 | 570.23 | 571.3 | A |
| 495 | 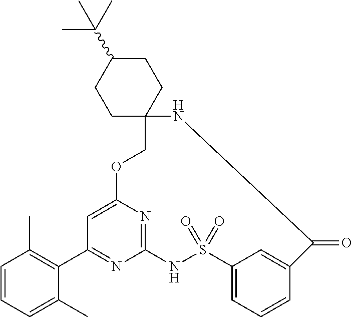 [SMILES: Cc1cccc(C)c1-c1cc2nc(n1)NS(=O)(=O)c1cccc(c1)C(=O)NC1(CCC(C(C)(C)C)CC1)CO2] | 1.83 | 548.246 | 549.4 | A |
| 184 | 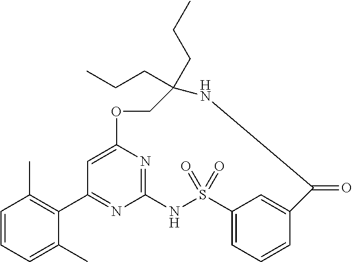 [SMILES: CCCC1(CCC)COc2cc(-c3c(C)cccc3C)nc(n2)NS(=O)(=O)c2cccc(c2)C(=O)N1] | 1.65 | 508.214 | 509.3 | A |
| 185 | 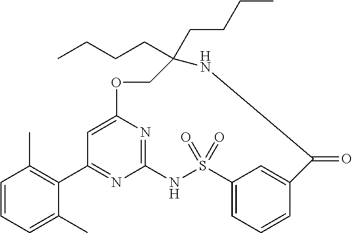 [SMILES: CCCCC1(CCCC)COc2cc(-c3c(C)cccc3C)nc(n2)NS(=O)(=O)c2cccc(c2)C(=O)N1] | 1.86 | 536.246 | 537.4 | A |
| 48 | 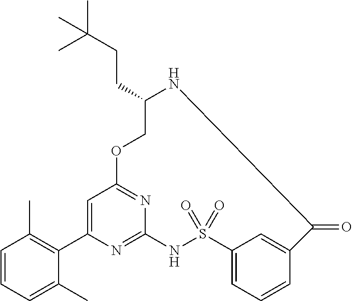 [SMILES: Cc1cccc(C)c1-c1cc2nc(n1)NS(=O)(=O)c1cccc(c1)C(=O)N[C@@H](CCC(C)(C)C)CO2] | 1.75 | 508.214 | 509.4 | A |
| 186 | 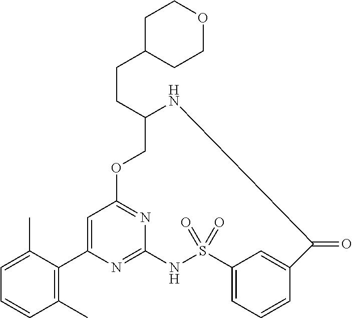 [SMILES: Cc1cccc(C)c1-c1cc2nc(n1)NS(=O)(=O)c1cccc(c1)C(=O)NC(CCC1CCOCC1)CO2] | 1.37 | 536.209 | 537.4 | A |
| 187 | 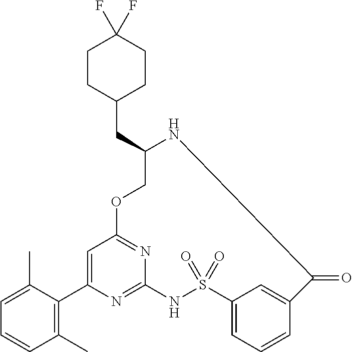 [SMILES: Cc1cccc(C)c1-c1cc2nc(n1)NS(=O)(=O)c1cccc(c1)C(=O)N[C@H](CC1CCC(F)(F)CC1)CO2] | 1.59 | 556.196 | 557.4 | A |
| 188 | 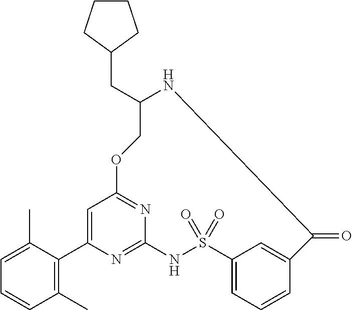 [SMILES: Cc1cccc(C)c1-c1cc2nc(n1)NS(=O)(=O)c1cccc(c1)C(=O)NC(CC1CCCC1)CO2] | 1.65 | 506.199 | 507.4 | A |
| 189 | 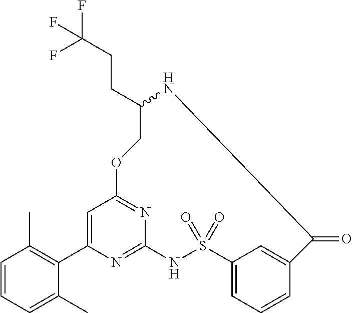 [SMILES: Cc1cccc(C)c1-c1cc2nc(n1)NS(=O)(=O)c1cccc(c1)C(=O)NC(CCC(F)(F)F)CO2] | 1.47 | 520.139 | 521.2 | A |
| 190 | 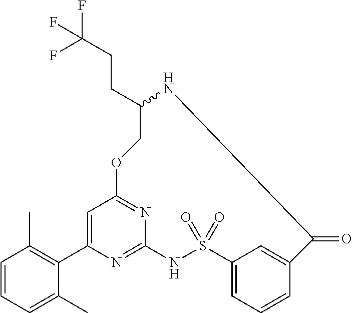 [SMILES: Cc1cccc(C)c1-c1cc2nc(n1)NS(=O)(=O)c1cccc(c1)C(=O)NC(CCC(F)(F)F)CO2] | 1.48 | 520.139 | 521.3 | A |
| 191 | 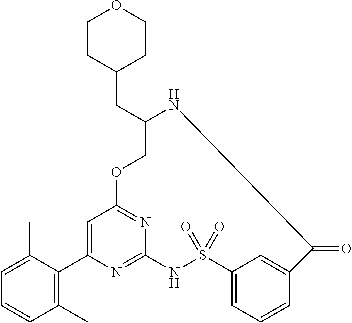 [SMILES: Cc1cccc(C)c1-c1cc2nc(n1)NS(=O)(=O)c1cccc(c1)C(=O)NC(CC1CCOCC1)CO2] | 1.3 | 522.194 | 523.4 | A |
| 29 | 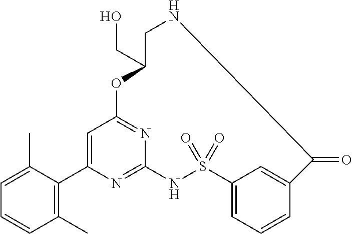 [SMILES: Cc1cccc(C)c1-c1cc2nc(n1)NS(=O)(=O)c1cccc(c1)C(=O)NC[C@H](CO)O2] | 0.93 | 454.131 | 455.3 | A |
| 28 | 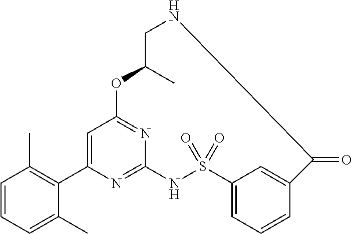 [SMILES: Cc1cccc(C)c1-c1cc2nc(n1)NS(=O)(=O)c1cccc(c1)C(=O)NC[C@@H](C)O2] | 1.15 | 438.136 | 439.2 | A |
| 27 | 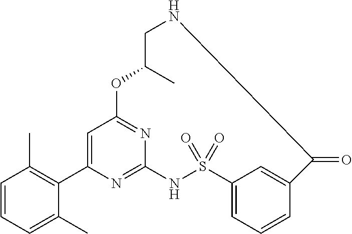 [SMILES: Cc1cccc(C)c1-c1cc2nc(n1)NS(=O)(=O)c1cccc(c1)C(=O)NC[C@H](C)O2] | 1.15 | 438.136 | 439.3 | A |
| 26 | 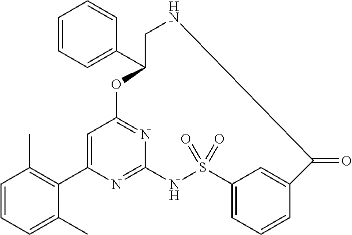 [SMILES: Cc1cccc(C)c1-c1cc2nc(n1)NS(=O)(=O)c1cccc(c1)C(=O)NC[C@H](c1ccccc1)O2] | 1.42 | 500.152 | 501.3 | A |
| 25 | 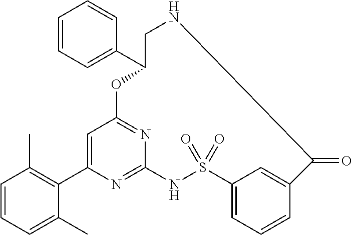 [SMILES: Cc1cccc(C)c1-c1cc2nc(n1)NS(=O)(=O)c1cccc(c1)C(=O)NC[C@@H](c1ccccc1)O2] | 1.42 | 500.152 | 501.3 | A |
| 192 | 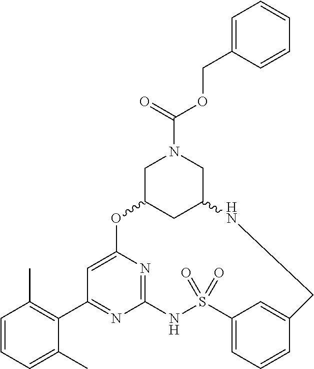 [SMILES: Cc1cccc(C)c1-c1cc2nc(n1)NS(=O)(=O)c1cccc(c1)CNC1CC(CN(C(=O)OCc3ccccc3)C1)O2] | 1.35 | 599.22 | 600.4 | A |
| 193 | 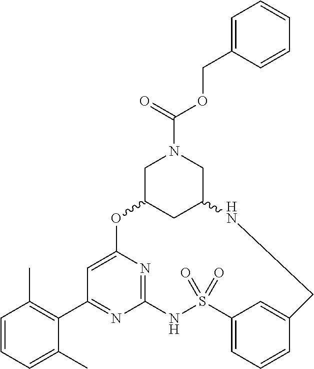 [SMILES: Cc1cccc(C)c1-c1cc2nc(n1)NS(=O)(=O)c1cccc(c1)CNC1CC(CN(C(=O)OCc3ccccc3)C1)O2] | 1.38 | 599.22 | 600.5 | A |
| 194 | 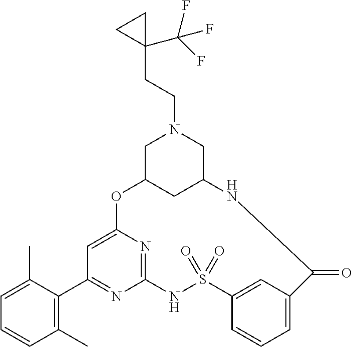 [SMILES: Cc1cccc(C)c1-c1cc2nc(n1)NS(=O)(=O)c1cccc(c1)C(=O)NC1CC(CN(CCC3(C(F)(F)F)CC3)C1)O2] | 1.17 | 615.213 | 616.3 | A |
| 195 | 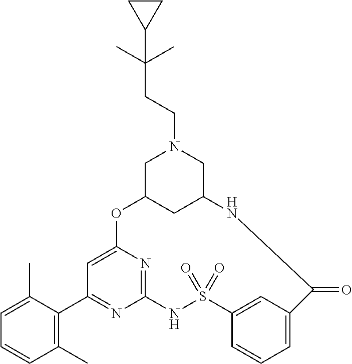 [SMILES: Cc1cccc(C)c1-c1cc2nc(n1)NS(=O)(=O)c1cccc(c1)C(=O)NC1CC(CN(CCC(C)(C)C3CC3)C1)O2] | 1.28 | 589.272 | 590.5 | A |
| 196 | 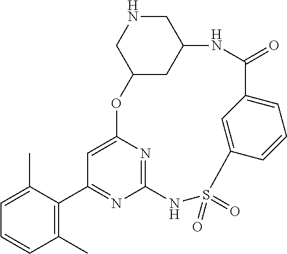 [SMILES: Cc1cccc(C)c1-c1cc2nc(n1)NS(=O)(=O)c1cccc(c1)C(=O)NC1CNCC(C1)O2] | 0.89 | 479.163 | 480.4 | A |
| 197 | 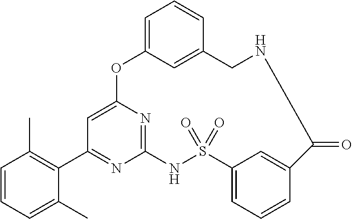 [SMILES: Cc1cccc(C)c1-c1cc2nc(n1)NS(=O)(=O)c1cccc(c1)C(=O)NCc1cccc(c1)O2] | 1.46 | 486.136 | 487.2 | A |
| 198 | 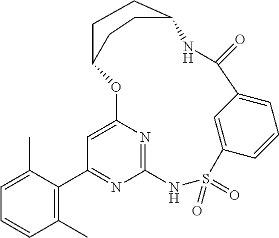 [SMILES: Cc1cccc(C)c1-c1cc2nc(n1)NS(=O)(=O)c1cccc(c1)C(=O)N[C@H]1CC[C@H](CC1)O2] | 1.4 | 478.167 | 479.3 | A |
| 199 | 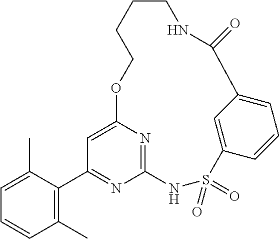 [SMILES: Cc1cccc(C)c1-c1cc2nc(n1)NS(=O)(=O)c1cccc(c1)C(=O)NCCCCO2] | 1.36 | 452.152 | 453.2 | A |
| 200 | 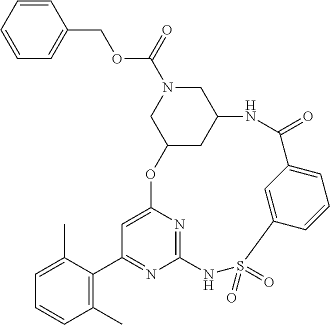 [SMILES: Cc1cccc(C)c1-c1cc2nc(n1)NS(=O)(=O)c1cccc(c1)C(=O)NC1CC(CN(C(=O)OCc3ccccc3)C1)O2] | 1.54 | 613.2 | 614.2 | A |
| 201 | 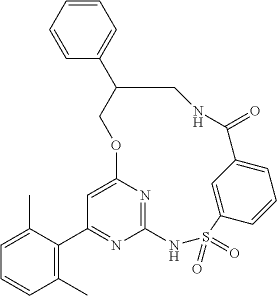 [SMILES: Cc1cccc(C)c1-c1cc2nc(n1)NS(=O)(=O)c1cccc(c1)C(=O)NCC(c1ccccc1)CO2] | 1.54 | 514.167 | 515.2 | A |
| 202 | 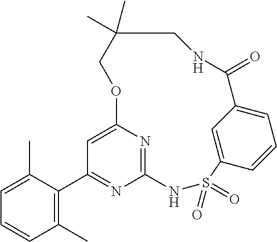 [SMILES: Cc1cccc(C)c1-c1cc2nc(n1)NS(=O)(=O)c1cccc(c1)C(=O)NCC(C)(C)CO2] | 1.36 | 466.167 | 467.3 | A |
| 203 | 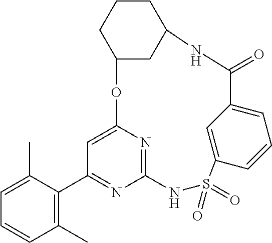 [SMILES: Cc1cccc(C)c1-c1cc2nc(n1)NS(=O)(=O)c1cccc(c1)C(=O)NC1CCCC(C1)O2] | 1.44 | 478.167 | 479.2 | A |
| 204 | 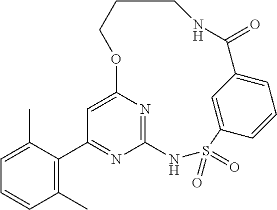 [SMILES: Cc1cccc(C)c1-c1cc2nc(n1)NS(=O)(=O)c1cccc(c1)C(=O)NCCCO2] | 1.2 | 438.136 | 439.2 | A |
| 205 | 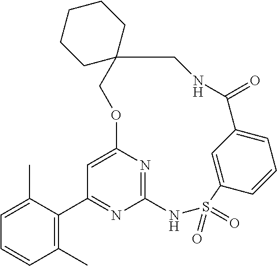 [SMILES: Cc1cccc(C)c1-c1cc2nc(n1)NS(=O)(=O)c1cccc(c1)C(=O)NCC1(CCCCC1)CO2] | 1.7 | 506.199 | 507.3 | A |
| 206 | 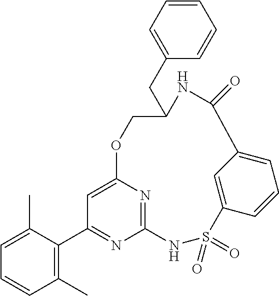 [SMILES: Cc1cccc(C)c1-c1cc2nc(n1)NS(=O)(=O)c1cccc(c1)C(=O)NC(Cc1ccccc1)CO2] | 1.55 | 514.167 | 515.2 | A |
| 207 | 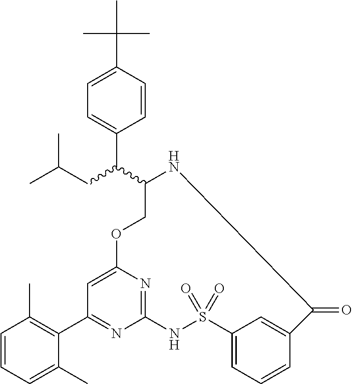 | 2.09 | 626.293 | 627.5 | A |
| 208 | 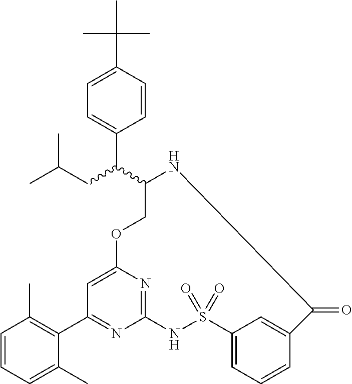 | 2.09 | 626.293 | 627.6 | A |
| 209 | 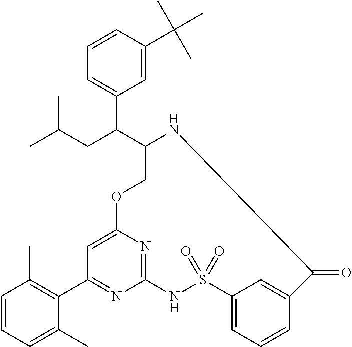 [SMILES: Cc1cccc(C)c1-c1cc2nc(n1)NS(=O)(=O)c1cccc(c1)C(=O)NC(C(CC(C)C)c1cccc(C(C)(C)C)c1)CO2] | 2.08 | 626.293 | 627.7 | A |
| 210 | 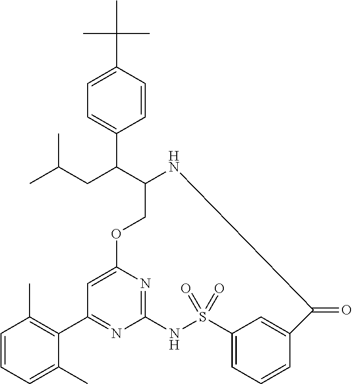 [SMILES: Cc1cccc(C)c1-c1cc2nc(n1)NS(=O)(=O)c1cccc(c1)C(=O)NC(C(CC(C)C)c1ccc(C(C)(C)C)cc1)CO2] | 2.09 | 626.293 | 627.6 | A |
| 211 | 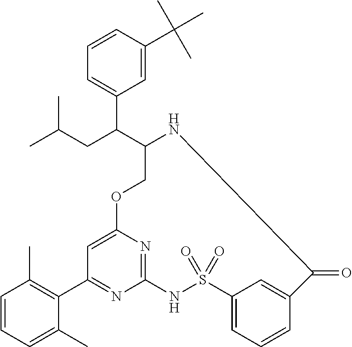 [SMILES: Cc1cccc(C)c1-c1cc2nc(n1)NS(=O)(=O)c1cccc(c1)C(=O)NC(C(CC(C)C)c1cccc(C(C)(C)C)c1)CO2] | 2.18 | 626.293 | 627.5 | A |
| 212 | 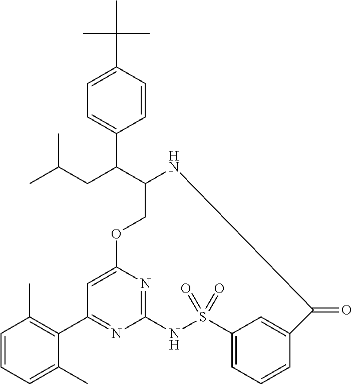 [SMILES: Cc1cccc(C)c1-c1cc2nc(n1)NS(=O)(=O)c1cccc(c1)C(=O)NC(C(CC(C)C)c1ccc(C(C)(C)C)cc1)CO2] | 2.2 | 626.293 | 627.5 | A |
| 213 | 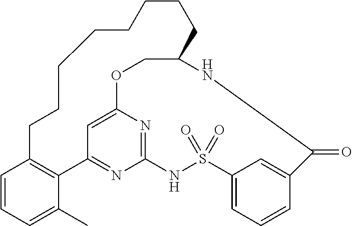 [SMILES: Cc1cccc2c1-c1cc3nc(n1)NS(=O)(=O)c1cccc(c1)C(=O)N[C@H](CCCCCCCC2)CO3] | 1.59 | 520.214 | 521.3 | A |
| 214 | 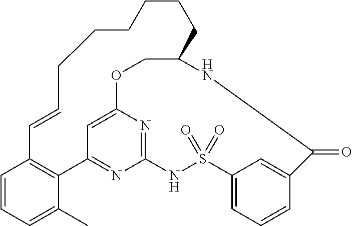 [SMILES: Cc1cccc2c1-c1cc3nc(n1)NS(=O)(=O)c1cccc(c1)C(=O)N[C@H](CCCCCC/C=C/2)CO3] | 1.56 | 518.199 | 519.4 | A |
| 215 | 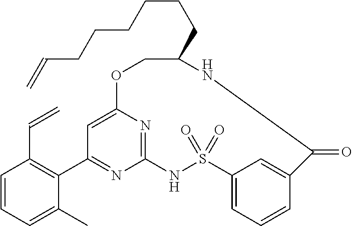 [SMILES: C=CCCCCCC[C@@H]1COc2cc(-c3c(C)cccc3C=C)nc(n2)NS(=O)(=O)c2cccc(c2)C(=O)N1] | 1.87 | 546.23 | 547.4 | A |
| 216 | 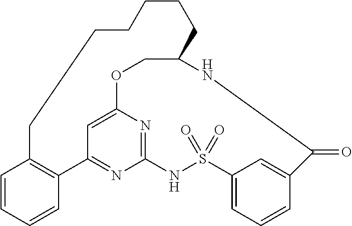 [SMILES: O=C1N[C@@H]2CCCCCCc3ccccc3-c3cc(nc(n3)NS(=O)(=O)c3cccc1c3)OC2] | 1.31 | 478.167 | 479.2 | A |
| 217 | 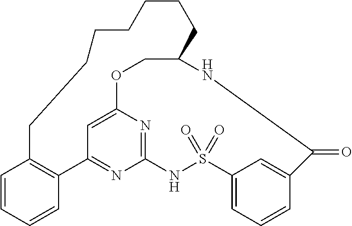 [SMILES: O=C1N[C@@H]2CCCCCCCc3ccccc3-c3cc(nc(n3)NS(=O)(=O)c3cccc1c3)OC2] | 1.42 | 492.183 | 493.2 | A |
| 218 | 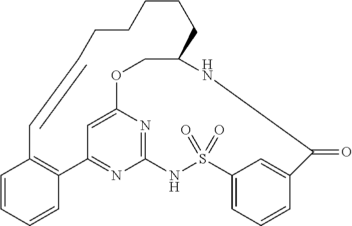 [SMILES: O=C1N[C@@H]2CCCCC/C=C/c3ccccc3-c3cc(nc(n3)NS(=O)(=O)c3cccc1c3)OC2] | 1.42 | 490.167 | 491.2 | A |
| 219 | 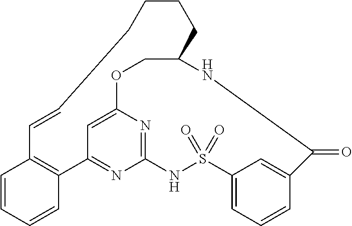 [SMILES: O=C1N[C@@H]2CCCC/C=C/c3ccccc3-c3cc(nc(n3)NS(=O)(=O)c3cccc1c3)OC2] | 1.31 | 476.152 | 477.2 | A |
| 35 | 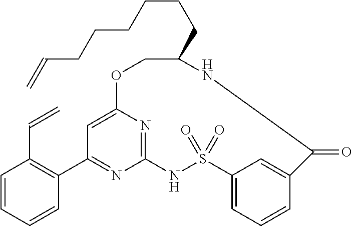 [SMILES: C=CCCCCCC[C@@H]1COc2cc(-c3ccccc3C=C)nc(n2)NS(=O)(=O)c2cccc(c2)C(=O)N1] | 1.86 | 532.214 | 533.3 | A |
| 220 | 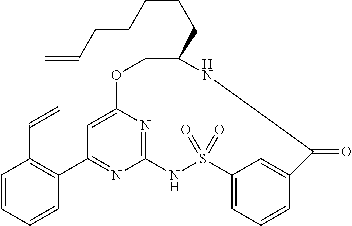 [SMILES: C=CCCCCC[C@@H]1COc2cc(-c3ccccc3C=C)nc(n2)NS(=O)(=O)c2cccc(c2)C(=O)N1] | 1.75 | 518.199 | 519.3 | A |
| 221 | 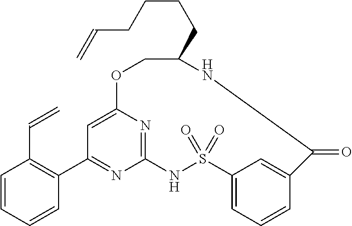 [SMILES: C=CCCCC[C@@H]1COc2cc(-c3ccccc3C=C)nc(n2)NS(=O)(=O)c2cccc(c2)C(=O)N1] | 1.65 | 504.183 | 505.3 | A |
| 222 | 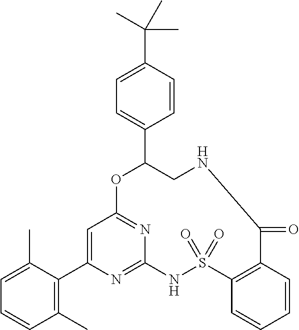 [SMILES: Cc1cccc(C)c1-c1cc2nc(n1)NS(=O)(=O)c1ccccc1C(=O)NCC(c1ccc(C(C)(C)C)cc1)O2] | 2 | 556.214 | 557.2 | A |
| 223 | 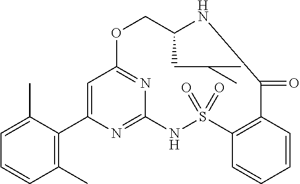 [SMILES: Cc1cccc(C)c1-c1cc2nc(n1)NS(=O)(=O)c1ccccc1C(=O)N[C@H](CC(C)C)CO2] | 1.71 | 480.183 | 481.2 | A |
| 224 | 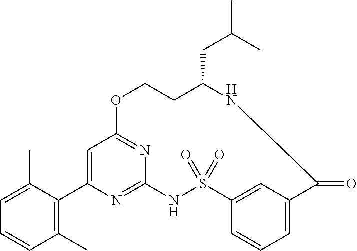 [SMILES: Cc1cccc(C)c1-c1cc2nc(n1)NS(=O)(=O)c1cccc(c1)C(=O)N[C@@H](CC(C)C)CCO2] | 1.55 | 494.199 | 495.3 | A |
| 225 | 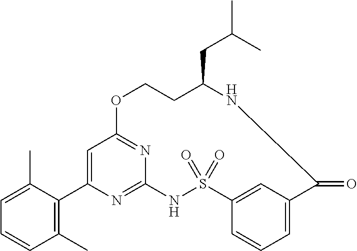 [SMILES: Cc1cccc(C)c1-c1cc2nc(n1)NS(=O)(=O)c1cccc(c1)C(=O)N[C@H](CC(C)C)CCO2] | 1.54 | 494.199 | 495.3 | A |
| 226 | 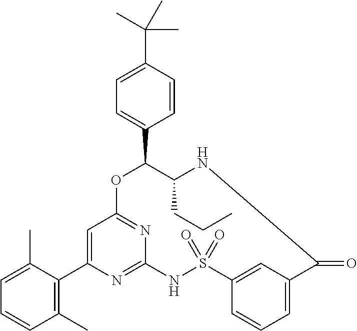 [SMILES: CCC[C@H]1NC(=O)c2cccc(c2)S(=O)(=O)Nc2nc(cc(-c3c(C)cccc3C)n2)O[C@H]1c1ccc(C(C)(C)C)cc1] | 1.99 | 598.261 | 599.5 | A |
| 227 | 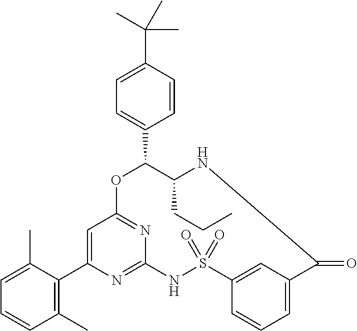 [SMILES: CCC[C@H]1NC(=O)c2cccc(c2)S(=O)(=O)Nc2nc(cc(-c3c(C)cccc3C)n2)O[C@@H]1c1ccc(C(C)(C)C)cc1] | 2.04 | 598.261 | 599.5 | A |
| 228 | 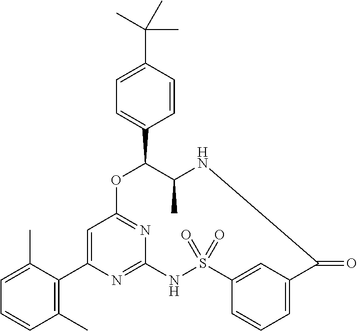 [SMILES: Cc1cccc(C)c1-c1cc2nc(n1)NS(=O)(=O)c1cccc(c1)C(=O)N[C@@H](C)[C@H](c1ccc(C(C)(C)C)cc1)O2] | 1.91 | 570.23 | 571.5 | A |
| 229 | 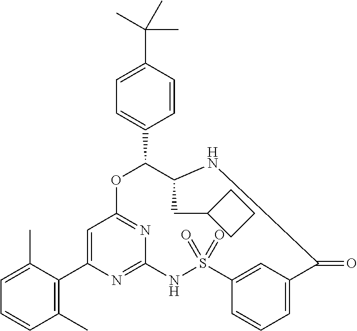 [SMILES: Cc1cccc(C)c1-c1cc2nc(n1)NS(=O)(=O)c1cccc(c1)C(=O)N[C@H](CC1CCC1)[C@@H](c1ccc(C(C)(C)C)cc1)O2] | 2.15 | 624.277 | 625.5 | A |
| 230 | 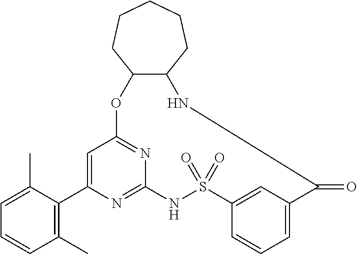 [SMILES: Cc1cccc(C)c1-c1cc2nc(n1)NS(=O)(=O)c1cccc(c1)C(=O)NC1CCCCCC1O2] | 1.53 | 492.183 | 493.4 | A |
| 231 | 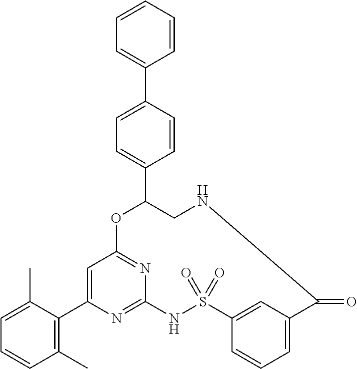 [SMILES: Cc1cccc(C)c1-c1cc2nc(n1)NS(=O)(=O)c1cccc(c1)C(=O)NCC(c1ccc(-c3ccccc3)cc1)O2] | 1.77 | 576.183 | 577.4 | A |
| 232 | 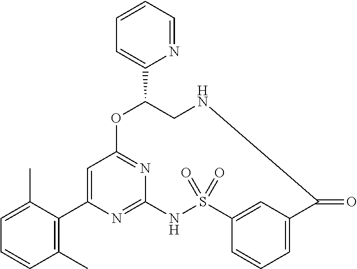 [SMILES: Cc1cccc(C)c1-c1cc2nc(n1)NS(=O)(=O)c1cccc(c1)C(=O)NC[C@@H](c1ccccn1)O2] | 1.07 | 501.147 | 502.4 | A |
| 233 | 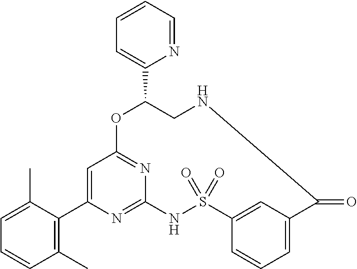 [SMILES: Cc1cccc(C)c1-c1cc2nc(n1)NS(=O)(=O)c1cccc(c1)C(=O)NC[C@@H](c1ccccn1)O2] | 1.07 | 501.147 | 502.4 | A |
| 234 | 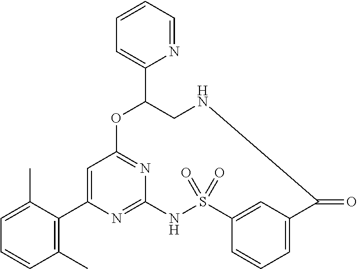 [SMILES: Cc1cccc(C)c1-c1cc2nc(n1)NS(=O)(=O)c1cccc(c1)C(=O)NCC(c1ccccn1)O2] | 1.07 | 501.147 | 502.4 | A |
| 235 | 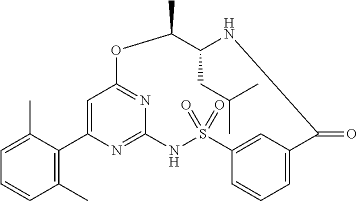 [SMILES: Cc1cccc(C)c1-c1cc2nc(n1)NS(=O)(=O)c1cccc(c1)C(=O)N[C@H](CC(C)C)[C@H](C)O2] | 1.48 | 494.199 | 495.2 | A |
| 236 | 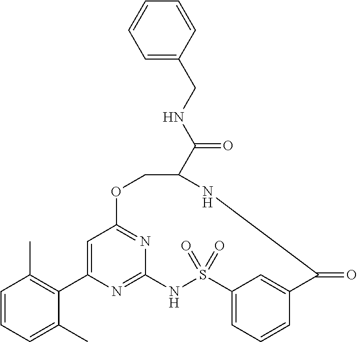 [SMILES: Cc1cccc(C)c1-c1cc2nc(n1)NS(=O)(=O)c1cccc(c1)C(=O)NC(C(=O)NCc1ccccc1)CO2] | 2.05 | 557.173 | 558.4 | A |
| 237 | 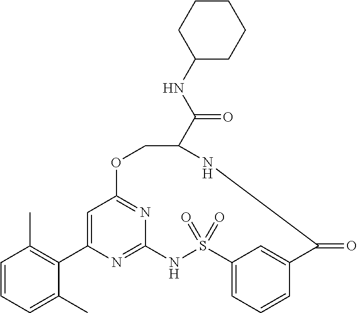 [SMILES: Cc1cccc(C)c1-c1cc2nc(n1)NS(=O)(=O)c1cccc(c1)C(=O)NC(C(=O)NC1CCCCC1)CO2] | 2.12 | 549.205 | 550.4 | A |
| 238 | 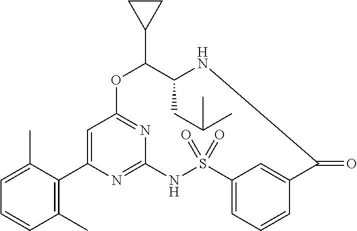 [SMILES: Cc1cccc(C)c1-c1cc2nc(n1)NS(=O)(=O)c1cccc(c1)C(=O)N[C@H](CC(C)C)C(C1CC1)O2] | 1.68 | 520.214 | 521.4 | A |
| 239 | 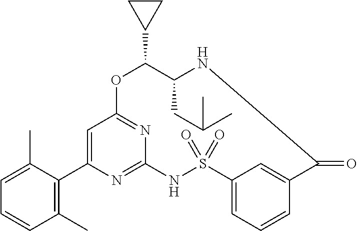 [SMILES: Cc1cccc(C)c1-c1cc2nc(n1)NS(=O)(=O)c1cccc(c1)C(=O)N[C@H](CC(C)C)[C@@H](C1CC1)O2] | 1.64 | 520.214 | 521.5 | A |
| 1 | 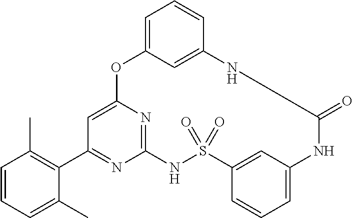 [SMILES: Cc1cccc(C)c1-c1cc2nc(n1)NS(=O)(=O)c1cccc(c1)NC(=O)Nc1cccc(c1)O2] | 1.36 | 487.131 | 488.2 | A |
| 240 | 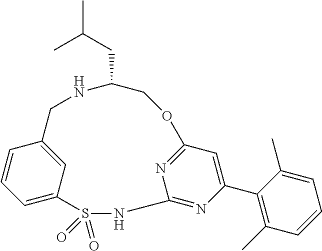 [SMILES: Cc1cccc(C)c1-c1cc2nc(n1)NS(=O)(=O)c1cccc(c1)CN[C@H](CC(C)C)CO2] | 1.89 | 466.204 | 467.2 | U |
| 241 | 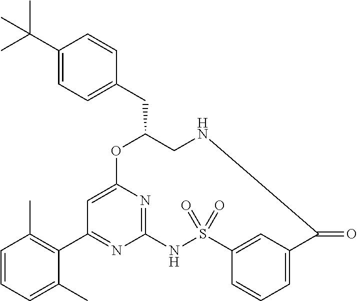 [SMILES: Cc1cccc(C)c1-c1cc2nc(n1)NS(=O)(=O)c1cccc(c1)C(=O)NC[C@@H](Cc1ccc(C(C)(C)C)cc1)O2] | 1.9 | 570.23 | 571.3 | A |
| 242 | 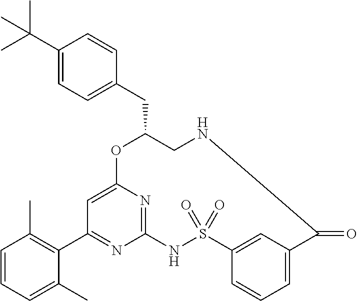 [SMILES: Cc1cccc(C)c1-c1cc2nc(n1)NS(=O)(=O)c1cccc(c1)C(=O)NC[C@@H](Cc1ccc(C(C)(C)C)cc1)O2] | 1.9 | 570.23 | 571.3 | A |
| 243 | 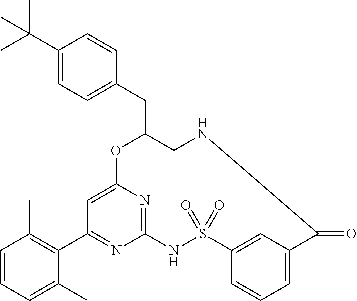 [SMILES: Cc1cccc(C)c1-c1cc2nc(n1)NS(=O)(=O)c1cccc(c1)C(=O)NCC(Cc1ccc(C(C)(C)C)cc1)O2] | 1.9 | 570.23 | 571.38 | A |
| 244 | 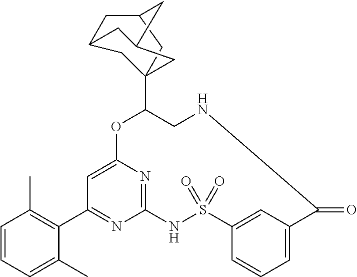 [SMILES: Cc1cccc(C)c1-c1cc2nc(n1)NS(=O)(=O)c1cccc(c1)C(=O)NCC(C13CC4CC(CC(C4)C1)C3)O2] | 1.89 | 558.23 | 559.38 | A |
| 245 | 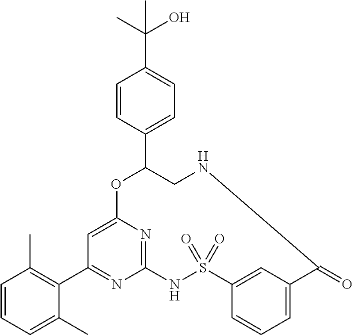 [SMILES: Cc1cccc(C)c1-c1cc2nc(n1)NS(=O)(=O)c1cccc(c1)C(=O)NCC(c1ccc(C(C)(C)O)cc1)O2] | 1.38 | 558.194 | 559.35 | A |
| 246 | 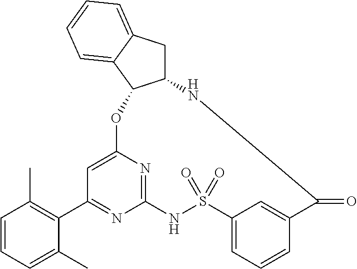 [SMILES: Cc1cccc(C)c1-c1cc2nc(n1)NS(=O)(=O)c1cccc(c1)C(=O)N[C@H]1Cc3ccccc3[C@H]1O2] | 1.48 | 512.152 | 513.29 | A |
| 247 | 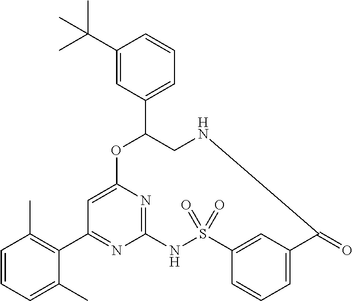 [SMILES: Cc1cccc(C)c1-c1cc2nc(n1)NS(=O)(=O)c1cccc(c1)C(=O)NCC(c1cccc(C(C)(C)C)c1)O2] | 1.86 | 556.214 | 557.37 | A |
| 248 | 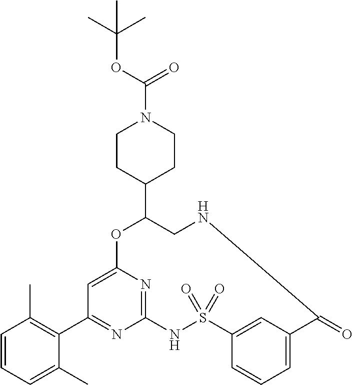 [SMILES: Cc1cccc(C)c1-c1cc2nc(n1)NS(=O)(=O)c1cccc(c1)C(=O)NCC(C1CCN(C(=O)OC(C)(C)C)CC1)O2] | 1.66 | 607.246 | 608.34 | A |
| 249 | 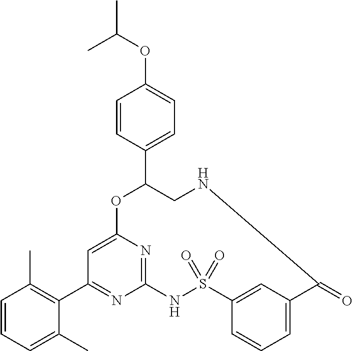 [SMILES: Cc1cccc(C)c1-c1cc2nc(n1)NS(=O)(=O)c1cccc(c1)C(=O)NCC(c1ccc(OC(C)C)cc1)O2] | 1.65 | 558.194 | 559.31 | A |
| 250 | 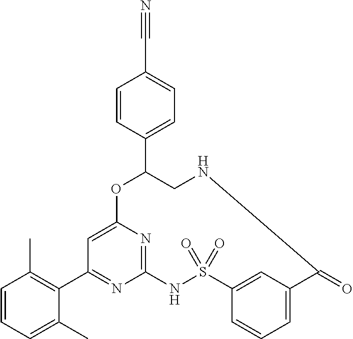 [SMILES: Cc1cccc(C)c1-c1cc2nc(n1)NS(=O)(=O)c1cccc(c1)C(=O)NCC(c1ccc(C#N)cc1)O2] | 1.42 | 525.147 | 526.27 | A |
| 251 | 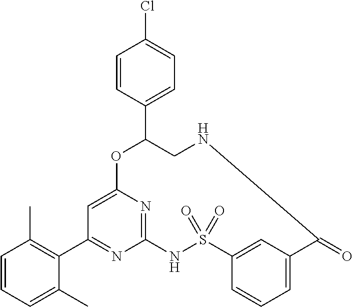 [SMILES: Cc1cccc(C)c1-c1cc2nc(n1)NS(=O)(=O)c1cccc(c1)C(=O)NCC(c1ccc(Cl)cc1)O2] | 1.635 | 534.113 | 535.26 | A |
| 32 | 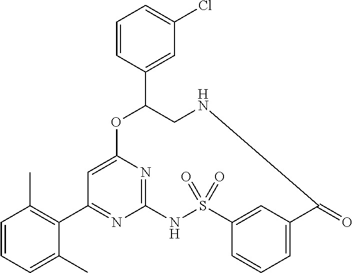 [SMILES: Cc1cccc(C)c1-c1cc2nc(n1)NS(=O)(=O)c1cccc(c1)C(=O)NCC(c1cccc(Cl)c1)O2] | 1.63 | 534.113 | 535.26 | A |
| 252 | 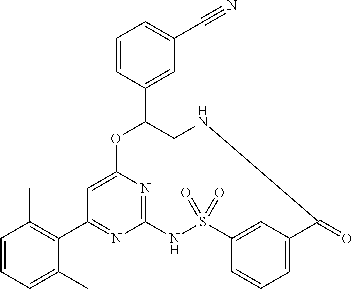 [SMILES: Cc1cccc(C)c1-c1cc2nc(n1)NS(=O)(=O)c1cccc(c1)C(=O)NCC(c1cccc(C#N)c1)O2] | 1.42 | 525.147 | 526.27 | A |
| 31 | 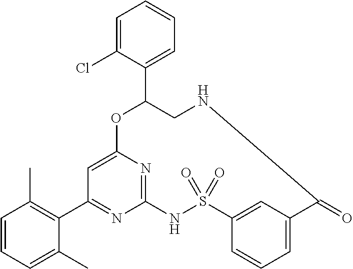 [SMILES: Cc1cccc(C)c1-c1cc2nc(n1)NS(=O)(=O)c1cccc(c1)C(=O)NCC(c1ccccc1Cl)O2] | 1.52 | 534.113 | 535.26 | A |
| 253 | 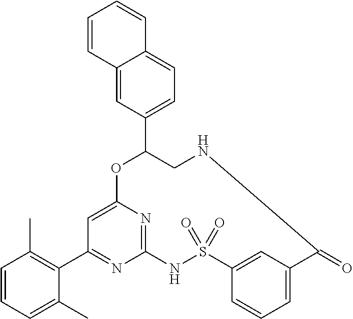 [SMILES: Cc1cccc(C)c1-c1cc2nc(n1)NS(=O)(=O)c1cccc(c1)C(=O)NCC(c1ccc3ccccc3c1)O2] | 1.72 | 550.167 | 551.32 | A |
| 20 | 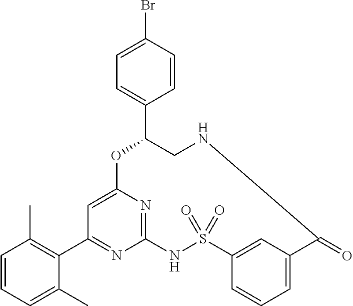 [SMILES: Cc1cccc(C)c1-c1cc2nc(n1)NS(=O)(=O)c1cccc(c1)C(=O)NC[C@@H](c1ccc(Br)cc1)O2] | 1.67 | 578.062 | 581.17 | A |
| 19 | 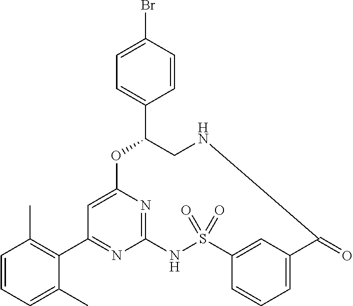 [SMILES: Cc1cccc(C)c1-c1cc2nc(n1)NS(=O)(=O)c1cccc(c1)C(=O)NC[C@@H](c1ccc(Br)cc1)O2] | 1.67 | 578.062 | 581.17 | A |
| 18 | 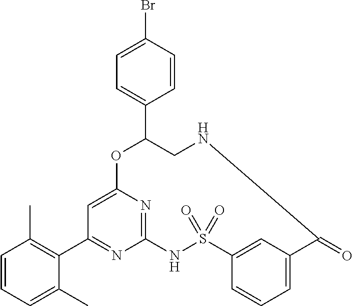 [SMILES: Cc1cccc(C)c1-c1cc2nc(n1)NS(=O)(=O)c1cccc(c1)C(=O)NCC(c1ccc(Br)cc1)O2] | 1.63 | 578.062 | 580.8 | A |
| 254 | 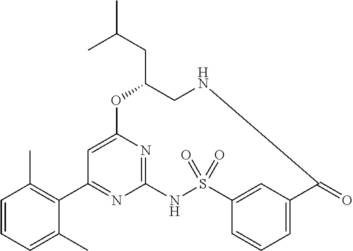 [SMILES: Cc1cccc(C)c1-c1cc2nc(n1)NS(=O)(=O)c1cccc(c1)C(=O)NC[C@@H](CC(C)C)O2] | 1.54 | 480.183 | 481.28 | A |
| 255 | 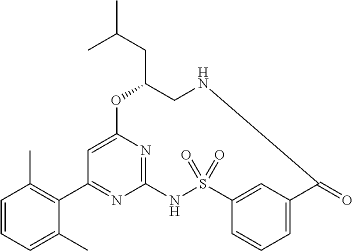 [SMILES: Cc1cccc(C)c1-c1cc2nc(n1)NS(=O)(=O)c1cccc(c1)C(=O)NC[C@@H](CC(C)C)O2] | 1.54 | 480.183 | 481.31 | A |
| 256 | 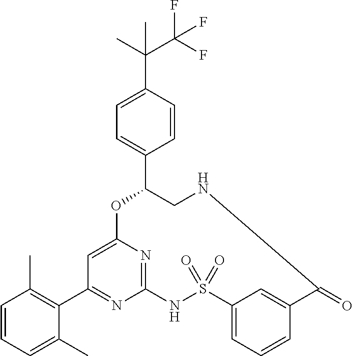 [SMILES: Cc1cccc(C)c1-c1cc2nc(n1)NS(=O)(=O)c1cccc(c1)C(=O)NC[C@@H](c1ccc(C(C)(C)C(F)(F)F)cc1)O2] | 1.83 | 610.186 | 611.31 | A |
| 257 | 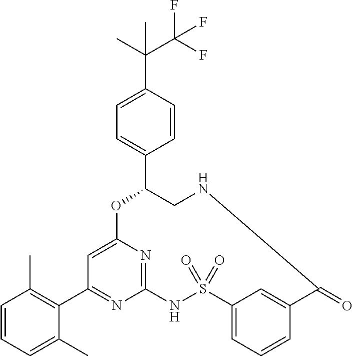 [SMILES: Cc1cccc(C)c1-c1cc2nc(n1)NS(=O)(=O)c1cccc(c1)C(=O)NC[C@@H](c1ccc(C(C)(C)C(F)(F)F)cc1)O2] | 1.83 | 610.186 | 611.31 | A |
| 258 | 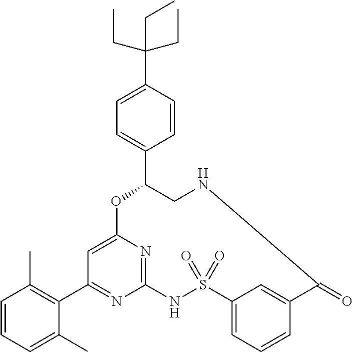 [SMILES: CCC(CC)(CC)c1ccc([C@@H]2CNC(=O)c3cccc(c3)S(=O)(=O)Nc3nc(cc(-c4c(C)cccc4C)n3)O2)cc1] | 2.15 | 598.261 | 599.39 | A |
| 259 | 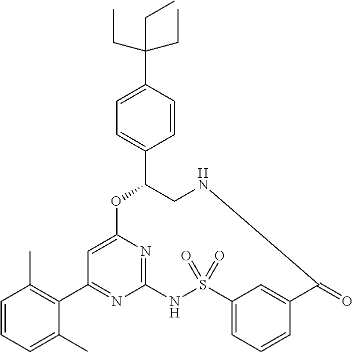 [SMILES: CCC(CC)(CC)c1ccc([C@@H]2CNC(=O)c3cccc(c3)S(=O)(=O)Nc3nc(cc(-c4c(C)cccc4C)n3)O2)cc1] | 2.15 | 598.261 | 599.39 | A |
| 260 | 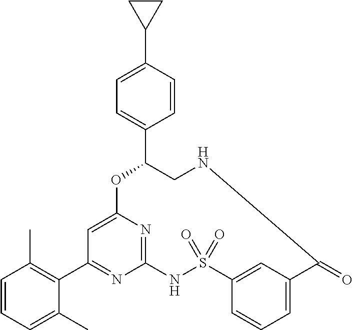 [SMILES: Cc1cccc(C)c1-c1cc2nc(n1)NS(=O)(=O)c1cccc(c1)C(=O)NC[C@@H](c1ccc(C3CC3)cc1)O2] | 2.15 | 540.183 | 541.2 | A |
| 261 | 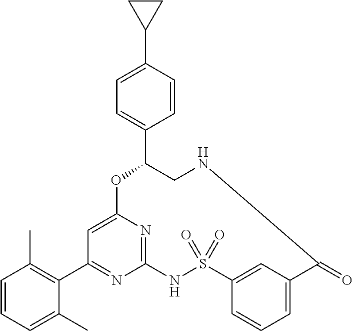 [SMILES: Cc1cccc(C)c1-c1cc2nc(n1)NS(=O)(=O)c1cccc(c1)C(=O)NC[C@@H](c1ccc(C3CC3)cc1)O2] | 2.15 | 540.183 | 541.23 | A |
| 262 | 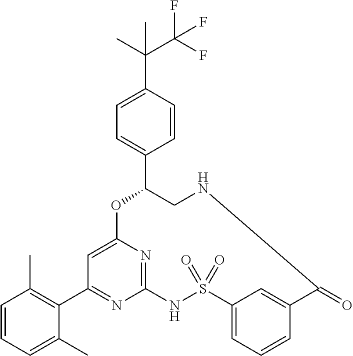 [SMILES: Cc1cccc(C)c1-c1cc2nc(n1)NS(=O)(=O)c1cccc(c1)C(=O)NC[C@@H](c1ccc(C(C)(C)C(F)(F)F)cc1)O2] | 1.84 | 610.186 | 611.27 | A |
| 263 | 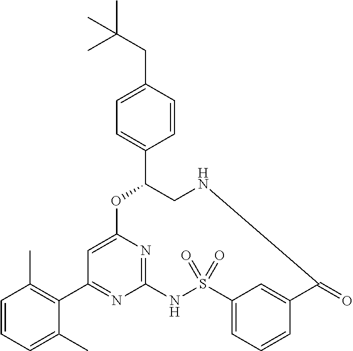 [SMILES: Cc1cccc(C)c1-c1cc2nc(n1)NS(=O)(=O)c1cccc(c1)C(=O)NC[C@@H](c1ccc(CC(C)(C)C)cc1)O2] | 2 | 570.23 | 571.41 | A |
| 264 | 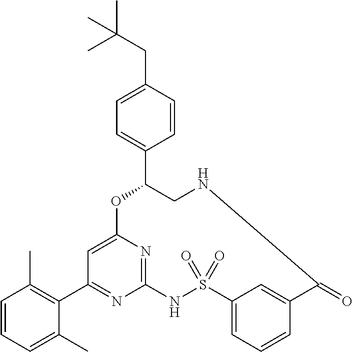 [SMILES: Cc1cccc(C)c1-c1cc2nc(n1)NS(=O)(=O)c1cccc(c1)C(=O)NC[C@@H](c1ccc(CC(C)(C)C)cc1)O2] | 2 | 570.23 | 571.34 | A |
| 265 | 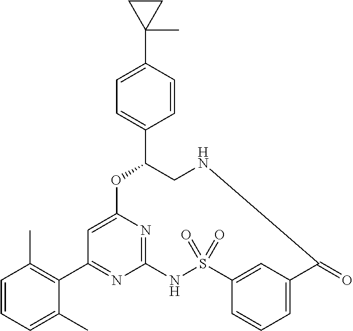 [SMILES: Cc1cccc(C)c1-c1cc2nc(n1)NS(=O)(=O)c1cccc(c1)C(=O)NC[C@@H](c1ccc(C3(C)CC3)cc1)O2] | 1.85 | 554.199 | 555.35 | A |
| 266 | 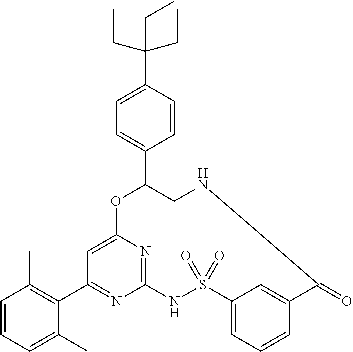 [SMILES: CCC(CC)(CC)c1ccc(C2CNC(=O)c3cccc(c3)S(=O)(=O)Nc3nc(cc(-c4c(C)cccc4C)n3)O2)cc1] | 2.15 | 598.261 | 599.39 | A |
| 267 | 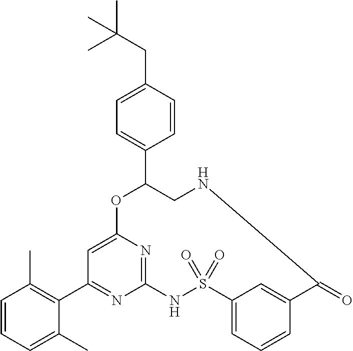 [SMILES: Cc1cccc(C)c1-c1cc2nc(n1)NS(=O)(=O)c1cccc(c1)C(=O)NCC(c1ccc(CC(C)(C)C)cc1)O2] | 2 | 570.23 | 571.31 | A |
| 268 | 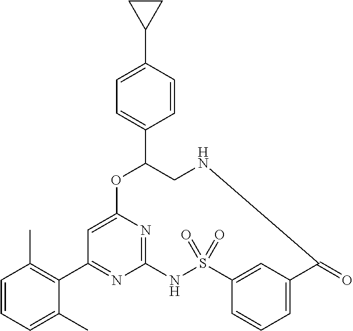 [SMILES: Cc1cccc(C)c1-c1cc2nc(n1)NS(=O)(=O)c1cccc(c1)C(=O)NCC(c1ccc(C3CC3)cc1)O2] | 1.67 | 540.183 | 541 | A |
| 269 | 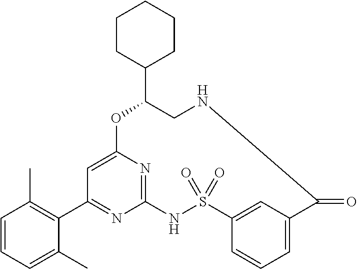 [SMILES: Cc1cccc(C)c1-c1cc2nc(n1)NS(=O)(=O)c1cccc(c1)C(=O)NC[C@@H](C1CCCCC1)O2] | 1.67 | 506.199 | 507.1 | A |
| 270 | 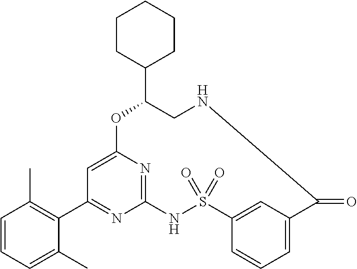 [SMILES: Cc1cccc(C)c1-c1cc2nc(n1)NS(=O)(=O)c1cccc(c1)C(=O)NC[C@@H](C1CCCCC1)O2] | 1.67 | 506.199 | 507.1 | A |
| 271 | 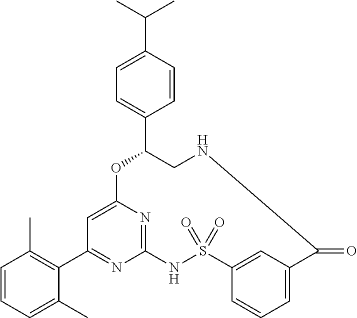 [SMILES: Cc1cccc(C)c1-c1cc2nc(n1)NS(=O)(=O)c1cccc(c1)C(=O)NC[C@@H](c1ccc(C(C)C)cc1)O2] | 1.77 | 542.199 | 543 | A |
| 272 | 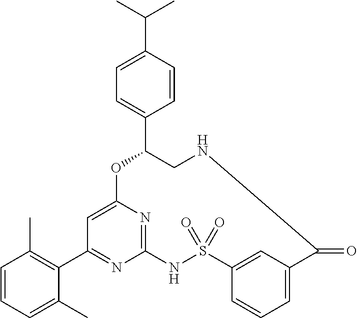 [SMILES: Cc1cccc(C)c1-c1cc2nc(n1)NS(=O)(=O)c1cccc(c1)C(=O)NC[C@@H](c1ccc(C(C)C)cc1)O2] | 1.77 | 542.199 | 543 | A |
| 273 | 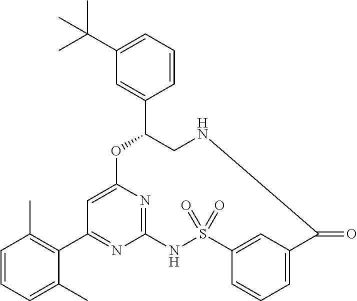 [SMILES: Cc1cccc(C)c1-c1cc2nc(n1)NS(=O)(=O)c1cccc(c1)C(=O)NC[C@@H](c1cccc(C(C)(C)C)c1)O2] | 2.34 | 556.214 | 557.2 | A |
| 274 | 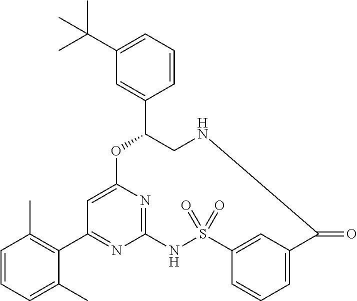 [SMILES: Cc1cccc(C)c1-c1cc2nc(n1)NS(=O)(=O)c1cccc(c1)C(=O)NC[C@@H](c1cccc(C(C)(C)C)c1)O2] | 2.08 | 556.214 | 557.2 | A |
| 275 | 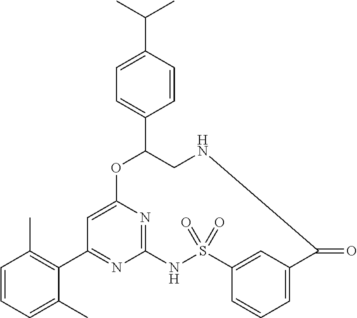 [SMILES: Cc1cccc(C)c1-c1cc2nc(n1)NS(=O)(=O)c1cccc(c1)C(=O)NCC(c1ccc(C(C)C)cc1)O2] | 1.77 | 542.199 | 543 | A |
| 276 | 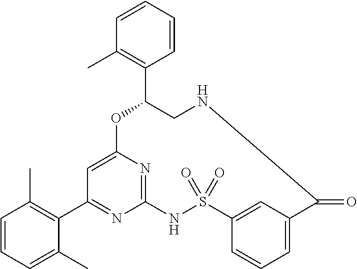 [SMILES: Cc1ccccc1[C@@H]1CNC(=O)c2cccc(c2)S(=O)(=O)Nc2nc(cc(-c3c(C)cccc3C)n2)O1] | 1.54 | 514.167 | 515.31 | A |
| 277 | 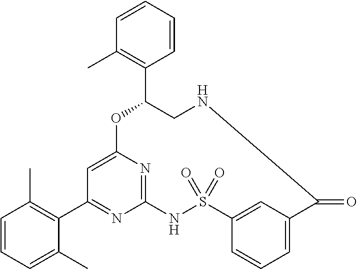 [SMILES: Cc1ccccc1[C@@H]1CNC(=O)c2cccc(c2)S(=O)(=O)Nc2nc(cc(-c3c(C)cccc3C)n2)O1] | 1.54 | 514.167 | 515.27 | A |
| 278 | 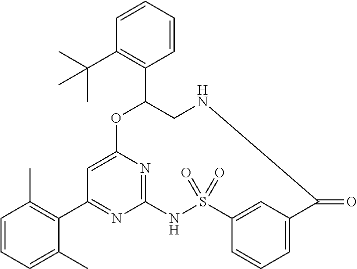 [SMILES: Cc1cccc(C)c1-c1cc2nc(n1)NS(=O)(=O)c1cccc(c1)C(=O)NCC(c1ccccc1C(C)(C)C)O2] | 1.73 | 556.214 | 557.33 | A |
| 280 | 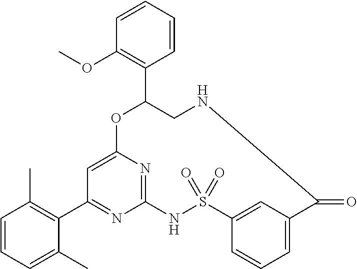 [SMILES: COc1ccccc1C1CNC(=O)c2cccc(c2)S(=O)(=O)Nc2nc(cc(-c3c(C)cccc3C)n2)O1] | 1.5 | 530.162 | 531.29 | A |
| 279 | 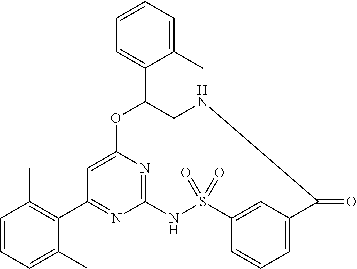 [SMILES: Cc1ccccc1C1CNC(=O)c2cccc(c2)S(=O)(=O)Nc2nc(cc(-c3c(C)cccc3C)n2)O1] | 1.54 | 514.167 | 515.31 | Q |
| 281 | 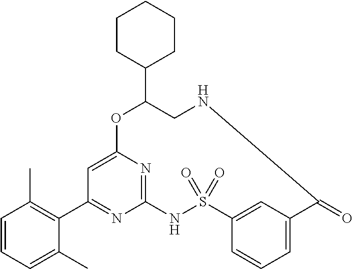 [SMILES: Cc1cccc(C)c1-c1cc2nc(n1)NS(=O)(=O)c1cccc(c1)C(=O)NCC(C1CCCCC1)O2] | 1.71 | 506.199 | 507.35 | A |
| 282 | 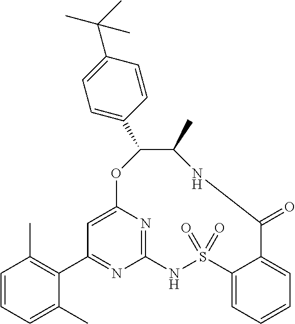 [SMILES: Cc1cccc(C)c1-c1cc2nc(n1)NS(=O)(=O)c1ccccc1C(=O)N[C@H](C)[C@@H](c1ccc(C(C)(C)C)cc1)O2] | 1.78 | 570.23 | 571.3 | A |
| 24 | 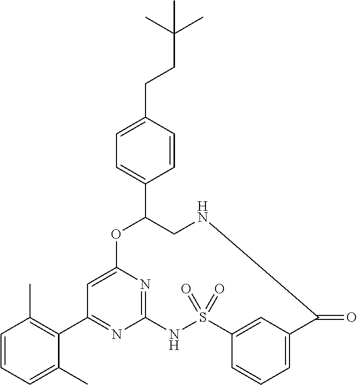 [SMILES: Cc1cccc(C)c1-c1cc2nc(n1)NS(=O)(=O)c1cccc(c1)C(=O)NCC(c1ccc(CCC(C)(C)C)cc1)O2] | 2.08 | 584.246 | 585.2 | A |
| 23 | 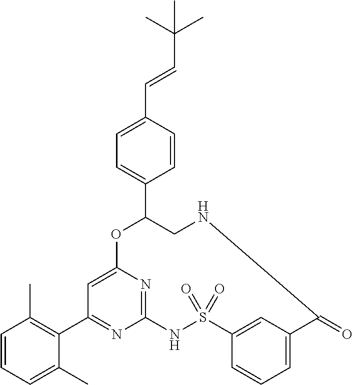 [SMILES: Cc1cccc(C)c1-c1cc2nc(n1)NS(=O)(=O)c1cccc(c1)C(=O)NCC(c1ccc(/C=C/C(C)(C)C)cc1)O2] | 2.02 | 582.23 | 583.3 | A |
| 22 | 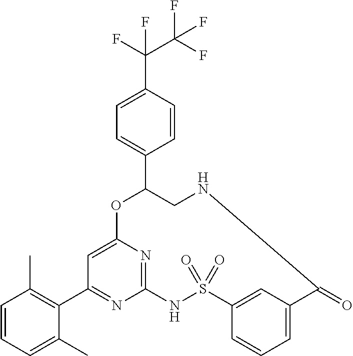 [SMILES: Cc1cccc(C)c1-c1cc2nc(n1)NS(=O)(=O)c1cccc(c1)C(=O)NCC(c1ccc(C(F)(F)C(F)(F)F)cc1)O2] | 1.82 | 618.136 | 619.2 | A |
| 283 | 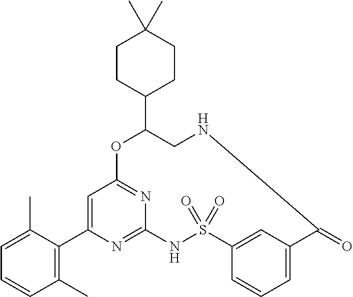 [SMILES: Cc1cccc(C)c1-c1cc2nc(n1)NS(=O)(=O)c1cccc(c1)C(=O)NCC(C1CCC(C)(C)CC1)O2] | 1.81 | 534.23 | 535.5 | A |
| 284 | 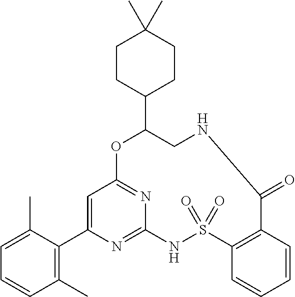 [SMILES: Cc1cccc(C)c1-c1cc2nc(n1)NS(=O)(=O)c1ccccc1C(=O)NCC(C1CCC(C)(C)CC1)O2] | 1.82 | 534.23 | 535.3 | A |
| 285 | 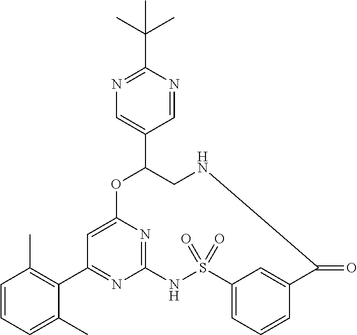 [SMILES: Cc1cccc(C)c1-c1cc2nc(n1)NS(=O)(=O)c1cccc(c1)C(=O)NCC(c1cnc(C(C)(C)C)nc1)O2] | 1.51 | 558.205 | 559.3 | A |
| 286 | 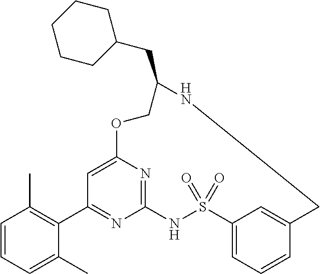 [SMILES: Cc1cccc(C)c1-c1cc2nc(n1)NS(=O)(=O)c1cccc(c1)CN[C@H](CC1CCCCC1)CO2] | 1.28 | 506.235 | 507.5 | A |
| 287 | 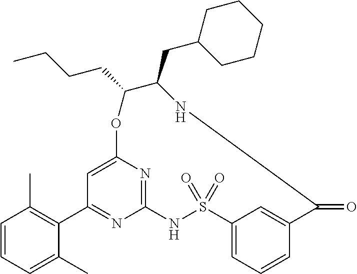 [SMILES: CCCC[C@H]1Oc2cc(-c3c(C)cccc3C)nc(n2)NS(=O)(=O)c2cccc(c2)C(=O)N[C@@H]1CC1CCCCC1] | 1.98 | 576.277 | 577.3 | A |
| 288 | 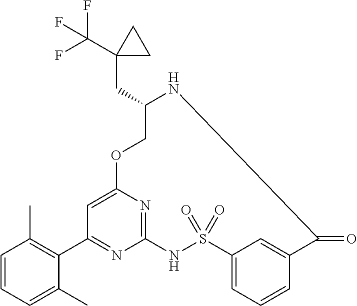 [SMILES: Cc1cccc(C)c1-c1cc2nc(n1)NS(=O)(=O)c1cccc(c1)C(=O)N[C@@H](CC1(C(F)(F)F)CC1)CO2] | 1.48 | 546.155 | 547 | A |
| 289 | 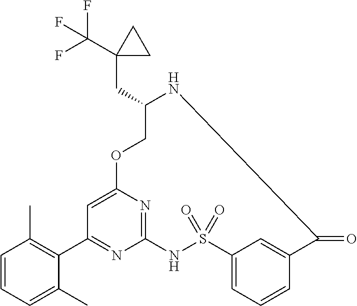 [SMILES: Cc1cccc(C)c1-c1cc2nc(n1)NS(=O)(=O)c1cccc(c1)C(=O)N[C@@H](CC1(C(F)(F)F)CC1)CO2] | 1.48 | 546.155 | 547 | A |

TABLE 4
LCMS characterization data

| | | LCMS | | | |
|---|---|---|---|---|---|
| Compound | | Rt | Calc. | | LCMS |
| number | Structure | (min) | Mass | M + 1 | Method |
| 290 | 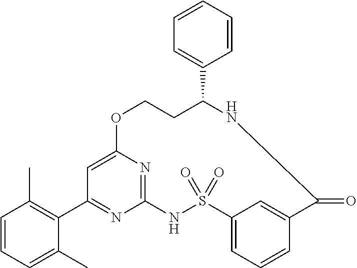 [SMILES: Cc1cccc(C)c1-c1cc2nc(n1)NS(=O)(=O)c1cccc(c1)C(=O)N[C@@H](c1ccccc1)CCO2] | 1.58 | 514.167 | 515 | A |
| 291 | 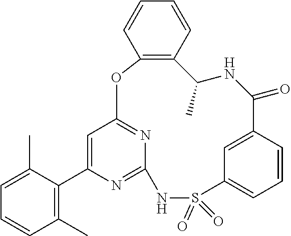 [SMILES: Cc1cccc(C)c1-c1cc2nc(n1)NS(=O)(=O)c1cccc(c1)C(=O)N[C@H](C)c1ccccc1O2] | 1.47 | 500.152 | 501.1 | A |
| 292 | 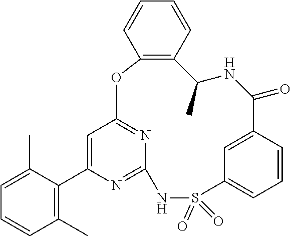 [SMILES: Cc1cccc(C)c1-c1cc2nc(n1)NS(=O)(=O)c1cccc(c1)C(=O)N[C@@H](C)c1ccccc1O2] | 1.47 | 500.152 | 501.1 | A |
| 293 | 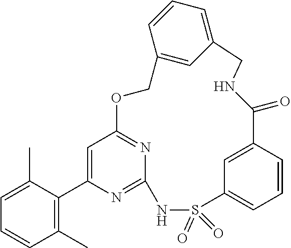 [SMILES: Cc1cccc(C)c1-c1cc2nc(n1)NS(=O)(=O)c1cccc(c1)C(=O)NCc1cccc(c1)CO2] | 1.46 | 500.152 | 501.1 | A |
| 294 | 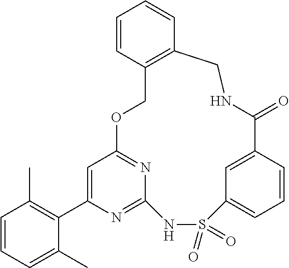 [SMILES: Cc1cccc(C)c1-c1cc2nc(n1)NS(=O)(=O)c1cccc(c1)C(=O)NCc1ccccc1CO2] | 1.48 | 500.152 | 501.1 | A |
| 295 | 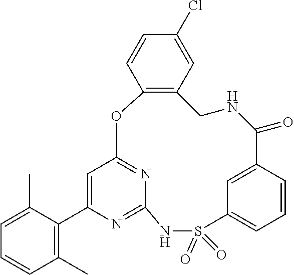 [SMILES: Cc1cccc(C)c1-c1cc2nc(n1)NS(=O)(=O)c1cccc(c1)C(=O)NCc1cc(Cl)ccc1O2] | 1.48 | 520.097 | 521 | A |
| 296 | 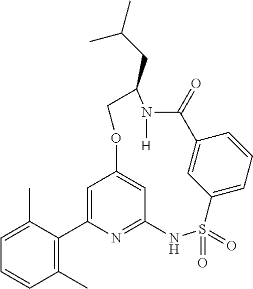 [SMILES: Cc1cccc(C)c1-c1cc2cc(n1)NS(=O)(=O)c1cccc(c1)C(=O)N[C@H](CC(C)C)CO2] | 1.44 | 479.188 | 480.4 | A |
| 297 | 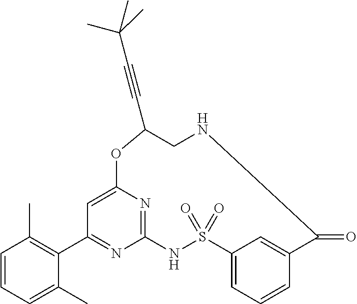 [SMILES: Cc1cccc(C)c1-c1cc2nc(n1)NS(=O)(=O)c1cccc(c1)C(=O)NCC(C#CC(C)(C)C)O2] | 1.63 | 504.183 | 505.3 | A |
| 298 | 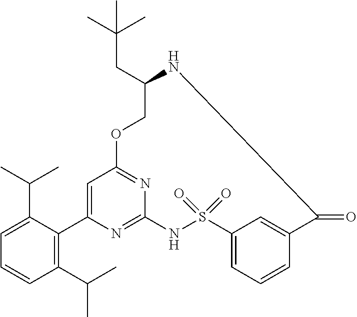 [SMILES: CC(C)c1cccc(C(C)C)c1-c1cc2nc(n1)NS(=O)(=O)c1cccc(c1)C(=O)N[C@H](CC(C)(C)C)CO2] | 1.87 | 550.261 | 551.45 | A |
| 299 | 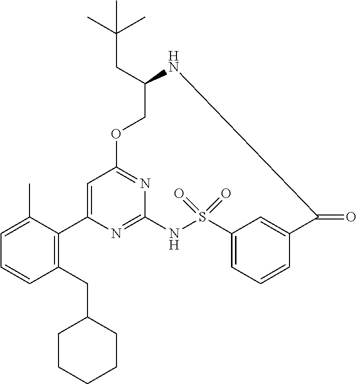 [SMILES: Cc1cccc(CC2CCCCC2)c1-c1cc2nc(n1)NS(=O)(=O)c1cccc(c1)C(=O)N[C@H](CC(C)(C)C)CO2] | 1.98 | 576.277 | 577.28 | A |
| 70 | 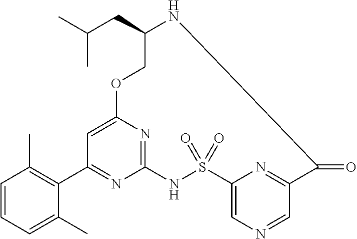 [SMILES: Cc1cccc(C)c1-c1cc2nc(n1)NS(=O)(=O)c1cncc(n1)C(=O)N[C@H](CC(C)C)CO2] | 1.42 | 482.174 | 483.29 | A |
| 300 | 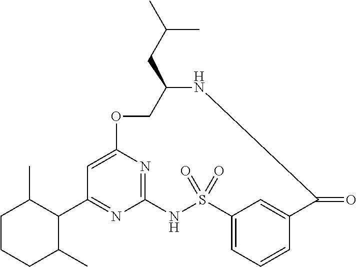 [SMILES: CC(C)C[C@@H]1COc2cc(C3C(C)CCCC3C)nc(n2)NS(=O)(=O)c2cccc(c2)C(=O)N1] | 2.28 | 486.23 | 487.3 | I |
| 301 | 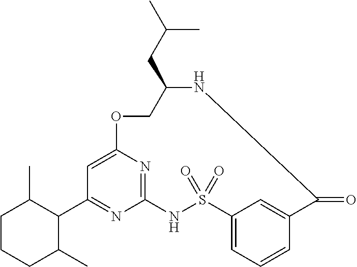 [SMILES: CC(C)C[C@@H]1COc2cc(C3C(C)CCCC3C)nc(n2)NS(=O)(=O)c2cccc(c2)C(=O)N1] | 2.3 | 486.23 | 487.3 | I |
| 302 | 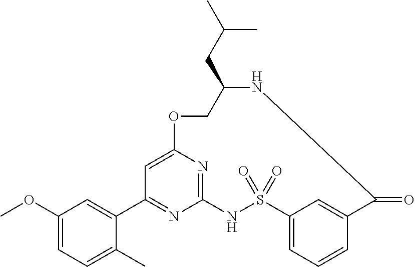 [SMILES: COc1ccc(C)c(-c2cc3nc(n2)NS(=O)(=O)c2cccc(c2)C(=O)N[C@H](CC(C)C)CO3)c1] | 1.5 | 496.178 | 497.3 | A |
| 303 | 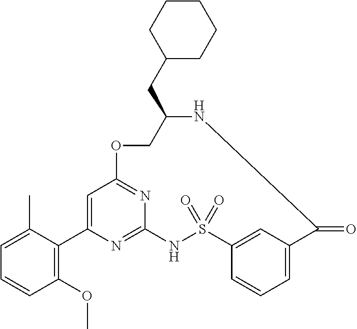 [SMILES: COc1cccc(C)c1-c1cc2nc(n1)NS(=O)(=O)c1cccc(c1)C(=O)N[C@H](CC1CCCCC1)CO2] | 1.66 | 536.209 | 537.3 | A |
| 304 | 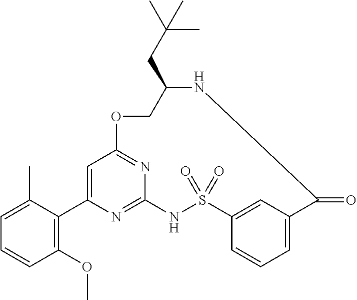 [SMILES: COc1cccc(C)c1-c1cc2nc(n1)NS(=O)(=O)c1cccc(c1)C(=O)N[C@H](CC(C)(C)C)CO2] | 1.5 | 510.194 | 511.3 | A |
| 305 | 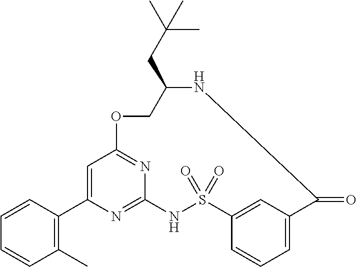 [SMILES: Cc1ccccc1-c1cc2nc(n1)NS(=O)(=O)c1cccc(c1)C(=O)N[C@H](CC(C)(C)C)CO2] | 1.54 | 480.183 | 481.3 | A |
| 306 | 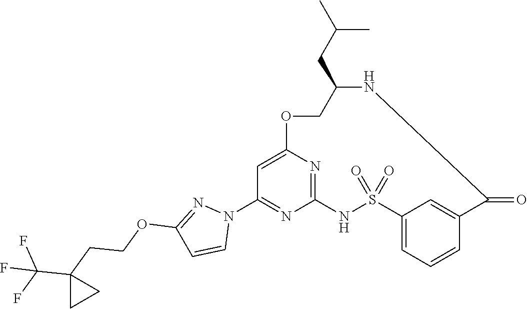 [SMILES: CC(C)C[C@@H]1COc2cc(-n3ccc(OCCC4(C(F)(F)F)CC4)n3)nc(n2)NS(=O)(=O)c2cccc(c2)C(=O)N1] | 2.01 | 594.187 | 595.3 | A |
| 307 | 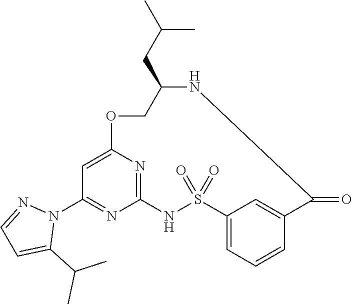 [SMILES: CC(C)C[C@@H]1COc2cc(-n3nccc3C(C)C)nc(n2)NS(=O)(=O)c2cccc(c2)C(=O)N1] | 1.86 | 484.189 | 485.3 | A |
| 308 | 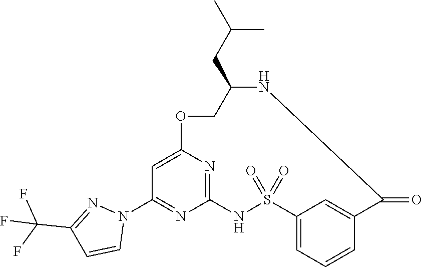 [SMILES: CC(C)C[C@@H]1COc2cc(-n3ccc(C(F)(F)F)n3)nc(n2)NS(=O)(=O)c2cccc(c2)C(=O)N1] | 1.8 | 510.13 | 511.3 | A |
| 309 | 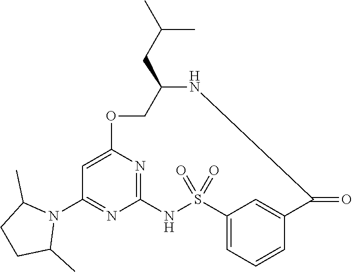 [SMILES: CC(C)C[C@@H]1COc2cc(N3C(C)CCC3C)nc(n2)NS(=O)(=O)c2cccc(c2)C(=O)N1] | 1.67 | 473.21 | 474.3 | A |
| 310 | 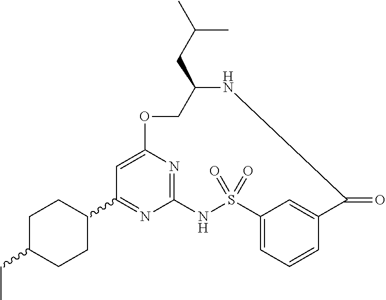 [SMILES: CCC1CCC(c2cc3nc(n2)NS(=O)(=O)c2cccc(c2)C(=O)N[C@H](CC(C)C)CO3)CC1] | 1.8 | 486.23 | 487.5 | A |
| 311 | 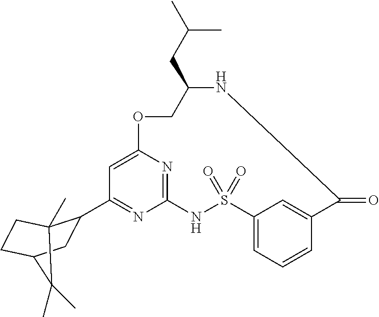 [SMILES: CC(C)C[C@@H]1COc2cc(C3CC4CCC3(C)C4(C)C)nc(n2)NS(=O)(=O)c2cccc(c2)C(=O)N1] | 1.83 | 512.246 | 513.4 | A |
| 312 | 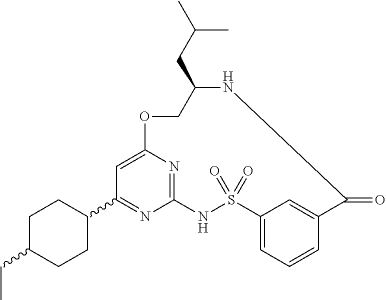 [SMILES: CCC1CCC(c2cc3nc(n2)NS(=O)(=O)c2cccc(c2)C(=O)N[C@H](CC(C)C)CO3)CC1] | 1.76 | 486.23 | 487.4 | A |
| 313 | 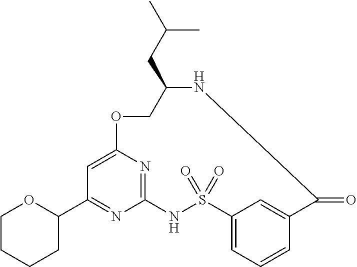 [SMILES: CC(C)C[C@@H]1COc2cc(C3CCCCO3)nc(n2)NS(=O)(=O)c2cccc(c2)C(=O)N1] | 1.36 | 460.178 | 461.3 | A |
| 314 | 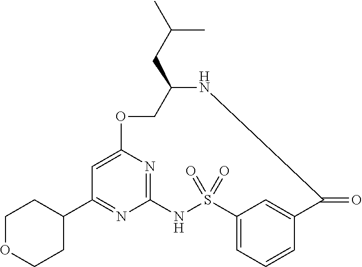 [SMILES: CC(C)C[C@@H]1COc2cc(C3CCOCC3)nc(n2)NS(=O)(=O)c2cccc(c2)C(=O)N1] | 1.1 | 460.178 | 461.3 | A |
| 315 | 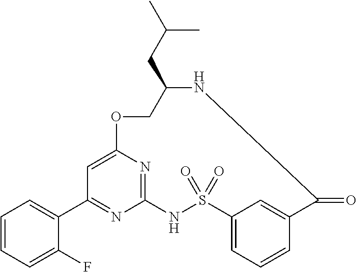 [SMILES: CC(C)C[C@@H]1COc2cc(-c3ccccc3F)nc(n2)NS(=O)(=O)c2cccc(c2)C(=O)N1] | 1.54 | 470.142 | 471.2 | A |
| 316 | 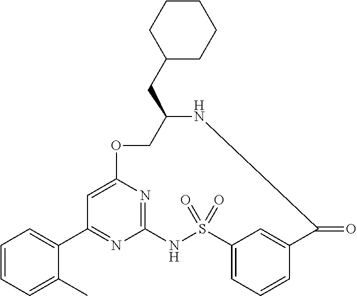 [SMILES: Cc1ccccc1-c1cc2nc(n1)NS(=O)(=O)c1cccc(c1)C(=O)N[C@H](CC1CCCCC1)CO2] | 1.72 | 506.199 | 507.3 | A |
| 317 | 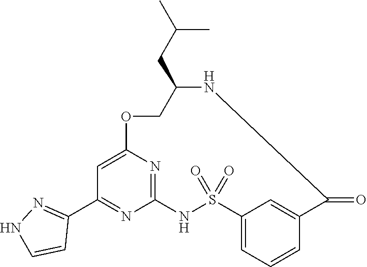 [SMILES: CC(C)C[C@@H]1COc2cc(-c3cc[nH]n3)nc(n2)NS(=O)(=O)c2cccc(c2)C(=O)N1] | 1.06 | 442.142 | 443.2 | A |
| 318 | 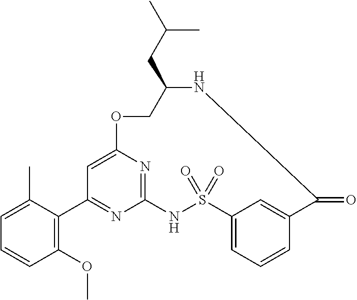 [SMILES: COc1cccc(C)c1-c1cc2nc(n1)NS(=O)(=O)c1cccc(c1)C(=O)N[C@H](CC(C)C)CO2] | 1.46 | 496.178 | 497.3 | A |
| 319 | 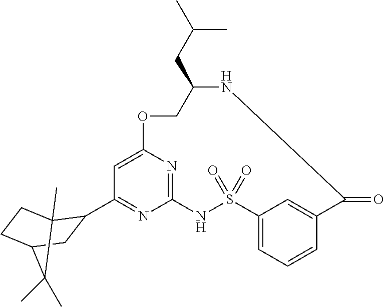 [SMILES: CC(C)C[C@@H]1COc2cc(C3CC4CCC3(C)C4(C)C)nc(n2)NS(=O)(=O)c2cccc(c2)C(=O)N1] | 1.83 | 510.23 | 511.3 | A |
| 320 | 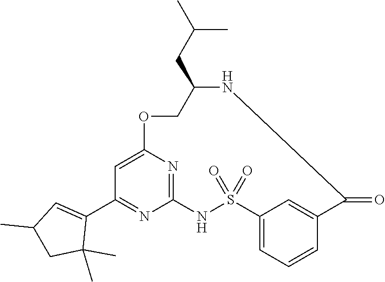 [SMILES: CC(C)C[C@@H]1COc2cc(C3=CC(C)CC3(C)C)nc(n2)NS(=O)(=O)c2cccc(c2)C(=O)N1] | 1.83 | 484.214 | 485.3 | A |
| 321 | 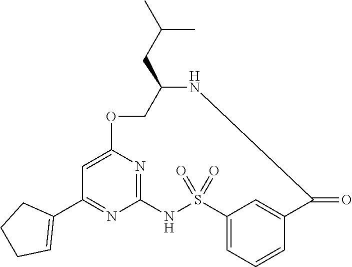 [SMILES: CC(C)C[C@@H]1COc2cc(C3=CCCC3)nc(n2)NS(=O)(=O)c2cccc(c2)C(=O)N1] | 1.46 | 442.167 | 443.3 | A |
| 322 | 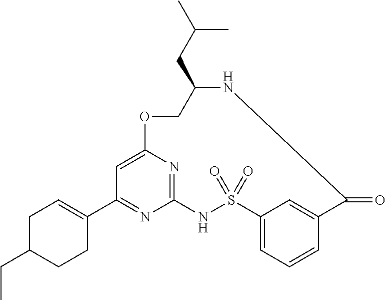 [SMILES: CCC1CC=C(c2cc3nc(n2)NS(=O)(=O)c2cccc(c2)C(=O)N[C@H](CC(C)C)CO3)CC1] | 1.81 | 484.214 | 485.2 | A |
| 323 | 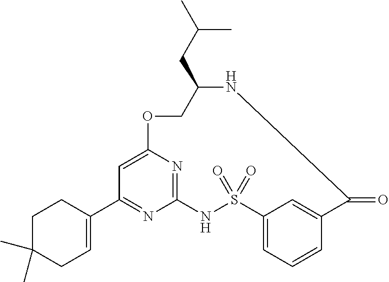 [SMILES: CC(C)C[C@@H]1COc2cc(C3=CCC(C)(C)CC3)nc(n2)NS(=O)(=O)c2cccc(c2)C(=O)N1] | 1.76 | 484.214 | 485.2 | A |
| 324 | 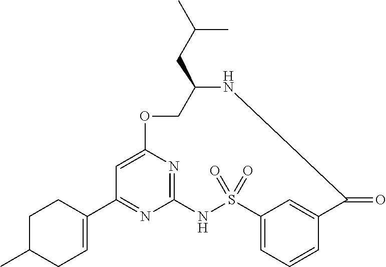 [SMILES: CC(C)C[C@@H]1COc2cc(C3=CCC(C)CC3)nc(n2)NS(=O)(=O)c2cccc(c2)C(=O)N1] | 1.67 | 470.199 | 471.1 | A |
| 325 | 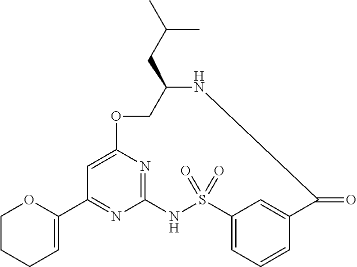 [SMILES: CC(C)C[C@@H]1COc2cc(C3=CCCCO3)nc(n2)NS(=O)(=O)c2cccc(c2)C(=O)N1] | 1.41 | 458.162 | 459.1 | A |
| 326 | 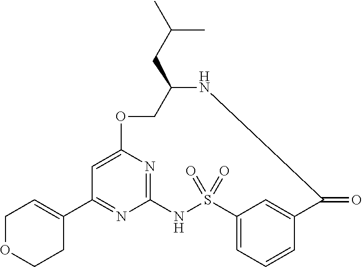 [SMILES: CC(C)C[C@@H]1COc2cc(C3=CCOCC3)nc(n2)NS(=O)(=O)c2cccc(c2)C(=O)N1] | 1.17 | 458.162 | 459.1 | A |
| 327 | 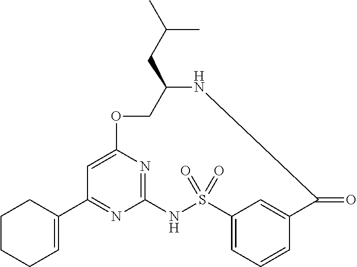 [SMILES: CC(C)C[C@@H]1COc2cc(C3=CCCCC3)nc(n2)NS(=O)(=O)c2cccc(c2)C(=O)N1] | 1.53 | 456.183 | 457.1 | A |
| 328 | 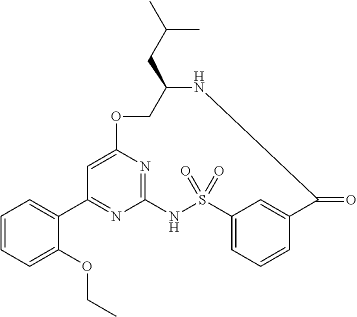 [SMILES: CCOc1ccccc1-c1cc2nc(n1)NS(=O)(=O)c1cccc(c1)C(=O)N[C@H](CC(C)C)CO2] | 1.54 | 496.178 | 497.3 | A |
| 329 | 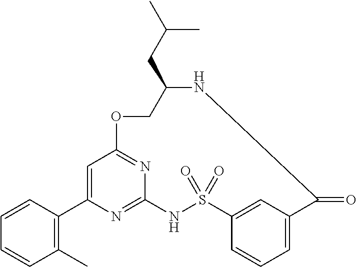 [SMILES: Cc1ccccc1-c1cc2nc(n1)NS(=O)(=O)c1cccc(c1)C(=O)N[C@H](CC(C)C)CO2] | 1.49 | 466.167 | 467.3 | A |
| 330 | 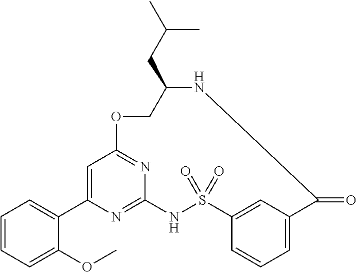 [SMILES: COc1ccccc1-c1cc2nc(n1)NS(=O)(=O)c1cccc(c1)C(=O)N[C@H](CC(C)C)CO2] | 1.42 | 482.162 | 483.2 | A |
| 331 | 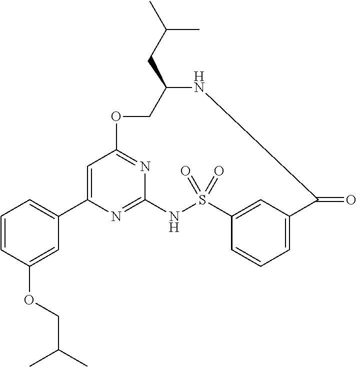 [SMILES: CC(C)COc1cccc(-c2cc3nc(n2)NS(=O)(=O)c2cccc(c2)C(=O)N[C@H](CC(C)C)CO3)c1] | 1.97 | 524.209 | 525.5 | A |
| 332 | 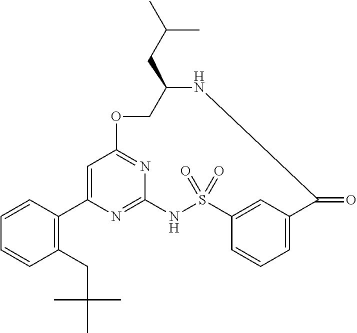 [SMILES: CC(C)C[C@@H]1COc2cc(-c3ccccc3CC(C)(C)C)nc(n2)NS(=O)(=O)c2cccc(c2)C(=O)N1] | 1.82 | 522.23 | 523.3 | A |
| 333 | 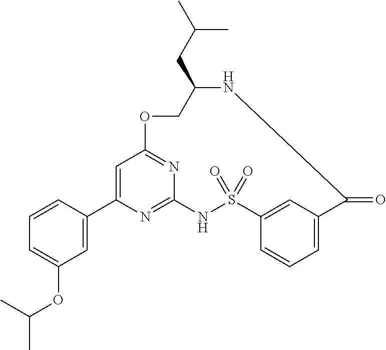 [SMILES: CC(C)C[C@@H]1COc2cc(-c3cccc(OC(C)C)c3)nc(n2)NS(=O)(=O)c2cccc(c2)C(=O)N1] | 1.78 | 510.194 | 511.3 | A |
| 334 | 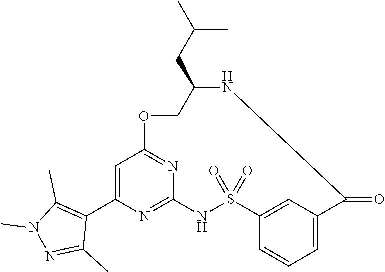 [SMILES: Cc1nn(C)c(C)c1-c1cc2nc(n1)NS(=O)(=O)c1cccc(c1)C(=O)N[C@H](CC(C)C)CO2] | 1.14 | 484.189 | 485.2 | A |
| 335 | 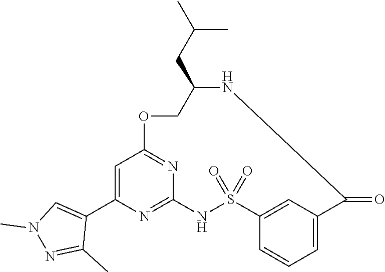 [SMILES: Cc1nn(C)cc1-c1cc2nc(n1)NS(=O)(=O)c1cccc(c1)C(=O)N[C@H](CC(C)C)CO2] | 1.14 | 470.174 | 471.2 | A |
| 336 | 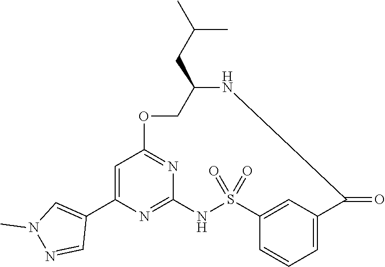 [SMILES: CC(C)C[C@@H]1COc2cc(-c3cnn(C)c3)nc(n2)NS(=O)(=O)c2cccc(c2)C(=O)N1] | 1.08 | 456.158 | 457.1 | A |
| 337 | 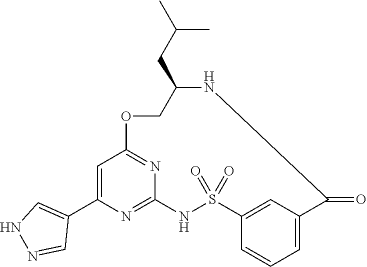 [SMILES: CC(C)C[C@@H]1COc2cc(-c3cn[nH]c3)nc(n2)NS(=O)(=O)c2cccc(c2)C(=O)N1] | 0.98 | 442.142 | 443.2 | A |
| 338 | 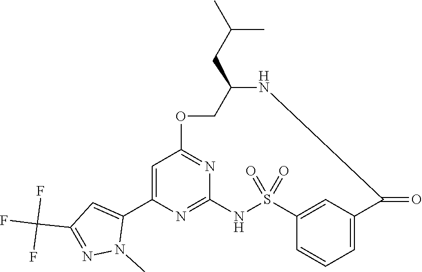 [SMILES: CC(C)C[C@@H]1COc2cc(-c3cc(C(F)(F)F)nn3C)nc(n2)NS(=O)(=O)c2cccc(c2)C(=O)N1] | 1.69 | 524.145 | 525.1 | A |
| 339 | 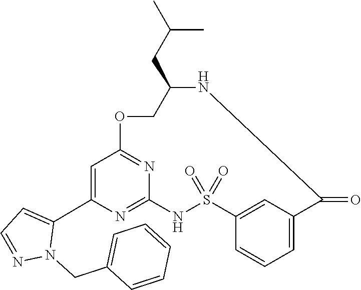 [SMILES: CC(C)C[C@@H]1COc2cc(-c3ccnn3Cc3ccccc3)nc(n2)NS(=O)(=O)c2cccc(c2)C(=O)N1] | 1.61 | 532.189 | 533.3 | A |
| 340 | 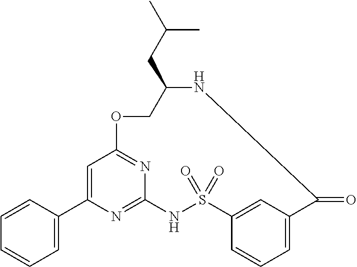 [SMILES: CC(C)C[C@@H]1COc2cc(-c3ccccc3)nc(n2)NS(=O)(=O)c2cccc(c2)C(=O)N1] | 0.15 | 452.152 | 453.2 | A |
| 341 | 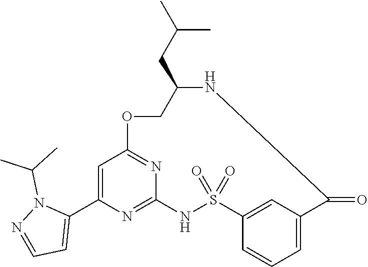 [SMILES: CC(C)C[C@@H]1COc2cc(-c3ccnn3C(C)C)nc(n2)NS(=O)(=O)c2cccc(c2)C(=O)N1] | 1.53 | 484.189 | 485.3 | A |
| 342 | 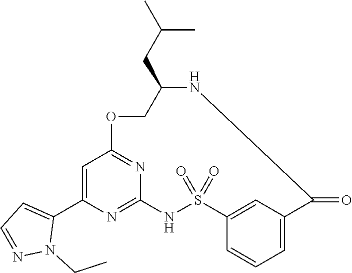 [SMILES: CCn1nccc1-c1cc2nc(n1)NS(=O)(=O)c1cccc(c1)C(=O)N[C@H](CC(C)C)CO2] | 1.38 | 470.174 | 471.1 | A |
| 343 | 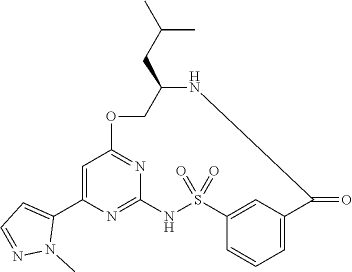 [SMILES: CC(C)C[C@@H]1COc2cc(-c3ccnn3C)nc(n2)NS(=O)(=O)c2cccc(c2)C(=O)N1] | 1.27 | 456.158 | 457.1 | A |
| 344 | 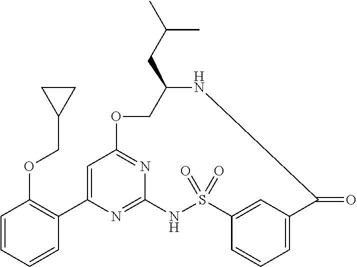 [SMILES: CC(C)C[C@@H]1COc2cc(-c3ccccc3OCC3CC3)nc(n2)NS(=O)(=O)c2cccc(c2)C(=O)N1] | 1.62 | 522.194 | 523.1 | A |
| 345 | 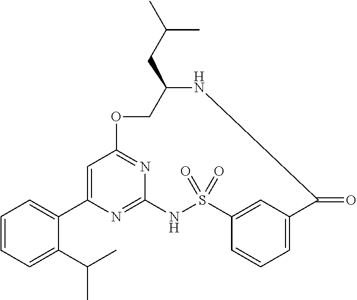 [SMILES: CC(C)C[C@@H]1COc2cc(-c3ccccc3C(C)C)nc(n2)NS(=O)(=O)c2cccc(c2)C(=O)N1] | 1.63 | 494.199 | 495.1 | A |
| 346 | 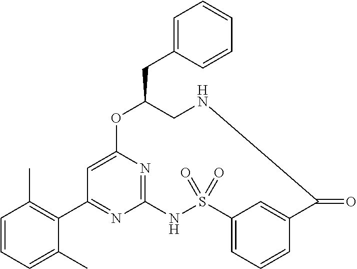 [SMILES: Cc1cccc(C)c1-c1cc2nc(n1)NS(=O)(=O)c1cccc(c1)C(=O)NC[C@H](Cc1ccccc1)O2] | 1.51 | 514.167 | 515.1 | A |
| 347 | 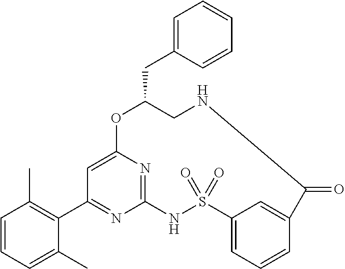 [SMILES: Cc1cccc(C)c1-c1cc2nc(n1)NS(=O)(=O)c1cccc(c1)C(=O)NC[C@@H](Cc1ccccc1)O2] | 1.51 | 514.167 | 515 | A |
| 348 | 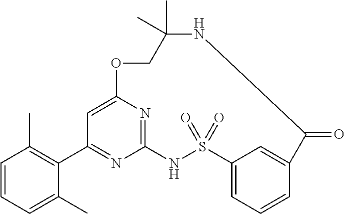 [SMILES: Cc1cccc(C)c1-c1cc2nc(n1)NS(=O)(=O)c1cccc(c1)C(=O)NC(C)(C)CO2] | 1.27 | 452.152 | 453.1 | A |
| 349 | 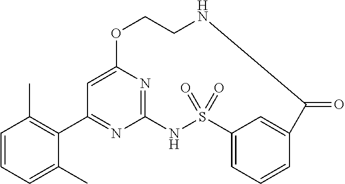 [SMILES: Cc1cccc(C)c1-c1cc2nc(n1)NS(=O)(=O)c1cccc(c1)C(=O)NCCO2] | 1.11 | 424.121 | 425.2 | A |
| 350 | 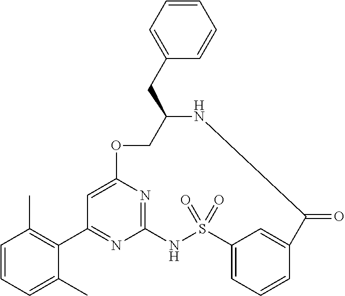 [SMILES: Cc1cccc(C)c1-c1cc2nc(n1)NS(=O)(=O)c1cccc(c1)C(=O)N[C@H](Cc1ccccc1)CO2] | 1.52 | 514.167 | 515.2 | A |
| 351 | 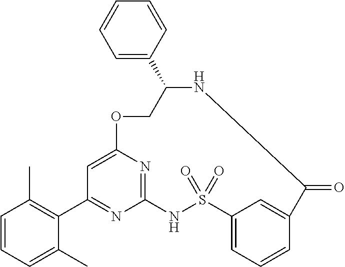 [SMILES: Cc1cccc(C)c1-c1cc2nc(n1)NS(=O)(=O)c1cccc(c1)C(=O)N[C@@H](c1ccccc1)CO2] | 1.47 | 500.152 | 501.3 | A |
| 352 | 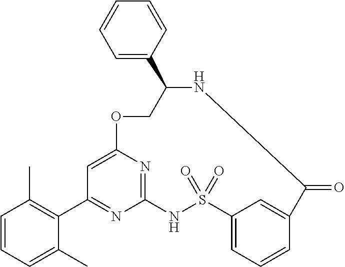 [SMILES: Cc1cccc(C)c1-c1cc2nc(n1)NS(=O)(=O)c1cccc(c1)C(=O)N[C@H](c1ccccc1)CO2] | 1.47 | 500.152 | 501.2 | A |
| 353 | 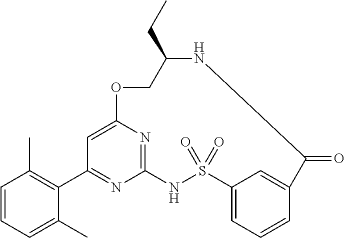 [SMILES: CC[C@@H]1COc2cc(-c3c(C)cccc3C)nc(n2)NS(=O)(=O)c2cccc(c2)C(=O)N1] | 1.32 | 452.152 | 453.2 | A |
| 354 | 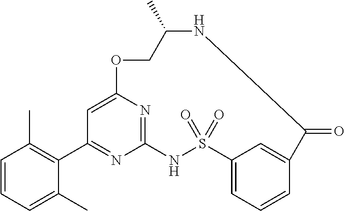 [SMILES: Cc1cccc(C)c1-c1cc2nc(n1)NS(=O)(=O)c1cccc(c1)C(=O)N[C@@H](C)CO2] | 1.22 | 438.136 | 439.2 | A |
| 355 | 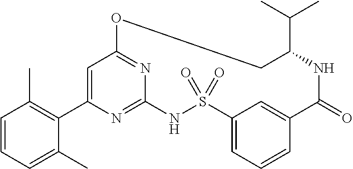 [SMILES: Cc1cccc(C)c1-c1cc2nc(n1)NS(=O)(=O)c1cccc(c1)C(=O)N[C@@H](C(C)C)CO2] | 1.54 | 466.167 | 467.2 | A |
| 356 | 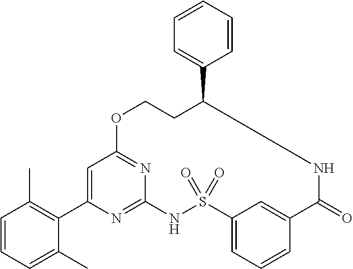 [SMILES: Cc1cccc(C)c1-c1cc2nc(n1)NS(=O)(=O)c1cccc(c1)C(=O)N[C@H](c1ccccc1)CCO2] | 1.56 | 514.167 | 515.3 | A |
| 357 | 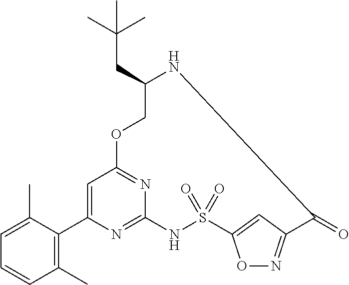 [SMILES: Cc1cccc(C)c1-c1cc2nc(n1)NS(=O)(=O)c1cc(no1)C(=O)N[C@H](CC(C)(C)C)CO2] | 1.6 | 485.173 | 486 | A |
| 358 | 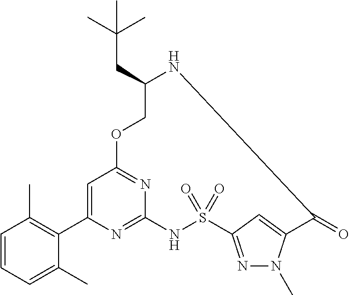 [SMILES: Cc1cccc(C)c1-c1cc2nc(n1)NS(=O)(=O)c1cc(n(C)n1)C(=O)N[C@H](CC(C)(C)C)CO2] | 1.57 | 498.205 | 499.14 | A |
| 77 | 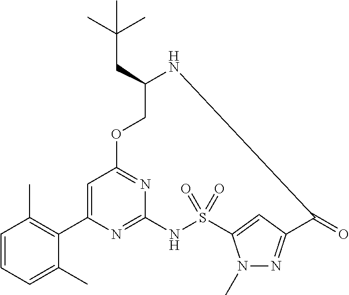 [SMILES: Cc1cccc(C)c1-c1cc2nc(n1)NS(=O)(=O)c1cc(nn1C)C(=O)N[C@H](CC(C)(C)C)CO2] | 1.59 | 498.205 | 499.14 | A |
| 359 | 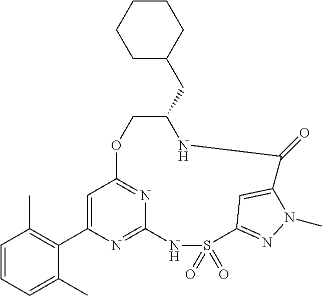 [SMILES: Cc1cccc(C)c1-c1cc2nc(n1)NS(=O)(=O)c1cc(n(C)n1)C(=O)N[C@@H](CC1CCCCC1)CO2] | 1.66 | 524.221 | 525.6 | A |
| 360 | 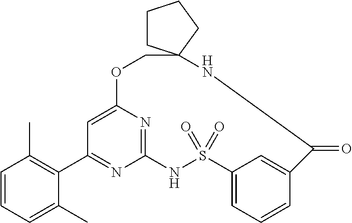 [SMILES: Cc1cccc(C)c1-c1cc2nc(n1)NS(=O)(=O)c1cccc(c1)C(=O)NC1(CCCC1)CO2] | 1.38 | 478.167 | 479.3 | A |
| 361 | 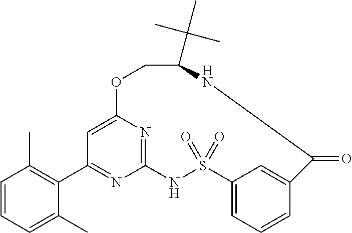 [SMILES: Cc1cccc(C)c1-c1cc2nc(n1)NS(=O)(=O)c1cccc(c1)C(=O)N[C@H](C(C)(C)C)CO2] | 1.47 | 480.183 | 481.4 | A |
| 362 | 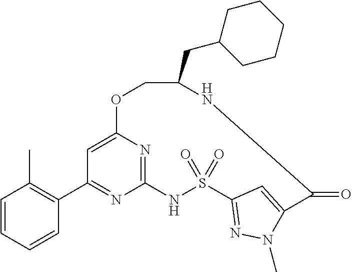 [SMILES: Cc1ccccc1-c1cc2nc(n1)NS(=O)(=O)c1cc(n(C)n1)C(=O)N[C@H](CC1CCCCC1)CO2] | 2.19 | 510.205 | 511.6 | Q |
| 363 | 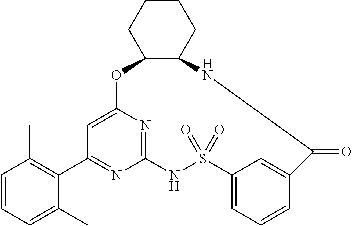 [SMILES: Cc1cccc(C)c1-c1cc2nc(n1)NS(=O)(=O)c1cccc(c1)C(=O)N[C@@H]1CCCC[C@@H]1O2] | 1.54 | 478.167 | 479.3 | A |
| 364 | 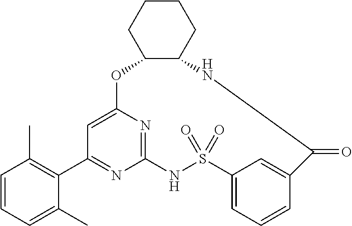 [SMILES: Cc1cccc(C)c1-c1cc2nc(n1)NS(=O)(=O)c1cccc(c1)C(=O)N[C@H]1CCCC[C@H]1O2] | 1.55 | 478.167 | 479.3 | A |
| 365 | 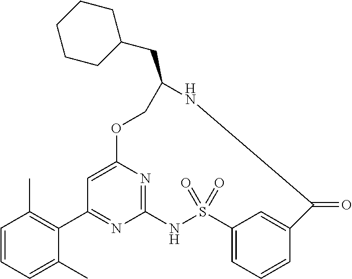 [SMILES: Cc1cccc(C)c1-c1cc2nc(n1)NS(=O)(=O)c1cccc(c1)C(=O)N[C@H](CC1CCCCC1)CO2] | 1.718 | 520.214 | 521.2 | A |
| 366 | 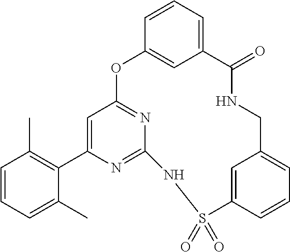 [SMILES: Cc1cccc(C)c1-c1cc2nc(n1)NS(=O)(=O)c1cccc(c1)CNC(=O)c1cccc(c1)O2] | 1.88 | 486.136 | 487.4 | A |
| 367 | 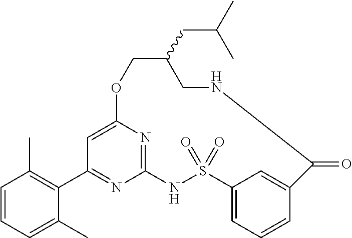 [SMILES: Cc1cccc(C)c1-c1cc2nc(n1)NS(=O)(=O)c1cccc(c1)C(=O)NCC(CC(C)C)CO2] | 1.67 | 494.199 | 495.1 | A |
| 368 | 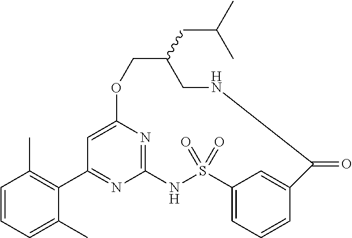 [SMILES: Cc1cccc(C)c1-c1cc2nc(n1)NS(=O)(=O)c1cccc(c1)C(=O)NCC(CC(C)C)CO2] | 1.67 | 494.199 | 495 | A |
| 369 | 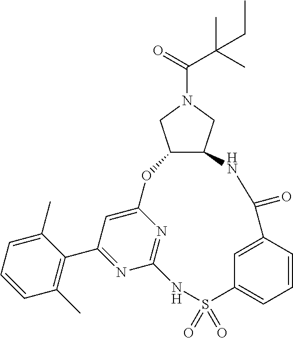 [SMILES: CCC(C)(C)C(=O)N1C[C@H]2NC(=O)c3cccc(c3)S(=O)(=O)Nc3nc(cc(-c4c(C)cccc4C)n3)O[C@@H]2C1] | 1.39 | 563.22 | 564.2 | A |
| 370 | 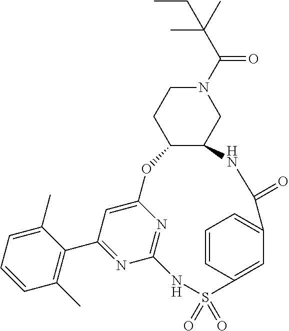 [SMILES: CCC(C)(C)C(=O)N1CC[C@H]2Oc3cc(-c4c(C)cccc4C)nc(n3)NS(=O)(=O)c3cccc(c3)C(=O)N[C@@H]2C1] | 1.51 | 577.236 | 578.3 | A |
| 371 | 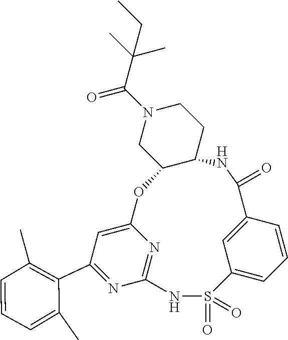 [SMILES: CCC(C)(C)C(=O)N1CC[C@@H]2NC(=O)c3cccc(c3)S(=O)(=O)Nc3nc(cc(-c4c(C)cccc4C)n3)O[C@@H]2C1] | 1.41 | 577.236 | 578.3 | A |
| 372 | 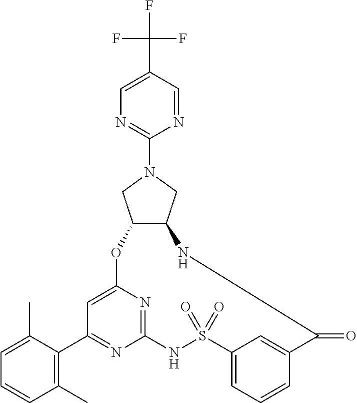 [SMILES: Cc1cccc(C)c1-c1cc2nc(n1)NS(=O)(=O)c1cccc(c1)C(=O)N[C@@H]1CN(c3ncc(C(F)(F)F)cn3)C[C@H]1O2] | 1.58 | 611.156 | 612.2 | A |
| 373 | 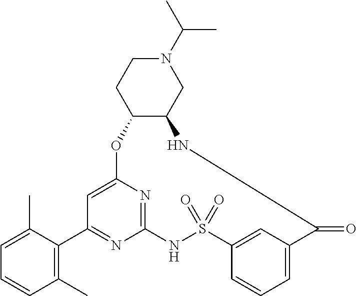 [SMILES: Cc1cccc(C)c1-c1cc2nc(n1)NS(=O)(=O)c1cccc(c1)C(=O)N[C@@H]1CN(C(C)C)CC[C@H]1O2] | 0.79 | 521.21 | 522.2 | A |
| 374 | 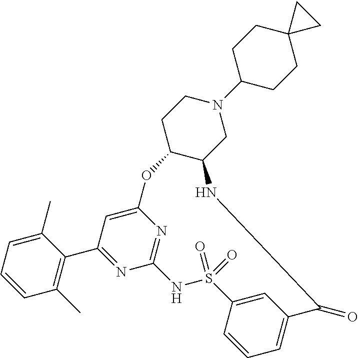 [SMILES: Cc1cccc(C)c1-c1cc2nc(n1)NS(=O)(=O)c1cccc(c1)C(=O)N[C@@H]1CN(C3CCC4(CC3)CC4)CC[C@H]1O2] | 1.14 | 587.257 | 588.3 | A |
| 375 | 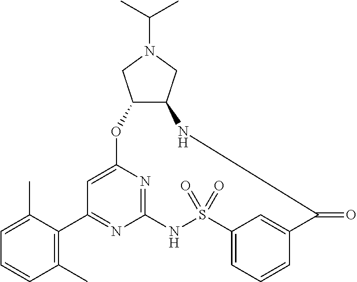 [SMILES: Cc1cccc(C)c1-c1cc2nc(n1)NS(=O)(=O)c1cccc(c1)C(=O)N[C@@H]1CN(C(C)C)C[C@H]1O2] | 0.76 | 507.194 | 508.2 | A |
| 376 | 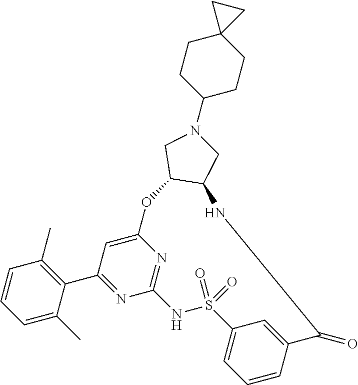 [SMILES: Cc1cccc(C)c1-c1cc2nc(n1)NS(=O)(=O)c1cccc(c1)C(=O)N[C@@H]1CN(C3CCC4(CC3)CC4)C[C@H]1O2] | 1.09 | 573.241 | 574.3 | A |
| 377 | 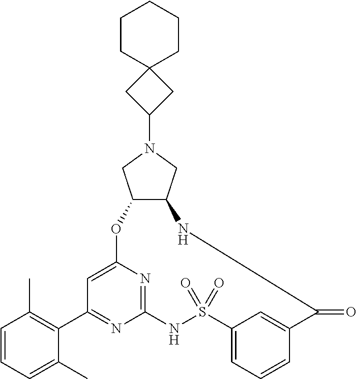 [SMILES: Cc1cccc(C)c1-c1cc2nc(n1)NS(=O)(=O)c1cccc(c1)C(=O)N[C@@H]1CN(C3CC4(CCCCC4)C3)C[C@H]1O2] | 1.27 | 587.257 | 588.4 | A |
| 378 | 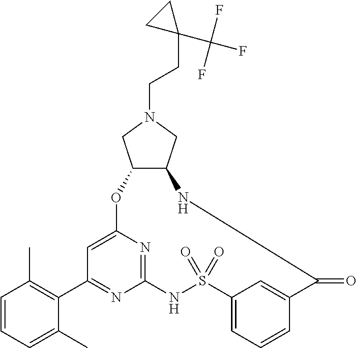 [SMILES: Cc1cccc(C)c1-c1cc2nc(n1)NS(=O)(=O)c1cccc(c1)C(=O)N[C@@H]1CN(CCC3(C(F)(F)F)CC3)C[C@H]1O2] | 1.1 | 601.197 | 602.3 | A |
| 379 | 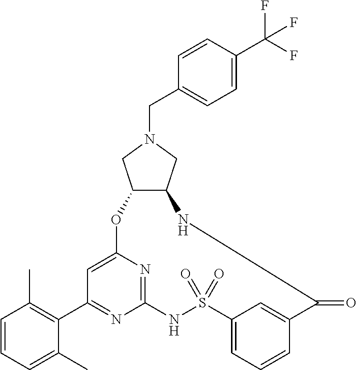 [SMILES: Cc1cccc(C)c1-c1cc2nc(n1)NS(=O)(=O)c1cccc(c1)C(=O)N[C@@H]1CN(Cc3ccc(C(F)(F)F)cc3)C[C@H]1O2] | 1.22 | 623.181 | 624.3 | A |
| 380 | 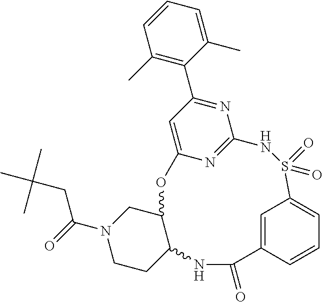 [SMILES: Cc1cccc(C)c1-c1cc2nc(n1)NS(=O)(=O)c1cccc(c1)C(=O)NC1CCN(C(=O)CC(C)(C)C)CC1O2] | 1.41 | 577.236 | 578.3 | A |
| 381 | 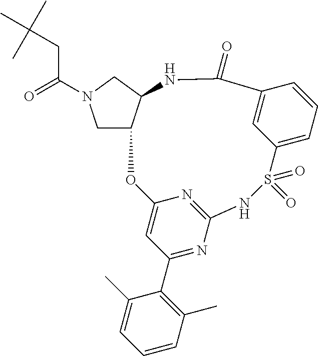 [SMILES: Cc1cccc(C)c1-c1cc2nc(n1)NS(=O)(=O)c1cccc(c1)C(=O)N[C@@H]1CN(C(=O)CC(C)(C)C)C[C@H]1O2] | 1.41 | 563.22 | 564.2 | A |
| 382 | 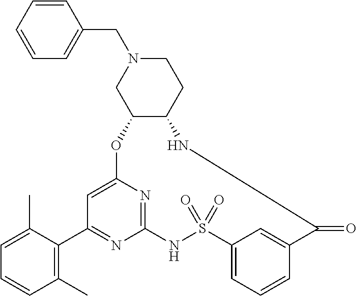 [SMILES: Cc1cccc(C)c1-c1cc2nc(n1)NS(=O)(=O)c1cccc(c1)C(=O)N[C@H]1CCN(Cc3ccccc3)C[C@H]1O2] | 0.93 | 569.21 | 570.3 | A |
| 383 | 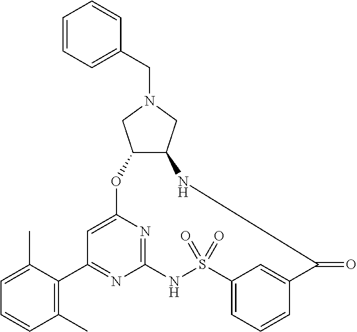 [SMILES: Cc1cccc(C)c1-c1cc2nc(n1)NS(=O)(=O)c1cccc(c1)C(=O)N[C@@H]1CN(Cc3ccccc3)C[C@H]1O2] | 1 | 555.194 | 556.3 | A |
| 57 | 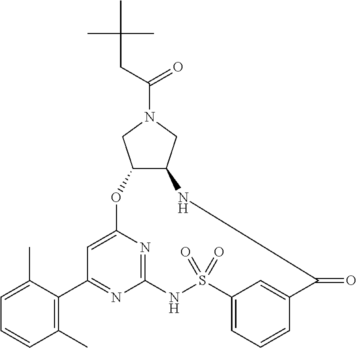 [SMILES: Cc1cccc(C)c1-c1cc2nc(n1)NS(=O)(=O)c1cccc(c1)C(=O)N[C@@H]1CN(C(=O)CC(C)(C)C)C[C@H]1O2] | 1.5 | 565.2 | 566.3 | A |
| 384 | 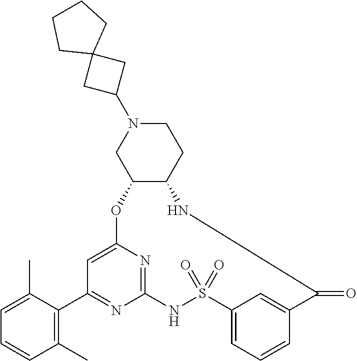 [SMILES: Cc1cccc(C)c1-c1cc2nc(n1)NS(=O)(=O)c1cccc(c1)C(=O)N[C@H]1CCN(C3CC4(CCCC4)C3)C[C@H]1O2] | 1.12 | 587.257 | 588.2 | A |
| 385 | 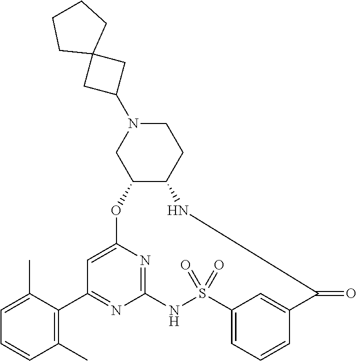 [SMILES: Cc1cccc(C)c1-c1cc2nc(n1)NS(=O)(=O)c1cccc(c1)C(=O)N[C@H]1CCN(C3CC4(CCCC4)C3)C[C@H]1O2] | 1.12 | 587.257 | 588.2 | A |
| 386 | 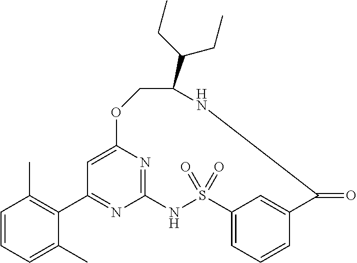 [SMILES: CCC(CC)[C@@H]1COc2cc(-c3c(C)cccc3C)nc(n2)NS(=O)(=O)c2cccc(c2)C(=O)N1] | 1.56 | 494.199 | 495.3 | A |
| 387 | 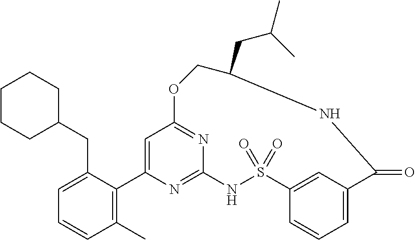 [SMILES: Cc1cccc(CC2CCCCC2)c1-c1cc2nc(n1)NS(=O)(=O)c1cccc(c1)C(=O)N[C@H](CC(C)C)CO2] | 1.93 | 562.261 | 563.2 | A |
| 388 | 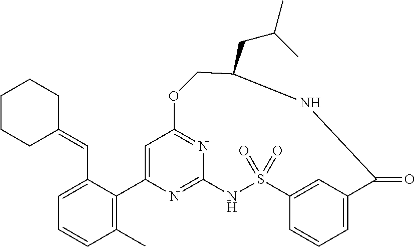 [SMILES: Cc1cccc(C=C2CCCCC2)c1-c1cc2nc(n1)NS(=O)(=O)c1cccc(c1)C(=O)N[C@H](CC(C)C)CO2] | 1.87 | 560.246 | 561.2 | A |
| 389 | 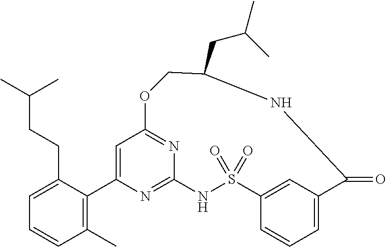 [SMILES: Cc1cccc(CCC(C)C)c1-c1cc2nc(n1)NS(=O)(=O)c1cccc(c1)C(=O)N[C@H](CC(C)C)CO2] | 1.86 | 536.246 | 537.2 | A |
| 390 | 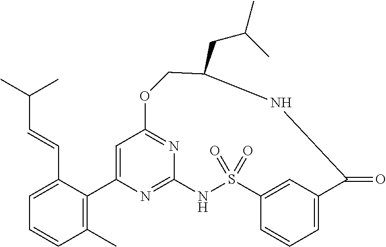 [SMILES: Cc1cccc(/C=C/C(C)C)c1-c1cc2nc(n1)NS(=O)(=O)c1cccc(c1)C(=O)N[C@H](CC(C)C)CO2] | 1.81 | 534.23 | 535.2 | A |
| 391 | 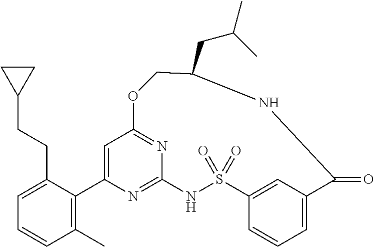 [SMILES: Cc1cccc(CCC2CC2)c1-c1cc2nc(n1)NS(=O)(=O)c1cccc(c1)C(=O)N[C@H](CC(C)C)CO2] | 1.75 | 534.23 | 535.2 | A |
| 53 | 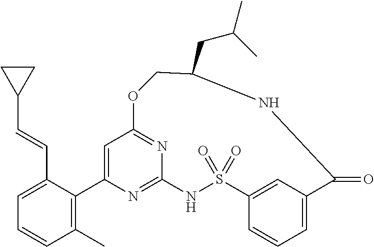 [SMILES: Cc1cccc(/C=C/C2CC2)c1-c1cc2nc(n1)NS(=O)(=O)c1cccc(c1)C(=O)N[C@H](CC(C)C)CO2] | 1.7 | 532.214 | 533.2 | A |
| 392 | 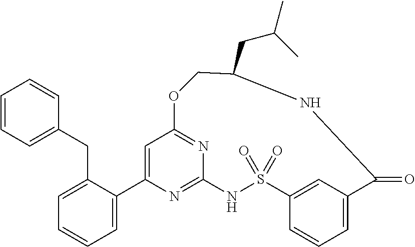 [SMILES: CC(C)C[C@@H]1COc2cc(-c3ccccc3Cc3ccccc3)nc(n2)NS(=O)(=O)c2cccc(c2)C(=O)N1] | 1.82 | 544.178 | 545.2 | A |
| 393 | 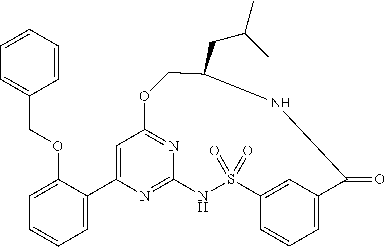 [SMILES: CC(C)C[C@@H]1COc2cc(-c3ccccc3OCc3ccccc3)nc(n2)NS(=O)(=O)c2cccc(c2)C(=O)N1] | 1.66 | 558.194 | 559.2 | A |
| 394 | 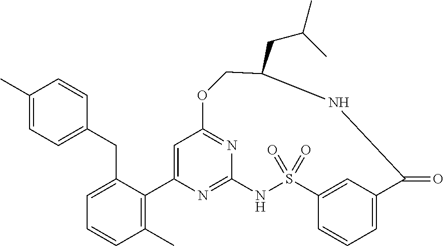 [SMILES: Cc1ccc(Cc2cccc(C)c2-c2cc3nc(n2)NS(=O)(=O)c2cccc(c2)C(=O)N[C@H](CC(C)C)CO3)cc1] | 1.8 | 570.23 | 571.2 | A |
| 395 | 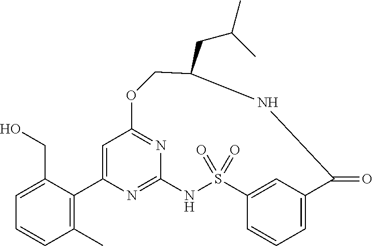 [SMILES: Cc1cccc(CO)c1-c1cc2nc(n1)NS(=O)(=O)c1cccc(c1)C(=O)N[C@H](CC(C)C)CO2] | 1.21 | 496.178 | 497.2 | A |
| 396 | 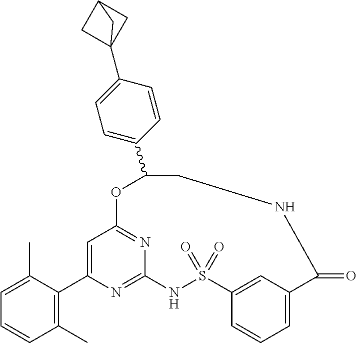 [SMILES: Cc1cccc(C)c1-c1cc2nc(n1)NS(=O)(=O)c1cccc(c1)C(=O)NCC(c1ccc(C34CC(C3)C4)cc1)O2] | 1.85 | 566.199 | 567.2 | A |
| 397 | 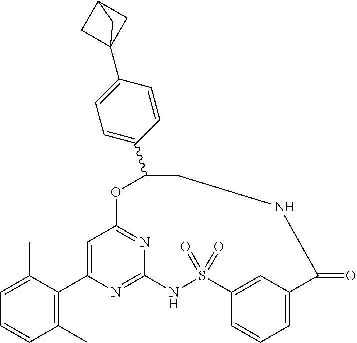 [SMILES: Cc1cccc(C)c1-c1cc2nc(n1)NS(=O)(=O)c1cccc(c1)C(=O)NCC(c1ccc(C34CC(C3)C4)cc1)O2] | 1.85 | 566.199 | 567.2 | A |
| 398 | 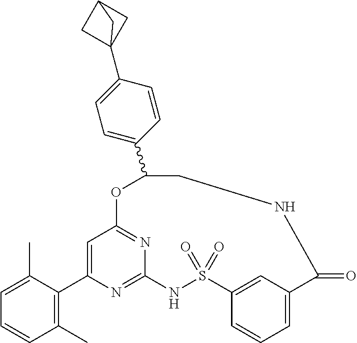 [SMILES: Cc1cccc(C)c1-c1cc2nc(n1)NS(=O)(=O)c1cccc(c1)C(=O)NCC(c1ccc(C34CC(C3)C4)cc1)O2] | 1.88 | 566.199 | 567.2 | A |
| 399 | 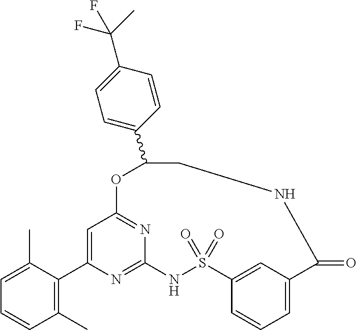 [SMILES: Cc1cccc(C)c1-c1cc2nc(n1)NS(=O)(=O)c1cccc(c1)C(=O)NCC(c1ccc(C(C)(F)F)cc1)O2] | 1.61 | 564.164 | 565.2 | A |
| 400 | 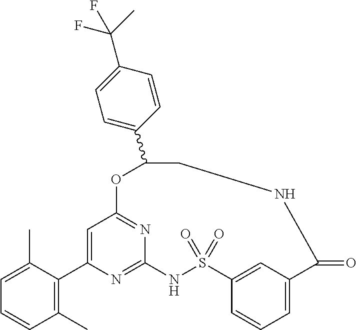 [SMILES: Cc1cccc(C)c1-c1cc2nc(n1)NS(=O)(=O)c1cccc(c1)C(=O)NCC(c1ccc(C(C)(F)F)cc1)O2] | 1.61 | 564.164 | 565.2 | A |
| 34 | 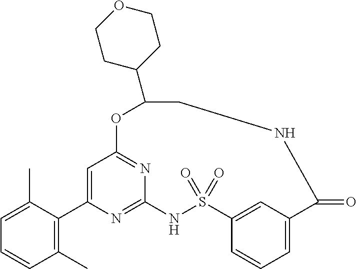 [SMILES: Cc1cccc(C)c1-c1cc2nc(n1)NS(=O)(=O)c1cccc(c1)C(=O)NCC(C1CCOCC1)O2] | 1.18 | 508.178 | 509.3 | A |
| 401 | 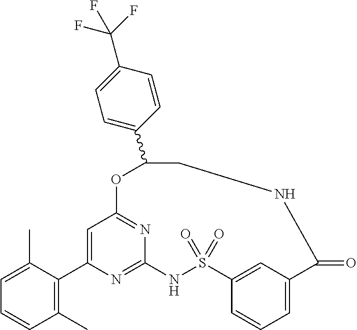 [SMILES: Cc1cccc(C)c1-c1cc2nc(n1)NS(=O)(=O)c1cccc(c1)C(=O)NCC(c1ccc(C(F)(F)F)cc1)O2] | 1.66 | 568.139 | 569.4 | A |
| 402 | 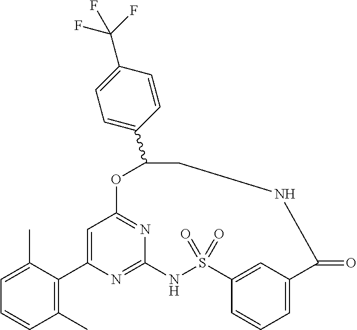 [SMILES: Cc1cccc(C)c1-c1cc2nc(n1)NS(=O)(=O)c1cccc(c1)C(=O)NCC(c1ccc(C(F)(F)F)cc1)O2] | 1.66 | 568.139 | 569.4 | A |
| 403 | 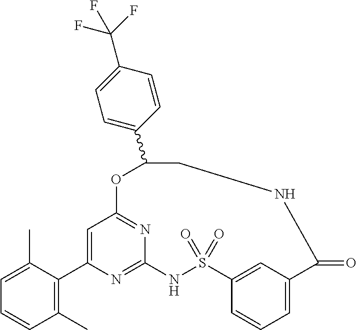 [SMILES: Cc1cccc(C)c1-c1cc2nc(n1)NS(=O)(=O)c1cccc(c1)C(=O)NCC(c1ccc(C(F)(F)F)cc1)O2] | 1.66 | 568.139 | 569.3 | A |
| 404 | 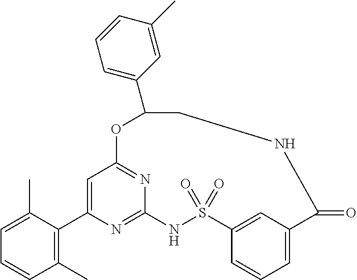 [SMILES: Cc1cccc(C2CNC(=O)c3cccc(c3)S(=O)(=O)Nc3nc(cc(-c4c(C)cccc4C)n3)O2)c1] | 1.53 | 514.167 | 515.3 | A |
| 405 | 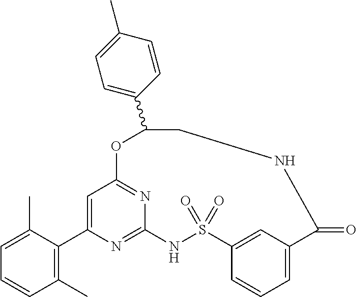 [SMILES: Cc1ccc(C2CNC(=O)c3cccc(c3)S(=O)(=O)Nc3nc(cc(-c4c(C)cccc4C)n3)O2)cc1] | 1.53 | 514.167 | 515.3 | A |
| 406 | 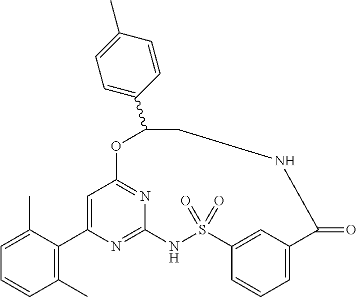 [SMILES: Cc1ccc(C2CNC(=O)c3cccc(c3)S(=O)(=O)Nc3nc(cc(-c4c(C)cccc4C)n3)O2)cc1] | 1.53 | 514.167 | 515.3 | A |
| 407 | 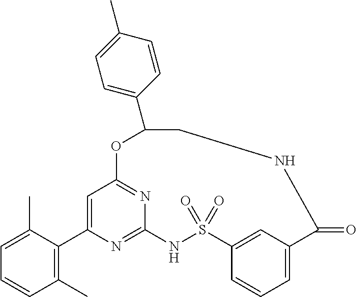 [SMILES: Cc1ccc(C2CNC(=O)c3cccc(c3)S(=O)(=O)Nc3nc(cc(-c4c(C)cccc4C)n3)O2)cc1] | 1.53 | 514.167 | 515.3 | A |
| 30 | 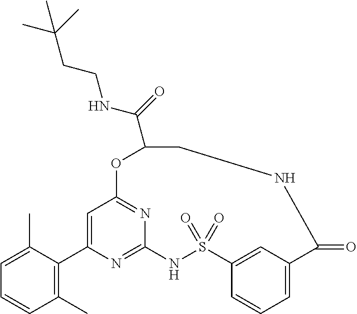 [SMILES: Cc1cccc(C)c1-c1cc2nc(n1)NS(=O)(=O)c1cccc(c1)C(=O)NCC(C(=O)NCCC(C)(C)C)O2] | 1.53 | 551.22 | 552.4 | A |
| 408 | 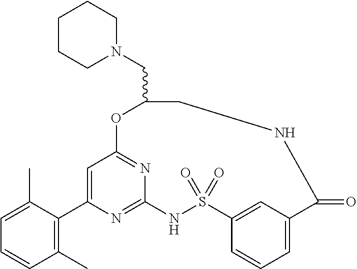 [SMILES: Cc1cccc(C)c1-c1cc2nc(n1)NS(=O)(=O)c1cccc(c1)C(=O)NCC(CN1CCCCC1)O2] | 0.84 | 521.21 | 522.4 | A |
| 409 | 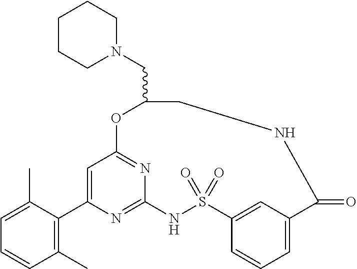 [SMILES: Cc1cccc(C)c1-c1cc2nc(n1)NS(=O)(=O)c1cccc(c1)C(=O)NCC(CN1CCCCC1)O2] | 0.84 | 521.21 | 522.4 | A |
| 410 | 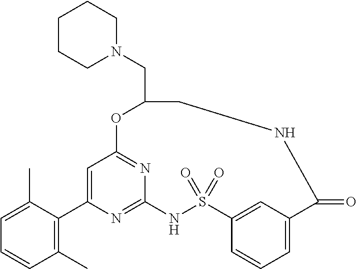 [SMILES: Cc1cccc(C)c1-c1cc2nc(n1)NS(=O)(=O)c1cccc(c1)C(=O)NCC(CN1CCCCC1)O2] | 0.84 | 521.21 | 522.2 | A |
| 411 | 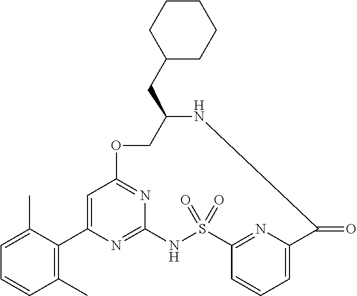 [SMILES: Cc1cccc(C)c1-c1cc2nc(n1)NS(=O)(=O)c1cccc(n1)C(=O)N[C@H](CC1CCCCC1)CO2] | 1.83 | 521.21 | 522 | A |
| 412 | 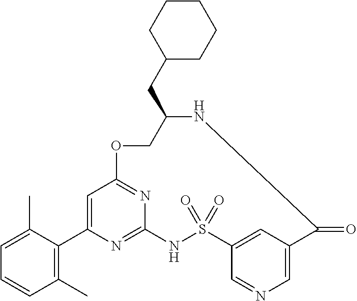 [SMILES: Cc1cccc(C)c1-c1cc2nc(n1)NS(=O)(=O)c1cncc(c1)C(=O)N[C@H](CC1CCCCC1)CO2] | 1.57 | 521.21 | 522 | A |
| 413 | 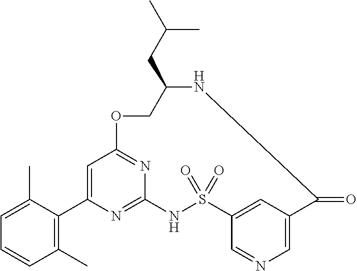 [SMILES: Cc1cccc(C)c1-c1cc2nc(n1)NS(=O)(=O)c1cncc(c1)C(=O)N[C@H](CC(C)C)CO2] | 1.36 | 481.178 | 482 | A |
| 414 | 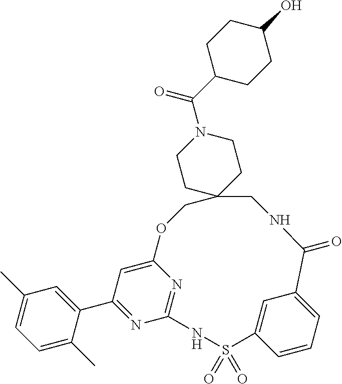 | 1.29 | 633.262 | 634 | A |
| 415 | 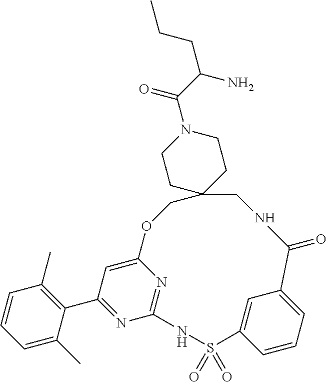 [SMILES: CCCC(N)C(=O)N1CCC2(CC1)CNC(=O)c1cccc(c1)S(=O)(=O)Nc1nc(cc(-c3c(C)cccc3C)n1)OC2] | 0.98 | 606.262 | 607 | A |
| 416 | 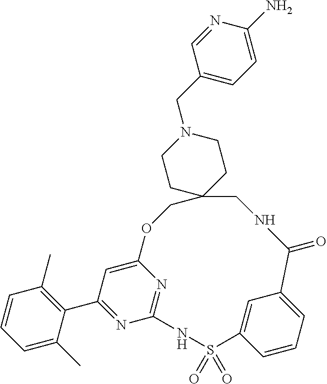 [SMILES: Cc1cccc(C)c1-c1cc2nc(n1)NS(=O)(=O)c1cccc(c1)C(=O)NCC1(CCN(Cc3ccc(N)nc3)CC1)CO2] | 0.75 | 613.247 | 614 | A |
| 417 | 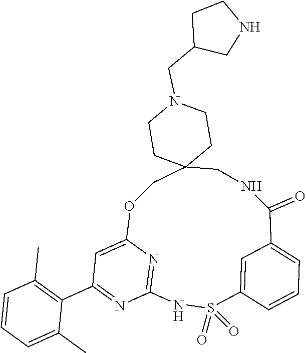 [SMILES: Cc1cccc(C)c1-c1cc2nc(n1)NS(=O)(=O)c1cccc(c1)C(=O)NCC1(CCN(CC3CCNC3)CC1)CO2] | 0.71 | 590.268 | 591 | A |
| 418 | 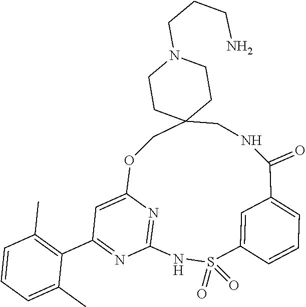 [SMILES: Cc1cccc(C)c1-c1cc2nc(n1)NS(=O)(=O)c1cccc(c1)C(=O)NCC1(CCN(CCCN)CC1)CO2] | 0.69 | 564.252 | 565 | A |
| 419 | 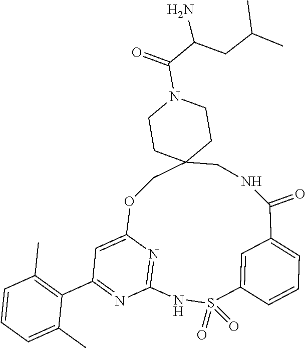 [SMILES: Cc1cccc(C)c1-c1cc2nc(n1)NS(=O)(=O)c1cccc(c1)C(=O)NCC1(CCN(C(=O)C(N)CC(C)C)CC1)CO2] | 1.06 | 620.278 | 621 | A |
| 420 | 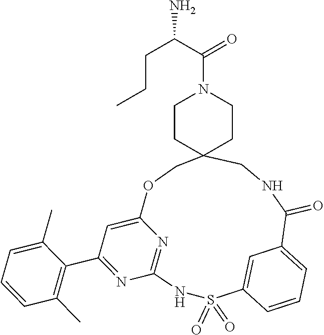 [SMILES: CCC[C@H](N)C(=O)N1CCC2(CC1)CNC(=O)c1cccc(c1)S(=O)(=O)Nc1nc(cc(-c3c(C)cccc3C)n1)OC2] | 0.99 | 606.262 | 607 | A |
| 421 | 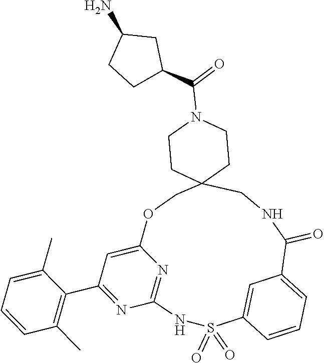 [SMILES: Cc1cccc(C)c1-c1cc2nc(n1)NS(=O)(=O)c1cccc(c1)C(=O)NCC1(CCN(C(=O)[C@H]3CC[C@@H](N)C3)CC1)CO2] | 0.92 | 618.262 | 619 | A |
| 422 | 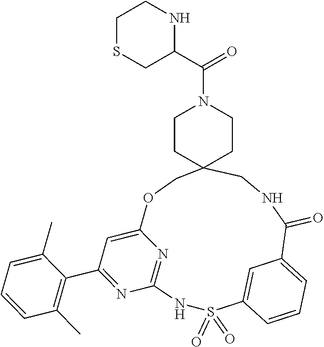 [SMILES: Cc1cccc(C)c1-c1cc2nc(n1)NS(=O)(=O)c1cccc(c1)C(=O)NCC1(CCN(C(=O)C3CSCCN3)CC1)CO2] | 0.91 | 636.219 | 637 | A |
| 423 | 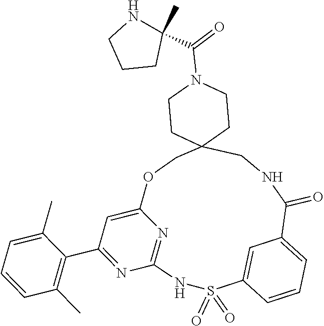 [SMILES: Cc1cccc(C)c1-c1cc2nc(n1)NS(=O)(=O)c1cccc(c1)C(=O)NCC1(CCN(C(=O)[C@]3(C)CCCN3)CC1)CO2] | 0.93 | 618.262 | 619 | A |
| 424 | 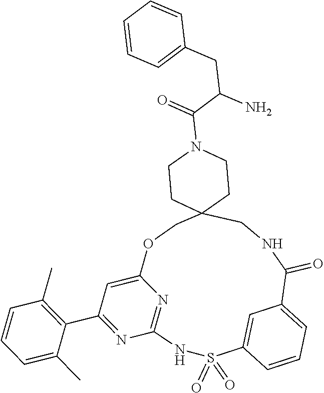 [SMILES: Cc1cccc(C)c1-c1cc2nc(n1)NS(=O)(=O)c1cccc(c1)C(=O)NCC1(CCN(C(=O)C(N)Cc3ccccc3)CC1)CO2] | 1.28 | 654.262 | 655 | A |
| 425 | 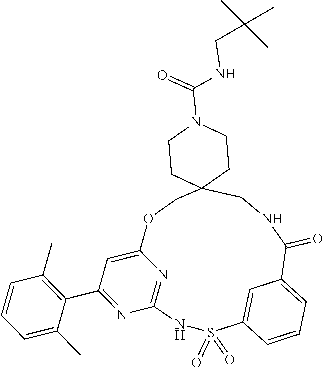 [SMILES: Cc1cccc(C)c1-c1cc2nc(n1)NS(=O)(=O)c1cccc(c1)C(=O)NCC1(CCN(C(=O)NCC(C)(C)C)CC1)CO2] | 1.56 | 620.278 | 621 | A |
| 426 | 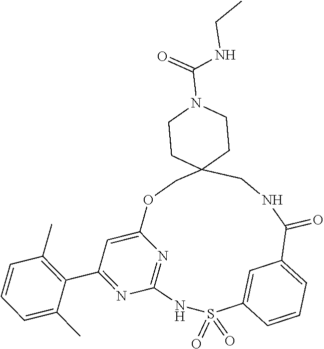 [SMILES: CCNC(=O)N1CCC2(CC1)CNC(=O)c1cccc(c1)S(=O)(=O)Nc1nc(cc(-c3c(C)cccc3C)n1)OC2] | 1.28 | 578.231 | 579 | A |
| 427 | 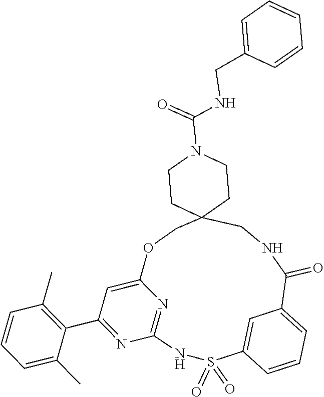 [SMILES: Cc1cccc(C)c1-c1cc2nc(n1)NS(=O)(=O)c1cccc(c1)C(=O)NCC1(CCN(C(=O)NCc3ccccc3)CC1)CO2] | 1.62 | 640.247 | 641 | A |
| 428 | 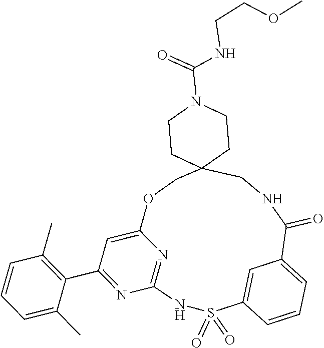 [SMILES: COCCNC(=O)N1CCC2(CC1)CNC(=O)c1cccc(c1)S(=O)(=O)Nc1nc(cc(-c3c(C)cccc3C)n1)OC2] | 1.23 | 608.242 | 609 | A |
| 429 | 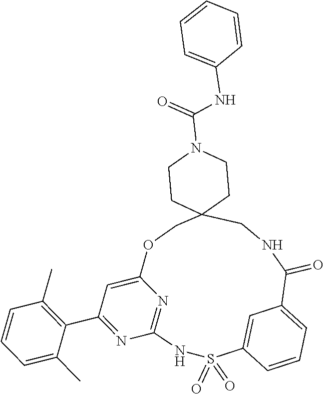 [SMILES: Cc1cccc(C)c1-c1cc2nc(n1)NS(=O)(=O)c1cccc(c1)C(=O)NCC1(CCN(C(=O)Nc3ccccc3)CC1)CO2] | 1.41 | 626.231 | 627 | A |
| 430 | 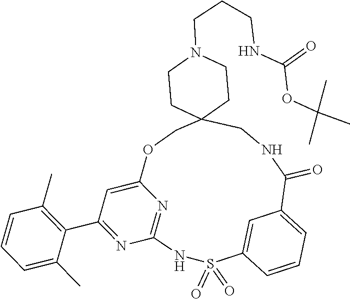 [SMILES: Cc1cccc(C)c1-c1cc2nc(n1)NS(=O)(=O)c1cccc(c1)C(=O)NCC1(CCN(CCCNC(=O)OC(C)(C)C)CC1)CO2] | 1.15 | 664.304 | 665 | A |
| 431 | 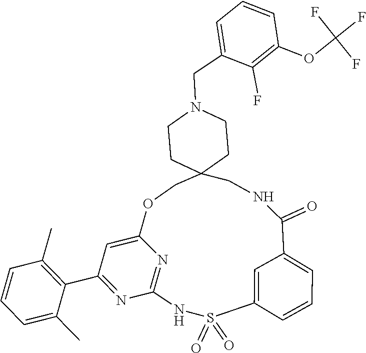 [SMILES: Cc1cccc(C)c1-c1cc2nc(n1)NS(=O)(=O)c1cccc(c1)C(=O)NCC1(CCN(Cc3cccc(OC(F)(F)F)c3F)CC1)CO2] | 1.29 | 699.214 | 700 | A |
| 432 | 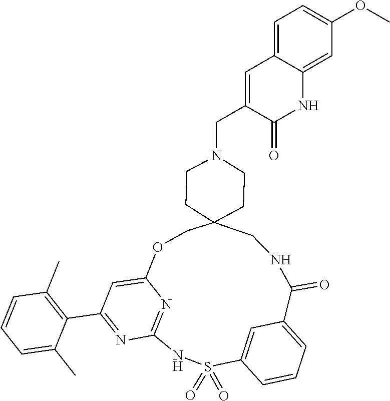 [SMILES: COc1ccc2cc(CN3CCC4(CC3)CNC(=O)c3cccc(c3)S(=O)(=O)Nc3nc(cc(-c5c(C)cccc5C)n3)OC4)c(=O)[nH]c2c1] | 1.06 | 694.257 | 695 | A |
| 433 | 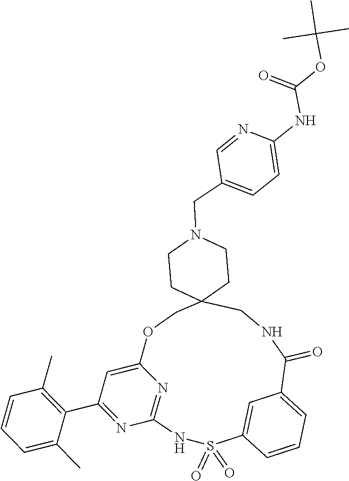 [SMILES: Cc1cccc(C)c1-c1cc2nc(n1)NS(=O)(=O)c1cccc(c1)C(=O)NCC1(CCN(Cc3ccc(NC(=O)OC(C)(C)C)nc3)CC1)CO2] | 1.24 | 713.3 | 714 | A |
| 434 | 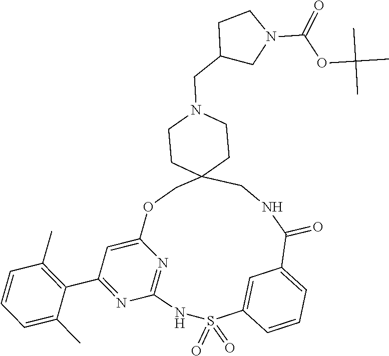 [SMILES: Cc1cccc(C)c1-c1cc2nc(n1)NS(=O)(=O)c1cccc(c1)C(=O)NCC1(CCN(CC3CCN(C(=O)OC(C)(C)C)C3)CC1)CO2] | 1.16 | 690.32 | 691 | A |
| 435 | 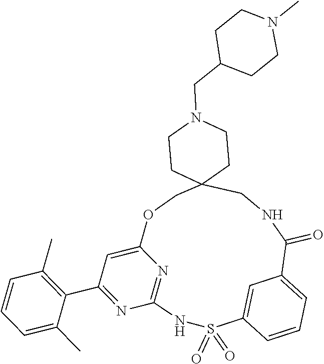 [SMILES: Cc1cccc(C)c1-c1cc2nc(n1)NS(=O)(=O)c1cccc(c1)C(=O)NCC1(CCN(CC3CCN(C)CC3)CC1)CO2] | 0.68 | 618.299 | 619 | A |
| 436 | 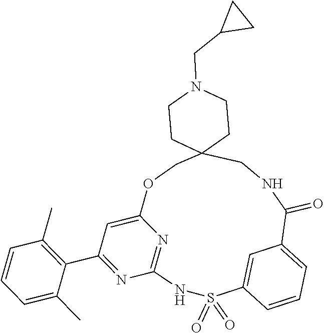 [SMILES: Cc1cccc(C)c1-c1cc2nc(n1)NS(=O)(=O)c1cccc(c1)C(=O)NCC1(CCN(CC3CC3)CC1)CO2] | 0.94 | 561.241 | 562 | A |
| 437 | 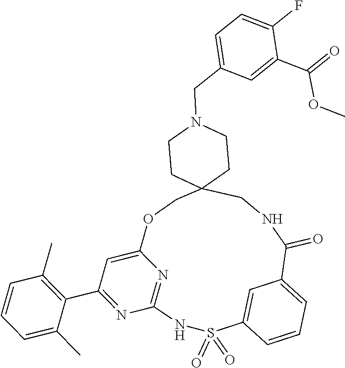 [SMILES: COC(=O)c1cc(CN2CCC3(CC2)CNC(=O)c2cccc(c2)S(=O)(=O)Nc2nc(cc(-c4c(C)cccc4C)n2)OC3)ccc1F] | 1.07 | 673.237 | 674 | A |
| 438 | 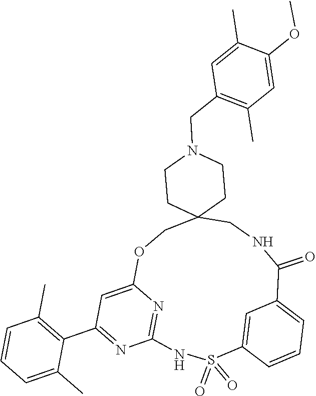 [SMILES: COc1cc(C)c(CN2CCC3(CC2)CNC(=O)c2cccc(c2)S(=O)(=O)Nc2nc(cc(-c4c(C)cccc4C)n2)OC3)cc1C] | 1.25 | 655.283 | 656 | A |
| 439 | 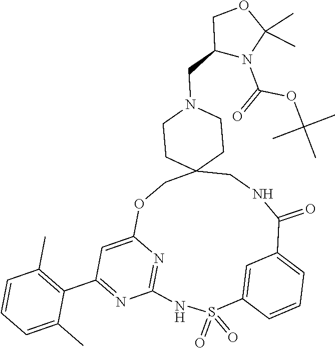 [SMILES: Cc1cccc(C)c1-c1cc2nc(n1)NS(=O)(=O)c1cccc(c1)C(=O)NCC1(CCN(C[C@H]3COC(C)(C)N3C(=O)OC(C)(C)C)CC1)CO2] | 1.36 | 720.331 | 721 | A |
| 440 | 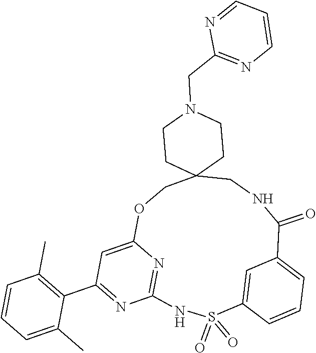 [SMILES: Cc1cccc(C)c1-c1cc2nc(n1)NS(=O)(=O)c1cccc(c1)C(=O)NCC1(CCN(Cc3ncccn3)CC1)CO2] | 0.86 | 599.231 | 600 | A |
| 441 | 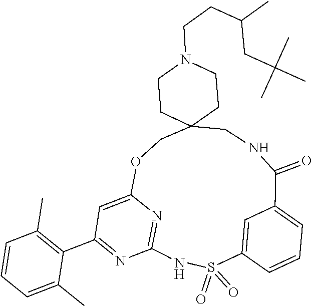 [SMILES: Cc1cccc(C)c1-c1cc2nc(n1)NS(=O)(=O)c1cccc(c1)C(=O)NCC1(CCN(CCC(C)CC(C)(C)C)CC1)CO2] | 1.39 | 633.335 | 634 | A |
| 442 | 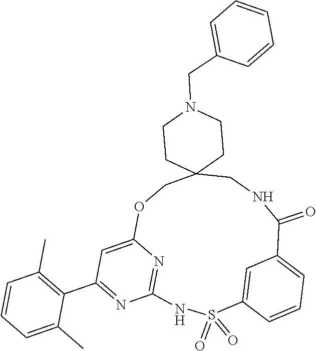 [SMILES: Cc1cccc(C)c1-c1cc2nc(n1)NS(=O)(=O)c1cccc(c1)C(=O)NCC1(CCN(Cc3ccccc3)CC1)CO2] | 1.07 | 597.241 | 598 | A |
| 443 | 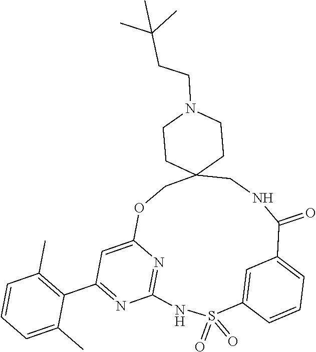 [SMILES: Cc1cccc(C)c1-c1cc2nc(n1)NS(=O)(=O)c1cccc(c1)C(=O)NCC1(CCN(CCC(C)(C)C)CC1)CO2] | 1.31 | 591.288 | 592 | A |
| 444 | 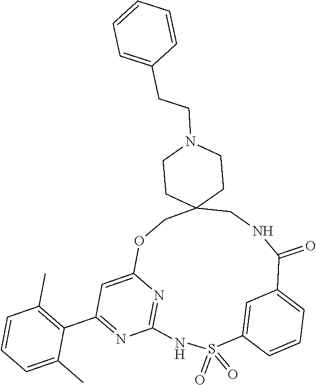 [SMILES: Cc1cccc(C)c1-c1cc2nc(n1)NS(=O)(=O)c1cccc(c1)C(=O)NCC1(CCN(CCc3ccccc3)CC1)CO2] | 1.31 | 611.257 | 612 | A |
| 445 | 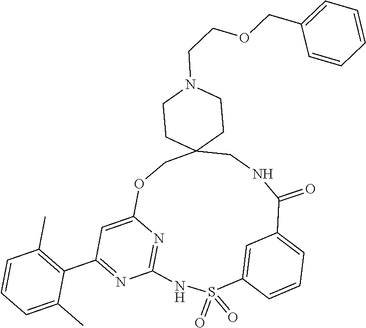 [SMILES: Cc1cccc(C)c1-c1cc2nc(n1)NS(=O)(=O)c1cccc(c1)C(=O)NCC1(CCN(CCOCc3ccccc3)CC1)CO2] | 1.33 | 641.267 | 642 | A |
| 446 | 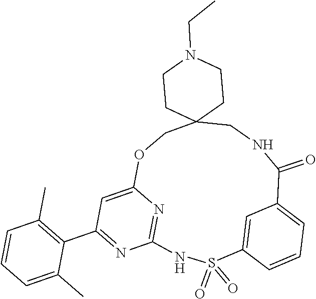 [SMILES: CCN1CCC2(CC1)CNC(=O)c1cccc(c1)S(=O)(=O)Nc1nc(cc(-c3c(C)cccc3C)n1)OC2] | 1 | 535.225 | 536 | A |
| 447 | 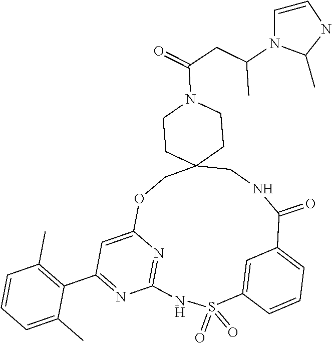 [SMILES: Cc1cccc(C)c1-c1cc2nc(n1)NS(=O)(=O)c1cccc(c1)C(=O)NCC1(CCN(C(=O)CC(C)N3C=CNC3C)CC1)CO2] | 0.92 | 657.273 | 658 | A |
| 448 | 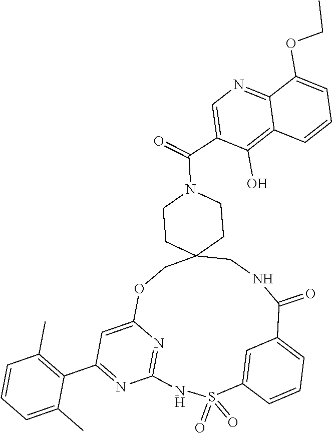 [SMILES: CCOc1cccc2c(O)c(C(=O)N3CCC4(CC3)CNC(=O)c3cccc(c3)S(=O)(=O)Nc3nc(cc(-c5c(C)cccc5C)n3)OC4)cnc12] | 1.28 | 722.252 | 723 | A |
| 449 | 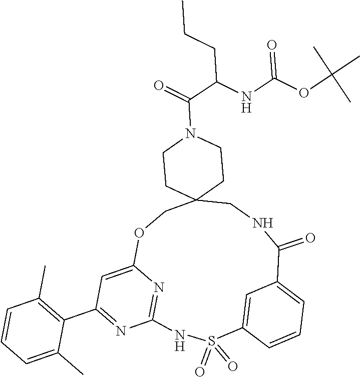 [SMILES: CCCC(NC(=O)OC(C)(C)C)C(=O)N1CCC2(CC1)CNC(=O)c1cccc(c1)S(=O)(=O)Nc1nc(cc(-c3c(C)cccc3C)n1)OC2] | 1.65 | 706.315 | 707 | A |
| 450 | 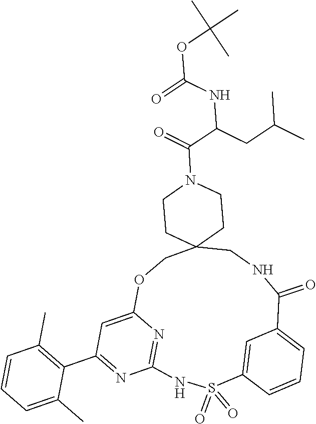 [SMILES: Cc1cccc(C)c1-c1cc2nc(n1)NS(=O)(=O)c1cccc(c1)C(=O)NCC1(CCN(C(=O)C(CC(C)C)NC(=O)OC(C)(C)C)CC1)CO2] | 1.74 | 720.331 | 721.22 | A |
| 451 | 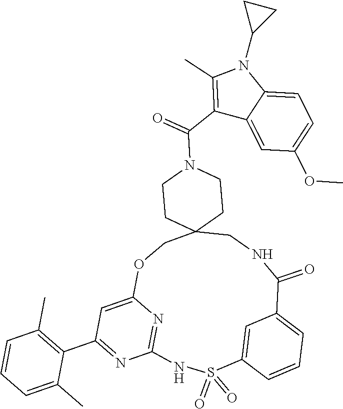 [SMILES: COc1ccc2c(c1)c(C(=O)N1CCC3(CC1)CNC(=O)c1cccc(c1)S(=O)(=O)Nc1nc(cc(-c4c(C)cccc4C)n1)OC3)c(C)n2C1CC1] | 1.68 | 734.289 | 735.12 | A |
| 452 | 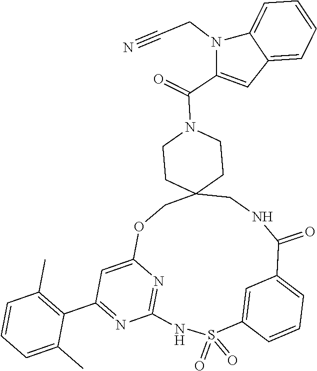 [SMILES: Cc1cccc(C)c1-c1cc2nc(n1)NS(=O)(=O)c1cccc(c1)C(=O)NCC1(CCN(C(=O)c3cc4ccccc4n3CC#N)CC1)CO2] | 1.68 | 689.242 | 690.07 | A |
| 453 | 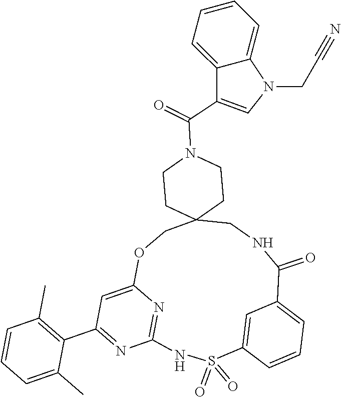 [SMILES: Cc1cccc(C)c1-c1cc2nc(n1)NS(=O)(=O)c1cccc(c1)C(=O)NCC1(CCN(C(=O)c3cn(CC#N)c4ccccc34)CC1)CO2] | 1.52 | 689.242 | 690.12 | A |
| 454 | 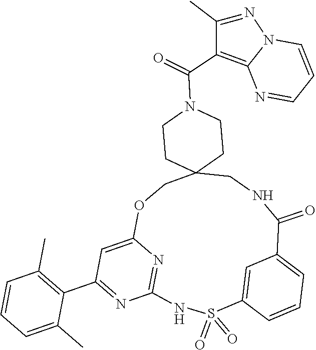 [SMILES: Cc1cccc(C)c1-c1cc2nc(n1)NS(=O)(=O)c1cccc(c1)C(=O)NCC1(CCN(C(=O)c3c(C)nn4cccnc34)CC1)CO2] | 1.14 | 666.237 | 667 | A |
| 455 | 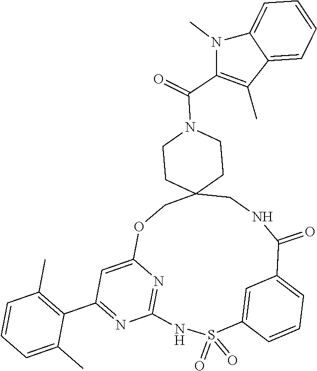 [SMILES: Cc1cccc(C)c1-c1cc2nc(n1)NS(=O)(=O)c1cccc(c1)C(=O)NCC1(CCN(C(=O)c3c(C)c4ccccc4n3C)CC1)CO2] | 1.72 | 678.262 | 679.07 | A |
| 456 | 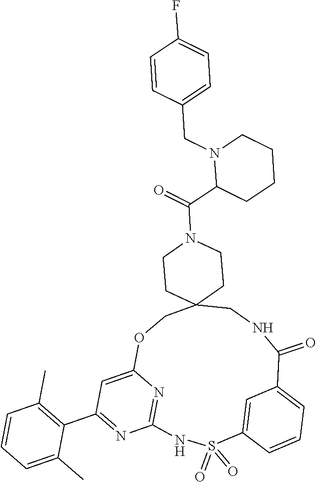 [SMILES: Cc1cccc(C)c1-c1cc2nc(n1)NS(=O)(=O)c1cccc(c1)C(=O)NCC1(CCN(C(=O)C3CCCCN3Cc3ccc(F)cc3)CC1)CO2] | 1.14 | 726.3 | 727 | A |
| 457 | 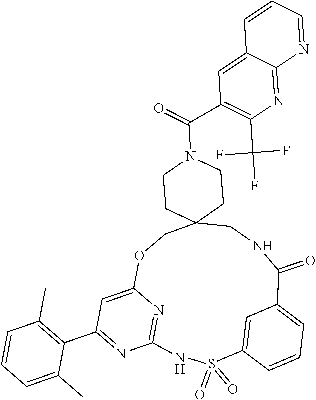 [SMILES: Cc1cccc(C)c1-c1cc2nc(n1)NS(=O)(=O)c1cccc(c1)C(=O)NCC1(CCN(C(=O)c3cc4cccnc4nc3C(F)(F)F)CC1)CO2] | 1.3 | 731.214 | 732 | A |
| 458 | 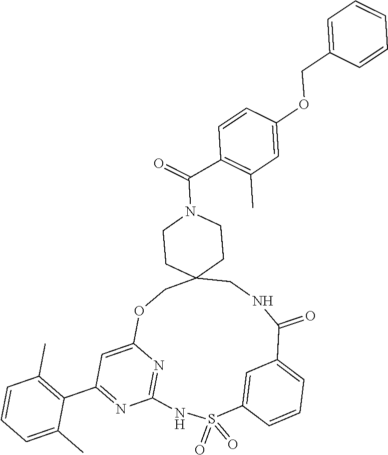 [SMILES: Cc1cc(OCc2ccccc2)ccc1C(=O)N1CCC2(CC1)CNC(=O)c1cccc(c1)S(=O)(=O)Nc1nc(cc(-c3c(C)cccc3C)n1)OC2] | 1.41 | 731.278 | 732.02 | A |
| 459 | 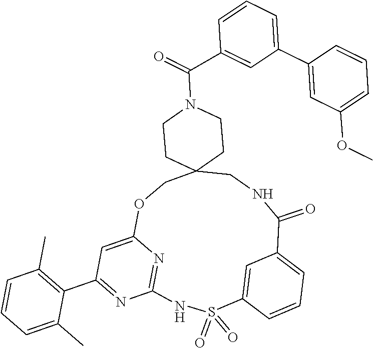 [SMILES: COc1cccc(-c2cccc(C(=O)N3CCC4(CC3)CNC(=O)c3cccc(c3)S(=O)(=O)Nc3nc(cc(-c5c(C)cccc5C)n3)OC4)c2)c1] | 1.75 | 717.262 | 718.12 | A |
| 460 | 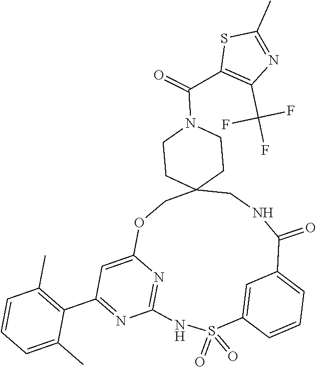 [SMILES: Cc1nc(C(F)(F)F)c(C(=O)N2CCC3(CC2)CNC(=O)c2cccc(c2)S(=O)(=O)Nc2nc(cc(-c4c(C)cccc4C)n2)OC3)s1] | 1.52 | 700.175 | 701.07 | A |
| 461 | 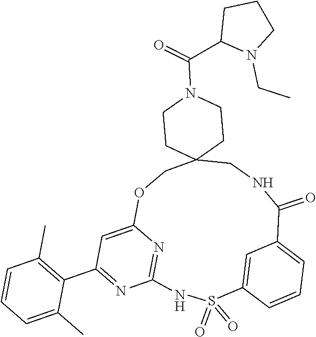 [SMILES: CCN1CCCC1C(=O)N1CCC2(CC1)CNC(=O)c1cccc(c1)S(=O)(=O)Nc1nc(cc(-c3c(C)cccc3C)n1)OC2] | 1.13 | 632.278 | 633.16 | A |
| 462 | 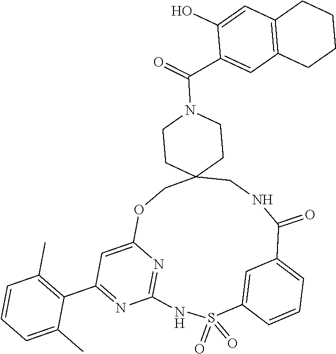 [SMILES: Cc1cccc(C)c1-c1cc2nc(n1)NS(=O)(=O)c1cccc(c1)C(=O)NCC1(CCN(C(=O)c3cc4c(cc3O)CCCC4)CC1)CO2] | 1.59 | 681.262 | 682 | A |
| 463 | 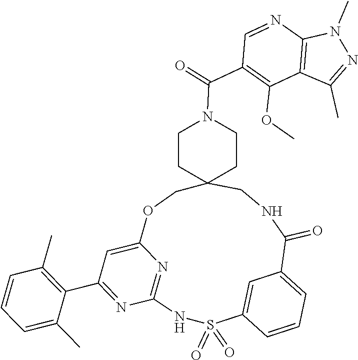 [SMILES: COc1c(C(=O)N2CCC3(CC2)CNC(=O)c2cccc(c2)S(=O)(=O)Nc2nc(cc(-c4c(C)cccc4C)n2)OC3)cnc2c1c(C)nn2C] | 1.4 | 710.264 | 711.12 | A |
| 464 | 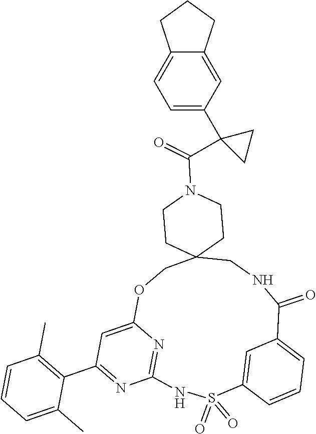 [SMILES: Cc1cccc(C)c1-c1cc2nc(n1)NS(=O)(=O)c1cccc(c1)C(=O)NCC1(CCN(C(=O)C3(c4ccc5c(c4)CCC5)CC3)CC1)CO2] | 1.76 | 691.283 | 692 | A |
| 465 | 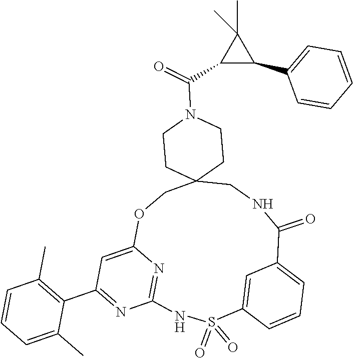 [SMILES: Cc1cccc(C)c1-c1cc2nc(n1)NS(=O)(=O)c1cccc(c1)C(=O)NCC1(CCN(C(=O)[C@H]3[C@H](c4ccccc4)C3(C)C)CC1)CO2] | 1.75 | 679.283 | 680 | A |
| 466 | 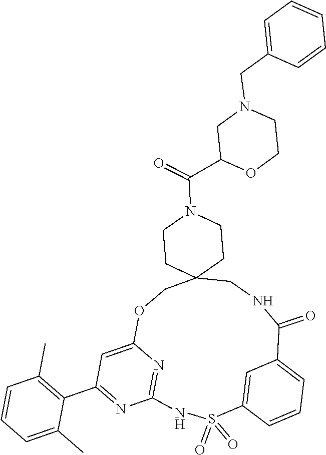 [SMILES: Cc1cccc(C)c1-c1cc2nc(n1)NS(=O)(=O)c1cccc(c1)C(=O)NCC1(CCN(C(=O)C3CN(Cc4ccccc4)CCO3)CC1)CO2] | 1.13 | 710.289 | 711 | A |
| 467 | 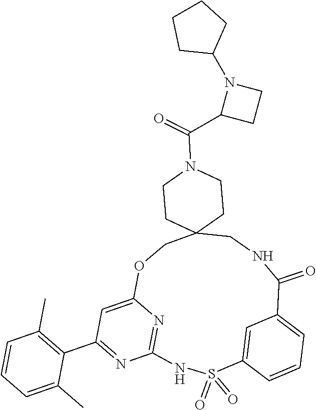 [SMILES: Cc1cccc(C)c1-c1cc2nc(n1)NS(=O)(=O)c1cccc(c1)C(=O)NCC1(CCN(C(=O)C3CCN3C3CCCC3)CC1)CO2] | 1.02 | 658.294 | 659 | A |
| 468 | 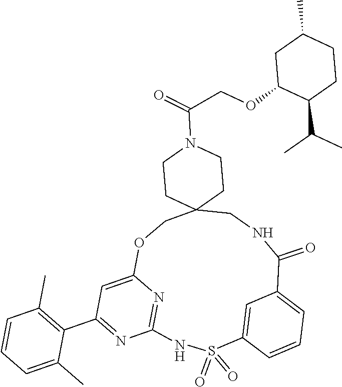 [SMILES: Cc1cccc(C)c1-c1cc2nc(n1)NS(=O)(=O)c1cccc(c1)C(=O)NCC1(CCN(C(=O)CO[C@@H]3C[C@H](C)CC[C@H]3C(C)C)CC1)CO2] | 1.98 | 703.34 | 704 | A |
| 469 | 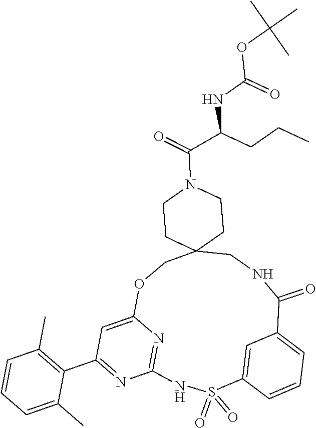 [SMILES: CCC[C@H](NC(=O)OC(C)(C)C)C(=O)N1CCC2(CC1)CNC(=O)c1cccc(c1)S(=O)(=O)Nc1nc(cc(-c3c(C)cccc3C)n1)OC2] | 1.89 | 706.315 | 707 | A |
| 470 | 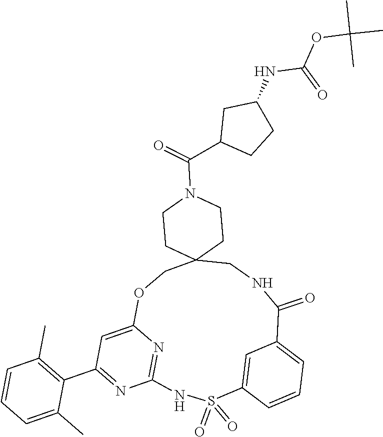 [SMILES: Cc1cccc(C)c1-c1cc2nc(n1)NS(=O)(=O)c1cccc(c1)C(=O)NCC1(CCN(C(=O)C3CC[C@@H](NC(=O)OC(C)(C)C)C3)CC1)CO2] | 1.84 | 718.315 | 719 | A |
| 471 | 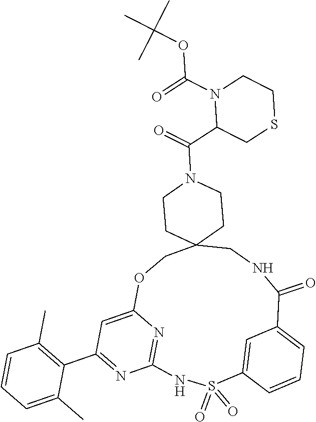 [SMILES: Cc1cccc(C)c1-c1cc2nc(n1)NS(=O)(=O)c1cccc(c1)C(=O)NCC1(CCN(C(=O)C3CSCCN3C(=O)OC(C)(C)C)CC1)CO2] | 1.8 | 736.271 | 737 | A |
| 472 | 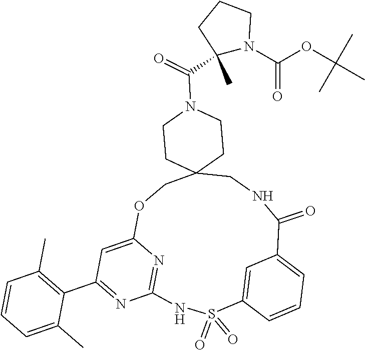 [SMILES: Cc1cccc(C)c1-c1cc2nc(n1)NS(=O)(=O)c1cccc(c1)C(=O)NCC1(CCN(C(=O)[C@]3(C)CCCN3C(=O)OC(C)(C)C)CC1)CO2] | 1.79 | 718.315 | 719 | A |
| 473 | 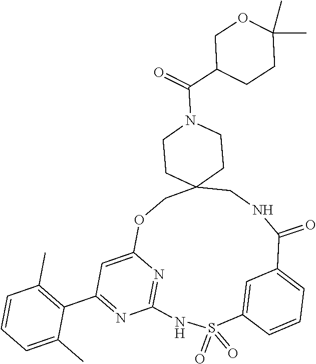 [SMILES: Cc1cccc(C)c1-c1cc2nc(n1)NS(=O)(=O)c1cccc(c1)C(=O)NCC1(CCN(C(=O)C3CCC(C)(C)OC3)CC1)CO2] | 1.47 | 647.278 | 648 | A |
| 474 | 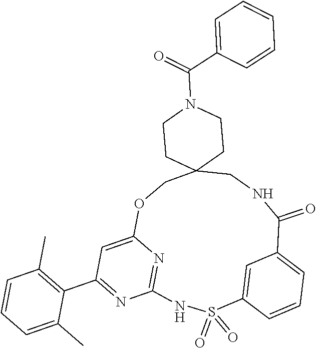 [SMILES: Cc1cccc(C)c1-c1cc2nc(n1)NS(=O)(=O)c1cccc(c1)C(=O)NCC1(CCN(C(=O)c3ccccc3)CC1)CO2] | 1.61 | 611.22 | 612 | A |
| 475 | 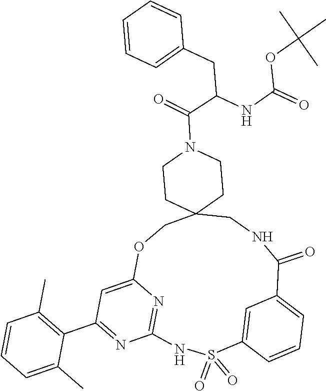 [SMILES: Cc1cccc(C)c1-c1cc2nc(n1)NS(=O)(=O)c1cccc(c1)C(=O)NCC1(CCN(C(=O)C(Cc3ccccc3)NC(=O)OC(C)(C)C)CC1)CO2] | 1.87 | 754.315 | 755 | A |
| 476 | 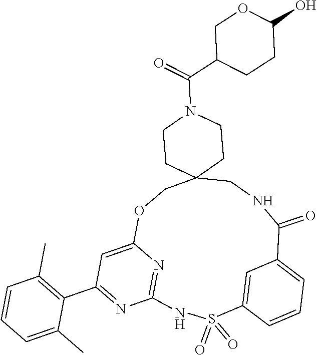 | 1.24 | 633.262 | 634 | A |
| 477 | 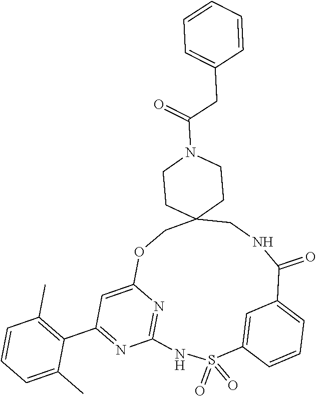 [SMILES: Cc1cccc(C)c1-c1cc2nc(n1)NS(=O)(=O)c1cccc(c1)C(=O)NCC1(CCN(C(=O)Cc3ccccc3)CC1)CO2] | 1.5 | 625.236 | 626 | A |
| 478 | 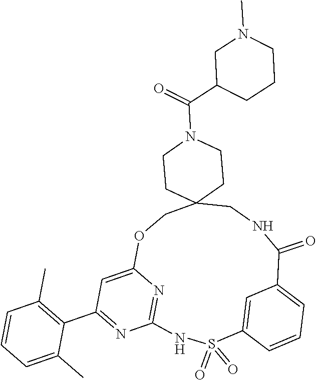 [SMILES: Cc1cccc(C)c1-c1cc2nc(n1)NS(=O)(=O)c1cccc(c1)C(=O)NCC1(CCN(C(=O)C3CCCN(C)C3)CC1)CO2] | 1.01 | 632.278 | 633 | A |
| 479 | 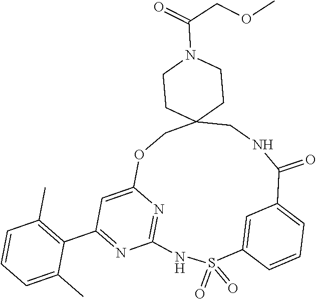 [SMILES: COCC(=O)N1CCC2(CC1)CNC(=O)c1cccc(c1)S(=O)(=O)Nc1nc(cc(-c3c(C)cccc3C)n1)OC2] | 1.23 | 579.215 | 580 | A |
| 480 | 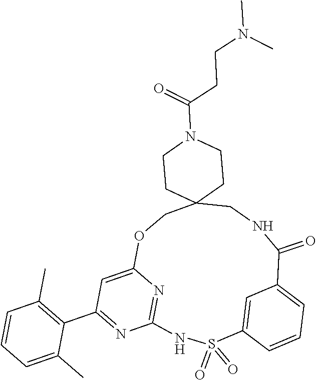 [SMILES: Cc1cccc(C)c1-c1cc2nc(n1)NS(=O)(=O)c1cccc(c1)C(=O)NCC1(CCN(C(=O)CCN(C)C)CC1)CO2] | 1.17 | 606.262 | 607 | A |
| 481 | 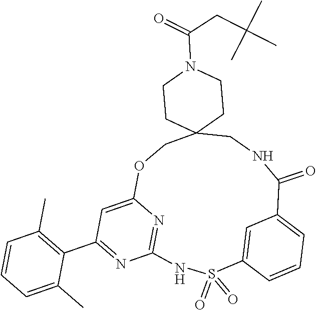 [SMILES: Cc1cccc(C)c1-c1cc2nc(n1)NS(=O)(=O)c1cccc(c1)C(=O)NCC1(CCN(C(=O)CC(C)(C)C)CC1)CO2] | 1.7 | 605.267 | 606 | A |
| 482 | 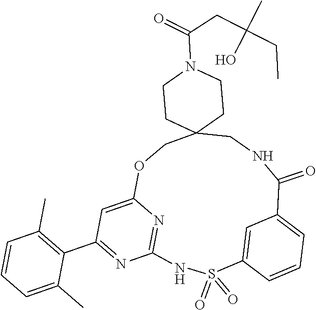 [SMILES: CCC(C)(O)CC(=O)N1CCC2(CC1)CNC(=O)c1cccc(c1)S(=O)(=O)Nc1nc(cc(-c3c(C)cccc3C)n1)OC2] | 1.38 | 621.262 | 622 | A |
| 483 | 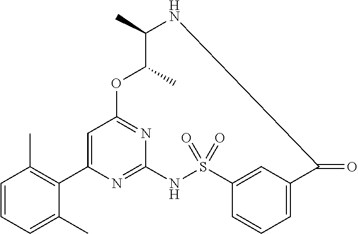 [SMILES: Cc1cccc(C)c1-c1cc2nc(n1)NS(=O)(=O)c1cccc(c1)C(=O)N[C@H](C)[C@H](C)O2] | 1.22 | 452.152 | 453.3 | A |
| 484 | 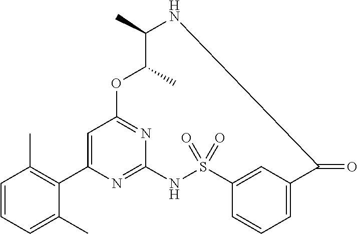 [SMILES: Cc1cccc(C)c1-c1cc2nc(n1)NS(=O)(=O)c1cccc(c1)C(=O)N[C@H](C)[C@H](C)O2] | 1.26 | 452.152 | 453.3 | A |
| 485 | 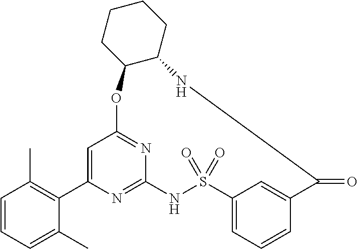 [SMILES: Cc1cccc(C)c1-c1cc2nc(n1)NS(=O)(=O)c1cccc(c1)C(=O)N[C@H]1CCCC[C@@H]1O2] | 1.49 | 478.167 | 479.3 | A |
| 486 | 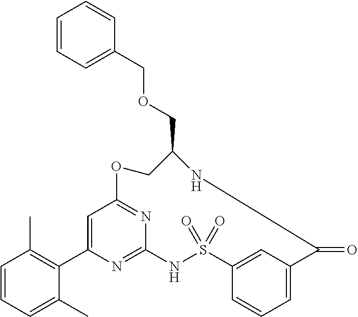 [SMILES: Cc1cccc(C)c1-c1cc2nc(n1)NS(=O)(=O)c1cccc(c1)C(=O)N[C@H](COCc1ccccc1)CO2] | 1.6 | 544.178 | 545.4 | A |
| 487 | 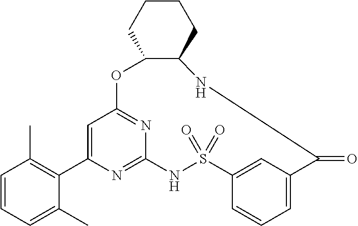 [SMILES: Cc1cccc(C)c1-c1cc2nc(n1)NS(=O)(=O)c1cccc(c1)C(=O)N[C@@H]1CCCC[C@H]1O2] | 1.48 | 478.167 | 479.3 | A |
| 488 | 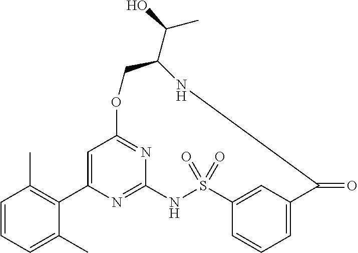 [SMILES: Cc1cccc(C)c1-c1cc2nc(n1)NS(=O)(=O)c1cccc(c1)C(=O)N[C@H]([C@H](C)O)CO2] | 1.02 | 468.147 | 469.4 | A |
| 489 | 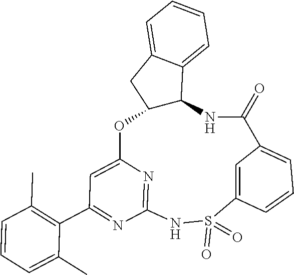 [SMILES: Cc1cccc(C)c1-c1cc2nc(n1)NS(=O)(=O)c1cccc(c1)C(=O)N[C@@H]1c3ccccc3C[C@H]1O2] | 1.42 | 512.152 | 513.3 | A |
| 490 | 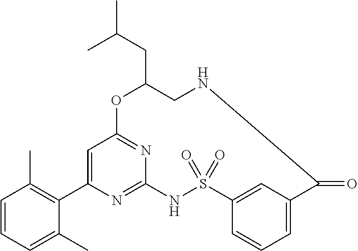 [SMILES: Cc1cccc(C)c1-c1cc2nc(n1)NS(=O)(=O)c1cccc(c1)C(=O)NCC(CC(C)C)O2] | 1.7 | 480.183 | 481.4 | A |
| 491 | 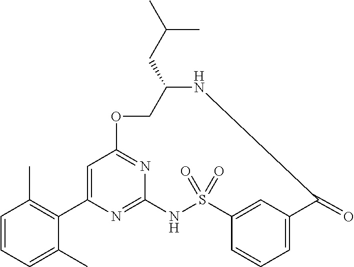 [SMILES: Cc1cccc(C)c1-c1cc2nc(n1)NS(=O)(=O)c1cccc(c1)C(=O)N[C@@H](CC(C)C)CO2] | 1.6 | 480.183 | 481.3 | A |

TABLE 5
LCMS characterization data

| | | LCMS | | | | |
|---|---|---|---|---|---|---|
| Cmpd | | Rt | Calc. | | LC | |
| Number | Structure | (min) | Mass | M + 1 | Met. | NMR |
| 496 | 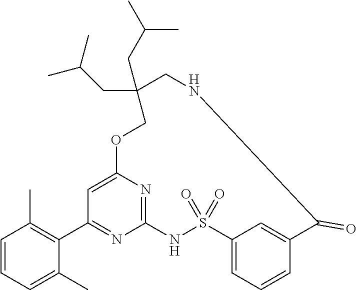 [SMILES: Cc1cccc(C)c1-c1cc2nc(n1)NS(=O)(=O)c1cccc(c1)C(=O)NCC(CC(C)C)(CC(C)C)CO2] | 2.03 | 550.261 | 551.5 | A | |
| 494 | 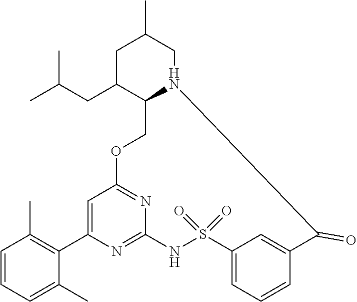 | 2.03 | 550.261 | 551.5 | A | ¹H NMR (400 MHz, CDCl₃) δ 8.76 (t, J = 1.7 Hz, 1H), 8.08 (dt, J = 7.8, 1.5 Hz, 1H), 7.85 (dt, J = 7.7, 1.3 Hz, 1H), 7.66 (t, J = 7.8 Hz, 1H), 7.22 (t, J = 7.6 Hz, 1H), 7.07 (d, J = 7.6 Hz, 2H), 6.31 (d, J = 3.4 Hz, 1H), 5.56 (d, J = 11.1 Hz, 1H), 1.68-1.65 (m, 1H), 5.26 (dd, J = 11.3, 4.3 Hz, 1H), 4.15 (t, J = 11.5 Hz, 1H), 3.85 (t, J = 11.2 Hz, 1H), 2.03 (s, 6H), 1.59 (s, 0H), 1.22-1.16 (m, 1H), 1.11 (dd, J = 10.1, 3.9 Hz, 1H), 1.07-0.98 (m, 1H), |
| | | | | | | 0.95 (d, J = 6.6 Hz, 3H), |
| | | | | | | 0.87 (d, J = 6.5 Hz, 3H), |
| | | | | | | 0.57 (d, J = 6.3 Hz, 3H), |
| | | | | | | 0.44 (d, J = 23.7 Hz, 1H), |
| | | | | | | 0.33 (d, J = 6.3 Hz, 3H). |
| 493 | 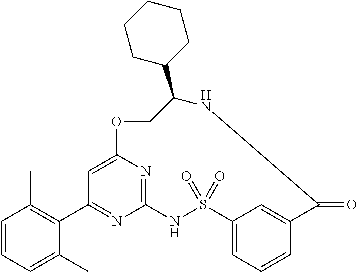 [SMILES: Cc1cccc(C)c1-c1cc2nc(n1)NS(=O)(=O)c1cccc(c1)C(=O)N[C@H](C1CCCCC1)CO2] | 1.67 | 506.199 | 507.4 | A | |
| 492 | 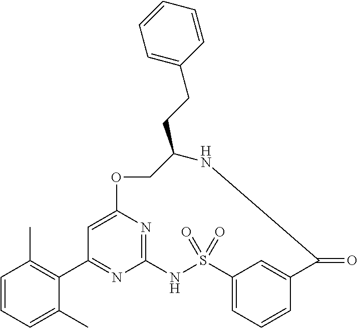 [SMILES: Cc1cccc(C)c1-c1cc2nc(n1)NS(=O)(=O)c1cccc(c1)C(=O)N[C@H](CCc1ccccc1)CO2] | 1.55 | 528.183 | 529.4 | D | |

TABLE 6
NMR characterization data

| Com- | |
|---|---|
| pound | |
| number | NMR |
| 118 | ¹H NMR (400 MHz, Methanol-d₄) δ 8.76 (s, 1H), 8.06 (dt, |
| | J = 7.3, 1.7 Hz, 1H), 7.77-7.66 (m, 2H), 7.27 (t, J = 7.6 Hz, |
| | 1H), 7.19-7.10 (m, 4H), 6.93 (d, J = 8.5 Hz, 1H), 6.37 (dd, |
| | J = 10.9, 4.0 Hz, 1H), 6.25 (s, 1H), 6.02 (s, 2H), 3.66 (dd, |
| | J = 14.0, 4.0 Hz, 1H), 3.42-3.33 (m, 2H), 2.12 (s, 6H). |
| 121 | ¹H NMR (500 MHz, DMSO-d₆) δ 8.52 (s, 1H), 7.98-7.84 |
| | (m, 2H), 7.74-7.57 (m, 2H), 7.24 (t, J = 7.7 Hz, 1H), 7.11 |
| | (d, J = 7.6 Hz, 2H), 6.40 (s, 1H), 5.10 (dd, J = 11.0, 4.0 Hz, |
| | 1H), 3.83 (t, J = 11.1 Hz, 1H), 3.29 (s, 2H), 1.98 (d, J = 51.8 |
| | Hz, 6H), 1.51 (dd, J = 14.6, 8.7 Hz, 1H), 1.42 (d, J = 14.4 |
| | Hz, 1H), 0.54 (s, 9H). |
| 122 | ¹H NMR (500 MHz, DMSO-d₆) δ 8.65 (s, 1H), 7.96 (s, 1H), |
| | 7.69 (s, 3H), 7.52 (d, J = 8.0 Hz, 2H), 7.33 (d, J = 8.1 Hz, |
| | 2H), 7.26 (s, 1H), 7.12 (d, J = 7.7 Hz, 2H), 6.37-6.15 (m, |
| | 2H), 4.50 (t, J = 5.2 Hz, 1H), 3.46 (q, J = 6.0 Hz, 3H), 2.68 |
| | (dd, J = 8.9, 6.6 Hz, 2H), 2.05 (s, 6H), 1.77 (dt, J = 14.0, 6.5 |
| | Hz, 2H). |
| 133 | ¹H NMR (400 MHz, DMSO-d₆) δ 8.66 (s, 1H), 8.05-7.84 |
| | (m, 1H), 7.81-7.57 (m, 2H), 7.48 (s, 4H), 7.42 (d, J = 9.6 |
| | Hz, 1H), 7.37-7.20 (m, 1H), 7.13 (d, J = 7.6 Hz, 2H), 6.55- |
| | 6.29 (m, 1H), 3.48-3.34 (m, 1H), 2.18-1.93 (m, 6H), 1.72- |
| | 1.52 (m, 0H), 1.32 (s, 9H), 1.27-1.10 (m, 1H), 0.64 (t, J = |
| | 7.2 Hz, 3H). |
| 163 | ¹H NMR (500 MHz, DMSO-d₆) δ 9.89 (s, 1H), 8.47 (s, 1H), |
| | 7.90 (t, J = 9.3 Hz, 2H), 7.65 (d, J = 5.5 Hz, 2H), 7.17 (t, J = |
| | 7.9 Hz, 1H), 6.75 (dd, J = 17.6, 7.9 Hz, 2H), 6.33 (s, 1H), |
| | 5.16 (dd, J = 10.9, 3.8 Hz, 1H), 3.86 (t, J = 11.0 Hz, 1H), |
| | 3.27 (d, J = 11.0 Hz, 1H), 2.05 (s, 3H), 1.60-1.48 (m, 2H), |
| | 1.21 (t, J = 12.3 Hz, 1H), 0.79 (d, J = 6.5 Hz, 3H), 0.32 (d, |
| | J = 6.4 Hz, 3H). |
| 164 | ¹H NMR (500 MHz, DMSO-d₆) δ 8.46 (s, 1H), 7.97-7.82 |
| | (m, 2H), 7.69 (d, J = 4.5 Hz, 2H), 6.36 (s, 1H), 5.12-5.02 |
| | (m, 1H), 3.77 (t, J = 11.1 Hz, 1H), 3.16 (d, J = 10.7 Hz, 1H), |
| | 1.58-1.42 (m, 2H), 1.32 (s, 3H), 1.17 (d, J = 26.9 Hz, 3H), |
| | 0.79 (d, J = 6.0 Hz, 5H), 0.28 (d, J = 6.4 Hz, 3H). |
| 165 | ¹H NMR (500 MHz, DMSO-d₆) δ 8.47 (s, 1H), 7.89 (d, J = |
| | 10.0 Hz, 2H), 7.74-7.57 (m, 2H), 7.32 (d, J = 16.9 Hz, 1H), |
| | 7.19 (d, J = 8.1 Hz, 1H), 7.09 (d, J = 8.5 Hz, 1H), 6.25 (s, |
| | 1H), 5.77 (s, 1H), 5.14 (d, J = 13.7 Hz, 1H), 4.36-3.99 (m, |
| | 1H), 3.86 (t, J = 11.6 Hz, 2H), 1.67-1.46 (m, 7H), 1.31- |
| | 1.07 (m, 4H), 0.90-0.80 (m, 1H), 0.76 (d, J = 7.4 Hz, 3H), |
| | 0.25 (d, J = 7.0 Hz, 3H). |
| 166 | ¹H NMR (500 MHz, DMSO-d₆) δ 8.49 (s, 1H), 7.90 (d, J = |
| | 10.5 Hz, 2H), 7.67 (s, 2H), 7.27 (d, J = 7.7 Hz, 1H), 7.19- |
| | 7.03 (m, 1H), 6.42-6.30 (m, 1H), 5.15 (d, J = 13.2 Hz, |
| | 1H), 3.89 (t, J = 11.6 Hz, 2H), 3.44-3.27 (m, 1H), 2.08 (s, |
| | 3H), 1.88 (s, 2H), 1.59-1.38 (m, 2H), 1.31-1.04 (m, 8H), |
| | 0.77 (dd, J = 6.9, 3.1 Hz, 3H), 0.26 (s, 3H). |
| 167 | ¹H NMR (500 MHz, DMSO-d₆) δ 8.47 (d, J = 27.4 Hz, 1H), |
| | 7.90 (d, J = 9.1 Hz, 2H), 7.68 (s, 2H), 7.31 (s, 1H), 7.17 (s, |
| | 1H), 7.12 (d, J = 7.7 Hz, 1H), 6.87 (s, 1H), 6.56 (t, J = 18.1 |
| | Hz, 1H), 6.16 (s, 1H), 6.03 (s, 1H), 5.14 (d, J = 10.8 Hz, 1H), |
| | 3.86 (q, J = 10.9 Hz, 1H), 3.79 (s, 2H), 3.70 (s, 4H), 3.55 (s, |
| | 3H), 2.11 (d, J = 22.5 Hz, 3H), 1.93 (s, 1H), 1.50 (d, J = 12.7 |
| | Hz, 2H), 1.19 (d, J = 13.1 Hz, 1H), 0.74 (s, 3H), 0.23 (s, 3H). |
| 168 | ¹H NMR (500 MHz, DMSO-d₆) δ 8.46 (s, 1H), 7.91 (d, J = |
| | 11.3 Hz, 2H), 7.72 (d, J = 26.9 Hz, 2H), 7.32 (s, 1H), 7.20 (s, |
| | 3H), 7.01 (s, 1H), 6.81 (s, 1H), 6.65 (s, 1H), 6.28 (s, 1H), |
| | 5.16 (s, 1H), 3.82 (dt, J = 27.5, 12.8 Hz, 2H), 3.25 (s, 2H), |
| | 2.09 (s, 3H), 1.93 (s, 1H), 1.53 (s, 2H), 1.24 (s, 1H), 0.79 (d, |
| | J = 30.3 Hz, 3H), 0.32 (d, J = 73.1 Hz, 3H). |
| 170 | ¹H NMR (500 MHz, DMSO-d₆) δ 8.52 (s, 1H), 7.95 (d, J = |
| | 6.9 Hz, 1H), 7.73-7.65 (m, 3H), 7.32 (d, J = 16.0 Hz, 2H), |
| | 7.17 (d, J = 7.7 Hz, 1H), 7.12 (d, J = 7.6 Hz, 1H), 6.92 (s, |
| | 1H), 6.67 (s, 2H), 6.24 (s, 1H), 5.16 (d, J = 12.3 Hz, 1H), |
| | 3.87 (t, J = 11.0 Hz, 1H), 3.71 (s, 1H), 3.32 (d, J = 10.9 Hz, |
| | 1H), 3.15 (2, 2H), 2.16 (s, 3H), 2.05 ( s, 3H), 1.52 (t, J = |
| | 11.5 Hz, 2H), 1.26 (dd, J = 26.1, 14.7 Hz, 1H), 0.77 (d, J = |
| | 6.5 Hz, 3H), 0.32 (d, J = 6.3 Hz, 3H). |
| 171 | ¹H NMR (400 MHz, Methanol-d₄) δ 8.62 (t, J = 1.8 Hz, 1H), |
| | 8.09 (dt, J = 7.5, 1.6 Hz, 1H), 7.83-7.68 (m, 2H), 7.27 (t, |
| | J = 7.7 Hz, 1H), 7.14 (d, J = 7.6 Hz, 2H), 6.31 (s, 1H), 5.49- |
| | 5.37 (m, 1H), 4.48 (t, J = 11.7 Hz, 1H), 4.37 (ddd, J = 11.4, |
| | 7.4, 4.1 Hz, 1H), 2.12 (s, 6H). |
| 213 | ¹H NMR (500 MHz, DMSO-d₆) δ 13.56-11.85 (broad d, |
| | 1H), 8.47 (s, 1H), 7.94 (s, 1H), 7.81 (d, J = 9.9 Hz, 1H), 7.65 |
| | (s, 2H), 7.33-7.23 (m, 1H), 7.20-7.07 (m, 2H), 6.43 (s, |
| | 1H), 5.21-5.12 (m, 1H), 3.88 (t, J = 11.3 Hz, 1H), 2.25- |
| | 2.12 (m, 1H), 2.11 (s, 3H), 2.09-1.95 (m, 1H), 1.68-1.58 |
| | (m, 1H), 1.49-1.40 (m, 1H), 1.38-1.10 (m, 6H), 1.00-0.75 |
| | (m, 3H), 0.70-0.59 (m, 1H), 0.59-0.50 (m, 1H), 0.31-0.15 |
| | (m, 1H) |
| 214 | ¹H NMR (500 MHz, DMSO-d₆) δ 13.50-11.81 (broad d, |
| | 1H), 8.50 (s, 1H), 7.95 (s, 1H), 7.83 (d, J = 10.0 Hz, 1H), |
| | 7.68 (s, 2H), 7.40-7.28 (m, 1H), 7.23 (d, J = 7.7 Hz, 1H), |
| | 7.19 (d, J = 7.5 Hz, 1H), 6.28 (s, 1H), 5.91 (s, 1H), 5.62 (dt, |
| | J = 16.1, 6.1 Hz, 1H), 5.16 (dd, J = 10.9, 3.9 Hz, 1H), 3.90 |
| | (t, J = 11.0 Hz, 1H), 2.13 (s, 3H), 2.13-2.00 (m, 1H), 1.96- |
| | 1.84 (m, 1H), 1.62-1.51 (m, 1H), 1.48-1.38 (m, 1H), 1.38- |
| | 1.19 (m, 2H), 1.17-1.05 (m, 1H), 1.01-0.89 (m, 1H), 0.81- |
| | 0.62 (m, 2H), 0.62-0.49 (m, 1H), 0.32-0.18 (m, 1H). |
| 215 | ¹H NMR (500 MHz, DMSO-d₆) δ 13.38-11.65 (broad d, |
| | 1H), 8.52 (s, 1H), 7.93 (s, 1H), 7.86 (d, J = 8.9 Hz, 1H), 7.68 |
| | (s, 2H), 7.56 (d, J = 7.8 Hz, 1H), 7.37 (t, J = 7.2 Hz, 1H), |
| | 7.24 (d, J = 7.5 Hz, 1H), 6.35 (s, 2H), 5.76 (d, J = 15.9 Hz, |
| | 1H), 5.68 (ddt, J = 16.8, 10.0, 6.9 Hz, 1H), 5.16 (dd, J = |
| | 10.0, 2.8 Hz, 2H), 4.91 (d, J = 13.9 Hz, 1H), 4.88 (d, J = 5.9 |
| | Hz, 1H), 3.89 (t, J = 11.1 Hz, 1H), 3.35-3.25 (m, 1H, |
| | overlaps with water peak), 2.23-1.93 (m, 3H), 1.88-1.70 |
| | (m, 2H), 1.55-1.40 (m, 2H), 1.32-1.22 (m, 1H), 1.21-1.10 |
| | (m, 2H), 1.10-0.97 (m, 3H), 0.97-0.81 (m, 2H) |
| 217 | ¹H NMR (500 MHz, DMSO-d₆) δ 13.51-11.74 (broad d, |
| | 1H), 8.49 (s, 1H), 7.96 (s, 1H), 7.89 (d, J = 10.0 Hz, 1H), |
| | 7.70 (s, 2H), 7.46-7.34 (m, 2H), 7.32 (t, J = 7.4 Hz, 1H), |
| | 7.26 (d, J = 7.1 Hz, 1H), 6.76 (s, 1H), 5.15 (dd, J = 10.9, 3.4 |
| | Hz, 1H), 3.94 (t, J = 11.2 Hz, 1H), 3.34-3.15 (m, 1H), 2.97 |
| | (td, J = 12.5, 4.9 Hz, 1H), 2.13 (td, J = 13.5, 3.1 Hz, 1H), |
| | 1.67-1.54 (m, 1H), 1.53-1.42 (m, 1H), 1.29-1.19 (m, 1H), |
| | 1.19-0.96 (m, 3H), 0.92-0.75 (m, 3H), 0.75-0.58 (m, 2H), |
| | 0.29-0.06 (m, 1H) |
| 218 | ¹H NMR (500 MHz, DMSO-d₆) δ 13.68-11.74 (broad d, |
| | 1H), 8.47 (s, 1H), 7.98 (s, 1H), 7.90 (d, J = 9.9 Hz, 1H), 7.72 |
| | (s, 2H), 7.46-7.38 (m, 2H), 7.34 (t, J = 7.4 Hz, 1H), 7.30 (d, |
| | J = 7.6 Hz, 1H), 6.37 (s, 1H), 6.11 (d, J = 15.9 Hz, 1H), 5.40- |
| | 5.26 (m, 1H), 5.11 (dd, J = 11.0, 4.0 Hz, 1H), 3.90 (t, J = |
| | 11.2 Hz, 1H), 3.25-3.02 (m, 1H), 2.16-2.01 (m, 1H, |
| | overlapping with MeCN impurity), 1.71-1.58 (m, 1H), 1.57- |
| | 1.45 (m, 2H), 1.20-1.10 (m, 2H), 1.08-0.96 (m, 2H), 0.54- |
| | 0.41 (m, 1H), 0.37-0.24 (m, 1H) |
| 219 | ¹H NMR (500 MHz, DMSO-d₆) δ 13.83-11.84 (broad d, |
| | 1H) 8.51 (s, 1H), 8.08 (d, J = 10.1 Hz, 1H), 7.99 (d, J = 7.5 |
| | Hz, 1H), 7.79-7.66 (m, 2H), 7.49 (d, J = 7.7 Hz, 1H), 7.42 |
| | (t, J = 7.5 Hz, 1H), 7.34 (t, J = 7.5 Hz, 1H), 7.23 (d, J = 7.6 |
| | Hz, 1H), 6.33 (s, 1H), 6.26 (d, J = 16.1 Hz, 1H), 5.18 (dd, |
| | J = 11.6, 3.8 Hz, 1H), 4.68-4.50 (m, 1H), 4.00 (t, J = 11.1 |
| | Hz, 1H), 3.38-3.23 (m, 1H, overlapping with water peak), |
| | 2.09-2.00 (m, 1H), 1.98-1.89 (m, 1H), 1.72 (q, J = 12.9, |
| | 12.2 Hz, 1H), 1.41-1.31 (m, 1H), 1.17-0.97 (m, 3H), 0.26- |
| | 0.11 (m, 1H). |
| 35 | ¹H NMR (500 MHz, DMSO-d₆) δ 8.52 (s, 1H), 7.95 (s, 1H), |
| | 7.89 (d, J = 9.9 Hz, 1H), 7.76-7.65 (m, 3H), 7.54-7.46 (m, |
| | 1H), 7.42-7.35 (m, 2H), 6.77 (dd, J = 17.4, 11.3 Hz, 1H), |
| | 6.42 (s, 1H), 5.79 (d, J = 17.4 Hz, 1H), 5.68 (ddt, J = 16.9, |
| | 10.2, 6.6 Hz, 1H), 5.30 (d, J = 11.0 Hz, 1H), 5.15 (dd, J = |
| | 10.6, 3.1 Hz, 1H), 4.95-4.83 (m, 2H), 3.87 (t, J = 11.2 Hz, |
| | 1H), 3.23 (q, J = 10.8 Hz, 1H), 1.87-1.77 (m, 2H), 1.59- |
| | 1.40 (m, 2H), 1.32-1.24 (m, 1H), 1.22-1.11 (m, 2H), 1.10- |
| | 1.02 (m, 2H), 1.01-0.89 (m, 3H) |
| 220 | ¹H NMR (500 MHz, DMSO-d₆) δ 8.52 (s, 1H), 7.96 (s, 1H), |
| | 7.89 (d, J = 9.7 Hz, 1H), 7.78-7.66 (m, 3H), 7.54-7.46 (m, |
| | 1H), 7.43-7.34 (m, 2H), 6.77 (dd, J = 17.5, 10.5 Hz, 1H), |
| | 6.42 (s, 1H), 5.79 (d, J = 17.3 Hz, 1H), 5.64-5.51 (m, 1H), |
| | 5.29 (d, J = 11.1 Hz, 1H), 5.15 (dd, J = 10.7, 3.0 Hz, 1H), |
| | 4.88-4.77 (m, 2H), 3.87 (t, J = 11.1 Hz, 1H), 3.24 (q, J = |
| | 10.6 Hz, 1H), 1.83 (hept, J = 7.3 Hz, 2H), 1.57-1.40 (m, |
| | 2H), 1.34-1.24 (m, 1H), 1.19-1.10 (m, 2H), 1.04-0.89 (m, 3H) |
| 221 | ¹H NMR (500 MHz, DMSO-d₆) δ 8.51 (s, 1H), 7.95 (d, J = |
| | 5.6 Hz, 1H), 7.89 (d, J = 9.8 Hz, 1H), 7.76-7.62 (m, 3H), |
| | 7.54-7.45 (m, 1H), 7.43-7.35 (m, 2H), 6.77 (dd, J = 16.0, |
| | 11.5 Hz, 1H), 6.42 (s, 1H), 5.79 (d, J = 17.4 Hz, 1H), 5.70- |
| | 5.59 (m, 1H), 5.30 (d, J = 11.1 Hz, 1H), 5.14 (dd, J = 11.0, |
| | 3.7 Hz, 1H), 4.89-4.79 (m, 2H), 3.87 (t, J = 11.1 Hz, 1H), |
| | 3.23 (q, J = 10.4 Hz, 1H), 1.84 (q, J = 7.0 Hz, 2H), 1.58- |
| | 1.49 (m, 1H), 1.49-1.40 (m, 1H), 1.35-1.26 (m, 1H), 1.14- |
| | 0.90 (m, 3H) |
| 227 | ¹H NMR (400 MHz, dimethylsulfoxide-d₆) δ 13.57-11.43 |
| | (broad d, 1H), 8.67 (s, 1H), 7.94 (d, J = 7.2 Hz, 1H), 7.69 (s, |
| | 1H), 7.67 (s, 1H), 7.50 (s, 4H), 7.44 (d, J = 9.8 Hz, 1H), 7.27 |
| | (t, J = 7.5 Hz, 1H), 7.14 (d, J = 7.6 Hz, 2H), 6.42 (s, 1H), |
| | 6.41 (s, 1H), 3.57-3.48 (m, 1H), 2.05 (s, 6H), 1.54-1.44 |
| | (m, 1H), 1.33 (s, 9H), 1.32-1.21 (m, 2H), 0.97-0.84 (m, |
| | 1H), 0.51 (t, J = 7.2 Hz, 3H) |
| 229 | ¹H NMR (400 MHz, dimethylsulfoxide-d₆) δ 13.80-10.98 |
| | (broad d, 1H, D₂O exchangeable), 8.62 (s, 1H), 7.99-7.87 |
| | (bs, 1H), 7.75-7.61 (bs, 2H), 7.53-7.46 (m, 4H), 7.41 (d, |
| | J = 9.8 Hz, 1H, D₂O exchangeable), 7.27 (t, J = 7.2 Hz, 1H), |
| | 7.13 (d, J = 7.5 Hz, 2H), 6.65-6.40 (bs, 1H), 6.35 (d, J = |
| | 4.0 Hz, 1H), 3.48-3.27 (m, 1H, overlaps with water peak), |
| | 2.17-1.97 (m, 1H), 2.03 (s, 6H), 1.88-1.76 (m, 1H), 1.74- |
| | 1.44 (m, 4H), 1.42-1.33 (m, 2H), 1.32 (s, 9H), 0.88-0.71 |
| | (m, 1H) |
| 230 | ¹H NMR (400 MHz, dimethylsulfoxide-d₆) δ 13.42-12.43 |
| | (bs, 1H, D₂O exchangeable), 8.46 (s, 1H), 7.97 (d, J = 10.9 |
| | Hz, 1H, D₂O exchangeable), 7.89 (d, J = 7.3 Hz, 1H), 7.77- |
| | 7.56 (m, 2H), 7.26 (t, J = 7.6 Hz, 1H), 7.12 (d, J = 7.6 Hz, |
| | 2H), 6.30 (s, 1H), 5.61-5.48 (m, 1H), 3.83-3.71 (m, 1H), |
| | 2.21-1.94 (m, 8H), 1.86-1.69 (m, 3H), 1.66-1.41 (m, 5H) |
| 231 | ¹H NMR (400 MHz, dimethylsulfoxide-d₆) δ 13.65-11.60 |
| | (broad d, 1H, D₂O exchangeable), 8.70 (s, 1H), 7.97 (s, 1H), |
| | 7.85-7.64 (m, 8H), 7.83-7.76 (m, 1H, D20 exchangeable), |
| | 7.54-7.47 (m, 2H), 7.44-7.37 (m, 1H), 7.26 (t, J = 7.7 Hz, |
| | 1H), 7.12 (d, J = 7.6 Hz, 2H), 6.36 (s, 1H), 6.31 (dd, J = |
| | 10.5, 3.7 Hz, 1H), 3.56 (ddd, J = 13.0, 9.1, 3.8 Hz, 2H), 3.43- |
| | 3.34 (m, 1H, overlaps with water peak), 2.06 (s, 6H) |
| 240 | ¹H NMR (300 MHz, DMSO-d₆) ppm 0.56 (d, J = 6.5 Hz, 3H), |
| | 0.65 (d, J-6.5 Hz, 3H), 1.26-1.42 (m, 2H), 1.77 (dquin, |
| | J = 13.5, 6.7 Hz, 1H), 2.00 (br. s., 6H), 2.23-2.33 (m, 1H), |
| | 3.69 (t, J = 10.6 Hz, 1H), 3.87-4.00 (m, 1H), 4.05-4.20 (m, |
| | 1H), 5.23 (dd, J = 10.4, 2.8 Hz, 1H), 6.27 (s, 1H), 7.04-7.15 |
| | (m, 2H), 7.18-7.28 (m, 1H), 7.42-7.54 (m, 2H), 7.61-7.71 (m, |
| | 1H), 8.56 (s, 1H). |
| 243 | ¹H NMR (400 MHz, DMSO-d₆) δ 8.41 (t, J = 1.5 Hz, 1H), |
| | 8.14 (dd, J = 7.9, 1.7 Hz, 2H), 8.01 (s, 2H), 7.67 (t, J = 7.8 |
| | Hz, 1H), 7.26 (d, J = 8.3 Hz, 3H), 7.10 (dd, J = 19.5, 7.8 Hz, |
| | 4H), 6.25 (s, 1H), 5.26 (d, J = 6.4 Hz, 1H), 3.25-3.00 (m, |
| | 3H), 2.91 (dd, J = 14.0, 6.3 Hz, 1H), 1.99 (s, 6H), 1.23 (s, 9H). |
| 244 | ¹H NMR (400 MHz, DMSO-d₆) δ 8.45 (s, 1H), 8.21 (s, 1H), |
| | 7.89 (s, 1H), 7.67 (s, 2H), 7.30-7.21 (m, 1H), 7.11 (d, J = |
| | 7.1 Hz, 2H), 6.25 (s, 1H), 5.05 (d, J = 10.0 Hz, 1H), 2.98 (s, |
| | 1H), 1.83-1.64 (m, 14H). Some signals overlapped with |
| | solvents. |
| 246 | ¹H NMR (400 MHz, DMSO-d₆) δ 8.64 (s, 1H), 7.91 (d, J = |
| | 4.2 Hz, 1H), 7.69 (d, J = 7.4 Hz, 3H), 7.58 (d, J = 8.1 Hz, |
| | 1H), 7.42 (d, J = 4.0 Hz, 2H), 7.32 (dt, J = 8.1, 4.2 Hz, 1H), |
| | 7.23 (t, J = 7.5 Hz, 1H), 7.09 (d, J = 7.0 Hz, 2H), 6.43 (s, |
| | 1H), 6.10 (s, 1H), 3.99 (dd, J = 7.7, 5.1 Hz, 1H), 3.38 (t, J = |
| | 6.2 Hz, 1H), 2.85 (d, J = 16.2 Hz, 1H), 2.00 (s, 6H). |
| 249 | ¹H NMR (400 MHz, DMSO-d₆) δ 8.62 (s, 1H), 8.42 (d, J = |
| | 9.6 Hz, 1H), 7.97 (s, 1H), 7.69 (s, 2H), 7.24 (t, J = 7.7 Hz, |
| | 1H), 7.08 (dd, J = 19.9, 7.8 Hz, 4H), 6.79 (d, J = 8.7 Hz, |
| | 2H), 6.17 (s, 1H), 5.33 (dd, J = 10.9, 3.7 Hz, 1H), 4.60-4.43 |
| | (m, 2H), 4.34 (t, J = 11.2 Hz, 1H), 2.01 (s, 6H), 1.21 (d, J = |
| | 6.0 Hz, 6H). |
| 250 | ¹H NMR (400 MHz, DMSO-d₆) δ 8.71 (s, 1H), 7.98 (d, J = |
| | 8.4 Hz, 3H), 7.86 (d, J = 8.3 Hz, 2H), 7.79-7.60 (m, 3H), |
| | 7.25 (t, J = 7.3 Hz, 1H), 7.12 (d, J = 7.5 Hz, 2H), 6.44-6.21 |
| | (m, 2H), 3.61-3.50 (m, 1H), 3.28-3.15 (m, 1H), 2.04 (s, 6H). |
| 251 | ¹H NMR (400 MHz, DMSO-d₆) δ 13.43 (s, 1H), 12.71 (s, |
| | 1H), 12.54-12.40 (m, 5H), 12.32 (d, J = 8.5 Hz, 2H), 12.01 |
| | (t, J = 7.7 Hz, 1H), 11.87 (d, J = 7.6 Hz, 2H), 11.10 (s, 1H), |
| | 11.00 (dd, J = 10.8, 4.5 Hz, 1H), 8.35-8.23 (m, 1H), 8.06- |
| | 7.95 (m, 1H), 6.80 (s, 6H). |
| 32 | ¹H NMR (400 MHz, DMSO-d₆) δ 8.70 (s, 1H), 7.96 (s, 1H), |
| | 7.84-7.57 (m, 5H), 7.52 (d, J = 6.4 Hz, 2H), 7.25 (t, J = 7.1 |
| | Hz, 1H), 7.11 (d, J = 7.5 Hz, 2H), 6.45-6.17 (m, 2H), 3.60- |
| | 3.45 (m, 1H), 2.05 (s, 6H). One aliphatic proton overlapped |
| | with water. |
| 252 | ¹H NMR (400 MHz, DMSO-d₆) δ 8.74 (s, 1H), 8.23 (s, 1H), |
| | 8.02-7.88 (m, 3H), 7.79 (dd, J = 9.6, 5.3 Hz, 1H), 7.70 (t, |
| | J = 7.8 Hz, 3H), 7.25 (t, J = 7.3 Hz, 1H), 7.12 (d, J = 7.6 Hz, |
| | 2H), 6.50-6.21 (m, 2H), 3.62-3.48 (m, 1H), 2.04 (s, 6H). |
| | One aliphatic proton overlapped with water. |
| 31 | ¹H NMR (400 MHz, DMSO-d₆) δ 8.71 (s, 1H), 8.03-7.88 |
| | (m, 2H), 7.81-7.64 (m, 3H), 7.61 (d, J = 9.3 Hz, 1H), 7.55- |
| | 7.46 (m, 2H), 7.25 (t, J = 7.3 Hz, 1H), 7.11 (d, J = 7.6 Hz, |
| | 2H), 7.01 (dd, J = 10.5, 4.3 Hz, 1H), 6.34 (s, 1H), 3.52-3.37 |
| | (m, 2H), 2.04 (s, 6H). |
| 253 | ¹H NMR (400 MHz, DMSO-d₆) δ 8.74 (s, 1H), 8.22 (s, 1H), |
| | 8.10-8.02 (m, 2H), 7.99 (dd, J = 6.1, 3.4 Hz, 2H), 7.83- |
| | 7.64 (m, 4H), 7.63-7.55 (m, 2H), 7.25 (t, J = 7.3 Hz, 1H), |
| | 7.12 (d, J = 7.6 Hz, 2H), 6.50-6.24 (m, 2H), 3.61 (ddd, J = |
| | 13.6, 9.7, 3.8 Hz, 1H), 3.48-3.38 (m, 1H), 2.05 (s, 6H). |
| 18 | ¹H NMR (400 MHz, DMSO-d₆) δ 8.67 (s, 1H), 7.96 (s, 1H), |
| | 7.81-7.65 (m, 5H), 7.60 (d, J = 8.4 Hz, 2H), 7.25 (t, J = 6.0 |
| | Hz, 1H), 7.11 (d, J = 7.5 Hz, 2H), 6.46-6.12 (m, 2H), 3.60- |
| | 3.44 (m, 1H), 3.30-3.18 (m, 1H), 2.04 (s, 6H). |
| 256 | ¹H NMR (400 MHz, Chloroform-d) δ 8.86 (t, J = 1.8 Hz, |
| | 1H), 8.00-7.80 (m, 2H), 7.64 (dt, J = 7.8, 3.7 Hz, 3H), 7.61- |
| | 7.56 (m, 2H), 7.31-7.20 (m, 1H), 7.08 (d, J = 7.6 Hz, 2H), |
| | 6.35-6.29 (m, 1H), 6.28 (s, 1H), 5.44 (dd, J = 10.9, 4.8 Hz, |
| | 1H), 4.01 (ddd, J = 13.7, 10.9, 4.2 Hz, 1H), 3.31 (ddd, J = |
| | 13.8, 10.7, 4.7 Hz, 1H), 2.07 (s, 6H), 1.63 (s, 6H). |
| 258 | ¹H NMR (400 MHz, Chloroform-d) δ 8.90 (t, J = 1.8 Hz, |
| | 1H), 8.10 (d, J = 8.0 Hz, 1H), 7.93 (dt, J = 7.7, 1.3 Hz, 1H), |
| | 7.70 (t, J = 7.8 Hz, 1H), 7.51 (d, J = 8.4 Hz, 2H), 7.44 (d, |
| | J = 8.5 Hz, 2H), 7.23 (d, J = 7.6 Hz, 1H), 7.09 (d, J = 7.6 Hz, |
| | 2H), 6.41-6.20 (m, 2H), 5.39 (dd, J = 11.0, 4.7 Hz, 1H), |
| | 4.00 (ddd, J = 14.7, 11.0, 4.1 Hz, 1H), 3.35 (ddd, J = 14.9, |
| | 10.9, 4.7 Hz, 1H), 2.09(s, 6H), 1.72 (q, J = 7.3 Hz, 6H), 0.69 |
| | (t, J = 7.4 Hz, 9H). |
| 260 | ¹H NMR (400 MHz, Chloroform-d) δ 8.90 (s, 1H), 8.10 (d, |
| | J = 7.9 Hz, 1H), 7.91 (d, J = 7.8 Hz, 1H), 7.69 (t, J = 7.7 Hz, |
| | 1H), 7.58 (d, J = 7.1 Hz, 1H), 7.47 (d, J = 8.2 Hz, 2H), 7.20 |
| | (dd, J = 7.8, 5.5 Hz, 2H), 7.08 (d, J = 7.6 Hz, 2H), 6.37- |
| | 6.17 (m, 2H), 5.4 (m, 1H), 4.05 (m, 1H), 3.40 (m, 1H), 2.06 |
| | (s, 2H), 1.99-1.91 (m, 1H), 1.27 (s, 1H). |
| 262 | ¹H NMR (400 MHz, Chloroform-d) δ 8.86 (t, J = 1.8 Hz, |
| | 1H), 8.00-7.80 (m, 2H), 7.64 (dt, J = 7.8, 3.7 Hz, 3H), 7.61- |
| | 7.56 (m, 2H), 7.31-7.20 (m, 1H), 7.08 (d, J = 7.6 Hz, 2H), |
| | 6.35-6.29 (m, 1H), 6.28 (s, 1H), 5.44 (dd, J = 10.9, 4.8 Hz, |
| | 1H), 4.01 (ddd, J = 13.7, 10.9, 4.2 Hz, 1H), 3.31 (ddd, J = |
| | 13.8, 10.7, 4.7 Hz, 1H), 2.07 (s, 6H), 1.63 (s, 6H). |
| 263 | ¹H NMR (400 MHz, Chloroform-d) δ 8.88 (s, 1H), 8.19 (d, |
| | J = 7.7 Hz, 1H), 7.95 (d, J = 7.6 Hz, 1H), 7.74 (t, J = 7.9 Hz, |
| | 1H), 7.49 (d, J = 7.9 Hz, 2H), 7.28 (d, J = 7.9 Hz, 2H), 7.24 |
| | (d, J = 7.7 Hz, 1H), 7.10 (d, J = 7.7 Hz, 2H), 6.36 (s, 1H), |
| | 6.31 (d, J = 7.6 Hz, 1H), 5.40 (s, 1H), 3.99 (s, 1H), 3.38 (s, |
| | 1H), 2.57 (s, 2H), 2.12 (s, 6H), 0.95 (s, 9H). |
| 266 | ¹H NMR (400 MHz, Chloroform-d) δ 8.90 (t, J = 1.8 Hz, |
| | 1H), 8.10 (d, J = 8.0 Hz, 1H), 7.93 (dt, J = 7.7, 1.3 Hz, 1H), |
| | 7.70 (t, J = 7.8 Hz, 1H), 7.51 (d, J = 8.4 Hz, 2H), 7.44 (d, |
| | J = 8.5 Hz, 2H), 7.23 (d, J = 7.6 Hz, 1H), 7.09 (d, J = 7.6 Hz, |
| | 2H), 6.41-6.20 (m, 2H), 5.39 (dd, J = 11.0, 4.7 Hz, 1H), |
| | 4.00 (ddd, J = 14.7, 11.0, 4.1 Hz, 1H), 3.35 (ddd, J = 14.9, |
| | 10.9, 4.7 Hz, 1H), 2.09 (s, 6H), 1.72 (q, J = 7.3 Hz, 6H), 0.69 |
| | (t, J = 7.4 Hz, 9H). |
| 268 | ¹H NMR (400 MHz, Chloroform-d) δ 8.90 (s, 1H), 8.10 (d, |
| | J = 7.9 Hz, 1H), 7.91 (d, J = 7.8 Hz, 1H), 7.69 (t, J = 7.7 Hz, |
| | 1H), 7.58 (d, J = 7.1 Hz, 1H), 7.47 (d, J = 8.2 Hz, 2H), 7.20 |
| | (dd, J = 7.8, 5.5 Hz, 2H), 7.08 (d, J = 7.6 Hz, 2H), 6.37- |
| | 6.17 (m, 2H), 5.4 (m, 1H), 4.05 (m, 1H), 3.40 (m, 1H), 2.06 |
| | (s, 2H), 1.99-1.91 (m, 1H), 1.27 (s, 1H). |
| 274 | ¹H NMR (400 MHz, Chloroform-d) δ 8.98-8.80 (m, 1H), |
| | 7.98-7.79 (m, 2H), 7.63 (t, J = 7.8 Hz, 1H), 7.58-7.48 (m, |
| | 2H), 7.47-7.37 (m, 2H), 7.29-7.19 (m, 1H), 7.07 (d, J = |
| | 7.6 Hz, 2H), 6.26 (q, J = 4.1, 3.1 Hz, 2H), 5.44 (dd, J = 11.0, |
| | 4.5 Hz, 1H), 4.03 (t, J = 13.1 Hz, 1H), 3.52-3.23 (m, 1H), |
| | 2.06 (d, J = 1.4 Hz, 6H), 1.38 (d, J = 1.5 Hz, 9H). |
| 278 | ¹H NMR (400 MHz, Chloroform-d) δ 9.06 (s, 1H), 8.14 (d, |
| | J = 7.9 Hz, 1H), 7.96 (d, J = 7.8 Hz, 1H), 7.71 (t, J = 7.8 Hz, |
| | 1H), 7.68-7.63 (m, 1H), 7.61-7.56 (m, 1H), 7.47-7.38 (m, |
| | 2H), 7.35 (dd, J = 10.7, 4.6 Hz, 1H), 7.23-7.17 (m, 1H), |
| | 7.07 (d, J = 7.6 Hz, 2H), 6.26 (s, 1H), 5.43 (d, J = 7.2 Hz, |
| | 1H), 4.30-4.08 (m, 1H), 3.84-3.61 (m, 1H), 2.06 (s, 6H), |
| | 1.62 (s, 9H). |
| 280 | ¹H NMR (400 MHz, Chloroform-d) δ 8.97 (t, J = 1.7 Hz, |
| | 1H), 8.03 (d, J = 7.9 Hz, 1H), 7.87 (dt, J = 7.7, 1.3 Hz, 1H), |
| | 7.66 (t, J = 7.8 Hz, 1H), 7.54 (dd, J = 7.6, 1.7 Hz, 1H), 7.43 |
| | (ddd, J = 8.3, 7.4, 1.7 Hz, 1H), 7.25-7.19 (m, 1H), 7.13- |
| | 7.04 (m, 4H), 7.02 (dd, J = 8.5, 1.0 Hz, 1H), 6.27 (s, 1H), |
| | 5.48 (dd, J = 10.9, 4.8 Hz, 1H), 3.95 (s, 3H), 3.92-3.76 (m, |
| | 1H), 3.37 (ddd, J = 13.9, 11.0, 4.8 Hz, 1H), 2.07 (s, 6H). |
| 279 | ¹H NMR (400 MHz, Chloroform-d) δ 8.91 (t, J = 1.9 Hz, |
| | 1H), 8.01 (d, J = 8.0 Hz, 1H), 7.90 (d, J = 7.7 Hz, 1H), 7.67 |
| | (t, J = 7.8 Hz, 1H), 7.62-7.51 (m, 1H), 7.42-7.30 (m, 3H), |
| | 7.23 (t, J = 7.6 Hz, 1H), 7.08 (d, J = 7.6 Hz, 2H), 6.81 (dd, |
| | J = 10.9, 4.5 Hz, 1H), 6.29 (s, 1H), 5.44 (dd, J = 10.8, 4.8 Hz, |
| | 1H), 3.96 (ddd, J = 14.6, 10.7, 4.4 Hz, 1H), 3.42 (ddd, J = |
| | 14.5, 10.8, 4.9 Hz, 1H), 2.62 (s, 3H), 1.56 (s, 6H). |
| 290 | ¹H NMR (400 MHz, DMSO-d₆) δ 11.56 (s, 1H), 9.49 (s, 1H), |
| | 9.10 (s, 1H), 7.95 (s, 2H), 7.64 (s, 1H), 7.46 (s, 2H), 7.35 (t, |
| | J = 7.5 Hz, 2H), 7.26 (s, 2H), 7.10 (d, J = 7.6 Hz, 2H), 6.54 (s, |
| | 1H), 5.05 (s, 1H), 4.83 (s, 1H), 4.42 (s, 1H), 2.29 (s, 2H), |
| | 1.97 (s, 6H). |
| 291 | ¹H NMR (400 MHz, DMSO-d₆) δ 11.84 (s, 1H), 8.79 (d, J = |
| | 9.6 Hz, 1H), 7.85 (d, J = 7.7 Hz, 1H), 7.81 (d, J = 7.7 Hz, |
| | 1H), 7.72 (dd, J = 7.4, 1.9 Hz, 1H), 7.67 (t, J = 7.5 Hz, 1H), |
| | 7.51-7.39 (m, 3H), 7.24 (t, J = 7.6 Hz, 1H), 7.13 (t, J = 8.7 |
| | Hz, 3H), 6.78 (q, J = 8.4 Hz, 1H), 4.55 (s, 1H), 2.03 (s, 6H), |
| | 1.44 (d, J = 6.7 Hz, 3H). |
| 292 | ¹H NMR (400 MHz, DMSO-d₆) ä 11.83 (s, 1H), 8.79 (d, J = |
| | 9.6 Hz, 1H), 7.85 (d, J = 7.9 Hz, 1H), 7.81 (d, J = 7.6 Hz, |
| | 1H), 7.72 (dd, J = 7.4, 1.9 Hz, 1H), 7.67 (t, J = 8.0 Hz, 1H), |
| | 7.51-7.39 (m, 3H), 7.24 (t, J = 7.6 Hz, 1H), 7.13 (t, J = 8.8 |
| | Hz, 3H), 6.78 (s, 1H), 4.55 (s, 1H), 2.03 (s, 6H), 1.44 (d, J = |
| | 6.7 Hz, 3H). |
| 293 | ¹H NMR (400 MHz, DMSO-d₆) δ 11.61 (s, 1H), 9.23 (s, 1H), |
| | 8.47 (s, 1H), 7.99 (s, 1H), 7.88 (s, 1H), 7.67 (s, 1H), 7.42 (s, |
| | 1H), 7.34 (t, J = 7.5 Hz, 1H), 7.29-7.21 (m, 2H), 7.17 (s, |
| | 1H), 7.04 (s, 2H), 6.52 (s, 1H), 5.54 (s, 2H), 4.52 (d, J = 6.2 |
| | Hz, 2H), 1.73 (s, 6H). |
| 294 | ¹H NMR (400 MHz, DMSO-d₆) δ 13.05 (s, 1H), 9.39 (s, 1H), |
| | 9.15 (s, 1H), 8.00 (s, 2H), 7.67 (s, 1H), 7.61 (s, 1H), 7.49- |
| | 7.38 (m, 1H), 7.38-7.30 (m, 2H), 7.29-7.21 (m, 1H), 7.14 |
| | (d, J = 7.6 Hz, 2H), 6.63 (s, 1H), 5.74 (s, 2H), 4.67 (s, 2H), |
| | 2.22-1.95 (m, 6H). |
| 296 | ¹H NMR (400 MHz, DMSO-d₆) δ 12.90 (s, 1H), 8.21 (s, 1H), |
| | 7.87 (d, J = 10.4 Hz, 1H), 7.82 (d, J = 7.9 Hz, 1H), 7.63 (t, |
| | J = 7.7 Hz, 1H), 7.51 (d, J = 7.5 Hz, 1H), 7.21 (t, J = 7.6 Hz, |
| | 1H), 7.10 (d, J = 7.7 Hz, 1H), 7.05 (d, J = 7.6 Hz, 1H), 6.84 |
| | (s, 1H), 6.26 (s, 1H), 4.91 (dd, J = 12.0, 3.2 Hz, 1H), 3.91 (t, |
| | J = 11.7 Hz, 1H), 3.11-2.92 (m, 1H), 2.04 (s, 3H), 1.79 (s, |
| | 3H), 1.38 (t, J = 11.5 Hz, 2H), 1.12 (d, J = 9.5 Hz, 1H), 0.71 |
| | (d, J = 6.5 Hz, 3H), 0.03 (d, J = 6.4 Hz, 3H). |
| 298 | ¹H NMR (400 MHz, DMSO-d₆) δ 13.53-11.66 (broad m, |
| | 1H), 8.55 (s, 1H), 8.07-7.82 (m, 2H), 7.68 (br s, 2H), 7.50- |
| | 7.35 (m, 1H), 7.24 (dd, J = 18.6, 7.7 Hz, 2H), 6.46 (br s, 1H), |
| | 5.10 (dd, J = 11.0, 4.0 Hz, 1H), 3.86 (t, J = 11.1 Hz, 1H), |
| | 3.38 (overlapped with water, br s, 1H), 2.61-2.53 |
| | (overlapped with DMSO, m, 1H), 2.15 (p, J = 6.9 Hz, 1H), |
| | 1.52 (dd, J = 14.6, 8.6 Hz, 1H), 1.40 (d, J = 14.4 Hz, 1H), |
| | 1.18 (d, J = 6.8 Hz, 3H), 1.09 (d, J = 6.8 Hz, 3H), 1.01 (br s, |
| | 3H), 0.95 (d, J = 6.8 Hz, 3H), 0.54 (s, 9H). |
| 70 | ¹H NMR (500 MHz, DMSO-d₆) δ 13.24 (broad s, 1H), 9.24 |
| | (s, 1H), 9.05 (s, 1H), 8.36 (d, J = 9.6 Hz, 1H), 7.29 (t, J = 7.6 |
| | Hz, 1H), 7.15 (d, J = 7.7 Hz, 2H), 6.40 (s, 1H), 5.69 (dd, J = |
| | 9.8, 4.2 Hz, 1H), 3.75 (t, J = 10.4 Hz, 1H), 3.34-3.23 (m, |
| | 1H overlapped with water), 2.16 (br s, 3H), 2.03 (br s, 3H), |
| | 1.70-1.57 (m, 1H), 1.51 (ddd, J = 14.3, 10.6, 3.9 Hz, 1H), |
| | 1.28 (ddd, J = 13.3, 9.9, 2.8 Hz, 1H), 0.81 (d, J = 6.7 Hz, |
| | 3H), 0.45 (d, J = 6.5 Hz, 3H). |
| 305 | ¹H NMR (400 MHz, DMSO-d₆) δ 8.54 (s, 1H), 7.97 (d, J = |
| | 7.4 Hz, 1H), 7.90 (d, J = 9.7 Hz, 1H), 7.71 (d, J = 9.2 Hz, |
| | 2H), 7.39 (t, J = 7.3 Hz, 1H), 7.36-7.25 (m, 3H), 6.53 (s, |
| | 1H), 5.11 (dd, J = 10.9, 4.0 Hz, 1H), 3.83 (t, J = 11.1 Hz, |
| | 1H), 2.24 (s, 3H), 1.60-1.41 (m, 2H), 0.58 (s, 9H). |
| 316 | ¹H NMR (400 MHz, DMSO-d₆) δ 8.48 (s, 1H), 7.93 (s, 1H), |
| | 7.86 (d, J = 9.9 Hz, 1H), 7.67 (s, 2H), 7.46-7.21 (m, 4H), |
| | 6.50 (s, 1H), 5.13 (dd, J = 10.9, 3.7 Hz, 1H), 3.85 (t, J = 11.0 |
| | Hz, 1H), 2.27 (s, 3H), 1.58 (d, J = 13.1 Hz, 1H), 1.54-1.34 |
| | (m, 4H), 1.30 (t, J = 10.8 Hz, 1H), 1.25-1.08 (m, 2H), 1.01 |
| | (d, J = 17.7 Hz, 2H), 0.90-0.75 (m, 2H), 0.32 (d, J = 12.0 |
| | Hz, 1H). |
| 358 | ¹H NMR (400 MHz, DMSO-d₆) δ 12.95 (s, 1H), 8.22 (d, J = |
| | 7.8 Hz, 1H), 7.43 (s, 1H), 7.26 (d, J = 8.1 Hz, 1H), 7.13 (d, |
| | J = 7.6 Hz, 2H), 6.31 (s, 1H), 4.96 (dd, J = 10.7, 4.0 Hz, 1H), |
| | 3.86 (s, 3H), 3.62 (t, J = 11.1 Hz, 1H), 2.79 (d, J = 8.5 Hz, |
| | 1H), 1.99 (s, 6H), 1.58 (dd, J = 14.7, 8.9 Hz, 1H), 1.43 (d, |
| | J = 14.5 Hz, 1H), 0.70 (s, 9H). |
| 77 | ¹H NMR (400 MHz, DMSO-d₆) δ 13.28 (s, 1H), 7.83 (d, J = |
| | 8.2 Hz, 1H), 7.29 (t, J = 7.6 Hz, 1H), 7.19 (s, 1H), 7.15 (d, |
| | J = 7.6 Hz, 2H), 6.38 (s, 1H), 4.75 (dd, J = 10.5, 4.5 Hz, 1H), |
| | 3.93 (s, 3H), 3.54 (t, J = 11.0 Hz, 1H), 2.75 (d, J = 7.9 Hz, |
| | 1H), 2.05 (d, J = 47.2 Hz, 6H), 1.49 (dd, J = 14.6, 8.9 Hz, |
| | 1H), 1.34 (d, J = 14.3 Hz, 1H), 0.73 (s, 9H). |
| 365 | ¹H NMR (400 MHz, DMSO-d₆) δ 13.03 (s, 1H), 8.47 (s, |
| | 1H), 7.89 (dd, J = 29.2, 8.4 Hz, 2H), 7.78-7.54 (m, 2H), |
| | 7.26 (t, J = 7.6 Hz, 1H), 7.12 (d, J = 7.6 Hz, 2H), 6.40 (s, |
| | 1H), 5.13 (dd, J = 10.9, 3.9 Hz, 1H), 3.87 (t, J = 11.0 Hz, |
| | 1H), 3.37 (d, J = 10.9 Hz, 1H), 2.01 (s, 6H), 1.37 (dddd, J = |
| | 80.5, 61.2, 41.2, 13.5 Hz, 8H), 0.97 (q, J = 11.0, 10.0 Hz, |
| | 2H), 0.80 (q, J = 12.5, 11.9 Hz, 2H), 0.28 (t, J = 11.4 Hz, 1H). |
| 387 | ¹H NMR (499 MHz, DMSO-d₆) δ 13.11 (s, 1H), 8.48 (s, 1H), |
| | 7.89 (d, J = 10.3 Hz, 2H), 7.66 (s, 2H), 7.28 (s, 1H), 7.19- |
| | 6.87 (m, 2H), 6.45 (d, J = 73.3 Hz, 1H), 5.18 (d, J = 10.6 Hz, |
| | 1H), 3.87 (t, J = 10.9 Hz, 1H), 3.26 (s, 1H), 2.26 (d, J = 31.0 |
| | Hz, 1H), 2.12 (s, 2H), 1.98 (s, 1H), 1.90 (s, 1H), 1.52 (d, J = |
| | 19.7 Hz, 5H), 1.42 (s, 1H), 1.33 (d, J = 12.2 Hz, 1H), 1.14 |
| | (d, J = 12.1 Hz, 3H), 0.95 (d, J = 8.3 Hz, 2H), 0.79 (d, J = |
| | 6.6 Hz, 4H), 0.62 (d, J = 11.1 Hz, 1H), 0.29 (s, 3H). |
| 389 | ¹H NMR (500 MHz, DMSO-d₆) δ 13.14 (s, 1H), 8.47 (s, 1H), |
| | 7.89 (d, J = 9.7 Hz, 2H), 7.65 (s, 2H), 7.27 (s, 1H), 7.12 (s, |
| | 2H), 6.39 (s, 1H), 5.18 (s, 1H), 3.95-3.78 (m, 1H), 3.17 (s, |
| | 1H), 2.28 (d, J = 86.2 Hz, 2H), 2.09 (d, J = 20.7 Hz, 2H), |
| | 1.90 (s, 1H), 1.53 (s, 2H), 1.25 (d, J = 76.2 Hz, 4H), 0.79 (d, |
| | J = 6.7 Hz, 5H), 0.51 (dd, J = 20.7, 6.2 Hz, 4H), 0.27 (d, J = |
| | 26.8 Hz, 3H). |
| 390 | ¹H NMR (500 MHz, DMSO-d₆) δ 12.50 (s, 1H), 8.52 (s, 1H), |
| | 7.93 (d, J = 5.9 Hz, 1H), 7.67 (d, J = 5.2 Hz, 3H), 7.43-7.18 |
| | (m, 2H), 7.12 (dd, J = 41.7, 7.7 Hz, 1H), 6.26 (d, J = 32.6 |
| | Hz, 1H), 6.05-5.88 (m, 1H), 5.16 (ddd, J = 15.7, 10.7, 3.8 |
| | Hz, 1H), 3.88 (td, J = 11.0, 3.7 Hz, 1H), 3.30 (d, J = 10.3 |
| | Hz, 1H), 3.13 (s, 3H), 2.10-2.04 (m, 2H), 1.54 (dd, J = 12.9, |
| | 9.0 Hz, 2H), 1.24 (t, J = 11.7 Hz, 1H), 0.82 (d, J = 24.1 Hz, |
| | 6H), 0.77 (t, J = 6.5 Hz, 3H), 0.32 (t, J = 7.5 Hz, 3H). |
| 392 | ¹H NMR (500 MHz, DMSO-d₆) δ 12.01 (s, 1H), 8.48 (s, 1H), |
| | 8.05-7.93 (m, 1H), 7.89 (d, J = 9.8 Hz, 1H), 7.82 (s, 1H), |
| | 7.70 (d, J = 4.9 Hz, 2H), 7.51 (t, J = 7.9 Hz, 1H), 7.32 (dt, |
| | J = 16.5, 7.7 Hz, 3H), 7.09 (t, J = 7.4 Hz, 1H), 6.96 (dd, J = |
| | 18.5, 8.1 Hz, 3H), 6.77 (s, 1H), 5.09 (dd, J = 11.2, 3.9 Hz, |
| | 1H), 3.80 (t, J = 11.1 Hz, 1H), 3.17 (q, J = 10.9 Hz, 1H), |
| | 1.57-1.36 (m, 2H), 1.11 (t, J = 12.3 Hz, 1H), 0.74 (d, J = |
| | 6.5 Hz, 3H), 0.19 (d, J = 6.4 Hz, 3H). |
| 394 | ¹H NMR (500 MHz, DMSO-d₆) δ 12.33 (s, 1H), 8.53 (s, 1H), |
| | 7.95 (d, J = 7.2 Hz, 1H), 7.69 (t, J = 10.5 Hz, 3H), 7.29 (t, |
| | J = 7.8 Hz, 1H), 7.16 (d, J = 7.6 Hz, 1H), 7.10 (d, J = 7.7 Hz, |
| | 1H), 6.90 (s, 2H), 6.73 (s, 2H), 6.24 (s, 1H), 5.16 (dd, J = |
| | 10.6, 3.9 Hz, 1H), 3.87 (t, J = 11.0 Hz, 1H), 3.71 (d, J = 14.3 |
| | Hz, 1H), 3.32 (d, J = 10.5 Hz, 1H), 2.23 (s, 3H), 2.05 (d, J = |
| | 13.7 Hz, 3H), 1.53 (t, J = 11.1 Hz, 2H), 1.25 (t, J = 12.2 Hz, |
| | 1H), 0.78 (d, J = 6.5 Hz, 3H), 0.33 (d, J = 6.4 Hz, 3H). |
| 396 | ¹H NMR (500 MHz, DMSO-d₆) δ 13.02 (s, 1H), 8.65 (s, 1H), |
| | 7.95 (s, 1H), 7.75-7.59 (m, 3H), 7.54 (d, J = 7.8 Hz, 2H), |
| | 7.32 (d, J = 7.7 Hz, 2H), 7.24 (t, J = 7.7 Hz, 1H), 7.10 (d, |
| | J = 7.6 Hz, 2H), 6.29 (s, 1H), 6.21 (dd, J = 10.7, 4.4 Hz, 1H), |
| | 4.11 (s, 1H), 3.51 (ddd, J = 13.8, 9.2, 4.0 Hz, 1H), 2.56 (s, |
| | 1H), 2.08 (s, 6H), 2.03 (s, 6H). |
| 398 | ¹H NMR (400 MHz, DMSO-d₆) δ 13.02 (s, 1H), 8.64 (s, 1H), |
| | 7.95 (s, 1H), 7.68 (s, 3H), 7.57-7.49 (m, 2H), 7.37-7.30 |
| | (m, 2H), 7.24 (d, J = 8.0 Hz, 1H), 7.11 (d, J = 7.6 Hz, 2H), |
| | 6.30 (s, 1H), 6.21 (d, J = 10.1 Hz, 1H), 3.51 (t, J = 10.1 Hz, |
| | 1H), 2.58 (s, 1H), 2.54 (s, 1H), 2.09 (s, 6H), 2.04 (s, 6H). |
| 399 | ¹H NMR (400 MHz, DMSO-d₆) δ 13.07 (s, 1H), 8.69 (s, 1H), |
| | 7.96 (s, 1H), 7.79-7.63 (m, 7H), 7.24 (d, J = 7.2 Hz, 1H), |
| | 7.11 (d, J = 7.6 Hz, 2H), 6.47-6.14 (m, 2H), 3.55 (ddd, J = |
| | 13.5, 6.6, 3.0 Hz, 1H), 3.28 (d, J = 3.8 Hz, 1H), 2.10-1.95 |
| | (m, 9H). |
| 34 | ¹H NMR (400 MHz, DMSO-d₆) δ 12.94 (s, 1H), 8.50 (s, 1H), |
| | 8.33 (dd, J = 9.5, 5.3 Hz, 1H), 7.92 (d, J = 7.1 Hz, 1H), 7.75- |
| | 7.59 (m, 2H), 7.26 (t, J = 7.6 Hz, 1H), 7.12 (d, J = 7.7 Hz, |
| | 2H), 6.33 (s, 1H), 5.31-5.12 (m, 1H), 4.00-3.88 (m, 2H), |
| | 3.40-3.34 (m, 2H), 3.20-3.04 (m, 2H), 2.31-2.21 (m, 1H), |
| | 2.13-1.97 (m, 6H), 1.77 (d, J = 12.3 Hz, 1H), 1.65-1.46 |
| | (m, 3H). |
| 401 | ¹H NMR (400 MHz, DMSO-d₆) δ 13.07 (s, 1H), 8.71 (s, 1H), |
| | 7.97 (s, 1H), 7.87 (d, J = 1.3 Hz, 4H), 7.77 (d, J = 8.5 Hz, |
| | 1H), 7.69 (s, 2H), 7.24 (d, J = 7.9 Hz, 1H), 7.12 (d, J = 7.6 |
| | Hz, 2H), 6.33 (d, J = 12.9 Hz, 2H), 3.58 (t, J = 10.8 Hz, 1H), |
| | 3.31-3.22 (m, 1H), 2.06 (d, J = 10.6 Hz, 6H). |
| 402 | ¹H NMR (400 MHz, DMSO-d₆) & 12.90 (s, 1H), 8.71 (s, 1H), |
| | 8.00-7.93 (m, 1H), 7.87 (s, 4H), 7.76 (d, J = 8.4 Hz, 1H), |
| | 7.67 (d, J = 5.3 Hz, 2H), 7.28-7.19 (m, 1H), 7.11 (d, J = 7.6 |
| | Hz, 2H), 6.33 (dd, J = 10.8, 4.2 Hz, 2H), 3.58 (qd, J = 8.2, |
| | 6.9, 4.6 Hz, 1H), 3.30-3.22 (m, 1H), 2.04 (s, 6H), 1.23 (s, |
| | 1H) with minimal residual solvent remaining (hexanes). |
| 403 | ¹H NMR (400 MHz, DMSO-d₆) δ 13.07 (s, 1H), 8.71 (s, 1H), |
| | 7.97 (s, 1H), 7.87 (d, J = 1.7 Hz, 4H), 7.78 (dd, J = 9.5, 5.3 |
| | Hz, 1H), 7.69 (s, 2H), 7.25 (t, J = 7.6 Hz, 1H), 7.12 (d, J = |
| | 7.6 Hz, 2H), 6.33 (dd, J = 10.9, 4.1 Hz, 2H), 3.65-3.48 (m, |
| | 1H), 3.27 (ddd, J = 13.9, 10.8, 5.2 Hz, 1H), 2.06 (d, J = 10.3 |
| | Hz, 6H). |
| 404 | ¹H NMR (400 MHz, DMSO-d₆) δ 13.03 (s, 1H), 8.65 (s, 1H), |
| | 7.96 (s, 1H), 7.68 (dd, J = 9.4, 5.3 Hz, 3H), 7.49-7.34 (m, |
| | 3H), 7.33-7.19 (m, 2H), 7.11 (d, J = 7.7 Hz, 2H), 6.32 (s, |
| | 1H), 6.23 (dd, J = 11.0, 4.1 Hz, 1H), 3.60-3.45 (m, 1H), |
| | 3.44-3.34 (m, 1H), 2.39 (s, 3H), 2.06 (d, J = 10.4 Hz, 6H). |
| 407 | ¹H NMR (400 MHz, DMSO-d₆) δ 13.03 (s, 1H), 8.64 (s, 1H), |
| | 7.95 (s, 1H), 7.65 (d, J = 16.1 Hz, 3H), 7.55-7.44 (m, 2H), |
| | 7.31 (d, J = 7.9 Hz, 2H), 7.25 (t, J = 7.6 Hz, 1H), 7.11 (d, |
| | J = 7.6 Hz, 2H), 6.45-6.25 (m, 1H), 6.25-6.15 (m, 1H), 3.50 |
| | (ddt, J = 13.6, 9.8, 4.1 Hz, 1H), 3.36 (d, J = 5.6 Hz, 1H), |
| | 2.37 (s, 3H), 2.06 (d, J = 13.1 Hz, 6H). |
| 30 | ¹H NMR (400 MHz, DMSO-d₆) δ 13.04 (s, 1H), 8.80 (t, J = |
| | 5.3 Hz, 1H), 8.66 (s, 1H), 7.99-7.87 (m, 1H), 7.84-7.73 (m, |
| | 1H), 7.66 (s, 2H), 7.26 (t, J = 7.6 Hz, 1H), 7.12 (d, J = 7.6 |
| | Hz, 2H), 6.32 (s, 1H), 5.62 (dd, J = 9.3, 4.7 Hz, 1H), 3.21 |
| | (dtt, J = 13.3, 8.1, 4.0 Hz, 4H), 2.04 (s, 6H), 1.52-1.39 (m, |
| | 2H), 0.94 (s, 9H). |
| 410 | ¹H NMR (400 MHz, DMSO-d₆) δ 10.21-10.06 (m, 1H), |
| | 8.62-8.51 (m, 1H), 8.19 (dd, J = 9.9, 5.2 Hz, 1H), 7.95 (dt, |
| | J = 6.7, 2.0 Hz, 1H), 7.70 (d, J = 7.2 Hz, 2H), 7.27 (t, J = |
| | 7.6 Hz, 1H), 7.13 (d, J = 7.6 Hz, 2H), 6.36 (s, 1H), 5.76 (d, |
| | J = 9.4 Hz, 1H), 3.80 (dd, J = 13.6, 4.0 Hz, 1H), 3.49 (dd, J = |
| | 14.0, 5.0 Hz, 3H), 3.38-3.32 (m, 1H), 3.18-3.10 (m, 1H), |
| | 3.07-2.99 (m, 2H), 2.13-2.02 (m, 6H), 1.81 (q, J = 11.8, |
| | 8.9 Hz, 4H), 1.70 (dd, J = 13.4, 4.0 Hz, 1H), 1.48-1.37 (m, 1H). |
| 412 | ¹H NMR (400 MHz, DMSO-d₆) δ 13.18 (s, 1H), 9.04 (s, 1H), |
| | 8.81 (d, J = 23.0 Hz, 2H), 8.10 (d, J = 9.8 Hz, 1H), 7.29 (t, |
| | J = 7.6 Hz, 1H), 7.14 (d, J = 7.6 Hz, 2H), 6.41 (s, 1H), 5.16 (d, |
| | J = 14.7 Hz, 1H), 3.89 (t, J = 11.2 Hz, 1H), 3.24 (d, J = 8.5 |
| | Hz, 1H), 2.07 (s, 4H), 1.46 (d, J = 23.3 Hz, 5H), 1.25 (d, J = |
| | 22.1 Hz, 3H), 0.98 (s, 2H), 0.83 (t, J = 11.4 Hz, 2H), 0.35 (d, |
| | J = 8.6 Hz, 1H). |
| 413 | ¹H NMR (400 MHz, DMSO-d₆) δ 13.16 (s, 1H), 9.03 (s, 1H), |
| | 8.83 (d, J = 21.9 Hz, 2H), 8.11 (d, J = 9.7 Hz, 1H), 7.28 (s, |
| | 1H), 7.14 (d, J = 7.4 Hz, 2H), 6.41 (s, 1H), 5.18 (d, J = 10.8 |
| | Hz, 1H), 3.89 (s, 1H), 3.21 (s, 1H), 2.09 (d, J = 66.4 Hz, 7H), |
| | 1.56 (s, 2H), 1.22 (s, 1H), 0.78 (d, J = 6.5 Hz, 3H), 0.33 (d, |
| | J = 6.3 Hz, 3H). |
| 414 | ¹H NMR (400 MHz, DMSO-d₆) δ 9.45 (s, 1H), 8.33 (s, 1H), |
| | 7.94 (d, J = 7.6 Hz, 2H), 7.65 (t, J = 7.7 Hz, 1H), 7.30-7.20 |
| | (m, 1H), 7.11 (d, J = 7.6 Hz, 2H), 6.64 (s, 1H), 4.62 (d, J = |
| | 91.0 Hz, 2H), 3.77 (s, 3H), 3.58 (s, 2H), 3.41 (d, J = 27.4 Hz, |
| | 2H), 3.21 (s, 1H), 2.02 (s, 6H), 1.77-1.56 (m, 4H), 1.51- |
| | 1.22 (m, 8H). |
| 418 | ¹H NMR (400 MHz, DMSO-d₆) δ 9.86 (s, 1H), 9.42 (s, 1H), |
| | 8.60 (s, 1H), 7.94 (s, 5H), 7.68 (d, J = 7.8 Hz, 1H), 7.25 (t, |
| | J = 7.6 Hz, 1H), 7.12 (d, J = 7.7 Hz, 2H), 4.62 (s, 1H), 3.33 (s, |
| | 3H), 3.17 (s, 4H), 2.89 (s, 2H), 2.04 (s, 9H), 1.63 (s, 1H). |
| 419 | ¹H NMR (400 MHz, DMSO-d₆) δ 9.46 (s, 1H), 8.35 (s, 1H), |
| | 8.10 (s, 3H), 7.96 (s, 2H), 7.67 (s, 1H), 7.25 (s, 1H), 6.60 (s, |
| | 1H), 4.67 (s, 1H), 4.36 (s, 1H), 3.72 (d, J = 178.1 Hz, 6H), |
| | 2.04 (s, 6H), 1.62 (d, J = 91.9 Hz, 7H), 0.97-0.81 (m, 6H). |
| 420 | ¹H NMR (400 MHz, DMSO-d₆) δ 9.46 (s, 1H), 8.37 (s, 1H), |
| | 8.10 (s, 3H), 7.96 (s, 2H), 7.67 (s, 1H), 7.25 (s, 1H), 7.13 (d, |
| | J = 7.6 Hz, 2H), 6.59 (s, 1H), 4.62 (s, 1H), 4.34 (s, 1H), 3.96- |
| | 3.51 (m, 5H), 2.04 (s, 6H), 1.70-1.22 (m, 9H), 0.96-0.76 |
| | (m, 3H). |
| 422 | ¹H NMR (400 MHz, DMSO-d₆) δ 9.52 (d, J = 55.8 Hz, 2H), |
| | 8.98 (s, 1H), 8.36 (s, 1H), 7.94 (s, 2H), 7.66 (s, 1H), 7.25 (s, |
| | 1H), 7.12 (d, J = 7.4 Hz, 2H), 6.62 (s, 1H), 4.57 (d, J = 9.1 |
| | Hz, 2H), 3.51 (s, 5H), 3.17-2.94 (m, 2H), 2.83 (d, J = 14.9 |
| | Hz, 1H), 2.74 (d, J = 8.6 Hz, 1H), 2.05 (s, 6H), 1.69-1.41 |
| | (m, 4H). |
| 423 | ¹H NMR (400 MHz, DMSO-d₆) δ 9.43 (d, J = 45.5 Hz, 2H), |
| | 8.81 (d, J = 11.2 Hz, 1H), 8.40 (s, 1H), 7.96 (s, 2H), 7.68 (d, |
| | J = 7.8 Hz, 1H), 7.25 (t, J = 7.7 Hz, 1H), 7.13 (d, J = 7.6 Hz, |
| | 2H), 6.63 (s, 1H), 4.64 (d, J = 113.7 Hz, 1H), 3.95-3.52 (m, |
| | 4H), 3.19 (s, 1H), 3.08 (s, 1H), 2.22 (s, 2H), 2.04 (s, 9H), |
| | 1.55 (d, J = 18.9 Hz, 7H). |
| 424 | ¹H NMR (400 MHz, DMSO-d₆) δ 9.40 (s, 1H), 8.35 (s, 4H), |
| | 7.94 (d, J = 6.8 Hz, 2H), 7.66 (d, J = 7.6 Hz, 1H), 7.43-6.99 |
| | (m, 9H), 6.54 (s, 1H), 4.55 (d, J = 56.6 Hz, 3H), 3.28 (d, J = |
| | 11.1 Hz, 3H), 3.01 (d, J = 34.8 Hz, 3H), 2.02 (s, 6H), 1.37 |
| | (d, J = 51.7 Hz, 3H), 0.91 (s, 1H). |
| 425 | ¹H NMR (400 MHz, DMSO-d₆) δ 9.42 (s, 1H), 8.31 (s, 1H), |
| | 7.94 (s, 2H), 7.66 (s, 1H), 7.25 (s, 1H), 7.11 (s, 2H), 6.61 (s, |
| | 1H), 6.30 (s, 1H), 4.58 (s, 2H), 3.47 (s, 5H), 3.23 (s, 2H), |
| | 2.86 (d, J = 6.1 Hz, 2H), 2.03 (s, 6H), 1.47 (s, 2H), 1.34 (s, |
| | 2H), 0.79 (s, 10H). |
| 426 | ¹H NMR (400 MHz, DMSO-d₆) δ 9.35 (s, 1H), 8.19 (s, 1H), |
| | 7.88 (s, 2H), 7.58 (s, 1H), 7.17 (s, 1H), 7.05 (d, J = 7.6 Hz, |
| | 2H), 6.35 (s, 2H), 4.53 (s, 2H), 3.13 (s, 2H), 2.96 (d, J = 7.1 |
| | Hz, 2H), 1.95 (s, 6H), 1.52-1.20 (m, 4H), 0.91 (t, J = 7.1 |
| | Hz, 3H). |
| 427 | ¹H NMR (400 MHz, DMSO-d₆) δ 9.35 (s, 1H), 8.21 (s, 1H), |
| | 7.87 (s, 2H), 7.58 (s, 1H), 7.24-7.13 (m, 5H), 7.12-7.02 |
| | (m, 3H), 6.95 (t, J = 5.9 Hz, 1H), 6.58 (s, 1H), 4.50 (s, 2H), |
| | 4.15 (d, J = 5.8 Hz, 2H), 4.02 (d, J = 5.0 Hz, 1H), 3.42 (d, |
| | J = 17.1 Hz, 4H), 3.10 (d, J = 4.0 Hz, 3H), 1.95 (s, 6H), 1.35 |
| | (d, J = 48.3 Hz, 4H). |
| 428 | ¹H NMR (400 MHz, DMSO-d₆) δ 9.43 (s, 1H), 8.27 (s, 1H), |
| | 7.94 (s, 2H), 7.66 (t, J = 7.7 Hz, 1H), 7.25 (s, 1H), 7.12 (d, |
| | J = 7.6 Hz, 2H), 6.55 (d, J = 66.7 Hz, 2H), 4.58 (s, 2H), 3.32 |
| | (s, 2H), 3.22 (s, 5H), 3.17 (s, 2H), 2.03 (s, 6H), 1.41 (d, J = |
| | 43.2 Hz, 4H). |
| 429 | ¹H NMR (400 MHz, DMSO-d₆) δ 9.45 (s, 1H), 8.42 (s, 1H), |
| | 8.32 (s, 1H), 7.95 (s, 2H), 7.68 (s, 1H), 7.51-7.38 (m, 2H), |
| | 7.33-7.18 (m, 3H), 7.11 (d, J = 7.4 Hz, 2H), 6.92 (t, J = 7.3 |
| | Hz, 1H), 6.64 (s, 1H), 4.61 (s, 1H), 3.66 (s, 2H), 3.51 (s, 2H), |
| | 2.02 (s, 6H), 1.54 (d, J = 9.8 Hz, 2H), 1.44 (s, 2H), 1.25 (s, |
| | 1H), 0.86 (s, 1H). |
| 434 | ¹H NMR (400 MHz, DMSO-d₆) δ 9.42 (s, 1H), 8.22 (s, 1H), |
| | 7.91 (s, 2H), 7.62 (t, J = 5.6 Hz, 1H), 7.28-7.19 (m, 1H), |
| | 7.11 (d, J = 7.5 Hz, 2H), 6.56 (s, 1H), 4.51 (s, 1H), 3.43 (s, |
| | 2H), 3.15 (s, 1H), 2.86 (s, 1H), 2.40 (s, 3H), 2.02 (s, 6H), |
| | 1.89 (s, 1H), 1.61 (s, 2H), 1.38 (s, 11H). |
| 435 | ¹H NMR (400 MHz, DMSO-d₆) δ 10.39 (d, J = 21.7 Hz, |
| | 2H), 9.41 (s, 1H), 8.63 (s, 1H), 7.96 (d, J = 7.4 Hz, 2H), 7.67 |
| | (t, J = 8.1 Hz, 1H), 7.26 (t, J = 7.5 Hz, 1H), 7.13 (d, J = 7.6 |
| | Hz, 2H), 6.55 (d, J = 75.6 Hz, 1H), 4.64 (s, 1H), 3.39 (s, 5H), |
| | 3.11 (d, J = 58.0 Hz, 5H), 2.87 (d, J = 12.3 Hz, 2H), 2.70 (d, |
| | J = 4.7 Hz, 3H), 2.05 (d, J = 12.7 Hz, 12H), 1.55 (s, 3H). |
| 436 | ¹H NMR (400 MHz, DMSO-d₆) δ 10.07 (d, J = 49.7 Hz, |
| | 1H), 9.40 (s, 1H), 8.61 (s, 1H), 7.95 (s, 2H), 7.66 (q, J = 7.8 |
| | Hz, 1H), 7.31-7.22 (m, 1H), 7.13 (d, J = 7.6 Hz, 2H), 6.56 |
| | (d, J = 78.7 Hz, 1H), 4.66 (s, 1H), 3.59 (s, 2H), 3.24 (s, 2H), |
| | 3.00 (s, 3H), 2.05 (d, J = 14.6 Hz, 6H), 1.96 (s, 3H), 1.61 (s, |
| | 1H), 1.11 (s, 1H), 0.63 (s, 2H), 0.37 (s, 2H). |
| 437 | ¹H NMR (400 MHz, DMSO-d₆) δ 10.05 (d, J = 33.8 Hz, |
| | 1H), 9.41 (s, 1H), 8.60 (s, 1H), 8.14-8.05 (m, 1H), 7.90 (d, |
| | J = 45.2 Hz, 3H), 7.64 (s, 1H), 7.53-7.45 (m, 1H), 7.26 (s, |
| | 1H), 7.23 (s, 1H), 7.14 (d, J = 14.6 Hz, 3H), 7.03 (s, 1H), |
| | 4.43 (s, 4H), 3.89 (s, 3H), 3.83 (s, 2H), 3.60 (s, 2H), 3.18 (s, |
| | 2H), 2.07 (d, J = 12.2 Hz, 6H), 1.72 (d, J = 74.0 Hz, 4H). |
| 441 | ¹H NMR (400 MHz, DMSO-d₆) δ 10.07 (s, 1H), 9.41 (s, 1H), |
| | 8.62 (s, 1H), 7.95 (s, 2H), 7.67 (s, 1H), 7.26 (s, 1H), 7.13 (d, |
| | J = 7.5 Hz, 2H), 6.55 (d, J = 80.2 Hz, 1H), 4.63 (s, 2H), 3.59 |
| | (s, 1H), 3.19 (s, 1H), 3.06 (s, 2H), 1.99 (d, J = 61.6 Hz, 8H), |
| | 1.59 (d, J = 57.3 Hz, 4H), 1.22 (s, 1H), 1.10-1.04 (m, 1H), |
| | 0.88 (s, 12H). |
| 443 | ¹H NMR (400 MHz, DMSO-d₆) δ 10.00 (s, 1H), 9.42 (s, 1H), |
| | 8.68-8.53 (m, 1H), 7.97 (d, J = 13.4 Hz, 2H), 7.67 (s, 1H), |
| | 7.26 (s, 1H), 7.13 (d, J = 7.6 Hz, 2H), 6.55 (d, J = 65.7 Hz, |
| | 1H), 4.64 (s, 2H), 3.69-3.56 (m, 2H), 3.23 (s, 2H), 3.07 (s, |
| | 3H), 2.05 (d, J = 14.5 Hz, 6H), 1.89 (s, 3H), 1.58 (s, 4H), |
| | 0.99 (s, 2H), 0.91 (d, J = 5.7 Hz, 9H). |
| 444 | ¹H NMR (400 MHz, DMSO-d₆) δ 9.41 (s, 1H), 7.93 (s, 2H), |
| | 7.67 (s, 1H), 7.30 (dd, J = 26.7, 7.3 Hz, 6H), 7.13 (d, J = 7.2 |
| | Hz, 2H), 6.51 (s, 1H), 4.68 (s, 1H), 3.59 (s, 2H), 3.06 (s, 2H), |
| | 2.03 (d, J = 17.8 Hz, 9H), 1.65 (s, 2H). |
| 446 | ¹H NMR (400 MHz, DMSO-d₆) δ 9.94 (s, 1H), 9.42 (s, 1H), |
| | 8.63 (s, 1H), 7.95 (s, 2H), 7.67 (s, 1H), 7.26 (s, 1H), 7.13 (d, |
| | J = 7.6 Hz, 2H), 6.45 (s, 1H), 4.64 (s, 1H), 3.58 (s, 2H), 3.32 |
| | (s, 4H), 3.13 (s, 5H), 2.05 (d, J = 12.9 Hz, 6H), 1.92 (s, 3H), |
| | 1.60 (d, J = 13.9 Hz, 1H), 1.23 (t, J = 7.2 Hz, 3H). |
| 447 | ¹H NMR (400 MHz, DMSO-d₆) δ 14.45 (s, 1H), 9.43 (s, 1H), |
| | 8.31 (s, 1H), 7.91 (s, 3H), 7.67 (s, 1H), 7.54 (d, J = 26.1 Hz, |
| | 1H), 7.25 (s, 1H), 7.12 (d, J = 7.5 Hz, 2H), 6.57 (s, 1H), 4.77 |
| | (s, 1H), 3.72-3.32 (m, 5H), 3.17 (s, 1H), 2.91 (s, 1H), 2.71 |
| | (s, 3H), 2.03 (s, 7H), 1.43 (s, 9H). |
| 453 | ¹H NMR (400 MHz, DMSO-d₆) δ 9.46 (s, 1H), 8.31 (d, J = |
| | 5.5 Hz, 1H), 7.93 (s, 2H), 7.77 (s, 1H), 7.72 (d, J = 8.0 Hz, |
| | 1H), 7.64 (d, J = 8.3 Hz, 3H), 7.34-7.19 (m, 3H), 7.12 (d, |
| | J = 7.7 Hz, 2H), 6.66 (s, 1H), 5.57 (s, 2H), 4.66 (s, 2H), 3.84 |
| | (s, 2H), 2.02 (s, 6H), 1.60 (s, 2H), 1.49 (s, 2H). |
| 454 | ¹H NMR (400 MHz, DMSO-d₆) δ 9.43 (s, 1H), 9.05 (d, J = |
| | 8.7 Hz, 1H), 8.55 (d, J = 5.8 Hz, 1H), 8.29 (s, 1H), 7.93 (s, |
| | 2H), 7.64 (t, J = 7.7 Hz, 1H), 7.26 (s, 1H), 7.13 (d, J = 7.6 |
| | Hz, 2H), 7.06 (dd, J = 7.0, 4.1 Hz, 1H), 6.68 (s, 1H), 4.60 (s, |
| | 2H), 3.93 (s, 1H), 2.42 (s, 3H), 2.02 (s, 6H), 1.73-1.30 (m, 4H). |
| 456 | ¹H NMR (400 MHz, DMSO-d₆) δ 9.76 (s, 1H), 9.46 (s, 1H), |
| | 8.38 (d, J = 25.9 Hz, 1H), 7.95 (s, 2H), 7.67 (s, 1H), 7.47 (d, |
| | J = 25.4 Hz, 2H), 7.31 (dt, J = 31.9, 8.6 Hz, 3H), 7.16-7.05 |
| | (m, 2H), 6.64 (s, 1H), 4.56 (s, 1H), 4.27 (d, J = 63.2 Hz, 1H), |
| | 4.00 (s, 1H), 3.54 (s, 2H), 2.92 (s, 1H), 2.03 (s, 7H), 1.87 (d, |
| | J = 11.4 Hz, 1H), 1.77-1.43 (m, 10H). |
| 465 | ¹H NMR (400 MHz, DMSO-d₆) δ 9.48 (s, 1H), 8.43 (s, 1H), |
| | 7.95 (s, 2H), 7.69 (s, 1H), 7.20 (t, J = 28.0 Hz, 8H), 6.65 (s, |
| | 1H), 4.61 (s, 1H), 3.80 (d, J = 57.4 Hz, 4H), 2.46 (s, 1H), |
| | 2.34 (s, 1H), 2.04 (s, 6H), 1.44 (s, 3H), 1.09 (d, J = 25.6 Hz, |
| | 3H), 0.86 (d, J = 23.8 Hz, 3H). |
| 466 | ¹H NMR (400 MHz, DMSO-d₆) δ 11.42 (d, J = 25.8 Hz, |
| | 1H), 9.45 (s, 1H), 8.32 (s, 1H), 7.94 (s, 2H), 7.61 (s, 3H), |
| | 7.47 (s, 3H), 7.25 (t, J = 7.5 Hz, 1H), 7.12 (d, J = 7.6 Hz, |
| | 2H), 6.63 (s, 1H), 4.70 (s, 1H), 4.39 (s, 1H), 3.96 (s, 2H), |
| | 3.53 (s, 1H), 3.21 (s, 4H), 2.03 (s, 6H), 1.47 (s, 4H). |
| 468 | ¹H NMR (400 MHz, DMSO-d₆) δ 9.47 (d, J = 30.1 Hz, 1H), |
| | 8.34 (s, 1H), 7.96 (s, 2H), 7.66 (s, 1H), 7.25 (s, 1H), 7.13 (s, |
| | 2H), 6.58 (s, 1H), 4.15 (d, J = 15.2 Hz, 1H), 3.99 (d, J = 12.7 |
| | Hz, 1H), 3.55 (s, 1H), 3.44 (s, 1H), 3.10 (s, 1H), 2.22-1.84 |
| | (m, 8H), 1.49 (d, J = 63.6 Hz, 7H), 1.14 (d, J = 11.5 Hz, |
| | 1H), 1.01-0.66 (m, 12H). |
| 474 | ¹H NMR (400 MHz, DMSO-d₆) δ 9.43 (s, 1H), 8.32 (s, 1H), |
| | 7.93 (s, 2H), 7.64 (s, 1H), 7.42 (s, 3H), 7.38-7.33 (m, 2H), |
| | 7.25 (s, 1H), 7.13 (d, J = 7.5 Hz, 2H), 6.67 (s, 1H), 4.59 (s, |
| | 2H), 3.94 (s, 1H), 3.49 (s, 3H), 2.03 (s, 7H), 1.67-1.28 (m, 4H). |
| 476 | ¹H NMR (400 MHz, DMSO-d₆) δ 9.45 (s, 1H), 8.33 (s, 1H), |
| | 7.95 (s, 2H), 7.66 (s, 1H), 7.24 (d, J = 15.1 Hz, 1H), 7.11 (s, |
| | 2H), 6.64 (s, 1H), 4.61 (d, J = 93.3 Hz, 2H), 3.62 (s, 3H), |
| | 3.32 (s, 6H), 2.03 (s, 6H), 1.82 (s, 3H), 1.55 (s, 3H), 1.37 (s, |
| | 8H), 1.15 (s, 3H). |
| 477 | ¹H NMR (400 MHz, DMSO-d₆) δ 9.42 (s, 1H), 8.30 (s, 1H), |
| | 7.95 (s, 2H), 7.64 (s, 1H), 7.33-7.07 (m, 10H), 6.64 (s, 1H), |
| | 4.53 (s, 2H), 3.69 (s, 2H), 3.57 (s, 3H), 2.03 (s, 6H), 1.44 (d, |
| | J = 41.1 Hz, 5H). |
| 478 | ¹H NMR (400 MHz, DMSO-d₆) δ 10.27 (s, 1H), 9.46 (s, 1H), |
| | 8.33 (d, J = 29.6 Hz, 1H), 7.96 (s, 2H), 7.66 (s, 1H), 7.25 (s, |
| | 1H), 7.12 (d, J = 7.5 Hz, 2H), 6.61 (s, 1H), 4.67 (d, J = 75.4 |
| | Hz, 1H), 3.61 (s, 3H), 2.88 (s, 3H), 2.74 (s, 3H), 2.04 (s, 6H), |
| | 1.91-1.70 (m, 3H), 1.46 (d, J = 17.7 Hz, 5H). |
| 480 | ¹H NMR (400 MHz, DMSO-d₆) δ 9.70 (s, 1H), 9.45 (s, 1H), |
| | 8.31 (s, 1H), 7.94 (s, 2H), 7.66 (s, 1H), 7.25 (s, 1H), 7.12 (d, |
| | J = 7.6 Hz, 2H), 6.59 (s, 1H), 4.59 (s, 2H), 3.77 (s, 1H), 2.83 |
| | (s, 2H), 2.76 (d, J = 4.9 Hz, 6H), 2.04 (s, 7H), 1.51 (t, J = |
| | 24.2 Hz, 4H). |
| 481 | ¹H NMR (400 MHz, DMSO-d₆) δ 9.44 (s, 1H), 8.33 (s, 1H), |
| | 7.95 (s, 2H), 7.66 (s, 1H), 7.24 (s, 1H), 7.12 (d, J = 7.6 Hz, |
| | 2H), 6.67 (s, 1H), 4.61 (d, J = 74.7 Hz, 1H), 3.79 (s, 1H), |
| | 3.61 (s, 2H), 2.19 (s, 2H), 2.03 (s, 6H), 1.52-1.34 (m, 5H), |
| | 0.96 (s, 9H). |
| 482 | ¹H NMR (400 MHz, DMSO-d₆) δ 9.44 (s, 1H), 8.33 (s, 1H), |
| | 7.94 (s, 2H), 7.66 (s, 1H), 7.25 (s, 1H), 7.12 (d, J = 7.6 Hz, |
| | 2H), 6.62 (s, 1H), 4.60 (d, J = 64.4 Hz, 2H), 3.82 (s, 1H), |
| | 3.62 (d, J = 8.7 Hz, 3H), 2.39 (d, J = 8.2 Hz, 2H), 2.03 (s, |
| | 6H), 1.46 (d, J = 32.5 Hz, 7H), 1.08 (s, 3H), 0.83 (s, 3H). |
| 484 | ¹H NMR (400 MHz, DMSO-d₆) δ 12.92 (s, 1H), 8.48 (s, 1H), |
| | 8.10 (d, J = 9.9 Hz, 1H), 7.96-7.84 (m, 1H), 7.74-7.58 (m, |
| | 2H), 7.25 (t, J = 8.0 Hz, 1H), 7.12 (d, J = 7.6 Hz, 2H), 6.26 |
| | (s, 1H), 5.62-5.50 (m, 1H), 3.65-3.49 (m, 1H), 2.27-1.89 |
| | (m, 6H), 1.40 (d, J = 6.5 Hz, 3H), 1.01 (d, J = 6.7 Hz, 3H). |
| 488 | ¹H NMR (400 MHz, DMSO-d₆) δ 8.57-8.50 (m, 1H), 7.97- |
| | 7.88 (m, 1H), 7.73-7.62 (m, 2H), 7.30-7.21 (m, 1H), 7.16- |
| | 7.06 (m, 3H), 6.34 (s, 1H), 5.03 (dd, J = 10.7, 3.8 Hz, 1H), |
| | 4.28 (t, J = 11.1 Hz, 1H), 3.72 (tt, J = 7.1, 3.4 Hz, 1H), 3.32- |
| | 3.19 (m, 1H), 2.15-2.02 (m, 6H), 0.91 (d, J = 6.3 Hz, 3H). |

### VI. Bioactivity Data

A. 3t3 Assay

1. Membrane Potential Optical Methods for Assaying F508del Modulation Properties of Compounds

The assay utilizes fluorescent voltage sensing dyes to measure changes in membrane potential using a fluorescent plate reader (e.g., FLIPR III, Molecular Devices, Inc.) as a readout for increase in functional F508del in NIH 3T3 cells. The driving force for the response is the creation of a chloride ion gradient in conjunction with channel activation by a single liquid addition step after the cells have previously been treated with compounds and subsequently loaded with a voltage sensing dye.

2. Identification of Corrector Compounds

To identify correctors of F508del, a single-addition HTS assay format was developed. This HTS assay utilizes fluorescent voltage sensing dyes to measure changes in membrane potential on the FLIPR III as a measurement for increase in gating (conductance) of F508del in F508del NIH 3T3 cells. The F508del NIH 3T3 cell cultures were incubated with the corrector compounds at a range of concentrations for 18-24 hours at 37° C., and subsequently loaded with a redistribution dye. The driving force for the response is a Cl⁻ ion gradient in conjunction with channel activation with forskolin in a single liquid addition step using a fluorescent plate reader such as FLIPR III. The efficacy and potency of the putative F508del correctors was compared to that of the known corrector, lumacaftor, in combination with acutely added 300 nM Ivacaftor.

3. Solutions

Bath Solution #1: (in mM) NaCl 160, KCl 4.5, CaCl₂) 2, MgCl₂ 1, HEPES 10, pH 7.4 with NaOH.

Chloride-free bath solution: Chloride salts in Bath Solution #1 (above) are substituted with gluconate salts.

4. Cell Culture

NIH3T3 mouse fibroblasts stably expressing F508del are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1×NEAA, b-ME, 1×pen/strep, and 25 mM HEPES in 175 cm² culture flasks. For all optical assays, the cells were seeded at ˜20,000/well in 384-well matrigel-coated plates. For the correction assays, the cells are cultured at 37° C. with and without compounds for 16-24 hours.

B. Enteroid Assay

1. Solutions

Base medium (ADF+++) consisted of Advanced DMEM/Ham's F12, 2 mM Glutamax, 10 mM HEPES, 1 μg/mL penicillin/streptomycin.

Intestinal enteroid maintenance medium (IEMM) consisted of ADF+++, 1× B27 supplement, 1× N₂ supplement, 1.25 mM N-acetyl cysteine, 10 mM Nicotinamide, 50 ng/mL hEGF, 10 nM Gastrin, 1 μg/mL hR-spondin-1, 100 ng/mL hNoggin, TGF-b type 1 inhibitor A-83-01, 100 μg/mL Primocin, 10 μM P38 MAPK inhibitor SB202190.

Bath 1 Buffer consisted of 1 mM MgCl₂, 160 mM NaCl, 4.5 mM KCl, 10 mM HEPES, 10 mM Glucose, 2 mM CaCl₂).

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

Bath 1 Dye Solution consisted of Bath 1 Buffer, 0.04% Pluronic F127, 20 μM Methyl Oxonol, 30 μM CaCCinh-A01, 30 μM Chicago Sky Blue.

Chloride Free Dye Solution consisted of Chloride Free Buffer, 0.04% Pluronic F127, 20 μM Methyl Oxonol, 30 μM CaCCinh-A01, 30 μM Chicago Sky Blue.

Chloride Free Dye Stimulation Solution consisted of Chloride Free Dye Solution, 10 μM forskolin, 100 μM IBMX, and 300 nM Compound III.

2. Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July; 19(7):939-45).

3. Enteroid Cell Harvesting and Seeding

Cells were recovered in cell recovery solution, collected by centrifugation at 650 rpm for 5 min at 4° C., resuspended in TryPLE and incubated for 5 min at 37° C. Cells were then collected by centrifugation at 650 rpm for 5 min at 4° C. and resuspended in IEMM containing 10 μM ROCK inhibitor (RI). The cell suspension was passed through a 40 μm cell strainer and resuspended at 1×106 cells/mL in IEMM containing 10 μM RI. Cells were seeded at 5000 cells/well into multi-well plates and incubated for overnight at 37° C., 95% humidity and 5% CO₂ prior to assay.

4. Membrane Potential Dye, Enteroid Assay A

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

5. Membrane Potential Dye, Enteroid Assay B

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

C. HBE assay

1. Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (Isc) current, F508del/MF-HBE grown on Costar® Snapwell™ cell culture inserts were mounted in an Ussing chamber and the transepithelial Isc was measured under voltage-clamp recording conditions (V_{hold}=0 mV) at 37° C. The basolateral solution contained (in mM) 145 NaCl, 0.83 K₂HPO₄, 3.3 KH₂P0₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated Isc (ρA/cm²) for each condition was determined from the peak forskolin response to the steady-state current following inhibition.

2. Identification of Corrector Compounds

The activity of the CFTR corrector compounds on the CFTR-mediated Isc was determined in Ussing chamber studies as described above. The F508del/MF-HBE cell cultures were either incubated with the corrector compounds at a range of concentrations in combination with 1 μM Ivacaftor or were incubated with the corrector compounds at a single fixed concentration of 10 μM in combination with 1 μM Ivacaftor for 18-24 hours at 37° C. and in the presence of 20% human serum. The concentration of corrector compounds with 1 μM Ivacaftor during the 18-24 hours incubations was kept constant throughout the Ussing chamber measurement of the CFTR-mediated Isc to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 μM Tezacaftor and 1 μM Ivacaftor.

D. Biological Activity Tables

The following table represents CFTR modulating activity for representative compounds of the disclosure generated using one or more of the assays disclosed herein (EC₅₀:+++ is <1 μM; ++ is 1−<3 μM; + is 3−<30 μM; and ND is “not detected in this assay.” % Activity: +++ is >600%; ++ is 30-60%; + is <300%).

TABLE 7

| | | | 3T3 | Ent. | Ent. A | Ent. | Ent. B |
|---|---|---|---|---|---|---|---|
| | | 3T3 | Max | A | Max | B | Max |
| Compd | | EC₅₀ | Activity | EC₅₀ | Activity | EC₅₀ | Activity |
| No. | Structure | (μM) | (%) | (μM) | (%) | (μM) | (%) |
| 1 | 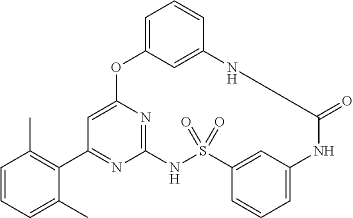 [SMILES: Cc1cccc(C)c1-c1cc2nc(n1)NS(=O)(=O)c1cccc(c1)NC(=O)Nc1cccc(c1)O2] | ++ | +++ | ++ | ++ | | |
| 206 | 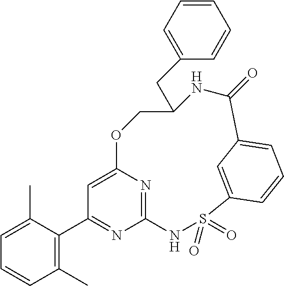 [SMILES: Cc1cccc(C)c1-c1cc2nc(n1)NS(=O)(=O)c1cccc(c1)C(=O)NC(Cc1ccccc1)CO2] | +++ | +++ | +++ | +++ | | |
| 204 | 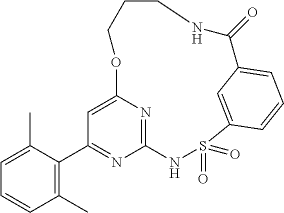 [SMILES: Cc1cccc(C)c1-c1cc2nc(n1)NS(=O)(=O)c1cccc(c1)C(=O)NCCCO2] | ++ | +++ | ND | ++ | | |
| 199 | 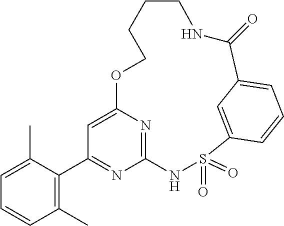 [SMILES: Cc1cccc(C)c1-c1cc2nc(n1)NS(=O)(=O)c1cccc(c1)C(=O)NCCCCO2] | ND | + | ND | ND | | |
| 198 | 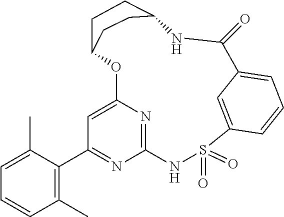 [SMILES: Cc1cccc(C)c1-c1cc2nc(n1)NS(=O)(=O)c1cccc(c1)C(=O)N[C@H]1CC[C@H](CC1)O2] | +++ | +++ | ++ | +++ | | |
| 203 | 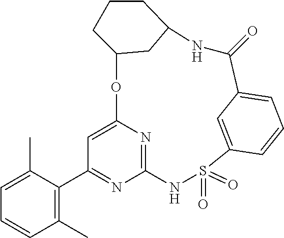 [SMILES: Cc1cccc(C)c1-c1cc2nc(n1)NS(=O)(=O)c1cccc(c1)C(=O)NC1CCCC(C1)O2] | +++ | +++ | ND | ++ | | |
| 202 | 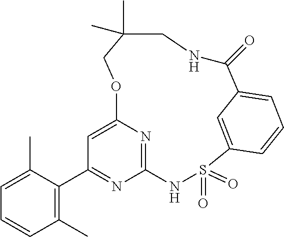 [SMILES: Cc1cccc(C)c1-c1cc2nc(n1)NS(=O)(=O)c1cccc(c1)C(=O)NCC(C)(C)CO2] | +++ | +++ | + | ++ | | |
| 205 | 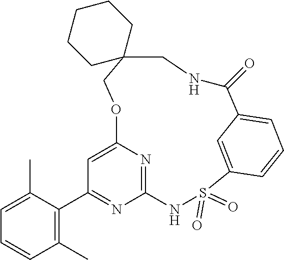 [SMILES: Cc1cccc(C)c1-c1cc2nc(n1)NS(=O)(=O)c1cccc(c1)C(=O)NCC1(CCCCC1)CO2] | | | +++ | +++ | | |
| 79 | 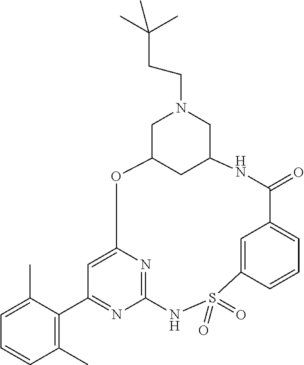 [SMILES: Cc1cccc(C)c1-c1cc2nc(n1)NS(=O)(=O)c1cccc(c1)C(=O)NC1CC(CN(CCC(C)(C)C)C1)O2] | | | +++ | ++ | | |
| 197 | 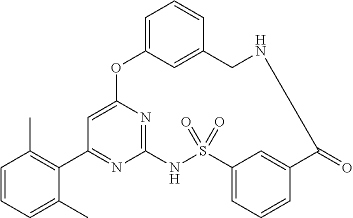 [SMILES: Cc1cccc(C)c1-c1cc2nc(n1)NS(=O)(=O)c1cccc(c1)C(=O)NCc1cccc(c1)O2] | | | + | ++ | | |
| 80 | 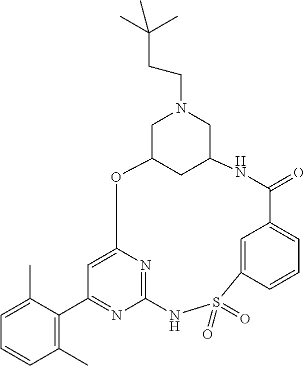 [SMILES: Cc1cccc(C)c1-c1cc2nc(n1)NS(=O)(=O)c1cccc(c1)C(=O)NC1CC(CN(CCC(C)(C)C)C1)O2] | | | ++ | ++ | | |
| 356 | 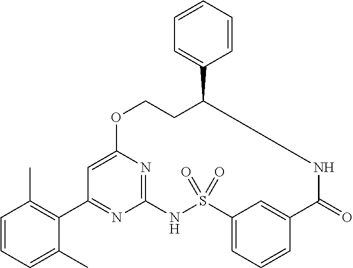 [SMILES: Cc1cccc(C)c1-c1cc2nc(n1)NS(=O)(=O)c1cccc(c1)C(=O)N[C@H](c1ccccc1)CCO2] | | | ND | + | | |
| 201 | 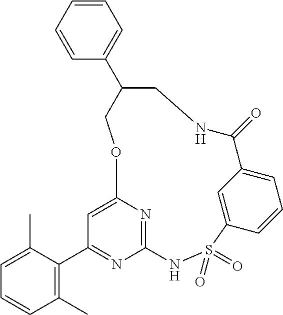 [SMILES: Cc1cccc(C)c1-c1cc2nc(n1)NS(=O)(=O)c1cccc(c1)C(=O)NCC(c1ccccc1)CO2] | | | +++ | + | | |
| 366 | 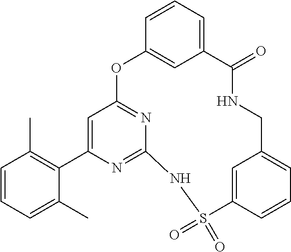 [SMILES: Cc1cccc(C)c1-c1cc2nc(n1)NS(=O)(=O)c1cccc(c1)CNC(=O)c1cccc(c1)O2] | | | ND | + | | |
| 355 | 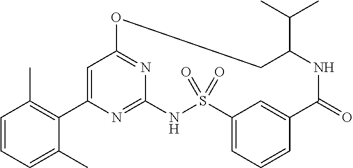 [SMILES: Cc1cccc(C)c1-c1cc2nc(n1)NS(=O)(=O)c1cccc(c1)C(=O)NC(C(C)C)CO2] | | | ++ | ++ | | |
| 200 | 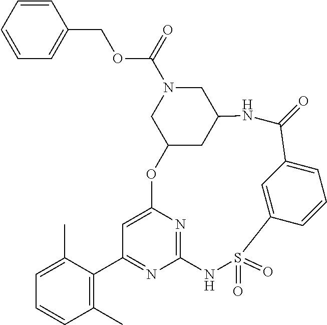 [SMILES: Cc1cccc(C)c1-c1cc2nc(n1)NS(=O)(=O)c1cccc(c1)C(=O)NC1CC(CN(C(=O)OCc3ccccc3)C1)O2] | | | + | ++ | | |
| 196 | 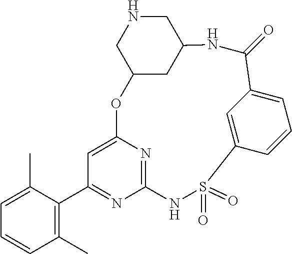 [SMILES: Cc1cccc(C)c1-c1cc2nc(n1)NS(=O)(=O)c1cccc(c1)C(=O)NC1CNCC(C1)O2] | | | ND | + | | |
| 195 | 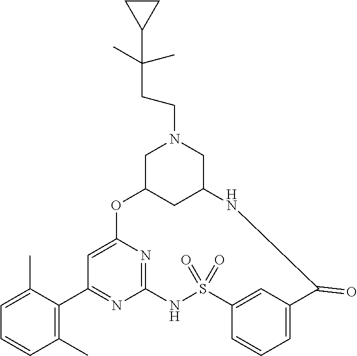 [SMILES: Cc1cccc(C)c1-c1cc2nc(n1)NS(=O)(=O)c1cccc(c1)C(=O)NC1CC(CN(CCC(C)(C)C3CC3)C1)O2] | | | +++ | + | | |
| 194 | 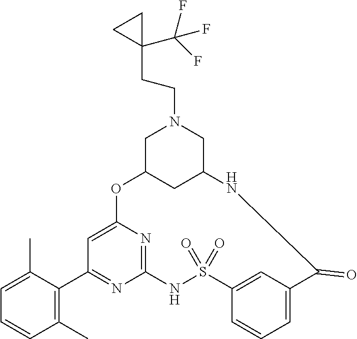 [SMILES: Cc1cccc(C)c1-c1cc2nc(n1)NS(=O)(=O)c1cccc(c1)C(=O)NC1CC(CN(CCC3(C(F)(F)F)CC3)C1)O2] | | | ++ | + | | |
| 193 | 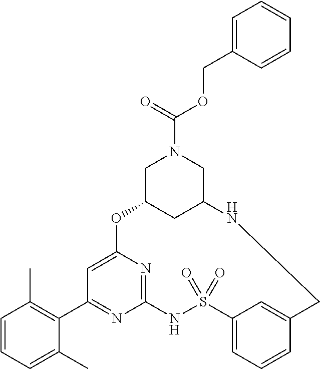 [SMILES: Cc1cccc(C)c1-c1cc2nc(n1)NS(=O)(=O)c1cccc(c1)CNC1C[C@@H](CN(C(=O)OCc3ccccc3)C1)O2] | | | ++ | ++ | | |
| 192 | 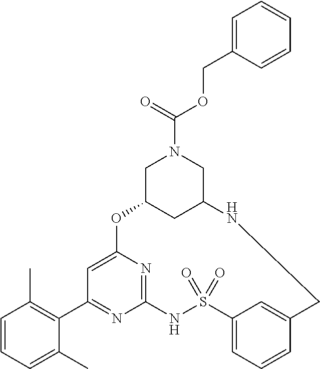 [SMILES: Cc1cccc(C)c1-c1cc2nc(n1)NS(=O)(=O)c1cccc(c1)CNC1C[C@@H](CN(C(=O)OCc3ccccc3)C1)O2] | | | ++ | ++ | | |
| 81 | 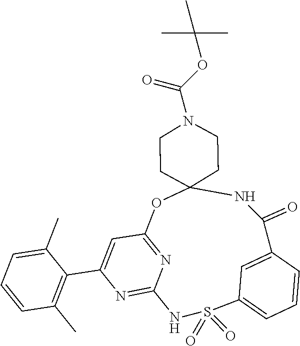 [SMILES: Cc1cccc(C)c1-c1cc2nc(n1)NS(=O)(=O)c1cccc(c1)C(=O)NC1(CCN(C(=O)OC(C)(C)C)CC1)O2] | | | ++ | + | | |
| 491 | 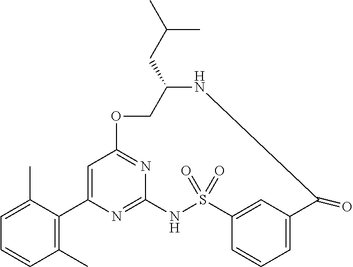 [SMILES: Cc1cccc(C)c1-c1cc2nc(n1)NS(=O)(=O)c1cccc(c1)C(=O)N[C@@H](CC(C)C)CO2] | | | ++ | + | | |
| 82 | 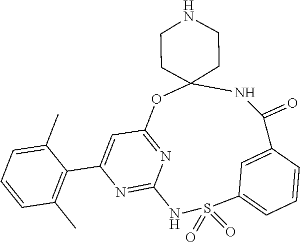 [SMILES: Cc1cccc(C)c1-c1cc2nc(n1)NS(=O)(=O)c1cccc(c1)C(=O)NC1(CCNCC1)O2] | | | ND | + | | |
| 96 | 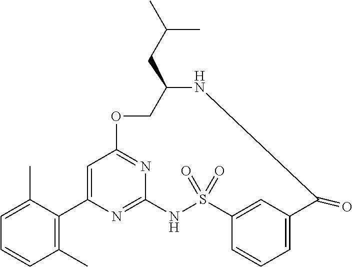 [SMILES: Cc1cccc(C)c1-c1cc2nc(n1)NS(=O)(=O)c1cccc(c1)C(=O)N[C@H](CC(C)C)CO2] | +++ | +++ | +++ | +++ | +++ | +++ |
| 490 | 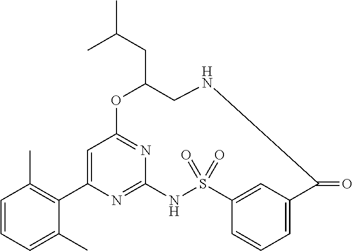 [SMILES: Cc1cccc(C)c1-c1cc2nc(n1)NS(=O)(=O)c1cccc(c1)C(=O)NCC(CC(C)C)O2] | | | ++ | +++ | | |
| 428 | 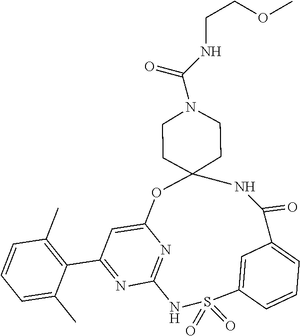 [SMILES: COCCNC(=O)N1CCC2(CC1)NC(=O)c1cccc(c1)S(=O)(=O)Nc1nc(cc(-c3c(C)cccc3C)n1)O2] | | | ND | + | | |
| 427 | 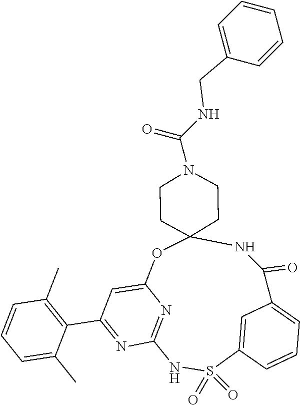 [SMILES: Cc1cccc(C)c1-c1cc2nc(n1)NS(=O)(=O)c1cccc(c1)C(=O)NC1(CCN(C(=O)NCc3ccccc3)CC1)O2] | | | ND | + | | |
| 426 | 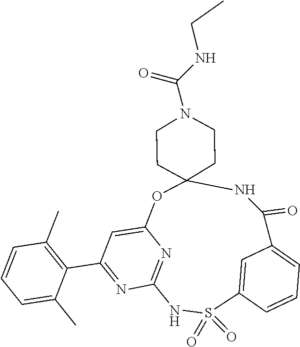 [SMILES: CCNC(=O)N1CCC2(CC1)NC(=O)c1cccc(c1)S(=O)(=O)Nc1nc(cc(-c3c(C)cccc3C)n1)O2] | | | ND | + | | |
| 425 | 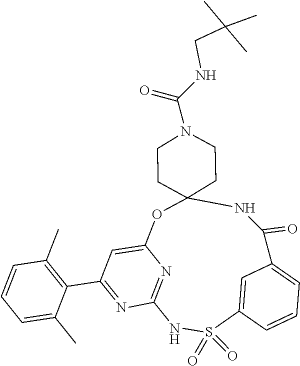 [SMILES: Cc1cccc(C)c1-c1cc2nc(n1)NS(=O)(=O)c1cccc(c1)C(=O)NC1(CCN(C(=O)NCC(C)(C)C)CC1)O2] | | | ND | + | | |
| 446 | 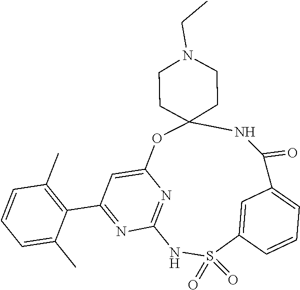 [SMILES: CCN1CCC2(CC1)NC(=O)c1cccc(c1)S(=O)(=O)Nc1nc(cc(-c3c(C)cccc3C)n1)O2] | | | ND | + | | |
| 445 | 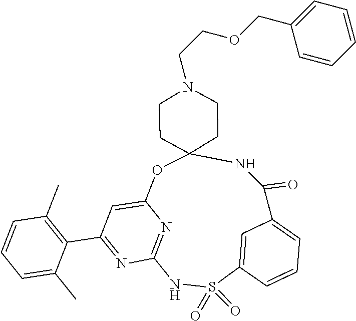 [SMILES: Cc1cccc(C)c1-c1cc2nc(n1)NS(=O)(=O)c1cccc(c1)C(=O)NC1(CCN(CCOCc3ccccc3)CC1)O2] | | | + | ++ | | |
| 444 | 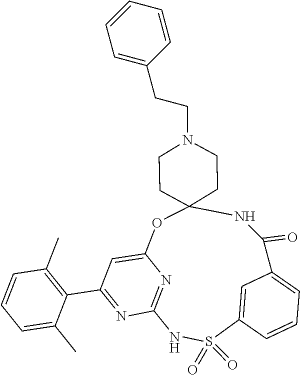 [SMILES: Cc1cccc(C)c1-c1cc2nc(n1)NS(=O)(=O)c1cccc(c1)C(=O)NC1(CCN(CCc3ccccc3)CC1)O2] | | | + | ++ | | |
| 443 | 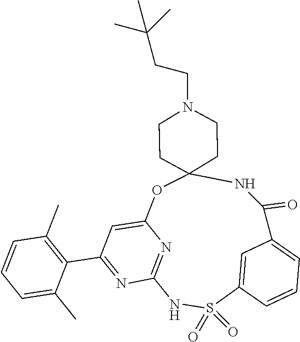 [SMILES: Cc1cccc(C)c1-c1cc2nc(n1)NS(=O)(=O)c1cccc(c1)C(=O)NC1(CCN(CCC(C)(C)C)CC1)O2] | | | ND | ++ | | |
| 482 | 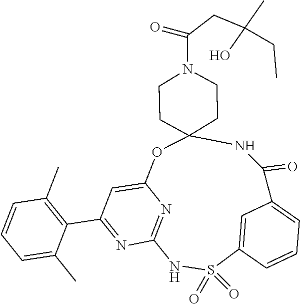 [SMILES: CCC(C)(O)CC(=O)N1CCC2(CC1)NC(=O)c1cccc(c1)S(=O)(=O)Nc1nc(cc(-c3c(C)cccc3C)n1)O2] | | | ND | + | | |
| 481 | 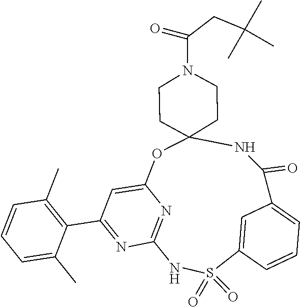 [SMILES: Cc1cccc(C)c1-c1cc2nc(n1)NS(=O)(=O)c1cccc(c1)C(=O)NC1(CCN(C(=O)CC(C)(C)C)CC1)O2] | | | ND | ++ | | |
| 480 | 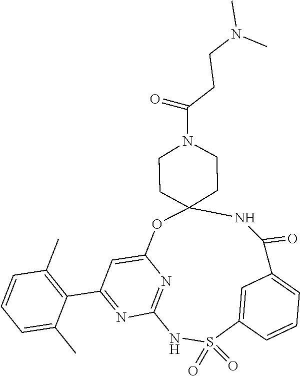 [SMILES: Cc1cccc(C)c1-c1cc2nc(n1)NS(=O)(=O)c1cccc(c1)C(=O)NC1(CCN(C(=O)CCN(C)C)CC1)O2] | | | ND | + | | |
| 479 | 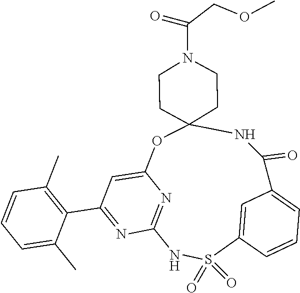 [SMILES: COCC(=O)N1CCC2(CC1)NC(=O)c1cccc(c1)S(=O)(=O)Nc1nc(cc(-c3c(C)cccc3C)n1)O2] | | | ND | + | | |
| 478 | 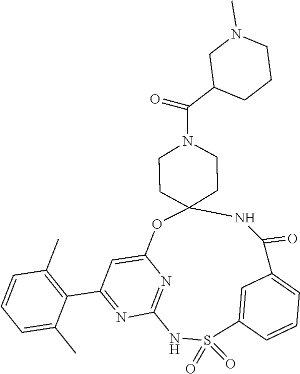 [SMILES: Cc1cccc(C)c1-c1cc2nc(n1)NS(=O)(=O)c1cccc(c1)C(=O)NC1(CCN(C(=O)C3CCCN(C)C3)CC1)O2] | | | ND | + | | |
| 477 | 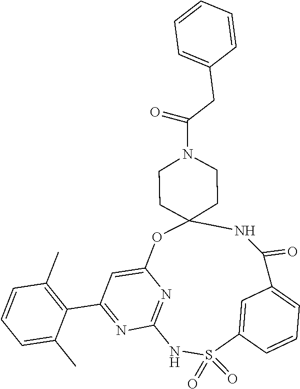 [SMILES: Cc1cccc(C)c1-c1cc2nc(n1)NS(=O)(=O)c1cccc(c1)C(=O)NC1(CCN(C(=O)Cc3ccccc3)CC1)O2] | | | ND | + | | |
| 475 | 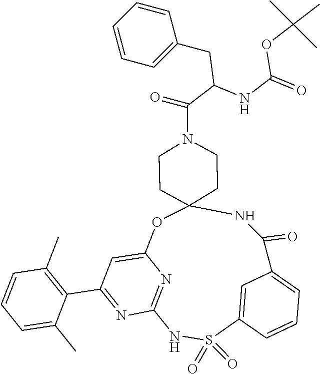 [SMILES: Cc1cccc(C)c1-c1cc2nc(n1)NS(=O)(=O)c1cccc(c1)C(=O)NC1(CCN(C(=O)C(Cc3ccccc3)NC(=O)OC(C)(C)C)CC1)O2] | | | ND | + | | |
| 474 | 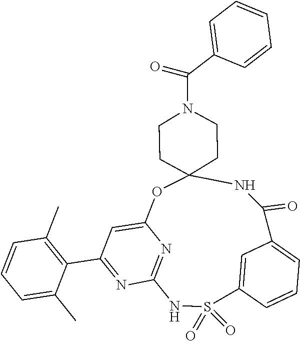 [SMILES: Cc1cccc(C)c1-c1cc2nc(n1)NS(=O)(=O)c1cccc(c1)C(=O)NC1(CCN(C(=O)c3ccccc3)CC1)O2] | | | + | + | | |
| 473 | 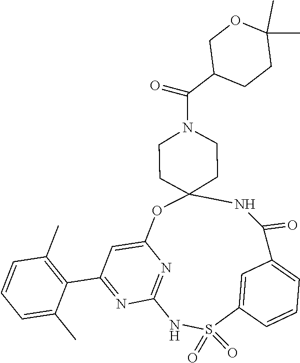 [SMILES: Cc1cccc(C)c1-c1cc2nc(n1)NS(=O)(=O)c1cccc(c1)C(=O)NC1(CCN(C(=O)C3CCC(C)(C)OC3)CC1)O2] | | | ND | + | | |
| 476 | 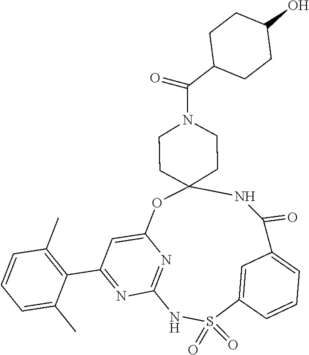 | | | ND | + | | |
| 414 | 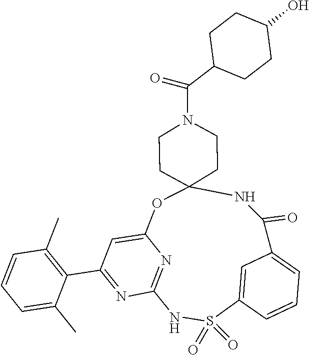 | | | ND | + | | |
| 424 | 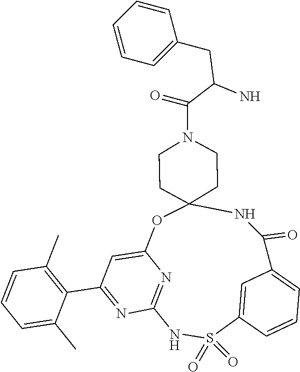 [SMILES: Cc1cccc(C)c1-c1cc2nc(n1)NS(=O)(=O)c1cccc(c1)C(=O)NC1(CCN(C(=O)C(N)Cc3ccccc3)CC1)O2] | | | | | | |
| 429 | 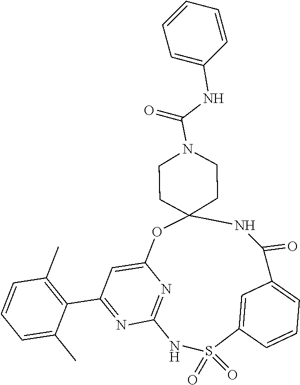 [SMILES: Cc1cccc(C)c1-c1cc2nc(n1)NS(=O)(=O)c1cccc(c1)C(=O)NC1(CCN(C(=O)Nc3ccccc3)CC1)O2] | | | ND | + | | |
| 423 | 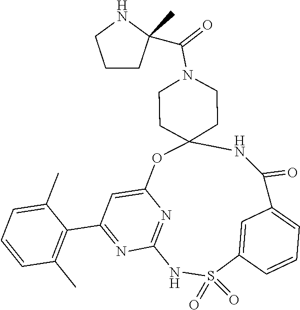 [SMILES: Cc1cccc(C)c1-c1cc2nc(n1)NS(=O)(=O)c1cccc(c1)C(=O)NC1(CCN(C(=O)[C@]3(C)CCCN3)CC1)O2] | | | ND | ND | | |
| 422 | 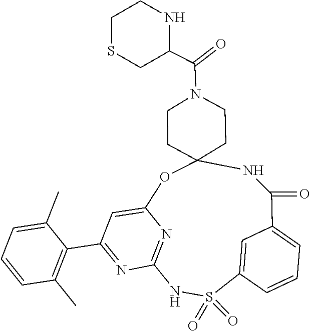 [SMILES: Cc1cccc(C)c1-c1cc2nc(n1)NS(=O)(=O)c1cccc(c1)C(=O)NC1(CCN(C(=O)C3CSCCN3)CC1)O2] | | | ND | + | | |
| 421 | 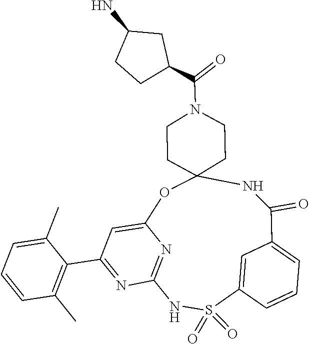 [SMILES: Cc1cccc(C)c1-c1cc2nc(n1)NS(=O)(=O)c1cccc(c1)C(=O)NC1(CCN(C(=O)[C@H]3CC[C@@H](N)C3)CC1)O2] | | | ND | + | | |
| 420 | 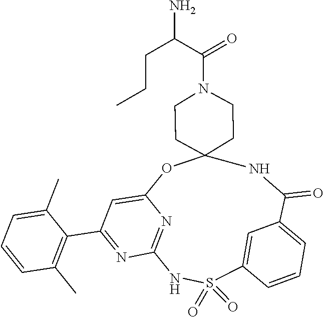 [SMILES: CCCC(N)C(=O)N1CCC2(CC1)NC(=O)c1cccc(c1)S(=O)(=O)Nc1nc(cc(-c3c(C)cccc3C)n1)O2] | | | ND | + | | |
| 419 | 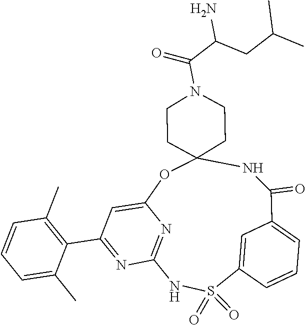 [SMILES: Cc1cccc(C)c1-c1cc2nc(n1)NS(=O)(=O)c1cccc(c1)C(=O)NC1(CCN(C(=O)C(N)CC(C)C)CC1)O2] | | | ND | + | | |
| 418 | 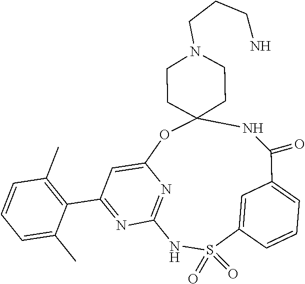 [SMILES: Cc1cccc(C)c1-c1cc2nc(n1)NS(=O)(=O)c1cccc(c1)C(=O)NC1(CCN(CCCN)CC1)O2] | | | ND | + | | |
| 472 | 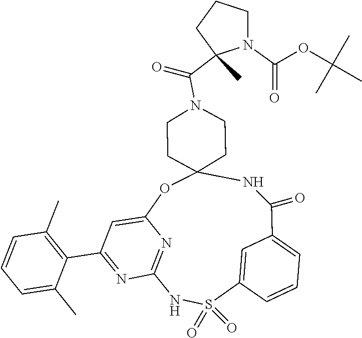 [SMILES: Cc1cccc(C)c1-c1cc2nc(n1)NS(=O)(=O)c1cccc(c1)C(=O)NC1(CCN(C(=O)[C@]3(C)CCCN3C(=O)OC(C)(C)C)CC1)O2] | | | + | ++ | | |
| 471 | 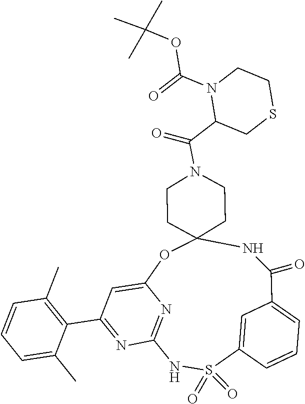 [SMILES: Cc1cccc(C)c1-c1cc2nc(n1)NS(=O)(=O)c1cccc(c1)C(=O)NC1(CCN(C(=O)C3CSCCN3C(=O)OC(C)(C)C)CC1)O2] | | | ND | ++ | | |
| 470 | 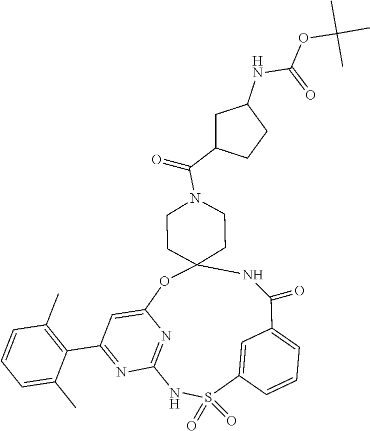 [SMILES: Cc1cccc(C)c1-c1cc2nc(n1)NS(=O)(=O)c1cccc(c1)C(=O)NC1(CCN(C(=O)C3CCC(NC(=O)OC(C)(C)C)C3)CC1)O2] | | | ND | ++ | | |
| 469 | 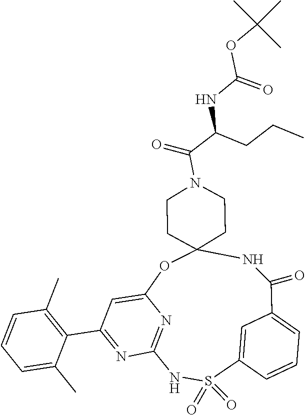 [SMILES: CCC[C@H](NC(=O)OC(C)(C)C)C(=O)N1CCC2(CC1)NC(=O)c1cccc(c1)S(=O)(=O)Nc1nc(cc(-c3c(C)cccc3C)n1)O2] | | | + | ++ | | |
| 468 | 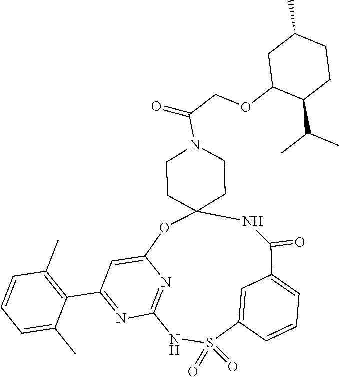 [SMILES: Cc1cccc(C)c1-c1cc2nc(n1)NS(=O)(=O)c1cccc(c1)C(=O)NC1(CCN(C(=O)COC3C[C@H](C)CC[C@H]3C(C)C)CC1)O2] | | | ++ | ++ | | |
| 466 | 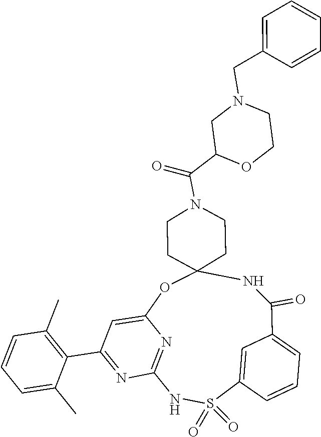 [SMILES: Cc1cccc(C)c1-c1cc2nc(n1)NS(=O)(=O)c1cccc(c1)C(=O)NC1(CCN(C(=O)C3CN(Cc4ccccc4)CCO3)CC1)O2] | | | ND | + | | |
| 465 | 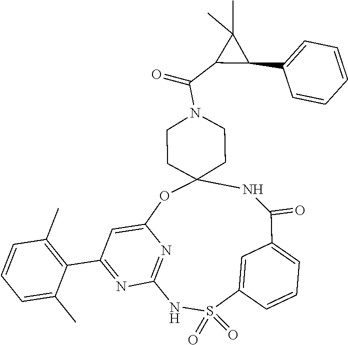 [SMILES: Cc1cccc(C)c1-c1cc2nc(n1)NS(=O)(=O)c1cccc(c1)C(=O)NC1(CCN(C(=O)C3[C@H](c4ccccc4)C3(C)C)CC1)O2] | | | ++ | ++ | | |
| 464 | 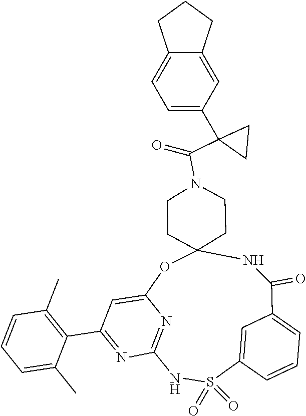 [SMILES: Cc1cccc(C)c1-c1cc2nc(n1)NS(=O)(=O)c1cccc(c1)C(=O)NC1(CCN(C(=O)C3(c4ccc5c(c4)CCC5)CC3)CC1)O2] | | | ++ | +++ | | |
| 463 | 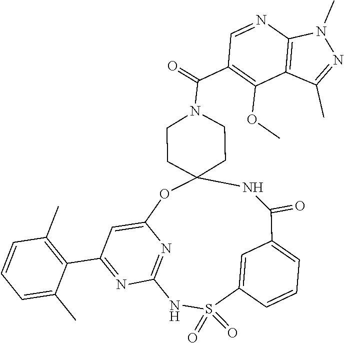 [SMILES: COc1c(C(=O)N2CCC3(CC2)NC(=O)c2cccc(c2)S(=O)(=O)Nc2nc(cc(-c4c(C)cccc4C)n2)O3)cnc2c1c(C)nn2C] | | | ND | + | | |
| 461 | 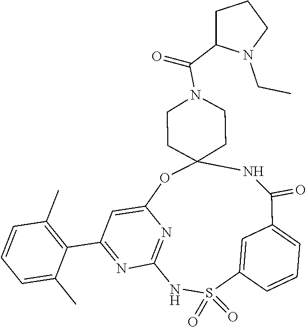 [SMILES: CCN1CCCC1C(=O)N1CCC2(CC1)NC(=O)c1cccc(c1)S(=O)(=O)Nc1nc(cc(-c3c(C)cccc3C)n1)O2] | | | ND | + | | |
| 460 | 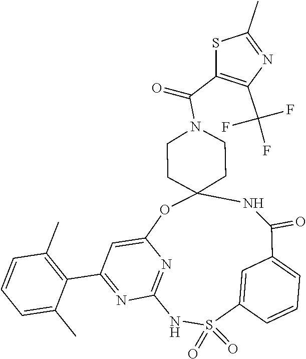 [SMILES: Cc1nc(C(F)(F)F)c(C(=O)N2CCC3(CC2)NC(=O)c2cccc(c2)S(=O)(=O)Nc2nc(cc(-c4c(C)cccc4C)n2)O3)s1] | | | ND | ++ | | |
| 459 | 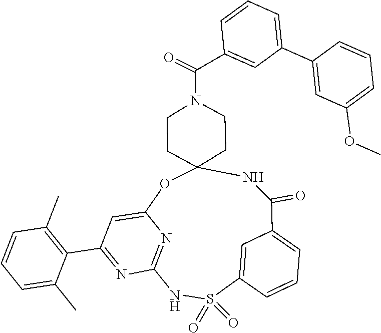 [SMILES: COc1cccc(-c2cccc(C(=O)N3CCC4(CC3)NC(=O)c3cccc(c3)S(=O)(=O)Nc3nc(cc(-c5c(C)cccc5C)n3)O4)c2)c1] | | | ++ | ++ | | |
| 84 | 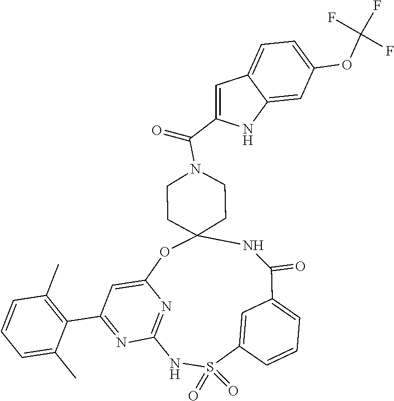 [SMILES: Cc1cccc(C)c1-c1cc2nc(n1)NS(=O)(=O)c1cccc(c1)C(=O)NC1(CCN(C(=O)c3cc4ccc(OC(F)(F)F)cc4[nH]3)CC1)O2] | | | +++ | ++ | | |
| 458 | 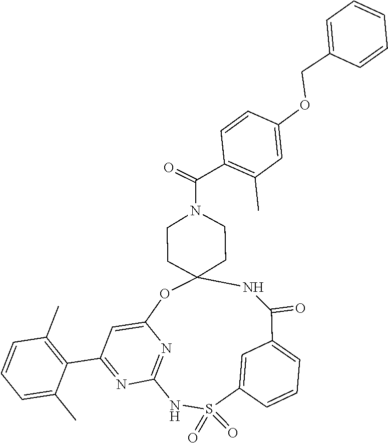 [SMILES: Cc1cc(OCc2ccccc2)ccc1C(=O)N1CCC2(CC1)NC(=O)c1cccc(c1)S(=O)(=O)Nc1nc(cc(-c3c(C)cccc3C)n1)O2] | | | ND | + | | |
| 457 | 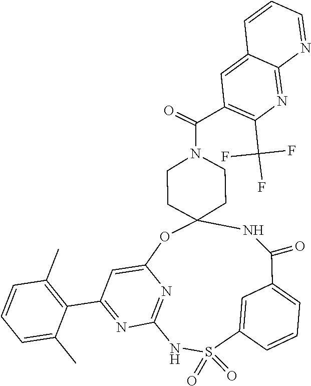 [SMILES: Cc1cccc(C)c1-c1cc2nc(n1)NS(=O)(=O)c1cccc(c1)C(=O)NC1(CCN(C(=O)c3cc4cccnc4nc3C(F)(F)F)CC1)O2] | | | ND | + | | |
| 456 | 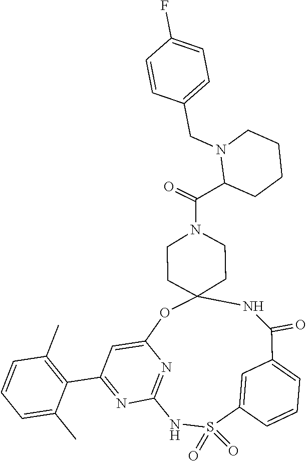 [SMILES: Cc1cccc(C)c1-c1cc2nc(n1)NS(=O)(=O)c1cccc(c1)C(=O)NC1(CCN(C(=O)C3CCCCN3Cc3ccc(F)cc3)CC1)O2] | | | + | ++ | | |
| 455 | 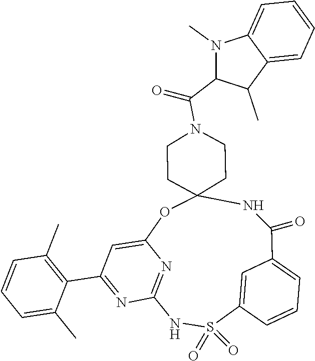 [SMILES: Cc1cccc(C)c1-c1cc2nc(n1)NS(=O)(=O)c1cccc(c1)C(=O)NC1(CCN(C(=O)C3C(C)c4ccccc4N3C)CC1)O2] | | | ++ | ++ | | |
| 454 | 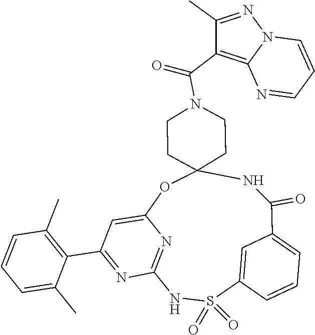 [SMILES: Cc1cccc(C)c1-c1cc2nc(n1)NS(=O)(=O)c1cccc(c1)C(=O)NC1(CCN(C(=O)c3c(C)nn4cccnc34)CC1)O2] | | | ND | + | | |
| 453 | 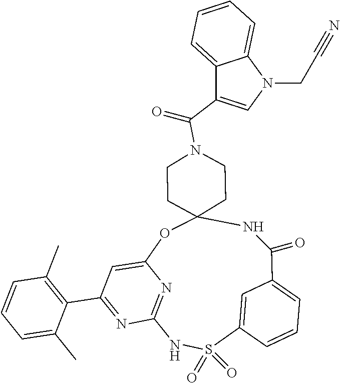 [SMILES: Cc1cccc(C)c1-c1cc2nc(n1)NS(=O)(=O)c1cccc(c1)C(=O)NC1(CCN(C(=O)c3cn(CC#N)c4ccccc34)CC1)O2] | | | ND | + | | |
| 452 | 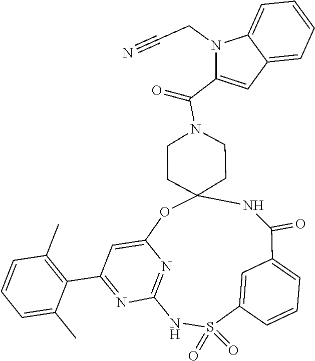 [SMILES: Cc1cccc(C)c1-c1cc2nc(n1)NS(=O)(=O)c1cccc(c1)C(=O)NC1(CCN(C(=O)c3cc4ccccc4n3CC#N)CC1)O2] | | | ++ | ++ | | |
| 451 | 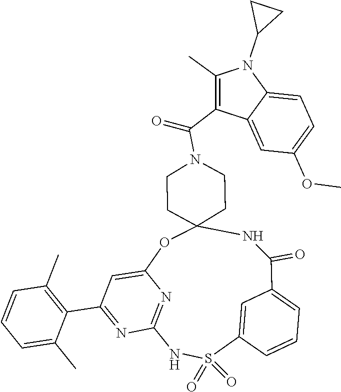 [SMILES: COc1ccc2c(c1)c(C(=O)N1CCC3(CC1)NC(=O)c1cccc(c1)S(=O)(=O)Nc1nc(cc(-c4c(C)cccc4C)n1)O3)c(C)n2C1CC1] | | | + | ++ | | |
| 450 | 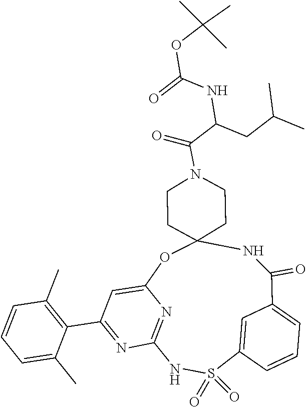 [SMILES: Cc1cccc(C)c1-c1cc2nc(n1)NS(=O)(=O)c1cccc(c1)C(=O)NC1(CCN(C(=O)C(CC(C)C)NC(=O)OC(C)(C)C)CC1)O2] | | | + | ++ | | |
| 85 | 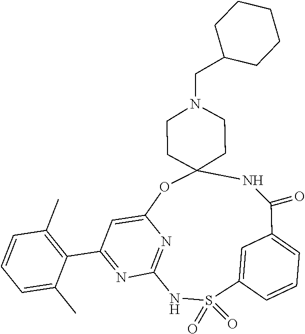 [SMILES: Cc1cccc(C)c1-c1cc2nc(n1)NS(=O)(=O)c1cccc(c1)C(=O)NC1(CCN(CC3CCCCC3)CC1)O2] | | | + | ++ | | |
| 441 | 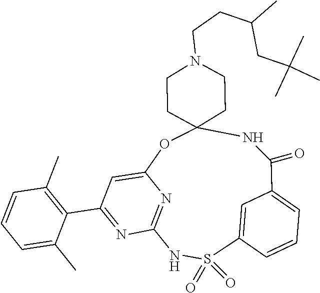 [SMILES: Cc1cccc(C)c1-c1cc2nc(n1)NS(=O)(=O)c1cccc(c1)C(=O)NC1(CCN(CCC(C)CC(C)(C)C)CC1)O2] | | | +++ | +++ | | |
| 83 | 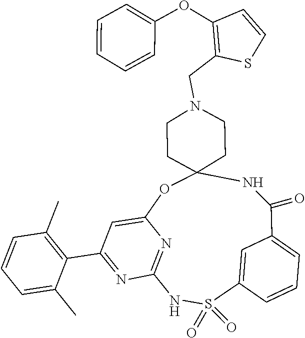 [SMILES: Cc1cccc(C)c1-c1cc2nc(n1)NS(=O)(=O)c1cccc(c1)C(=O)NC1(CCN(Cc3sccc3Oc3ccccc3)CC1)O2] | | | +++ | +++ | | |
| 440 | 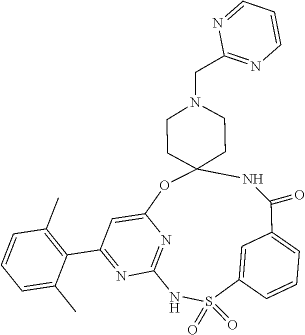 [SMILES: Cc1cccc(C)c1-c1cc2nc(n1)NS(=O)(=O)c1cccc(c1)C(=O)NC1(CCN(Cc3ncccn3)CC1)O2] | | | ND | + | | |
| 437 | 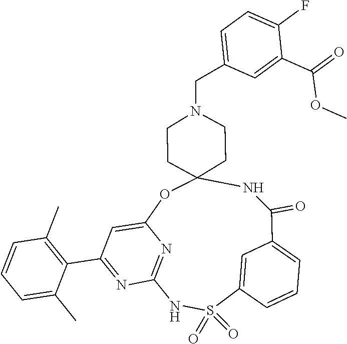 [SMILES: COC(=O)c1cc(CN2CCC3(CC2)NC(=O)c2cccc(c2)S(=O)(=O)Nc2nc(cc(-c4c(C)cccc4C)n2)O3)ccc1F] | | | + | ++ | | |
| 436 | 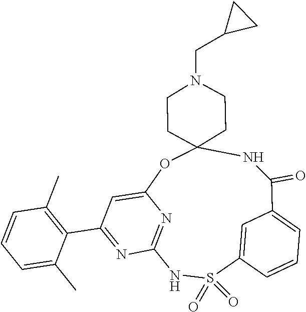 [SMILES: Cc1cccc(C)c1-c1cc2nc(n1)NS(=O)(=O)c1cccc(c1)C(=O)NC1(CCN(CC3CC3)CC1)O2] | | | ND | + | | |
| 434 | 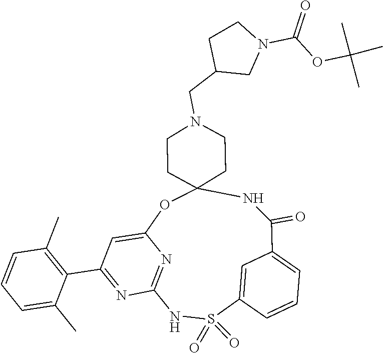 [SMILES: Cc1cccc(C)c1-c1cc2nc(n1)NS(=O)(=O)c1cccc(c1)C(=O)NC1(CCN(CC3CCN(C(=O)OC(C)(C)C)C3)CC1)O2] | | | + | ++ | | |
| 430 | 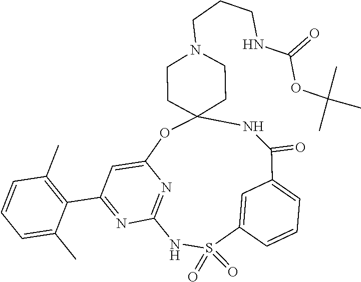 [SMILES: Cc1cccc(C)c1-c1cc2nc(n1)NS(=O)(=O)c1cccc(c1)C(=O)NC1(CCN(CCCNC(=O)OC(C)(C)C)CC1)O2] | | | ND | + | | |
| 439 | 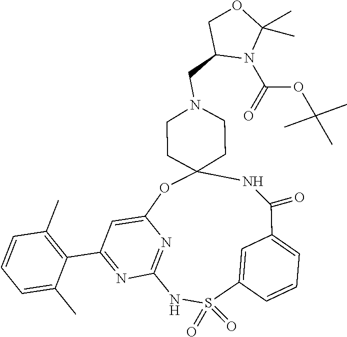 [SMILES: Cc1cccc(C)c1-c1cc2nc(n1)NS(=O)(=O)c1cccc(c1)C(=O)NC1(CCN(C[C@H]3COC(C)(C)N3C(=O)OC(C)(C)C)CC1)O2] | | | + | ++ | | |
| 433 | 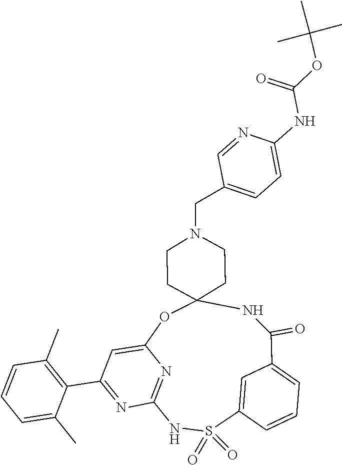 [SMILES: Cc1cccc(C)c1-c1cc2nc(n1)NS(=O)(=O)c1cccc(c1)C(=O)NC1(CCN(Cc3ccc(NC(=O)OC(C)(C)C)nc3)CC1)O2] | | | + | ++ | | |
| 432 | 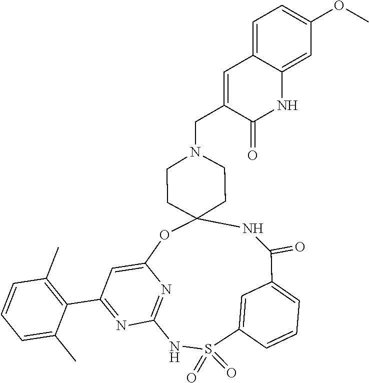 [SMILES: COc1ccc2cc(CN3CCC4(CC3)NC(=O)c3cccc(c3)S(=O)(=O)Nc3nc(cc(-c5c(C)cccc5C)n3)O4)c(=O)[nH]c2c1] | | | ND | + | | |
| 417 | 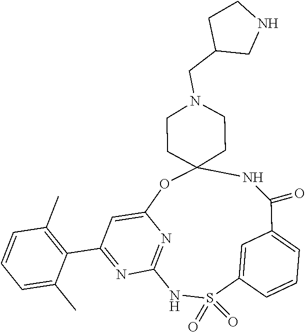 [SMILES: Cc1cccc(C)c1-c1cc2nc(n1)NS(=O)(=O)c1cccc(c1)C(=O)NC1(CCN(CC3CCNC3)CC1)O2] | | | ND | + | | |
| 416 | 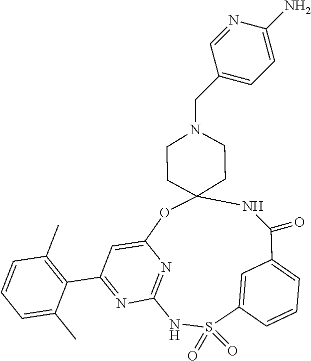 [SMILES: Cc1cccc(C)c1-c1cc2nc(n1)NS(=O)(=O)c1cccc(c1)C(=O)NC1(CCN(Cc3ccc(N)nc3)CC1)O2] | | | ND | + | | |
| 415 | 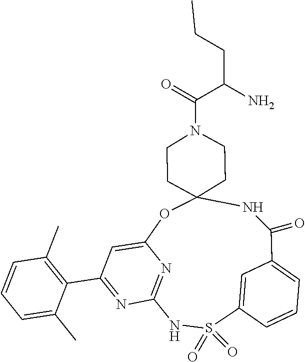 [SMILES: CCCC(N)C(=O)N1CCC2(CC1)NC(=O)c1cccc(c1)S(=O)(=O)Nc1nc(cc(-c3c(C)cccc3C)n1)O2] | | | ND | + | | |
| 442 | 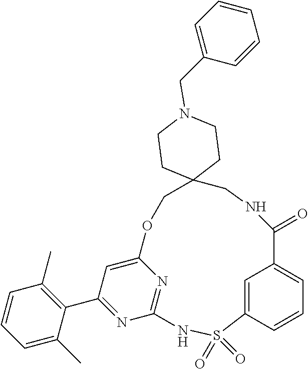 [SMILES: Cc1cccc(C)c1-c1cc2nc(n1)NS(=O)(=O)c1cccc(c1)C(=O)NCC1(CCN(Cc3ccccc3)CC1)CO2] | | | + | + | | |
| 438 | 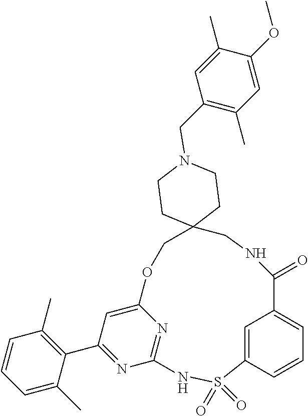 [SMILES: COc1cc(C)c(CN2CCC3(CC2)CNC(=O)c2cccc(c2)S(=O)(=O)Nc2nc(cc(-c4c(C)cccc4C)n2)OC3)cc1C] | | | ++ | ++ | | |
| 431 | 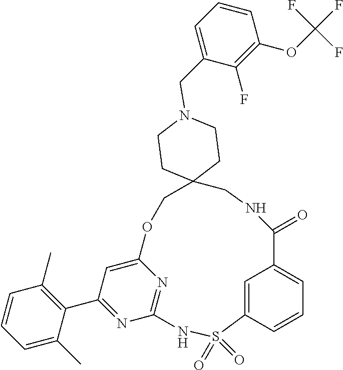 [SMILES: Cc1cccc(C)c1-c1cc2nc(n1)NS(=O)(=O)c1cccc(c1)C(=O)NCC1(CCN(Cc3cccc(OC(F)(F)F)c3F)CC1)CO2] | | | +++ | ++ | | |
| 467 | 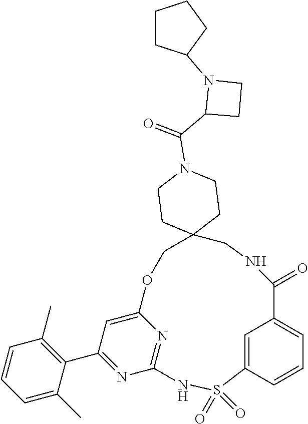 [SMILES: Cc1cccc(C)c1-c1cc2nc(n1)NS(=O)(=O)c1cccc(c1)C(=O)NCC1(CCN(C(=O)C3CCN3C3CCCC3)CC1)CO2] | | | ND | + | | |
| 449 | 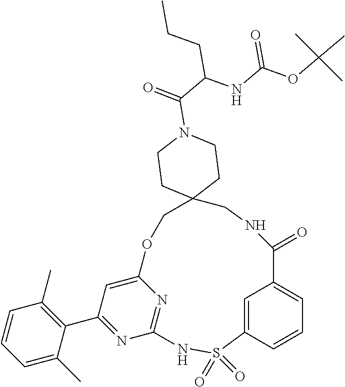 [SMILES: CCCC(NC(=O)OC(C)(C)C)C(=O)N1CCC2(CC1)CNC(=O)c1cccc(c1)S(=O)(=O)Nc1nc(cc(-c3c(C)cccc3C)n1)OC2] | | | ND | ++ | | |
| 435 | 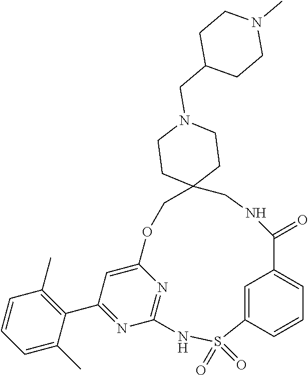 [SMILES: Cc1cccc(C)c1-c1cc2nc(n1)NS(=O)(=O)c1cccc(c1)C(=O)NCC1(CCN(CC3CCN(C)CC3)CC1)CO2] | | | ND | + | | |
| 95 | 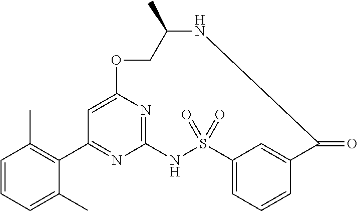 [SMILES: Cc1cccc(C)c1-c1cc2nc(n1)NS(=O)(=O)c1cccc(c1)C(=O)N[C@H](C)CO2] | | | ND | ++ | + | ++ |
| 354 | 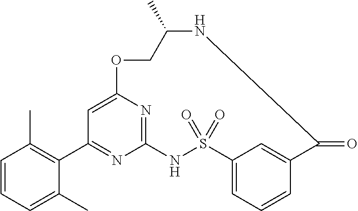 [SMILES: Cc1cccc(C)c1-c1cc2nc(n1)NS(=O)(=O)c1cccc(c1)C(=O)N[C@@H](C)CO2] | | | ND | + | | |
| 353 | 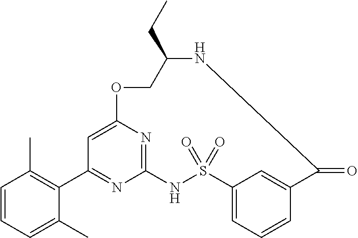 [SMILES: CC[C@@H]1COc2cc(-c3c(C)cccc3C)nc(n2)NS(=O)(=O)c2cccc(c2)C(=O)N1] | | | + | ++ | | |
| 352 | 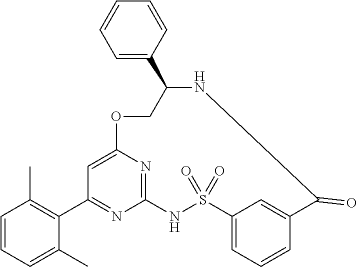 [SMILES: Cc1cccc(C)c1-c1cc2nc(n1)NS(=O)(=O)c1cccc(c1)C(=O)N[C@H](c1ccccc1)CO2] | | | +++ | +++ | | |
| 351 | 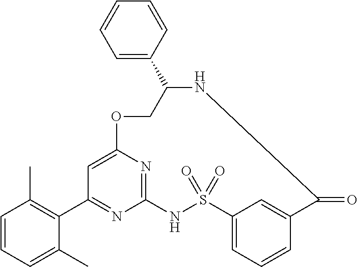 | | | ND | + | | |
| 350 | 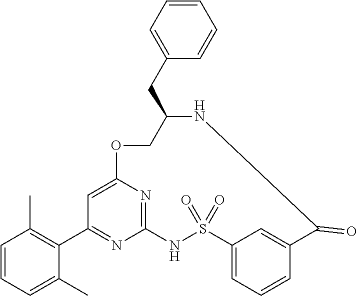 [SMILES: Cc1cccc(C)c1-c1cc2nc(n1)NS(=O)(=O)c1cccc(c1)C(=O)N[C@H](Cc1ccccc1)CO2] | +++ | +++ | +++ | +++ | | |
| 489 | 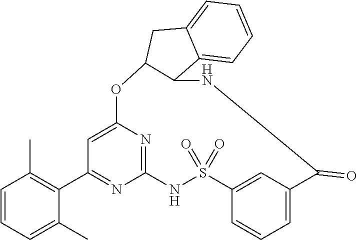 [SMILES: Cc1cccc(C)c1-c1cc2nc(n1)NS(=O)(=O)c1cccc(c1)C(=O)NC1c3ccccc3CC1O2] | | | +++ | +++ | | |
| 462 | 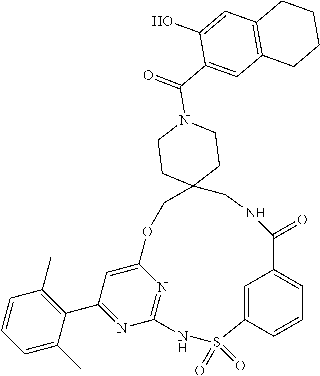 [SMILES: Cc1cccc(C)c1-c1cc2nc(n1)NS(=O)(=O)c1cccc(c1)C(=O)NCC1(CCN(C(=O)c3cc4c(cc3O)CCCC4)CC1)CO2] | | | ND | ++ | | |
| 448 | 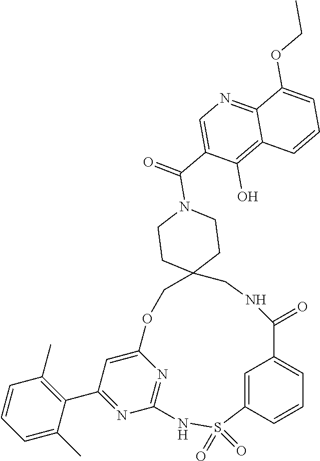 [SMILES: CCOc1cccc2c(O)c(C(=O)N3CCC4(CC3)CNC(=O)c3cccc(c3)S(=O)(=O)Nc3nc(cc(-c5c(C)cccc5C)n3)OC4)cnc12] | | | ND | + | | |
| 447 | 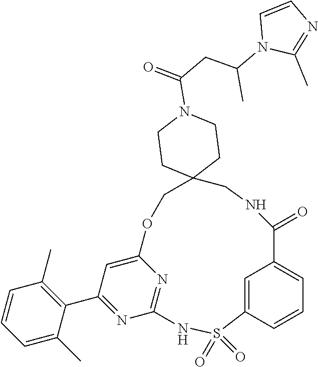 [SMILES: Cc1cccc(C)c1-c1cc2nc(n1)NS(=O)(=O)c1cccc(c1)C(=O)NCC1(CCN(C(=O)CC(C)n3ccnc3C)CC1)CO2] | | | ND | + | | |
| 25 | 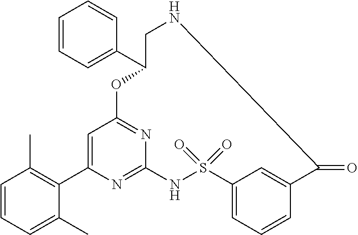 [SMILES: Cc1cccc(C)c1-c1cc2nc(n1)NS(=O)(=O)c1cccc(c1)C(=O)NC[C@@H](c1ccccc1)O2] | | | +++ | +++ | +++ | ++ |
| 26 | 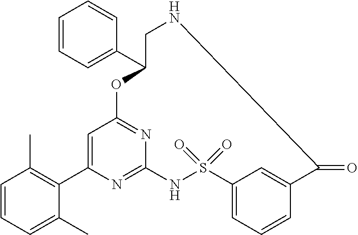 [SMILES: Cc1cccc(C)c1-c1cc2nc(n1)NS(=O)(=O)c1cccc(c1)C(=O)NC[C@H](c1ccccc1)O2] | | | ND | + | | |
| 27 | 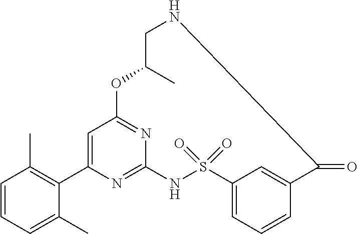 [SMILES: Cc1cccc(C)c1-c1cc2nc(n1)NS(=O)(=O)c1cccc(c1)C(=O)NC[C@H](C)O2] | | | ND | + | | |
| 348 | 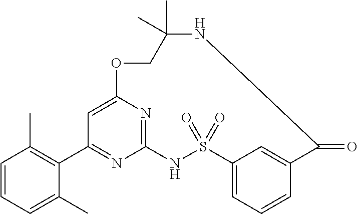 [SMILES: Cc1cccc(C)c1-c1cc2nc(n1)NS(=O)(=O)c1cccc(c1)C(=O)NC(C)(C)CO2] | | | + | +++ | | |
| 112 | 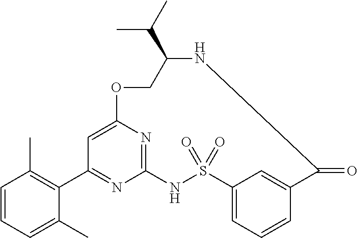 [SMILES: Cc1cccc(C)c1-c1cc2nc(n1)NS(=O)(=O)c1cccc(c1)C(=O)N[C@H](C(C)C)CO2] | | | +++ | +++ | | |
| 347 | 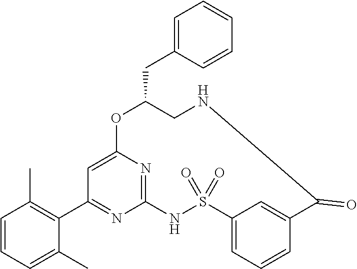 [SMILES: Cc1cccc(C)c1-c1cc2nc(n1)NS(=O)(=O)c1cccc(c1)C(=O)NC[C@@H](Cc1ccccc1)O2] | +++ | +++ | +++ | +++ | | |
| 346 | 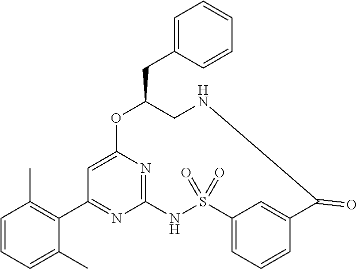 [SMILES: Cc1cccc(C)c1-c1cc2nc(n1)NS(=O)(=O)c1cccc(c1)C(=O)NC[C@H](Cc1ccccc1)O2] | | | +++ | ++ | | |
| 488 | 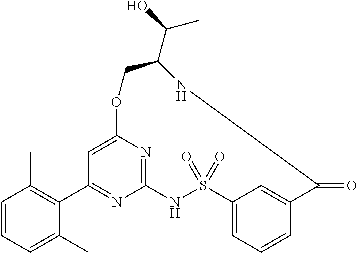 [SMILES: Cc1cccc(C)c1-c1cc2nc(n1)NS(=O)(=O)c1cccc(c1)C(=O)N[C@H]([C@H](C)O)CO2] | | | ND | + | | |
| 349 | 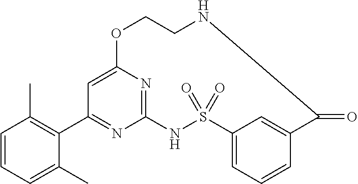 [SMILES: Cc1cccc(C)c1-c1cc2nc(n1)NS(=O)(=O)c1cccc(c1)C(=O)NCCO2] | | | ND | + | | |
| 28 | 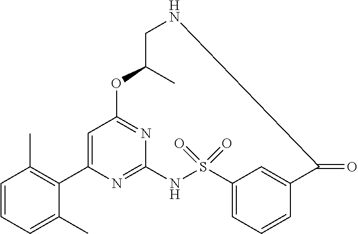 [SMILES: Cc1cccc(C)c1-c1cc2nc(n1)NS(=O)(=O)c1cccc(c1)C(=O)NC[C@@H](C)O2] | | | + | +++ | + | ++ |
| 29 | 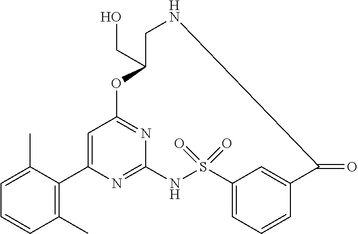 [SMILES: Cc1cccc(C)c1-c1cc2nc(n1)NS(=O)(=O)c1cccc(c1)C(=O)NC[C@H](CO)O2] | | | ND | + | | |
| 487 | 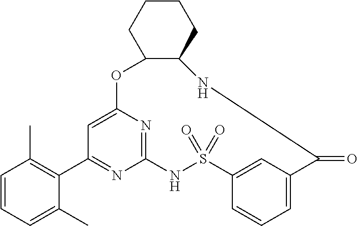 | | | +++ | +++ | | |
| 486 | 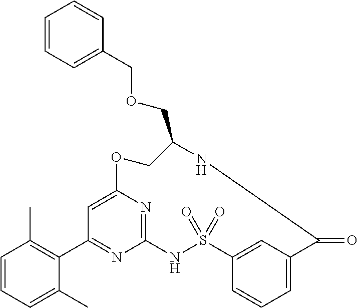 [SMILES: Cc1cccc(C)c1-c1cc2nc(n1)NS(=O)(=O)c1cccc(c1)C(=O)N[C@H](COCc1ccccc1)CO2] | | | ++ | +++ | | |
| 340 | 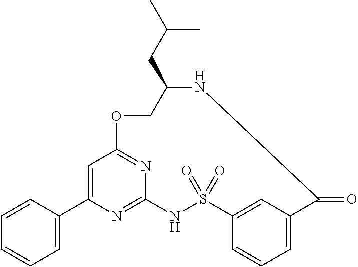 [SMILES: CC(C)C[C@@H]1COc2cc(-c3ccccc3)nc(n2)NS(=O)(=O)c2cccc(c2)C(=O)N1] | | | + | ++ | | |
| 333 | 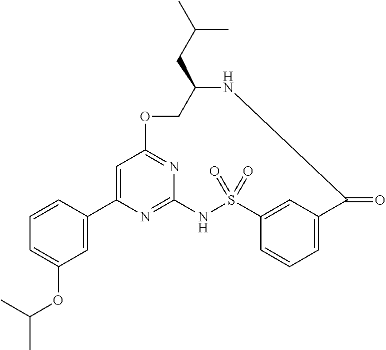 [SMILES: CC(C)C[C@@H]1COc2cc(-c3cccc(OC(C)C)c3)nc(n2)NS(=O)(=O)c2cccc(c2)C(=O)N1] | | | ND | + | | |
| 104 | 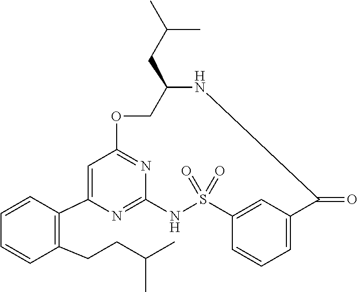 [SMILES: CC(C)CCc1ccccc1-c1cc2nc(n1)NS(=O)(=O)c1cccc(c1)C(=O)N[C@H](CC(C)C)CO2] | | | +++ | ++ | | |
| 332 | 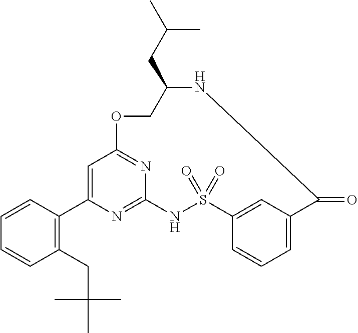 [SMILES: CC(C)C[C@@H]1COc2cc(-c3ccccc3CC(C)(C)C)nc(n2)NS(=O)(=O)c2cccc(c2)C(=O)N1] | | | +++ | ++ | | |
| 331 | 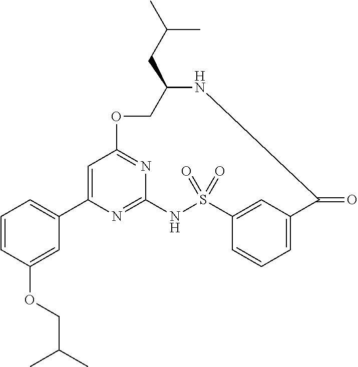 [SMILES: CC(C)COc1cccc(-c2cc3nc(n2)NS(=O)(=O)c2cccc(c2)C(=O)N[C@H](CC(C)C)CO3)c1] | | | ++ | + | | |
| 330 | 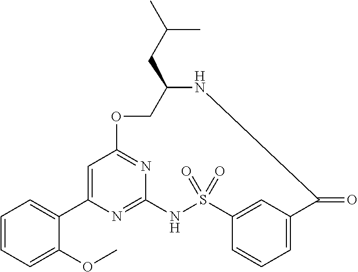 [SMILES: COc1ccccc1-c1cc2nc(n1)NS(=O)(=O)c1cccc(c1)C(=O)N[C@H](CC(C)C)CO2] | | | ND | ++ | | |
| 103 | 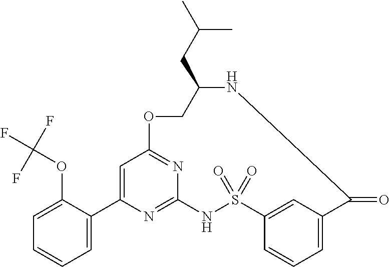 [SMILES: CC(C)C[C@@H]1COc2cc(-c3ccccc3OC(F)(F)F)nc(n2)NS(=O)(=O)c2cccc(c2)C(=O)N1] | | | +++ | +++ | | |
| 105 | 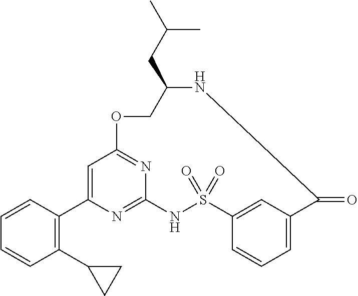 [SMILES: CC(C)C[C@@H]1COc2cc(-c3ccccc3C3CC3)nc(n2)NS(=O)(=O)c2cccc(c2)C(=O)N1] | | | +++ | +++ | | |
| 345 | 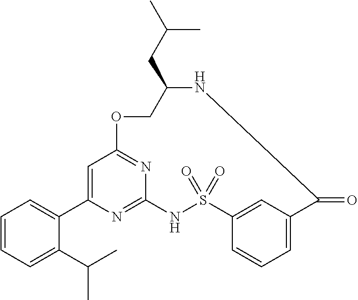 [SMILES: CC(C)C[C@@H]1COc2cc(-c3ccccc3C(C)C)nc(n2)NS(=O)(=O)c2cccc(c2)C(=O)N1] | | | +++ | +++ | | |
| 344 | 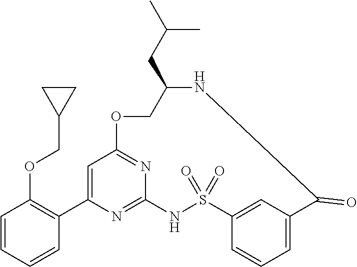 [SMILES: CC(C)C[C@@H]1COc2cc(-c3ccccc3OCC3CC3)nc(n2)NS(=O)(=O)c2cccc(c2)C(=O)N1] | | | ++ | ++ | | |
| 343 | 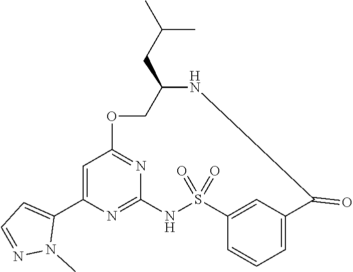 [SMILES: CC(C)C[C@@H]1COc2cc(-c3ccnn3C)nc(n2)NS(=O)(=O)c2cccc(c2)C(=O)N1] | | | ND | + | | |
| 342 | 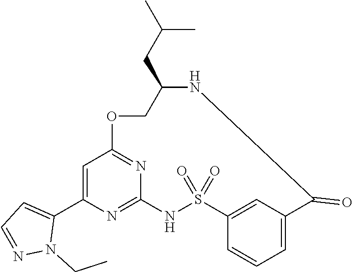 [SMILES: CCn1nccc1-c1cc2nc(n1)NS(=O)(=O)c1cccc(c1)C(=O)N[C@H](CC(C)C)CO2] | | | ND | + | | |
| 341 | 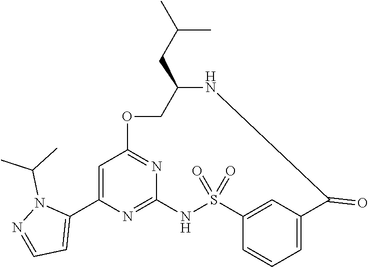 [SMILES: CC(C)C[C@@H]1COc2cc(-c3ccnn3C(C)C)nc(n2)NS(=O)(=O)c2cccc(c2)C(=O)N1] | | | ND | ++ | | |
| 102 | 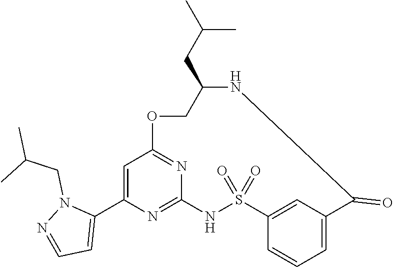 [SMILES: CC(C)C[C@@H]1COc2cc(-c3ccnn3CC(C)C)nc(n2)NS(=O)(=O)c2cccc(c2)C(=O)N1] | | | + | ++ | | |
| 339 | 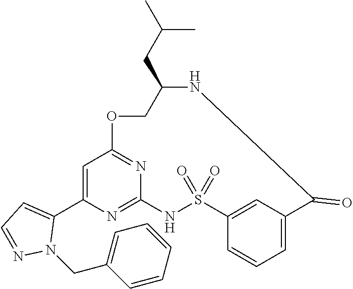 [SMILES: CC(C)C[C@@H]1COc2cc(-c3ccnn3Cc3ccccc3)nc(n2)NS(=O)(=O)c2cccc(c2)C(=O)N1] | | | ND | + | | |
| 338 | 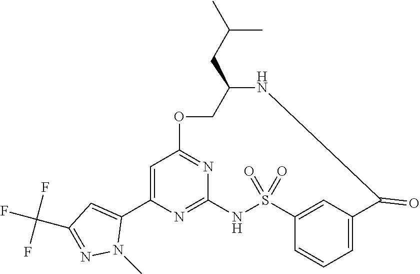 [SMILES: CC(C)C[C@@H]1COc2cc(-c3cc(C(F)(F)F)nn3C)nc(n2)NS(=O)(=O)c2cccc(c2)C(=O)N1] | | | + | + | | |
| 337 | 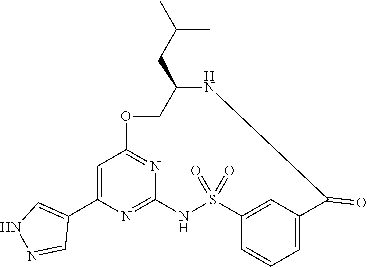 [SMILES: CC(C)C[C@@H]1COc2cc(-c3cn[nH]c3)nc(n2)NS(=O)(=O)c2cccc(c2)C(=O)N1] | | | ND | + | | |
| 336 | 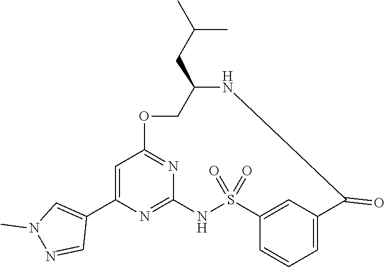 [SMILES: CC(C)C[C@@H]1COc2cc(-c3cnn(C)c3)nc(n2)NS(=O)(=O)c2cccc(c2)C(=O)N1] | | | ND | + | | |
| 335 | 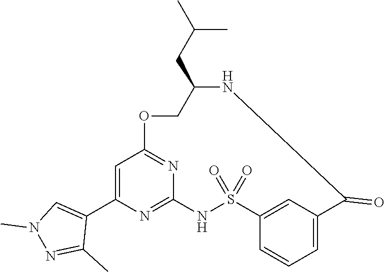 [SMILES: Cc1nn(C)cc1-c1cc2nc(n1)NS(=O)(=O)c1cccc(c1)C(=O)N[C@H](CC(C)C)CO2] | | | ND | + | | |
| 334 | 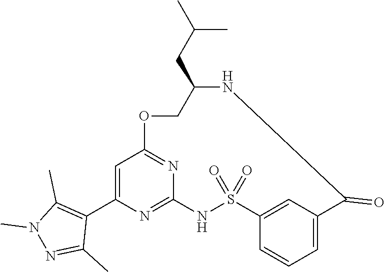 [SMILES: Cc1nn(C)c(C)c1-c1cc2nc(n1)NS(=O)(=O)c1cccc(c1)C(=O)N[C@H](CC(C)C)CO2] | | | ND | + | | |
| 485 | 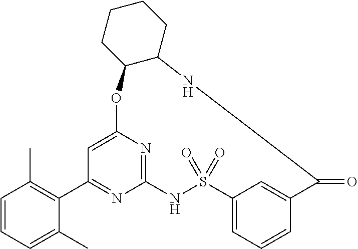 [SMILES: Cc1cccc(C)c1-c1cc2nc(n1)NS(=O)(=O)c1cccc(c1)C(=O)NC1CCCC[C@@H]1O2] | | | ++ | + | | |
| 97 | 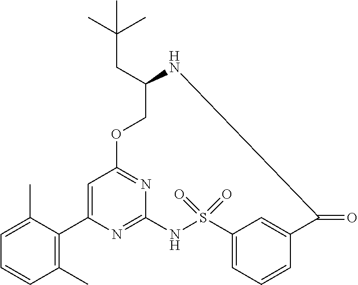 [SMILES: Cc1cccc(C)c1-c1cc2nc(n1)NS(=O)(=O)c1cccc(c1)C(=O)N[C@H](CC(C)(C)C)CO2] | +++ | +++ | +++ | +++ | +++ | +++ |
| 113 | 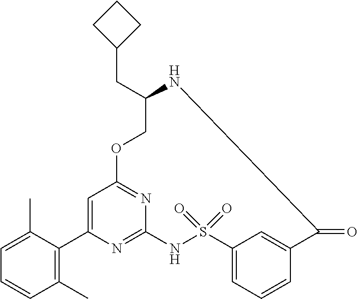 [SMILES: Cc1cccc(C)c1-c1cc2nc(n1)NS(=O)(=O)c1cccc(c1)C(=O)N[C@H](CC1CCC1)CO2] | +++ | +++ | +++ | +++ | | |
| 191 | 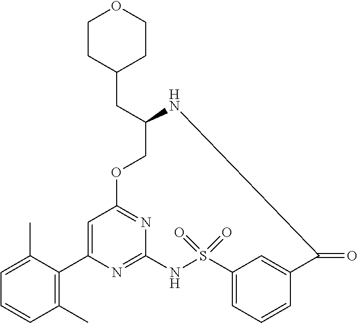 [SMILES: Cc1cccc(C)c1-c1cc2nc(n1)NS(=O)(=O)c1cccc(c1)C(=O)N[C@H](CC1CCOCC1)CO2] | | | + | +++ | | |
| 329 | 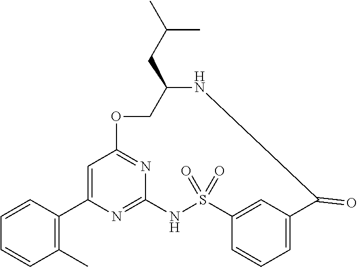 [SMILES: Cc1ccccc1-c1cc2nc(n1)NS(=O)(=O)c1cccc(c1)C(=O)N[C@H](CC(C)C)CO2] | +++ | +++ | +++ | +++ | | |
| 328 | 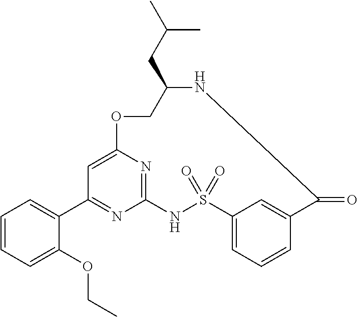 [SMILES: CCOc1ccccc1-c1cc2nc(n1)NS(=O)(=O)c1cccc(c1)C(=O)N[C@H](CC(C)C)CO2] | | | ++ | ++ | | |
| 318 | 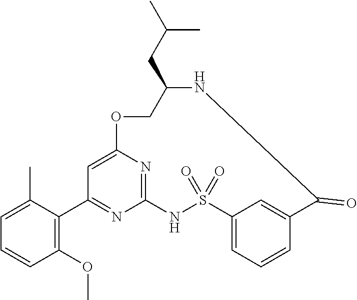 [SMILES: COc1cccc(C)c1-c1cc2nc(n1)NS(=O)(=O)c1cccc(c1)C(=O)N[C@H](CC(C)C)CO2] | +++ | +++ | ++ | +++ | | |
| 317 | 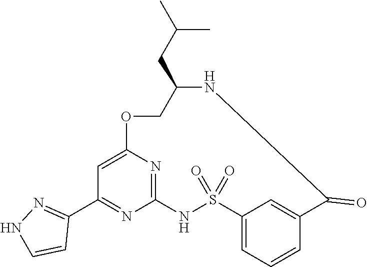 [SMILES: CC(C)C[C@@H]1COc2cc(-c3cc[nH]n3)nc(n2)NS(=O)(=O)c2cccc(c2)C(=O)N1] | | | ND | + | | |
| 327 | 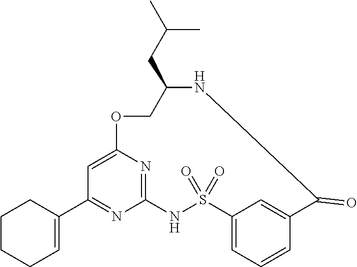 [SMILES: CC(C)C[C@@H]1COc2cc(C3=CCCCC3)nc(n2)NS(=O)(=O)c2cccc(c2)C(=O)N1] | | | ++ | ++ | | |
| 326 | 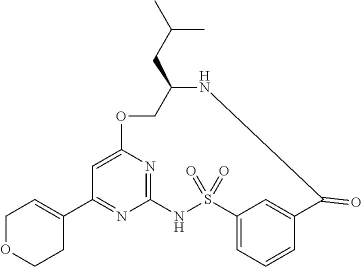 [SMILES: CC(C)C[C@@H]1COc2cc(C3=CCOCC3)nc(n2)NS(=O)(=O)c2cccc(c2)C(=O)N1] | | | ND | + | | |
| 325 | 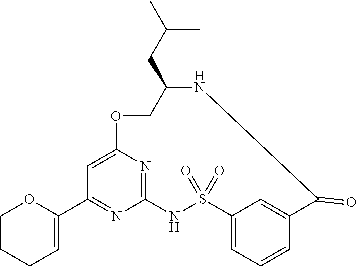 [SMILES: CC(C)C[C@@H]1COc2cc(C3=CCCCO3)nc(n2)NS(=O)(=O)c2cccc(c2)C(=O)N1] | | | ND | + | | |
| 324 | 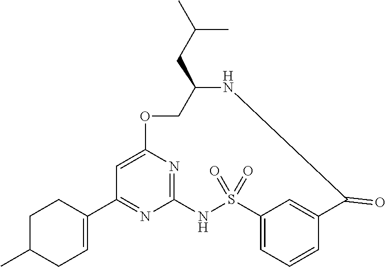 [SMILES: CC(C)C[C@@H]1COc2cc(C3=CCC(C)CC3)nc(n2)NS(=O)(=O)c2cccc(c2)C(=O)N1] | | | ++ | ++ | | |
| 323 | 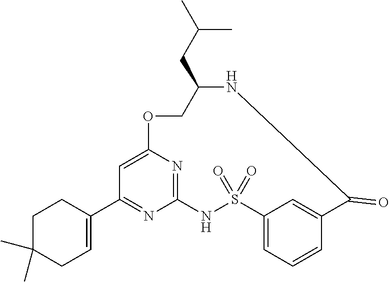 [SMILES: CC(C)C[C@@H]1COc2cc(C3=CCC(C)(C)CC3)nc(n2)NS(=O)(=O)c2cccc(c2)C(=O)N1] | | | ++ | ++ | | |
| 322 | 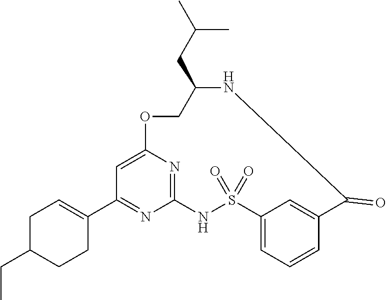 [SMILES: CCC1CC=C(c2cc3nc(n2)NS(=O)(=O)c2cccc(c2)C(=O)N[C@H](CC(C)C)CO3)CC1] | | | ++ | ++ | | |
| 321 | 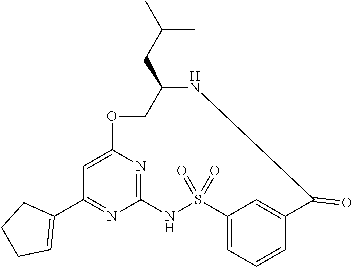 [SMILES: CC(C)C[C@@H]1COc2cc(C3=CCCC3)nc(n2)NS(=O)(=O)c2cccc(c2)C(=O)N1] | | | + | + | | |
| 320 | 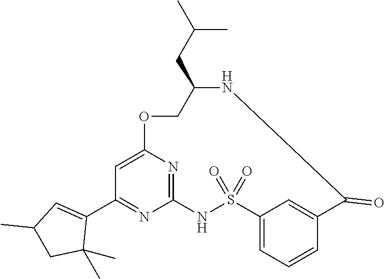 [SMILES: CC(C)C[C@@H]1COc2cc(C3=CC(C)CC3(C)C)nc(n2)NS(=O)(=O)c2cccc(c2)C(=O)N1] | | | +++ | ++ | | |
| 319 | 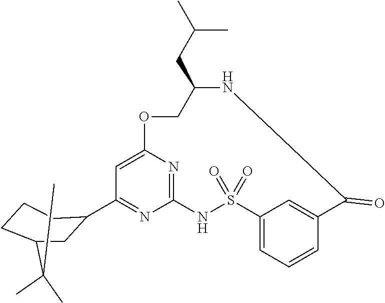 [SMILES: CC(C)C[C@@H]1COc2cc(C3CCCC(C(C)(C)C)C3)nc(n2)NS(=O)(=O)c2cccc(c2)C(=O)N1] | | | +++ | ++ | | |
| 114 | 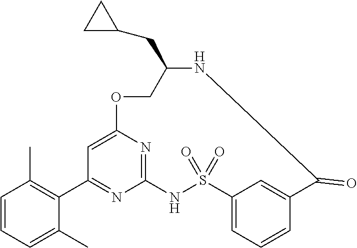 [SMILES: Cc1cccc(C)c1-c1cc2nc(n1)NS(=O)(=O)c1cccc(c1)C(=O)N[C@H](CC1CC1)CO2] | | | +++ | +++ | | |
| 365 | 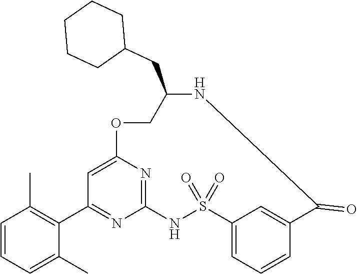 [SMILES: Cc1cccc(C)c1-c1cc2nc(n1)NS(=O)(=O)c1cccc(c1)C(=O)N[C@H](CC1CCCCC1)CO2] | +++ | +++ | +++ | +++ | +++ | +++ |
| 46 | 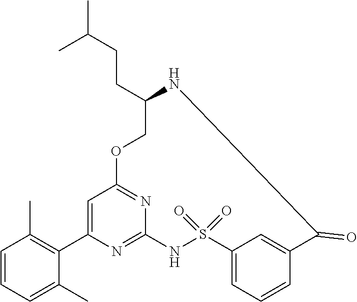 [SMILES: Cc1cccc(C)c1-c1cc2nc(n1)NS(=O)(=O)c1cccc(c1)C(=O)N[C@H](CCC(C)C)CO2] | +++ | +++ | +++ | +++ | | |
| 162 | 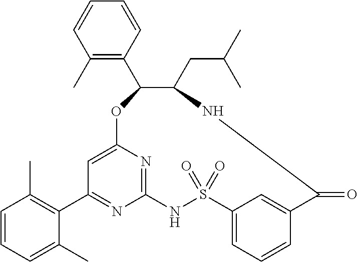 [SMILES: Cc1ccccc1[C@@H]1Oc2cc(-c3c(C)cccc3C)nc(n2)NS(=O)(=O)c2cccc(c2)C(=O)N[C@@H]1CC(C)C] | | | +++ | + | | |
| 161 | 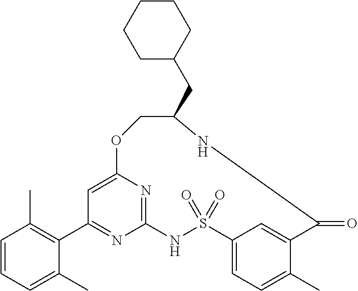 [SMILES: Cc1ccc2cc1C(=O)N[C@H](CC1CCCCC1)COc1cc(-c3c(C)cccc3C)nc(n1)NS2(=O)=O] | + | +++ | ND | + | | |
| 160 | 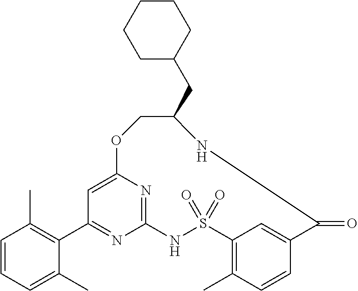 [SMILES: Cc1ccc2cc1S(=O)(=O)Nc1nc(cc(-c3c(C)cccc3C)n1)OC[C@@H](CC1CCCCC1)NC2=O] | +++ | +++ | ++ | ++ | | |
| 364 | 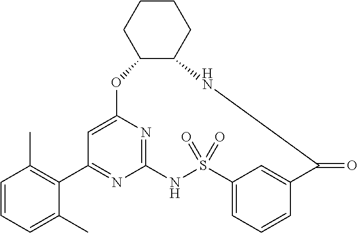 [SMILES: Cc1cccc(C)c1-c1cc2nc(n1)NS(=O)(=O)c1cccc(c1)C(=O)N[C@H]1CCCC[C@H]1O2] | | | + | ++ | | |
| 363 | 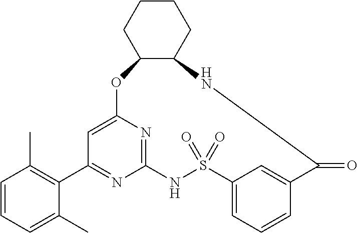 [SMILES: Cc1cccc(C)c1-c1cc2nc(n1)NS(=O)(=O)c1cccc(c1)C(=O)N[C@@H]1CCCC[C@@H]1O2] | | | ++ | ++ | | |
| 316 | 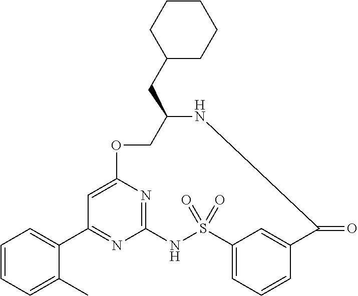 [SMILES: Cc1ccccc1-c1cc2nc(n1)NS(=O)(=O)c1cccc(c1)C(=O)N[C@H](CC1CCCCC1)CO2] | +++ | +++ | +++ | +++ | | |
| 492 | 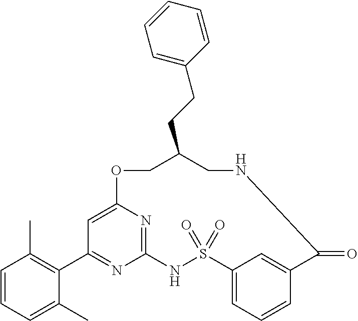 [SMILES: Cc1cccc(C)c1-c1cc2nc(n1)NS(=O)(=O)c1cccc(c1)C(=O)NC[C@H](CCc1ccccc1)CO2] | | | ++ | +++ | | |
| 74 | 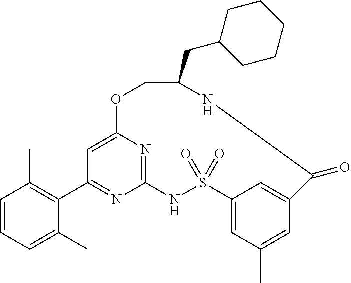 [SMILES: Cc1cc2cc(c1)S(=O)(=O)Nc1nc(cc(-c3c(C)cccc3C)n1)OC[C@@H](CC1CCCCC1)NC2=O] | +++ | +++ | +++ | +++ | | |

The following table represents CFTR modulating activity for representative compounds of the disclosure generated using one or more of the assays disclosed herein (EC₅₀: +++ is <1 μM; ++ is 1−<3 μM; + is 3−<30 μM; and ND is “not detected in this assay.” % Activity: +++ is >60%; ++ is 306%; + is <30%).

TABLE 8
Biological activity data

| | | | 3T3 | Ent. | Ent. A | Ent. | Ent. B |
|---|---|---|---|---|---|---|---|
| | | 3T3 | Max | A | Max | B | Max |
| Compound | | EC₅₀ | Activity | EC50 | Activity | EC50 | Activity |
| number | Structure | (μM) | (%) | (uM) | (%) | (uM) | (%) |
| 412 | 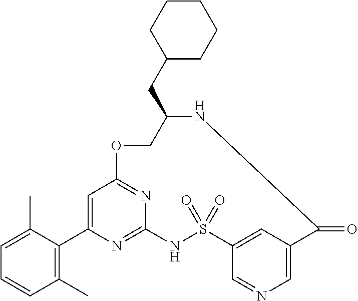 [SMILES: Cc1cccc(C)c1-c1cc2nc(n1)NS(=O)(=O)c1cncc(c1)C(=O)N[C@H](CC1CCCCC1)CO2] | +++ | +++ | +++ | +++ | | |
| 362 | 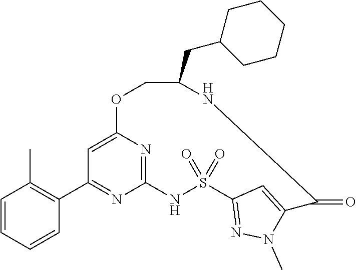 [SMILES: Cc1ccccc1-c1cc2nc(n1)NS(=O)(=O)c1cc(n(C)n1)C(=O)N[C@H](CC1CCCCC1)CO2] | | | ND | ND | | |
| 413 | 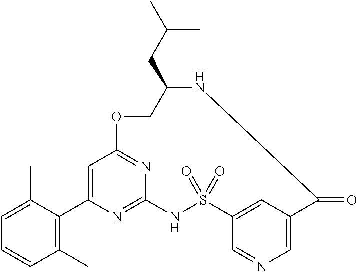 [SMILES: Cc1cccc(C)c1-c1cc2nc(n1)NS(=O)(=O)c1cncc(c1)C(=O)N[C@H](CC(C)C)CO2] | | | ++ | +++ | | |
| 144 | 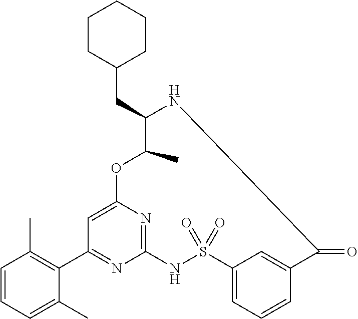 [SMILES: Cc1cccc(C)c1-c1cc2nc(n1)NS(=O)(=O)c1cccc(c1)C(=O)N[C@H](CC1CCCCC1)[C@@H](C)O2] | | | +++ | +++ | | |
| 143 | 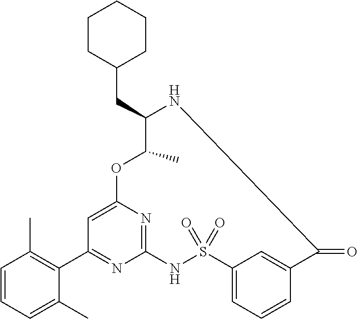 [SMILES: Cc1cccc(C)c1-c1cc2nc(n1)NS(=O)(=O)c1cccc(c1)C(=O)N[C@H](CC1CCCCC1)[C@H](C)O2] | +++ | +++ | +++ | +++ | | |
| 315 | 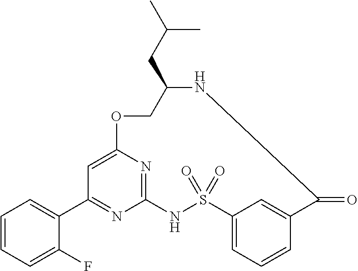 [SMILES: CC(C)C[C@@H]1COc2cc(-c3ccccc3F)nc(n2)NS(=O)(=O)c2cccc(c2)C(=O)N1] | +++ | +++ | ++ | +++ | | |
| 98 | 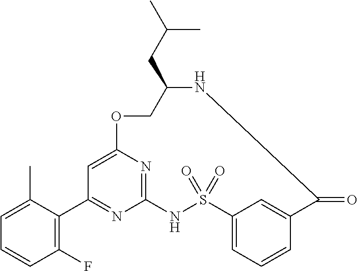 [SMILES: Cc1cccc(F)c1-c1cc2nc(n1)NS(=O)(=O)c1cccc(c1)C(=O)N[C@H](CC(C)C)CO2] | +++ | +++ | +++ | +++ | | |
| 75 | 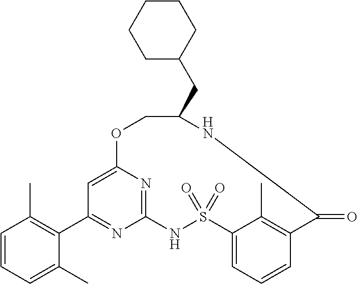 [SMILES: Cc1cccc(C)c1-c1cc2nc(n1)NS(=O)(=O)c1cccc(c1C)C(=O)N[C@H](CC1CCCCC1)CO2] | +++ | +++ | +++ | +++ | | |
| 493 | 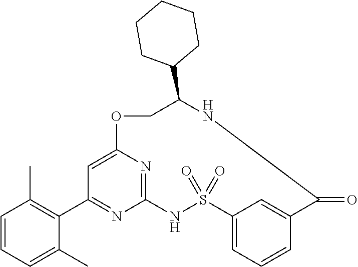 [SMILES: Cc1cccc(C)c1-c1cc2nc(n1)NS(=O)(=O)c1cccc(c1)C(=O)N[C@H](C1CCCCC1)CO2] | +++ | +++ | +++ | +++ | | |
| 411 | 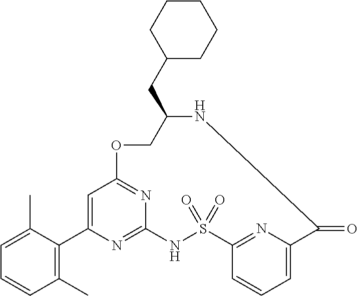 [SMILES: Cc1cccc(C)c1-c1cc2nc(n1)NS(=O)(=O)c1cccc(n1)C(=O)N[C@H](CC1CCCCC1)CO2] | +++ | +++ | +++ | +++ | | |
| 142 | 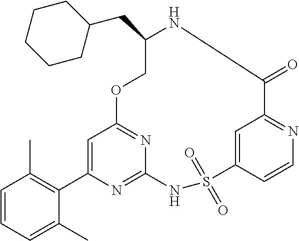 [SMILES: Cc1cccc(C)c1-c1cc2nc(n1)NS(=O)(=O)c1ccnc(c1)C(=O)N[C@H](CC1CCCCC1)CO2] | +++ | +++ | ++ | +++ | +++ | ++ |
| 287 | 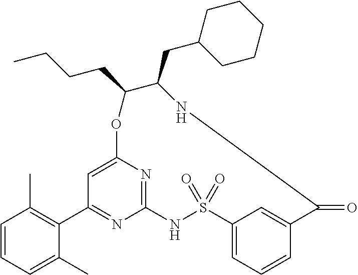 [SMILES: CCCC[C@@H]1Oc2cc(-c3c(C)cccc3C)nc(n2)NS(=O)(=O)c2cccc(c2)C(=O)N[C@@H]1CC1CCCCC1] | +++ | +++ | +++ | +++ | | |
| 361 | 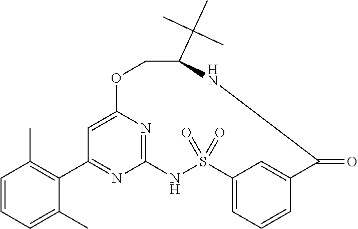 [SMILES: Cc1cccc(C)c1-c1cc2nc(n1)NS(=O)(=O)c1cccc(c1)C(=O)N[C@H](C(C)(C)C)CO2] | +++ | +++ | +++ | +++ | | |
| 360 | 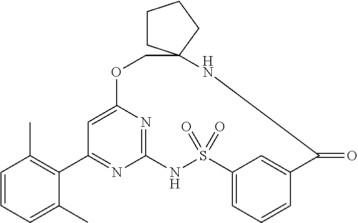 [SMILES: Cc1cccc(C)c1-c1cc2nc(n1)NS(=O)(=O)c1cccc(c1)C(=O)NC1(CCCC1)CO2] | | | +++ | +++ | | |
| 305 | 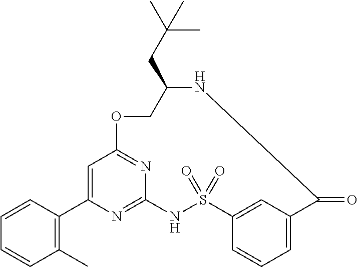 [SMILES: Cc1ccccc1-c1cc2nc(n1)NS(=O)(=O)c1cccc(c1)C(=O)N[C@H](CC(C)(C)C)CO2] | +++ | +++ | +++ | +++ | | |
| 314 | 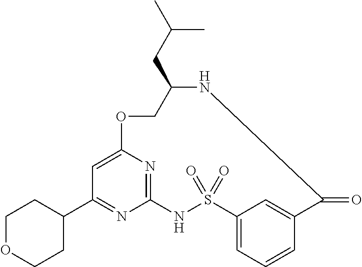 [SMILES: CC(C)C[C@@H]1COc2cc(C3CCOCC3)nc(n2)NS(=O)(=O)c2cccc(c2)C(=O)N1] | | | + | + | | |
| 313 | 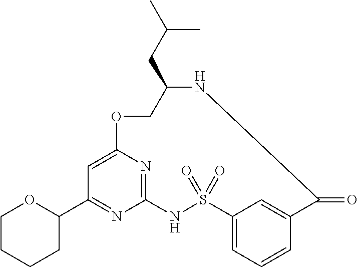 [SMILES: CC(C)C[C@@H]1COc2cc(C3CCCCO3)nc(n2)NS(=O)(=O)c2cccc(c2)C(=O)N1] | | | + | ++ | | |
| 312 | 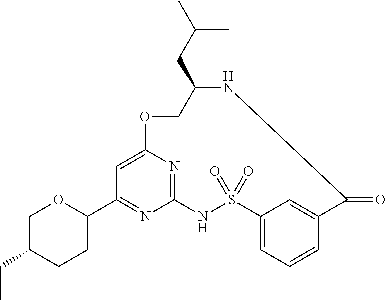 [SMILES: CC[C@H]1CCC(c2cc3nc(n2)NS(=O)(=O)c2cccc(c2)C(=O)N[C@H](CC(C)C)CO3)OC1] | | | +++ | ++ | | |
| 311 | 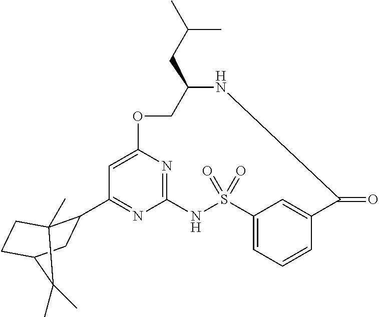 [SMILES: CC(C)C[C@@H]1COc2cc(C3CC4CCC3(C)C4(C)C)nc(n2)NS(=O)(=O)c2cccc(c2)C(=O)N1] | | | +++ | ++ | | |
| 310 | 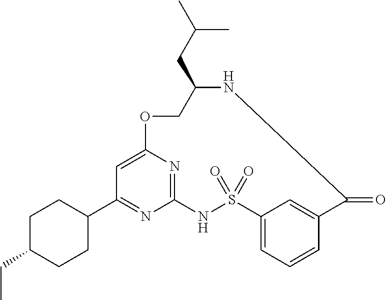 | | | +++ | ++ | | |
| 309 | 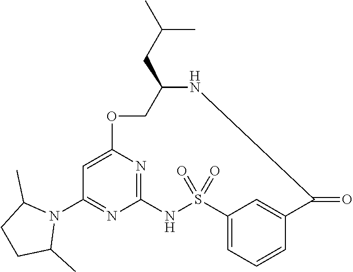 [SMILES: CC(C)C[C@@H]1COc2cc(N3C(C)CCC3C)nc(n2)NS(=O)(=O)c2cccc(c2)C(=O)N1] | | | ND | + | | |
| 308 | 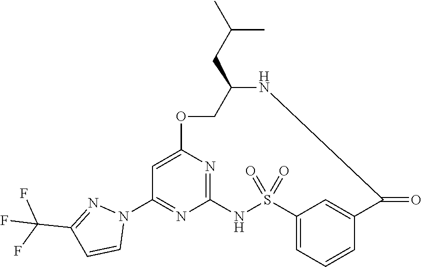 [SMILES: CC(C)C[C@@H]1COc2cc(-n3ccc(C(F)(F)F)n3)nc(n2)NS(=O)(=O)c2cccc(c2)C(=O)N1] | | | ND | + | | |
| 307 | 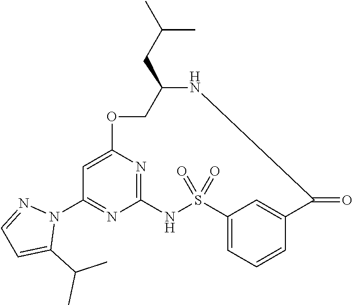 [SMILES: CC(C)C[C@@H]1COc2cc(-n3nccc3C(C)C)nc(n2)NS(=O)(=O)c2cccc(c2)C(=O)N1] | | | ND | + | | |
| 109 | 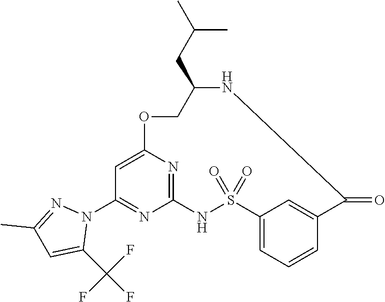 [SMILES: Cc1cc(C(F)(F)F)n(-c2cc3nc(n2)NS(=O)(=O)c2cccc(c2)C(=O)N[C@H](CC(C)C)CO3)n1] | | | +++ | + | | |
| 306 | 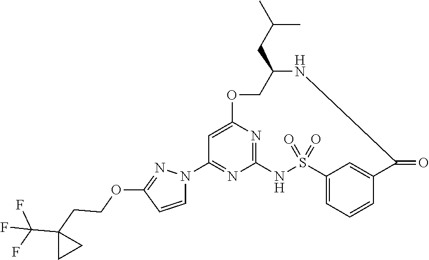 [SMILES: CC(C)C[C@@H]1COc2cc(-n3ccc(OCCC4(C(F)(F)F)CC4)n3)nc(n2)NS(=O)(=O)c2cccc(c2)C(=O)N1] | | | ND | + | | |
| 99 | 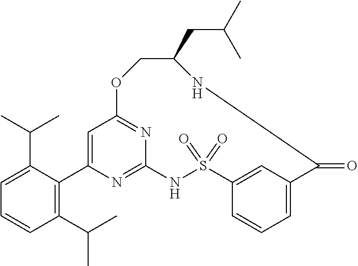 [SMILES: CC(C)C[C@@H]1COc2cc(-c3c(C(C)C)cccc3C(C)C)nc(n2)NS(=O)(=O)c2cccc(c2)C(=O)N1] | | | +++ | +++ | | |
| 159 | 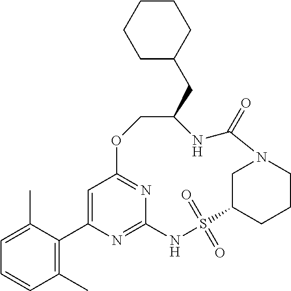 [SMILES: Cc1cccc(C)c1-c1cc2nc(n1)NS(=O)(=O)[C@H]1CCCN(C1)C(=O)N[C@H](CC1CCCCC1)CO2] | | | ND | + | | |
| 190 | 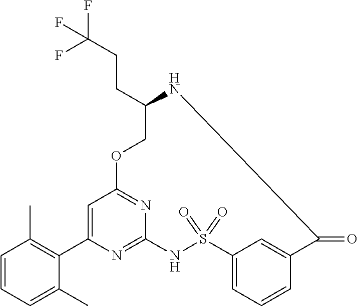 [SMILES: Cc1cccc(C)c1-c1cc2nc(n1)NS(=O)(=O)c1cccc(c1)C(=O)N[C@H](CCC(F)(F)F)CO2] | | | +++ | +++ | | |
| 189 | 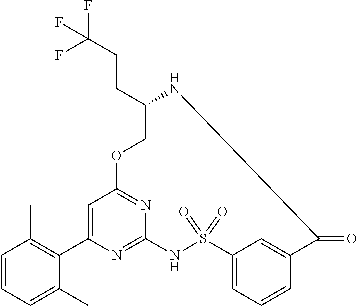 [SMILES: Cc1cccc(C)c1-c1cc2nc(n1)NS(=O)(=O)c1cccc(c1)C(=O)N[C@@H](CCC(F)(F)F)CO2] | | | ++ | + | | |
| 188 | 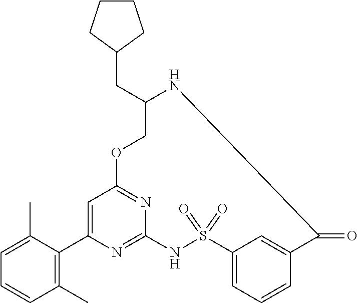 [SMILES: Cc1cccc(C)c1-c1cc2nc(n1)NS(=O)(=O)c1cccc(c1)C(=O)NC(CC1CCCC1)CO2] | | | +++ | +++ | | |
| 187 | 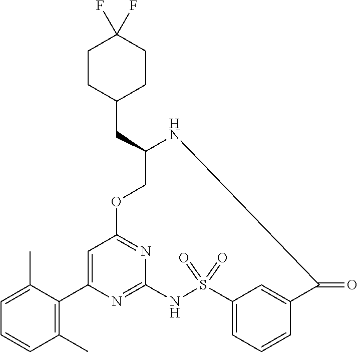 [SMILES: Cc1cccc(C)c1-c1cc2nc(n1)NS(=O)(=O)c1cccc(c1)C(=O)N[C@H](CC1CCC(F)(F)CC1)CO2] | | | +++ | +++ | | |
| 186 | 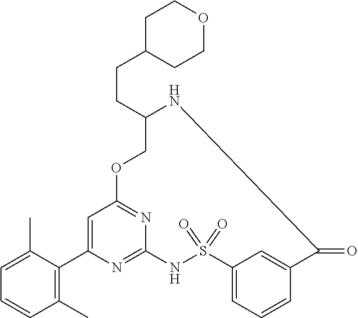 [SMILES: Cc1cccc(C)c1-c1cc2nc(n1)NS(=O)(=O)c1cccc(c1)C(=O)NC(CCC1CCOCC1)CO2] | | | + | +++ | | |
| 239 | 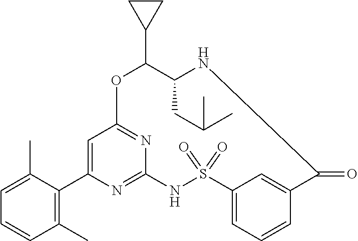 [SMILES: Cc1cccc(C)c1-c1cc2nc(n1)NS(=O)(=O)c1cccc(c1)C(=O)N[C@H](CC(C)C)C(C1CC1)O2] | +++ | +++ | +++ | +++ | | |
| 238 | 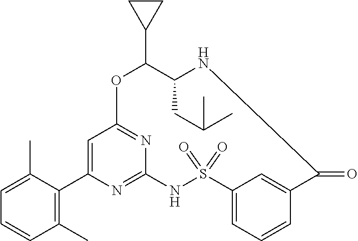 [SMILES: Cc1cccc(C)c1-c1cc2nc(n1)NS(=O)(=O)c1cccc(c1)C(=O)N[C@H](CC(C)C)C(C1CC1)O2] | | | ++ | +++ | | |
| 141 | 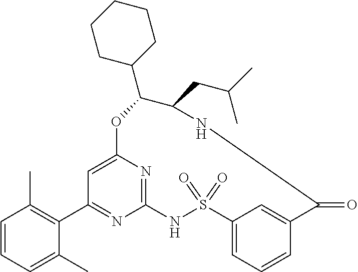 [SMILES: Cc1cccc(C)c1-c1cc2nc(n1)NS(=O)(=O)c1cccc(c1)C(=O)N[C@H](CC(C)C)[C@@H](C1CCCCC1)O2] | | | | | | |
| 140 | 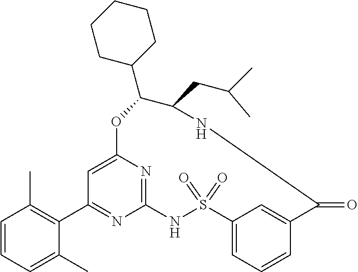 [SMILES: Cc1cccc(C)c1-c1cc2nc(n1)NS(=O)(=O)c1cccc(c1)C(=O)N[C@H](CC(C)C)[C@@H](C1CCCCC1)O2] | +++ | +++ | +++ | +++ | | |
| 69 | 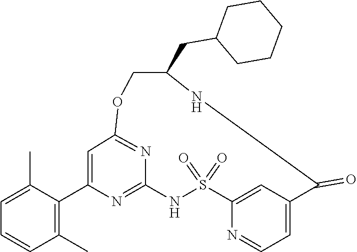 [SMILES: Cc1cccc(C)c1-c1cc2nc(n1)NS(=O)(=O)c1cc(ccn1)C(=O)N[C@H](CC1CCCCC1)CO2] | | | +++ | +++ | | |
| 47 | 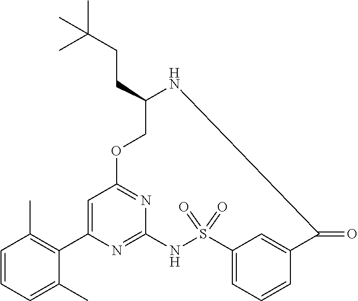 [SMILES: Cc1cccc(C)c1-c1cc2nc(n1)NS(=O)(=O)c1cccc(c1)C(=O)N[C@H](CCC(C)(C)C)CO2] | +++ | +++ | +++ | +++ | | |
| 48 | 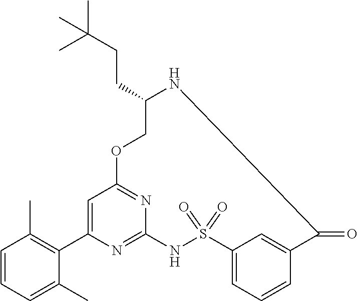 [SMILES: Cc1cccc(C)c1-c1cc2nc(n1)NS(=O)(=O)c1cccc(c1)C(=O)N[C@@H](CCC(C)(C)C)CO2] | | | +++ | + | | |
| 494 | 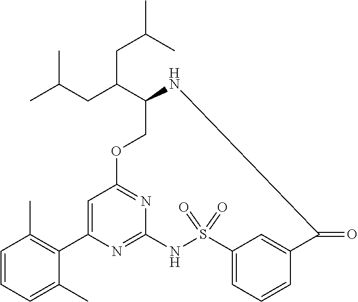 [SMILES: Cc1cccc(C)c1-c1cc2nc(n1)NS(=O)(=O)c1cccc(c1)C(=O)N[C@H](C(CC(C)C)CC(C)C)CO2] | +++ | +++ | +++ | +++ | | |
| 304 | 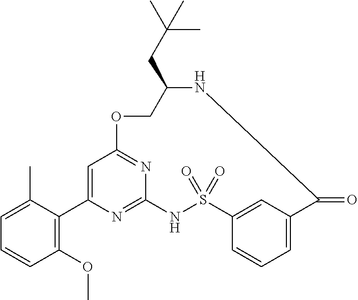 [SMILES: COc1cccc(C)c1-c1cc2nc(n1)NS(=O)(=O)c1cccc(c1)C(=O)N[C@H](CC(C)(C)C)CO2] | | | ++ | +++ | | |
| 303 | 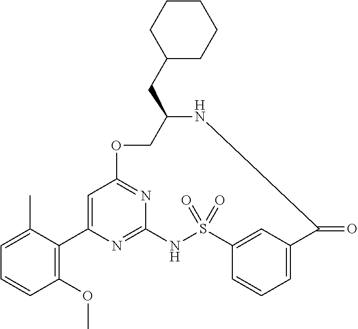 [SMILES: COc1cccc(C)c1-c1cc2nc(n1)NS(=O)(=O)c1cccc(c1)C(=O)N[C@H](CC1CCCCC1)CO2] | | | +++ | + | | |
| 66 | 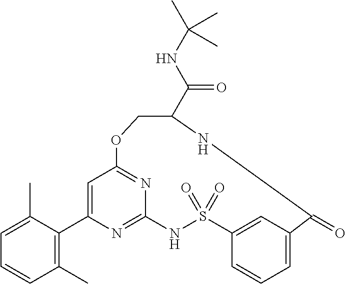 [SMILES: Cc1cccc(C)c1-c1cc2nc(n1)NS(=O)(=O)c1cccc(c1)C(=O)NC(C(=O)NC(C)(C)C)CO2] | | | ND | + | | |
| 237 | 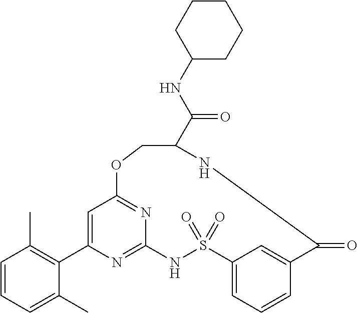 [SMILES: Cc1cccc(C)c1-c1cc2nc(n1)NS(=O)(=O)c1cccc(c1)C(=O)NC(C(=O)NC1CCCCC1)CO2] | | | ND | + | | |
| 236 | 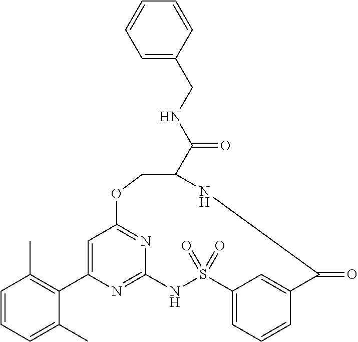 [SMILES: Cc1cccc(C)c1-c1cc2nc(n1)NS(=O)(=O)c1cccc(c1)C(=O)NC(C(=O)NCc1ccccc1)CO2] | | | ND | + | | |
| 359 | 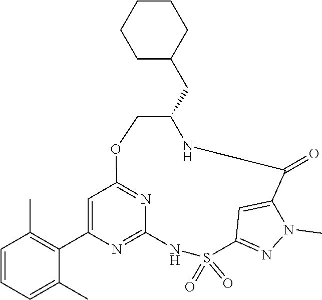 [SMILES: Cc1cccc(C)c1-c1cc2nc(n1)NS(=O)(=O)c1cc(n(C)n1)C(=O)N[C@@H](CC1CCCCC1)CO2] | | | ND | + | | |
| 302 | 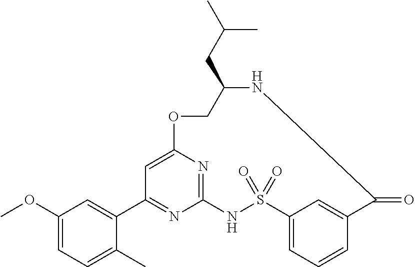 [SMILES: COc1ccc(C)c(-c2cc3nc(n2)NS(=O)(=O)c2cccc(c2)C(=O)N[C@H](CC(C)C)CO3)c1] | | | ++ | +++ | | |
| 235 | 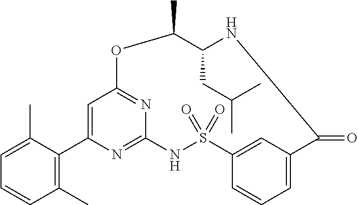 [SMILES: Cc1cccc(C)c1-c1cc2nc(n1)NS(=O)(=O)c1cccc(c1)C(=O)N[C@H](CC(C)C)[C@H](C)O2] | | | +++ | +++ | | |
| 100 | 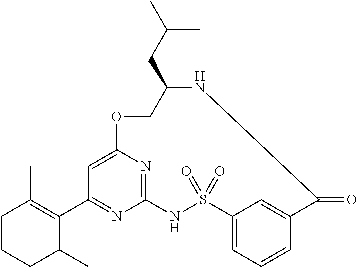 [SMILES: CC1=C(c2cc3nc(n2)NS(=O)(=O)c2cccc(c2)C(=O)N[C@H](CC(C)C)CO3)C(C)CCC1] | +++ | +++ | ++ | +++ | | |
| 410 | 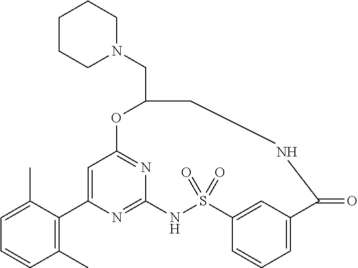 [SMILES: Cc1cccc(C)c1-c1cc2nc(n1)NS(=O)(=O)c1cccc(c1)C(=O)NCC(CN1CCCCC1)O2] | | | + | ++ | | |
| 3 | 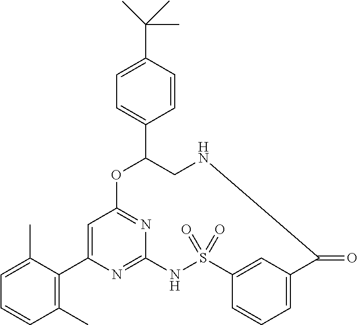 [SMILES: Cc1cccc(C)c1-c1cc2nc(n1)NS(=O)(=O)c1cccc(c1)C(=O)NCC(c1ccc(C(C)(C)C)cc1)O2] | | | +++ | +++ | | |
| 301 | 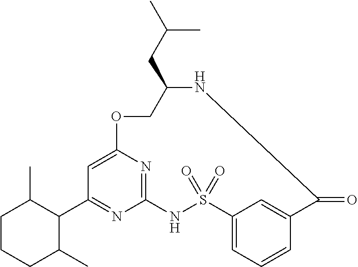 [SMILES: CC(C)C[C@@H]1COc2cc(C3C(C)CCCC3C)nc(n2)NS(=O)(=O)c2cccc(c2)C(=O)N1] | | | + | ++ | | |
| 300 | 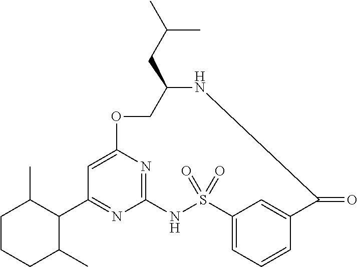 [SMILES: CC(C)C[C@@H]1COc2cc(C3C(C)CCCC3C)nc(n2)NS(=O)(=O)c2cccc(c2)C(=O)N1] | | | +++ | ++ | | |
| 286 | 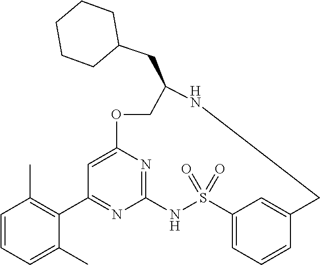 [SMILES: Cc1cccc(C)c1-c1cc2nc(n1)NS(=O)(=O)c1cccc(c1)CN[C@H](CC1CCCCC1)CO2] | | | + | + | | |
| 234 | 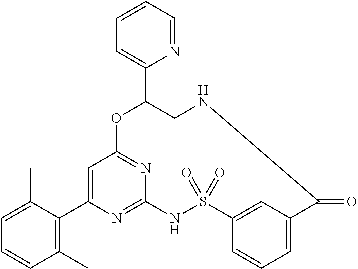 [SMILES: Cc1cccc(C)c1-c1cc2nc(n1)NS(=O)(=O)c1cccc(c1)C(=O)NCC(c1ccccn1)O2] | | | ND | + | | |
| 409 | 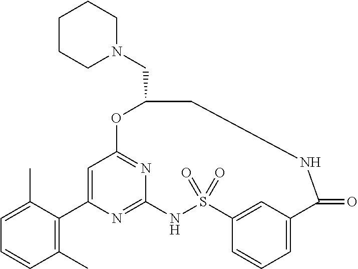 [SMILES: Cc1cccc(C)c1-c1cc2nc(n1)NS(=O)(=O)c1cccc(c1)C(=O)NC[C@@H](CN1CCCCC1)O2] | | | ND | + | | |
| 408 | 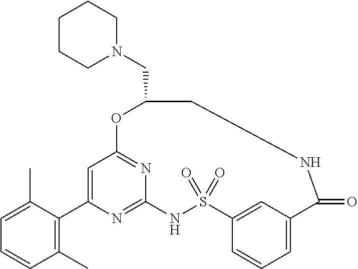 [SMILES: Cc1cccc(C)c1-c1cc2nc(n1)NS(=O)(=O)c1cccc(c1)C(=O)NC[C@@H](CN1CCCCC1)O2] | | | ND | ++ | | |
| 281 | 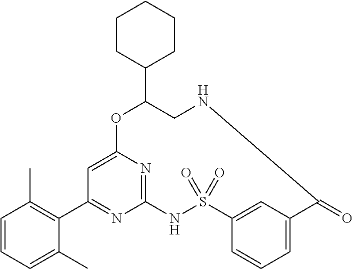 [SMILES: Cc1cccc(C)c1-c1cc2nc(n1)NS(=O)(=O)c1cccc(c1)C(=O)NCC(C1CCCCC1)O2] | | | +++ | +++ | | |
| 136 | 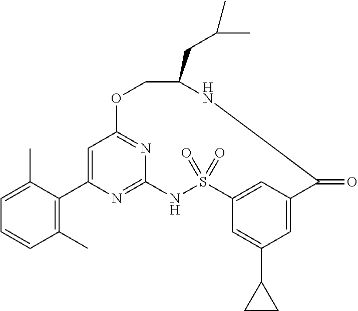 [SMILES: Cc1cccc(C)c1-c1cc2nc(n1)NS(=O)(=O)c1cc(cc(C3CC3)c1)C(=O)N[C@H](CC(C)C)CO2] | | | ND | + | | |
| 135 | 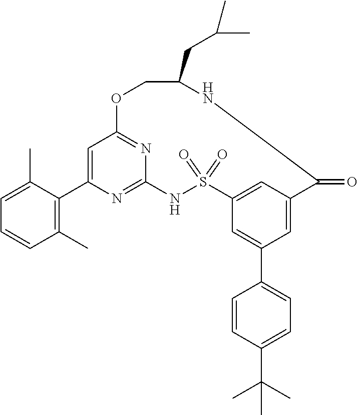 [SMILES: Cc1cccc(C)c1-c1cc2nc(n1)NS(=O)(=O)c1cc(cc(-c3ccc(C(C)(C)C)cc3)c1)C(=O)N[C@H](CC(C)C)CO2] | | | ND | + | | |
| 139 | 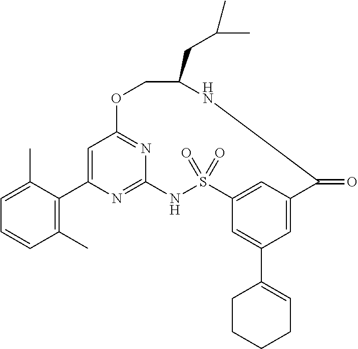 [SMILES: Cc1cccc(C)c1-c1cc2nc(n1)NS(=O)(=O)c1cc(cc(C3=CCCCC3)c1)C(=O)N[C@H](CC(C)C)CO2] | | | ND | + | | |
| 138 | 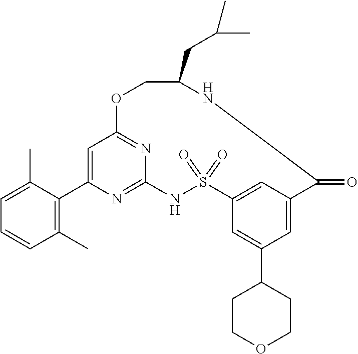 [SMILES: Cc1cccc(C)c1-c1cc2nc(n1)NS(=O)(=O)c1cc(cc(C3CCOCC3)c1)C(=O)N[C@H](CC(C)C)CO2] | | | ND | + | | |
| 137 | 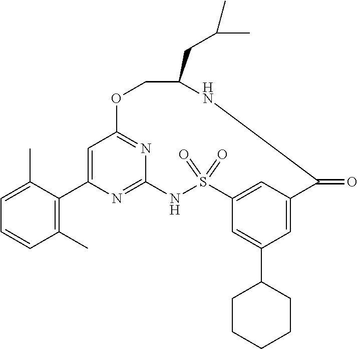 [SMILES: Cc1cccc(C)c1-c1cc2nc(n1)NS(=O)(=O)c1cc(cc(C3CCCCC3)c1)C(=O)N[C@H](CC(C)C)CO2] | | | ND | + | | |
| 386 | 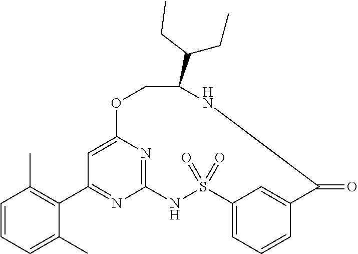 [SMILES: CCC(CC)[C@@H]1COc2cc(-c3c(C)cccc3C)nc(n2)NS(=O)(=O)c2cccc(c2)C(=O)N1] | | | +++ | +++ | | |
| 4 | 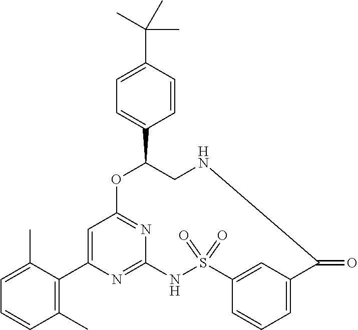 [SMILES: Cc1cccc(C)c1-c1cc2nc(n1)NS(=O)(=O)c1cccc(c1)C(=O)NC[C@H](c1ccc(C(C)(C)C)cc1)O2] | | | + | + | ND | + |
| 5 | 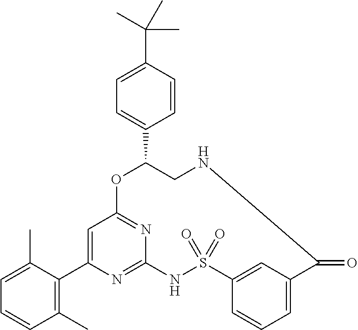 [SMILES: Cc1cccc(C)c1-c1cc2nc(n1)NS(=O)(=O)c1cccc(c1)C(=O)NC[C@@H](c1ccc(C(C)(C)C)cc1)O2] | +++ | +++ | +++ | +++ | +++ | +++ |
| 290 | 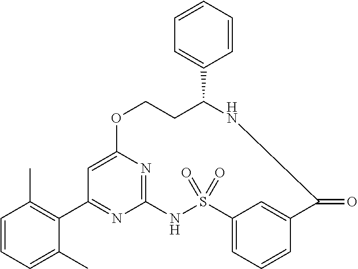 [SMILES: Cc1cccc(C)c1-c1cc2nc(n1)NS(=O)(=O)c1cccc(c1)C(=O)N[C@@H](c1ccccc1)CCO2] | | | ++ | ++ | | |
| 233 | 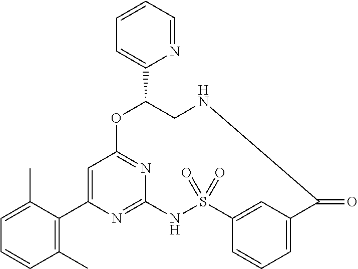 [SMILES: Cc1cccc(C)c1-c1cc2nc(n1)NS(=O)(=O)c1cccc(c1)C(=O)NC[C@@H](c1ccccn1)O2] | | | ND | + | | |
| 232 | 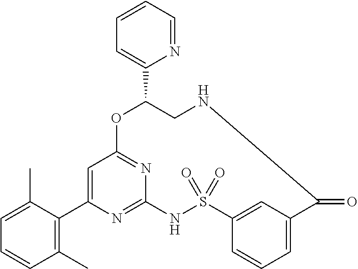 [SMILES: Cc1cccc(C)c1-c1cc2nc(n1)NS(=O)(=O)c1cccc(c1)C(=O)NC[C@@H](c1ccccn1)O2] | | | ND | + | | |
| 30 | 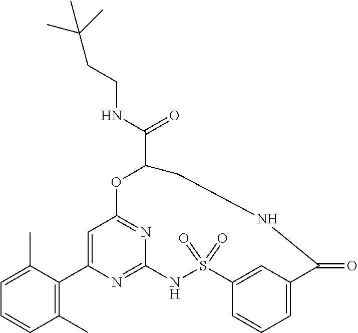 [SMILES: Cc1cccc(C)c1-c1cc2nc(n1)NS(=O)(=O)c1cccc(c1)C(=O)NCC(C(=O)NCCC(C)(C)C)O2] | | | + | ++ | | |
| 231 | 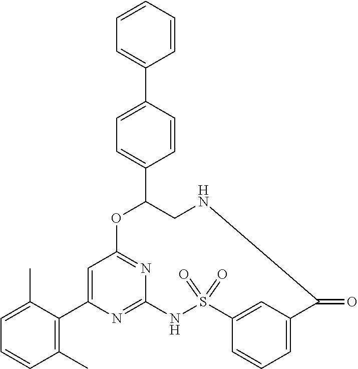 [SMILES: Cc1cccc(C)c1-c1cc2nc(n1)NS(=O)(=O)c1cccc(c1)C(=O)NCC(c1ccc(-c3ccccc3)cc1)O2] | | | +++ | ++ | | |
| 18 | 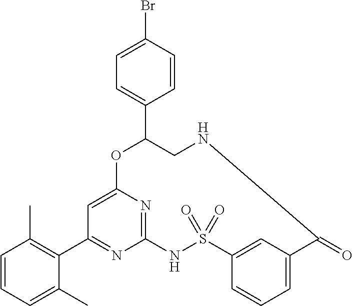 [SMILES: Cc1cccc(C)c1-c1cc2nc(n1)NS(=O)(=O)c1cccc(c1)C(=O)NCC(c1ccc(Br)cc1)O2] | | | +++ | +++ | | |
| 253 | 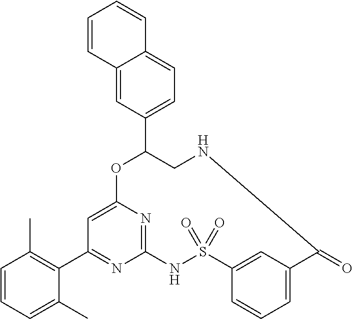 [SMILES: Cc1cccc(C)c1-c1cc2nc(n1)NS(=O)(=O)c1cccc(c1)C(=O)NCC(c1ccc3ccccc3c1)O2] | | | +++ | +++ | | |
| 296 | 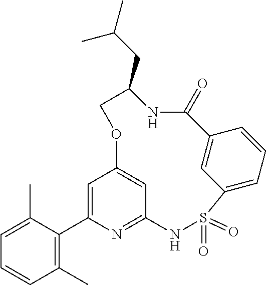 [SMILES: Cc1cccc(C)c1-c1cc2cc(n1)NS(=O)(=O)c1cccc(c1)C(=O)N[C@H](CC(C)C)CO2] | | | +++ | +++ | | |
| 73 | 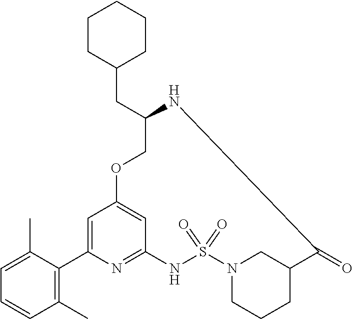 [SMILES: Cc1cccc(C)c1-c1cc2cc(n1)NS(=O)(=O)N1CCCC(C1)C(=O)N[C@H](CC1CCCCC1)CO2] | ND | ++ | ND | + | | |
| 31 | 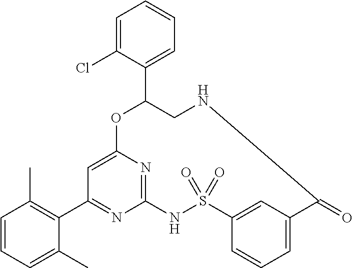 [SMILES: Cc1cccc(C)c1-c1cc2nc(n1)NS(=O)(=O)c1cccc(c1)C(=O)NCC(c1ccccc1Cl)O2] | | | +++ | +++ | | |
| 252 | 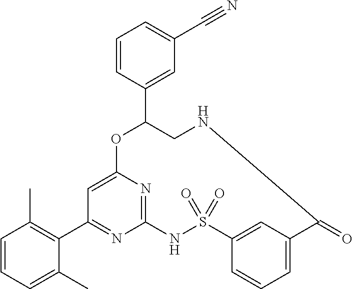 [SMILES: Cc1cccc(C)c1-c1cc2nc(n1)NS(=O)(=O)c1cccc(c1)C(=O)NCC(c1cccc(C#N)c1)O2] | | | + | ++ | | |
| 32 | 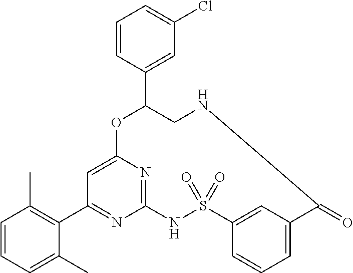 [SMILES: Cc1cccc(C)c1-c1cc2nc(n1)NS(=O)(=O)c1cccc(c1)C(=O)NCC(c1cccc(Cl)c1)O2] | | | +++ | +++ | | |
| 251 | 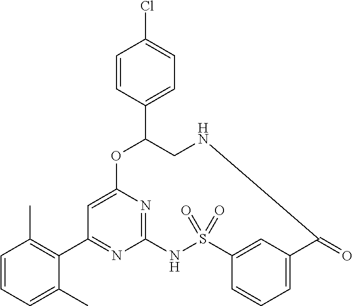 [SMILES: Cc1cccc(C)c1-c1cc2nc(n1)NS(=O)(=O)c1cccc(c1)C(=O)NCC(c1ccc(Cl)cc1)O2] | | | +++ | +++ | | |
| 250 | 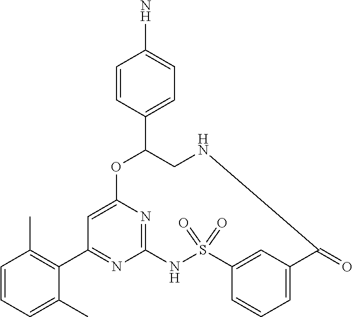 | | | +++ | +++ | | |
| 249 | 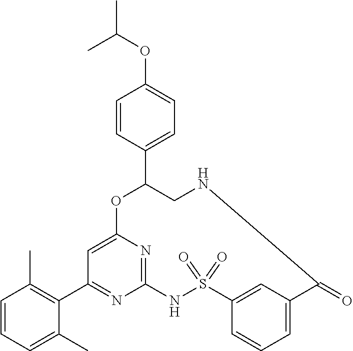 [SMILES: Cc1cccc(C)c1-c1cc2nc(n1)NS(=O)(=O)c1cccc(c1)C(=O)NCC(c1ccc(OC(C)C)cc1)O2] | | | +++ | ++ | | |
| 407 | 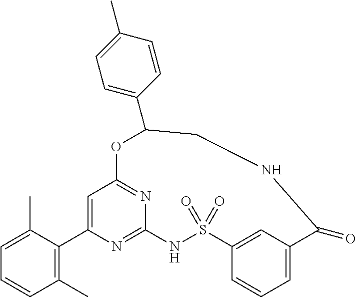 [SMILES: Cc1ccc(C2CNC(=O)c3cccc(c3)S(=O)(=O)Nc3nc(cc(-c4c(C)cccc4C)n3)O2)cc1] | | | +++ | +++ | | |
| 248 | 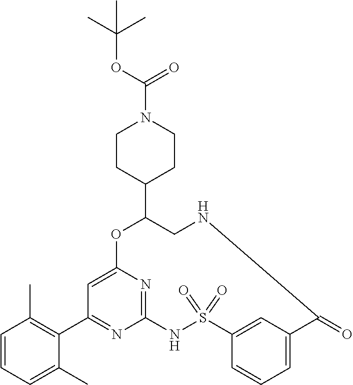 [SMILES: Cc1cccc(C)c1-c1cc2nc(n1)NS(=O)(=O)c1cccc(c1)C(=O)NCC(C1CCN(C(=O)OC(C)(C)C)CC1)O2] | | | +++ | +++ | | |
| 247 | 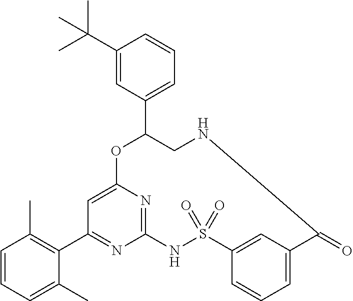 [SMILES: Cc1cccc(C)c1-c1cc2nc(n1)NS(=O)(=O)c1cccc(c1)C(=O)NCC(c1cccc(C(C)(C)C)c1)O2] | | | +++ | +++ | | |
| 246 | 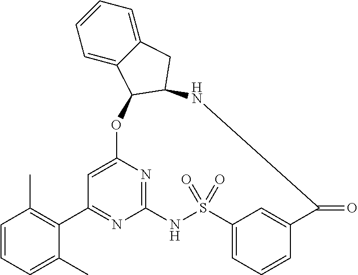 [SMILES: Cc1cccc(C)c1-c1cc2nc(n1)NS(=O)(=O)c1cccc(c1)C(=O)N[C@@H]1Cc3ccccc3[C@@H]1O2] | | | +++ | +++ | | |
| 157 | 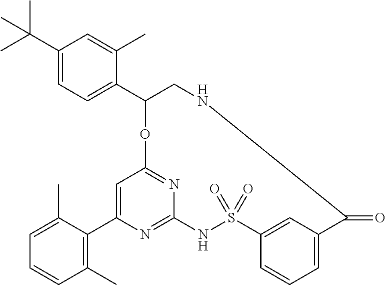 [SMILES: Cc1cc(C(C)(C)C)ccc1C1CNC(=O)c2cccc(c2)S(=O)(=O)Nc2nc(cc(-c3c(C)cccc3C)n2)O1] | | | +++ | +++ | | |
| 158 | 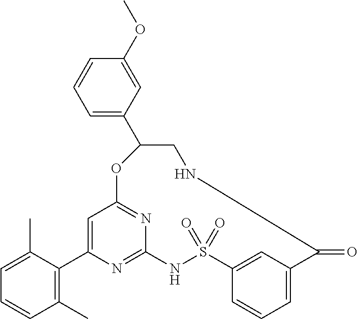 [SMILES: COc1cccc(C2CNC(=O)c3cccc(c3)S(=O)(=O)Nc3nc(cc(-c4c(C)cccc4C)n3)O2)c1] | | | +++ | +++ | | |
| 404 | 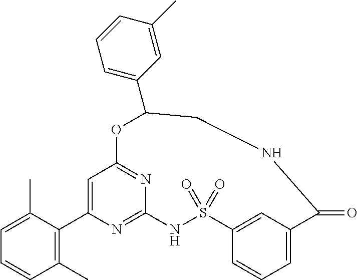 [SMILES: Cc1cccc(C2CNC(=O)c3cccc(c3)S(=O)(=O)Nc3nc(cc(-c4c(C)cccc4C)n3)O2)c1] | | | +++ | +++ | | |
| 279 | 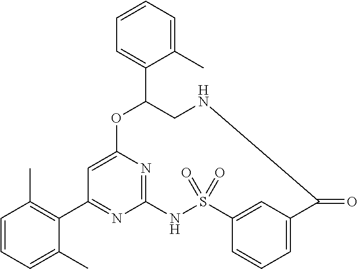 [SMILES: Cc1ccccc1C1CNC(=O)c2cccc(c2)S(=O)(=O)Nc2nc(cc(-c3c(C)cccc3C)n2)O1] | | | +++ | +++ | | |
| 280 | 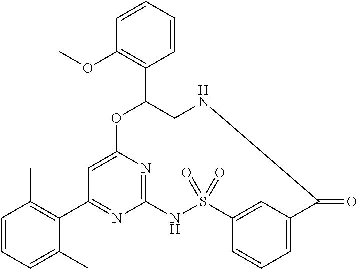 [SMILES: COc1ccccc1C1CNC(=O)c2cccc(c2)S(=O)(=O)Nc2nc(cc(-c3c(C)cccc3C)n2)O1] | | | +++ | +++ | | |
| 285 | 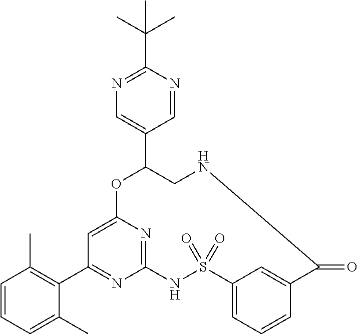 [SMILES: Cc1cccc(C)c1-c1cc2nc(n1)NS(=O)(=O)c1cccc(c1)C(=O)NCC(c1cnc(C(C)(C)C)nc1)O2] | | | ND | + | | |
| 403 | 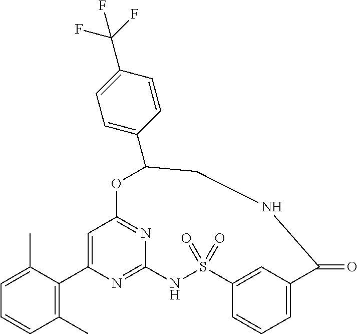 [SMILES: Cc1cccc(C)c1-c1cc2nc(n1)NS(=O)(=O)c1cccc(c1)C(=O)NCC(c1ccc(C(F)(F)F)cc1)O2] | | | +++ | ++ | | |
| 406 | 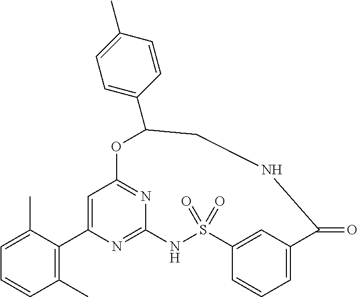 [SMILES: Cc1ccc(C2CNC(=O)c3cccc(c3)S(=O)(=O)Nc3nc(cc(-c4c(C)cccc4C)n3)O2)cc1] | | | ND | + | | |
| 405 | 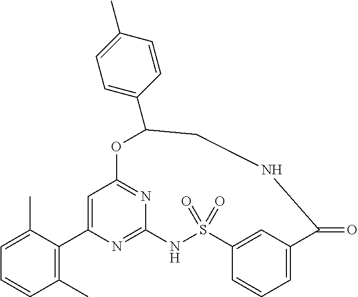 [SMILES: Cc1ccc(C2CNC(=O)c3cccc(c3)S(=O)(=O)Nc3nc(cc(-c4c(C)cccc4C)n3)O2)cc1] | | | +++ | ++ | +++ | ++ |
| 255 | 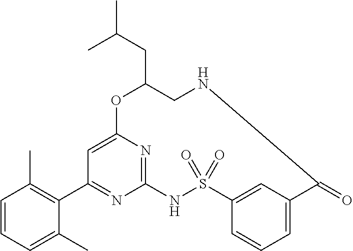 [SMILES: Cc1cccc(C)c1-c1cc2nc(n1)NS(=O)(=O)c1cccc(c1)C(=O)NCC(CC(C)C)O2] | | | ++ | ++ | | |
| 254 | 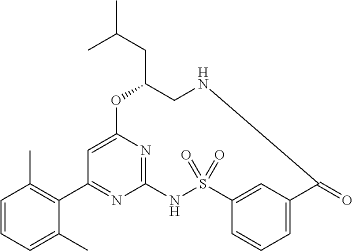 [SMILES: Cc1cccc(C)c1-c1cc2nc(n1)NS(=O)(=O)c1cccc(c1)C(=O)NC[C@@H](CC(C)C)O2] | | | ND | + | | |
| 245 | 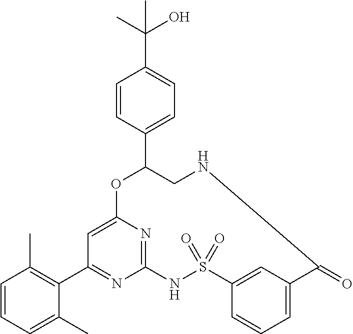 [SMILES: Cc1cccc(C)c1-c1cc2nc(n1)NS(=O)(=O)c1cccc(c1)C(=O)NCC(c1ccc(C(C)(C)O)cc1)O2] | | | + | ++ | | |
| 402 | 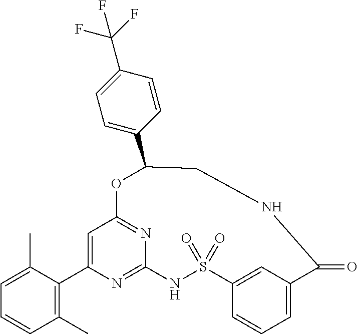 [SMILES: Cc1cccc(C)c1-c1cc2nc(n1)NS(=O)(=O)c1cccc(c1)C(=O)NC[C@H](c1ccc(C(F)(F)F)cc1)O2] | | | + | + | | |
| 401 | 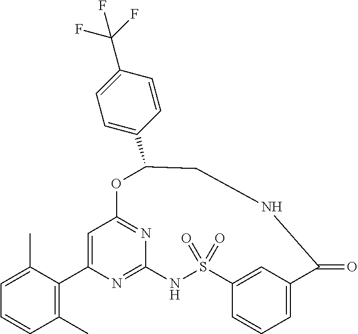 [SMILES: Cc1cccc(C)c1-c1cc2nc(n1)NS(=O)(=O)c1cccc(c1)C(=O)NC[C@@H](c1ccc(C(F)(F)F)cc1)O2] | +++ | +++ | +++ | +++ | | |
| 6 | 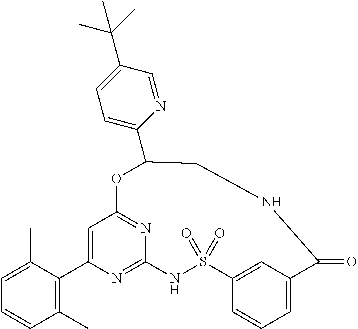 [SMILES: Cc1cccc(C)c1-c1cc2nc(n1)NS(=O)(=O)c1cccc(c1)C(=O)NCC(c1ccc(C(C)(C)C)cn1)O2] | | | +++ | ++ | | |
| 278 | 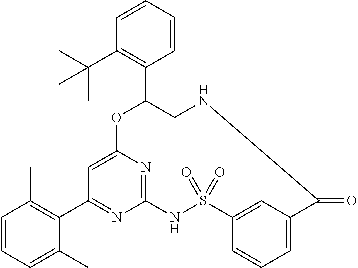 [SMILES: Cc1cccc(C)c1-c1cc2nc(n1)NS(=O)(=O)c1cccc(c1)C(=O)NCC(c1ccccc1C(C)(C)C)O2] | | | +++ | ++ | | |
| 244 | 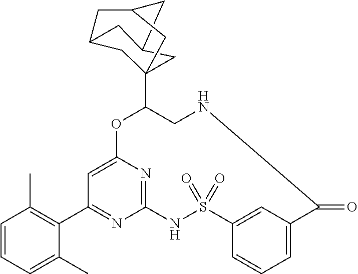 [SMILES: Cc1cccc(C)c1-c1cc2nc(n1)NS(=O)(=O)c1cccc(c1)C(=O)NCC(C13CC4CC(CC(C4)C1)C3)O2] | | | +++ | ++ | | |
| 38 | 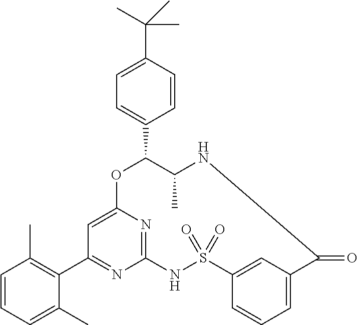 [SMILES: Cc1cccc(C)c1-c1cc2nc(n1)NS(=O)(=O)c1cccc(c1)C(=O)N[C@H](C)[C@@H](c1ccc(C(C)(C)C)cc1)O2] | +++ | +++ | +++ | +++ | +++ | +++ |
| 134 | 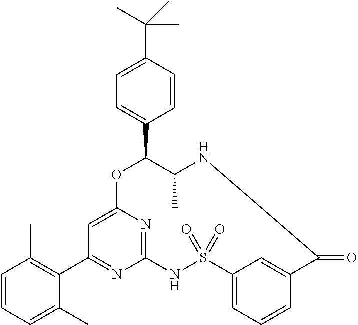 [SMILES: Cc1cccc(C)c1-c1cc2nc(n1)NS(=O)(=O)c1cccc(c1)C(=O)N[C@H](C)[C@H](c1ccc(C(C)(C)C)cc1)O2] | | | +++ | + | | |
| 7 | 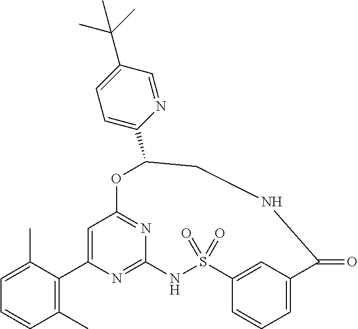 [SMILES: Cc1cccc(C)c1-c1cc2nc(n1)NS(=O)(=O)c1cccc(c1)C(=O)NC[C@@H](c1ccc(C(C)(C)C)cn1)O2] | +++ | +++ | +++ | +++ | | |
| 8 | 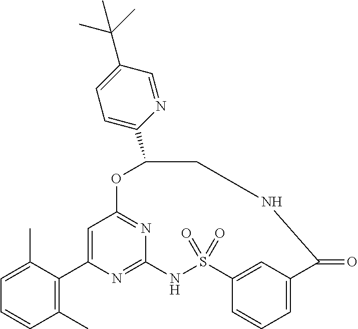 [SMILES: Cc1cccc(C)c1-c1cc2nc(n1)NS(=O)(=O)c1cccc(c1)C(=O)NC[C@@H](c1ccc(C(C)(C)C)cn1)O2] | | | ND | + | | |
| 19 | 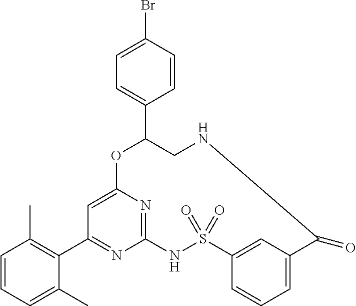 [SMILES: Cc1cccc(C)c1-c1cc2nc(n1)NS(=O)(=O)c1cccc(c1)C(=O)NCC(c1ccc(Br)cc1)O2] | | | ND | + | | |
| 20 | 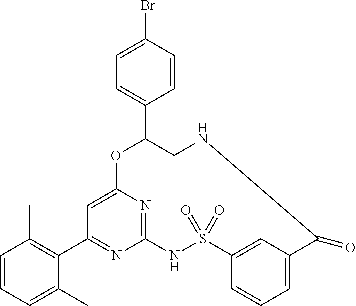 [SMILES: Cc1cccc(C)c1-c1cc2nc(n1)NS(=O)(=O)c1cccc(c1)C(=O)NCC(c1ccc(Br)cc1)O2] | | | +++ | ++ | | |
| 33 | 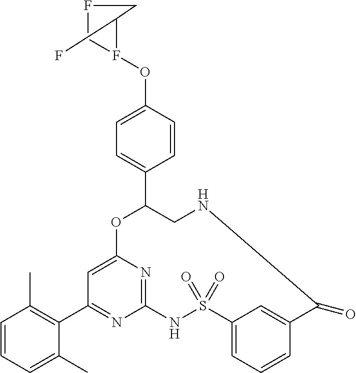 | | | +++ | +++ | | |
| 34 | 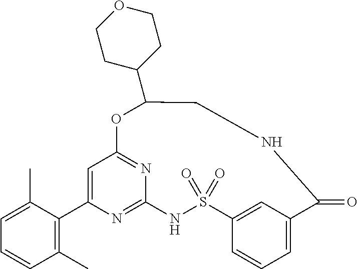 [SMILES: Cc1cccc(C)c1-c1cc2nc(n1)NS(=O)(=O)c1cccc(c1)C(=O)NCC(C1CCOCC1)O2] | | | ND | + | | |
| 21 | 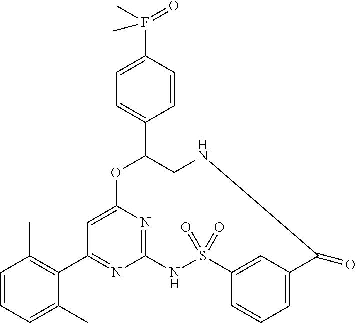 | | | ND | + | | |
| 277 | 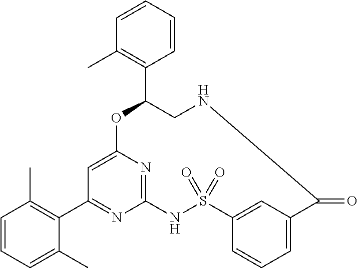 [SMILES: Cc1ccccc1[C@H]1CNC(=O)c2cccc(c2)S(=O)(=O)Nc2nc(cc(-c3c(C)cccc3C)n2)O1] | | | ND | + | | |
| 276 | 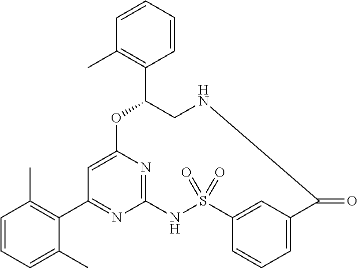 [SMILES: Cc1ccccc1[C@@H]1CNC(=O)c2cccc(c2)S(=O)(=O)Nc2nc(cc(-c3c(C)cccc3C)n2)O1] | | | +++ | ++ | | |
| 156 | 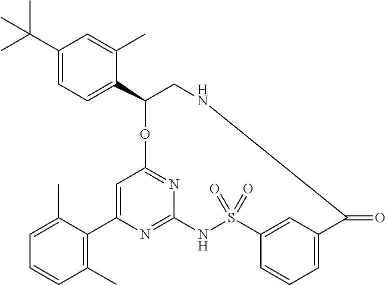 [SMILES: Cc1cc(C(C)(C)C)ccc1[C@H]1CNC(=O)c2cccc(c2)S(=O)(=O)Nc2nc(cc(-c3c(C)cccc3C)n2)O1] | | | ND | + | | |
| 155 | 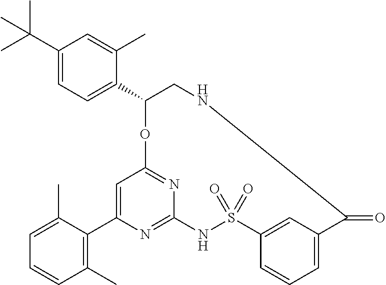 [SMILES: Cc1cc(C(C)(C)C)ccc1[C@@H]1CNC(=O)c2cccc(c2)S(=O)(=O)Nc2nc(cc(-c3c(C)cccc3C)n2)O1] | +++ | +++ | +++ | ++ | | |
| 171 | 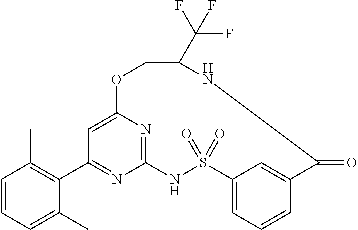 [SMILES: Cc1cccc(C)c1-c1cc2nc(n1)NS(=O)(=O)c1cccc(c1)C(=O)NC(C(F)(F)F)CO2] | | | ND | + | | |
| 120 | 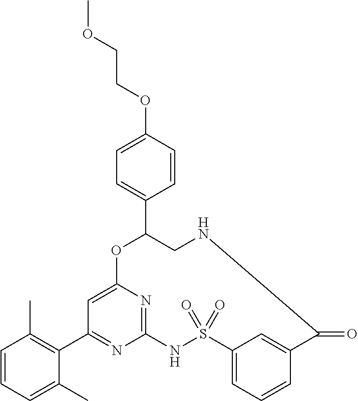 [SMILES: COCCOc1ccc(C2CNC(=O)c3cccc(c3)S(=O)(=O)Nc3nc(cc(-c4c(C)cccc4C)n3)O2)cc1] | | | ND | + | | |
| 119 | 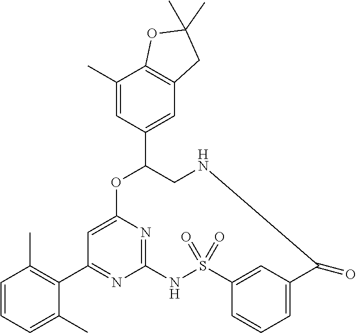 [SMILES: Cc1cc(C2CNC(=O)c3cccc(c3)S(=O)(=O)Nc3nc(cc(-c4c(C)cccc4C)n3)O2)cc2c1OC(C)(C)C2] | | | ND | + | | |
| 92 | 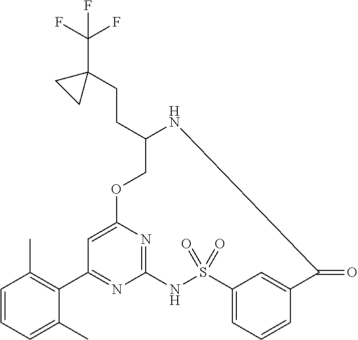 [SMILES: Cc1cccc(C)c1-c1cc2nc(n1)NS(=O)(=O)c1cccc(c1)C(=O)NC(CCC1(C(F)(F)F)CC1)CO2] | | | +++ | +++ | | |
| 230 | 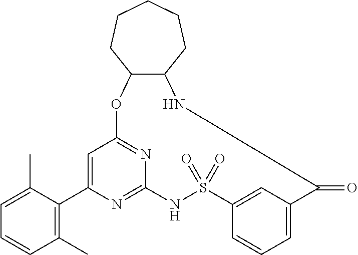 [SMILES: Cc1cccc(C)c1-c1cc2nc(n1)NS(=O)(=O)c1cccc(c1)C(=O)NC1CCCCCC1O2] | | | | | | |
| 289 | 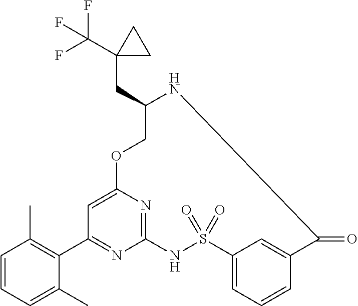 [SMILES: Cc1cccc(C)c1-c1cc2nc(n1)NS(=O)(=O)c1cccc(c1)C(=O)N[C@H](CC1(C(F)(F)F)CC1)CO2] | | | ++ | ++ | | |
| 288 | 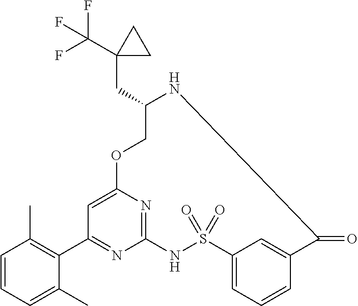 [SMILES: Cc1cccc(C)c1-c1cc2nc(n1)NS(=O)(=O)c1cccc(c1)C(=O)N[C@@H](CC1(C(F)(F)F)CC1)CO2] | | | +++ | +++ | | |
| 275 | 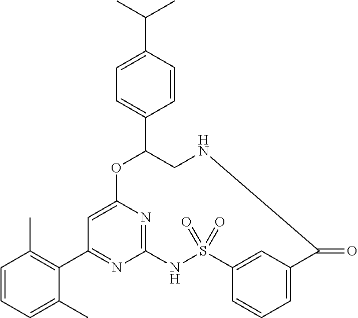 [SMILES: Cc1cccc(C)c1-c1cc2nc(n1)NS(=O)(=O)c1cccc(c1)C(=O)NCC(c1ccc(C(C)C)cc1)O2] | | | +++ | ++ | | |
| 154 | 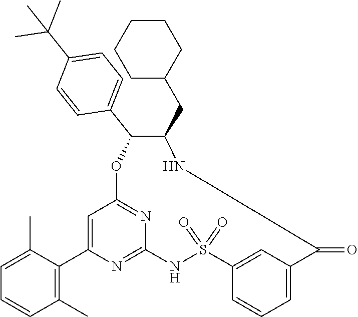 [SMILES: Cc1cccc(C)c1-c1cc2nc(n1)NS(=O)(=O)c1cccc(c1)C(=O)N[C@H](CC1CCCCC1)[C@@H](c1ccc(C(C)(C)C)cc1)O2] | | | +++ | +++ | | |
| 117 | 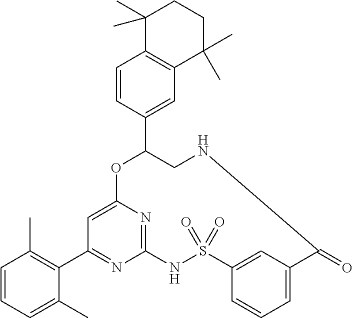 [SMILES: Cc1cccc(C)c1-c1cc2nc(n1)NS(=O)(=O)c1cccc(c1)C(=O)NCC(c1ccc3c(c1)C(C)(C)CCC3(C)C)O2] | | | +++ | +++ | | |
| 116 | 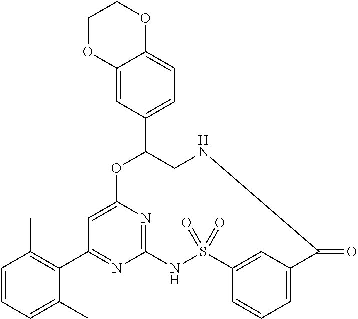 [SMILES: Cc1cccc(C)c1-c1cc2nc(n1)NS(=O)(=O)c1cccc(c1)C(=O)NCC(c1ccc3c(c1)OCCO3)O2] | | | +++ | +++ | | |
| 118 | 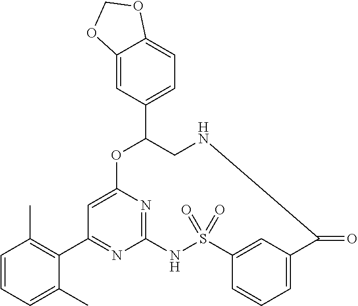 [SMILES: Cc1cccc(C)c1-c1cc2nc(n1)NS(=O)(=O)c1cccc(c1)C(=O)NCC(c1ccc3c(c1)OCO3)O2] | +++ | +++ | +++ | ++ | | |
| 243 | 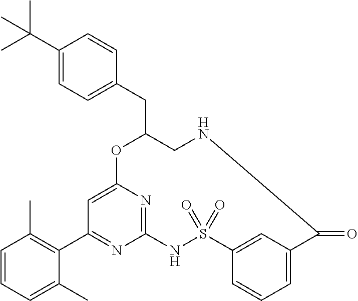 [SMILES: Cc1cccc(C)c1-c1cc2nc(n1)NS(=O)(=O)c1cccc(c1)C(=O)NCC(Cc1ccc(C(C)(C)C)cc1)O2] | | | +++ | +++ | | |
| 9 | 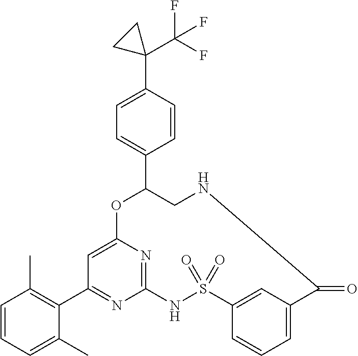 [SMILES: Cc1cccc(C)c1-c1cc2nc(n1)NS(=O)(=O)c1cccc(c1)C(=O)NCC(c1ccc(C3(C(F)(F)F)CC3)cc1)O2] | +++ | +++ | +++ | +++ | | |
| 274 | 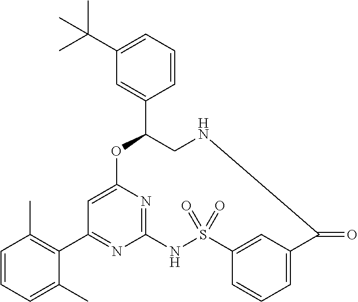 [SMILES: Cc1cccc(C)c1-c1cc2nc(n1)NS(=O)(=O)c1cccc(c1)C(=O)NC[C@H](c1cccc(C(C)(C)C)c1)O2] | +++ | +++ | + | + | | |
| 273 | 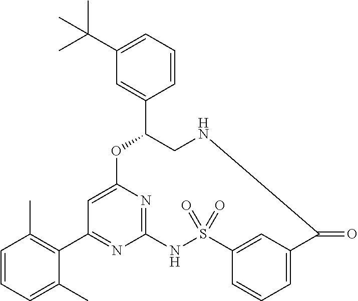 [SMILES: Cc1cccc(C)c1-c1cc2nc(n1)NS(=O)(=O)c1cccc(c1)C(=O)NC[C@@H](c1cccc(C(C)(C)C)c1)O2] | | | +++ | +++ | | |
| 272 | 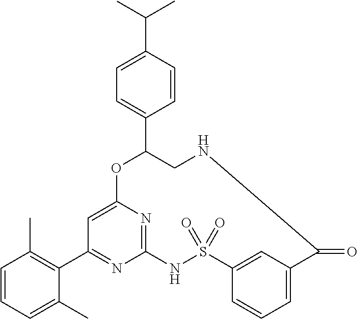 [SMILES: Cc1cccc(C)c1-c1cc2nc(n1)NS(=O)(=O)c1cccc(c1)C(=O)NCC(c1ccc(C(C)C)cc1)O2] | +++ | +++ | ++ | + | | |
| 271 | 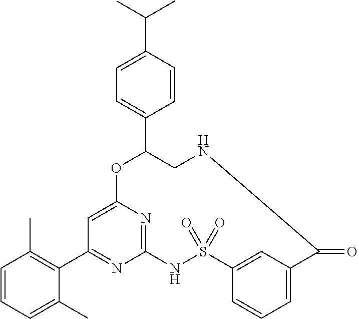 [SMILES: Cc1cccc(C)c1-c1cc2nc(n1)NS(=O)(=O)c1cccc(c1)C(=O)NCC(c1ccc(C(C)C)cc1)O2] | +++ | +++ | +++ | +++ | | |
| 270 | 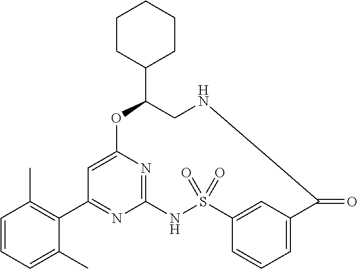 [SMILES: Cc1cccc(C)c1-c1cc2nc(n1)NS(=O)(=O)c1cccc(c1)C(=O)NC[C@H](C1CCCCC1)O2] | +++ | +++ | ND | + | | |
| 269 | 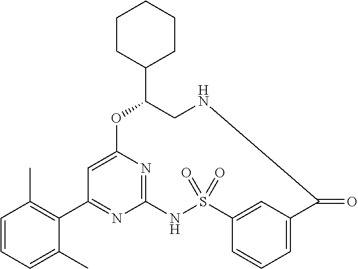 [SMILES: Cc1cccc(C)c1-c1cc2nc(n1)NS(=O)(=O)c1cccc(c1)C(=O)NC[C@@H](C1CCCCC1)O2] | +++ | +++ | | | | |
| 284 | 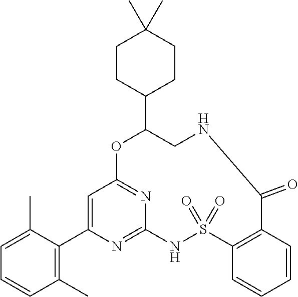 [SMILES: Cc1cccc(C)c1-c1cc2nc(n1)NS(=O)(=O)c1ccccc1C(=O)NCC(C1CCC(C)(C)CC1)O2] | +++ | +++ | +++ | +++ | | |
| 115 | 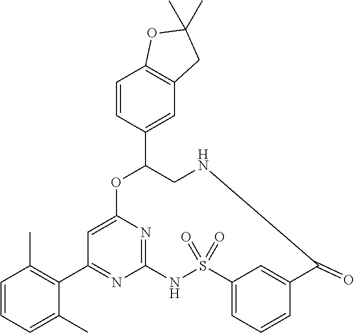 [SMILES: Cc1cccc(C)c1-c1cc2nc(n1)NS(=O)(=O)c1cccc(c1)C(=O)NCC(c1ccc3c(c1)CC(C)(C)O3)O2] | +++ | +++ | ++ | ++ | | |
| 242 | 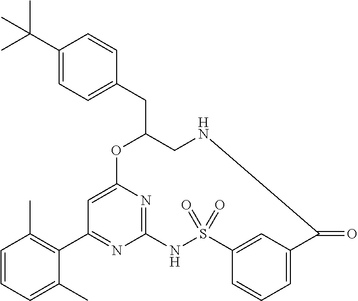 [SMILES: Cc1cccc(C)c1-c1cc2nc(n1)NS(=O)(=O)c1cccc(c1)C(=O)NCC(Cc1ccc(C(C)(C)C)cc1)O2] | | | +++ | ++ | | |
| 241 | 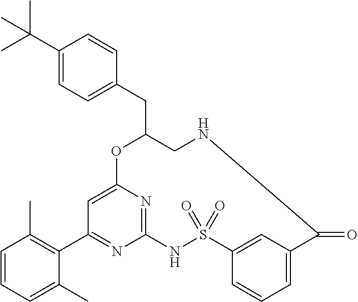 [SMILES: Cc1cccc(C)c1-c1cc2nc(n1)NS(=O)(=O)c1cccc(c1)C(=O)NCC(Cc1ccc(C(C)(C)C)cc1)O2] | | | +++ | +++ | | |
| 267 | 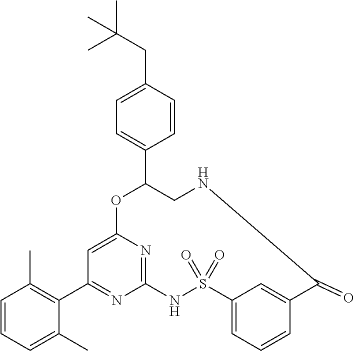 [SMILES: Cc1cccc(C)c1-c1cc2nc(n1)NS(=O)(=O)c1cccc(c1)C(=O)NCC(c1ccc(CC(C)(C)C)cc1)O2] | | | +++ | +++ | | |
| 268 | 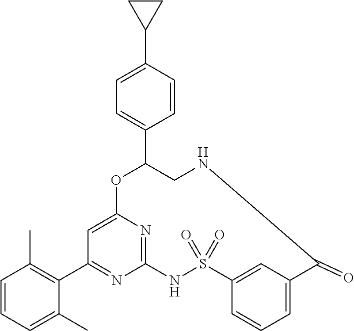 [SMILES: Cc1cccc(C)c1-c1cc2nc(n1)NS(=O)(=O)c1cccc(c1)C(=O)NCC(c1ccc(C3CC3)cc1)O2] | | | +++ | +++ | | |
| 10 | 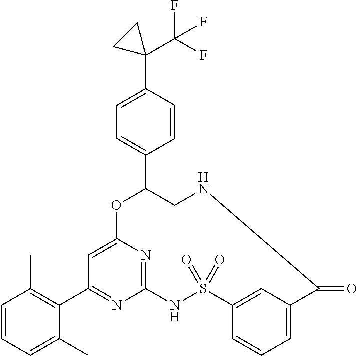 [SMILES: Cc1cccc(C)c1-c1cc2nc(n1)NS(=O)(=O)c1cccc(c1)C(=O)NCC(c1ccc(C3(C(F)(F)F)CC3)cc1)O2] | | | +++ | ++ | | |
| 11 | 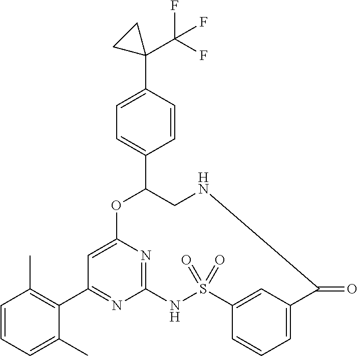 [SMILES: Cc1cccc(C)c1-c1cc2nc(n1)NS(=O)(=O)c1cccc(c1)C(=O)NCC(c1ccc(C3(C(F)(F)F)CC3)cc1)O2] | | | +++ | +++ | +++ | +++ |
| 152 | 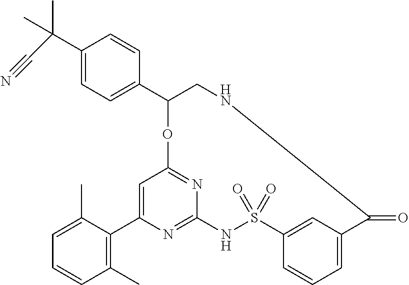 [SMILES: Cc1cccc(C)c1-c1cc2nc(n1)NS(=O)(=O)c1cccc(c1)C(=O)NCC(c1ccc(C(C)(C)C#N)cc1)O2] | | | ND | + | | |
| 153 | 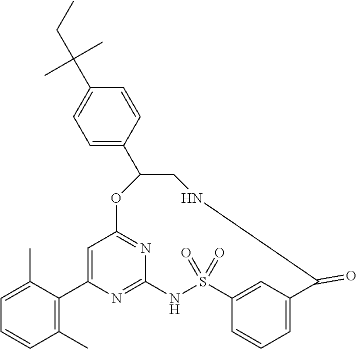 [SMILES: CCC(C)(C)c1ccc(C2CNC(=O)c3cccc(c3)S(=O)(=O)Nc3nc(cc(-c4c(C)cccc4C)n3)O2)cc1] | | | + | + | | |
| 400 | 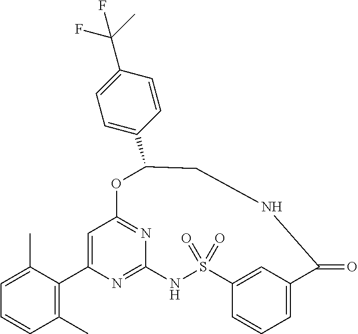 [SMILES: Cc1cccc(C)c1-c1cc2nc(n1)NS(=O)(=O)c1cccc(c1)C(=O)NC[C@@H](c1ccc(C(C)(F)F)cc1)O2] | | | ++ | + | | |
| 399 | 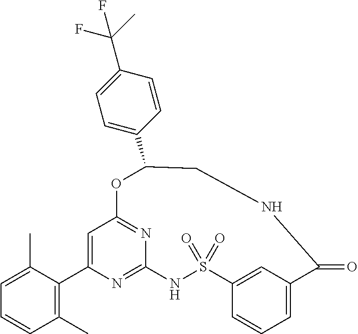 [SMILES: Cc1cccc(C)c1-c1cc2nc(n1)NS(=O)(=O)c1cccc(c1)C(=O)NC[C@@H](c1ccc(C(C)(F)F)cc1)O2] | | | +++ | +++ | | |
| 229 | 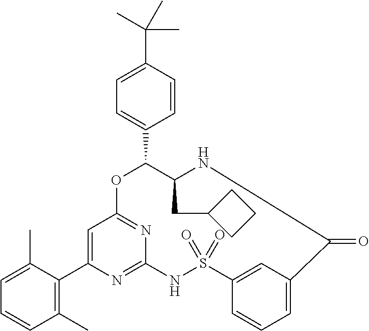 [SMILES: Cc1cccc(C)c1-c1cc2nc(n1)NS(=O)(=O)c1cccc(c1)C(=O)N[C@@H](CC1CCC1)[C@@H](c1ccc(C(C)(C)C)cc1)O2] | | | +++ | +++ | | |

The following table represent CFTR modulating activity for representative compounds of the disclosure generated using one or more of the assays disclosed herein (EC₅₀: +++ is <1 μM; ++ is 1−<3 μM; + is 3−<30 μM; and ND is “not detected in this assay.” % Activity: +++ is >60%; ++ is 30-60%; + is <30%).

TABLE 9
Biological activity data

| | | | 3T3 | Ent. | Ent. A | | Ent. B |
|---|---|---|---|---|---|---|---|
| | | 3T3 | Max | A | Max | Ent. B | Max |
| Compd | | EC₅₀ | Activity | EC₅₀ | Activity | EC₅₀ | Activity |
| Number | Structure | (μM) | (%) | (μM) | (%) | (μM) | (%) |
| 228 | 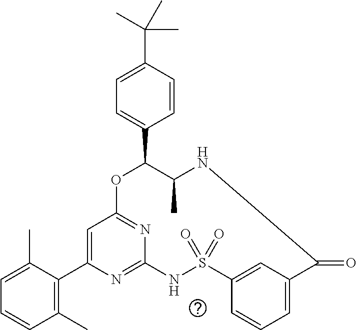 [SMILES: Cc1cccc(C)c1-c1cc2nc(n1)NS(=O)(=O)c1cccc(c1)C(=O)N[C@@H](C)[C@H](c1ccc(C(C)(C)C)cc1)O2] | | | +++ | + | | |
| 133 | 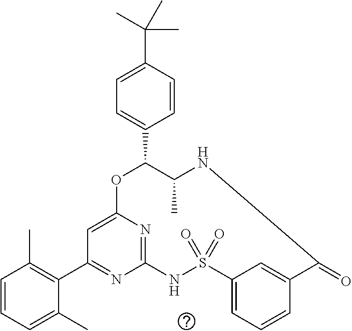 [SMILES: Cc1cccc(C)c1-c1cc2nc(n1)NS(=O)(=O)c1cccc(c1)C(=O)N[C@H](C)[C@@H](c1ccc(C(C)(C)C)cc1)O2] | | | +++ | +++ | | |
| 132 | 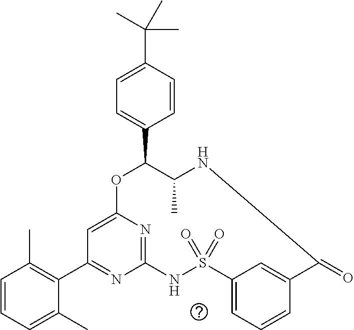 [SMILES: Cc1cccc(C)c1-c1cc2nc(n1)NS(=O)(=O)c1cccc(c1)C(=O)N[C@H](C)[C@H](c1ccc(C(C)(C)C)cc1)O2] | | | +++ | +++ | | |
| 44 | 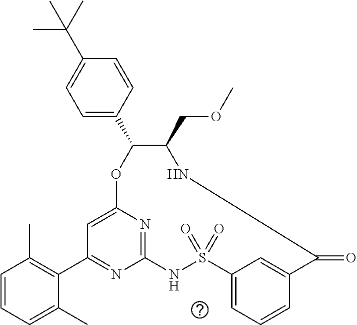 [SMILES: COC[C@H]1NC(=O)c2cccc(c2)S(=O)(=O)Nc2nc(cc(-c3c(C)cccc3C)n2)O[C@@H]1c1ccc(C(C)(C)C)cc1] | | | +++ | +++ | | |
| 45 | 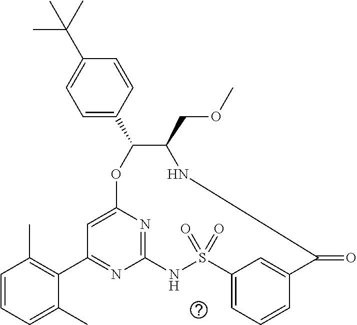 [SMILES: COC[C@H]1NC(=O)c2cccc(c2)S(=O)(=O)Nc2nc(cc(-c3c(C)cccc3C)n2)O[C@@H]1c1ccc(C(C)(C)C)cc1] | | | +++ | +++ | | |
| 265 | 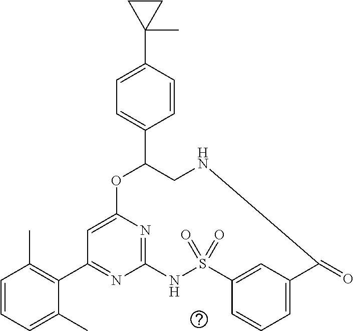 [SMILES: Cc1cccc(C)c1-c1cc2nc(n1)NS(=O)(=O)c1cccc(c1)C(=O)NCC(c1ccc(C3(C)CC3)cc1)O2] | | | +++ | ++ | | |
| 266 | 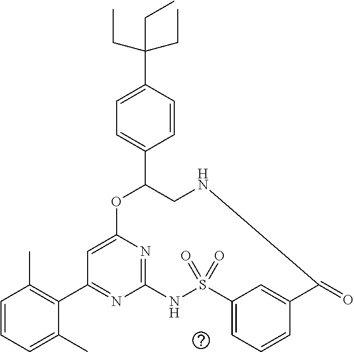 [SMILES: CCC(CC)(CC)c1ccc(C2CNC(=O)c3cccc(c3)S(=O)(=O)Nc3nc(cc(-c4c(C)cccc4C)n3)O2)cc1] | | | +++ | ++ | | |
| 93 | 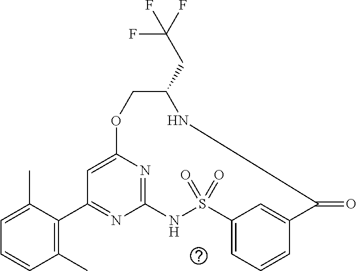 [SMILES: Cc1cccc(C)c1-c1cc2nc(n1)NS(=O)(=O)c1cccc(c1)C(=O)N[C@@H](CC(F)(F)F)CO2] | | | + | ++ | | |
| 94 | 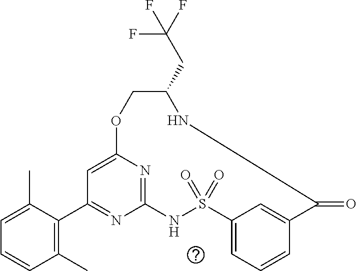 [SMILES: Cc1cccc(C)c1-c1cc2nc(n1)NS(=O)(=O)c1cccc(c1)C(=O)N[C@@H](CC(F)(F)F)CO2] | | | ++ | +++ | | |
| 264 | 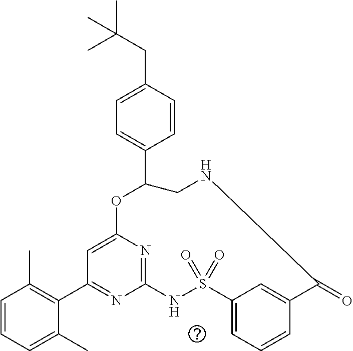 [SMILES: Cc1cccc(C)c1-c1cc2nc(n1)NS(=O)(=O)c1cccc(c1)C(=O)NCC(c1ccc(CC(C)(C)C)cc1)O2] | | | +++ | + | | |
| 263 | 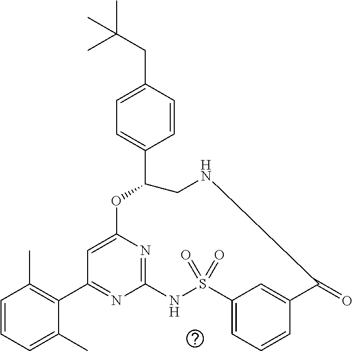 [SMILES: Cc1cccc(C)c1-c1cc2nc(n1)NS(=O)(=O)c1cccc(c1)C(=O)NC[C@@H](c1ccc(CC(C)(C)C)cc1)O2] | | | +++ | +++ | +++ | ++ |
| 262 | 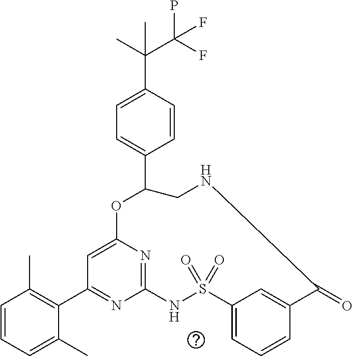 | | | +++ | +++ | | |
| 261 | 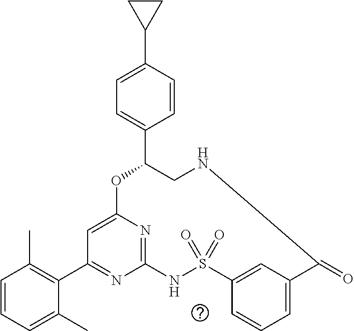 [SMILES: Cc1cccc(C)c1-c1cc2nc(n1)NS(=O)(=O)c1cccc(c1)C(=O)NC[C@@H](c1ccc(C3CC3)cc1)O2] | | | ++ | ++ | | |
| 260 | 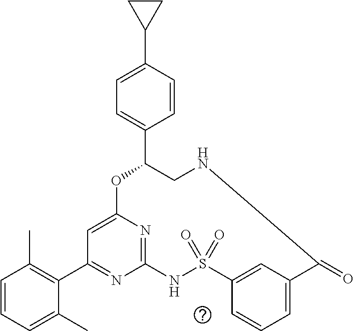 [SMILES: Cc1cccc(C)c1-c1cc2nc(n1)NS(=O)(=O)c1cccc(c1)C(=O)NC[C@@H](c1ccc(C3CC3)cc1)O2] | | | +++ | +++ | | |
| 259 | 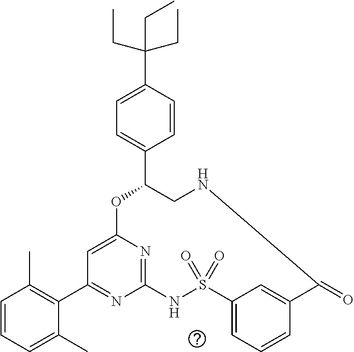 [SMILES: CCC(CC)(CC)c1ccc([C@@H]2CNC(=O)c3cccc(c3)S(=O)(=O)Nc3nc(cc(-c4c(C)cccc4C)n3)O2)cc1] | | | +++ | + | | |
| 258 | 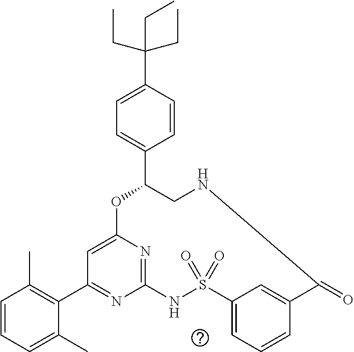 [SMILES: CCC(CC)(CC)c1ccc([C@@H]2CNC(=O)c3cccc(c3)S(=O)(=O)Nc3nc(cc(-c4c(C)cccc4C)n3)O2)cc1] | | | +++ | +++ | | |
| 257 | 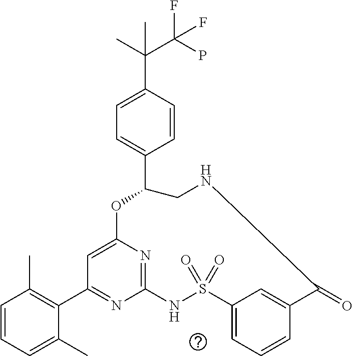 | | | +++ | + | | |
| 256 | 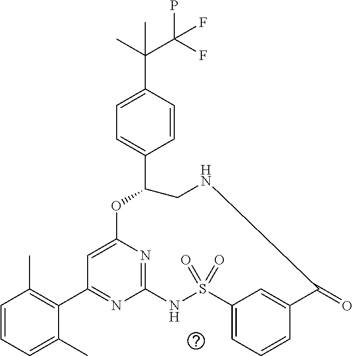 | | | +++ | +++ | | |
| 41 | 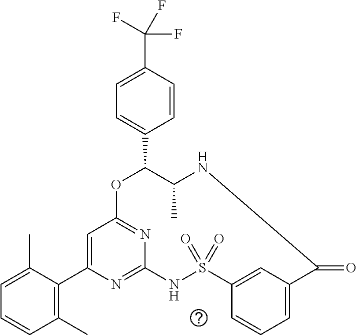 [SMILES: Cc1cccc(C)c1-c1cc2nc(n1)NS(=O)(=O)c1cccc(c1)C(=O)N[C@H](C)[C@@H](c1ccc(C(F)(F)F)cc1)O2] | | | +++ | ++ | | |
| 42 | 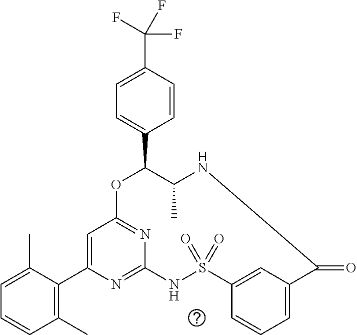 [SMILES: Cc1cccc(C)c1-c1cc2nc(n1)NS(=O)(=O)c1cccc(c1)C(=O)N[C@H](C)[C@H](c1ccc(C(F)(F)F)cc1)O2] | | | +++ | ++ | | |
| 297 | 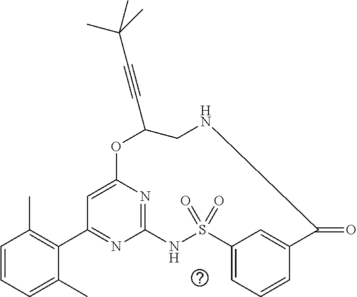 [SMILES: Cc1cccc(C)c1-c1cc2nc(n1)NS(=O)(=O)c1cccc(c1)C(=O)NCC(C#CC(C)(C)C)O2] | | | +++ | ++ | | |
| 227 | 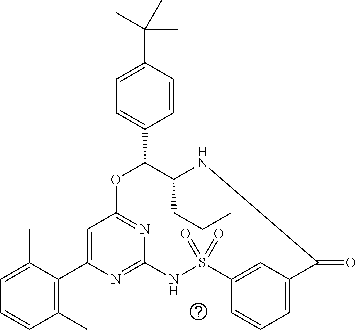 [SMILES: CCC[C@H]1NC(=O)c2cccc(c2)S(=O)(=O)Nc2nc(cc(-c3c(C)cccc3C)n2)O[C@@H]1c1ccc(C(C)(C)C)cc1] | | | +++ | +++ | | |
| 226 | 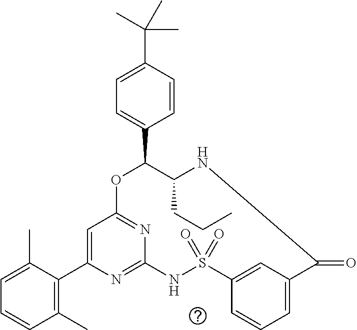 [SMILES: CCC[C@H]1NC(=O)c2cccc(c2)S(=O)(=O)Nc2nc(cc(-c3c(C)cccc3C)n2)O[C@H]1c1ccc(C(C)(C)C)cc1] | | | +++ | +++ | | |
| 131 | 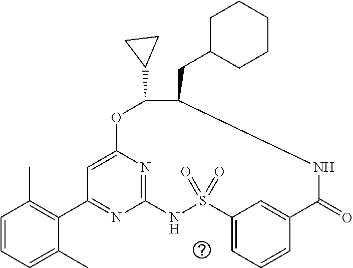 [SMILES: Cc1cccc(C)c1-c1cc2nc(n1)NS(=O)(=O)c1cccc(c1)C(=O)N[C@H](CC1CCCCC1)[C@@H](C1CC1)O2] | | | +++ | +++ | | |
| 240 | 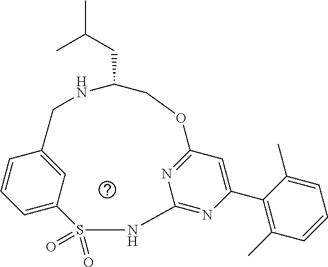 [SMILES: Cc1cccc(C)c1-c1cc2nc(n1)NS(=O)(=O)c1cccc(c1)CN[C@H](CC(C)C)CO2] | | | + | ++ | | |
| 12 | 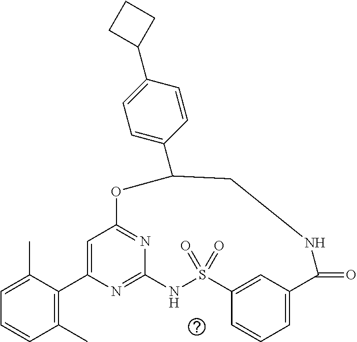 [SMILES: Cc1cccc(C)c1-c1cc2nc(n1)NS(=O)(=O)c1cccc(c1)C(=O)NCC(c1ccc(C3CCC3)cc1)O2] | | | +++ | +++ | | |
| 283 | 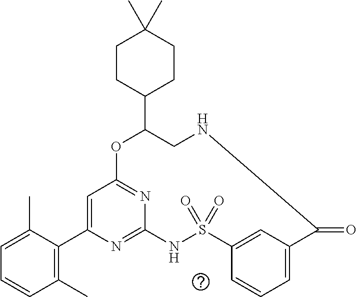 [SMILES: Cc1cccc(C)c1-c1cc2nc(n1)NS(=O)(=O)c1cccc(c1)C(=O)NCC(C1CCC(C)(C)CC1)O2] | | | +++ | +++ | | |
| 13 | 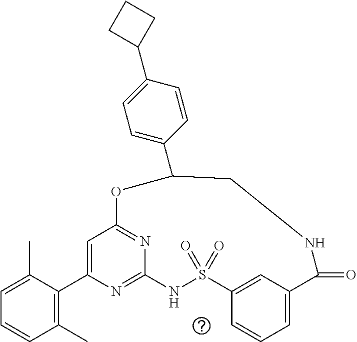 [SMILES: Cc1cccc(C)c1-c1cc2nc(n1)NS(=O)(=O)c1cccc(c1)C(=O)NCC(c1ccc(C3CCC3)cc1)O2] | | | +++ | + | | |
| 14 | 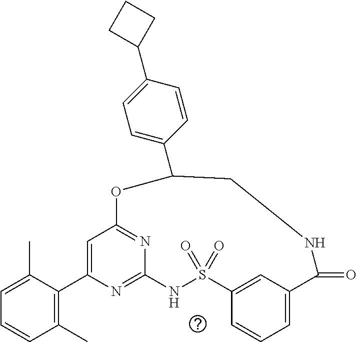 [SMILES: Cc1cccc(C)c1-c1cc2nc(n1)NS(=O)(=O)c1cccc(c1)C(=O)NCC(c1ccc(C3CCC3)cc1)O2] | | | +++ | +++ | | |
| 55 | 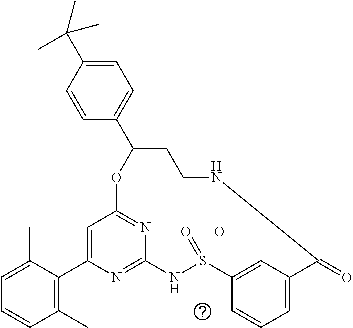 | | | ++ | ++ | | |
| 22 | 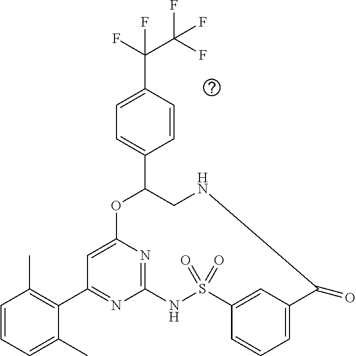 [SMILES: Cc1cccc(C)c1-c1cc2nc(n1)NS(=O)(=O)c1cccc(c1)C(=O)NCC(c1ccc(C(F)(F)C(F)(F)F)cc1)O2] | | | +++ | +++ | | |
| 23 | 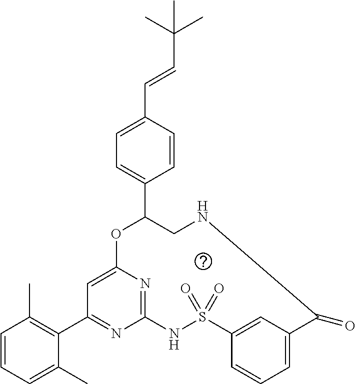 [SMILES: Cc1cccc(C)c1-c1cc2nc(n1)NS(=O)(=O)c1cccc(c1)C(=O)NCC(c1ccc(/C=C/C(C)(C)C)cc1)O2] | | | +++ | +++ | | |
| 24 | 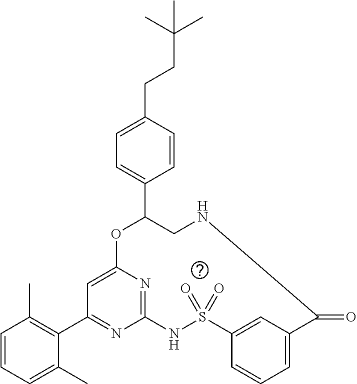 [SMILES: Cc1cccc(C)c1-c1cc2nc(n1)NS(=O)(=O)c1cccc(c1)C(=O)NCC(c1ccc(CCC(C)(C)C)cc1)O2] | | | +++ | +++ | | |
| 89 | 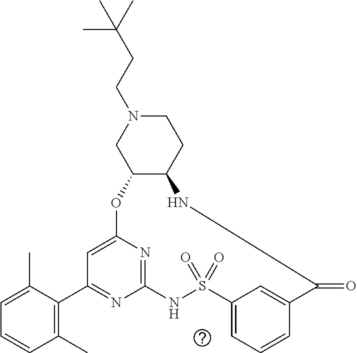 [SMILES: Cc1cccc(C)c1-c1cc2nc(n1)NS(=O)(=O)c1cccc(c1)C(=O)N[C@@H]1CCN(CCC(C)(C)C)C[C@H]1O2] | | | ND | + | | |
| 90 | 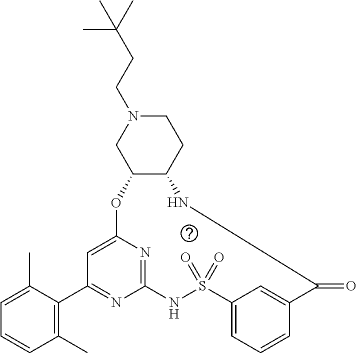 [SMILES: Cc1cccc(C)c1-c1cc2nc(n1)NS(=O)(=O)c1cccc(c1)C(=O)N[C@H]1CCN(CCC(C)(C)C)C[C@H]1O2] | | | ++ | +++ | | |
| 225 | 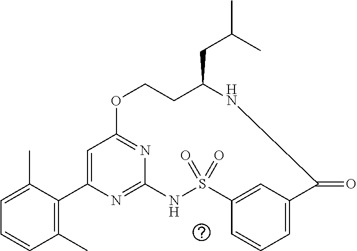 [SMILES: Cc1cccc(C)c1-c1cc2nc(n1)NS(=O)(=O)c1cccc(c1)C(=O)N[C@H](CC(C)C)CCO2] | | | +++ | +++ | | |
| 224 | 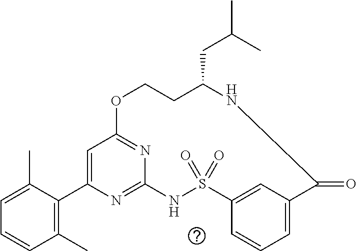 [SMILES: Cc1cccc(C)c1-c1cc2nc(n1)NS(=O)(=O)c1cccc(c1)C(=O)N[C@@H](CC(C)C)CCO2] | | | ++ | ++ | | |
| 43 | 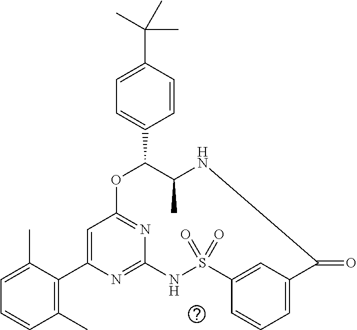 [SMILES: Cc1cccc(C)c1-c1cc2nc(n1)NS(=O)(=O)c1cccc(c1)C(=O)N[C@@H](C)[C@@H](c1ccc(C(C)(C)C)cc1)O2] | | | +++ | +++ | | |
| 185 | 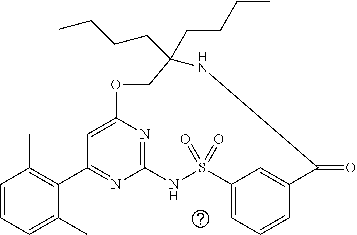 [SMILES: CCCCC1(CCCC)COc2cc(-c3c(C)cccc3C)nc(n2)NS(=O)(=O)c2cccc(c2)C(=O)N1] | | | +++ | +++ | | |
| 184 | 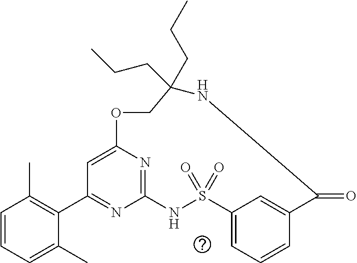 [SMILES: CCCC1(CCC)COc2cc(-c3c(C)cccc3C)nc(n2)NS(=O)(=O)c2cccc(c2)C(=O)N1] | | | +++ | +++ | | |
| 385 | 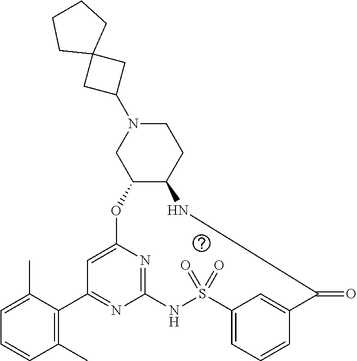 [SMILES: Cc1cccc(C)c1-c1cc2nc(n1)NS(=O)(=O)c1cccc(c1)C(=O)N[C@@H]1CCN(C3CC4(CCCC4)C3)C[C@H]1O2] | | | + | + | | |
| 384 | 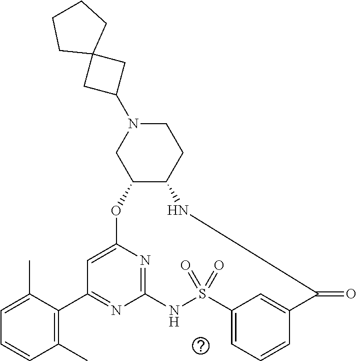 [SMILES: Cc1cccc(C)c1-c1cc2nc(n1)NS(=O)(=O)c1cccc(c1)C(=O)N[C@H]1CCN(C3CC4(CCCC4)C3)C[C@H]1O2] | | | ++ | +++ | | |
| 68 | 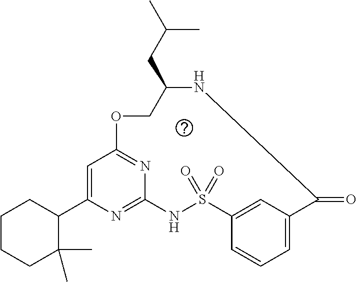 [SMILES: CC(C)C[C@@H]1COc2cc(C3CCCCC3(C)C)nc(n2)NS(=O)(=O)c2cccc(c2)C(=O)N1] | | | ++ | +++ | | |
| 88 | 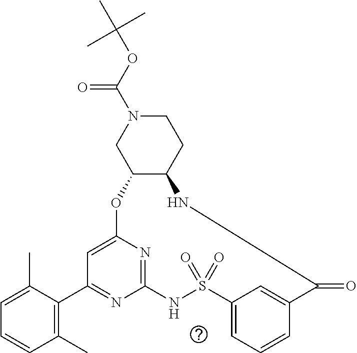 [SMILES: Cc1cccc(C)c1-c1cc2nc(n1)NS(=O)(=O)c1cccc(c1)C(=O)N[C@@H]1CCN(C(=O)OC(C)(C)C)C[C@H]1O2] | | | ND | + | | |
| 87 | 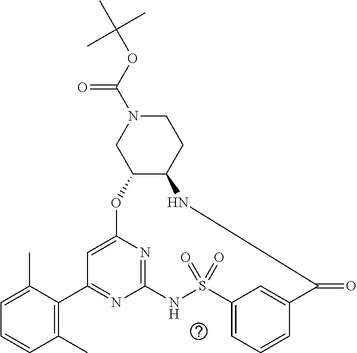 [SMILES: Cc1cccc(C)c1-c1cc2nc(n1)NS(=O)(=O)c1cccc(c1)C(=O)N[C@@H]1CCN(C(=O)OC(C)(C)C)C[C@H]1O2] | | | ++ | +++ | | |
| 223 | 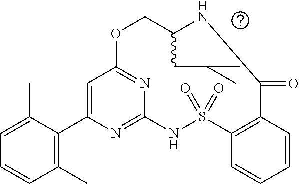 [SMILES: Cc1cccc(C)c1-c1cc2nc(n1)NS(=O)(=O)c1ccccc1C(=O)NC(CC(C)C)CO2] | | | ND | + | | |
| 222 | 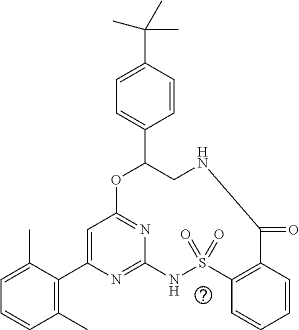 [SMILES: Cc1cccc(C)c1-c1cc2nc(n1)NS(=O)(=O)c1ccccc1C(=O)NCC(c1ccc(C(C)(C)C)cc1)O2] | | | ND | + | | |
| 54 | 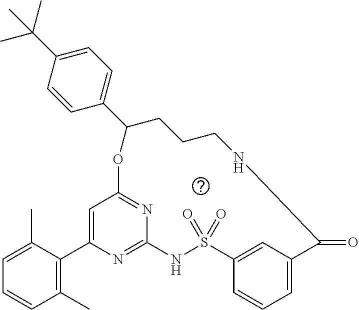 [SMILES: Cc1cccc(C)c1-c1cc2nc(n1)NS(=O)(=O)c1cccc(c1)C(=O)NCCCC(c1ccc(C(C)(C)C)cc1)O2] | | | +++ | +++ | | |
| 398 | 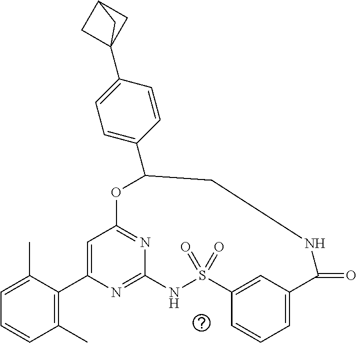 [SMILES: Cc1cccc(C)c1-c1cc2nc(n1)NS(=O)(=O)c1cccc(c1)C(=O)NCC(c1ccc(C34CC(C3)C4)cc1)O2] | | | +++ | +++ | | |
| 397 | 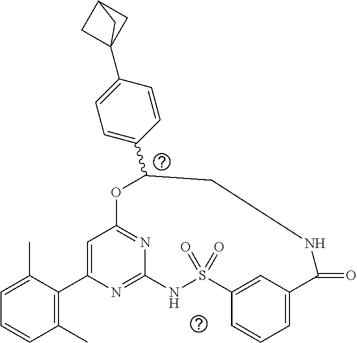 [SMILES: Cc1cccc(C)c1-c1cc2nc(n1)NS(=O)(=O)c1cccc(c1)C(=O)NCC(c1ccc(C34CC(C3)C4)cc1)O2] | | | + | + | | |
| 396 | 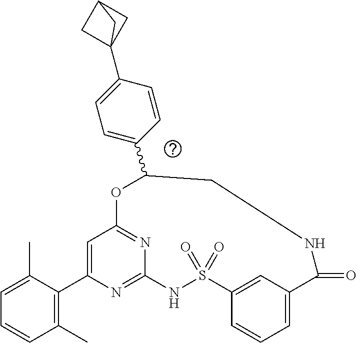 [SMILES: Cc1cccc(C)c1-c1cc2nc(n1)NS(=O)(=O)c1cccc(c1)C(=O)NCC(c1ccc(C34CC(C3)C4)cc1)O2] | | | +++ | +++ | +++ | ++ |
| 15 | 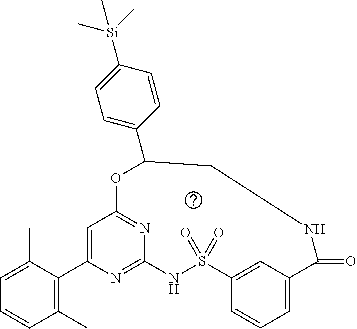 [SMILES: Cc1cccc(C)c1-c1cc2nc(n1)NS(=O)(=O)c1cccc(c1)C(=O)NCC(c1ccc([Si](C)(C)C)cc1)O2] | | | +++ | +++ | | |
| 495 | 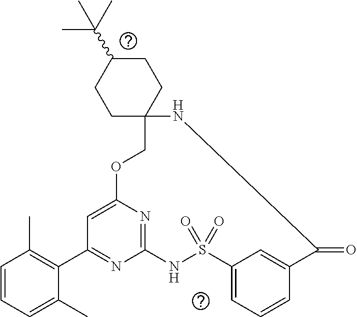 [SMILES: Cc1cccc(C)c1-c1cc2nc(n1)NS(=O)(=O)c1cccc(c1)C(=O)NC1(CCC(C(C)(C)C)CC1)CO2] | | | +++ | +++ | | |
| 110 | 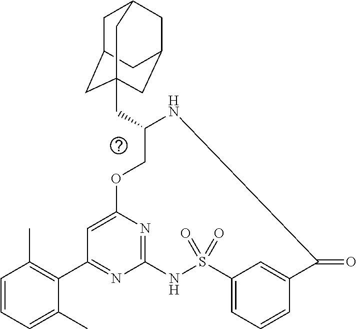 [SMILES: Cc1cccc(C)c1-c1cc2nc(n1)NS(=O)(=O)c1cccc(c1)C(=O)N[C@@H](CC13CC4CC(CC(C4)C1)C3)CO2] | | | +++ | +++ | +++ | +++ |
| 111 | 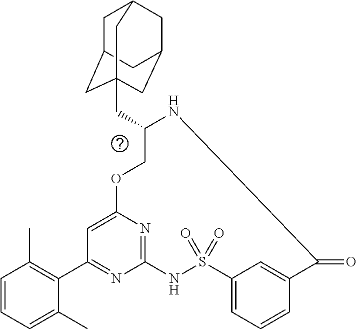 [SMILES: Cc1cccc(C)c1-c1cc2nc(n1)NS(=O)(=O)c1cccc(c1)C(=O)N[C@@H](CC13CC4CC(CC(C4)C1)C3)CO2] | | | +++ | ++ | | |
| 57 | 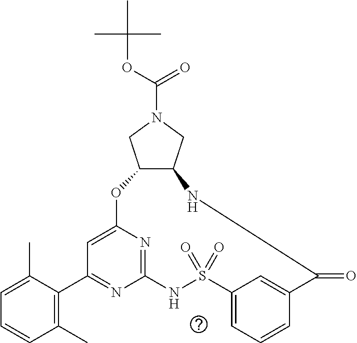 [SMILES: Cc1cccc(C)c1-c1cc2nc(n1)NS(=O)(=O)c1cccc(c1)C(=O)N[C@@H]1CN(C(=O)OC(C)(C)C)C[C@H]1O2] | | | ++ | +++ | ++ | +++ |
| 64 | 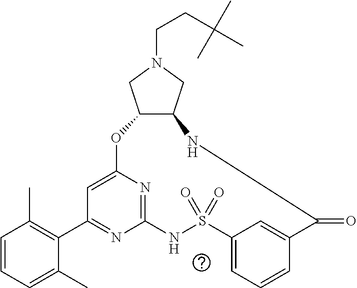 [SMILES: Cc1cccc(C)c1-c1cc2nc(n1)NS(=O)(=O)c1cccc(c1)C(=O)N[C@@H]1CN(CCC(C)(C)C)C[C@H]1O2] | | | +++ | +++ | +++ | ++ |
| 56 | 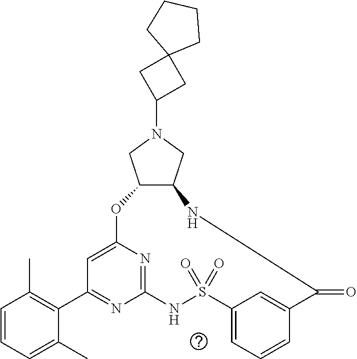 [SMILES: Cc1cccc(C)c1-c1cc2nc(n1)NS(=O)(=O)c1cccc(c1)C(=O)N[C@@H]1CN(C3CC4(CCCC4)C3)C[C@H]1O2] | | | +++ | +++ | +++ | ++ |
| 282 | 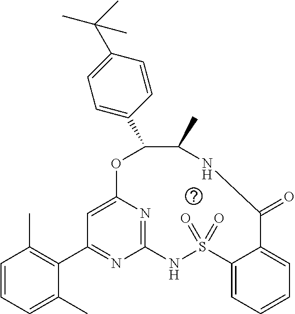 [SMILES: Cc1cccc(C)c1-c1cc2nc(n1)NS(=O)(=O)c1ccccc1C(=O)N[C@H](C)[C@@H](c1ccc(C(C)(C)C)cc1)O2] | | | ND | + | | |
| 16 | 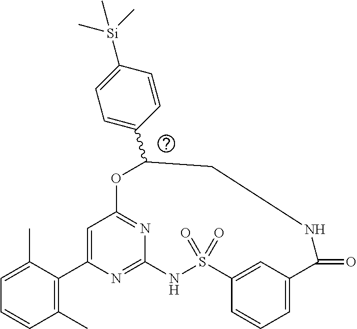 [SMILES: Cc1cccc(C)c1-c1cc2nc(n1)NS(=O)(=O)c1cccc(c1)C(=O)NCC(c1ccc([Si](C)(C)C)cc1)O2] | | | +++ | + | | |
| 17 | 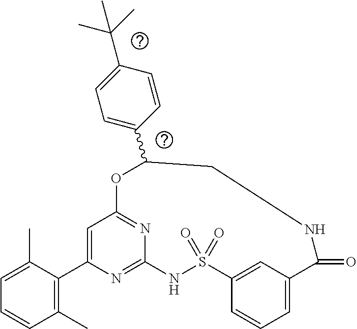 [SMILES: Cc1cccc(C)c1-c1cc2nc(n1)NS(=O)(=O)c1cccc(c1)C(=O)NCC(c1ccc(C(C)(C)C)cc1)O2] | | | +++ | +++ | +++ | +++ |
| 183 | 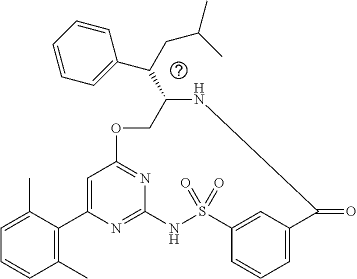 [SMILES: Cc1cccc(C)c1-c1cc2nc(n1)NS(=O)(=O)c1cccc(c1)C(=O)N[C@@H](C(CC(C)C)c1ccccc1)CO2] | | | +++ | +++ | +++ | +++ |
| 182 | 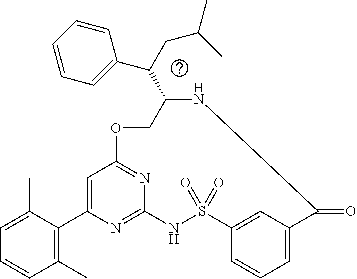 [SMILES: Cc1cccc(C)c1-c1cc2nc(n1)NS(=O)(=O)c1cccc(c1)C(=O)N[C@@H](C(CC(C)C)c1ccccc1)CO2] | | | +++ | ++ | | |
| 181 | 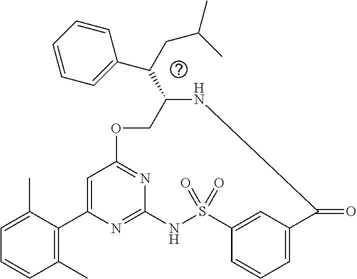 [SMILES: Cc1cccc(C)c1-c1cc2nc(n1)NS(=O)(=O)c1cccc(c1)C(=O)N[C@@H](C(CC(C)C)c1ccccc1)CO2] | | | +++ | +++ | +++ | +++ |
| 180 | 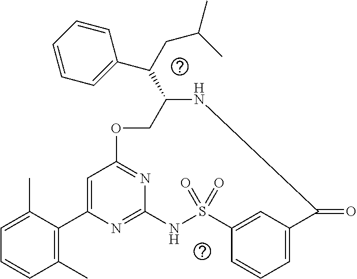 [SMILES: Cc1cccc(C)c1-c1cc2nc(n1)NS(=O)(=O)c1cccc(c1)C(=O)N[C@@H](C(CC(C)C)c1ccccc1)CO2] | | | +++ | ++ | | |
| 383 | 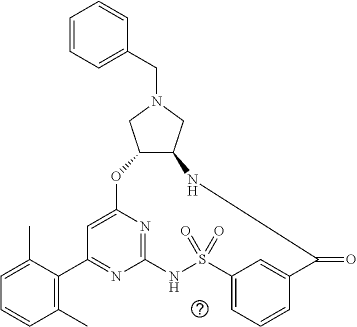 [SMILES: Cc1cccc(C)c1-c1cc2nc(n1)NS(=O)(=O)c1cccc(c1)C(=O)N[C@@H]1CN(Cc3ccccc3)C[C@H]1O2] | | | +++ | +++ | +++ | +++ |
| 382 | 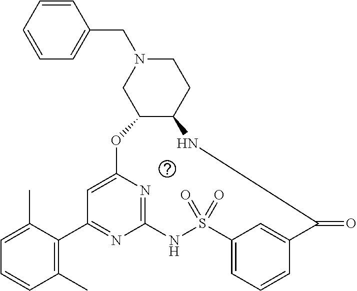 [SMILES: Cc1cccc(C)c1-c1cc2nc(n1)NS(=O)(=O)c1cccc(c1)C(=O)N[C@@H]1CCN(Cc3ccccc3)C[C@H]1O2] | | | +++ | +++ | | |
| 39 | 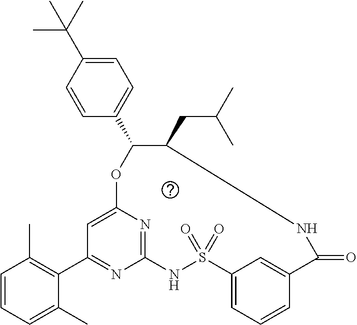 [SMILES: Cc1cccc(C)c1-c1cc2nc(n1)NS(=O)(=O)c1cccc(c1)C(=O)N[C@H](CC(C)C)[C@@H](c1ccc(C(C)(C)C)cc1)O2] | | | +++ | +++ | +++ | +++ |
| 40 | 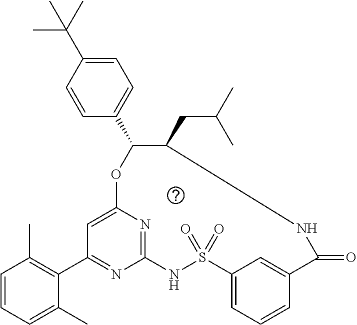 [SMILES: Cc1cccc(C)c1-c1cc2nc(n1)NS(=O)(=O)c1cccc(c1)C(=O)N[C@H](CC(C)C)[C@@H](c1ccc(C(C)(C)C)cc1)O2] | | | +++ | +++ | +++ | +++ |
| 63 | 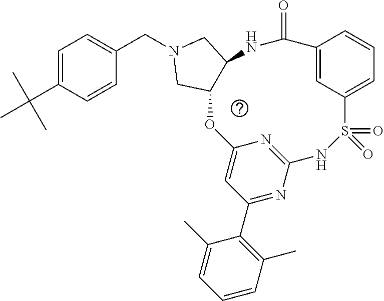 [SMILES: Cc1cccc(C)c1-c1cc2nc(n1)NS(=O)(=O)c1cccc(c1)C(=O)N[C@@H]1CN(Cc3ccc(C(C)(C)C)cc3)C[C@H]1O2] | | | +++ | +++ | +++ | +++ |
| 91 | 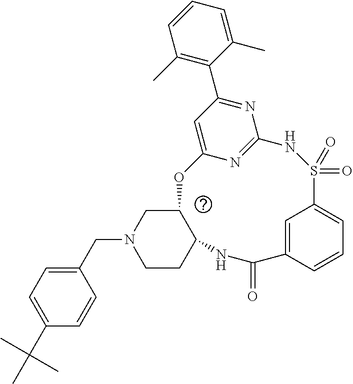 [SMILES: Cc1cccc(C)c1-c1cc2nc(n1)NS(=O)(=O)c1cccc(c1)C(=O)N[C@@H]1CCN(Cc3ccc(C(C)(C)C)cc3)C[C@@H]1O2] | | | +++ | +++ | | |
| 380 | 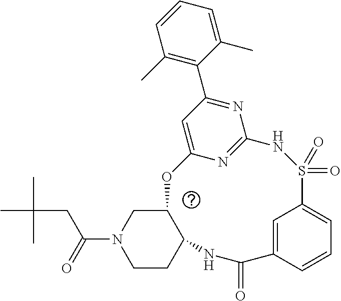 [SMILES: Cc1cccc(C)c1-c1cc2nc(n1)NS(=O)(=O)c1cccc(c1)C(=O)N[C@@H]1CCN(C(=O)CC(C)(C)C)C[C@@H]1O2] | | | + | ++ | | |
| 381 | 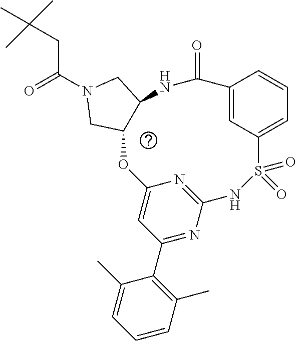 [SMILES: Cc1cccc(C)c1-c1cc2nc(n1)NS(=O)(=O)c1cccc(c1)C(=O)N[C@@H]1CN(C(=O)CC(C)(C)C)C[C@H]1O2] | | | + | ++ | ND | ++ |
| 72 | 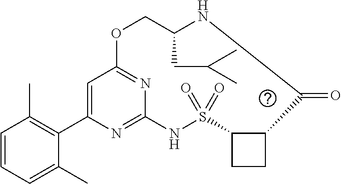 [SMILES: Cc1cccc(C)c1-c1cc2nc(n1)NS(=O)(=O)[C@H]1CC[C@H]1C(=O)N[C@H](CC(C)C)CO2] | | | ND | + | | |
| 71 | 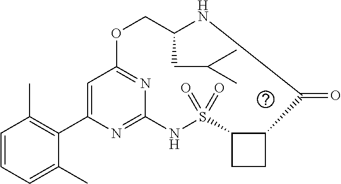 [SMILES: Cc1cccc(C)c1-c1cc2nc(n1)NS(=O)(=O)[C@H]1CC[C@H]1C(=O)N[C@H](CC(C)C)CO2] | | | ND | + | | |
| 151 | 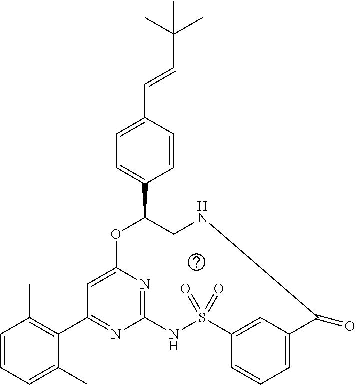 [SMILES: Cc1cccc(C)c1-c1cc2nc(n1)NS(=O)(=O)c1cccc(c1)C(=O)NC[C@H](c1ccc(/C=C/C(C)(C)C)cc1)O2] | | | +++ | +++ | +++ | +++ |
| 150 | 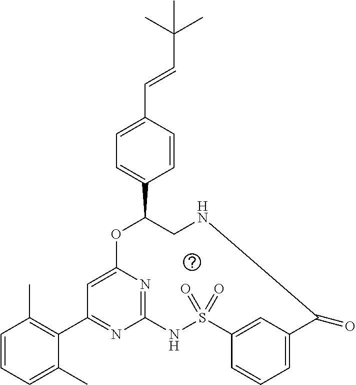 [SMILES: Cc1cccc(C)c1-c1cc2nc(n1)NS(=O)(=O)c1cccc(c1)C(=O)NC[C@H](c1ccc(/C=C/C(C)(C)C)cc1)O2] | | | +++ | ++ | +++ | ++ |
| 379 | 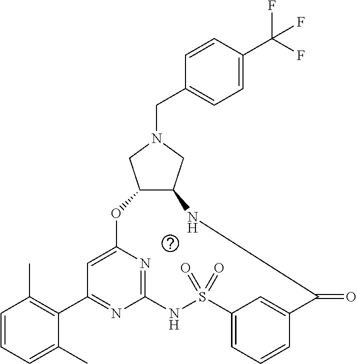 [SMILES: Cc1cccc(C)c1-c1cc2nc(n1)NS(=O)(=O)c1cccc(c1)C(=O)N[C@@H]1CN(Cc3ccc(C(F)(F)F)cc3)C[C@H]1O2] | | | +++ | +++ | +++ | ++ |
| 378 | 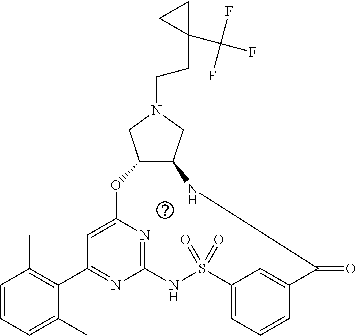 [SMILES: Cc1cccc(C)c1-c1cc2nc(n1)NS(=O)(=O)c1cccc(c1)C(=O)N[C@@H]1CN(CCC3(C(F)(F)F)CC3)C[C@H]1O2] | | | +++ | +++ | +++ | +++ |
| 377 | 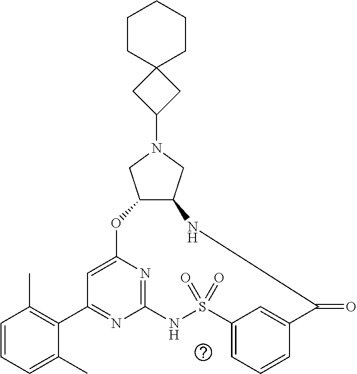 [SMILES: Cc1cccc(C)c1-c1cc2nc(n1)NS(=O)(=O)c1cccc(c1)C(=O)N[C@@H]1CN(C3CC4(CCCCC4)C3)C[C@H]1O2] | | | +++ | +++ | +++ | ++ |
| 106 | 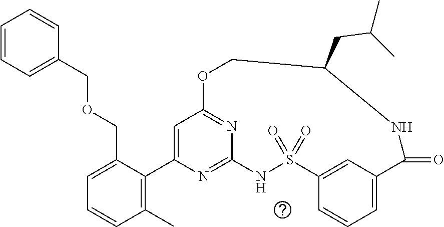 [SMILES: Cc1cccc(COCc2ccccc2)c1-c1cc2nc(n1)NS(=O)(=O)c1cccc(c1)C(=O)N[C@H](CC(C)C)CO2] | | | +++ | +++ | | |
| 149 | 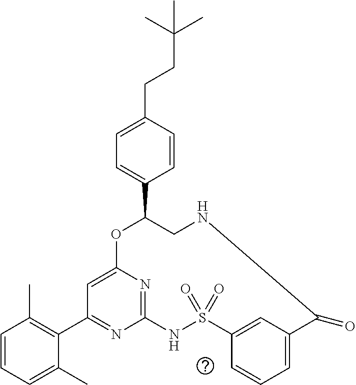 [SMILES: Cc1cccc(C)c1-c1cc2nc(n1)NS(=O)(=O)c1cccc(c1)C(=O)NC[C@H](c1ccc(CCC(C)(C)C)cc1)O2] | | | +++ | +++ | +++ | +++ |
| 148 | 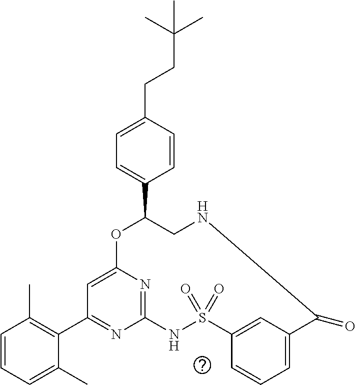 [SMILES: Cc1cccc(C)c1-c1cc2nc(n1)NS(=O)(=O)c1cccc(c1)C(=O)NC[C@H](c1ccc(CCC(C)(C)C)cc1)O2] | | | +++ | ++ | | |
| 147 | 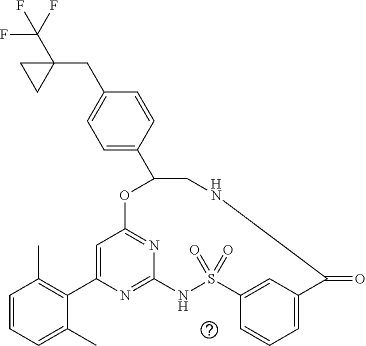 [SMILES: Cc1cccc(C)c1-c1cc2nc(n1)NS(=O)(=O)c1cccc(c1)C(=O)NCC(c1ccc(CC3(C(F)(F)F)CC3)cc1)O2] | | | +++ | +++ | | |
| 130 | 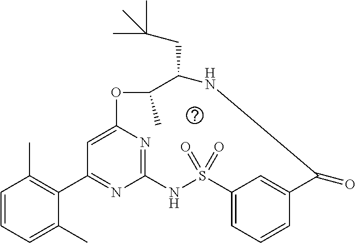 [SMILES: Cc1cccc(C)c1-c1cc2nc(n1)NS(=O)(=O)c1cccc(c1)C(=O)N[C@@H](CC(C)(C)C)[C@H](C)O2] | | | +++ | +++ | | |
| 129 | 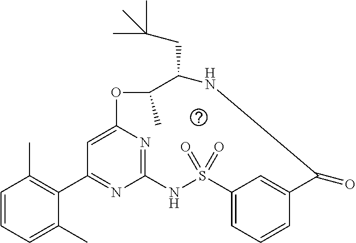 [SMILES: Cc1cccc(C)c1-c1cc2nc(n1)NS(=O)(=O)c1cccc(c1)C(=O)N[C@@H](CC(C)(C)C)[C@H](C)O2] | | | +++ | +++ | | |
| 179 | 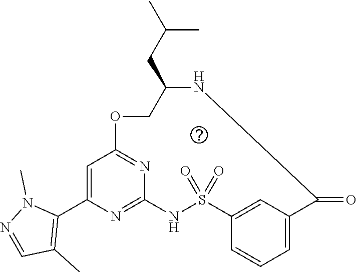 [SMILES: Cc1cnn(C)c1-c1cc2nc(n1)NS(=O)(=O)c1cccc(c1)C(=O)N[C@H](CC(C)C)CO2] | | | + | ++ | | |
| 59 | 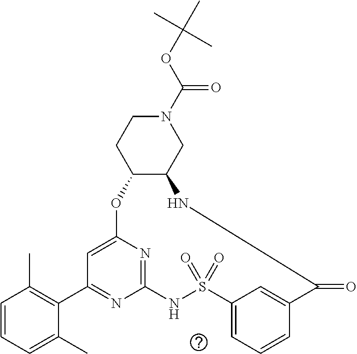 [SMILES: Cc1cccc(C)c1-c1cc2nc(n1)NS(=O)(=O)c1cccc(c1)C(=O)N[C@@H]1CN(C(=O)OC(C)(C)C)CC[C@H]1O2] | | | +++ | +++ | | |
| 376 | 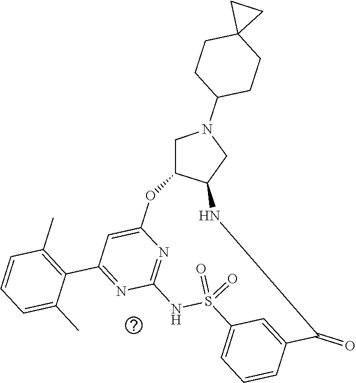 [SMILES: Cc1cccc(C)c1-c1cc2nc(n1)NS(=O)(=O)c1cccc(c1)C(=O)N[C@@H]1CN(C3CCC4(CC3)CC4)C[C@H]1O2] | | | +++ | +++ | +++ | ++ |
| 375 | 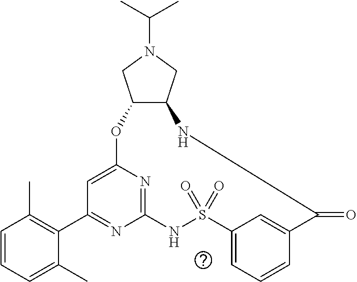 [SMILES: Cc1cccc(C)c1-c1cc2nc(n1)NS(=O)(=O)c1cccc(c1)C(=O)N[C@@H]1CN(C(C)C)C[C@H]1O2] | | | + | +++ | + | ++ |
| 394 | 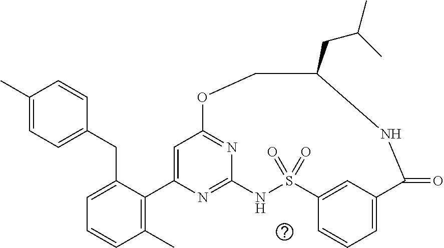 [SMILES: Cc1ccc(Cc2cccc(C)c2-c2cc3nc(n2)NS(=O)(=O)c2cccc(c2)C(=O)N[C@H](CC(C)C)CO3)cc1] | | | +++ | +++ | | |
| 170 | 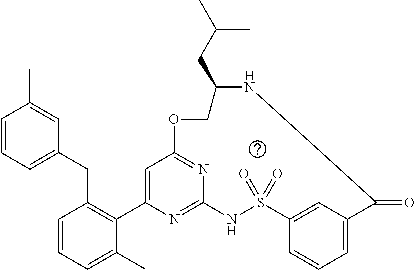 [SMILES: Cc1cccc(Cc2cccc(C)c2-c2cc3nc(n2)NS(=O)(=O)c2cccc(c2)C(=O)N[C@H](CC(C)C)CO3)c1] | | | +++ | +++ | +++ | +++ |
| 101 | 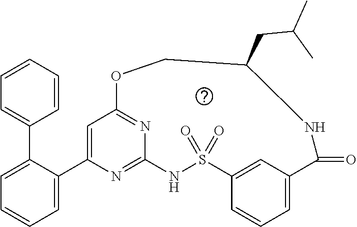 [SMILES: CC(C)C[C@@H]1COc2cc(-c3ccccc3-c3ccccc3)nc(n2)NS(=O)(=O)c2cccc(c2)C(=O)N1] | | | +++ | +++ | | |
| 393 | 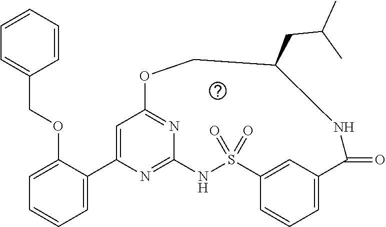 [SMILES: CC(C)C[C@@H]1COc2cc(-c3ccccc3OCc3ccccc3)nc(n2)NS(=O)(=O)c2cccc(c2)C(=O)N1] | | | +++ | +++ | | |
| 108 | 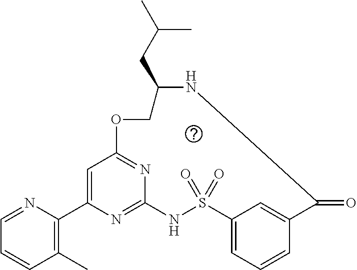 [SMILES: Cc1cccnc1-c1cc2nc(n1)NS(=O)(=O)c1cccc(c1)C(=O)N[C@H](CC(C)C)CO2] | | | + | ++ | | |
| 178 | 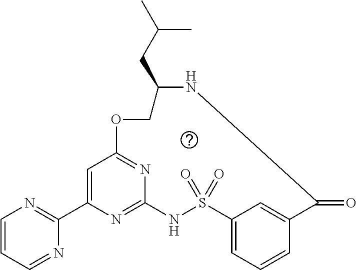 [SMILES: CC(C)C[C@@H]1COc2cc(-c3ncccn3)nc(n2)NS(=O)(=O)c2cccc(c2)C(=O)N1] | | | ND | + | | |
| 496 | 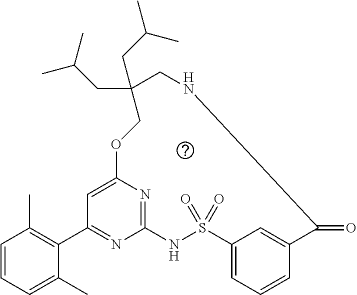 [SMILES: Cc1cccc(C)c1-c1cc2nc(n1)NS(=O)(=O)c1cccc(c1)C(=O)NCC(CC(C)C)(CC(C)C)CO2] | | | +++ | +++ | +++ | +++ |
| 168 | 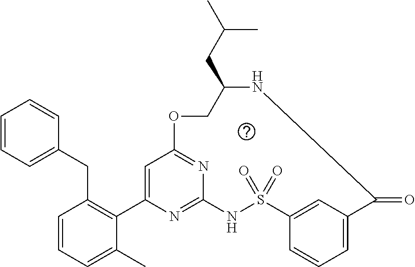 [SMILES: Cc1cccc(Cc2ccccc2)c1-c1cc2nc(n1)NS(=O)(=O)c1cccc(c1)C(=O)N[C@H](CC(C)C)CO2] | | | +++ | +++ | | |
| 61 | 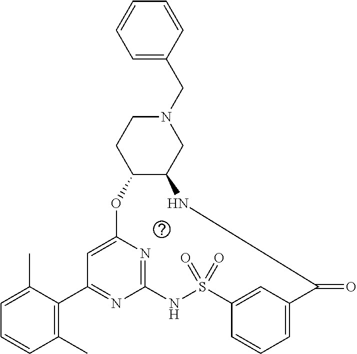 [SMILES: Cc1cccc(C)c1-c1cc2nc(n1)NS(=O)(=O)c1cccc(c1)C(=O)N[C@@H]1CN(Cc3ccccc3)CC[C@H]1O2] | | | +++ | +++ | | |
| 58 | 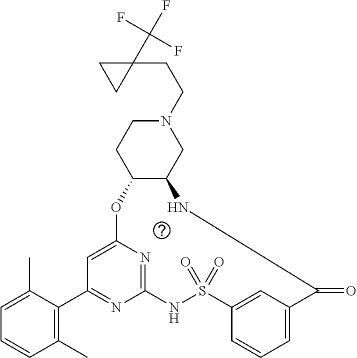 [SMILES: Cc1cccc(C)c1-c1cc2nc(n1)NS(=O)(=O)c1cccc(c1)C(=O)N[C@@H]1CN(CCC3(C(F)(F)F)CC3)CC[C@H]1O2] | | | +++ | +++ | | |
| 374 | 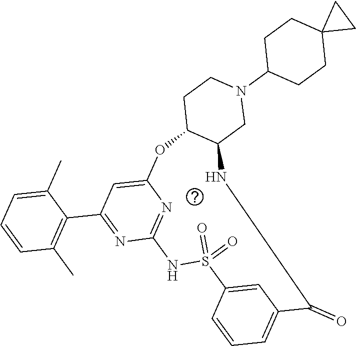 [SMILES: Cc1cccc(C)c1-c1cc2nc(n1)NS(=O)(=O)c1cccc(c1)C(=O)N[C@@H]1CN(C3CCC4(CC3)CC4)CC[C@H]1O2] | | | ++ | +++ | | |
| 60 | 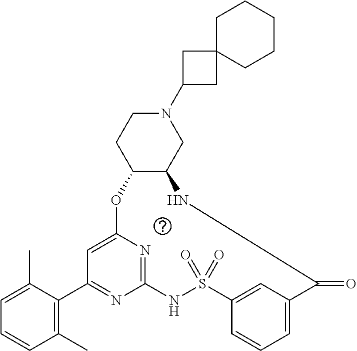 [SMILES: Cc1cccc(C)c1-c1cc2nc(n1)NS(=O)(=O)c1cccc(c1)C(=O)N[C@@H]1CN(C3CC4(CCCCC4)C3)CC[C@H]1O2] | | | +++ | +++ | | |
| 373 | 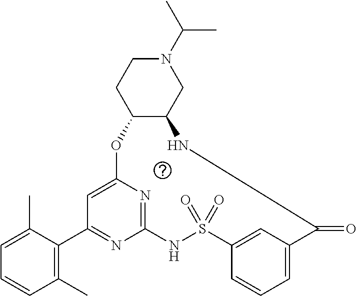 [SMILES: Cc1cccc(C)c1-c1cc2nc(n1)NS(=O)(=O)c1cccc(c1)C(=O)N[C@@H]1CN(C(C)C)CC[C@H]1O2] | | | ++ | +++ | | |
| 78 | 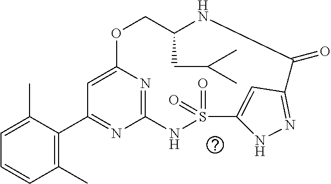 [SMILES: Cc1cccc(C)c1-c1cc2nc(n1)NS(=O)(=O)c1cc(n[nH]1)C(=O)N[C@H](CC(C)C)CO2] | | | + | ++ | + | ++ |
| 395 | 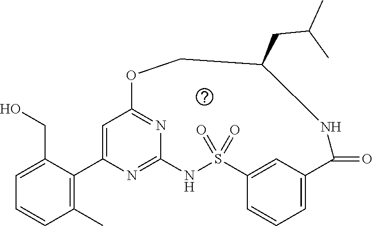 [SMILES: Cc1cccc(CO)c1-c1cc2nc(n1)NS(=O)(=O)c1cccc(c1)C(=O)N[C@H](CC(C)C)CO2] | | | + | ++ | | |
| 372 | 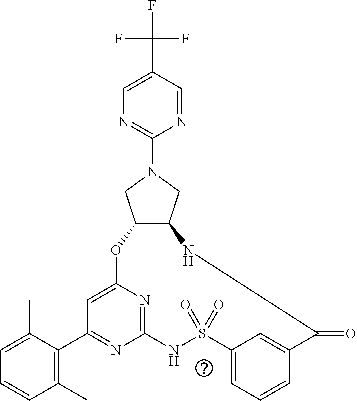 [SMILES: Cc1cccc(C)c1-c1cc2nc(n1)NS(=O)(=O)c1cccc(c1)C(=O)N[C@@H]1CN(c3ncc(C(F)(F)F)cn3)C[C@H]1O2] | | | +++ | +++ | +++ | ++ |
| 62 | 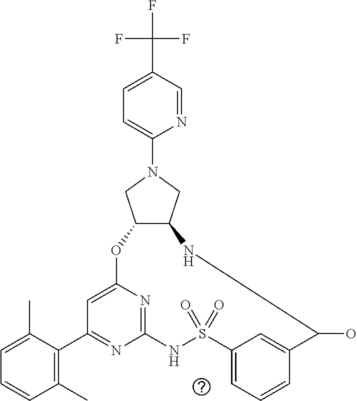 [SMILES: Cc1cccc(C)c1-c1cc2nc(n1)NS(=O)(=O)c1cccc(c1)C(O)N[C@@H]1CN(c3ccc(C(F)(F)F)cn3)C[C@H]1O2] | | | +++ | +++ | +++ | ++ |
| 127 | 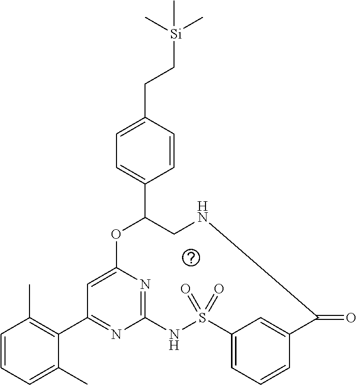 [SMILES: Cc1cccc(C)c1-c1cc2nc(n1)NS(=O)(=O)c1cccc(c1)C(=O)NCC(c1ccc(CC[Si](C)(C)C)cc1)O2] | | | +++ | +++ | | |
| 371 | 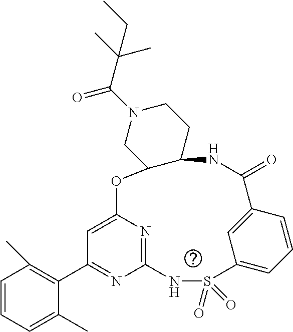 [SMILES: CCC(C)(C)C(=O)N1CC[C@H]2NC(=O)c3cccc(c3)S(=O)(=O)Nc3nc(cc(-c4c(C)cccc4C)n3)OC2C1] | | | + | ++ | | |
| 370 | 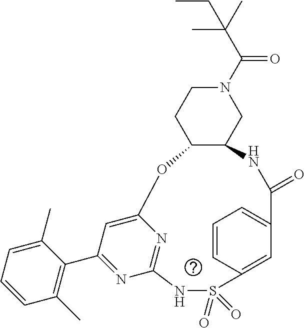 [SMILES: CCC(C)(C)C(=O)N1CC[C@H]2Oc3cc(-c4c(C)cccc4C)nc(n3)NS(=O)(=O)c3cccc(c3)C(=O)N[C@@H]2C1] | | | ++ | +++ | | |
| 369 | 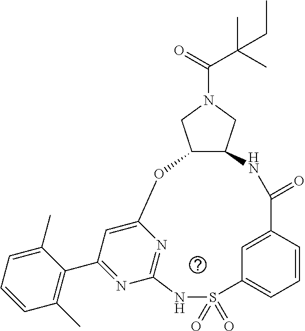 [SMILES: CCC(C)(C)C(=O)N1C[C@H]2NC(=O)c3cccc(c3)S(=O)(=O)Nc3nc(cc(-c4c(C)cccc4C)n3)O[C@@H]2C1] | | | + | +++ | ND | ++ |
| 167 | 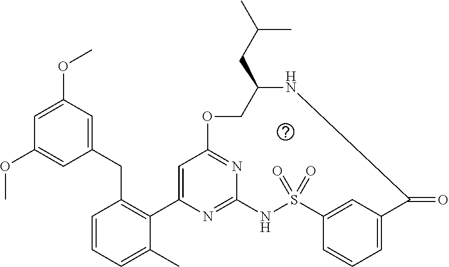 [SMILES: COc1cc(Cc2cccc(C)c2-c2cc3nc(n2)NS(=O)(=O)c2cccc(c2)C(=O)N[C@H](CC(C)C)CO3)cc(OC)c1] | | | +++ | +++ | | |
| 107 | 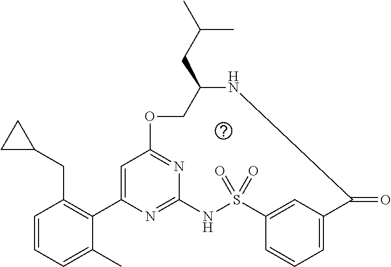 [SMILES: Cc1cccc(CC2CC2)c1-c1cc2nc(n1)NS(=O)(=O)c1cccc(c1)C(=O)N[C@H](CC(C)C)CO2] | | | +++ | +++ | +++ | +++ |
| 392 | 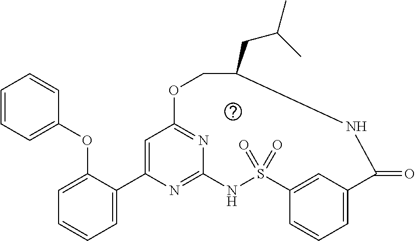 [SMILES: CC(C)C[C@@H]1COc2cc(-c3ccccc3Oc3ccccc3)nc(n2)NS(=O)(=O)c2cccc(c2)C(=O)N1] | | | +++ | ++ | | |
| 128 | 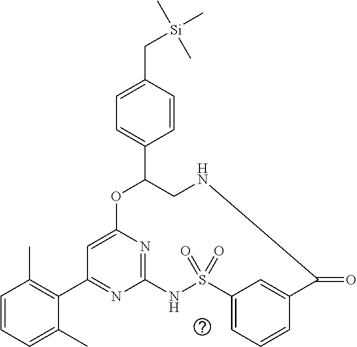 [SMILES: Cc1cccc(C)c1-c1cc2nc(n1)NS(=O)(=O)c1cccc(c1)C(=O)NCC(c1ccc(C[Si](C)(C)C)cc1)O2] | | | +++ | +++ | | |
| 146 | 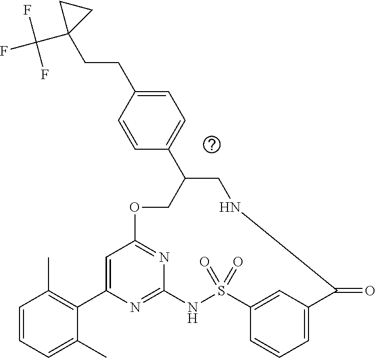 [SMILES: Cc1cccc(C)c1-c1cc2nc(n1)NS(=O)(=O)c1cccc(c1)C(=O)NCC(c1ccc(CCC3(C(F)(F)F)CC3)cc1)CO2] | | | +++ | +++ | +++ | +++ |
| 145 | 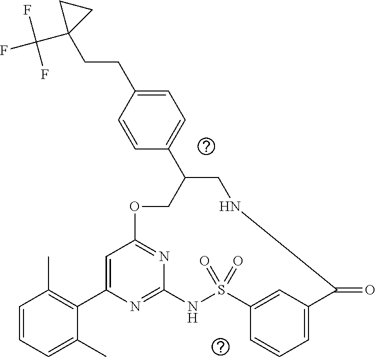 [SMILES: Cc1cccc(C)c1-c1cc2nc(n1)NS(=O)(=O)c1cccc(c1)C(=O)NCC(c1ccc(CCC3(C(F)(F)F)CC3)cc1)CO2] | | | +++ | ++ | | |
| 86 | 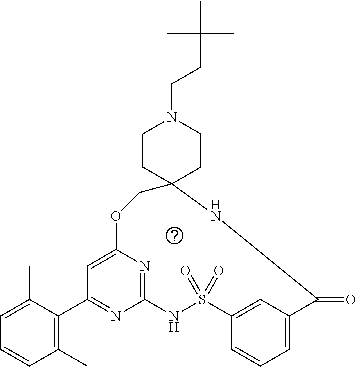 [SMILES: Cc1cccc(C)c1-c1cc2nc(n1)NS(=O)(=O)c1cccc(c1)C(=O)NC1(CCN(CCC(C)(C)C)CC1)CO2] | | | ++ | +++ | | |
| 175 | 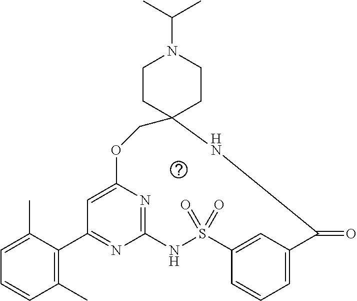 [SMILES: Cc1cccc(C)c1-c1cc2nc(n1)NS(=O)(=O)c1cccc(c1)C(=O)NC1(CCN(C(C)C)CC1)CO2] | | | + | + | | |
| 174 | 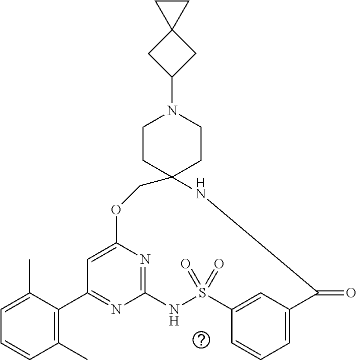 [SMILES: Cc1cccc(C)c1-c1cc2nc(n1)NS(=O)(=O)c1cccc(c1)C(=O)NC1(CCN(C3CC4(CC4)C3)CC1)CO2] | | | ++ | +++ | | |
| 177 | 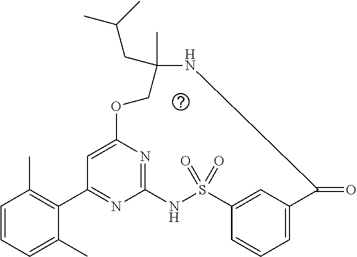 [SMILES: Cc1cccc(C)c1-c1cc2nc(n1)NS(=O)(=O)c1cccc(c1)C(=O)NC(C)(CC(C)C)CO2] | | | +++ | +++ | +++ | +++ |
| 176 | 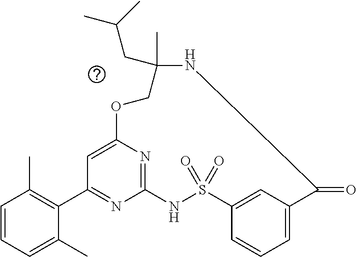 [SMILES: Cc1cccc(C)c1-c1cc2nc(n1)NS(=O)(=O)c1cccc(c1)C(=O)NC(C)(CC(C)C)CO2] | | | +++ | +++ | | |
| 166 | 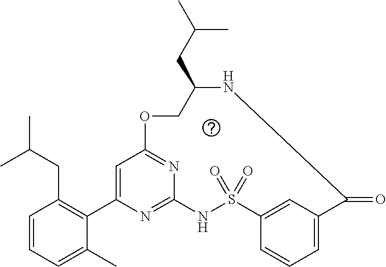 [SMILES: Cc1cccc(CC(C)C)c1-c1cc2nc(n1)NS(=O)(=O)c1cccc(c1)C(=O)N[C@H](CC(C)C)CO2] | | | | | | |
| 165 | 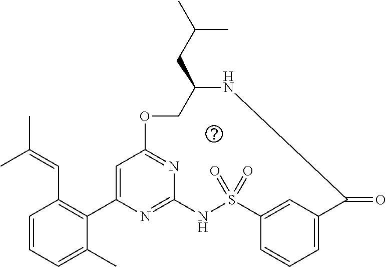 [SMILES: CC(C)=Cc1cccc(C)c1-c1cc2nc(n1)NS(=O)(=O)c1cccc(c1)C(=O)N[C@H](CC(C)C)CO2] | | | | | | |
| 391 | 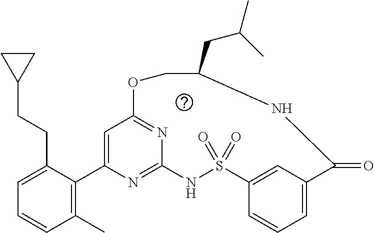 [SMILES: Cc1cccc(CCC2CC2)c1-c1cc2nc(n1)NS(=O)(=O)c1cccc(c1)C(=O)N[C@H](CC(C)C)CO2] | | | | | +++ | +++ |
| 53 | 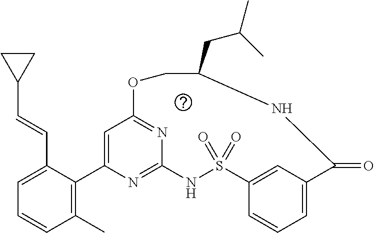 [SMILES: Cc1cccc(/C=C/C2CC2)c1-c1cc2nc(n1)NS(=O)(=O)c1cccc(c1)C(=O)N[C@H](CC(C)C)CO2] | | | | | +++ | +++ |
| 126 | 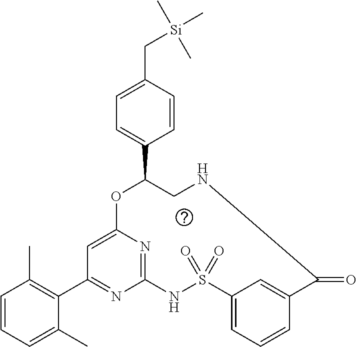 [SMILES: Cc1cccc(C)c1-c1cc2nc(n1)NS(=O)(=O)c1cccc(c1)C(=O)NC[C@H](c1ccc(C[Si](C)(C)C)cc1)O2] | | | | | +++ | ++ |
| 125 | 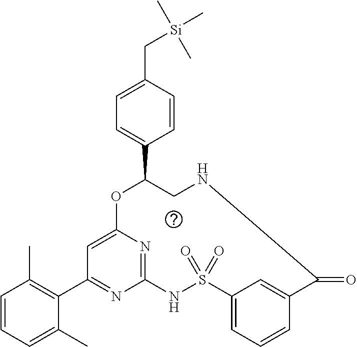 [SMILES: Cc1cccc(C)c1-c1cc2nc(n1)NS(=O)(=O)c1cccc(c1)C(=O)NC[C@H](c1ccc(C[Si](C)(C)C)cc1)O2] | | | | | +++ | +++ |
| 124 | 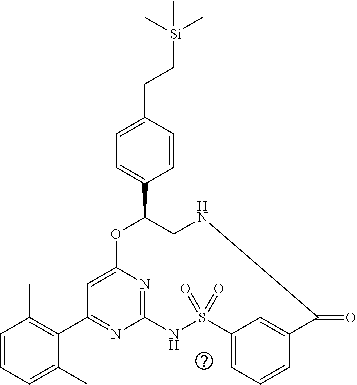 [SMILES: Cc1cccc(C)c1-c1cc2nc(n1)NS(=O)(=O)c1cccc(c1)C(=O)NC[C@H](c1ccc(CC[Si](C)(C)C)cc1)O2] | | | | | +++ | +++ |
| 123 | 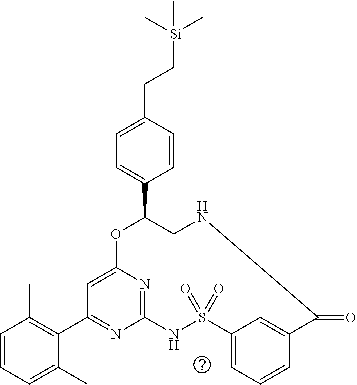 [SMILES: Cc1cccc(C)c1-c1cc2nc(n1)NS(=O)(=O)c1cccc(c1)C(=O)NC[C@H](c1ccc(CC[Si](C)(C)C)cc1)O2] | | | | | +++ | + |
| 221 | 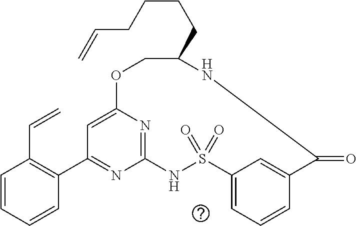 [SMILES: C=CCCCC[C@@H]1COc2cc(-c3ccccc3C=C)nc(n2)NS(=O)(=O)c2cccc(c2)C(=O)N1] | | | | | +++ | +++ |
| 220 | 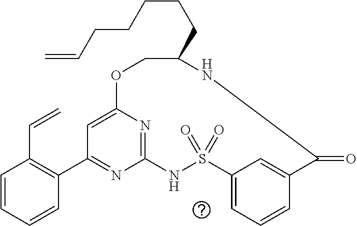 [SMILES: C=CCCCCC[C@@H]1COc2cc(-c3ccccc3C=C)nc(n2)NS(=O)(=O)c2cccc(c2)C(=O)N1] | | | | | +++ | +++ |
| 35 | 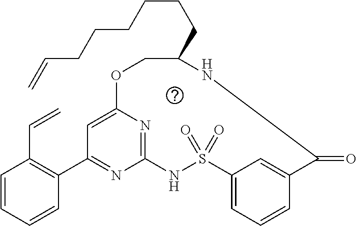 [SMILES: C=CCCCCCC[C@@H]1COc2cc(-c3ccccc3C=C)nc(n2)NS(=O)(=O)c2cccc(c2)C(=O)N1] | | | | | +++ | ++ |
| 389 | 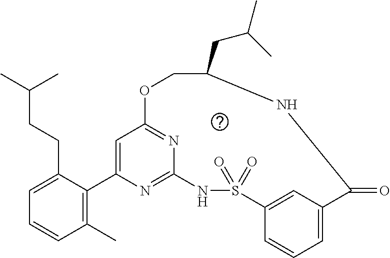 [SMILES: Cc1cccc(CCC(C)C)c1-c1cc2nc(n1)NS(=O)(=O)c1cccc(c1)C(=O)N[C@H](CC(C)C)CO2] | | | | | +++ | +++ |
| 390 | 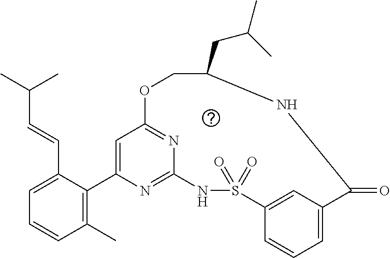 [SMILES: Cc1cccc(/C=C/C(C)C)c1-c1cc2nc(n1)NS(=O)(=O)c1cccc(c1)C(=O)N[C@H](CC(C)C)CO2] | | | | | +++ | +++ |
| 218 | 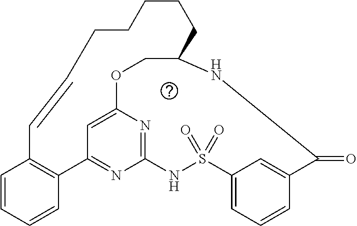 [SMILES: O=C1N[C@@H]2CCCCC/C=C/c3ccccc3-c3cc(nc(n3)NS(=O)(=O)c3cccc1c3)OC2] | | | | | +++ | +++ |
| 36 | 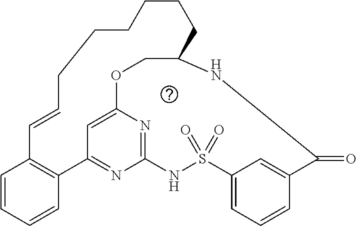 [SMILES: O=C1N[C@@H]2CCCCCC/C=C/c3ccccc3-c3cc(nc(n3)NS(=O)(=O)c3cccc1c3)OC2] | | | | | +++ | +++ |
| 217 | 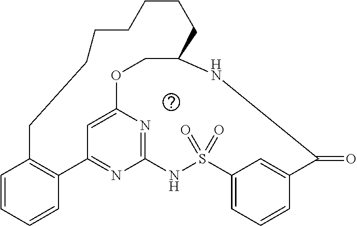 [SMILES: O=C1N[C@@H]2CCCCCCCc3ccccc3-c3cc(nc(n3)NS(=O)(=O)c3cccc1c3)OC2] | | | | | +++ | +++ |
| 37 | 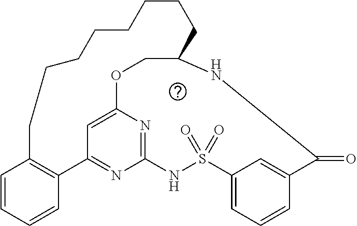 [SMILES: O=C1N[C@@H]2CCCCCCCCc3ccccc3-c3cc(nc(n3)NS(=O)(=O)c3cccc1c3)OC2] | | | | | +++ | +++ |
| 219 | 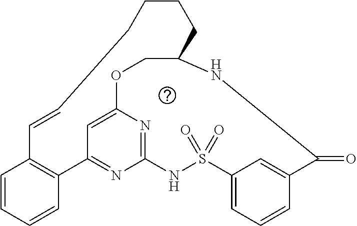 [SMILES: O=C1N[C@@H]2CCCC/C=C/c3ccccc3-c3cc(nc(n3)NS(=O)(=O)c3cccc1c3)OC2] | | | | | +++ | +++ |
| 216 | 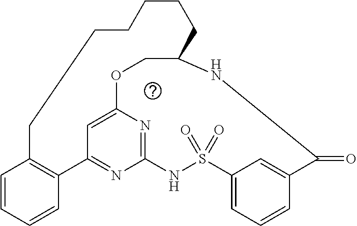 [SMILES: O=C1N[C@@H]2CCCCCCc3ccccc3-c3cc(nc(n3)NS(=O)(=O)c3cccc1c3)OC2] | | | | | +++ | +++ |
| 164 | 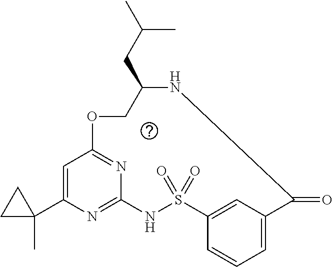 [SMILES: CC(C)C[C@@H]1COc2cc(C3(C)CC3)nc(n2)NS(=O)(=O)c2cccc(c2)C(=O)N1] | | | | | ND | + |
| 163 | 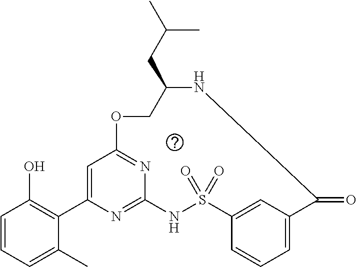 [SMILES: Cc1cccc(O)c1-c1cc2nc(n1)NS(=O)(=O)c1cccc(c1)C(=O)N[C@H](CC(C)C)CO2] | | | | | + | ++ |
| 388 | 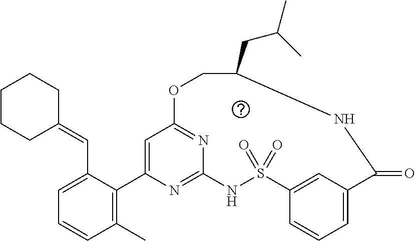 [SMILES: Cc1cccc(C=C2CCCCC2)c1-c1cc2nc(n1)NS(=O)(=O)c1cccc(c1)C(=O)N[C@H](CC(C)C)CO2] | | | | | +++ | ++ |
| 387 | 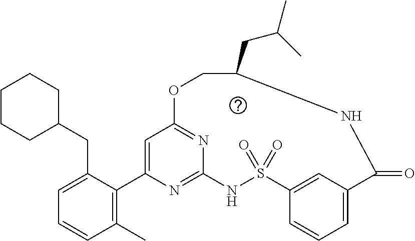 [SMILES: Cc1cccc(CC2CCCCC2)c1-c1cc2nc(n1)NS(=O)(=O)c1cccc(c1)C(=O)N[C@H](CC(C)C)CO2] | | | | | +++ | +++ |
| 70 | 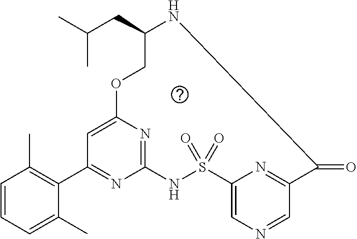 [SMILES: Cc1cccc(C)c1-c1cc2nc(n1)NS(=O)(=O)c1cncc(n1)C(=O)N[C@H](CC(C)C)CO2] | | | | | +++ | +++ |
| 76 | 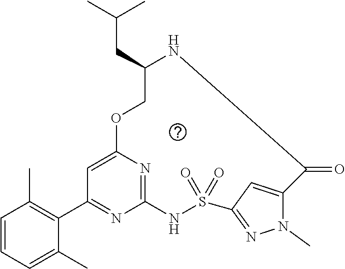 [SMILES: Cc1cccc(C)c1-c1cc2nc(n1)NS(=O)(=O)c1cc(n(C)n1)C(=O)N[C@H](CC(C)C)CO2] | | | | | ++ | +++ |
| 173 | 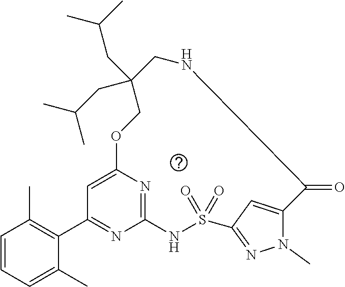 [SMILES: Cc1cccc(C)c1-c1cc2nc(n1)NS(=O)(=O)c1cc(n(C)n1)C(=O)NCC(CC(C)C)(CC(C)C)CO2] | | | | | ++ | ++ |
| 368 | 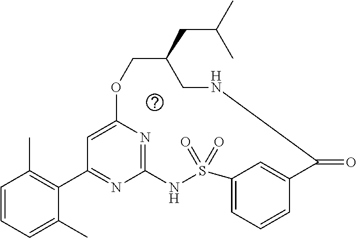 [SMILES: Cc1cccc(C)c1-c1cc2nc(n1)NS(=O)(=O)c1cccc(c1)C(=O)NC[C@H](CC(C)C)CO2] | | | | | +++ | ++ |
| 367 | 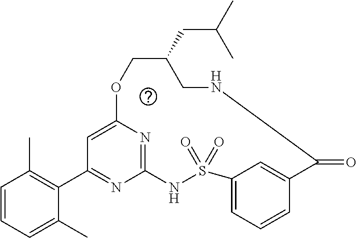 [SMILES: Cc1cccc(C)c1-c1cc2nc(n1)NS(=O)(=O)c1cccc(c1)C(=O)NC[C@@H](CC(C)C)CO2] | | | | | +++ | +++ |
| 172 | 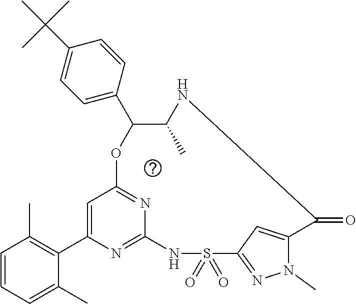 [SMILES: Cc1cccc(C)c1-c1cc2nc(n1)NS(=O)(=O)c1cc(n(C)n1)C(=O)N[C@H](C)C(c1ccc(C(C)(C)C)cc1)O2] | | | | | +++ | ++ |
| 215 | 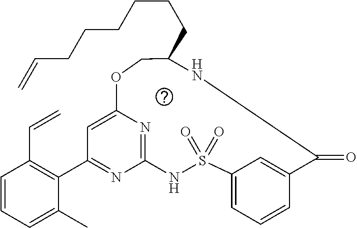 [SMILES: C=CCCCCCC[C@@H]1COc2cc(-c3c(C)cccc3C=C)nc(n2)NS(=O)(=O)c2cccc(c2)C(=O)N1] | | | | | +++ | ++ |
| 214 | 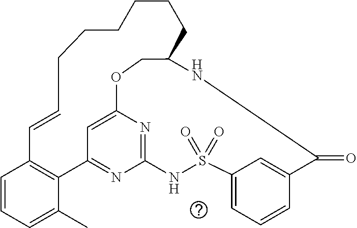 [SMILES: Cc1cccc2c1-c1cc3nc(n1)NS(=O)(=O)c1cccc(c1)C(=O)N[C@H](CCCCCC/C=C/2)CO3] | | | | | | |
| 213 | 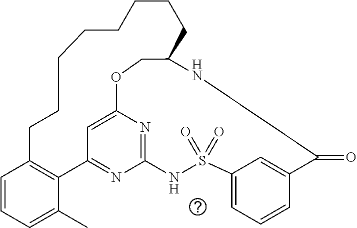 [SMILES: Cc1cccc2c1-c1cc3nc(n1)NS(=O)(=O)c1cccc(c1)C(=O)N[C@H](CCCCCCCC2)CO3] | | | | | +++ | +++ |
| 122 | 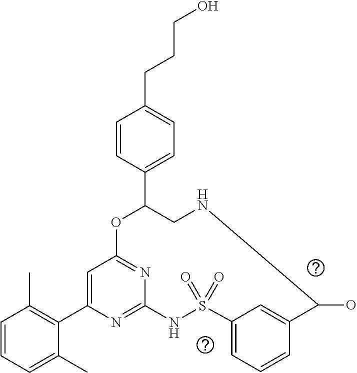 [SMILES: Cc1cccc(C)c1-c1cc2nc(n1)NS(=O)(=O)c1cccc(c1)C(O)NCC(c1ccc(CCCO)cc1)O2] | | | | | ++ | +++ |
| 484 | 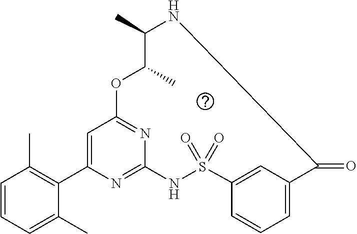 [SMILES: Cc1cccc(C)c1-c1cc2nc(n1)NS(=O)(=O)c1cccc(c1)C(=O)N[C@H](C)[C@H](C)O2] | | | | | + | ++ |
| 483 | 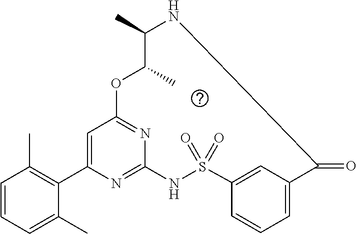 [SMILES: Cc1cccc(C)c1-c1cc2nc(n1)NS(=O)(=O)c1cccc(c1)C(=O)N[C@H](C)[C@H](C)O2] | | | | | ++ | + |
| 67 | 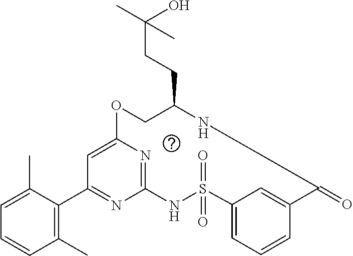 [SMILES: Cc1cccc(C)c1-c1cc2nc(n1)NS(=O)(=O)c1cccc(c1)C(=O)N[C@H](CCC(C)(C)O)CO2] | | | | | + | + |
| 65 | 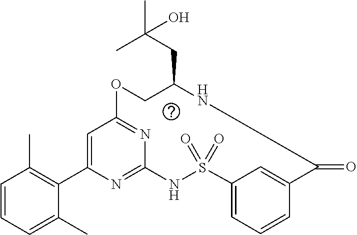 [SMILES: Cc1cccc(C)c1-c1cc2nc(n1)NS(=O)(=O)c1cccc(c1)C(=O)N[C@H](CC(C)(C)O)CO2] | | | | | + | ++ |
| 77 | 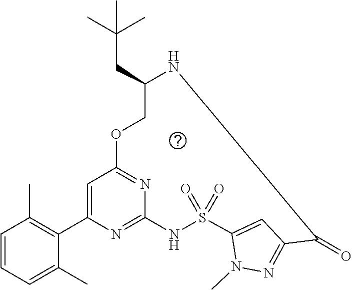 [SMILES: Cc1cccc(C)c1-c1cc2nc(n1)NS(=O)(=O)c1cc(nn1C)C(=O)N[C@H](CC(C)(C)C)CO2] | | | | | ++ | ++ |
| 358 | 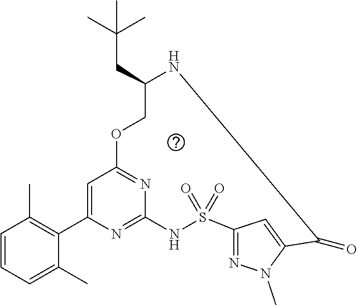 [SMILES: Cc1cccc(C)c1-c1cc2nc(n1)NS(=O)(=O)c1cc(n(C)n1)C(=O)N[C@H](CC(C)(C)C)CO2] | | | | | +++ | +++ |
| 357 | 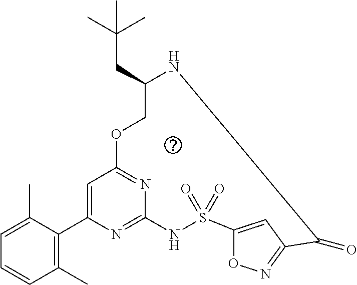 [SMILES: Cc1cccc(C)c1-c1cc2nc(n1)NS(=O)(=O)c1cc(no1)C(=O)N[C@H](CC(C)(C)C)CO2] | | | | | ++ | ++ |
| 295 | 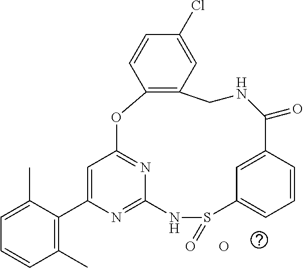 | | | | | +++ | +++ |

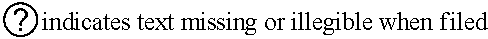

The following tables represents CFTR modulating activity for representative compounds of the disclosure generated using one or more of the assays disclosed herein (EC₅₀: +++ is <1 μM; ++ is 1−<3 μM; + is 3−<30 μM; and ND is “not detected in this assay.” % Activity: +++ is >60%; ++ is 30-60%; + is <30%).

TABLE 10
Biological activity data

| | | | HBE | HBE |
|---|---|---|---|---|
| | | HBE | Max | Activity |
| Compound | | EC₅₀ | Activity | at 10 |
| Number | Structure | (μM) | (%) | μM (%) |
| 294 | 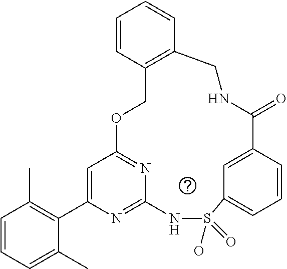 | | | |
| 293 | 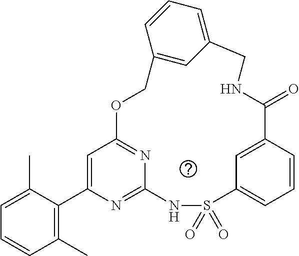 [SMILES: Cc1cccc(C)c1-c1cc2nc(n1)NS(=O)(=O)c1cccc(c1)C(=O)NCc1cccc(c1)CO2] | | | |
| 52 | 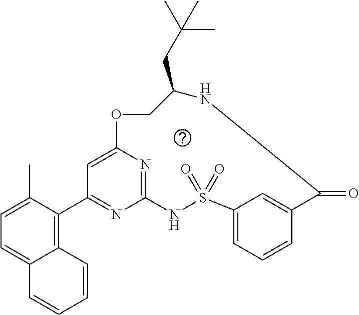 [SMILES: Cc1ccc2ccccc2c1-c1cc2nc(n1)NS(=O)(=O)c1cccc(c1)C(=O)N[C@H](CC(C)(C)C)CO2] | +++ | +++ | |
| 292 | 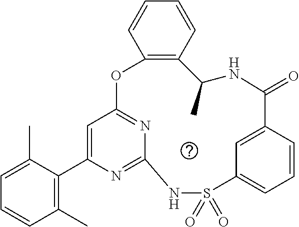 [SMILES: Cc1cccc(C)c1-c1cc2nc(n1)NS(=O)(=O)c1cccc(c1)C(=O)N[C@@H](C)c1ccccc1O2] | | | |
| 291 | 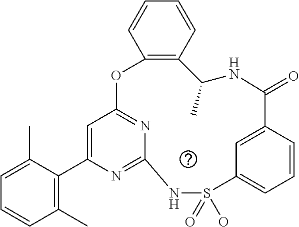 | | | |
| 299 | 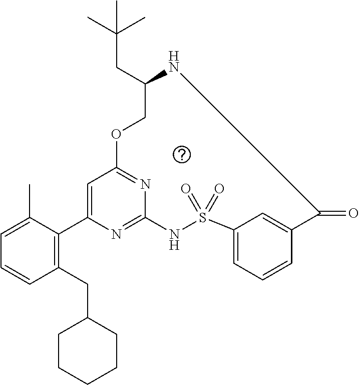 [SMILES: Cc1cccc(CC2CCCCC2)c1-c1cc2nc(n1)NS(=O)(=O)c1cccc(c1)C(=O)N[C@H](CC(C)(C)C)CO2] | +++ | +++ | |
| 49 | 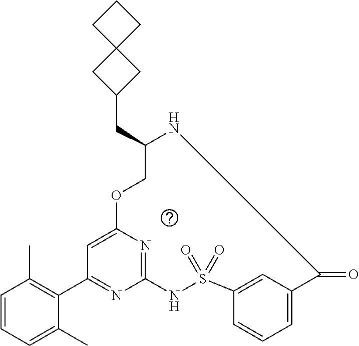 [SMILES: Cc1cccc(C)c1-c1cc2nc(n1)NS(=O)(=O)c1cccc(c1)C(=O)N[C@H](CC1CC3(CCC3)C1)CO2] | | | +++ |
| 208 | 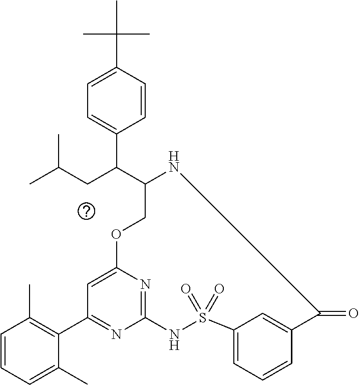 [SMILES: Cc1cccc(C)c1-c1cc2nc(n1)NS(=O)(=O)c1cccc(c1)C(=O)NC(C(CC(C)C)c1ccc(C(C)(C)C)cc1)CO2] | | | +++ |
| 207 | 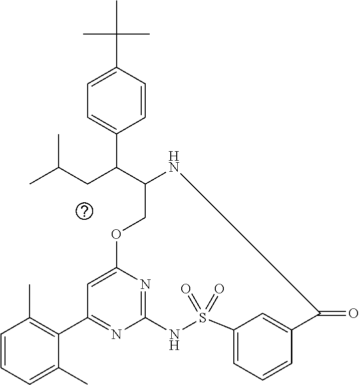 [SMILES: Cc1cccc(C)c1-c1cc2nc(n1)NS(=O)(=O)c1cccc(c1)C(=O)NC(C(CC(C)C)c1ccc(C(C)(C)C)cc1)CO2] | | | + |
| 50 | 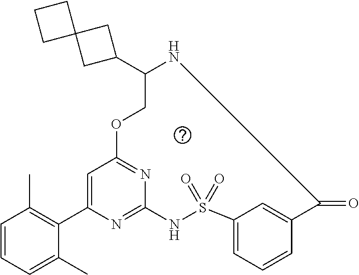 [SMILES: Cc1cccc(C)c1-c1cc2nc(n1)NS(=O)(=O)c1cccc(c1)C(=O)NC(C1CC3(CCC3)C1)CO2] | +++ | +++ | |
| 212 | 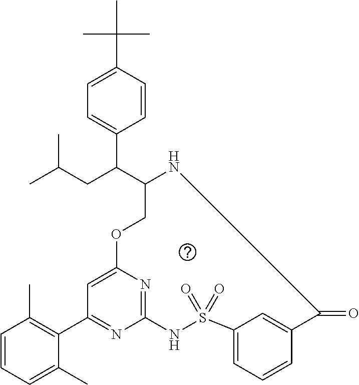 [SMILES: Cc1cccc(C)c1-c1cc2nc(n1)NS(=O)(=O)c1cccc(c1)C(=O)NC(C(CC(C)C)c1ccc(C(C)(C)C)cc1)CO2] | | | |
| 211 | 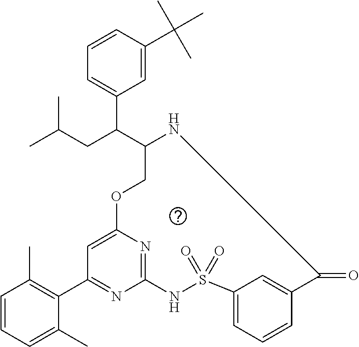 [SMILES: Cc1cccc(C)c1-c1cc2nc(n1)NS(=O)(=O)c1cccc(c1)C(=O)NC(C(CC(C)C)c1cccc(C(C)(C)C)c1)CO2] | +++ | +++ | |
| 210 | 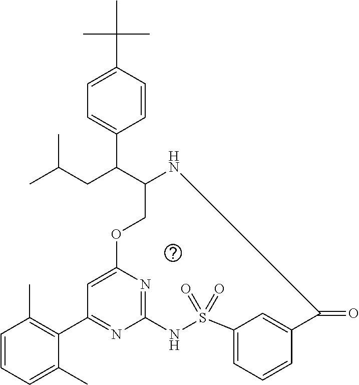 [SMILES: Cc1cccc(C)c1-c1cc2nc(n1)NS(=O)(=O)c1cccc(c1)C(=O)NC(C(CC(C)C)c1ccc(C(C)(C)C)cc1)CO2] | +++ | ++ | |
| 209 | 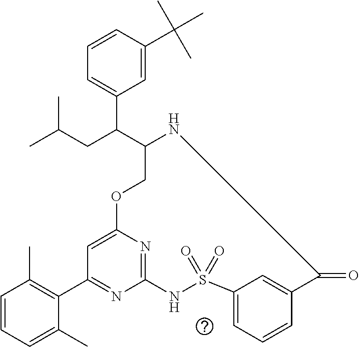 [SMILES: Cc1cccc(C)c1-c1cc2nc(n1)NS(=O)(=O)c1cccc(c1)C(=O)NC(C(CC(C)C)c1cccc(C(C)(C)C)c1)CO2] | +++ | +++ | |
| 298 | 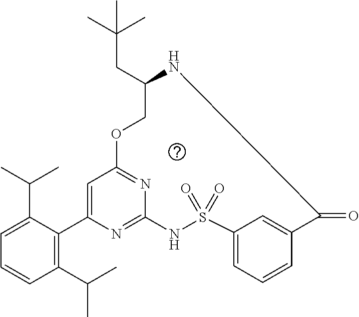 [SMILES: CC(C)c1cccc(C(C)C)c1-c1cc2nc(n1)NS(=O)(=O)c1cccc(c1)C(=O)N[C@H](CC(C)(C)C)CO2] | | | |
| 51 | 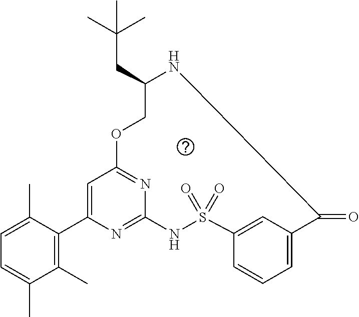 [SMILES: Cc1ccc(C)c(-c2cc3nc(n2)NS(=O)(=O)c2cccc(c2)C(=O)N[C@H](CC(C)(C)C)CO3)c1C] | +++ | +++ | |
| 121 | 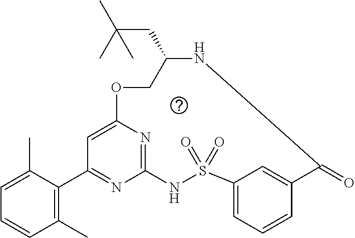 [SMILES: Cc1cccc(C)c1-c1cc2nc(n1)NS(=O)(=O)c1cccc(c1)C(=O)N[C@@H](CC(C)(C)C)CO2] | | | + |

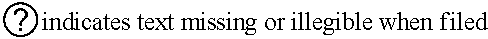

### VI. Synthesis of (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol

### B. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Unless stated to the contrary in the following examples, final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range.

Solid-state NMR (SSNMR) spectra were recorded on a Bruker-Biospin 400 MHz wide-bore spectrometer equipped with Bruker-Biospin 4 mm HFX probe. Samples were packed into 4 mm ZrO₂ rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C cross-polarization (CP) MAS experiment. The fluorine relaxation time was measured using ¹⁹F MAS Ti saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### C. Procedures for the Synthesis of Intermediates

### Intermediate 1: Preparation of methyl 3-[bis(tert-butoxycarbonyl)amino]-6-bromo-5-(trifluoromethyl)pyridine-2-carboxylate

### Step 1: Methyl 3-(benzhydrylideneamino)-5-(trifluoromethyl)pyridine-2-carboxylate

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 hours. The reaction was cooled to room temperature and filtered on a pad of Celite. The cake was washed with EtOAc and solvents were evaporated under reduced pressure to give methyl 3-(benzhydrylideneamino)-5-(trifluoromethyl)pyridine-2-carboxylate (90 g, 84%) as yellow solid. ESI-MS m/z calc. 384.10855, found 385.1 (M+1)⁺; Retention time: 2.24 minutes. LCMS Method: Kinetex C₁₈ 4.6×50 mm 2.6 μM, 2.0 mL/min, 95% H₂O (0.1% formic acid)+5% acetonitrile (0.1% formic acid) to 95% acetonitrile (0.1% formic acid) gradient (2.0 min) then held at 95% acetonitrile (0.1% formic acid) for 1.0 minute.

### Step 2: Methyl 3-amino-5-(trifluoromethyl)pyridine-2-carboxylate

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hours, then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL), and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H) ppm. ¹⁹F NMR (282 MHz, CDCl₃) δ-63.23 (s, 3F) ppm. ESI-MS m/z calc. 220.046, found 221.1 (M+1)⁺; Retention time: 1.62 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which was back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s., 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈4.6×50 mm 2.6 μM. Temp: 45° C., Flow: 2.0 mL/min, Run Time: 6 minutes. Mobile Phase: Initial 95% H₂O (0.1% formic acid) and 5% acetonitrile (0.1% formic acid) linear gradient to 95% acetonitrile (0.1% formic acid) for 4.0 minutes, then held at 95% acetonitrile (0.1% formic acid) for 2.0 minutes.

### Step 4: Methyl 3-[bis(tert-butoxycarbonyl)amino]-6-bromo-5-(trifluoro methyl)pyridine-2-carboxylate

A mixture of methyl 3-amino-6-bromo-5-(trifluoromethyl)pyridine-2-carboxylate (5 g, 15.549 mmol), (Boc)₂O (11 g, 11.579 mL, 50.402 mmol), DMAP (310 mg, 2.5375 mmol) and CH₂Cl₂ (150 mL) was stirred at room temperature overnight. The reaction mixture was concentrated under reduced pressure, and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ-63.9 (s, 3F) ppm. ESI-MS m/z calc. 498.06134, Retention time: 2.34 minutes. LCMS Method: Kinetex C₁₈4.6×50 mm 2.6 μM. Temp: 45° C., Flow: 2.0 mL/min, Run Time: 3 minutes. Mobile Phase: Initial 95% H₂O (0.1% formic acid) and 5% acetonitrile (0.1% formic acid) linear gradient to 95% acetonitrile (0.1% formic acid) for 2.0 minutes, then held at 95% acetonitrile (0.1% formic acid) for 1.0 minute.

### Intermediate 2: Preparation of 6-bromo-3-(tert-butoxycarbonylamino)-5-(trifluoromethyl)pyridine-2-carboxylic Acid

### Step 1: 6-Bromo-3-(tert-butoxycarbonylamino)-5-(trifluoromethyl)pyridine-2-carboxylic acid

To a mixture of methyl 3-[bis(tert-butoxycarbonyl)amino]-6-bromo-5-(trifluoromethyl)pyridine-2-carboxylate (247 g, 494.7 mmol) in THE (1.0 L) was added a solution of LiOH (47.2 g, 1.971 mol) in water (500 mL). The mixture was stirred at ambient temperature for 18 hours, affording a yellow slurry. The mixture was cooled with an ice-bath and slowly acidified with HCl (1000 mL of 2 M, 2.000 mol) keeping the reaction temperature <15° C. The mixture was diluted with heptane (1.5 L), mixed and the organic phase separated. The aqueous phase was extracted with heptane (500 mL). The combined organic phases were washed with brine, dried over MgSO₄, filtered and concentrated in vacuo. The crude oil was dissolved in heptane (600 mL), seeded and stirred at ambient temperature for 18 hours, affording a thick slurry. The slurry was diluted with cold heptane (500 mL) and the precipitate collected using a medium frit. The filter cake was washed with cold heptane and air dried for 1 hour, then in vacuo at 45° C. for 48 hours to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 384.9 (M+1)⁺; Retention time: 2.55 minutes. LCMS Method Detail: Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 3: Preparation of 2-Benzyloxy-2-(trifluoromethyl)hex-5-enoic Acid

### Step 1: Ethyl 2-hydroxy-2-(trifluoromethyl)hex-5-enoate

To a solution of ethyl 3,3,3-trifluoro-2-oxo-propanoate (25.15 g, 147.87 mmol) in Et₂O (270 mL) at −78° C. was added bromo(but-3-enyl)magnesium in THE (190 mL of 0.817 M, 155.23 mmol) dropwise over a period of 1.5 hours (inner temperature −72° C. to −76° C.). The mixture was stirred at −78° C. for 20 minutes. The dry ice-acetone bath was removed. The mixture was slowly warm to 5° C. over 1 hour, added to a mixture of 1 N aqueous HCl (170 mL) and crushed ice (150 g) (pH=4). The two layers were separated. The organic layer was concentrated, and the residue was combined with aqueous phase and extracted with EtOAc (2×150 mL). The combined organic phase was washed with 5% aqueous NaHCO₃(50 mL) and brine (20 mL), and dried with Na₂SO₄. The mixture was filtered and concentrated, and co-evaporated with THE (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (ddt, J=17.0, 10.4, 6.4 Hz, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ-78.74 (s, 3F) ppm.

### Step 2: Ethyl 2-benzyloxy-2-(trifluoromethyl)hex-5-enoate

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 minutes. Benzyl bromide (24.13 g, 141.08 mmol) and TBAI (8.68 g, 23.500 mmol) were added. The mixture was stirred at room temperature overnight. NH₄Cl (3 g, 0.6 eq) was added. The mixture was stirred for 10 minutes. 30 mL of EtOAc was added, then ice-water was added (400 g). The mixture was extracted with CH₂Cl₂ and the combined organic layers were concentrated. Purification by silica gel chromatography (0-20% CH₂Cl₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. 19F NMR (282 MHz, CDCl₃) δ-70.5 (s, 3F) ppm. ESI-MS m/z calc. 316.12863, found 317.1 (M+1)⁺; Retention time: 2.47 minutes. LCMS Method: Kinetex C₁₈ 4.6×50 mm 2.6 μM. Temp: 45° C., Flow: 2.0 mL/min, Run Time: 3 minutes. Mobile Phase: Initial 95% H₂O (0.1% formic acid) and 5% acetonitrile (0.1% formic acid) linear gradient to 95% acetonitrile (0.1% formic acid) for 2.0 minutes, then held at 95% acetonitrile (0.1% formic acid) for 1.0 minute.

### Step 3: 2-Benzyloxy-2-(trifluoromethyl)hex-5-enoic Acid

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as pale yellow oil. ¹H NMR (300 MHz, DMSO-d₆) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d₆) δ-70.29 (s, 3F) ppm. ESI-MS m/z calc. 288.09732, found 287.1 (M−1); Retention time: 3.1 minutes. LCMS Method: Kinetex Polar C₁₈ 3.0×50 mm 2.6 μm, 6 min, 5-95% acetonitrile in H₂O (0.1% formic acid) 1.2 mL/min.

### Intermediate 4: Preparation of (2R)-2-benzyloxy-2-(trifluoromethyl)hex-5-enoic Acid

### Step-1: (2R)-2-Benzyloxy-2-(trifluoromethyl)hex-5-enoic acid; (R)-4-quinolyl-[(2S,4S)-5-vinylquinuclidin-2-yl]methanol

To a N₂ purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 hours, then stirred overnight at 20° C. The material was then filtered and the cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N₂ purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (45 L, 20 vol based on salt solid wt) was slurried and added to a reactor, stirred under N₂ purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid; (R)-4-quinolyl-[(2S,4S)-5-vinylquinuclidin-2-yl]methanol (1.886 kg, >98.0% ee) as an off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.1% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic Acid

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring for 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ-71.63 (br s, 3F) ppm. ESI-MS m/z calc. 288.0973, found 287.0 (M−1); Retention time: 2.15 minutes. LCMS Method: Kinetex Polar C₁₈ 3.0×50 mm 2.6 μm, 3 min, 5-95% acetonitrile in H₂O (0.1% formic acid) 1.2 mL/min.

### Intermediate 5: Preparation of (2R)-2-benzyloxy-2-(trifluoromethyl)hex-5-enehydrazide

### Step 1: tert-Butyl N-[[(2R)-2-benzyloxy-2-(trifluoromethyl)hex-5-enoyl]amino]carbamate

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 minutes. To the mixture was added tert-butyl N-aminocarbamate (200 g, 1.513 mol) (slight exotherm upon addition) and the mixture was stirred at ambient temperature for 16 hours. The reaction was poured into ice water (5 L). The resultant precipitate was collected by filtration and washed with water. The solid was dissolved in EtOAc (2 L) and washed with brine. The organic phase was dried over MgSO₄, filtered, and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% EtOAc/hexanes) provided tert-butyl N-[[(2R)-2-benzyloxy-2-(trifluoromethyl)hex-5-enoyl]amino] carbamate (464 g, 91%, combined with product from crystallization). ESI-MS m z calc. 402.17664, found 303.0 (M+1-Boc)⁺; Retention time: 2.68 minutes. Final purity was determined by reversed phase UPLC using an Acquity UPLC BEH C₁₈ column (50×2.1 mm, 1.7 m particle) made by Waters (pn: 186002350) and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: (2R)-2-Benzyloxy-2-(trifluoromethyl)hex-5-enehydrazide

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 hours. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with 1 L of brine and the combined aqueous phases were extracted with isopropyl acetate (1 L). The combined organic phases were dried over MgSO₄, filtered and concentrated in vacuo affording a dark yellow oil of (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 6: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T3P (622 g of 50% w/w, 977.4 mmol) using an ice-water bath to keep the temperature <35° C. (temperature rose to 34° C.) and the reaction mixture was stirred at ambient temperature for 18 hours. Additional DIEA (100 mL, 574.1 mmol) and T3P (95 g, 298.6 mmol) were added and stirred at ambient temperature for 2 days. Starting material was still observed and additional T3P (252 g, 792 mmol) was added and stirred for 5 days. The reaction was quenched with the slow addition of water (2.5 L) and the mixture stirred for 30 minutes. The organic phase was separated, and the aqueous phase extracted with EtOAc (2 L). The combined organic phases were washed with brine, dried over MgSO₄, filtered and concentrated in vacuo. The crude product was dissolved in MTBE (300 mL) and diluted with heptane (3 L), the mixture stirred at ambient temperature for 12 hours affording a light yellow slurry. The slurry was filtered, and the resultant solid was air dried for 2 hours, then in vacuo at 40° C. for 48 hours. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 hour. The mixture was stirred at 70° C. for 9 hours then additional p-toluenesulfonyl chloride (6.5 g, 34.09 mmol) was added. The mixture was stirred for a total of 24 hours then allowed to cool to ambient temperature. Acetonitrile was removed in vacuo affording a dark orange oil which was diluted with EtOAc (1.5 L) and water (1.5 L). The organic phase was separated and washed with 500 mL of 1M HCl, 500 mL of brine, dried over MgSO₄, filtered and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m/z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed DMAP (2.09 g, 17.11 mmol). A solution of di-tert-butyl dicarbonate (111.6 g, 511.3 mmol) in MTBE (250 mL) was added over approximately 8 minutes, and the resulting mixture was stirred for additional 30 minutes. 1 L of water was added and the layers separated. The organic layer was washed with KHSO₄ (886 mL of 0.5 M, 443.0 mmol), 300 mL brine, dried with MgSO₄ and most (>95%) of the MTBE was evaporated by rotary evaporation at 45° C., leaving a thick oil. 1.125 L of heptane was added, spun in the 45° C. rotovap bath until dissolved, then evaporated out 325 mL of solvent by rotary evaporation. The rotovap bath temp was allowed to drop to room temperature and product started crystallizing out during the evaporation. Then the flask was placed in a −20° C. freezer overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m/z calc. 750.14874, found 751.1 (M+1)⁺; Retention time: 3.76 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 8: Preparation of tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 hours. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated and the organic phase washed with water (3×3 L) and with brine (1×2.5 L). The organic phase was dried with MgSO₄, filtered, and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotovap at 100 mbar pressure at room temperature for 1.5 hours. The solid mass was stirred mechanically for 2 hours at room temperature, resultant thick fine suspension was filtered, washed with dry ice cold heptane and dried under vacuum at 45° C. with a nitrogen bleed for 16 hours to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d₆) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m/z calc. 688.23315, found 689.0 (M+1)⁺; Retention time: 3.32 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### D. Preparation of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 minutes. For the next approximately 2 hours, the mixture was cooled to room temperature. During this cooling period, after the first 10 minutes, triphenylphosphine (6.07 g, 23.14 mmol) was added. After a further 1 hour, additional triphenylphosphine (3.04 g, 11.59 mmol) was added. After a further 23 minutes, DIAD (2.24 mL, 11.57 mmol) was added. After the ˜2 hour cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 minutes, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m/z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture)

The following reaction was run, split equally between two, 12 L reaction flasks run in parallel. Mechanical stirring was employed, and reactions were subjected to a constant nitrogen gas purge using a coarse porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st} Generation Catalyst (9 g, 10.94 mmol in each flask) was added to each reaction and stirred at 400 rpm while setting an internal temperature control to 75° C. with strong nitrogen purging (both reactions reached −75° C. after approximately 20 min). After 5 hours, 15 minutes, the internal temperature control was set to 45° C. After approximately 2 hours, 2-sulfanylpyridine-3-carboxylic acid (11 g, 70.89 mmol in each flask) was added to each flask, followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 hours, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg Cis column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m/z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 hours. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL), charged with fresh 10% Pd/C (50% water wet, 2.2 g of 5% w/w, 1.034 mmol) and stirred vigorously under hydrogen (balloon) for 6 hours, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m/z calc. 730.2801, found 731.0 (M+1)⁺; Retention time: 3.89 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5% w/w, 0.8457 mmol) then cycled 3 times between vacuum/nitrogen and 3 times between vacuum/hydrogen and then stirred vigorously under hydrogen (balloon) at room temperature for 18 hours. The mixture was cycled 3 times between vacuum/nitrogen, filtered over Celite, washing with ethanol, and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5] nonadeca-1(18),2,4,14,16-pentaen-17-yl]carbamate an off-white solid. 1H NMR and MS confirmed the expected product. CFTR modulatory activity was confirmed using a standard Ussing Chamber Assay for CFTR potentiator activity.

### Other Embodiments

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound of Formula I: 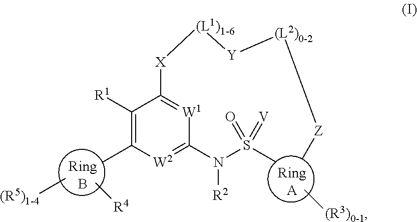
a tautomer thereof, a deuterated derivative of the compound or tautomer, or a pharmaceutically acceptable salt of any of the foregoing, wherein:
Ring A is selected from:
C₆-C₁₀ aryl,
C₃-C₁₀ cycloalkyl,
3- to 10-membered heterocyclyl, and
5- to 10-membered heteroaryl;
Ring B is selected from:
C₆-C₁₀ aryl,
C₃-C₁₀ cycloalkyl,
3- to 10-membered heterocyclyl, and
5- to 10-membered heteroaryl;
V is selected from O and NH;
W¹ is selected from N and CH;
W² is selected from N and CH; provided that at least one of W¹ and W² is N;
Z is selected from O, NR^{ZN}, and C(R^{ZC})₂, provided that when L² is absent, Z is C(R^{ZC})₂;
each L¹ is independently selected from C(R^{L1})₂ and 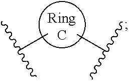
each L² is independently selected from C(R^{L2})₂;
Ring C is selected from C₆-C₁₀ aryl optionally substituted with 1-3 groups independently selected from:
halogen,
C₁-C₆ alkyl, and
N(R^{N})₂;
each R³ is independently selected from:
halogen,
C₁-C₆ alkyl,
C₁-C₆ alkoxy,
C₃-C₁₀ cycloalkyl,
C₆-C₁₀ aryl optionally substituted with 1-3 groups independently selected from C₁-C₆ alkyl, and
3- to 10-membered heterocyclyl;
R⁴ is selected from hydrogen and C₁-C₆ alkyl;
each R⁵ is independently selected from:
hydrogen,
halogen,
hydroxyl,
N(R^{N})₂,
—SO—Me,
—CH═C(R^{Lc})₂, wherein both R^{LC} are taken together to form a C₃-C₁₀ cycloalkyl,
C₁-C₆ alkyl optionally substituted with 1-3 groups independently selected from:
hydroxyl,
C₁-C₆ alkoxy optionally substituted with 1-3 groups independently selected from C₁-C₆ alkoxy and C₆-C₁₀ aryl,
C₃-C₁₀ cycloalkyl,
—(O)₀₋₁—(C₆-C₁₀ aryl) optionally substituted with 1-3 groups independently selected from C₁-C₆ alkyl and C₁-C₆ alkoxy,
3- to 10-membered heterocyclyl, and
N(R^{N})₂,
C₁-C₆ alkoxy optionally substituted with 1-3 groups independently selected from:
halogen,
C₆-C₁₀ aryl, and
C₃-C₁₀ cycloalkyl optionally substituted with 1-3 groups independently selected from C₁-C₆ fluoroalkyl,
C₁-C₆ fluoroalkyl,
C₃-C₁₀ cycloalkyl,
C₆-C₁₀ aryl, and
3- to 10-membered heterocyclyl;
R^{ZN} is selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-C₁₀ aryl, and N(R^{N})₂,
C₁-C₆ fluoroalkyl,
C₁-C₆ alkoxy, and
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF₅,
N(R^{N})₂,
P(O)Me₂,
—(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
—(O)₀₋₁—(C₆-C₁₀ aryl), and
—(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl (optionally substituted with 1-3 groups independently selected from oxo and C₁-C₆ alkoxy),
C₁-C₆ alkoxy,
C₁-C₆ fluoroalkyl,
C₆-C₁₀ aryl optionally substituted with 1-3 groups independently selected from halogen, and
5- to 10-membered heteroaryl, and
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3-SiMe₃), and N(R^{N})₂,
C₁-C₆ alkoxy optionally substituted with 1-3 groups independently selected from hydroxyl, oxo, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
—(O)₀₋₁—(C₆-C₁₀ aryl),
—(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
5- to 10-membered heteroaryl optionally substituted with 1-4 groups independently selected from C₁-C₆ alkyl and C₃-C₁₀ cycloalkyl,
C₁-C₆ fluoroalkyl,
C₃-C₁₀ cycloalkyl optionally substituted with 1-3 groups independently selected from:
hydroxyl,
oxo,
halogen,
cyano,
N(R^{N})₂,
C₁-C₆ alkyl optionally substituted with 1-3 groups independently selected from:
hydroxyl,
oxo,
N(R^{N})₂,
C₁-C₆ alkoxy, and
C₆-C₁₀ aryl,
C₁-C₆ alkoxy optionally substituted with 1-3 groups independently selected from halogen, oxo, C₆-C₁₀ aryl, and N(R^{N})₂,
halogen,
C₃-C₁₀ cycloalkyl,
3- to 10-memember heterocyclyl optionally substituted with 1-3 groups independently selected from C₁-C₆ alkyl, and
5- to 10-membered heteroaryl optionally substituted with 1-3 groups independently selected from:
hydroxyl,
cyano,
oxo,
halogen,
N(R^{N})₂,
C₁-C₆ alkyl optionally substituted with 1-3 groups independently selected from hydroxyl, oxo, C₁-C₆ alkoxy, and N(R^{N})₂,
C₁-C₆ alkoxy optionally substituted with 1-3 groups independently selected from hydroxyl, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
C₆-C₁₀ aryl, and
3- to 10-membered heterocyclyl optionally substituted with 1-3 groups independently selected from C₁-C₆ alkyl,
C₆-C₁₀ aryl,
3- to 10-membered heterocyclyl optionally substituted with 1-3 groups independently selected from:
oxo,
C₁-C₆ alkyl optionally substituted with 1-3 groups independently selected from:
oxo,
hydroxyl,
N(R^{N})₂,
C₁-C₆ alkoxy optionally substituted with 1-3 groups independently selected from halogen and C₆-C₁₀ aryl, and
—(O)₀₋₁—(C₃-C₁₀ cycloalkyl),
C₁-C₆ fluoroalkyl,
C₃-C₁₀ cycloalkyl optionally substituted with 1-3 groups independently selected from halogen, and
3- to 10-membered heterocyclyl,
5- to 10-membered heteroaryl optionally substituted with 1-3 groups independently selected from:
halogen,
C₁-C₆ alkyl optionally substituted with 1-3 groups independently selected from oxo, C₁-C₆ alkoxy, and N(R^{N})₂, and
3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
R^{F};
each R^{ZC} is independently selected from:
hydrogen,
C₁-C₆ alkyl optionally substituted with 1-3 groups independently selected from C₆-C₁₀ aryl (optionally substituted with 1-3 groups independently selected from C₁-C₆ alkyl),
C₆-C₁₀ aryl optionally substituted with 1-3 groups independently selected from C₁-C₆ alkyl, and
R^{F};
or two R^{ZC} are taken together to form an oxo group;
each R^{L1} is independently selected from:
hydrogen,
N(R^{N})₂, provided that two N(R^{N})₂ are not bonded to the same carbon,
C₁-C₉ alkyl optionally substituted with 1-3 groups independently selected from:
halogen,
hydroxyl,
oxo,
N(R^{N})₂,
C₁-C₆ alkoxy optionally substituted with 1-3 groups independently selected from C₆-C₁₀ aryl,
C₃-C₁₀ cycloalkyl optionally substituted with 1-3 groups independently selected from halogen and C₁-C₆ fluoroalkyl,
C₆-C₁₀ aryl optionally substituted with 1-3 groups independently selected from C₁-C₆ alkyl, and
3- to 10-membered heterocyclyl optionally substituted with 1-3 groups independently selected from C₁-C₆ alkyl (optionally substituted with 1-3 groups independently selected from hydroxyl and oxo),
C₃-C₁₀ cycloalkyl,
C₆-C₁₀ aryl optionally substituted with 1-4 groups independently selected from:
halogen,
cyano,
SiMe₃,
POMe₂,
C₁-C₇ alkyl optionally substituted with 1-3 groups independently selected from:
hydroxyl,
oxo,
cyano,
SiMe₃,
N(R^{N})₂, and
C₃-C₁₀ cycloalkyl optionally substituted with 1-3 groups independently selected from C₁-C₆ fluoroalkyl,
C₁-C₆ alkoxy optionally substituted with 1-3 groups independently selected from:
C₃-C₁₀ cycloalkyl optionally substituted with 1-3 groups independently selected from C₁-C₆ fluoroalkyl, and
C₁-C₆ alkoxy,
C₁-C₆ fluoroalkyl,
C₃-C₁₀ cycloalkyl optionally substituted with 1-3 groups independently selected from C₁-C₆ alkyl and C₁-C₆ fluoroalkyl,
C₆-C₁₀ aryl,
3- to 10-membered heterocyclyl optionally substituted with 1-3 groups independently selected from C₁-C₆ alkyl, and
5- to 10-membered heteroaryl,
3- to 10-membered heterocyclyl optionally substituted with 1-3 groups independently selected from:
C₁-C₆ alkyl optionally substituted with 1-3 groups independently selected from:
oxo, and
C₁-C₆ alkoxy,
5- to 10-membered heteroaryl optionally substituted with 1-3 groups independently selected from:
C₁-C₆ alkyl optionally substituted with 1-3 groups independently selected from:
C₃-C₁₀ cycloalkyl optionally substituted with 1-3 groups independently selected from C₁-C₆ fluoroalkyl, and
C₆-C₁₀ aryl optionally substituted with 1-3 groups independently selected from C₁-C₆ alkyl, and
R^{F};
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
hydrogen,
C₁-C₈ alkyl optionally substituted with 1-3 groups independently selected from:
oxo,
halogen,
hydroxyl,
NH₂,
NHMe,
NMe₂,
NHCOMe,
C₁-C₆ alkoxy optionally substituted with 1-3 groups independently selected from C₆-C₁₀ aryl,
—(O)₀₋₁—(C₃-C₁₀ cycloalkyl),
C₆-C₁₀ aryl optionally substituted with 1-3 groups independently selected from halogen and C₁-C₆ alkyl, and
3- to 14-membered heterocyclyl optionally substituted with 1-4 groups independently selected from oxo and C₁-C₆ alkyl,
5- to 14-membered heteroaryl optionally substituted with 1-4 groups independently selected from oxo and C₁-C₆ alkyl,
C₃-C₁₀ cycloalkyl optionally substituted with 1-3 groups independently selected from:
hydroxyl,
NH₂, and
NHMe,
C₁-C₆ alkyl optionally substituted with 1-3 groups independently selected from hydroxyl, and
C₆-C₁₀ aryl, and
3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
hydroxyl,
oxo,
cyano,
C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
C₁-C₆ alkoxy, and
C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
C₃-C₁₀ cycloalkyl optionally substituted with 1-3 groups independently selected from C₁-C₆ alkyl,
C₆-C₁₀ aryl optionally substituted with 1-3 groups independently selected from:
halogen,
C₁-C₆ alkyl,
N(R^{N})₂, and
3- to 10-membered heterocyclyl optionally substituted with 1-3 groups independently selected from hydroxyl,
3- to 11-membered heterocyclyl optionally substituted with 1-3 groups independently selected from:
oxo,
N(R^{N})₂,
C₁-C₉ alkyl optionally substituted with 1-4 groups independently selected from:
oxo,
halogen,
hydroxyl,
N(R^{N})₂,
SO₂—(C₁-C₆ alkyl),
C₁-C₆ alkoxy optionally substituted with 1-3 groups independently selected from halogen, C₆-C₁₀ aryl,
C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
—(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
C₃-C₁₂ cycloalkyl optionally substituted with 1-4 groups independently selected from halogen, C₁-C₆ alkyl, and C₁-C₆ fluoroalkyl,
C₆-C₁₀ aryl,
3- to 10-membered heterocyclyl, and
5- to 10-membered heteroaryl optionally substituted with 1-3 groups independently selected from C₁-C₆ alkoxy and C₁-C₆ fluoroalkyl, and
5- to 12-membered heteroaryl optionally substituted with 1-3 groups independently selected from C₁-C₆ alkyl and C₁-C₆ fluoroalkyl.

2. A compound of Formula Ia: 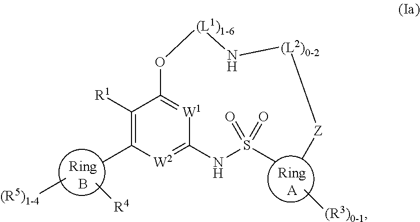
a tautomer thereof, a deuterate derivative of the compound or tautomer, or a pharmaceutically acceptable salt of any of the foregoing, wherein Ring A, Ring B, W¹, W², Z, L¹, L², R³, R⁴, and R⁵ are defined as according to claim 1.

3. A compound of Formula IIa: 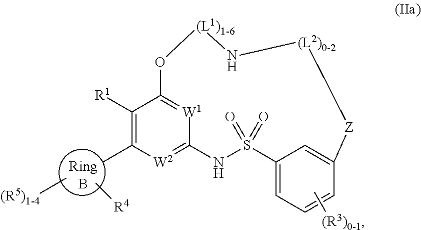
a tautomer thereof, a deuterated derivative of the compound or tautomer, or a pharmaceutically acceptable salt of any of the foregoing, wherein Ring B, W¹, W², Z, L¹, L², R³, R⁴, and R⁵ are defined as according to claim 1.

4. A compound of Formula IIb: 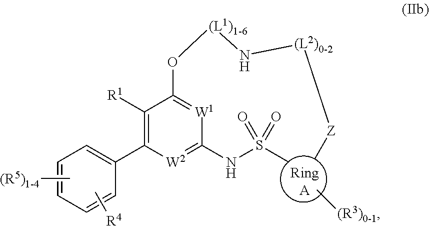
a tautomer thereof, a deuterated derivative of the compound or tautomer, or a pharmaceutically acceptable salt of any of the foregoing, wherein Ring A, W¹, W², Z, L¹, L², R³, R⁴, and R⁵ are defined as according to claim 1.

5. A compound of Formula III can be depicted as: 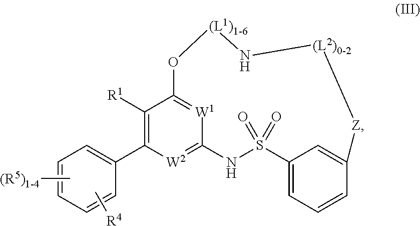
a tautomer thereof, a deuterated derivative of the compound or tautomer, or a pharmaceutically acceptable salt of any of the foregoing, wherein W¹, W², Z, L¹, L², R³, R⁴, and R⁵ are defined as according to claim 1.

6. A compound of Formula IV: 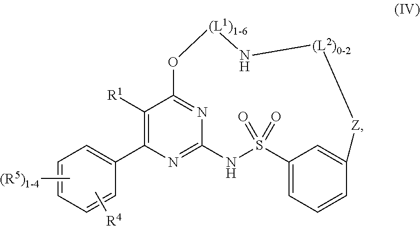
a tautomer thereof, a deuterated derivative of the compound or tautomer, or a pharmaceutically acceptable salt of any of the foregoing, wherein Z, L¹, L², R³, R⁴, and R⁵ are defined as according to claim 1.

7. A compound of Formula V: 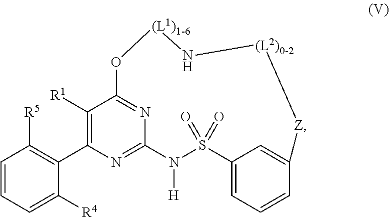
a tautomer thereof, a deuterated derivative of the compound or tautomer, or a pharmaceutically acceptable salt of any of the foregoing, wherein Z, L¹, L², R³, R⁴, and R⁵ are defined as according to claim 1.

8. A compound of Formula VI: 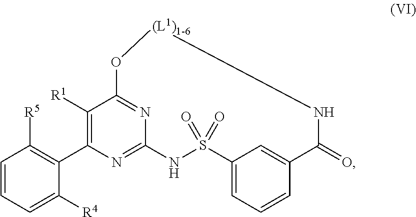
a tautomer thereof, a deuterated derivative of the compound or tautomer, or a pharmaceutically acceptable salt of any of the foregoing, wherein L¹, R³, R⁴, and R⁵ are defined as according to claim 1.

9. The compound, tautomer, deuterated derivative, or pharmaceutically acceptable salt according to any one of claims 1 to 8, selected from compounds of Formulae I, Ia, IIa, IIb, III, IV, V, and VI, and deuterated derivatives thereof and pharmaceutically acceptable salts of any of the foregoing.

10. The compound, tautomer, deuterated derivative, or pharmaceutically acceptable salt according to any one of claims 1 to 9, selected from Compounds 1-496 (Tables 3-5 and 7-10), deuterated derivatives thereof and pharmaceutically acceptable salts of any of the foregoing.

11. A pharmaceutical composition comprising the compound, tautomer, deuterated derivative, or pharmaceutically acceptable salt according to any one of claims 1 to 10, and a pharmaceutically acceptable carrier.

12. The pharmaceutical composition of claim 11, further comprising one or more additional therapeutic agents.

13. The pharmaceutical composition of claim 12, wherein the one or more additional therapeutic agents are selected one or more CFTR modulators.

14. The pharmaceutical composition of claim 13, wherein the one or more CFTR modulators are selected from tezacaftor, ivacaftor, deutivacaftor, lumacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof.

15. A method of treating cystic fibrosis comprising administering to a patient in need thereof the compound, tautomer, deuterated derivative, or pharmaceutically acceptable salt according to any one of claims 1 to 10, or a pharmaceutical composition according to any one of claims 11 to 14.

16. The method of claim 15, further comprising administering to the patient one or more additional therapeutic agents prior to, concurrent with, or subsequent to the compound, tautomer, deuterated derivative, or pharmaceutically acceptable salt according to any one of claims 1 to 10, or the pharmaceutical composition according to any one of claim 11.

17. The method of claim 16, wherein the the one or more additional therapeutic agents are selected from CFTR modulators.

18. The method of claim 17, wherein the one or more additional CFTR modulators is (are) selected from tezacaftor, ivacaftor, deutivacaftor, lumacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

19. The compound, tautomer, deuterated derivative, or pharmaceutically acceptable salt according to any one of claims 1 to 10, or the pharmaceutical composition according to any one of claims 11 to 14 for use in the treatment of cystic fibrosis.

20. The compound, tautomer, deuterated derivative, or pharmaceutically acceptable salt according to any one of claims 1 to 10, or the pharmaceutical composition according to any one of claims 11 to 14 for use in the manufacture of a medicament for the treatment of cystic fibrosis.
